SEQUENCE LISTING (2 sequences)

SEQ ID NO: 1 - protein, 1210 aa, Artificial [sequence omitted]

SEQ ID NO: 2 - protein, 1255 aa, Artificial [sequence omitted]

(19) United States
(12) Patent Application Publication
(10) Pub. No.: US 2024/0101576 A1
(43) Pub. Date: Mar. 28, 2024

(54) HETEROCYCLIC INHIBITORS OF EGFR AND/OR HER2, FOR USE IN THE TREATMENT OF CANCER

(71) Applicant: Scorpion Therapeutics, Inc., Boston, MA (US)

(72) Inventors: Angel Guzman-Perez, Belmont, MA (US); Benjamin C. Milgram, Weston, MA (US); Ryan D. White, Somerville, MA (US); David St. Jean, JR., Natick, MA (US)

(21) Appl. No.: 18/030,211

(22) PCT Filed: Oct. 8, 2021
(86) PCT No.: PCT/US2021/054191

§ 371 Date: Apr. 4, 2023

Related U.S. Application Data
(60) Provisional application No. 63/089,965, filed on Oct. 9, 2020.
(60) Provisional application No. 63/151,468, filed on Feb. 19, 2021.

(51) Int. Cl. C07D 519/00; A61K 45/06; C07D 471/04; C07D 471/20; C07D 491/20; G01N 33/574

(52) U.S. Cl. CPC C07D 519/00; A61K 45/06; C07D 471/04; C07D 471/20; C07D 491/20; G01N 33/57415; G01N 2333/485; G01N 2440/14

(57) ABSTRACT

This disclosure provides chemical entities (e.g., a compound or a pharmaceutically acceptable salt, and/or hydrate, and/or cocrystal, and/or drug combination of the compound) that inhibit epidermal growth factor receptor (EGFR, ERBB 1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

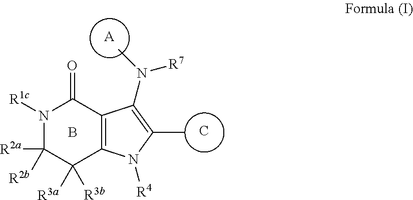

### CROSS REFERENCE TO RELATED APPLICATIONS

This application claims the benefit of U.S. Provisional Application Ser. No. 63/089,965, filed on Oct. 9, 2020; and U.S. Provisional Application Ser. No. 63/151,468, filed on Feb. 19, 2021; each of which is incorporated herein by reference in its entirety.

### TECHNICAL FIELD

This disclosure provides chemical entities (e.g., a compound or a pharmaceutically acceptable salt, and/or hydrate, and/or cocrystal, and/or drug combination of the compound) that inhibit epidermal growth factor receptor (EGFR, ERBB1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

### BACKGROUND

Epidermal growth factor receptor (EGFR, ERBB1) and Human epidermal growth factor receptor 2 (HER2, ERBB2) are members of a family of proteins which regulate cellular processes implicated in tumor growth, including proliferation and differentiation. Several investigators have demonstrated the role of EGFR and HER2 in development and cancer (Reviewed in Salomon, et al., Crit. Rev. Oncol. Hematol. (1995) 19:183-232, Klapper, et al., Adv. Cancer Res. (2000) 77, 25-79 and Hynes and Stern, Biochim. Biophys. Acta (1994) 1198:165-184). EGFR overexpression is present in at least 70% of human cancers, such as non-small cell lung carcinoma (NSCLC), breast cancer, glioma, and prostate cancer. HER2 overexpression occurs in approximately 30% of all breast cancer. It has also been implicated in other human cancers including colon, ovary, bladder, stomach, esophagus, lung, uterus and prostate. HER2 overexpression has also been correlated with poor prognosis in human cancer, including metastasis, and early relapse.

EGFR and HER2 are, therefore, widely recognized as targets for the design and development of therapies that can specifically bind and inhibit tyrosine kinase activity and its signal transduction pathway in cancer cells, and thus can serve as diagnostic or therapeutic agents. For example, EGFR tyrosine kinase inhibitors (TKIs) are effective clinical therapies for EGFR mutant advanced non-small cell lung cancer (NSCLC) patients. However, the vast majority of patients develop disease progression following successful treatment with an EGFR TKI. Common mechanisms of resistance include acquired, secondary mutation T790M, C797S, and EGFR exon 20 insertion mutations. For example, NSCLC tumors can have EGFR exon 20 insertion mutations that are intrinsically resistant to current EGFR TKIs.

Overexpression of another protein, BUB1 (Budding uninhibited by benzimidazole, BUB1) kinase, is often associated with proliferating cells, including cancer cells, and tissues (Bolanos-Garcia V M and Blundell T L, Trends Biochem. Sci. 36, 141, 2010). This protein is an essential part of the complex network of proteins that form the mitotic checkpoint. The major function of an unsatisfied mitotic checkpoint is to keep the anaphase-promoting complex/cyclosome (APC/C) in an inactive state. As soon as the checkpoint gets satisfied the APC/C ubiquitin-ligase targets cyclin B and securin for proteolytic degradation leading to separation of the paired chromosomes and exit from mitosis.

Incomplete mitotic checkpoint function has been linked with aneuploidy and tumorigenesis (see Weaver B A and Cleveland D W, Cancer Res. 67, 10103, 2007; King R W, Biochim Biophys Acta 1786, 4, 2008). In contrast, complete inhibition of the mitotic checkpoint has been recognized to result in severe chromosome missegregation and induction of apoptosis in tumor cells (see Kops G J et al., Nature Rev. Cancer 5, 773, 2005; Schmidt M and Medema R H, Cell Cycle 5, 159, 2006; Schmidt M and Bastians H, Drug Res. Updates 10, 162, 2007). Thus, mitotic checkpoint inhibition through inhibition of BUB1 kinase represents an approach for the treatment of proliferative disorders, including solid tumors such as carcinomas, sarcomas, leukemias and lymphoid malignancies or other disorders, associated with uncontrolled cellular proliferation.

### SUMMARY

This disclosure provides chemical entities (e.g., a compound or a pharmaceutically acceptable salt, and/or hydrate, and/or cocrystal, and/or drug combination of the compound) that inhibit epidermal growth factor receptor (EGFR, ERBB1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). This disclosure also provides compositions containing the same as well as methods of using and making the same.

In one aspect, this disclosure features compounds of Formula (I):

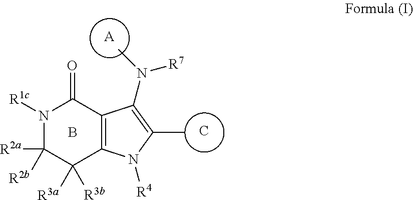

or a pharmaceutically acceptable salt thereof, wherein:

Ring C is selected from the group consisting of:

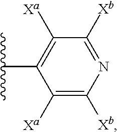

wherein:
each X^{b} is independently X¹, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X¹ and further optionally substituted with from 1-4 R^{c};
2-pyridonyl or 4-pyridonyl, each optionally substituted with X¹ and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 substituents independently selected from the group consisting of oxo and R^{C};
C₃₋₁₀ cycloalkyl or C₃₋₁₀ cycloalkenyl, each of which is optionally substituted with X¹ and further optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{c}; and
C₆₋₁₀ aryl optionally substituted with X¹ and further optionally substituted with from 1-4 R^{c};
X¹ is —(X²)ₘ-L¹-R⁵, wherein: m is 0 or 1;
X² is selected from the group consisting of:
—O—, —N(R^{N})—, or —S(O)₀₋₂;

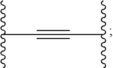 [SMILES: *C#C*]

C₂₋₆ alkenylene optionally substituted with from 1-3 R^{a};
—C(═O)O—*, —C(═O)N(R^{N})—*, or —S(O)₁₋₂N(R^{N})—*.
—OC(═O)—*, —N(R^{N})C(═O)—*, or —N(R^{N})S(O)₁₋₂—*, and
—OC(═O)N(R^{N})—*, —N(R^{N})C(═O)O—*, —N(R^{N})C(═O)N(R^{N})—*, or —N(R^{N})S(O)₁₋₂N(R^{N})—*,
wherein the asterisk represents point of attachment to L¹;
L¹ is selected from the group consisting of: a bond and C₁₋₁₀ alkylene optionally
substituted with from 1-6 R^{a};
R⁵ is selected from the group consisting of:
H;
halo;
—OH,
—NR^{e}R^{f};
—C₁₋₆ alkoxy or —S(O)₀₋₂(C₁₋₆ alkyl), each optionally substituted with from 1-6 R^{a};
—R^{g};
-L⁵-R^{g};
—R^{g2}-R^{W} or —R^{g2}-R^{Y}; and
-L⁵-R^{g2}-R^{W} or -L⁵-R^{g2}-R^{Y};
provided that:
when L¹ is a bond, then R⁵ is selected from the group consisting of: H, —R^{g}, —R^{g2}-R^{W}, and —R^{g2}-R^{Y}; and
X¹ is other than H, —OH, or NH₂,
L⁵ is selected from the group consisting of: —O—, —S(O)₀₋₂, —NH, and —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is selected from the group consisting of:
C₂₋₆ alkenyl; C₂₋₆ alkynyl; or C₃₋₁₀ alkenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
bicyclo[x.y.0]cycloalkyl optionally substituted with from 1-2 R^{c}, wherein x is 1 or 2; and y is an integer from 1 to 6;
R^{Y} is selected from the group consisting of: —R^{g} and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —NR^{e}R^{f}; —R^{g}; -(L^{g})_{g}-R^{g}; and —C₁₋₆ alkoxy or —C₁₋₆ thioalkoxy, each optionally substituted with from 1-6 R^{a}; provided that R^{1c} is other than halo, —CN, or —C(O)OH; or
or two of variables R^{1c}, R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W};
Ring A is R^{g};
R⁴ and R⁷ are independently H or R^{d};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH, —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{f} group;
each occurrence of R′ and R″ is independently selected from the group consisting of: H; —OH; and C₁₋₄ alkyl; and
each occurrence of R^{N} is independently H, C₁₋₃ alkyl, or C₃₋₆ cycloalkyl.

In some embodiments, it is provided that one or more of the following applies:
when R^{2a} and R^{2b} are H or methyl; R^{3a} and R^{3b} are H; Ring C is

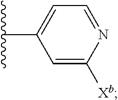

and X^{b} is H, methyl, NH₂, NHC(═O)Me, NHC(═O)iPr, NHC(═O)NHEt,

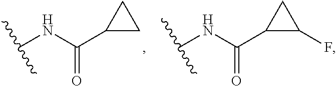

then Ring A is other than unsubstituted phenyl;
when R^{2a}, R^{2b}, R^{3a}, and R^{3b} are each H; Ring C is

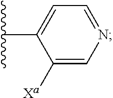

and X^{a} is methyl or F, then Ring A is other than unsubstituted phenyl;
when R^{1c}, R^{2a}, R^{2b}, R^{3a}, and R^{3b} are each H; Ring C is

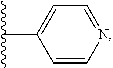 [SMILES: *c1ccncc1]

then Ring A is other than 4-fluorophenyl; and
the compound is other than:

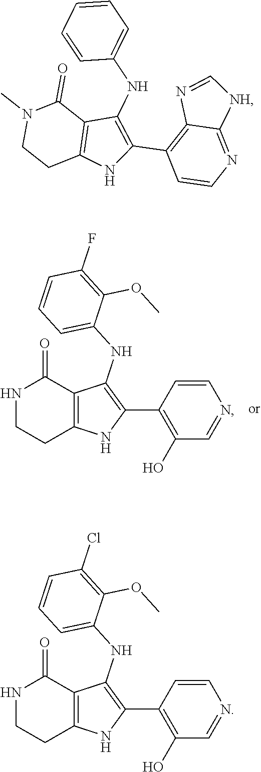 [SMILES: CN1CCc2[nH]c(-c3ccnc4[nH]cnc34)c(Nc3ccccc3)c2C1=O ; COc1c(F)cccc1Nc1c(-c2ccncc2O)[nH]c2c1C(=O)NCC2 ; "or" ; COc1c(Cl)cccc1Nc1c(-c2ccncc2O)[nH]c2c1C(=O)NCC2]

In one aspect, the disclosure features A compound of Formula (I):

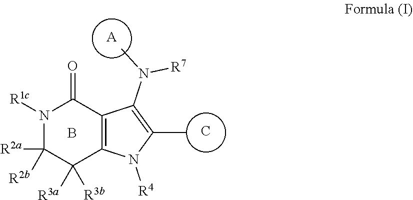

or a pharmaceutically acceptable salt thereof, wherein:

Ring C is selected from the group consisting of:

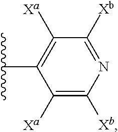

wherein:
each X^{b} is independently X¹, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X¹ and further optionally substituted with from 1-4 R^{c};
2-pyridonyl or 4-pyridonyl, each optionally substituted with X¹ and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 substituents independently selected from the group consisting of oxo and R^{c};
C₃₋₁₀ cycloalkyl or C₃₋₁₀ cycloalkenyl, each of which is optionally substituted with X¹ and further optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{c}; and
C₆₋₁₀ aryl optionally substituted with X¹ and further optionally substituted with from 1-4 R^{c};
X¹ is —(X²)ₘ-L¹-R⁵, wherein: m is 0 or 1;
X² is selected from the group consisting of:
—O—, —N(R^{N})—, or —S(O)₀₋₂;

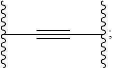 [SMILES: *C#C*]

—C₂₋₆ alkenylene optionally substituted with from 1-3 R^{a};
—C(═O)O—*, —C(═O)N(R^{N})—*, or —S(O)₁₋₂N(R^{N})—*.
—OC(═O)—*, —N(R^{N})C(═O)—*, or —N(R^{N})S(O)₁₋₂—*; and
—OC(═O)N(R^{N})—*, —N(R^{N})C(═O)O—*, —N(R^{N})C(═O)N(R^{N})—*, or —N(R^{N})S(O)₁₋₂N(R^{N})—*,
wherein the asterisk represents point of attachment to L¹;
L¹ is selected from the group consisting of: a bond and C₁₋₁₀ alkylene optionally substituted with from 1-6 R^{a};
R⁵ is selected from the group consisting of:
H;
halo;
—OH;
—NR^{e}R^{f};
—C₁₋₆ alkoxy or —S(O)₀₋₂(C₁₋₆ alkyl), each optionally substituted with from 1-6 R^{a};
—R^{g};
-L⁵-R^{g};
—R^{g2}-R^{W} or —R^{g2}-R^{Y}; and
-L⁵-R^{g2}-R^{W} or -L⁵-R^{g2}-R^{Y};
provided that:
when L¹ is a bond, then R⁵ is selected from the group consisting of: H, —R^{g}, —R^{g2}—R^{W}, and —R^{g2}-R^{Y}; and
X¹ is other than H, —OH, or NH₂;
L⁵ is selected from the group consisting of: —O—, —S(O)₀₋₂, —NH—, and —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is selected from the group consisting of:
C₂₋₆ alkenyl; C₂₋₆ alkynyl; or C₃₋₁₀ alkenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
bicyclo[x.y.0]cycloalkyl optionally substituted with from 1-2 R^{c}, wherein x is 1 or 2; and y is an integer from 1 to 6;
R^{Y} is selected from the group consisting of —R^{g} and -(L)_{g}-R^{g};
each of R^{1c}, R^{2a}, R^{2b}, R^{3a}, and R^{3b} is independently selected from the group consisting of H; halo; —OH; —C(O)OH or —C(O)NH₂; —CN; —R^{b}; -L^{b}-R^{b}; —NR^{e}R^{f}; —R^{g}; -(L^{g})_{g}-R^{g}; -(L^{g})_{g}-R^{W}; -(L)_{g}-R^{g2}-R^{W}; and —C₁₋₆ alkoxy or —C₁₋₆ thioalkoxy, each optionally substituted with from 1-6 R^{a}; provided that R^{1c} is other than halo, —CN, or —C(O)OH; or
two of variables R^{1c}, R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W}; or
one of R^{2a} and R^{2b} and one of R^{3a} and R^{3b} combine to form a double bond between the Ring B atoms to which each is attached;
Ring A is R^{g};
R⁴ and R⁷ are independently H or R^{d};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″, —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}, C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group;
each occurrence of R′ and R″ is independently selected from the group consisting of: H; —OH; and C₁₋₄ alkyl; and
each occurrence of R^{N} is independently H, C₁₋₃ alkyl, or C₃₋₆ cycloalkyl,
provided that one or more of the following applies:
when R^{2a} and R^{2b} are H or methyl; R^{3a} and R^{3b} are H; Ring C is

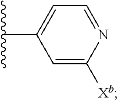

and X^{b} is H, methyl, NH₂, NHC(═O)Me, NHC(═O)iPr, NHC(═O)NHEt,

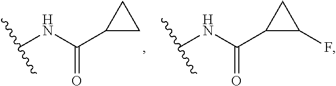

then Ring A is other than unsubstituted phenyl;
when R^{2a}, R^{2b}, R^{3a}, and R^{3b} are each H; Ring C is

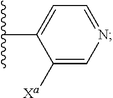

and X^{a} is methyl or F, then Ring A is other than unsubstituted phenyl;
when R^{1c}, R^{2a}, R^{2b}, R^{3a}, and R^{3b} are each H; Ring C is

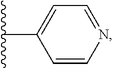 [SMILES: *c1ccncc1]

then Ring A is other than 4-fluorophenyl; and
the compound is other than:

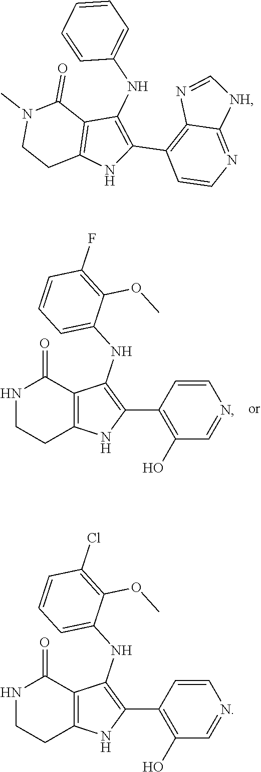 [SMILES: CN1CCc2[nH]c(-c3ccnc4[nH]cnc34)c(Nc3ccccc3)c2C1=O ; COc1c(F)cccc1Nc1c(-c2ccncc2O)[nH]c2c1C(=O)NCC2 ; "or" ; COc1c(Cl)cccc1Nc1c(-c2ccncc2O)[nH]c2c1C(=O)NCC2]

Also provided herein is a pharmaceutical composition comprising a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, and a pharmaceutically acceptable carrier.

Provided herein is a method for treating cancer in a subject in need thereof, the method comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

Also provided herein is a method for treating cancer in a subject in need thereof, the method comprising (a) determining that the cancer is associated with a dysregulation of an EGFR gene, an EGFR kinase, or expression or activity or level of any of the same; and (b) administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

Provided herein is a method of treating an EGFR-associated disease or disorder in a subject, the method comprising administering to a subject identified or diagnosed as having an EGFR-associated disease or disorder a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

This disclosure also provides a method of treating an EGFR-associated disease or disorder in a subject, the method comprising: determining that the cancer in the subject is an EGFR-associated disease or disorder; and administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein. Further provided herein is a method of treating an EGFR-associated cancer in a subject, the method comprising administering to a subject identified or diagnosed as having an EGFR-associated cancer a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

This disclosure also provides a method of treating an EGFR-associated cancer in a subject, the method comprising: determining that the cancer in the subject is an EGFR-associated cancer; and administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

Provided herein is a method of treating a subject, the method comprising administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein, to a subject having a clinical record that indicates that the subject has a dysregulation of an EGFR gene, an EGFR kinase, or expression or activity or level of any of the same.

Also provided herein is a method of treating a subject having a cancer, wherein the method comprises.
(a) administering one or more doses of a first EGFR inhibitor to the subject for a period of time;
(b) after (a), determining whether a cancer cell in a sample obtained from the subject has at least one EGFR inhibitor resistance mutation that confers increased resistance to a cancer cell or tumor to treatment with the first EGFR inhibitor of step (a); and
(c) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor of step (a); or
(d) administering additional doses of the first EGFR inhibitor of step (a) to the subject if the subject has not been determined to have a cancer cell that has at least one EGFR inhibitor resistance mutation that confers increased resistance to a cancer cell or tumor to treatment with the first EGFR inhibitor of step (a).

Further provided herein is a method of treating a subject having a cancer, wherein the method comprises:
(a) determining whether a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor that was previously administered to the subject; or
(c) administering additional doses of the first EGFR inhibitor to the subject if the subject has not been determined to have a cancer cell that has at least one EGFR inhibitor resistance mutation that confers increased resistance to a cancer cell or tumor to treatment with the first EGFR inhibitor previously administered to the subject.

Also provided herein is a method of treating a subject having a cancer, wherein the method comprises:
(a) determining that a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first EGFR inhibitor does not have one or more EGFR inhibitor resistance mutations that confer increased resistance to a cancer cell or tumor to treatment with the first EGFR inhibitor that was previously administered to the subject; and
(b) administering additional doses of the first EGFR inhibitor to the subject.

This disclosure also provides a method for inhibiting EGFR in a mammalian cell, the method comprising contacting the mammalian cell with an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

Also provided herein is a method for treating cancer in a subject in need thereof, the method comprising (a) determining that the cancer is associated with a dysregulation of a HER2 gene, a HER2 kinase, or expression or activity or level of any of the same; and (b) administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

Further provided herein is a method of treating a HER2-associated cancer in a subject, the method comprising administering to a subject identified or diagnosed as having a HER2-associated cancer a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

This disclosure also provides a method of treating a HER2-associated cancer in a subject, the method comprising: determining that the cancer in the subject is a HER2-associated cancer; and administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

Provided herein is a method of treating a subject having a cancer, the method comprising administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein, to a subject having a clinical record that indicates that the subject has a dysregulation of a HER2 gene, a HER2 kinase, or expression or activity or level of any of the same.

Also provided herein is a method of treating a subject having a cancer, wherein the method comprises:
(a) administering one or more doses of a first HER2 inhibitor to the subject for a period of time;
(b) after (a), determining whether a cancer cell in a sample obtained from the subject has at least one HER2 inhibitor resistance mutation that confers increased resistance to a cancer cell or tumor to treatment with the first HER2 inhibitor of step (a); and
(c) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject if the subject has been determined to have a cancer cell that has at least one HER2 inhibitor resistance mutation that confers increased resistance to a cancer cell or tumor to treatment with the first HER2 inhibitor of step (a); or
(d) administering additional doses of the first HER2 inhibitor of step (a) to the subject if the subject has not been determined to have a cancer cell that has at least one HER2 inhibitor resistance mutation that confers increased resistance to a cancer cell or tumor to treatment with the first HER2 inhibitor of step (a).

Further provided herein is a method of treating a subject having a cancer, wherein the method comprises:
(a) determining whether a cancer cell in a sample obtained from a subject having a cancer and previously administered one or more doses of a first HER2 inhibitor has one or more HER2 inhibitor resistance mutations that confer increased resistance to a cancer cell or tumor to treatment with the first HER2 inhibitor that was previously administered to the subject; and
(b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject if the subject has been determined to have a cancer cell that has at least one HER2 inhibitor resistance mutation that confers increased resistance to a cancer cell or tumor to treatment with the first HER2 inhibitor that was previously administered to the subject; or
(c) administering additional doses of the first HER2 inhibitor to the subject if the subject has not been determined to have a cancer cell that has at least one HER2 inhibitor resistance mutation that confers increased resistance to a cancer cell or tumor to treatment with the first HER2 inhibitor previously administered to the subject.

Also provided herein is a method of treating a subject having a cancer, wherein the method comprises:
(a) determining that a cancer cell in a sample obtained from a subject having a cancer and previously administered one or more doses of a first HER2 inhibitor has one or more HER2 inhibitor resistance mutations that confer increased resistance to a cancer cell or tumor to treatment with the first HER2 inhibitor that was previously administered to the subject; and
(b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first HER2 inhibitor does not have one or more HER2 inhibitor resistance mutations that confer increased resistance to a cancer cell or tumor to treatment with the first HER2 inhibitor that was previously administered to the subject; and
(b) administering additional doses of the first HER2 inhibitor to the subject.

This disclosure also provides a method for inhibiting HER2 in a mammalian cell, the method comprising contacting the mammalian cell with an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

Also provided herein is a method for treating cancer in a subject in need thereof, the method comprising (a) determining that the cancer is associated with a dysregulation of an EGFR gene, an EGFR kinase, or expression or activity or level of any of the same and that the cancer is associated with a dysregulation of a HER2 gene, a HER2 kinase, or expression or activity or level of any of the same; and (b) administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

Further provided herein is a method of treating an EGFR-associated and HER2-associated cancer in a subject, the method comprising administering to a subject identified or diagnosed as having an EGFR-associated and a HER2-associated cancer a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

This disclosure also provides a method of treating a an EGFR-associated and HER2-associated cancer in a subject, the method comprising: determining that the cancer in the subject is an EGFR-associated and a HER2-associated cancer; and administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein.

Provided herein is a method of treating a subject, the method comprising administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition as provided herein, to a subject having a clinical record that indicates that the subject has a dysregulation of an EGFR gene, an EGFR kinase, or expression or activity or level of any of the same and a dysregulation of a HER2 gene, a HER2 kinase, or expression or activity or level of any of the same.

This disclosure also provides a method for inhibiting EGFR and HER2 in a mammalian cell, the method comprising contacting the mammalian cell with an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

In addition to the above, provided herein is a method for inhibiting a BUB (budding uninhibited by benzimidazole, BUB1-3) kinase. In some embodiments, the methods provided herein include methods for inhibiting BUB11. For example, a method for inhibiting BUB1 in a mammalian cell, the method comprising contacting the mammalian cell with an effective amount of a compound of Formula (I) (e.g., Formula (I-a), (I-b), (I-c), (I-d), (I-e), (I-f), (I-g), (I-h), (I-i), (I-j), or (i-k)), or a pharmaceutically acceptable salt thereof.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.: Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, in-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as or

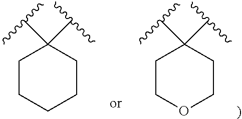

or on different ring atoms (e.g., ring carbon and/or nitrogen atoms (e.g., vicinal ring carbon and/or nitrogen atoms))

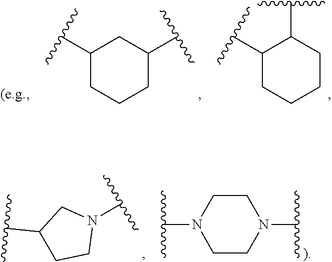 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

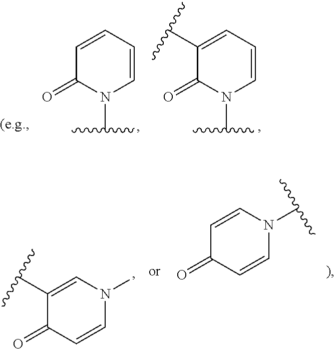

pyrimidone

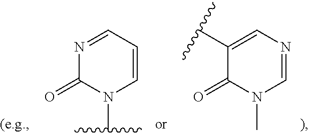

pyridazinone

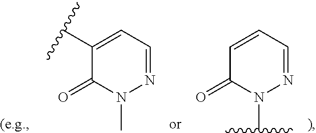

pyrazinone

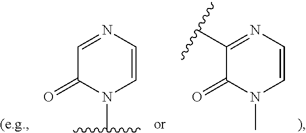

and imidazolone

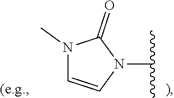 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

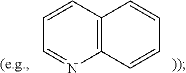

(ii) a single ring atom (spiro-fused ring systems)

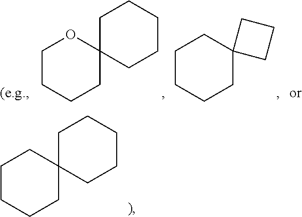

or (iii) a contiguous array of ring atoms (bridged ring systems having all bridge lengths >0)

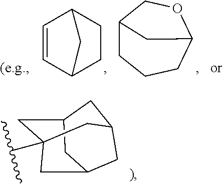

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

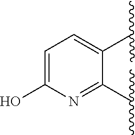 [SMILES: *c1ccc(O)nc1*]

encompasses the tautomeric form containing the moiety:

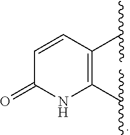 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

The details of one or more embodiments of the invention are set forth in the accompanying drawings and the description below. Other features and advantages of the invention will be apparent from the description and drawings, and from the claims.

### DETAILED DESCRIPTION

This disclosure provides chemical entities (e.g., a compound or a pharmaceutically acceptable salt, and/or hydrate, and/or cocrystal, and/or drug combination of the compound) that inhibit epidermal growth factor receptor (EGFR, ERBB1) and/or Human epidermal growth factor receptor 2 (HER2, ERBB2). These chemical entities are useful, e.g., for treating a condition, disease or disorder in which increased (e.g., excessive) EGFR and/or HER2 activation contributes to the pathology and/or symptoms and/or progression of the condition, disease or disorder (e.g., cancer) in a subject (e.g., a human). In some embodiments, the chemical entities provided herein can inhibit an EGFR kinase and/or a HER2 kinase that has an exon 20 mutation (e.g., any of the exon 20 mutations described herein). Exon 20 mutations can confer intrinsic resistance to EGFR and/or HER2 inhibitors, and there are currently only limited targeted therapies that have been approved for subjects with these mutations. This disclosure also provides compositions containing the chemical entities provided herein as well as methods of using and making the same.

### Formulae (I) Compounds

In one aspect, this disclosure features compounds of Formula (I):

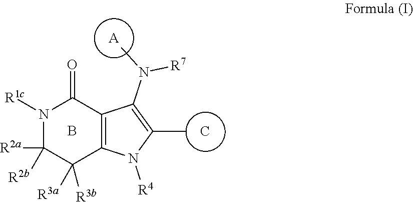

or a pharmaceutically acceptable salt thereof, wherein:

Ring C is selected from the group consisting of:

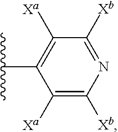

wherein:
each X^{b} is independently X¹, R^{c}, or H, and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂, —C(═O)(C₁₋₁₀ alkyl); —C(═O)O(C₁₋₄ alkyl), —C(═O)OH; —C(═O)NR′R″; and —SF₅;
2-pyridyl or 3-pyridyl, each optionally substituted with X¹ and further optionally substituted with from 1-4 R^{c};
2-pyridonyl or 4-pyridonyl, each optionally substituted with X¹ and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 substituents independently selected from the group consisting of oxo and R^{c};
C₃₋₁₀ cycloalkyl or C₃₋₁₀ cycloalkenyl, each of which is optionally substituted with X¹ and further optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{c}; and
C₆₋₁₀ aryl optionally substituted with X¹ and further optionally substituted with from 1-4 R^{c};
X¹ is —(X²)ₘ-L¹-R⁵, wherein: m is 0 or 1;
X² is selected from the group consisting of:
—O—, —N(R^{N})—, or —S(O)₀₋₂;

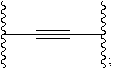 [SMILES: *C#C*]

C₂₋₆ alkenylene optionally substituted with from 1-3 R^{a};
—C(═O)O—*, —C(═O)N(R^{N})—*, or S(O)₁₋₂N(R^{N})—*;
—OC(═O)—*, —N(R^{N})C(═O)—*, or —N(R^{N})S(O)₁₋₂—*; and
—OC(═O)N(R^{N})—*, —N(R^{N})C(═O)O—*, —N(R^{N})C(═O)N(R^{N})—*, or —N(R^{N})S(O)₁₋₂N(R^{N})—*,
wherein the asterisk represents point of attachment to L¹;
L¹ is selected from the group consisting of: a bond and C₁₋₁₀ alkylene optionally substituted with from 1-6 R^{a};
R⁵ is selected from the group consisting of:
H;
halo;
—OH;
—NR^{e}R^{f};
—C₁₋₆ alkoxy or —S(O)₀₋₂(C₁₋₆ alkyl), each optionally substituted with from 1-6 R^{a};
—R^{g};
-L⁵-R^{g};
—R^{g2}-R^{W} or —R^{g2}-R^{Y}; and
-L⁵-R^{g2}-R^{W} or -L⁵-R^{g2}-R^{Y};
provided that:
when L¹ is a bond, then R⁵ is selected from the group consisting of: H, —R^{g}, —R^{g2}—R^{W}, and —R^{g2}-R^{Y}; and
X¹ is other than H, —OH, or NH₂;
L⁵ is selected from the group consisting of: —O—, —S(O)₀₋₂, —NH, and —N(R^{d})—.
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is selected from the group consisting of:
C₂₋₆ alkenyl; C₂₋₆ alkynyl; or C₃₋₁₀ alkenyl, each of which is optionally substituted with from 1-3 R^{a} and further optionally substituted with R&, wherein W is attached to L^{W} via an sp² or sp hybridized carbon atom, thereby providing an α, β-unsaturated system; and
bicyclo[x.y.0]cycloalkyl optionally substituted with from 1-2 R^{c}, wherein x is 1 or 2; and y is an integer from 1 to 6;
R^{Y} is selected from the group consisting of: —R^{g} and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —NR^{e}R^{f}; —R^{g}; -(L^{g})_{g}-R^{g}; and —C₁₋₆ alkoxy or —C₁₋₆ thioalkoxy, each optionally substituted with from 1-6 R^{a}; provided that R^{1c} is other than halo, —CN, or —C(O)OH; or
or two of variables R^{1c}, R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W};
Ring A is R^{g};
R⁴ and R⁷ are independently H or R^{d};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl), —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group;
each occurrence of R′ and R″ is independently selected from the group consisting of: H; —OH; and C₁₋₄ alkyl; and
each occurrence of R^{N} is independently H, C₁₋₃ alkyl, or C₃₋₆ cycloalkyl.

In one aspect, this disclosure features compounds of Formula (I):

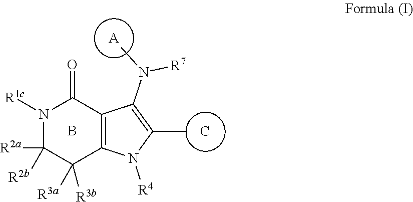

or a pharmaceutically acceptable salt thereof, wherein:

Ring C is selected from the group consisting of:

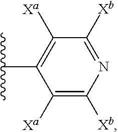

wherein:
each X^{b} is independently X¹, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X¹ and further optionally substituted with from 1-4 R^{c};
2-pyridonyl or 4-pyridonyl, each optionally substituted with X¹ and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 substituents independently selected from the group consisting of oxo and R^{c};
C₃₋₁₀ cycloalkyl or C₃₋₁₀ cycloalkenyl, each of which is optionally substituted with X¹ and further optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{c}; and
C₆₋₁₀ aryl optionally substituted with X¹ and further optionally substituted with from 1-4 R^{c};
X¹ is —(X²)ₘ-L¹-R⁵, wherein: m is 0 or 1;
X² is selected from the group consisting of:
—O—, —N(R^{N})—, or —S(O)₀₋₂;

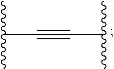 [SMILES: *C#C*]

C₂₋₆ alkenylene optionally substituted with from 1-3 R^{a};
—C(═O)O—*, —C(═O)N(R^{N})—*, or —S(O)₁₋₂N(R^{N})—*.
—OC(═O)—*, —N(R^{N})C(═O)—*, or —N(R^{N})S(O)₁₋₂—*; and
—OC(═O)N(R^{N})—*, —N(R^{N})C(═O)O—*, —N(R^{N})C(═O)N(R^{N})—*, or —N(R^{N})S(O)₁₋₂N(R^{N})—*,
wherein the asterisk represents point of attachment to L¹;
L¹ is selected from the group consisting of: a bond and C₁₋₁₀ alkylene optionally substituted with from 1-6 R^{a};
R⁵ is selected from the group consisting of:
H;
halo;
—OH;
—NR^{e}R^{f};
—C₁₋₆ alkoxy or —S(O)₀₋₂(C₁₋₆ alkyl), each optionally substituted with from 1-6 R^{a};
—R^{g};
-L⁵-R^{g};
—R^{g2}-R^{W} or —R^{g2}-R^{Y}; and
-L⁵-R^{g2}-R^{W} or -L⁵-R^{g2}-R^{Y};
provided that:
when L¹ is a bond, then R⁵ is selected from the group consisting of: H, —R^{g}, —R^{g2}—R^{W}, and —R^{g2}-R^{Y}; and
X¹ is other than H, —OH, or NH₂;
L⁵ is selected from the group consisting of: —O—, —S(O)₀₋₂, —NH, and —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ alkenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{Y} is selected from the group consisting of: —R^{g} and -(L^{g})_{g}-R^{g};
each of R^{1c}, R^{2a}, R^{2b}, R^{3a}, and R^{3b} is independently selected from the group consisting of H; halo; —OH; —C(O)OH or —C(O)NH₂; —CN; —R^{b}; -L^{b}-R^{b}; —NR^{e}R^{f}; —R^{g}; -(L^{g})_{g}-R^{g}; and —C₁₋₆ alkoxy or —C₁₋₆ thioalkoxy, each optionally substituted with from 1-6 R^{a}; provided that R^{1c} is other than halo, —CN, or —C(O)OH; or
or two of variables R^{1c}, R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W};
Ring A is R^{g};
R⁴ and R⁷ are independently H or R^{d};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl), —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group;
each occurrence of R′ and R″ is independently selected from the group consisting of: H; —OH; and C₁₋₄ alkyl; and
each occurrence of R^{N} is independently H, C₁₋₃ alkyl, or C₃₋₆ cycloalkyl.

In some embodiments, it is provided that one or more of the following applies:
when R^{2a} and R^{2b} are H or methyl; R^{3a} and R^{3b} are H; Ring C is

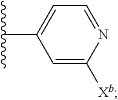

and X^{b} is H, methyl, NH₂, NHC(═O)Me, NHC(═O)iPr, NHC(═O)NHEt,

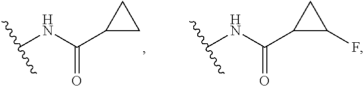 [SMILES: *NC(=O)C1CC1 ; *NC(=O)C1CC1F]

then Ring A is other than unsubstituted phenyl;
when R^{2a}, R^{2b}, R^{3a}, and R^{3b} are each H; Ring C is

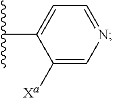

and X^{a} is methyl or F, then Ring A is other than unsubstituted phenyl;
when R^{1c}, R^{2a}, R^{2b}, R^{3a}, and R^{3b} are each H; Ring C is

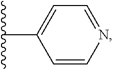 [SMILES: *c1ccncc1]

then Ring A is other than 4-fluorophenyl; and
the compound is other than:

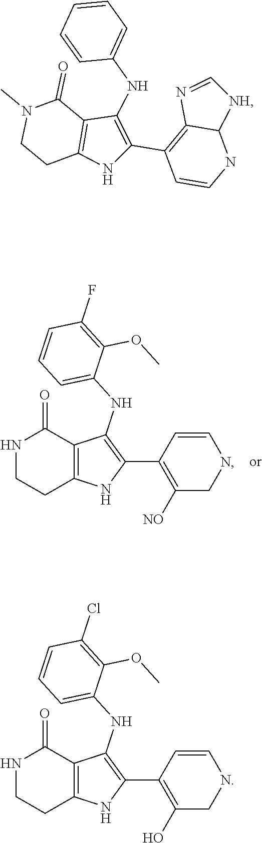

In one aspect, this disclosure features a compound of Formula (I):

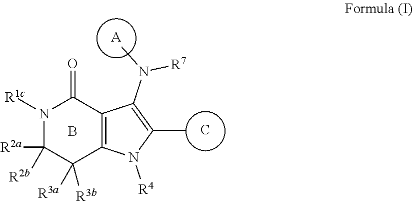

or a pharmaceutically acceptable salt thereof, wherein:

Ring C is selected from the group consisting of:

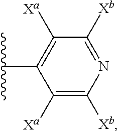

wherein:
each X^{b} is independently X¹, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH, —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X¹ and further optionally substituted with from 1-4 R^{c};
2-pyridonyl or 4-pyridonyl, each optionally substituted with X¹ and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 substituents independently selected from the group consisting of oxo and R^{c};
C₃₋₁₀ cycloalkyl or C₃₋₁₀ cycloalkenyl, each of which is optionally substituted with X¹ and further optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{c}; and
C₆₋₁₀ aryl optionally substituted with X¹ and further optionally substituted with from 1-4 R^{c};
X¹ is —(X²)ₘ-L¹-R⁵, wherein: m is 0 or 1;
X² is selected from the group consisting of:
—O—, —N(R^{N})—, or —S(O)₀₋₂;

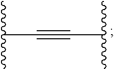 [SMILES: *C#C*]

—C₂₋₆ alkenylene optionally substituted with from 1-3 R^{a};
—C(═O)O—*, —C(═O)N(R^{N})—*, or —S(O)₁₋₂N(R^{N})—*;
—OC(═O)—*, —N(R^{N})C(═O)—*, or —N(R^{N})S(O)₁₋₂—*; and
—OC(═O)N(R^{N})—*, —N(R^{N})C(═O)O—*, —N(R^{N})C(═O)N(R^{N})—*, or —N(R^{N})S(O)₁₋₂N(R^{N})—*,
wherein the asterisk represents point of attachment to L¹;
L¹ is selected from the group consisting of: a bond and C₁₋₁₀ alkylene optionally substituted with from 1-6 R^{a};
R⁵ is selected from the group consisting of:
H;
halo;
—OH;
—NR^{e}R^{f};
—C₁₋₆ alkoxy or —S(O)₀₋₂(C₁₋₆ alkyl), each optionally substituted with from 1-6 R^{a};
—R^{g};
-L⁵-R^{g};
—R^{g2}-R^{W} or —R^{g2}-R^{Y}; and
-L⁵-R^{g2}-R^{W} or -L⁵-R^{g2}-R^{Y};
provided that:
when L¹ is a bond, then R⁵ is selected from the group consisting of: H, —R^{g}, —R^{g2}—R^{W}, and —R^{g2}-R^{Y}; and
X¹ is other than H, —OH, or NH₂;
L⁵ is selected from the group consisting of: —O—, —S(O)₀₋₂, —NH—, and —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is selected from the group consisting of:
C₂₋₆ alkenyl; C₂₋₆ alkynyl; or C₃₋₁₀ alkenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
bicyclo[x.y.0]cycloalkyl optionally substituted with from 1-2 R^{c}, wherein x is 1 or 2; and y is an integer from 1 to 6;
R^{Y} is selected from the group consisting of: —R^{g} and -(L^{g})_{g}-R^{g};
each of R^{1c}, R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —NR^{e}R^{f}; —R^{g}; -(L^{g})_{g}-R^{f}; -(L^{f})_{g}-R^{W}; -(L^{g})_{g}-R^{g2}-R^{W}; and —C₁₋₆ alkoxy or —C₁₋₆ thioalkoxy, each optionally substituted with from 1-6 R^{a}; provided that R^{1c} is other than halo, —CN, or —C(O)OH; or
two of variables R^{1c}, R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W}; or
one of R^{2a} and R^{2b} and one of R^{3a} and R^{3b} combine to form a double bond between the Ring B atoms to which each is attached;
Ring A is R^{g};
R⁴ and R⁷ are independently H or R^{d};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₄ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group;
each occurrence of R′ and R″ is independently selected from the group consisting of: H; —OH, and C₁₋₄ alkyl; and
each occurrence of R^{N} is independently H, C₁₋₃ alkyl, or C₃₋₆ cycloalkyl,
provided that one or more of the following applies:
when R^{2a} and R^{2b} are H or methyl; R^{3a} and R^{3b} are H; Ring C is

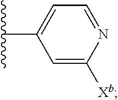

and X^{b} is H, methyl, NH₂, NHC(═O)Me, NHC(═O)iPr, NHC(═O)NHEt,

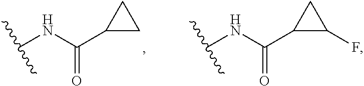 [SMILES: *NC(=O)C1CC1 ; *NC(=O)C1CC1F]

then Ring A is other than unsubstituted phenyl;
when R^{2a}a, R^{2b}, R^{3a}, and R^{3b} are each H; Ring C is

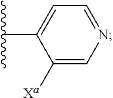

and X^{a} is methyl or F, then Ring A is other than unsubstituted phenyl:
when R^{1c}, R^{2a}, R^{2b}, R^{3a}, and R^{3b} are each H; Ring C is

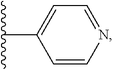 [SMILES: *c1ccncc1]

then Ring A is other than 4-fluorophenyl; and
the compound is other than:

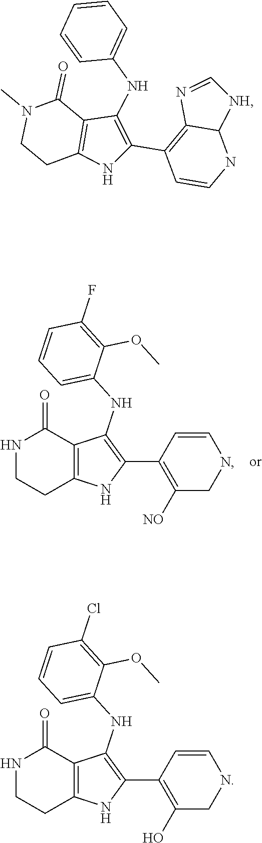

Variable Ring C

In some embodiments, Ring C is heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{cA}, wherein each R^{cA} is an independently selected R^{c}.

In certain of the foregoing embodiments, Ring C is heteroaryl including 6 ring atoms, wherein from 2-3 ring atoms are heteroatoms, each independently selected from the group consisting of N, N(H), and N(R^{d}), and wherein the heteroaryl is optionally substituted with from 1-3 R^{cA}, wherein each R^{cA} is an independently selected R^{c}.

In certain of these embodiments, Ring C is pyrimidyl optionally substituted with from 1-3 R^{cA}, such as pyrimidyl substituted with from 1-2 R^{cA}, wherein each R^{cA} is an independently selected R^{c}.

In certain embodiments, Ring C is

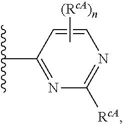

wherein each R^{cA} is an independently selected R^{c}; and n is 0, 1, or 2.

As a non-limiting example of the foregoing embodiments, Ring C can be

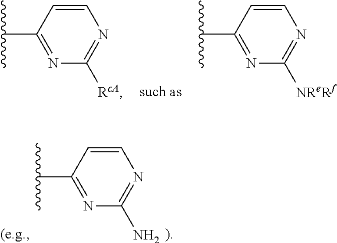

In certain foregoing embodiments, n is 0 and R^{cA} is C₁₋₁₀ alkyl optionally substituted with from 1-6 independently selected R^{a}, e.g., C₁₋₃ alkyl optionally substituted with from 1-3 independently selected halo.

As a non-limiting example, Ring C can be

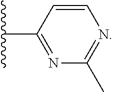 [SMILES: *c1ccnc(C)n1]

As another non-limiting example, Ring C can be

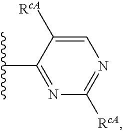

such as

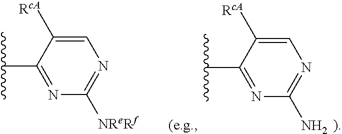

As another non-limiting example, Ring C can be

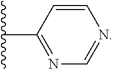 [SMILES: *c1ccncn1]

In certain embodiments, Ring C is triazinyl optionally substituted with from 1-2 R^{cA}, wherein each R^{cA} is an independently selected R^{c}. For example, Ring C can be

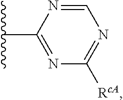

such as

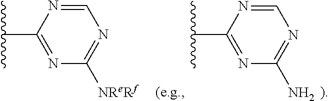

In certain embodiments, Ring C is heteroaryl including 6 ring atoms, wherein from 2-3 ring atoms are heteroatoms, each independently selected from the group consisting of N, N(H), and N(R^{d}), and wherein the heteroaryl is substituted with X¹ and further optionally substituted with from 1-2 R^{cA}, wherein each R^{cA} is an independently selected R^{c}.

In certain of the foregoing embodiments, Ring C is pyrimidyl substituted with X¹ and further optionally substituted with from 1-2 R^{cA}, wherein each R^{cA} is an independently selected R^{c}.

In certain of these embodiments, Ring C is

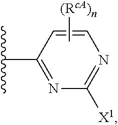

wherein each R^{cA} is an independently selected R^{c}; and n is 0, 1, or 2.

As a non-limiting example of the foregoing embodiments, Ring C can be

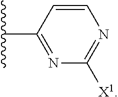

In certain embodiments, Ring C is

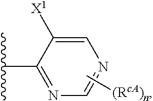

wherein n is 0, 1, or 2; and each R^{cA} is an independently selected R^{c}. As a non-limiting example of the foregoing embodiments, Ring C can be

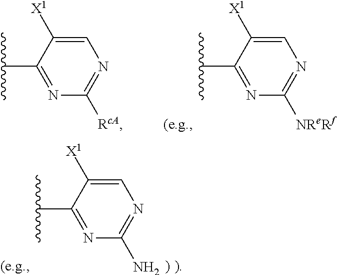

In some embodiments, Ring C is bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{cA}, wherein each R^{cA} is an independently selected R^{c}.

In certain of the foregoing embodiments, Ring C is bicyclic heteroaryl including 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{cA}, wherein each R^{cA} is an independently selected R^{c}.

In certain of the foregoing embodiments, Ring C is bicyclic heteroaryl including 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

In certain of these embodiments, Ring C is connected to

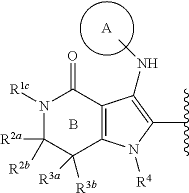

via a 6-membered ring.

In certain embodiments, Ring C is

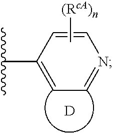

Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n is 0, 1, or 2; and each R^{cA} is an independently selected R^{c}.

In certain of these embodiments, Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

As non-limiting examples of the foregoing embodiments, Ring C can be selected from the group consisting of:

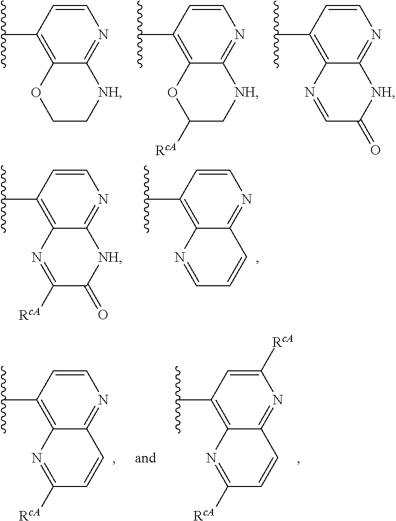

each further optionally substituted with R^{cA}, wherein each R^{cA} is an independently selected R^{c}. For example, Ring C can be

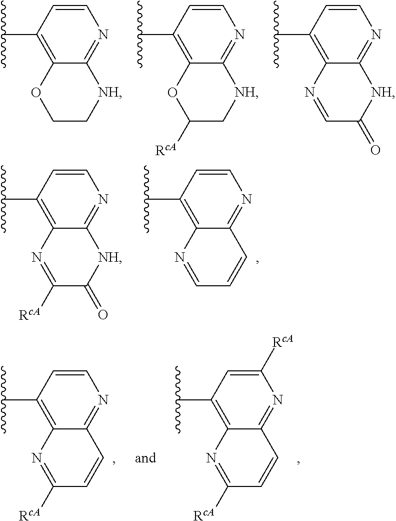

As non-limiting examples of the foregoing embodiments, Ring C is selected from the group consisting of

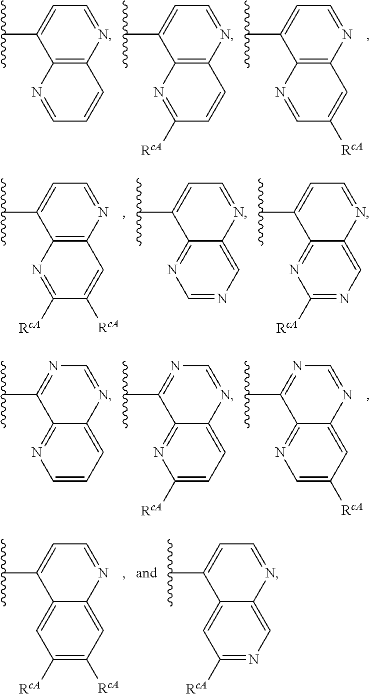

each further optionally substituted with R^{cA}, wherein each R^{cA} is an independently selected R^{c}.

In certain of these embodiments, Ring C is

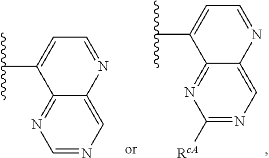

wherein R^{cA} is an independently selected R^{c}.

In certain of these embodiments, Ring C is

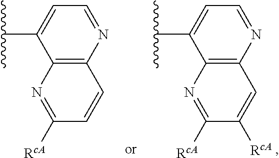

wherein each R^{cA} is an independently selected R^{c}.

In certain of these embodiments, Ring C is

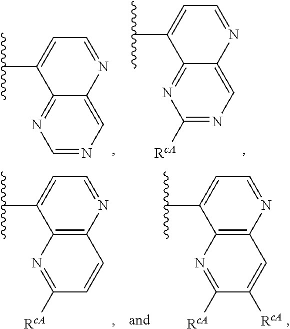

wherein each occurrence of R^{cA} is independently selected from the group consisting of: halo, NR^{e}R^{f}, C₁₋₄ alkoxy, C₁₋₄ haloalkoxy, C₁₋₃ alkyl, C₁₋₃ alkyl substituted with from 1-3 independently selected halo, C₁₋₃ alkyl substituted with C₁₋₄ alkoxy, and C₁₋₄ alkoxy substituted with C₁₋₄ alkoxy, and wherein each occurrence of R^{cA} is independently selected from the group consisting of: C₁₋₄ alkoxy; C₁₋₄ haloalkoxy: C₁₋₃ alkyl; and C₁₋₃ alkyl substituted with from 1-3 independently selected halo.

In certain embodiments (when Ring C is

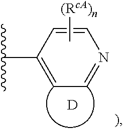

Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

As non-limiting examples of the foregoing embodiments, Ring C can be selected from the group consisting of:

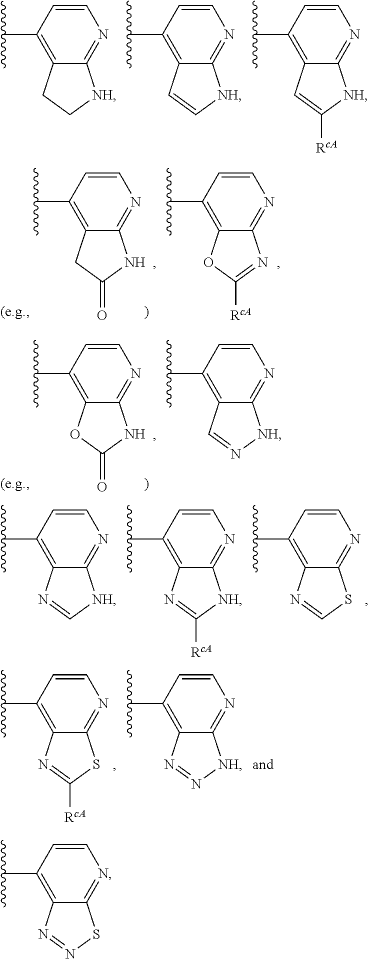

each further optionally substituted with R^{cA}, wherein each R^{cA} is an independently selected R^{c}. For example, Ring C can be

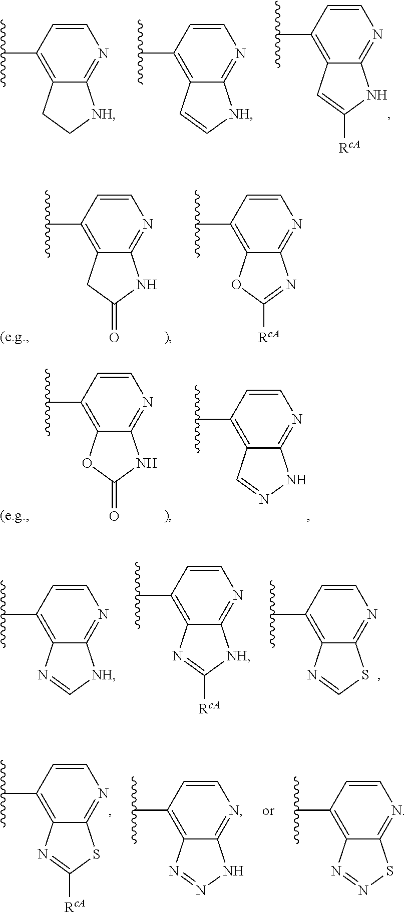

As another non-limiting example, Ring C can be

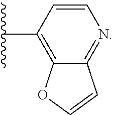 [SMILES: *c1ccnc2ccoc12]

In certain embodiments, Ring C is

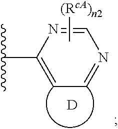

Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n2 is 0 or 1; and each R^{cA} is an independently selected R^{c}.

In certain of these embodiments, Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

As non-limiting examples of the foregoing embodiments, Ring C can be selected from the group consisting of:

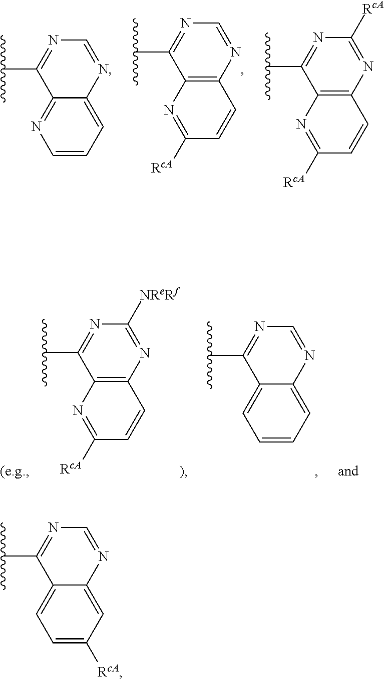

each further optionally substituted with R^{cA}, wherein each R^{cA} is an independently selected R^{c}. For example, Ring C can be

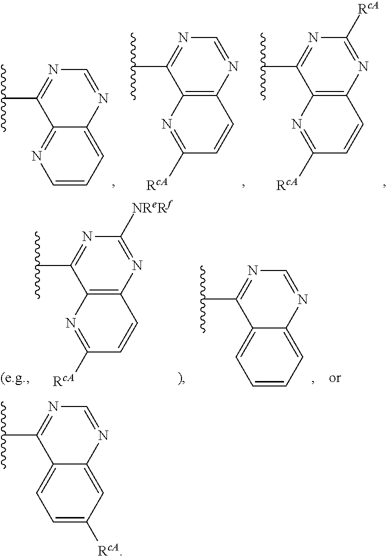

In certain embodiments (when Ring C is

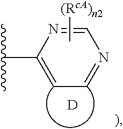

Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

As non-limiting examples of the foregoing embodiments, Ring C can be selected from the group consisting of:

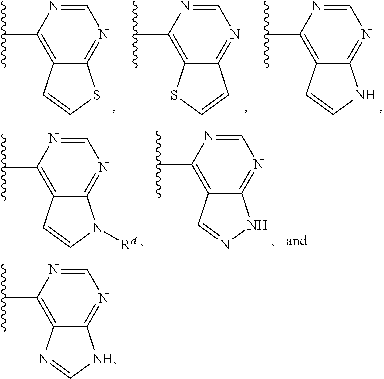

each further optionally substituted with R^{cA}, wherein each R^{cA} is an independently selected R^{c}.

In certain embodiments, Ring C is selected from the group consisting of:

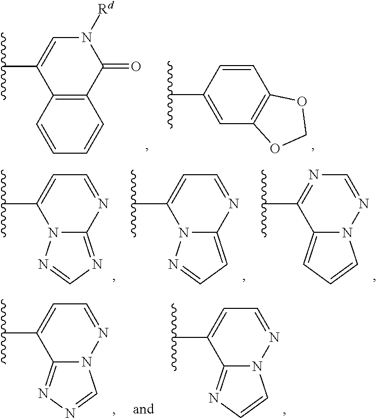

each further optionally substituted with R^{cA}, wherein each R^{cA} is an independently selected R^{c}.

In certain embodiments (when Ring C is bicyclic heteroaryl including 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}), Ring C is connected to

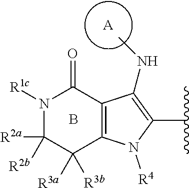

via a 5-membered ring.

As non-limiting examples of the foregoing embodiments, Ring C can be selected from the group consisting of:

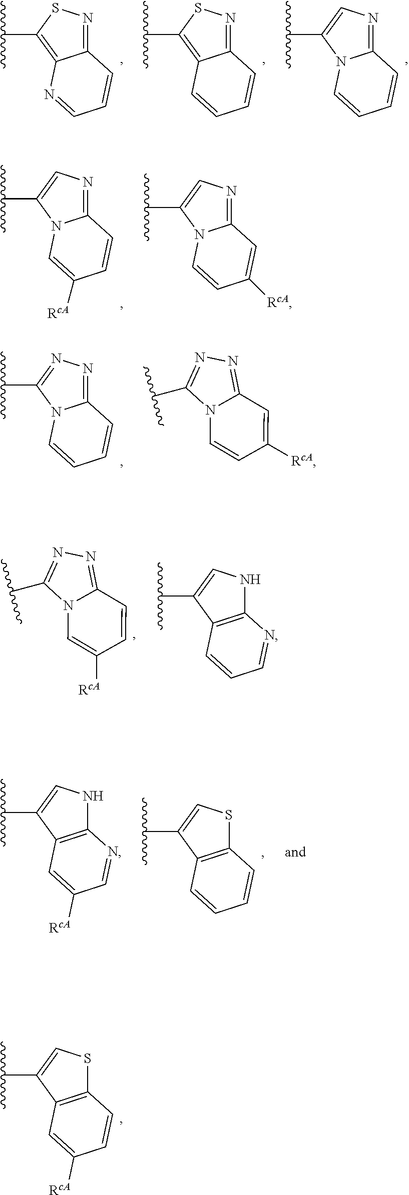

each further optionally substituted with R^{cA}, wherein each R^{cA} is an independently selected R^{c}.

In certain embodiments, Ring C is bicyclic heteroaryl including 9-10 ring atoms, wherein from 1-4 ring atoms are heteroatoms, each independently selected from the group consisting of N, N(H), N(R^{d}), O, and S(O)₀₋₂, and wherein the heteroaryl is substituted with X¹ and further optionally substituted with from 1-4 R^{cA}, wherein each R^{cA} is an independently selected R^{c}.

In certain of these embodiments, Ring C is

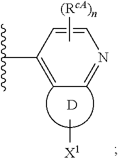

Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n is 0, 1, or 2; and each R^{cA} is an independently selected R^{c}.

In certain of the foregoing embodiments, Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

As non-limiting examples of the foregoing embodiments, Ring C can be selected from the group consisting of:

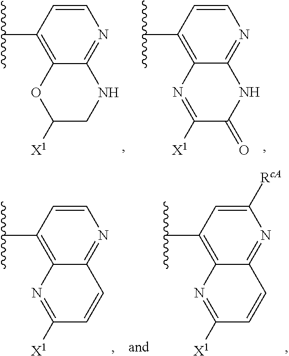

each further optionally substituted with R^{cA}, wherein each R^{cA} is an independently selected R^{c}. For example, Ring C can be

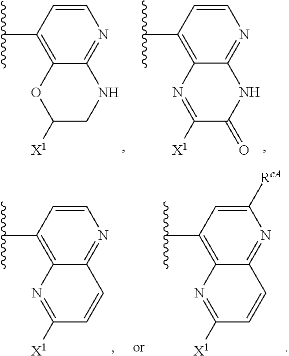

As non-limiting examples of the foregoing embodiments, Ring C is

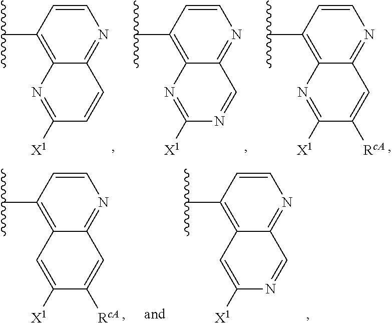

each of which is further optionally substituted with from 1-2 R^{cA}, wherein each R^{cA} is an independently selected R^{c}.

In certain of these embodiments, Ring C is

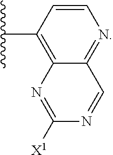

In certain of these embodiments, Ring C is

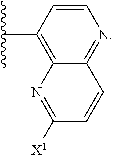

In certain of these embodiments, Ring C is

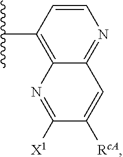

wherein R^{cA} is an independently selected R^{c}.

In certain embodiments (when Ring C is

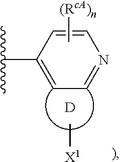

Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

As non-limiting examples of the foregoing embodiments, Ring C can be selected from the group consisting of:

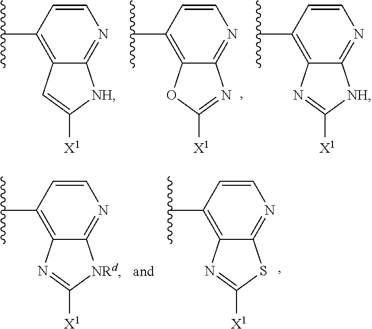

each further optionally substituted with R^{cA}, wherein each R^{cA} is an independently selected R^{c}. For example, Ring C can be

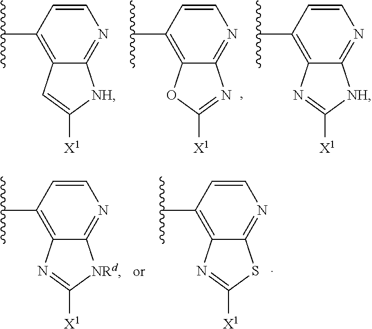

In certain embodiments, Ring C is

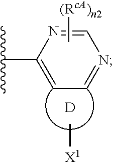

Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n2 is 0 or 1; and each R^{cA} is an independently selected R^{c}.

In certain of these embodiments, Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

As non-limiting examples of the foregoing embodiments, Ring C can be selected from the group consisting of:

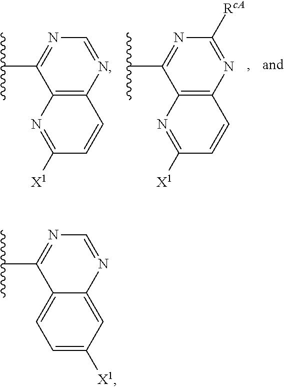

each further optionally substituted with R^{cA}, wherein each R^{cA} is an independently selected R^{c}.

In certain embodiments (when Ring C is

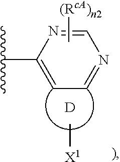

Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In some embodiments, Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{cA}, wherein each R^{cA} is an independently selected R^{c}.

In certain of these embodiments, Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

In certain of the foregoing embodiments, Ring C is selected from the group consisting of: pyrazolyl, imidazolyl, thiazolyl, oxazolyl, triazolyl, furanyl, thiophenyl, oxadiazolyl, and thiadiazolyl, each optionally substituted with from 1-2 R^{cA}, wherein a ring nitrogen atom is optionally substituted with R^{d}, and each R^{cA} is an independently selected R^{c}.

As non-limiting examples of the foregoing embodiments, Ring C can be selected from the group consisting of:

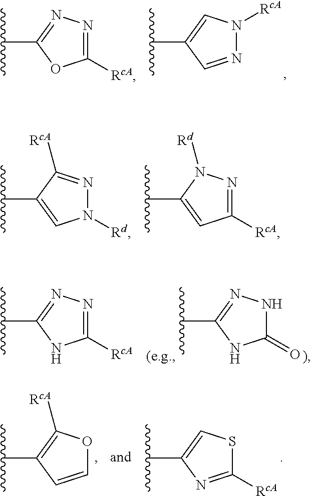

In certain embodiments, Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is substituted with X¹ and further optionally substituted with from 1-2 R^{cA}, wherein each R^{cA} is an independently selected R^{c}.

In certain embodiments, Ring C is selected from the group consisting of: pyrazolyl, imidazolyl, thiazolyl, oxazolyl, triazolyl, furanyl, thiophenyl, oxadiazolyl, and thiadiazolyl, each substituted with X¹ and further optionally substituted with from 1-2 R^{cA}, wherein a ring nitrogen atom is optionally substituted with R^{d}, and each R^{cA} is an independently selected R^{c}. For example, Ring C can be

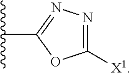

In some embodiments, Ring C is 2-pyridonyl or 4-pyridonyl, each optionally substituted with X¹ and further optionally substituted with from 1-4 R^{cA}, wherein the ring nitrogen atom is optionally substituted with R^{d}, wherein each R^{cA} is an independently selected R^{c}.

In certain of these embodiments, Ring C is 2-pyridonyl which is optionally substituted with X¹ and further optionally substituted with from 1-4 R^{cA}, wherein the ring nitrogen atom is optionally substituted with R^{d}, wherein each R^{cA} is an independently selected R^{c}.

In certain of the foregoing embodiments, Ring C is 2-pyridonyl which is optionally substituted with from 1-4 R^{cA}, wherein the ring nitrogen atom is optionally substituted with R^{d}, wherein each R^{cA} is an independently selected R^{c}. For example, Ring C can be

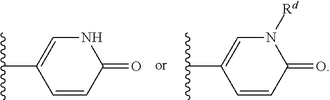

In some embodiments, Ring C is

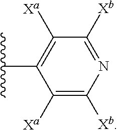

In certain of the foregoing embodiments, Ring C is

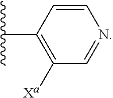

In certain of these embodiments, each X^{a} is selected from the group consisting of: H; halo; and C₁₋₆ alkyl optionally substituted with from 1-6 R^{a}.

In certain of these embodiments, from 1-2, such as 1, occurrence of X^{a} is an independently substituent other than H.

In certain of these embodiments, one occurrence of X^{a} is halo, such as —F or —Cl. For example, one occurrence of X^{a} is —F.

In certain of these embodiments, one occurrence of X^{a} is C₁₋₃ alkyl optionally substituted with from 1-6 R^{a}. For example, one occurrence of X^{a} is C₁₋₃ alkyl substituted with from 1-3 independently selected halo, such as —CF₃ or —CHF₂.

In certain of these embodiments, each X^{a} is —H.

In certain of the foregoing embodiments, wherein Ring C is

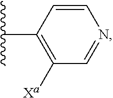

wherein X^{a} is selected from the group consisting of: —F, —Cl, —H, and C₁₋₆ alkyl optionally substituted with from 1-6 R^{a}.

In certain of the forgoing embodiments, X^{a} is —F.

In certain of the forgoing embodiments, X^{a} is —Cl.

In certain of the forgoing embodiments, X^{a} is —H.

In certain of the forgoing embodiments, X^{a} is C₁₋₃ alkyl substituted with from 1-3 independently selected halo, such as —CF₃ or —CHF₂.

In certain of the foregoing embodiments, Ring C is

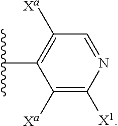

For example, Ring C can be

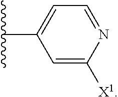

In certain embodiments, Ring C is

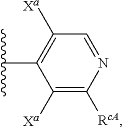

wherein R^{cA} is an independently selected R^{c}. For example, Ring C can be

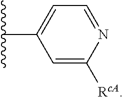

In certain of the foregoing embodiments, each X^{a} is selected from the group consisting of: H; halo; and C₁₋₆ alkyl optionally substituted with from 1-6 R^{a}.

In certain of the foregoing embodiments, 1-2, such as 1, occurrence of X^{a} is an independently substituent other than H.

In certain of the foregoing embodiments, one occurrence of X^{a} is halo, such as —F or —Cl.

In certain of the foregoing embodiments, one occurrence of X^{a} is —F

In certain of the foregoing embodiments, one occurrence of X^{a} is C₁₋₃ alkyl optionally substituted with from 1-6 R^{a}.

In certain of the foregoing embodiments, one occurrence of X^{a} is C₁₋₃ alkyl substituted with from 1-3 independently selected halo, such as but not limited to —CF₃ or —CHF₂.

In certain of the foregoing embodiments, each X^{a} is —H.

In some embodiments, Ring C is C₆₋₁₀ aryl optionally substituted with X¹ and further optionally substituted with from 1-4 R^{cA}, wherein each R^{cA} is an independently selected R^{c}.

In certain of the foregoing embodiments, Ring C is phenyl optionally substituted with from 1-4 R^{cA}, wherein each R^{cA} is an independently selected R^{c}. For example, Ring C can be

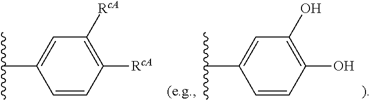

In some embodiments, Ring C is heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{cA}, wherein each R^{cA} is an independently selected R^{c}.

In certain of these embodiments, Ring C is heterocyclyl including from 4-8, such as 5-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with X¹ and further optionally substituted with from 1-4 substituents independently selected from the group consisting of oxo and R^{cA}, wherein each R^{cA} is an independently selected R^{c}. For example, Ring C can be

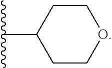 [SMILES: *C1CCOCC1]

Variables m, X², L¹, and R⁵

In certain embodiments, m is 1. In some embodiments, m is 0.

In certain embodiments, X² is selected from the group consisting of: —O—, —N(R^{N})—, and —S(O)₀₋₂. In certain of these embodiments, X² is —N(R^{N})—. For example, X² can be —N(H)—. As another non-limiting example, X² can be —O—.

In certain embodiments, X² is selected from the group consisting of: —OC(═O)—*, —N(R^{N})C(═O)—*, and —N(R^{N})S(O)₁₋₂—*. In certain of these embodiments, X² is —N(R^{N})C(═O)—*. For example, X² can be —N(H)C(═O)—*. In certain embodiments, X² is —N(R^{N})S(O)₂—*. For example, X² can be —NHS(O)₂—.

In certain embodiments, X² is selected from the group consisting of: —OC(═O)N(R^{N})—*, —N(R^{N})C(═O)O—*, —N(R^{N})C(═O)N(R^{N})—*, and —N(R^{N})S(O)₁₋₂N(R^{N})—*. In certain of these embodiments, X² is —N(R^{N})C(═O)O—*. For example, X² can be —N(H)C(═O)O—*. X² is —N(R^{N})C(═O)N(R^{N})—*, such as —N(H)C(═O)N(H)—*.

In certain embodiments, X² is —C(═O)O—*, —C(═O)N(R^{N})—*, or —S(O)₁₋₂N(R^{N})—*. In certain of these embodiments, X² is —C(═O)N(R^{N})—*. For example, X² can be —C(═O)N(H)—*.

In certain embodiments, X² is

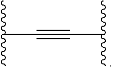 [SMILES: *C#C*]

In certain embodiments, X² is C₂₋₆ alkenylene optionally substituted with from 1-3 R^{a}. For example, X² can be

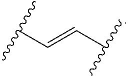 [SMILES: */C=C/*]

In certain embodiments, L¹ is a bond.

In certain embodiments, L¹ is C₁₋₁₀ alkylene optionally substituted with from 1-6 R^{a}.

In certain of these embodiments, L¹ is C₁₋₃ alkylene optionally substituted with from 1-6 R^{a}. In certain of the foregoing embodiments, L¹ is unsubstituted C₁₋₃ alkylene. As non-limiting examples of the foregoing embodiments, L¹ can be —CH₂—, —CH₂CH₂—, —CH₂CF₂—, or —CH(Me)-. For example, L¹ can be —CH₂—, —CH₂CH₂—, or —CH(Me)-.

In certain embodiments, L¹ is branched C₃₋₆ alkylene optionally substituted with from 1-6 R^{a}. For example, L¹ can be

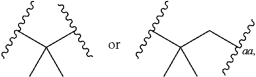

wherein aa is the point of attachment to R⁵.

In certain embodiments, R⁵ is —C₁₋₆ alkoxy or —S(O)₀₋₂(C₁₋₆ alkyl), each optionally substituted with from 1-6 R^{a}. In certain of these embodiments, R⁵ is —C₁₋₆ alkoxy optionally substituted with from 1-6 R^{a}. As a non-limiting example of the foregoing embodiments, R⁵ can be —C₁₋₃ alkoxy. For example, R⁵ can be methoxy.

In certain embodiments, R⁵ is H or halo. As non-limiting examples of the foregoing embodiments, R⁵ can be H or —F. For example, R⁵ can be H.

In certain embodiments, R⁵ is —OH or —NR^{e}R^{f}. For example, R⁵ can be —OH.

In certain embodiments, R⁵ is —R^{g}.

In certain of these embodiments, R⁵ is selected from the group consisting of:
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c}.

In certain of the foregoing embodiments, R⁵ is C₆₋₁₀ aryl optionally substituted with from 1-4 R^{c}. In certain of these embodiments, R⁵ is phenyl optionally substituted with from 1-4 R^{c}. As non-limiting examples of the foregoing embodiments, R⁵ can be phenyl optionally substituted with from 1-2 independently selected halo, such as —F.

In certain embodiments, R⁵ is heteroaryl including from 5-10 ring atoms, wherein from 1-4 ring atoms are heteroatoms, each independently selected from the group consisting of N, N(H), N(R^{d}), 0, and S(O)₀₋₂, and wherein the heteroaryl is optionally substituted with from 1-4 R^{c}.

In certain of the foregoing embodiments, R⁵ is heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

In certain of these embodiments, R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}. For example, R⁵ can be

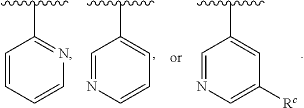

In certain embodiments, R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}. For example, R⁵ can be

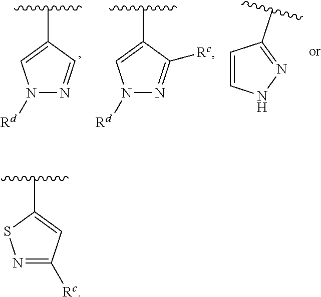

In certain embodiments, R⁵ is selected from the group consisting of:
C₃₋₁₀ cycloalkyl or C₃₋₁₀ cycloalkenyl, each of which is optionally substituted with from 1-4 substituents independently selected from the group consisting of oxo and R^{c}; and
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

In certain of these embodiments, R⁵ is C₃₋₁₀ cycloalkyl or C₃₋₁₀ cycloalkenyl, each of which is optionally substituted with from 1-4 substituents independently selected from the group consisting of oxo and R^{c}.

In certain of the foregoing embodiments, R⁵ is C₃₋₁₀ cycloalkyl (e.g., C₃₋₆ cycloalkyl) optionally substituted with from 1-4 R^{c}, such as wherein R⁵ is cyclopropyl.

In certain embodiments, R⁵ is heterocyclyl or heterocycloalkenyl including from 3-ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

In certain of these embodiments, R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R⁵ can be

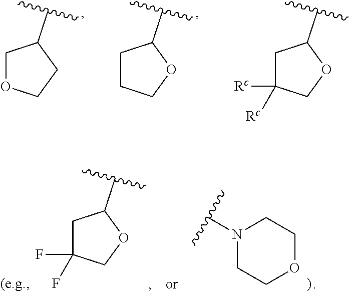

In certain embodiments, R⁵ is selected from the group consisting of: —R^{g2}-R^{W} and —R^{g2}-R^{Y}. In certain of these embodiments, R⁵ is —R^{g2}-R^{Y}.

In certain embodiments, the —R^{g2} group present in R⁵ is C₆₋₁₀ arylene optionally substituted with from 1-4 R^{c}.

In certain of these embodiments, the —R^{g2} group present in R⁵ is phenylene optionally substituted with from 1-4 R^{c}.

In certain of the foregoing embodiments, the —R^{g2} group present in R⁵ is 1,3-phenylene or 1,4-phenylene, each optionally substituted with from 1-4 R^{c}. As non-limiting examples of the foregoing embodiments, —R^{g2} can be

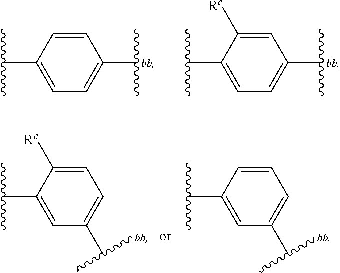

wherein bb is the point of attachment to R^{Y}.

In certain embodiments, the R^{Y} group present in R⁵ is —R^{g}.

In certain of these embodiments, the R^{Y} group present in R⁵ is heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

In certain of the foregoing embodiments, the R^{Y} group present in R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as wherein R^{Y} is

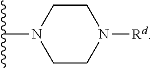

In certain embodiments, R⁵ is -L⁵-R^{g}.

In certain of these embodiments, R⁵ is —O—R^{g}.

In certain embodiments, R⁵ is —O—(C₆₋₁₀ aryl) wherein the C₆₋₁₀ aryl is optionally substituted with from 1-4 R^{c}.

As a non-limiting example of the foregoing embodiments, R⁵ can be —O-phenyl wherein the phenyl is optionally substituted with from 1-2 R^{c}. For example, R⁵ can be

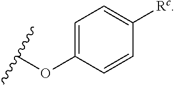

Non-Limiting Combinations of m, X², L¹, and R⁵

[AA]:

In certain embodiments, X¹ is —(X²)ₘ-L¹-R⁵, wherein:
m is 0 or 1;
X² is —N(R^{N})— or —O—;
L¹ is a bond or C₁₋₆ alkylene optionally substituted with from 1-3 R^{a}; and
R⁵ is —R^{g}.

In certain embodiments of [AA], R⁵ is phenyl optionally substituted with from 1-4 R^{c}, such as wherein R⁵ is phenyl optionally substituted with from 1-2 independently selected halo, such as —F.

In certain embodiments of [AA], R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

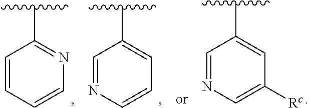

In certain embodiments of [AA], R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

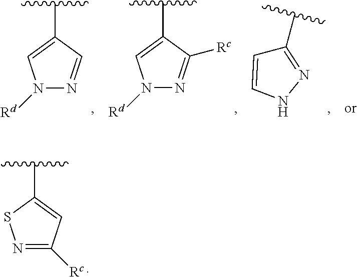

In certain embodiments of [AA], R⁵ is C₃₋₁₀ cycloalkyl, such as C₃₋₆ cycloalkyl, optionally substituted with from 1-4 R^{c}, such as wherein R⁵ is cyclopropyl.

In certain embodiments of [AA], R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R⁵ can be

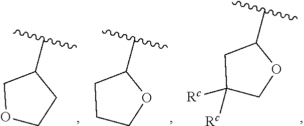

such as

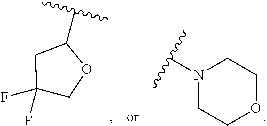 [SMILES: *C1CC(F)(F)CO1 ; *N1CCOCC1]

In certain embodiments of [AA], m is 0.

In certain embodiments of [AA], m is 1.

In certain embodiments of [AA], X² is —N(R^{N})— (e.g., N(H)).

In certain embodiments of [AA], X² is —O—.

In certain embodiments of [AA], L¹ is a bond.

In certain embodiments of [AA], L¹ is C₁₋₃ alkylene (e.g., —CH₂—, —CH₂CH₂—, or —CH(Me)-).

In certain embodiments of [AA], L¹ is branched C₃₋₆ alkylene. For example, L¹ can be

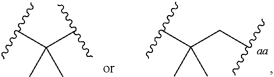

wherein aa is the point of attachment to R⁵.

[BB]:

In certain embodiments, X¹ is —X²-L¹-R⁵, wherein:
X² is —N(R^{N})C(═O)—*, —N(R^{N})S(O)₂—*, —N(R^{N})C(═O)O—*, or N(R^{N})C(═O)N(R^{N})*;
L¹ is a bond or C₁₋₆ alkylene optionally substituted with from 1-3 R^{a}; and
R⁵ is —R^{g}.

In certain embodiments of [BB], R⁵ is phenyl optionally substituted with from 1-4 R^{c}, such as wherein R⁵ is phenyl optionally substituted with from 1-2 independently selected halo, such as —F.

In certain embodiments of [BB], R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

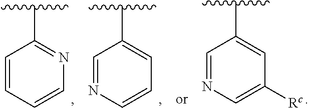

In certain embodiments of [BB], R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

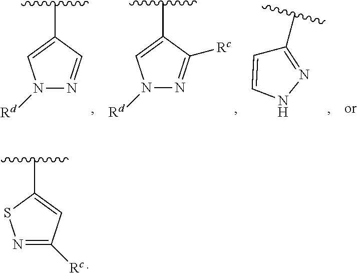

In certain embodiments of [BB], R⁵ is C₃₋₁₀ cycloalkyl, such as C₃₋₆ cycloalkyl, optionally substituted with from 1-4 R^{c}, such as wherein R⁵ is cyclopropyl.

In certain embodiments of [BB], R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R⁵ can be

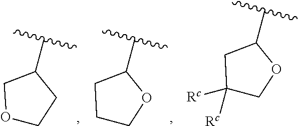

such as

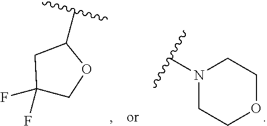 [SMILES: *C1CC(F)(F)CO1 ; *N1CCOCC1]

In certain embodiments of [BB], X² is —N(R^{N})C(═O)—* (e.g., —N(H)C(═O)—*).

In certain embodiments of [BB], X² is —N(R^{N})S(O)₂—, such as —N(H)S(O)₂—*.

In certain embodiments of [BB], X² is —N(R^{N})C(═O)O—*, or —N(R^{N})C(═O)N(R^{N})—* (e.g., —N(H)C(═O)O—*; e.g., —N(H)C(═O)N(H)—*).

In certain embodiments of [BB], L¹ is a bond.

In certain embodiments of [BB], L¹ is C₁₋₃ alkylene (e.g., —CH₂—, —CH₂CH₂—, or —CH(Me)-).

In certain embodiments of [BB], L¹ is branched C₃₋₆ alkylene. For example, L¹ can be

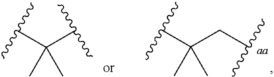

wherein aa is the point of attachment to R⁵.

[CC]:

In certain embodiments, X¹ is —X²-L¹-R⁵, wherein:
X² is

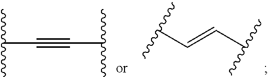

L¹ is a bond or C₁₋₆ alkylene optionally substituted with from 1-3 R^{a}; and
R⁵ is —R^{g}.

In certain embodiments of [CC], R⁵ is phenyl optionally substituted with from 1-4 R^{c}, such as wherein R⁵ is phenyl optionally substituted with from 1-2 independently selected halo, such as —F.

In certain embodiments of [CC], R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

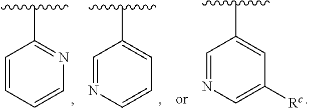

In certain embodiments of [CC], R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

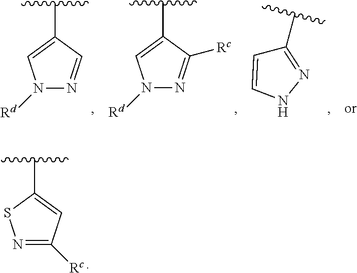

In certain embodiments of [CC], R⁵ is C₃₋₁₀ cycloalkyl, such as C₃₋₆ cycloalkyl, optionally substituted with from 1-4 R^{c}, such as wherein R⁵ is cyclopropyl.

In certain embodiments of [CC], R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R⁵ can be

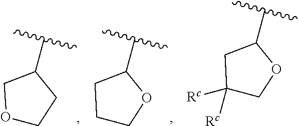

such as

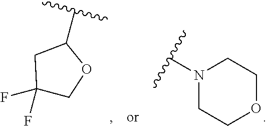 [SMILES: *C1CC(F)(F)CO1 ; *N1CCOCC1]

In some embodiments of [CC], X² is

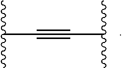 [SMILES: *C#C*]

In some embodiments of [CC], X² is

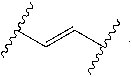 [SMILES: */C=C/*]

In certain embodiments of [CC], L¹ is a bond.

In certain embodiments of [CC], L¹ is C₁₋₃ alkylene (e.g., —CH₂—, —CH₂CH₂—, or —CH(Me)-).

In certain embodiments of [CC], L¹ is branched C₃₋₆ alkylene. For example, L¹ can be

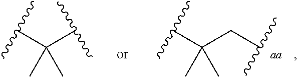

wherein aa is the point of attachment to R⁵.

[DD]:

In certain embodiments, X¹ is —(X²)ₘ-L¹-R⁵, wherein:
m is 0 or 1;
X² is —N(R^{N})— or —O—;
L¹ is a bond or C₁₋₆ alkylene optionally substituted with from 1-3 R^{a}; and
R⁵ is —R^{g2}-R^{Y}.

In certain embodiments of [DD], the —R^{g2} group present in R⁵ is 1,3-phenylene or 1,4-phenylene, each optionally substituted with from 1-4 R^{c}, such as wherein —R^{g2} is

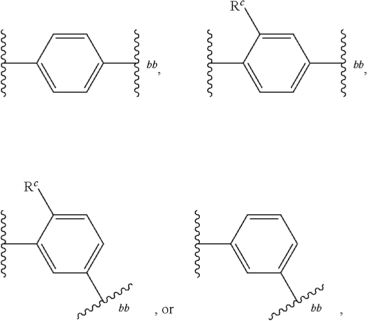

wherein bb is the point of attachment to R^{Y}.

In certain embodiments of [DD], the R^{Y} group present in R⁵ is —R^{g}.

In certain embodiments of [DD], the R^{Y} group present in R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R^{Y} can be

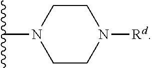

In certain embodiments of [DD], X² is —N(R^{N})— (e.g., N(H)).

In certain embodiments of [DD], X² is —O—.

In certain embodiments of [DD], L¹ is a bond.

In certain embodiments of [DD], L¹ is C₁₋₃ alkylene (e.g., —CH₂—, —CH₂CH₂—, or —CH(Me)-).

In certain embodiments of [DD], L¹ is branched C₃₋₆ alkylene. For example, L¹ can be

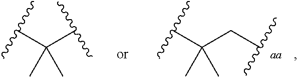

wherein aa is the point of attachment to R⁵.

[EE]:

In certain embodiments, X¹ is —X²-L¹-R⁵, wherein:
X² is —N(R^{N})—, —O—, —N(R^{N})C(═O)—*, —N(R^{N})S(O)₂—, —N(R^{N})C(═O)O—*, or —N(R^{N})C(═O)N(R^{N})—*.
L¹ is C₁₋₆ alkylene optionally substituted with from 1-3 R^{a}; and
R⁵ is H, halo, C₁₋₆ alkoxy optionally substituted with from 1-3 R^{a}, or —OH.

In certain embodiments of [EE], R⁵ is H.

In certain embodiments of [EE], R⁵ is halo (e.g., —F).

In certain embodiments of [EE], R⁵ is C₁₋₆ alkoxy optionally substituted with from 1-3 R^{a}, such as wherein R⁵ is C₁₋₃ alkoxy such as methoxy.

In certain embodiments of [EE], R⁵ is —OH.

In certain embodiments of [EE], X² is —N(R^{N})— (e.g., N(H)).

In certain embodiments of [EE], X² is —O—.

In certain embodiments of [EE], X² is —N(R^{N})C(═O)—* (e.g., —N(H)C(═O)—*).

In certain embodiments of [EE], X² is —N(R^{N})S(O)₂—, such as —N(H)S(O)₂—*.

In certain embodiments of [EE], X² is —N(R^{N})C(═O)O—*, or —N(R^{N})C(═O)N(R^{N})—* (e.g., —N(H)C(═O)O—*; e.g., —N(H)C(═O)N(H)—*).

In certain embodiments of [EE], L¹ is C₁₋₃ alkylene (e.g., —CH₂—, —CH₂CH₂—, or —CH(Me)-).

In certain embodiments of [EE], L¹ is branched C₃₋₆ alkylene. For example, L¹ can be

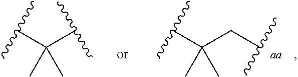

wherein aa is the point of attachment to R⁵.

[FF]:

In certain embodiments, X¹ is -L¹-R⁵, wherein L¹ is C₁₋₆ alkylene optionally substituted with from 1-3 R^{a}; and R⁵ is -L⁵-R^{g}.

In certain embodiments of [FF], R⁵ is —O—R^{g}.

In certain embodiments of [FF], R⁵ is —O-(phenyl), wherein the phenyl is optionally substituted with from 1-2 R^{c}.

In certain embodiments of [FF], L¹ is C₁₋₃ alkylene (e.g., —CH₂—, —CH₂CH₂—, or —CH(Me)-).

Variable R^{cA}

In certain embodiments, each occurrence of R^{cA} is independently selected from the group consisting of: halo; cyano; C₁₋₁₀ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —NR^{e}R^{f}; —OH; —S(O)₁₋₂NR′R″; —C₁₋₄ thioalkoxy; —C(═O)(C₁₋₁₀ alkyl); —C(═O)O(C₁₋₄ alkyl); —C(═O)OH; and —C(═O)NR′R″.

In certain embodiments, one occurrence of R^{cA} is —NR^{e}R^{f}.

In certain of these embodiments, one occurrence of R^{cA} is —NH₂.

In certain of the foregoing embodiments, one occurrence of R^{cA} is —NH(C₁₋₆ alkyl), wherein the C₁₋₆ alkyl is optionally substituted with from 1-3 substituents each independently selected from the group consisting of NR′R″, —OH, C₁₋₆ alkoxy, C₁₋₆ haloalkoxy, and halo. For example, one occurrence of R^{cA} can be —NHMe, —NHCH₂CF₃, —NHCH₂CH₂OH, or -NHiPr.

In certain embodiments, one occurrence of R^{cA} is —NHC(═O)C₁₋₄ alkyl, such as NHC(═O)CH₃.

In certain embodiments, one occurrence of R^{cA} is N(C₁₋₃ alkyl)₂ such as NMe₂.

In certain embodiments, one occurrence of R^{cA} is C₁₋₄ alkoxy optionally substituted with C₁₋₄ alkoxy or C₁₋₄ haloalkoxy. For example, R^{cA} can be OMe or OCH₂CH₂OMe.

In certain embodiments, one occurrence of R^{cA} is C₁₋₄ haloalkoxy (e.g., —OCH₂CF₃).

In certain embodiments, one occurrence of R^{cA} is C₁₋₄ thioalkoxy (e.g., —SCH₃).

In certain embodiments, one occurrence of R^{cA} is C₁₋₆ alkyl, such as methyl; or wherein one occurrence of R^{cA} is C₁₋₃ alkyl substituted with from 1-6 independently selected halo. For example, one occurrence of R^{cA} can be —CF₃.

In certain embodiments, one occurrence of R^{cA} is C₁₋₆ alkyl substituted with R^{a}, such as C₁₋₆ alkyl substituted with C₁₋₃ alkoxy or C(═O)NR′R″. For example, one occurrence of R^{cA} can be

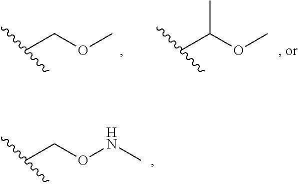

In certain embodiments, one occurrence of R^{cA} is halo (e.g., —F).

In certain embodiments, one occurrence of R^{cA} is —OH.

In certain embodiments, one occurrence of R^{cA} is C(═O)NR′R″ (e.g., C(═O)NHMe).

Variables R^{1c}, R^{2a}, R^{2b}, R^{3a}, and R^{3b}

In some embodiments, R^{1c} is H.

In some embodiments, R^{2a} and R^{2b} are both H.

In some embodiments, from 1-2 (e.g., 1 or 2) of R^{2a} and R^{2b} is an independently selected substituent that is other than H.

In certain of these embodiments, one of R^{2a} and R^{2b} (e.g., R^{2a}), is a substituent that is other than H.

In certain of the foregoing embodiments, one of R^{2a} and R^{2b} (e.g., R^{2a}), is R^{b}. In certain of these embodiments, one of R^{2a} and R^{2b} (e.g., R^{2a}) is C₁₋₆ alkyl which is optionally substituted with from 1-6 R^{a}. In certain of these embodiments, one of R^{2a} and R^{2b} (e.g., R^{2a}) is C₁₋₃ alkyl, such as methyl or ethyl. In certain embodiments (when one of R^{2a} and R^{2b} is as defined supra), the other of R^{2a} and R^{2b} (e.g., R^{2b}) is H.

In some embodiments, R^{3a} and R^{3b} are both H.

In some embodiments, from 1-2 (e.g., 1 or 2) of R^{3a} and R^{3b} is an independently selected substituent that is other than H.

In certain of the foregoing embodiments, one of R^{3a} and R^{3b} (e.g., R^{3a}) is a substituent that is other than H. In certain of these embodiments, one of R^{3a} and R^{3b} (e.g., R^{3a}) is R^{b}. In certain of these embodiments, one of R^{3a} and R^{3b} (e.g., R^{3a}) is C₁₋₆ alkyl which is optionally substituted with from 1-6 R^{a}. For example, one of R^{3a} and R^{3b} (e.g., R^{3a}) can be C₁₋₃ alkyl, such as methyl or ethyl. In certain embodiments (when one of R^{3a} and R^{3b} is as defined supra), the other of R^{3a} and R^{3b} (e.g., R^{3b}) is H.

In some embodiments, one of R^{3a} and R^{3b}, such as R^{3a}, is C₁₋₃ alkyl optionally substituted with from 1-3 independently selected halo. As non-limiting examples of the foregoing embodiments, one of R^{3a} and R^{3b}, such as R^{3a}, is —CH₃, —CH₂CH₃, —CH₂F, —CHF₂, —CF₃, —CH₂CHF₂, or —CH₂CH₂F.

In some embodiments, one of R^{3a} and R^{3b}, such as R^{3a}, is C₁₋₃ alkyl substituted with C₁₋₄ alkoxy, C₁₋₄ haloalkoxy, or NR^{e}R^{f}. As non-limiting examples of the foregoing embodiments, one of R^{3a} and R^{3b}, such as R^{3a}, is —CH₂OMe, —CH₂CH₂OMe, —CH(Me)CH₂OMe, —CH₂CH(Me)OMe, -CH₂OEt, —CH₂CH₂OCHF₂, —CH₂NR^{e}R^{f} (e.g., —CH₂N(CF₃)Me), or —CH₂CH₂NR^{e}R^{f} (e.g., —CH₂CH₂NMe₂).

In some embodiments, one of R^{3a} and R^{3b}, such as R^{3a}, is C₁₋₃ alkyl substituted with C₁₋₄ alkoxy. As non-limiting examples of the foregoing embodiments, one of R^{3a} and R^{3b}, such as R^{3a}, is —CH₂OMe, —CH₂CH₂OMe, —CH(Me)CH₂OMe, —CH₂CH(Me)OMe, or —CH₂OEt, such as —CH₂OMe.

In some embodiments, one of R^{3a} and R^{3b}, such as R^{3a}, is C₁₋₃ alkyl substituted with C₁₋₄ alkoxy. As non-limiting examples of the foregoing embodiments, one of R^{3a} and R^{3b}, such as R^{3a}, is —CH₂OMe, —CH₂CH₂OMe, —CH(Me)CH₂OMe, —CH₂CH(Me)OMe, or —CH₂OEt, such as —CH₂OMe; such as —CH₂CH₂OMe; optionally the other one of R^{3a} and R^{3b}, such as R^{3b} is H.

In some embodiments, one of R^{3a} and R^{3b}, such as R^{3a}, is C₁₋₃ alkyl substituted with C₁₋₄ alkoxy, C₁₋₄ haloalkoxy, or NR^{e}R^{f} and further substituted with from 1-3 independently selected halo. In certain embodiments, one of R^{3a} and R^{3b}, such as R^{3a}, is C₁₋₃ alkyl substituted with C₁₋₄ alkoxy and further substituted with from 1-3 independently selected halo. For example, one of R^{3a} and R^{3b}, such as R^{3a}, can be

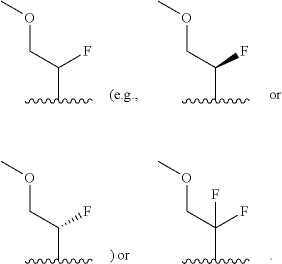

In some embodiments, one of R^{3a} and R^{3b}, such as R^{3a}, is C₃₋₆ alkyl substituted with C₁₋₄ alkoxy, C₁₋₄ haloalkoxy, or NR^{e}R^{f}. In certain of these embodiments, one of R^{3a} and R^{3b}, such as R^{3a}, is branched C₃₋₆ alkyl substituted with C₁₋₄ alkoxy, C₁₋₄ haloalkoxy, or NR^{e}R^{f}. In certain of the foregoing embodiments, one of R^{3a} and R^{3b}, such as R^{3a}, is branched C₃₋₆ alkyl substituted with C₁₋₄ alkoxy. For example, one of R^{3a} and R^{3b}, such as R^{3a}, can be

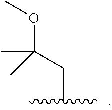 [SMILES: *CC(C)(C)OC]

In some embodiments, one of R^{3a} and R^{3b}, such as R^{3a}, is R^{g} or -(L^{g})_{g}-R^{g}.

In certain of the foregoing embodiments, one of R^{3a} and R^{3b}, such as R^{3a}, is selected from the group consisting of:
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
C₃₋₆ cycloalkyl optionally substituted with from 1-4 R^{c}.

As non-limiting examples of the foregoing embodiments, one of R^{3a} and R^{3b}, such as R^{3a}, is selected from the group consisting of: cyclopropyl, cyclobutyl, oxetanyl, and azetidinyl, each of which is optionally substituted with from 1-2 substituents independently selected from the group consisting of: C₁₋₃ alkyl and halo, wherein the ring nitrogen of the azetidinyl is optionally substituted with R^{d}.

In certain of the foregoing embodiments, one of R^{3a} and R^{3b}, such as R^{3a}, is —(C₁₋₃ alkylene)-R^{g} or —(C₁₋₃ alkylene)-O—R^{g}, and optionally the R^{g} group of R^{3a} or R^{3b} is: C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

In certain of the foregoing embodiments, one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is selected from the group:
C₃₋₆ cycloalkyl (e.g., cyclopropyl, cyclobutyl) optionally substituted with from 1-4 R^{c}, or
heterocyclyl including from 4-6 ring atoms (e.g., oxetanyl, azetidinyl), wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c} (e.g., C₁₋₃ alkyl, halo).

In certain of the foregoing embodiments, one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is selected from the group consisting of:
cyclopropyl, cyclobutyl, oxetanyl, and azetidinyl, each of which is optionally substituted with from 1-2 substituents independently selected from the group consisting of: C₁₋₃ alkyl and halo, wherein the ring nitrogen of the azetidinyl is optionally substituted with R^{d}.

In certain of the foregoing embodiments, one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is selected from the group consisting of:
cyclopropyl, cyclobutyl, oxetanyl, 1,4-dioxanyl, and azetidinyl, each of which is optionally substituted with from 1-2 substituents independently selected from the group consisting of: C₁₋₃ alkyl and halo, wherein the ring nitrogen of the azetidinyl is optionally substituted with R^{d}.

As non-limiting examples of the foregoing embodiments, one of R^{3a} and R^{3b}, such as R^{3a}, can be selected from the group consisting of:

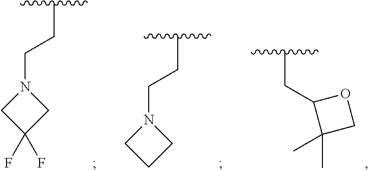

such as

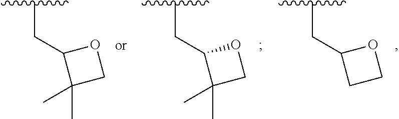

such as

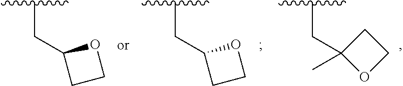

such as

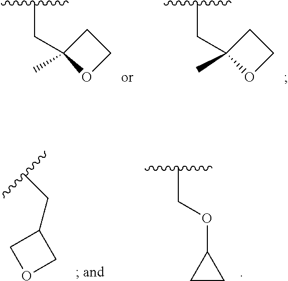

As further non-limiting examples of the forgoing embodiments, one of R^{3a} and R^{3b}, such as R^{3a}, can be selected from the group consisting of:

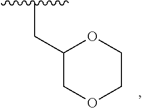 [SMILES: *CC1COCCO1]

such as

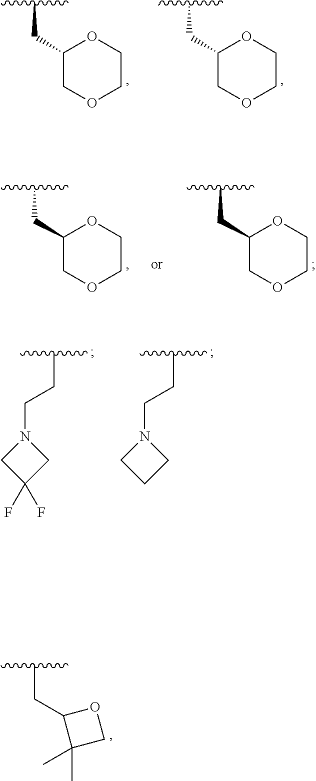

such as

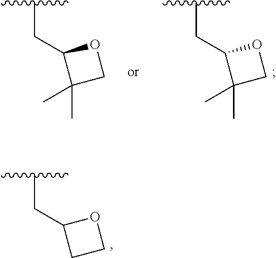

such as

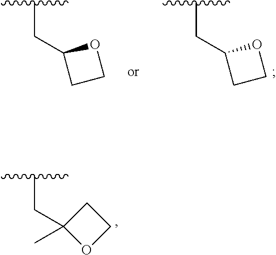

such as

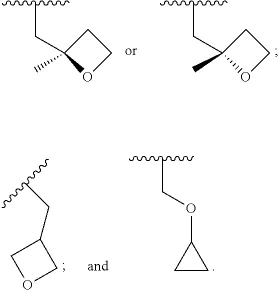

In some embodiments, one of R^{3a} and R^{3b}, such as R^{3a}, is -(L^{g})_{g}-R^{W}.

In certain embodiments, one of R^{3a} and R^{3b}, such as R^{3a}, is —(C₁₋₃ alkylene)-R^{W}; optionally one of R^{3a} and R^{3b}, such as R^{3a}, is —CH₂—R^{W}, or —CH₂CH₂—R^{W}.

In certain embodiments, the R^{W} group of R^{3a} or R^{3b} is: C(═O)—CH═CH₂, or —NHC(═O)—CH═CH₂.

As a non-limiting example, one of R^{3a} and R^{3b}, such as R^{3a}, can be

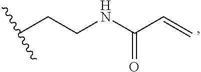 [SMILES: *CCNC(=O)C=C]

such as

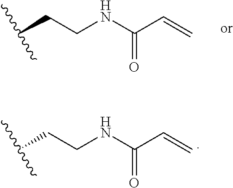

In some embodiments, one of R^{3a} and R^{3b}, such as R^{3a}, is -(L^{g})_{g}-R^{g2}-R^{W}.

In some embodiments, one of R^{3a} and R^{3b}, such as R^{3a}, is —(C₁₋₃ alkylene)-R^{g2}-R^{W}, and optionally one of R^{3a} and R^{3b}, such as R^{3a}, is —CH₂—R^{g2}-R^{W}, or —CH₂CH₂—R^{g2}-R^{W}.

In certain of these embodiments, the RO group of R^{3a} or R^{3b} is

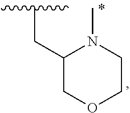 [SMILES: *CC1COCCN1C]

such as

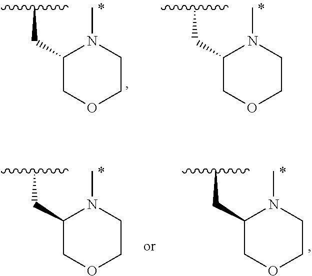

or wherein the waveline represents the point of attachment to L^{g} (e.g., —CH₂— or —CH₂CH₂—) and the asterisk represents the point of attachment to R^{W}; and wherein the R^{W} group of R^{3a} or R^{3b} is C(═O)—CH═CH₂, or —NHC(═O)—CH═CH₂.

In certain of these embodiments, one of R^{3a} and R^{3b}, such as R^{3a}, is —CH₂—R^{g2}-R^{W}, and wherein the R^{g2} group of R^{3a} or R^{3b} is

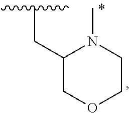 [SMILES: *CC1COCCN1C]

such as

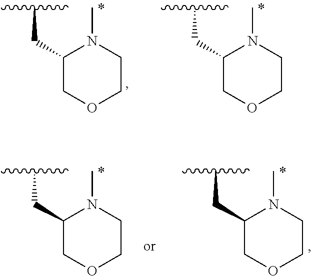

wherein the waveline represents the point of attachment to L^{g} (e.g., —CH₂— or —CH₂CH₂—) and the asterisk represents the point of attachment to R^{W}; and wherein the R^{W} group of R^{3a} or R^{3b} is C(═O)—CH═CH₂, or —NHC(═O)—CH═CH₂.

As a non-limiting example, one of R^{3a} and R^{3b}, such as R^{3a}, is

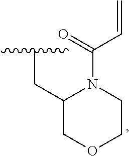 [SMILES: *CC1COCCN1C(=O)C=C]

such as

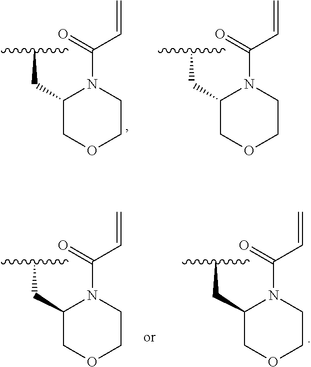

In some embodiments, the other of R^{3a} and R^{3b} is —H.

In some embodiments, the other of R^{3a} and R^{3b} is C₁₋₃ alkyl, such as methyl.

In some embodiments, the other of R^{3a} and R^{3b} is halo, such as —F.

In certain embodiments (when one of R^{3a} and R^{3b} is as defined anywhere supra), the other of R^{3a} and R^{3b} is selected from the group consisting of: —H; C₁₋₃ alkyl (e.g., methyl); and —F.

In some embodiments, R^{3a} and R^{3b}, together with the Ring B ring atom to which each is attached, form a fused saturated or unsaturated ring of 3-12 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W}.

In certain of these embodiments, R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

In certain of these embodiments, R^{3a} and R^{b}, together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

As non-limiting examples of the foregoing embodiments, R^{3a} and R^{3b}, together with the Ring B ring atom to which each is attached, form

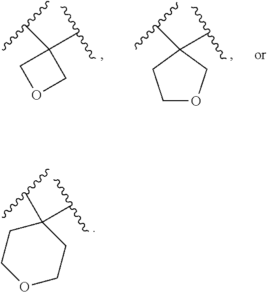 [SMILES: *C1(*)COC1 ; *C1(*)CCOC1 ; *C1(*)CCOCC1]

In certain embodiments, R^{3a} and R^{3b}, together with the Ring B ring atom to which each is attached, form:

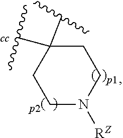

which is optionally substituted with from 1-2 substituents independently selected from the group consisting of oxo and R^{c}, wherein;
p1 and p2 are independently 0, 1, or 2;
R^{Z} is H, R^{d}, C(═O)—W, or S(O)₂W; and
cc represents the point of attachment to C(R^{2a}R^{2b}).

In certain of these embodiments, R^{3a} and R^{3b}, together with the Ring B ring atom to which each is attached, form

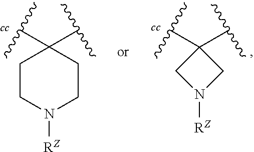

wherein R^{Z} is H, R^{d}, C(═O)—W, or S(O)₂W; and cc represents the point of attachment to C(R^{2a}R^{2b}).

In certain embodiments, R^{3a} and R^{3b}, together with the Ring B ring atom to which each is attached, form a fused ring selected from the group consisting of:

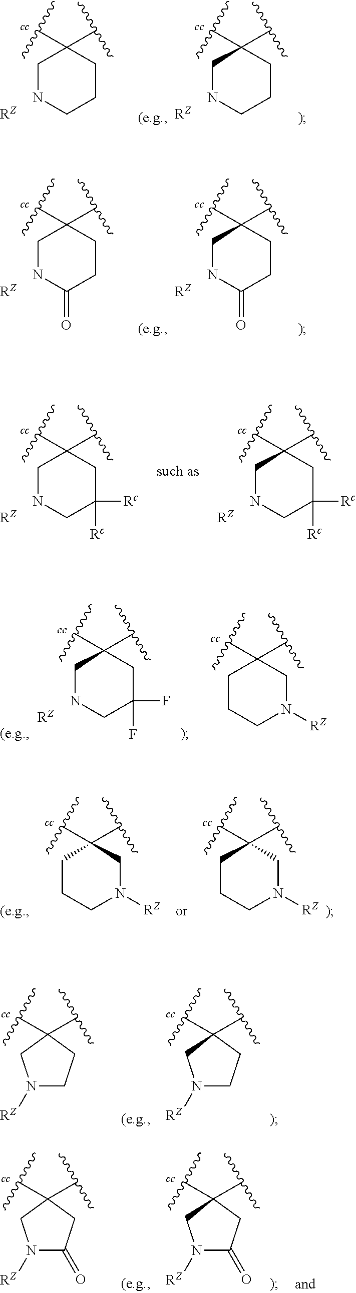

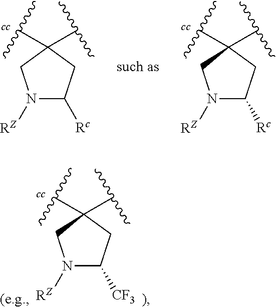

wherein R^{Z} is H, R^{d}, C(═O)—W, or S(O)₂W: and cc represents the point of attachment to C(R^{2a}R^{2b}).

In certain embodiments, R^{Z} is H.

In certain embodiments, R^{Z} is R^{d}. In certain of these embodiments, R^{Z} is C₁₋₆ alkyl optionally substituted with from 1-3 independently selected R^{a}.

In certain embodiments, R^{Z} is C(═O)—W or S(O)₂W. In certain embodiments, W is C₂₋₄ alkenyl. As a non-limiting example of the foregoing embodiments, R^{Z} can be C(═O)—CH₂═CH₂.

In certain embodiments, R^{3a} and R^{3b}, together with the Ring B ring atom to which each is attached, form a fused C₃₋₆ cycloalkyl, wherein the fused C₃₋₆ cycloalkyl is optionally substituted with from 1-2 R^{c}.

As non-limiting examples of the foregoing embodiments, R^{3a} and R^{3b}, together with the Ring B ring atom to which each is attached, form

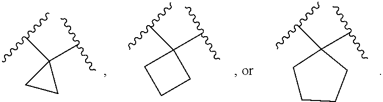 [SMILES: *C1(*)CC1 ; *C1(*)CCC1 ; *C1(*)CCCC1]

In certain of these foregoing embodiments, R^{1c}, R^{2a}, and R^{2b} are each H: and R^{3a} and R^{3b} taken together with the Ring B ring carbon atom to which each is attached form a fused C₃₋₆ (such as C₃ or C₄) cycloalkyl, wherein the fused cycloalkyl ring is optionally substituted with from 1-2 R^{c}.

In certain embodiments, one of R^{2a} and R^{2b}, such as R^{2a}, and one of R^{3a} and R^{3b}, such as R^{3a}, taken together with the Ring B ring atoms to which each is attached, form a fused saturated or unsaturated ring of 3-12 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo and R^{c}.

In certain the foregoing embodiments, one of R^{2a} and R^{2b} (such as R^{2a}) and one of R^{3a} and R^{fb} (such as R^{3a}) taken together with the Ring B ring atoms to which each is attached, form a fused saturated ring of 3-8 ring atoms:
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 3-8 ring atoms is optionally substituted with from 1-4 substituents independently selected from the group consisting of oxo and R^{c}.

In certain of these foregoing embodiments, one of R^{2a} and R^{2b}, such as R^{2a}, and one of R^{3a} and R^{3b}, such as R^{3a}, taken together with the Ring B ring atoms to which each is attached, form a fused C₃₋₆ cycloalkyl which is optionally substituted with from 1-2 R^{c}.

As non-limiting examples of the foregoing embodiments, one of R^{2a} and R^{2b} (such as R^{2a}) and one of R^{3a} and R^{3b} (such as R^{3a}) taken together with the Ring B ring atoms to which each is attached, form a fused cyclobutyl or cyclopropyl ring, e.g.,

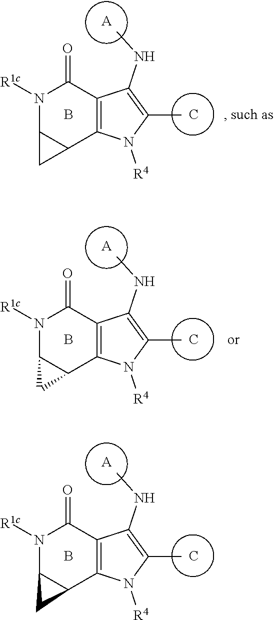

In some embodiments, one of R^{2a} and R^{2b} (such as R^{2a}) and one of R^{3a} and R^{3b} (such as R^{3a}) combine to form a double bond between the Ring B atoms to which each is attached.

In certain embodiments, the other one of R^{3a} and R^{3b} is R^{g} or -(L^{g})_{g}-R^{g}.

In certain embodiments, the other one of R^{3a} and R^{3b} is -(L^{g})_{g}-R^{g}.

In certain embodiment, the other one of R^{3a} and R^{3b} is —(C₁₋₃ alkylene)-R^{g} or —(C₁₋₃ alkylene)-O—R^{g}, and optionally the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

In certain embodiments, the other one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

In certain embodiments, the other one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is selected from the group consisting of
cyclopropyl, cyclobutyl, oxetanyl, 1,4-dioxanyl, and azetidinyl, each of which is optionally substituted with from 1-2 substituents independently selected from the group consisting of: C₁₋₃ alkyl and halo, wherein the ring nitrogen of the azetidinyl is optionally substituted with R^{d}.

In certain embodiments, the other one of R^{3a} and R^{3b}, such as R^{3a}, is selected from the group consisting of:

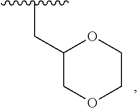 [SMILES: *CC1COCCO1]

such as

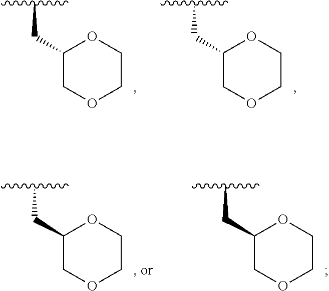

such as

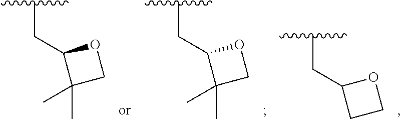

such as

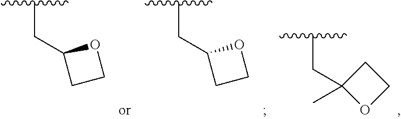

such as

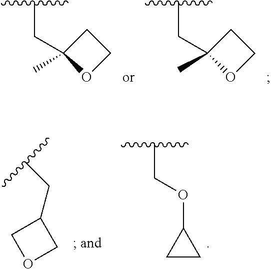

In certain embodiments, R^{1c}, R^{2a}, and R^{2b} are each H, and R^{3a} and R^{3b} are independently selected C₁₋₃ alkyl.

In certain embodiments, R^{1c}, R^{2a}, and R^{2b} are each H; one of R^{3a} and R^{3b}, such as R^{3a}, is C₁₋₃ alkyl optionally substituted with from 1-3 R^{a}; and the other of R^{3a} and R^{3b} is H, optionally each R^{a} substituent present in R^{3a} or R^{3b} is independently selected from the group consisting of: halo, C₁₋₄ alkoxy, and C₁₋₄ haloalkoxy.

In certain embodiments, R^{1c}, R^{2a}, and R^{2b} are each H; one of R^{3a} and R^{3b}, such as R^{3a}, is C₁₋₃ alkyl optionally substituted with from C₁₋₄ alkoxy; optionally one of R^{3a} and R^{3b}, such as R^{3a}, is —CH₂CH₂—OMe; and the other of R^{3a} and R^{3b} is H.

In certain embodiments, R^{1c}, R^{2a}, and R^{2b} are each H; one of R^{3a} and R^{3b}, such as R^{3a}, is C₁₋₃ alkyl optionally substituted with from 1-3 R^{a}; and the other of R^{3a} and R^{3b} is —F, optionally each R^{a} substituent present in R^{3a} or R^{3b} is independently selected from the group consisting of: halo, C₁₋₄ alkoxy, and C₁₋₄ haloalkoxy.

In certain embodiments, R^{1c}, R^{2a}, and R^{2b} are each H; one of R^{3a} and R^{3b}, such as R^{3a}, is C₁₋₃ alkyl optionally substituted with from 1-3 R^{a}; and the other of R^{3a} and R^{3b} is C₁₋₃ alkyl (e.g., methyl), optionally each R^{a} substituent present in R^{3a} or R^{3b} is independently selected from the group consisting of: halo, C₁₋₄ alkoxy, and C₁₋₄ haloalkoxy.

In certain embodiments, R^{1C}, R^{2a}, and R^{2b} are each H; one of R^{3a} and R^{3b}, such as R^{3a}, is C₃₋₆ (e.g., C₄) alkyl optionally substituted with from 1-3 R^{a}; and the other of R^{3a} and R^{3b} is H, —F, or C₁₋₃ alkyl (e.g., methyl), optionally each R^{a} substituent present in R^{3a} or R^{3b} is independently selected from the group consisting of: halo, C₁₋₄ alkoxy, and C₁₋₄ haloalkoxy.

In certain embodiments, R^{1c}, R^{2a}, and R^{2b} are each H, and one of R^{3a} and R^{3b}, such as R^{3a}, is —R^{g}, —(C₁₋₃ alkylene)-R^{g}, or —(C₁₋₃ alkylene)-O—R^{g}, optionally wherein the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
the other of R^{3a} and R^{3b} is H.

In some embodiments, R^{1c}, R^{2a}, and R^{2b} are each H; and R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

In certain embodiments, R^{1c}, R^{2a}, and R^{2b} are each H; and R^{3a} and R^{3b} taken together with the Ring B ring carbon atom to which each is attached form a fused C₃₋₆ (such as C₃ or C₄) cycloalkyl, wherein the fused cycloalkyl ring is optionally substituted with from 1-2 R^{c}.

In certain embodiments, R^{1c}, R^{2a}, and R^{2b} are each H; and R^{3a} and R^{3b} are independently selected C₁₋₃ alkyl.

In some embodiments, R^{1c} is H, and one of R^{2a} and R^{2b} (such as R^{2a}) and one of R^{3a} and R^{3b} (such as R^{3a}) taken together with the Ring B ring atoms to which each is attached, form a fused C₃₋₆ (such as C₃ or C₄) cycloalkyl which is optionally substituted with from 1-2 R^{c}; and the other of R^{2a} and R^{2b} and the other of R^{3a} and R^{3b} are each H.

In some embodiments, the other of R^{2a} and R^{2b} and the other of R^{3a} and R^{3b} are each H.

In the some embodiments, the other of R^{3a} and R^{3b} is C₁₋₃ alkyl. As non-limiting examples of the foregoing embodiments, the other of R^{3a} and R^{3b} is —CH₃, —CH₂CH₃.

In some embodiments, R^{1c} is H; one of R^{2a} and R^{2b} (such as R^{2a}) and one of R^{3a} and R^{3b} (such as R^{3a}) taken together with the Ring B ring atoms to which each is attached, form a fused C₃₋₆ (such as C₃ or C₄) cycloalkyl which is optionally substituted with from 1-2 R^{c}; and the other of R^{2a} and R^{2b} and the other of R^{3a} and R^{3b} are each H.

In some embodiments, R^{1c}, R^{2a}, R^{2b}, R^{3a}, and R^{3b} are each H.

In certain embodiments, the

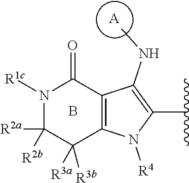

moiety is

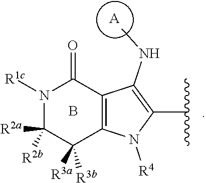

In certain embodiments, the

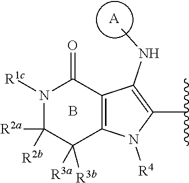

moiety is

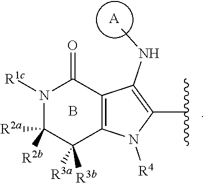

Variable R⁴, R⁷ and Ring A

In some embodiments, R⁴ is hydrogen.

In some embodiments, R⁷ is hydrogen.

In certain embodiments, R⁴ is hydrogen; and R⁴ is hydrogen.

In some embodiments, Ring A is

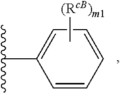

wherein each R^{cB} is an independently selected R^{c}; and m1 is 0, 1, 2, 3, or 4.

In certain of these embodiments, m1 is 1, 2, or 3. For example, m1 can be 1 or 2 (e.g., 2).

In certain embodiments, Ring A is

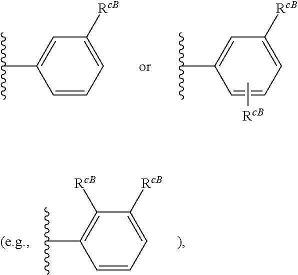

wherein each R^{cB} is an independently selected R^{c}.

As non-limiting examples, Ring A can be

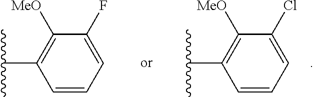 [SMILES: *c1cccc(F)c1OC ; *c1cccc(Cl)c1OC]

In certain embodiments, Ring A is selected from the group consisting of:

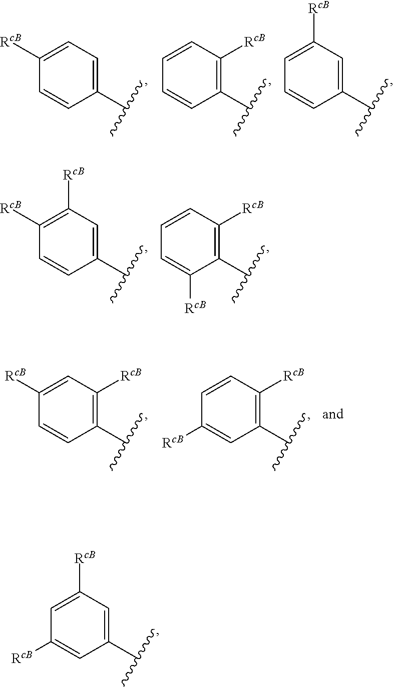

wherein each R^{cB} is an independently selected R^{c}.

In certain embodiments, each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

In certain embodiments, Ring A is

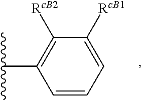

wherein R^{cB1} is R^{c}; and R^{cB2} is H or R^{c}, optionally wherein R^{cB1} and R^{cB2} are each independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

In certain embodiments (when Ring A is

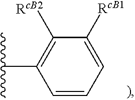

R^{cB1} is halo, such as —F or —Cl, such as —F.

In certain embodiments, R^{cB1} is C₁₋₃ alkyl or C₁₋₃ alkyl substituted with from 1-6 independently selected halo. For example, R^{cB1} can be methyl, —CHF₂, or —CF₃.

In certain embodiments, R^{cB2} is selected from the group consisting of: halo; —CN; C₁₋₄ alkoxy; C₁₋₄ haloalkoxy; C₁₋₃ alkyl; and C₁₋₃ alkyl substituted with from 1-6 independently selected halo. In certain of these embodiments, R^{cB2} is C₁₋₄ alkoxy or C₁₋₄ haloalkoxy.

In certain embodiments, R^{cB2} is selected from the group consisting of cyano; C₁₋₃ alkyl; and C₁₋₃ alkyl substituted with from 1-6 independently selected halo. For example, R^{cB2} can be cyano, methyl, ethyl, —CHF₂, —CF₃, or —CH₂CHF₂.

In some embodiments, Ring A is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 substituents independently selected from the group consisting of R^{c} and oxo.

In certain of these embodiments, Ring A is bicyclic heteroaryl including from 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 substituents independently selected from the group consisting of R^{c} and oxo.

In certain embodiments, Ring A is selected from the group consisting of:

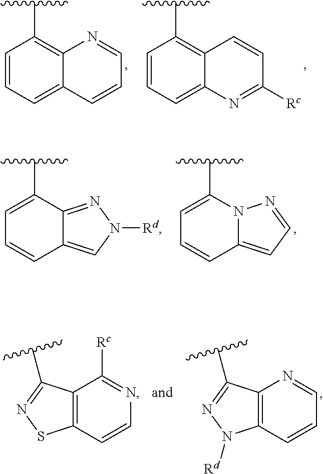

each of which is further optionally substituted with R^{c}.

Non-Limiting Combinations

In certain embodiments, the compound is a compound of Formula (I-a):

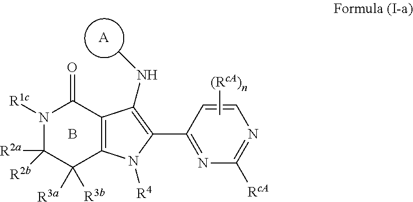

or a pharmaceutically acceptable salt thereof,
wherein: each R^{cA} is an independently selected R^{c}; and n is 0, 1, or 2.

In certain embodiments of Formula (I-a)

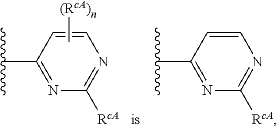

such as

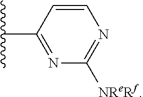

In certain of these foregoing embodiments, n is 0, and R^{cA} is C₁₋₃ alkyl optionally substituted with from 1-3 independently selected halo.

As a non-limiting example

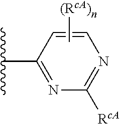

can be

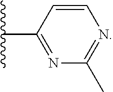 [SMILES: *c1ccnc(C)n1]

In certain embodiments of Formula (I-a)

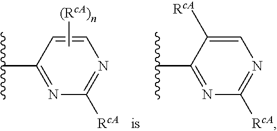

such as

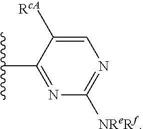

In certain embodiments of Formula (I-a), one of R^{3a} and R^{3b}, such as R^{3a}, is C₁₋₃ alkyl substituted with C₁₋₄ alkoxy; optionally wherein the other one of R^{3a} and R^{3b}, such as R^{3b} is H.

In certain embodiments of Formula (I-a), one of R^{3a} and R^{3b}, such as R^{3a}, is —CH₂OMe, —CH₂CH₂OMe, —CH(Me)CH₂OMe, —CH₂CH(Me)OMe, or -CH₂OEt; optionally wherein one of R^{3a} and R^{3b}, such as R^{3a} is —CH₂CH₂OMe.

In certain embodiments, the compound is a compound of Formula (I-b):

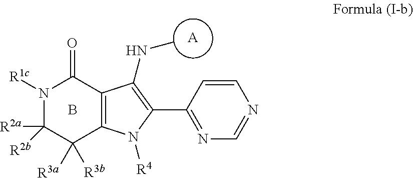

or a pharmaceutically acceptable salt thereof.

In certain embodiments, the compound is a compound of Formula (I-c):

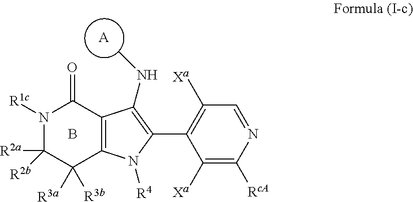

or a pharmaceutically acceptable salt thereof,
wherein: R^{cA} is an independently selected R^{c}.

In certain embodiments of Formula (I-c),

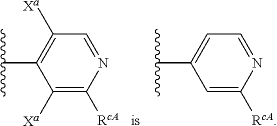

In certain embodiments, the compound is a compound of Formula (I-d):

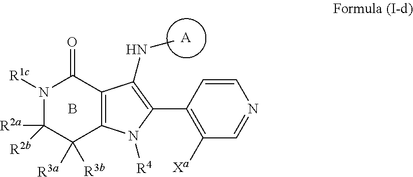

or a pharmaceutically acceptable salt thereof.

In certain embodiments of Formula (I-d), X^{a} is selected from H, —F, —Cl, C₁₋₆ alkyl, and C₁₋₃ alkyl substituted with from 1-3 independently selected halo. For example, X^{a} is —F. In certain embodiments of Formula (I-d), X^{a} is C₁₋₃ substituted with from 1-3 independently selected halo. As non-limiting examples of these foregoing embodiments of Formula (I-d), X^{a} is —CF₂H or —CF₃.

In certain embodiments, the compound is a compound of Formula (I-e):

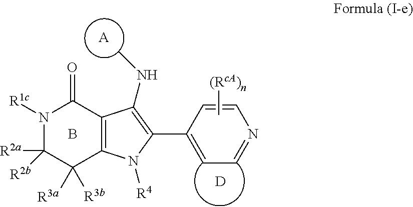

or a pharmaceutically acceptable salt thereof,
wherein: each R^{cA} is an independently selected R^{c};
n is 0, 1, or 2; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain embodiments of Formula (I-e), Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain of these embodiments,

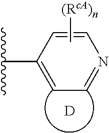

is selected from the group consisting of:

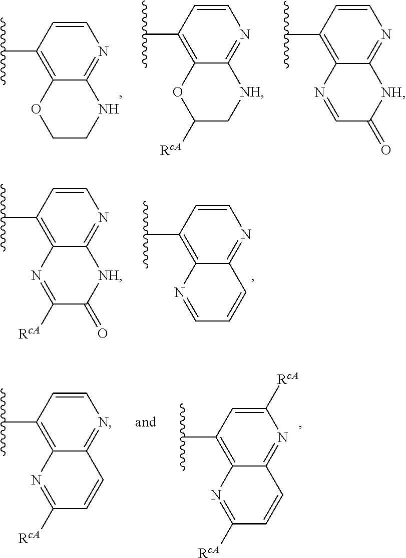

each further optionally substituted with R^{cA}, wherein each R^{cA} is an independently selected R^{c}.

In certain of these embodiments,

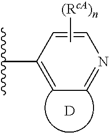

is selected from the group consisting of:

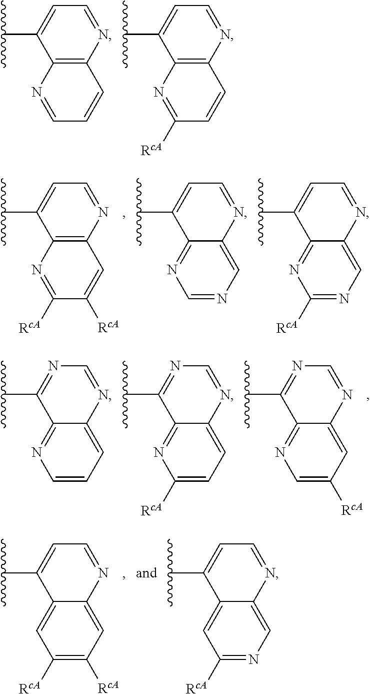

each further optionally substituted with R^{cA}, wherein each R^{cA} is an independently selected R^{c}.

In certain of the foregoing embodiments,

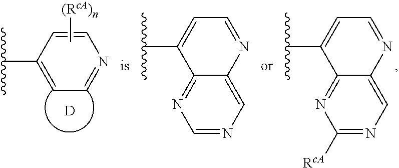

wherein R^{cA} is an independently selected R^{c}.

In certain of the foregoing embodiments,

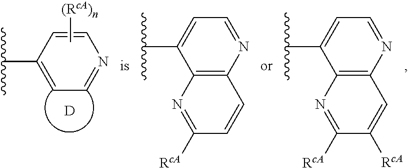

wherein each R^{cA} is an independently selected R^{c}.

In certain of the foregoing embodiments,

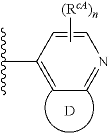

is selected from the group consisting of:

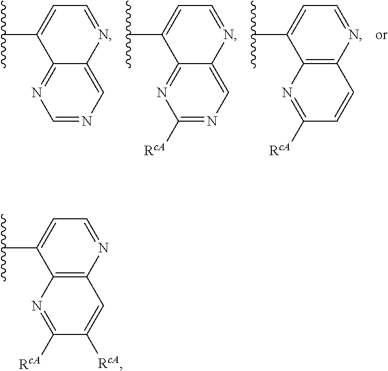

wherein:
each occurrence of R^{cA} is independently selected from the group consisting of: halo; NR^{e}R^{f}; C₁₋₄ alkoxy; C₁₋₄ haloalkoxy; C₁₋₃ alkyl; C₁₋₃ alkyl substituted with from 1-3 independently selected halo; C₁₋₃ alkyl substituted with C₁₋₄ alkoxy; and C₁₋₄ alkoxy substituted with C₁₋₄ alkoxy;
such as wherein each occurrence of R^{cA} is independently selected from the group consisting of: C₁₋₄ alkoxy; C₁₋₄ haloalkoxy; C₁₋₃ alkyl; and C₁₋₃ alkyl substituted with from 1-3 independently selected halo.

In certain embodiments of Formula (I-e), Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain of these embodiments,

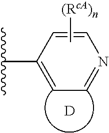

is selected from the group consisting of:

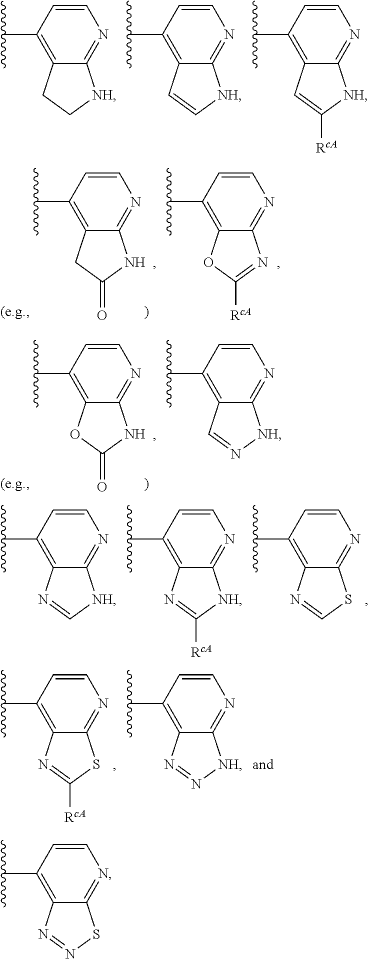

each further optionally substituted with R^{cA}, wherein each R^{cA} is an independently selected R^{c}.

In certain of these embodiments,

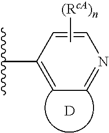

is selected from the group consisting of:

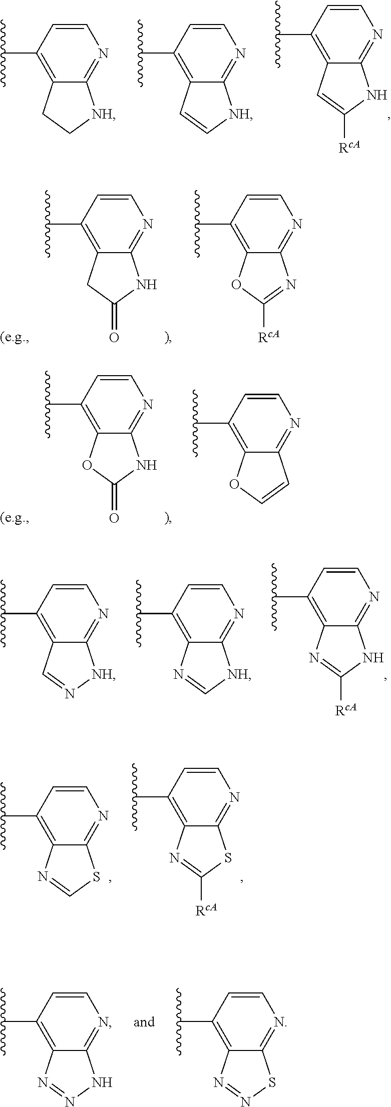

each further optionally substituted with R^{cA}, wherein each R^{cA} is an independently selected R^{c}.

In certain embodiments, the compound is a compound of Formula (I-f):

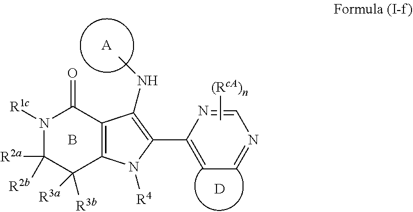

or a pharmaceutically acceptable salt thereof,
wherein: each R^{cA} is an independently selected R^{c};
n is 0 or 1; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain embodiments of Formula (I-f), Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain of these embodiments,

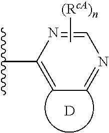

is selected from the group consisting of:

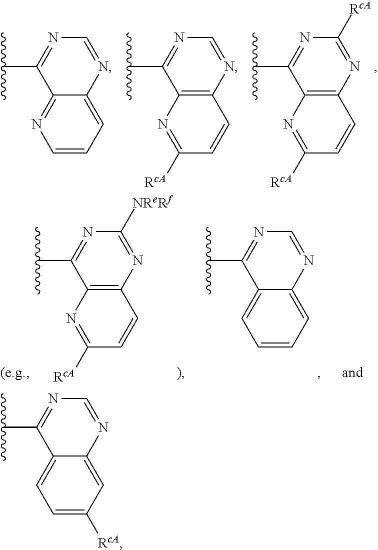

each further optionally substituted with R^{cA}, wherein each R^{cA} is an independently selected R^{c}.

In certain embodiments of Formula (I-f), Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain of these embodiments,

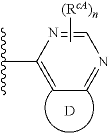

is selected from the group consisting of:

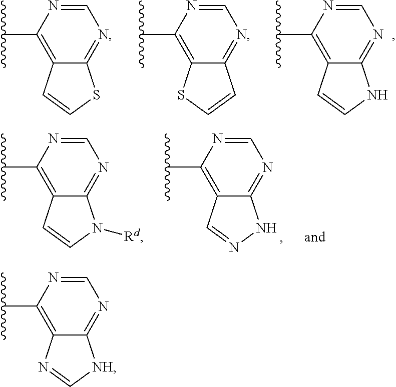

each further optionally substituted with R^{cA}, wherein each R^{cA} is an independently selected R^{c}.

In certain embodiments, the compound is a compound of Formula (I-g):

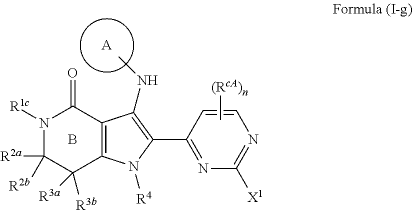

or a pharmaceutically acceptable salt thereof,
wherein: each R^{cA} is an independently selected R^{c}; and n is 0, 1, or 2.

In certain embodiments of Formula (I-g),

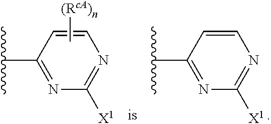

In certain embodiments, the compound is a compound of Formula (I-h):

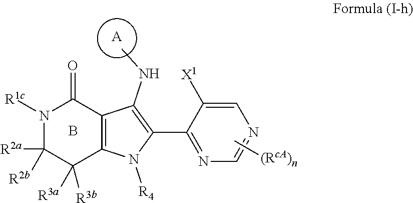

or a pharmaceutically acceptable salt thereof,
wherein: each R^{cA} is an independently selected R^{c}; and n is 0, 1, or 2.

In certain embodiments of Formula (I-h),

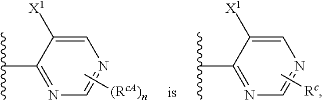

such as

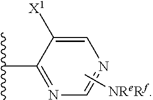

In certain embodiments, the compound is a compound of Formula (I-i):

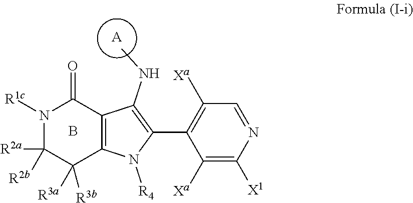

or a pharmaceutically acceptable salt thereof.

In certain embodiments of Formula (I-i), each X^{a} is H.

In certain embodiments the compound is a compound of Formula (I-j):

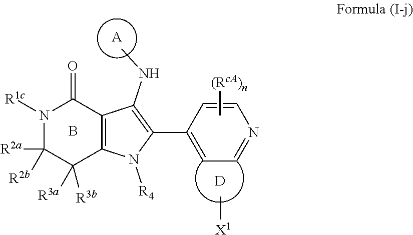

or a pharmaceutically acceptable salt thereof;
wherein n is 0, 1, or 2;
each R^{cA} is an independently selected R^{c}; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain embodiments of Formula (I-j), Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain of these embodiments,

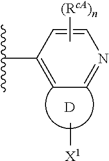

is selected from the group consisting of:

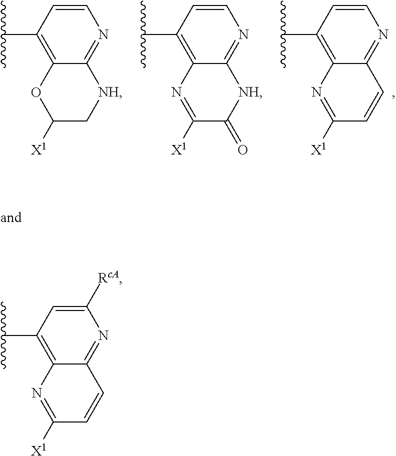

each further optionally substituted with R^{cA}, wherein each R^{cA} is an independently selected R^{c}.

In certain of the foregoing embodiments,

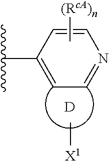

is selected from the group consisting of:

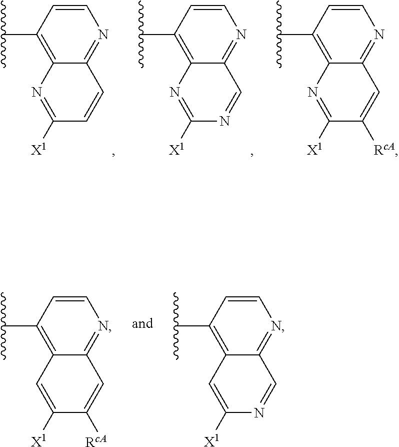

each of which is further optionally substituted with from 1-2 R^{cA}, wherein each R^{cA} is an independently selected R^{c}.

In certain of the foregoing embodiments,

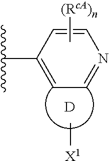

is selected from the group consisting of:

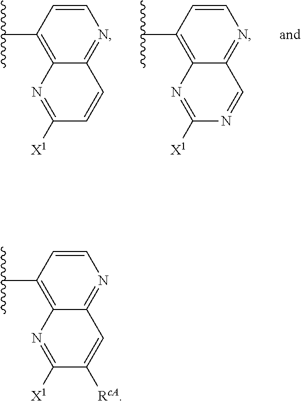

In certain embodiments of Formula (I-j), Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain of these embodiments,

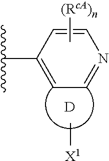

is selected from the group consisting of:

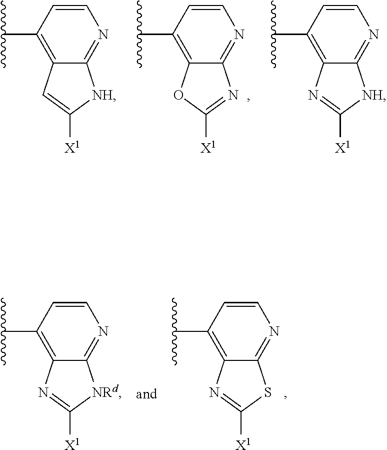

each further optionally substituted with R^{cA}, wherein each R^{cA} is an independently selected R^{c}.

In certain embodiments, the compound is a compound of Formula (I-k):

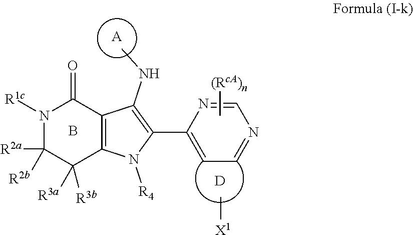

or a pharmaceutically acceptable salt thereof;
wherein n is 0 or 1;
each R^{cA} is an independently selected R^{c}; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{ca}.

In certain of these embodiments, Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain embodiments of Formula (I-k),

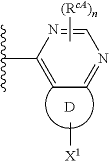

is selected from the group consisting of:

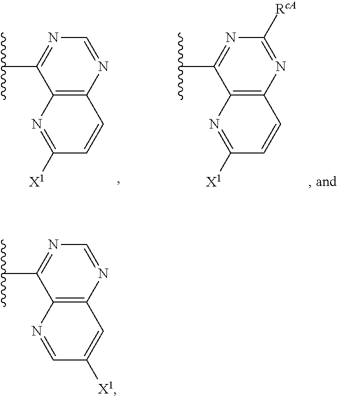

each further optionally substituted with R^{cA}, wherein each R^{cA} is an independently selected R^{c}.

In certain embodiments of Formula (I-k), Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

In certain embodiments of Formula (I-a), (I-b), (I-c), (I-d), (I-e), (I-f), (I-g), (I-h), (I-i), (I-j), or (I-k), each occurrence of R^{cA} is independently selected from the group consisting of: halo; cyano; C₁₋₁₀ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —NR^{e}R^{f}; —OH: —S(O)₁₋₂NR′R″; —C₁₋₄ thioalkoxy; —C(═O)(C₁₋₁₀ alkyl); —C(═O)O(C₁₋₄ alkyl); —C(═O)OH; and —C(═O)NR′R″.

In certain embodiments of Formula (I-a), (I-b), (I-c), (I-d), (I-e), (I-f), (I-g), (I-h), (I-i), (I-j), or (I-k), one occurrence of R^{cA} is —NR^{e}R^{f}.

In certain embodiments of Formula (I-a), (I-b), (I-c), (I-d), (I-e), (I-f), (I-g), (I-h), (I-i), (I-j), or (I-k), one occurrence of R^{cA} is —NH₂.

In certain embodiments of Formula (I-a), (I-b), (I-c), (I-d), (I-e), (I-f), (I-g), (I-h), (I-i), (I-j), or (I-k), one occurrence of R^{cA} is —NH(C₁₋₆ alkyl), wherein the C₁₋₆ alkyl is optionally substituted with from 1-3 substituents each independently selected from the group consisting of NR′R″, —OH, C₁₋₆ alkoxy, C₁₋₆ haloalkoxy, and halo. For example, one occurrence of R^{cA} can be —NHMe, —NHCH₂CF₃, —NHCH₂CH₂OH, or -NHiPr.

In certain embodiments of Formula (I-a), (I-b), (I-c), (I-d), (I-e), (I-f), (I-g), (I-h), (I-i), (I-j), or (I-k), one occurrence of R^{cA} is —NHC(═O)C₁₋₄ alkyl, such as NHC(═O)CH₃; or wherein one occurrence of R^{cA} is N(C₁₋₃ alkyl)₂ such as NMe₂.

In certain embodiments of Formula (I-a), (I-b), (I-c), (I-d), (I-e), (I-f), (I-g), (I-h), (I-i), (I-j), or (I-k), one occurrence of R^{cA} is C₁₋₄ alkoxy optionally substituted with C₁₋₄ alkoxy or CIA haloalkoxy. For example, one occurrence of R^{cA} can be OMe or OCH₂CH₂OMe. As another non-limiting example, R^{cA} can be C₁₋₄ haloalkoxy, such as —OCH₂CF₃.

In certain embodiments of Formula (I-a), (I-b), (I-c), (I-d), (I-e), (I-f), (I-g), (I-h), (I-i), (I-j), or (I-k), one occurrence of R^{ca} is C₁₋₄ thioalkoxy (e.g., SCH₃).

In certain embodiments of Formula (I-a), (I-b), (I-c), (I-d), (I-e), (I-f), (I-g), (I-h), (I-i), (I-j), or (I-k), one occurrence of R^{cA} is C₁₋₆ alkyl, such as methyl; or wherein one occurrence of R^{cA} is C₁₋₆ alkyl substituted with from 1-6 independently selected halo (e.g., R^{cA} can be —CF₃).

In certain embodiments of Formula (I-a), (I-b), (I-c), (I-d), (I-e), (I-f), (I-g), (I-h), (I-i), (I-j), or (I-k), one occurrence of R^{cA} is C₁₋₆ alkyl substituted with R^{a}, such as C₁₋₆ alkyl substituted with C₁₋₃ alkoxy or C(═O)NR′R″. For example, one occurrence of R^{cA} can be

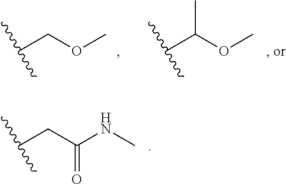

In certain embodiments of Formula (I-a), (I-b), (I-c), (I-d), (I-e), (I-f), (I-g), (I-h), (I-i), (I-j), or (I-k), one occurrence of R^{cA} is halo (e.g., —F).

In certain embodiments of Formula (I-a), (I-b), (I-c), (I-d), (I-e), (I-f), (I-g), (I-h), (I-i), (I-j), or (I-k), one occurrence of R^{cA} is —OH.

In certain embodiments of Formula (I-a), (I-b), (I-c), (I-d), (I-e), (I-f), (I-g), (I-h), (I-i), (I-j), or (I-k), one occurrence of R^{cA} is C(═O)NR′R″, such as C(═O)NHMe.

In Formula (I-a), (I-b), (I-c), (I-d), (I-e), (I-f), (I-g), (I-h), (I-i), (I-j), or (I-k), X¹ can be as defined anywhere herein. In certain embodiments, X¹ can be as defined in [AA1], [BB1], [CC1], [DD1], [EE1], or [FF1], infra:

[AA1]:

In certain embodiments of Formula (I-a), (I-b), (I-c), (I-d), (I-e), (I-f), (I-g), (I-h), (I-i), (I-j), or (I-k), X¹ is —(X²)ₘ-L¹-R⁵, wherein:
m is 0 or 1;
X² is —N(R^{N})— or —O—;
L¹ is a bond or C₁₋₆ alkylene optionally substituted with from 1-3 R^{a}; and
R⁵ is —R^{g}.

In certain embodiments of [AA1], R⁵ is phenyl optionally substituted with from 1-4 R^{c}, such as wherein R⁵ is phenyl optionally substituted with from 1-2 independently selected halo, such as —F.

In certain embodiments of [AA1], R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

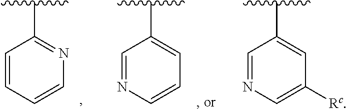

In certain embodiments of [AA1], R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

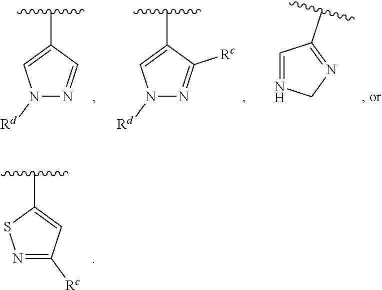

In certain embodiments of [AA1], R⁵ is C₃₋₁₀ cycloalkyl, such as C₃₋₆ cycloalkyl, optionally substituted with from 1-4 R^{c}, such as wherein R⁵ is cyclopropyl.

In certain embodiments of [AA1], R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R⁵ can be

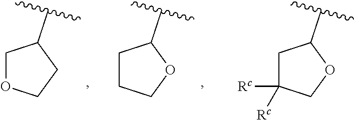

such as

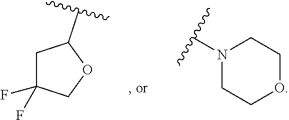 [SMILES: *C1CC(F)(F)CO1 ; ", or" ; *N1CCOCC1]

In certain embodiments of [AA1], m is 0.

In certain embodiments of [AA1], m is 1.

In certain embodiments of [AA1], X² is —N(R^{N})— (e.g., N(H)).

In certain embodiments of [AA1], X² is —O—.

In certain embodiments of [AA1], L¹ is a bond.

In certain embodiments of [AA1], L¹ is C₁₋₃ alkylene (e.g., —CH₂—, —CH₂CH₂—, or —CH(Me)-).

In certain embodiments of [AA1], L¹ is branched C₃₋₆ alkylene. For example, L¹ can be

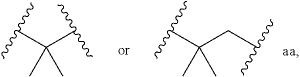

wherein aa is the point of attachment to R⁵.

[BB1]:

In certain embodiments of Formula (I-a), (I-b), (I-c), (I-d), (I-e), (I-f), (I-g), (I-h), (I-i), (I-j), or (I-k), X¹ is —X²-L¹-R⁵, wherein:
X² is —N(R^{N})C(═O)—*, —N(R^{N})S(O)₂—*, —N(R^{N})C(═O)O—*, or N(R^{N})C(═O)N(R^{N})*;
L¹ is a bond or C₁₋₆ alkylene optionally substituted with from 1-3 R^{a}; and
R⁵ is —R^{g}.

In certain embodiments of [BB1], R⁵ is phenyl optionally substituted with from 1-4 R^{c}, such as wherein R⁵ is phenyl optionally substituted with from 1-2 independently selected halo, such as —F.

In certain embodiments of [BB1], R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

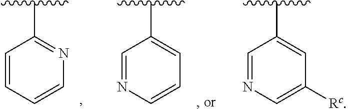

In certain embodiments of [BB1], R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

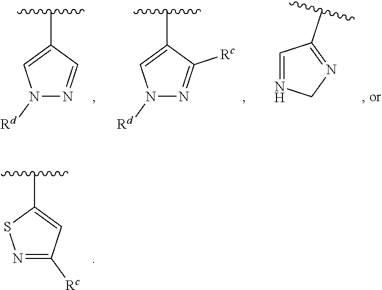

In certain embodiments of [BB1], R⁵ is C₃₋₁₀ cycloalkyl, such as C₃₋₆ cycloalkyl, optionally substituted with from 1-4 R^{c}, such as wherein R⁵ is cyclopropyl.

In certain embodiments of [BB1], R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R⁵ can be

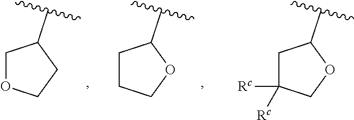

such as

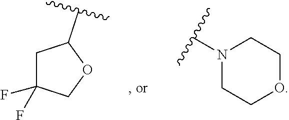 [SMILES: *C1CC(F)(F)CO1 ; ", or" ; *N1CCOCC1]

In certain embodiments of [BB1], X² is —N(R^{N})C(═O)—* (e.g., —N(H)C(═O)—*).

In certain embodiments of [BB1], X² is —N(R^{N})S(O)₂—, such as —N(H)S(O)₂—*.

In certain embodiments of [BB1], X² is —N(R^{N})C(═O)O—*, or —N(R^{N})C(═O)N(R^{N})—* (e.g., —N(H)C(═O)O—*; e.g., —N(H)C(═O)N(H)—*).

In certain embodiments of [BB1], L¹ is a bond.

In certain embodiments of [BB1], L¹ is C₁₋₃ alkylene (e.g., —CH₂—, —CH₂CH₂—, or CH(Me)-).

In certain embodiments of [BB1], L¹ is branched C₃₋₆ alkylene. For example, L¹ can be

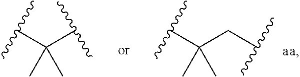

wherein aa is the point of attachment to R⁵.

[CC1]:

In certain embodiments of Formula (I-a), (I-b), (I-c), (I-d), (I-e), (I-f), (I-g), (I-h), (I-i), (I-j), or (I-k), X¹ is —X²-L¹-R⁵, wherein:
X² is

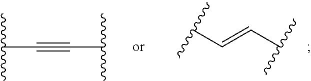

L¹ is a bond or C₁₋₆ alkylene optionally substituted with from 1-3 R^{a}; and
R⁵ is —R^{g}.

In certain embodiments of [CC1], R⁵ is phenyl optionally substituted with from 1-4 R^{c}, such as wherein R⁵ is phenyl optionally substituted with from 1-2 independently selected halo, such as —F.

In certain embodiments of [CC1], R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

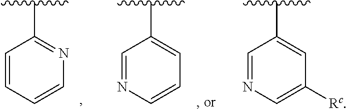

In certain embodiments of [CC1], R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

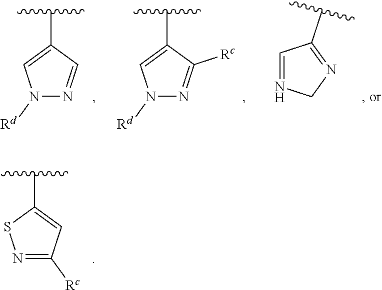

In certain embodiments of [CC1], R⁵ is C₃₋₁₀ cycloalkyl, such as C₃₋₆ cycloalkyl, optionally substituted with from 1-4 R^{c}, such as wherein R⁵ is cyclopropyl.

In certain embodiments of [CC1], R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R⁵ can be

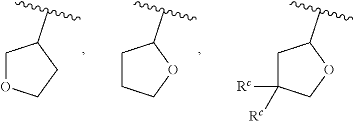

such as

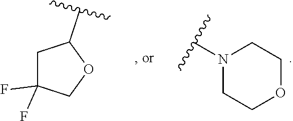 [SMILES: *C1CC(F)(F)CO1 ; *N1CCOCC1]

In some embodiments of [CC1], X² is

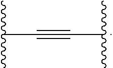 [SMILES: *C#C*]

In some embodiments of [CC1], X² is

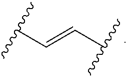 [SMILES: */C=C/*]

In certain embodiments of [CC1], L¹ is a bond.

In certain embodiments of [CC1], L¹ is C₁₋₃ alkylene (e.g., —CH₂—, —CH₂CH₂—, or —CH(Me)-).

In certain embodiments of [CC1], L¹ is branched C₃₋₆ alkylene. For example, L¹ can be

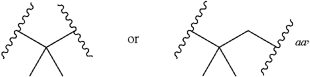

wherein aa is the point of attachment to R⁵.

[DD1]:

In certain embodiments of Formula (I-a), (I-b), (I-c), (I-d), (I-e), (I-f), (I-g), (I-h), (I-i), (I-j), or (I-k), X¹ is —(X²)ₘ-L¹-R⁵, wherein:
m is 0 or 1;
X² is —N(R^{N})— or —O—;
L¹ is a bond or C₁₋₆ alkylene optionally substituted with from 1-3 R^{a}; and
R⁵ is —R^{g2}-R^{Y}.

In certain embodiments of [DD1], the —R^{g2} group present in R⁵ is 1,3-phenylene or 1,4-phenylene, each optionally substituted with from 1-4 R^{c}, such as wherein —R^{g2} is

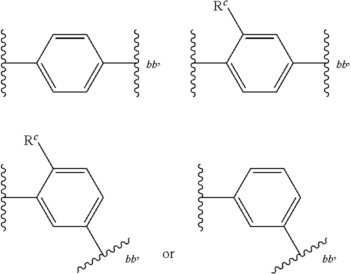

wherein bb is the point of attachment to R^{Y}.

In certain embodiments of [DD1], the R^{Y} group present in R⁵ is —R^{g}.

In certain embodiments of [DD1], the R^{Y} group present in R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}. For example, R^{Y} can be

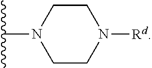

In certain embodiments of [DD1], X² is —N(R^{N})— (e.g., N(H)).

In certain embodiments of [DD1], X² is —O—.

In certain embodiments of [DD1], L¹ is a bond.

In certain embodiments of [DD1], L¹ is C₁₋₃ alkylene (e.g., —CH₂—, —CH₂CH₂—, or —CH(Me)-).

In certain embodiments of [DD1], L¹ is branched C₃₋₆ alkylene. For example, L¹ can be

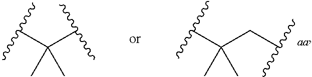

wherein aa is the point of attachment to R⁵.

[EE1]:

In certain embodiments of Formula (I-a), (I-b), (I-c), (I-d), (I-e), (I-f), (I-g), (I-h), (I-i), (I-j), or (I-k), X¹ is —X²-L¹-R⁵, wherein:
X² is —N(R^{N})—, —O—, —N(R^{N})C(═O)—*, —N(R^{N})S(O)₂—, —N(R^{N})C(═O)O—*, or —N(R^{N})C(═O)N(R^{N})—*;
L¹ is C₁₋₆ alkylene optionally substituted with from 1-3 R^{a}; and
R⁵ is H, halo, C₁₋₆ alkoxy optionally substituted with from 1-3 R^{a}, or —OH.

In certain embodiments of [EE1], R⁵ is H.

In certain embodiments of [EE1], R⁵ is halo (e.g., —F).

In certain embodiments of [EE1], R⁵ is C₁₋₆ alkoxy optionally substituted with from 1-3 R^{a}, such as wherein R⁵ is C₁₋₃ alkoxy such as methoxy.

In certain embodiments of [EE1], R⁵ is —OH.

In certain embodiments of [EE1], X² is —N(R^{N})— (e.g., N(H)).

In certain embodiments of [EE1], X² is —O—.

In certain embodiments of [EE1], X² is —N(R^{N})C(═O)—* (e.g., —N(H)C(═O)—*).

In certain embodiments of [EE1], X² is —N(R^{N})S(O)₂—, such as —N(H)S(O)₂—*.

In certain embodiments of [EE1], X² is —N(R^{N})C(═O)O—*, or —N(R^{N})C(═O)N(R^{N})—* (e.g., —N(H)C(═O)O—*; e.g., —N(H)C(═O)N(H)—*).

In certain embodiments of [EE1], L¹ is C₁₋₃ alkylene (e.g., —CH₂—, —CH₂CH₂—, or CH(Me)-).

In certain embodiments of [EE1], L¹ is branched C₃₋₆ alkylene. For example, L¹ can be

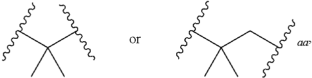

wherein aa is the point of attachment to R⁵.

[FF1]:

In certain embodiments of Formula (I-a), (I-b), (I-c), (I-d), (I-e), (I-f), (I-g), (I-h), (I-i), (I-j), or (I-k), X¹ is -L¹-R⁵, wherein L¹ is C₁₋₆ alkylene optionally substituted with from 1-3 R^{a}; and R⁵ is -L⁵-R^{g}.

In certain embodiments of [FF1], R⁵ is —O—R^{g}.

In certain embodiments of [FF1], R⁵ is —O-(phenyl), wherein the phenyl is optionally substituted with from 1-2 R^{c}.

In certain embodiments of [FF1], L¹ is C₁₋₃ alkylene (e.g., —CH₂—, —CH₂CH₂—, or —CH(Me)-).

In certain embodiments of Formula (I-a), (I-b), (I-c), (I-d), (I-e), (I-f), (I-g), (I-h), (I-i), (I-j), or (I-k), R^{1c} is H.

In certain embodiments of Formula (I-a), (I-b), (I-c), (I-d), (I-e), (I-f), (I-g), (I-h), (I-i), (I-j), or (I-k), R^{2a} and R^{2b} are both H.

In certain embodiments of Formula (I-a), (I-b), (I-c), (I-d), (I-e), (I-f), (I-g), (I-h), (I-i), (I-j), or (I-k), R^{2a} is a substituent that is other than H. In certain of these embodiments, R^{a} is C₁₋₆ alkyl which is optionally substituted with from 1-6 R^{a}, such as wherein R^{2a} is C₁₋₃ alkyl, such as methyl or ethyl.

In certain embodiments of Formula (I-a), (I-b), (I-c), (I-d), (I-e), (I-f), (I-g), (I-h), (I-i), (I-j), or (I-k), R^{2b} is H.

In certain embodiments of Formula (I-a), (I-b), (I-c), (I-d), (I-e), (I-f), (I-g), (I-h), (I-i), (I-j), or (I-k), R^{3a} and R^{3b} are both H.

In certain embodiments of Formula (I-a), (I-b), (I-c), (I-d), (I-e), (I-f), (I-g), (I-h), (I-i), (I-j), or (I-k), R^{3a} is a substituent that is other than H. In certain of these embodiments, R^{3a} is C₁₋₆ alkyl which is optionally substituted with from 1-6 R^{a}, such as wherein R^{3a} is C₁₋₃ alkyl, such as methyl or ethyl.

In certain of foregoing embodiments of Formula (I-a), (I-b), (I-c), (I-d), (I-e), (I-f), (I-g), (I-h), (I-i), (I-j), or (I-k), R^{3a} is C₁₋₃ alkyl substituted with from 1-3 independently selected halo. As non-limiting examples of the foregoing embodiments, R^{3a} is —CH₂F, —CHF₂, —CF₃, —CH₂CHF₂, or —CH₂CH₂F.

In certain embodiments of Formula (I-a), (I-b), (I-c), (I-d), (I-e), (I-f), (I-g), (I-h), (I-i), (I-j), or (I-k), R^{3a} is C₁₋₃ alkyl substituted with C₁₋₄ alkoxy, C₁₋₄ haloalkoxy, or NR^{e}R^{f} Non-limiting examples of R^{3a} in these embodiments include —CH₂OMe, —CH₂CH₂OMe, —CH(Me)CH₂OMe, —CH₂CH(Me)OMe, -CH₂OEt, —CH₂CH₂OCHF₂, —CH₂NR^{e}R^{f} (e.g., —CH₂N(CF₃)Me), or —CH₂CH₂NR^{e}R^{f} (e.g., —CH₂CH₂NMe₂).

In certain embodiments of Formula (I-a), (I-b), (I-c), (I-d), (I-e), (I-f), (I-g), (I-h), (I-i), (I-j), or (I-k), R^{3a} is C₁₋₃ alkyl substituted with C₁₋₄ alkoxy, C₁₋₄ haloalkoxy, or NR^{e}R^{f} and further substituted with from 1-3 independently selected halo. In certain of these embodiments, R^{3a} is C₁₋₃ alkyl substituted with C₁₋₄ alkoxy and further substituted with from 1-3 independently selected halo. Non-limiting examples of R^{3a} in these embodiments include:

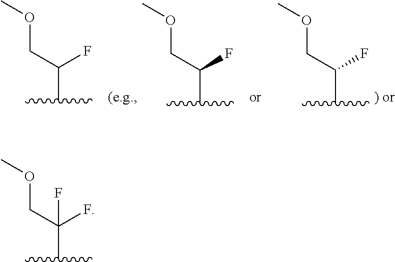

In certain embodiments of Formula (I-a), (I-b), (I-c), (I-d), (I-e), (I-f), (I-g), (I-h), (I-i), (I-j), or (I-k), R^{3a} is C₃₋₆ alkyl substituted with C₁₋₄ alkoxy, C₁₋₄ haloalkoxy, or NR^{e}R^{f}. In certain of these embodiments, R^{3a} is branched C₃₋₆ alkyl substituted with C₁₋₄ alkoxy, C₁₋₄ haloalkoxy, or NR^{e}R^{f}. In certain of the foregoing embodiments, R^{3a} is branched C₃₋₆ alkyl substituted with C₁₋₄ alkoxy. For example, R^{3a} can be

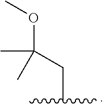 [SMILES: *CC(C)(C)OC]

In certain of the foregoing embodiments of Formula (I-a), (I-b), (I-c), (I-d), (I-e), (I-f), (I-g), (I-h), (I-i), (I-j), or (I-k), R^{3a} is selected from the group consisting of:
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
C₃₋₆ cycloalkyl optionally substituted with from 1-4 R^{c}.

In certain of the foregoing embodiments of Formula (I-a), (I-b), (I-c), (I-d), (I-e), (I-f), (I-g), (I-h), (I-i), (I-j), or (I-k), R^{3a} is —(C₁₋₃ alkylene)-R^{g} or —(C₁₋₃ alkylene)-O—R^{g}, and optionally the R^{g} group of R^{3a} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

In certain embodiments of Formula (I-a), (I-b), (I-c), (I-d), (I-e), (I-f), (I-g), (I-h), (I-i), (I-j), or (I-k), R^{3a} is —CH₂—R^{g}, or —CH₂CH₂R^{g}, wherein R^{g} is 1,4-dioxanyl.

In certain embodiments of Formula (I-a), (I-b), (I-c), (I-d), (I-e), (I-f), (I-g), (I-h), (I-i), (I-j), or (I-k), R^{3a} is-(L^{g})_{g}-R^{W}.

In certain embodiments of Formula (I-a), (I-b), (I-c), (I-d), (I-e), (I-f), (I-g), (I-h), (I-i), (I-j), or (I-k), R^{3a} is-CH₂CH₂—R^{W}, wherein the R^{W} group is C(═O)—CH═CH₂, or —NHC(═O)—CH═CH₂.

As a non-limiting example of certain embodiments of Formula (I-a), (I-b), (I-c), (I-d), (I-e), (I-f), (I-g), (I-h), (I-i), (I-j), or (I-k), R^{a} is

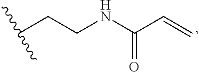 [SMILES: *CCNC(=O)C=C]

such as

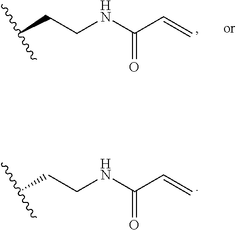

In certain embodiments of Formula (I-a), (I-b), (I-c), (I-d), (I-e), (I-f), (I-g), (I-h), (I-i), (I-j), or (I-k), R^{3a} is -(L^{g})_{g}-R^{g2}-R^{W}.

In certain embodiments of Formula (I-a), (I-b), (I-c), (I-d), (I-e), (I-f), (I-g), (I-h), (I-i), (I-j), or (I-k), R^{3a} is —CH₂—R^{g2}-R^{W}, wherein the R^{g2} group is

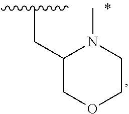 [SMILES: *CC1COCCN1C]

such as

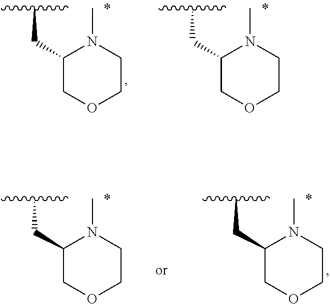

wherein the waveline represents the point of attachment to —CH₂— and the asterisk represents the point of attachment to R^{W}; and optionally the R^{W} group is C(═O)—CH═CH₂.

As a non-limiting example of certain embodiments of Formula (I-a), (I-b), (I-c), (I-d), (I-e), (I-f), (I-g), (I-h), (I-i), (I-j), or (I-k), R^{3a} can be

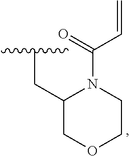 [SMILES: *CC1COCCN1C(=O)C=C]

such as

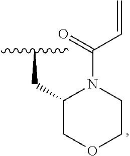

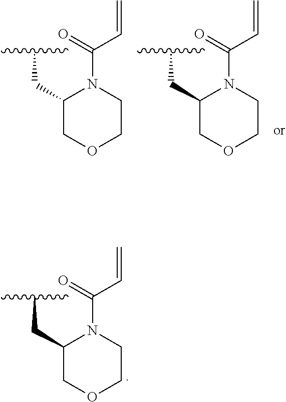

In certain embodiments of Formula (I-a), (I-b), (I-c), (I-d), (I-e), (I-f), (I-g), (I-h), (I-i), (I-j), or (I-k), R^{3b} is H.

In certain embodiments of Formula (I-a), (I-b), (I-c), (I-d), (I-e), (I-f), (I-g), (I-h), (I-i), (I-j), or (I-k), R^{3b} is C₁₋₃ alkyl. As non-limiting examples of the foregoing embodiments, R^{3b} is methyl, ethyl, or propyl. For example, R^{3b} is methyl.

In certain embodiments of Formula (I-a), (I-b), (I-c), (I-d), (I-e), (I-f), (I-g), (I-h), (I-i), (I-j), or (I-k), R^{3b} is H.

In certain embodiments of Formula (I-a), (I-b), (I-c), (I-d), (I-e), (I-f), (I-g), (I-h), (I-i), (I-j), or (I-k), R^{3b} is halo. For example, R^{3b} can be —F.

In certain embodiments of Formula (I-a), (I-b), (I-c), (I-d), (I-e), (I-f), (I-g), (I-h), (I-i), (I-j), or (I-k), R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

In certain of the foregoing embodiments of Formula (I-a), (I-b), (I-c), (I-d), (I-e), (I-f), (I-g), (I-h), (I-i), (I-j), or (I-k), R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

In certain embodiments, R^{3a} and R^{3b}, together with the Ring B ring atom to which each is attached, form a fused C₃₋₆ cycloalkyl, wherein the fused C₃₋₆ cycloalkyl is optionally substituted with from 1-2 R^{c}.

As non-limiting examples of the foregoing embodiments, R^{3a} and R^{3b}, together with the Ring B ring atom to which each is attached, form

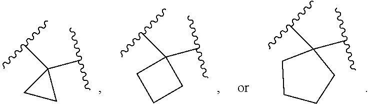 [SMILES: *C1(*)CC1 ; *C1(*)CCC1 ; *C1(*)CCCC1]

In certain embodiments of Formula (I-a), (I-b), (I-c), (I-d), (I-e), (I-f), (I-g), (I-h), (I-i), (I-j), or (I-k), R^{3a} and R^{3b}, together with the Ring B ring atom to which each is attached, form:

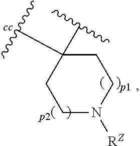

which is optionally substituted with from 1-2 substituents independently selected from the group consisting of oxo and R^{c}, wherein:
p1 and p2 are independently 0, 1, or 2;
R^{Z} is H, R^{d}, C(═O)—W, or S(O)₂W: and
cc represents the point of attachment to C(R^{2a}R^{2b}).

In certain embodiments of Formula (I-a), (I-b), (I-c), (I-d), (I-e), (I-f), (I-g), (I-h), (I-i), (I-j), or (I-k). R^{3a} and R^{3b}, together with the Ring B ring atom to which each is attached, form

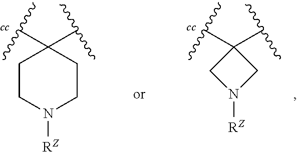

wherein R^{Z} is H, R^{d}, C(═O)—W, or S(O)₂W; and cc represents the point of attachment to C(R^{2a}R^{2b}).

In certain embodiments of Formula (I-a), (I-b), (I-c), (I-d), (I-e), (I-f), (I-g), (I-h), (I-i), (I-j), or (I-k), R^{3a} and R^{3b}, together with the Ring B ring atom to which each is attached, form a fused ring selected from the group consisting of:

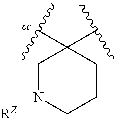

such as

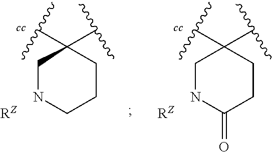

such as

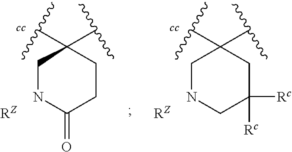

such as

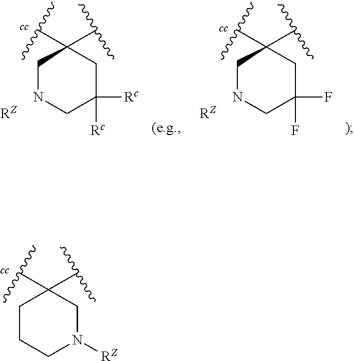

such as

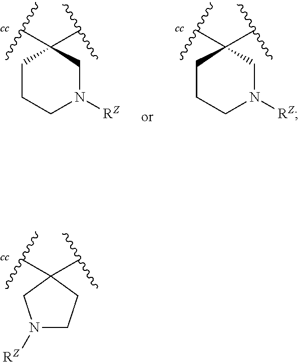

such as

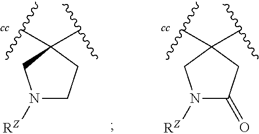

such as

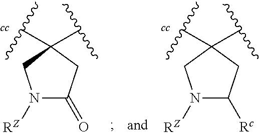

such as

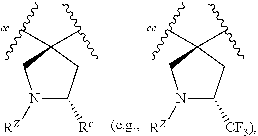

wherein R^{Z} is H, R^{d}, C(═O)—W, or S(O)₂W; and cc represents the point of attachment to C(R^{2a}R^{2b}).

In certain embodiments, R^{Z} is H. In certain embodiments, R^{Z} is C₁₋₆ alkyl optionally substituted with from 1-3 independently selected R^{a}. In certain embodiments, R^{Z} is C(═O)—W or S(O)₂W, optionally wherein W is C₂₋₄ alkenyl.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H; and R^{3a} and R^{3b} taken together with the Ring B ring carbon atom to which each is attached form a fused C₃₋₆ (such as C₃ or C₄) cycloalkyl, wherein the fused cycloalkyl ring is optionally substituted with from 1-2 R^{c}.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H; and R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

In certain embodiments of Formula (I-a), (I-b), (I-c), (I-d), (I-e), (I-f), (I-g), (I-h), (I-i), (I-j), or (i-k), one of R^{2a} and R^{2b} (such as R^{2a}) and one of R^{3a} and R^{3b} (such as R^{3a}) taken together with the Ring B ring atoms to which each is attached, form a fused saturated or unsaturated ring of 3-12 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo and R^{c}.

In certain of the foregoing embodiments, one of R^{2a} and R^{2b} (such as R^{2a}) and one of R^{3a} and R^{3b} (such as R^{3a}) taken together with the Ring B ring atoms to which each is attached, form a fused saturated ring of 3-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 3-8 ring atoms is optionally substituted with from 1-4 substituents independently selected from the group consisting of oxo and R^{c}.

In certain of these foregoing embodiments, one of R^{2a} and R^{2b} (such as R^{2a}) and one of R^{3a} and R^{3b} (such as R^{3a}) taken together with the Ring B ring atoms to which each is attached, form a fused C₃₋₆ cycloalkyl which is optionally substituted with from 1-2 R^{c}.

As non-limiting examples of the foregoing embodiments, one of R^{2a} and R^{2b} (such as R^{2a}) and one of R^{3a} and R^{3b} (such as R^{3a}) taken together with the Ring B ring atoms to which each is attached, form a fused cyclopropyl or cyclobutyl ring, e.g.,

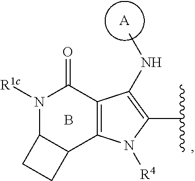

such as

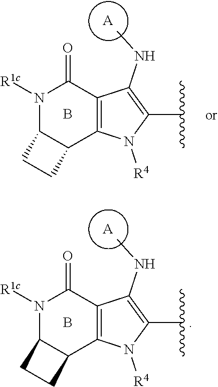

In certain embodiments of Formula (I-a), (I-b), (I-c), (I-d), (I-e), (I-f), (I-g), (I-h), (I-i), (I-j), or (I-k), R^{1c} is H; R^{2a} and R^{3a} combine to form a double bond between the Ring B atoms to which each is attached; and R^{2b} is H; and R^{3b} is -(L^{g})_{g}-R^{g}.

In certain embodiments of Formula (I-a), (I-b), (I-c), (I-d), (I-e), (I-f), (I-g), (I-h), (I-i), (I-j), or (I-k), R^{1c} is H; R^{2a} and R^{3a} combine to form a double bond between the Ring B atoms to which each is attached; and R^{2b} is H; and R^{3b} is

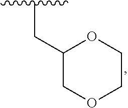 [SMILES: *CC1COCCO1]

such as

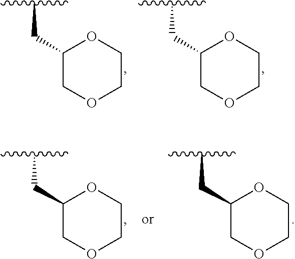

In certain of these foregoing embodiments, R^{1c} is H, and R^{2b} and R^{3b} are each H.

In certain embodiments of Formula (I-a), (I-b), (I-c), (I-d), (I-e), (I-f), (I-g), (I-h), (I-i), (I-j), or (I-k), R^{2b} and R^{3b} are each H.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H, and R^{3a} is C₁₋₃ alkyl optionally substituted with from 1-3 R^{a}.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H; R^{3a}, is C₁₋₃ alkyl optionally substituted with from 1-3 R^{a}; and R^{3b} is H, optionally each R^{a} substituent present in R^{3a} is independently selected from the group consisting of: halo, C₁₋₄ alkoxy, and C₁₋₄ haloalkoxy.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H; and R^{3a} and R^{3b} are independently selected C₁₋₃ alkyl.

In certain embodiments of Formula (I-a), (I-b), (I-c), (I-d), (I-e), (I-f), (I-g), (I-h), (I-i), (I-j), or (I-k), R^{1c} is H; R^{2a} and R^{3a} taken together with the Ring B ring atoms to which each is attached, form a fused C₃₋₆ (e.g., C₃ or C₄) cycloalkyl which is optionally substituted with from 1-2 R^{c}; and R^{2b} and R^{3b} are each H.

In certain embodiments of Formula (I-a), (I-b), (I-c), (I-d), (I-e), (I-f), (I-g), (I-h), (I-i), (I-j), or (I-k), R^{1c}, R^{2a}, R^{2b}, R^{3a}, and R^{3b} are each H.

In certain of these foregoing embodiments, R^{3b} is H, and each optionally present R^{a} substituent in R^{3a} is independently selected from the group consisting of: halo, C₁₋₄ alkoxy, and C₁₋₄ haloalkoxy.

In certain of embodiments, R^{3b} is —F, and each optionally present R^{a} substituent in R^{3a} is independently selected from the group consisting of: halo, C₁₋₄ alkoxy, and C₁₋₄ haloalkoxy.

In certain of embodiments, R^{3b} is C₁₋₃ alkyl (e.g., methyl), and each optionally present R^{a} substituent in R^{3a} is independently selected from the group consisting of: halo, C₁₋₄ alkoxy, and C₁₋₄ haloalkoxy.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H; R^{3a}, is —R^{g}, —(C₁₋₃ alkylene)-R^{g}, or —(C₁₋₃ alkylene)-O—R^{g},
optionally wherein the R^{g} group of R^{3a} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
R^{3b} is H.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H; and R^{3a} is C₁₋₃ alkyl optionally substituted with from 1-3 R^{a}; and R^{3b} is H, optionally each R^{a} substituent present in R^{3a} is independently selected from the group consisting of: halo, C₁₋₄ alkoxy, and C₁₋₄ haloalkoxy.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H and R^{3a}, is —R^{g}, —(C₁₋₃ alkylene)-R^{g}, or —(C₁₋₃ alkylene)-O—R^{g},
optionally wherein the R^{g} group of R^{3a} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
R^{3b} is H.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H, and R^{3a} and R^{3b} taken together with the Ring B ring carbon atom to which each is attached form a fused C₃₋₆ (such as C₃ or C₄) cycloalkyl, wherein the fused cycloalkyl ring is optionally substituted with from 1-2 R^{c}.

In certain embodiments of Formula (I-a), (I-b), (I-c), (I-d), (I-e), (I-f), (I-g), (I-h), (I-i), (I-j), or (I-k), R^{1c}, R^{2a}, and R^{2b} are each H, and R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

In certain embodiments of Formula (I-a), (I-b), (I-c), (I-d), (I-e), (I-f), (I-g), (I-h), (I-i), (I-j), or (I-k), R^{1C} is H, and R^{2a} and R^{3a} taken together with the Ring B ring atoms to which each is attached, form a fused C₃₋₆ (e.g., C₃ or C₄) cycloalkyl which is optionally substituted with from 1-2 R^{c}, and R^{2b} and R^{3b} are each H.

In certain embodiments of Formula (I-a), (I-b), (I-c), (I-d), (I-e), (I-f), (I-g), (I-h), (I-i), (I-j), or (I-k), R^{1c}, R^{2a}, R^{2b}, R^{3a}, and R^{3b} are each H.

In certain embodiments of Formula (I-a), (I-b), (I-c), (I-d), (I-e), (I-f), (I-g), (I-h), (I-i), (I-j), or (I-k), R⁴ is H.

In certain embodiments of Formula (I-a), (I-b), (I-c), (I-d), (I-e), (I-f), (I-g), (I-h), (I-i), (I-j), or (I-k), Ring A is

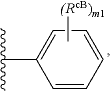

wherein each R^{cB} is an independently selected R^{c}; and m1 is 0, 1, 2, 3, or 4. In certain of these embodiments, m1 is 1, 2, or 3, such as 1 or 2.

In certain embodiments of Formula (I-a), (I-b), (I-c), (I-d), (I-e), (I-f), (I-g), (I-h), (I-i), (I-j), or (I-k), Ring A is

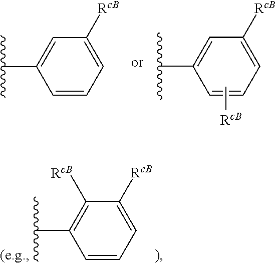

wherein each R^{cB} is an independently selected R^{c}.

As non-limiting example of certain embodiments of Formula (I-a), (I-b), (I-c), (I-d), (I-e), (I-f), (I-g), (I-h), (I-i), (I-j), or (I-k), Ring A can be

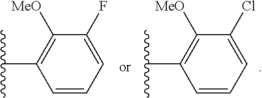

In certain embodiments of Formula (I-a), (I-b), (I-c), (I-d), (I-e), (I-f), (I-g), (I-h), (I-i), (I-j), or (I-k), Ring A is selected from the group consisting of:

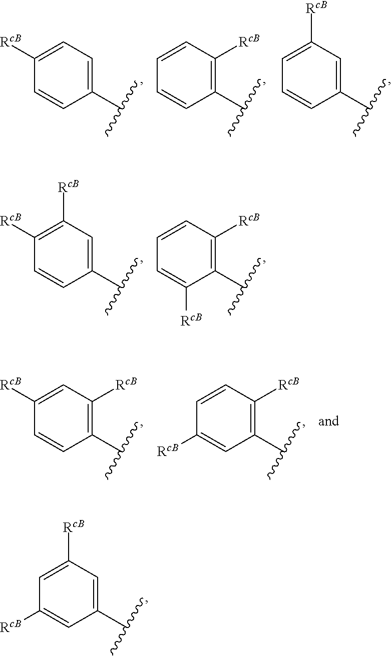

wherein each RCE is an independently selected R^{c}.

In certain embodiments, each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

In certain embodiments of Formula (I-a), (I-b), (I-c), (I-d), (I-e), (I-f), (I-g), (I-h), (I-i), (I-j), or (I-k), Ring A is bicyclic heteroaryl including from 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 substituents independently selected from the group consisting of R^{c} and oxo, such as wherein: Ring A is selected from the group consisting of:

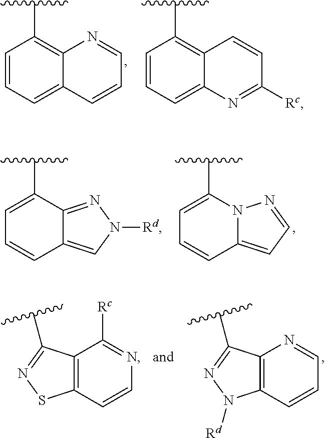

each of which is further optionally substituted with R^{c}.

In certain embodiments of Formula (I-a), (I-b), (I-c), (I-d), (I-e), (I-f), (I-g), (I-h), (I-i), (I-j), or (I-k), the

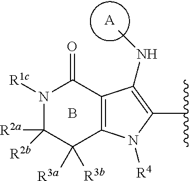

moiety is

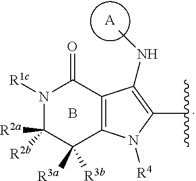

In certain embodiments of Formula (I-a), (I-b), (I-c), (I-d), (I-e), (I-f), (I-g), (I-h), (I-i), (I-j), or (I-k), the

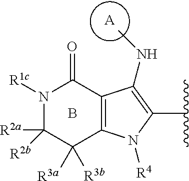

moiety is

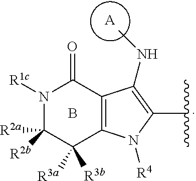

Non-Limiting Exemplary Compounds

In certain embodiments, the compound is selected from the group consisting of the compounds delineated in Table C1, or a pharmaceutically acceptable salt thereof.

TABLE C1
For certain compounds, the symbol * at a chiral
center denotes that this chiral center has
been resolved (i.e., is a single epimer) and the absolute
stereochemistry at that center has not been determined.

| No. | Structure |
|---|---|
| 101 | 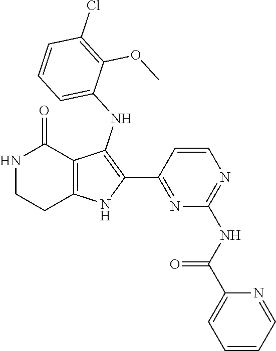 [SMILES: COc1c(Cl)cccc1Nc1c(-c2ccnc(NC(=O)c3ccccn3)n2)[nH]c2c1C(=O)NCC2] |
| 102 | 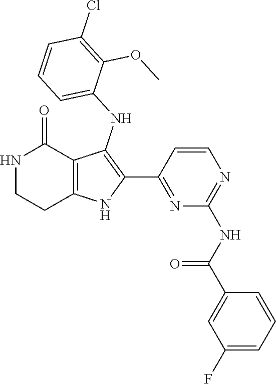 [SMILES: COc1c(Cl)cccc1Nc1c(-c2ccnc(NC(=O)c3cccc(F)c3)n2)[nH]c2c1C(=O)NCC2] |
| 103 | 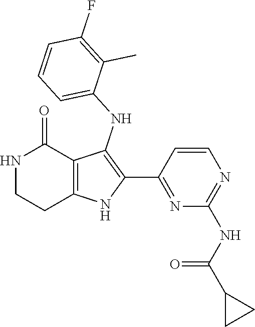 [SMILES: Cc1c(F)cccc1Nc1c(-c2ccnc(NC(=O)C3CC3)n2)[nH]c2c1C(=O)NCC2] |
| 319 | 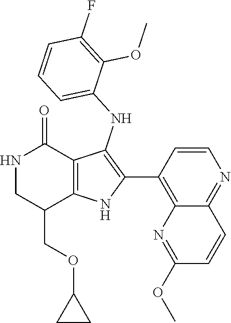 [SMILES: COc1ccc2nccc(-c3[nH]c4c(c3Nc3cccc(F)c3OC)C(=O)NCC4COC3CC3)c2n1] |
| 319a | 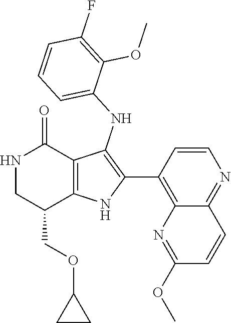 [SMILES: COc1ccc2nccc(-c3[nH]c4c(c3Nc3cccc(F)c3OC)C(=O)NC[C@H]4COC3CC3)c2n1] |
| 319b | 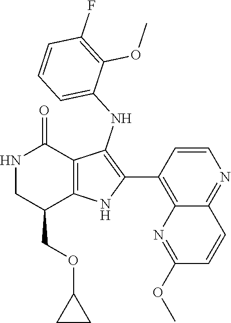 [SMILES: COc1ccc2nccc(-c3[nH]c4c(c3Nc3cccc(F)c3OC)C(=O)NC[C@@H]4COC3CC3)c2n1] |
| 104 | 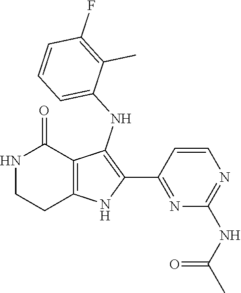 [SMILES: CC(=O)Nc1nccc(-c2[nH]c3c(c2Nc2cccc(F)c2C)C(=O)NCC3)n1] |
| 105 | 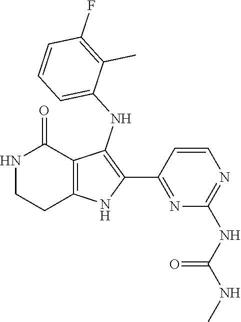 [SMILES: CNC(=O)Nc1nccc(-c2[nH]c3c(c2Nc2cccc(F)c2C)C(=O)NCC3)n1] |
| 106 | 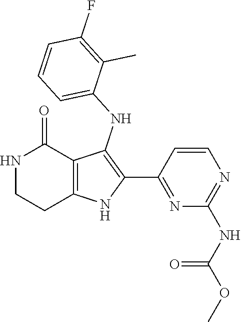 [SMILES: COC(=O)Nc1nccc(-c2[nH]c3c(c2Nc2cccc(F)c2C)C(=O)NCC3)n1] |
| 107 | 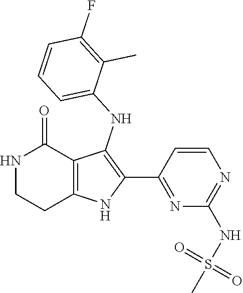 [SMILES: Cc1c(F)cccc1Nc1c(-c2ccnc(NS(C)(=O)=O)n2)[nH]c2c1C(=O)NCC2] |
| 320 | 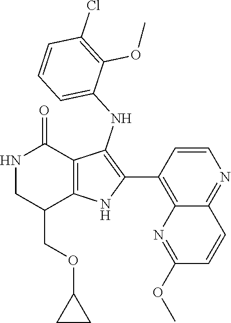 [SMILES: COc1ccc2nccc(-c3[nH]c4c(c3Nc3cccc(Cl)c3OC)C(=O)NCC4COC3CC3)c2n1] |
| 320a | 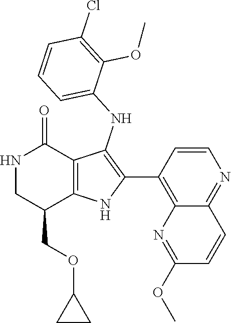 [SMILES: COc1ccc2nccc(-c3[nH]c4c(c3Nc3cccc(Cl)c3OC)C(=O)NC[C@@H]4COC3CC3)c2n1] |
| 320b | 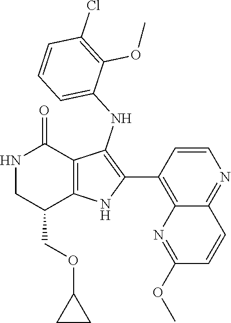 [SMILES: COc1ccc2nccc(-c3[nH]c4c(c3Nc3cccc(Cl)c3OC)C(=O)NC[C@H]4COC3CC3)c2n1] |
| 321 | 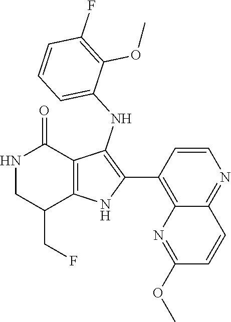 [SMILES: COc1ccc2nccc(-c3[nH]c4c(c3Nc3cccc(F)c3OC)C(=O)NCC4CF)c2n1] |
| 108 | 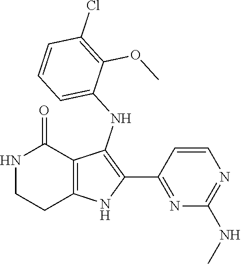 [SMILES: CNc1nccc(-c2[nH]c3c(c2Nc2cccc(Cl)c2OC)C(=O)NCC3)n1] |
| 109 | 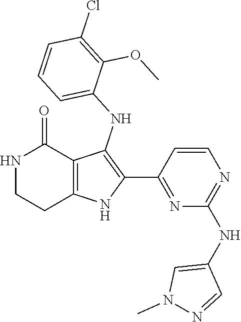 [SMILES: COc1c(Cl)cccc1Nc1c(-c2ccnc(Nc3cnn(C)c3)n2)[nH]c2c1C(=O)NCC2] |
| 110 | 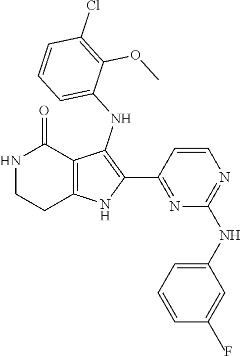 |
| 321a | 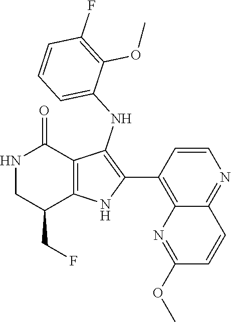 [SMILES: COc1ccc2nccc(-c3[nH]c4c(c3Nc3cccc(F)c3OC)C(=O)NC[C@@H]4CF)c2n1] |
| 321b | 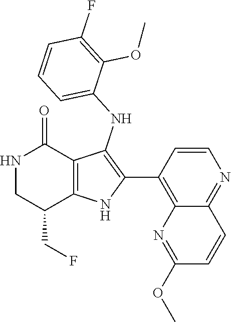 [SMILES: COc1ccc2nccc(-c3[nH]c4c(c3Nc3cccc(F)c3OC)C(=O)NC[C@H]4CF)c2n1] |
| 322 | 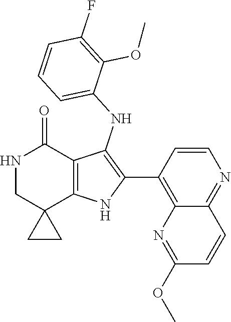 [SMILES: COc1ccc2nccc(-c3[nH]c4c(c3Nc3cccc(F)c3OC)C(=O)NCC43CC3)c2n1] |
| 111 | 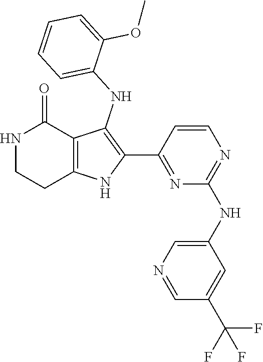 [SMILES: COc1ccccc1Nc1c(-c2ccnc(Nc3cncc(C(F)(F)F)c3)n2)[nH]c2c1C(=O)NCC2] |
| 112 | 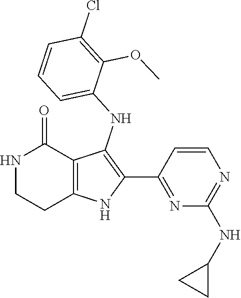 [SMILES: COc1c(Cl)cccc1Nc1c(-c2ccnc(NC3CC3)n2)[nH]c2c1C(=O)NCC2] |
| 113 | 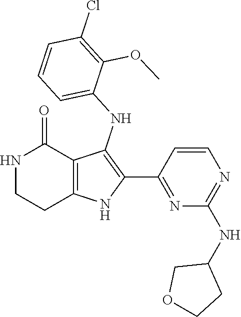 [SMILES: COc1c(Cl)cccc1Nc1c(-c2ccnc(NC3CCOC3)n2)[nH]c2c1C(=O)NCC2] |
| 323 | 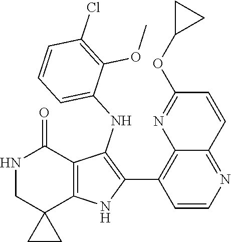 [SMILES: COc1c(Cl)cccc1Nc1c(-c2ccnc3ccc(OC4CC4)nc23)[nH]c2c1C(=O)NCC21CC1] |
| 324 | 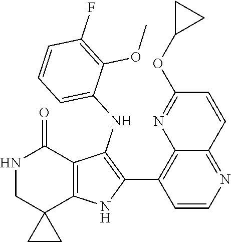 [SMILES: COc1c(F)cccc1Nc1c(-c2ccnc3ccc(OC4CC4)nc23)[nH]c2c1C(=O)NCC21CC1] |
| 325 | 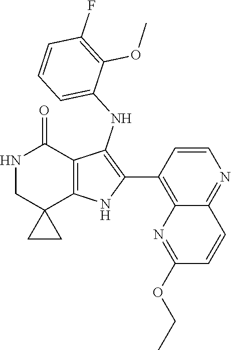 [SMILES: CCOc1ccc2nccc(-c3[nH]c4c(c3Nc3cccc(F)c3OC)C(=O)NCC43CC3)c2n1] |
| 113a | 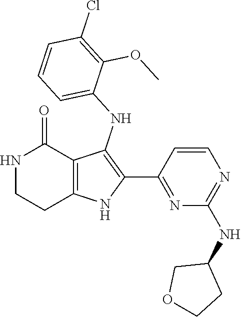 [SMILES: COc1c(Cl)cccc1Nc1c(-c2ccnc(N[C@H]3CCOC3)n2)[nH]c2c1C(=O)NCC2] |
| 113b | 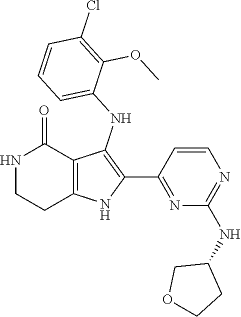 [SMILES: COc1c(Cl)cccc1Nc1c(-c2ccnc(N[C@@H]3CCOC3)n2)[nH]c2c1C(=O)NCC2] |
| 114 | 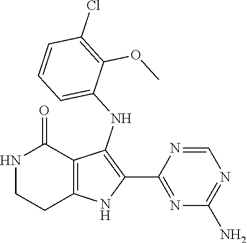 [SMILES: COc1c(Cl)cccc1Nc1c(-c2ncnc(N)n2)[nH]c2c1C(=O)NCC2] |
| 326 | 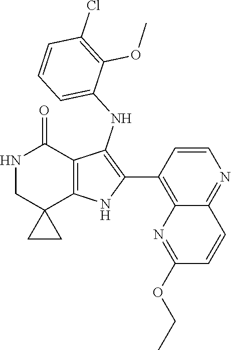 [SMILES: CCOc1ccc2nccc(-c3[nH]c4c(c3Nc3cccc(Cl)c3OC)C(=O)NCC43CC3)c2n1] |
| 327 | 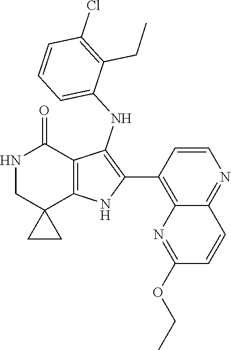 [SMILES: CCOc1ccc2nccc(-c3[nH]c4c(c3Nc3cccc(Cl)c3CC)C(=O)NCC43CC3)c2n1] |
| 328 | 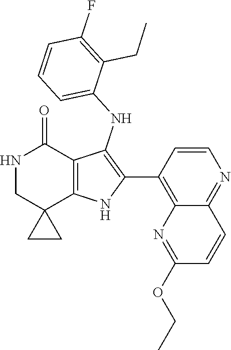 [SMILES: CCOc1ccc2nccc(-c3[nH]c4c(c3Nc3cccc(F)c3CC)C(=O)NCC43CC3)c2n1] |
| 115 | 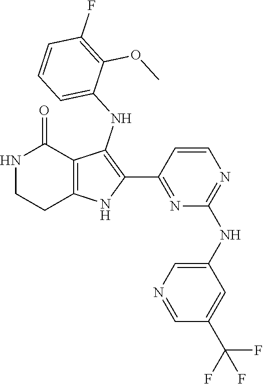 [SMILES: COc1c(F)cccc1Nc1c(-c2ccnc(Nc3cncc(C(F)(F)F)c3)n2)[nH]c2c1C(=O)NCC2] |
| 116 | 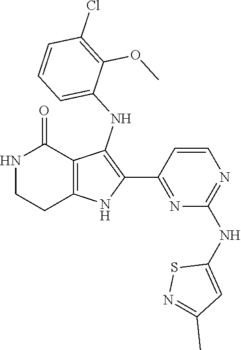 [SMILES: COc1c(Cl)cccc1Nc1c(-c2ccnc(Nc3cc(C)ns3)n2)[nH]c2c1C(=O)NCC2] |
| 117 | 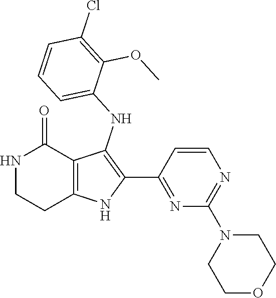 [SMILES: COc1c(Cl)cccc1Nc1c(-c2ccnc(N3CCOCC3)n2)[nH]c2c1C(=O)NCC2] |
| 329 | 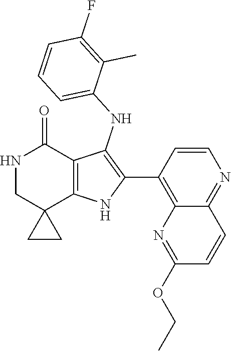 [SMILES: CCOc1ccc2nccc(-c3[nH]c4c(c3Nc3cccc(F)c3C)C(=O)NCC43CC3)c2n1] |
| 330 | 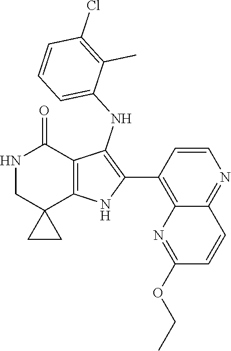 [SMILES: CCOc1ccc2nccc(-c3[nH]c4c(c3Nc3cccc(Cl)c3C)C(=O)NCC43CC3)c2n1] |
| 331 | 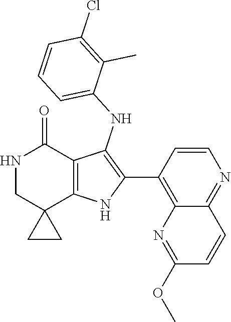 [SMILES: COc1ccc2nccc(-c3[nH]c4c(c3Nc3cccc(Cl)c3C)C(=O)NCC43CC3)c2n1] |
| 118 | 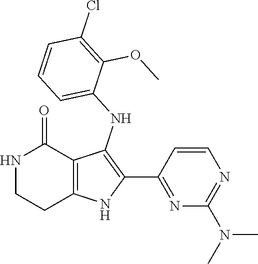 [SMILES: COc1c(Cl)cccc1Nc1c(-c2ccnc(N(C)C)n2)[nH]c2c1C(=O)NCC2] |
| 119 | 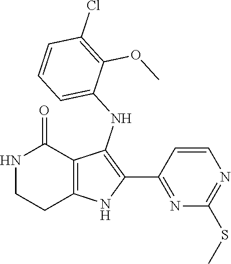 [SMILES: COc1c(Cl)cccc1Nc1c(-c2ccnc(SC)n2)[nH]c2c1C(=O)NCC2] |
| 120 | 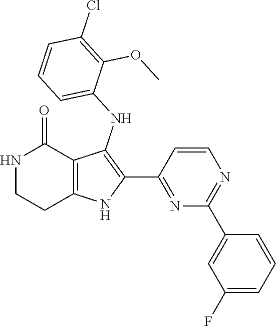 [SMILES: COc1c(Cl)cccc1Nc1c(-c2ccnc(-c3cccc(F)c3)n2)[nH]c2c1C(=O)NCC2] |
| 121 | 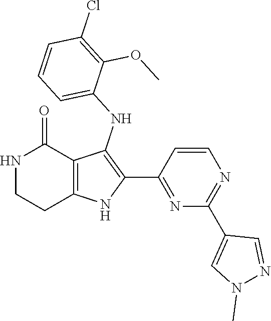 [SMILES: COc1c(Cl)cccc1Nc1c(-c2ccnc(-c3cnn(C)c3)n2)[nH]c2c1C(=O)NCC2] |
| 332 | 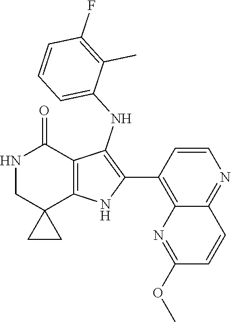 [SMILES: COc1ccc2nccc(-c3[nH]c4c(c3Nc3cccc(F)c3C)C(=O)NCC43CC3)c2n1] |
| 333 | 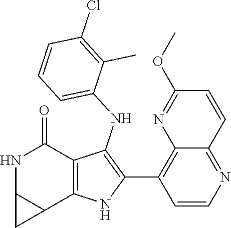 [SMILES: COc1ccc2nccc(-c3[nH]c4c(c3Nc3cccc(Cl)c3C)C(=O)NC3CC43)c2n1] |
| 333a | 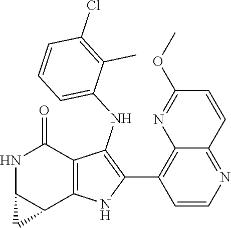 [SMILES: COc1ccc2nccc(-c3[nH]c4c(c3Nc3cccc(Cl)c3C)C(=O)N[C@H]3C[C@@H]43)c2n1] |
| 333b | 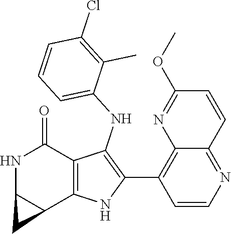 [SMILES: COc1ccc2nccc(-c3[nH]c4c(c3Nc3cccc(Cl)c3C)C(=O)N[C@@H]3C[C@H]43)c2n1] |
| 122 | 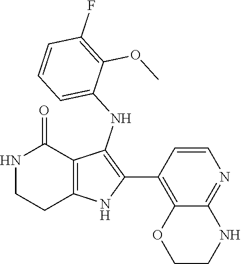 [SMILES: COc1c(F)cccc1Nc1c(-c2ccnc3c2OCCN3)[nH]c2c1C(=O)NCC2] |
| 123 | 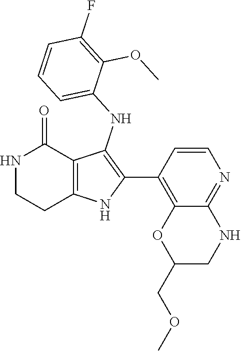 [SMILES: COCC1CNc2nccc(-c3[nH]c4c(c3Nc3cccc(F)c3OC)C(=O)NCC4)c2O1] |
| 123a | 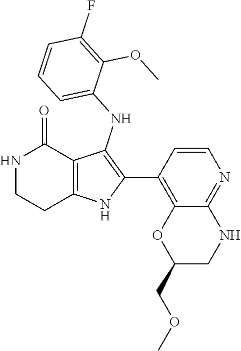 [SMILES: COC[C@@H]1CNc2nccc(-c3[nH]c4c(c3Nc3cccc(F)c3OC)C(=O)NCC4)c2O1] |
| 123b | 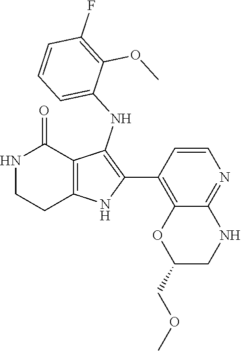 [SMILES: COC[C@H]1CNc2nccc(-c3[nH]c4c(c3Nc3cccc(F)c3OC)C(=O)NCC4)c2O1] |
| 334 | 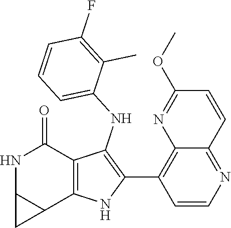 [SMILES: COc1ccc2nccc(-c3[nH]c4c(c3Nc3cccc(F)c3C)C(=O)NC3CC43)c2n1] |
| 334a | 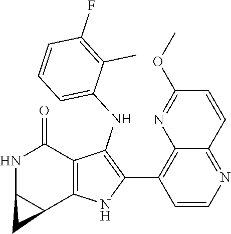 [SMILES: COc1ccc2nccc(-c3[nH]c4c(c3Nc3cccc(F)c3C)C(=O)N[C@@H]3C[C@H]43)c2n1] |
| 334b | 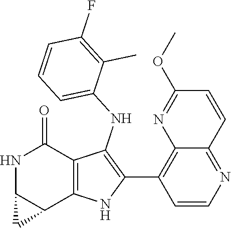 [SMILES: COc1ccc2nccc(-c3[nH]c4c(c3Nc3cccc(F)c3C)C(=O)N[C@H]3C[C@@H]43)c2n1] |
| 335 | 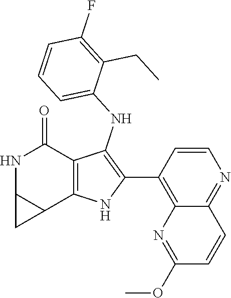 [SMILES: CCc1c(F)cccc1Nc1c(-c2ccnc3ccc(OC)nc23)[nH]c2c1C(=O)NC1CC21] |
| 124 | 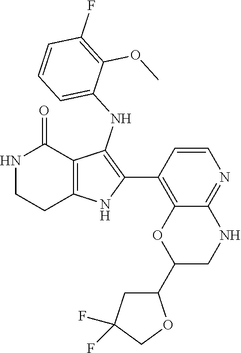 [SMILES: COc1c(F)cccc1Nc1c(-c2ccnc3c2OC(C2CC(F)(F)CO2)CN3)[nH]c2c1C(=O)NCC2] |
| 124a | 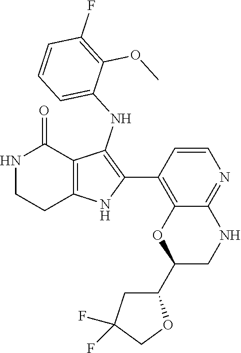 [SMILES: COc1c(F)cccc1Nc1c(-c2ccnc3c2O[C@H]([C@H]2CC(F)(F)CO2)CN3)[nH]c2c1C(=O)NCC2] |
| 124b | 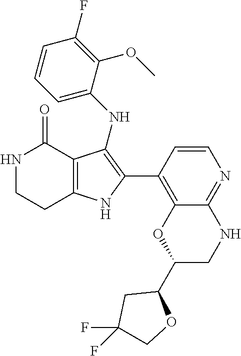 [SMILES: COc1c(F)cccc1Nc1c(-c2ccnc3c2O[C@@H]([C@@H]2CC(F)(F)CO2)CN3)[nH]c2c1C(=O)NCC2] |
| 335a | 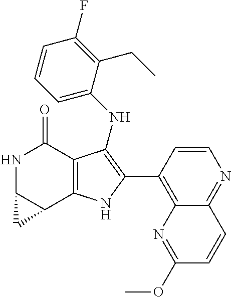 [SMILES: CCc1c(F)cccc1Nc1c(-c2ccnc3ccc(OC)nc23)[nH]c2c1C(=O)N[C@H]1C[C@@H]21] |
| 335b | 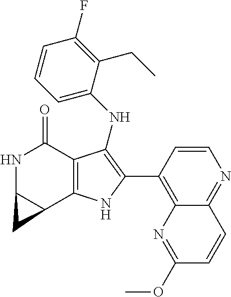 [SMILES: CCc1c(F)cccc1Nc1c(-c2ccnc3ccc(OC)nc23)[nH]c2c1C(=O)N[C@@H]1C[C@H]21] |
| 336 | 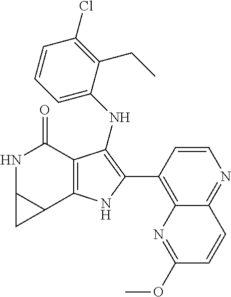 [SMILES: CCc1c(Cl)cccc1Nc1c(-c2ccnc3ccc(OC)nc23)[nH]c2c1C(=O)NC1CC21] |
| 124c | 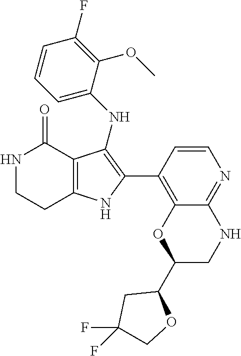 [SMILES: COc1c(F)cccc1Nc1c(-c2ccnc3c2O[C@H]([C@@H]2CC(F)(F)CO2)CN3)[nH]c2c1C(=O)NCC2] |
| 124d | 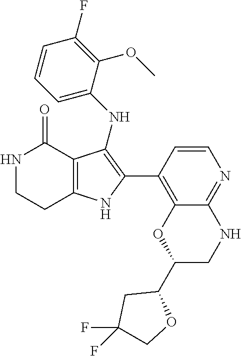 [SMILES: COc1c(F)cccc1Nc1c(-c2ccnc3c2O[C@@H]([C@H]2CC(F)(F)CO2)CN3)[nH]c2c1C(=O)NCC2] |
| 125 | 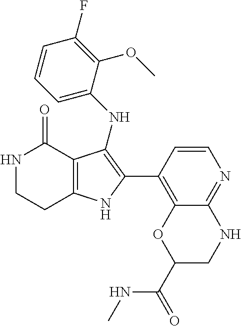 [SMILES: CNC(=O)C1CNc2nccc(-c3[nH]c4c(c3Nc3cccc(F)c3OC)C(=O)NCC4)c2O1] |
| 336a | 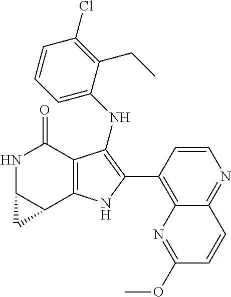 [SMILES: CCc1c(Cl)cccc1Nc1c(-c2ccnc3ccc(OC)nc23)[nH]c2c1C(=O)N[C@H]1C[C@@H]21] |
| 336b | 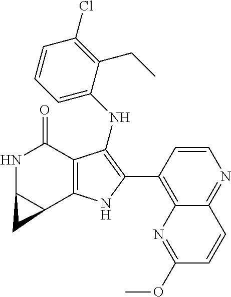 [SMILES: CCc1c(Cl)cccc1Nc1c(-c2ccnc3ccc(OC)nc23)[nH]c2c1C(=O)N[C@@H]1C[C@H]21] |
| 337 | 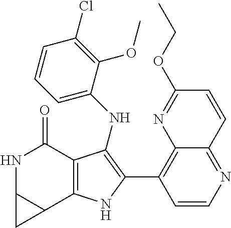 [SMILES: CCOc1ccc2nccc(-c3[nH]c4c(c3Nc3cccc(Cl)c3OC)C(=O)NC3CC43)c2n1] |
| 125a | 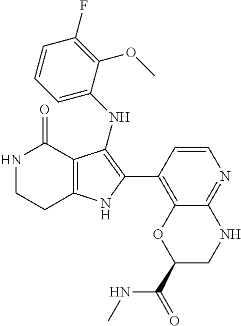 [SMILES: CNC(=O)[C@@H]1CNc2nccc(-c3[nH]c4c(c3Nc3cccc(F)c3OC)C(=O)NCC4)c2O1] |
| 125b | 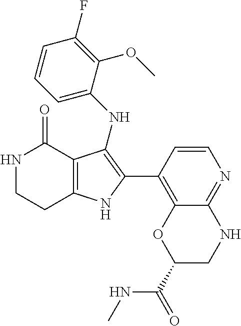 [SMILES: CNC(=O)[C@H]1CNc2nccc(-c3[nH]c4c(c3Nc3cccc(F)c3OC)C(=O)NCC4)c2O1] |
| 126 | 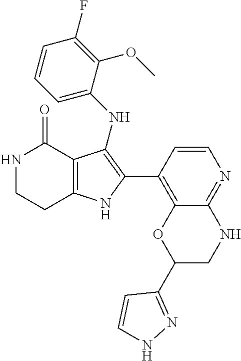 [SMILES: COc1c(F)cccc1Nc1c(-c2ccnc3c2OC(c2cc[nH]n2)CN3)[nH]c2c1C(=O)NCC2] |
| 337a | 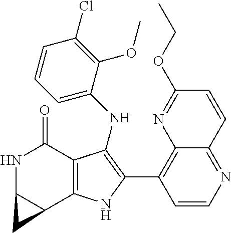 [SMILES: CCOc1ccc2nccc(-c3[nH]c4c(c3Nc3cccc(Cl)c3OC)C(=O)N[C@@H]3C[C@H]43)c2n1] |
| 337b | 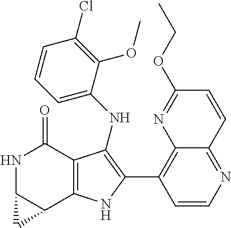 [SMILES: CCOc1ccc2nccc(-c3[nH]c4c(c3Nc3cccc(Cl)c3OC)C(=O)N[C@H]3C[C@@H]43)c2n1] |
| 338 | 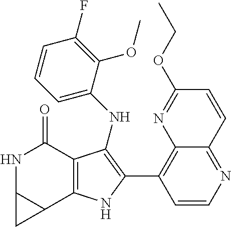 [SMILES: CCOc1ccc2nccc(-c3[nH]c4c(c3Nc3cccc(F)c3OC)C(=O)NC3CC43)c2n1] |
| 126a | 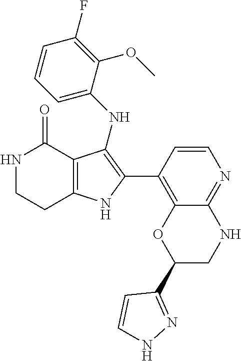 [SMILES: COc1c(F)cccc1Nc1c(-c2ccnc3c2O[C@H](c2cc[nH]n2)CN3)[nH]c2c1C(=O)NCC2] |
| 126b | 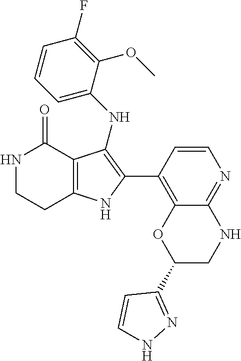 [SMILES: COc1c(F)cccc1Nc1c(-c2ccnc3c2O[C@@H](c2cc[nH]n2)CN3)[nH]c2c1C(=O)NCC2] |
| 127 | 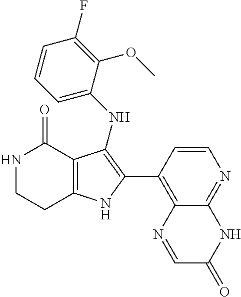 [SMILES: COc1c(F)cccc1Nc1c(-c2ccnc3[nH]c(=O)cnc23)[nH]c2c1C(=O)NCC2] |
| 338a | 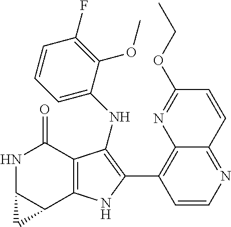 [SMILES: CCOc1ccc2nccc(-c3[nH]c4c(c3Nc3cccc(F)c3OC)C(=O)N[C@H]3C[C@@H]43)c2n1] |
| 338b | 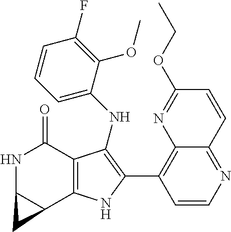 [SMILES: CCOc1ccc2nccc(-c3[nH]c4c(c3Nc3cccc(F)c3OC)C(=O)N[C@@H]3C[C@H]43)c2n1] |
| 339 | 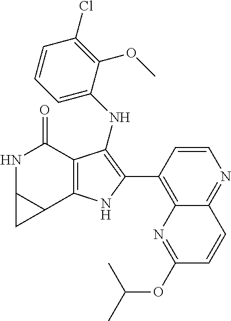 [SMILES: COc1c(Cl)cccc1Nc1c(-c2ccnc3ccc(OC(C)C)nc23)[nH]c2c1C(=O)NC1CC21] |
| 128 | 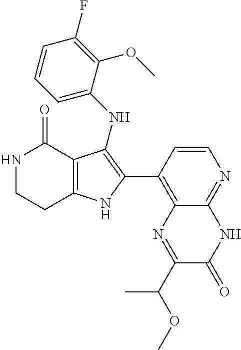 [SMILES: COc1c(F)cccc1Nc1c(-c2ccnc3[nH]c(=O)c(C(C)OC)nc23)[nH]c2c1C(=O)NCC2] |
| 128a | 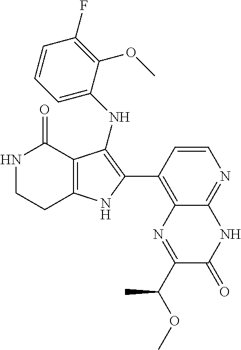 [SMILES: COc1c(F)cccc1Nc1c(-c2ccnc3[nH]c(=O)c([C@H](C)OC)nc23)[nH]c2c1C(=O)NCC2] |
| 128b | 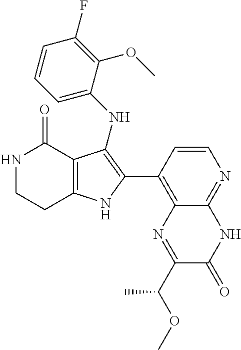 [SMILES: COc1c(F)cccc1Nc1c(-c2ccnc3[nH]c(=O)c([C@@H](C)OC)nc23)[nH]c2c1C(=O)NCC2] |
| 339a | 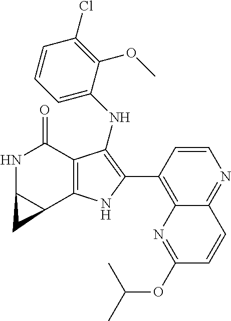 [SMILES: COc1c(Cl)cccc1Nc1c(-c2ccnc3ccc(OC(C)C)nc23)[nH]c2c1C(=O)N[C@@H]1C[C@H]21] |
| 339b | 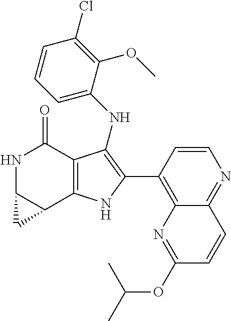 [SMILES: COc1c(Cl)cccc1Nc1c(-c2ccnc3ccc(OC(C)C)nc23)[nH]c2c1C(=O)N[C@H]1C[C@@H]21] |
| 340 | 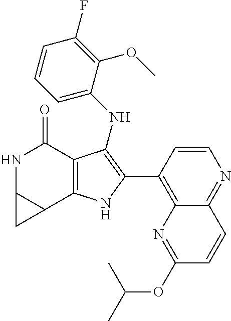 [SMILES: COc1c(F)cccc1Nc1c(-c2ccnc3ccc(OC(C)C)nc23)[nH]c2c1C(=O)NC1CC21] |
| 129 | 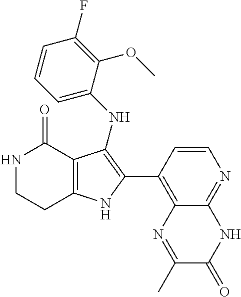 [SMILES: COc1c(F)cccc1Nc1c(-c2ccnc3[nH]c(=O)c(C)nc23)[nH]c2c1C(=O)NCC2] |
| 130 | 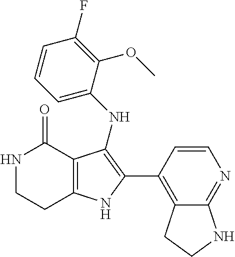 [SMILES: COc1c(F)cccc1Nc1c(-c2ccnc3c2CCN3)[nH]c2c1C(=O)NCC2] |
| 131 | 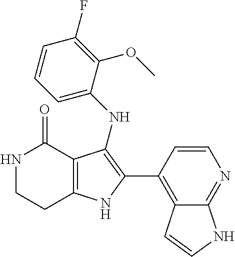 [SMILES: COc1c(F)cccc1Nc1c(-c2ccnc3[nH]ccc23)[nH]c2c1C(=O)NCC2] |
| 132 | 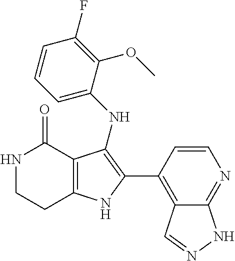 [SMILES: COc1c(F)cccc1Nc1c(-c2ccnc3[nH]ncc23)[nH]c2c1C(=O)NCC2] |
| 340a | 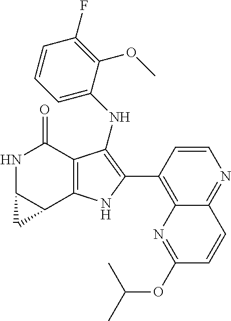 [SMILES: COc1c(F)cccc1Nc1c(-c2ccnc3ccc(OC(C)C)nc23)[nH]c2c1C(=O)N[C@H]1C[C@@H]21] |
| 340b | 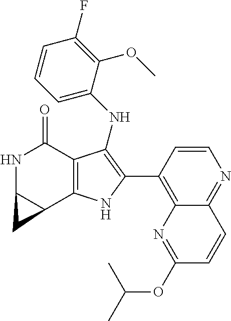 [SMILES: COc1c(F)cccc1Nc1c(-c2ccnc3ccc(OC(C)C)nc23)[nH]c2c1C(=O)N[C@@H]1C[C@H]21] |
| 341 | 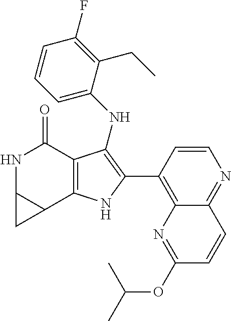 [SMILES: CCc1c(F)cccc1Nc1c(-c2ccnc3ccc(OC(C)C)nc23)[nH]c2c1C(=O)NC1CC21] |
| 341a | 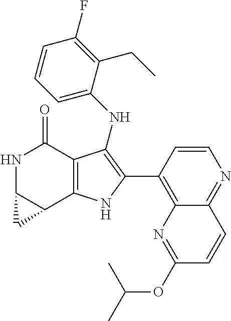 [SMILES: CCc1c(F)cccc1Nc1c(-c2ccnc3ccc(OC(C)C)nc23)[nH]c2c1C(=O)N[C@H]1C[C@@H]21] |
| 133 | 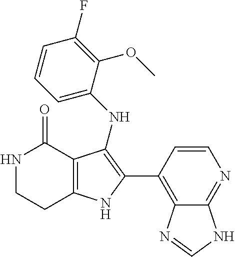 [SMILES: COc1c(F)cccc1Nc1c(-c2ccnc3[nH]cnc23)[nH]c2c1C(=O)NCC2] |
| 134 | 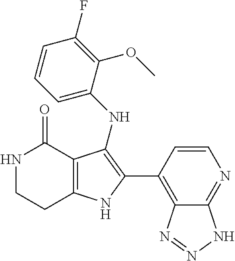 [SMILES: COc1c(F)cccc1Nc1c(-c2ccnc3[nH]nnc23)[nH]c2c1C(=O)NCC2] |
| 135 | 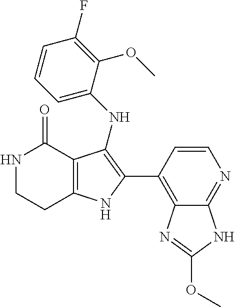 [SMILES: COc1nc2c(-c3[nH]c4c(c3Nc3cccc(F)c3OC)C(=O)NCC4)ccnc2[nH]1] |
| 136 | 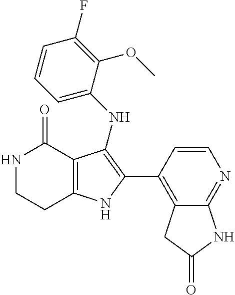 [SMILES: COc1c(F)cccc1Nc1c(-c2ccnc3c2CC(=O)N3)[nH]c2c1C(=O)NCC2] |
| 341b | 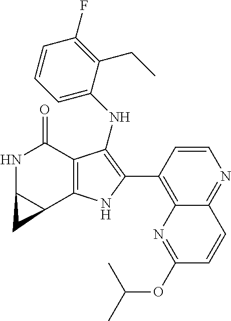 [SMILES: CCc1c(F)cccc1Nc1c(-c2ccnc3ccc(OC(C)C)nc23)[nH]c2c1C(=O)N[C@@H]1C[C@H]21] |
| 342 | 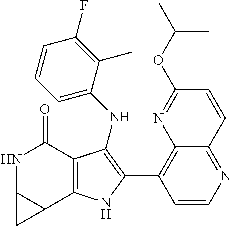 [SMILES: Cc1c(F)cccc1Nc1c(-c2ccnc3ccc(OC(C)C)nc23)[nH]c2c1C(=O)NC1CC21] |
| 342a | 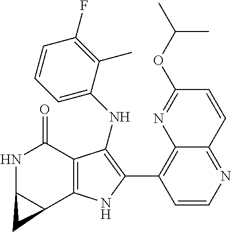 [SMILES: Cc1c(F)cccc1Nc1c(-c2ccnc3ccc(OC(C)C)nc23)[nH]c2c1C(=O)N[C@@H]1C[C@H]21] |
| 342b | 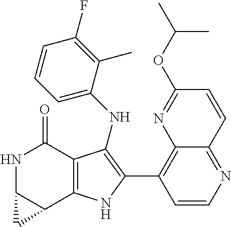 [SMILES: Cc1c(F)cccc1Nc1c(-c2ccnc3ccc(OC(C)C)nc23)[nH]c2c1C(=O)N[C@H]1C[C@@H]21] |
| 137 | 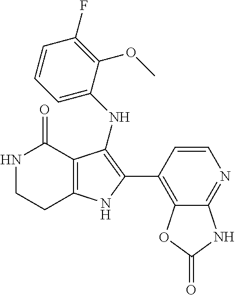 [SMILES: COc1c(F)cccc1Nc1c(-c2ccnc3[nH]c(=O)oc23)[nH]c2c1C(=O)NCC2] |
| 138 | 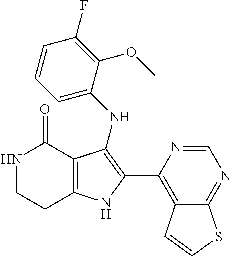 [SMILES: COc1c(F)cccc1Nc1c(-c2ncnc3sccc23)[nH]c2c1C(=O)NCC2] |
| 139 | 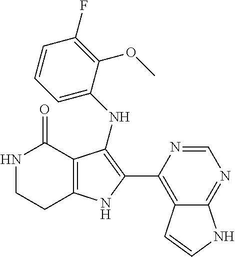 [SMILES: COc1c(F)cccc1Nc1c(-c2ncnc3[nH]ccc23)[nH]c2c1C(=O)NCC2] |
| 140 | 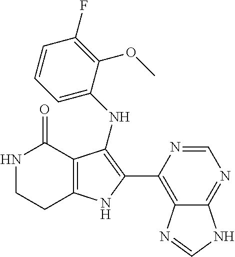 [SMILES: COc1c(F)cccc1Nc1c(-c2ncnc3[nH]cnc23)[nH]c2c1C(=O)NCC2] |
| 343 | 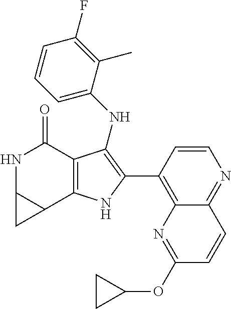 [SMILES: Cc1c(F)cccc1Nc1c(-c2ccnc3ccc(OC4CC4)nc23)[nH]c2c1C(=O)NC1CC21] |
| 343a | 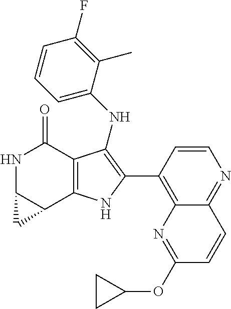 [SMILES: Cc1c(F)cccc1Nc1c(-c2ccnc3ccc(OC4CC4)nc23)[nH]c2c1C(=O)N[C@H]1C[C@@H]21] |
| 343b | 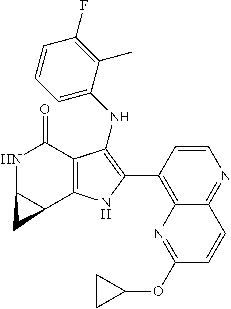 [SMILES: Cc1c(F)cccc1Nc1c(-c2ccnc3ccc(OC4CC4)nc23)[nH]c2c1C(=O)N[C@@H]1C[C@H]21] |
| 344 | 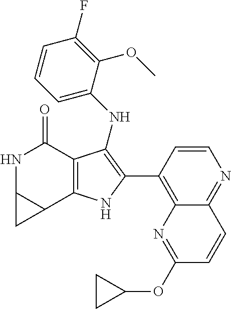 [SMILES: COc1c(F)cccc1Nc1c(-c2ccnc3ccc(OC4CC4)nc23)[nH]c2c1C(=O)NC1CC21] |
| 141 | 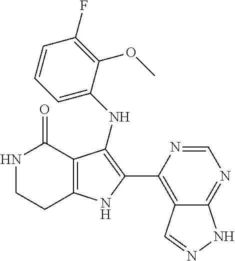 [SMILES: COc1c(F)cccc1Nc1c(-c2ncnc3[nH]ncc23)[nH]c2c1C(=O)NCC2] |
| 142 | 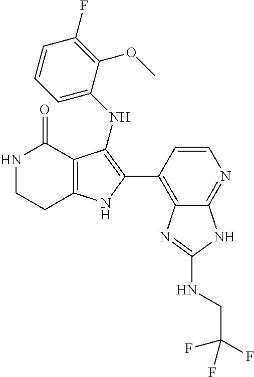 [SMILES: COc1c(F)cccc1Nc1c(-c2ccnc3[nH]c(NCC(F)(F)F)nc23)[nH]c2c1C(=O)NCC2] |
| 143 | 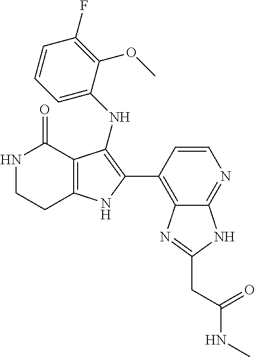 [SMILES: CNC(=O)Cc1nc2c(-c3[nH]c4c(c3Nc3cccc(F)c3OC)C(=O)NCC4)ccnc2[nH]1] |
| 344a | 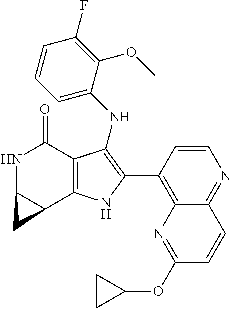 [SMILES: COc1c(F)cccc1Nc1c(-c2ccnc3ccc(OC4CC4)nc23)[nH]c2c1C(=O)N[C@@H]1C[C@H]21] |
| 344b | 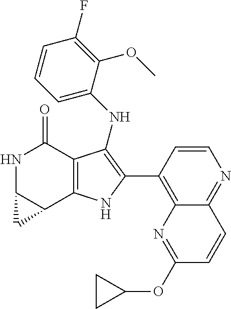 [SMILES: COc1c(F)cccc1Nc1c(-c2ccnc3ccc(OC4CC4)nc23)[nH]c2c1C(=O)N[C@H]1C[C@@H]21] |
| 345 | 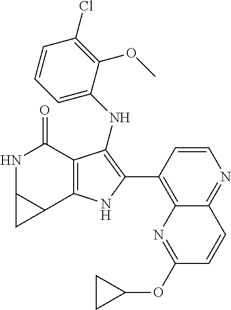 [SMILES: COc1c(Cl)cccc1Nc1c(-c2ccnc3ccc(OC4CC4)nc23)[nH]c2c1C(=O)NC1CC21] |
| 144 | 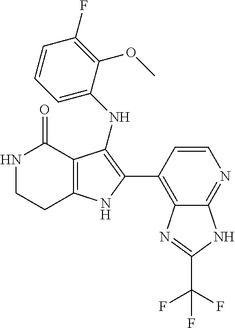 [SMILES: COc1c(F)cccc1Nc1c(-c2ccnc3[nH]c(C(F)(F)F)nc23)[nH]c2c1C(=O)NCC2] |
| 145 | 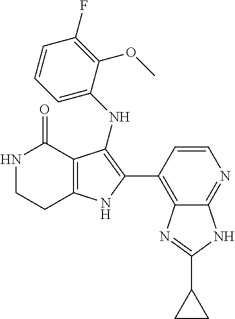 [SMILES: COc1c(F)cccc1Nc1c(-c2ccnc3[nH]c(C4CC4)nc23)[nH]c2c1C(=O)NCC2] |
| 146 | 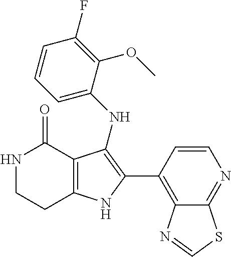 [SMILES: COc1c(F)cccc1Nc1c(-c2ccnc3scnc23)[nH]c2c1C(=O)NCC2] |
| 147 | 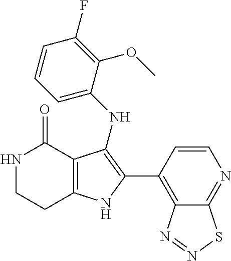 [SMILES: COc1c(F)cccc1Nc1c(-c2ccnc3snnc23)[nH]c2c1C(=O)NCC2] |
| 345a | 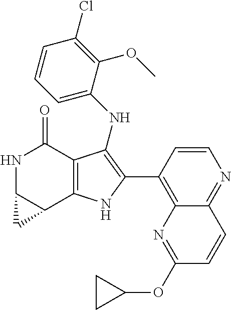 [SMILES: COc1c(Cl)cccc1Nc1c(-c2ccnc3ccc(OC4CC4)nc23)[nH]c2c1C(=O)N[C@H]1C[C@@H]21] |
| 345b | 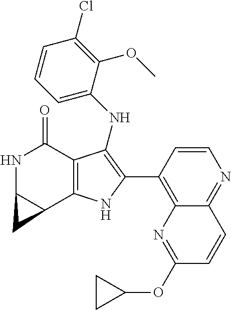 [SMILES: COc1c(Cl)cccc1Nc1c(-c2ccnc3ccc(OC4CC4)nc23)[nH]c2c1C(=O)N[C@@H]1C[C@H]21] |
| 346 | 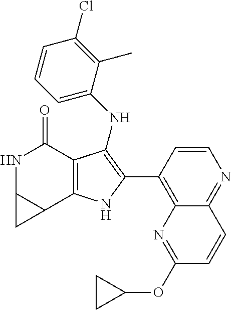 [SMILES: Cc1c(Cl)cccc1Nc1c(-c2ccnc3ccc(OC4CC4)nc23)[nH]c2c1C(=O)NC1CC21] |
| 346a | 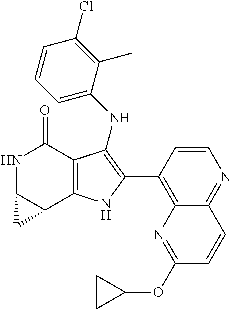 [SMILES: Cc1c(Cl)cccc1Nc1c(-c2ccnc3ccc(OC4CC4)nc23)[nH]c2c1C(=O)N[C@H]1C[C@@H]21] |
| 148 | 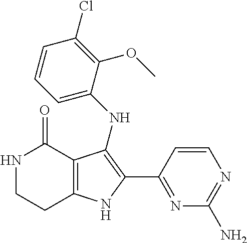 [SMILES: COc1c(Cl)cccc1Nc1c(-c2ccnc(N)n2)[nH]c2c1C(=O)NCC2] |
| 149 | 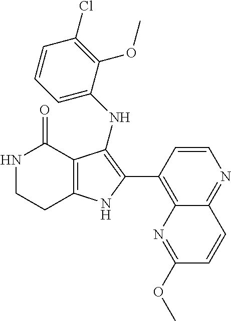 [SMILES: COc1ccc2nccc(-c3[nH]c4c(c3Nc3cccc(Cl)c3OC)C(=O)NCC4)c2n1] |
| 150 | 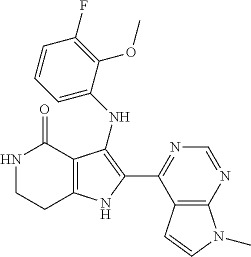 [SMILES: COc1c(F)cccc1Nc1c(-c2ncnc3c2ccn3C)[nH]c2c1C(=O)NCC2] |
| 151 | 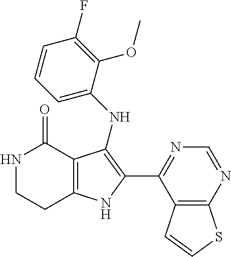 [SMILES: COc1c(F)cccc1Nc1c(-c2ncnc3sccc23)[nH]c2c1C(=O)NCC2] |
| 346b | 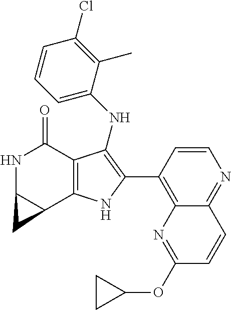 [SMILES: Cc1c(Cl)cccc1Nc1c(-c2ccnc3ccc(OC4CC4)nc23)[nH]c2c1C(=O)N[C@@H]1C[C@H]21] |
| 347 | 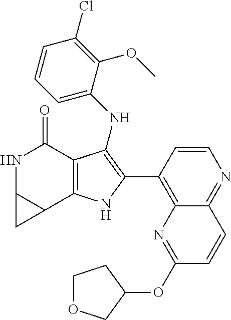 [SMILES: COc1c(Cl)cccc1Nc1c(-c2ccnc3ccc(OC4CCOC4)nc23)[nH]c2c1C(=O)NC1CC21] |
| 347a | 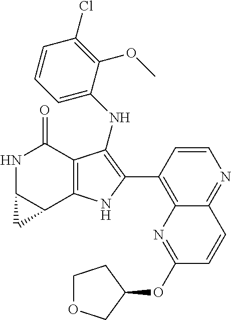 [SMILES: COc1c(Cl)cccc1Nc1c(-c2ccnc3ccc(O[C@@H]4CCOC4)nc23)[nH]c2c1C(=O)N[C@H]1C[C@@H]21] |
| 347b | 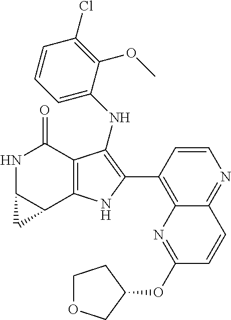 [SMILES: COc1c(Cl)cccc1Nc1c(-c2ccnc3ccc(O[C@H]4CCOC4)nc23)[nH]c2c1C(=O)N[C@H]1C[C@@H]21] |
| 152 | 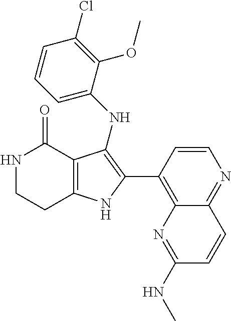 [SMILES: CNc1ccc2nccc(-c3[nH]c4c(c3Nc3cccc(Cl)c3OC)C(=O)NCC4)c2n1] |
| 153 | 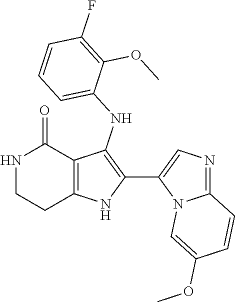 [SMILES: COc1ccc2ncc(-c3[nH]c4c(c3Nc3cccc(F)c3OC)C(=O)NCC4)n2c1] |
| 154 | 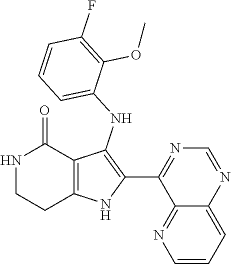 [SMILES: COc1c(F)cccc1Nc1c(-c2ncnc3cccnc23)[nH]c2c1C(=O)NCC2] |
| 155 | 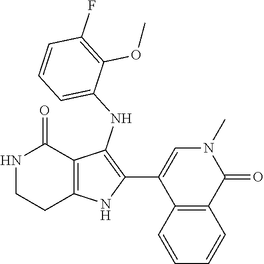 [SMILES: COc1c(F)cccc1Nc1c(-c2cn(C)c(=O)c3ccccc23)[nH]c2c1C(=O)NCC2] |
| 347c | 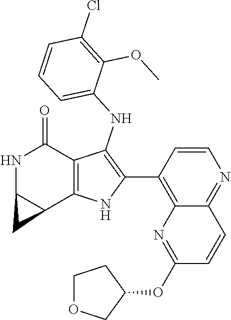 [SMILES: COc1c(Cl)cccc1Nc1c(-c2ccnc3ccc(O[C@H]4CCOC4)nc23)[nH]c2c1C(=O)N[C@@H]1C[C@H]21] |
| 347d | 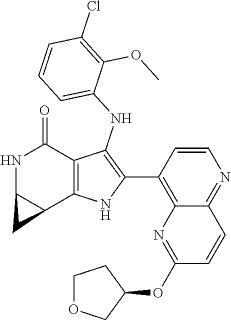 [SMILES: COc1c(Cl)cccc1Nc1c(-c2ccnc3ccc(O[C@@H]4CCOC4)nc23)[nH]c2c1C(=O)N[C@@H]1C[C@H]21] |
| 348 | 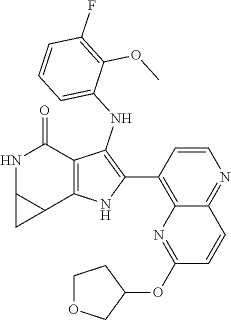 [SMILES: COc1c(F)cccc1Nc1c(-c2ccnc3ccc(OC4CCOC4)nc23)[nH]c2c1C(=O)NC1CC21] |
| 348a | 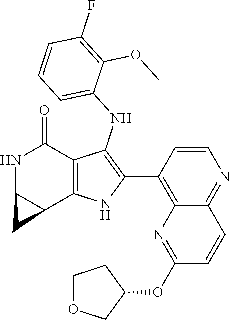 [SMILES: COc1c(F)cccc1Nc1c(-c2ccnc3ccc(O[C@H]4CCOC4)nc23)[nH]c2c1C(=O)N[C@@H]1C[C@H]21] |
| 156 | 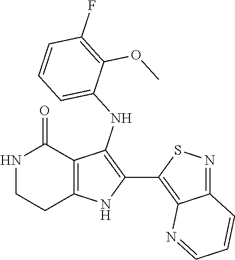 [SMILES: COc1c(F)cccc1Nc1c(-c2snc3cccnc23)[nH]c2c1C(=O)NCC2] |
| 157 | 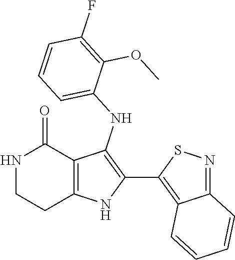 [SMILES: COc1c(F)cccc1Nc1c(-c2snc3ccccc23)[nH]c2c1C(=O)NCC2] |
| 158 | 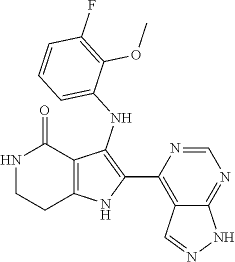 [SMILES: COc1c(F)cccc1Nc1c(-c2ncnc3[nH]ncc23)[nH]c2c1C(=O)NCC2] |
| 159 | 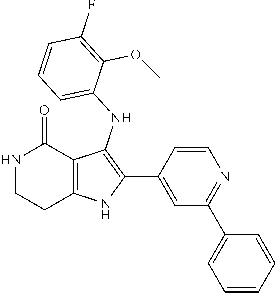 [SMILES: COc1c(F)cccc1Nc1c(-c2ccnc(-c3ccccc3)c2)[nH]c2c1C(=O)NCC2] |
| 348b | 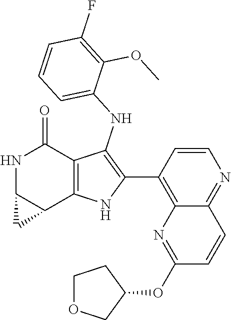 [SMILES: COc1c(F)cccc1Nc1c(-c2ccnc3ccc(O[C@H]4CCOC4)nc23)[nH]c2c1C(=O)N[C@H]1C[C@@H]21] |
| 348c | 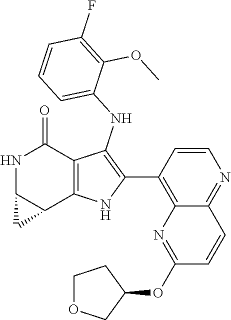 [SMILES: COc1c(F)cccc1Nc1c(-c2ccnc3ccc(O[C@@H]4CCOC4)nc23)[nH]c2c1C(=O)N[C@H]1C[C@@H]21] |
| 348d | 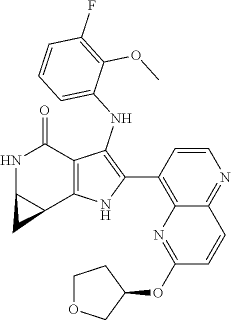 [SMILES: COc1c(F)cccc1Nc1c(-c2ccnc3ccc(O[C@@H]4CCOC4)nc23)[nH]c2c1C(=O)N[C@@H]1C[C@H]21] |
| 349 | 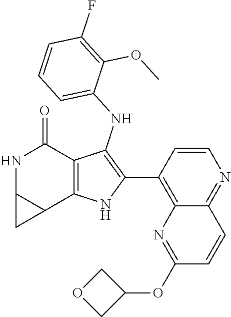 [SMILES: COc1c(F)cccc1Nc1c(-c2ccnc3ccc(OC4COC4)nc23)[nH]c2c1C(=O)NC1CC21] |
| 160 | 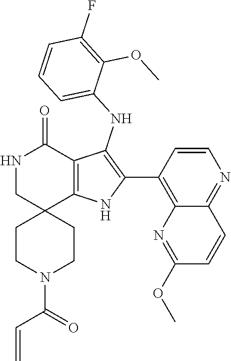 [SMILES: C=CC(=O)N1CCC2(CC1)CNC(=O)c1c2[nH]c(-c2ccnc3ccc(OC)nc23)c1Nc1cccc(F)c1OC] |
| 161 | 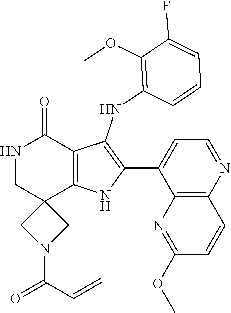 [SMILES: C=CC(=O)N1CC2(CNC(=O)c3c2[nH]c(-c2ccnc4ccc(OC)nc24)c3Nc2cccc(F)c2OC)C1] |
| 162 | 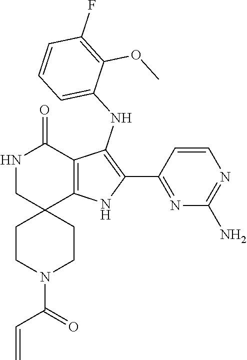 [SMILES: C=CC(=O)N1CCC2(CC1)CNC(=O)c1c2[nH]c(-c2ccnc(N)n2)c1Nc1cccc(F)c1OC] |
| 349a | 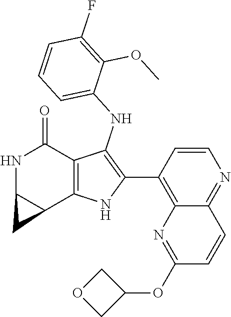 [SMILES: COc1c(F)cccc1Nc1c(-c2ccnc3ccc(OC4COC4)nc23)[nH]c2c1C(=O)N[C@@H]1C[C@H]21] |
| 349b | 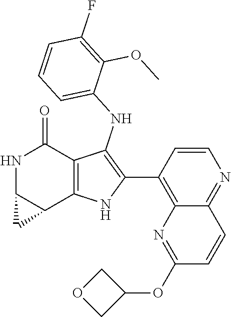 [SMILES: COc1c(F)cccc1Nc1c(-c2ccnc3ccc(OC4COC4)nc23)[nH]c2c1C(=O)N[C@H]1C[C@@H]21] |
| 350 | 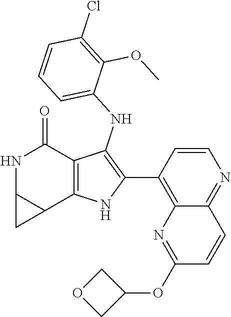 [SMILES: COc1c(Cl)cccc1Nc1c(-c2ccnc3ccc(OC4COC4)nc23)[nH]c2c1C(=O)NC1CC21] |
| 163 | 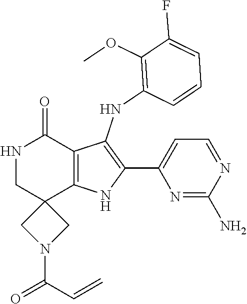 [SMILES: C=CC(=O)N1CC2(CNC(=O)c3c2[nH]c(-c2ccnc(N)n2)c3Nc2cccc(F)c2OC)C1] |
| 164 | 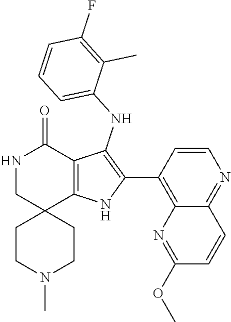 [SMILES: COc1ccc2nccc(-c3[nH]c4c(c3Nc3cccc(F)c3C)C(=O)NCC43CCN(C)CC3)c2n1] |
| 165 | 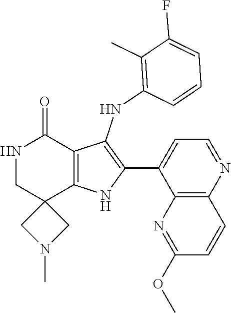 [SMILES: COc1ccc2nccc(-c3[nH]c4c(c3Nc3cccc(F)c3C)C(=O)NCC43CN(C)C3)c2n1] |
| 166 | 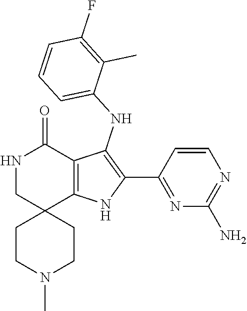 [SMILES: Cc1c(F)cccc1Nc1c(-c2ccnc(N)n2)[nH]c2c1C(=O)NCC21CCN(C)CC1] |
| 350a | 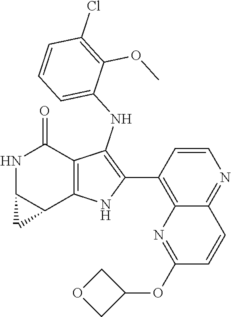 [SMILES: COc1c(Cl)cccc1Nc1c(-c2ccnc3ccc(OC4COC4)nc23)[nH]c2c1C(=O)N[C@H]1C[C@@H]21] |
| 350b | 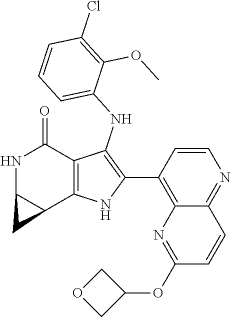 [SMILES: COc1c(Cl)cccc1Nc1c(-c2ccnc3ccc(OC4COC4)nc23)[nH]c2c1C(=O)N[C@@H]1C[C@H]21] |
| 351 | 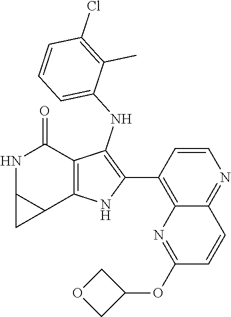 [SMILES: Cc1c(Cl)cccc1Nc1c(-c2ccnc3ccc(OC4COC4)nc23)[nH]c2c1C(=O)NC1CC21] |
| 351a | 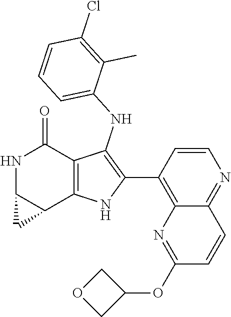 [SMILES: Cc1c(Cl)cccc1Nc1c(-c2ccnc3ccc(OC4COC4)nc23)[nH]c2c1C(=O)N[C@H]1C[C@@H]21] |
| 167 | 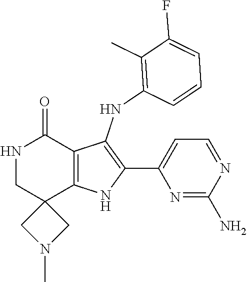 [SMILES: Cc1c(F)cccc1Nc1c(-c2ccnc(N)n2)[nH]c2c1C(=O)NCC21CN(C)C1] |
| 168 | 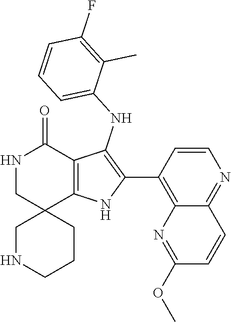 [SMILES: COc1ccc2nccc(-c3[nH]c4c(c3Nc3cccc(F)c3C)C(=O)NCC43CCCNC3)c2n1] |
| 168a | 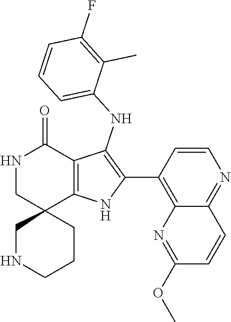 [SMILES: COc1ccc2nccc(-c3[nH]c4c(c3Nc3cccc(F)c3C)C(=O)NC[C@@]43CCCNC3)c2n1] |
| 168b | 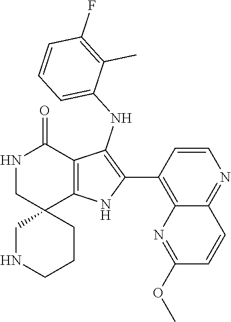 [SMILES: COc1ccc2nccc(-c3[nH]c4c(c3Nc3cccc(F)c3C)C(=O)NC[C@]43CCCNC3)c2n1] |
| 351b | 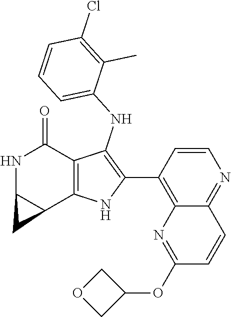 [SMILES: Cc1c(Cl)cccc1Nc1c(-c2ccnc3ccc(OC4COC4)nc23)[nH]c2c1C(=O)N[C@@H]1C[C@H]21] |
| 352 | 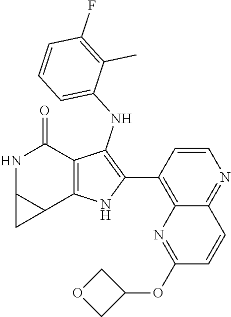 [SMILES: Cc1c(F)cccc1Nc1c(-c2ccnc3ccc(OC4COC4)nc23)[nH]c2c1C(=O)NC1CC21] |
| 352a | 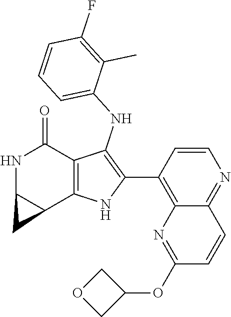 [SMILES: Cc1c(F)cccc1Nc1c(-c2ccnc3ccc(OC4COC4)nc23)[nH]c2c1C(=O)N[C@@H]1C[C@H]21] |
| 352b | 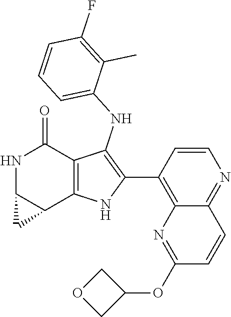 [SMILES: Cc1c(F)cccc1Nc1c(-c2ccnc3ccc(OC4COC4)nc23)[nH]c2c1C(=O)N[C@H]1C[C@@H]21] |
| 169 | 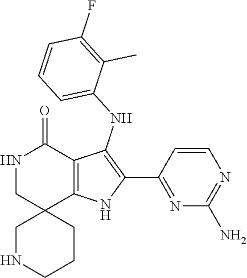 [SMILES: Cc1c(F)cccc1Nc1c(-c2ccnc(N)n2)[nH]c2c1C(=O)NCC21CCCNC1] |
| 169a | 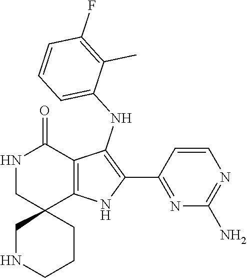 [SMILES: Cc1c(F)cccc1Nc1c(-c2ccnc(N)n2)[nH]c2c1C(=O)NC[C@@]21CCCNC1] |
| 169b | 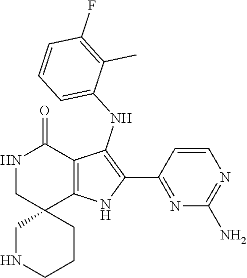 [SMILES: Cc1c(F)cccc1Nc1c(-c2ccnc(N)n2)[nH]c2c1C(=O)NC[C@]21CCCNC1] |
| 353 | 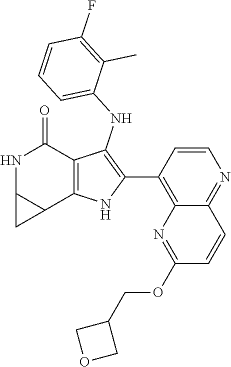 [SMILES: Cc1c(F)cccc1Nc1c(-c2ccnc3ccc(OCC4COC4)nc23)[nH]c2c1C(=O)NC1CC21] |
| 353a | 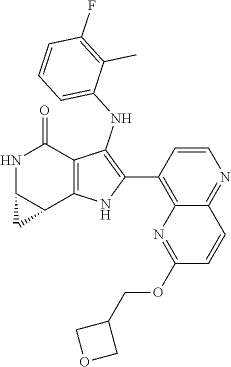 [SMILES: Cc1c(F)cccc1Nc1c(-c2ccnc3ccc(OCC4COC4)nc23)[nH]c2c1C(=O)N[C@H]1C[C@@H]21] |
| 353b | 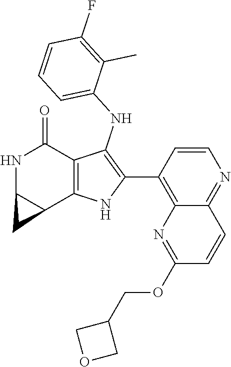 [SMILES: Cc1c(F)cccc1Nc1c(-c2ccnc3ccc(OCC4COC4)nc23)[nH]c2c1C(=O)N[C@@H]1C[C@H]21] |
| 170 | 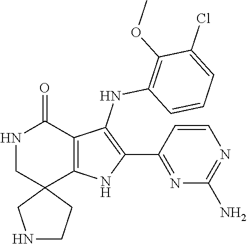 [SMILES: COc1c(Cl)cccc1Nc1c(-c2ccnc(N)n2)[nH]c2c1C(=O)NCC21CCNC1] |
| 170a | 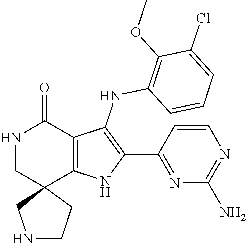 [SMILES: COc1c(Cl)cccc1Nc1c(-c2ccnc(N)n2)[nH]c2c1C(=O)NC[C@@]21CCNC1] |
| 170b | 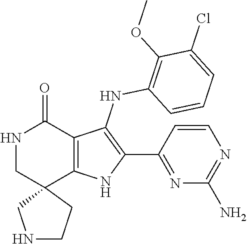 [SMILES: COc1c(Cl)cccc1Nc1c(-c2ccnc(N)n2)[nH]c2c1C(=O)NC[C@]21CCNC1] |
| 354 | 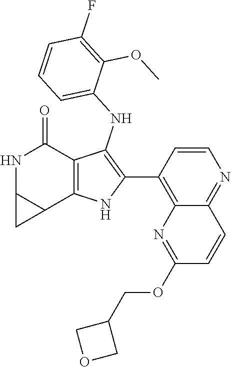 [SMILES: COc1c(F)cccc1Nc1c(-c2ccnc3ccc(OCC4COC4)nc23)[nH]c2c1C(=O)NC1CC21] |
| 354a | 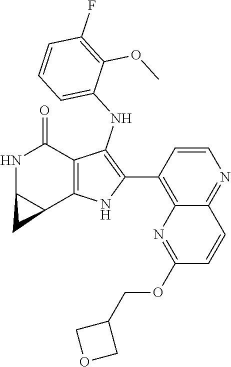 [SMILES: COc1c(F)cccc1Nc1c(-c2ccnc3ccc(OCC4COC4)nc23)[nH]c2c1C(=O)N[C@@H]1C[C@H]21] |
| 354b | 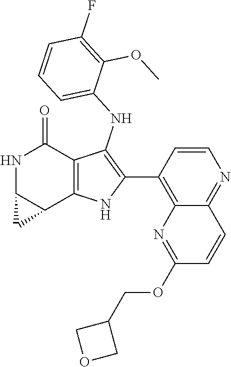 [SMILES: COc1c(F)cccc1Nc1c(-c2ccnc3ccc(OCC4COC4)nc23)[nH]c2c1C(=O)N[C@H]1C[C@@H]21] |
| 171 | 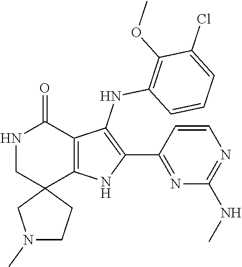 [SMILES: CNc1nccc(-c2[nH]c3c(c2Nc2cccc(Cl)c2OC)C(=O)NCC32CCN(C)C2)n1] |
| 171a | 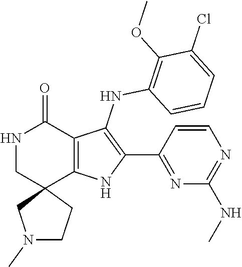 [SMILES: CNc1nccc(-c2[nH]c3c(c2Nc2cccc(Cl)c2OC)C(=O)NC[C@@]32CCN(C)C2)n1] |
| 171b | 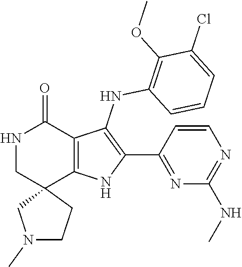 [SMILES: CNc1nccc(-c2[nH]c3c(c2Nc2cccc(Cl)c2OC)C(=O)NC[C@]32CCN(C)C2)n1] |
| 355 | 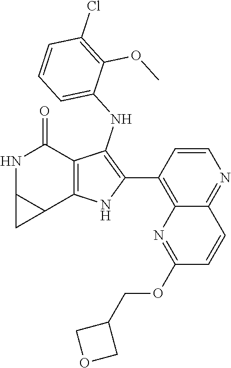 [SMILES: COc1c(Cl)cccc1Nc1c(-c2ccnc3ccc(OCC4COC4)nc23)[nH]c2c1C(=O)NC1CC21] |
| 355a | 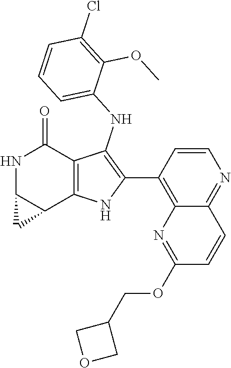 [SMILES: COc1c(Cl)cccc1Nc1c(-c2ccnc3ccc(OCC4COC4)nc23)[nH]c2c1C(=O)N[C@H]1C[C@@H]21] |
| 355b | 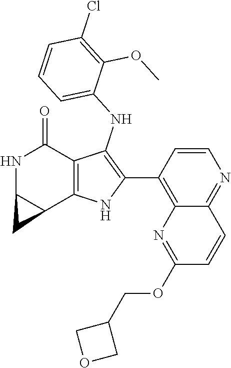 [SMILES: COc1c(Cl)cccc1Nc1c(-c2ccnc3ccc(OCC4COC4)nc23)[nH]c2c1C(=O)N[C@@H]1C[C@H]21] |
| 172 | 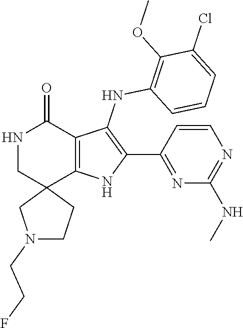 [SMILES: CNc1nccc(-c2[nH]c3c(c2Nc2cccc(Cl)c2OC)C(=O)NCC32CCN(CCF)C2)n1] |
| 172a | 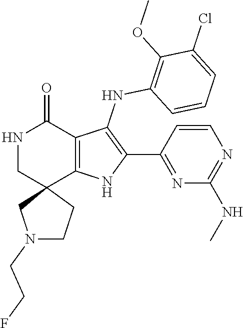 [SMILES: CNc1nccc(-c2[nH]c3c(c2Nc2cccc(Cl)c2OC)C(=O)NC[C@@]32CCN(CCF)C2)n1] |
| 172b | 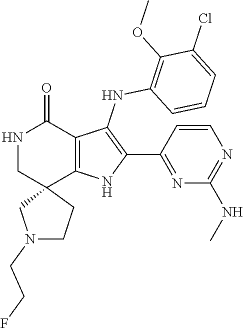 [SMILES: CNc1nccc(-c2[nH]c3c(c2Nc2cccc(Cl)c2OC)C(=O)NC[C@]32CCN(CCF)C2)n1] |
| 356 | 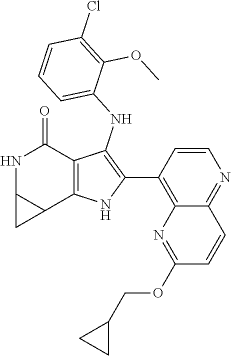 [SMILES: COc1c(Cl)cccc1Nc1c(-c2ccnc3ccc(OCC4CC4)nc23)[nH]c2c1C(=O)NC1CC21] |
| 356a | 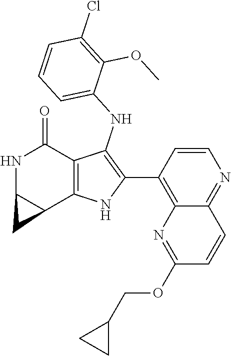 [SMILES: COc1c(Cl)cccc1Nc1c(-c2ccnc3ccc(OCC4CC4)nc23)[nH]c2c1C(=O)N[C@@H]1C[C@H]21] |
| 356b | 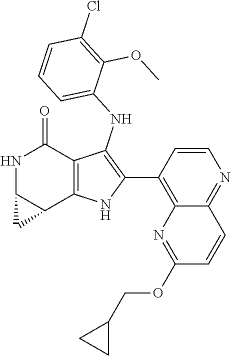 [SMILES: COc1c(Cl)cccc1Nc1c(-c2ccnc3ccc(OCC4CC4)nc23)[nH]c2c1C(=O)N[C@H]1C[C@@H]21] |
| 173 | 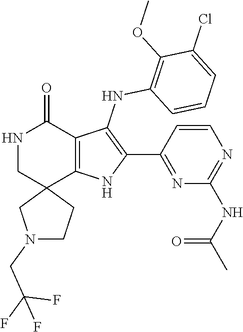 [SMILES: COc1c(Cl)cccc1Nc1c(-c2ccnc(NC(C)=O)n2)[nH]c2c1C(=O)NCC21CCN(CC(F)(F)F)C1] |
| 173a | 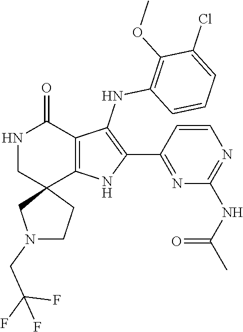 [SMILES: COc1c(Cl)cccc1Nc1c(-c2ccnc(NC(C)=O)n2)[nH]c2c1C(=O)NC[C@@]21CCN(CC(F)(F)F)C1] |
| 173b | 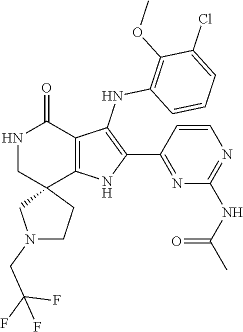 [SMILES: COc1c(Cl)cccc1Nc1c(-c2ccnc(NC(C)=O)n2)[nH]c2c1C(=O)NC[C@]21CCN(CC(F)(F)F)C1] |
| 357 | 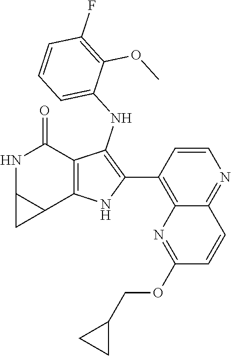 [SMILES: COc1c(F)cccc1Nc1c(-c2ccnc3ccc(OCC4CC4)nc23)[nH]c2c1C(=O)NC1CC21] |
| 357a | 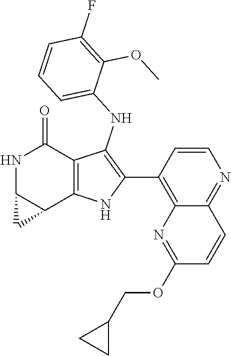 [SMILES: COc1c(F)cccc1Nc1c(-c2ccnc3ccc(OCC4CC4)nc23)[nH]c2c1C(=O)N[C@H]1C[C@@H]21] |
| 357b | 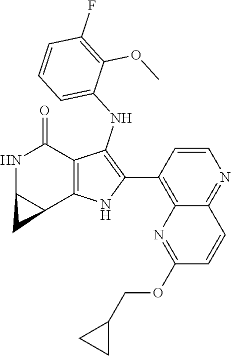 [SMILES: COc1c(F)cccc1Nc1c(-c2ccnc3ccc(OCC4CC4)nc23)[nH]c2c1C(=O)N[C@@H]1C[C@H]21] |
| 174 | 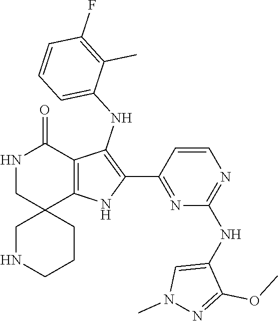 [SMILES: COc1nn(C)cc1Nc1nccc(-c2[nH]c3c(c2Nc2cccc(F)c2C)C(=O)NCC32CCCNC2)n1] |
| 174a | 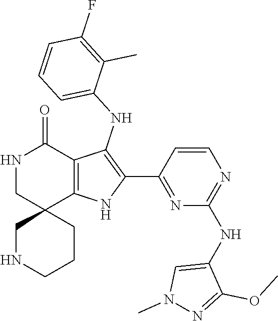 [SMILES: COc1nn(C)cc1Nc1nccc(-c2[nH]c3c(c2Nc2cccc(F)c2C)C(=O)NC[C@@]32CCCNC2)n1] |
| 174b | 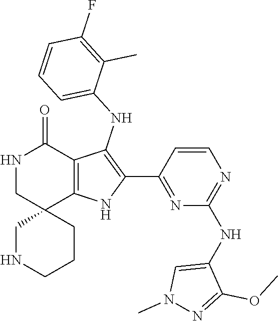 [SMILES: COc1nn(C)cc1Nc1nccc(-c2[nH]c3c(c2Nc2cccc(F)c2C)C(=O)NC[C@]32CCCNC2)n1] |
| 358 | 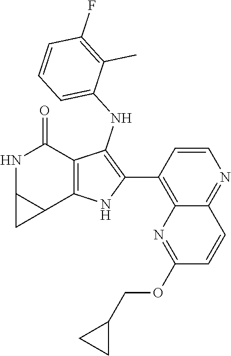 [SMILES: Cc1c(F)cccc1Nc1c(-c2ccnc3ccc(OCC4CC4)nc23)[nH]c2c1C(=O)NC1CC21] |
| 358a | 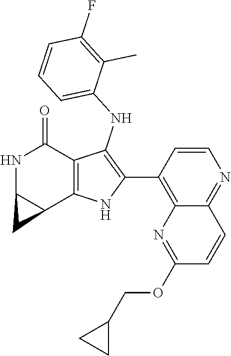 [SMILES: Cc1c(F)cccc1Nc1c(-c2ccnc3ccc(OCC4CC4)nc23)[nH]c2c1C(=O)N[C@@H]1C[C@H]21] |
| 358b | 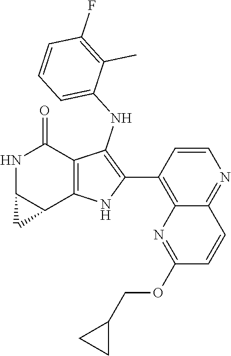 [SMILES: Cc1c(F)cccc1Nc1c(-c2ccnc3ccc(OCC4CC4)nc23)[nH]c2c1C(=O)N[C@H]1C[C@@H]21] |
| 175 | 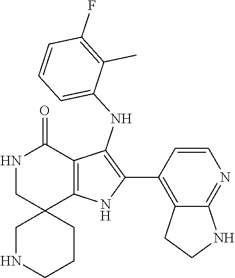 [SMILES: Cc1c(F)cccc1Nc1c(-c2ccnc3c2CCN3)[nH]c2c1C(=O)NCC21CCCNC1] |
| 175a | 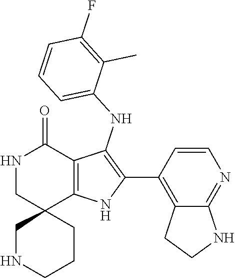 [SMILES: Cc1c(F)cccc1Nc1c(-c2ccnc3c2CCN3)[nH]c2c1C(=O)NC[C@@]21CCCNC1] |
| 175b | 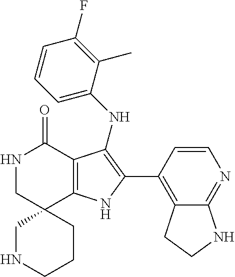 [SMILES: Cc1c(F)cccc1Nc1c(-c2ccnc3c2CCN3)[nH]c2c1C(=O)NC[C@]21CCCNC1] |
| 359 | 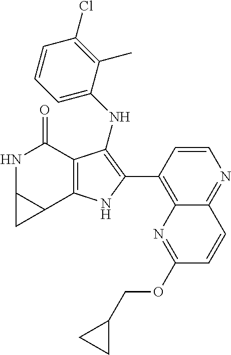 [SMILES: Cc1c(Cl)cccc1Nc1c(-c2ccnc3ccc(OCC4CC4)nc23)[nH]c2c1C(=O)NC1CC21] |
| 359a | 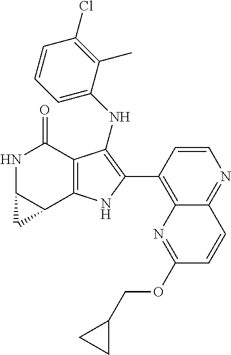 [SMILES: Cc1c(Cl)cccc1Nc1c(-c2ccnc3ccc(OCC4CC4)nc23)[nH]c2c1C(=O)N[C@H]1C[C@@H]21] |
| 359b | 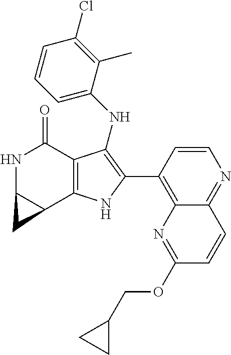 [SMILES: Cc1c(Cl)cccc1Nc1c(-c2ccnc3ccc(OCC4CC4)nc23)[nH]c2c1C(=O)N[C@@H]1C[C@H]21] |
| 176 | 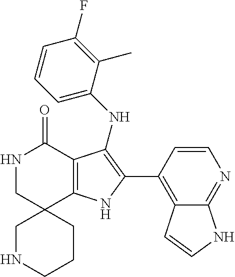 [SMILES: Cc1c(F)cccc1Nc1c(-c2ccnc3[nH]ccc23)[nH]c2c1C(=O)NCC21CCCNC1] |
| 176a | 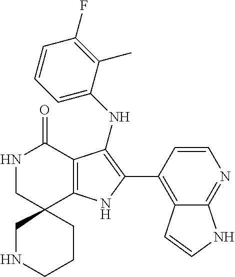 [SMILES: Cc1c(F)cccc1Nc1c(-c2ccnc3[nH]ccc23)[nH]c2c1C(=O)NC[C@@]21CCCNC1] |
| 176b | 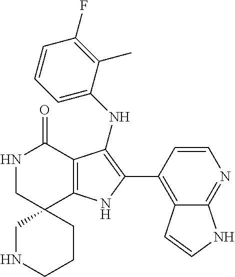 [SMILES: Cc1c(F)cccc1Nc1c(-c2ccnc3[nH]ccc23)[nH]c2c1C(=O)NC[C@]21CCCNC1] |
| 360 | 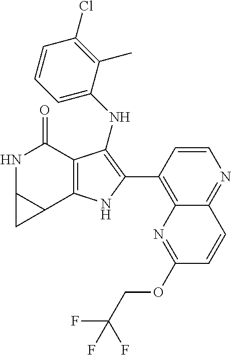 [SMILES: Cc1c(Cl)cccc1Nc1c(-c2ccnc3ccc(OCC(F)(F)F)nc23)[nH]c2c1C(=O)NC1CC21] |
| 360a | 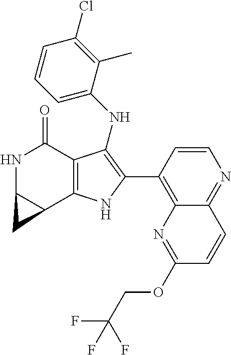 [SMILES: Cc1c(Cl)cccc1Nc1c(-c2ccnc3ccc(OCC(F)(F)F)nc23)[nH]c2c1C(=O)N[C@@H]1C[C@H]21] |
| 360b | 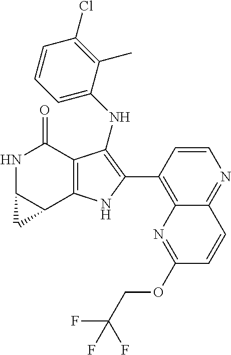 [SMILES: Cc1c(Cl)cccc1Nc1c(-c2ccnc3ccc(OCC(F)(F)F)nc23)[nH]c2c1C(=O)N[C@H]1C[C@@H]21] |
| 177 | 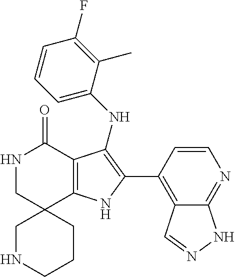 [SMILES: Cc1c(F)cccc1Nc1c(-c2ccnc3[nH]ncc23)[nH]c2c1C(=O)NCC21CCCNC1] |
| 177a | 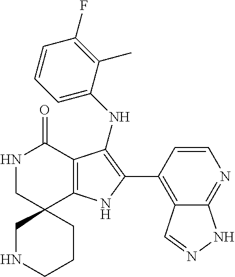 [SMILES: Cc1c(F)cccc1Nc1c(-c2ccnc3[nH]ncc23)[nH]c2c1C(=O)NC[C@@]21CCCNC1] |
| 177b | 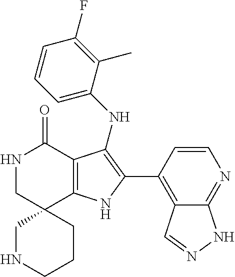 [SMILES: Cc1c(F)cccc1Nc1c(-c2ccnc3[nH]ncc23)[nH]c2c1C(=O)NC[C@]21CCCNC1] |
| 361 | 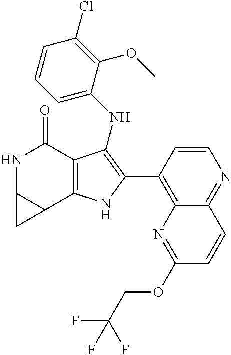 [SMILES: COc1c(Cl)cccc1Nc1c(-c2ccnc3ccc(OCC(F)(F)F)nc23)[nH]c2c1C(=O)NC1CC21] |
| 361a | 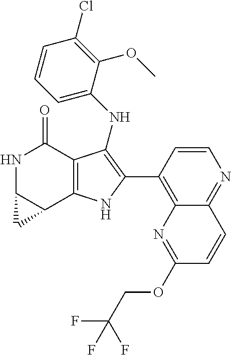 [SMILES: COc1c(Cl)cccc1Nc1c(-c2ccnc3ccc(OCC(F)(F)F)nc23)[nH]c2c1C(=O)N[C@H]1C[C@@H]21] |
| 361b | 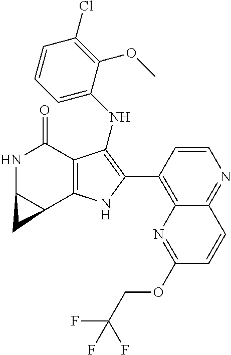 [SMILES: COc1c(Cl)cccc1Nc1c(-c2ccnc3ccc(OCC(F)(F)F)nc23)[nH]c2c1C(=O)N[C@@H]1C[C@H]21] |
| 178 | 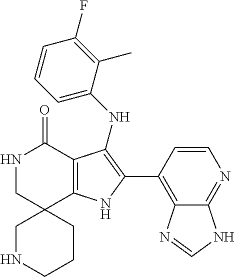 [SMILES: Cc1c(F)cccc1Nc1c(-c2ccnc3[nH]cnc23)[nH]c2c1C(=O)NCC21CCCNC1] |
| 178a | 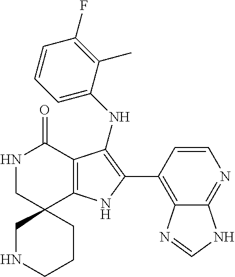 [SMILES: Cc1c(F)cccc1Nc1c(-c2ccnc3[nH]cnc23)[nH]c2c1C(=O)NC[C@@]21CCCNC1] |
| 178b | 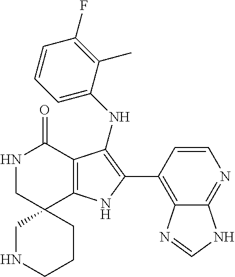 [SMILES: Cc1c(F)cccc1Nc1c(-c2ccnc3[nH]cnc23)[nH]c2c1C(=O)NC[C@]21CCCNC1] |
| 362 | 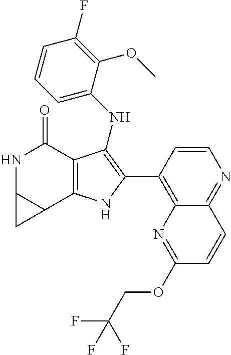 [SMILES: COc1c(F)cccc1Nc1c(-c2ccnc3ccc(OCC(F)(F)F)nc23)[nH]c2c1C(=O)NC1CC21] |
| 362a | 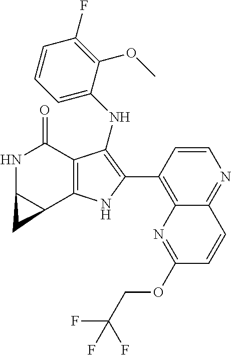 [SMILES: COc1c(F)cccc1Nc1c(-c2ccnc3ccc(OCC(F)(F)F)nc23)[nH]c2c1C(=O)N[C@@H]1C[C@H]21] |
| 362b | 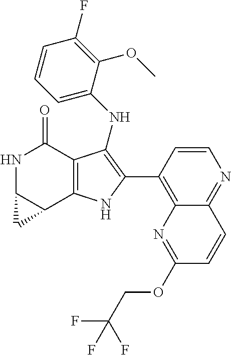 [SMILES: COc1c(F)cccc1Nc1c(-c2ccnc3ccc(OCC(F)(F)F)nc23)[nH]c2c1C(=O)N[C@H]1C[C@@H]21] |
| 179 | 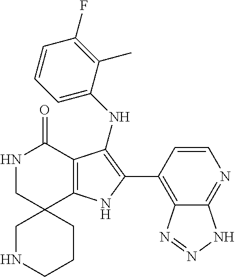 [SMILES: Cc1c(F)cccc1Nc1c(-c2ccnc3[nH]nnc23)[nH]c2c1C(=O)NCC21CCCNC1] |
| 179a | 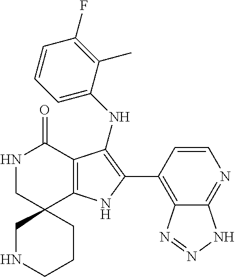 [SMILES: Cc1c(F)cccc1Nc1c(-c2ccnc3[nH]nnc23)[nH]c2c1C(=O)NC[C@@]21CCCNC1] |
| 179b | 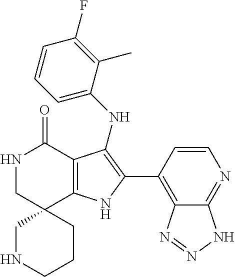 [SMILES: Cc1c(F)cccc1Nc1c(-c2ccnc3[nH]nnc23)[nH]c2c1C(=O)NC[C@]21CCCNC1] |
| 180 | 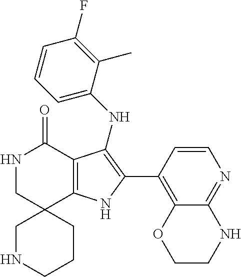 [SMILES: Cc1c(F)cccc1Nc1c(-c2ccnc3c2OCCN3)[nH]c2c1C(=O)NCC21CCCNC1] |
| 363 | 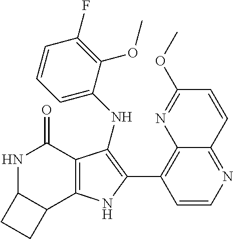 [SMILES: COc1ccc2nccc(-c3[nH]c4c(c3Nc3cccc(F)c3OC)C(=O)NC3CCC43)c2n1] |
| 363a | 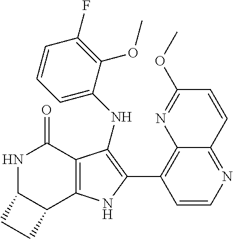 [SMILES: COc1ccc2nccc(-c3[nH]c4c(c3Nc3cccc(F)c3OC)C(=O)N[C@H]3CC[C@@H]43)c2n1] |
| 363b | 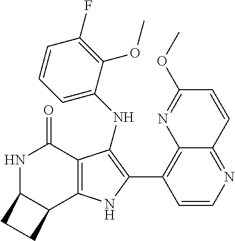 [SMILES: COc1ccc2nccc(-c3[nH]c4c(c3Nc3cccc(F)c3OC)C(=O)N[C@@H]3CC[C@H]43)c2n1] |
| 364 | 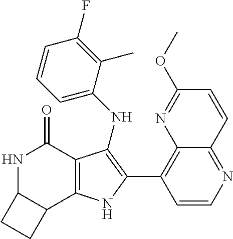 [SMILES: COc1ccc2nccc(-c3[nH]c4c(c3Nc3cccc(F)c3C)C(=O)NC3CCC43)c2n1] |
| 180a | 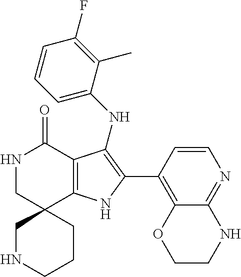 [SMILES: Cc1c(F)cccc1Nc1c(-c2ccnc3c2OCCN3)[nH]c2c1C(=O)NC[C@@]21CCCNC1] |
| 180b | 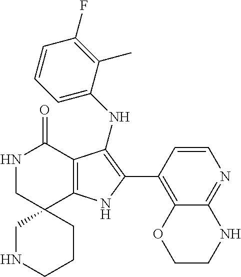 [SMILES: Cc1c(F)cccc1Nc1c(-c2ccnc3c2OCCN3)[nH]c2c1C(=O)NC[C@]21CCCNC1] |
| 181 | 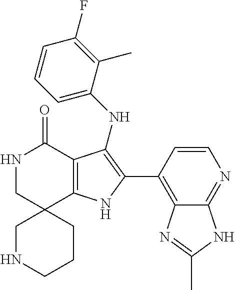 [SMILES: Cc1nc2c(-c3[nH]c4c(c3Nc3cccc(F)c3C)C(=O)NCC43CCCNC3)ccnc2[nH]1] |
| 181a | 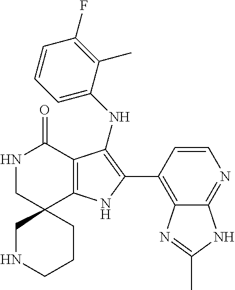 [SMILES: Cc1nc2c(-c3[nH]c4c(c3Nc3cccc(F)c3C)C(=O)NC[C@@]43CCCNC3)ccnc2[nH]1] |
| 364a | 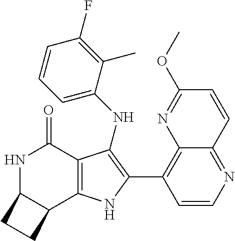 [SMILES: COc1ccc2nccc(-c3[nH]c4c(c3Nc3cccc(F)c3C)C(=O)N[C@@H]3CC[C@H]43)c2n1] |
| 364b | 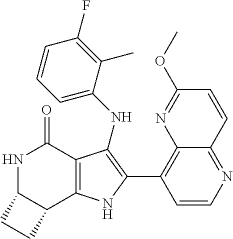 [SMILES: COc1ccc2nccc(-c3[nH]c4c(c3Nc3cccc(F)c3C)C(=O)N[C@H]3CC[C@@H]43)c2n1] |
| 365 | 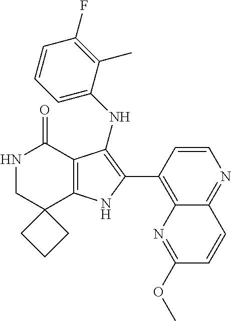 [SMILES: COc1ccc2nccc(-c3[nH]c4c(c3Nc3cccc(F)c3C)C(=O)NCC43CCC3)c2n1] |
| 366 | 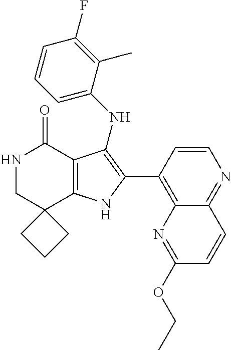 [SMILES: CCOc1ccc2nccc(-c3[nH]c4c(c3Nc3cccc(F)c3C)C(=O)NCC43CCC3)c2n1] |
| 181b | 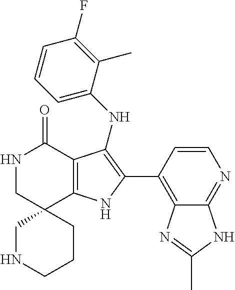 [SMILES: Cc1nc2c(-c3[nH]c4c(c3Nc3cccc(F)c3C)C(=O)NC[C@]43CCCNC3)ccnc2[nH]1] |
| 182 | 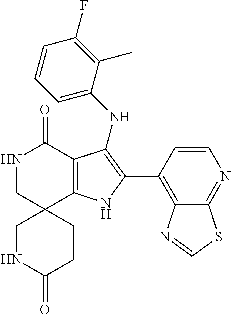 [SMILES: Cc1c(F)cccc1Nc1c(-c2ccnc3scnc23)[nH]c2c1C(=O)NCC21CCC(=O)NC1] |
| 182a | 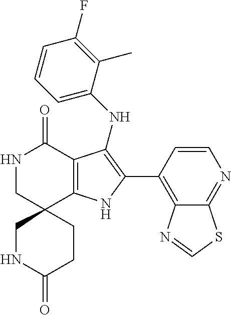 [SMILES: Cc1c(F)cccc1Nc1c(-c2ccnc3scnc23)[nH]c2c1C(=O)NC[C@@]21CCC(=O)NC1] |
| 182b | 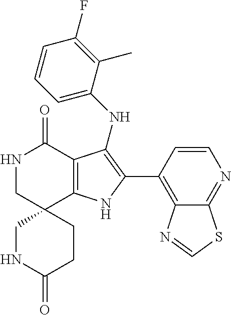 [SMILES: Cc1c(F)cccc1Nc1c(-c2ccnc3scnc23)[nH]c2c1C(=O)NC[C@]21CCC(=O)NC1] |
| 367 | 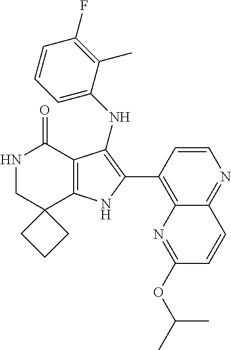 [SMILES: Cc1c(F)cccc1Nc1c(-c2ccnc3ccc(OC(C)C)nc23)[nH]c2c1C(=O)NCC21CCC1] |
| 368 | 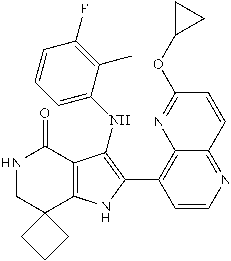 [SMILES: Cc1c(F)cccc1Nc1c(-c2ccnc3ccc(OC4CC4)nc23)[nH]c2c1C(=O)NCC21CCC1] |
| 369 | 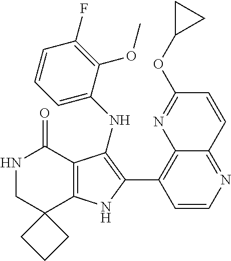 [SMILES: COc1c(F)cccc1Nc1c(-c2ccnc3ccc(OC4CC4)nc23)[nH]c2c1C(=O)NCC21CCC1] |
| 370 | 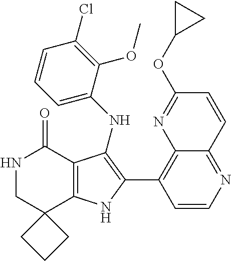 [SMILES: COc1c(Cl)cccc1Nc1c(-c2ccnc3ccc(OC4CC4)nc23)[nH]c2c1C(=O)NCC21CCC1] |
| 183 | 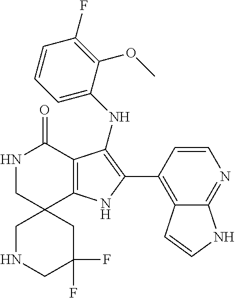 [SMILES: COc1c(F)cccc1Nc1c(-c2ccnc3[nH]ccc23)[nH]c2c1C(=O)NCC21CNCC(F)(F)C1] |
| 183a | 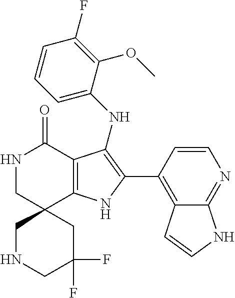 [SMILES: COc1c(F)cccc1Nc1c(-c2ccnc3[nH]ccc23)[nH]c2c1C(=O)NC[C@]21CNCC(F)(F)C1] |
| 183b | 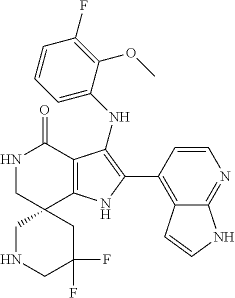 [SMILES: COc1c(F)cccc1Nc1c(-c2ccnc3[nH]ccc23)[nH]c2c1C(=O)NC[C@@]21CNCC(F)(F)C1] |
| 184 | 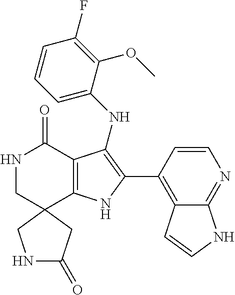 [SMILES: COc1c(F)cccc1Nc1c(-c2ccnc3[nH]ccc23)[nH]c2c1C(=O)NCC21CNC(=O)C1] |
| 371 | 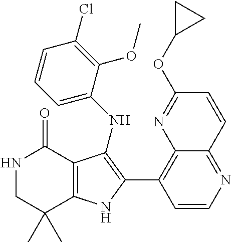 [SMILES: COc1c(Cl)cccc1Nc1c(-c2ccnc3ccc(OC4CC4)nc23)[nH]c2c1C(=O)NCC2(C)C] |
| 372 | 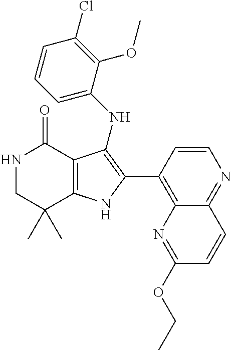 [SMILES: CCOc1ccc2nccc(-c3[nH]c4c(c3Nc3cccc(Cl)c3OC)C(=O)NCC4(C)C)c2n1] |
| 373 | 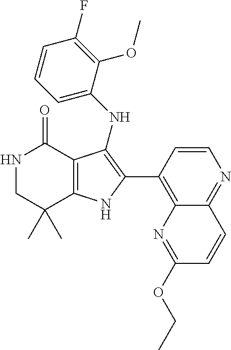 [SMILES: CCOc1ccc2nccc(-c3[nH]c4c(c3Nc3cccc(F)c3OC)C(=O)NCC4(C)C)c2n1] |
| 374 | 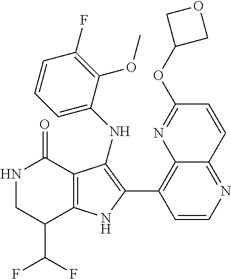 [SMILES: COc1c(F)cccc1Nc1c(-c2ccnc3ccc(OC4COC4)nc23)[nH]c2c1C(=O)NCC2C(F)F] |
| 184a | 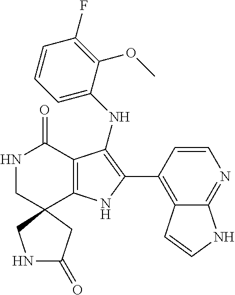 [SMILES: COc1c(F)cccc1Nc1c(-c2ccnc3[nH]ccc23)[nH]c2c1C(=O)NC[C@]21CNC(=O)C1] |
| 184b | 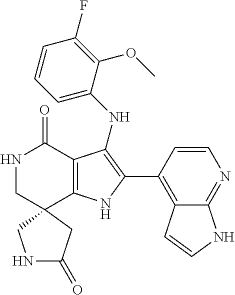 [SMILES: COc1c(F)cccc1Nc1c(-c2ccnc3[nH]ccc23)[nH]c2c1C(=O)NC[C@@]21CNC(=O)C1] |
| 185 | 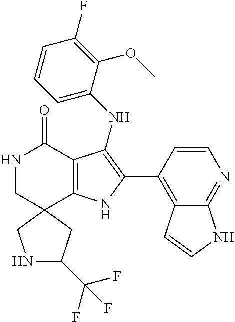 [SMILES: COc1c(F)cccc1Nc1c(-c2ccnc3[nH]ccc23)[nH]c2c1C(=O)NCC21CNC(C(F)(F)F)C1] |
| 185a | 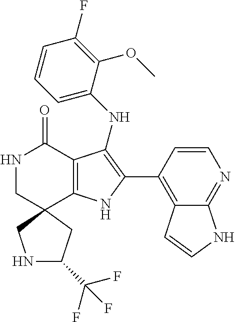 [SMILES: COc1c(F)cccc1Nc1c(-c2ccnc3[nH]ccc23)[nH]c2c1C(=O)NC[C@]21CN[C@@H](C(F)(F)F)C1] |
| 374a | 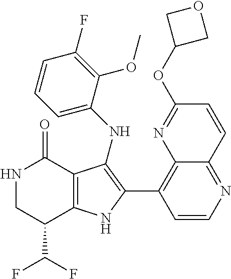 [SMILES: COc1c(F)cccc1Nc1c(-c2ccnc3ccc(OC4COC4)nc23)[nH]c2c1C(=O)NC[C@H]2C(F)F] |
| 374b | 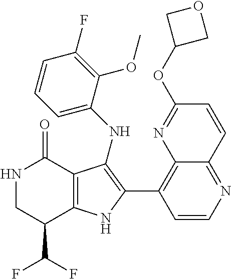 [SMILES: COc1c(F)cccc1Nc1c(-c2ccnc3ccc(OC4COC4)nc23)[nH]c2c1C(=O)NC[C@@H]2C(F)F] |
| 375 | 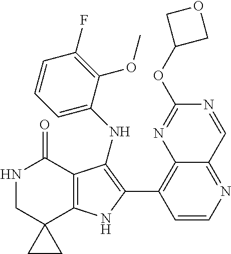 [SMILES: COc1c(F)cccc1Nc1c(-c2ccnc3cnc(OC4COC4)nc23)[nH]c2c1C(=O)NCC21CC1] |
| 376 | 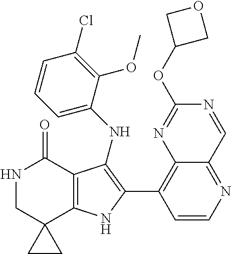 [SMILES: COc1c(Cl)cccc1Nc1c(-c2ccnc3cnc(OC4COC4)nc23)[nH]c2c1C(=O)NCC21CC1] |
| 185b | 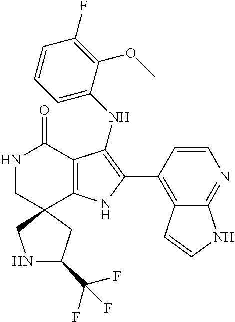 [SMILES: COc1c(F)cccc1Nc1c(-c2ccnc3[nH]ccc23)[nH]c2c1C(=O)NC[C@]21CN[C@H](C(F)(F)F)C1] |
| 185c | 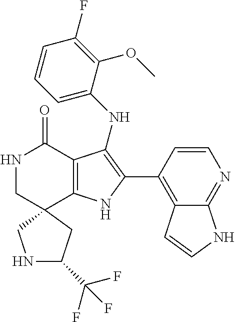 [SMILES: COc1c(F)cccc1Nc1c(-c2ccnc3[nH]ccc23)[nH]c2c1C(=O)NC[C@@]21CN[C@@H](C(F)(F)F)C1] |
| 185d | 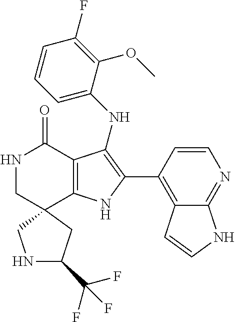 [SMILES: COc1c(F)cccc1Nc1c(-c2ccnc3[nH]ccc23)[nH]c2c1C(=O)NC[C@@]21CN[C@H](C(F)(F)F)C1] |
| 377 | 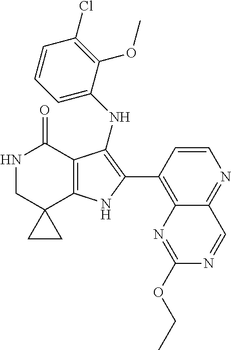 [SMILES: CCOc1ncc2nccc(-c3[nH]c4c(c3Nc3cccc(Cl)c3OC)C(=O)NCC43CC3)c2n1] |
| 378 | 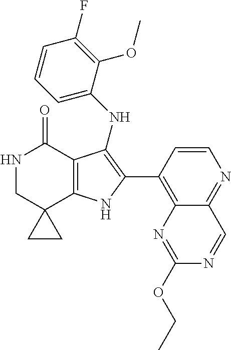 [SMILES: CCOc1ncc2nccc(-c3[nH]c4c(c3Nc3cccc(F)c3OC)C(=O)NCC43CC3)c2n1] |
| 379 | 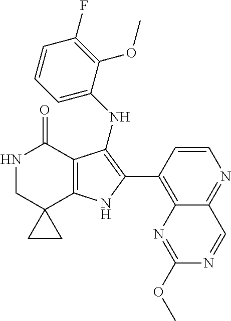 [SMILES: COc1ncc2nccc(-c3[nH]c4c(c3Nc3cccc(F)c3OC)C(=O)NCC43CC3)c2n1] |
| 186 | 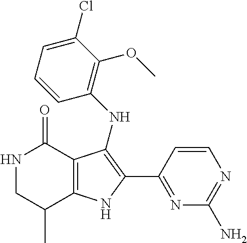 [SMILES: COc1c(Cl)cccc1Nc1c(-c2ccnc(N)n2)[nH]c2c1C(=O)NCC2C] |
| 186a | 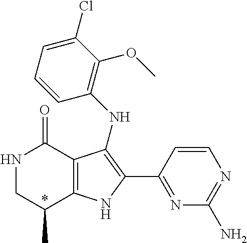 [SMILES: COc1c(Cl)cccc1Nc1c(-c2ccnc(N)n2)[nH]c2c1C(=O)NC[C@@H]2C] |
| 186b | 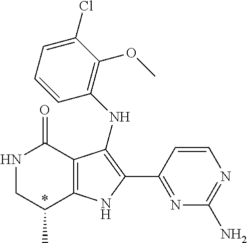 [SMILES: COc1c(Cl)cccc1Nc1c(-c2ccnc(N)n2)[nH]c2c1C(=O)NC[C@H]2C] |
| 187 | 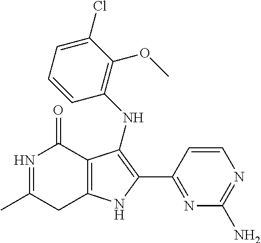 [SMILES: COc1c(Cl)cccc1Nc1c(-c2ccnc(N)n2)[nH]c2c1C(=O)N=C(C)C2] |
| 380 | 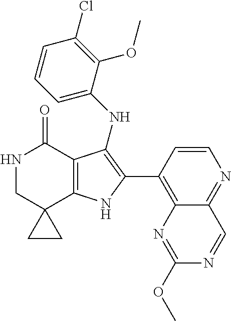 [SMILES: COc1ncc2nccc(-c3[nH]c4c(c3Nc3cccc(Cl)c3OC)C(=O)NCC43CC3)c2n1] |
| 381 | 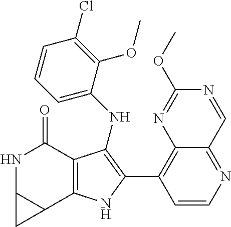 [SMILES: COc1ncc2nccc(-c3[nH]c4c(c3Nc3cccc(Cl)c3OC)C(=O)NC3CC43)c2n1] |
| 381a | 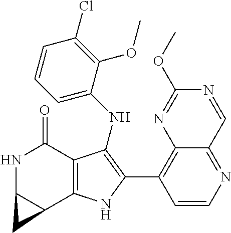 [SMILES: COc1ncc2nccc(-c3[nH]c4c(c3Nc3cccc(Cl)c3OC)C(=O)N[C@@H]3C[C@H]43)c2n1] |
| 381b | 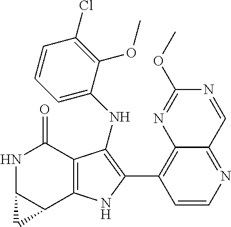 [SMILES: COc1ncc2nccc(-c3[nH]c4c(c3Nc3cccc(Cl)c3OC)C(=O)N[C@H]3C[C@@H]43)c2n1] |
| 187a | 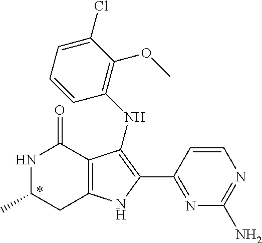 [SMILES: COc1c(Cl)cccc1Nc1c(-c2ccnc(N)n2)[nH]c2c1C(=O)N[C@@H](C)C2] |
| 187b | 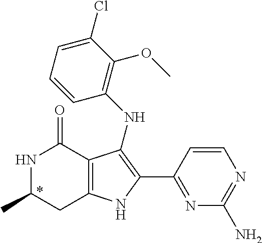 [SMILES: COc1c(Cl)cccc1Nc1c(-c2ccnc(N)n2)[nH]c2c1C(=O)N[C@H](C)C2] |
| 188 | 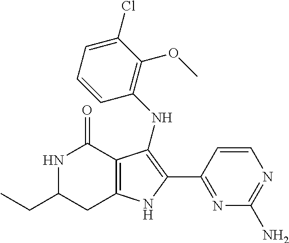 [SMILES: CCC1Cc2[nH]c(-c3ccnc(N)n3)c(Nc3cccc(Cl)c3OC)c2C(=O)N1] |
| 188a | 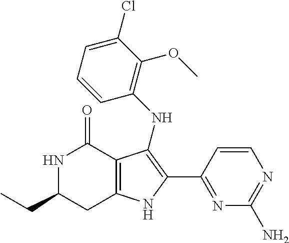 [SMILES: CC[C@@H]1Cc2[nH]c(-c3ccnc(N)n3)c(Nc3cccc(Cl)c3OC)c2C(=O)N1] |
| 188b | 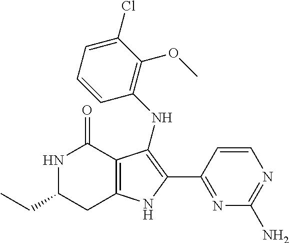 [SMILES: CC[C@H]1Cc2[nH]c(-c3ccnc(N)n3)c(Nc3cccc(Cl)c3OC)c2C(=O)N1] |
| 382 | 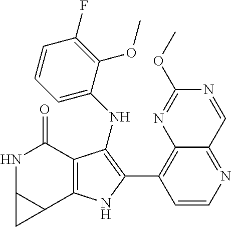 [SMILES: COc1ncc2nccc(-c3[nH]c4c(c3Nc3cccc(F)c3OC)C(=O)NC3CC43)c2n1] |
| 382a | 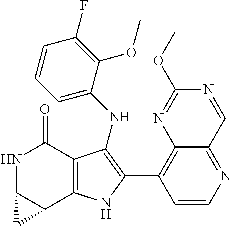 [SMILES: COc1ncc2nccc(-c3[nH]c4c(c3Nc3cccc(F)c3OC)C(=O)N[C@H]3C[C@@H]43)c2n1] |
| 382b | 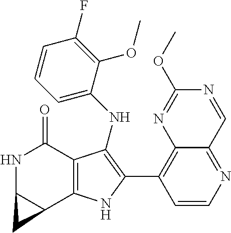 [SMILES: COc1ncc2nccc(-c3[nH]c4c(c3Nc3cccc(F)c3OC)C(=O)N[C@@H]3C[C@H]43)c2n1] |
| 383 | 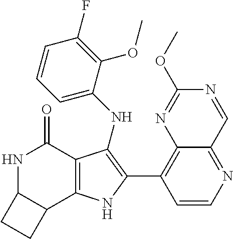 [SMILES: COc1ncc2nccc(-c3[nH]c4c(c3Nc3cccc(F)c3OC)C(=O)NC3CCC43)c2n1] |
| 383a | 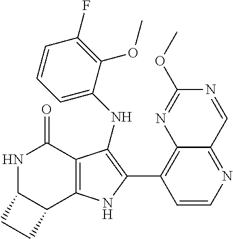 [SMILES: COc1ncc2nccc(-c3[nH]c4c(c3Nc3cccc(F)c3OC)C(=O)N[C@H]3CC[C@@H]43)c2n1] |
| 189 | 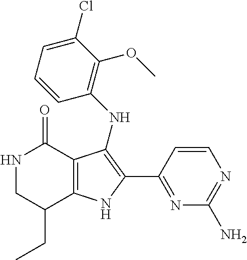 [SMILES: CCC1CNC(=O)c2c1[nH]c(-c1ccnc(N)n1)c2Nc1cccc(Cl)c1OC] |
| 189a | 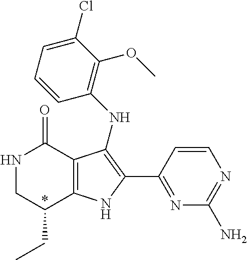 [SMILES: CC[C@@H]1CNC(=O)c2c1[nH]c(-c1ccnc(N)n1)c2Nc1cccc(Cl)c1OC] |
| 189b | 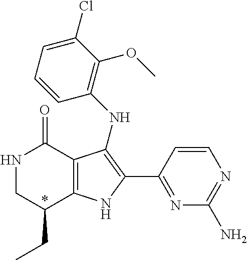 [SMILES: CC[C@H]1CNC(=O)c2c1[nH]c(-c1ccnc(N)n1)c2Nc1cccc(Cl)c1OC] |
| 190 | 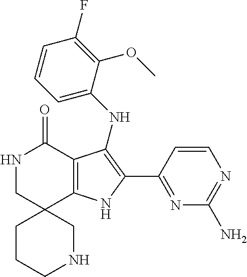 [SMILES: COc1c(F)cccc1Nc1c(-c2ccnc(N)n2)[nH]c2c1C(=O)NCC21CCCNC1] |
| 383b | 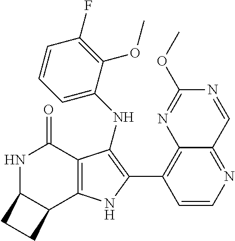 [SMILES: COc1ncc2nccc(-c3[nH]c4c(c3Nc3cccc(F)c3OC)C(=O)N[C@@H]3CC[C@H]43)c2n1] |
| 384 | 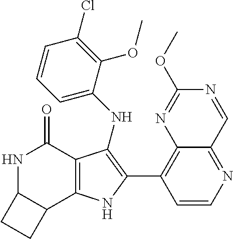 [SMILES: COc1ncc2nccc(-c3[nH]c4c(c3Nc3cccc(Cl)c3OC)C(=O)NC3CCC43)c2n1] |
| 384a | 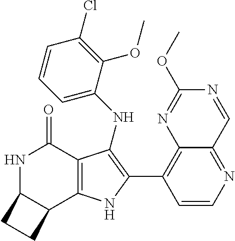 [SMILES: COc1ncc2nccc(-c3[nH]c4c(c3Nc3cccc(Cl)c3OC)C(=O)N[C@@H]3CC[C@H]43)c2n1] |
| 384b | 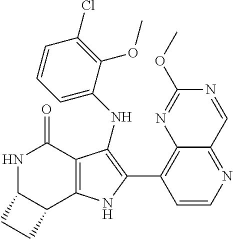 [SMILES: COc1ncc2nccc(-c3[nH]c4c(c3Nc3cccc(Cl)c3OC)C(=O)N[C@H]3CC[C@@H]43)c2n1] |
| 190a | 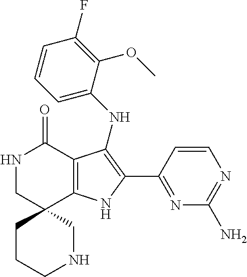 [SMILES: COc1c(F)cccc1Nc1c(-c2ccnc(N)n2)[nH]c2c1C(=O)NC[C@]21CCCNC1] |
| 190b | 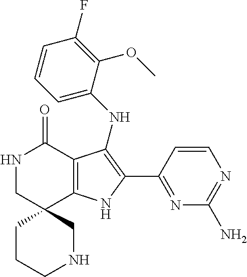 [SMILES: COc1c(F)cccc1Nc1c(-c2ccnc(N)n2)[nH]c2c1C(=O)NC[C@@]21CCCNC1] |
| 191 | 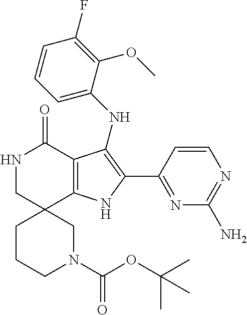 [SMILES: COc1c(F)cccc1Nc1c(-c2ccnc(N)n2)[nH]c2c1C(=O)NCC21CCCN(C(=O)OC(C)(C)C)C1] |
| 191a | 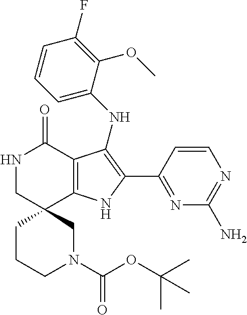 [SMILES: COc1c(F)cccc1Nc1c(-c2ccnc(N)n2)[nH]c2c1C(=O)NC[C@@]21CCCN(C(=O)OC(C)(C)C)C1] |
| 385 | 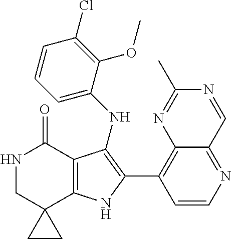 [SMILES: COc1c(Cl)cccc1Nc1c(-c2ccnc3cnc(C)nc23)[nH]c2c1C(=O)NCC21CC1] |
| 386 | 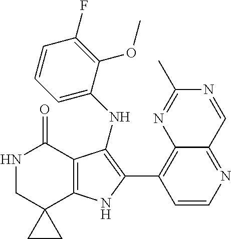 [SMILES: COc1c(F)cccc1Nc1c(-c2ccnc3cnc(C)nc23)[nH]c2c1C(=O)NCC21CC1] |
| 387 | 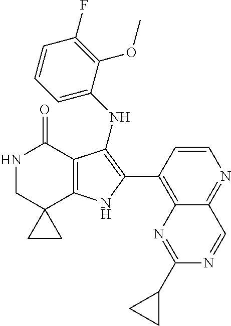 [SMILES: COc1c(F)cccc1Nc1c(-c2ccnc3cnc(C4CC4)nc23)[nH]c2c1C(=O)NCC21CC1] |
| 388 | 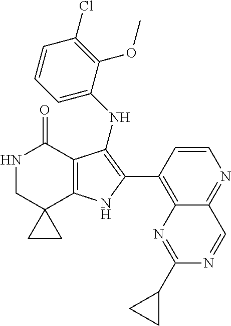 [SMILES: COc1c(Cl)cccc1Nc1c(-c2ccnc3cnc(C4CC4)nc23)[nH]c2c1C(=O)NCC21CC1] |
| 191b | 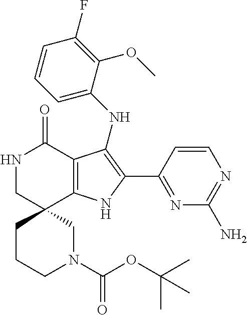 [SMILES: COc1c(F)cccc1Nc1c(-c2ccnc(N)n2)[nH]c2c1C(=O)NC[C@]21CCCN(C(=O)OC(C)(C)C)C1] |
| 192 | 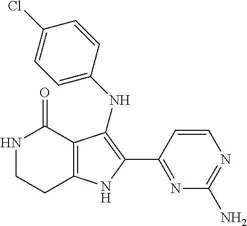 [SMILES: Nc1nccc(-c2[nH]c3c(c2Nc2ccc(Cl)cc2)C(=O)NCC3)n1] |
| 193 | 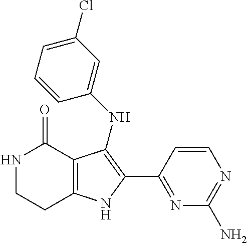 [SMILES: Nc1nccc(-c2[nH]c3c(c2Nc2cccc(Cl)c2)C(=O)NCC3)n1] |
| 389 | 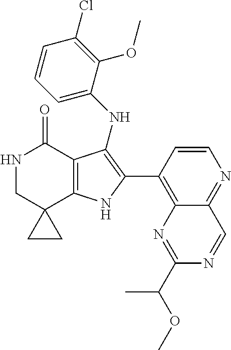 [SMILES: COc1c(Cl)cccc1Nc1c(-c2ccnc3cnc(C(C)OC)nc23)[nH]c2c1C(=O)NCC21CC1] |
| 389a | 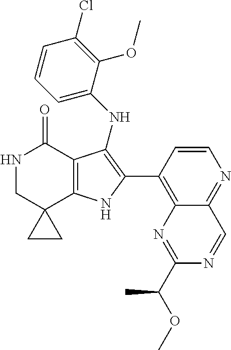 [SMILES: COc1c(Cl)cccc1Nc1c(-c2ccnc3cnc([C@H](C)OC)nc23)[nH]c2c1C(=O)NCC21CC1] |
| 389b | 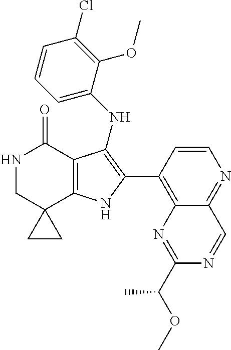 [SMILES: COc1c(Cl)cccc1Nc1c(-c2ccnc3cnc([C@@H](C)OC)nc23)[nH]c2c1C(=O)NCC21CC1] |
| 194 | 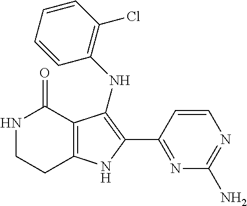 [SMILES: Nc1nccc(-c2[nH]c3c(c2Nc2ccccc2Cl)C(=O)NCC3)n1] |
| 195 | 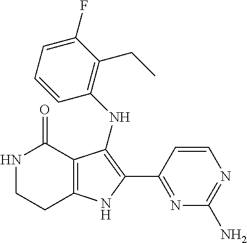 [SMILES: CCc1c(F)cccc1Nc1c(-c2ccnc(N)n2)[nH]c2c1C(=O)NCC2] |
| 196 | 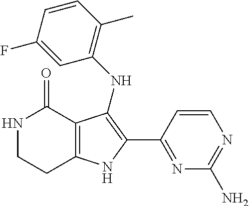 [SMILES: Cc1ccc(F)cc1Nc1c(-c2ccnc(N)n2)[nH]c2c1C(=O)NCC2] |
| 390 | 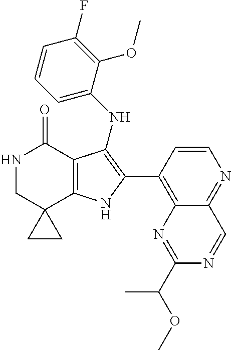 [SMILES: COc1c(F)cccc1Nc1c(-c2ccnc3cnc(C(C)OC)nc23)[nH]c2c1C(=O)NCC21CC1] |
| 390a | 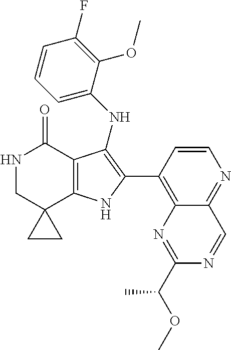 [SMILES: COc1c(F)cccc1Nc1c(-c2ccnc3cnc([C@@H](C)OC)nc23)[nH]c2c1C(=O)NCC21CC1] |
| 390b | 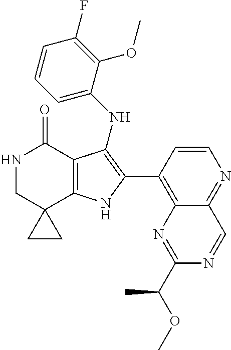 [SMILES: COc1c(F)cccc1Nc1c(-c2ccnc3cnc([C@H](C)OC)nc23)[nH]c2c1C(=O)NCC21CC1] |
| 197 | 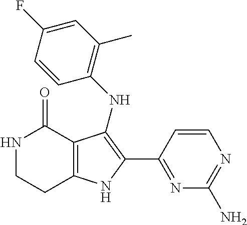 [SMILES: Cc1cc(F)ccc1Nc1c(-c2ccnc(N)n2)[nH]c2c1C(=O)NCC2] |
| 198 | 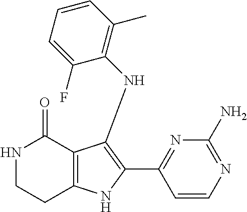 [SMILES: Cc1cccc(F)c1Nc1c(-c2ccnc(N)n2)[nH]c2c1C(=O)NCC2] |
| 199 | 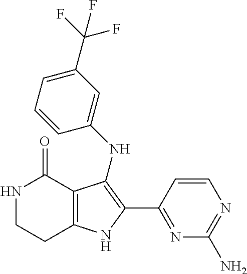 [SMILES: Nc1nccc(-c2[nH]c3c(c2Nc2cccc(C(F)(F)F)c2)C(=O)NCC3)n1] |
| 200 | 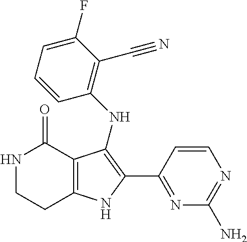 [SMILES: N#Cc1c(F)cccc1Nc1c(-c2ccnc(N)n2)[nH]c2c1C(=O)NCC2] |
| 391 | 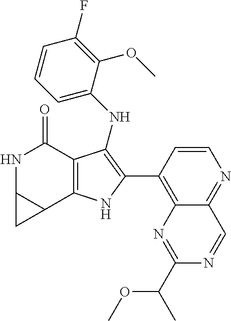 [SMILES: COc1c(F)cccc1Nc1c(-c2ccnc3cnc(C(C)OC)nc23)[nH]c2c1C(=O)NC1CC21] |
| 391a | 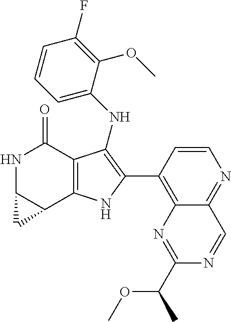 [SMILES: COc1c(F)cccc1Nc1c(-c2ccnc3cnc([C@@H](C)OC)nc23)[nH]c2c1C(=O)N[C@H]1C[C@@H]21] |
| 391b | 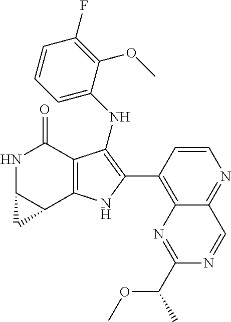 [SMILES: COc1c(F)cccc1Nc1c(-c2ccnc3cnc([C@H](C)OC)nc23)[nH]c2c1C(=O)N[C@H]1C[C@@H]21] |
| 391c | 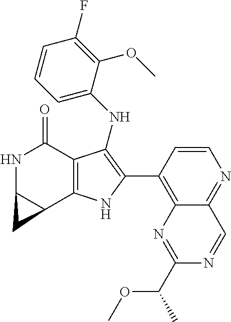 [SMILES: COc1c(F)cccc1Nc1c(-c2ccnc3cnc([C@H](C)OC)nc23)[nH]c2c1C(=O)N[C@@H]1C[C@H]21] |
| 201 | 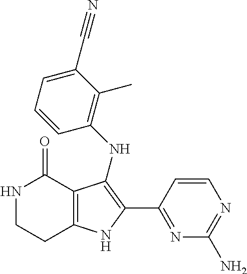 [SMILES: Cc1c(C#N)cccc1Nc1c(-c2ccnc(N)n2)[nH]c2c1C(=O)NCC2] |
| 202 | 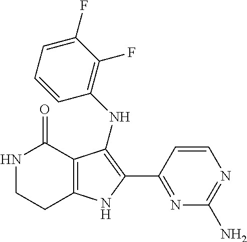 [SMILES: Nc1nccc(-c2[nH]c3c(c2Nc2cccc(F)c2F)C(=O)NCC3)n1] |
| 203 | 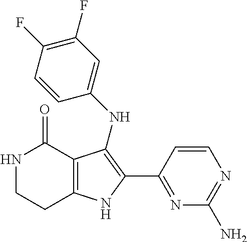 [SMILES: Nc1nccc(-c2[nH]c3c(c2Nc2ccc(F)c(F)c2)C(=O)NCC3)n1] |
| 204 | 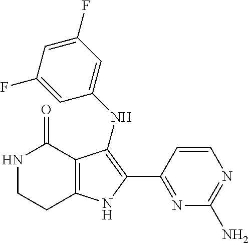 [SMILES: Nc1nccc(-c2[nH]c3c(c2Nc2cc(F)cc(F)c2)C(=O)NCC3)n1] |
| 391d | 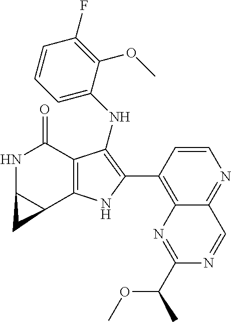 [SMILES: COc1c(F)cccc1Nc1c(-c2ccnc3cnc([C@@H](C)OC)nc23)[nH]c2c1C(=O)N[C@@H]1C[C@H]21] |
| 392 | 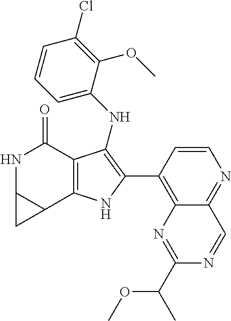 [SMILES: COc1c(Cl)cccc1Nc1c(-c2ccnc3cnc(C(C)OC)nc23)[nH]c2c1C(=O)NC1CC21] |
| 392a | 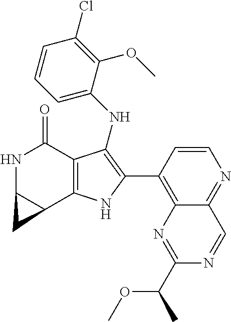 [SMILES: COc1c(Cl)cccc1Nc1c(-c2ccnc3cnc([C@@H](C)OC)nc23)[nH]c2c1C(=O)N[C@@H]1C[C@H]21] |
| 392b | 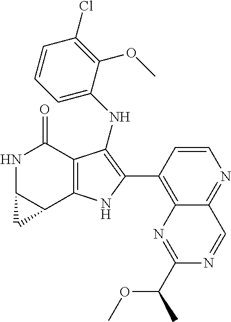 [SMILES: COc1c(Cl)cccc1Nc1c(-c2ccnc3cnc([C@@H](C)OC)nc23)[nH]c2c1C(=O)N[C@H]1C[C@@H]21] |
| 205 | 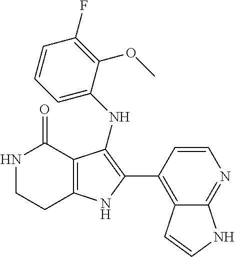 [SMILES: COc1c(F)cccc1Nc1c(-c2ccnc3[nH]ccc23)[nH]c2c1C(=O)NCC2] |
| 206 | 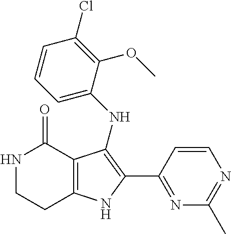 [SMILES: COc1c(Cl)cccc1Nc1c(-c2ccnc(C)n2)[nH]c2c1C(=O)NCC2] |
| 207 | 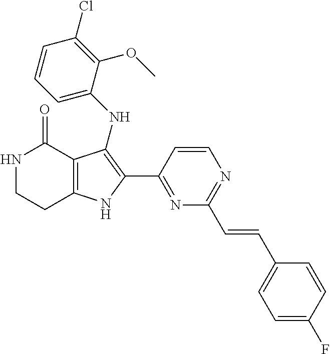 [SMILES: COc1c(Cl)cccc1Nc1c(-c2ccnc(/C=C/c3ccc(F)cc3)n2)[nH]c2c1C(=O)NCC2] |
| 392c | 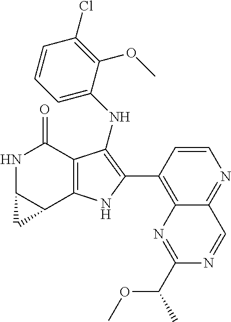 [SMILES: COc1c(Cl)cccc1Nc1c(-c2ccnc3cnc([C@H](C)OC)nc23)[nH]c2c1C(=O)N[C@H]1C[C@@H]21] |
| 392d | 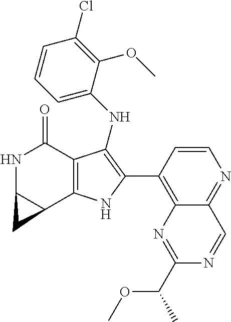 [SMILES: COc1c(Cl)cccc1Nc1c(-c2ccnc3cnc([C@H](C)OC)nc23)[nH]c2c1C(=O)N[C@@H]1C[C@H]21] |
| 393 | 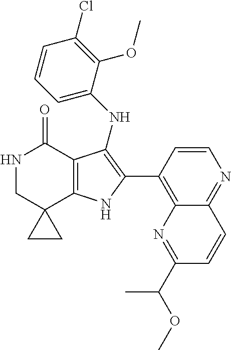 [SMILES: COc1c(Cl)cccc1Nc1c(-c2ccnc3ccc(C(C)OC)nc23)[nH]c2c1C(=O)NCC21CC1] |
| 208 | 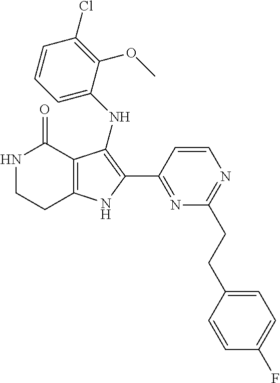 [SMILES: COc1c(Cl)cccc1Nc1c(-c2ccnc(CCc3ccc(F)cc3)n2)[nH]c2c1C(=O)NCC2] |
| 209 | 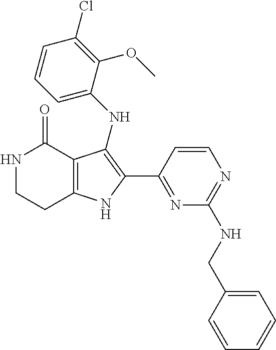 [SMILES: COc1c(Cl)cccc1Nc1c(-c2ccnc(NCc3ccccc3)n2)[nH]c2c1C(=O)NCC2] |
| 210 | 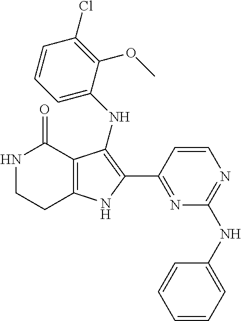 [SMILES: COc1c(Cl)cccc1Nc1c(-c2ccnc(Nc3ccccc3)n2)[nH]c2c1C(=O)NCC2] |
| 393a | 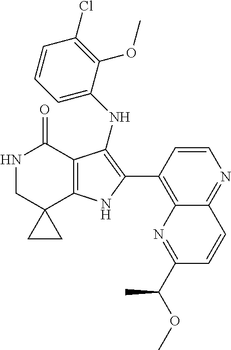 [SMILES: COc1c(Cl)cccc1Nc1c(-c2ccnc3ccc([C@H](C)OC)nc23)[nH]c2c1C(=O)NCC21CC1] |
| 393b | 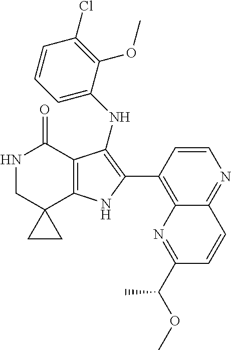 [SMILES: COc1c(Cl)cccc1Nc1c(-c2ccnc3ccc([C@@H](C)OC)nc23)[nH]c2c1C(=O)NCC21CC1] |
| 394 | 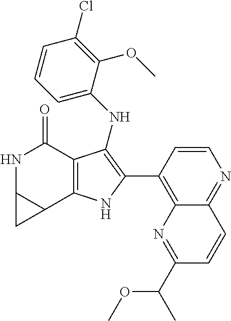 [SMILES: COc1c(Cl)cccc1Nc1c(-c2ccnc3ccc(C(C)OC)nc23)[nH]c2c1C(=O)NC1CC21] |
| 211 | 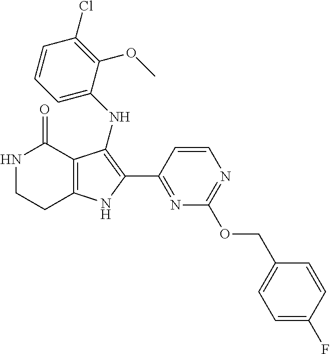 [SMILES: COc1c(Cl)cccc1Nc1c(-c2ccnc(OCc3ccc(F)cc3)n2)[nH]c2c1C(=O)NCC2] |
| 212 | 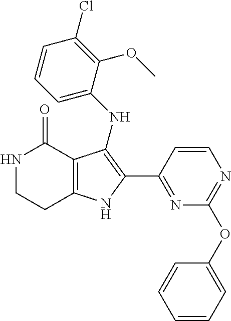 [SMILES: COc1c(Cl)cccc1Nc1c(-c2ccnc(Oc3ccccc3)n2)[nH]c2c1C(=O)NCC2] |
| 213 | 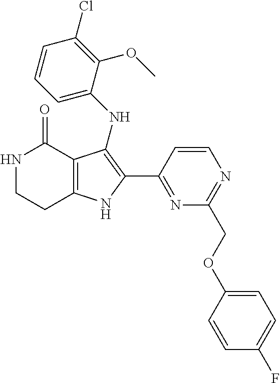 [SMILES: COc1c(Cl)cccc1Nc1c(-c2ccnc(COc3ccc(F)cc3)n2)[nH]c2c1C(=O)NCC2] |
| 394a | 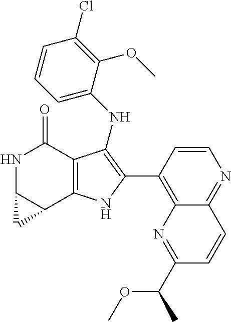 [SMILES: COc1c(Cl)cccc1Nc1c(-c2ccnc3ccc([C@@H](C)OC)nc23)[nH]c2c1C(=O)N[C@H]1C[C@@H]21] |
| 394b | 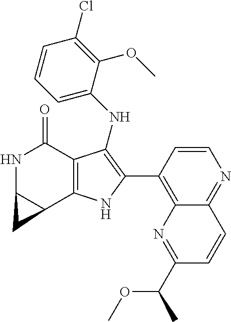 [SMILES: COc1c(Cl)cccc1Nc1c(-c2ccnc3ccc([C@@H](C)OC)nc23)[nH]c2c1C(=O)N[C@@H]1C[C@H]21] |
| 394c | 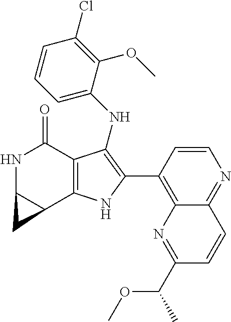 [SMILES: COc1c(Cl)cccc1Nc1c(-c2ccnc3ccc([C@H](C)OC)nc23)[nH]c2c1C(=O)N[C@@H]1C[C@H]21] |
| 214 | 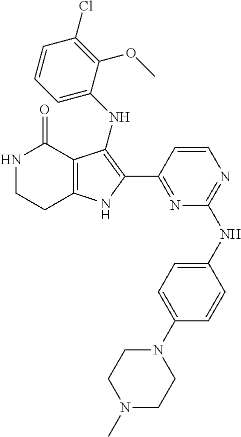 [SMILES: COc1c(Cl)cccc1Nc1c(-c2ccnc(Nc3ccc(N4CCN(C)CC4)cc3)n2)[nH]c2c1C(=O)NCC2] |
| 215 | 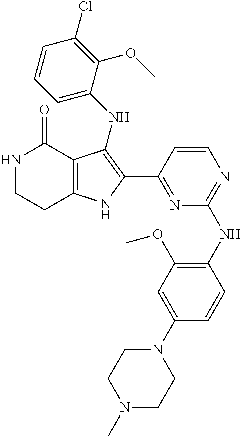 [SMILES: COc1cc(N2CCN(C)CC2)ccc1Nc1nccc(-c2[nH]c3c(c2Nc2cccc(Cl)c2OC)C(=O)NCC3)n1] |
| 216 | 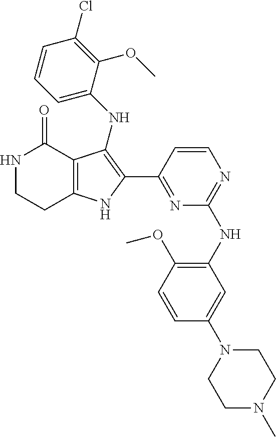 [SMILES: COc1ccc(N2CCN(C)CC2)cc1Nc1nccc(-c2[nH]c3c(c2Nc2cccc(Cl)c2OC)C(=O)NCC3)n1] |
| 394d | 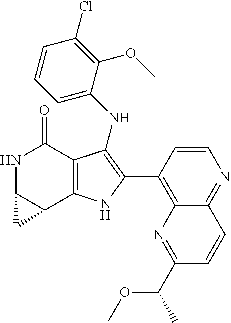 [SMILES: COc1c(Cl)cccc1Nc1c(-c2ccnc3ccc([C@H](C)OC)nc23)[nH]c2c1C(=O)N[C@H]1C[C@@H]21] |
| 395 | 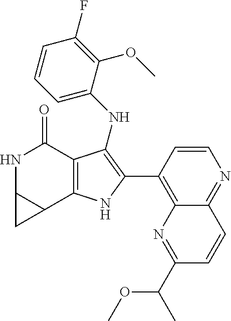 [SMILES: COc1c(F)cccc1Nc1c(-c2ccnc3ccc(C(C)OC)nc23)[nH]c2c1C(=O)NC1CC21] |
| 395a | 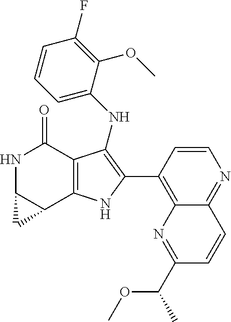 [SMILES: COc1c(F)cccc1Nc1c(-c2ccnc3ccc([C@H](C)OC)nc23)[nH]c2c1C(=O)N[C@H]1C[C@@H]21] |
| 217 | 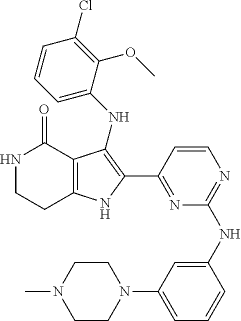 [SMILES: COc1c(Cl)cccc1Nc1c(-c2ccnc(Nc3cccc(N4CCN(C)CC4)c3)n2)[nH]c2c1C(=O)NCC2] |
| 218 | 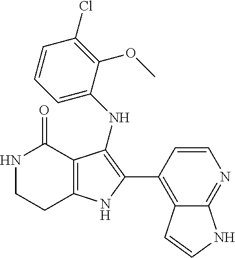 [SMILES: COc1c(Cl)cccc1Nc1c(-c2ccnc3[nH]ccc23)[nH]c2c1C(=O)NCC2] |
| 219 | 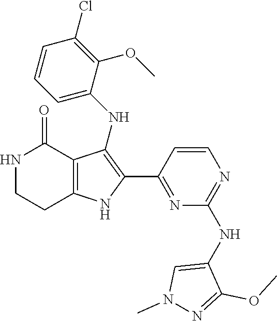 [SMILES: COc1nn(C)cc1Nc1nccc(-c2[nH]c3c(c2Nc2cccc(Cl)c2OC)C(=O)NCC3)n1] |
| 395b | 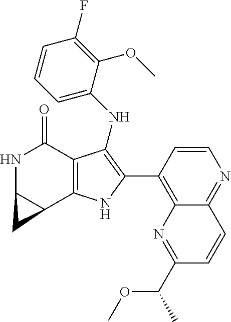 [SMILES: COc1c(F)cccc1Nc1c(-c2ccnc3ccc([C@H](C)OC)nc23)[nH]c2c1C(=O)N[C@@H]1C[C@H]21] |
| 395c | 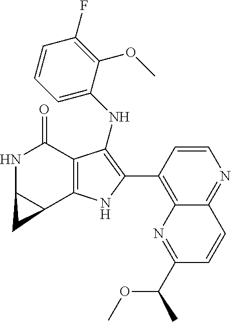 [SMILES: COc1c(F)cccc1Nc1c(-c2ccnc3ccc([C@@H](C)OC)nc23)[nH]c2c1C(=O)N[C@@H]1C[C@H]21] |
| 395d | 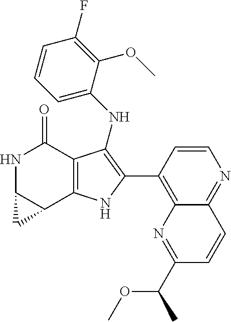 [SMILES: COc1c(F)cccc1Nc1c(-c2ccnc3ccc([C@@H](C)OC)nc23)[nH]c2c1C(=O)N[C@H]1C[C@@H]21] |
| 220 | 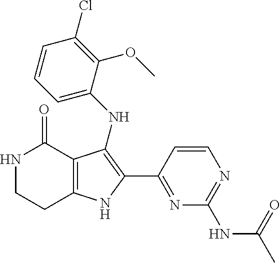 [SMILES: COc1c(Cl)cccc1Nc1c(-c2ccnc(NC(C)=O)n2)[nH]c2c1C(=O)NCC2] |
| 221 | 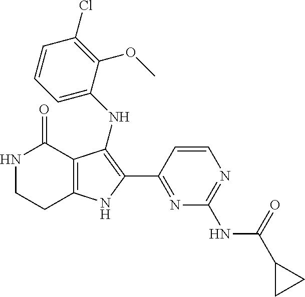 [SMILES: COc1c(Cl)cccc1Nc1c(-c2ccnc(NC(=O)C3CC3)n2)[nH]c2c1C(=O)NCC2] |
| 222 | 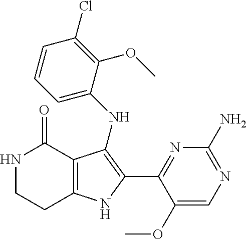 [SMILES: COc1cnc(N)nc1-c1[nH]c2c(c1Nc1cccc(Cl)c1OC)C(=O)NCC2] |
| 396 | 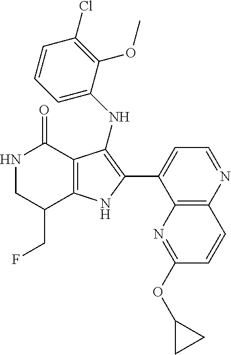 [SMILES: COc1c(Cl)cccc1Nc1c(-c2ccnc3ccc(OC4CC4)nc23)[nH]c2c1C(=O)NCC2CF] |
| 396a | 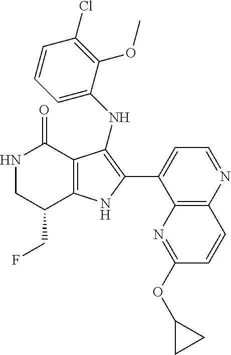 [SMILES: COc1c(Cl)cccc1Nc1c(-c2ccnc3ccc(OC4CC4)nc23)[nH]c2c1C(=O)NC[C@H]2CF] |
| 396b | 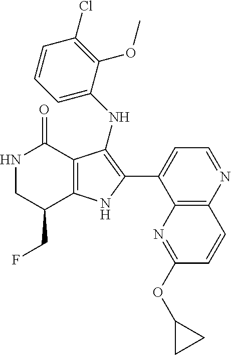 [SMILES: COc1c(Cl)cccc1Nc1c(-c2ccnc3ccc(OC4CC4)nc23)[nH]c2c1C(=O)NC[C@@H]2CF] |
| 223 | 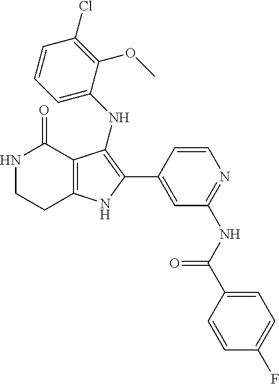 [SMILES: COc1c(Cl)cccc1Nc1c(-c2ccnc(NC(=O)c3ccc(F)cc3)c2)[nH]c2c1C(=O)NCC2] |
| 224 | 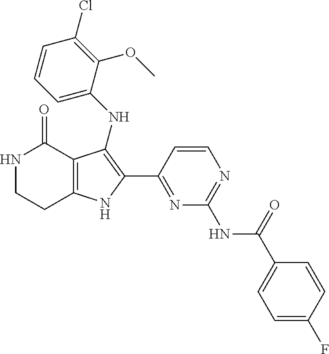 [SMILES: COc1c(Cl)cccc1Nc1c(-c2ccnc(NC(=O)c3ccc(F)cc3)n2)[nH]c2c1C(=O)NCC2] |
| 225 | 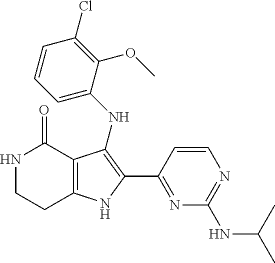 [SMILES: COc1c(Cl)cccc1Nc1c(-c2ccnc(NC(C)C)n2)[nH]c2c1C(=O)NCC2] |
| 397 | 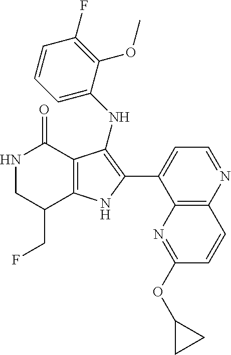 [SMILES: COc1c(F)cccc1Nc1c(-c2ccnc3ccc(OC4CC4)nc23)[nH]c2c1C(=O)NCC2CF] |
| 397a | 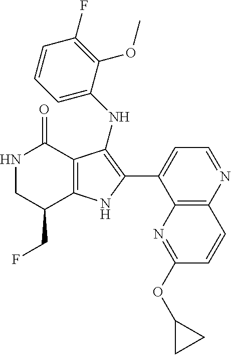 [SMILES: COc1c(F)cccc1Nc1c(-c2ccnc3ccc(OC4CC4)nc23)[nH]c2c1C(=O)NC[C@@H]2CF] |
| 397b | 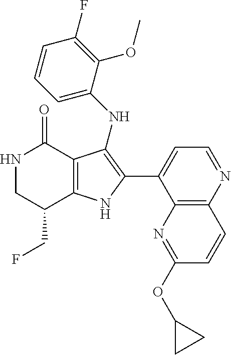 [SMILES: COc1c(F)cccc1Nc1c(-c2ccnc3ccc(OC4CC4)nc23)[nH]c2c1C(=O)NC[C@H]2CF] |
| 226 | 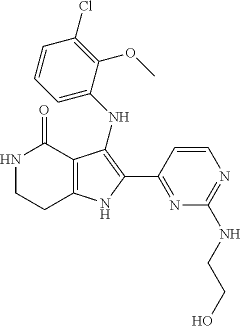 [SMILES: COc1c(Cl)cccc1Nc1c(-c2ccnc(NCCO)n2)[nH]c2c1C(=O)NCC2] |
| 227 | 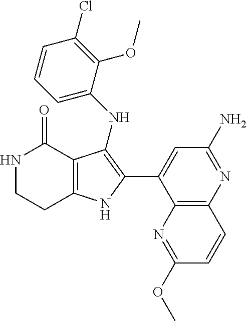 [SMILES: COc1ccc2nc(N)cc(-c3[nH]c4c(c3Nc3cccc(Cl)c3OC)C(=O)NCC4)c2n1] |
| 228 | 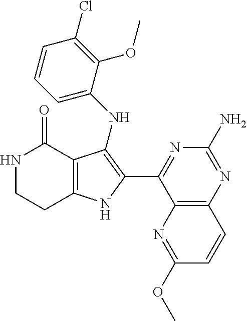 [SMILES: COc1ccc2nc(N)nc(-c3[nH]c4c(c3Nc3cccc(Cl)c3OC)C(=O)NCC4)c2n1] |
| 398 | 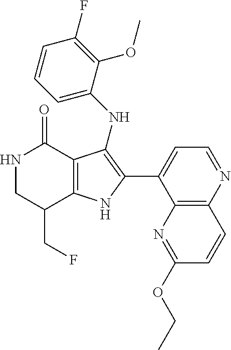 [SMILES: CCOc1ccc2nccc(-c3[nH]c4c(c3Nc3cccc(F)c3OC)C(=O)NCC4CF)c2n1] |
| 398a | 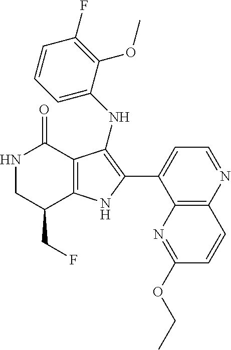 [SMILES: CCOc1ccc2nccc(-c3[nH]c4c(c3Nc3cccc(F)c3OC)C(=O)NC[C@@H]4CF)c2n1] |
| 398b | 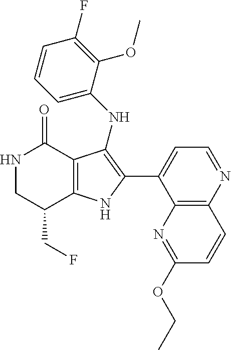 [SMILES: CCOc1ccc2nccc(-c3[nH]c4c(c3Nc3cccc(F)c3OC)C(=O)NC[C@H]4CF)c2n1] |
| 229 | 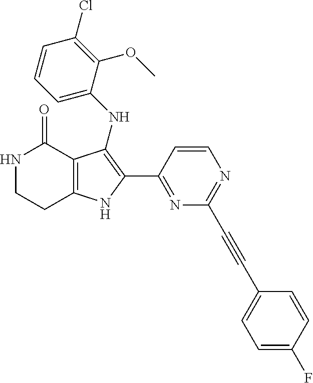 [SMILES: COc1c(Cl)cccc1Nc1c(-c2ccnc(C#Cc3ccc(F)cc3)n2)[nH]c2c1C(=O)NCC2] |
| 230 | 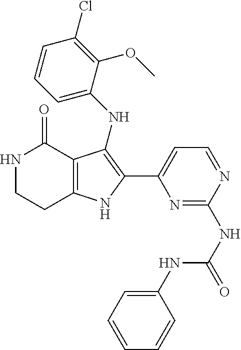 [SMILES: COc1c(Cl)cccc1Nc1c(-c2ccnc(NC(=O)Nc3ccccc3)n2)[nH]c2c1C(=O)NCC2] |
| 231 | 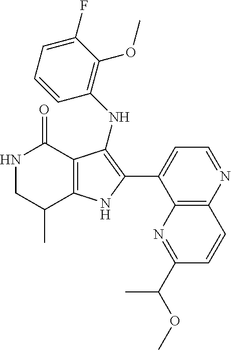 [SMILES: COc1c(F)cccc1Nc1c(-c2ccnc3ccc(C(C)OC)nc23)[nH]c2c1C(=O)NCC2C] |
| 399 | 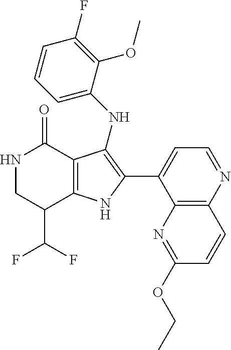 [SMILES: CCOc1ccc2nccc(-c3[nH]c4c(c3Nc3cccc(F)c3OC)C(=O)NCC4C(F)F)c2n1] |
| 399a | 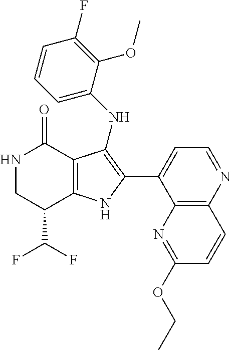 [SMILES: CCOc1ccc2nccc(-c3[nH]c4c(c3Nc3cccc(F)c3OC)C(=O)NC[C@H]4C(F)F)c2n1] |
| 399b | 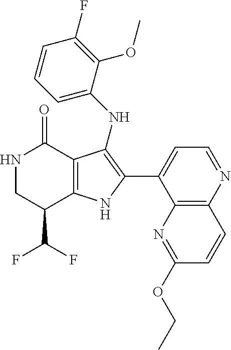 [SMILES: CCOc1ccc2nccc(-c3[nH]c4c(c3Nc3cccc(F)c3OC)C(=O)NC[C@@H]4C(F)F)c2n1] |
| 231a | 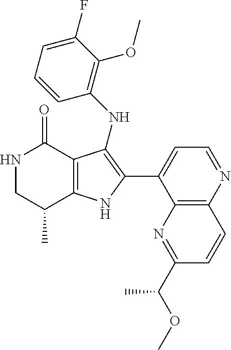 [SMILES: COc1c(F)cccc1Nc1c(-c2ccnc3ccc([C@@H](C)OC)nc23)[nH]c2c1C(=O)NC[C@H]2C] |
| 231b | 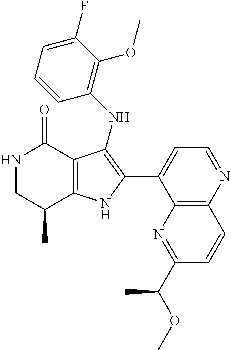 [SMILES: COc1c(F)cccc1Nc1c(-c2ccnc3ccc([C@H](C)OC)nc23)[nH]c2c1C(=O)NC[C@@H]2C] |
| 231c | 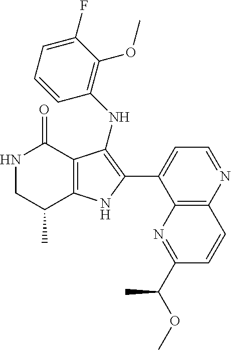 [SMILES: COc1c(F)cccc1Nc1c(-c2ccnc3ccc([C@H](C)OC)nc23)[nH]c2c1C(=O)NC[C@H]2C] |
| 400 | 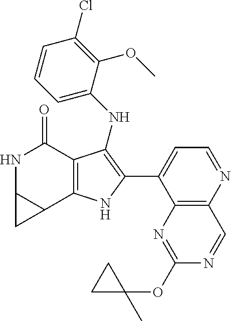 [SMILES: COc1c(Cl)cccc1Nc1c(-c2ccnc3cnc(OC4(C)CC4)nc23)[nH]c2c1C(=O)NC1CC21] |
| 400a | 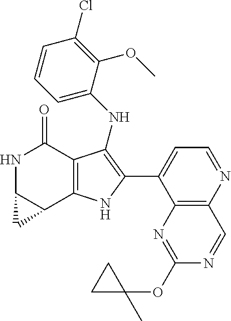 [SMILES: COc1c(Cl)cccc1Nc1c(-c2ccnc3cnc(OC4(C)CC4)nc23)[nH]c2c1C(=O)N[C@H]1C[C@@H]21] |
| 400b | 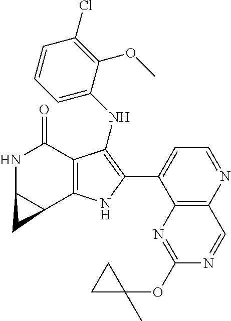 [SMILES: COc1c(Cl)cccc1Nc1c(-c2ccnc3cnc(OC4(C)CC4)nc23)[nH]c2c1C(=O)N[C@@H]1C[C@H]21] |
| 231d | 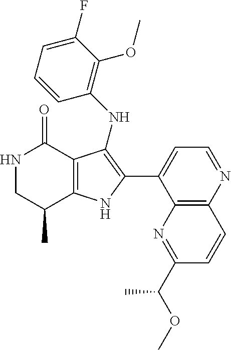 [SMILES: COc1c(F)cccc1Nc1c(-c2ccnc3ccc([C@@H](C)OC)nc23)[nH]c2c1C(=O)NC[C@@H]2C] |
| 232 | 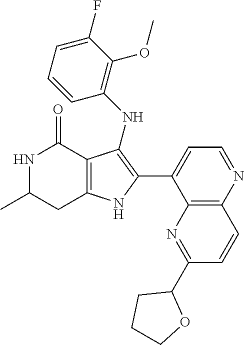 [SMILES: COc1c(F)cccc1Nc1c(-c2ccnc3ccc(C4CCCO4)nc23)[nH]c2c1C(=O)NC(C)C2] |
| 232a | 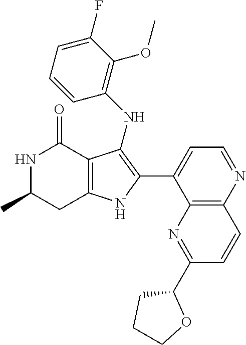 [SMILES: COc1c(F)cccc1Nc1c(-c2ccnc3ccc([C@H]4CCCO4)nc23)[nH]c2c1C(=O)N[C@H](C)C2] |
| 401 | 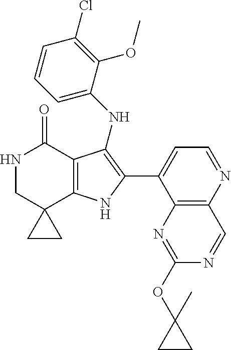 [SMILES: COc1c(Cl)cccc1Nc1c(-c2ccnc3cnc(OC4(C)CC4)nc23)[nH]c2c1C(=O)NCC21CC1] |
| 402 | 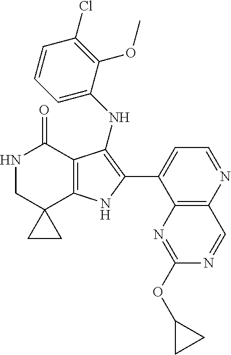 [SMILES: COc1c(Cl)cccc1Nc1c(-c2ccnc3cnc(OC4CC4)nc23)[nH]c2c1C(=O)NCC21CC1] |
| 403 | 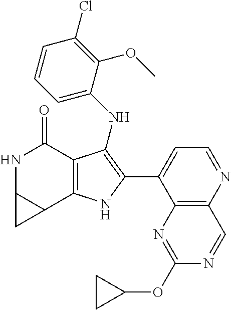 [SMILES: COc1c(Cl)cccc1Nc1c(-c2ccnc3cnc(OC4CC4)nc23)[nH]c2c1C(=O)NC1CC21] |
| 232b | 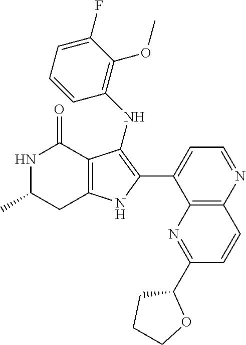 [SMILES: COc1c(F)cccc1Nc1c(-c2ccnc3ccc([C@H]4CCCO4)nc23)[nH]c2c1C(=O)N[C@@H](C)C2] |
| 232c | 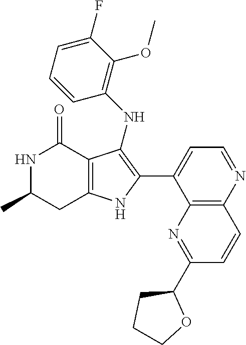 [SMILES: COc1c(F)cccc1Nc1c(-c2ccnc3ccc([C@@H]4CCCO4)nc23)[nH]c2c1C(=O)N[C@H](C)C2] |
| 232d | 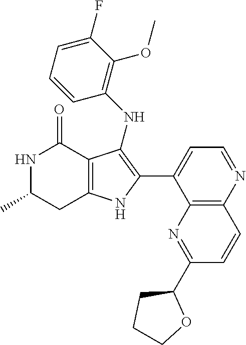 [SMILES: COc1c(F)cccc1Nc1c(-c2ccnc3ccc([C@@H]4CCCO4)nc23)[nH]c2c1C(=O)N[C@@H](C)C2] |
| 403a | 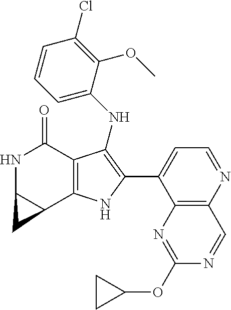 [SMILES: COc1c(Cl)cccc1Nc1c(-c2ccnc3cnc(OC4CC4)nc23)[nH]c2c1C(=O)N[C@@H]1C[C@H]21] |
| 403b | 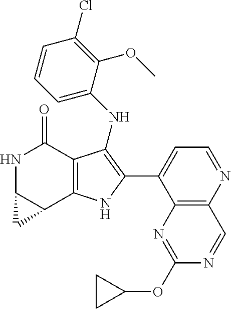 [SMILES: COc1c(Cl)cccc1Nc1c(-c2ccnc3cnc(OC4CC4)nc23)[nH]c2c1C(=O)N[C@H]1C[C@@H]21] |
| 404 | 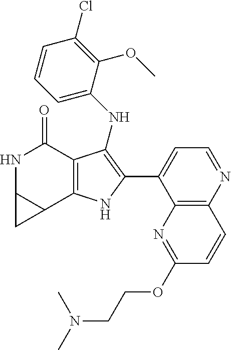 [SMILES: COc1c(Cl)cccc1Nc1c(-c2ccnc3ccc(OCCN(C)C)nc23)[nH]c2c1C(=O)NC1CC21] |
| 233 | 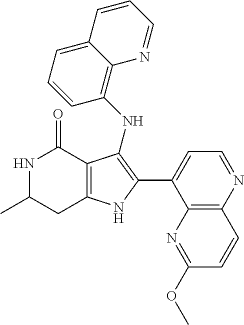 [SMILES: COc1ccc2nccc(-c3[nH]c4c(c3Nc3cccc5cccnc35)C(=O)NC(C)C4)c2n1] |
| 233a | 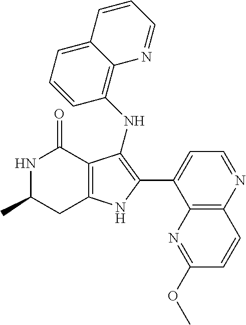 [SMILES: COc1ccc2nccc(-c3[nH]c4c(c3Nc3cccc5cccnc35)C(=O)N[C@H](C)C4)c2n1] |
| 233b | 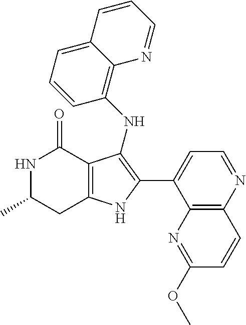 [SMILES: COc1ccc2nccc(-c3[nH]c4c(c3Nc3cccc5cccnc35)C(=O)N[C@@H](C)C4)c2n1] |
| 404a | 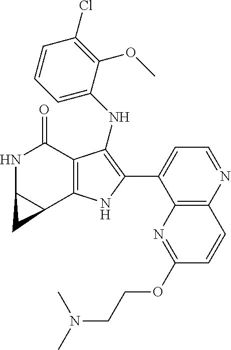 [SMILES: COc1c(Cl)cccc1Nc1c(-c2ccnc3ccc(OCCN(C)C)nc23)[nH]c2c1C(=O)N[C@@H]1C[C@H]21] |
| 404b | 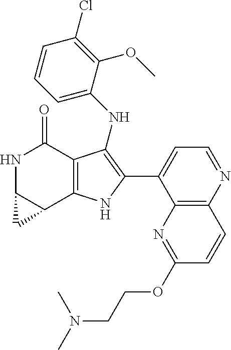 [SMILES: COc1c(Cl)cccc1Nc1c(-c2ccnc3ccc(OCCN(C)C)nc23)[nH]c2c1C(=O)N[C@H]1C[C@@H]21] |
| 405 | 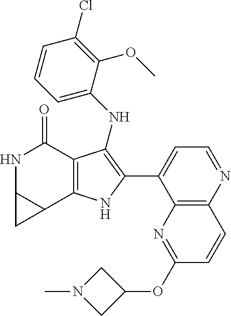 [SMILES: COc1c(Cl)cccc1Nc1c(-c2ccnc3ccc(OC4CN(C)C4)nc23)[nH]c2c1C(=O)NC1CC21] |
| 234 | 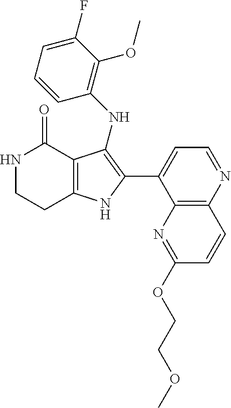 [SMILES: COCCOc1ccc2nccc(-c3[nH]c4c(c3Nc3cccc(F)c3OC)C(=O)NCC4)c2n1] |
| 235 | 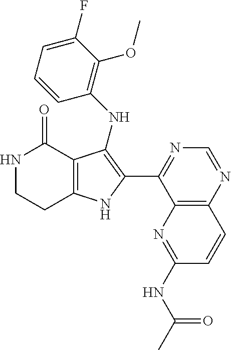 [SMILES: COc1c(F)cccc1Nc1c(-c2ncnc3ccc(NC(C)=O)nc23)[nH]c2c1C(=O)NCC2] |
| 236 | 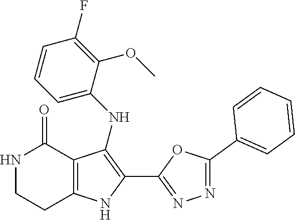 [SMILES: COc1c(F)cccc1Nc1c(-c2nnc(-c3ccccc3)o2)[nH]c2c1C(=O)NCC2] |
| 405a | 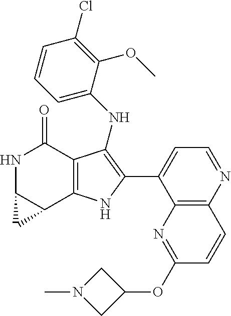 [SMILES: COc1c(Cl)cccc1Nc1c(-c2ccnc3ccc(OC4CN(C)C4)nc23)[nH]c2c1C(=O)N[C@H]1C[C@@H]21] |
| 405b | 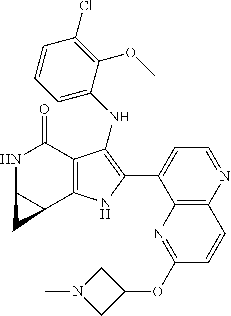 [SMILES: COc1c(Cl)cccc1Nc1c(-c2ccnc3ccc(OC4CN(C)C4)nc23)[nH]c2c1C(=O)N[C@@H]1C[C@H]21] |
| 406 | 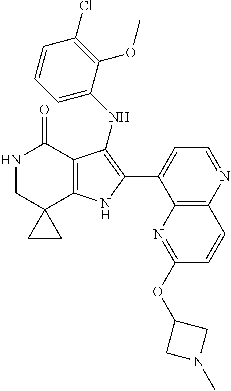 [SMILES: COc1c(Cl)cccc1Nc1c(-c2ccnc3ccc(OC4CN(C)C4)nc23)[nH]c2c1C(=O)NCC21CC1] |
| 237 | 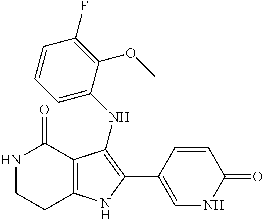 [SMILES: COc1c(F)cccc1Nc1c(-c2ccc(=O)[nH]c2)[nH]c2c1C(=O)NCC2] |
| 238 | 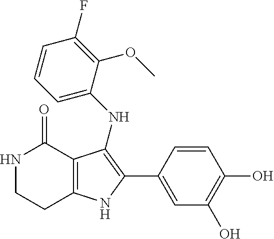 [SMILES: COc1c(F)cccc1Nc1c(-c2ccc(O)c(O)c2)[nH]c2c1C(=O)NCC2] |
| 239 | 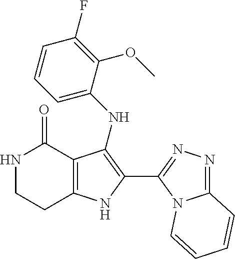 [SMILES: COc1c(F)cccc1Nc1c(-c2nnc3ccccn23)[nH]c2c1C(=O)NCC2] |
| 240 | 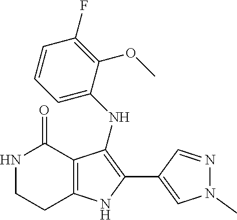 [SMILES: COc1c(F)cccc1Nc1c(-c2cnn(C)c2)[nH]c2c1C(=O)NCC2] |
| 407 | 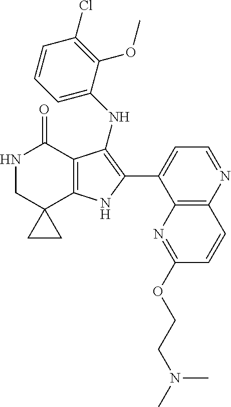 [SMILES: COc1c(Cl)cccc1Nc1c(-c2ccnc3ccc(OCCN(C)C)nc23)[nH]c2c1C(=O)NCC21CC1] |
| 408 | 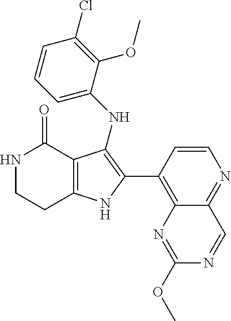 [SMILES: COc1ncc2nccc(-c3[nH]c4c(c3Nc3cccc(Cl)c3OC)C(=O)NCC4)c2n1] |
| 409 | 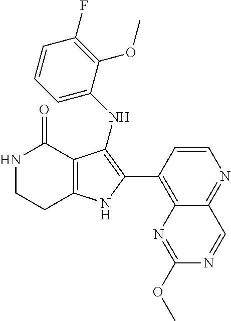 [SMILES: COc1ncc2nccc(-c3[nH]c4c(c3Nc3cccc(F)c3OC)C(=O)NCC4)c2n1] |
| 410 | 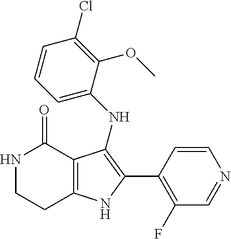 [SMILES: COc1c(Cl)cccc1Nc1c(-c2ccncc2F)[nH]c2c1C(=O)NCC2] |
| 241 | 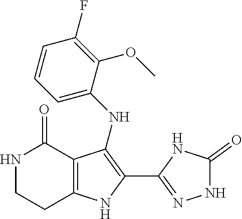 [SMILES: COc1c(F)cccc1Nc1c(-c2n[nH]c(=O)[nH]2)[nH]c2c1C(=O)NCC2] |
| 242 | 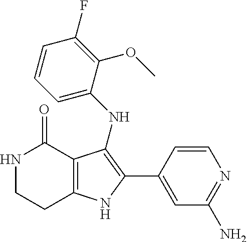 [SMILES: COc1c(F)cccc1Nc1c(-c2ccnc(N)c2)[nH]c2c1C(=O)NCC2] |
| 243 | 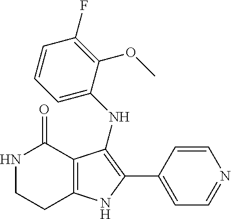 [SMILES: COc1c(F)cccc1Nc1c(-c2ccncc2)[nH]c2c1C(=O)NCC2] |
| 244 | 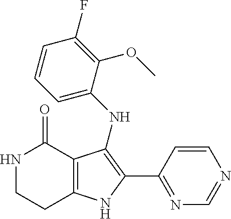 [SMILES: COc1c(F)cccc1Nc1c(-c2ccncn2)[nH]c2c1C(=O)NCC2] |
| 411 | 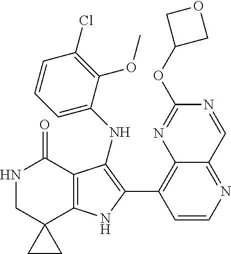 [SMILES: COc1c(Cl)cccc1Nc1c(-c2ccnc3cnc(OC4COC4)nc23)[nH]c2c1C(=O)NCC21CC1] |
| 412 | 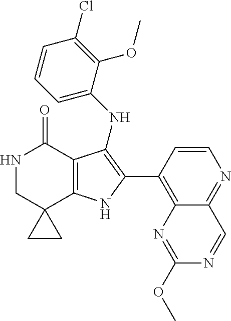 [SMILES: COc1ncc2nccc(-c3[nH]c4c(c3Nc3cccc(Cl)c3OC)C(=O)NCC43CC3)c2n1] |
| 413 | 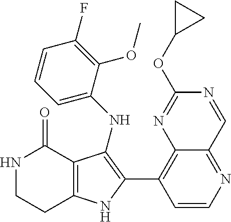 [SMILES: COc1c(F)cccc1Nc1c(-c2ccnc3cnc(OC4CC4)nc23)[nH]c2c1C(=O)NCC2] |
| 414 | 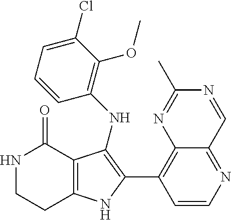 [SMILES: COc1c(Cl)cccc1Nc1c(-c2ccnc3cnc(C)nc23)[nH]c2c1C(=O)NCC2] |
| 245 | 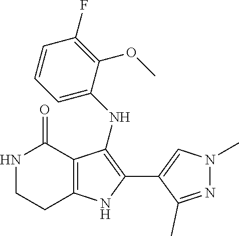 [SMILES: COc1c(F)cccc1Nc1c(-c2cn(C)nc2C)[nH]c2c1C(=O)NCC2] |
| 246 | 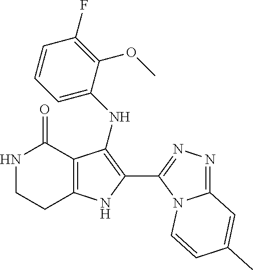 [SMILES: COc1c(F)cccc1Nc1c(-c2nnc3cc(C)ccn23)[nH]c2c1C(=O)NCC2] |
| 247 | 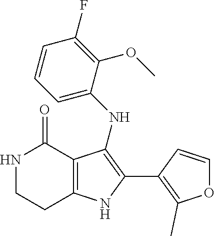 [SMILES: COc1c(F)cccc1Nc1c(-c2ccoc2C)[nH]c2c1C(=O)NCC2] |
| 248 | 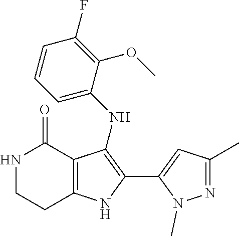 [SMILES: COc1c(F)cccc1Nc1c(-c2cc(C)nn2C)[nH]c2c1C(=O)NCC2] |
| 415 | 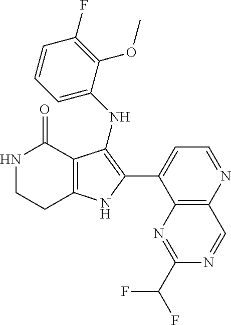 [SMILES: COc1c(F)cccc1Nc1c(-c2ccnc3cnc(C(F)F)nc23)[nH]c2c1C(=O)NCC2] |
| 416 | 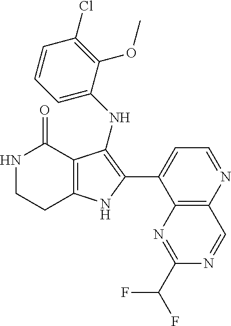 [SMILES: COc1c(Cl)cccc1Nc1c(-c2ccnc3cnc(C(F)F)nc23)[nH]c2c1C(=O)NCC2] |
| 417 | 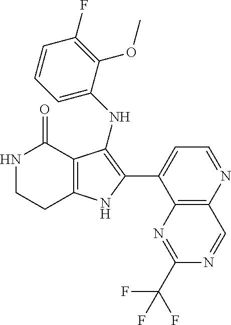 [SMILES: COc1c(F)cccc1Nc1c(-c2ccnc3cnc(C(F)(F)F)nc23)[nH]c2c1C(=O)NCC2] |
| 418 | 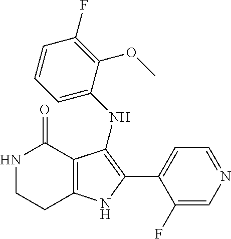 [SMILES: COc1c(F)cccc1Nc1c(-c2ccncc2F)[nH]c2c1C(=O)NCC2] |
| 249 | 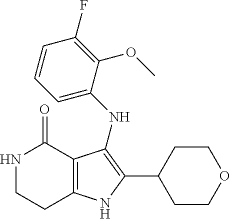 [SMILES: COc1c(F)cccc1Nc1c(C2CCOCC2)[nH]c2c1C(=O)NCC2] |
| 250 | 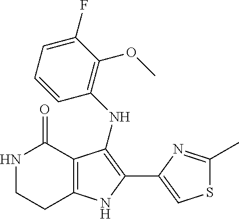 [SMILES: COc1c(F)cccc1Nc1c(-c2csc(C)n2)[nH]c2c1C(=O)NCC2] |
| 251 | 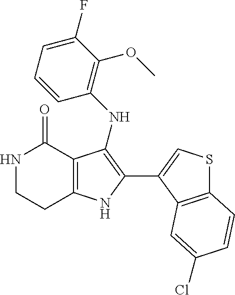 [SMILES: COc1c(F)cccc1Nc1c(-c2csc3ccc(Cl)cc23)[nH]c2c1C(=O)NCC2] |
| 252 | 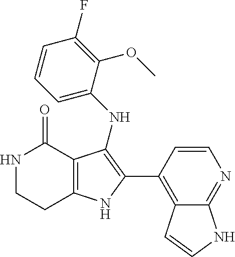 [SMILES: COc1c(F)cccc1Nc1c(-c2ccnc3[nH]ccc23)[nH]c2c1C(=O)NCC2] |
| 419 | 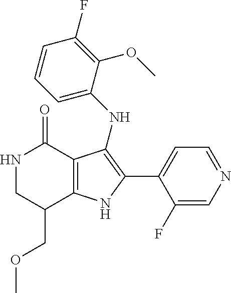 [SMILES: COCC1CNC(=O)c2c1[nH]c(-c1ccncc1F)c2Nc1cccc(F)c1OC] |
| 419a | 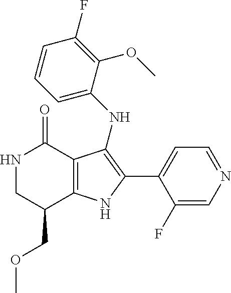 [SMILES: COC[C@H]1CNC(=O)c2c1[nH]c(-c1ccncc1F)c2Nc1cccc(F)c1OC] |
| 419b | 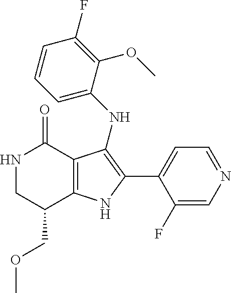 [SMILES: COC[C@@H]1CNC(=O)c2c1[nH]c(-c1ccncc1F)c2Nc1cccc(F)c1OC] |
| 420 | 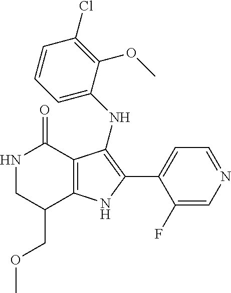 [SMILES: COCC1CNC(=O)c2c1[nH]c(-c1ccncc1F)c2Nc1cccc(Cl)c1OC] |
| 253 | 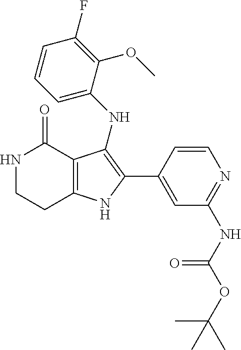 [SMILES: COc1c(F)cccc1Nc1c(-c2ccnc(NC(=O)OC(C)(C)C)c2)[nH]c2c1C(=O)NCC2] |
| 254 | 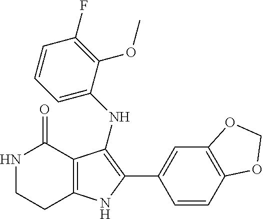 [SMILES: COc1c(F)cccc1Nc1c(-c2ccc3c(c2)OCO3)[nH]c2c1C(=O)NCC2] |
| 255 | 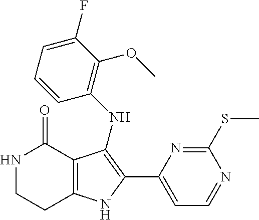 [SMILES: COc1c(F)cccc1Nc1c(-c2ccnc(SC)n2)[nH]c2c1C(=O)NCC2] |
| 256 | 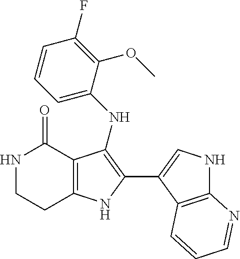 [SMILES: COc1c(F)cccc1Nc1c(-c2c[nH]c3ncccc23)[nH]c2c1C(=O)NCC2] |
| 420a | 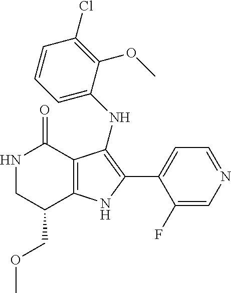 [SMILES: COC[C@@H]1CNC(=O)c2c1[nH]c(-c1ccncc1F)c2Nc1cccc(Cl)c1OC] |
| 420b | 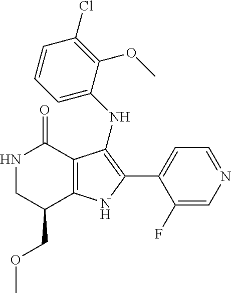 [SMILES: COC[C@H]1CNC(=O)c2c1[nH]c(-c1ccncc1F)c2Nc1cccc(Cl)c1OC] |
| 421 | 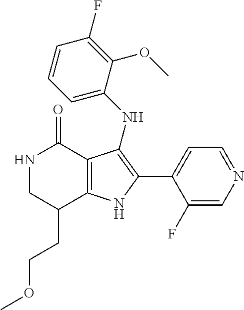 [SMILES: COCCC1CNC(=O)c2c1[nH]c(-c1ccncc1F)c2Nc1cccc(F)c1OC] |
| 421a | 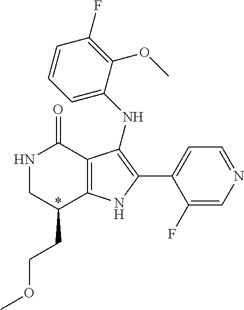 [SMILES: COCC[C@H]1CNC(=O)c2c1[nH]c(-c1ccncc1F)c2Nc1cccc(F)c1OC] |
| 257 | 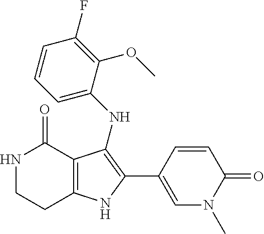 [SMILES: COc1c(F)cccc1Nc1c(-c2ccc(=O)n(C)c2)[nH]c2c1C(=O)NCC2] |
| 258 | 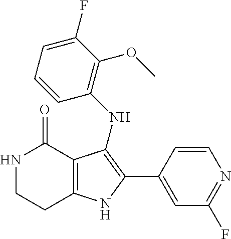 [SMILES: COc1c(F)cccc1Nc1c(-c2ccnc(F)c2)[nH]c2c1C(=O)NCC2] |
| 259 | 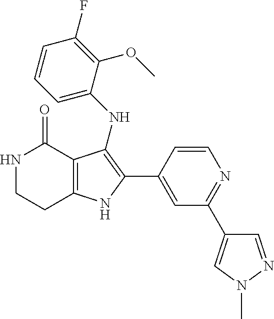 [SMILES: COc1c(F)cccc1Nc1c(-c2ccnc(-c3cnn(C)c3)c2)[nH]c2c1C(=O)NCC2] |
| 260 | 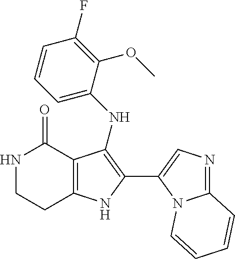 [SMILES: COc1c(F)cccc1Nc1c(-c2cnc3ccccn23)[nH]c2c1C(=O)NCC2] |
| 421b | 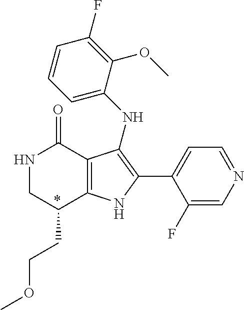 [SMILES: COCC[C@@H]1CNC(=O)c2c1[nH]c(-c1ccncc1F)c2Nc1cccc(F)c1OC] |
| 422 | 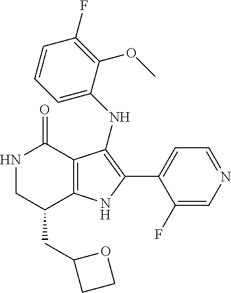 [SMILES: COc1c(F)cccc1Nc1c(-c2ccncc2F)[nH]c2c1C(=O)NC[C@H]2CC1CCO1] |
| 422a | 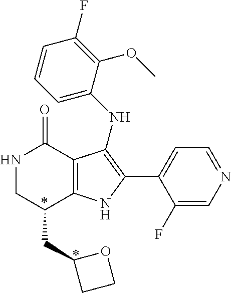 [SMILES: COc1c(F)cccc1Nc1c(-c2ccncc2F)[nH]c2c1C(=O)NC[C@H]2C[C@@H]1CCO1] |
| 422b | 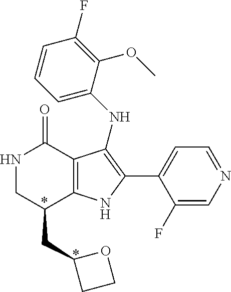 [SMILES: COc1c(F)cccc1Nc1c(-c2ccncc2F)[nH]c2c1C(=O)NC[C@@H]2C[C@@H]1CCO1] |
| 261 | 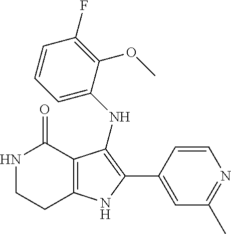 [SMILES: COc1c(F)cccc1Nc1c(-c2ccnc(C)c2)[nH]c2c1C(=O)NCC2] |
| 262 | 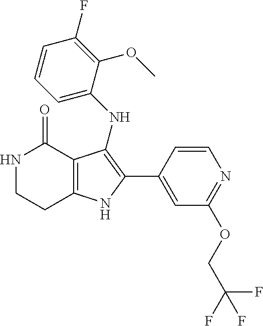 [SMILES: COc1c(F)cccc1Nc1c(-c2ccnc(OCC(F)(F)F)c2)[nH]c2c1C(=O)NCC2] |
| 263 | 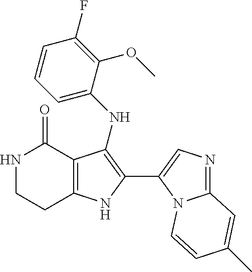 [SMILES: COc1c(F)cccc1Nc1c(-c2cnc3cc(C)ccn23)[nH]c2c1C(=O)NCC2] |
| 264 | 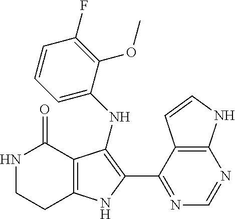 [SMILES: COc1c(F)cccc1Nc1c(-c2ncnc3[nH]ccc23)[nH]c2c1C(=O)NCC2] |
| 422c | 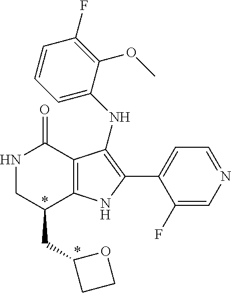 [SMILES: COc1c(F)cccc1Nc1c(-c2ccncc2F)[nH]c2c1C(=O)NC[C@@H]2C[C@H]1CCO1] |
| 422d | 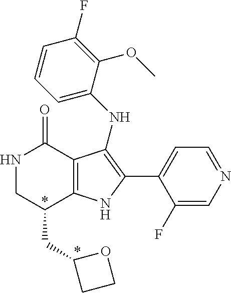 [SMILES: COc1c(F)cccc1Nc1c(-c2ccncc2F)[nH]c2c1C(=O)NC[C@H]2C[C@H]1CCO1] |
| 423 | 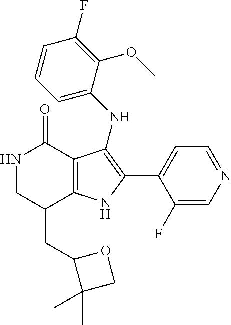 [SMILES: COc1c(F)cccc1Nc1c(-c2ccncc2F)[nH]c2c1C(=O)NCC2CC1OCC1(C)C] |
| 423a | 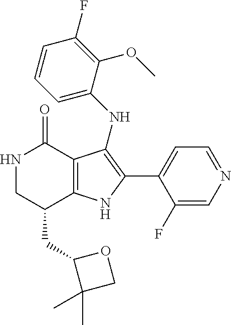 [SMILES: COc1c(F)cccc1Nc1c(-c2ccncc2F)[nH]c2c1C(=O)NC[C@H]2C[C@@H]1OCC1(C)C] |
| 265 | 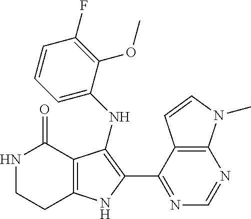 [SMILES: COc1c(F)cccc1Nc1c(-c2ncnc3c2ccn3C)[nH]c2c1C(=O)NCC2] |
| 266 | 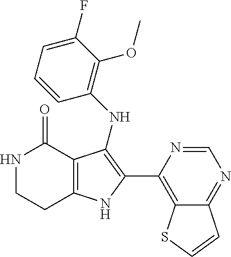 [SMILES: COc1c(F)cccc1Nc1c(-c2ncnc3ccsc23)[nH]c2c1C(=O)NCC2] |
| 267 | 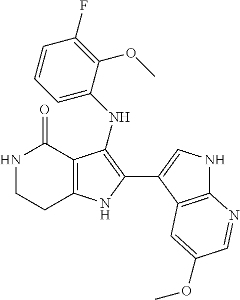 [SMILES: COc1cnc2[nH]cc(-c3[nH]c4c(c3Nc3cccc(F)c3OC)C(=O)NCC4)c2c1] |
| 268 | 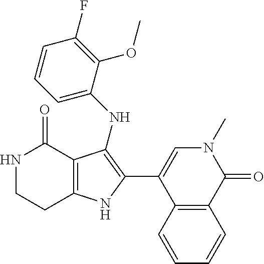 [SMILES: COc1c(F)cccc1Nc1c(-c2cn(C)c(=O)c3ccccc23)[nH]c2c1C(=O)NCC2] |
| 423b | 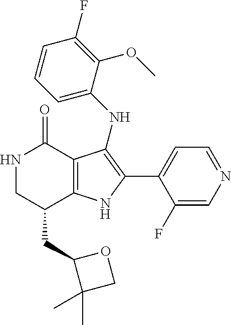 [SMILES: COc1c(F)cccc1Nc1c(-c2ccncc2F)[nH]c2c1C(=O)NC[C@H]2C[C@H]1OCC1(C)C] |
| 423c | 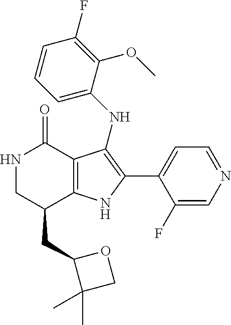 [SMILES: COc1c(F)cccc1Nc1c(-c2ccncc2F)[nH]c2c1C(=O)NC[C@@H]2C[C@H]1OCC1(C)C] |
| 423d | 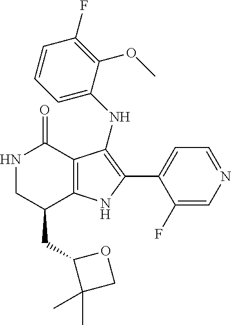 [SMILES: COc1c(F)cccc1Nc1c(-c2ccncc2F)[nH]c2c1C(=O)NC[C@@H]2C[C@@H]1OCC1(C)C] |
| 424 | 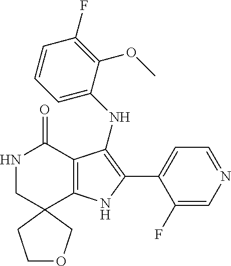 [SMILES: COc1c(F)cccc1Nc1c(-c2ccncc2F)[nH]c2c1C(=O)NCC21CCOC1] |
| 269 | 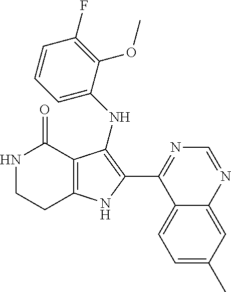 [SMILES: COc1c(F)cccc1Nc1c(-c2ncnc3cc(C)ccc23)[nH]c2c1C(=O)NCC2] |
| 270 | 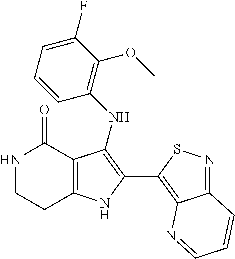 [SMILES: COc1c(F)cccc1Nc1c(-c2snc3cccnc23)[nH]c2c1C(=O)NCC2] |
| 271 | 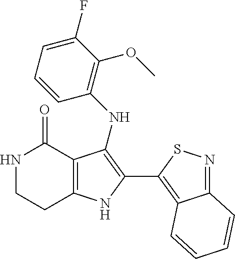 [SMILES: COc1c(F)cccc1Nc1c(-c2snc3ccccc23)[nH]c2c1C(=O)NCC2] |
| 272 | 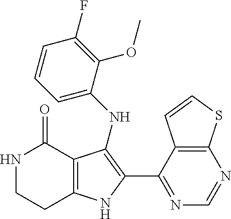 [SMILES: COc1c(F)cccc1Nc1c(-c2ncnc3sccc23)[nH]c2c1C(=O)NCC2] |
| 424a | 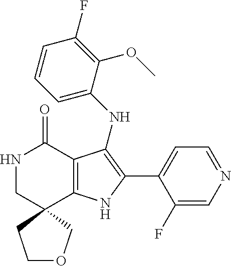 [SMILES: COc1c(F)cccc1Nc1c(-c2ccncc2F)[nH]c2c1C(=O)NC[C@]21CCOC1] |
| 424b | 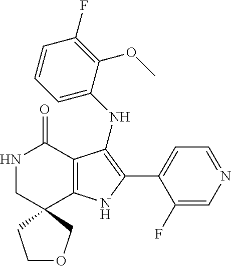 [SMILES: COc1c(F)cccc1Nc1c(-c2ccncc2F)[nH]c2c1C(=O)NC[C@@]21CCOC1] |
| 425 | 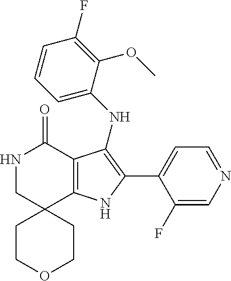 [SMILES: COc1c(F)cccc1Nc1c(-c2ccncc2F)[nH]c2c1C(=O)NCC21CCOCC1] |
| 426 | 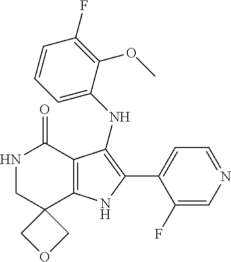 [SMILES: COc1c(F)cccc1Nc1c(-c2ccncc2F)[nH]c2c1C(=O)NCC21COC1] |
| 273 | 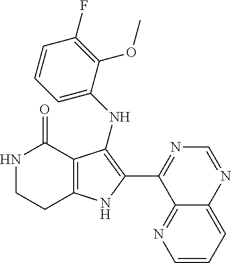 [SMILES: COc1c(F)cccc1Nc1c(-c2ncnc3cccnc23)[nH]c2c1C(=O)NCC2] |
| 274 | 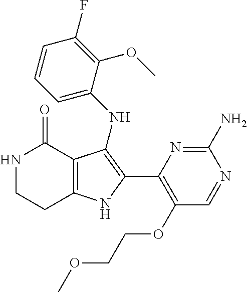 [SMILES: COCCOc1cnc(N)nc1-c1[nH]c2c(c1Nc1cccc(F)c1OC)C(=O)NCC2] |
| 275 | 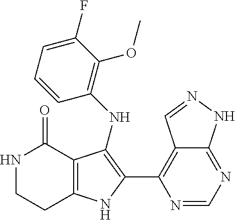 [SMILES: COc1c(F)cccc1Nc1c(-c2ncnc3[nH]ncc23)[nH]c2c1C(=O)NCC2] |
| 276 | 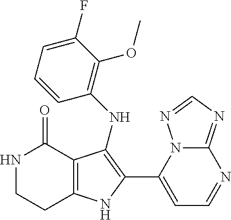 [SMILES: COc1c(F)cccc1Nc1c(-c2ccnc3ncnn23)[nH]c2c1C(=O)NCC2] |
| 427 | 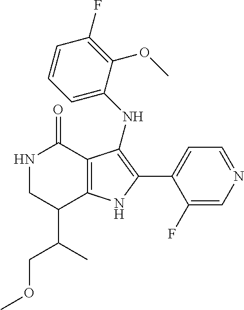 [SMILES: COCC(C)C1CNC(=O)c2c1[nH]c(-c1ccncc1F)c2Nc1cccc(F)c1OC] |
| 427a | 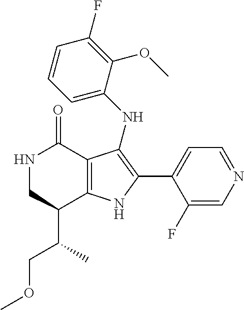 [SMILES: COC[C@@H](C)[C@H]1CNC(=O)c2c1[nH]c(-c1ccncc1F)c2Nc1cccc(F)c1OC] |
| 427b | 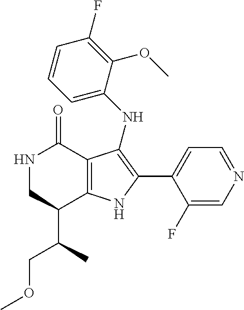 [SMILES: COC[C@H](C)[C@H]1CNC(=O)c2c1[nH]c(-c1ccncc1F)c2Nc1cccc(F)c1OC] |
| 427c | 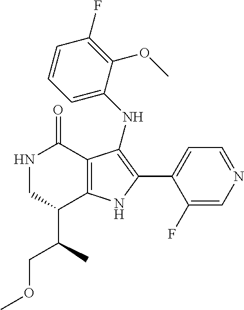 [SMILES: COC[C@H](C)[C@@H]1CNC(=O)c2c1[nH]c(-c1ccncc1F)c2Nc1cccc(F)c1OC] |
| 277 | 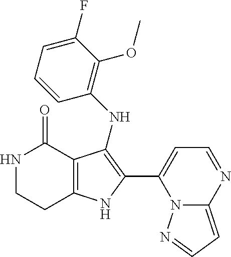 [SMILES: COc1c(F)cccc1Nc1c(-c2ccnc3ccnn23)[nH]c2c1C(=O)NCC2] |
| 278 | 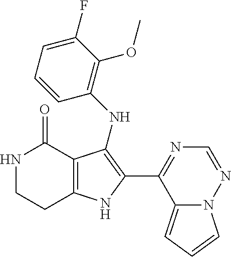 [SMILES: COc1c(F)cccc1Nc1c(-c2ncnn3cccc23)[nH]c2c1C(=O)NCC2] |
| 279 | 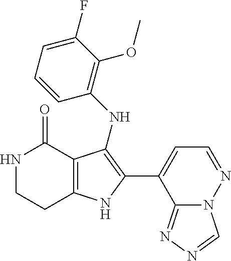 [SMILES: COc1c(F)cccc1Nc1c(-c2ccnn3cnnc23)[nH]c2c1C(=O)NCC2] |
| 280 | 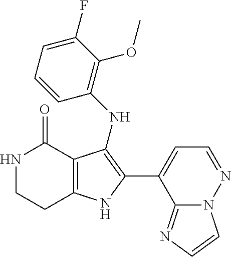 [SMILES: COc1c(F)cccc1Nc1c(-c2ccnn3ccnc23)[nH]c2c1C(=O)NCC2] |
| 427d | 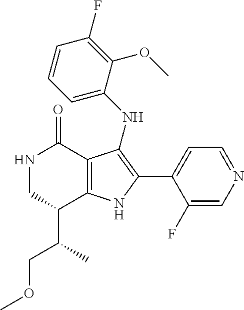 [SMILES: COC[C@@H](C)[C@@H]1CNC(=O)c2c1[nH]c(-c1ccncc1F)c2Nc1cccc(F)c1OC] |
| 428 | 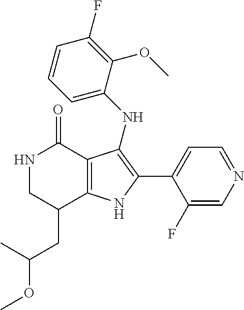 [SMILES: COc1c(F)cccc1Nc1c(-c2ccncc2F)[nH]c2c1C(=O)NCC2CC(C)OC] |
| 428a | 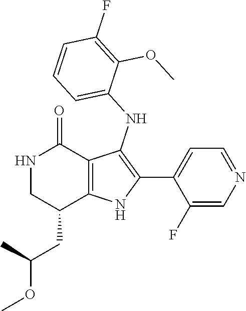 [SMILES: COc1c(F)cccc1Nc1c(-c2ccncc2F)[nH]c2c1C(=O)NC[C@H]2C[C@H](C)OC] |
| 428b | 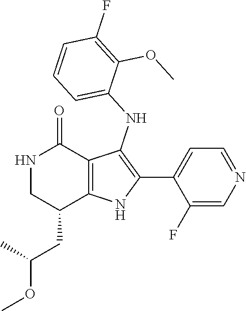 [SMILES: COc1c(F)cccc1Nc1c(-c2ccncc2F)[nH]c2c1C(=O)NC[C@H]2C[C@@H](C)OC] |
| 281 | 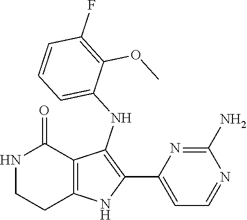 [SMILES: COc1c(F)cccc1Nc1c(-c2ccnc(N)n2)[nH]c2c1C(=O)NCC2] |
| 282 | 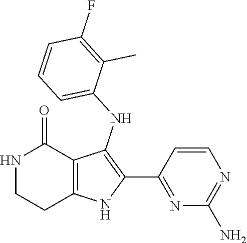 [SMILES: Cc1c(F)cccc1Nc1c(-c2ccnc(N)n2)[nH]c2c1C(=O)NCC2] |
| 283 | 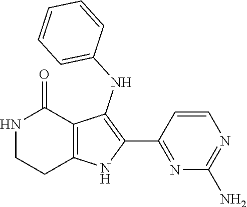 [SMILES: Nc1nccc(-c2[nH]c3c(c2Nc2ccccc2)C(=O)NCC3)n1] |
| 428c | 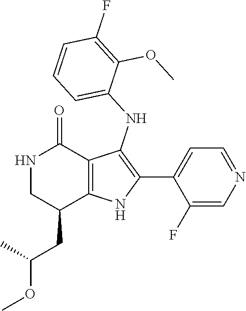 [SMILES: COc1c(F)cccc1Nc1c(-c2ccncc2F)[nH]c2c1C(=O)NC[C@@H]2C[C@@H](C)OC] |
| 428d | 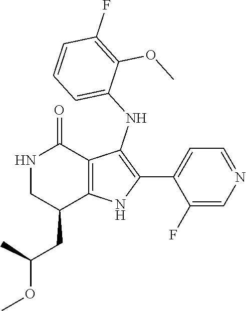 [SMILES: COc1c(F)cccc1Nc1c(-c2ccncc2F)[nH]c2c1C(=O)NC[C@@H]2C[C@H](C)OC] |
| | 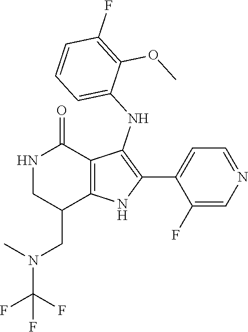 [SMILES: COc1c(F)cccc1Nc1c(-c2ccncc2F)[nH]c2c1C(=O)NCC2CN(C)C(F)(F)F] |
| 284 | 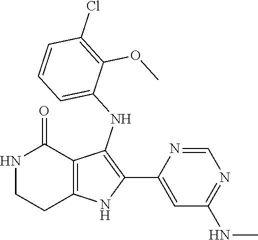 [SMILES: CNc1cc(-c2[nH]c3c(c2Nc2cccc(Cl)c2OC)C(=O)NCC3)ncn1] |
| 285 | 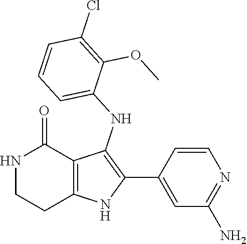 [SMILES: COc1c(Cl)cccc1Nc1c(-c2ccnc(N)c2)[nH]c2c1C(=O)NCC2] |
| 286 | 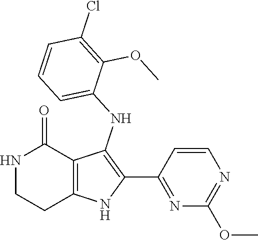 [SMILES: COc1nccc(-c2[nH]c3c(c2Nc2cccc(Cl)c2OC)C(=O)NCC3)n1] |
| 429a | 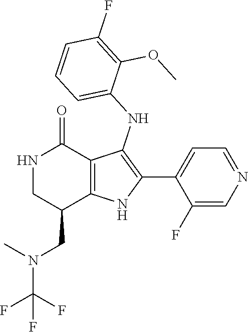 [SMILES: COc1c(F)cccc1Nc1c(-c2ccncc2F)[nH]c2c1C(=O)NC[C@@H]2CN(C)C(F)(F)F] |
| 429b | 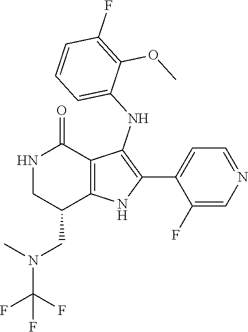 [SMILES: COc1c(F)cccc1Nc1c(-c2ccncc2F)[nH]c2c1C(=O)NC[C@H]2CN(C)C(F)(F)F] |
| 430 | 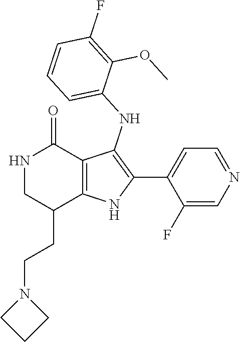 [SMILES: COc1c(F)cccc1Nc1c(-c2ccncc2F)[nH]c2c1C(=O)NCC2CCN1CCC1] |
| 287 | 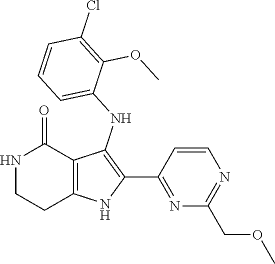 [SMILES: COCc1nccc(-c2[nH]c3c(c2Nc2cccc(Cl)c2OC)C(=O)NCC3)n1] |
| 288 | 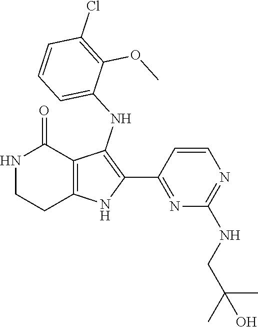 [SMILES: COc1c(Cl)cccc1Nc1c(-c2ccnc(NCC(C)(C)O)n2)[nH]c2c1C(=O)NCC2] |
| 289 | 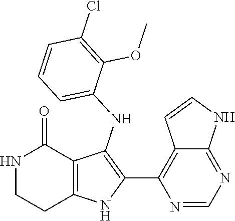 [SMILES: COc1c(Cl)cccc1Nc1c(-c2ncnc3[nH]ccc23)[nH]c2c1C(=O)NCC2] |
| 430a | 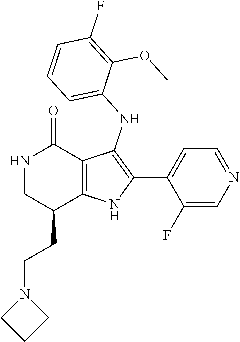 [SMILES: COc1c(F)cccc1Nc1c(-c2ccncc2F)[nH]c2c1C(=O)NC[C@@H]2CCN1CCC1] |
| 430b | 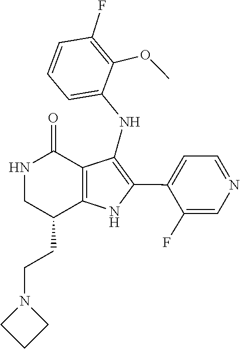 [SMILES: COc1c(F)cccc1Nc1c(-c2ccncc2F)[nH]c2c1C(=O)NC[C@H]2CCN1CCC1] |
| 431 | 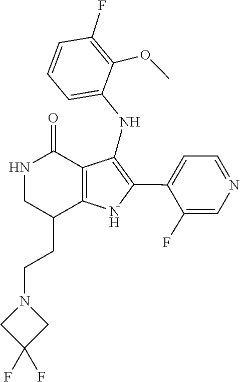 [SMILES: COc1c(F)cccc1Nc1c(-c2ccncc2F)[nH]c2c1C(=O)NCC2CCN1CC(F)(F)C1] |
| 290 | 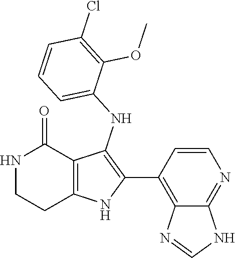 [SMILES: COc1c(Cl)cccc1Nc1c(-c2ccnc3[nH]cnc23)[nH]c2c1C(=O)NCC2] |
| 291 | 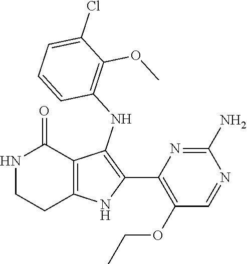 [SMILES: CCOc1cnc(N)nc1-c1[nH]c2c(c1Nc1cccc(Cl)c1OC)C(=O)NCC2] |
| 292 | 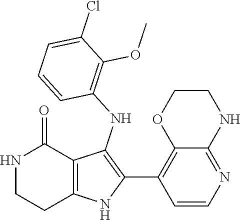 [SMILES: COc1c(Cl)cccc1Nc1c(-c2ccnc3c2OCCN3)[nH]c2c1C(=O)NCC2] |
| 431a | 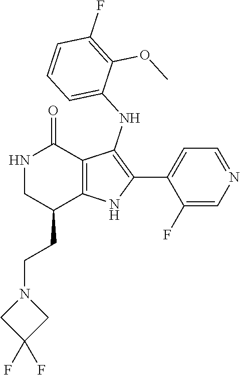 [SMILES: COc1c(F)cccc1Nc1c(-c2ccncc2F)[nH]c2c1C(=O)NC[C@@H]2CCN1CC(F)(F)C1] |
| 431b | 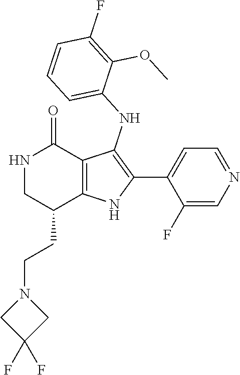 [SMILES: COc1c(F)cccc1Nc1c(-c2ccncc2F)[nH]c2c1C(=O)NC[C@H]2CCN1CC(F)(F)C1] |
| 432 | 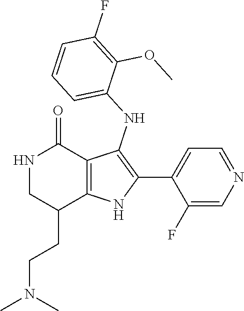 [SMILES: COc1c(F)cccc1Nc1c(-c2ccncc2F)[nH]c2c1C(=O)NCC2CCN(C)C] |
| 293 | 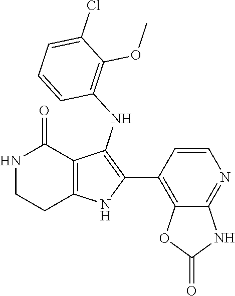 [SMILES: COc1c(Cl)cccc1Nc1c(-c2ccnc3[nH]c(=O)oc23)[nH]c2c1C(=O)NCC2] |
| 294 | 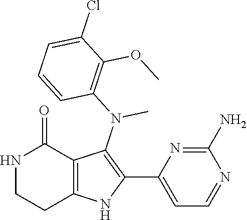 [SMILES: COc1c(Cl)cccc1N(C)c1c(-c2ccnc(N)n2)[nH]c2c1C(=O)NCC2] |
| 295 | 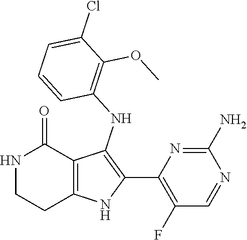 [SMILES: COc1c(Cl)cccc1Nc1c(-c2nc(N)ncc2F)[nH]c2c1C(=O)NCC2] |
| 296 | 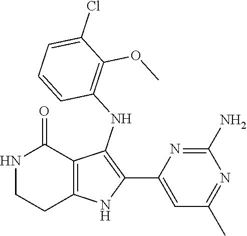 [SMILES: COc1c(Cl)cccc1Nc1c(-c2cc(C)nc(N)n2)[nH]c2c1C(=O)NCC2] |
| 432a | 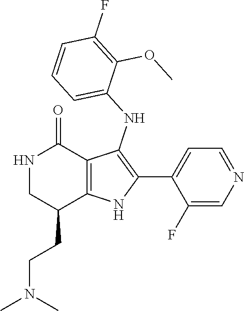 [SMILES: COc1c(F)cccc1Nc1c(-c2ccncc2F)[nH]c2c1C(=O)NC[C@@H]2CCN(C)C] |
| 432b | 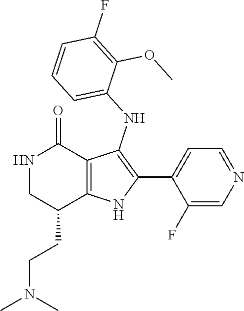 [SMILES: COc1c(F)cccc1Nc1c(-c2ccncc2F)[nH]c2c1C(=O)NC[C@H]2CCN(C)C] |
| 433 | 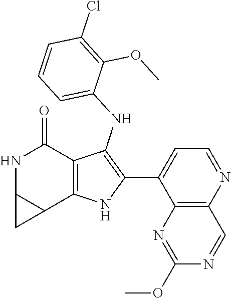 [SMILES: COc1ncc2nccc(-c3[nH]c4c(c3Nc3cccc(Cl)c3OC)C(=O)NC3CC43)c2n1] |
| 433a | 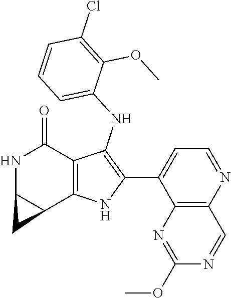 [SMILES: COc1ncc2nccc(-c3[nH]c4c(c3Nc3cccc(Cl)c3OC)C(=O)N[C@@H]3C[C@H]43)c2n1] |
| 297 | 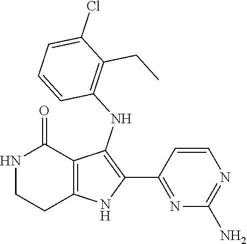 [SMILES: CCc1c(Cl)cccc1Nc1c(-c2ccnc(N)n2)[nH]c2c1C(=O)NCC2] |
| 298 | 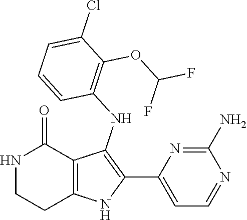 [SMILES: Nc1nccc(-c2[nH]c3c(c2Nc2cccc(Cl)c2OC(F)F)C(=O)NCC3)n1] |
| 299 | 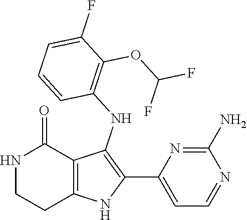 [SMILES: Nc1nccc(-c2[nH]c3c(c2Nc2cccc(F)c2OC(F)F)C(=O)NCC3)n1] |
| 300 | 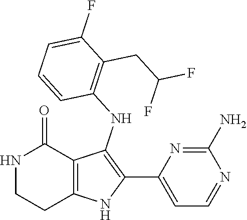 [SMILES: Nc1nccc(-c2[nH]c3c(c2Nc2cccc(F)c2CC(F)F)C(=O)NCC3)n1] |
| 433b | 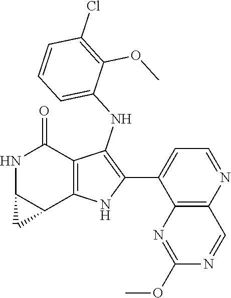 [SMILES: COc1ncc2nccc(-c3[nH]c4c(c3Nc3cccc(Cl)c3OC)C(=O)N[C@H]3C[C@@H]43)c2n1] |
| 434 | 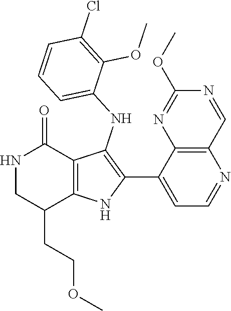 [SMILES: COCCC1CNC(=O)c2c1[nH]c(-c1ccnc3cnc(OC)nc13)c2Nc1cccc(Cl)c1OC] |
| 434a | 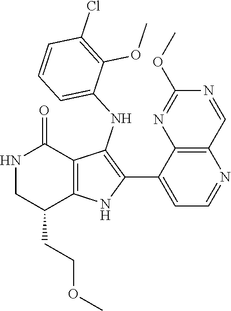 [SMILES: COCC[C@@H]1CNC(=O)c2c1[nH]c(-c1ccnc3cnc(OC)nc13)c2Nc1cccc(Cl)c1OC] |
| 434b | 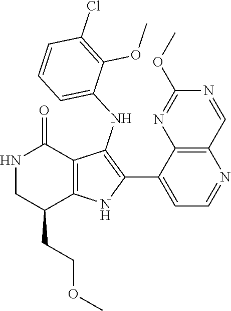 [SMILES: COCC[C@H]1CNC(=O)c2c1[nH]c(-c1ccnc3cnc(OC)nc13)c2Nc1cccc(Cl)c1OC] |
| 301 | 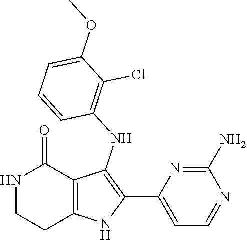 [SMILES: COc1cccc(Nc2c(-c3ccnc(N)n3)[nH]c3c2C(=O)NCC3)c1Cl] |
| 302 | 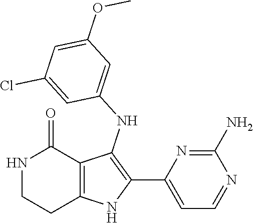 [SMILES: COc1cc(Cl)cc(Nc2c(-c3ccnc(N)n3)[nH]c3c2C(=O)NCC3)c1] |
| 303 | 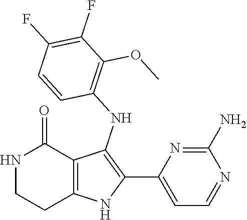 [SMILES: COc1c(Nc2c(-c3ccnc(N)n3)[nH]c3c2C(=O)NCC3)ccc(F)c1F] |
| 304 | 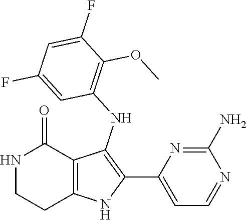 [SMILES: COc1c(F)cc(F)cc1Nc1c(-c2ccnc(N)n2)[nH]c2c1C(=O)NCC2] |
| 435 | 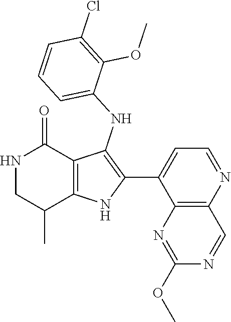 [SMILES: COc1ncc2nccc(-c3[nH]c4c(c3Nc3cccc(Cl)c3OC)C(=O)NCC4C)c2n1] |
| 435a | 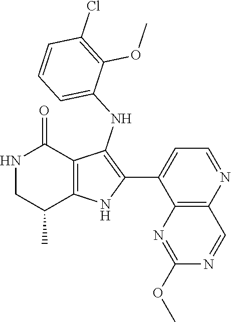 [SMILES: COc1ncc2nccc(-c3[nH]c4c(c3Nc3cccc(Cl)c3OC)C(=O)NC[C@H]4C)c2n1] |
| 435b | 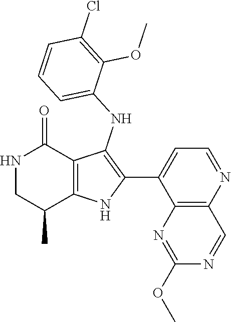 [SMILES: COc1ncc2nccc(-c3[nH]c4c(c3Nc3cccc(Cl)c3OC)C(=O)NC[C@@H]4C)c2n1] |
| 436 | 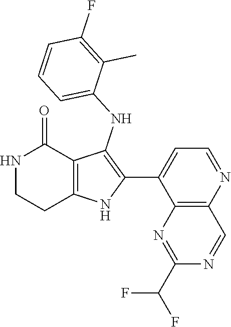 [SMILES: Cc1c(F)cccc1Nc1c(-c2ccnc3cnc(C(F)F)nc23)[nH]c2c1C(=O)NCC2] |
| 305 | 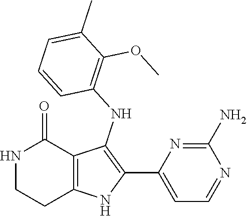 [SMILES: COc1c(C)cccc1Nc1c(-c2ccnc(N)n2)[nH]c2c1C(=O)NCC2] |
| 306 | 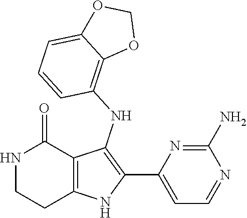 [SMILES: Nc1nccc(-c2[nH]c3c(c2Nc2cccc4c2OCO4)C(=O)NCC3)n1] |
| 307 | 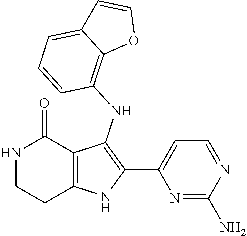 [SMILES: Nc1nccc(-c2[nH]c3c(c2Nc2cccc4ccoc24)C(=O)NCC3)n1] |
| 437 | 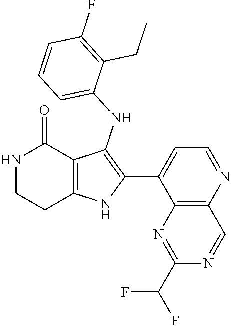 [SMILES: CCc1c(F)cccc1Nc1c(-c2ccnc3cnc(C(F)F)nc23)[nH]c2c1C(=O)NCC2] |
| 438 | 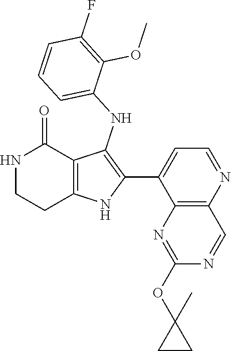 [SMILES: COc1c(F)cccc1Nc1c(-c2ccnc3cnc(OC4(C)CC4)nc23)[nH]c2c1C(=O)NCC2] |
| 439 | 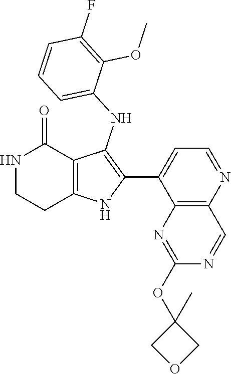 [SMILES: COc1c(F)cccc1Nc1c(-c2ccnc3cnc(OC4(C)COC4)nc23)[nH]c2c1C(=O)NCC2] |
| 308 | 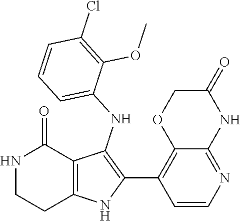 [SMILES: COc1c(Cl)cccc1Nc1c(-c2ccnc3c2OCC(=O)N3)[nH]c2c1C(=O)NCC2] |
| 309 | 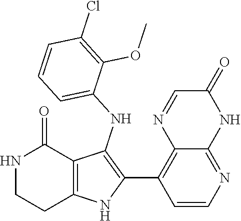 [SMILES: COc1c(Cl)cccc1Nc1c(-c2ccnc3[nH]c(=O)cnc23)[nH]c2c1C(=O)NCC2] |
| 310 | 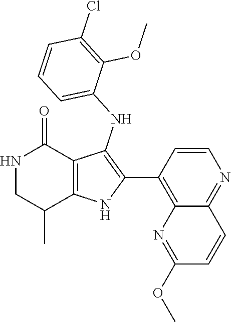 [SMILES: COc1ccc2nccc(-c3[nH]c4c(c3Nc3cccc(Cl)c3OC)C(=O)NCC4C)c2n1] |
| 440 | 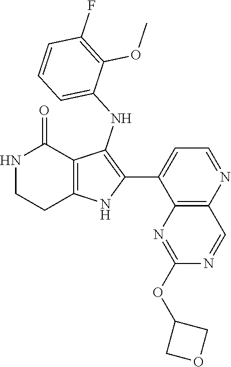 [SMILES: COc1c(F)cccc1Nc1c(-c2ccnc3cnc(OC4COC4)nc23)[nH]c2c1C(=O)NCC2] |
| 441 | 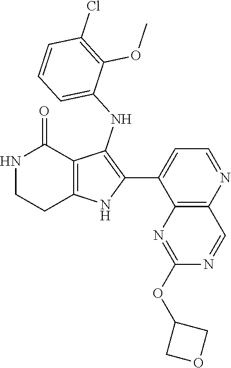 [SMILES: COc1c(Cl)cccc1Nc1c(-c2ccnc3cnc(OC4COC4)nc23)[nH]c2c1C(=O)NCC2] |
| 442 | 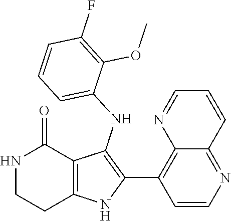 [SMILES: COc1c(F)cccc1Nc1c(-c2ccnc3cccnc23)[nH]c2c1C(=O)NCC2] |
| 310a | 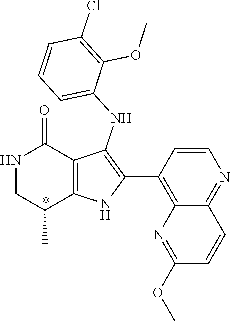 [SMILES: COc1ccc2nccc(-c3[nH]c4c(c3Nc3cccc(Cl)c3OC)C(=O)NC[C@H]4C)c2n1] |
| 310b | 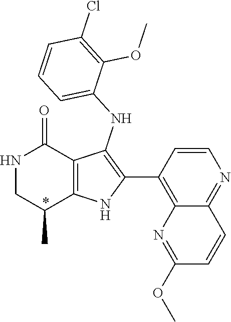 [SMILES: COc1ccc2nccc(-c3[nH]c4c(c3Nc3cccc(Cl)c3OC)C(=O)NC[C@@H]4C)c2n1] |
| 311 | 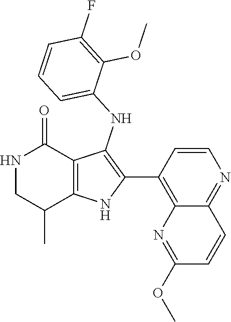 [SMILES: COc1ccc2nccc(-c3[nH]c4c(c3Nc3cccc(F)c3OC)C(=O)NCC4C)c2n1] |
| 311a | 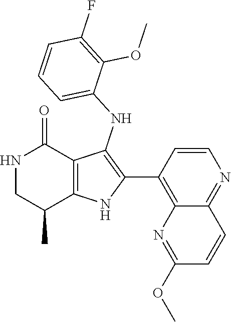 [SMILES: COc1ccc2nccc(-c3[nH]c4c(c3Nc3cccc(F)c3OC)C(=O)NC[C@@H]4C)c2n1] |
| 443 | 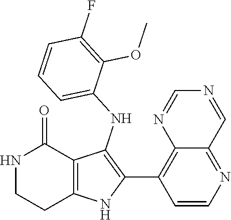 [SMILES: COc1c(F)cccc1Nc1c(-c2ccnc3cncnc23)[nH]c2c1C(=O)NCC2] |
| 444 | 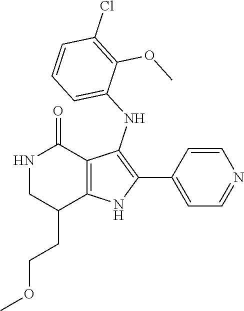 [SMILES: COCCC1CNC(=O)c2c1[nH]c(-c1ccncc1)c2Nc1cccc(Cl)c1OC] |
| 444a | 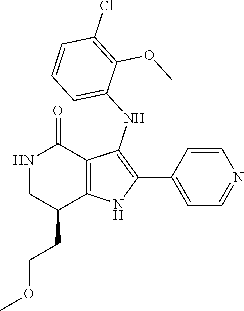 [SMILES: COCC[C@H]1CNC(=O)c2c1[nH]c(-c1ccncc1)c2Nc1cccc(Cl)c1OC] |
| 444b | 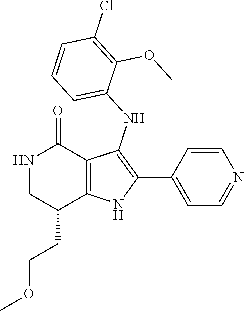 [SMILES: COCC[C@@H]1CNC(=O)c2c1[nH]c(-c1ccncc1)c2Nc1cccc(Cl)c1OC] |
| 311b | 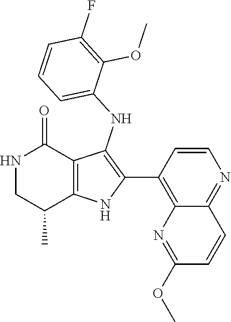 [SMILES: COc1ccc2nccc(-c3[nH]c4c(c3Nc3cccc(F)c3OC)C(=O)NC[C@H]4C)c2n1] |
| 312 | 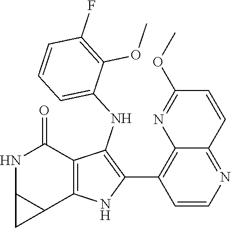 [SMILES: COc1ccc2nccc(-c3[nH]c4c(c3Nc3cccc(F)c3OC)C(=O)NC3CC43)c2n1] |
| 312a | 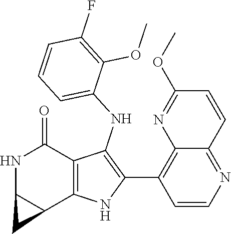 [SMILES: COc1ccc2nccc(-c3[nH]c4c(c3Nc3cccc(F)c3OC)C(=O)N[C@@H]3C[C@H]43)c2n1] |
| 312b | 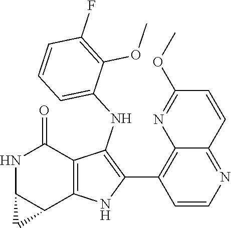 [SMILES: COc1ccc2nccc(-c3[nH]c4c(c3Nc3cccc(F)c3OC)C(=O)N[C@H]3C[C@@H]43)c2n1] |
| 445 | 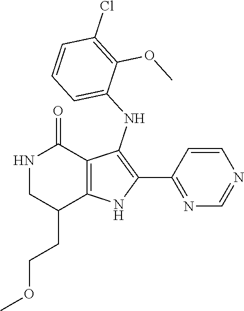 [SMILES: COCCC1CNC(=O)c2c1[nH]c(-c1ccncn1)c2Nc1cccc(Cl)c1OC] |
| 445a | 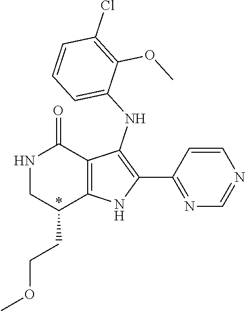 [SMILES: COCC[C@@H]1CNC(=O)c2c1[nH]c(-c1ccncn1)c2Nc1cccc(Cl)c1OC] |
| 445b | 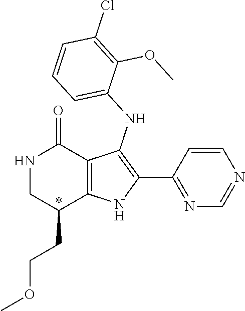 [SMILES: COCC[C@H]1CNC(=O)c2c1[nH]c(-c1ccncn1)c2Nc1cccc(Cl)c1OC] |
| 446 | 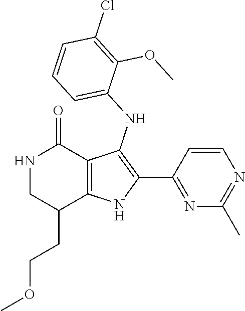 |
| 313 | 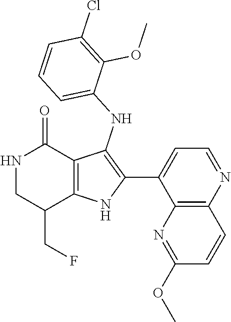 [SMILES: COc1ccc2nccc(-c3[nH]c4c(c3Nc3cccc(Cl)c3OC)C(=O)NCC4CF)c2n1] |
| 313a | 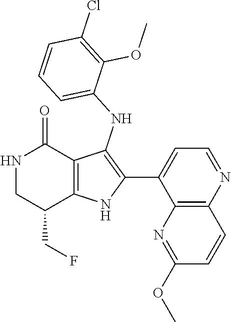 [SMILES: COc1ccc2nccc(-c3[nH]c4c(c3Nc3cccc(Cl)c3OC)C(=O)NC[C@H]4CF)c2n1] |
| 313b | 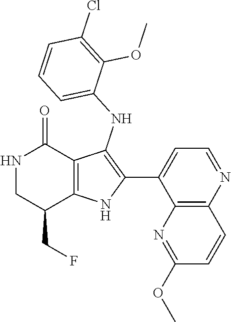 [SMILES: COc1ccc2nccc(-c3[nH]c4c(c3Nc3cccc(Cl)c3OC)C(=O)NC[C@@H]4CF)c2n1] |
| 314 | 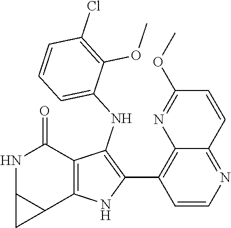 [SMILES: COc1ccc2nccc(-c3[nH]c4c(c3Nc3cccc(Cl)c3OC)C(=O)NC3CC43)c2n1] |
| 446a | 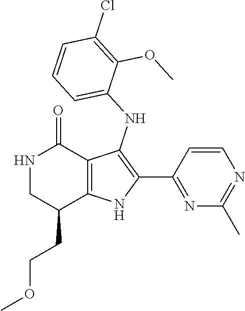 |
| 446b | 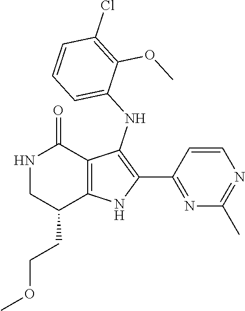 |
| 447 | 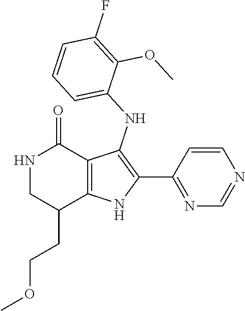 [SMILES: COCCC1CNC(=O)c2c1[nH]c(-c1ccncn1)c2Nc1cccc(F)c1OC] |
| 447a | 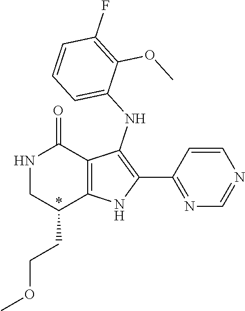 [SMILES: COCC[C@@H]1CNC(=O)c2c1[nH]c(-c1ccncn1)c2Nc1cccc(F)c1OC] |
| 314a | 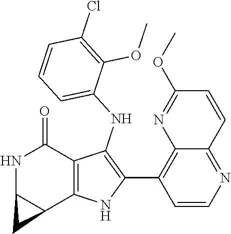 [SMILES: COc1ccc2nccc(-c3[nH]c4c(c3Nc3cccc(Cl)c3OC)C(=O)N[C@@H]3C[C@H]43)c2n1] |
| 314b | 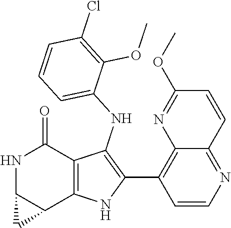 [SMILES: COc1ccc2nccc(-c3[nH]c4c(c3Nc3cccc(Cl)c3OC)C(=O)N[C@H]3C[C@@H]43)c2n1] |
| 315 | 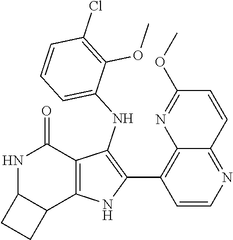 [SMILES: COc1ccc2nccc(-c3[nH]c4c(c3Nc3cccc(Cl)c3OC)C(=O)NC3CCC43)c2n1] |
| 315a | 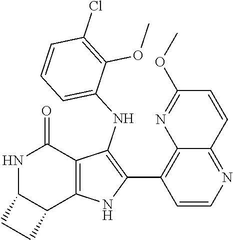 [SMILES: COc1ccc2nccc(-c3[nH]c4c(c3Nc3cccc(Cl)c3OC)C(=O)N[C@H]3CC[C@@H]43)c2n1] |
| 447b | 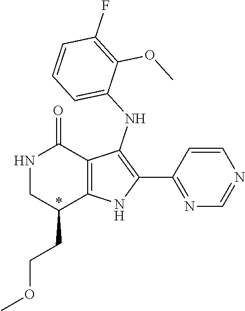 [SMILES: COCC[C@H]1CNC(=O)c2c1[nH]c(-c1ccncn1)c2Nc1cccc(F)c1OC] |
| 448 | 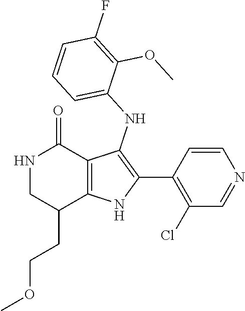 [SMILES: COCCC1CNC(=O)c2c1[nH]c(-c1ccncc1Cl)c2Nc1cccc(F)c1OC] |
| 448a | 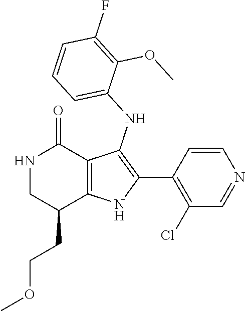 [SMILES: COCC[C@H]1CNC(=O)c2c1[nH]c(-c1ccncc1Cl)c2Nc1cccc(F)c1OC] |
| 448b | 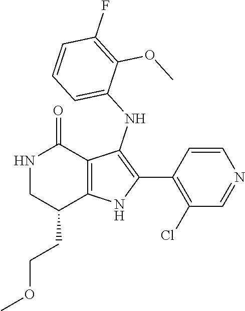 [SMILES: COCC[C@@H]1CNC(=O)c2c1[nH]c(-c1ccncc1Cl)c2Nc1cccc(F)c1OC] |
| 315b | 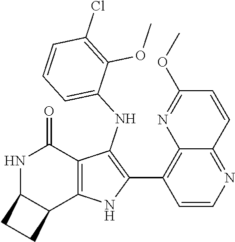 [SMILES: COc1ccc2nccc(-c3[nH]c4c(c3Nc3cccc(Cl)c3OC)C(=O)N[C@@H]3CC[C@H]43)c2n1] |
| 316 | 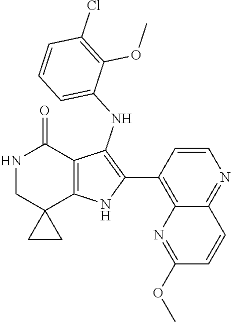 [SMILES: COc1ccc2nccc(-c3[nH]c4c(c3Nc3cccc(Cl)c3OC)C(=O)NCC43CC3)c2n1] |
| 317 | 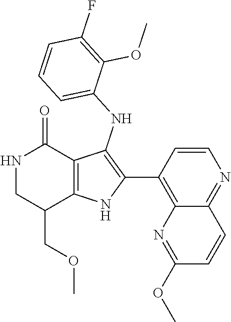 [SMILES: COCC1CNC(=O)c2c1[nH]c(-c1ccnc3ccc(OC)nc13)c2Nc1cccc(F)c1OC] |
| 317a | 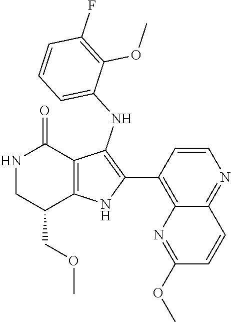 [SMILES: COC[C@@H]1CNC(=O)c2c1[nH]c(-c1ccnc3ccc(OC)nc13)c2Nc1cccc(F)c1OC] |
| 449 | 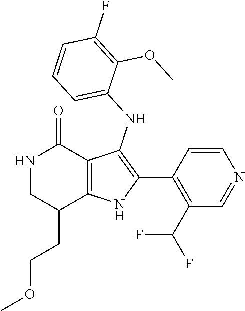 [SMILES: COCCC1CNC(=O)c2c1[nH]c(-c1ccncc1C(F)F)c2Nc1cccc(F)c1OC] |
| 449a | 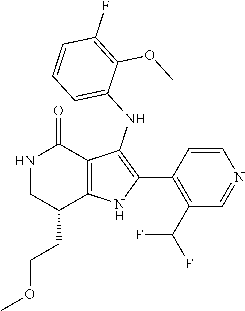 [SMILES: COCC[C@@H]1CNC(=O)c2c1[nH]c(-c1ccncc1C(F)F)c2Nc1cccc(F)c1OC] |
| 449b | 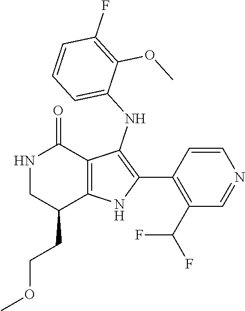 [SMILES: COCC[C@H]1CNC(=O)c2c1[nH]c(-c1ccncc1C(F)F)c2Nc1cccc(F)c1OC] |
| 450 | 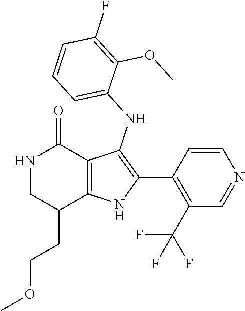 [SMILES: COCCC1CNC(=O)c2c1[nH]c(-c1ccncc1C(F)(F)F)c2Nc1cccc(F)c1OC] |
| 317b | 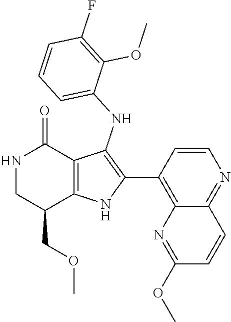 [SMILES: COC[C@H]1CNC(=O)c2c1[nH]c(-c1ccnc3ccc(OC)nc13)c2Nc1cccc(F)c1OC] |
| 318 | 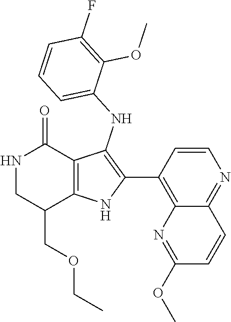 [SMILES: CCOCC1CNC(=O)c2c1[nH]c(-c1ccnc3ccc(OC)nc13)c2Nc1cccc(F)c1OC] |
| 318a | 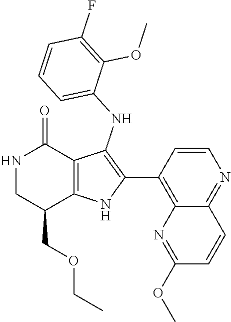 [SMILES: CCOC[C@H]1CNC(=O)c2c1[nH]c(-c1ccnc3ccc(OC)nc13)c2Nc1cccc(F)c1OC] |
| 318b | 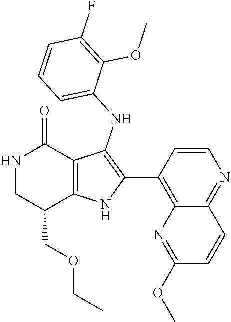 [SMILES: CCOC[C@@H]1CNC(=O)c2c1[nH]c(-c1ccnc3ccc(OC)nc13)c2Nc1cccc(F)c1OC] |
| 450a | 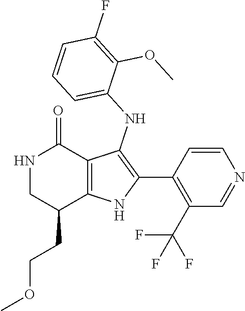 [SMILES: COCC[C@H]1CNC(=O)c2c1[nH]c(-c1ccncc1C(F)(F)F)c2Nc1cccc(F)c1OC] |
| 450b | 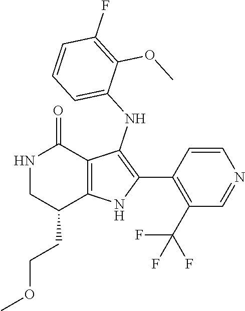 [SMILES: COCC[C@@H]1CNC(=O)c2c1[nH]c(-c1ccncc1C(F)(F)F)c2Nc1cccc(F)c1OC] |
| 451 | 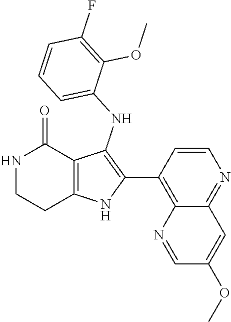 [SMILES: COc1cnc2c(-c3[nH]c4c(c3Nc3cccc(F)c3OC)C(=O)NCC4)ccnc2c1] |
| 452 | 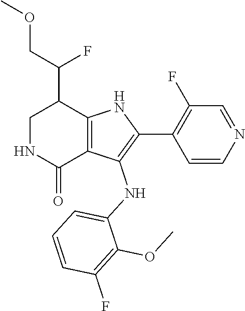 [SMILES: COCC(F)C1CNC(=O)c2c1[nH]c(-c1ccncc1F)c2Nc1cccc(F)c1OC] |
| 452a | 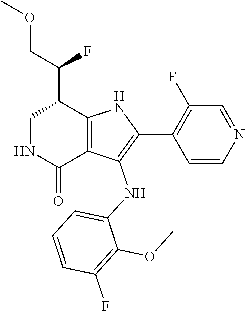 [SMILES: COC[C@@H](F)[C@H]1CNC(=O)c2c1[nH]c(-c1ccncc1F)c2Nc1cccc(F)c1OC] |
| 452b | 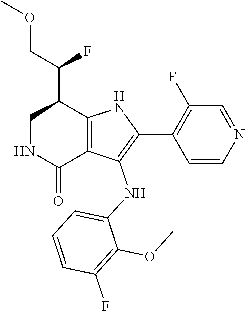 [SMILES: COC[C@@H](F)[C@@H]1CNC(=O)c2c1[nH]c(-c1ccncc1F)c2Nc1cccc(F)c1OC] |
| 452c | 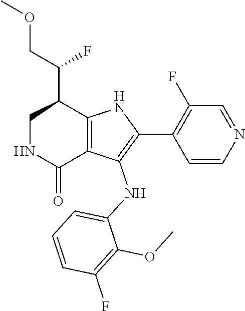 [SMILES: COC[C@H](F)[C@@H]1CNC(=O)c2c1[nH]c(-c1ccncc1F)c2Nc1cccc(F)c1OC] |
| 461 | 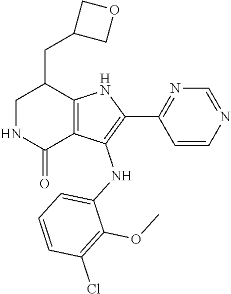 [SMILES: COc1c(Cl)cccc1Nc1c(-c2ccncn2)[nH]c2c1C(=O)NCC2CC1COC1] |
| 461a | 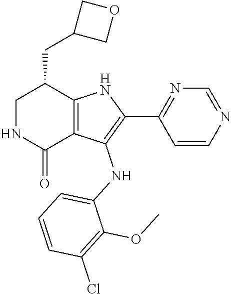 [SMILES: COc1c(Cl)cccc1Nc1c(-c2ccncn2)[nH]c2c1C(=O)NC[C@@H]2CC1COC1] |
| 461b | 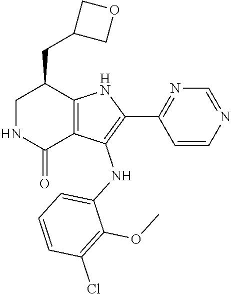 [SMILES: COc1c(Cl)cccc1Nc1c(-c2ccncn2)[nH]c2c1C(=O)NC[C@H]2CC1COC1] |
| 462 | 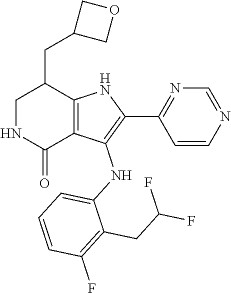 [SMILES: O=C1NCC(CC2COC2)c2[nH]c(-c3ccncn3)c(Nc3cccc(F)c3CC(F)F)c21] |
| 452d | 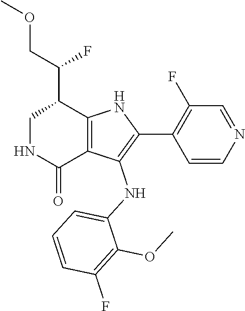 [SMILES: COC[C@H](F)[C@H]1CNC(=O)c2c1[nH]c(-c1ccncc1F)c2Nc1cccc(F)c1OC] |
| 453 | 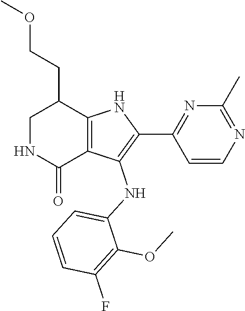 |
| 453a | 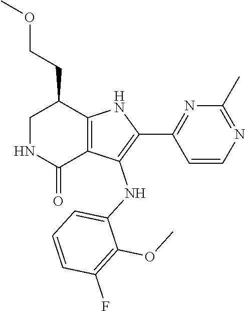 |
| 453b | 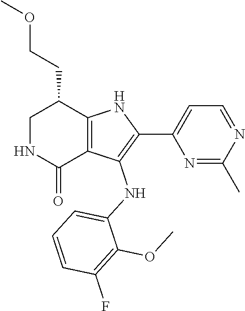 |
| 462a | 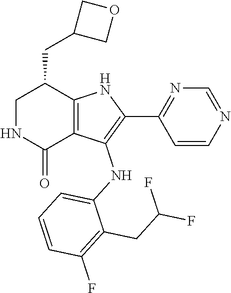 [SMILES: O=C1NC[C@H](CC2COC2)c2[nH]c(-c3ccncn3)c(Nc3cccc(F)c3CC(F)F)c21] |
| 462b | 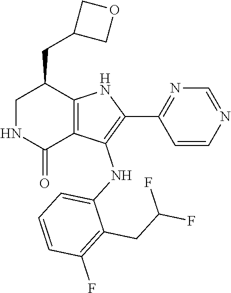 [SMILES: O=C1NC[C@@H](CC2COC2)c2[nH]c(-c3ccncn3)c(Nc3cccc(F)c3CC(F)F)c21] |
| 463 | 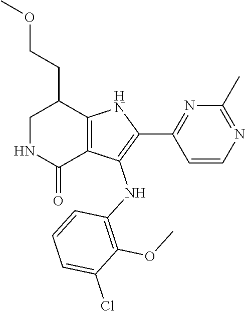 |
| 463a | 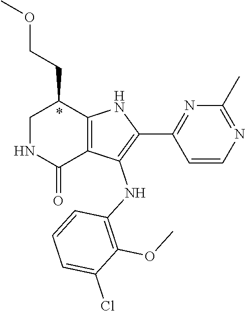 |
| 454 | 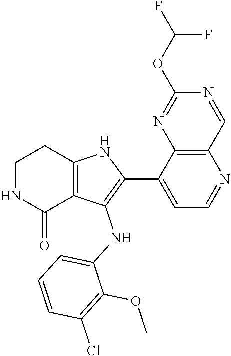 [SMILES: COc1c(Cl)cccc1Nc1c(-c2ccnc3cnc(OC(F)F)nc23)[nH]c2c1C(=O)NCC2] |
| 455 | 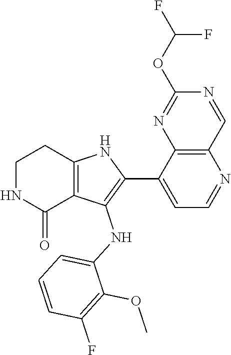 [SMILES: COc1c(F)cccc1Nc1c(-c2ccnc3cnc(OC(F)F)nc23)[nH]c2c1C(=O)NCC2] |
| 456 | 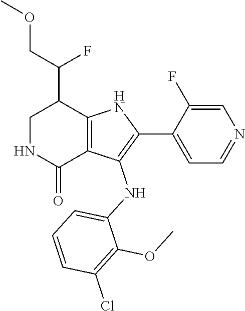 [SMILES: COCC(F)C1CNC(=O)c2c1[nH]c(-c1ccncc1F)c2Nc1cccc(Cl)c1OC] |
| 463b | 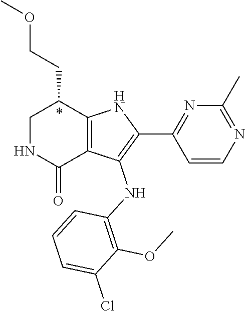 |
| 464 | 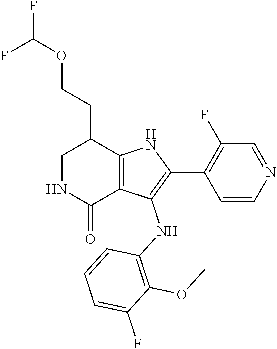 [SMILES: COc1c(F)cccc1Nc1c(-c2ccncc2F)[nH]c2c1C(=O)NCC2CCOC(F)F] |
| 464a | 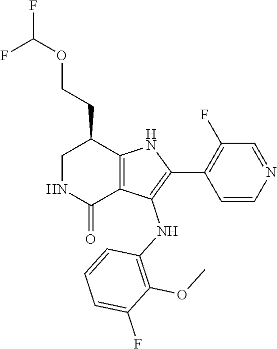 [SMILES: COc1c(F)cccc1Nc1c(-c2ccncc2F)[nH]c2c1C(=O)NC[C@H]2CCOC(F)F] |
| 456a | 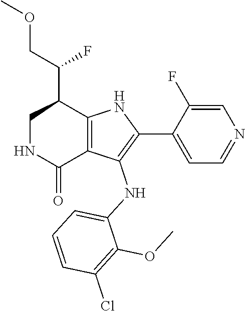 [SMILES: COC[C@H](F)[C@@H]1CNC(=O)c2c1[nH]c(-c1ccncc1F)c2Nc1cccc(Cl)c1OC] |
| 456b | 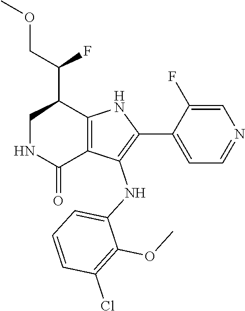 [SMILES: COC[C@@H](F)[C@@H]1CNC(=O)c2c1[nH]c(-c1ccncc1F)c2Nc1cccc(Cl)c1OC] |
| 456c | 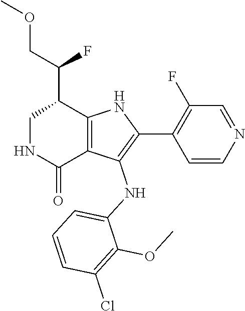 [SMILES: COC[C@@H](F)[C@H]1CNC(=O)c2c1[nH]c(-c1ccncc1F)c2Nc1cccc(Cl)c1OC] |
| 464b | 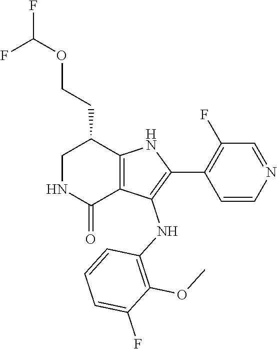 [SMILES: COc1c(F)cccc1Nc1c(-c2ccncc2F)[nH]c2c1C(=O)NC[C@@H]2CCOC(F)F] |
| 465 | 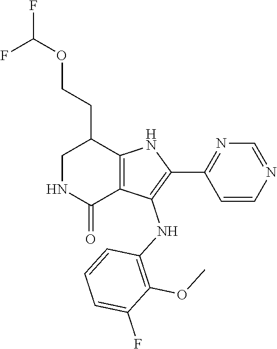 [SMILES: COc1c(F)cccc1Nc1c(-c2ccncn2)[nH]c2c1C(=O)NCC2CCOC(F)F] |
| 465a | 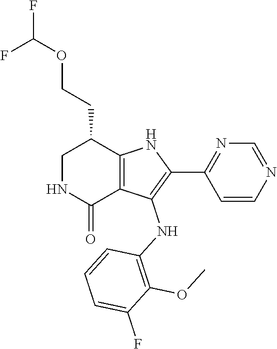 [SMILES: COc1c(F)cccc1Nc1c(-c2ccncn2)[nH]c2c1C(=O)NC[C@@H]2CCOC(F)F] |
| 456d | 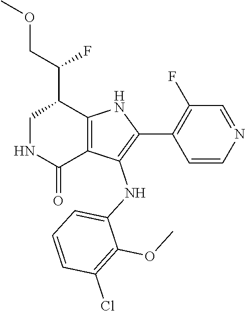 [SMILES: COC[C@H](F)[C@H]1CNC(=O)c2c1[nH]c(-c1ccncc1F)c2Nc1cccc(Cl)c1OC] |
| 457 | 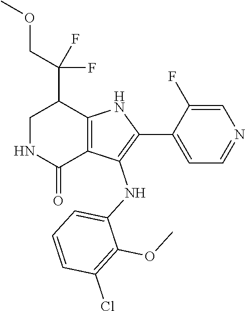 [SMILES: COCC(F)(F)C1CNC(=O)c2c1[nH]c(-c1ccncc1F)c2Nc1cccc(Cl)c1OC] |
| 457a | 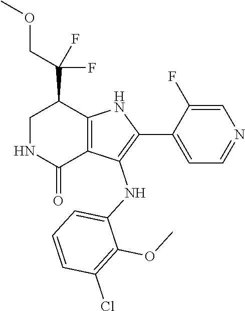 [SMILES: COCC(F)(F)[C@@H]1CNC(=O)c2c1[nH]c(-c1ccncc1F)c2Nc1cccc(Cl)c1OC] |
| 465b | 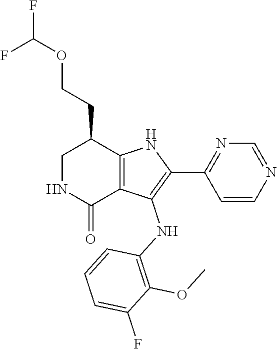 [SMILES: COc1c(F)cccc1Nc1c(-c2ccncn2)[nH]c2c1C(=O)NC[C@H]2CCOC(F)F] |
| 466 | 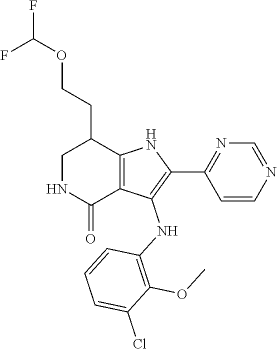 [SMILES: COc1c(Cl)cccc1Nc1c(-c2ccncn2)[nH]c2c1C(=O)NCC2CCOC(F)F] |
| 466a | 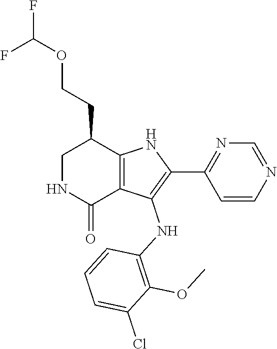 [SMILES: COc1c(Cl)cccc1Nc1c(-c2ccncn2)[nH]c2c1C(=O)NC[C@H]2CCOC(F)F] |
| 457b | 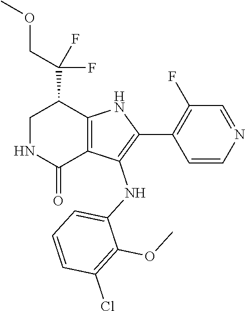 [SMILES: COCC(F)(F)[C@H]1CNC(=O)c2c1[nH]c(-c1ccncc1F)c2Nc1cccc(Cl)c1OC] |
| 458 | 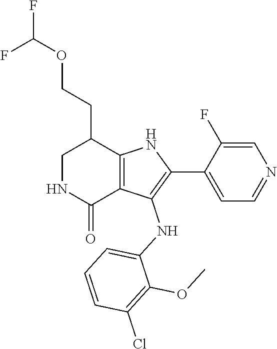 [SMILES: COc1c(Cl)cccc1Nc1c(-c2ccncc2F)[nH]c2c1C(=O)NCC2CCOC(F)F] |
| 458a | 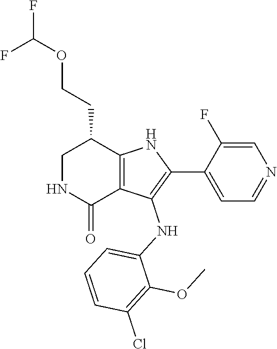 [SMILES: COc1c(Cl)cccc1Nc1c(-c2ccncc2F)[nH]c2c1C(=O)NC[C@@H]2CCOC(F)F] |
| 466b | 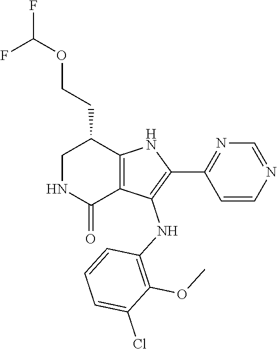 [SMILES: COc1c(Cl)cccc1Nc1c(-c2ccncn2)[nH]c2c1C(=O)NC[C@@H]2CCOC(F)F] |
| 467 | 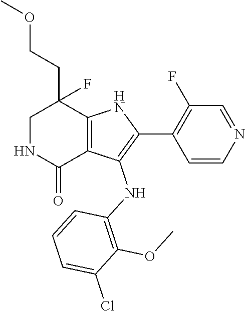 [SMILES: COCCC1(F)CNC(=O)c2c1[nH]c(-c1ccncc1F)c2Nc1cccc(Cl)c1OC] |
| 467a | 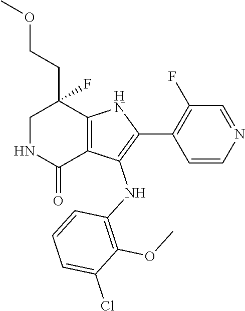 [SMILES: COCC[C@]1(F)CNC(=O)c2c1[nH]c(-c1ccncc1F)c2Nc1cccc(Cl)c1OC] |
| 458b | 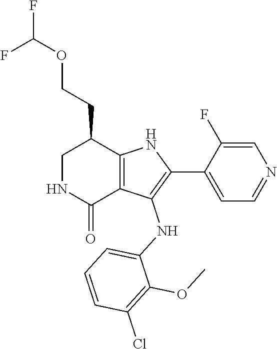 [SMILES: COc1c(Cl)cccc1Nc1c(-c2ccncc2F)[nH]c2c1C(=O)NC[C@H]2CCOC(F)F] |
| 459 | 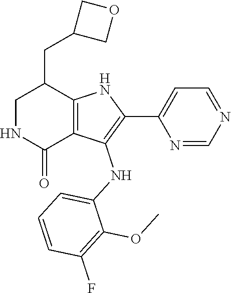 [SMILES: COc1c(F)cccc1Nc1c(-c2ccncn2)[nH]c2c1C(=O)NCC2CC1COC1] |
| 459a | 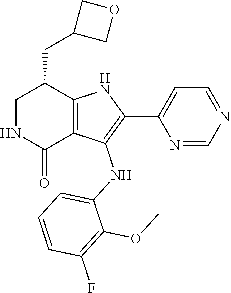 [SMILES: COc1c(F)cccc1Nc1c(-c2ccncn2)[nH]c2c1C(=O)NC[C@@H]2CC1COC1] |
| 459b | 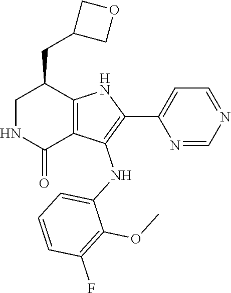 [SMILES: COc1c(F)cccc1Nc1c(-c2ccncn2)[nH]c2c1C(=O)NC[C@H]2CC1COC1] |
| 467b | 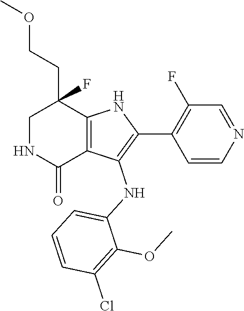 [SMILES: COCC[C@@]1(F)CNC(=O)c2c1[nH]c(-c1ccncc1F)c2Nc1cccc(Cl)c1OC] |
| 468 | 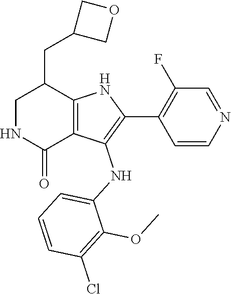 [SMILES: COc1c(Cl)cccc1Nc1c(-c2ccncc2F)[nH]c2c1C(=O)NCC2CC1COC1] |
| 468a | 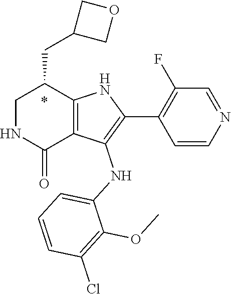 [SMILES: COc1c(Cl)cccc1Nc1c(-c2ccncc2F)[nH]c2c1C(=O)NC[C@@H]2CC1COC1] |
| 468b | 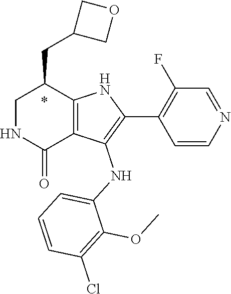 [SMILES: COc1c(Cl)cccc1Nc1c(-c2ccncc2F)[nH]c2c1C(=O)NC[C@H]2CC1COC1] |
| 460 | 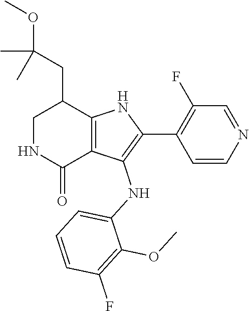 [SMILES: COc1c(F)cccc1Nc1c(-c2ccncc2F)[nH]c2c1C(=O)NCC2CC(C)(C)OC] |
| 460a | 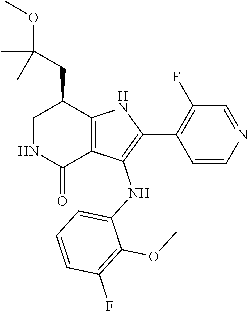 [SMILES: COc1c(F)cccc1Nc1c(-c2ccncc2F)[nH]c2c1C(=O)NC[C@H]2CC(C)(C)OC] |
| 460b | 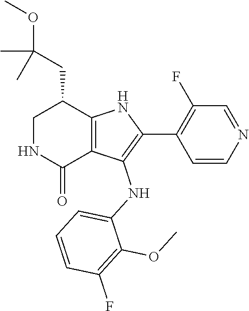 [SMILES: COc1c(F)cccc1Nc1c(-c2ccncc2F)[nH]c2c1C(=O)NC[C@@H]2CC(C)(C)OC] |
| 470 | 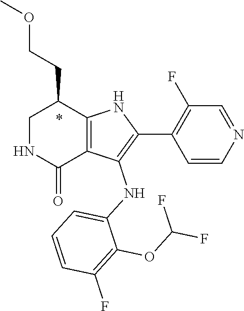 [SMILES: COCC[C@@H]1CNC(=O)c2c1[nH]c(-c1ccncc1F)c2Nc1cccc(F)c1OC(F)F] |
| 469 | 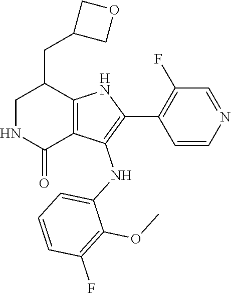 [SMILES: COc1c(F)cccc1Nc1c(-c2ccncc2F)[nH]c2c1C(=O)NCC2CC1COC1] |
| 469a | 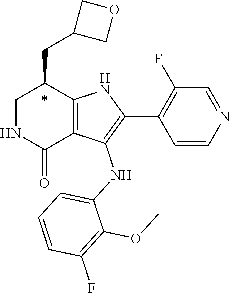 [SMILES: COc1c(F)cccc1Nc1c(-c2ccncc2F)[nH]c2c1C(=O)NC[C@H]2CC1COC1] |
| 469b | 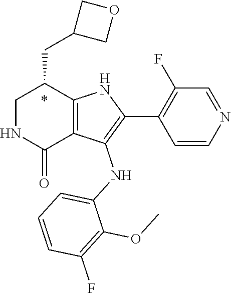 [SMILES: COc1c(F)cccc1Nc1c(-c2ccncc2F)[nH]c2c1C(=O)NC[C@@H]2CC1COC1] |
| 651 | 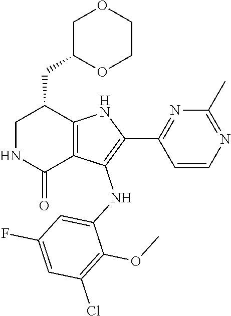 [SMILES: COc1c(Cl)cc(F)cc1Nc1c(-c2ccnc(C)n2)[nH]c2c1C(=O)NC[C@@H]2C[C@@H]1COCCO1] |
| 471 | 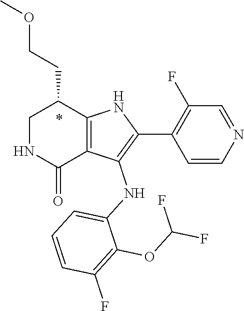 [SMILES: COCC[C@H]1CNC(=O)c2c1[nH]c(-c1ccncc1F)c2Nc1cccc(F)c1OC(F)F] |
| 472 | 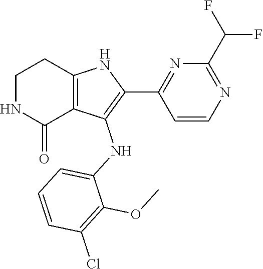 [SMILES: COc1c(Cl)cccc1Nc1c(-c2ccnc(C(F)F)n2)[nH]c2c1C(=O)NCC2] |
| 473 | 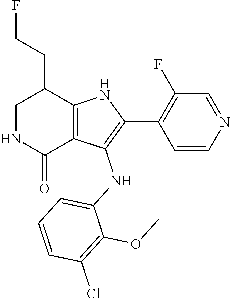 [SMILES: COc1c(Cl)cccc1Nc1c(-c2ccncc2F)[nH]c2c1C(=O)NCC2CCF] |
| 474 | 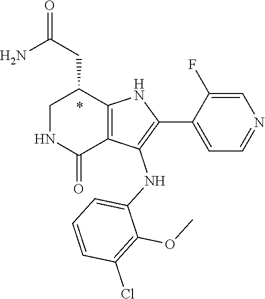 [SMILES: COc1c(Cl)cccc1Nc1c(-c2ccncc2F)[nH]c2c1C(=O)NC[C@@H]2CC(N)=O] |
| 652 | 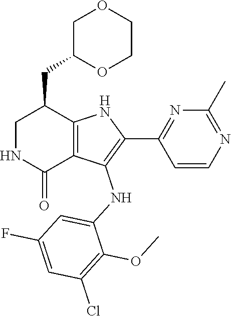 [SMILES: COc1c(Cl)cc(F)cc1Nc1c(-c2ccnc(C)n2)[nH]c2c1C(=O)NC[C@H]2C[C@@H]1COCCO1] |
| 653 | 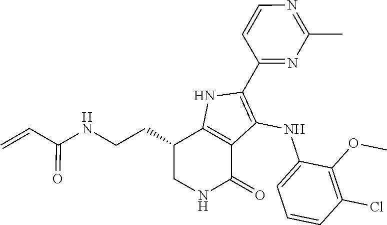 [SMILES: C=CC(=O)NCC[C@H]1CNC(=O)c2c1[nH]c(-c1ccnc(C)n1)c2Nc1cccc(Cl)c1OC] |
| 654 | 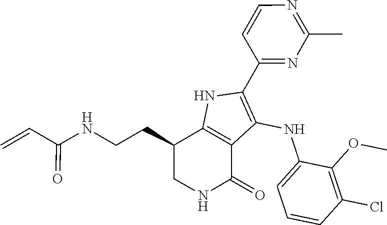 [SMILES: C=CC(=O)NCC[C@@H]1CNC(=O)c2c1[nH]c(-c1ccnc(C)n1)c2Nc1cccc(Cl)c1OC] |
| 655 | 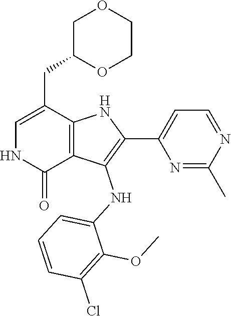 [SMILES: COc1c(Cl)cccc1Nc1c(-c2ccnc(C)n2)[nH]c2c(C[C@@H]3COCCO3)c[nH]c(=O)c12] |
| 475 | 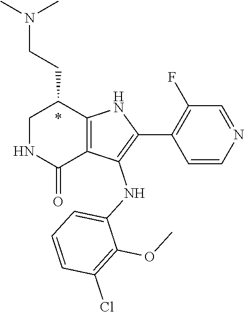 [SMILES: COc1c(Cl)cccc1Nc1c(-c2ccncc2F)[nH]c2c1C(=O)NC[C@@H]2CCN(C)C] |
| 476 | 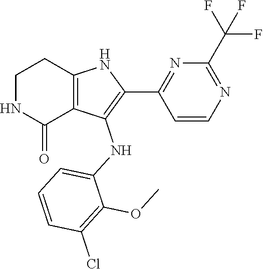 [SMILES: COc1c(Cl)cccc1Nc1c(-c2ccnc(C(F)(F)F)n2)[nH]c2c1C(=O)NCC2] |
| 477 | 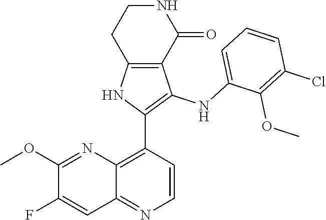 [SMILES: COc1nc2c(-c3[nH]c4c(c3Nc3cccc(Cl)c3OC)C(=O)NCC4)ccnc2cc1F] |
| 656 | 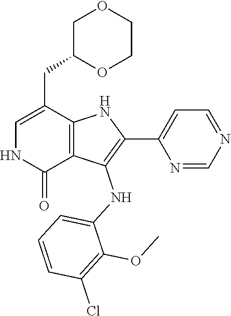 [SMILES: COc1c(Cl)cccc1Nc1c(-c2ccncn2)[nH]c2c(C[C@@H]3COCCO3)c[nH]c(=O)c12] |
| 657 | 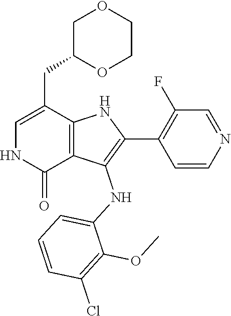 [SMILES: COc1c(Cl)cccc1Nc1c(-c2ccncc2F)[nH]c2c(C[C@@H]3COCCO3)c[nH]c(=O)c12] |
| 658 | 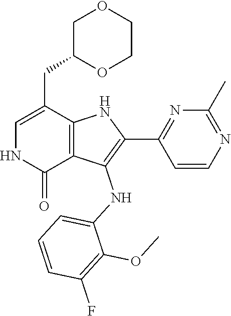 [SMILES: COc1c(F)cccc1Nc1c(-c2ccnc(C)n2)[nH]c2c(C[C@@H]3COCCO3)c[nH]c(=O)c12] |
| 478 | 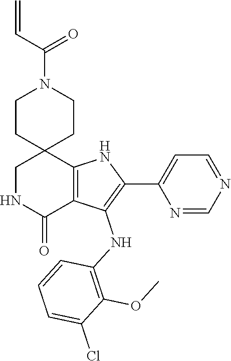 [SMILES: C=CC(=O)N1CCC2(CC1)CNC(=O)c1c2[nH]c(-c2ccncn2)c1Nc1cccc(Cl)c1OC] |
| 479 | 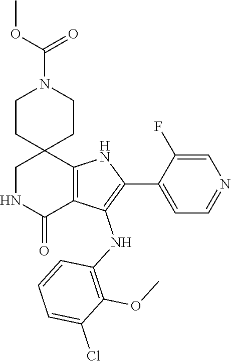 [SMILES: COC(=O)N1CCC2(CC1)CNC(=O)c1c2[nH]c(-c2ccncc2F)c1Nc1cccc(Cl)c1OC] |
| 480 | 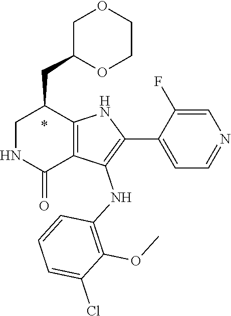 [SMILES: COc1c(Cl)cccc1Nc1c(-c2ccncc2F)[nH]c2c1C(=O)NC[C@H]2C[C@H]1COCCO1] |
| 659 | 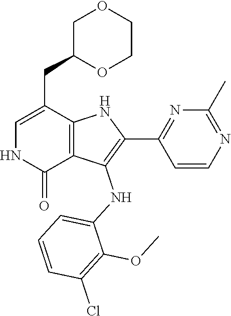 [SMILES: COc1c(Cl)cccc1Nc1c(-c2ccnc(C)n2)[nH]c2c(C[C@H]3COCCO3)c[nH]c(=O)c12] |
| 660 | 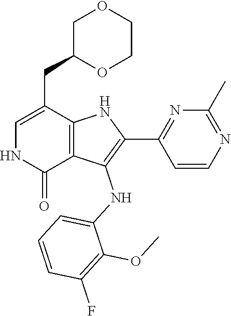 [SMILES: COc1c(F)cccc1Nc1c(-c2ccnc(C)n2)[nH]c2c(C[C@H]3COCCO3)c[nH]c(=O)c12] |
| 661 | 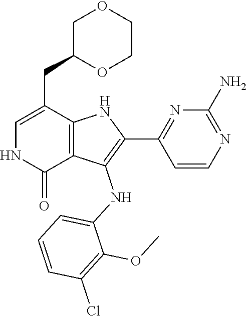 [SMILES: COc1c(Cl)cccc1Nc1c(-c2ccnc(N)n2)[nH]c2c(C[C@H]3COCCO3)c[nH]c(=O)c12] |
| 481 | 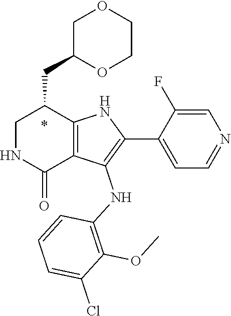 [SMILES: COc1c(Cl)cccc1Nc1c(-c2ccncc2F)[nH]c2c1C(=O)NC[C@@H]2C[C@H]1COCCO1] |
| 482 | 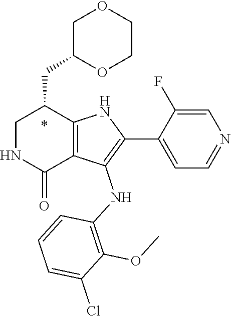 [SMILES: COc1c(Cl)cccc1Nc1c(-c2ccncc2F)[nH]c2c1C(=O)NC[C@@H]2C[C@@H]1COCCO1] |
| 483 | 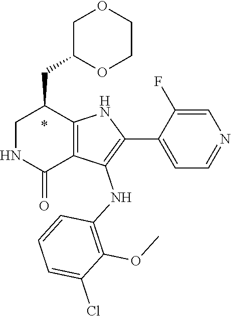 [SMILES: COc1c(Cl)cccc1Nc1c(-c2ccncc2F)[nH]c2c1C(=O)NC[C@H]2C[C@@H]1COCCO1] |
| 662 | 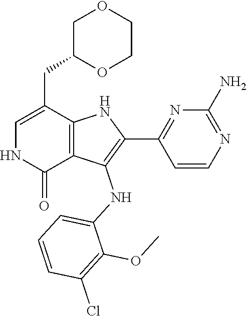 [SMILES: COc1c(Cl)cccc1Nc1c(-c2ccnc(N)n2)[nH]c2c(C[C@@H]3COCCO3)c[nH]c(=O)c12] |
| 663 | 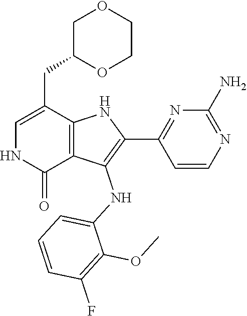 [SMILES: COc1c(F)cccc1Nc1c(-c2ccnc(N)n2)[nH]c2c(C[C@@H]3COCCO3)c[nH]c(=O)c12] |
| 664 | 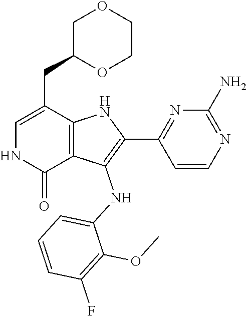 [SMILES: COc1c(F)cccc1Nc1c(-c2ccnc(N)n2)[nH]c2c(C[C@H]3COCCO3)c[nH]c(=O)c12] |
| 484 | 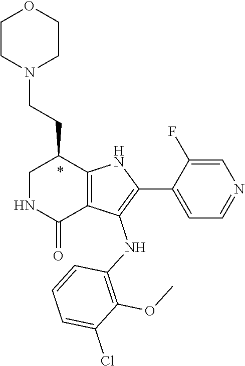 [SMILES: COc1c(Cl)cccc1Nc1c(-c2ccncc2F)[nH]c2c1C(=O)NC[C@H]2CCN1CCOCC1] |
| 485 | 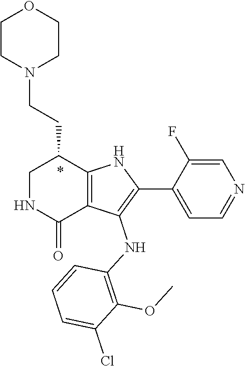 [SMILES: COc1c(Cl)cccc1Nc1c(-c2ccncc2F)[nH]c2c1C(=O)NC[C@@H]2CCN1CCOCC1] |
| 486 | 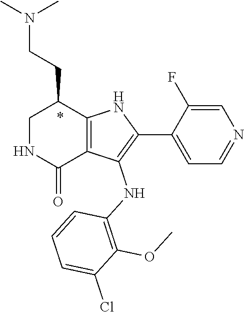 [SMILES: COc1c(Cl)cccc1Nc1c(-c2ccncc2F)[nH]c2c1C(=O)NC[C@H]2CCN(C)C] |
| 665 | 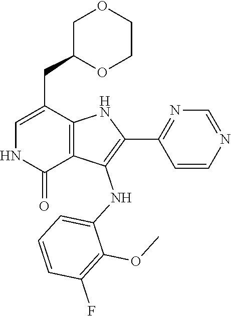 [SMILES: COc1c(F)cccc1Nc1c(-c2ccncn2)[nH]c2c(C[C@H]3COCCO3)c[nH]c(=O)c12] |
| 666 | 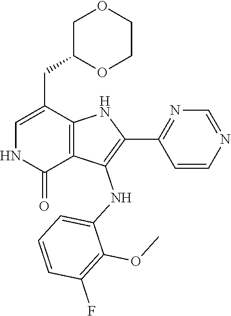 [SMILES: COc1c(F)cccc1Nc1c(-c2ccncn2)[nH]c2c(C[C@@H]3COCCO3)c[nH]c(=O)c12] |
| 667 | 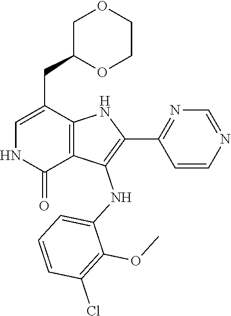 [SMILES: COc1c(Cl)cccc1Nc1c(-c2ccncn2)[nH]c2c(C[C@H]3COCCO3)c[nH]c(=O)c12] |
| 487 | 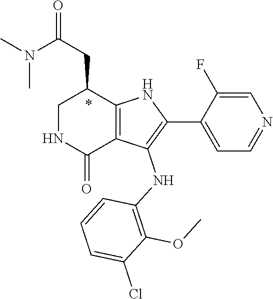 [SMILES: COc1c(Cl)cccc1Nc1c(-c2ccncc2F)[nH]c2c1C(=O)NC[C@H]2CC(=O)N(C)C] |
| 488 | 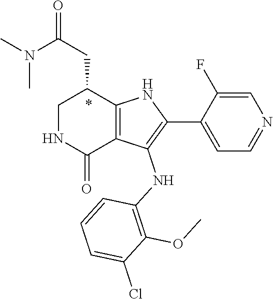 [SMILES: COc1c(Cl)cccc1Nc1c(-c2ccncc2F)[nH]c2c1C(=O)NC[C@@H]2CC(=O)N(C)C] |
| 489 | 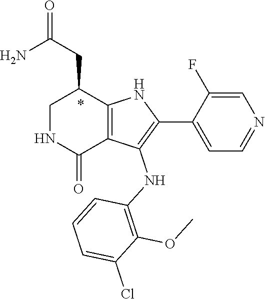 [SMILES: COc1c(Cl)cccc1Nc1c(-c2ccncc2F)[nH]c2c1C(=O)NC[C@H]2CC(N)=O] |
| 490 | 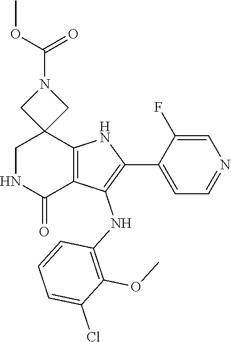 [SMILES: COC(=O)N1CC2(CNC(=O)c3c2[nH]c(-c2ccncc2F)c3Nc2cccc(Cl)c2OC)C1] |
| 668 | 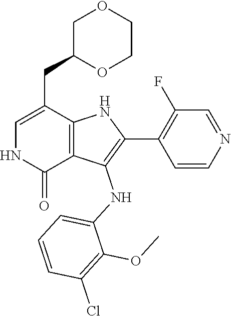 [SMILES: COc1c(Cl)cccc1Nc1c(-c2ccncc2F)[nH]c2c(C[C@H]3COCCO3)c[nH]c(=O)c12] |
| 669 | 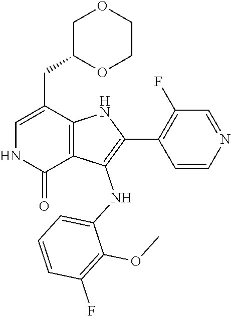 [SMILES: COc1c(F)cccc1Nc1c(-c2ccncc2F)[nH]c2c(C[C@@H]3COCCO3)c[nH]c(=O)c12] |
| 670 | 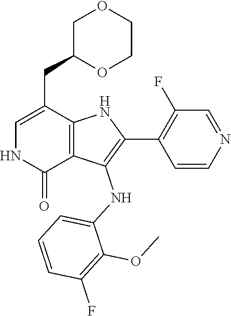 [SMILES: COc1c(F)cccc1Nc1c(-c2ccncc2F)[nH]c2c(C[C@H]3COCCO3)c[nH]c(=O)c12] |
| 671 | 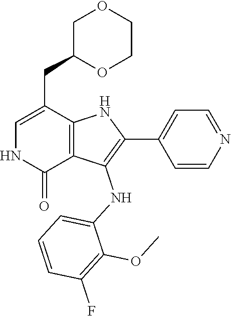 [SMILES: COc1c(F)cccc1Nc1c(-c2ccncc2)[nH]c2c(C[C@H]3COCCO3)c[nH]c(=O)c12] |
| 491 | 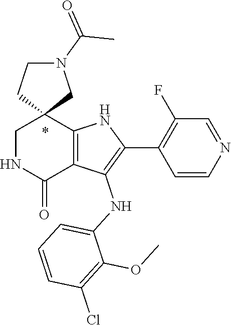 [SMILES: COc1c(Cl)cccc1Nc1c(-c2ccncc2F)[nH]c2c1C(=O)NC[C@]21CCN(C(C)=O)C1] |
| 492 | 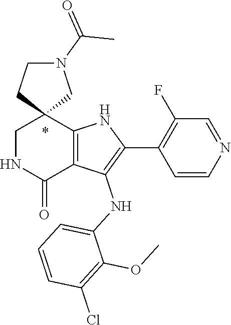 [SMILES: COc1c(Cl)cccc1Nc1c(-c2ccncc2F)[nH]c2c1C(=O)NC[C@@]21CCN(C(C)=O)C1] |
| 493 | 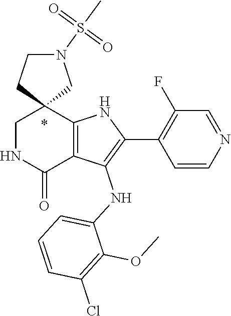 [SMILES: COc1c(Cl)cccc1Nc1c(-c2ccncc2F)[nH]c2c1C(=O)NC[C@@]21CCN(S(C)(=O)=O)C1] |
| 494 | 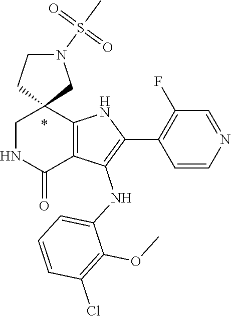 [SMILES: COc1c(Cl)cccc1Nc1c(-c2ccncc2F)[nH]c2c1C(=O)NC[C@]21CCN(S(C)(=O)=O)C1] |
| 672 | 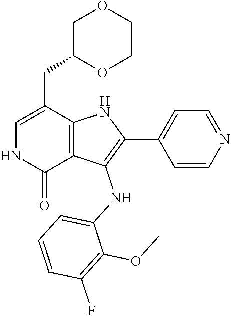 [SMILES: COc1c(F)cccc1Nc1c(-c2ccncc2)[nH]c2c(C[C@@H]3COCCO3)c[nH]c(=O)c12] |
| 673 | 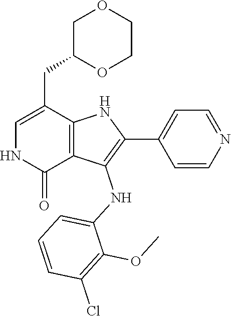 [SMILES: COc1c(Cl)cccc1Nc1c(-c2ccncc2)[nH]c2c(C[C@@H]3COCCO3)c[nH]c(=O)c12] |
| 674 | 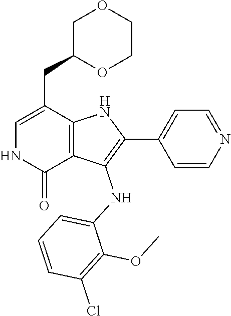 [SMILES: COc1c(Cl)cccc1Nc1c(-c2ccncc2)[nH]c2c(C[C@H]3COCCO3)c[nH]c(=O)c12] |
| 675 | 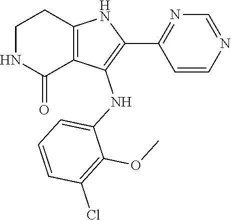 [SMILES: COc1c(Cl)cccc1Nc1c(-c2ccncn2)[nH]c2c1C(=O)NCC2] |
| 495 | 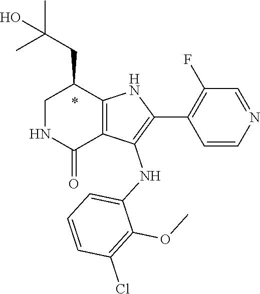 [SMILES: COc1c(Cl)cccc1Nc1c(-c2ccncc2F)[nH]c2c1C(=O)NC[C@H]2CC(C)(C)O] |
| 496 | 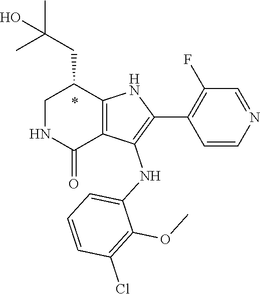 [SMILES: COc1c(Cl)cccc1Nc1c(-c2ccncc2F)[nH]c2c1C(=O)NC[C@@H]2CC(C)(C)O] |
| 497 | 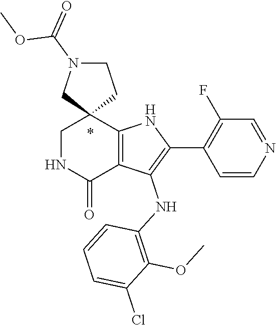 [SMILES: COC(=O)N1CC[C@]2(CNC(=O)c3c2[nH]c(-c2ccncc2F)c3Nc2cccc(Cl)c2OC)C1] |
| 498 | 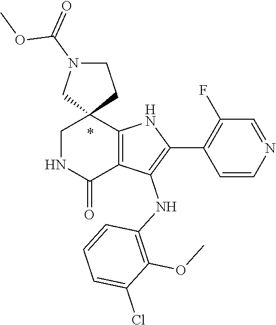 [SMILES: COC(=O)N1CC[C@@]2(CNC(=O)c3c2[nH]c(-c2ccncc2F)c3Nc2cccc(Cl)c2OC)C1] |
| 676 | 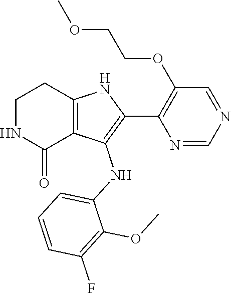 [SMILES: COCCOc1cncnc1-c1[nH]c2c(c1Nc1cccc(F)c1OC)C(=O)NCC2] |
| 677 | 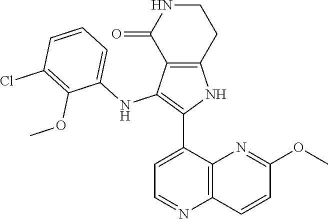 [SMILES: COc1ccc2nccc(-c3[nH]c4c(c3Nc3cccc(Cl)c3OC)C(=O)NCC4)c2n1] |
| 678 | |
| 679 | 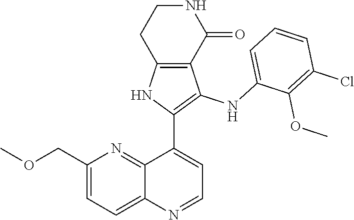 [SMILES: COCc1ccc2nccc(-c3[nH]c4c(c3Nc3cccc(Cl)c3OC)C(=O)NCC4)c2n1] |
| 499 | 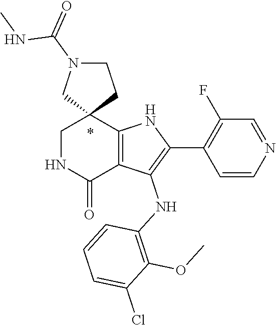 [SMILES: CNC(=O)N1CC[C@@]2(CNC(=O)c3c2[nH]c(-c2ccncc2F)c3Nc2cccc(Cl)c2OC)C1] |
| 500 | 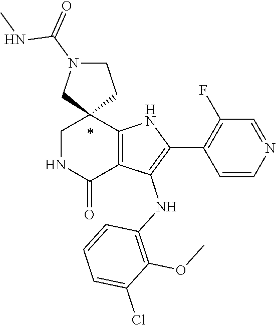 [SMILES: CNC(=O)N1CC[C@]2(CNC(=O)c3c2[nH]c(-c2ccncc2F)c3Nc2cccc(Cl)c2OC)C1] |
| 501 | 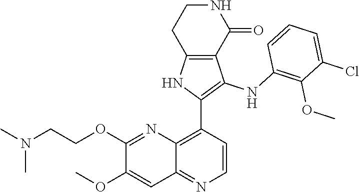 [SMILES: COc1cc2nccc(-c3[nH]c4c(c3Nc3cccc(Cl)c3OC)C(=O)NCC4)c2nc1OCCN(C)C] |
| 502 | 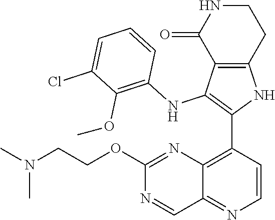 [SMILES: COc1c(Cl)cccc1Nc1c(-c2ccnc3cnc(OCCN(C)C)nc23)[nH]c2c1C(=O)NCC2] |
| 680 | 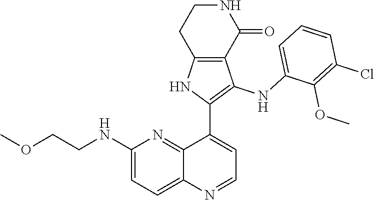 [SMILES: COCCNc1ccc2nccc(-c3[nH]c4c(c3Nc3cccc(Cl)c3OC)C(=O)NCC4)c2n1] |
| 681 | 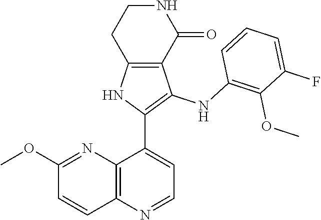 [SMILES: COc1ccc2nccc(-c3[nH]c4c(c3Nc3cccc(F)c3OC)C(=O)NCC4)c2n1] |
| 682 | 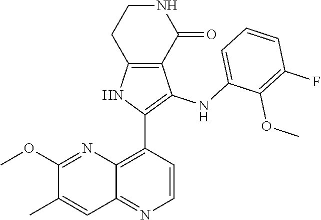 [SMILES: COc1nc2c(-c3[nH]c4c(c3Nc3cccc(F)c3OC)C(=O)NCC4)ccnc2cc1C] |
| 683 | 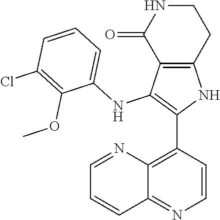 [SMILES: COc1c(Cl)cccc1Nc1c(-c2ccnc3cccnc23)[nH]c2c1C(=O)NCC2] |
| 503 | 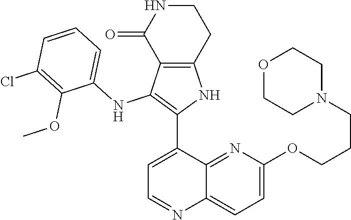 [SMILES: COc1c(Cl)cccc1Nc1c(-c2ccnc3ccc(OCCCN4CCOCC4)nc23)[nH]c2c1C(=O)NCC2] |
| 504 | 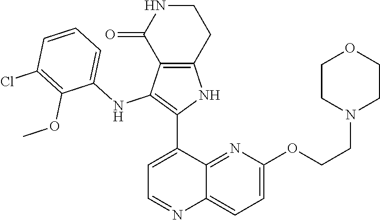 [SMILES: COc1c(Cl)cccc1Nc1c(-c2ccnc3ccc(OCCN4CCOCC4)nc23)[nH]c2c1C(=O)NCC2] |
| 505 | 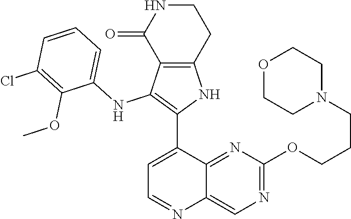 [SMILES: COc1c(Cl)cccc1Nc1c(-c2ccnc3cnc(OCCCN4CCOCC4)nc23)[nH]c2c1C(=O)NCC2] |
| 506 | 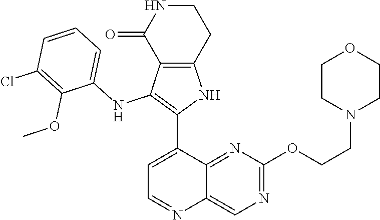 [SMILES: COc1c(Cl)cccc1Nc1c(-c2ccnc3cnc(OCCN4CCOCC4)nc23)[nH]c2c1C(=O)NCC2] |
| 507 | 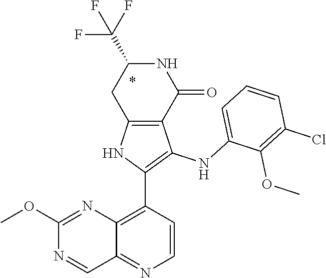 [SMILES: COc1ncc2nccc(-c3[nH]c4c(c3Nc3cccc(Cl)c3OC)C(=O)N[C@@H](C(F)(F)F)C4)c2n1] |
| 684 | 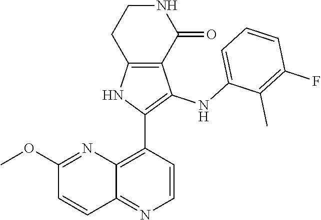 [SMILES: COc1ccc2nccc(-c3[nH]c4c(c3Nc3cccc(F)c3C)C(=O)NCC4)c2n1] |
| 685 | 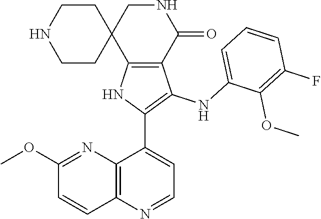 [SMILES: COc1ccc2nccc(-c3[nH]c4c(c3Nc3cccc(F)c3OC)C(=O)NCC43CCNCC3)c2n1] |
| 686 | 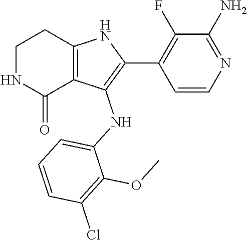 [SMILES: COc1c(Cl)cccc1Nc1c(-c2ccnc(N)c2F)[nH]c2c1C(=O)NCC2] |
| 687 | 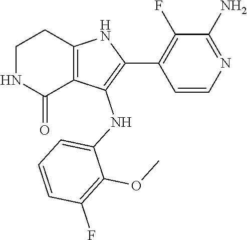 [SMILES: COc1c(F)cccc1Nc1c(-c2ccnc(N)c2F)[nH]c2c1C(=O)NCC2] |
| 688 | 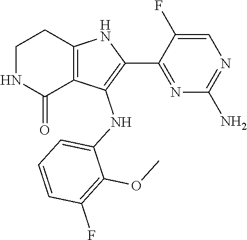 [SMILES: COc1c(F)cccc1Nc1c(-c2nc(N)ncc2F)[nH]c2c1C(=O)NCC2] |
| 508 | 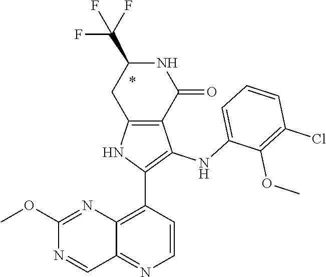 [SMILES: COc1ncc2nccc(-c3[nH]c4c(c3Nc3cccc(Cl)c3OC)C(=O)N[C@H](C(F)(F)F)C4)c2n1] |
| 509 | 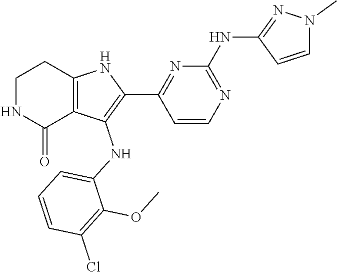 [SMILES: COc1c(Cl)cccc1Nc1c(-c2ccnc(Nc3ccn(C)n3)n2)[nH]c2c1C(=O)NCC2] |
| 510 | 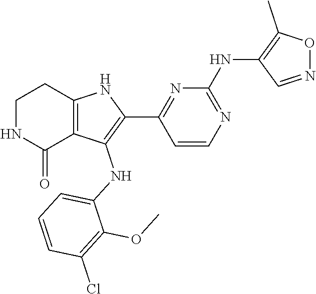 [SMILES: COc1c(Cl)cccc1Nc1c(-c2ccnc(Nc3cnoc3C)n2)[nH]c2c1C(=O)NCC2] |
| 511 | 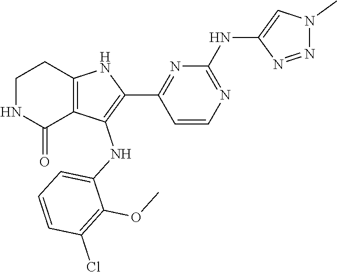 [SMILES: COc1c(Cl)cccc1Nc1c(-c2ccnc(Nc3cn(C)nn3)n2)[nH]c2c1C(=O)NCC2] |
| 689 | 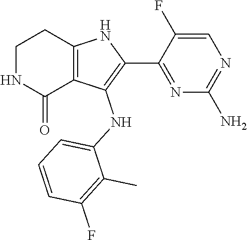 [SMILES: Cc1c(F)cccc1Nc1c(-c2nc(N)ncc2F)[nH]c2c1C(=O)NCC2] |
| 690 | 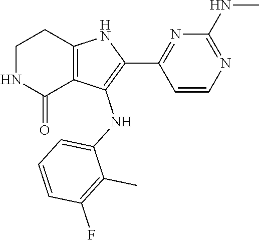 [SMILES: CNc1nccc(-c2[nH]c3c(c2Nc2cccc(F)c2C)C(=O)NCC3)n1] |
| 691 | 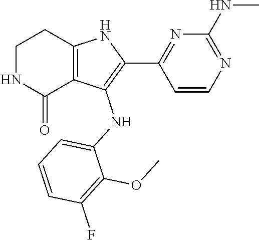 [SMILES: CNc1nccc(-c2[nH]c3c(c2Nc2cccc(F)c2OC)C(=O)NCC3)n1] |
| 692 | 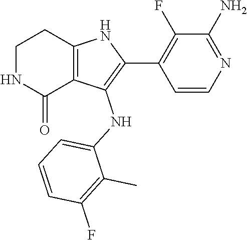 [SMILES: Cc1c(F)cccc1Nc1c(-c2ccnc(N)c2F)[nH]c2c1C(=O)NCC2] |
| 512 | 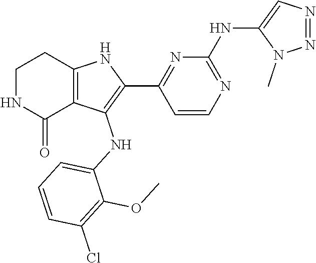 [SMILES: COc1c(Cl)cccc1Nc1c(-c2ccnc(Nc3cnnn3C)n2)[nH]c2c1C(=O)NCC2] |
| 513 | 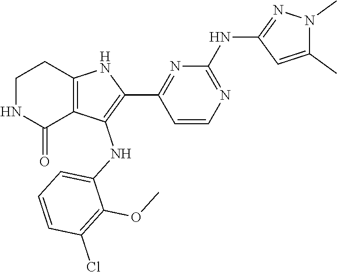 [SMILES: COc1c(Cl)cccc1Nc1c(-c2ccnc(Nc3cc(C)n(C)n3)n2)[nH]c2c1C(=O)NCC2] |
| 514 | 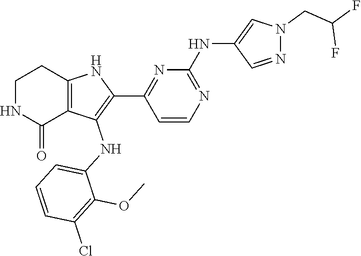 [SMILES: COc1c(Cl)cccc1Nc1c(-c2ccnc(Nc3cnn(CC(F)F)c3)n2)[nH]c2c1C(=O)NCC2] |
| 515 | 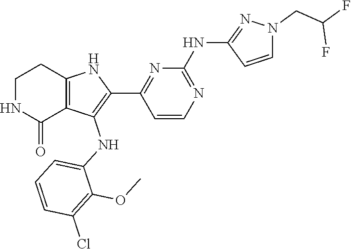 [SMILES: COc1c(Cl)cccc1Nc1c(-c2ccnc(Nc3ccn(CC(F)F)n3)n2)[nH]c2c1C(=O)NCC2] |
| 516 | 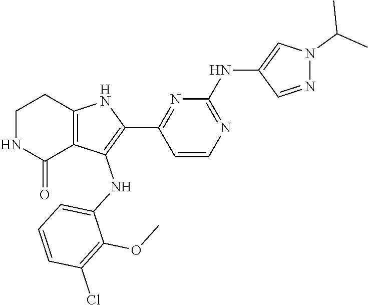 [SMILES: COc1c(Cl)cccc1Nc1c(-c2ccnc(Nc3cnn(C(C)C)c3)n2)[nH]c2c1C(=O)NCC2] |
| 693 | 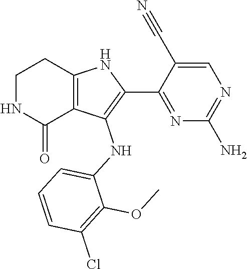 [SMILES: COc1c(Cl)cccc1Nc1c(-c2nc(N)ncc2C#N)[nH]c2c1C(=O)NCC2] |
| 694 | 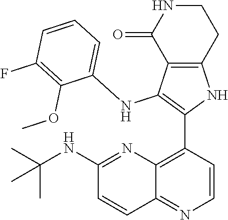 [SMILES: COc1c(F)cccc1Nc1c(-c2ccnc3ccc(NC(C)(C)C)nc23)[nH]c2c1C(=O)NCC2] |
| 695 | 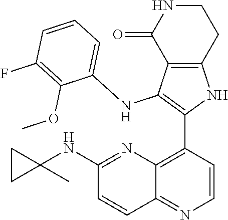 [SMILES: COc1c(F)cccc1Nc1c(-c2ccnc3ccc(NC4(C)CC4)nc23)[nH]c2c1C(=O)NCC2] |
| 696 | 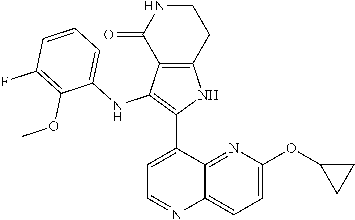 [SMILES: COc1c(F)cccc1Nc1c(-c2ccnc3ccc(OC4CC4)nc23)[nH]c2c1C(=O)NCC2] |
| 697 | 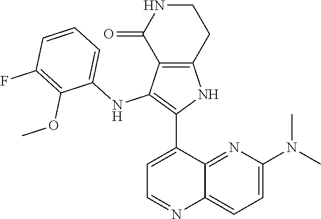 [SMILES: COc1c(F)cccc1Nc1c(-c2ccnc3ccc(N(C)C)nc23)[nH]c2c1C(=O)NCC2] |
| 517 | 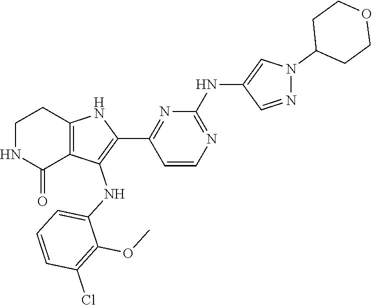 [SMILES: COc1c(Cl)cccc1Nc1c(-c2ccnc(Nc3cnn(C4CCOCC4)c3)n2)[nH]c2c1C(=O)NCC2] |
| 518 | 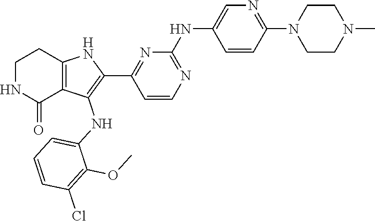 [SMILES: COc1c(Cl)cccc1Nc1c(-c2ccnc(Nc3ccc(N4CCN(C)CC4)nc3)n2)[nH]c2c1C(=O)NCC2] |
| 519 | 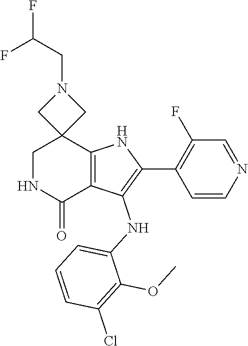 [SMILES: COc1c(Cl)cccc1Nc1c(-c2ccncc2F)[nH]c2c1C(=O)NCC21CN(CC(F)F)C1] |
| 520 | 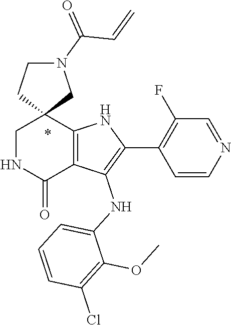 [SMILES: C=CC(=O)N1CC[C@]2(CNC(=O)c3c2[nH]c(-c2ccncc2F)c3Nc2cccc(Cl)c2OC)C1] |
| 698 | 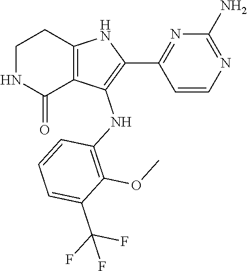 [SMILES: COc1c(Nc2c(-c3ccnc(N)n3)[nH]c3c2C(=O)NCC3)cccc1C(F)(F)F] |
| 699 | 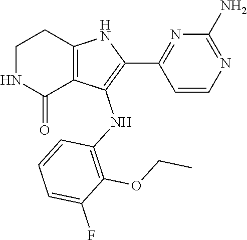 [SMILES: CCOc1c(F)cccc1Nc1c(-c2ccnc(N)n2)[nH]c2c1C(=O)NCC2] |
| 700 | 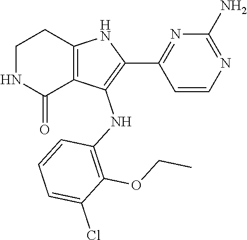 [SMILES: CCOc1c(Cl)cccc1Nc1c(-c2ccnc(N)n2)[nH]c2c1C(=O)NCC2] |
| 701 | 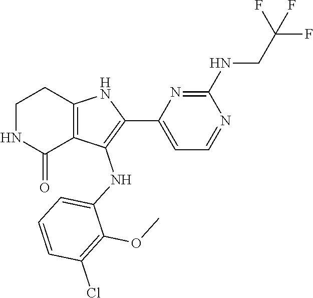 [SMILES: COc1c(Cl)cccc1Nc1c(-c2ccnc(NCC(F)(F)F)n2)[nH]c2c1C(=O)NCC2] |
| 521 | 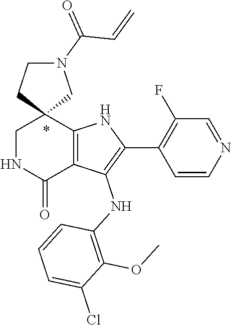 [SMILES: C=CC(=O)N1CC[C@@]2(CNC(=O)c3c2[nH]c(-c2ccncc2F)c3Nc2cccc(Cl)c2OC)C1] |
| 522 | 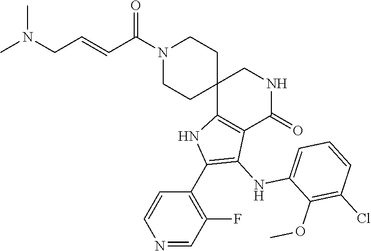 [SMILES: COc1c(Cl)cccc1Nc1c(-c2ccncc2F)[nH]c2c1C(=O)NCC21CCN(C(=O)/C=C/CN(C)C)CC1] |
| 523 | 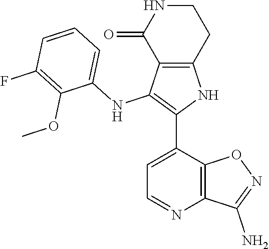 [SMILES: COc1c(F)cccc1Nc1c(-c2ccnc3c(N)noc23)[nH]c2c1C(=O)NCC2] |
| 524 | 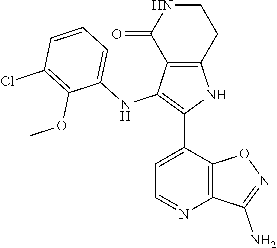 [SMILES: COc1c(Cl)cccc1Nc1c(-c2ccnc3c(N)noc23)[nH]c2c1C(=O)NCC2] |
| 702 | 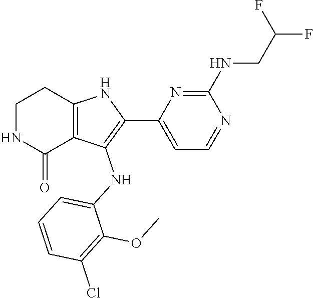 [SMILES: COc1c(Cl)cccc1Nc1c(-c2ccnc(NCC(F)F)n2)[nH]c2c1C(=O)NCC2] |
| 703 | 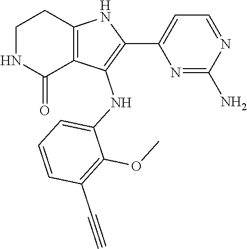 [SMILES: C#Cc1cccc(Nc2c(-c3ccnc(N)n3)[nH]c3c2C(=O)NCC3)c1OC] |
| 704 | 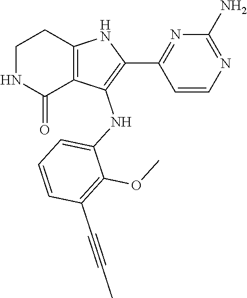 [SMILES: CC#Cc1cccc(Nc2c(-c3ccnc(N)n3)[nH]c3c2C(=O)NCC3)c1OC] |
| 705 | 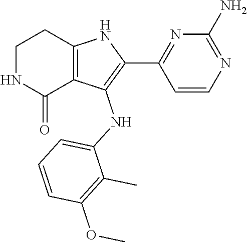 [SMILES: COc1cccc(Nc2c(-c3ccnc(N)n3)[nH]c3c2C(=O)NCC3)c1C] |
| 525 | 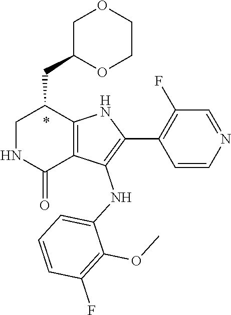 [SMILES: COc1c(F)cccc1Nc1c(-c2ccncc2F)[nH]c2c1C(=O)NC[C@@H]2C[C@H]1COCCO1] |
| 526 | 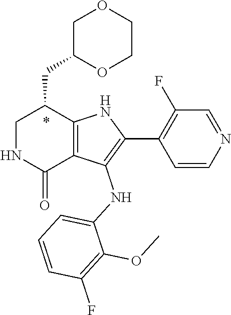 [SMILES: COc1c(F)cccc1Nc1c(-c2ccncc2F)[nH]c2c1C(=O)NC[C@@H]2C[C@@H]1COCCO1] |
| 527 | 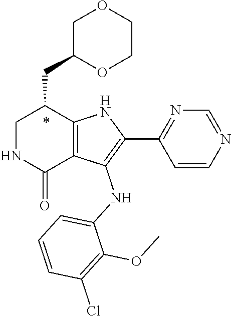 [SMILES: COc1c(Cl)cccc1Nc1c(-c2ccncn2)[nH]c2c1C(=O)NC[C@@H]2C[C@H]1COCCO1] |
| 528 | 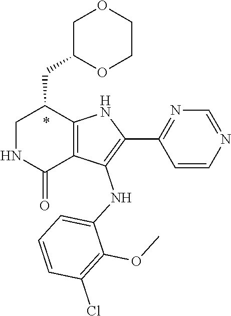 [SMILES: COc1c(Cl)cccc1Nc1c(-c2ccncn2)[nH]c2c1C(=O)NC[C@@H]2C[C@@H]1COCCO1] |
| 706 | 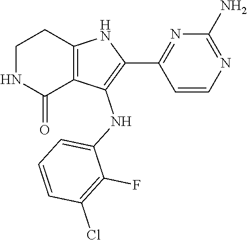 [SMILES: Nc1nccc(-c2[nH]c3c(c2Nc2cccc(Cl)c2F)C(=O)NCC3)n1] |
| 707 | 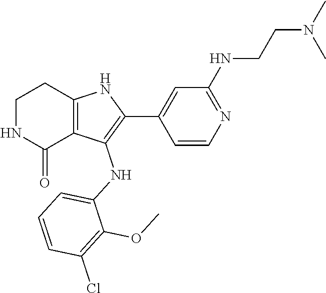 [SMILES: COc1c(Cl)cccc1Nc1c(-c2ccnc(NCCN(C)C)c2)[nH]c2c1C(=O)NCC2] |
| 708 | 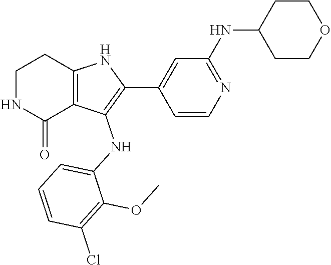 [SMILES: COc1c(Cl)cccc1Nc1c(-c2ccnc(NC3CCOCC3)c2)[nH]c2c1C(=O)NCC2] |
| 709 | 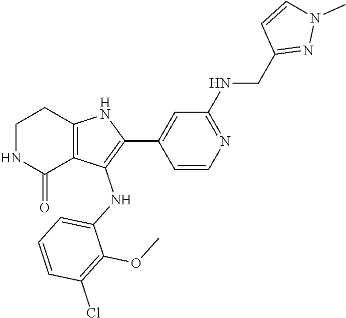 [SMILES: COc1c(Cl)cccc1Nc1c(-c2ccnc(NCc3ccn(C)n3)c2)[nH]c2c1C(=O)NCC2] |
| 529 | 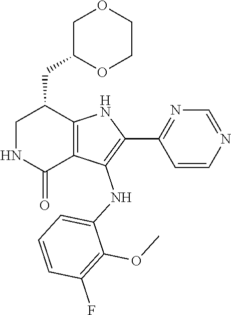 [SMILES: COc1c(F)cccc1Nc1c(-c2ccncn2)[nH]c2c1C(=O)NC[C@@H]2C[C@@H]1COCCO1] |
| 530 | 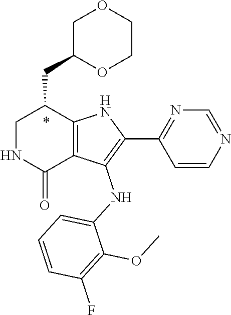 [SMILES: COc1c(F)cccc1Nc1c(-c2ccncn2)[nH]c2c1C(=O)NC[C@@H]2C[C@H]1COCCO1] |
| 531 | 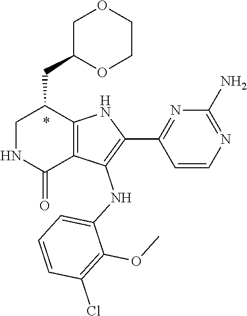 [SMILES: COc1c(Cl)cccc1Nc1c(-c2ccnc(N)n2)[nH]c2c1C(=O)NC[C@@H]2C[C@H]1COCCO1] |
| 532 | 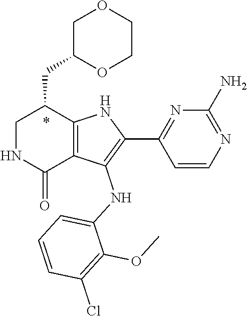 [SMILES: COc1c(Cl)cccc1Nc1c(-c2ccnc(N)n2)[nH]c2c1C(=O)NC[C@@H]2C[C@@H]1COCCO1] |
| 710 | 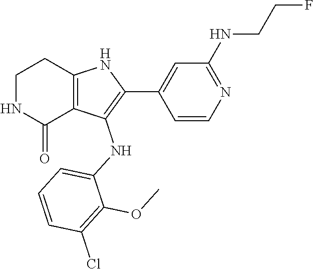 [SMILES: COc1c(Cl)cccc1Nc1c(-c2ccnc(NCCF)c2)[nH]c2c1C(=O)NCC2] |
| 711 | 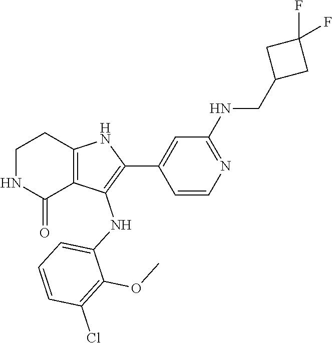 [SMILES: COc1c(Cl)cccc1Nc1c(-c2ccnc(NCC3CC(F)(F)C3)c2)[nH]c2c1C(=O)NCC2] |
| 712 | 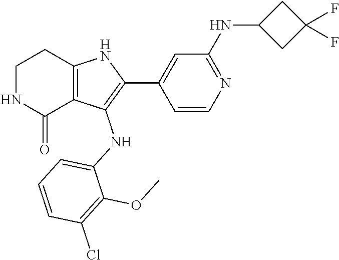 [SMILES: COc1c(Cl)cccc1Nc1c(-c2ccnc(NC3CC(F)(F)C3)c2)[nH]c2c1C(=O)NCC2] |
| 713 | 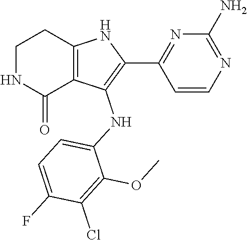 [SMILES: COc1c(Nc2c(-c3ccnc(N)n3)[nH]c3c2C(=O)NCC3)ccc(F)c1Cl] |
| 533 | 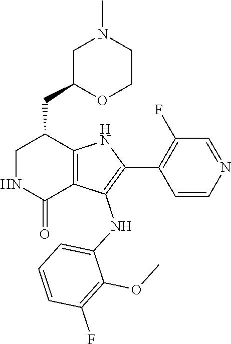 [SMILES: COc1c(F)cccc1Nc1c(-c2ccncc2F)[nH]c2c1C(=O)NC[C@@H]2C[C@H]1CN(C)CCO1] |
| 534 | 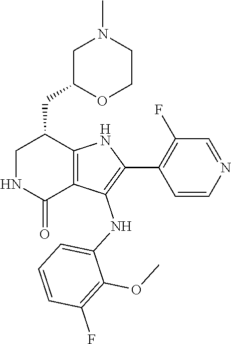 [SMILES: COc1c(F)cccc1Nc1c(-c2ccncc2F)[nH]c2c1C(=O)NC[C@@H]2C[C@@H]1CN(C)CCO1] |
| 535 | 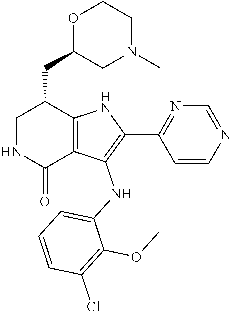 [SMILES: COc1c(Cl)cccc1Nc1c(-c2ccncn2)[nH]c2c1C(=O)NC[C@@H]2C[C@@H]1CN(C)CCO1] |
| 536 | 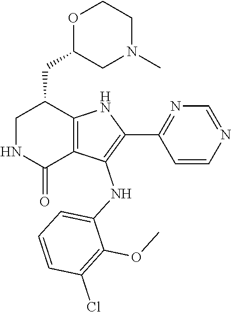 [SMILES: COc1c(Cl)cccc1Nc1c(-c2ccncn2)[nH]c2c1C(=O)NC[C@@H]2C[C@H]1CN(C)CCO1] |
| 714 | 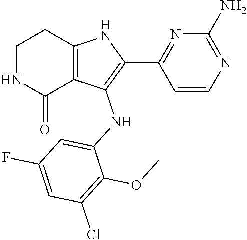 [SMILES: COc1c(Cl)cc(F)cc1Nc1c(-c2ccnc(N)n2)[nH]c2c1C(=O)NCC2] |
| 715 | 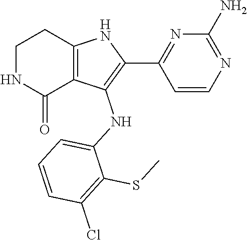 [SMILES: CSc1c(Cl)cccc1Nc1c(-c2ccnc(N)n2)[nH]c2c1C(=O)NCC2] |
| 716 | 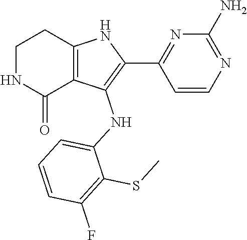 [SMILES: CSc1c(F)cccc1Nc1c(-c2ccnc(N)n2)[nH]c2c1C(=O)NCC2] |
| 717 | 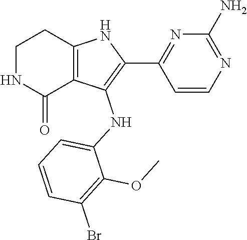 [SMILES: COc1c(Br)cccc1Nc1c(-c2ccnc(N)n2)[nH]c2c1C(=O)NCC2] |
| 537 | 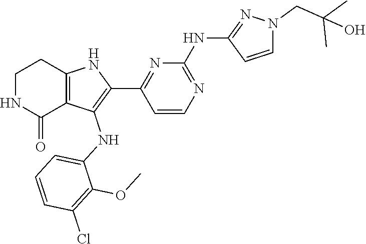 [SMILES: COc1c(Cl)cccc1Nc1c(-c2ccnc(Nc3ccn(CC(C)(C)O)n3)n2)[nH]c2c1C(=O)NCC2] |
| 538 | 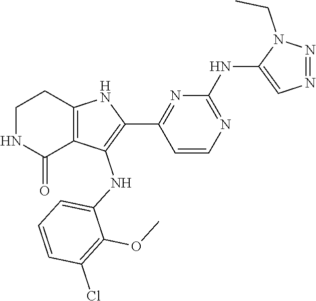 [SMILES: CCn1nncc1Nc1nccc(-c2[nH]c3c(c2Nc2cccc(Cl)c2OC)C(=O)NCC3)n1] |
| 539 | 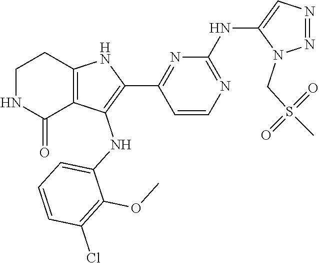 [SMILES: COc1c(Cl)cccc1Nc1c(-c2ccnc(Nc3cnnn3CS(C)(=O)=O)n2)[nH]c2c1C(=O)NCC2] |
| 540 | 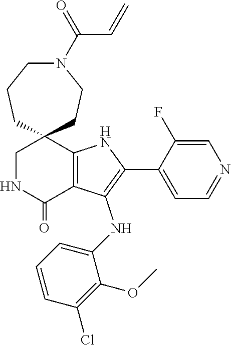 [SMILES: C=CC(=O)N1CCC[C@@]2(CC1)CNC(=O)c1c2[nH]c(-c2ccncc2F)c1Nc1cccc(Cl)c1OC] |
| 718 | 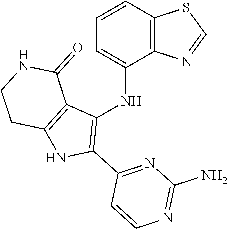 [SMILES: Nc1nccc(-c2[nH]c3c(c2Nc2cccc4scnc24)C(=O)NCC3)n1] |
| 719 | 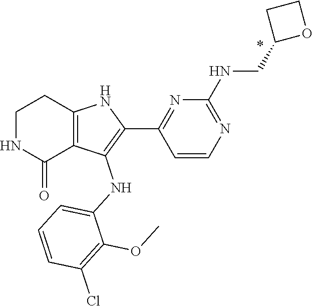 [SMILES: COc1c(Cl)cccc1Nc1c(-c2ccnc(NC[C@@H]3CCO3)n2)[nH]c2c1C(=O)NCC2] |
| 720 | 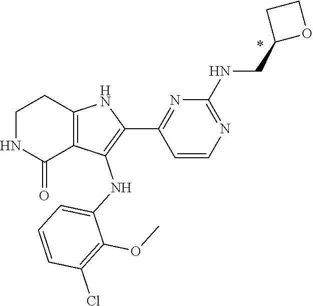 [SMILES: COc1c(Cl)cccc1Nc1c(-c2ccnc(NC[C@H]3CCO3)n2)[nH]c2c1C(=O)NCC2] |
| 721 | 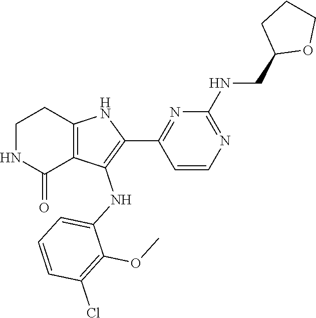 [SMILES: COc1c(Cl)cccc1Nc1c(-c2ccnc(NC[C@H]3CCCO3)n2)[nH]c2c1C(=O)NCC2] |
| 541 | 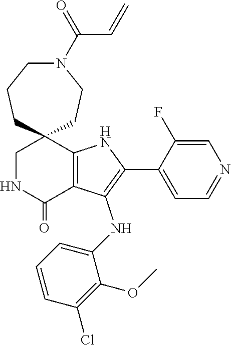 [SMILES: C=CC(=O)N1CCC[C@]2(CC1)CNC(=O)c1c2[nH]c(-c2ccncc2F)c1Nc1cccc(Cl)c1OC] |
| 542 | 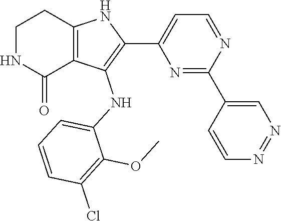 [SMILES: COc1c(Cl)cccc1Nc1c(-c2ccnc(-c3ccnnc3)n2)[nH]c2c1C(=O)NCC2] |
| 543 | 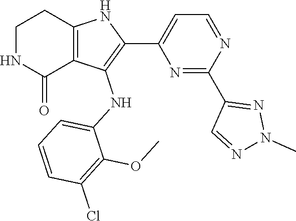 [SMILES: COc1c(Cl)cccc1Nc1c(-c2ccnc(-c3cnn(C)n3)n2)[nH]c2c1C(=O)NCC2] |
| 544 | 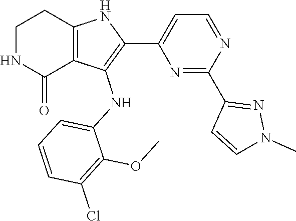 [SMILES: COc1c(Cl)cccc1Nc1c(-c2ccnc(-c3ccn(C)n3)n2)[nH]c2c1C(=O)NCC2] |
| 722 | 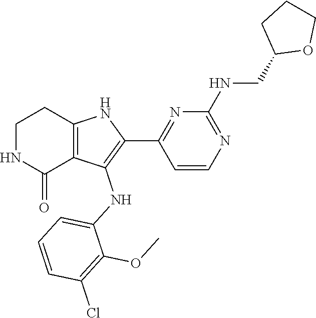 [SMILES: COc1c(Cl)cccc1Nc1c(-c2ccnc(NC[C@@H]3CCCO3)n2)[nH]c2c1C(=O)NCC2] |
| 723 | 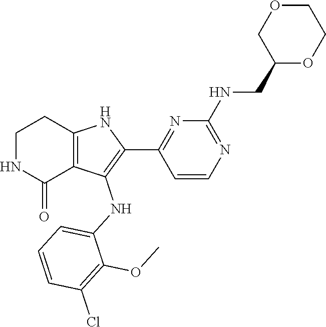 [SMILES: COc1c(Cl)cccc1Nc1c(-c2ccnc(NC[C@H]3COCCO3)n2)[nH]c2c1C(=O)NCC2] |
| 724 | 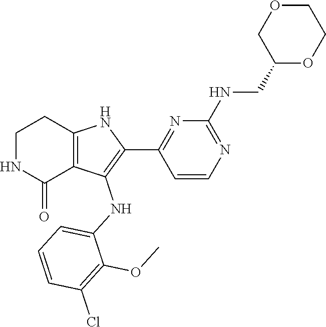 [SMILES: COc1c(Cl)cccc1Nc1c(-c2ccnc(NC[C@@H]3COCCO3)n2)[nH]c2c1C(=O)NCC2] |
| 725 | 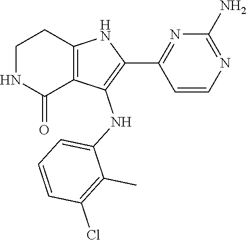 [SMILES: Cc1c(Cl)cccc1Nc1c(-c2ccnc(N)n2)[nH]c2c1C(=O)NCC2] |
| 545 | 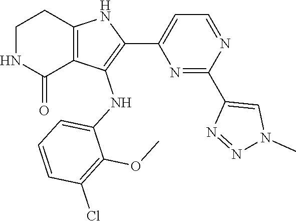 [SMILES: COc1c(Cl)cccc1Nc1c(-c2ccnc(-c3cn(C)nn3)n2)[nH]c2c1C(=O)NCC2] |
| 546 | 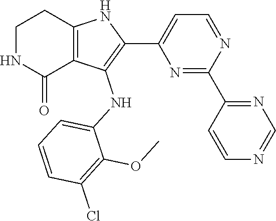 [SMILES: COc1c(Cl)cccc1Nc1c(-c2ccnc(-c3ccncn3)n2)[nH]c2c1C(=O)NCC2] |
| 547 | 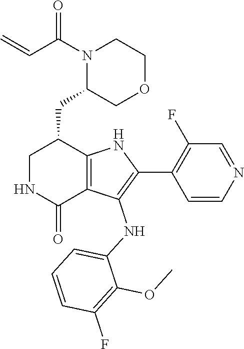 [SMILES: C=CC(=O)N1CCOC[C@@H]1C[C@H]1CNC(=O)c2c1[nH]c(-c1ccncc1F)c2Nc1cccc(F)c1OC] |
| 726 | 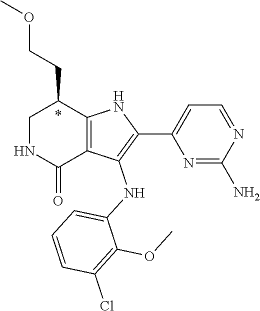 [SMILES: COCC[C@@H]1CNC(=O)c2c1[nH]c(-c1ccnc(N)n1)c2Nc1cccc(Cl)c1OC] |
| 727 | 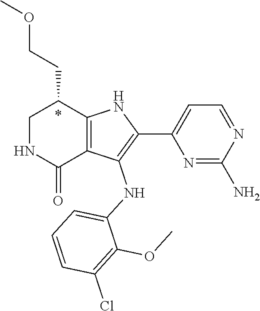 [SMILES: COCC[C@H]1CNC(=O)c2c1[nH]c(-c1ccnc(N)n1)c2Nc1cccc(Cl)c1OC] |
| 728 | 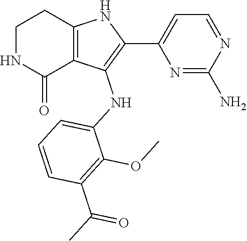 [SMILES: COc1c(Nc2c(-c3ccnc(N)n3)[nH]c3c2C(=O)NCC3)cccc1C(C)=O] |
| 548 | 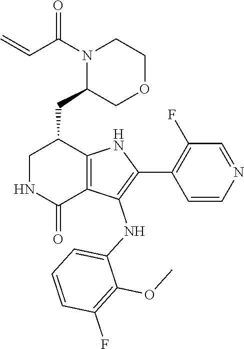 [SMILES: C=CC(=O)N1CCOC[C@H]1C[C@H]1CNC(=O)c2c1[nH]c(-c1ccncc1F)c2Nc1cccc(F)c1OC] |
| 549 | 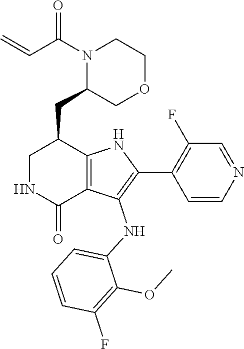 [SMILES: C=CC(=O)N1CCOC[C@H]1C[C@@H]1CNC(=O)c2c1[nH]c(-c1ccncc1F)c2Nc1cccc(F)c1OC] |
| 550 | 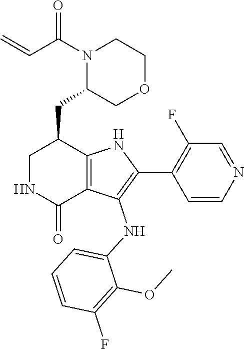 [SMILES: C=CC(=O)N1CCOC[C@@H]1C[C@@H]1CNC(=O)c2c1[nH]c(-c1ccncc1F)c2Nc1cccc(F)c1OC] |
| 729 | 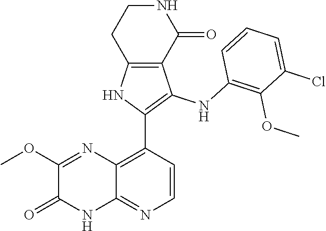 [SMILES: COc1c(Cl)cccc1Nc1c(-c2ccnc3[nH]c(=O)c(OC)nc23)[nH]c2c1C(=O)NCC2] |
| 730 | 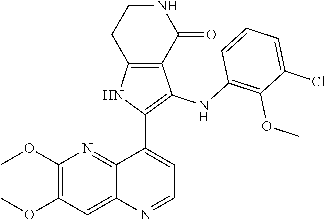 [SMILES: COc1cc2nccc(-c3[nH]c4c(c3Nc3cccc(Cl)c3OC)C(=O)NCC4)c2nc1OC] |
| 731 | 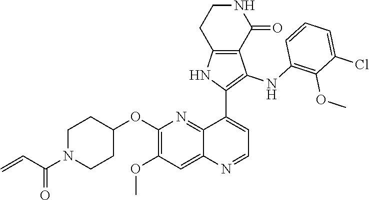 [SMILES: C=CC(=O)N1CCC(Oc2nc3c(-c4[nH]c5c(c4Nc4cccc(Cl)c4OC)C(=O)NCC5)ccnc3cc2OC)CC1] |
| 551 | 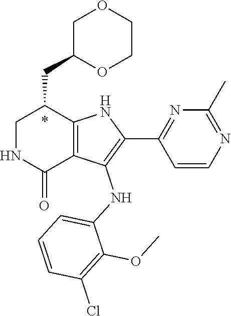 [SMILES: COc1c(Cl)cccc1Nc1c(-c2ccnc(C)n2)[nH]c2c1C(=O)NC[C@@H]2C[C@H]1COCCO1] |
| 552 | 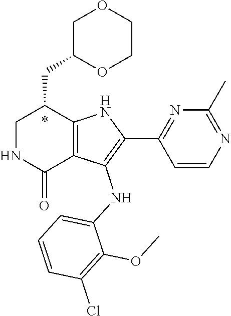 [SMILES: COc1c(Cl)cccc1Nc1c(-c2ccnc(C)n2)[nH]c2c1C(=O)NC[C@@H]2C[C@@H]1COCCO1] |
| 553 | 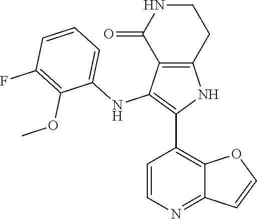 [SMILES: COc1c(F)cccc1Nc1c(-c2ccnc3ccoc23)[nH]c2c1C(=O)NCC2] |
| 554 | 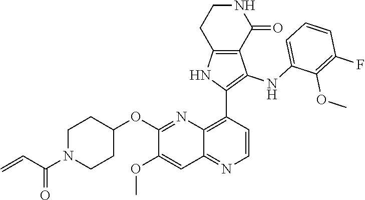 [SMILES: C=CC(=O)N1CCC(Oc2nc3c(-c4[nH]c5c(c4Nc4cccc(F)c4OC)C(=O)NCC5)ccnc3cc2OC)CC1] |
| 732 | 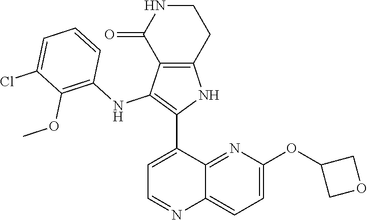 [SMILES: COc1c(Cl)cccc1Nc1c(-c2ccnc3ccc(OC4COC4)nc23)[nH]c2c1C(=O)NCC2] |
| 733 | 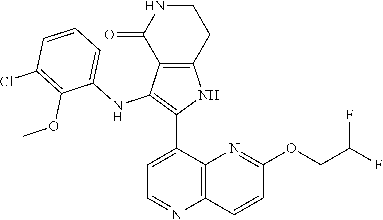 [SMILES: COc1c(Cl)cccc1Nc1c(-c2ccnc3ccc(OCC(F)F)nc23)[nH]c2c1C(=O)NCC2] |
| 734 | 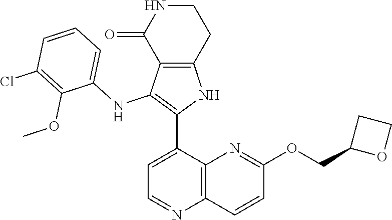 [SMILES: COc1c(Cl)cccc1Nc1c(-c2ccnc3ccc(OC[C@H]4CCO4)nc23)[nH]c2c1C(=O)NCC2] |
| 735 | 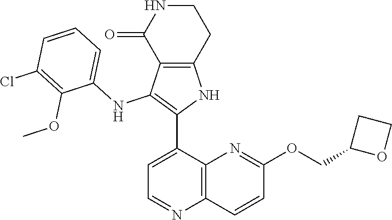 [SMILES: COc1c(Cl)cccc1Nc1c(-c2ccnc3ccc(OC[C@@H]4CCO4)nc23)[nH]c2c1C(=O)NCC2] |
| 555 | 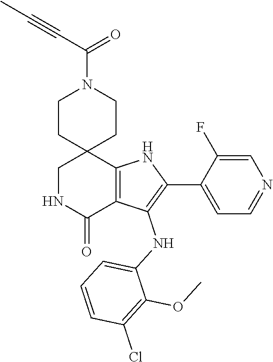 [SMILES: CC#CC(=O)N1CCC2(CC1)CNC(=O)c1c2[nH]c(-c2ccncc2F)c1Nc1cccc(Cl)c1OC] |
| 556 | 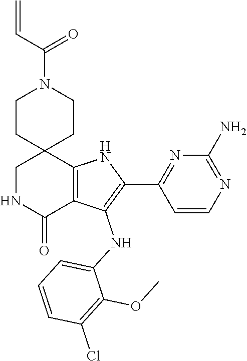 [SMILES: C=CC(=O)N1CCC2(CC1)CNC(=O)c1c2[nH]c(-c2ccnc(N)n2)c1Nc1cccc(Cl)c1OC] |
| 557 | 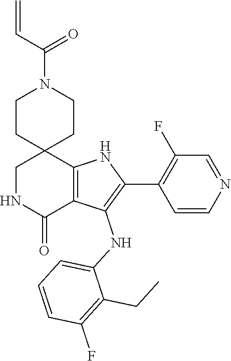 [SMILES: C=CC(=O)N1CCC2(CC1)CNC(=O)c1c2[nH]c(-c2ccncc2F)c1Nc1cccc(F)c1CC] |
| 736 | 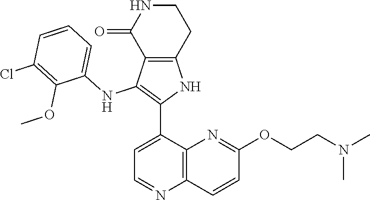 [SMILES: COc1c(Cl)cccc1Nc1c(-c2ccnc3ccc(OCCN(C)C)nc23)[nH]c2c1C(=O)NCC2] |
| 737 | 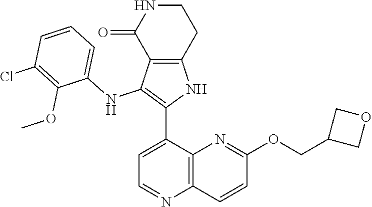 [SMILES: COc1c(Cl)cccc1Nc1c(-c2ccnc3ccc(OCC4COC4)nc23)[nH]c2c1C(=O)NCC2] |
| 738 | 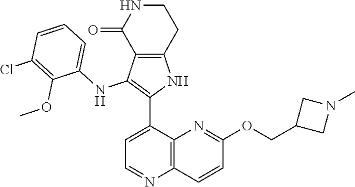 [SMILES: COc1c(Cl)cccc1Nc1c(-c2ccnc3ccc(OCC4CN(C)C4)nc23)[nH]c2c1C(=O)NCC2] |
| 558 | 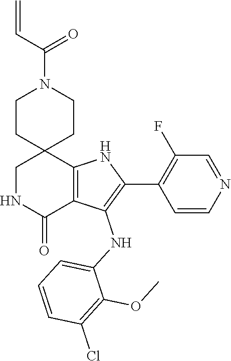 [SMILES: C=CC(=O)N1CCC2(CC1)CNC(=O)c1c2[nH]c(-c2ccncc2F)c1Nc1cccc(Cl)c1OC] |
| 559 | 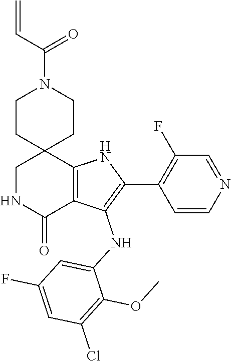 [SMILES: C=CC(=O)N1CCC2(CC1)CNC(=O)c1c2[nH]c(-c2ccncc2F)c1Nc1cc(F)cc(Cl)c1OC] |
| 560 | 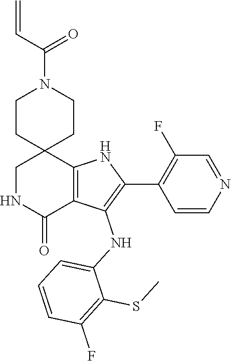 [SMILES: C=CC(=O)N1CCC2(CC1)CNC(=O)c1c2[nH]c(-c2ccncc2F)c1Nc1cccc(F)c1SC] |
| 739 | 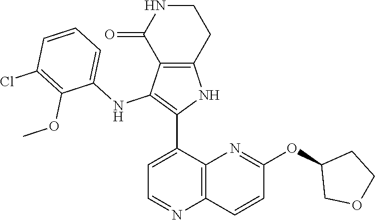 [SMILES: COc1c(Cl)cccc1Nc1c(-c2ccnc3ccc(O[C@H]4CCOC4)nc23)[nH]c2c1C(=O)NCC2] |
| 740 | 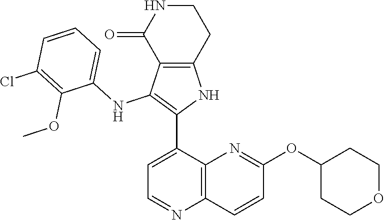 [SMILES: COc1c(Cl)cccc1Nc1c(-c2ccnc3ccc(OC4CCOCC4)nc23)[nH]c2c1C(=O)NCC2] |
| 741 | 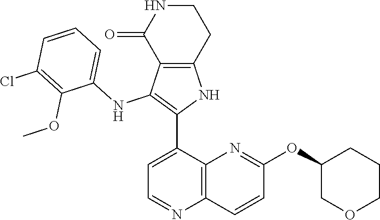 [SMILES: COc1c(Cl)cccc1Nc1c(-c2ccnc3ccc(O[C@H]4CCCOC4)nc23)[nH]c2c1C(=O)NCC2] |
| 561 | 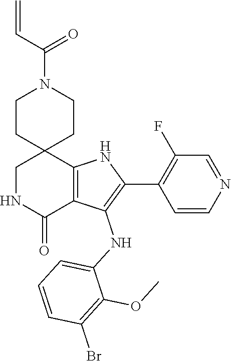 [SMILES: C=CC(=O)N1CCC2(CC1)CNC(=O)c1c2[nH]c(-c2ccncc2F)c1Nc1cccc(Br)c1OC] |
| 562 | 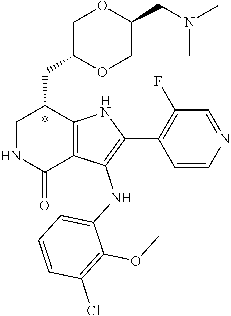 [SMILES: COc1c(Cl)cccc1Nc1c(-c2ccncc2F)[nH]c2c1C(=O)NC[C@@H]2C[C@@H]1CO[C@@H](CN(C)C)CO1] |
| 563 | 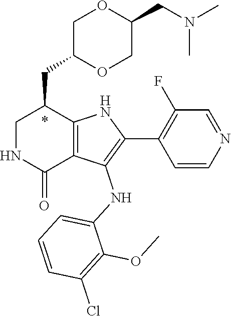 [SMILES: COc1c(Cl)cccc1Nc1c(-c2ccncc2F)[nH]c2c1C(=O)NC[C@H]2C[C@@H]1CO[C@@H](CN(C)C)CO1] |
| 742 | 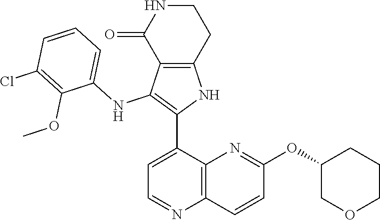 [SMILES: COc1c(Cl)cccc1Nc1c(-c2ccnc3ccc(O[C@@H]4CCCOC4)nc23)[nH]c2c1C(=O)NCC2] |
| 743 | 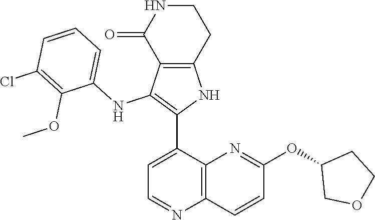 [SMILES: COc1c(Cl)cccc1Nc1c(-c2ccnc3ccc(O[C@@H]4CCOC4)nc23)[nH]c2c1C(=O)NCC2] |
| 744 | 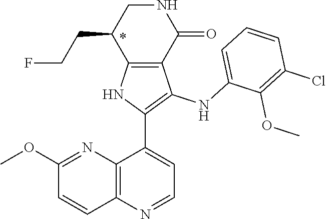 [SMILES: COc1ccc2nccc(-c3[nH]c4c(c3Nc3cccc(Cl)c3OC)C(=O)NC[C@@H]4CCF)c2n1] |
| 564 | 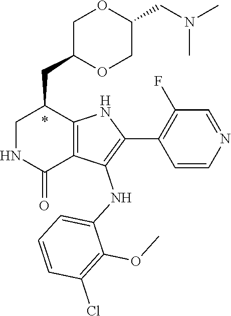 [SMILES: COc1c(Cl)cccc1Nc1c(-c2ccncc2F)[nH]c2c1C(=O)NC[C@H]2C[C@H]1CO[C@H](CN(C)C)CO1] |
| 565 | 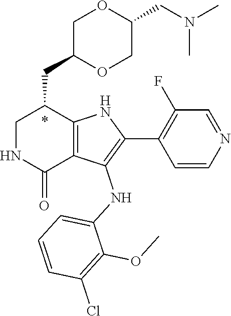 [SMILES: COc1c(Cl)cccc1Nc1c(-c2ccncc2F)[nH]c2c1C(=O)NC[C@@H]2C[C@H]1CO[C@H](CN(C)C)CO1] |
| 566 | 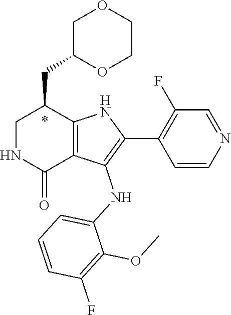 [SMILES: COc1c(F)cccc1Nc1c(-c2ccncc2F)[nH]c2c1C(=O)NC[C@H]2C[C@@H]1COCCO1] |
| 567 | 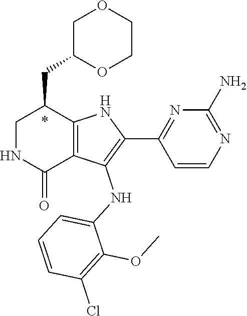 [SMILES: COc1c(Cl)cccc1Nc1c(-c2ccnc(N)n2)[nH]c2c1C(=O)NC[C@H]2C[C@@H]1COCCO1] |
| 745 | 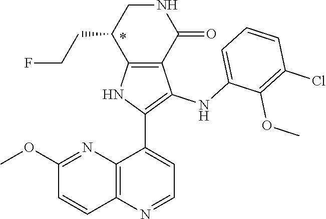 [SMILES: COc1ccc2nccc(-c3[nH]c4c(c3Nc3cccc(Cl)c3OC)C(=O)NC[C@H]4CCF)c2n1] |
| 746 | 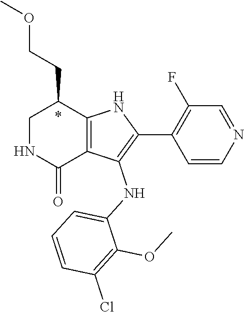 [SMILES: COCC[C@@H]1CNC(=O)c2c1[nH]c(-c1ccncc1F)c2Nc1cccc(Cl)c1OC] |
| 747 | 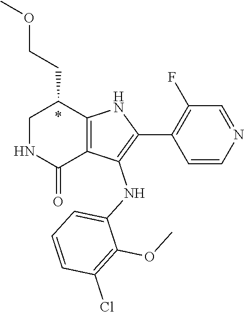 [SMILES: COCC[C@H]1CNC(=O)c2c1[nH]c(-c1ccncc1F)c2Nc1cccc(Cl)c1OC] |
| 748 | 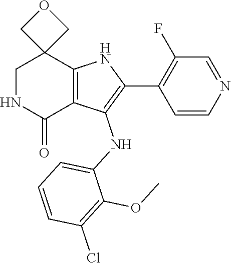 [SMILES: COc1c(Cl)cccc1Nc1c(-c2ccncc2F)[nH]c2c1C(=O)NCC21COC1] |
| 568 | 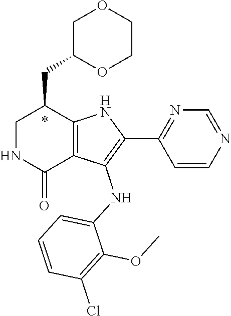 [SMILES: COc1c(Cl)cccc1Nc1c(-c2ccncn2)[nH]c2c1C(=O)NC[C@H]2C[C@@H]1COCCO1] |
| 569 | 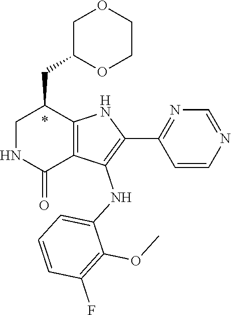 [SMILES: COc1c(F)cccc1Nc1c(-c2ccncn2)[nH]c2c1C(=O)NC[C@H]2C[C@@H]1COCCO1] |
| 570 | 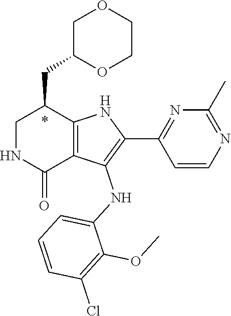 [SMILES: COc1c(Cl)cccc1Nc1c(-c2ccnc(C)n2)[nH]c2c1C(=O)NC[C@H]2C[C@@H]1COCCO1] |
| 571 | 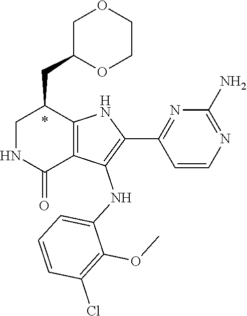 [SMILES: COc1c(Cl)cccc1Nc1c(-c2ccnc(N)n2)[nH]c2c1C(=O)NC[C@H]2C[C@H]1COCCO1] |
| 749 | 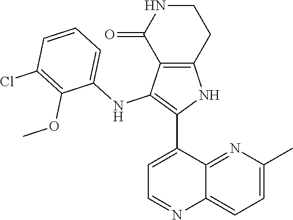 [SMILES: COc1c(Cl)cccc1Nc1c(-c2ccnc3ccc(C)nc23)[nH]c2c1C(=O)NCC2] |
| 750 | 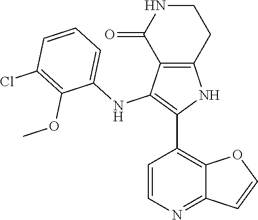 [SMILES: COc1c(Cl)cccc1Nc1c(-c2ccnc3ccoc23)[nH]c2c1C(=O)NCC2] |
| 751 | 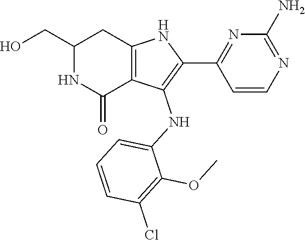 [SMILES: COc1c(Cl)cccc1Nc1c(-c2ccnc(N)n2)[nH]c2c1C(=O)NC(CO)C2] |
| 752 | 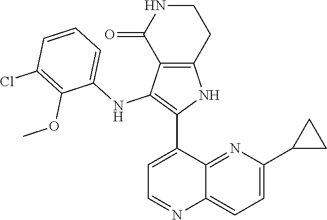 [SMILES: COc1c(Cl)cccc1Nc1c(-c2ccnc3ccc(C4CC4)nc23)[nH]c2c1C(=O)NCC2] |
| 572 | 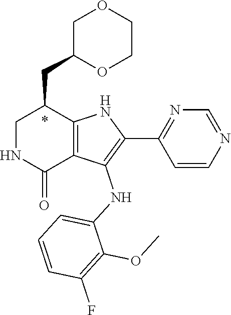 [SMILES: COc1c(F)cccc1Nc1c(-c2ccncn2)[nH]c2c1C(=O)NC[C@H]2C[C@H]1COCCO1] |
| 573 | 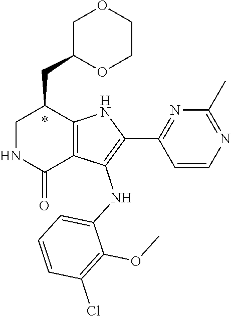 [SMILES: COc1c(Cl)cccc1Nc1c(-c2ccnc(C)n2)[nH]c2c1C(=O)NC[C@H]2C[C@H]1COCCO1] |
| 574 | 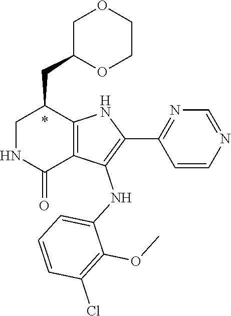 [SMILES: COc1c(Cl)cccc1Nc1c(-c2ccncn2)[nH]c2c1C(=O)NC[C@H]2C[C@H]1COCCO1] |
| 575 | 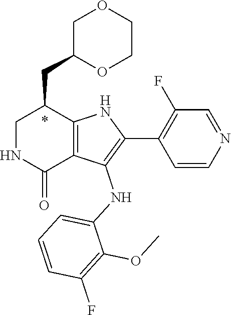 [SMILES: COc1c(F)cccc1Nc1c(-c2ccncc2F)[nH]c2c1C(=O)NC[C@H]2C[C@H]1COCCO1] |
| 753 | 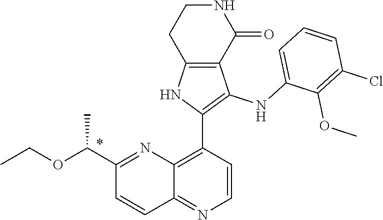 [SMILES: CCO[C@H](C)c1ccc2nccc(-c3[nH]c4c(c3Nc3cccc(Cl)c3OC)C(=O)NCC4)c2n1] |
| 754 | 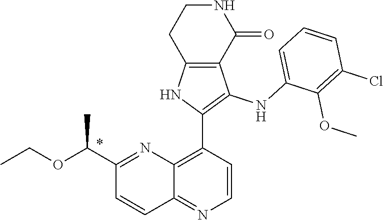 [SMILES: CCO[C@@H](C)c1ccc2nccc(-c3[nH]c4c(c3Nc3cccc(Cl)c3OC)C(=O)NCC4)c2n1] |
| 755 | 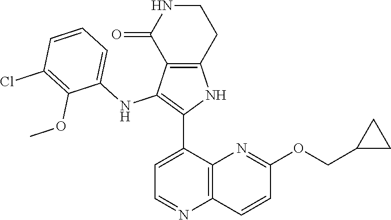 [SMILES: COc1c(Cl)cccc1Nc1c(-c2ccnc3ccc(OCC4CC4)nc23)[nH]c2c1C(=O)NCC2] |
| 756 | 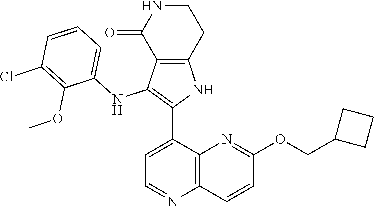 [SMILES: COc1c(Cl)cccc1Nc1c(-c2ccnc3ccc(OCC4CCC4)nc23)[nH]c2c1C(=O)NCC2] |
| 576 | 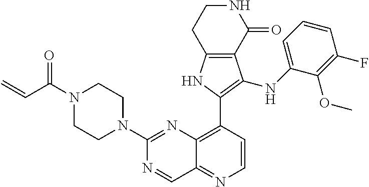 [SMILES: C=CC(=O)N1CCN(c2ncc3nccc(-c4[nH]c5c(c4Nc4cccc(F)c4OC)C(=O)NCC5)c3n2)CC1] |
| 577 | 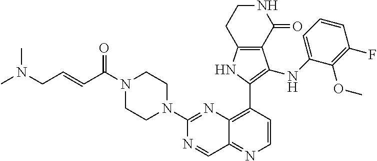 [SMILES: COc1c(F)cccc1Nc1c(-c2ccnc3cnc(N4CCN(C(=O)/C=C/CN(C)C)CC4)nc23)[nH]c2c1C(=O)NCC2] |
| 578 | 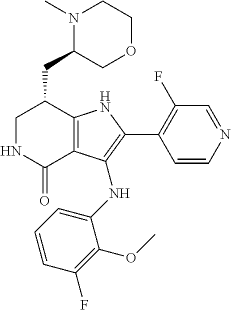 [SMILES: COc1c(F)cccc1Nc1c(-c2ccncc2F)[nH]c2c1C(=O)NC[C@@H]2C[C@@H]1COCCN1C] |
| 579 | 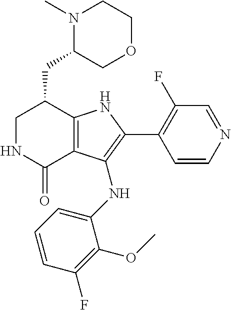 [SMILES: COc1c(F)cccc1Nc1c(-c2ccncc2F)[nH]c2c1C(=O)NC[C@@H]2C[C@H]1COCCN1C] |
| 757 | 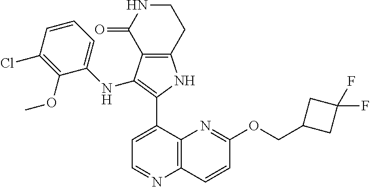 [SMILES: COc1c(Cl)cccc1Nc1c(-c2ccnc3ccc(OCC4CC(F)(F)C4)nc23)[nH]c2c1C(=O)NCC2] |
| 758 | 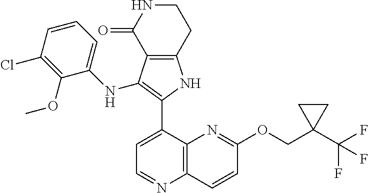 [SMILES: COc1c(Cl)cccc1Nc1c(-c2ccnc3ccc(OCC4(C(F)(F)F)CC4)nc23)[nH]c2c1C(=O)NCC2] |
| 759 | 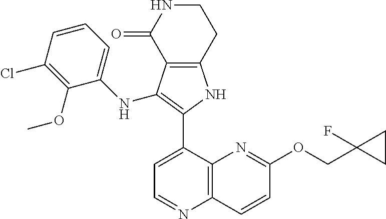 [SMILES: COc1c(Cl)cccc1Nc1c(-c2ccnc3ccc(OCC4(F)CC4)nc23)[nH]c2c1C(=O)NCC2] |
| 760 | 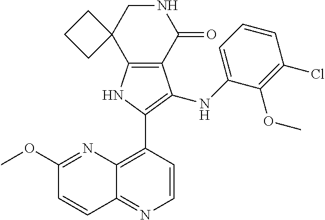 [SMILES: COc1ccc2nccc(-c3[nH]c4c(c3Nc3cccc(Cl)c3OC)C(=O)NCC43CCC3)c2n1] |
| 580 | 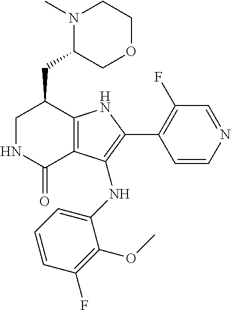 [SMILES: COc1c(F)cccc1Nc1c(-c2ccncc2F)[nH]c2c1C(=O)NC[C@H]2C[C@H]1COCCN1C] |
| 581 | 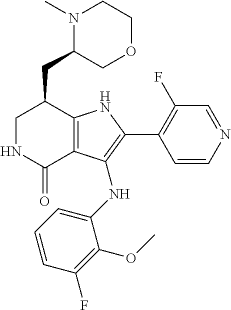 [SMILES: COc1c(F)cccc1Nc1c(-c2ccncc2F)[nH]c2c1C(=O)NC[C@H]2C[C@@H]1COCCN1C] |
| 582 | 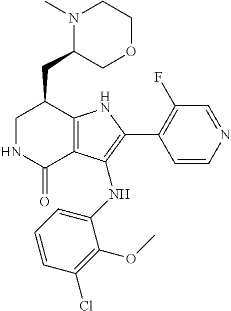 [SMILES: COc1c(Cl)cccc1Nc1c(-c2ccncc2F)[nH]c2c1C(=O)NC[C@H]2C[C@@H]1COCCN1C] |
| 583 | 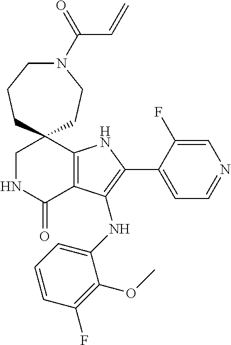 [SMILES: C=CC(=O)N1CCC[C@]2(CC1)CNC(=O)c1c2[nH]c(-c2ccncc2F)c1Nc1cccc(F)c1OC] |
| 761 | 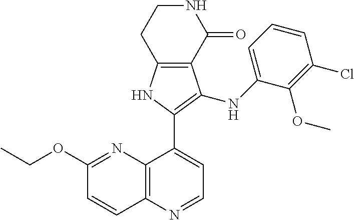 [SMILES: CCOc1ccc2nccc(-c3[nH]c4c(c3Nc3cccc(Cl)c3OC)C(=O)NCC4)c2n1] |
| 762 | 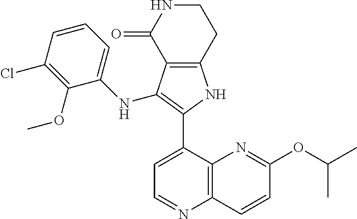 [SMILES: COc1c(Cl)cccc1Nc1c(-c2ccnc3ccc(OC(C)C)nc23)[nH]c2c1C(=O)NCC2] |
| 763 | 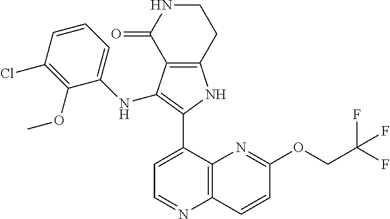 [SMILES: COc1c(Cl)cccc1Nc1c(-c2ccnc3ccc(OCC(F)(F)F)nc23)[nH]c2c1C(=O)NCC2] |
| 764 | 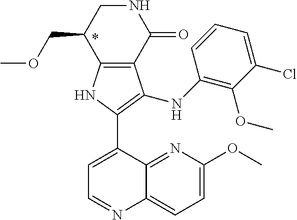 [SMILES: COC[C@H]1CNC(=O)c2c1[nH]c(-c1ccnc3ccc(OC)nc13)c2Nc1cccc(Cl)c1OC] |
| 584 | 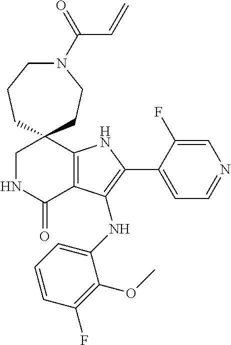 [SMILES: C=CC(=O)N1CCC[C@@]2(CC1)CNC(=O)c1c2[nH]c(-c2ccncc2F)c1Nc1cccc(F)c1OC] |
| 585 | 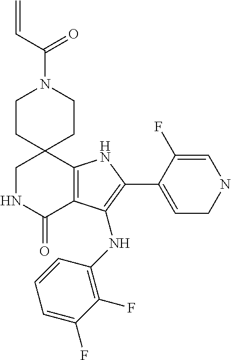 |
| 586 | 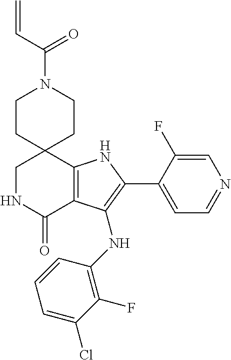 [SMILES: C=CC(=O)N1CCC2(CC1)CNC(=O)c1c2[nH]c(-c2ccncc2F)c1Nc1cccc(Cl)c1F] |
| 765 | 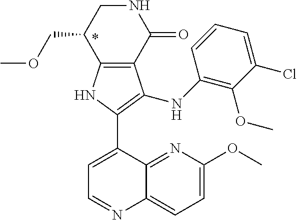 [SMILES: COC[C@@H]1CNC(=O)c2c1[nH]c(-c1ccnc3ccc(OC)nc13)c2Nc1cccc(Cl)c1OC] |
| 766 | 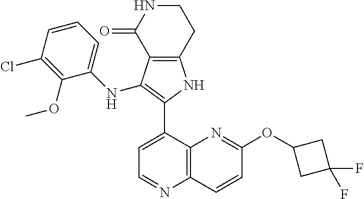 [SMILES: COc1c(Cl)cccc1Nc1c(-c2ccnc3ccc(OC4CC(F)(F)C4)nc23)[nH]c2c1C(=O)NCC2] |
| 767 | 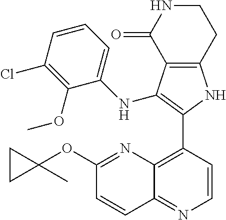 [SMILES: COc1c(Cl)cccc1Nc1c(-c2ccnc3ccc(OC4(C)CC4)nc23)[nH]c2c1C(=O)NCC2] |
| 587 | 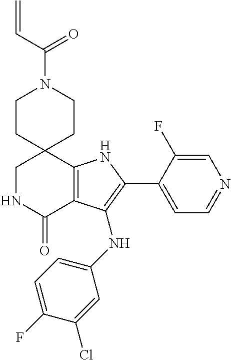 [SMILES: C=CC(=O)N1CCC2(CC1)CNC(=O)c1c2[nH]c(-c2ccncc2F)c1Nc1ccc(F)c(Cl)c1] |
| 588 | 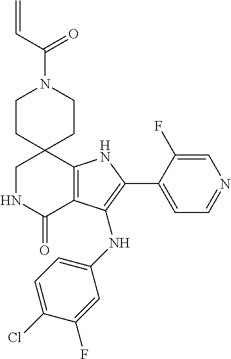 [SMILES: C=CC(=O)N1CCC2(CC1)CNC(=O)c1c2[nH]c(-c2ccncc2F)c1Nc1ccc(Cl)c(F)c1] |
| 589 | 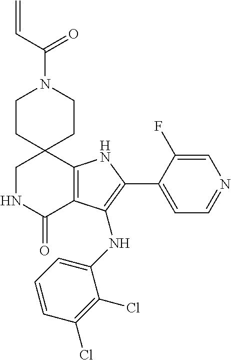 [SMILES: C=CC(=O)N1CCC2(CC1)CNC(=O)c1c2[nH]c(-c2ccncc2F)c1Nc1cccc(Cl)c1Cl] |
| 768 | 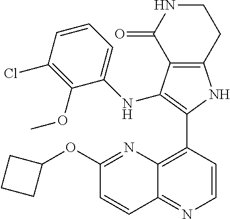 [SMILES: COc1c(Cl)cccc1Nc1c(-c2ccnc3ccc(OC4CCC4)nc23)[nH]c2c1C(=O)NCC2] |
| 769 | 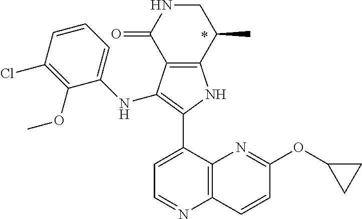 [SMILES: COc1c(Cl)cccc1Nc1c(-c2ccnc3ccc(OC4CC4)nc23)[nH]c2c1C(=O)NC[C@H]2C] |
| 770 | 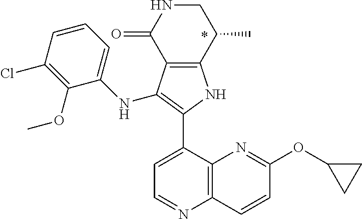 [SMILES: COc1c(Cl)cccc1Nc1c(-c2ccnc3ccc(OC4CC4)nc23)[nH]c2c1C(=O)NC[C@@H]2C] |
| 590 | 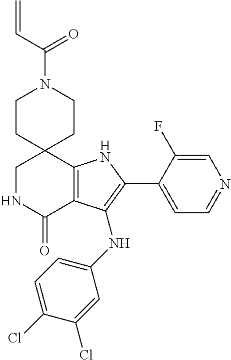 [SMILES: C=CC(=O)N1CCC2(CC1)CNC(=O)c1c2[nH]c(-c2ccncc2F)c1Nc1ccc(Cl)c(Cl)c1] |
| 591 | 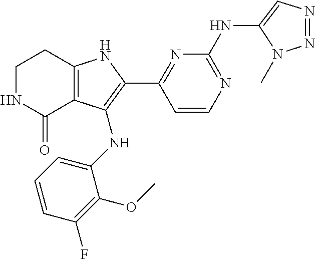 |
| 592 | 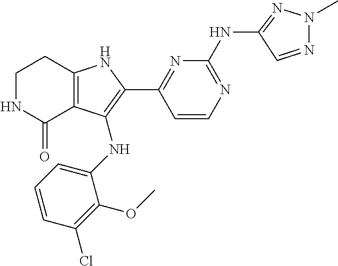 |
| 593 | 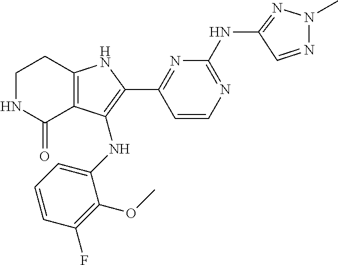 |
| 771 | 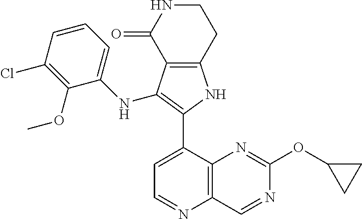 [SMILES: COc1c(Cl)cccc1Nc1c(-c2ccnc3cnc(OC4CC4)nc23)[nH]c2c1C(=O)NCC2] |
| 772 | 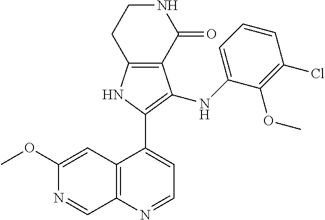 [SMILES: COc1cc2c(-c3[nH]c4c(c3Nc3cccc(Cl)c3OC)C(=O)NCC4)ccnc2cn1] |
| 773 | 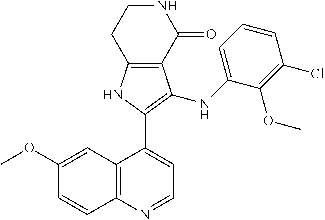 [SMILES: COc1ccc2nccc(-c3[nH]c4c(c3Nc3cccc(Cl)c3OC)C(=O)NCC4)c2c1] |
| 774 | 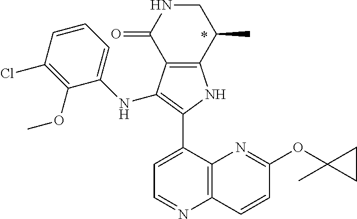 [SMILES: COc1c(Cl)cccc1Nc1c(-c2ccnc3ccc(OC4(C)CC4)nc23)[nH]c2c1C(=O)NC[C@H]2C] |
| 594 | 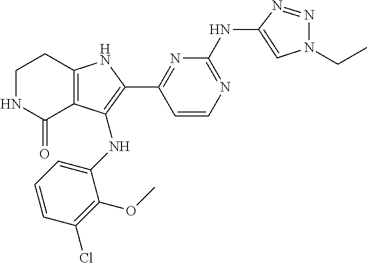 |
| 595 | 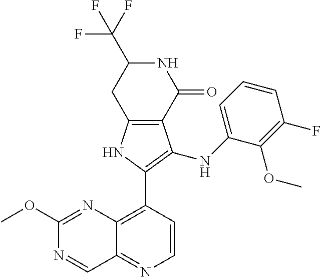 [SMILES: COc1ncc2nccc(-c3[nH]c4c(c3Nc3cccc(F)c3OC)C(=O)NC(C(F)(F)F)C4)c2n1] |
| 596 | 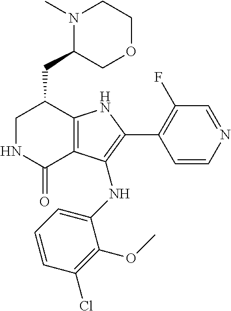 |
| 597 | 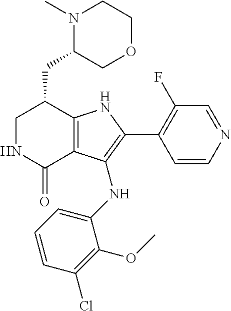 |
| 775 | 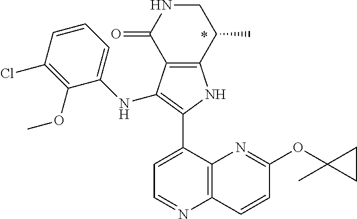 [SMILES: COc1c(Cl)cccc1Nc1c(-c2ccnc3ccc(OC4(C)CC4)nc23)[nH]c2c1C(=O)NC[C@@H]2C] |
| 776 | 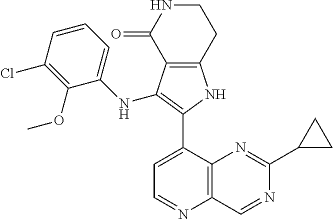 [SMILES: COc1c(Cl)cccc1Nc1c(-c2ccnc3cnc(C4CC4)nc23)[nH]c2c1C(=O)NCC2] |
| 777 | 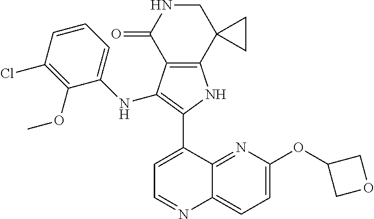 [SMILES: COc1c(Cl)cccc1Nc1c(-c2ccnc3ccc(OC4COC4)nc23)[nH]c2c1C(=O)NCC21CC1] |
| 778 | 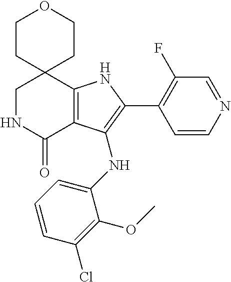 |
| 598 | 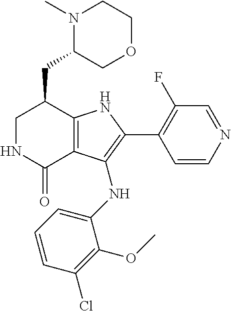 |
| 599 | 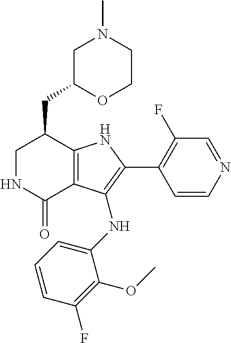 |
| 600 | 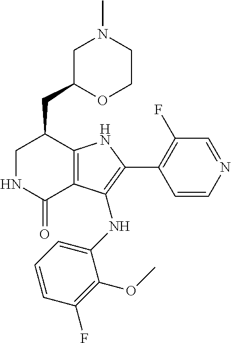 |
| 601 | 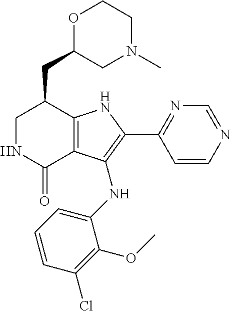 |
| 779 | 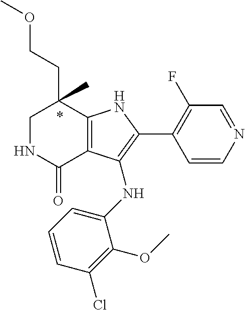 |
| 780 | 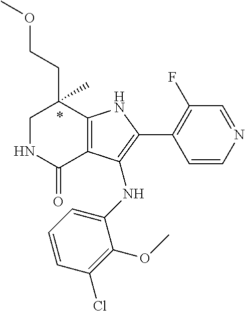 |
| 781 | 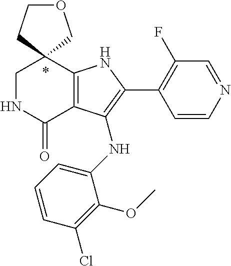 |
| 782 | 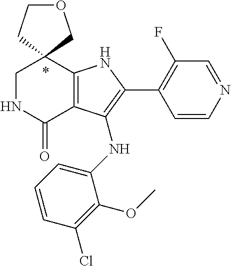 |
| 602 | 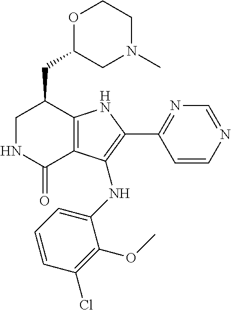 |
| 603 | 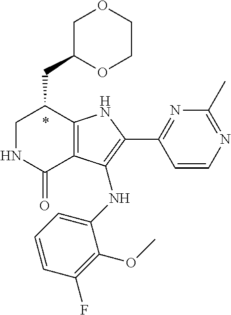 |
| 604 | 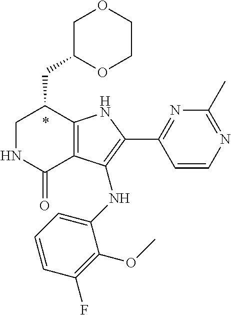 |
| 605 | 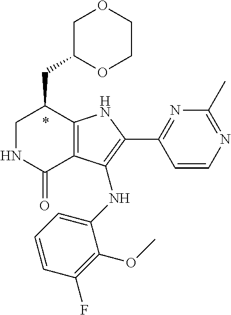 |
| 783 | 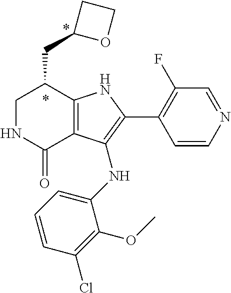 |
| 784 | 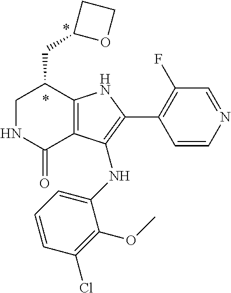 |
| 785 | 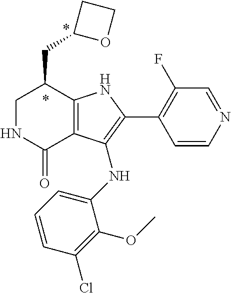 |
| 786 | 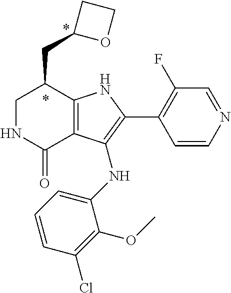 |
| 606 | 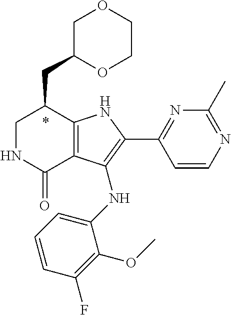 |
| 607 | 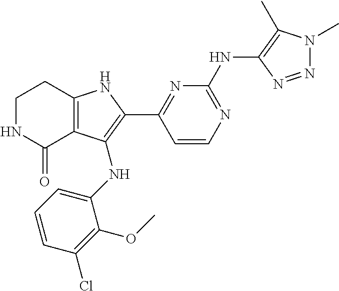 |
| 608 | 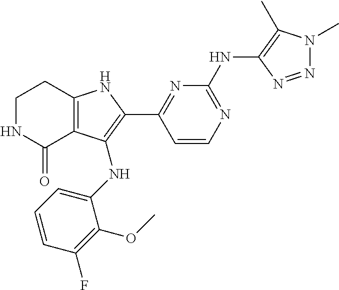 |
| 609 | 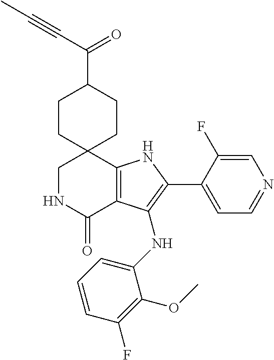 |
| 787 | 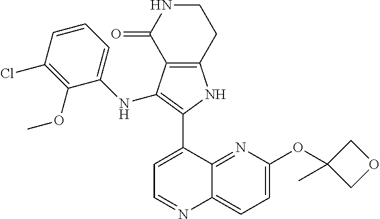 [SMILES: COc1c(Cl)cccc1Nc1c(-c2ccnc3ccc(OC4(C)COC4)nc23)[nH]c2c1C(=O)NCC2] |
| 788 | 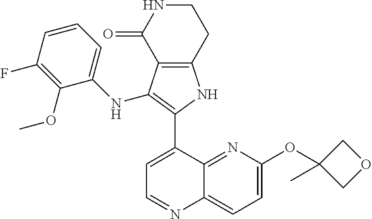 [SMILES: COc1c(F)cccc1Nc1c(-c2ccnc3ccc(OC4(C)COC4)nc23)[nH]c2c1C(=O)NCC2] |
| 789 | 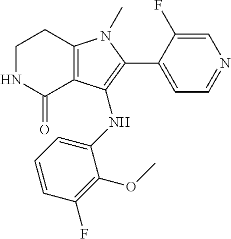 |
| 790 | 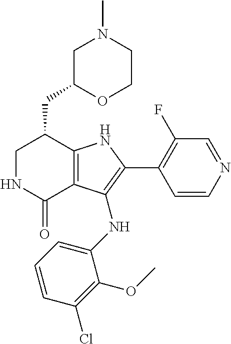 |
| 610 | 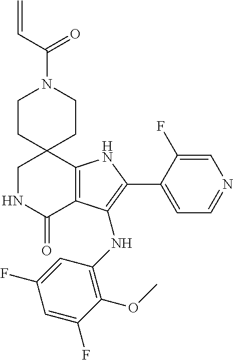 |
| 611 | 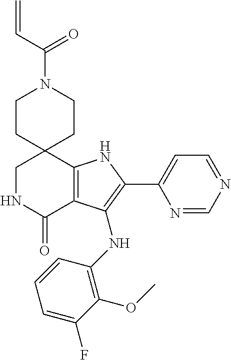 |
| 612 | 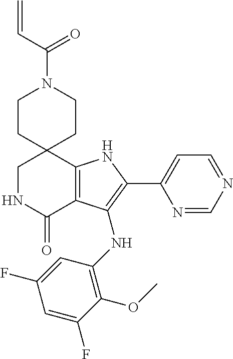 |
| 791 | 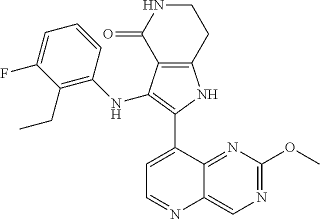 [SMILES: CCc1c(F)cccc1Nc1c(-c2ccnc3cnc(OC)nc23)[nH]c2c1C(=O)NCC2] |
| 792 | 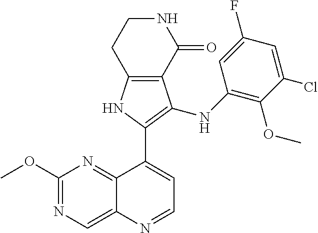 [SMILES: COc1ncc2nccc(-c3[nH]c4c(c3Nc3cc(F)cc(Cl)c3OC)C(=O)NCC4)c2n1] |
| 793 | 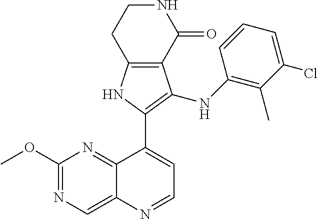 [SMILES: COc1ncc2nccc(-c3[nH]c4c(c3Nc3cccc(Cl)c3C)C(=O)NCC4)c2n1] |
| 613 | 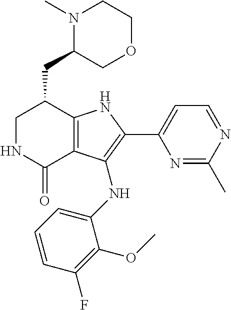 |
| 614 | 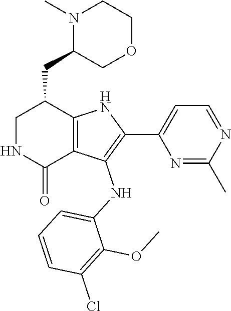 [SMILES: COc1c(Cl)cccc1Nc1c(-c2ccnc(C)n2)[nH]c2c1C(=O)NC[C@@H]2C[C@@H]1COCCN1C] |
| 615 | 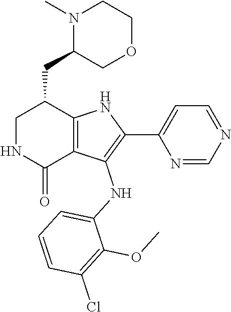 [SMILES: COc1c(Cl)cccc1Nc1c(-c2ccncn2)[nH]c2c1C(=O)NC[C@@H]2C[C@@H]1COCCN1C] |
| 616 | 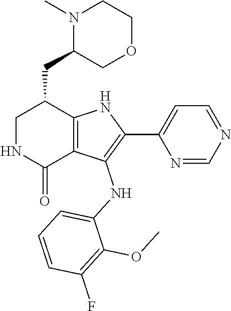 [SMILES: COc1c(F)cccc1Nc1c(-c2ccncn2)[nH]c2c1C(=O)NC[C@@H]2C[C@@H]1COCCN1C] |
| 794 | 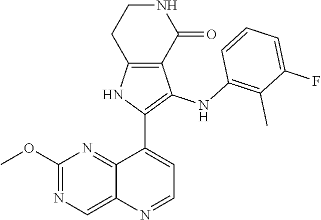 [SMILES: COc1ncc2nccc(-c3[nH]c4c(c3Nc3cccc(F)c3C)C(=O)NCC4)c2n1] |
| 795 | 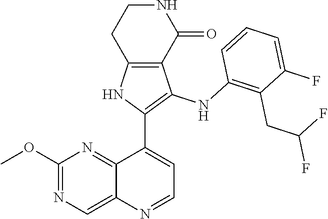 [SMILES: COc1ncc2nccc(-c3[nH]c4c(c3Nc3cccc(F)c3CC(F)F)C(=O)NCC4)c2n1] |
| 796 | 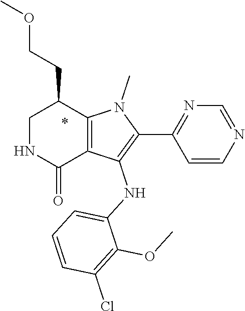 [SMILES: COCC[C@@H]1CNC(=O)c2c(Nc3cccc(Cl)c3OC)c(-c3ccncn3)n(C)c21] |
| 797 | 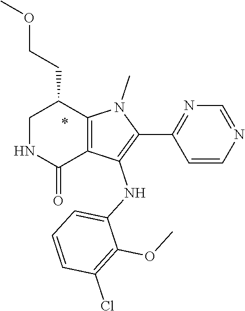 [SMILES: COCC[C@H]1CNC(=O)c2c(Nc3cccc(Cl)c3OC)c(-c3ccncn3)n(C)c21] |
| 617 | 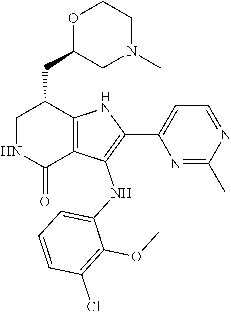 [SMILES: COc1c(Cl)cccc1Nc1c(-c2ccnc(C)n2)[nH]c2c1C(=O)NC[C@@H]2C[C@@H]1CN(C)CCO1] |
| 618 | 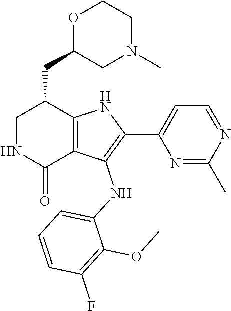 [SMILES: COc1c(F)cccc1Nc1c(-c2ccnc(C)n2)[nH]c2c1C(=O)NC[C@@H]2C[C@@H]1CN(C)CCO1] |
| 619 | 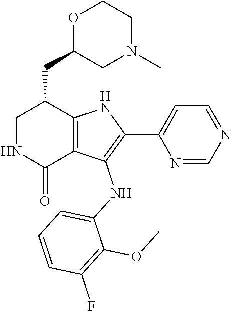 [SMILES: COc1c(F)cccc1Nc1c(-c2ccncn2)[nH]c2c1C(=O)NC[C@@H]2C[C@@H]1CN(C)CCO1] |
| 620 | 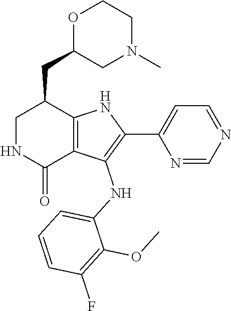 [SMILES: COc1c(F)cccc1Nc1c(-c2ccncn2)[nH]c2c1C(=O)NC[C@H]2C[C@@H]1CN(C)CCO1] |
| 798 | 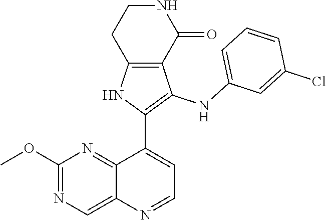 [SMILES: COc1ncc2nccc(-c3[nH]c4c(c3Nc3cccc(Cl)c3)C(=O)NCC4)c2n1] |
| 799 | 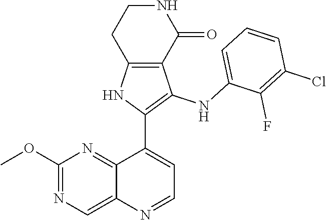 [SMILES: COc1ncc2nccc(-c3[nH]c4c(c3Nc3cccc(Cl)c3F)C(=O)NCC4)c2n1] |
| 800 | 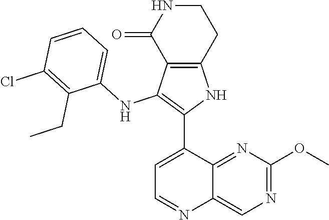 [SMILES: CCc1c(Cl)cccc1Nc1c(-c2ccnc3cnc(OC)nc23)[nH]c2c1C(=O)NCC2] |
| 801 | 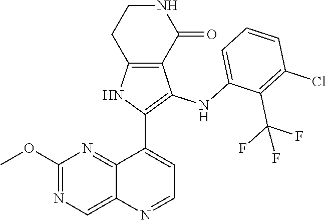 [SMILES: COc1ncc2nccc(-c3[nH]c4c(c3Nc3cccc(Cl)c3C(F)(F)F)C(=O)NCC4)c2n1] |
| 621 | 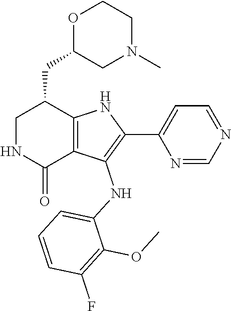 [SMILES: COc1c(F)cccc1Nc1c(-c2ccncn2)[nH]c2c1C(=O)NC[C@@H]2C[C@H]1CN(C)CCO1] |
| 622 | 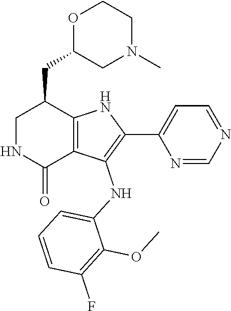 [SMILES: COc1c(F)cccc1Nc1c(-c2ccncn2)[nH]c2c1C(=O)NC[C@H]2C[C@H]1CN(C)CCO1] |
| 623 | 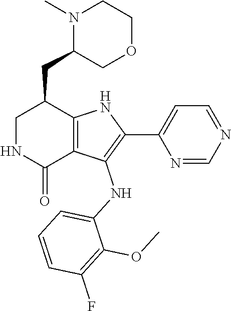 [SMILES: COc1c(F)cccc1Nc1c(-c2ccncn2)[nH]c2c1C(=O)NC[C@H]2C[C@@H]1COCCN1C] |
| 624 | 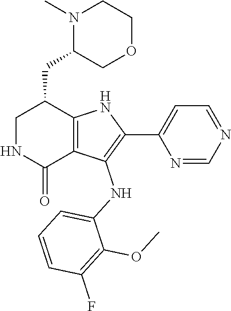 [SMILES: COc1c(F)cccc1Nc1c(-c2ccncn2)[nH]c2c1C(=O)NC[C@@H]2C[C@H]1COCCN1C] |
| 802 | 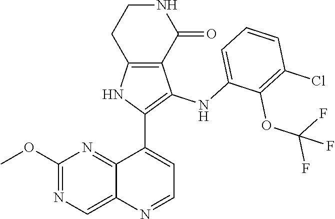 [SMILES: COc1ncc2nccc(-c3[nH]c4c(c3Nc3cccc(Cl)c3OC(F)(F)F)C(=O)NCC4)c2n1] |
| 803 | 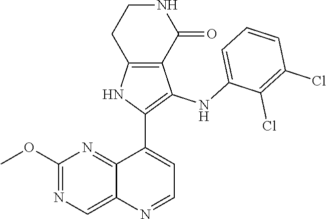 [SMILES: COc1ncc2nccc(-c3[nH]c4c(c3Nc3cccc(Cl)c3Cl)C(=O)NCC4)c2n1] |
| 804 | 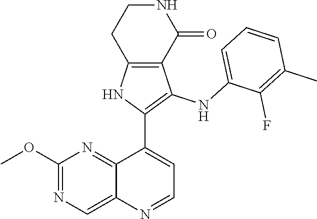 |
| 805 | 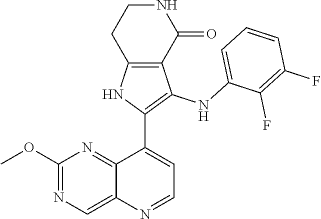 [SMILES: COc1ncc2nccc(-c3[nH]c4c(c3Nc3cccc(F)c3F)C(=O)NCC4)c2n1] |
| 625 | 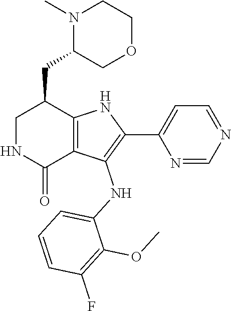 [SMILES: COc1c(F)cccc1Nc1c(-c2ccncn2)[nH]c2c1C(=O)NC[C@H]2C[C@H]1COCCN1C] |
| 626 | 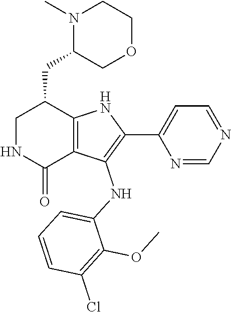 [SMILES: COc1c(Cl)cccc1Nc1c(-c2ccncn2)[nH]c2c1C(=O)NC[C@@H]2C[C@H]1COCCN1C] |
| 627 | 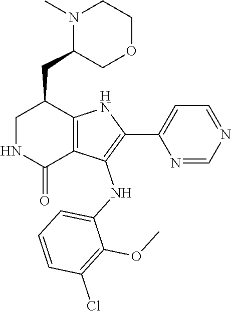 [SMILES: COc1c(Cl)cccc1Nc1c(-c2ccncn2)[nH]c2c1C(=O)NC[C@H]2C[C@@H]1COCCN1C] |
| 628 | 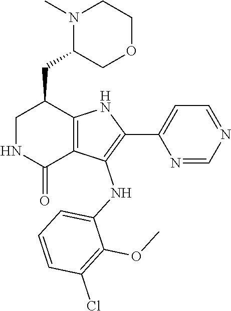 [SMILES: COc1c(Cl)cccc1Nc1c(-c2ccncn2)[nH]c2c1C(=O)NC[C@H]2C[C@H]1COCCN1C] |
| 806 | 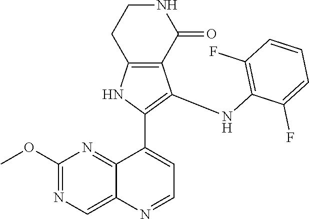 [SMILES: COc1ncc2nccc(-c3[nH]c4c(c3Nc3c(F)cccc3F)C(=O)NCC4)c2n1] |
| 807 | 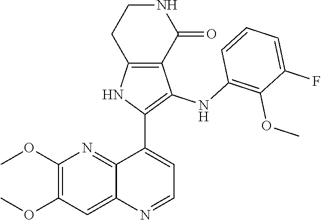 [SMILES: COc1cc2nccc(-c3[nH]c4c(c3Nc3cccc(F)c3OC)C(=O)NCC4)c2nc1OC] |
| 808 | 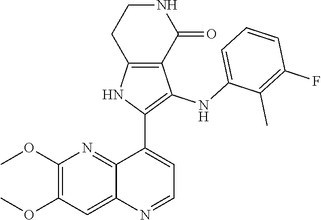 [SMILES: COc1cc2nccc(-c3[nH]c4c(c3Nc3cccc(F)c3C)C(=O)NCC4)c2nc1OC] |
| 809 | 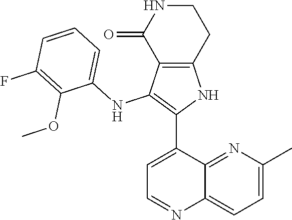 [SMILES: COc1c(F)cccc1Nc1c(-c2ccnc3ccc(C)nc23)[nH]c2c1C(=O)NCC2] |
| 629 | 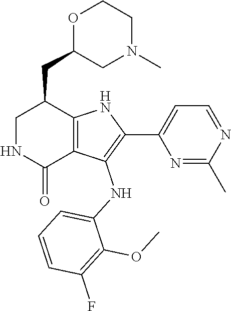 [SMILES: COc1c(F)cccc1Nc1c(-c2ccnc(C)n2)[nH]c2c1C(=O)NC[C@H]2C[C@@H]1CN(C)CCO1] |
| 630 | 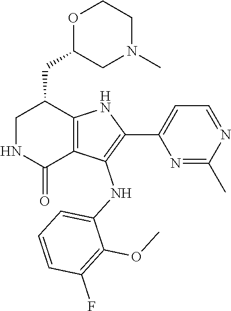 [SMILES: COc1c(F)cccc1Nc1c(-c2ccnc(C)n2)[nH]c2c1C(=O)NC[C@@H]2C[C@H]1CN(C)CCO1] |
| 631 | 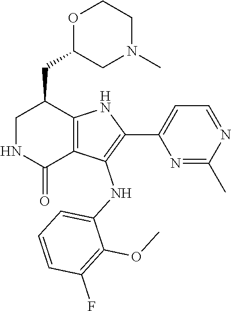 [SMILES: COc1c(F)cccc1Nc1c(-c2ccnc(C)n2)[nH]c2c1C(=O)NC[C@H]2C[C@H]1CN(C)CCO1] |
| 632 | 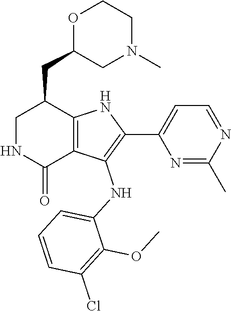 [SMILES: COc1c(Cl)cccc1Nc1c(-c2ccnc(C)n2)[nH]c2c1C(=O)NC[C@H]2C[C@@H]1CN(C)CCO1] |
| 810 | 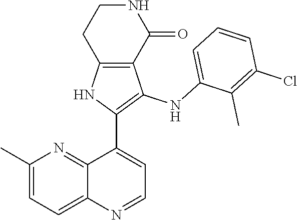 [SMILES: Cc1ccc2nccc(-c3[nH]c4c(c3Nc3cccc(Cl)c3C)C(=O)NCC4)c2n1] |
| 811 | 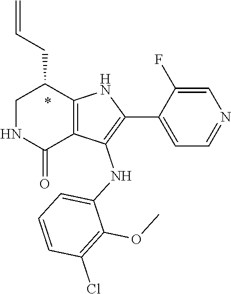 [SMILES: C=CC[C@H]1CNC(=O)c2c1[nH]c(-c1ccncc1F)c2Nc1cccc(Cl)c1OC] |
| 812 | 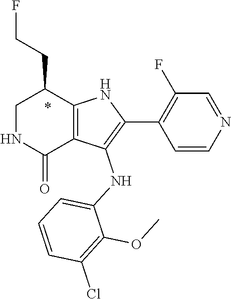 [SMILES: COc1c(Cl)cccc1Nc1c(-c2ccncc2F)[nH]c2c1C(=O)NC[C@H]2CCF] |
| 813 | 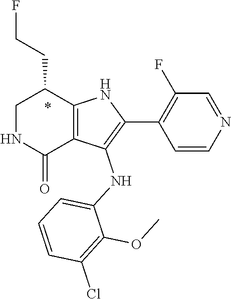 [SMILES: COc1c(Cl)cccc1Nc1c(-c2ccncc2F)[nH]c2c1C(=O)NC[C@@H]2CCF] |
| 633 | 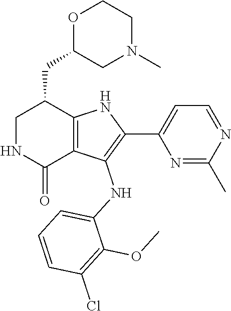 [SMILES: COc1c(Cl)cccc1Nc1c(-c2ccnc(C)n2)[nH]c2c1C(=O)NC[C@@H]2C[C@H]1CN(C)CCO1] |
| 634 | 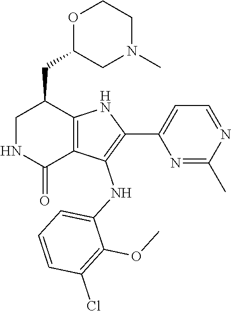 [SMILES: COc1c(Cl)cccc1Nc1c(-c2ccnc(C)n2)[nH]c2c1C(=O)NC[C@H]2C[C@H]1CN(C)CCO1] |
| 635 | 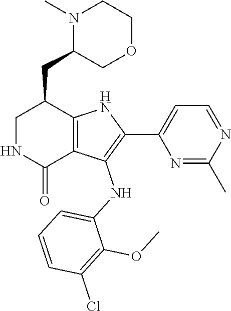 [SMILES: COc1c(Cl)cccc1Nc1c(-c2ccnc(C)n2)[nH]c2c1C(=O)NC[C@H]2C[C@@H]1COCCN1C] |
| 636 | 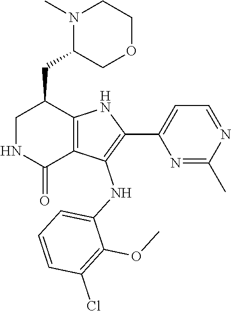 [SMILES: COc1c(Cl)cccc1Nc1c(-c2ccnc(C)n2)[nH]c2c1C(=O)NC[C@H]2C[C@H]1COCCN1C] |
| 814 | 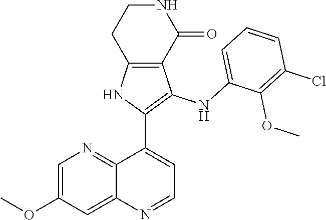 [SMILES: COc1cnc2c(-c3[nH]c4c(c3Nc3cccc(Cl)c3OC)C(=O)NCC4)ccnc2c1] |
| 815 | 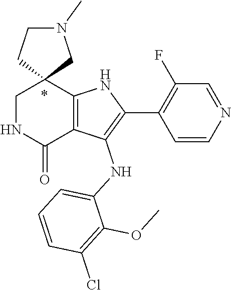 [SMILES: COc1c(Cl)cccc1Nc1c(-c2ccncc2F)[nH]c2c1C(=O)NC[C@]21CCN(C)C1] |
| 816 | 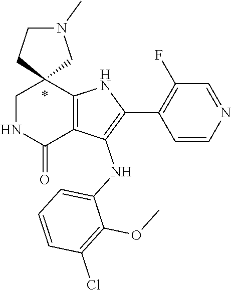 [SMILES: COc1c(Cl)cccc1Nc1c(-c2ccncc2F)[nH]c2c1C(=O)NC[C@@]21CCN(C)C1] |
| 817 | 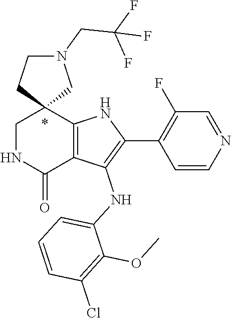 [SMILES: COc1c(Cl)cccc1Nc1c(-c2ccncc2F)[nH]c2c1C(=O)NC[C@@]21CCN(CC(F)(F)F)C1] |
| 637 | 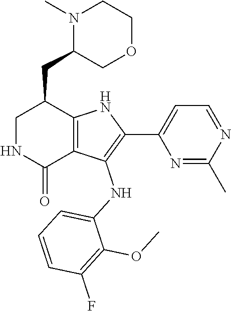 [SMILES: COc1c(F)cccc1Nc1c(-c2ccnc(C)n2)[nH]c2c1C(=O)NC[C@H]2C[C@@H]1COCCN1C] |
| 638 | 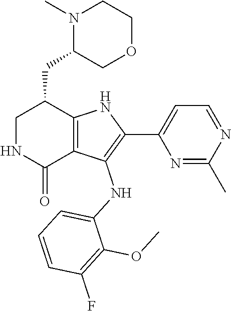 [SMILES: COc1c(F)cccc1Nc1c(-c2ccnc(C)n2)[nH]c2c1C(=O)NC[C@@H]2C[C@H]1COCCN1C] |
| 639 | 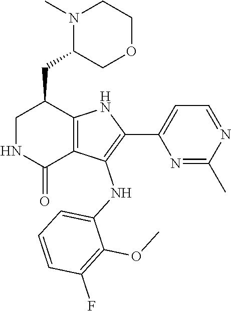 [SMILES: COc1c(F)cccc1Nc1c(-c2ccnc(C)n2)[nH]c2c1C(=O)NC[C@H]2C[C@H]1COCCN1C] |
| 640 | 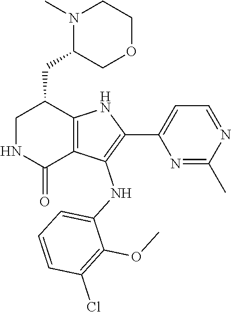 [SMILES: COc1c(Cl)cccc1Nc1c(-c2ccnc(C)n2)[nH]c2c1C(=O)NC[C@@H]2C[C@H]1COCCN1C] |
| 818 | 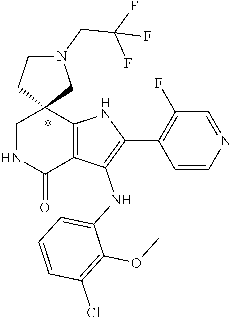 [SMILES: COc1c(Cl)cccc1Nc1c(-c2ccncc2F)[nH]c2c1C(=O)NC[C@]21CCN(CC(F)(F)F)C1] |
| 819 | 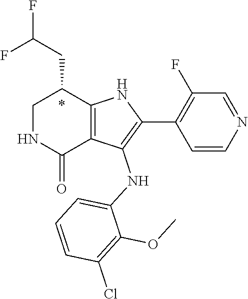 [SMILES: COc1c(Cl)cccc1Nc1c(-c2ccncc2F)[nH]c2c1C(=O)NC[C@@H]2CC(F)F] |
| 820 | 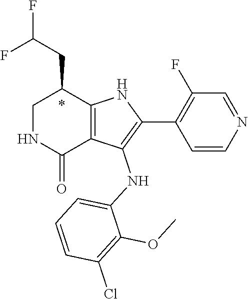 [SMILES: COc1c(Cl)cccc1Nc1c(-c2ccncc2F)[nH]c2c1C(=O)NC[C@H]2CC(F)F] |
| 821 | 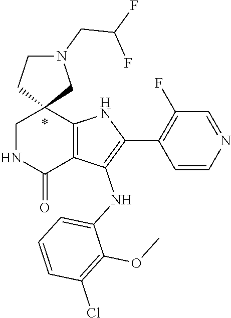 [SMILES: COc1c(Cl)cccc1Nc1c(-c2ccncc2F)[nH]c2c1C(=O)NC[C@]21CCN(CC(F)F)C1] |
| 641 | 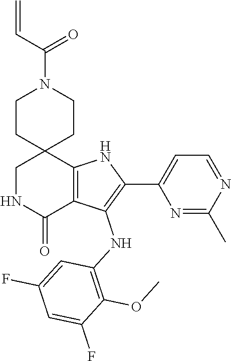 [SMILES: C=CC(=O)N1CCC2(CC1)CNC(=O)c1c2[nH]c(-c2ccnc(C)n2)c1Nc1cc(F)cc(F)c1OC] |
| 642 | 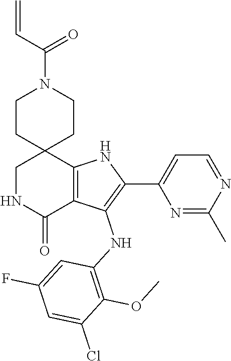 [SMILES: C=CC(=O)N1CCC2(CC1)CNC(=O)c1c2[nH]c(-c2ccnc(C)n2)c1Nc1cc(F)cc(Cl)c1OC] |
| 643 | 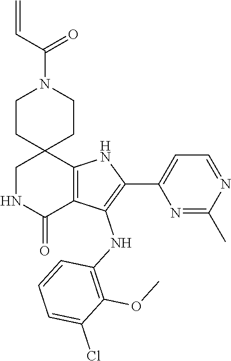 [SMILES: C=CC(=O)N1CCC2(CC1)CNC(=O)c1c2[nH]c(-c2ccnc(C)n2)c1Nc1cccc(Cl)c1OC] |
| 822 | 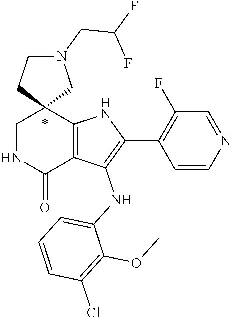 [SMILES: COc1c(Cl)cccc1Nc1c(-c2ccncc2F)[nH]c2c1C(=O)NC[C@@]21CCN(CC(F)F)C1] |
| 823 | 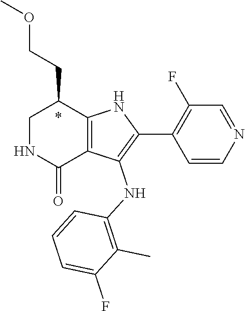 [SMILES: COCC[C@@H]1CNC(=O)c2c1[nH]c(-c1ccncc1F)c2Nc1cccc(F)c1C] |
| 824 | 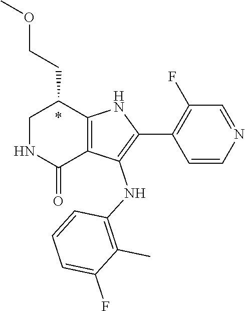 [SMILES: COCC[C@H]1CNC(=O)c2c1[nH]c(-c1ccncc1F)c2Nc1cccc(F)c1C] |
| 644 | 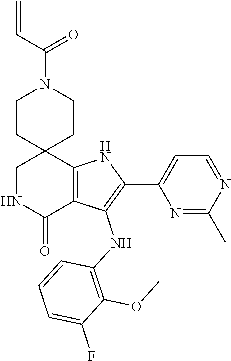 [SMILES: C=CC(=O)N1CCC2(CC1)CNC(=O)c1c2[nH]c(-c2ccnc(C)n2)c1Nc1cccc(F)c1OC] |
| 645 | 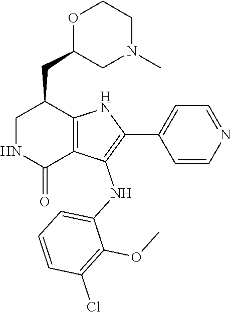 [SMILES: COc1c(Cl)cccc1Nc1c(-c2ccncc2)[nH]c2c1C(=O)NC[C@H]2C[C@@H]1CN(C)CCO1] |
| 646 | 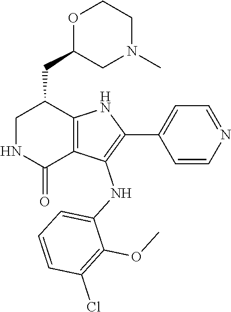 [SMILES: COc1c(Cl)cccc1Nc1c(-c2ccncc2)[nH]c2c1C(=O)NC[C@@H]2C[C@@H]1CN(C)CCO1] |
| 647 | 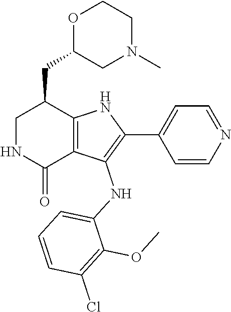 [SMILES: COc1c(Cl)cccc1Nc1c(-c2ccncc2)[nH]c2c1C(=O)NC[C@H]2C[C@H]1CN(C)CCO1] |
| 825 | 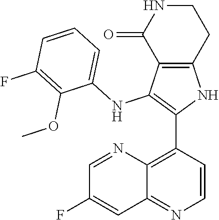 [SMILES: COc1c(F)cccc1Nc1c(-c2ccnc3cc(F)cnc23)[nH]c2c1C(=O)NCC2] |
| 826 | 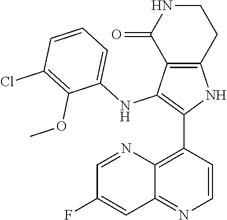 [SMILES: COc1c(Cl)cccc1Nc1c(-c2ccnc3cc(F)cnc23)[nH]c2c1C(=O)NCC2] |
| 827 | 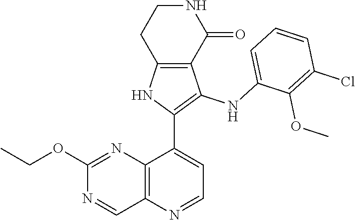 [SMILES: CCOc1ncc2nccc(-c3[nH]c4c(c3Nc3cccc(Cl)c3OC)C(=O)NCC4)c2n1] |
| 828 | 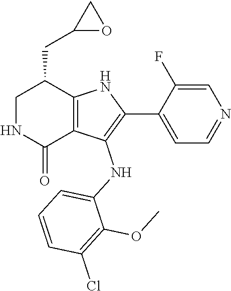 [SMILES: COc1c(Cl)cccc1Nc1c(-c2ccncc2F)[nH]c2c1C(=O)NC[C@@H]2CC1CO1] |
| 648 | 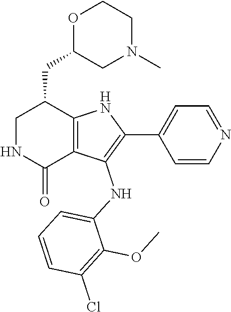 [SMILES: COc1c(Cl)cccc1Nc1c(-c2ccncc2)[nH]c2c1C(=O)NC[C@@H]2C[C@H]1CN(C)CCO1] |
| 649 | 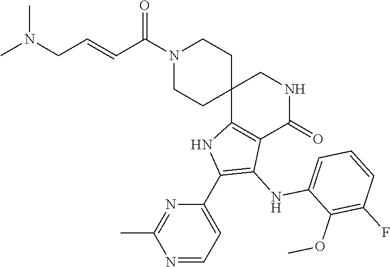 [SMILES: COc1c(F)cccc1Nc1c(-c2ccnc(C)n2)[nH]c2c1C(=O)NCC21CCN(C(=O)/C=C/CN(C)C)CC1] |
| 650 | 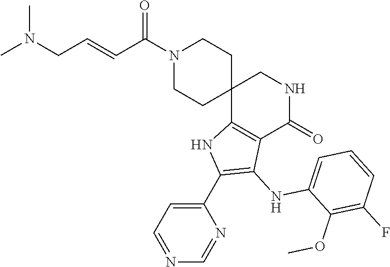 [SMILES: COc1c(F)cccc1Nc1c(-c2ccncn2)[nH]c2c1C(=O)NCC21CCN(C(=O)/C=C/CN(C)C)CC1] |
| 829 | 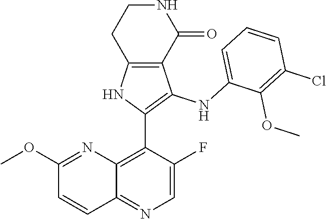 [SMILES: COc1ccc2ncc(F)c(-c3[nH]c4c(c3Nc3cccc(Cl)c3OC)C(=O)NCC4)c2n1] |
| 830 | 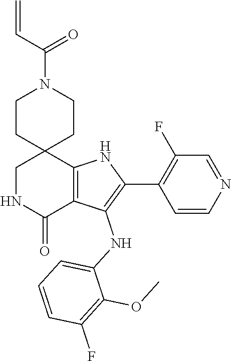 [SMILES: C=CC(=O)N1CCC2(CC1)CNC(=O)c1c2[nH]c(-c2ccncc2F)c1Nc1cccc(F)c1OC] |
| 831 | 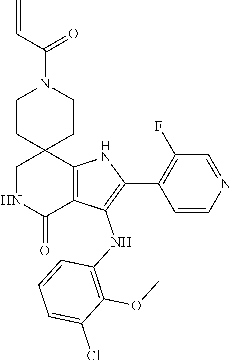 [SMILES: C=CC(=O)N1CCC2(CC1)CNC(=O)c1c2[nH]c(-c2ccncc2F)c1Nc1cccc(Cl)c1OC] |

### Pharmaceutical Compositions and Administration

General

In some embodiments, a chemical entity (e.g., a compound that inhibits EGFR and/or HER2, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like, and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

Indications

Provided herein are methods for inhibiting epidermal growth factor receptor tyrosine kinase (EGFR) and/or human epidermal growth factor receptor 2 (HER2). For example, provided herein are inhibitors of EGFR useful for treating or preventing diseases or disorders associated with dysregulation of an EGFR gene, an EGFR kinase, or the expression or activity or level of any of the same (i.e., an EGFR-associated disease or disorder), such as a central nervous system diseases, a pulmonary disorder, cardiovascular disease, ischemia, liver disease, a gastrointestinal disorder, a viral or bacterial infection, an inflammatory and/or autoimmune disease, or cancer (e.g., EGFR-associated cancer). In some embodiments, provided herein are inhibitors of HER2 useful for treating or preventing diseases or disorders associated with dysregulation of a HER2 gene, a HER2 kinase, or expression or activity or level of any of the same, such as cancer (e.g., HER2-associated cancer). In some embodiments, provided herein are inhibitors of EGFR and HER2.

An “EGFR inhibitor” as used herein includes any compound exhibiting EGFR inactivation activity (e.g., inhibiting or decreasing). In some embodiments, an EGFR inhibitor can be selective for an EGFR kinase having one or more mutations. For example, an EGFR inhibitor can bind to the adenosine triphosphate (ATP)-binding site in the tyrosine kinase domain. In some embodiments, an EGFR inhibitor is an allosteric inhibitor.

The compounds provided herein can inhibit EGFR. In some embodiments, the compounds can bind to the EGFR adenosine triphosphate (ATP)-binding site in the tyrosine kinase domain.

The ability of test compounds to act as inhibitors of EGFR may be demonstrated by assays known in the art. The activity of the compounds and compositions provided herein as EGFR inhibitors can be assayed in vitro, in vivo, or in a cell line. In vitro assays include assays that determine inhibition of the kinase and/or ATPase activity. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radioligands. In some cases, an EGFR inhibitor can be evaluated by its effect on the initial velocity of EGFR tyrosine kinase catalyzed peptide phosphorylation (e.g., Yun et al. Cancer Cell. 2007; 11(3):217-227). In some embodiments, the binding constant of an EGFR inhibitor can be determined using fluorescence kinetics (e.g., Yun et al. Cancer Cell. 2007; 11(3):217-227). Examples of surface plasmon resonance (SPR) binding assays include those disclosed in Li, Shiqing, et al. Cancer cell 7.4 (2005): 301-311. Additional EGFR inhibitor assays can be found, for example, in WO 2019/246541 and WO 2019/165358 both of which are incorporated by reference in their entireties).

Assays can include, for example, proliferation inhibition assays such as those that measure cell growth inhibition, such as an MTS assay or by Cell Titer Glo Luminescent Cell viability assay (Promega®). To perform such an assay, cells are seeded and grown in cell culture plates before being exposed to a test compound for varying durations. Assessment of the viability of the cells following this exposure is then performed. Data are normalized with respect to untreated cells and can be displayed graphically. Growth curves can be fitted using a nonlinear regression model with sigmoidal dose response. As another example, a Western Blot analysis can be used. In such assays cells are seeded and grown in culture plates and then treated with a test compound the following day for varying durations. Cells are washed with PBS and lysed. SDS-PAGE gels are used to separate the lysates which are transferred to nitrocellulose membranes, and probed with appropriate antibodies (e.g., phospho-EGFR(Tyrl 068)(3777), total EGFR (2232), p-Akt(Ser473) (4060), total Akt (9272), p-ERK(Thr202/Tyr204)(4370), total ERK (9102), and HSP90 (SC-7947)).

Additional assays can include, for example, assays based on ALPHALISA TECHNOLOGY® (e.g., see the ALPHALISA® EGF/EGFR binding kit from Promega). Such assays use a luminescent oxygen-channeling chemistry to detect molecules of interest in, for example, buffer, cell culture media, serum, and plasma. For example, a biotinylated EGF is bound to streptavidin-coated Alpha donor beads, and EGFR-Fc is captured by anti-human IgG Fc-specific AlphaLISA acceptor beads. When EGF is bound to EGFR, donor beads and acceptor beads come into close proximity, and the excitation of the donor beads provokes the release of singlet oxygen molecules that triggers a cascade of energy transfers in the acceptor beads. This results in a sharp peak of light emission at 615 nm. Such assays can be used, for example, in competitive binding experiments.

Further examples of assays can include assays based on Sox technology (e.g., see the PHOSPHOSENS® Sox-based Homogeneous, Kinetic or Endpoint/Red Fluorescence-based Assays from ASSAYQUANT®). Such assays utilize chelation-enhanced fluorescence (CHEF) using a sulfonamido-oxine (Sox) chromophore in peptide or protein substrates to create real-time sensors of phosphorylation. See, e.g., U.S. Pat. Nos. 8,586,570 and 6,906,194.

Potency of an EGFR inhibitor as provided herein can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an EGFR-dependent phosphorylation level, in vitro or in vivo (e.g., in tumor cells, A431 cells, Ba/F3 cells, or 3T3 cells expressing a wild type EGFR, a mutant EGFR, or a fragment of any thereof).

Potency of an EGFR inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an EGFR-dependent phosphorylation level, in vitro or in vivo (e.g., in tumor cells, A431 cells, Ba/F3 cells, or 3T3 cells expressing a wild type EGFR, a mutant EGFR, or a fragment of any thereof).

The selectivity between wild type EGFR and EGFR containing one or more mutations as described herein can also be measured using cellular proliferation assays where cell proliferation is dependent on kinase activity. For example, murine Ba/F3 cells transfected with a suitable version of wild type EGFR (such as VIII; containing a wild type EGFR kinase domain), or Ba/F3 cells transfected with L858R/T790M, Del/T790M/L718Q, L858R/T790M/L718Q, L858R/T790M/C797S, Del/T790M/C797S, L858R/T790M/1941R, exon 19 deletion/T790M, or an exon 20 insertion such as V769_D770insX, D770_N771insX, N771_P772insX, P772_H773insX, or H773_V774insX (e.g., A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, or P772_H773insPNP) can be used. Proliferation assays are performed at a range of inhibitor concentrations (e.g., 10 μM, 3 μM, 1.1 μM, 330 nM, 110 nM, 33 nM, 11 nM, 3 nM, 1 nM) and an EC₅₀ is calculated.

An alternative method to measure effects on EGFR activity is to assay EGFR phosphorylation. Wildtype or mutant (L858R/T790M, Del/T790M, Del/T790M/L718Q, L858R/T790M/C797S, Del/T790M/C797S, L858R/T790M/1941R, or L858R/T790M/L718Q) EGFR can be transfected into cells which do not normally express endogenous EGFR and the ability of the inhibitor (e.g., using concentrations as above) to inhibit EGFR phosphorylation can be assayed. Cells are exposed to increasing concentrations of inhibitor and stimulated with EGF. The effects on EGFR phosphorylation are assayed by Western Blotting using phospho-specific EGFR antibodies.

In some embodiments, the compounds provided herein can exhibit potent and selective inhibition of EGFR. For example, the compounds provided herein can bind to the EGFR adenosine triphosphate (ATP)-binding site in the tyrosine kinase domain. In some embodiments, the compounds provided herein can exhibit nanomolar potency against an EGFR kinase including an activating mutation or an EGFR inhibitor resistance mutation, including, for example, the resistance mutations in Table 2a and 2b (e.g., L747S, D761Y, T790M, and T854A), with minimal activity against related kinases (e.g., wild type EGFR). Inhibition of wild type EGFR can cause undesirable side effects (e.g., diarrhea and skin rashes) that can impact quality of life and compliance. In some cases, the inhibition of wild type EGFR can lead to dose limiting toxicities. See, e.g., Morphy. J. Med. Chem. 2010, 53, 4, 1413-1437 and Peters. J. Med. Chem. 2013, 56, 22, 8955-8971.

In some embodiments, the compounds of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target an EGFR kinase. For example, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target an EGFR kinase over another kinase or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit greater inhibition of EGFR containing one or more mutations as described herein (e.g., one or more mutations as described in Table 1a and 1b) relative to inhibition of wild type EGFR. In some embodiments, a compound of Formula (I), or a pharmaceutically acceptable salt thereof can exhibit at least 2-fold, 3-fold, 5-fold, 10-fold, 25-fold, 50-fold or 100-fold greater inhibition of EGFR containing one or more mutations as described herein relative to inhibition of wild type EGFR. In some embodiments, a compound of Formula (I), or a pharmaceutically acceptable salt thereof, can exhibit up to 1000-fold greater inhibition of EGFR containing one or more mutations as described herein relative to inhibition of wild type EGFR. In some embodiments, a compound of Formula (I), or a pharmaceutically acceptable salt thereof, can exhibit up to 10000-fold greater inhibition of EGFR having a combination of mutations described herein relative to inhibition of wild type EGFR.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 2-fold to about 10-fold greater inhibition of EGFR containing one or more mutations as described herein relative to inhibition of wild type EGFR. In some embodiments, a compound of Formula (I), or a pharmaceutically acceptable salt thereof, can exhibit from about 10-fold to about 100-fold greater inhibition of EGFR containing one or more mutations as described herein relative to inhibition of wild type EGFR. In some embodiments, a compound of Formula (I), or a pharmaceutically acceptable salt thereof, can exhibit from about 100-fold to about 1000-fold greater inhibition of EGFR containing one or more mutations as described herein relative to inhibition of wild type EGFR. In some embodiments, a compound of Formula (I), or a pharmaceutically acceptable salt thereof, can exhibit from about 1000-fold to about 10000-fold greater inhibition of EGFR containing one or more mutations as described herein relative to inhibition of wild type EGFR.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit greater inhibition of EGFR containing one or more mutations as described herein (e.g., one or more mutations as described in Table 1a and 1b) relative to inhibition of wild type EGFR. In some embodiments, a compound of Formula (I), or a pharmaceutically acceptable salt thereof, in combination with a second EGFR inhibitor can exhibit at least 2-fold, 3-fold, 5-fold, 10-fold, 25-fold, 50-fold or 100-fold greater inhibition of EGFR containing one or more mutations as described herein relative to inhibition of wild type EGFR. In some embodiments, a compound of Formula (I), or a pharmaceutically acceptable salt thereof, in combination with a second EGFR inhibitor can exhibit up to 1000-fold greater inhibition of EGFR containing one or more mutations as described herein relative to inhibition of wild type EGFR. In some embodiments, a compound of Formula (I), or a pharmaceutically acceptable salt thereof, in combination with a second EGFR inhibitor can exhibit up to 10000-fold greater inhibition of EGFR having a combination of mutations described herein relative to inhibition of wild type EGFR.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit from about 2-fold to about 10-fold greater inhibition of EGFR containing one or more mutations as described herein relative to inhibition of wild type EGFR. In some embodiments, a compound of Formula (I), or a pharmaceutically acceptable salt thereof, in combination with a second EGFR inhibitor can exhibit from about 10-fold to about 100-fold greater inhibition of EGFR containing one or more mutations as described herein relative to inhibition of wild type EGFR. In some embodiments, a compound of Formula (I), or a pharmaceutically acceptable salt thereof, in combination with a second EGFR inhibitor can exhibit from about 100-fold to about 1000-fold greater inhibition of EGFR containing one or more mutations as described herein relative to inhibition of wild type EGFR. In some embodiments, a compound of Formula (I), or a pharmaceutically acceptable salt thereof, in combination with a second EGFR inhibitor can exhibit from about 1000-fold to about 10000-fold greater inhibition of EGFR containing one or more mutations as described herein relative to inhibition of wild type EGFR.

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders which can be treated with an EGFR inhibitor, such as EGFR-associated diseases and disorders, e.g., central nervous system diseases (e.g., neurodegenerative diseases), pulmonary disorders, cardiovascular disease, ischemia, liver disease, gastrointestinal disorders, viral or bacterial infections, inflammatory and/or autoimmune diseases (e.g., psoriasis and atopic dermatitis), and proliferative disorders such as cancers, including hematological cancers and solid tumors (e.g., advanced solid tumors).

A “HER2 inhibitor” as used herein includes any compound exhibiting HER2 inactivation activity (e.g., inhibiting or decreasing). In some embodiments, a HER2 inhibitor can be selective for a HER2 kinase having one or more mutations. In some embodiments, a HER2 inhibitor can bind to the HER2 adenosine triphosphate (ATP)-binding site in the tyrosine kinase domain.

The compounds provided herein can inhibit HER2. For example, the compounds can bind to the HER2 adenosine triphosphate (ATP)-binding site in the tyrosine kinase domain. In some embodiments, the compounds provided herein can inhibit wild type HER2. In some embodiments, the compounds provided herein can inhibit HER2 having one or more mutations as described herein.

The ability of test compounds to act as inhibitors of HER2 may be demonstrated by assays known in the art. The activity of the compounds or compositions provided herein as HER2 inhibitors can be assayed in vitro, in vivo, or in a cell line. In vitro assays include assays that determine inhibition of the kinase and/or ATPase activity. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radioligands. In some cases, a HER2 inhibitor can be evaluated by its effect on the initial velocity of HER2 tyrosine kinase catalyzed peptide phosphorylation (e.g., Yun et al. Cancer Cell. 2007; 11(3):217-227). For example, an assay that indirectly measures ADP formed from the HER2 kinase reaction can be used (see, e.g., ATP/NADH coupled assay systems and luminescent kinase assays such as ADP-GLO™ Kinase Assay from Promega). See, e.g., Hanker et al. Cancer Discov. 2017 June; 7(6):575-585; Robichaux et al. Nat Med. 2018 May; 24(5): 638-646; and Yun et al. Proc Natl Acad Sci USA. 2008 Feb. 12; 105(6):2070-5. In some embodiments, an assay that detects substrate phosphorylation using a labeled anti-phospho-tyrosine antibody can be used (see, e.g., Rabindran et al. Cancer Res. 2004 Jun. 1; 64(11):3958-65). In some embodiments, the binding constant of a HER2 inhibitor can be determined using fluorescence kinetics (e.g., Yun et al. Cancer Cell. 2007; 11(3):217-227). Examples of SPR binding assays include those disclosed in Li, Shiqing, et al. Cancer cell 7.4 (2005): 301-311. In some embodiments, covalent binding of a HER2 inhibitor to HER2 can be detected using mass spectrometry, see, e.g., Irie et al. Mol Cancer Ther. 2019 April; 18(4):733-742. Additional HER2 inhibitor assays can be found, for example, in U.S. Pat. No. 9,920,060, WO 2019/241715, and U.S. Publication No. 2017/0166598, each of which are incorporated by reference in their entireties.

Potency of a HER2 inhibitor as provided herein can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an HER2-dependent phosphorylation level, in vitro or in vivo (e.g., in tumor cells or Ba/F3 cells expressing a wild type HER2, a mutant HER2, or a fragment of any thereof).

Potency of an HER2 inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an HER2-dependent phosphorylation level, in vitro or in vivo (e.g., in tumor cells or Ba/F3 cells expressing a wild type HER2, a mutant HER2, or a fragment of any thereof).

Assays can include, for example, proliferation inhibition assays such as those that measure cell growth inhibition, such as an MTS assay or by Cell Titer Glo Luminescent Cell viability assay (Promega®). To perform such an assay, cells are seeded and grown in cell culture plates before being exposed to a test compound for varying durations. Assessment of the viability of the cells following this exposure is then performed. Data are normalized with respect to untreated cells and can be displayed graphically. Growth curves can be fitted using a nonlinear regression model with sigmoidal dose response. As another example, a Western Blot analysis can be used. In such assays cells are seeded and grown in culture plates and then treated with a test compound the following day for varying durations. Cells are washed with PBS and lysed. SDS-PAGE gels are used to separate the lysates which are transferred to nitrocellulose membranes, and probed with appropriate antibodies (e.g., phospho-HER2(Tyr1248)(2247), phospho-EGFR-Tyr1173 phospho-HER2-Tyr877, phospho-HER2-Tyr1221, total HER2, phospho-AKT-Thr308, phospho-AKT-Ser374, total AKT, phospho-p44/42 MAPK-Thr202/Tyr204, and p44/42 MAPK).

The selectivity between wild type HER2 and HER2 containing one or more mutations as described herein can also be measured using cellular proliferation assays where cell proliferation is dependent on kinase activity. For example, murine Ba/F3 cells transfected with a suitable version of wild type HER2, or Ba/F3 cells transfected with HER2 having one or more mutations such as S310F, S310Y, R678Q, R678W, R678P, I767M, V773M, V777L, V842I, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, or P780_Y781insGSP can be used. Proliferation assays are performed at a range of inhibitor concentrations (e.g., 10 μM, 3 μM, 1.1 μM, 330 nM, 110 nM, 33 nM, 11 nM, 3 nM, 1 nM) and an EC₅₀ is calculated.

An alternative method to measure effects on HER2 activity is to assay HER2 phosphorylation. Wildtype or mutant (S310F, S310Y, R678Q, R678W, R678P, I767M, V773M, V777L, V842I, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, or P780_Y781insGSP) HER2 can be transfected into cells which do not normally express endogenous HER2 and the ability of the inhibitor (e.g., using concentrations as above) to inhibit HER2 phosphorylation can be assayed. Cells are exposed to increasing concentrations of inhibitor and stimulated with EGF. The effects on HER2 phosphorylation are assayed by Western Blotting using phospho-specific HER2 antibodies.

In some embodiments, the compounds provided herein can exhibit potent and selective inhibition of HER2. For example, the compounds provided herein can bind to the HER2 adenosine triphosphate (ATP)-binding site in the tyrosine kinase domain. In some embodiments, the compounds provided herein can exhibit nanomolar potency against a HER2 kinase including an activating mutation or a HER2 inhibitor resistance mutation, including, for example, exon 20 insertions and/or the resistance mutations in Table 5 (e.g., L755S, L755P, T798I, and T798M), with minimal activity against related kinases (e.g., wild type EGFR).

In some embodiments, the compounds of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target a HER2 kinase. For example, a compound of Formula (I), or a pharmaceutically acceptable salt thereof, can selectively target a HER2 kinase over another kinase (e.g., wild type EGFR) or non-kinase target. It can be desirable to selectively target a HER2 kinase over a wild type EGFR kinase due to undesirable side effects (e.g., diarrhea and skin rashes) that can impact quality of life and compliance. See, e.g., Morphy. J. Med. Chem. 2010, 53, 4, 1413-1437 and Peters. J. Med. Chem. 2013, 56, 22, 8955-8971.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit greater inhibition of wild type HER2 or HER2 containing one or more mutations as described herein (e.g., one or more mutations as described in Table 3) relative to inhibition of another kinase (e.g., wild type EGFR) or non-kinase target. In some embodiments, a compound of Formula (I), or a pharmaceutically acceptable salt thereof can exhibit at least 2-fold, 3-fold, 5-fold, 10-fold, 25-fold, 50-fold or 100-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit up to 1000-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit up to 10000-fold greater inhibition of wild type HER2 or HER2 having a combination of mutations described herein relative to inhibition of another kinase (e.g., wild type EGFR) or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 2-fold to about 10-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit from about 10-fold to about 100-fold greater inhibition of wild type HER2 or containing one or more mutations as described herein relative to inhibition of another kinase (e.g., wild type EGFR) or non-kinase target. In some embodiments, a compound of Formula (I), or a pharmaceutically acceptable salt thereof, can exhibit from about 100-fold to about 1000-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit from about 1000-fold to about 10000-fold greater inhibition of wild type HER2 or HER2 containing one or more mutations as described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR inhibitor can exhibit greater inhibition of wild type HER2 or HER2 containing one or more mutations as described herein (e.g., one or more mutations as described in Table 3) relative to inhibition of another kinase (e.g., wild type EGFR) or non-kinase target. In some embodiments, a compound of Formula (I), or a pharmaceutically acceptable salt thereof, in combination with a second HER2 inhibitor can exhibit at least 2-fold, 3-fold, 5-fold, 10-fold, 25-fold, 50-fold or 100-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second HER2 inhibitor can exhibit up to 1000-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second HER2 inhibitor can exhibit up to 10000-fold greater inhibition of wild type HER2 or HER2 having a combination of mutations described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second HER2 inhibitor can exhibit from about 2-fold to about 10-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second HER2 inhibitor can exhibit from about 10-fold to about 100-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second HER2 inhibitor can exhibit from about 100-fold to about 1000-fold greater inhibition of wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second HER2 inhibitor can exhibit from about 1000-fold to about 10000-fold greater inhibition of wild type HER2 or HER2 containing one or more mutations as described herein relative to inhibition of another kinase (e.g., wild type EGFR) or non-kinase target.

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders which can be treated with a HER2 inhibitor, such as HER2-associated diseases and disorders, e.g., proliferative disorders such as cancers (e.g., a HER2-associated cancer), including hematological cancers and solid tumors (e.g., advanced solid tumors).

In some embodiments, the compounds provided herein can also inhibit EGFR and HER2 as described herein.

In some embodiments, the compounds provided herein can exhibit potent and selective inhibition of EGFR and HER2. In some embodiments, the compounds provided herein can exhibit nanomolar potency against an EGFR kinase having one or more mutations, including, for example, one or more of the mutations in Tables 1a, 1b and 2a, 2b, and a HER2 kinase having one or more mutations, including, for example, the mutations in Table 3, with minimal activity against related kinases (e.g., wild type EGFR).

In some embodiments, the compounds of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can selectively target an EGFR and a HER2 kinase. For example, a compound of Formula (I), or a pharmaceutically acceptable salt thereof, can selectively target an EGFR kinase and a HER2 kinase over another kinase or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit greater inhibition of EGFR containing one or more mutations as described herein and wild type HER2 or HER2 containing one or more mutations as described herein (e.g., one or more mutations as described in Tables 3-5) relative to inhibition of another kinase (e.g., wild type EGFR) or non-kinase target. In some embodiments, a compound of Formula (I), or a pharmaceutically acceptable salt thereof can exhibit at least 2-fold, 3-fold, 5-fold, 10-fold, 25-fold, 50-fold or 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit up to 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit up to 10000-fold greater inhibition of EGFR containing one or more mutations as described herein and wild type HER2 or HER2 having one or more mutations described herein relative to inhibition of another kinase (e.g., wild type EGFR) or non-kinase target.

In some embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, can exhibit from about 2-fold to about 10-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit from about 10-fold to about 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit from about 100-fold to about 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, can exhibit from about 1000-fold to about 10000-fold greater inhibition of EGFR containing one or more mutations as described herein and wild type HER2 or HER2 containing one or more mutations as described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit greater inhibition of EGFR containing one or more mutations as described herein and wild type HER2 or HER2 containing one or more mutations as described herein (e.g., one or more mutations as described in Table 3) relative to inhibition of another kinase (e.g., wild type EGFR) or non-kinase target. In some embodiments, a compound of Formula (I), or a pharmaceutically acceptable salt thereof, in combination with a second EGFR and/or second HER2 inhibitor can exhibit at least 2-fold, 3-fold, 5-fold, 10-fold, 25-fold, 50-fold or 100-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second EGFR and/or second HER2 inhibitor can exhibit up to 1000-fold greater inhibition of EGFR containing one or more mutations as described herein and wild type HER2 or HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second EGFR and/or HER2 inhibitor can exhibit up to 10000-fold greater inhibition of EGFR containing one or more mutations as described herein and wild type HER2 or HER2 having a combination of mutations described herein relative to inhibition of another kinase (e.g., wild type EGFR) or non-kinase target.

In other embodiments, a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, in combination with a second EGFR and/or second HER2 inhibitor can exhibit from about 2-fold to about 10-fold greater inhibition of EGFR containing one or more mutations as described herein and HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second EGFR and/or second HER2 inhibitor can exhibit from about 10-fold to about 100-fold greater inhibition of EGFR containing one or more mutations as described herein and HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second EGFR and/or second HER2 inhibitor can exhibit from about 100-fold to about 1000-fold greater inhibition of EGFR containing one or more mutations as described herein and second HER2 containing one or more mutations as described herein relative to inhibition of another kinase (e.g., wild type EGFR) or non-kinase target. In some embodiments, a compound of Formula (I), or a pharmaceutically acceptable salt thereof, in combination with a second EGFR and/or second HER2 inhibitor can exhibit from about 1000-fold to about 10000-fold greater inhibition of EGFR containing one or more mutations as described herein and HER2 containing one or more mutations as described herein relative to inhibition of another kinase (e.g., wild type EGFR) or non-kinase target.

Also provided herein are methods for inhibiting a BUB (budding uninhibited by benzimidazole, BUB1-3) kinase. For example, provided herein are inhibitors of BUB1 kinase useful for treating or preventing diseases or disorders associated with enhanced uncontrolled proliferative cellular processes such as, for example, cancer, inflammation, arthritis, viral diseases, cardiovascular diseases, or fungal diseases. See, for example, WO 2013/050438, WO 2013/092512, WO 2013/167698, WO 2014/147203, WO 2014/147204, WO 2014/202590, WO 2014/202588, WO 2014/202584, WO 2014/202583, WO 2015/063003, WO2015/193339, WO 2016/202755, and WO 2017/021348. In some embodiments, the disease or disorder is cancer.

A “BUB1 inhibitor” as used herein includes any compound exhibiting BUB1 inactivation activity (e.g., inhibiting or decreasing). In some embodiments, a BUB1 inhibitor can be selective for BUB1 over other kinases (e.g., wildtype EGFR).

The compounds provided herein can inhibit a Bub kinase. In some embodiments, the compounds provided herein can inhibit BUB1 kinase.

The ability of test compounds to act as inhibitors of BUB1 may be demonstrated by assays known in the art. The activity of the compounds and compositions provided herein as BUB1 inhibitors can be assayed in vitro, in vivo, or in a cell line. In vitro assays include assays that determine inhibition of the kinase. For example, BUB1 inhibition of a compound provided herein can be determined using a time-resolved fluorescence energy transfer (TR-FRET) assay which measures phosphorylation of a synthetic peptide (e.g., Biotin-AHX-VLLPKKSFAEPG (C-terminus in amide form) by the (recombinant) catalytic domain of human BUB1 (amino acids 704-1085), expressed in Hi5 insect cells with an N-terminal His6-tag and purified by affinity- (Ni-NTA) and size exclusion chromatography. See, for example, WO 2017/021348. In addition, BUB1 activity can be determined at a high ATP concentration using a BUB1 TR-FRET high ATP kinase assay using similar methods as those described above. See, e.g. WO 2019/081486.

In some embodiments, the compounds provided herein exhibit central nervous system (CNS) penetrance. For example, such compounds can be capable of crossing the blood brain barrier (BBB) and inhibiting an EGFR and/or HER2 kinase in the brain and/or other CNS structures. In some embodiments, the compounds provided herein are capable of crossing the blood brain barrier in a therapeutically effective amount. For example, treatment of a patient with cancer (e.g., an EGFR-associated cancer or a HER2-associated cancer such as an EGFR- or HER2-associated brain or CNS cancer or an EGFR-associated or a HER2-associated cancer that has metastasized to the brain or CNS) can include administration (e.g., oral administration) of the compound to the patient.

The ability of the compounds described herein, to cross the BBB can be demonstrated by assays known in the art. Such assays include BBB models such as the transwell system, the hollow fiber (dynamic in vitro BBB) model, other microfluidic BBB systems, the BBB spheroid platform, and other cell aggregate-based BBB models. See, e.g., Cho et al. Nat Commun. 2017; 8: 15623; Bagchi et al. Drug Des Devel Ther. 2019; 13: 3591-3605; Gastfriend et al. Curr Opin Biomed Eng. 2018 March; 5: 6-12; and Wang et al. Biotechnol Bioeng. 2017 January; 114(1): 184-194. In some embodiments, the compounds described herein, are fluorescently labeled, and the fluorescent label can be detected using microscopy (e.g., confocal microscopy). In some such embodiments, the ability of the compound to penetrate the surface barrier of the model can be represented by the fluorescence intensity at a given depth below the surface. In some assays, such as a calcein-AM-based assay, the fluorescent label is non-fluorescent until it permeates live cells and is hydrolyzed by intracellular esterases to produce a fluorescent compound that is retained in the cell and can be quantified with a spectrophotometer. Non-limiting examples of fluorescent labels that can be used in the assays described herein include Cy5, rhodamine, infrared IRDye® CW-800 (LICOR #929-71012), far-red IRDye® 650 (LICOR #929-70020), sodium fluorescein (Na—F), lucifer yellow (LY), 5′carboxyfluorescein, and calcein-acetoxymethylester (calcein-AM). In some embodiments, the BBB model (e.g., the tissue or cell aggregate) can be sectioned, and a compound described herein can be detected in one or more sections using mass spectrometry (e.g., MALDI-MSI analyses). In some embodiments, the ability of a compound described herein to cross the BBB through a transcellular transport system, such as receptor-mediated transport (RMT), carrier-mediated transport (CMT), or active efflux transport (AET), can be demonstrated by assays known in the art. See, e.g., Wang et al. Drug Deliv. 2019; 26(1): 551-565. In some embodiments, assays to determine if compounds can be effluxed by the P-glycoprotein (Pgp) include monolayer efflux assays in which movement of compounds through Pgp is quantified by measuring movement of digoxin, a model Pgp substrate (see, e.g., Doan et al. 2002. J Pharmacol Exp Ther. 303(3):1029-1037). Alternative in vivo assays to identify compounds that pass through the blood-brain barriers include phage-based systems (see, e.g., Peng et al. 2019. ChemRxiv. Preprint doi.org/10.26434/chemrxiv.8242871.v1). In some embodiments, binding of the compounds described herein to brain tissue is quantified. For example, a brain tissue binding assay can be performed using equilibrium dialysis, and the fraction of a compound described herein unbound to brain tissue can be detected using LC-MS/MS (Cyprotex: Brain Tissue Binding Assay www.cyprotex.com/admepk/protein_binding/brain-tissue-binding/).

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders which can be treated with an EGFR inhibitor, a HER2 inhibitor, a dual EGFR and HER2 inhibitor, and/or a BUB1 inhibitor, such as those described herein, e.g., cancer. Accordingly, provided herein is a method for treating a disease or disorder as provided herein in a subject in need thereof, the method comprising administering to the subject a therapeutically effective amount of a compound of Formula (I), or a pharmaceutically acceptable salt or solvate thereof. In some embodiments, the disease or disorder is cancer.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject has been identified or diagnosed as having a cancer with a dysregulation of an EGFR gene, an EGFR protein, or expression or activity, or level of any of the same (an EGFR-associated cancer) (e.g., as determined using a regulatory agency-approved, e.g., FDA-approved, assay or kit). In some embodiments, the subject has a tumor that is positive for a dysregulation of an EGFR gene, an EGFR protein, or expression or activity, or level of any of the same (e.g., as determined using a regulatory agency-approved assay or kit). For example, the subject has a tumor that is positive for a mutation as described in Table 1a and 1b. The subject can be a subject with a tumor(s) that is positive for a dysregulation of an EGFR gene, an EGFR protein, or expression or activity, or level of any of the same (e.g., identified as positive using a regulatory agency-approved, e.g., FDA-approved, assay or kit). The subject can be a subject whose tumors have a dysregulation of an EGFR gene, an EGFR protein, or expression or activity, or a level of the same (e.g., where the tumor is identified as such using a regulatory agency-approved, e.g., FDA-approved, kit or assay). In some embodiments, the subject is suspected of having an EGFR-associated cancer. In some embodiments, the subject has a clinical record indicating that the subject has a tumor that has a dysregulation of an EGFR gene, an EGFR protein, or expression or activity, or level of any of the same (and optionally the clinical record indicates that the subject should be treated with any of the compositions provided herein).

In some embodiments, the subject has been identified or diagnosed as having a cancer with a dysregulation of a HER2 gene, a HER2 protein, or expression or activity, or level of any of the same (a HER2-associated cancer) (e.g., as determined using a regulatory agency-approved, e.g., FDA-approved, assay or kit). In some embodiments, the subject has a tumor that is positive for a dysregulation of a HER2 gene, a HER2 protein, or expression or activity, or level of any of the same (e.g., as determined using a regulatory agency-approved assay or kit). For example, the subject has a tumor that is positive for a mutation as described in Table 3. The subject can be a subject with a tumor(s) that is positive for a dysregulation of a HER2 gene, a HER2 protein, or expression or activity, or level of any of the same (e.g., identified as positive using a regulatory agency-approved, e.g., FDA-approved, assay or kit). The subject can be a subject whose tumors have a dysregulation of a HER2 gene, a HER2 protein, or expression or activity, or a level of the same (e.g., where the tumor is identified as such using a regulatory agency-approved, e.g., FDA-approved, kit or assay). In some embodiments, the subject is suspected of having a HER2-associated cancer. In some embodiments, the subject has a clinical record indicating that the subject has a tumor that has a dysregulation of a HER2 gene, a HER2 protein, or expression or activity, or level of any of the same (and optionally the clinical record indicates that the subject should be treated with any of the compositions provided herein).

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W. B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for preventing diseases and disorders as defined herein (for example, autoimmune diseases, inflammatory diseases, pulmonary disorders, cardiovascular disease, ischemia, liver disease, gastrointestinal disorders, viral or bacterial infections, central nervous system diseases (e.g., neurodegenerative diseases), and cancer). The term “preventing” as used herein means to delay the onset, recurrence or spread, in whole or in part, of the disease or condition as described herein, or a symptom thereof.

The term “EGFR-associated disease or disorder” as used herein refers to diseases or disorders associated with or having a dysregulation of an EGFR gene, an EGFR kinase (also called herein an EGFR kinase protein), or the expression or activity or level of any (e.g., one or more) of the same (e.g., any of the types of dysregulation of an EGFR gene, an EGFR kinase, an EGFR kinase domain, or the expression or activity or level of any of the same described herein). Non-limiting examples of an EGFR-associated disease or disorder include, for example, cancer, a central nervous system disease, a pulmonary disorder, cardiovascular disease, ischemia, liver disease, a gastrointestinal disorder, a viral or bacterial infection, and an inflammatory and/or autoimmune disease (e.g., psoriasis, eczema, atopic dermatitis, and atherosclerosis).

In some embodiments of any of the methods or uses described herein, the inflammatory and/or autoimmune disease is selected from arthritis, systemic lupus erythematosus, atherosclerosis, and skin related disorders such as psoriasis, eczema, and atopic dermatitis. See, e.g., Wang et al. Am J Transl Res. 2019; 11(2): 520-528; Starosyla et al. World J Pharmacol. Dec. 9, 2014; 3(4): 162-173; Choi et al. Biomed Res Int. 2018 May 15; 2018:9439182; and Wang et al. Sci Rep. 2017; 7: 45917.

In some embodiments of any of the methods or uses described herein, the central nervous system disease is a neurodegenerative disease. In some embodiments, the central nervous system disease is selected from Alzheimer's disease, Parkinson's disease, Huntington's disease, amyotrophic lateral sclerosis, spinal cord injury, peripheral neuropathy, brain ischemia, and a psychiatric disorder such as schizophrenia. See, e.g., Iwakura and Nawa. Front Cell Neurosci. 2013 Feb. 13; 7:4; and Chen et al. Sci Rep. 2019 Feb. 21; 9(1):2516.

The term “EGFR-associated cancer” as used herein refers to cancers associated with or having a dysregulation of an EGFR gene, an EGFR kinase (also called herein an EGFR kinase protein), or expression or activity, or level of any of the same. Non-limiting examples of an EGFR-associated cancer are described herein.

The phrase “dysregulation of an EGFR gene, an EGFR kinase, or the expression or activity or level of any of the same” refers to a genetic mutation (e.g., a mutation in an EGFR gene that results in the expression of an EGFR protein that includes a deletion of at least one amino acid as compared to a wild type EGFR protein, a mutation in an EGFR gene that results in the expression of an EGFR protein with one or more point mutations as compared to a wild type EGFR protein, a mutation in an EGFR gene that results in the expression of an EGFR protein with at least one inserted amino acid as compared to a wild type EGFR protein, a gene duplication that results in an increased level of EGFR protein in a cell, or a mutation in a regulatory sequence (e.g., a promoter and/or enhancer) that results in an increased level of EGFR protein in a cell), an alternative spliced version of an EGFR mRNA that results in an EGFR protein having a deletion of at least one amino acid in the EGFR protein as compared to the wild type EGFR protein), or increased expression (e.g., increased levels) of a wild type EGFR kinase in a mammalian cell due to aberrant cell signaling and/or dysregulated autocrine/paracrine signaling (e.g., as compared to a control non-cancerous cell). As another example, a dysregulation of an EGFR gene, an EGFR protein, or expression or activity, or level of any of the same, can be a mutation in an EGFR gene that encodes an EGFR protein that is constitutively active or has increased activity as compared to a protein encoded by an EGFR gene that does not include the mutation. Non-limiting examples of EGFR kinase protein point mutations/insertions/deletions are described in Table 1a and 1b. Additional examples of EGFR kinase protein mutations (e.g., point mutations) are EGFR inhibitor resistance mutations (e.g., EGFR inhibitor mutations). Non-limiting examples of EGFR inhibitor resistance mutations are described in Table 2a and 2b. For example, the one or more EGFR inhibitor resistance mutations can include a substitution at amino acid position 718, 747, 761, 790, 797, or 854 (e.g., L718Q, L747S, D761Y, T790M, C797S, or T854A). Such mutation and overexpression is associated with the development of a variety of cancers (Shan et al., Cell 2012, 149(4) 860-870).

In some embodiments, dysregulation of an EGFR gene, an EGFR kinase, or the expression or activity or level of any of the same can be caused by an activating mutation in an EGFR gene. In some embodiments, dysregulation of an EGFR gene, an EGFR kinase, or the expression or activity or level of any of the same can be caused by a genetic mutation that results in the expression of an EGFR kinase that has increased resistance to an EGFR inhibitor, a tyrosine kinase inhibitor (TKI), and/or a multi-kinase inhibitor (MKI), e.g., as compared to a wild type EGFR kinase (see, e.g., the amino acid substitutions in Table 2a and 2b). In some embodiments, dysregulation of an EGFR gene, an EGFR kinase, or the expression or activity or level of any of the same can be caused by a mutation in a nucleic acid encoding an altered EGFR protein (e.g., an EGFR protein having a mutation (e.g., a primary mutation)) that results in the expression of an altered EGFR protein that has increased resistance to inhibition by an EGFR inhibitor, a tyrosine kinase inhibitor (TKI), and/or a multi-kinase inhibitor (MKI), e.g., as compared to a wild type EGFR kinase (see, e.g., the amino acid substitutions in Table 2a and 2b). The exemplary EGFR kinase point mutations, insertions, and deletions shown in Tables 1a, 1b and 2a, 2b can be caused by an activating mutation and/or can result in the expression of an EGFR kinase that has increased resistance to an EGFR inhibitor), tyrosine kinase inhibitor (TKI), and/or a multi-kinase inhibitor (MKI).

In some embodiments, the individual has two or more EGFR inhibitor resistance mutations that increase resistance of the cancer to a first EGFR inhibitor. For example, the individual can have two EGFR inhibitor resistance mutations. In some embodiments, the two mutations occur in the same EGFR protein. In some embodiments, the two mutations occur in separate EGFR proteins. In some embodiments, the individual can have three EGFR inhibitor resistance mutations. In some embodiments, the three mutations occur in the same EGFR protein. In some embodiments, the three mutations occur in separate EGFR proteins. For example, the individual has two or more EGFR inhibitor resistance mutations selected from Del 19/L718Q, Del 19/T790M, Del 19/L844V, Del 19/T790M/L718Q, Del/T790M/C797S, Del 19/T790M/L844V, L858R/L718Q, L858R/L844V, L858R/T790M, L858R/T790M/L718Q, L858R/T790M/C797S, and L858R/T790M/1941R, or any combination thereof; e.g., any two of the aforementioned EGFR inhibitor resistance mutations.

The term “activating mutation” in reference to EGFR describes a mutation in an EGFR gene that results in the expression of an EGFR kinase that has an increased kinase activity, e.g., as compared to a wild type EGFR kinase, e.g., when assayed under identical conditions. For example, an activating mutation can be a mutation in an EGFR gene that results in the expression of an EGFR kinase that has one or more (e.g., two, three, four, five, six, seven, eight, nine, or ten) amino acid substitutions (e.g., any combination of any of the amino acid substitutions described herein) that has increased kinase activity, e.g., as compared to a wild type EGFR kinase, e.g., when assayed under identical conditions. In another example, an activating mutation can be a mutation in an EGFR gene that results in the expression of an EGFR kinase that has one or more (e.g., two, three, four, five, six, seven, eight, nine, or ten) amino acids deleted, e.g., as compared to a wild type EGFR kinase, e.g., when assayed under identical conditions. In another example, an activating mutation can be a mutation in an EGFR gene that results in the expression of an EGFR kinase that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type EGFR kinase, e.g., the exemplary wild type EGFR kinase described herein, e.g., when assayed under identical conditions. Additional examples of activating mutations are known in the art.

The term “wild type” or “wild-type” describes a nucleic acid (e.g., an EGFR gene or an EGFR mRNA) or protein (e.g., an EGFR protein) sequence that is typically found in a subject that does not have a disease or disorder related to the reference nucleic acid or protein.

The term “wild type EGFR” or “wild-type EGFR” describes an EGFR nucleic acid (e.g., an EGFR gene or an EGFR mRNA) or protein (e.g., an EGFR protein) that is found in a subject that does not have an EGFR-associated disease, e.g., an EGFR-associated cancer (and optionally also does not have an increased risk of developing an EGFR-associated disease and/or is not suspected of having an EGFR-associated disease), or is found in a cell or tissue from a subject that does not have an EGFR-associated disease, e.g., an EGFR-associated cancer (and optionally also does not have an increased risk of developing an EGFR-associated disease and/or is not suspected of having an EGFR-associated disease).

Provided herein is a method of treating cancer (e.g., an EGFR-associated cancer) in a subject in need of such treatment, the method comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. For example, provided herein are methods for treating an EGFR-associated cancer in a subject in need of such treatment, the method comprising a) detecting a dysregulation of an EGFR gene, an EGFR kinase, or the expression or activity or level of any of the same in a sample from the subject; and b) administering a therapeutically effective amount of a compound of Formula (I), or a pharmaceutically acceptable salt thereof. In some embodiments, the dysregulation of an EGFR gene, an EGFR kinase, or the expression or activity or level of any of the same includes one or more EGFR kinase protein point mutations/insertions. Non-limiting examples of EGFR kinase protein point mutations/insertions/deletions are described in Table 1a and 1b. In some embodiments, the EGFR kinase protein point mutations/insertions/deletions are selected from the group consisting of G719S, G719C, G719A, L747S, D761Y, T790M, T854A, L858R, L861Q, a deletion in exon 19 (e.g., L747_A750del), and an insertion in exon 20 (e.g., V769_D770insX, D770_N771insX, N771_P772insX, P772_H773insX, or H773_V774insX). In some embodiments, the EGFR kinase protein point mutations/insertions/deletions are selected from the group consisting of L858R, deletions in exon 19 (e.g., L747_A750del), L747S, D761Y, T790M, and T854A. In some embodiments, the EGFR kinase protein insertion is an exon 20 insertion. In some embodiments, the EGFR kinase protein insertion is an exon 20 insertion selected from the group consisting of: V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP; or any combination thereof; e.g., any two or more independently selected exon 20 insertions; e.g., any two independently selected exon 20 insertions (e.g., V769_D770insASV and D770_N771insSVD).

In some embodiments of any of the methods or uses described herein, the cancer (e.g., EGFR-associated cancer) is selected from a hematological cancer (e.g., acute lymphocytic cancer, Hodgkin lymphoma, non-Hodgkin lymphoma, and leukemia such as acute-myelogenous leukemia (AMVL), chronic-myelogenous leukemia (CML), acute-promyelocytic leukemia, and acute lymphocytic leukemia (ALL)), central or peripheral nervous system tissue cancer, an endocrine or neuroendocrine cancer including multiple neuroendocrine type I and type II tumors, Li-Fraumeni tumors, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, oral cancer, oropharyngeal cancer, nasopharyngeal cancer, respiratory cancer, urogenital cancer, cancer of the vulva, colon cancer, esophageal cancer, tracheal cancer, cervical cancer, gastrointestinal carcinoid tumor, hypopharynx cancer, kidney cancer, larynx cancer, liver cancer, lung cancer, malignant mesothelioma, melanoma, multiple myeloma, nasopharynx cancer, ovarian cancer, pancreatic cancer including pancreatic islet cell cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, parathyroid cancer, pituitary tumors, adrenal gland tumors, ureter cancer, biliary cancer, and urinary bladder cancer. In some embodiments, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In some embodiments, the cancer is pancreatic cancer, head and neck cancer, melanoma, colon cancer, renal cancer, leukemia, lung cancer, or breast cancer. In some cases, the cancer is melanoma, colon cancer, renal cancer, leukemia, or breast cancer.

In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, Liu et al. J Exp Clin Cancer Res. 2019 May 23; 38(1):219); and Ding et al. Cancer Res. 2003 Mar. 1; 63(5):1106-13). In some embodiments, the brain tumor is a primary brain tumor. In some embodiments, the brain tumor is a metastatic brain tumor, e.g., a metastatic brain tumor from lung cancer, melanoma, breast cancer, ovarian cancer, colorectal cancer, kidney cancer, bladder cancer, or undifferentiated carcinoma. In some embodiments, the brain tumor is a metastatic brain tumor from lung cancer (e.g., non-small cell lung cancer). In some embodiments, the compounds provided herein exhibit brain and/or central nervous system (CNS) penetrance. In some embodiments, the patient has previously been treated with another anticancer agent, e.g., another EGFR and/or HER2 inhibitor (e.g., a compound that is not a compound of Formula I) or a multi-kinase inhibitor.

In some embodiments, the cancer is a cancer of B cell origin. In some embodiments, the cancer is a lineage dependent cancer. In some embodiments, the cancer is a lineage dependent cancer where EGFR or the dysregulation of an EGFR gene, an EGFR kinase, or expression or activity or level of any of the same, plays a role in the initiation and/or development of the cancer.

In some embodiments, the cancer is an EGFR-associated cancer. Accordingly, also provided herein is a method for treating a subject diagnosed with or identified as having an EGFR-associated cancer, e.g., any of the exemplary EGFR-associated cancers disclosed herein, comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof as defined herein.

In some embodiments, the dysregulation of an EGFR gene, an EGFR kinase, or expression or activity or level of any of the same, includes one or more deletions (e.g., deletion of an amino acid at position 4), insertions, or point mutation(s) in an EGFR kinase. In some embodiments, dysregulation of an EGFR gene, an EGFR kinase, or expression or activity or level of any of the same, includes at least one deletion, insertion, or point mutation in an EGFR gene that results in the production of an EGFR kinase that has one or more of the amino acid substitutions, insertions, or deletions in Table 1a and 1b. In some embodiments, the dysregulation of an EGFR gene, an EGFR kinase, or expression or activity or level of any of the same, includes a deletion of one or more residues from the EGFR kinase, resulting in constitutive activity of the EGFR kinase domain.

In some embodiments, the dysregulation of an EGFR gene, an EGFR kinase, or expression or activity or level of any of the same, includes at least one point mutation in an EGFR gene that results in the production of an EGFR kinase that has one or more amino acid substitutions, insertions, or deletions as compared to the wild type EGFR kinase (see, for example, the point mutations listed in Table 1a and 1b). In some embodiments, dysregulation of an EGFR gene, an EGFR kinase, or expression or activity or level of any of the same, includes at least one point mutation in an EGFR gene that results in the production of an EGFR kinase that has one or more of the amino acid substitutions, insertions, or deletions in Table 1a and 1b.

In some embodiments, the dysregulation of an EGFR gene, an EGFR kinase, or expression or activity or level of any of the same, includes an insertion of one or more residues in exon 20 of the EGFR gene (e.g., any of the exon 20 insertions described in Table 1a and 1b). Exon 20 of EGFR has two major regions, the c-helix (residues 762-766) and the loop following the c-helix (residues 767-774). Studies suggest that for some exon 20 insertions (e.g., insertions after residue 764), a stabilized and ridged active conformation induces resistance to first generation EGFR inhibitors. In some embodiments, the dysregulation of an EGFR gene, an EGFR kinase, or expression or activity or level of any of the same, includes an insertion of one or more residues in exon 20 selected from the group consisting of: V769_D770insX, D770_N771insX, N771_P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP H773insPNP, or any combination thereof; e.g., any two 10 or more independently selected exon 20 insertions; e.g., any two independently selected exon 20 insertions (e.g., V769_D770insASV and D770_N771insSVD).

TABLE 1a
EGFR Protein Amino Acid Substitutions/Insertions/Deletions^{A}

| Amino Acid | | Non-limiting Exemplary |
|---|---|---|
| Position(s) | Non-Limiting Exemplary Mutations | EGFR-associated Cancer(s) |
| 62 | L62R^{11,B} | |
| 108 | R108K^{11,B} | |
| 216 | A216T^{11,B} | |
| 222 | R222C^{11,B} | |
| 252 | R252C^{11,B} | |
| 289 | A289D, A289T, A289V^{11,B} | |
| 292 | V292L^{11,B} | |
| 304 | H304Y^{11,B} | |
| 306 | S306L^{11,B} | |
| 492 | S492R^{11,B} | |
| 596 | P596L^{11,B} | |
| 598 | GS98V^{11,B} | |
| 688 | L688F⁶ | Lung adenocarcinoma⁶ |
| 689 | V689L⁶ | Lung adenocarcinoma⁶ |
| 703 | L703I, L703P^{11,B} | |
| 706 | I706T⁶ | Lung adenocarcinoma⁶ |
| 709 | E709A, E709G, E709K, E709H, | Lung adenocarcinoma^{2,6}, |
| | E709V^{2,6,9} | NSCLC⁹ |
| 709-710 | E709_T710delinsD⁹ | NSCLC⁹ |
| (sometimes | | |
| also called | | |
| exon 18 | | |
| deletion or | | |
| del_18) | | |
| 714 | K714R^{11,B} | |
| 718 | L718Q^{1,11,B} | |
| 719 | G719S, G719C, G719A, G719D^{1,4} | NSCLC³ |
| 719 and 706 | G719A/I706T⁶ | Lung adenocarcinoma⁶ |
| 719 and 709 | G719S/E709K⁶, G719S/E709A^{11,B}, | Lung adenocarcinoma⁶ |
| | G719C/E709A^{11,B} | |
| 719 and exon | G719X/del_18⁹ | NSCLC⁹ |
| 18 deletion | | |
| 720 | S720F^{11,B} | |
| 735 | G735S^{11,B} | |
| 741 | P741L^{11,B} | |
| 744 | I744M^{11,B} | |
| 747 | L747S^{11,B} | |
| 749 | E749Q⁶ | Lung adenocarcinoma⁶ |
| 750 | A750P^{5,10} | NSCLC⁵, lung |
| | | adenocarcinoma¹⁰ |
| 750 and 749 | A750P/E749Q⁶ | |
| 751 | T751I^{11,B} | |
| 752 | S752F¹⁰ | Lung adenocarcinoma¹⁰ |
| 753 | P753S^{11,B} | |
| 765 | V765A³, V765M^{11,B} | NSCLC³ |
| 767 | A767V^{11,B} | |
| 768 | S768I^{2,9} | Lung adenocarcinoma², |
| | | NSCLC⁹ |
| 768 and 719 | S768I/G719A^{11,B}, S7681/G719C^{11,B}, | |
| | S768I/G7198^{11,B}, | |
| 769 | V769M³, V769L^{11,B} | |
| 771 | N771F⁶ | Lung adenocarcinoma⁶ |
| 773 | H773Y⁶, H773L^{11,B}, H773Y^{11,B} | Lung adenocarcinoma⁶ |
| 774 | V774M, V774A^{2,3} | Lung adenocarcinoma², |
| | | NSCLC³ |
| 774 and 773 | V774M/H773L¹⁰ | NSCLC¹⁰ |
| 776 | R776H, R776C⁶, R776G^{11,B} | Lung adenocarcinoma⁶ |
| 776 and 719 | R776H/G719S⁶ | Lung adenocarcinoma⁶ |
| 779 | G779S^{11,B} | |
| 783 | T783A³ | NSCLC³ |
| 784 | S784P³ | |
| 785 | T785I⁶ | Lung adenocarcinoma⁶ |
| 786 | V786M^{11,B} | |
| 790 | T790M³ | NSCLC³ |
| 790 and 719 | T790M/G719A^{11,B} | |
| 790 and 948 | T790M/V948R¹ | |
| 797 and 790 | C797S/T790M^{11,B} | |
| 798 | P798H^{11,B} | |
| 802 | V802I^{11,B} | |
| 813 | Y813H⁶ | Lung adenocarcinoma⁶ |
| 824 | G824S⁶ | Lung adenocarcinoma⁶ |
| 824 and 688 | G824S/L688F⁶ | Lung adenocarcinoma⁶ |
| 831 | R831H, R831L^{11,B} | |
| 833 | L833V^{2,8}, L833F^{11,B} | Lung adenocarcinoma², |
| | | NSCLC⁸ |
| 834 | V834L, V834M^{11,B} | |
| 835 | H835L^{11,B} | |
| 835, 833, and | H835L/L833V/R670W⁸ | NSCLC⁸ |
| 670 | | |
| 838 | L838V^{11,B} | |
| 843 | V843I³ | NSCLC³ |
| 844 | L844V¹ | |
| 845 | V845M⁶ | Lung adenocarcinoma⁶ |
| 848 | P848L^{11,B} | |
| 851 | V851I⁶ | Lung adenocarcinoma⁶ |
| 854 | T854A^{11,B} | |
| 857 | G857R⁶ | Lung adenocarcinoma⁶ |
| 857, 851, 845, | G857R/V851I/V845M/Y813H/T785I⁶ | Lung adenocarcinoma⁶ |
| 813, and 785 | | |
| 858 | L858R¹ | |
| 858 and 108 | L858R/R108K^{11,B} | |
| 858 and 289 | L858R/A289T^{11,B} | |
| 858 and 292 | L858R/V292L^{11,B} | |
| 858 and 306 | L858R/S306L^{11,B} | |
| 858 and 703 | L858R/L703I^{11,B} | |
| 858 and 709 | L858R/E709A^{11,B}, L858R/E709G^{11,B}, | |
| | L858R/E709K^{11,B}, L858R/E709V^{11,B}, | |
| 858 and 714 | L858R/K714R^{11,B} | |
| 858 and 718 | L858R/L718Q¹ | |
| 858 and 720 | L858R/S720F^{11,B} | |
| 858 and 744 | L858R/I744M^{11,B} | |
| 858 and 768 | L858R/S768I^{11,B} | |
| 858 and 769 | L858R/V769L⁶ | Lung adenocarcinoma⁶ |
| 858 and 776 | L858R/R776H⁶, L858R/R776C^{11,B}, | Lung adenocarcinoma⁶ |
| | L858R/R776G^{11,B} | |
| 858 and 790 | L858R/T790M^{11,B} | |
| 858 and 833 | L858R/L833V⁶ | Lung adenocarcinoma⁶ |
| 858 and 838 | L858R/L838V^{11,B} | |
| 858 and 843 | L858R/V843I^{11,B} | |
| 858 and 844 | L858R/L844V¹ | |
| 858 and exon | L858R/del_18⁹ | NSCLC⁹ |
| 18 deletion | | |
| 859 | A859T^{11,B} | |
| 860 | K860R^{11,B} | |
| 861 | L861Q, L861R, L681G^{1,5,6,7,9} | Lung adenocarcinoma⁶, |
| | | NSCLC^{5,9} |
| 861 and 719 | L861Q/G719X¹⁰, L861Q/G719A^{11,B} | Lung adenocarcinoma¹⁰ |
| | L861R/G719A^{11,B} | |
| 861 and 858 | L861Q/L858R^{11,B} | |
| 861, 768, and | L861Q/S7681/G719X¹⁰ | Lung adenocarcinoma¹⁰ |
| 719 | | |
| 864 | A864T^{11,B} | |
| 865 | E865K^{11,B} | |
| 870 | H870R⁶ | Lung adenocarcinoma⁶ |
| 870 and 858 | H870R/L858R | |
| 871 | A871E, A871G^{3,6}, A871T^{11,B} | Lung adenocarcinoma⁶ |
| 871 and 858 | A871G/L858R⁶ | Lung adenocarcinoma⁶ |
| 873 | G873E^{11,B} | |
| 874 | G874S^{11,B} | |
| 941 | I941R¹ | |
| 948 | V948R¹ | |
| 1118 | A1118T^{11,B} | |
| 1153 | S1153I^{11,B} | |
| Exon 19 insertion (sometimes also called ins_19) | | |
| | V738_K739insKIPVAI⁶ | Lung adenocarcinoma⁶ |
| | I744_K745insKIPVAI^{9,10} | NSCLC^{9,10} |
| | K745_E746insTPVAIK^{9,10} | NSCLC^{9,10} |
| | K745_E746insVPVAIK¹⁰ | NSCLC¹⁰ |
| | K745_E746insIPVAIK^{9,10} | NSCLC^{9,10} |
| Exon 19 deletion (sometimes also called del_19)^{1,2} | | |
| | E746_A750del¹ | |
| | E746_A750delinsP¹ | |
| | E746_A750delinsIP^{11,B} | |
| | E746_A750del/T790M^{11,B} | |
| | E746_A750del/A1118T^{11,B} | |
| | E746_T751delinsV^{11,B} | |
| | E746_S752delinsV² | Lung adenocarcinoma² |
| | E746_S752delinsV/A216T^{11,B} | |
| | L747_E749del⁵ | NSCLC⁵ |
| | L747_A750del³ | NSCLC³ |
| | L747_A750delinsP² | Lung adenocarcinoma² |
| | L747_T751del² | Lung adenocarcinoma² |
| | L747_T751delinsA¹⁰ | NSCLC¹⁰ |
| | L747_T751delinsP¹⁰ | NSCLC¹⁰ |
| | L747_T751delinsS^{11,B} | |
| | L747_T751delinsQ² | Lung adenocarcinoma² |
| | L747_S752del² | Lung adenocarcinoma² |
| | L747_P753del¹⁰ | NSCLC¹⁰ |
| | L747_P753delinsS² | Lung adenocarcinoma² |
| | L747_P753delinsQ^{11,B} | |
| | L747_P753delinsVS¹⁰ | NSCLC¹⁰ |
| | T751_I759delinsN^{11,B} | |
| | S752_I759del^{2,10} | Lung adenocarcinoma², |
| | | NSCLC¹⁰ |
| Exon 19 | Del_19 and I706T⁶ | Lung adenocarcinoma⁶ |
| deletion and | | |
| 706 | | |
| Exon 19 | Del_19 and L718Q¹ | |
| deletion and | | |
| 718 | | |
| Exon 19 | Del_19 and L844V¹ | |
| deletion and | | |
| 844 | | |
| Exon 19 | Del_19 and L858R⁶ | Lung adenocarcinoma⁶ |
| deletion and | | |
| 858 | | |
| Exon 19 | Del_19 and del_18⁹ | NSCLC⁹ |
| deletion and | | |
| Exon 18 | | |
| deletion | | |
| Exon 20 insertion (sometimes also called ins_20)¹ | | |
| | D761_E762insX⁵, e.g., | NSCLC⁵ |
| | D761_E762insEAFQ¹⁰ | |
| | A763_Y764insX⁵, e.g., | NSCLC^{5,9,10} |
| | A763_Y764insFQEA^{5,9,10} | |
| | Y764_V765insX⁵, e.g., | NSCLC⁵ |
| | V764_V765insHH¹³ | |
| | V765_M766insX⁵ | NSCLC⁵ |
| | M766_A767insASV⁶ | Lung adenocarcinoma⁶ |
| | A767_S768insX⁵; A767insASV¹⁴ | NSCLC⁵ |
| | A767_V769dupASV⁵ | NSCLC⁵ |
| | S768_V769insX¹²; S768dupSVD¹⁴ | NSCLC |
| | V769_D770insX⁵, e.g., | NSCLC^{5,8,10} |
| | V769_D770insASV^{8,9,10} | |
| | D770delinsGY^{5,10} | NSCLC^{5,10} |
| | D770_N771insX⁵, e.g., | NSCLC^{3,5,10} |
| | D770_N771insNPG^{2,5,9}, | |
| | D770_N771insG + N771T⁶, | |
| | D770_N771insNPY⁶, | |
| | D770_N771insSVD^{9,10}, | |
| | D770_N771insGL¹³ | |
| | D770_N771insX and amino acid position | Lung adenocarcinoma⁶ |
| | 773, e.g., D770_N771insNPY/H773Y⁶ | |
| | N771_P772insX⁵, e.g., N771_P772insN¹⁰, | NSCLC^{5,10} |
| | N771_P772insH¹³, N771_P772insV¹³ | |
| | N771_H773dupNPH⁵ | NSCLC⁵ |
| | N771delinsGY¹³; N771del insFH¹⁴ | |
| | P772_H773insX⁵, e.g., | NSCLC^{5,9} |
| | P772_H773insDNP⁹, P772_H773insPNP⁹ | |
| | H773_V774insX⁵, e.g., | NSCLC^{5,9,10} |
| | H773_V774insNPH⁹, H773_V774insH¹⁰, | |
| | H773_V774insPH¹⁰, H773_V774insAH¹⁰, | |
| | P772_H773insPNP¹³ | |
| | H773_dupH¹³ | |
| | V774_C775insX⁵, e.g., | NSCLC⁵ |
| | V774_C775insHV¹⁰ | |
| | A775_G776insX, e.g., | |
| | A775_G776insYVMA¹³ | |
| Kinase domain | Duplication of exons 18-25, 18-26, 14-26, | NSCLC⁹ |
| duplication | or 17-25⁹ | |
| (KDD) | | |

^{A}The EGFR mutations shown may be activating mutations and/or confer increased resistance of EGFR to an EGFR inhibitor and/or a multi-kinase inhibitor (MKI), e.g., as compared to a wild type EGFR
^{B}Potentially oncogenic variant. See, e.g., Kohsaka, Shinji, et al. Science translational medicine 9.416 (2017): eaan6566.
¹PCT Patent Application Publication No. WO2019/246541.
²Grosse A, Grosse C, Rechsteiner M, Soltermann A. Diagn Pathol. 2019; 14(1): 18. Published 2019 Feb, 11. doi: 10.1186/s13000-019-0789-1.
³Stewart E L, Tan S Z, Liu G, Tsao M S. Transl Lung Cancer Res. 2015; 4(1): 67-81. doi: 10.3978/j.issn.2218-6751.2014.11.06.
⁴Pines, Gur, Wolfgang J. Köstler, and Yosef Yarden. FEBS letters 584.12 (2010): 2699-2706.
⁵Yasuda, Hiroyuki, Susumu Kobayashi, and Daniel B. Costa. The Lancet Oncology 13.1 (2012): e23-e31.
⁶Kim E Y, Cho E N, Park H S, et al. Cancer Biol Ther. 2016; 17(3): 237-245. doi: 10.1080/15384047.2016.1139235.
⁷Shah, Riyaz, and Jason F. Lester. Clinical Lung Cancer (2019).
⁸Aran, Veronica, and Jasminka Omerovic. International journal of molecular sciences 20.22 (2019): 5701. doi: 10.3390/ijms20225701.
⁹Beau-Faller, Michele, et al. (2012): 10507-10507. doi: 10.1016/j.semcancer.2019.09.015.
¹⁰Masood, Ashiq, Rama Krishna Kancha, and Janakiraman Subramanian. Seminars in oncology. W B Saunders, 2019. doi: 10.1053/j.seminoncol.2019.08.004.
¹¹Kohsaka, Shinji, et al. Science translational medicine 9.416 (2017): eaan6566.
¹²Vyse and Huang et al. Signal Transduct Target Ther. 2019 Mar, 8; 4: 5. doi: 10.1038/s41392-019-0038-9.
¹³PCT Patent Application Publication No. WO2019/046775.
¹⁴PCT Patent Application Publication No. WO 2018/094225.

TABLE 1b
EGFR Protein Amino Acid Substitutions/Insertions/Deletions^{A}

| Amino Acid | | Non-limiting Exemplary |
|---|---|---|
| Position(s) | Non-Limiting Exemplary Mutations | EGFR-associated Cancer(s) |
| 62 | L62R^{11,B} | |
| 108 | R108K^{11,B} | |
| 216 | A216T^{11,B} | |
| 222 | R222C^{11,B} | |
| 252 | R252C^{11,B} | |
| 289 | A289D, A289T, A289V^{11,B} | |
| 292 | V292L^{11,B} | |
| 304 | H304Y^{11,B} | |
| 306 | S306L^{11,B} | |
| 492 | S492R^{11,B} | |
| 596 | P596L^{11,B} | |
| 598 | GS98V^{11,B} | |
| 688 | L688F⁶ | Lung adenocarcinoma⁶ |
| 689 | V689L⁶ | Lung adenocarcinoma⁶ |
| 703 | L703I, L703P^{11,B} | |
| 706 | I706T⁶ | Lung adenocarcinoma⁶ |
| 709 | E709A, E709G, E709K, E709H, | Lung adenocarcinoma^{2,6}, |
| | E709V^{2,6,9} | NSCLC⁹ |
| 709-710 | E709_T710delinsD⁹ | NSCLC⁹ |
| (sometimes | | |
| also called | | |
| exon 18 | | |
| deletion or | | |
| del_18) | | |
| 714 | K714R^{11,B} | |
| 718 | L718Q^{1,11,B} | |
| 719 | G719S, G719C, G719A, G719D^{1,4} | NSCLC³ |
| 719 and 706 | G719A/I706T⁶ | Lung adenocarcinoma⁶ |
| 719 and 709 | G719S/E709K⁶, G719S/E709A^{11,B}, | Lung adenocarcinoma⁶ |
| | G719C/E709A^{11,B} | |
| 719 and exon | G719X/del_18⁹ | NSCLC⁹ |
| 18 deletion | | |
| 720 | S720F^{11,B} | |
| 735 | G735S^{11,B} | |
| 741 | P741L^{11,B} | |
| 744 | I744M^{11,B} | |
| 747 | L747S^{11,B} | |
| 749 | E749Q⁶ | Lung adenocarcinoma⁶ |
| 750 | A750P^{5,10} | NSCLC⁵, lung |
| | | adenocarcinoma¹⁰ |
| 750 and 749 | A750P/E749Q⁶ | |
| 751 | T751I^{11,B} | |
| 752 | S752F¹⁰ | Lung adenocarcinoma¹⁰ |
| 753 | P753S^{11,B} | |
| 765 | V765A³, V765M^{11,B} | NSCLC³ |
| 767 | A767V^{11,B} | |
| 768 | S768I^{2,9} | Lung adenocarcinoma², |
| | | NSCLC⁹ |
| 768 and 719 | S768I/G719A^{11,B}, S7681/G719C^{11,B}, | |
| | S768I/G7198^{11,B}, | |
| 769 | V769M³, V769L^{11,B} | |
| 771 | N771F⁶ | Lung adenocarcinoma⁶ |
| 773 | H773Y⁶, H773L^{11,B}, H773Y^{11,B} | Lung adenocarcinoma⁶ |
| 774 | V774M, V774A^{2,3} | Lung adenocarcinoma², |
| | | NSCLC³ |
| 774 and 773 | V774M/H773L¹⁰ | NSCLC¹⁰ |
| 776 | R776H, R776C⁶, R776G^{11,B} | Lung adenocarcinoma⁶ |
| 776 and 719 | R776H/G719S⁶ | Lung adenocarcinoma⁶ |
| 779 | G779S^{11,B} | |
| 783 | T783A³ | NSCLC³ |
| 784 | S784P³ | |
| 785 | T785I⁶ | Lung adenocarcinoma⁶ |
| 786 | V786M^{11,B} | |
| 790 | T790M³ | NSCLC³ |
| 790 and 719 | T790M/G719A^{11,B} | |
| 790 and 948 | T790M/V948R¹ | |
| 797 and 790 | C797S/T790M^{11,B} | |
| 798 | P798H^{11,B} | |
| 802 | V802I^{11,B} | |
| 813 | Y813H⁶ | Lung adenocarcinoma⁶ |
| 824 | G824S⁶ | Lung adenocarcinoma⁶ |
| 824 and 688 | G824S/L688F⁶ | Lung adenocarcinoma⁶ |
| 831 | R831H, R831L^{11,B} | |
| 833 | L833V^{2,8}, L833F^{11,B} | Lung adenocarcinoma², |
| | | NSCLC⁸ |
| 834 | V834L, V834M^{11,B} | |
| 835 | H835L^{11,B} | |
| 835, 833, and | H835L/L833V/R670W⁸ | NSCLC⁸ |
| 670 | | |
| 838 | L838V^{11,B} | |
| 843 | V843I³ | NSCLC³ |
| 844 | L844V¹ | |
| 845 | V845M⁶ | Lung adenocarcinoma⁶ |
| 848 | P848L^{11,B} | |
| 851 | V851I⁶ | Lung adenocarcinoma⁶ |
| 854 | T854A^{11,B} | |
| 857 | G857R⁶ | Lung adenocarcinoma⁶ |
| 857, 851, 845, | G857R/V851I/V845M/Y813H/T785I⁶ | Lung adenocarcinoma⁶ |
| 813, and 785 | | |
| 858 | L858R¹ | |
| 858 and 108 | L858R/R108K^{11,B} | |
| 858 and 289 | L858R/A289T^{11,B} | |
| 858 and 292 | L858R/V292L^{11,B} | |
| 858 and 306 | L858R/S306L^{11,B} | |
| 858 and 703 | L858R/L703I^{11,B} | |
| 858 and 709 | L858R/E709A^{11,B}, L858R/E709G^{11,B}, | |
| | L858R/E709K^{11,B}, L858R/E709V^{11,B}, | |
| 858 and 714 | L858R/K714R^{11,B} | |
| 858 and 718 | L858R/L718Q¹ | |
| 858 and 720 | L858R/S720F^{11,B} | |
| 858 and 744 | L858R/I744M^{11,B} | |
| 858 and 768 | L858R/S768I^{11,B} | |
| 858 and 769 | L858R/V769L⁶ | Lung adenocarcinoma⁶ |
| 858 and 776 | L858R/R776H⁶, L858R/R776C^{11,B}, | Lung adenocarcinoma⁶ |
| | L858R/R776G^{11,B} | |
| 858 and 790 | L858R/T790M^{11,B} | |
| 858 and 833 | L858R/L833V⁶ | Lung adenocarcinoma⁶ |
| 858 and 838 | L858R/L838V^{11,B} | |
| 858 and 843 | L858R/V843I^{11,B} | |
| 858 and 844 | L858R/L844V¹ | |
| 858 and exon | L858R/del_18⁹ | NSCLC⁹ |
| 18 deletion | | |
| 859 | A859T^{11,B} | |
| 860 | K860R^{11,B} | |
| 861 | L861Q, L861R, L681G^{1,5,6,7,9} | Lung adenocarcinoma⁶, |
| | | NSCLC^{5,9} |
| 861 and 719 | L861Q/G719X¹⁰, L861Q/G719A^{11,B}, | Lung adenocarcinoma¹⁰ |
| | L861R/G719A^{11,B} | |
| 861 and 858 | L861Q/L858R^{11,B} | |
| 861, 768, and | L861Q/S7681/G719X¹⁰ | Lung adenocarcinoma¹⁰ |
| 719 | | |
| 864 | A864T^{11,B} | |
| 865 | E865K^{11,B} | |
| 870 | H870R⁶ | Lung adenocarcinoma⁶ |
| 870 and 858 | H870R/L858R | |
| 871 | A871E, A871G^{3,6}, A871T^{11,B} | Lung adenocarcinoma⁶ |
| 871 and 858 | A871G/L858R⁶ | Lung adenocarcinoma⁶ |
| 873 | G873E^{11,B} | |
| 874 | G874S^{11,B} | |
| 941 | I941R¹ | |
| 948 | V948R¹ | |
| 1118 | A1118T^{11,B} | |
| 1153 | S1153I^{11,B} | |
| Exon 19 insertion (sometimes also called ins_19) | | |
| | V738_K739insKIPVAI⁶ | Lung adenocarcinoma⁶ |
| | I744_K745insKIPVAI^{9,10} | NSCLC^{9,10} |
| | K745_E746insTPVAIK^{9,10} | NSCLC^{9,10} |
| | K745_E746insVPVAIK¹⁰ | NSCLC¹⁰ |
| | K745_E746insIPVAIK^{9,10} | NSCLC^{9,10} |
| Exon 19 deletion (sometimes also called del_19)^{1,2} | | |
| | E746_A750del¹ | |
| | E746_A750delinsP¹ | |
| | E746_A750delinsIP^{11,B} | |
| | E746_A750del/T790M^{11,B} | |
| | E746_A750del/A1118T^{11,B} | |
| | E746_T751delinsV^{11,B} | |
| | E746_S752delinsV² | Lung adenocarcinoma² |
| | E746_S752delinsV/A216T^{11,B} | |
| | L747_E749del⁵ | NSCLC⁵ |
| | L747_A750del³ | NSCLC³ |
| | L747_A750delinsP² | Lung adenocarcinoma² |
| | L747_T751del² | Lung adenocarcinoma² |
| | L747_T751delinsA¹⁰ | NSCLC¹⁰ |
| | L747_T751delinsP¹⁰ | NSCLC¹⁰ |
| | L747_T751delinsS^{11,B} | |
| | L747_T751delinsQ² | Lung adenocarcinoma² |
| | L747_S752del² | Lung adenocarcinoma² |
| | L747_P753del¹⁰ | NSCLC¹⁰ |
| | L747_P753delinsS² | Lung adenocarcinoma² |
| | L747_P753delinsQ^{11,B} | |
| | L747_P753delinsVS¹⁰ | NSCLC¹⁰ |
| | T751_I759delinsN^{11,B} | |
| | S752_I759del^{2,10} | Lung adenocarcinoma², |
| | | NSCLC¹⁰ |
| Exon 19 | Del_19 and I706T⁶ | Lung adenocarcinoma⁶ |
| deletion and | | |
| 706 | | |
| Exon 19 | Del_19 and L718Q¹ | |
| deletion and | | |
| 718 | | |
| Exon 19 | Del_19 and L844V¹ | |
| deletion and | | |
| 844 | | |
| Exon 19 | Del_19 and L858R⁶ | Lung adenocarcinoma⁶ |
| deletion and | | |
| 858 | | |
| Exon 19 | Del_19 and del_18⁹ | NSCLC⁹ |
| deletion and | | |
| Exon 18 | | |
| deletion | | |
| Exon 20 insertion (sometimes also called ins_20)¹ | | |
| | D761_E762insX⁵, e.g., | NSCLC⁵ |
| | D761_E762insEAFQ¹⁰ | |
| | A763_Y764insX⁵, e.g., | NSCLC^{5,9,10} |
| | A763_Y764insFQEA^{5,9,10} | |
| | Y764_V765insX⁵, e.g., | NSCLC⁵ |
| | V764_V765insHH¹³ | |
| | V765_M766insX⁵ | NSCLC⁵ |
| | M766_A767insASV⁶ | Lung adenocarcinoma⁶ |
| | M766delinsMASVx2¹⁵ | Pediatric bithalamic |
| | | glioma¹⁵ |
| | A767_S768insX⁵; A767insASV¹⁴ | NSCLC⁵ |
| | A767_V769dupASV⁵ | NSCLC⁵ |
| | A767delinsASVDx3; | Pediatric bithalamic |
| | A767delinsASVG¹⁵ | glioma¹⁵ |
| | S768_V769insX¹²; S768dupSVD^{14,16} | NSCLC^{12,14}; Sinonasal |
| | | squamous cell |
| | | carcinoma¹⁶ |
| | V769_D770insX⁵, e.g., | NSCLC^{5,8,10} |
| | V769_D770insASV^{8,9,10} | |
| | D770delinsGY^{5,10}; D770delinsDN; | NSCLC^{5,10}; Pediatric |
| | D770delinsDNPH¹⁵ | bithalamic glioma¹⁵ |
| | D770_N771insX^{5,16}, e.g., | NSCLC^{3,5,10}; Sinonasal |
| | D770_N771insNPG^{2,5,9}, | squamous cell |
| | D770_N771insG + N771T⁶, | carcinoma¹⁶ |
| | D770_N771insNPY⁶, | |
| | D770_N771insSVD^{9,10}, | |
| | D770_N771insGL¹³ | |
| | D770_N771insX and amino acid position | Lung adenocarcinoma⁶ |
| | 773, e.g., D770_N771insNPY/H773Y⁶ | |
| | N771_P772insX^{5,16}, e.g., N771_P772insN¹⁰, | NSCLC^{5,10} Sinonasal |
| | N771_P772insH¹³, N771_P772insV¹³ | squamous cell |
| | | carcinoma¹⁶ |
| | N771_H773dupNPH⁵ | NSCLC⁵ |
| | N771delinsGY¹³; N771del insFH¹⁴; | NSCLC^{13,14}; Pediatric |
| | N771delinsNPH¹⁵ | bithalamic glioma¹⁵ |
| | N771_H773dup¹⁶ | Sinonasal squamous |
| | | cell carcinoma¹⁶ |
| | P772_H773insX⁵, e.g., | NSCLC^{5,9} |
| | P772_H773insDNP⁹, P772_H773insPNP⁹ | |
| | H773_V774insX⁵, e.g., | NSCLC^{5,9,10} |
| | H773_V774insNPH⁹, H773_V774insH¹⁰, | |
| | H773_V774insPH¹⁰, H773_V774insAH¹⁰, | |
| | P772_H773insPNP¹³ | |
| | H773_dupH¹³ | |
| | V774_C775insX⁵, e.g., | NSCLC⁵ |
| | V774_C775insHV¹⁰ | |
| | A775_G776insX, e.g., | |
| | A775_G776insYVMA¹³ | |
| Kinase domain | Duplication of exons 18-25, 18-26, 14-26, | NSCLC⁹ |
| duplication | or 17-25⁹ | |
| (KDD) | | |

^{A}The EGFR mutations shown may be activating mutations and/or confer increased resistance of EGFR to an EGFR inhibitor and/or a multi-kinase inhibitor (MKI), e.g., as compared to a wild type EGFR
^{B}Potentially oncogenic variant. See, e.g., Kohsaka, Shinji, et al. Science translational medicine 9.416 (2017): eaan6566.
¹PCT Patent Application Publication No. WO2019/246541.
²Grosse A, Grosse C, Rechsteiner M, Soltermann A. Diagn Pathol. 2019; 14(1): 18. Published 2019 Feb. 11. doi: 10.1186/s13000-019-0789-1.
³Stewart E L, Tan S Z, Liu G, Tsao M S. Transl Lung Cancer Res. 2015; 4(1): 67-81. doi: 10.3978/j.issn.2218-6751.2014.11.06.
⁴Pines, Gur, Wolfgang J. Köstler, and Yosef Yarden. FEBS letters 584.12 (2010): 2699-2706.
⁵Yasuda, Hiroyuki, Susumu Kobayashi, and Daniel B. Costa. The Lancet Oncology 13.1 (2012): e23-e31.
⁶Kim E Y, Cho E N, Park H S, et al. Cancer Biol Ther. 2016; 17(3): 237-245. doi: 10.1080/15384047.2016.1139235.
⁷Shah, Riyaz, and Jason F. Lester. Clinical Lung Cancer (2019).
⁸Aran, Veronica, and Jasminka Omerovic. International journal of molecular sciences 20.22 (2019): 5701. doi: 10.3390/ijms20225701.
⁹Beau-Faller, Michele, et al. (2012): 10507-10507. doi: 10.1016/j.semcancer.2019.09.015.
¹⁰Masood, Ashiq, Rama Krishna Kancha, and Janakiraman Subramanian. Seminars in oncology. W B Saunders, 2019. doi: 10.1053/j.seminoncol.2019.08.004.
¹¹Kohsaka, Shinji, et al. Science translational medicine 9.416 (2017): eaan6566.
¹²Vyse and Huang et al. Signal Transduct Target Ther. 2019 Mar. 8; 4: 5. doi: 10.1038/s41392-019-0038-9.
¹³PCT Patent Application Publication No. WO2019/046775.
¹⁴PCT Patent Application Publication No. WO 2018/094225.
¹⁵Mondal, Gourish, et al. Acta Neuropathol. 2020; 139(6): 1071-1088
¹⁶Udager, Aaron M., et al. Cancer Res, 2015; 75(13): 2600-2606

In some embodiments, the dysregulation of an EGFR gene, an EGFR kinase, or expression or activity or level of any of the same, includes a splice variation in an EGFR mRNA which results in an expressed protein that is an alternatively spliced variant of EGFR having at least one residue deleted (as compared to the wild type EGFR kinase) resulting in a constitutive activity of an EGFR kinase domain.

In some embodiments, the dysregulation of an EGFR gene, an EGFR kinase, or expression or activity or level of any of the same, includes at least one point mutation in an EGFR gene that results in the production of an EGFR kinase that has one or more amino acid substitutions or insertions or deletions in an EGFR gene that results in the production of an EGFR kinase that has one or more amino acids inserted or removed, as compared to the wild type EGFR kinase. In some cases, the resulting EGFR kinase is more resistant to inhibition (e.g., inhibition of its signaling activity) by one or more first EGFR inhibitors, as compared to a wild type EGFR kinase or an EGFR kinase not including the same mutation. Such mutations, optionally, do not decrease the sensitivity of the cancer cell or tumor having the EGFR kinase to treatment with a compound of Formula (I)(e.g., Formula (I-a), (I-b), (I-c), (I-d), (I-e), (I-f), (I-g), (I-h), (I-i), (I-j), or (I-k)), or a pharmaceutically acceptable salt thereof. (e.g., as compared to a cancer cell or a tumor that does not include the particular EGFR inhibitor resistance mutation).

In other embodiments, the dysregulation of an EGFR gene, an EGFR kinase, or expression or activity or level of any of the same, includes at least one point mutation in an EGFR gene that results in the production of an EGFR kinase that has one or more amino acid substitutions as compared to the wild type EGFR kinase, and which has increased resistance to a compound of Formula (I), or a pharmaceutically acceptable salt thereof, as compared to a wild type EGFR kinase or an EGFR kinase not including the same mutation. In such embodiments, an EGFR inhibitor resistance mutation can result in an EGFR kinase that has one or more of an increased Vₘₐₓ, a decreased Kₘ, and a decreased K_{D} in the presence of a compound of Formula (I), or a pharmaceutically acceptable salt thereof, as compared to a wild type EGFR kinase or an EGFR kinase not having the same mutation in the presence of the same compound of Formula (I), or a pharmaceutically acceptable salt thereof.

Exemplary Sequence of Mature Human EGFR Protein (UniProtKB entry P00533)
(SEQ ID NO: 1)
MRPSGTAGAA LLALLAALCP ASRALEEKKV CQGTSNKLTQ LGTFEDHELS
LQRMENNCEV VIGNLEITYV QRNYDLSFLK TIQEVAGYVL IALNTVERIP
LENLQIIRGN MYYENSYALA VLSNYDANKT GLKELPMRNL QEILHGAVRF
SNNPALCNVE SIQWRDIVSS DELSNMSMDE QNHLGSCQKC DPSCPNGSCW
GAGEENCQKL TKIICAQQCS GRCRGKSPSD CCHNQCAAGC TGPRESDCLV
CRKFRDEATC KDTCPPLMLY NPTTYQMDVN PEGKYSFGAT CVKKCPRNYV
VTDHGSCVRA CGADSYEMEE DGVRKCKKCE GPCRKVCNGI GIGEFKDSLS
INATNIKHFK NCTSISGDLH ILPVAFRGDS FTHTPPLDPQ ELDILKTVKE
ITGFLLIQAW PENRTDLHAF ENLEIIRGRT KQHGQFSLAV VSINITSLGL
RSLKEISDGD VIISGNKNLC YANTINWKKL FGTSGQKTKI ISNRGENSCK
ATGQVCHALC SPEGCWGPEP RDCVSCRNVS RGRECVDKCN LLEGEPREFV
ENSECIQCHP ECLPQAMNIT CTGRGPDNCI QCAHYIDGPH CVKTCPAGVM
GENNTLVWKY ADAGHVCHLC HPNCTYGCTG PGLEGCPTNG PKIPSIATGM
VGALLLLLVV ALGIGLEMRR RHIVRKRTLR RLLQERELVE PLTPSGEAPN
QALLRILKET EFKKIKVLGS GAFGTVYKGL WIPEGEKVKI PVAIKELREA
TSPKANKEIL DEAYVMASVD NPHVCRLLGI CLISTVQLIT QLMPFGCLLD
YVREHKDNIG SQYLLNWCVQ IAKGMNYLED RRLVHRDLAA RNVLVKTPQH
VKITDEGLAK LIGAEEKEYH AEGGKVPIKW MALESILHRI YTHQSDVWSY
GVTVWELMTF GSKPYDGIPA SEISSILEKG ERLPQPPICT IDVYMIMVKC
WMIDADSRPK FRELIIEFSK MARDPQRYLV IQGDERMHLP SPTDSNFYRA
LMDEEDMDDV VDADEYLIPQ QGFFSSPSTS RTPLLSSLSA TSNNSTVACI
DRNGLQSCPI KEDSFLQRYS SDPTGALTED SIDDTFLPVP EYINQSVPKR
PAGSVQNPVY HNQPLNPAPS RDPHYQDPHS TAVGNPEYLN TVQPTCVNST
FDSPAHWAQK GSHQISLDNP DYQQDFFPKE AKPNGIFKGS TAENAEYLRV
APQSSEFIGA

In some embodiments, dysregulation of an EGFR gene, an EGFR kinase, or expression or activity or level of any of the same, includes at least one EGFR inhibitor resistance mutation in an EGFR gene that results in the production of an EGFR kinase that has one or more of the amino acid substitutions, insertions, or deletions as described in Table 2a and 2b. In some embodiments, compounds of Formula (I) (e.g., Formula (I-a), (I-b), (I-c), (I-d), (I-e), (I-f), (I-g), (I-h), (I-i), (I-j), or (I-k)) and pharmaceutically acceptable salts and solvates thereof, are useful in treating subjects that develop cancers with EGFR inhibitor resistance mutations (e.g., that result in an increased resistance to a first EGFR inhibitor, e.g., a substitution at amino acid position 718, 747, 761, 790, 797, or 854 (e.g., L718Q, L747S, D761Y, T790M, C797S, T854A), and/or one or more EGFR inhibitor resistance mutations listed in Table 2a and 2b) by either dosing in combination or as a subsequent or additional (e.g., follow-up) therapy to existing drug treatments (e.g., other inhibitors of EGFR; e.g., first and/or second EGFR inhibitors).

TABLE 2a
EGFR Protein Amino Acid Resistance Mutations

| Amino Acid | | Non-limiting Exemplary |
|---|---|---|
| Position(s) | Non-Limiting Exemplary Mutations | EGFR-associated Cancer(s) |
| 747 | L747S^{2,4,6}, L747P⁶ | NSCLC² |
| 761 | D761Y^{2,4} | NSCLC² |
| 769 | V769M² | NSCLC² |
| 790 | T790M^{1,4} | NSCLC² |
| 792 | L792H⁶ | NSCLC⁶ |
| 796 | G796R⁶ | NSCLC⁶ |
| 797 | C7978⁵ | NSCLC⁵ |
| 797 and 790 | C797S/T790M⁵ | NSCLC⁵ |
| 843 | V843I^{2,5} | NSCLC² |
| 854 | T854A^{2,4} | NSCLC² |
| 858 and 747 | L858R/L747S⁶ | NSCLC⁶ |
| 858 and 790 | L858R/T790M¹ | |
| 858, 797, and | L858R/C797S/T790M¹ | |
| 790 | | |
| 871 | A871E² | NSCLC² |
| 941, 858, and | I941R/L858R/T790M¹ | |
| 790 | | |
| Exon 19 | Del_19 and T790M¹ | |
| deletion and | | |
| 790 | | |
| Exon 19 | Del_19 and T790M/L844V¹ | |
| deletion, 844, | | |
| and 790 | | |
| Exon 19 | Del_19 and C797S/T790M¹ | |
| deletion, 797, | | |
| and 790 | | |
| Exon 20 insertion (also called ins_20)^{2,3} | | |
| | A767_V769dupASV⁵ | NSCLC⁵ |
| | D770_N771insX³, e.g., | NSCLC^{2,3} |
| | D770_N771insNPG^{2,3,7} | |
| | N771_H771dupNPH³ | NSCLC³ |
| | P772_H773insX³ e.g., | NSCLC^{3,7} |
| | P772_H773insDNP⁷ | |
| | H773_V774insNPH⁷ | NSCLC⁷ |

¹PCT Patent Application Publication No. WO2019/246541
²Stewart E L, Tan S Z, Liu G, Tsao M S. Transl Lung Cancer Res. 2015; 4(1): 67-81. doi: 10.3978/j.issn.2218-6751.2014.11.06
³Yasuda, Hiroyuki, Susumu Kobayashi, and Daniel B. Costa. The Lancet Oncology 13.1 (2012): e23-e31.
⁴Kim E Y, Cho E N, Park H S, et al. Cancer Biol Ther. 2016; 17(3): 237-245. doi: 10.1080/15384047.2016.1139235
⁵Shah, Riyaz, and Jason F. Lester. Clinical Lung Cancer (2019).
⁶Aran, Veronica, and Jasminka Omerovic. International journal of molecular sciences 20.22 (2019): 5701. doi: 10.3390/ijms20225701.
⁷Beau-Faller, Michele, et al. (2012): 10507-10507. doi: 10.1016/j.semcancer.2019.09.015
⁸Masood, Ashiq, Rama Krishna Kancha, and Janakiraman Subramanian. Seminars in oncology. W B Saunders, 2019. doi: 10.1053/j.seminoncol.2019.08.004

TABLE 2b
EGFR Protein Amino Acid Resistance Mutations

| Amino Acid | | Non-limiting Exemplary |
|---|---|---|
| Position(s) | Non-Limiting Exemplary Mutations | EGFR-associated Cancer(s) |
| 747 | L747S^{2,4,6}, L747P⁶ | NSCLC² |
| 761 | D761Y^{2,4} | NSCLC² |
| 769 | V769M² | NSCLC² |
| 790 | T790M^{1,4} | NSCLC² |
| 792 | L792H⁶ | NSCLC⁶ |
| 796 | G796R⁶ | NSCLC⁶ |
| 797 | C7978⁵ | NSCLC⁵ |
| 797 and 790 | C797S/T790M⁵ | NSCLC⁵ |
| 843 | V843I^{2,5} | NSCLC² |
| 854 | T854A^{2,4} | NSCLC² |
| 858 and 747 | L858R/L747S⁶ | NSCLC⁶ |
| 858 and 790 | L858R/T790M¹ | |
| 858 and 797 | L858R/C797S (or C797G)⁹ | NSCLC⁹ |
| 858, 797, and | L858R/C797S/T790M¹ | |
| 790 | | |
| 871 | A871E² | NSCLC² |
| 941, 858, and | I941R/L858R/T790M¹ | |
| 790 | | |
| Exon 19 | Del_19 and T790M¹ | |
| deletion and | | |
| 790 | | |
| Exon 19 | Del_19 and C797S (or C797G)⁹ | NSCLC⁹ |
| deletion and | | |
| 797 | | |
| Exon 19 | Del_19 and T790M/L844V¹ | |
| deletion, 844, | | |
| and 790 | | |
| Exon 19 | Del_19 and C797S/T790M¹ | |
| deletion, 797, | | |
| and 790 | | |
| Exon 20 insertion (also called ins_20)^{2,3} | | |
| | A767_V769dupASV⁵ | NSCLC⁵ |
| | D770_N771insX³, e.g., | NSCLC^{2,3} |
| | D770_N771insNPG^{2,3,7} | |
| | N771_H771dupNPH³ | NSCLC³ |
| | P772_H773insX³ e.g., | NSCLC^{3,7} |
| | P772_H773insDNP⁷ | |
| | H773_V774insNPH⁷ | NSCLC⁷ |

¹PCT Patent Application Publication No. WO2019/246541
²Stewart E L, Tan S Z, Liu G, Tsao M S. Transl Lung Cancer Res. 2015; 4(1): 67-81. doi: 10.3978/j.issn.2218-6751.2014.11.06
³Yasuda, Hiroyuki, Susumu Kobayashi, and Daniel B. Costa. The Lancet Oncology 13.1 (2012): e23-e31.
⁴Kim E Y, Cho E N, Park H S, et al. Cancer Biol Ther. 2016; 17(3): 237-245. doi: 10.1080/15384047.2016.1139235
⁵Shah, Riyaz, and Jason F. Lester. Clinical Lung Cancer (2019).
⁶Aran, Veronica, and Jasminka Omerovic. International journal of molecular sciences 20.22 (2019): 5701. doi: 10.3390/ijms20225701.
⁷Beau-Faller, Michele, et al. (2012): 10507-10507. doi: 10.1016/j.semcancer.2019.09.015
⁸Masood, Ashiq, Rama Krishna Kancha, and Janakiraman Subramanian. Seminars in oncology. W B Saunders, 2019. doi: 10.1053/j.seminoncol.2019.08.004
⁹Papadimitrakopoulou, V. A., et al. Annals of Oncology 2018; 29 Supplement 8 VIII741

In some embodiments, the EGFR Protein Amino Acid Substitutions/Insertions/Deletions include any one or more, or any two or more (e.g., any two), of the EGFR Protein Amino Acid Substitutions/Insertions/Deletions delineated in Table 1a, 1b and/or Table 2a, 2b; e.g., any one or more, or any two or more (e.g., any two), of the following and independently selected EGFR Protein Amino Acid Substitutions/Insertions/Deletions: V769L; V769M; M766delinsMASVx2; A767_V769dupASV; A767delinsASVDx3; A767delinsASVG; S768_V769insX; V769_D770insX; V769_D770insASV; D770delinsDN; D770delinsDNPH; D770_N771insSV; N771delinsNPH; N771_H773dup; L858R/C797S (or C797G); or Del_19 and C797S (or C797G), or any combination thereof.

As used herein, a “first inhibitor of EGFR” or “first EGFR inhibitor” is an EGFR inhibitor as defined herein, but which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof as defined herein. As used herein, a “second inhibitor of EGFR” or a “second EGFR inhibitor” is an EGFR inhibitor as defined herein, but which does not include a compound of Formula (I), or a pharmaceutically acceptable salt thereof, as defined herein. When both a first and a second inhibitor of EGFR are present in a method provided herein, the first and second inhibitors of EGFR are different. In some embodiments, the first and/or second inhibitor of EGFR bind in a different location than a compound of Formula (I). For example, in some embodiments, a first and/or second inhibitor of EGFR can inhibit dimerization of EGFR, while a compound of Formula (I) can inhibit the active site. In some embodiments, a first and/or second EGFR inhibitor can be an allosteric inhibitor of EGFR, while a compound of Formula (I) can inhibit the EGFR active site.

Exemplary first and second inhibitors of EGFR are described herein. In some embodiments, a first or second inhibitor of EGFR can be selected from the group consisting of osimertinib, gefitinib, erlotinib, afatinib, lapatinib, neratinib, AZD-9291, CL-387785, CO-1686, or WZ4002.

In some embodiments, compounds of Formula (I) (e.g., Formula (I-a), (I-b), (I-c), (I-d), (I-e), (I-f), (I-g), (I-h), (I-i), (I-j), or (I-k)), or pharmaceutically acceptable salts and solvates thereof, are useful for treating a cancer that has been identified as having one or more EGFR inhibitor resistance mutations (that result in an increased resistance to a first or second inhibitor of EGFR, e.g., a substitution described in Table 2a and 2b including substitutions at amino acid position 747, 761, 790, 797, or 854 (e.g., L718Q, L747S, D761Y, T790M, C797S, T854A)). In some embodiments, the one or more EGFR inhibitor resistance mutations occurs in a nucleic acid sequence encoding a mutant EGFR protein (e.g., a mutant EGFR protein having any of the mutations described in Table 2a and 2b) resulting in a mutant EGFR protein that exhibits EGFR inhibitor resistance.

The epidermal growth factor receptor (EGFR) belongs to the ErbB family of receptor tyrosine kinases (RTKs) and provides critical functions in epithelial cell physiology (Schlessinger J (2014) Cold Spring Harb Perspect Biol 6, a008912). It is frequently mutated and/or overexpressed in different types of human cancers and is the target of multiple cancer therapies currently adopted in the clinical practice (Yarden Y and Pines G (2012) Nat Rev Cancer 12, 553-563).

Accordingly, provided herein are methods for treating a subject diagnosed with (or identified as having) a cancer that include administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

Also provided herein are methods for treating a subject identified or diagnosed as having an EGFR-associated cancer that include administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof or a pharmaceutical composition thereof. In some embodiments, the subject that has been identified or diagnosed as having an EGFR-associated cancer through the use of a regulatory agency-approved, e.g., FDA-approved test or assay for identifying dysregulation of an EGFR gene, an EGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is an EGFR-associated cancer. For example, the EGFR-associated cancer can be a cancer that includes one or more EGFR inhibitor resistance mutations.

The term “regulatory agency” refers to a country's agency for the approval of the medical use of pharmaceutical agents with the country. For example, a non-limiting example of a regulatory agency is the U.S. Food and Drug Administration (FDA).

Also provided are methods for treating cancer in a subject in need thereof, the method comprising: (a) detecting an EGFR-associated cancer in the subject; and (b) administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. Some embodiments of these methods further include administering to the subject another anticancer agent (e.g., a second EGFR inhibitor, a second compound of Formula (I), or a pharmaceutically acceptable salt thereof, or an immunotherapy). In some embodiments, the subject was previously treated with a first EGFR inhibitor or previously treated with another anticancer treatment, e.g., at least partial resection of the tumor or radiation therapy. In some embodiments, the subject is determined to have an EGFR-associated cancer through the use of a regulatory agency-approved, e.g., FDA-approved test or assay for identifying dysregulation of an EGFR gene, an EGFR kinase, or expression or activity or level of any of the same, in a subject or a biopsy sample from the subject or by performing any of the non-limiting examples of assays described herein. In some embodiments, the test or assay is provided as a kit. In some embodiments, the cancer is an EGFR-associated cancer. For example, the EGFR-associated cancer can be a cancer that includes one or more EGFR inhibitor resistance mutations.

Also provided are methods of treating a subject that include performing an assay on a sample obtained from the subject to determine whether the subject has a dysregulation of an EGFR gene, an EGFR kinase, or expression or activity or level of any of the same, and administering (e.g., specifically or selectively administering) a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof to the subject determined to have a dysregulation of an EGFR gene, an EGFR kinase, or expression or activity or level of any of the same. Some embodiments of these methods further include administering to the subject another anticancer agent (e.g., a second EGFR inhibitor, a second compound of Formula (I), or a pharmaceutically acceptable salt thereof, or immunotherapy). In some embodiments of these methods, the subject was previously treated with a first EGFR inhibitor or previously treated with another anticancer treatment, e.g., at least partial resection of a tumor or radiation therapy. In some embodiments, the subject is a subject suspected of having an EGFR-associated cancer, a subject presenting with one or more symptoms of an EGFR-associated cancer, or a subject having an elevated risk of developing an EGFR-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art. In some embodiments, the dysregulation of an EGFR gene, an EGFR kinase, or expression or activity or level of any of the same includes one or more EGFR inhibitor resistance mutations.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof for use in treating an EGFR-associated cancer in a subject identified or diagnosed as having an EGFR-associated cancer through a step of performing an assay (e.g., an in vitro assay) on a sample obtained from the subject to determine whether the subject has a dysregulation of an EGFR gene, an EGFR kinase, or expression or activity or level of any of the same, where the presence of a dysregulation of an EGFR gene, an EGFR kinase, or expression or activity or level of any of the same, identifies that the subject has an EGFR-associated cancer. Also provided is the use of a compound of Formula (I), or a pharmaceutically acceptable salt thereof, for the manufacture of a medicament for treating an EGFR-associated cancer in a subject identified or diagnosed as having an EGFR-associated cancer through a step of performing an assay on a sample obtained from the subject to determine whether the subject has a dysregulation of an EGFR gene, an EGFR kinase, or expression or activity or level of any of the same where the presence of dysregulation of an EGFR gene, an EGFR kinase, or expression or activity or level of any of the same, identifies that the subject has an EGFR-associated cancer. Some embodiments of any of the methods or uses described herein further include recording in the subject's clinical record (e.g., a computer readable medium) that the subject is determined to have a dysregulation of an EGFR gene, an EGFR kinase, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. In some embodiments, the dysregulation of an EGFR gene, an EGFR kinase, or expression or activity or level of any of the same includes one or more EGFR inhibitor resistance mutations.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for use in the treatment of a cancer in a subject in need thereof, or a subject identified or diagnosed as having an EGFR-associated cancer. Also provided is the use of a compound of Formula (I), or a pharmaceutically acceptable salt thereof, for the manufacture of a medicament for treating a cancer in a subject identified or diagnosed as having an EGFR-associated cancer. In some embodiments, the cancer is an EGFR-associated cancer, for example, an EGFR-associated cancer having one or more EGFR inhibitor resistance mutations. In some embodiments, a subject is identified or diagnosed as having an EGFR-associated cancer through the use of a regulatory agency-approved, e.g., FDA-approved, kit for identifying dysregulation of an EGFR gene, an EGFR kinase, or expression or activity or level of any of the same, in a subject or a biopsy sample from the subject. As provided herein, an EGFR-associated cancer includes those described herein and known in the art.

In some embodiments of any of the methods or uses described herein, the subject has been identified or diagnosed as having a cancer with a dysregulation of an EGFR gene, an EGFR kinase, or expression or activity or level of any of the same. In some embodiments of any of the methods or uses described herein, the subject has a tumor that is positive for a dysregulation of an EGFR gene, an EGFR kinase, or expression or activity or level of any of the same. In some embodiments of any of the methods or uses described herein, the subject can be a subject with a tumor(s) that is positive for a dysregulation of an EGFR gene, an EGFR kinase, or expression or activity or level of any of the same. In some embodiments of any of the methods or uses described herein, the subject can be a subject whose tumors have a dysregulation of an EGFR gene, an EGFR kinase, or expression or activity or level of any of the same. In some embodiments of any of the methods or uses described herein, the subject is suspected of having an EGFR-associated cancer (e.g., a cancer having one or more EGFR inhibitor resistance mutations). In some embodiments, provided herein are methods for treating an EGFR-associated cancer in a subject in need of such treatment, the method comprising a) detecting a dysregulation of an EGFR gene, an EGFR kinase, or the expression or activity or level of any of the same in a sample from the subject; and b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation of an EGFR gene, an EGFR kinase, or the expression or activity or level of any of the same includes one or more EGFR kinase protein point mutations/insertions/deletions. Non-limiting examples of EGFR kinase protein point mutations/insertions/deletions are described in Table 1a and 1b. In some embodiments, the EGFR kinase protein point mutations/insertions/deletions are selected from the group consisting of G719S, G719C, G719A, L747S, D761Y, T790M, T854A, L858R, L861Q, a deletion in exon 19 (e.g., L747_A750del), and an insertion in exon 20. In some embodiments, the EGFR kinase protein point mutations/insertions/deletions are selected from the group consisting of L858R, deletions in exon 19 (e.g., L747_A750del), L747S, D761Y, T790M, and T854A. In some embodiments, the dysregulation of an EGFR gene, an EGFR kinase, or the expression or activity or level of any of the same includes one or more EGFR inhibitor resistance mutations. Non-limiting examples of EGFR inhibitor resistance mutations are described in Table 2a and 2b. In some embodiments, the EGFR inhibitor resistance mutation is a substitution at amino acid position 718, 747, 761, 790, 797, or 854 (e.g., L718Q, L747S, D761Y, T790M, C797S, and T854A). In some embodiments, the dysregulation of an EGFR gene, an EGFR kinase, or the expression or activity or level of any of the same includes one or more point mutations/insertions/deletions in exon 20. Non-limiting examples of EGFR exon 20 mutations are described in Tables 1a, 1b and 2a, 2b. In some embodiments, the EGFR exon 20 mutation is an exon 20 insertion such as V769_D770insX, D770_N771insX, N771 P772insX, P772_H773insX, and H773_V774insX. For example, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP. In some embodiments, the cancer with a dysregulation of an EGFR gene, an EGFR kinase, or expression or activity or level of any of the same is determined using a regulatory agency-approved, e.g., FDA-approved, assay or kit. In some embodiments, the tumor that is positive for a dysregulation of an EGFR gene, an EGFR kinase, or expression or activity or level of any of the same is a tumor positive for one or more EGFR inhibitor resistance mutations. In some embodiments, the tumor with a dysregulation of an EGFR gene, an EGFR kinase, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of an EGFR gene, an EGFR kinase, or expression or activity or level of any of the same (e.g., a tumor having one or more EGFR inhibitor resistance mutations). Also provided are methods of treating a subject that include administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof to a subject having a clinical record that indicates that the subject has a dysregulation of an EGFR gene, an EGFR kinase, or expression or activity or level of any of the same.

In some embodiments, the methods provided herein include performing an assay on a sample obtained from the subject to determine whether the subject has a dysregulation of an EGFR gene, an EGFR protein, or expression or level of any of the same. In some such embodiments, the method also includes administering to a subject determined to have a dysregulation of an EGFR gene, an EGFR protein, or expression or activity, or level of any of the same a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the method includes determining that a subject has a dysregulation of an EGFR gene, an EGFR protein, or expression or level of any of the same via an assay performed on a sample obtained from the subject. In such embodiments, the method also includes administering to a subject a therapeutically effective amount of a compound of Formula (I), or a pharmaceutically acceptable salt thereof. In some embodiments, the dysregulation in an EGFR gene, an EGFR kinase protein, or expression or activity or level of any of the same is one or more point mutation in the EGFR gene (e.g., any of the one or more of the EGFR point mutations described herein). The one or more point mutations in an EGFR gene can result, e.g., in the translation of an EGFR protein having one or more of the following amino acid substitutions, deletions, and insertions: G719S, G719C, G719A, L747S, D761Y, T790M, T854A, L858R, L861Q, a deletion in exon 19 (e.g., L747_A750del), and an insertion in exon 20 (e.g., V769_D770insX, D770_N771insX, N771_P772insX, P772_H773insX, and H773_V774insX). The one or more mutations in an EGFR gene can result, e.g., in the translation of an EGFR protein having one or more of the following amino acid substitutions or deletions: L858R, deletions in exon 19 (e.g., L747_A750del), L747S, D761Y, T790M, and T854A. In some embodiments, the dysregulation in an EGFR gene, an EGFR kinase protein, or expression or activity or level of any of the same is one or more EGFR inhibitor resistance mutations (e.g., any combination of the one or more EGFR inhibitor resistance mutations described herein). In some embodiments, the dysregulation in an EGFR gene, an EGFR kinase protein, or expression or activity or level of any of the same is one or more EGFR exon 20 insertions (e.g., any of the exon 20 insertions described herein). In some embodiments, the EGFR kinase protein insertion is an exon 20 insertion selected from the group consisting of: V769_D770insX, D770_N771insX, N771_P772insX, P772_H773insX, and H773_V774insX. In some embodiments, the EGFR kinase protein insertion is an exon 20 insertion selected from the group consisting of: V769_D770insX, D770_N771insX, N771_P772insX, P772_H773insX, and H773_V774insX. In some embodiments, the EGFR kinase protein insertion is an exon 20 insertion selected from the group consisting of: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772 H773insDNP, P772 H773insPNP, H773 V774insNPH, H773 V774insH, H773_V774insPH, H773_V774insAH, and P772_H773insPNP. Some embodiments of these methods further include administering to the subject another anticancer agent (e.g., a second EGFR inhibitor, a second compound of Formula (I), or a pharmaceutically acceptable salt thereof, or immunotherapy).

In some embodiments of any of the methods or uses described herein, an assay used to determine whether the subject has a dysregulation of an EGFR gene, or an EGFR kinase, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of an EGFR gene, an EGFR kinase, or expression or activity or levels of any of the same (see, e.g., the references cited herein). In some embodiments, the dysregulation of the EGFR gene, the EGFR kinase, or expression or activity or level of any of the same includes one or more EGFR inhibitor resistance mutations. In some embodiments, the sample is a biological sample or a biopsy sample (e.g., a paraffin-embedded biopsy sample) from the subject. In some embodiments, the subject is a subject suspected of having an EGFR-associated cancer, a subject having one or more symptoms of an EGFR-associated cancer, and/or a subject that has an increased risk of developing an EGFR-associated cancer).

In some embodiments, dysregulation of an EGFR gene, an EGFR kinase, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of an EGFR gene, an EGFR kinase, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of an EGFR gene, an EGFR kinase, or the expression or activity or level of any of the same. In some embodiments, liquid biopsies can be used to detect the presence of dysregulation of an EGFR gene, an EGFR kinase, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of an EGFR gene, an EGFR kinase, or the expression or activity or level of any of the same.

The term “HER2-associated disease or disorder” as used herein refers to diseases or disorders associated with or having a dysregulation of a HER2 gene, a HER2 kinase, or the expression or activity or level of any (e.g., one or more) of the same (e.g., any of the types of dysregulation of a HER2 gene, a HER2 kinase, a HER2 kinase domain, or the expression or activity or level of any of the same described herein). Non-limiting examples of a HER2-associated disease or disorder include, for example, cancer.

The term “HER2-associated cancer” as used herein refers to cancers associated with or having a dysregulation of a HER2 gene, a HER2 kinase (also called herein a HER2 protein), or expression or activity, or level of any of the same. Non-limiting examples of a HER2-associated cancer are described herein.

In some embodiments, the EGFR-associated cancer is also a HER2-associated cancer. For example, an EGFR-associated cancer can also have a dysregulation of a HER2 gene, a HER2 kinase, or the expression or activity or level of any of the same.

The phrase “dysregulation of a HER2 gene, a HER2 kinase, or the expression or activity or level of any of the same” refers to a genetic mutation (e.g., a mutation in a HER2 gene that results in the expression of a HER2 protein that includes a deletion of at least one amino acid as compared to a wild type HER2 protein, a mutation in a HER2 gene that results in the expression of a HER2 protein with one or more point mutations as compared to a wild type HER2 protein, a mutation in a HER2 gene that results in the expression of a HER2 protein with at least one inserted amino acid as compared to a wild type HER2 protein, a gene duplication that results in an increased level of HER2 protein in a cell, or a mutation in a regulatory sequence (e.g., a promoter and/or enhancer) that results in an increased level of HER2 protein in a cell), an alternative spliced version of a HER2 mRNA that results in a HER2 protein having a deletion of at least one amino acid in the HER2 protein as compared to the wild-type HER2 protein), or increased expression (e.g., increased levels) of a wild type HER2 kinase in a mammalian cell due to aberrant cell signaling and/or dysregulated autocrine/paracrine signaling (e.g., as compared to a control non-cancerous cell). As another example, a dysregulation of a HER2 gene, a HER2 protein, or expression or activity, or level of any of the same, can be a mutation in a HER2 gene that encodes a HER2 protein that is constitutively active or has increased activity as compared to a protein encoded by a HER2 gene that does not include the mutation. Non-limiting examples of HER2 kinase protein fusions and point mutations/insertions/deletions are described in Tables 3-5. Such mutation and overexpression is associated with the development of a variety of cancers (Moasser. Oncogene. 2007 Oct. 4; 26(45): 6469-6487).

Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof, are useful for treating diseases and disorders such as HER2-associated diseases and disorders, e.g., proliferative disorders such as cancers, including hematological cancers and solid tumors (e.g., advanced solid tumors).

In some embodiments, dysregulation of a HER2 gene, a HER2 kinase, or the expression or activity or level of any of the same can be caused by an activating mutation in a HER2 gene. The exemplary HER2 kinase fusions or point mutations, insertions, and deletions shown in Tables 3-5 can be caused by an activating mutation.

The term “activating mutation” in reference to HER2 describes a mutation in a HER2 gene that results in the expression of a HER2 kinase that has an increased kinase activity, e.g., as compared to a wild type HER2 kinase, e.g., when assayed under identical conditions. For example, an activating mutation can be a mutation in a HER2 gene (that results in the expression of a HER2 kinase that has one or more (e.g., two, three, four, five, six, seven, eight, nine, or ten) amino acid substitutions (e.g., any combination of any of the amino acid substitutions described herein) that has increased kinase activity, e.g., as compared to a wild type HER2 kinase, e.g., when assayed under identical conditions. In another example, an activating mutation can be a mutation in a HER2 gene that results in the expression of a HER2 kinase that has one or more (e.g., two, three, four, five, six, seven, eight, nine, or ten) amino acids deleted, e.g., as compared to a wild type HER2 kinase, e.g., when assayed under identical conditions. In another example, an activating mutation can be a mutation in a HER2 gene that results in the expression of a HER2 kinase that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type HER2 kinase, e.g., the exemplary wild type HER2 kinase described herein, e.g., when assayed under identical conditions. Additional examples of activating mutations are known in the art.

The term “wild type HER2” or “wild-type HER2 kinase” describes a HER2nucleic acid (e.g., a HER2 gene or a HER2 mRNA) or protein (e.g., a HER2 protein) that is found in a subject that does not have a HER2-associated disease, e.g., a HER2-associated cancer (and optionally also does not have an increased risk of developing a HER2-associated disease and/or is not suspected of having a HER2-associated disease), or is found in a cell or tissue from a subject that does not have a HER2-associated disease, e.g., a HER2-associated cancer (and optionally also does not have an increased risk of developing a HER2-associated disease and/or is not suspected of having a HER2-associated disease).

Provided herein is a method of treating a HER2-associated cancer (in a subject in need of such treatment, the method comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof. For example, provided herein are methods for treating a HER2-associated cancer in a subject in need of such treatment, the method comprising a) detecting a dysregulation of a HER2 gene, a HER2 kinase, or the expression or activity or level of any of the same in a sample from the subject; and b) administering a therapeutically effective amount of a compound of Formula (I), or a pharmaceutically acceptable salt thereof. In some embodiments, the dysregulation of a HER2 gene, a HER2 kinase, or the expression or activity or level of any of the same includes one or more HER2 kinase protein point mutations/insertions. Non-limiting examples of HER2 kinase protein fusions and point mutations/insertions/deletions are described in Tables 3-5. In some embodiments, the HER2 kinase protein point mutations/insertions/deletions are selected from the group consisting of S310F, S310Y, R678Q, R678W, R678P, 1767M, V773M, V777L, V842I, Y772_A775dup, A775_G776insYVMA, G776delinsVC, G776delinsVV, V777_G778insGSP, and P780_Y781insGSP. In some embodiments, the HER2 kinase protein point mutations/insertions/deletions are exon 20 point mutations/insertions/deletions selected from the group consisting of V773M, G776C, G776V, G776S, V777L, V777M, S779T, P780L, S783P, M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the HER2 kinase protein point mutations/insertions/deletions are exon 20 point mutations/insertions/deletions selected from the group consisting of Y772_A775dup, A775_G776insYVMA, G776delinsVC, G776delinsVV, V777_G778insGSP, and P780_Y781insGSP.

In some embodiments of any of the methods or uses described herein, the cancer (e.g., HER2-associated cancer) is selected from a hematological cancer (e.g., Hodgkin lymphoma, non-Hodgkin lymphoma, and leukemia such as acute-myelogenous leukemia (AML), chronic-myelogenous leukemia (CML), acute-promyelocytic leukemia, and acute lymphocytic leukemia (ALL)), alveolar rhabdomyosarcoma, central or peripheral nervous system tissue cancer, an endocrine or neuroendocrine cancer including multiple neuroendocrine type I and type II tumors, Li-Fraumeni tumors, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, tracheal cancer, oral cancer, oropharyngeal cancer, nasopharyngeal cancer, respiratory cancer, urogenital cancer, cancer of the vulva, colon cancer, esophageal cancer, cervical cancer, gastrointestinal carcinoid tumor, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer including pancreatic islet cell cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, parathyroid cancer, pituitary tumors, adrenal gland tumors, ureter cancer, biliary cancer, and urinary bladder cancer. In some embodiments, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In some embodiments, the cancer is pancreatic cancer, head and neck cancer, melanoma, colon cancer, renal cancer, leukemia, lung cancer, or breast cancer. In some cases, the cancer is melanoma, colon cancer, renal cancer, leukemia, or breast cancer.

In some such embodiments, the compounds provided herein are useful for treating a primary brain tumor or metastatic brain tumor. For example, the compounds can be used in the treatment of one or more of gliomas such as glioblastoma (also known as glioblastoma multiforme), astrocytomas, oligodendrogliomas, ependymomas, and mixed gliomas, meningiomas, medulloblastomas, gangliogliomas, schwannomas (neurilemmomas), and craniopharyngiomas (see, for example, Liu et al. J Exp Clin Cancer Res. 2019 May 23; 38(1):219); and Ding et al. Cancer Res. 2003 Mar. 1; 63(5):1106-13). In some embodiments, the brain tumor is a primary brain tumor. In some embodiments, the brain tumor is a metastatic brain tumor, e.g., a metastatic brain tumor from lung cancer, melanoma, breast cancer, ovarian cancer, colorectal cancer, kidney cancer, bladder cancer, or undifferentiated carcinoma. In some embodiments, the brain tumor is a metastatic brain tumor from lung cancer (e.g., non-small cell lung cancer). In some embodiments, the compounds provided herein exhibit brain and/or central nervous system (CNS) penetrance. In some embodiments, the patient has previously been treated with another anticancer agent, e.g., another EGFR and/or HER2 inhibitor (e.g., a compound that is not a compound of Formula I) or a multi-kinase inhibitor.

In some embodiments, the cancer is a cancer of B cell origin. In some embodiments, the cancer is a lineage dependent cancer. In some embodiments, the cancer is a lineage dependent cancer where HER2 or the dysregulation of an HER2 gene, an HER2 kinase, or expression or activity or level of any of the same, plays a role in the initiation and/or development of the cancer.

Also provided herein is a method for treating a subject diagnosed with or identified as having a HER2-associated cancer, e.g., any of the exemplary HER2-associated cancers disclosed herein, comprising administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof as defined herein.

In some embodiments, the dysregulation of a HER2 gene, a HER2 kinase, or expression or activity or level of any of the same, includes one or more deletions (e.g., deletion of an amino acid at position 12), insertions, or point mutation(s) in a HER2 kinase. In some embodiments, the dysregulation of a HER2 gene, a HER2 kinase, or expression or activity or level of any of the same, includes a deletion of one or more residues from the HER2 kinase, resulting in increased signaling activity of HER2.

In some embodiments, the dysregulation of a HER2 gene, a HER2 kinase, or expression or activity or level of any of the same, includes at least one point mutation in a HER2 gene that results in the production of a HER2 kinase that has one or more amino acid substitutions, insertions, or deletions as compared to the wild-type HER2 kinase (see, for example, the point mutations listed in Table 3). In some embodiments, dysregulation of a HER2 gene, a HER2 kinase, or expression or activity or level of any of the same, includes at least one point mutation in a HER2 gene that results in the production of a HER2 kinase that has one or more of the amino acid substitutions, insertions, or deletions in Table 3.

In some embodiments, the dysregulation of an HER2 gene, an HER2 kinase, or expression or activity or level of any of the same, includes an insertion of one or more residues in exon 20 of the HER2 gene (e.g., any of the exon 20 insertions described in Table 1a and 1b). Exon 20 of HER2 has two major regions, the c-helix (residues 770-774) and the loop following the c-helix (residues 775-783). In some embodiments, the dysregulation of an HER2 gene, an HER2 kinase, or expression or activity or level of any of the same, includes an insertion of one or more residues in exon 20 selected from the group consisting of: Y772_A775dup, A775_G776insYVMA, G776delinsVC, G776delinsVV, V777_G778insGSP, and P780_Y781insGSP.

TABLE 3
HER2 Protein Amino Acid Substitutions/Insertions/Deletions^{A}

| Amino Acid | Non-Limiting Exemplary | Non-limiting Exemplary HER2- |
|---|---|---|
| Position(s) | Mutations | associated Cancer(s) |
| 122 | P122L¹¹ | Metastatic Colorectal Cancer |
| 263 | I263T⁷ | Colorectal Cancer⁷ |
| 265 | E265K¹¹ | Metastatic Colorectal Cancer |
| 270 | A270S⁶ | Breast Cancer |
| 292 | G292R¹¹ | Metastatic Colorectal Cancer |
| 309 | G309A³, G309E¹⁵ | Breast Cancer |
| 310 | S310F⁷, S310Y⁸ | Colorectal Cancer⁷ |
| 311 | C311R⁸ | |
| 313 | L313V¹¹ | Metastatic Colorectal Cancer |
| 429 | S429R¹⁵, S429H¹⁵ | |
| 466 | A466T⁷ | Colorectal Cancer⁷ |
| 648 | A648V¹⁰ | Urinary Cancer |
| 650 | P650L¹⁰, P650S¹⁰ | Melanoma, Uterine Cancer |
| 651 | L651V¹⁰ | Breast Cancer, Cervical Cancer |
| 652 | T652M¹⁰, T652R¹⁰ | Lung Cancer, Colorectal Cancer, |
| | | Liver Cancer, Head And Neck |
| | | Cancer, Endometrial Cancer, |
| | | Ovarian Cancer |
| 653 | S653C¹⁰, S653P¹⁰ | Breast Cancer¹⁰, Urinary Cancer, |
| | | Breast Cancer, Colorectal Cancer, |
| | | Liver Cancer, Metastatic |
| | | Colorectal Cancer¹¹ |
| 654 | I654T¹⁰, I654M¹⁰, I654L¹⁰ | Lower Gastrointestinal Cancer, |
| | | Neuroendocrine Cancer, Breast |
| | | Cancer, Esophageal Cancer, Soft |
| | | Tissue Cancer |
| 655 | I655M¹⁰, I655V¹⁴ | Lung Cancer, Colorectal Cancer, |
| | | Ovarian Cancer, Urinary Cancer |
| 656 | S656C¹⁰ | Esophageal Cancer |
| 657 | A657V¹⁰ | Prostate Cancer, Colorectal Cancer |
| 659 | V659E¹⁰, V659D¹⁰, V659L¹⁰, | Lung cancer¹⁰, Biliary Cancer, |
| | V659F¹⁰, | Colorectal Cancer, Breast Cancer, |
| | | Metastatic Colorectal Cancer¹¹ |
| 659-661 | V659_I661 > VVEGI¹⁰ | Lung Cancer |
| 659-660 | V659_G660 > ER¹⁰ | Lung Cancer |
| 660 | G660D¹⁰ | Biliary Cancer¹⁰, Lung Cancer, |
| | | Urinary Cancer, Colorectal Cancer, |
| | | Glioma, Lower Gastrointestinal |
| | | Cancer, Gastric Cancer, Liver |
| | | Cancer, Metastatic Colorectal |
| | | Cancer¹¹ |
| 661 | I661V¹⁰ | Colorectal Cancer |
| 662 | L662V¹⁰ | Cervical Cancer, Lung Cancer, |
| | | Breast Cancer |
| 663 | L663P¹⁰ | Soft Tissue Cancer |
| 664 | V664F¹⁰, V664I¹⁰ | Lung Cancer, Breast Cancer, |
| | | Gastric Cancer |
| 665 | V665M¹⁰ | Prostate Cancer, Colorectal Cancer |
| 665-666 | V665_V666del¹⁰ | Breast Cancer |
| 666 | V666I¹⁰ | Colorectal Cancer |
| 667 | L667*¹⁰, L667S¹⁰ | Breast Cancer, Soft Tissue Cancer |
| 668 | G668E¹⁰, G668R¹⁰ | Glioma, Lung Cancer |
| 669 | V669A¹⁰, V669L¹⁰ | Biliary Cancer, Breast Cancer, |
| | | Glioma |
| 672 | G672R¹⁰ | SSC other, Lung Cancer |
| 673 | I673F¹⁰, I673M¹⁰, I673V¹⁰ | Colorectal Cancer, Lung Cancer |
| 674 | L674V¹⁰, L674I¹⁰ | Colorectal Cancer, Lung Cancer |
| 675 | I675M¹⁰, I675T¹⁰, I675L¹⁰ | Lung Cancer, Urinary Cancer, |
| | | Thyroid Cancer, |
| 676 | K676M¹⁰ | Lung Cancer |
| 677 | R677*¹⁰, R677L¹⁰, R677Q¹⁰ | Lung Cancer, Endometrial Cancer, |
| | | Pancreatic Cancer, Neuroendocrine |
| | | Cancer, Colorectal Cancer, |
| | | Glioma, Myeloma |
| 678 | R678Q⁷, R678W¹⁰, R678P¹⁰ | Colorectal Cancer⁷, Gastric |
| | | Cancer¹⁰, Biliary Cancer¹⁰, Urinary |
| | | Cancer¹⁰, Ovarian Cancer¹⁰, |
| | | Endometrial Cancer¹⁰, Carcinoma |
| | | of Unknown Primary¹⁰, Lung |
| | | Cancer¹⁰, Appendicidal Cancer¹⁰, |
| | | Pancreatic Cancer¹⁰, Breast Cancer¹⁰, |
| | | Neuroendocrine Cancer¹⁰, |
| | | Cervical Cancer¹⁰, Lower |
| | | Gastrointestinal Cancer¹⁰, Prostate |
| | | Cancer¹⁰, Liver Cancer¹⁰, Central |
| | | Nervous System Cancer (Non- |
| | | Glioma)¹⁰, Melanoma¹⁰, Salivary |
| | | Gland Cancer¹⁰, Metastatic |
| | | Colorectal Cancer¹¹ |
| 679 | Q679E¹⁰, Q679H¹⁰ | Pancreatic Cancer, Colorectal |
| | | Cancer |
| 680 | Q680del¹⁰ | Cervical Cancer |
| 681 | K681N¹⁰ | Lung Cancer |
| 682 | I682T¹⁰, I682M¹⁰ | Endometrial Cancer, Colorectal |
| | | Cancer |
| 683 | R683W¹⁰, R683Q¹⁰ | Breast Cancer, Pancreatic Cancer, |
| | | Endometrial Cancer, Bone Cancer, |
| | | Colorectal Cancer, |
| 684 | K684N¹⁰ | Colorectal Cancer |
| 685 | Y685H¹⁰ | Colorectal Cancer, Carcinoma of |
| | | Unknown Primary |
| 686 | T686M¹⁰, T686A¹⁰, T686R¹⁰ | Colorectal Cancer, Urinary Cancer, |
| | | Ovarian Cancer, Endometrial |
| | | Cancer |
| 688 | R688L¹⁰, R688W¹⁰, R688Q¹⁰ | Skin Cancer, Colorectal Cancer, |
| | | Melanoma |
| 689 | R689K¹⁰, R689I¹⁰ | Lung Cancer, Endometrial Cancer |
| 691 | L691R¹⁰ | Endometrial Cancer |
| 693 | E693K¹⁰, E693G¹⁰ | Ovarian Cancer, Melanoma, |
| | | Colorectal Cancer, Breast Cancer, |
| | | Carcinoma of Unknown Primary |
| 694 | T694M¹⁰, T694S¹⁰ | Carcinoma of Unknown Primary, |
| | | Colorectal Cancer, Pancreatic |
| | | Cancer |
| 695 | E695K¹⁰ | Melanoma, Urinary Cancer |
| 697 | V697L¹⁰, V697M¹⁰, | Breast Cancer, Lung Cancer, |
| | V697del¹⁰ | Ovarian Cancer, Lower |
| | | Gastrointestinal Cancer, Colorectal |
| | | Cancer, Skin Cancer, Pancreatic |
| | | Cancer, Salivary Gland Cancer, |
| | | Carcinoma of Unknown Primary, |
| | | Cervical Cancer, Endometrial |
| | | Cancer, Gastric Cancer, |
| 699 | P699del¹⁰, P699S | Endometrial Cancer |
| 701 | T701I¹⁰ | Ovarian Cancer |
| 702 | P702S¹⁰, P702L¹⁰ | Gastric Cancer, Lower |
| | | Gastrointestinal Cancer, |
| | | Carcinoma of Unknown Primary, |
| | | Endometrial Cancer, Breast |
| | | Cancer, Ovarian Cancer, Liver |
| | | Cancer |
| 704 | G704E¹⁰, G704R¹⁰ | Glioma, Colorectal Cancer |
| 705 | A705V¹⁰ | Colorectal Cancer, Soft Tissue |
| | | Cancer |
| 706 | M706V¹⁰ | Breast Cancer |
| 707 | P707L¹⁰ | Soft Tissue Cancer |
| 709 | Q709L¹⁰, Q709K¹⁰ | Glioma, Lung Cancer, Lower |
| | | Gastrointestinal Cancer |
| 710 | A710V¹⁰ | Lung Cancer |
| 711 | Q711H¹⁰ | Breast Cancer, Lung Cancer |
| 712 | M712L¹⁰ | Neuroendocrine Cancer, |
| | | Esophageal Cancer |
| 713 | R713Q¹⁰, R713W¹⁰, R713L¹⁰ | Prostate Cancer, Lung Cancer, |
| | | Endometrial Cancer |
| 726 | L726I¹⁵, L726F¹⁵ | |
| 733 | 733I¹¹ | Metastatic Colorectal Cancer¹¹ |
| 755 | L755S^{2,7}, L755W³, L755P⁸, | Lung Adenocarcinoma², Breast |
| | L755F¹⁴ | Cancer³, Bone Metastases From |
| | | Breast Cancer⁵, Colorectal |
| | | Cancer⁷, Metastatic Colorectal |
| | | Cancer¹¹ |
| 755-759 | del. 755-759³ | Breast Cancer³ |
| 760 | S760A¹⁵ | |
| 767 | I767M¹¹ | Metastatic Colorectal Cancer¹¹ |
| 769 | D769H², D769Y³, D769N¹¹ | Lung Adenocarcinoma², Breast |
| | | Cancer³, Metastatic Colorectal |
| | | Cancer¹¹ |
| 773 | V773M¹¹ | Metastatic Colorectal Cancer¹¹ |
| 776 | G776C², G776V⁸, G776S¹¹ | Lung Adenocarcinoma, Metastatic |
| | | Colorectal Cancer¹¹ |
| 777 | V777L^{2,7}, V777M² | Lung Adenocarcinoma², Breast |
| | | Cancer³, Bone Metastases From |
| | | Breast Cancer⁵, Colorectal |
| | | Cancer⁷, Metastatic Colorectal |
| | | Cancer¹¹ |
| 779 | S779T⁸ | Breast Cancer |
| 780 | P780ins³, P780L¹⁵ | Breast Cancer³ |
| 783 | S783P2 | Breast Cancer |
| 784 | R784G¹² | Metastatic Colorectal Cancer¹² |
| 785 | L785F¹⁵ | Breast Cancer |
| 798 | T798I⁴, T798M¹⁵ | Breast Cancer |
| 835 | Y835F¹⁵ | |
| 838 | R838Q¹⁵ | |
| 842 | V842I^{3,7} | Breast Cancer³, Colorectal |
| | | Cancer⁷, Metastatic Colorectal |
| | | Cancer¹¹ |
| 862 | T862A¹¹ | Metastatic Colorectal Cancer¹¹ |
| 866 | 866M⁷ | Colorectal Cancer⁷ |
| 868 | R868W⁷ | Colorectal Cancer⁷ |
| 869 | L869R⁴ | Breast Cancer |
| 869 + 798 | L869R + T798I⁴ | Breast Cancer |
| 878 | H878Y¹¹ | Metastatic Colorectal Cancer¹¹ |
| 887 | M887I¹⁵ | |
| 896 | R896C³, R896H¹¹ | Breast Cancer³, Metastatic |
| | | Colorectal Cancer¹¹ |
| 896 + 755 | R896C + L755W³ | Breast Cancer |
| 1136 | Q1136fs*5¹¹ | Metastatic Colorectal Cancer¹¹ |
| 1170 | P1170fs*88+¹¹ | Metastatic Colorectal Cancer¹¹ |
| 1189 | G1189fs*9¹¹ | Metastatic Colorectal Cancer¹¹ |
| 1201 | G120IV¹⁵ | |
| 1219 | N1219S⁷ | Colorectal Cancer⁷ |
| 1232 | A1232fs*25+¹¹ | Metastatic Colorectal Cancer¹¹ |
| Exon 16 | Deletion¹¹ | Metastatic Colorectal Cancer¹¹ |

Exon 20 deletions and insertions

| | |
|---|---|
| M774AYVM¹³ | Non-Small Cell Lung Cancer¹³ |
| M774delinsWLV⁹ | Non-Small Cell Lung Cancer⁹ |
| A775_G776insYVMA (c. | Lung Adenocarcinoma |
| 2324_2325ins12)¹ | |
| A775_G776insAVMA (c. | Lung Adenocarcinoma |
| 2324_2325ins12)¹⁴ | |
| A775_G776insSVMA⁹ | Non-Small Cell Lung Cancer⁹ |
| A775_G776insVAG¹⁴ | Lung Adenocarcinoma |
| A775insV G776C⁸ | |
| A775_G776insI⁹ | Non-Small Cell Lung Cancer⁹ |
| G776delinsVC^{2,8}; | Lung Adenocarcinoma |
| G776delinsVV⁸; G776delinsLC⁹ | |
| G776C V777insC⁸; G776C | |
| V777insV⁸ | |
| V777_G778insCG², | Lung Adenocarcinoma², Non- |
| V777_G778insGSP¹⁶ | Small Cell Lung Cancer¹⁶ |
| G778_S779insCPG⁹ | Non-Small Cell Lung Cancer⁹ |
| P780_Y781insGSP^{2,8} | Lung Adenocarcinoma |

^{A}The HER2 mutations shown may be activating mutations and/or confer increased resistance of HER2 to a HER2 inhibitor and/or a multi-kinase inhibitor (MKI), e.g., as compared to a wildtype HER2.
¹Li et al. J Thorac Oncol. 2016 March; 11(3): 414-9.
²Arcila et al. Clin Cancer Res. 2012 Sep. 15; 18(18): 10.1158/1078-0432.CCR-12-0912.
³Bose et al. Cancer Discov. 2013 February; 3(2): 224-37.
⁴Hanker et al. Cancer Discov. 2017 June; 7(6): 575-585.
⁵Christgen et al. Virchows Arch. 2018 November; 473(5): 577-582.
⁶Si et al. Cancer Biomark. 2018; 23(2): 165-171.
⁷Kavuri et al. Cancer Discov. 2015 August; 5(8): 832-841.
⁸Robichaux et al. Nat Med. 2018 May; 24(5): 638-646.
⁹Kosaka et al. Cancer Res. 2017 May. 15; 77(10): 2712-2721.
¹⁰Pahuja et al. Cancer Cell. 2018 Nov. 12; 34(5): 792-806.e5.
¹¹Ross et al. Cancer. 2018 Apr. 1; 124(7): 1358-1373.
¹²Gharib et al. J Cell Physiol. 2019 August; 234(8): 13137-13144.
¹³Krawczyk et al. Oncol Lett. 2013 October; 6(4): 1063-1067.
¹⁴Lai et al. Eur J Cancer. 2019 March, 109: 28-35.
¹⁵Sun et al. J Cell Mol Med. 2015 December; 19(12): 2691-2701.
¹⁶Xu et al. Thorac Cancer. 2020 March; 11(3): 679-685.

In some embodiments, the dysregulation of a HER2 gene, a HER2 kinase, or expression or activity or level of any of the same, includes a splice variation in a HER2 mRNA which results in an expressed protein that is an alternatively spliced variant of HER2 having at least one residue deleted (as compared to the wild-type HER2 kinase) resulting in a constitutive activity of a HER2 kinase domain. In some embodiments, the splice variant of HER2 is Δ 16HER-3 or p95HER-2. See, e.g., Sun et al. J Cell Mol Med. 2015 December; 19(12): 2691-2701.

In some embodiments, dysregulation of an HER2 gene, an HER2 kinase, or the expression or activity or level of any of the same can be caused by a splice variation in a HER2 mRNA that results in the expression of an altered HER2 protein that has increased resistance to inhibition by an HER2 inhibitor, a tyrosine kinase inhibitor (TKI), and/or a multi-kinase inhibitor (MKI), e.g., as compared to a wild type HER2 kinase (e.g., the HER2 variants described herein). See, e.g., Rexer and Arteaga. Crit Rev Oncog. 2012; 17(1): 1-16.

In some embodiments, the dysregulation of a HER2 gene, a HER2 kinase, or expression or activity or level of any of the same, includes one or more chromosome translocations or inversions resulting in HER-2 gene fusions, respectively. In some embodiments, the dysregulation of a HER2 gene, a HER2 kinase, or expression or activity or level of any of the same, is a result of genetic translocations in which the expressed protein is a fusion protein containing residues from a non-HER2 partner protein and HER2, and include a minimum of a functional HER2 kinase domain, respectively.

TABLE 4
Exemplary HER2 Fusion Proteins and Cancers

| Non-limiting Exemplary | Non-limiting Exemplary |
|---|---|
| HER2 Fusions | HER2-associated Cancer(s) |
| ZNF207_ex2/HER2_ex18¹ | Gastric Cancer |
| MDK_ex4/HER2_ex11¹ | Gastric Cancer |
| NOS2_ex2/HER2_ex2¹ | Gastric Cancer |

¹Yu et al. J Transl Med. 2015: 13: 116.

In some embodiments, the dysregulation of a HER2 gene, a HER2 kinase, or expression or activity or level of any of the same, includes at least one point mutation in a HER2 gene that results in the production of a HER2 kinase that has one or more amino acid substitutions or insertions or deletions in a HER2 gene that results in the production of a HER2 kinase that has one or more amino acids inserted or removed, as compared to the wild-type HER2 kinase.

In other embodiments, the dysregulation of a HER2 gene, a HER2 kinase, or expression or activity or level of any of the same, includes at least one point mutation in a HER2 gene that results in the production of a HER2 kinase that has one or more amino acid substitutions as compared to the wild-type HER2 kinase, and which has increased resistance to a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as compared to a wild type HER2 kinase or a HER2 kinase not including the same mutation.

Exemplary Sequence of Mature Human HER2 Protein (UniProtKB entry P04626)
(SEQ ID NO: 2)
MELAALCRWG LLLALLPPGA ASTQVCIGTD MKLRLPASPE THLDMLRHLY
QGCQVVQGNL ELTYLPTNAS LSFLQDIQEV QGYVLIAHNQ VRQVPLQRLR
IVRGTQLFED NYALAVLDNG DPLNNTTPVT GASPGGLREL QLRSLTEILK
GGVLIQRNPQ LCYQDTILWK DIFHKNNQLA LTLIDTNRSR ACHPCSPMCK
GSRCWGESSE DCQSLTRTVC AGGCARCKGP LPTDCCHEQC AAGCTGPKHS
DCLACLHENH SGICELHCPA LVTYNTDTFE SMPNPEGRYT FGASCVTACP
YNYLSTDVGS CTLVCPLHNQ EVTAEDGTQR CEKCSKPCAR VCYGLGMEHL
REVRAVTSAN IQEFAGCKKI FGSLAFLPES EDGDPASNTA PLQPEQLQVF
ETLEEITGYL YISAWPDSLP DLSVFQNLQV IRGRILHNGA YSLTLQGLGI
SWLGLRSLRE LGSGLALIHH NTHLCFVHTV PWDQLERNPH QALLHTANRP
EDECVGEGLA CHQLCARGHC WGPGPTQCVN CSQFLRGQEC VEECRVLQGL
PREYVNARHC LPCHPECQPQ NGSVTCFGPE ADQCVACAHY KDPPFCVARC
PSGVKPDLSY MPIWKFPDEE GACQPCPINC THSCVDLDDK GCPAEQRASP
LTSIISAVVG ILLVVVLGVV FGILIKRRQQ KIRKYTMRRL LQETELVEPL
TPSGAMPNQA QMRILKETEL RKVKVIGSGA FGTVYKGIWI PDGENVKIPV
AIKVLRENTS PKANKEILDE AYVMAGVGSP YVSRLLGICL TSTVQLVTQL
MPYGCLLDHV RENRGRLGSQ DLLNWCMQIA KGMSYLEDVR LVHRDLAARN
VLVKSPNHVK ITDFGLARLL DIDETEYHAD GGKVPIKWMA LESILRRRFT
HQSDVWSYGV TVWELMTFGA KPYDGIPARE IPDLLEKGER LPQPPICTID
VYMIMVKCWM IDSECRPRER ELVSEFSRMA RDPQRFVVIQ NEDLGPASPL
DSTFYRSLLE DDDMGDLVDA EEYLVPQQGF FCPDPAPGAG GMVHHRHRSS
STRSGGGDLT LGLEPSEEEA PRSPLAPSEG AGSDVEDGDL GMGAAKGLQS
LPTHDPSPLQ RYSEDPTVPL PSETDGYVAP LTCSPQPEYV NQPDVRPQPP
SPREGPLPAA RPAGATLERP KTLSPGKNGV VKDVFAFGGA VENPEYLTPQ
GGAAPQPHPP PAFSPAFDNL YYWDQDPPER GAPPSTEKGT PTAENPEYLG
LDVPV

In some embodiments, dysregulation of an HER2 gene, an HER2 kinase, or expression or activity or level of any of the same, includes at least one HER2 inhibitor resistance mutation in an HER2 gene that results in the production of an HER2 kinase that has one or more of the amino acid substitutions, insertions, or deletions as described in Table 5. In some embodiments, compounds of Formula (I) (e.g., Formula (I-a), (I-b), (I-c), (I-d), (I-e), (I-f), (I-g), (I-h), (I-i), (I-j), or (I-k)) and pharmaceutically acceptable salts and solvates thereof, are useful in treating subjects that develop cancers with HER2 inhibitor resistance mutations (e.g., that result in an increased resistance to a first HER2 inhibitor, e.g., a substitution at amino acid position 755 or 798 (e.g., L755S, L755P, T798I, and T798M), and/or one or more HER2 inhibitor resistance mutations listed in Table 5) by either dosing in combination or as a subsequent or additional (e.g., follow-up) therapy to existing drug treatments (e.g., other inhibitors of HER2; e.g., first and/or second HER2 inhibitors).

TABLE 5
HER2 Protein Amino Acid Resistance Mutations

| Amino Acid | | Non-limiting Exemplary |
|---|---|---|
| Position(s) | Non-Limiting Exemplary Mutations | HER2-associated Cancer(s) |
| 726 | L726I, L726F² | Breast Cancer |
| 755 | L755S², L755P² | Breast Cancer |
| 780 | P780L² | Breast Cancer |
| 783 | S783P² | Breast Cancer |
| 785 | L785F² | Breast Cancer |
| 798 | T798I¹, T798M² | Breast Cancer |

¹Hanker et al. Cancer Discov. 2017 June; 7(6): 575-585.
²Sun et al. J Cell Mol Med. 2015 December; 19(12): 2691-2701.

As used herein, a “first inhibitor of HER2” or “first HER2 inhibitor” is a HER2 inhibitor as defined herein, but which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof as defined herein. As used herein, a “second inhibitor of HER2” or a “second HER2 inhibitor” is a HER2 inhibitor as defined herein, but which does not include a compound of Formula (I), or a pharmaceutically acceptable salt thereof, as defined herein. When both a first and a second inhibitor of HER2 are present in a method provided herein, the first and second inhibitors of HER2 are different. In some embodiments, the first and/or second inhibitor of HER2 bind in a different location than a compound of Formula (I). For example, in some embodiments, a first and/or second inhibitor of HER2 can inhibit dimerization of HER2, while a compound of Formula (I) can inhibit the active site. In some embodiments, a first and/or second inhibitor of HER2 can be an allosteric inhibitor of HER2, while a compound of Formula (I) can inhibit the HER2 active site.

Exemplary first and second inhibitors of HER2 are described herein. In some embodiments, a first or second inhibitor of HER2 can be selected from the group consisting of: trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

In some embodiments, compounds of Formula (I) (e.g., Formula (I-a), (I-b), (I-c), (I-d), (I-e), (I-f), (I-g), (I-h), (I-i), (I-j), or (I-k)), or pharmaceutically acceptable salts and solvates thereof, are useful for treating a cancer that has been identified as having one or more HER2 inhibitor resistance mutations (that result in an increased resistance to a first or second inhibitor of HER2, e.g., a substitution described in Table 5 including substitutions at amino acid position 755 or 798 (e.g., L755S, L755P, T798I, and T798M)). In some embodiments, the one or more HER2 inhibitor resistance mutations occurs in a nucleic acid sequence encoding a mutant HER2 protein (e.g., a mutant HER2 protein having any of the mutations described in Table 3) resulting in a mutant HER2 protein that exhibits HER2 inhibitor resistance.

Like EGFR, the epidermal growth factor receptor 2 (HER2) belongs to the ErbB family of receptor tyrosine kinases (RTKs) and provides critical functions in epithelial cell physiology (Schlessinger J (2014) Cold Spring Harb Perspect Biol 6, a008912; and Moasser. Oncogene. 2007 Oct. 4; 26(45): 6469-6487). It is frequently mutated and/or overexpressed in different types of human cancers and is the target of multiple cancer therapies currently adopted in the clinical practice (Moasser. Oncogene. 2007 Oct. 4; 26(45): 6469-6487).

Accordingly, provided herein are methods for treating a subject identified or diagnosed as having a HER2-associated cancer that include administering to the subject a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof or a pharmaceutical composition thereof. In some embodiments, the subject that has been identified or diagnosed as having a HER2-associated cancer through the use of a regulatory agency-approved, e.g., FDA-approved test or assay for identifying dysregulation of a HER2 gene, a HER2 kinase, or expression or activity or level of any of the same, in a subject or a biopsy sample from the subject or by performing any of the non-limiting examples of assays described herein. In some embodiments, the test or assay is provided as a kit. In some embodiments, the cancer is a HER2-associated cancer. Also provided are methods for treating cancer in a subject in need thereof, the method comprising. (a) detecting a HER2-associated cancer in the subject; and (b) administering to the subject a therapeutically effective amount of a compound of Formula (I), or a pharmaceutically acceptable salt thereof or a pharmaceutical composition thereof. Some embodiments of these methods further include administering to the subject another anticancer agent (e.g., a second HER2 inhibitor, a second compound of Formula (I), or a pharmaceutically acceptable salt thereof, or an immunotherapy). In some embodiments, the subject was previously treated with a first HER2 inhibitor or previously treated with another anticancer treatment, e.g., at least partial resection of the tumor or radiation therapy. In some embodiments, the subject is determined to have a HER2-associated cancer through the use of a regulatory agency-approved, e.g., FDA-approved test or assay for identifying dysregulation of a HER2 gene, a HER2 kinase, or expression or activity or level of any of the same, in a subject or a biopsy sample from the subject or by performing any of the non-limiting examples of assays described herein. In some embodiments, the test or assay is provided as a kit. In some embodiments, the cancer is a HER2-associated cancer.

Also provided are methods of treating a subject that include performing an assay on a sample obtained from the subject to determine whether the subject has a dysregulation of a HER2 gene, a HER2 kinase, or expression or activity or level of any of the same, and administering (e.g., specifically or selectively administering) a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof to the subject determined to have a dysregulation of a HER2 gene, a HER2 kinase, or expression or activity or level of any of the same. Some embodiments of these methods further include administering to the subject another anticancer agent (e.g., a second HER2 inhibitor, a second compound of Formula (I), or a pharmaceutically acceptable salt thereof, or immunotherapy). In some embodiments of these methods, the subject was previously treated with a first HER2 inhibitor or previously treated with another anticancer treatment, e.g., at least partial resection of a tumor or radiation therapy. In some embodiments, the subject is a subject suspected of having a HER2-associated cancer, a subject presenting with one or more symptoms of a HER2-associated cancer, or a subject having an elevated risk of developing a HER2-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

As used herein, a “first inhibitor of HER2” or “first HER2 inhibitor” is a HER2 inhibitor as defined herein, which does not include a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof as defined herein. As used herein, a “second inhibitor of HER2” or a “second HER2 inhibitor” is an inhibitor of HER2 as defined herein, which does not include a compound of Formula (I), or a pharmaceutically acceptable salt thereof as defined herein. When both a first and a second HER2 inhibitor are present in a method provided herein, the first and second HER2 inhibitors are different.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof for use in treating a HER2-associated cancer in a subject identified or diagnosed as having a HER2-associated cancer through a step of performing an assay (e.g., an in vitro assay) on a sample obtained from the subject to determine whether the subject has a dysregulation of a HER2 gene, a HER2 kinase, or expression or activity or level of any of the same, where the presence of a dysregulation of a HER2 gene, a HER2 kinase, or expression or activity or level of any of the same, identifies that the subject has a HER2-associated cancer. Also provided is the use of a compound of Formula (I), or a pharmaceutically acceptable salt thereof, for the manufacture of a medicament for treating a HER2-associated cancer in a subject identified or diagnosed as having a HER2-associated cancer through a step of performing an assay on a sample obtained from the subject to determine whether the subject has a dysregulation of a HER2 gene, a HER2 kinase, or expression or activity or level of any of the same where the presence of dysregulation of a HER2 gene, a HER2 kinase, or expression or activity or level of any of the same, identifies that the subject has a HER2-associated cancer. Some embodiments of any of the methods or uses described herein further include recording in the subject's clinical record (e.g., a computer readable medium) that the subject is determined to have a dysregulation of a HER2 gene, a HER2 kinase, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, for use in the treatment of a cancer in a subject in need thereof, or a subject identified or diagnosed as having a HER2-associated cancer. Also provided is the use of a compound of Formula (I), or a pharmaceutically acceptable salt thereof, for the manufacture of a medicament for treating a cancer in a subject identified or diagnosed as having a HER2-associated cancer (In some embodiments, a subject is identified or diagnosed as having a HER2-associated cancer through the use of a regulatory agency-approved, e.g., FDA-approved, kit for identifying dysregulation of a HER2 gene, a HER2 kinase, or expression or activity or level of any of the same, in a subject or a biopsy sample from the subject. As provided herein, a HER2-associated cancer includes those described herein and known in the art.

In some embodiments of any of the methods or uses described herein, the subject has been identified or diagnosed as having a cancer with a dysregulation of a HER2 gene, a HER2 kinase, or expression or activity or level of any of the same. In some embodiments of any of the methods or uses described herein, the subject has a tumor that is positive for a dysregulation of a HER2 gene, a HER2 kinase, or expression or activity or level of any of the same. In some embodiments of any of the methods or uses described herein, the subject can be a subject with a tumor(s) that is positive for a dysregulation of a HER2 gene, a HER2 kinase, or expression or activity or level of any of the same. In some embodiments of any of the methods or uses described herein, the subject can be a subject whose tumors have a dysregulation of a HER2 gene, a HER2 kinase, or expression or activity or level of any of the same. In some embodiments of any of the methods or uses described herein, the subject is suspected of having a HER2-associated cancer. In some embodiments, provided herein are methods for treating a HER2-associated cancer in a subject in need of such treatment, the method comprising a) detecting a dysregulation of a HER2 gene, a HER2 kinase, or the expression or activity or level of any of the same in a sample from the subject; and b) administering a therapeutically effective amount of a compound of Formula (I)(e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the dysregulation of a HER2 gene, a HER2 kinase, or the expression or activity or level of any of the same includes one or more HER2 kinase protein point mutations/insertions/deletions. Non-limiting examples of HER2 kinase protein fusions and point mutations/insertions/deletions are described in Tables 3-5. In some embodiments, the HER2 kinase protein point mutations/insertions/deletions are selected from the group consisting of a point mutation at amino acid position 310, 678, 755, 767, 773, 777, or 842 (e.g., S310F, S310Y, R678Q, R678W, R678P, I767M, V773M, V777L, and V842I) and/or an insertion or deletion at amino acid positions 772, 775, 776, 777, and 780 (e.g., Y772_A775dup, A775_G776insYVMA, G776delinsVC, G776delinsVV, V777_G778insGSP, and P780_Y781insGSP). In some embodiments, the HER2 kinase protein point mutation/insertion/deletion is an exon 20 point mutation/insertion/deletion. In some embodiments, the HER2 exon 20 point mutation/insertion/deletion is a point mutation at amino acid position 773, 776, 777, 779, 780, and 783 (e.g., V773M, G776C, G776V, G776S, V777L, V777M, S779T, P780L, and S783P) and/or an exon 20 insertion/deletion such as an insertion/deletion at amino acid positions 774, 775, 776, 777, 778, and 780. In some embodiments, the HER2 kinase protein insertion is an exon 20 insertion selected from the group consisting of: A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the HER2 kinase protein mutation/insertion/deletion is an exon 20 insertion/deletion selected from the group consisting of: is Y772_A775dup, A775_G776insYVMA, G776delinsVC, G776delinsVV, V777_G778insGSP, or P780_Y781insGSP. In some embodiments, the cancer with a dysregulation of a HER2 gene, a HER2 kinase, or expression or activity or level of any of the same is determined using a regulatory agency-approved, e.g., FDA-approved, assay or kit. In some embodiments, the tumor that is positive for a dysregulation of a HER2 gene, a HER2 kinase, or expression or activity or level of any of the same is a tumor positive for one or more HER2 inhibitor resistance mutations. In some embodiments, the tumor with a dysregulation of a HER2 gene, a HER2 kinase, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of a HER2 gene, a HER2 kinase, or expression or activity or level of any of the same. Also provided are methods of treating a subject that include administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, to a subject having a clinical record that indicates that the subject has a dysregulation of a HER2 gene, a HER2 kinase, or expression or activity or level of any of the same.

In some embodiments, the methods provided herein include performing an assay on a sample obtained from the subject to determine whether the subject has a dysregulation of a HER2 gene, a HER2 kinase, or expression or level of any of the same. In some such embodiments, the method also includes administering to a subject determined to have a dysregulation of a HER2 gene, a HER2 kinase, or expression or activity, or level of any of the same a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the method includes determining that a subject has a dysregulation of a HER2 gene, a HER2 kinase, or expression or level of any of the same via an assay performed on a sample obtained from the subject. In such embodiments, the method also includes administering to a subject a therapeutically effective amount of a compound of Formula (I), or a pharmaceutically acceptable salt thereof. In some embodiments, the dysregulation in a HER2 gene, a HER2 kinase, or expression or activity or level of any of the same is one or more point mutation in the HER2 gene (e.g., any of the one or more of the HER2 point mutations described herein). The one or more point mutations in a HER2 gene can result, e.g., in the translation of a HER2 protein having one or more of the following amino acid substitutions: S310F, S310Y, R678Q, R678W, R678P, I767M, V773M, V777L, and V842I. The one or more point mutations in a HER2 gene can result, e.g., in the translation of a HER2 protein having one or more of the following exon 20 amino acid substitutions: V773M, G776C, G776V, G776S, V777L, V777M, S779T, P780L, and S783P. In some embodiments, the dysregulation in a HER2 gene, a HER2 kinase, or expression or activity or level of any of the same is one or more insertions in the HER2 gene (e.g., any of the one or more of the HER2 insertions described herein). The one or more insertions in a HER2 gene can result, e.g., in the translation of a HER2 protein having one or more of the following exon 20 insertions: M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777_G778insCG, G778_S779insCPG, and P780_Y781insGSP. In some embodiments, the one or more insertions in a HER2 gene can result, e.g., in the translation of a HER2 protein having one or more of the following exon 20 insertions: Y772_A775dup, A775_G776insYVMA, G776delinsVC, G776delinsVV, V777_G778insGSP, and P780_Y781insGSP. Some embodiments of these methods further include administering to the subject another anticancer agent (e.g., a second HER2 inhibitor, a second compound of Formula (I), or a pharmaceutically acceptable salt thereof, or immunotherapy).

In some embodiments of any of the methods or uses described herein, an assay used to determine whether the subject has a dysregulation of a HER2 gene, a HER2 kinase, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a HER2 gene, a HER2 kinase, or expression or activity or levels of any of the same (see, e.g., the references cited herein). In some embodiments, the sample is a biological sample or a biopsy sample (e.g., a paraffin-embedded biopsy sample) from the subject. In some embodiments, the subject is a subject suspected of having a HER2-associated cancer, a subject having one or more symptoms of a HER2-associated cancer, and/or a subject that has an increased risk of developing a HER2-associated cancer.

In some embodiments, dysregulation of a HER2 gene, a HER2 kinase, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of a HER2 gene, a HER2 kinasev, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a HER2 gene, a HER2 kinase, or the expression or activity or level of any of the same. In some embodiments, liquid biopsies can be used to detect the presence of dysregulation of a HER2 gene, a HER2 kinase, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a HER2 gene, a HER2 kinase, or the expression or activity or level of any of the same.

Also provided is a method for inhibiting EGFR activity in a cell, comprising contacting the cell with a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. Also provided is a method for inhibiting HER2 activity in a cell, comprising contacting the cell with a compound of Formula (I), or a pharmaceutically acceptable salt thereof. Further provided herein is a method for inhibiting EGFR and HER2 activity in a cell, comprising contacting the cell with a compound of Formula (I), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof, to a subject having a cell having aberrant EGFR activity and/or HER2 activity. In some embodiments, the cell is a cancer cell. In some embodiments, the cancer cell is any cancer as described herein. In some embodiments, the cancer cell is an EGFR-associated cancer cell. In some embodiments, the cancer cell is a HER2-associated cancer cell. As used herein, the term “contacting” refers to the bringing together of indicated moieties in an in vitro system or an in vivo system. For example, “contacting” an EGFR kinase with a compound provided herein includes the administration of a compound provided herein to an individual or subject, such as a human, having an EGFR kinase, as well as, for example, introducing a compound provided herein into a sample containing a cellular or purified preparation containing the EGFR kinase.

Also provided herein is a method of inhibiting cell proliferation, in vitro or in vivo, the method comprising contacting a cell with an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof as defined herein.

Further provided herein is a method of increase cell death, in vitro or in vivo, the method comprising contacting a cell with an effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or a pharmaceutical composition thereof as defined herein. Also provided herein is a method of increasing tumor cell death in a subject. The method comprises administering to the subject an effective compound of Formula (I), or a pharmaceutically acceptable salt thereof, in an amount effective to increase tumor cell death.

The phrase “therapeutically effective amount” means an amount of compound that, when administered to a subject in need of such treatment, is sufficient to (i) treat an EGFR kinase-associated disease or disorder or a HER2 kinase-associated disease or disorder, (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, the compounds of Formula (I) (e.g., Formula (I-c), (I-d), (I-e), (I-f), (I-g), (I-h), (I-i), (I-j), or (I-k)), including pharmaceutically acceptable salts or solvates thereof, can be administered in the form of pharmaceutical compositions as described herein.

Also provided herein is a method of treating a subject having a cancer, wherein the method comprises:
(a) determining that a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a therapeutically effective amount of a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject.

Further provided herein is a method of treating a subject having a cancer, wherein the method comprises:
(a) determining that a cancer cell in a sample obtained from a subject having a cancer and previously administered one or more doses of a first EGFR inhibitor does not have one or more EGFR inhibitor resistance mutations that confer increased resistance to a cancer cell or tumor to treatment with the first EGFR inhibitor that was previously administered to the subject; and
(b) administering additional doses of the first EGFR inhibitor to the subject.

Combinations

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other kinase inhibitors, signal transduction inhibitors and/or monoclonal antibodies. For example, a surgery may be open surgery or minimally invasive surgery. Compounds of Formula (I) (e.g., Formula (I-a), (I-b), (I-c), (I-d), (I-e), (I-f), (I-g), (I-h), (I-i), (I-j), or (I-k)), or pharmaceutically acceptable salts or solvates thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof, for a period of time and then undergo at least partial resection of the tumor. In some embodiments, the treatment with one or more doses of a compound of Formula (I), or a pharmaceutically acceptable salt thereof, reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof, for a period of time and under one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent, such as a first EGFR inhibitor, a first HER2 inhibitor, or a multi-kinase inhibitor, immunotherapy, or radiation (e.g., radioactive iodine)). In some embodiments, a subject has a cancer (e.g., a locally advanced or metastatic tumor) that is refractory or intolerant to prior therapy (e.g., administration of a chemotherapeutic agent, such as a first EGFR inhibitor, a first HER2 inhibitor, or a multi-kinase inhibitor, immunotherapy, or radiation (e.g., radioactive iodine)). In some embodiments, a subject has a cancer (e.g., a locally advanced or metastatic tumor) that has no standard therapy. In some embodiments, a subject is EGFR inhibitor naïve. For example, the subject is naïve to treatment with a selective EGFR inhibitor. In some embodiments, a subject is not EGFR inhibitor naïve. In some embodiments, a subject is HER2 inhibitor naïve. For example, the subject is naïve to treatment with a selective HER2 inhibitor. In some embodiments, a subject is not HER2 inhibitor naïve. In some embodiments, a subject has undergone prior therapy. For example, treatment with a multi-kinase inhibitor (MKI), an EGFR tyrosine kinase inhibitor (TKI), osimertinib, gefitinib, erlotinib, afatinib, lapatinib, neratinib, AZD-9291, CL-387785, CO-1686, or WZ4002.

In some embodiments of any the methods described herein, the compound of Formula (I) (e.g., Formula (I-a), (I-b), (I-c), (I-d), (I-e), (I-f), (I-g), (I-h), (I-i), (I-j), or (I-k)) (or a pharmaceutically acceptable salt thereof) is administered in combination with a therapeutically effective amount of at least one additional therapeutic agent selected from one or more additional therapies or therapeutic (e.g., chemotherapeutic) agents.

Non-limiting examples of additional therapeutic agents include: other EGFR-targeted therapeutic agents (i.e., a first or second EGFR inhibitor), other HER2-targeted therapeutic agents (i.e., a first or second HER2 inhibitor), RAS pathway targeted therapeutic agents, PARP inhibitors, other kinase inhibitors (e.g., receptor tyrosine kinase-targeted therapeutic agents (e.g., Trk inhibitors or multi-kinase inhibitors)), farnesyl transferase inhibitors, signal transduction pathway inhibitors, checkpoint inhibitors, modulators of the apoptosis pathway (e.g., obatoclax); cytotoxic chemotherapeutics, angiogenesis-targeted therapies, immune-targeted agents, including immunotherapy, and radiotherapy.

In some embodiments, the other EGFR-targeted therapeutic is a multi-kinase inhibitor exhibiting EGFR inhibition activity. In some embodiments, the other EGFR-targeted therapeutic inhibitor is selective for an EGFR kinase.

Non-limiting examples of EGFR-targeted therapeutic agents (e.g., a first EGFR inhibitor or a second EGFR inhibitor) include an EGFR-selective inhibitor, a panHER inhibitor, and an anti-EGFR antibody. In some embodiments, the EGFR inhibitor is a covalent inhibitor. In some embodiments, the EGFR-targeted therapeutic agent is osimertinib (AZD9291, merelectinib, TAGRISSO™), erlotinib (TARCEVA®), gefitinib (IRESSA®), cetuximab (ERBITUX®), necitumumab (PORTRAZZA™, IMC-11F8), neratinib (HKI-272, NERLYNX®), lapatinib (TYKERB®), panitumumab (ABX-EGF, VECTIBIX®), vandetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (AC0010), AC0010MA EAI045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyrylshikonin, acetylshikonin, β,β-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, R05083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is selected from osimertinib, gefitinib, erlotinib, afatinib, lapatinib, neratinib, AZD-9291, CL-387785, CO-1686, or WZ4002.

Additional EGFR-targeted therapeutic agents (e.g., a first EGFR inhibitor or a second EGFR inhibitor) include those disclosed in WO 2019/246541; WO 2019/165385; WO 2014/176475; and U.S. Pat. No. 9,029,502, each of which is incorporated by reference in its entirety.

In some embodiments, the other HER2-targeted therapeutic is a multi-kinase inhibitor exhibiting HER2 inhibition activity. In some embodiments, the other HER2-targeted therapeutic inhibitor is selective for a HER2 kinase.

Non-limiting examples of HER2-targeted therapeutic agents (e.g., a first HER2 inhibitor or a second HER2 inhibitor) include a HER2-selective inhibitor, a panHER inhibitor, and an anti-HER2 antibody. Exemplary HER2-targeted therapeutic agents include trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

Additional HER2-targeted therapeutic agents (e.g., a first HER2 inhibitor or a second HER2 inhibitor) include those disclosed in WO 2019/246541; WO 2019/165385; WO 2014/176475; and U.S. Pat. No. 9,029,502, each of which is incorporated by reference in its entirety.

A “RAS pathway targeted therapeutic agent” as used herein includes any compound exhibiting inactivation activity of any protein in a RAS pathway (e.g., kinase inhibition, allosteric inhibition, inhibition of dimerization, and induction of degradation). Non-limiting examples of a protein in a RAS pathway include any one of the proteins in the RAS-RAF-MAPK pathway or PI3K/AKT pathway such as RAS (e.g., KRAS, HRAS, and NRAS), RAF, BRAF, MEK, ERK, PI3K, AKT, and mTOR. In some embodiments, a RAS pathway modulator can be selective for a protein in a RAS pathway, e.g., the RAS pathway modulator can be selective for RAS (also referred to as a RAS modulator). In some embodiments, a RAS modulator is a covalent inhibitor. In some embodiments, a RAS pathway targeted therapeutic agent is a “KRAS pathway modulator.” A KRAS pathway modulator includes any compound exhibiting inactivation activity of any protein in a KRAS pathway (e.g., kinase inhibition, allosteric inhibition, inhibition of dimerization, and induction of degradation). Non-limiting examples of a protein in a KRAS pathway include any one of the proteins in the KRAS-RAF-MAPK pathway or PI3K/AKT pathway such as KRAS, RAF, BRAF, MEK, ERK, PI3K, AKT, and mTOR. In some embodiments, a KRAS pathway modulator can be selective for a protein in a RAS pathway, e.g., the KRAS pathway modulator can be selective for KRAS (also referred to as a KRAS modulator). In some embodiments, a KRAS modulator is a covalent inhibitor. Non-limiting examples of a KRAS-targeted therapeutic agents (e.g., KRAS inhibitors) include BI 1701963, AMG 510, ARS-3248, ARS1620, AZD4785, SML-8-73-1, SML-10-70-1, VSA9, AA12, and MRTX-849.

Further non-limiting examples of RAS-targeted therapeutic agents include BRAF inhibitors, MEK inhibitors, ERK inhibitors, PI3K inhibitors, AKT inhibitors, and mTOR inhibitors. In some embodiments, the BRAF inhibitor is vemurafenib (ZELBORAF®), dabrafenib (TAFINLAR®), and encorafenib (BRAFTOVI™), BMS-908662 (XL281), sorafenib, LGX818, PLX3603, RAF265, RO5185426, GSK2118436, ARQ 736, GDC-0879, PLX-4720, AZ304, PLX-8394, HM95573, RO5126766, LXH254, or a combination thereof.

In some embodiments, the MEK inhibitor is trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, RO5126766, CS3006, WX-554, PD98059, CI1040 (PD184352), hypothemycin, or a combination thereof.

In some embodiments, the ERK inhibitor is FRI-20 (ON-01060), VTX-11e, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, ONC201, or a combination thereof.

In some embodiments, PI3K inhibitor is selected from buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, or a combination thereof.

In some embodiments, the AKT inhibitor is selected from miltefosine (IMPADIVO®), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, miltefosine, perifosine, erucylphophocholine, erufosine, SR13668, OSU-A9, PH-316, PHT-427, PIT-1, DM-PIT-1, triciribine (Triciribine Phosphate Monohydrate), API-1, N-(4-(5-(3-acetamidophenyl)-2-(2-aminopyridin-3-yl)-3H-imidazo[4,5-b] pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, ONC201, or a combination thereof.

In some embodiments, the mTOR inhibitor is selected from MLN0128, AZD-2014, CC-223, AZD2014, CC-115, everolimus (RAD001), temsirolimus (CCI-779), ridaforolimus (AP-23573), sirolimus (rapamycin), or a combination thereof.

Non-limiting examples of farnesyl transferase inhibitors include lonafarnib, tipifarnib, BMS-214662, L778123, L744832, and FTI-277.

In some embodiments, a chemotherapeutic agent includes an anthracycline, cyclophosphamide, a taxane, a platinum-based agent, mitomycin, gemcitabine, eribulin (HALAVEN™), or combinations thereof.

Non-limiting examples of a taxane include paclitaxel, docetaxel, abraxane, and taxotere.

In some embodiments, the anthracycline is selected from daunorubicin, doxorubicin, epirubicin, idarubicin, and combinations thereof.

In some embodiments, the platinum-based agent is selected from carboplatin, cisplatin, oxaliplatin, nedaplatin, triplatin tetranitrate, phenanthriplatin, picoplatin, satraplatin and combinations thereof.

Non-limiting examples of PARP inhibitors include olaparib (LYNPARZA®), talazoparib, rucaparib, niraparib, veliparib, BGB-290 (pamiparib), CEP 9722, E7016, iniparib, IMP4297, NOV1401, 2X-121, ABT-767, RBN-2397, BMN 673, KU-0059436 (AZD2281), BSI-201, PF-01367338, INO-1001, and JPI-289.

Non-limiting examples of immunotherapy include immune checkpoint therapies, atezolizumab (TECENTRIQ®), albumin-bound paclitaxel. Non-limiting examples of immune checkpoint therapies include inhibitors that target CTLA-4, PD-1, PD-L1, BTLA, LAG-3, A2AR, TIM-3, B7-H3, VISTA, IDO, and combinations thereof. In some embodiments the CTLA-4 inhibitor is ipilimumab (YERVOY®). In some embodiments, the PD-1 inhibitor is selected from pembrolizumab (KEYTRUDA®), nivolumab (OPDIVO®), cemiplimab (LIBTAYO®), or combinations thereof. In some embodiments, the PD-L1 inhibitor is selected from atezolizumab (TECENTRIQ®), avelumab (BAVENCIO®), durvalumab (IMFINZI®), or combinations thereof. In some embodiments, the LAG-3 inhibitor is IMP701 (LAG525). In some embodiments, the A2AR inhibitor is CPI-444. In some embodiments, the TIM-3 inhibitor is MBG453. In some embodiments, the B7-H3 inhibitor is enoblituzumab. In some embodiments, the VISTA inhibitor is JNJ-61610588. In some embodiments, the IDO inhibitor is indoximod. See, for example, Marin-Acevedo, et al., J Hematol Oncol. 11: 39 (2018).

In some embodiments, the additional therapy or therapeutic agent is a combination of atezolizumab and nab-paclitaxel.

Accordingly, also provided herein is a method of treating cancer, comprising administering to a subject in need thereof a pharmaceutical combination for treating cancer which comprises (a) a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, (b) an additional therapeutic agent, and (c) optionally at least one pharmaceutically acceptable carrier for simultaneous, separate or sequential use for the treatment of cancer, wherein the amounts of the compound of Formula (I), or a pharmaceutically acceptable salt thereof, and the additional therapeutic agent are together effective in treating the cancer.

In some embodiments, the additional therapeutic agent(s) includes any one of the above listed therapies or therapeutic agents which are standards of care in cancers wherein the cancer has a dysregulation of an EGFR gene, an EGFR protein, or expression or activity, or level of any of the same.

In some embodiments, the additional therapeutic agent(s) includes any one of the above listed therapies or therapeutic agents which are standards of care in cancers wherein the cancer has a dysregulation of a HER2 gene, a HER2 kinase, or expression or activity, or level of any of the same.

These additional therapeutic agents may be administered with one or more doses of the compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art.

Also provided herein is (i) a pharmaceutical combination for treating a cancer in a subject in need thereof, which comprises (a) a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (I), or pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the cancer is an EGFR-associated cancer. For example, an EGFR-associated cancer having one or more EGFR inhibitor resistance mutations. In some embodiments, the cancer is a HER2-associated cancer. For example, a HER2-associated cancer having one or more HER2 inhibitor resistance mutations.

The term “pharmaceutical combination”, as used herein, refers to a pharmaceutical therapy resulting from the mixing or combining of more than one active ingredient and includes both fixed and non-fixed combinations of the active ingredients. The term “fixed combination” means that a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, and at least one additional therapeutic agent (e.g., a chemotherapeutic agent), are both administered to a subject simultaneously in the form of a single composition or dosage. The term “non-fixed combination” means that a compound of Formula (I), or a pharmaceutically acceptable salt thereof, and at least one additional therapeutic agent (e.g., chemotherapeutic agent) are formulated as separate compositions or dosages such that they may be administered to a subject in need thereof simultaneously, concurrently or sequentially with variable intervening time limits, wherein such administration provides effective levels of the two or more compounds in the body of the subject. These also apply to cocktail therapies, e.g., the administration of three or more active ingredients

Accordingly, also provided herein is a method of treating a cancer, comprising administering to a subject in need thereof a pharmaceutical combination for treating cancer which comprises (a) a compound of Formula (I) (e.g., Formula (I-a), (I-b), (I-c), (I-d), (I-e), (I-f), (I-g), (I-h), (I-i), (I-j), or (I-k)), or pharmaceutically acceptable salt thereof, and (b) an additional therapeutic agent, wherein the compound of Formula (I) and the additional therapeutic agent are administered simultaneously, separately or sequentially, wherein the amounts of the compound of Formula (I), or pharmaceutically acceptable salt thereof, and the additional therapeutic agent are together effective in treating the cancer. In some embodiments, the compound of Formula (I), or pharmaceutically acceptable salt thereof, and the additional therapeutic agent are administered simultaneously as separate dosages. In some embodiments, the compound of Formula (I), or pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I), or pharmaceutically acceptable salt thereof, and the additional therapeutic agent are administered simultaneously as a combined dosage. In some embodiments, the cancer is an EGFR-associated cancer. For example, an EGFR-associated cancer having one or more EGFR inhibitor resistance mutations. In some embodiments, the cancer is a HER2-associated cancer. For example, a HER2-associated cancer having one or more HER2 inhibitor resistance mutations.

In some embodiments, the presence of one or more EGFR inhibitor resistance mutations in a tumor causes the tumor to be more resistant to treatment with a first EGFR inhibitor. Methods useful when an EGFR inhibitor resistance mutation causes the tumor to be more resistant to treatment with a first EGFR inhibitor are described below. For example, provided herein are methods of treating a subject having a cancer that include: identifying a subject having a cancer cell that has one or more EGFR inhibitor resistance mutations; and administering to the identified subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. In some embodiments, the compound of Formula (I), or a pharmaceutically acceptable salt thereof, is administered in combination with the first EGFR inhibitor. Also provided are methods of treating a subject identified as having a cancer cell that has one or more EGFR inhibitor resistance mutations that include administering to the subject a compound of Formula (I), or a pharmaceutically acceptable salt thereof. In some embodiments, the compound of Formula (I), or a pharmaceutically acceptable salt thereof, is administered in combination with the first EGFR inhibitor. In some embodiments, the one or more EGFR inhibitor resistance mutations confer increased resistance to a cancer cell or tumor to treatment with the first EGFR inhibitor. In some embodiments, the one or more EGFR inhibitor resistance mutations include one or more EGFR inhibitor resistance mutations listed in Table 2a and 2b. For example, the one or more EGFR inhibitor resistance mutations can include a substitution at amino acid position 718, 747, 761, 790, 797, or 854 (e.g., L718Q, L747S, D761Y, T790M, C797S, and T854A).

For example, provided herein are methods for treating an EGFR-associated cancer in a subject in need of such treatment, the method comprising (a) detecting a dysregulation of an EGFR gene, an EGFR kinase, or the expression or activity or level of any of the same in a sample from the subject; and (b) administering to the subject a therapeutically effective amount of a first EGFR inhibitor, wherein the first EGFR inhibitor is selected from the group consisting of osimertinib, gefitinib, erlotinib, afatinib, lapatinib, neratinib, AZD-9291, CL-387785, CO-1686, or WZ4002. In some embodiments, the methods further comprise (after (b)) (c) determining whether a cancer cell in a sample obtained from the subject has at least one EGFR inhibitor resistance mutation; and (d) administering a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof, as a monotherapy or in conjunction with another anticancer agent to the subject if the subject has been determined to have a cancer cell that has at least one EGFR inhibitor resistance mutation; or (e) administering additional doses of the first EGFR inhibitor of step (b) to the subject if the subject has not been determined to have a cancer cell that has at least one EGFR inhibitor resistance mutation.

Methods useful when a HER2 activating mutation is present in a tumor are described herein. For example, provided herein are methods of treating a subject having a cancer that include: identifying a subject having a cancer cell that has one or more HER2 activating mutations; and administering to the identified subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof. Also provided are methods of treating a subject identified as having a cancer that has one or more HER2 activating mutations that include administering to the subject a compound of Formula (I), or a pharmaceutically acceptable salt thereof. In some embodiments, the one or more HER2 activating mutations include one or more HER2 activating mutations listed in Tables 3-5.

Methods useful when an activating mutation (e.g., HER2 activating mutation) is present in a tumor in a subject are described herein. For example, provided herein are methods of treating a subject having a cancer that include: identifying a subject having a cancer cell that has one or more HER2 activating mutations; and administering to the identified subject a compound of Formula (I) (e.g., Formula (I-a), (I-b), (I-c), (I-d), (I-e), (I-f), (I-g), (I-h), (I-i), (I-j), or (I-k)), or a pharmaceutically acceptable salt thereof.

Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following Scheme 1, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry. Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### Example 1: Synthesis of 2-(2-aminopyrimidin-4-yl)-3-((3-chloro-2-methoxyphenyl)amino)-1,5,6,7-tetrahydro-4H-pyrrolo[3,2-c]pyridin-4-one (Compound 148)

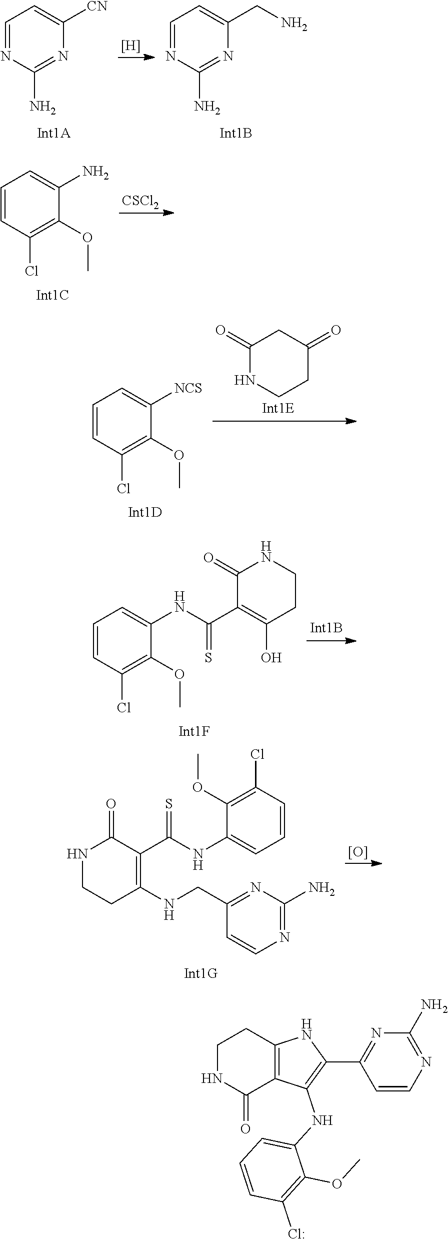

Cyanopyrimidine Int1A is hydrogenated in the presence of hydrogen gas and a catalyst, e.g, Raney Ni in a polar protic solvent e.g., MeOH to give Int1B. Int1C is reacted with thiophosgene under modified Schotten-Baumann conditions, e.g., NaHCO₃ in the presence of water/DCM to give the corresponding thioisocyanate Int1D. Treatment of Int1D with Int1E in the presence of a strong base, e.g., DBU in a polar aprotic solvent, e.g., ACN gives Int1F. Condensation of Int1F with Int1B with heating, e.g., at 120° C. in the presence of a dehydrating agent, e.g., 4A molecular sieves in a polar aprotic solvent, e.g., DMA provides Int1G. Oxidative cyclization in the presence of a mild oxidant, e.g., H₂O₂ in and polar protic solvent, e.g., MeOH with heating, e.g., 50° C. gives the title compound.

### Example 2: Synthesis of 3-((3-chloro-2-methoxyphenyl)amino)-2-(6-methoxy-1,5-naphthyridin-4-yl)-1,5,6,7-tetrahydro-4H-pyrrolo[3,2-c]pyridin-4-one (Compound 149)

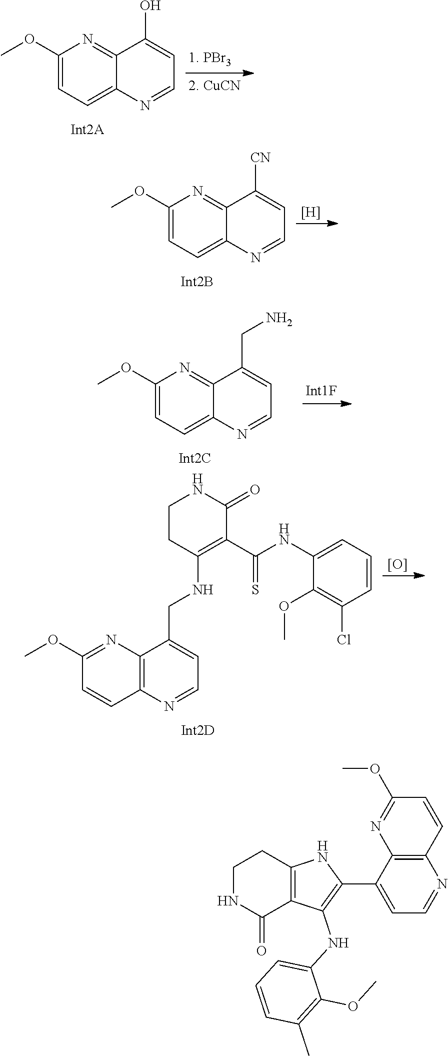

Int2A is treated with a brominating agent, e.g., PBr₃ in a polar aprotic solvent, e.g., DMF to afford the corresponding bromide, which is converted to the corresponding cyano adduct Int2B by heating e.g., at 130° C. under copper catalyzed cyanation conditions, e.g., with CuCN in a polar aprotic solvent, e.g., DMF. Hydrogenation of Int2B in the presence of hydrogen gas and a catalyst, e.g, Raney Ni in a polar protic solvent e.g., MeOH with acetic acid gives Int2C. Condensation of Int2C with Int1F (from Example 1) with heating, e.g., 120° C. in the presence of a dehydrating agent, e.g., 4A molecular sieves in a polar aprotic solvent, e.g., DMA provides Int2D. Oxidative cyclization in the presence of TFA, a mild oxidant, e.g., H₂O₂ in polar protic solvent, e.g., MeOH with heating, e.g., 40° C. gives the title compound.

### Example 3: Synthesis of 3-((3-fluoro-2-methoxyphenyl)amino)-2-(7-methyl-7H-pyrrolo[2,3-d]pyrimidin-4-yl)-1,5,6,7-tetrahydro-4H-pyrrolo[3,2-c]pyridin-4-one (Compound 150)

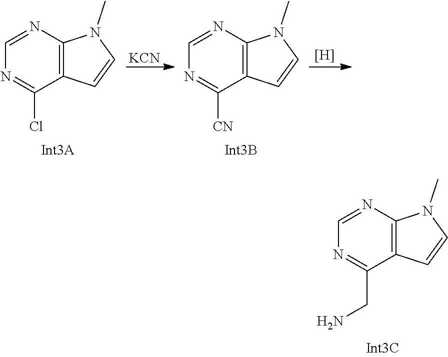

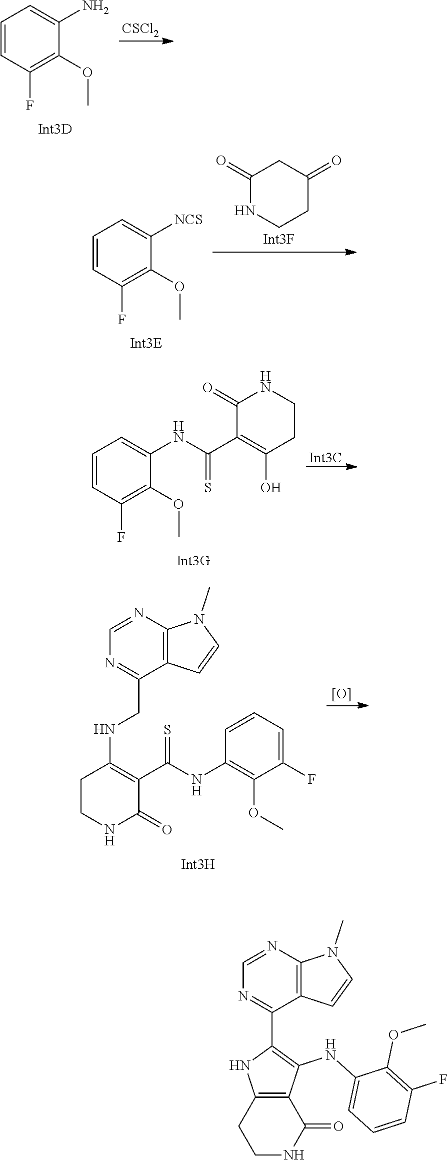

Int3A is converted to the corresponding cyano adduct Int3B by heating e.g., 130° C. with a cyanide source, e.g., KCN in a polar aprotic solvent, e.g., DMSO in the presence of SPTS. Int3B is hydrogenated in the presence of hydrogen gas and a catalyst, e.g, Pd/C in a polar protic solvent e.g., MeOH to give Int3C. Int3D is reacted with thiophosgene under modified Schotten-Baumann conditions, e.g., NaHCO₃ in the presence of water/DCM to give the corresponding thioisocyanate Int3E. Treatment of Int3E with Int3F in the presence of a strong base, e.g. DBU in a polar aprotic solvent, e.g., ACN gives Int3G. Condensation of Int3G with Int3C under amide coupling conditions, e.g., PyBOP, DIEA in a polar aprotic solvent, e.g., DMF gives Int3H. Oxidative cyclization of Int3H in the presence of TFA, a mild oxidant, e.g., H₂O₂ in polar protic solvent, e.g., MeOH at room temperature gives the title compound.

### Example 4: Synthesis of 3-((3-fluoro-2-methoxyphenyl)amino)-2-(thieno[2,3-d]pyrimidin-4-yl)-1,5,6,7-tetrahydro-4H-pyrrolo[3,2-c]pyridin-4-one (Compound 151)

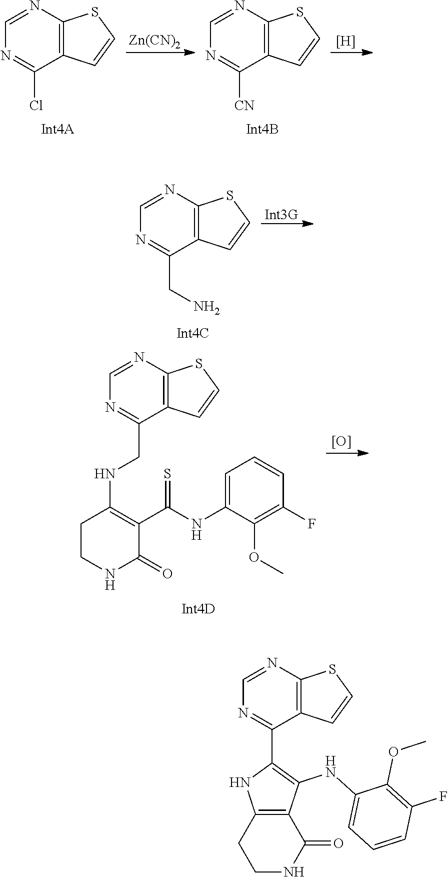

Int4A is converted to the corresponding cyano adduct Int4B by Pd-catalyzed cyanation, e.g., Zn(CN)₂, Zn, Pd₂(dba)₃, Pd(dppf)Cl₂ in a polar aprotic solvent, e.g., DMA. Hydrogenation of Int4B with a catalyst, e.g., Raney Ni and hydrogen gas in a polar protic solvent e.g., MeOH gives Int4C. Condensation of Int4C with Int3G (from Example 3) under amide coupling conditions, e.g., PyBOP, DIEA in a polar aprotic solvent, e.g., DMF gives at 0° C. gives Int4D. Oxidative cyclization in the presence of a mild oxidant, e.g., H₂O₂ in polar solvent, e.g., DMSO with heating, e.g., 100° C. gives the title compound.

### Example 5: Synthesis of 3-((3-chloro-2-methoxyphenyl)amino)-2-(6-(methylamino)-1,5-naphthyridin-4-yl)-1,5,6,7-tetrahydro-4H-pyrrolo[3,2-c]pyridin-4-one (Compound 152)

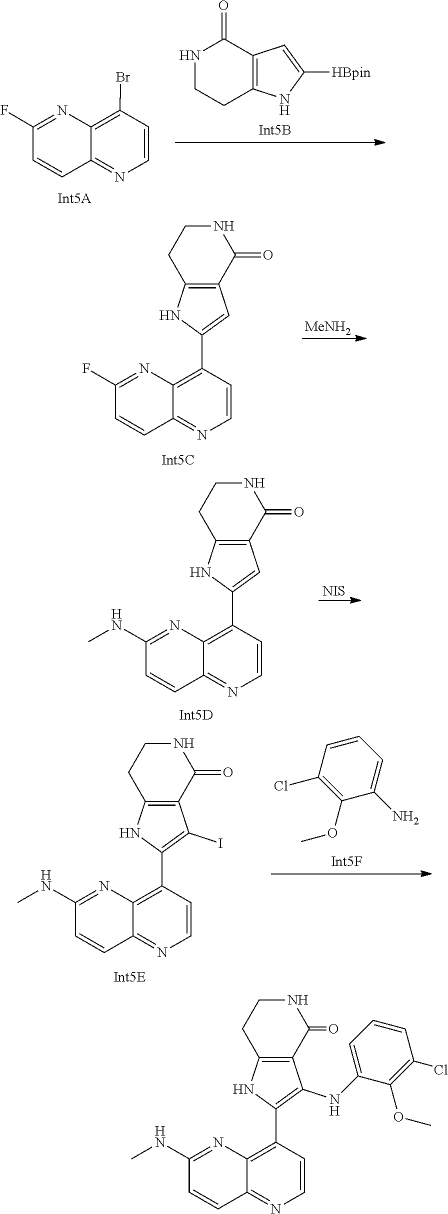

Int5A is treated with Int5B under Suzuki cross-coupling conditions, e.g., Pd(dppf)Cl₂, with a mild base, e.g., TEA in a polar solvent mixture, e.g., DME/water to give Int5C. Displacement of the fluoride of Int5C with methylamine gives Int5D, which is then iodinated with NIS in a polar aprotic solvent, e.g., DMF to give Int5E. Coupling of Int5E with of Int5F under Buchwald conditions, e.g., Ephos, Ephos Pd G4 in the presence of a base, e.g., Cs₂CO₃ in a polar aprotic solvent e.g., 1,4-dioxane at elevated temperature, e.g., 100° C. gives the title compound.

### Example 6: Synthesis of 3-((3-fluoro-2-methoxyphenyl)amino)-2-(6-methoxyimidazo[1,2-a]pyridin-3-yl)-1,5,6,7-tetrahydro-4H-pyrrolo[3,2-c]pyridin-4-one (Compound 153)

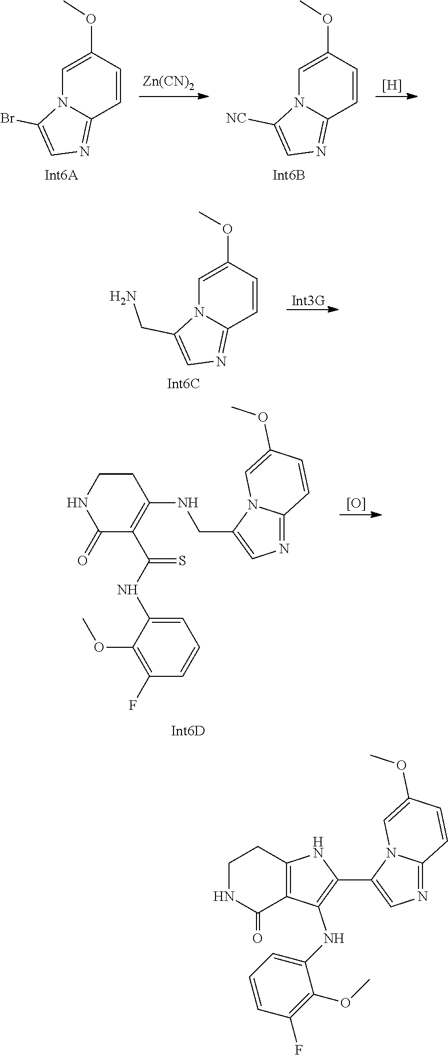

Int6A is converted to the corresponding cyano adduct Int6B by Pd-catalyzed cyanation, e.g., Zn(CN)₂, Pd(PPh₃)₄ in a polar aprotic solvent, e.g., DMA at an elevated temperature, e.g., at 120° C. Reduction of Int6B with a strong reducing agent e.g., LAH in a polar aprotic solvent, e.g., THF at room temperature gives Int6C. Condensation of Int6C with Int3G (from Example 3) with heating, e.g., 120° C. in the presence of a dehydrating agent, e.g., 4A molecular sieves in a polar aprotic solvent, e.g., DMA provides Int6D. Oxidative cyclization in the presence of a mild oxidant, e.g., H₂O₂ in a polar protic solvent, e.g., MeOH with heating, e.g., 50° C. gives the title compound.

### Example 7: Synthesis of 3-((3-fluoro-2-methoxyphenyl)amino)-2-(pyrido[3,2-d]pyrimidin-4-yl)-1,5,6,7-tetrahydro-4H-pyrrolo[3,2-c]pyridin-4-one (Compound 154)

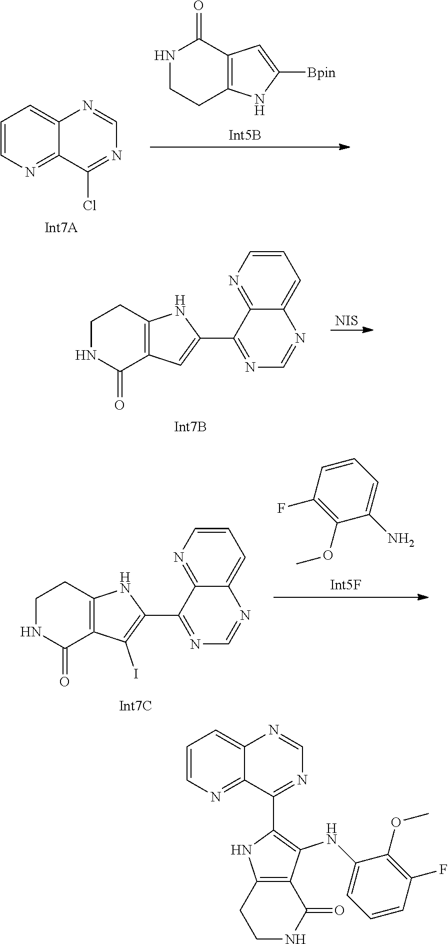

Int7A is treated with Int5B under Suzuki cross-coupling conditions, e.g., Pd(PPh₃)₄, K₂CO₃, in a polar solvent mixture, e.g., dioxane/water to give Int7B, which is iodinated with NIS in a polar aprotic solvent, e.g., DMF to give Int7C. Coupling of Int7C with of Int5F under Buchwald conditions, e.g., Ephos, Ephos Pd G4 in the presence of a base, e.g., Cs₂CO₃ in 1,4-dioxane at elevated temperature, e.g., 50° C. gives the title compound.

### Example 8: Synthesis of 4-(3-((3-fluoro-2-methoxyphenyl)amino)-4-oxo-4,5,6,7-tetrahydro-1H-pyrrolo[3,2-c]pyridin-2-yl)-2-methylisoquinolin-1 (2H)-one (Compound 155)

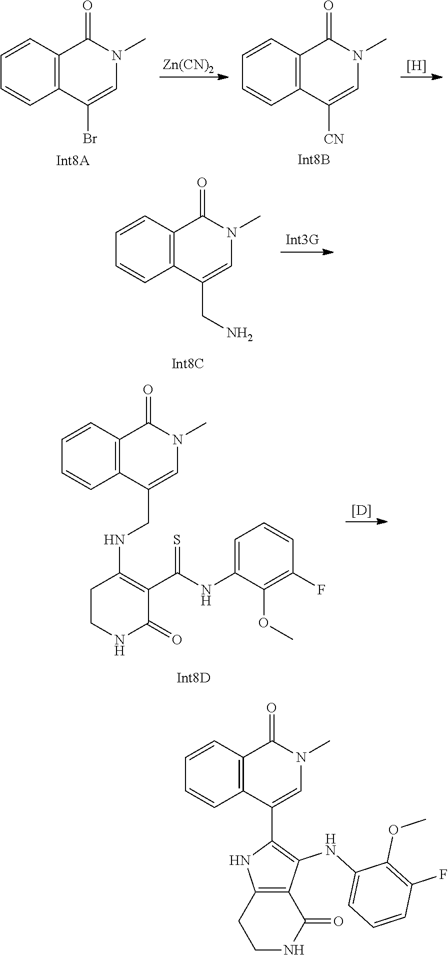

Int8A is converted to the corresponding cyano adduct Int8B by Pd-catalyzed cyanation, e.g., Zn(CN)₂, Pd(PPh₃)₄, in a polar aprotic solvent, e.g., DMF at an elevated temperature, e.g., at 120° C. Reduction of Int8B with a catalyst, e.g., Raney Ni in the presence of hydrogen (e.g., 5 atm) in NH₃/MeOH at room temperature gives Int8C. Condensation of Int8C with Int3G (from Example 3) under amide coupling conditions, e.g., PyBOP, DIEA in a polar aprotic solvent, e.g., DMF at 0° C. gives Int8D. Oxidative cyclization of Int8D in the presence of a mild oxidant, e.g., H₂O₂ in a protic solvent gives the title compound.

### Example 9: Synthesis of 3-((3-fluoro-2-methoxyphenyl)amino)-2-(isothiazolo[4,3-b]pyridin-3-yl)-1,5,6,7-tetrahydro-4H-pyrrolo[3,2-c]pyridin-4-one (Compound 156)

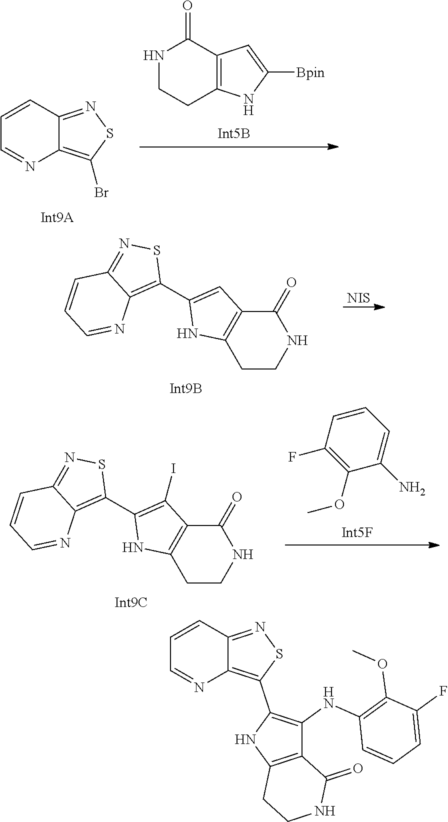

Int9A is treated with Int5B under Suzuki cross-coupling conditions, e.g., Pd(dppf)Cl₂ in the presence of a mild base, e.g, K₂CO₃ in a polar solvent mixture, e.g., 1,4-dioxane/water to give Int9B, which is iodinated with NIS in a polar aprotic solvent, e.g., DMF to give Int9C. Coupling of Int9C with of Int5F under Buchwald conditions, e.g., E-phos Pd G4 in the presence of a strong base, e.g., LiHMDS in 1,4-dioxane at elevated temperature, e.g., 100° C. gives the title compound.

### Example 10: Synthesis of 2-(benzo[c]isothiazol-3-yl)-3-((3-fluoro-2-methoxyphenyl)amino)-1,5,6,7-tetrahydro-4H-pyrrolo[3,2-c]pyridin-4-one (Compound 157)

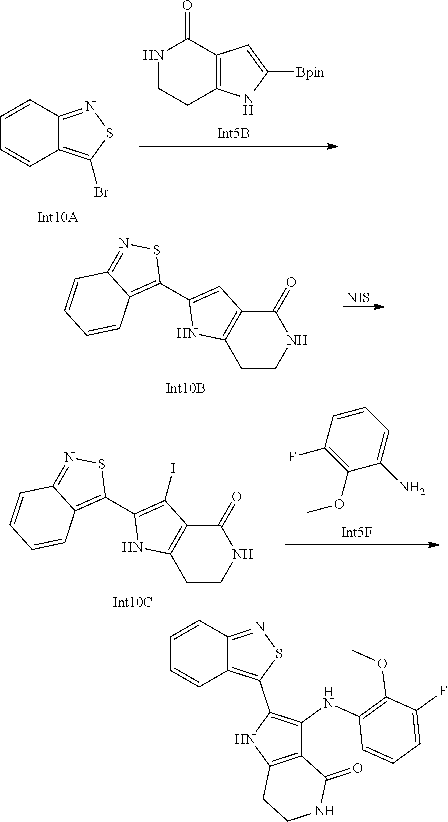

Int10A is treated with Int5B under Suzuki cross-coupling conditions, e.g., Pd(dppf)Cl₂ in the presence of a mild base, e.g, K₂CO₃ in a polar solvent mixture, e.g., 1,4-dioxane/water to give Int10B, which is iodinated with NIS in a polar aprotic solvent, e.g., DMF to give Int10C. Coupling of Int10C with of Int5F under Buchwald conditions, e.g., E-phos Pd G4 in the presence of a base, e.g., Cs₂CO₃ in polar aprotic solvent, e.g., 1,4-dioxane at elevated temperature, e.g., 100° C. gives the title compound.

### Example 11: Synthesis of 3-((3-fluoro-2-methoxyphenyl)amino)-2-(1H-pyrazolo[3,4-d]pyrimidin-4-yl)-1,5,6,7-tetrahydro-4H-pyrrolo[3,2-c]pyridin-4-one (Compound 158)

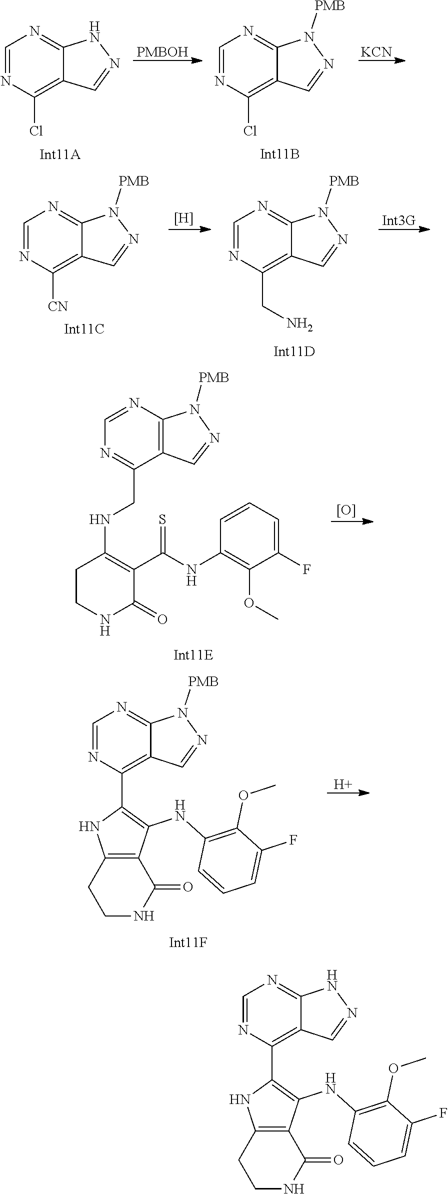

Int11A is PMB-protected by treatment with p-methoxybenzyl alcohol under Mitsunobu conditions, e.g. PPh₃/DIAD in a polar aprotic solvent (e.g., THF) to give Int11B, which is cyanated via SNAr with cyanide source, e.g., KCN in a polar aprotic solvent, e.g., DMF at elevated temperature (e.g., 60° C.). Hydrogenation of Int11C in the presence of catalytic Pd, e.g., Pd/C under a hydrogen atmosphere in a polar protic solvent, e.g., MeOH gives Int11D. Condensation of Int11D with Int3G (from Example 3) under amide coupling conditions, e.g., PyBOP, DIEA in a polar aprotic solvent, e.g., DMF gives at room temperature gives Int11E. Oxidative cyclization of Int11E in the presence of a mild oxidant, e.g., H₂O₂ in a polar aprotic solvent, e.g., DMSO with heating, e.g., 100° C. gives Int11F, which is deprotected with strong acid, e.g., TFA to give the title compound.

### Example 12: Synthesis of 3-((3-fluoro-2-methoxyphenyl)amino)-2-(2-phenylpyridin-4-yl)-1,5,6,7-tetrahydro-4H-pyrrolo[3,2-c]pyridin-4-one (Compound 159)

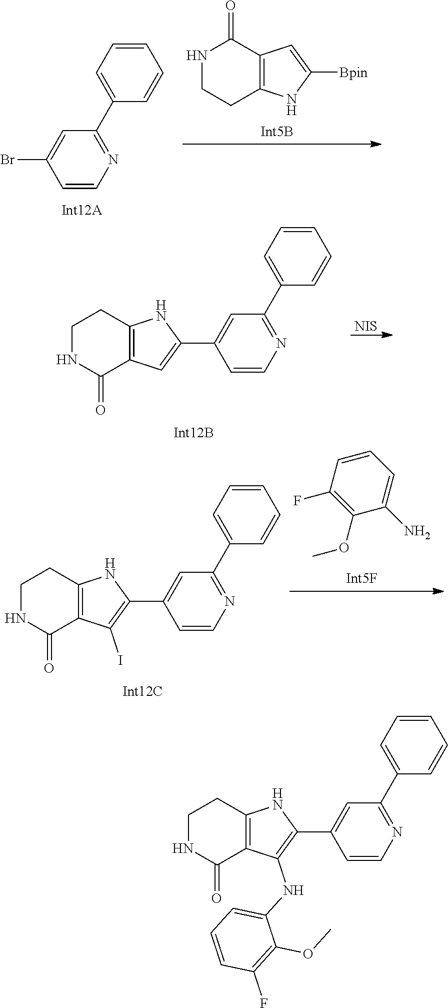

Int12A is treated with Int5B under Suzuki cross-coupling conditions, e.g., Pd(dppf)Cl₂ in the presence of a mild base, e.g, K₂CO₃ in a polar solvent mixture, e.g., 1,4-dioxane/water to give Int12B, which is iodinated with NIS in a polar aprotic solvent, e.g., DMF to give Int12C. Coupling of Int12C with of Int5F under Buchwald conditions, e.g., E-phos Pd G4 in the presence of a base, e.g., Cs₂CO₃ in a polar aprotic solvent, e.g., 1,4-dioxane at elevated temperature, e.g., 100° C. gives the title compound.

### Example 13: Synthesis of 1-acryloyl-3′-((3-fluoro-2-methoxyphenyl)amino)-2′-(6-methoxy-1,5-naphthyridin-4-yl)-5′,6′-dihydrospiro[piperidine-4,7′-pyrrolo[3,2-c]pyridin]-4′(1′H)-one (Compound 160)

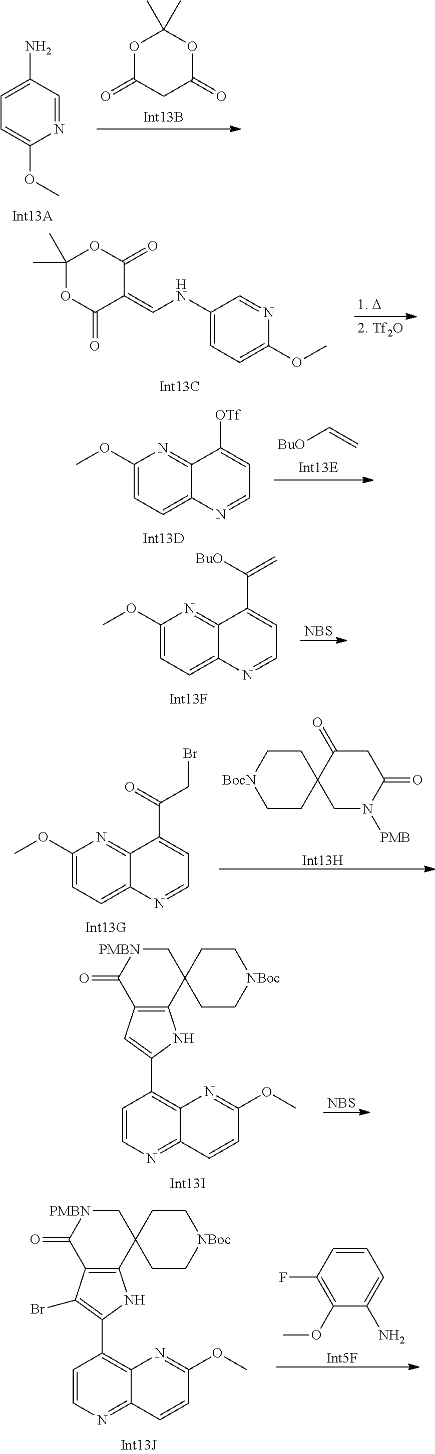

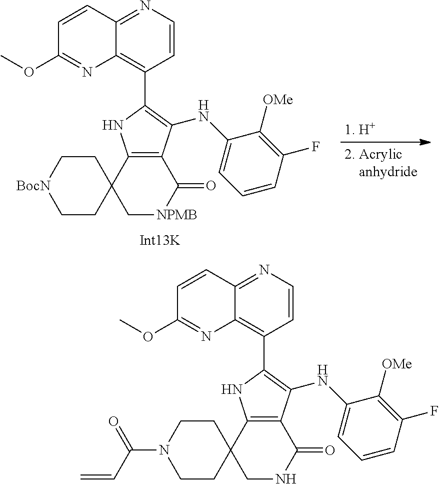

Commercially available Int13A is condensed with Meldrum's acid Int13B in the presence of an orthoformate, e.g., triethylorthoformate in a polar protic solvent, e.g., EtOH at an elevated temperature, e.g., 78° C. to give Int13C. Cyclization of Int13C at an elevated temperature in a high boiling solvent, e.g., Dowtherm at 220° C. followed by treatment of the resulting phenol with triflic anhydride gives Int13D. Coupling of Int13D with Int13E under Heck conditions, e.g., Pd(OAc)₂ and dppp in the presence of a mild base, e.g., TEA with heating, e.g., at 80° C. in a polar aprotic solvent (e.g., DMF, DMA) gives Int13F. Bromination of Int33F with a brominating agent, e.g., NBS in a halogenated solvent, e.g., DCM at a reduced temperature, e.g., 0° C. affords Int13G. Tandem alkylation/cyclization to give pyrrole Int13I occurs by treatment of Int13G with Int13 in the presence of NH₄OAc in a polar protic solvent, e.g., EtOH at room temperature. Bromination of Int13I with a brominating agent, e.g., NBS in a halogenated solvent, e.g., DCM affords Int13J, which is coupled with Int5F under Buchwald conditions, e.g., Pd₃(dba)₂, Xphos in the presence of base, e.g., Cs₂CO₃ in a high boiling point aprotic solvent, e.g., Toluene at an elevated temperature, e.g., 110° C. affords Int13K. Deprotection of Int13K with a strong acid, e.g., TFA or HCl followed by treatment with acrylic anhydride gives the title compound.

### Example 14: Synthesis of 1-acryloyl-2′-(2-aminopyrimidin-4-yl)-3′-((3-fluoro-2-methoxyphenyl)amino)-5′,6′-dihydrospiro[piperidine-4,7′-pyrrolo[3,2-c]pyridin]-4′(1′H)-one (Compound 162)

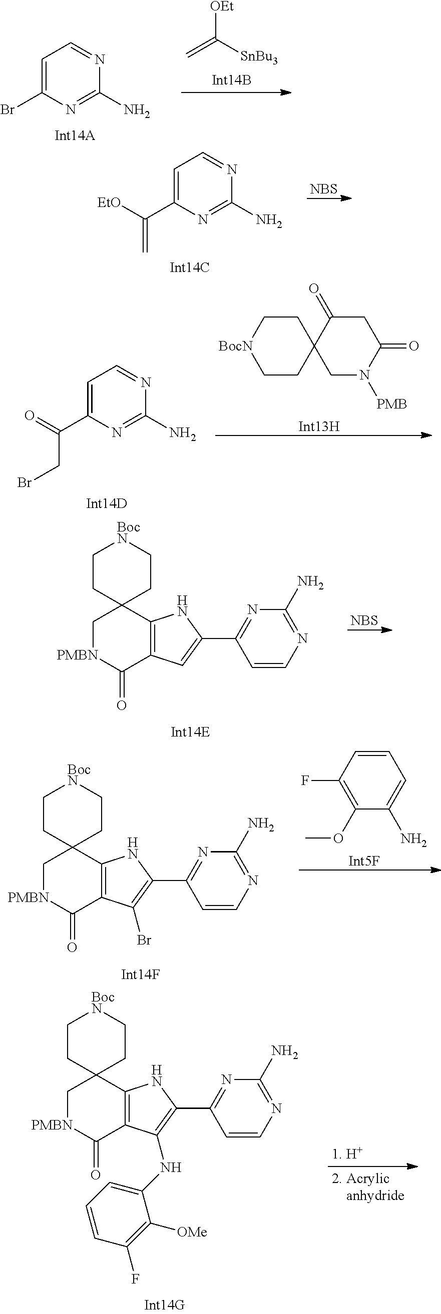

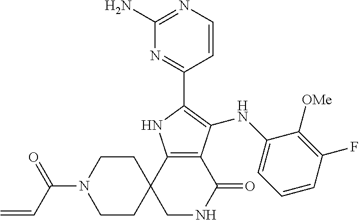 [SMILES: C=CC(=O)N1CCC2(CC1)CNC(=O)c1c2[nH]c(-c2ccnc(N)n2)c1Nc1cccc(F)c1OC]

Commercially available Int14A is coupled with commercially available Int14B under Stille conditions, e.g., Pd(dppf)Cl₂ in a polar aprotic solvent, e.g., DMF with mild heating to give Int14C. Treatment of Int14C with a brominating agent, e.g., NBS in a polar protic solvent mixture, e.g., THF-water affords Int14D. Tandem alkylation/cyclization to give pyrrole Int14E occurs by treatment of Int14D with Int13H in the presence of NH₄OAc in a polar protic solvent, e.g., EtOH at room temperature. Treatment of Int14E with a brominating agent, e.g., NBS in a halogenated solvent, e.g., DCM at reduced temperature (e.g., 0° C.) affords Int14F, which is coupled with Int5F under Buchwald conditions, e.g., Pd₃(dba)₂, Xphos in the presence of base, e.g., Cs₂CO₃ in a high boiling point aprotic solvent, e.g., Toluene at an elevated temperature, e.g., 110° C. affords Int14G. Deprotection of Int14G with a strong acid, e.g., TFA or HCl followed by treatment with acrylic anhydride gives the title compound.

### Example 15: Synthesis of 1-acryloyl-2′-(2-aminopyrimidin-4-yl)-3′-((3-fluoro-2-methoxyphenyl)amino)-5′,6′-dihydrospiro[azetidine-3,7′-pyrrolo[3,2-c]pyridin]-4′(1′H)-one (Compound 163)

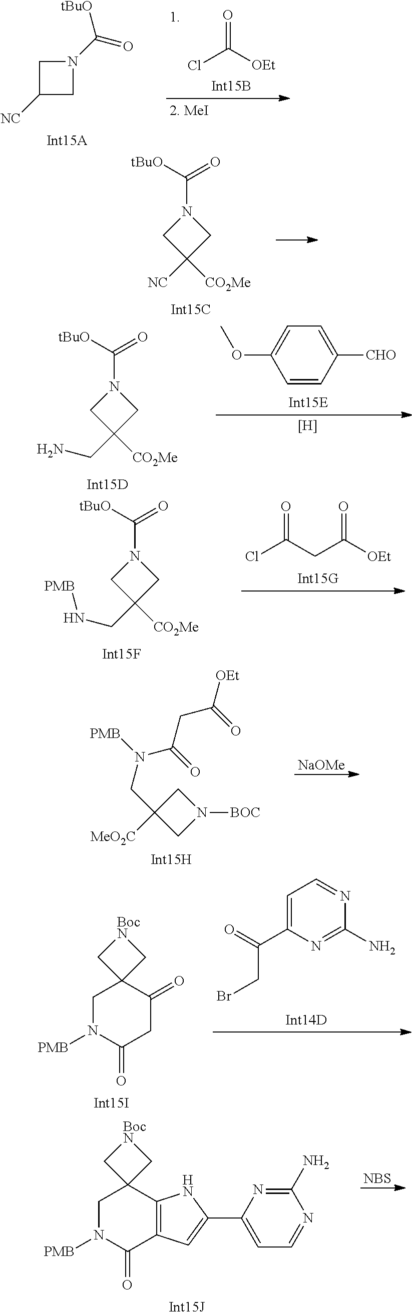

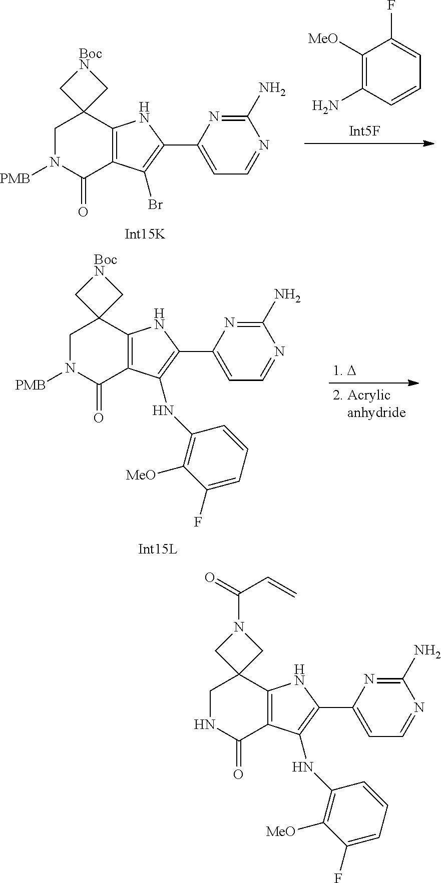

Reaction of Int15A and Int15B in the presence of a strong base, e.g., LiHMDS in a polar aprotic solvent, e.g., THF at reduced temperature (e.g., −78° C.), followed by methylation with MeI in the presence of a mild base, e.g., Cs₂CO₃ in a polar aprotic solvent (e.g., DMF) at reduced temperature, e.g., 0° C.-rt gives Int15C. Hydrogenation of Int15C in the presence of a catalyst, e.g., Pearlman's catalysts Pd(OH)₂ under a hydrogen atmosphere in a polar protic solvent, e.g., EtOH at room temperature gives Int15D. Protection of the amine with a PMB group occurs via treatment of Int15D with Int15E (4-methoxybenzaldehyde) followed by a mild reducing agent, e.g., NaCNBH₃ in a polar protic solvent, e.g., EtOH at elevated temperature (e.g., 45° C.) over several hours to give Int15F. Treatment of Int15F with Int15G under standard acylation conditions, e.g., pyridine, DMAP in a polar aprotic solvent, e.g., DCM at reduced temperature e.g., 0° C. gives Int15H. Dieckmann cyclization of Int15H with NaOMe in methanol at elevated temperature, e.g., 60° C. over several hours, followed by decarboxylation gives Int15I. Tandem alkylation/cyclization to give pyrrole Int15J occurs by treatment of Int15I with Int14D in the presence of NH₄OAc in a polar protic solvent, e.g., EtOH at room temperature. Treatment of Int15J with a brominating agent, e.g., NBS in a halogenated solvent, e.g., DCM at reduced temperature (e.g., 0° C.) affords Int15K, which is coupled with Int5F under Buchwald conditions, e.g., Pd₃(dba)₂, Xphos in the presence of base, e.g., Cs₂CO₃ in a high boiling point aprotic solvent, e.g., toluene at an elevated temperature, e.g., 110° C. to afford Int15L. Deprotection of Int15L via thermolysis of the BOC and PMB protecting groups by elevated heating (e.g., 140° C.) in a microwave in a high boiling point solvent, e.g., 1,3-xylene followed by treatment with acrylic anhydride under modified Schotten-Baumann conditions, e.g., TEA in the presence of water/THF at reduced temperature (e.g., 0° C.) gives the title compound.

### Example 16. (S)-3-((3-chloro-2-methoxyphenyl)amino)-7-(2-fluoroethyl)-2-(6-methoxy-1,5-naphthyridin-4-yl)-1,5,6,7-tetrahydro-4H-pyrrolo[3,2-c]pyridin-4-one (compound 744)

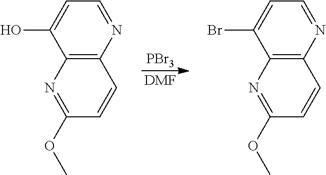

To a stirred mixture of 6-methoxy-1,5-naphthyridin-4-ol (2.0 g, 11.4 mmol, 1.0 equiv) in DMF (39.0 mL, 504 mmol, 44.4 equiv) was added PBr3 (1.1 mL, 11.6 mmol, 1.0 equiv) in portions at 0 degrees C. under nitrogen atmosphere. The resulting mixture was stirred for additional 1 h at 45 degrees C. The resulting mixture was cooled to rt. The precipitation was filtered out and washed with ether (100 mL). The filtrate cake was added water (50 mL) and NaOH (100 mL, 1 M), then extracted with DCM (3×100 mL). The combined organic layers were washed with brine (2×70 mL), dried over anhydrous Na₂SO₄. After filtration, the filtrate was concentrated under reduced pressure. The residue was purified by silica gel column chromatography, eluted with EtO/AcPE (5% to 60%) to afford 8-bromo-2-methoxy-1,5-naphthyridine (1.5 g, 46.0%) as a yellow solid.

LC-MS: M+H found: 239.0.

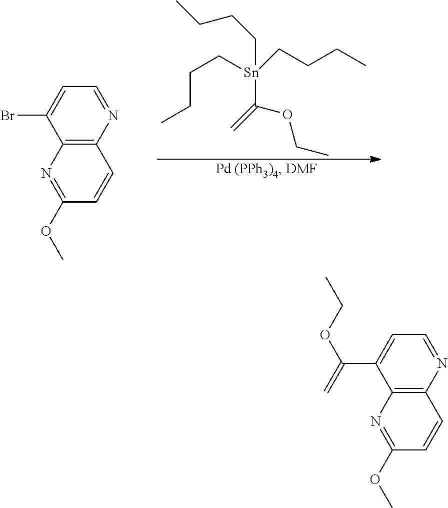

Into a 250-mL round-bottom flask purged and maintained with an inert atmosphere of nitrogen, were placed 8-bromo-2-methoxy-1,5-naphthyridine (5.0 g, 20.9 mmol, 1.0 equiv), DMF (60 mL), tributyl(1-ethoxyethenyl)stannane (9.06 g, 25.1 mmol, 1.2 equiv) and Pd(PPh₃)₄ (2.42 g, 2.09 mmol, 0.10 equiv) at ambient temperature. The resulting mixture was stirred for 16 h at 100 degrees C. The reaction progress was monitored by LCMS. The reaction was cooled to it and then diluted by the addition of EA (300 mL). The resulting solution was washed with brine (3×200 mL) and the organic layers were combined, dried over anhydrous sodium sulfate, filtrated and the filtrate was concentrated in vacuo to dryness. The residue was applied onto a silica gel column chromatography with ethyl acetate/petroleum ether (0-8%). This resulted in 8-(1-ethoxyethenyl)-2-methoxy-1,5-naphthyridine (4.3 g, 93.9%) as a yellow solid.

LC-MS: (M+H)⁺ found: 231.1.

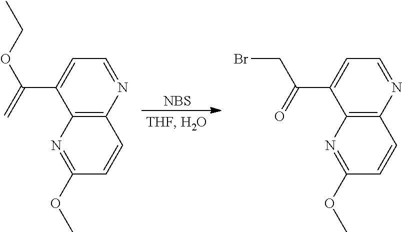

Into a 8-mL vial, were placed 8-(1-ethoxyethenyl)-2-methoxy-1,5-naphthyridine (1.0 g, 4.34 mmol, 1.0 equiv), THF (15 mL), water (1.5 mL) and NBS (0.77 g, 4.34 mmol, 1.0 equiv) at ambient temperature. The resulting mixture was stirred for 20 mins at rt. The reaction progress was monitored by LCMS. The reaction was then diluted by the addition of DCM (80 mL). The resulting solution was washed with the solution of NaHCO₃ in water (pH=8) (3×50 mL) and the organic layers were combined, dried over anhydrous sodium sulfate, filtrated and the filtrate was concentrated in vacuo to dryness. This resulted in (1.0 g, crude) as a brown solid.

LC-MS: (M+H)⁺ found: 281.0.

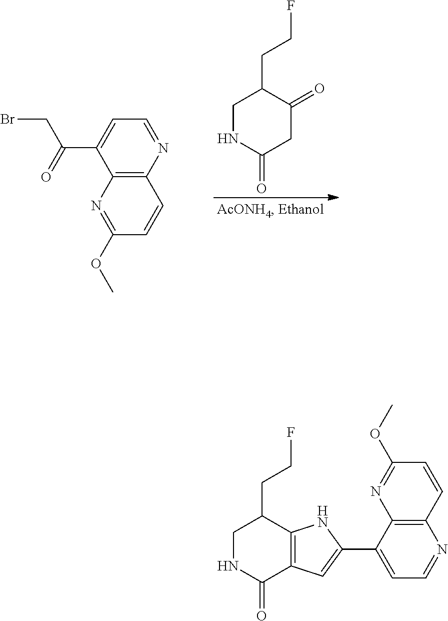 [SMILES: COc1ccc2nccc(C(=O)CBr)c2n1 ; O=C1CC(=O)C(CCF)CN1 ; COc1ccc2nccc(-c3cc4c([nH]3)C(CCF)CNC4=O)c2n1]

Into a 250-mL round-bottom flask, were placed tert-butyl 5-(2-fluoroethyl)-2,4-dioxopiperidine-1-carboxylate (1.0 g, 6.28 mmol, 1.0 equiv), EtOH (40 mL), 2-bromo-1-(6-methoxy-1,5-naphthyridin-4-yl)ethanone (1.77 g, 6.30 mmol, 1.0 equiv) and NH₄OAc (4.84 g, 62.8 mmol, 10.0 equiv) at ambient temperature. The resulting mixture was stirred for 2 h at rt and then 16 h at 50 degrees C. The reaction progress was monitored by LCMS. LCM showed 62% desired product. The reaction was then diluted by the addition of EA (300 mL). The resulting solution was extracted with brine (2×80 mL) and the organic layers were combined, dried over anhydrous sodium sulfate, filtrated and the filtrate was concentrated in vacuo to dryness. The residue was applied onto a silica gel column chromatography with dichloromethane/methanol (10/1). This resulted in 7-(2-fluoroethyl)-2-(6-methoxy-1,5-naphthyridin-4-yl)-1H,5H,6H,7H-pyrrolo[3,2-c]pyridin-4-one (390 mg, 18.2%) as a light yellow solid.

LC-MS: (M+H)⁺ found: 341.1.

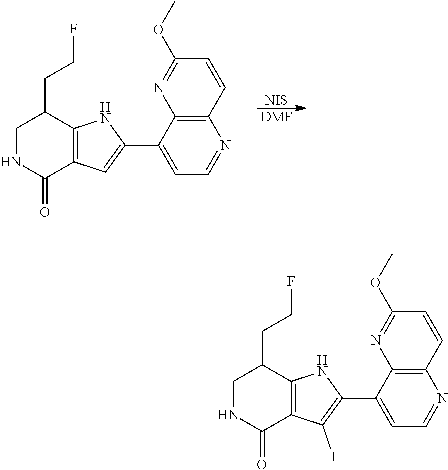

Into a 50-mL round-bottom flask, were placed 7-(2-fluoroethyl)-2-(6-methoxy-1,5-naphthyridin-4-yl)-1H,5H,6H,7H-pyrrolo[3,2-c]pyridin-4-one (370 mg, 1.09 mmol, 1.0 equiv), DMF (10 mL) and NIS (318 mg, 1.41 mmol, 1.30 equiv) at ambient temperature. The resulting mixture was stirred for 16 h at it. The reaction progress was monitored by LCMS. LCMS showed 80% desired product. The reaction was then diluted by the addition of EA (150 mL). The resulting solution was washed with brine (3×70 mL) and the organic layers were combined, dried over anhydrous sodium sulfate, filtrated and the filtrate was concentrated in vacuo to dryness. The residue was applied onto a silica gel column chromatography with dichloromethane/methanol (10/1). This resulted in 7-(2-fluoroethyl)-3-iodo-2-(6-methoxy-1,5-naphthyridin-4-yl)-1H,5H,6H,7H-pyrrolo[3,2-c]pyridin-4-one (254 mg, 50.1%) as a yellow solid.

LC-MS: (M+H)⁺ found: 467.0.

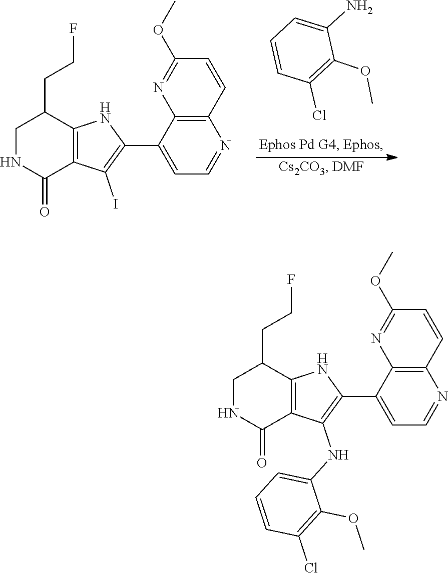

Into a 8-mL vial purged and maintained with an inert atmosphere of nitrogen, were placed 7-(2-fluoroethyl)-3-iodo-2-(6-methoxy-1,5-naphthyridin-4-yl)-1H,5H,6H,7H-pyrrolo[3,2-c]pyridin-4-one (239 mg, 0.513 mmol, 1.0 equiv), DMF (10 mL), 3-chloro-2-methoxyaniline (88.9 mg, 0.564 mmol, 1.1 equiv), Ephos Pd G4 (188.3 mg, 0.205 mmol, 0.4 equiv), Ephos (109.7 mg, 0.205 mmol, 0.4 equiv) and Cs₂CO₃ (501 mg, 1.54 mmol, 3.0 equiv) at ambient temperature. The resulting mixture was stirred for 3 h at 50° C. The reaction progress was monitored by LCMS. The reaction was then diluted by the addition of EA (150 mL). The resulting solution was washed with brine (3×80 mL) and the organic layers were combined, dried over anhydrous sodium sulfate, filtrated and the filtrate was concentrated in vacuo to dryness. The residue was applied onto a silica gel column chromatography with dichloromethane/methanol (10/1). This resulted in 3-[(3-chloro-2-methoxyphenyl)amino]-7-(2-fluoroethyl)-2-(6-methoxy-1,5-naphthyridin-4-yl)-1H,5H,6H,7H-pyrrolo[3,2-c]pyridin-4-one (240 mg, 94.4%) as a yellow solid.

LC-MS: (M+H)⁺ found: 496.1.

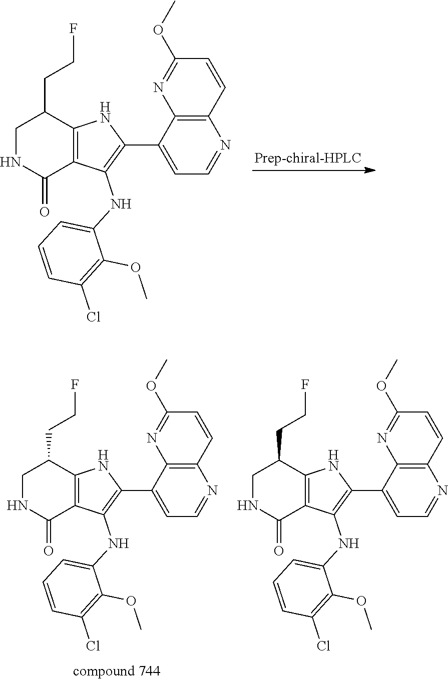 [SMILES: COc1ccc2nccc(-c3[nH]c4c(c3Nc3cccc(Cl)c3OC)C(=O)NCC4CCF)c2n1 ; COc1ccc2nccc(-c3[nH]c4c(c3Nc3cccc(Cl)c3OC)C(=O)NC[C@@H]4CCF)c2n1 ; COc1ccc2nccc(-c3[nH]c4c(c3Nc3cccc(Cl)c3OC)C(=O)NC[C@H]4CCF)c2n1]

240 mg of 3-[(3-chloro-2-methoxyphenyl)amino]-7-(2-fluoroethyl)-2-(6-methoxy-1,5-naphthyridin-4-yl)-1H,5H,6H,7H-pyrrolo[3,2-c]pyridin-4-one was purified by Chiral-Prep-HPLC. The collected fractions were combined and concentrated under vacuum. The residue was lyophilized to give (S)-3-((3-chloro-2-methoxyphenyl)amino)-7-(2-fluoroethyl)-2-(6-methoxy-1,5-naphthyridin-4-yl)-1,5,6,7-tetrahydro-4H-pyrrolo[3,2-c]pyridin-4-one (Assumed) (57.9 mg, 0.038 mmol, 48.3% yield) as a yellow solid.

LC-MS: (M+H)⁺ found: 496.1.

¹H NMR (300 MHz, DMSO-d₆) 12.17 (s, 1H), 8.59 (d, J=4.8 Hz, 1H), 8.31 (d, J=9.1 Hz, 1H), 7.82 (s, 1H), 7.53 (d, J=4.9 Hz, 1H), 7.37 (d, J=9.1 Hz, 1H), 7.29 (s, 1H), 6.76-6.61 (m, 2H), 6.17 (dd, J=7.5, 2.2 Hz, 1H), 4.79-4.43 (m, 2H), 4.18 (s, 3H), 3.89 (s, 3H), 3.69-3.51 (m, 1H), 3.29-3.18 (m, 2H), 2.24-1.96 (m, 2H).

### Example 17. (R)-3-((3-chloro-2-methoxyphenyl)amino)-7-(2-fluoroethyl)-2-(6-methoxy-1,5-naphthyridin-4-yl)-1,5,6,7-tetrahydro-4H-pyrrolo[3,2-c]pyridin-4-one (compound 745)

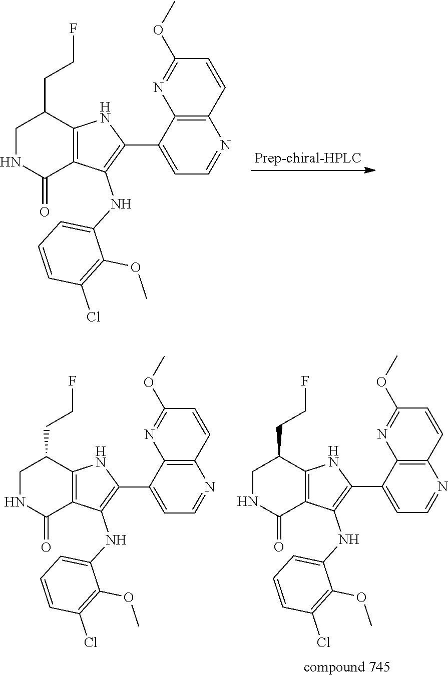 [SMILES: COc1ccc2nccc(-c3[nH]c4c(c3Nc3cccc(Cl)c3OC)C(=O)NCC4CCF)c2n1 ; COc1ccc2nccc(-c3[nH]c4c(c3Nc3cccc(Cl)c3OC)C(=O)NC[C@@H]4CCF)c2n1 ; COc1ccc2nccc(-c3[nH]c4c(c3Nc3cccc(Cl)c3OC)C(=O)NC[C@H]4CCF)c2n1]

240 mg of 3-[(3-chloro-2-methoxyphenyl)amino]-7-(2-fluoroethyl)-2-(6-methoxy-1,5-naphthyridin-4-yl)-1H,5H,6H,7H-pyrrolo[3,2-c]pyridin-4-one was purified by Chiral-Prep-HPLC. The collected fractions were combined and concentrated under vacuum. The residue was lyophilized to give (R)-3-((3-chloro-2-methoxyphenyl)amino)-7-(2-fluoroethyl)-2-(6-methoxy-1,5-naphthyridin-4-yl)-1,5,6,7-tetrahydro-4H-pyrrolo[3,2-c]pyridin-4-one (Assumed) (65.0 mg, 54.2% yield) as a yellow solid.

LC-MS: (M+H)⁺ found: 496.1.

¹H NMR (300 MHz, DMSO-d₆) δ 12.17 (s, 1H), 8.59 (d, J=4.8 Hz, 1H), 8.31 (d, J=9.1 Hz, 1H), 7.82 (s, 1H), 7.53 (d, J=4.9 Hz, 1H), 7.37 (d, J=9.1 Hz, 1H), 7.29 (s, 1H), 6.76-6.61 (m, 2H), 6.17 (dd, J=7.5, 2.2 Hz, 1H), 4.79-4.62 (m, 1H), 4.62-4.43 (m, 1H), 4.18 (s, 3H), 3.88 (s, 3H), 3.61 (d, J=10.8 Hz, 1H), 3.33-3.14 (m, 2H), 2.24-1.96 (m, 2H).

### Example 18. 3′-[(3-chloro-2-methoxyphenyl)amino]-2′-[6-(oxetan-3-yloxy)-1,5-naphthyridin-4-yl]-5′,6′-dihydro-1′H-spiro[cyclopropane-1,7′-pyrrolo[3,2-c]pyridin]-4′-one (compound 777)

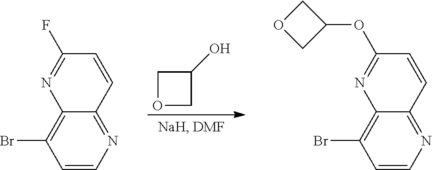

To a solution of cyclopropanol (767.46 mg, 13.213 mmol, 1.5 equiv) in THF (10 mL) was added NaH (704.67 mg, 17.618 mmol, 2 equiv, 60%) at 0 degrees C. The mixture was stirred for 15 min. 8-bromo-2-fluoro-1,5-naphthyridine (2 g, 8.809 mmol, 1.00 equiv) was added and the mixture was allowed to warm to RT and stirred for 2 h. The reaction mixture was quenched by water and extracted with DCM (3*25 mL). The aqueous layer was extracted with EtOAc (3×10 mL). The residue was purified by silica gel column chromatography, eluted with PE/EA (10:1) to afford 8-bromo-2-cyclopropoxy-1,5-naphthyridine (1.7 g, 72.79%) as a yellow oil.

LC-MS: M+H found: 282.

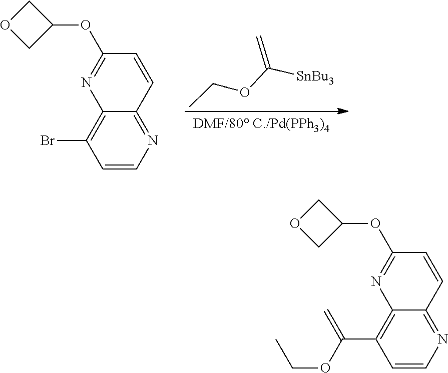

To a stirred solution of 8-bromo-2-(oxetan-3-yloxy)-1,5-naphthyridine (1 g, 3.557 mmol, 1.00 equiv) and tributyl(1-ethoxyethenyl)stannane (1.54 g, 4.268 mmol, 1.2 equiv) in 1,4-dioxane (20 mL) was added Pd(PPh₃)₄ (0.62 g, 0.534 mmol, 0.15 equiv) in portions at room temperature under nitrogen atmosphere. The resulting mixture was stirred for overnight at 90° C. under nitrogen atmosphere. Desired product could be detected by LCMS. The aqueous layer was extracted with EtOAc (3×10 mL). The residue was purified by silica gel column chromatography, eluted with PE/EA (1:1) to afford 8-(1-ethoxyethenyl)-2-(oxetan-3-yloxy)-1,5-naphthyridine (780 mg, 80.52%) as a yellow solid.

LC-MS: (M+H)⁺ found: 273.

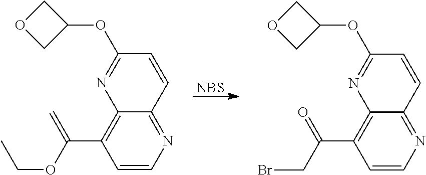

To a stirred solution of 8-(1-ethoxyethenyl)-2-(oxetan-3-yloxy)-1,5-naphthyridine (765 mg, 2.809 mmol, 1.00 equiv) in DMF (10 mL) was added NBS (500.02 mg, 2.809 mmol, 1 equiv) in portions at room temperature under nitrogen atmosphere. The resulting mixture was stirred for 1 h at room temperature under nitrogen atmosphere. Desired product could be detected by LCMS. The aqueous layer was extracted with EtOAc (3×10 mL). The resulting mixture was concentrated under reduced pressure to afford 2-bromo-1-[6-(oxetan-3-yloxy)-1,5-naphthyridin-4-yl]ethanone (720 mg, 79.31%) as a yellow solid

LC-MS: (M+H)⁺ found: 324.

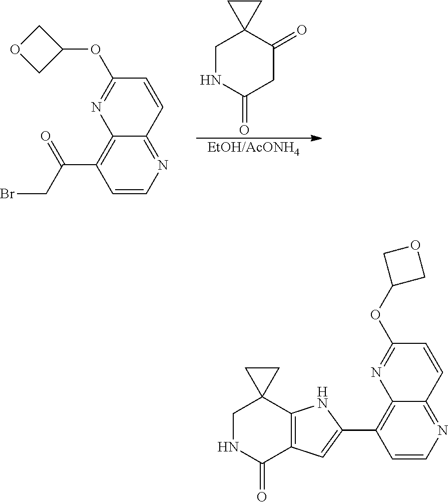

To a stirred solution of 5-azaspiro[2.5]octane-6,8-dione (10 mg, 0.072 mmol, 1.00 equiv) and AcONH₄ (33.24 mg, 0.432 mmol, 6 equiv) in EtOH (1 mL) was added 2-bromo-1-[6-(oxetan-3-yloxy)-1,5-naphthyridin-4-yl]ethanone (34.83 mg, 0.108 mmol, 1.5 equiv) dropwise at room temperature under nitrogen atmosphere. The resulting mixture was stirred for 2 h at 50° C. under nitrogen atmosphere. Desired product could be detected by LCMS. The aqueous layer was extracted with EtOAc (3×10 mL). The residue was purified by silica gel column chromatography, eluted with PE/EA (1:1) to afford 2′-[6-(oxetan-3-yloxy)-1,5-naphthyridin-4-yl]-5′,6′-dihydro-1′H-spiro[cyclopropane-1,7′-pyrrolo[3,2-c]pyridin]-4′-one (1.2 g, 42.67%) as a yellow solid.

LC-MS: (M+H)⁺ found: 363.

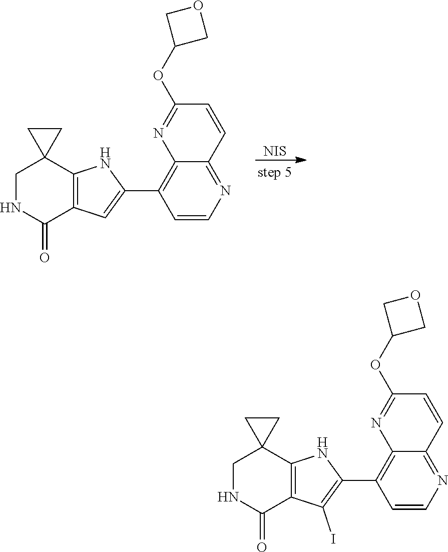

To a stirred solution of 2′-[6-(oxetan-3-yloxy)-1,5-naphthyridin-4-yl]-5′,6′-dihydro-1′H-spiro[cyclopropane-1,7′-pyrrolo[3,2-c]pyridin]-4′-one (900 mg, 2.484 mmol, 1.00 equiv) in DMF (15 mL) was added NIS (558.75 mg, 2.484 mmol, 1 equiv) dropwise at room temperature under nitrogen atmosphere. The resulting mixture was stirred for 1 h at room temperature under nitrogen atmosphere Desired product could be detected by LCMS. The aqueous layer was extracted with EtOAc (3×10 mL). The resulting mixture was concentrated under reduced pressure to afford 3′-iodo-2′-[6-(oxetan-3-yloxy)-1,5-naphthyridin-4-yl]-5′,6′-dihydro-1′H-spiro[cyclopropane-1,7′-pyrrolo[3,2-c]pyridin]-4′-one (870 mg, 71.74%) as a brown solid

LC-MS: (M+H)⁺ found: 489.

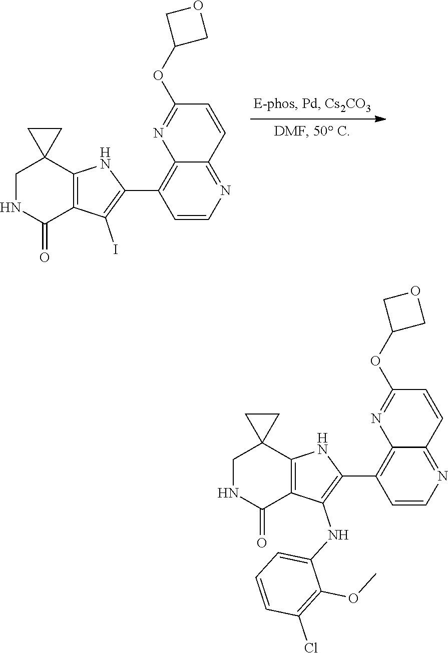 [SMILES: O=C1NCC2(CC2)c2[nH]c(-c3ccnc4ccc(OC5COC5)nc34)c(I)c21 ; COc1c(Cl)cccc1Nc1c(-c2ccnc3ccc(OC4COC4)nc23)[nH]c2c1C(=O)NCC21CC1]

To a stirred mixture of 3′-iodo-2′-[6-(oxetan-3-yloxy)-1,5-naphthyridin-4-yl]-5′,6′-dihydro-1′H-spiro[cyclopropane-1,7′-pyrrolo[3,2-c]pyridin]-4′-one (50 mg, 0.102 mmol, 1 equiv) and Cs₂CO₃ (66.73 mg, 0.204 mmol, 2 equiv) in DMF (10 mL) were added 3-chloro-2-methoxyaniline (19.37 mg, 0.122 mmol, 1.2 equiv) and EPhos (10.95 mg, 0.020 mmol, 0.2 equiv) and EPhos Pd G4 (9.41 mg, 0.010 mmol, 0.1 equiv) in portions at room temperature under nitrogen atmosphere. The resulting mixture was stirred for 3 h at 50 degrees C. under nitrogen atmosphere. The aqueous layer was extracted with EtOAc (3×10 mL). The resulting mixture was concentrated under reduced pressure. The residue was purified by reverse flash chromatography to afford 3′-[(3-chloro-2-methoxyphenyl)amino]-2′-[6-(oxetan-3-yloxy)-1,5-naphthyridin-4-yl]-5′,6′-dihydro-1′H-spiro[cyclopropane-1,7′-pyrrolo[3,2-c]pyridin]-4′-one (6.1 mg, 11.50%) as a yellow solid with the following conditions: column, C18 silica gel; mobile phase, MeOH in water, 10% to 50% gradient in 10 min; detector, UV 254 nm.

¹H NMR (300 MHz, Chloroform-d) δ 11.57 (s, 1H), 8.84 (s, 1H), 8.49 (s, 1H), 8.35 (s, 1H), 7.37 (d, J=7.9 Hz, 1H), 6.88 (d, J=7.6 Hz, 1H), 6.67 (t, J=7.9 Hz, 1H), 6.12 (d, J=7.5 Hz, 1H), 5.88 (s, 1H), 5.67 (s, 1H), 5.11 (s, 2H), 4.94 (s, 2H), 4.14 (s, 3H), 3.55 (s, 2H), 1.38 (s, 4H), 1.28 (s, 1H).

### Example 19. 3′-[(3-chloro-2-methoxyphenyl)amino]-2′-(3-fluoropyridin-4-yl)-5′,6′-dihydro-1′H-spiro[oxane-4,7′-pyrrolo[3,2-c]pyridin]-4′-one (compound 778)

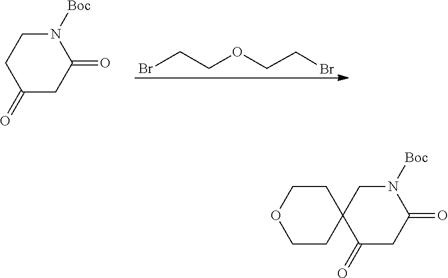

To a solution of tert-butyl 2,4-dioxopiperidine-1-carboxylate (40 g, 187.588 mmol, 1.00 equiv), in dry THF (600 mL) and cooled to −20 degrees C. under nitrogen, LiHMDS (188.33 g, 1125.528 mmol, 6 equiv) (0.47 mL of 1 M solution in THF) was added dropwise. After 20 min stirring, 1-bromo-2-(2-bromoethoxy)ethane (174.02 g, 750.352 mmol, 4 equiv) were added and the solution was stirred at −20° C. for 5 hours. The mixture was neutralized to pH 7 with 5% aq KHSO₄. The aqueous layer was extracted with DCM (5×1000 mL). The collected organic layers were washed with water, dried over Na2SO4 and evaporated to dryness. The residue was purified by reverse flash chromatography with the following conditions: column, C18 silica gel; mobile phase, MeOH in water, 10% to 50%16 gradient in 10 min; detector, UV 254 nm to afford tert-butyl 3,5-dioxo-9-oxa-2-azaspiro[5.5]undecane-2-carboxylate (5 g, 9.41%) as a light yellow solid.

LC-MS: M+H found: 284.

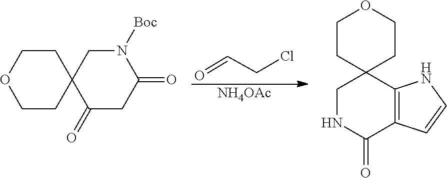

A solution of tert-butyl 3,5-dioxo-9-oxa-2-azaspiro[5.5]undecane-2-carboxylate (1.2 g, 4.235 mmol, 1.00 equiv) and chloroacetaldehyde (0.83 g, 4.235 mmol, 1 equiv), NH₄OAc (8.16 g, 105.875 mmol, 25 equiv) in EtOH (12 mL, 206.562 mmol, 48.77 equiv) was stirred for 24 h at 80 degrees C. The aqueous layer was extracted with EA (3×200 mL). The residue was purified by reverse flash chromatography with the following conditions: column, silica gel; mobile phase, MeCN in water; detector, UV 254 nm to afford 5′,6′-dihydro-1′H-spiro[oxane-4,7′-pyrrolo[3,2-c]pyridin]-4′-one (600 mg, 68.69%) as a yellow solid.

LC-MS: (M+H)⁺ found: 207.

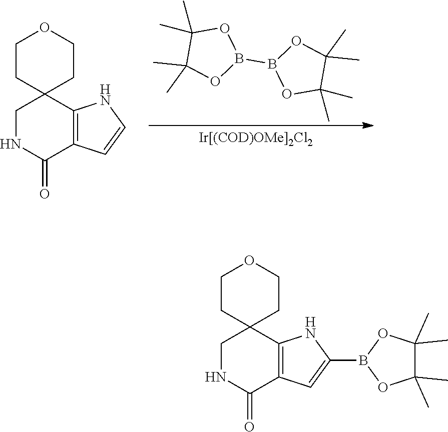

To a solution of 5′,6′-dihydro-1′H-spiro[oxane-4,7′-pyrrolo[3,2-c]pyridin]-4′-one (285 mg, 1.382 mmol, 1.00 equiv) and bis(pinacolato)diboron (701.81 mg, 2.764 mmol, 2 equiv) in dioxane (5 mL, 59.020 mmol, 42.71 equiv) were added 4,4′-di-tert-butyl-2,2′-dipyridyl (22.25 mg, 0.083 mmol, 0.06 equiv) and (1,5-cyclooctadiene) (methoxy) iridium (I) dimer (27.48 mg, 0.041 mmol, 0.03 equiv). After stirring for overnight at 50 degrees C. under a nitrogen atmosphere, the resulting mixture was concentrated under reduced pressure. The crude product was used in the next step directly without further purification.

LC-MS: (M+H)⁺ found: 333.

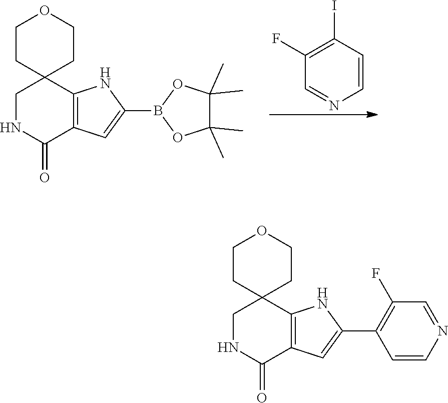 [SMILES: CC1(C)OB(c2cc3c([nH]2)C2(CCOCC2)CNC3=O)OC1(C)C ; Fc1cnccc1I ; O=C1NCC2(CCOCC2)c2[nH]c(-c3ccncc3F)cc21]

To a solution of 2′-(4,4,5,5-tetramethyl-1,3,2-dioxaborolan-2-yl)-5′,6′-dihydro-1′H-spiro[oxane-4,7′-pyrrolo[3,2-c]pyridin]-4′-one (1.1 g, 3.311 mmol, 1.00 equiv) and 3-fluoro-4-iodopyridine (0.81 g, 3.642 mmol, 1.1 equiv) in dioxane (15 mL, 177.061 mmol, 53.47 equiv) were added K₂CO₃ (1.37 g, 9.933 mmol, 3 equiv) and Pd(dppf)Cl₂ (0.24 g, 0.331 mmol, 0.1 equiv). After stirring for 3 h at 80 degrees C. under a nitrogen atmosphere, the resulting mixture was concentrated under reduced pressure. The aqueous layer was extracted with EA (8×100 mL). The residue was purified by reverse flash chromatography with the following conditions; column, silica gel; mobile phase, MeCN in water; detector, UV 254 nm to afford 2′-(3-fluoropyridin-4-yl)-5′,6′-dihydro-1′H-spiro[oxane-4,7′-pyrrolo[3,2-c]pyridin]-4′-one (280 mg, 28.06%) as a brown solid.

LC-MS: (M+H)⁺ found: 302.

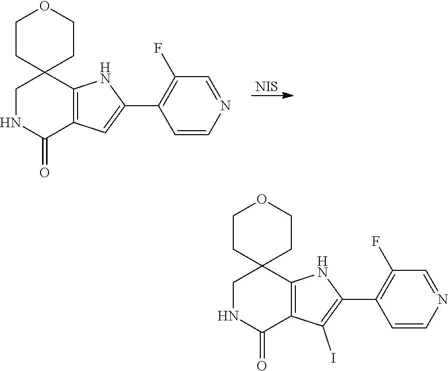

A solution of 2′-(3-hydroxypyridin-4-yl)-5′,6′-dihydro-1′H-spiro[oxane-4,7′-pyrrolo[3,2-c]pyridin]-4′-one (220 mg, 0.735 mmol, 1.00 equiv) and (iodoamino)sulfanyl (127.13 mg, 0.735 mmol, 1 equiv) in DMF (5 mL, 64.609 mmol, 87.91 equiv) was stirred for 1 h at rt. The aqueous layer was extracted with EA (8×100 mL). This resulted in 2′-(3-fluoropyridin-4-yl)-3′-iodo-5′,6′-dihydro-1′H-spiro[oxane-4,7′-pyrrolo[3,2-c]pyridin]-4′-one (310 mg, 98.73%) as a brown oil.

LC-MS: (M+H)⁺ found: 428.

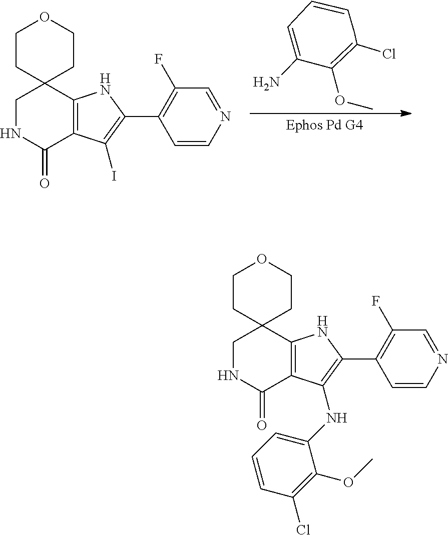 [SMILES: O=C1NCC2(CCOCC2)c2[nH]c(-c3ccncc3F)c(I)c21 ; COC1=C(Cl)CC=CC=C1N ; COc1c(Cl)cccc1Nc1c(-c2ccncc2F)[nH]c2c1C(=O)NCC21CCOCC1]

To a solution of 2′-(3-fluoropyridin-4-yl)-3′-iodo-5′,6′-dihydro-1′H-spiro[oxane-4,7′-pyrrolo[3,2-c]pyridin]-4′-one (200 mg, 0.468 mmol, 1.00 equiv) and 3-chloro-2-methoxyaniline (88.54 mg, 0.562 mmol, 1.2 equiv) in DMF (8 mL, 103.374 mmol, 220.82 equiv) were added Cs₂CO₃ (305.06 mg, 0.936 mmol, 2 equiv), EPhos (50.07 mg, 0.094 mmol, 0.2 equiv) and EPhos Pd G4 (43.00 mg, 0.047 mmol, 0.1 equiv). After stirring for 2 h at 50 degrees C. under a nitrogen atmosphere, the resulting mixture was concentrated under reduced pressure. The aqueous layer was extracted with EA (5×50 mL). The residue was purified by reverse flash chromatography with the following conditions: column, silica gel; mobile phase, MeCN in water; detector, UV 254 nm to afford 3′-[(3-chloro-2-methoxyphenyl)amino]-2′-(3-fluoropyridin-4-yl)-5′,6′-dihydro-1′H-spiro[oxane-4,7′-pyrrolo[3,2-c]pyridin]-4′-one (24.4 mg, 11.32%) as a light yellow solid.

LC-MS: (M+H)⁺ found: 457.

¹H NMR (300 MHz, DMSO-d6) δ 11.46 (s, 1H), 8.49 (d, J=2.6 Hz, 1H), 8.34 (dd, J=5.1, 1.2 Hz, 1H), 7.66 (s, 1H), 7.48 (dd, J=6.7, 5.0 Hz, 1H), 7.30 (s, 1H), 6.68-6.57 (m, 2H), 6.09 (dd, J=7.6, 2.1 Hz, 1H), 3.84 (s, 5H), 3.61-3.47 (m, 4H), 2.13 (td, J=13.0, 4.8 Hz, 2H), 1.63 (d, J=13.3 Hz, 2H).

### Example 20. 3′-[(3-chloro-2-methoxyphenyl)amino]-2′-(3-fluoropyridin-4-yl)-5′,6′-dihydro-1′H-spiro[oxetane-3,7′-pyrrolo[3,2-c]pyridin]-4′-one (compound 748)

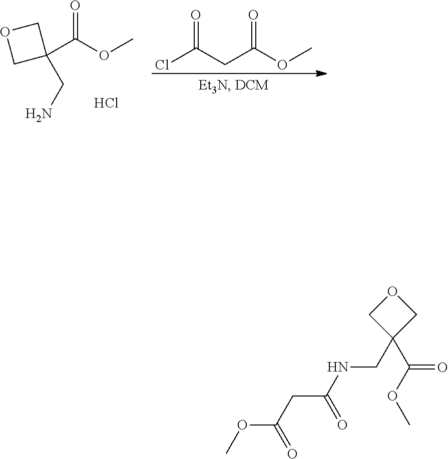

To a stirred mixture of methyl 3-(aminomethyl)oxetane-3-carboxylate hydrochloride (4 g, 22.024 mmol, 1 equiv) and Et3N (6.68 g, 66.072 mmol, 3 equiv) in DCM (50 mL) was added methyl 3-chloro-3-oxopropanoate (3 g, 22.024 mmol, 1 equiv) dropwise at 0° C. under nitrogen atmosphere. The resulting mixture was stirred for 1 h at room temperature under nitrogen atmosphere. The resulting mixture was extracted with CH₂Cl₂ (2×250 mL). The combined organic layers were dried over anhydrous Na₂SO₄. After filtration, the filtrate was concentrated under reduced pressure. to afford methyl 3-[(3-methoxy-3-oxopropanamido)methyl]oxetane-3-carboxylate (5 g, 92.58%) as a light yellow oil.

LC-MS: M+H found: 246.

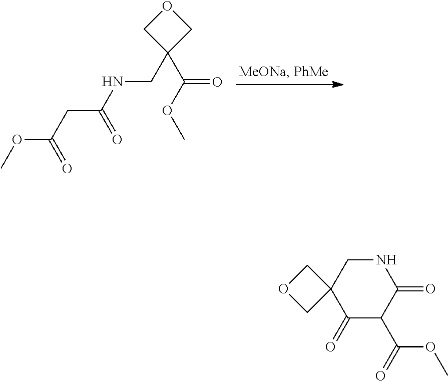 [SMILES: COC(=O)CC(=O)NCC1(C(=O)OC)COC1 ; COC(=O)C1C(=O)NCC2(COC2)C1=O]

To a stirred mixture of methyl 3-[(3-methoxy-3-oxopropanamido)methyl]oxetane-3-carboxylate (5 g, 20.389 mmol, 1 equiv) in MeOH (20 mL) in Toluene (30 mL) was added MeONa (2.20 g, 40.778 mmol, 2 equiv) portion-wise at room temperature under nitrogen atmosphere. The resulting mixture was stirred for overnight at 80° C. under nitrogen atmosphere. The mixture was acidified to pH 7. The resulting mixture was concentrated under vacuum. The residue was purified by silica gel column chromatography, eluted with CH₂Cl₂/MeOH (10:1) to afford methyl 7,9-dioxo-2-oxa-6-azaspiro[3.5]nonane-8-carboxylate (2 g, 46.01%) as an off-white solid.

LC-MS: (M+H)⁺ found: 214.

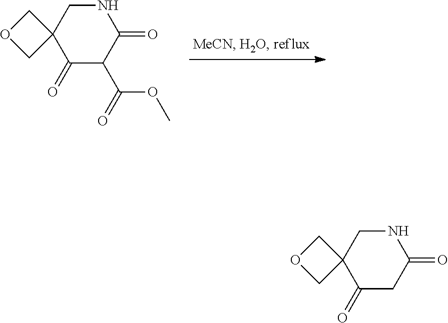

A mixture of methyl 7,9-dioxo-2-oxa-6-azaspiro[3.5]nonane-8-carboxylate (2 g, 9.381 mmol, 1 equiv) and H₂O (2 mL) in MeCN (20 mL) was stirred for 5 h at 90° C. under nitrogen atmosphere. Desired product could be detected by LCMS. The resulting mixture was concentrated under reduced pressure to afford 2-oxa-6-azaspiro[3.5]nonane-7,9-dione (1.4 g, 96.18%) as a yellow solid. The crude product was used in the next step directly without further purification.

LC-MS: (M+H)⁺ found 156.

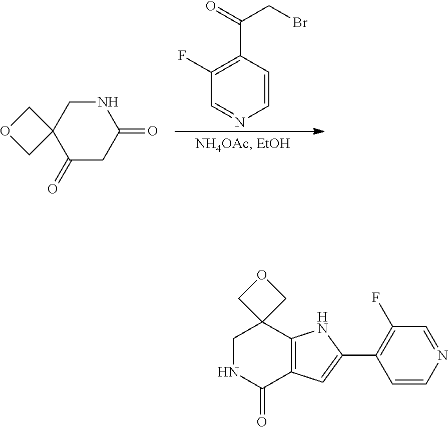 [SMILES: O=C1CC(=O)C2(CN1)COC2 ; O=C(CBr)c1ccncc1F ; O=C1NCC2(COC2)c2[nH]c(-c3ccncc3F)cc21]

To a stirred mixture of 2-oxa-6-azaspiro[3.5]nonane-7,9-dione (1.4 g, 9.023 mmol, 1 equiv) and 2-bromo-1-(3-fluoropyridin-4-yl)ethanone (1.97 g, 9.023 mmol, 1 equiv) in EtOH (15 mL) was added EtOAc (3.18 g, 36.092 mmol, 4 equiv) in portions at room temperature under nitrogen atmosphere. The resulting mixture was stirred for overnight at 50° C. under nitrogen atmosphere. The resulting mixture was extracted with EtOAc (2×150 mL). The combined organic layers were dried over anhydrous Na₂SO₄. After filtration, the filtrate was concentrated under reduced pressure. The residue was purified by reverse flash chromatography with the following conditions: column, silica gel; mobile phase, MeCN in water, 10% to 50% gradient in 10 min; detector, UV 254 nm. to afford 2′-(3-fluoropyridin-4-yl)-5′,6′-dihydro-1′H-spiro[oxetane-3,7′-pyrrolo[3,2-c]pyridin]-4′-one (150 mg, 6.08%) as a yellow solid.

LC-MS: (M+H)⁺ found: 274.

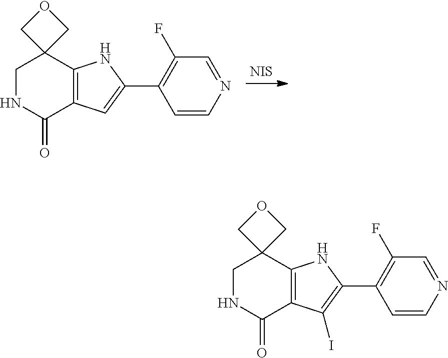

To a stirred mixture of 2′-(3-fluoropyridin-4-yl)-5′,6′-dihydro-1′H-spiro[oxetane-3,7′-pyrrolo[3,2-c]pyridin]-4′-one (150 mg, 0.549 mmol, 1.00 equiv) in DMF (3 mL) was added NIS (123.50 mg, 0.549 mmol, 1 equiv) slowly at 0° C. under nitrogen atmosphere. The resulting mixture was stirred for 1 h at room temperature under nitrogen atmosphere. The residue was purified by reverse flash chromatography with the following conditions: column, silica gel; mobile phase, MeCN in water, 10/to 50% gradient in 10 min; detector, UV 254 nm. to afford 2′-(3-fluoropyridin-4-yl)-3′-iodo-5′,6′-dihydro-1′H-spiro[oxetane-3,7′-pyrrolo[3,2-c]pyridin]-4′-one (65 mg, 29.67%) as a yellow solid.

LC-MS: (M+H)⁺ found: 400.

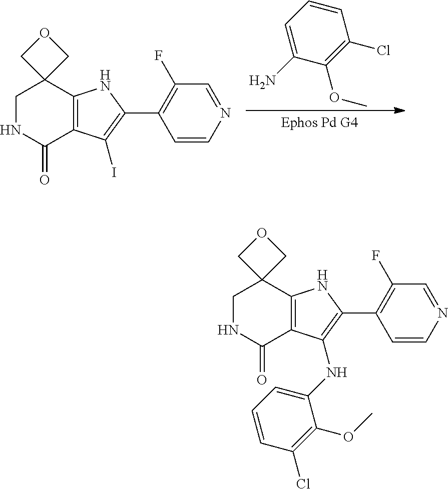

Into a 2 mL sealed tube were added 2′-(3-fluoropyridin-4-yl)-3′-iodo-5′,6′-dihydro-1′H-spiro[oxetane-3,7′-pyrrolo[3,2-c]pyridin]-4′-one (60 mg, 0.150 mmol, 1.00 equiv), 3-chloro-2-methoxyaniline (28.43 mg, 0.180 mmol, 1.2 equiv), Ephos Pd G4 (20.71 mg, 0.022 mmol, 0.15 equiv), EPhos (24.12 mg, 0.045 mmol, 0.3 equiv), Cs₂CO₃ (97.95 mg, 0.300 mmol, 2 equiv), and DMF (2 mL) at room temperature. The resulting mixture was stirred for 2 h at 50 degrees C. under nitrogen atmosphere. Desired product could be detected by LCMS. The residue was purified by reverse flash chromatography with the following conditions: column, C18 silica gel; mobile phase, MeOH in water, 10% to 60% gradient in 15 to afford 3′-[(3-chloro-2-methoxyphenyl)amino]-2′-(3-fluoropyridin-4-yl)-5′,6′-dihydro-1′H-spiro[oxetane-3,7′-pyrrolo[3,2-c]pyridin]-4′-one (11.1 mg, 17.03%) as a white solid.

¹H NMR (300 MHz, DMSO-d6) δ 3.75 (d, 2H), 3.83 (s, 3H), 4.51 (d, 2H), 4.96 (d, 2H), 6.11 (dd, 1H), 6.55-6.70 (m, 2H), 7.38 (s, 1H), 7.49-7.59 (m, 2H), 8.38 (dd, 1H), 8.51 (d, 1H), 12.12 (s, 1H).

### Example 21. methyl 3′-[(3-chloro-2-methoxyphenyl)amino]-2′-(3-fluoropyridin-4-yl)-4′-oxo-5′,6′-dihydro-1′H-spiro[azetidine-3,7′-pyrrolo[3,2-c]pyridine]-1-carboxylate (compound 490)

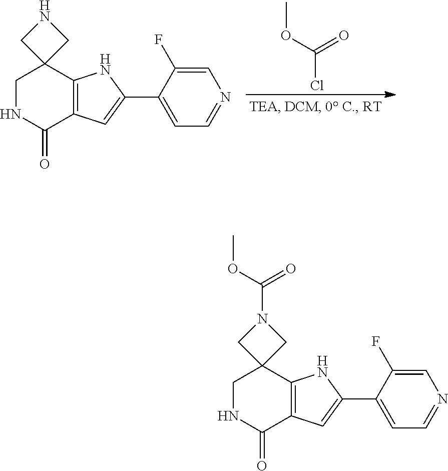

To a stirred mixture of 2′-(3-fluoropyridin-4-yl)-5′,6′-dihydro-1′H-spiro[azetidine-3,7′-pyrrolo[3,2-c]pyridin]-4′-one (200 mg, 0.735 mmol, 1.00 equiv) and TEA (222.98 mg, 2.205 mmol, 3 equiv) in DCM (5 mL) was added methyl chloroformate (83.29 mg, 0.882 mmol, 1.2 equiv) dropwise at 0 degrees C. under nitrogen atmosphere. The resulting mixture was stirred for 2 h at room temperature under nitrogen atmosphere. The reaction was quenched by the addition of Water at 0 degrees C. The resulting mixture was concentrated under reduced pressure. The residue was purified by reverse flash chromatography with the following conditions: column, C18 silica gel; mobile phase, MeCN in water, 10% to 30% gradient in 30 min; detector, UV 220 nm. This resulted in methyl 2′-(3-fluoropyridin-4-yl)-4′-oxo-5′,6′-dihydro-1′H-spiro[azetidine-3,7′-pyrrolo[3,2-c]pyridine]-1-carboxylate (120 mg, 49.46%) as a white solid.

LC-MS: M+H found: 331.

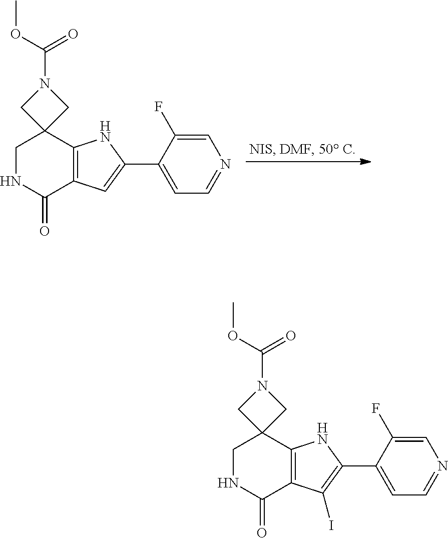 [SMILES: COC(=O)N1CC2(CNC(=O)c3cc(-c4ccncc4F)[nH]c32)C1 ; COC(=O)N1CC2(CNC(=O)c3c2[nH]c(-c2ccncc2F)c3I)C1]

A mixture of methyl 2′-(3-fluoropyridin-4-yl)-4′-oxo-5′,6′-dihydro-1′H-spiro[azetidine-3,7′-pyrrolo[3,2-c] pyridine]-1-carboxylate (140 mg, 0.424 mmol, 1.00 equiv) and NIS (104.89 mg, 0.466 mmol, 1.1 equiv) in DMF (3 mL) was stirred for overnight at room temperature under nitrogen atmosphere. The resulting mixture was washed with water. The resulting mixture was extracted with CH₂Cl₂. The combined organic layers were washed with brine, dried over anhydrous Na₂SO₄. After filtration, the filtrate was concentrated under reduced pressure. The residue was purified by Prep-TLC (CH₂Cl₂/MeOH 20:1) to afford methyl 2′-(3-fluoropyridin-4-yl)-3′-iodo-4′-oxo-5′,6′-dihydro-1′H-spiro[azetidine-3,7′-pyrrolo[3,2-c] pyridine]-1-carboxylate (66 mg, 34.13%) as a yellow solid.

LC-MS: (M+H)⁺ found: 457.

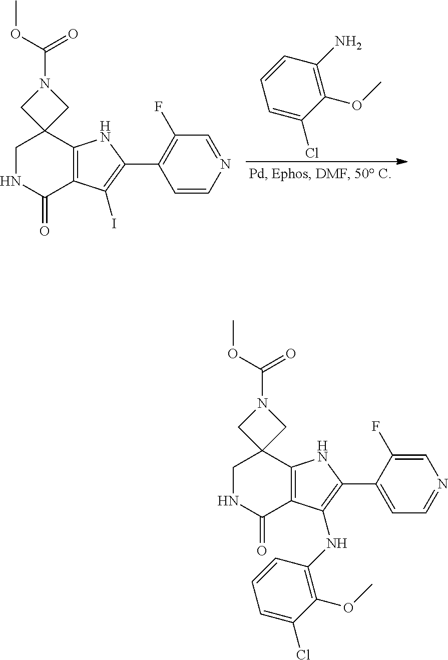

A mixture of methyl 2′-(3-fluoropyridin-4-yl)-3′-iodo-4′-oxo-5′,6′-dihydro-1′H-spiro[azetidine-3,7′-pyrrolo[3,2-c]pyridine]-1-carboxylate (60 mg, 0.132 mmol, 1.00 equiv), 3-chloro-2-methoxyaniline (31.09 mg, 0.198 mmol, 1.5 equiv), EPhos Pd G4 (12.08 mg, 0.013 mmol, 0.1 equiv), EPhos (14.07 mg, 0.026 mmol, 0.2 equiv), and Cs₂CO₃ (128.55 mg, 0.396 mmol, 3 equiv) in DMF (2 mL) was stirred for 1 h at 50 degrees C. under nitrogen atmosphere. The resulting mixture was diluted with water. The resulting mixture was extracted with CH₂Cl₂. The combined organic layers were dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 50% gradient in 30 min; detector, UV 254 nm. This resulted in methyl 3′-[(3-chloro-2-methoxyphenyl)amino]-2′-(3-fluoropyridin-4-yl)-4′-oxo-5′,6′-dihydro-1′H-spiro[azetidine-3,7′-pyrrolo[3,2-c]pyridine]-1-carboxylate (27 mg, 42.25%) as a light yellow solid.

LC-MS: (M+H)⁺ found: 486.

¹H NMR (300 MHz, Chloroform-d) δ 9.74 (d, J=6.9 Hz, 1H), 8.48 (d, J=3.6 Hz, 1H), 8.19 (d, J=5.2 Hz, 1H), 7.56 (s, 1H), 7.37 (dd, J=7.0, 5.2 Hz, 1H), 6.75 (dd, J=8.1, 1.5 Hz, 1H), 6.62 (t, J=8.1 Hz, 1H), 6.16 (dd, J=8.1, 1.5 Hz, 1H), 5.65 (s, 1H), 4.27 (d, J=8.9 Hz, 2H), 4.06 (d, J=19.8 Hz, 5H), 3.74 (d, J=16.8 Hz, 5H).

### Example 22. (3S)-3′-[(3-chloro-2-methoxyphenyl)amino]-2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (compound 500)

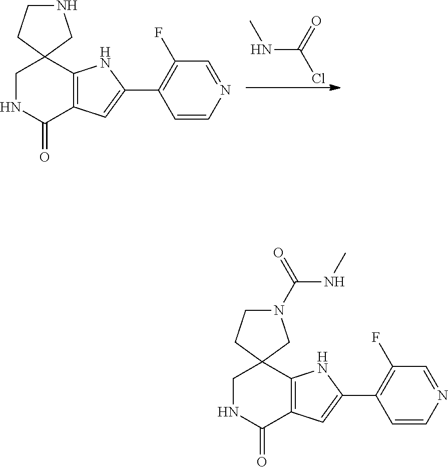

To a stirred mixture of 2′-(3-fluoropyridin-4-yl)-5′,6′-dihydro-1′H-spiro[pyrrolidine-3,7′-pyrrolo[3,2-c]pyridin]-4′-one (1 g, 3.493 mmol, 1 equiv) and N-methylcarbamoyl chloride (0.33 g, 3.493 mmol, 1 equiv) in DCM (20 mL) was added TEA (2.43 mL, 17.465 mmol, 5 equiv) in portions at room temperature. The resulting mixture was stirred for 1 h at room temperature. The resulting mixture was concentrated under reduced pressure. The residue was purified by reverse flash chromatography with the following conditions: column, C18 silica gel; mobile phase, MeCN in water, 10% to 100% gradient in 10 min; detector, UV 254 nm. This resulted in 2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (500 mg, 41.69%) as a yellow solid.

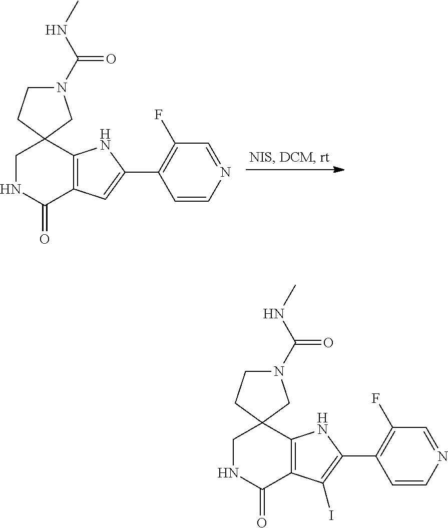 [SMILES: CNC(=O)N1CCC2(CNC(=O)c3cc(-c4ccncc4F)[nH]c32)C1 ; CNC(=O)N1CCC2(CNC(=O)c3c2[nH]c(-c2ccncc2F)c3I)C1]

To a stirred mixture of 2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (400 mg, 1.165 mmol, 1 equiv) in DCM (10 mL) was added NIS (262.10 mg, 1.165 mmol, 1 equiv) in portions at room temperature. The resulting mixture was stirred for 2 h at room temperature. The resulting mixture was concentrated under vacuum. The residue was purified by reverse flash chromatography with the following conditions: column, C18 silica gel; mobile phase, MeCN in water, 10% to 100% gradient in 10 min; detector, UV 254 nm. This resulted in 2′-(3-fluoropyridin-4-yl)-3′-iodo-N-methyl-4′-oxo-5′,6′-dihydro-1′H-spiro[pyrrolidine-3,7′-pyrrolo[3,2-c]pyridine]-1-carboxamide (260 mg, 47.56%) as a light yellow solid.

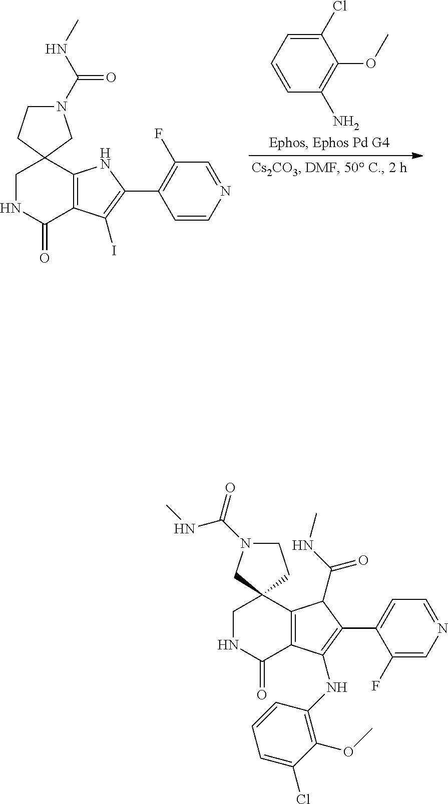

To a stirred mixture of 2′-(3-fluoropyridin-4-yl)-3′-iodo-N-methyl-4′-oxo-5′,6′-dihydro-1′H-spiro[pyrrolidine-3,7′-pyrrolo[3,2-c]pyridine]-1-carboxamide (100 mg, 0.213 mmol, 1 equiv) and 3-chloro-2-methoxyaniline (33.58 mg, 0.213 mmol, 1 equiv) in DMF (5 mL) were added Cs₂CO₃ (138.87 mg, 0.426 mmol, 2 equiv), EPhos (22.79 mg, 0.043 mmol, 0.2 equiv) and EPhos Pd G4 (78.30 mg, 0.085 mmol, 0.4 equiv) in portions at room temperature under nitrogen atmosphere. The resulting mixture was stirred for 2 h at 50° C. under nitrogen atmosphere. The resulting mixture was concentrated under vacuum. The residue was purified by reverse flash chromatography with the following conditions: column, C18 silica gel; mobile phase, MeCN in water, 0% to 100% gradient in 20 min; detector, UV 254 nm. The crude product (100 mg) was purified by Prep-HPLC with the following conditions (Column: CHIRALPAK IG, 2*25 cm, 5 μm; Mobile Phase A: Hex:DCM=3:1 (0.5% 2M NH₃-MeOH)-HPLC, Mobile Phase B: IPA-HPLC; Flow rate: 20 mL/min; Gradient: 30% B to 30% B in 17 min; Wave Length: 220/254 nm; RT1 (min): 7.27: RT2 (min): 13.92; Sample Solvent: ETOH:DCM=1:1; Injection Volume: 1 mL; Number Of Runs: 8) to afford (3S)-3′-[(3-chloro-2-methoxyphenyl)amino]-2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (39.1 mg, 33.35%) as a white solid.

LC-MS: M+H found: 499.

¹H NMR (400 MHz, Chloroform-d) δ 9.25 (d, J=8.6 Hz, 1H), 8.49-8.40 (m, 1H), 8.18 (dd, J=5.3, 1.3 Hz, 1H), 7.59 (s, 1H), 7.33 (dd, J=7.1, 5.3 Hz, 1H), 6.77 (dd, J=8.1, 1.5 Hz, 1H), 6.64 (t, J=8.1 Hz, 1H), 6.18 (dd, J=8.2, 1.5 Hz, 1H), 5.47 (s, 1H), 4.34 (d, J=4.9 Hz, 1H), 4.05 (s, 3H), 3.71 (d, J=10.6 Hz, 1H), 3.68-3.47 (m, 4H), 3.47-3.33 (m, 1H), 2.89-2.80 (m, 3H), 2.42-2.31 (m, 1H), 2.26 (ddd, J=12.9, 7.8, 5.0 Hz, 1H), 1.25 (s, 1H).

### Example 23. (3R)-3′-[(3-chloro-2-methoxyphenyl)amino]-2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (compound 499)

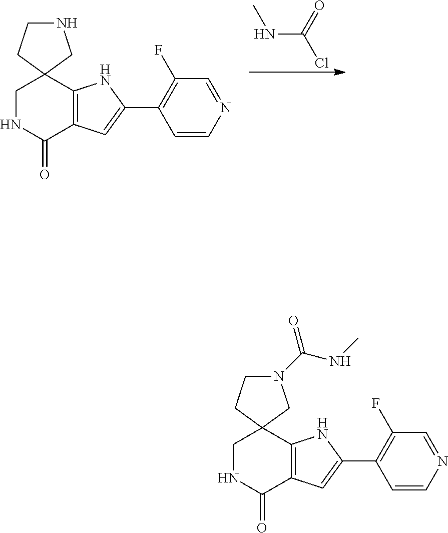

To a stirred mixture of 2′-(3-fluoropyridin-4-yl)-5′,6′-dihydro-1′H-spiro[pyrrolidine-3,7′-pyrrolo[3,2-c]pyridin]-4′-one (1 g, 3.493 mmol, 1 equiv) and N-methylcarbamoyl chloride (0.33 g, 3.493 mmol, 1 equiv) in DCM (20 mL) was added TEA (2.43 mL, 17.465 mmol, 5 equiv) in portions at room temperature. The resulting mixture was stirred for 1 h at room temperature. The resulting mixture was concentrated under reduced pressure. The residue was purified by reverse flash chromatography with the following conditions: column, C18 silica gel; mobile phase, MeCN in water, 10% to 100% gradient in 10 min; detector, UV 254 nm. This resulted in 2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (500 mg, 41.69%) as a yellow solid.

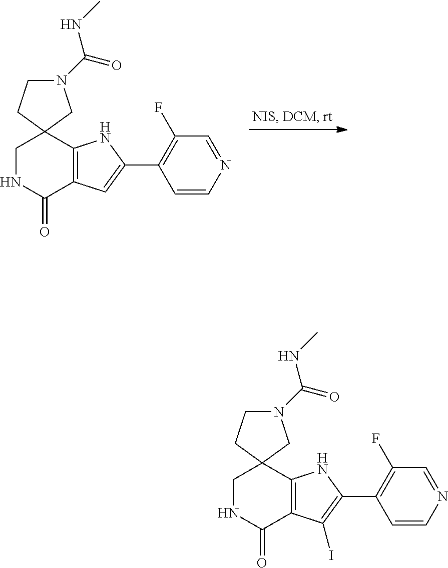 [SMILES: CNC(=O)N1CCC2(CNC(=O)c3cc(-c4ccncc4F)[nH]c32)C1 ; CNC(=O)N1CCC2(CNC(=O)c3c2[nH]c(-c2ccncc2F)c3I)C1]

To a stirred mixture of 2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (400 mg, 1.165 mmol, 1 equiv) in DCM (10 mL) was added NIS (262.10 mg, 1.165 mmol, 1 equiv) in portions at room temperature. The resulting mixture was stirred for 2 h at room temperature. The resulting mixture was concentrated under vacuum. The residue was purified by reverse flash chromatography with the following conditions: column, C18 silica gel, mobile phase, MeCN in water, 10% to 100% gradient in 10 min; detector, UV 254 nm. This resulted in 2′-(3-fluoropyridin-4-yl)-3′-iodo-N-methyl-4′-oxo-5′,6′-dihydro-1′H-spiro[pyrrolidine-3,7′-pyrrolo[3,2-c]pyridine]-1-carboxamide (260 mg, 47.56%) as a light yellow solid.

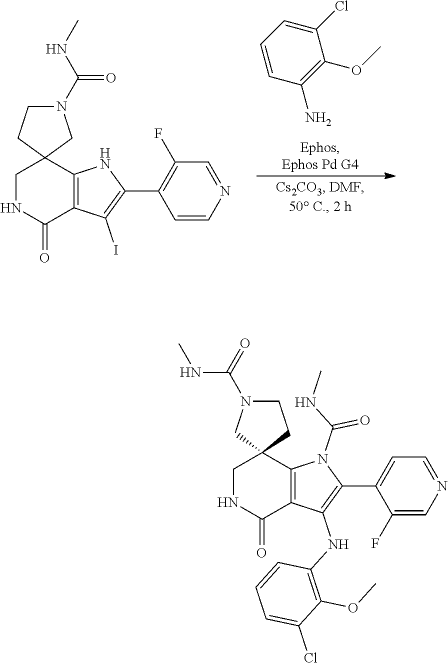

To a stirred mixture of 2′-(3-fluoropyridin-4-yl)-3′-iodo-N-methyl-4′-oxo-5′,6′-dihydro-1′H-spiro[pyrrolidine-3,7′-pyrrolo[3,2-c]pyridine]-1-carboxamide (100 mg, 0.213 mmol, 1 equiv) and 3-chloro-2-methoxyaniline (33.58 mg, 0.213 mmol, 1 equiv) in DMF (5 mL) were added Cs2CO3 (138.87 mg, 0.426 mmol, 2 equiv), EPhos (22.79 mg, 0.043 mmol, 0.2 equiv) and EPhos Pd G4 (78.30 mg, 0.085 mmol, 0.4 equiv) in portions at room temperature under nitrogen atmosphere. The resulting mixture was stirred for 2 h at 50° C. under nitrogen atmosphere. The resulting mixture was concentrated under vacuum. The residue was purified by reverse flash chromatography with the following conditions: column, C18 silica gel; mobile phase, MeCN in water, 0% to 100% gradient in 20 min; detector, UV 254 nm. The crude product (100 mg) was purified by Prep-HPLC with the following conditions (Column: CHIRALPAK IG, 2*25 cm, 5 μm; Mobile Phase A: Hex:DCM=3:1 (0.5% 2M NH3-MeOH)-HPLC, Mobile Phase B: IPA-HPLC; Flow rate: 20 mL/min; Gradient: 30% B to 30% B in 17 min; Wave Length: 220/254 nm; RT1 (min): 7.27; RT2 (min): 13.92; Sample Solvent: ETOH:DCM=1:1; Injection Volume: 1 mL; Number Of Runs: 8) to afford (3R)-3′-[(3-chloro-2-methoxyphenyl)amino]-2′-(3-fluoropyridin-4-yl)-N-methyl-4′-oxo-5′,6′-dihydro-1′H-spiro[pyrrolidine-3,7′-pyrrolo[3,2-c]pyridine]-1-carboxamide (24.8 mg, 23.11%) as a white solid.

LC-MS: M+H found: 499.

¹H NMR (400 MHz, Chloroform-d) δ 9.23 (d, J=8.6 Hz, 1H), 8.46 (d, J=3.9 Hz, 1H), 8.18 (dd, J=5.3, 1.3 Hz, 1H), 7.59 (s, 1H), 7.33 (dd, J=7.1, 5.2 Hz, 1H), 6.77 (dd, J=8.1, 1.5 Hz, 1H), 6.65 (t, J=8.1 Hz, 1H), 6.18 (dd, J=8.1, 1.5 Hz, 1H), 5.44 (s, 1H), 4.33 (d, J=4.9 Hz, 1H), 4.05 (s, 3H), 3.72 (d, J=10.6 Hz, 1H), 3.68-3.49 (m, 4H), 3.42 (dd, J=12.3, 3.1 Hz, 1H), 2.86 (d, J=4.6 Hz, 3H), 2.42-2.31 (m, 1H), 2.26 (ddd, J=13.0, 7.9, 4.9 Hz, 1H), 1.25 (s, 1H).

### Example 24. methyl (3R)-3′-[(3-chloro-2-methoxyphenyl)amino]-2′-(3-fluoropyridin-4-yl)-4′-oxo-5′,6′-dihydro-1′H-spiro[pyrrolidine-3,7′-pyrrolo[3,2-c]pyridine]-1-carboxylate (compound 498)

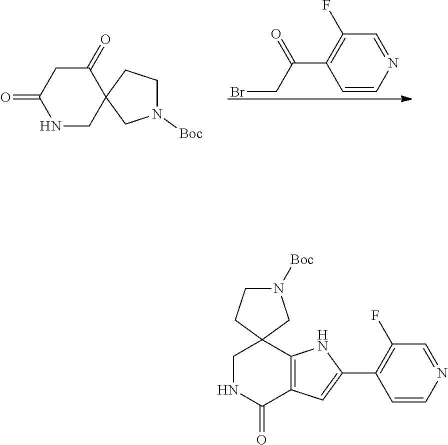 [SMILES: CC(C)(C)OC(=O)N1CCC2(CNC(=O)CC2=O)C1 ; O=C(CBr)c1ccncc1F ; CC(C)(C)OC(=O)N1CCC2(CNC(=O)c3cc(-c4ccncc4F)[nH]c32)C1]

To a stirred mixture of tert-butyl 8,10-dioxo-2,7-diazaspiro[4.5]decane-2-carboxylate (3 g, 11.181 mmol, 1 equiv) and 2-bromo-1-(3-fluoropyridin-4-yl)ethanone (2.44 g, 11.181 mmol, 1 equiv) in EtOH (20 mL) were added NH₄OAc (3.45 g, 44.724 mmol, 4 equiv). The resulting mixture was stirred for 2 h at 50 degrees. The resulting mixture was extracted with EtOAc (3×30 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 100% gradient in 10 min: detector, UV 254 nm. This resulted in tert-butyl 2′-(3-fluoropyridin-4-yl)-4′-oxo-5′,6′-dihydro-1′H-spiro[pyrrolidine-3,7′-pyrrolo[3,2-c]pyridine]-1-carboxylate (3.4 g, 78.69%) as a yellow solid.

LC-MS: M+H found: 387.2.

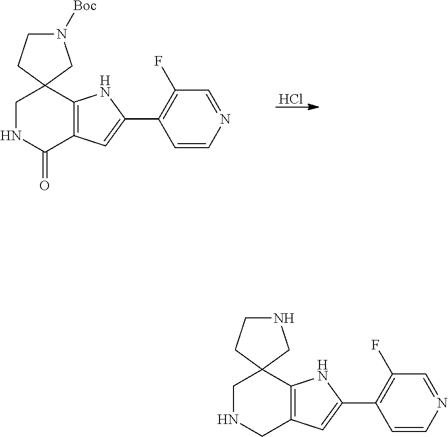 [SMILES: CC(C)(C)OC(=O)N1CCC2(CNC(=O)c3cc(-c4ccncc4F)[nH]c32)C1 ; Cl ; Fc1cnccc1-c1cc2c([nH]1)C1(CCNC1)CNC2]

To a stirred mixture of tert-butyl 2′-(3-fluoropyridin-4-yl)-4′-oxo-5′,6′-dihydro-1′H-spiro[pyrrolidine-3,7′-pyrrolo[3,2-c]pyridine]-1-carboxylate (3 g, 7.763 mmol, 1 equiv) was added HCl (gas) in 1,4-dioxane (50 mL) in portions at room temperature. The final reaction mixture was stirred for 2 h. The resulting mixture was concentrated under reduced pressure. The residue was washed with water (2×10 mL). The resulting mixture was extracted with EtOAc (3×20 mL). The combined organic layers were dried over anhydrous Na₂SO₄. After filtration, the filtrate was concentrated under reduced pressure to afford 2′-(3-fluoropyridin-4-yl)-5′,6′-dihydro-1′H-spiro[pyrrolidine-3,7′-pyrrolo[3,2-c]pyridin]-4′-one (2.5 g, 112.47%) as a yellow solid.

LC-MS: M+H found: 273.1.

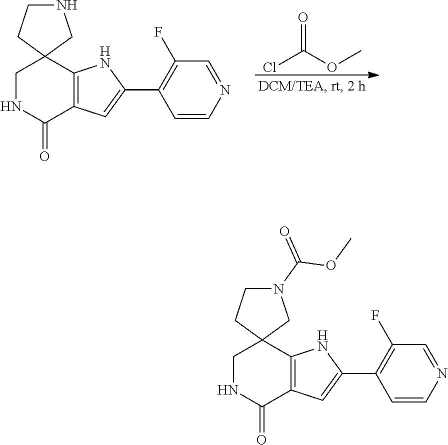

A solution of 2′-(3-fluoropyridin-4-yl)-5′,6′-dihydro-1′H-spiro[pyrrolidine-3,7′-pyrrolo[3,2-c]pyridin]-4′-one (500 mg, 1.746 mmol, 1.00 equiv) in DCM (10 mL) was treated with TEA (530.14 mg, 5.238 mmol, 3 equiv) for 30 min at 0° C. under nitrogen atmosphere followed by the addition of methyl chloroformate (165.01 mg, 1.746 mmol, 1 equiv) in portions at rt. The final reaction mixture was stirred for 1 h. The resulting mixture was concentrated under vacuum. The aqueous layer was extracted with DCM (3×20 mL). The combined organic phase was concentrated under vacuum to afford methyl 2′-(3-fluoropyridin-4-yl)-4′-oxo-5′,6′-dihydro-1′H-spiro[pyrrolidine-3,7′-pyrrolo[3,2-c]pyridine]-1-carboxylate (360 mg, 59.87%) as a yellow solid.

LC-MS: M+H found: 345.1.

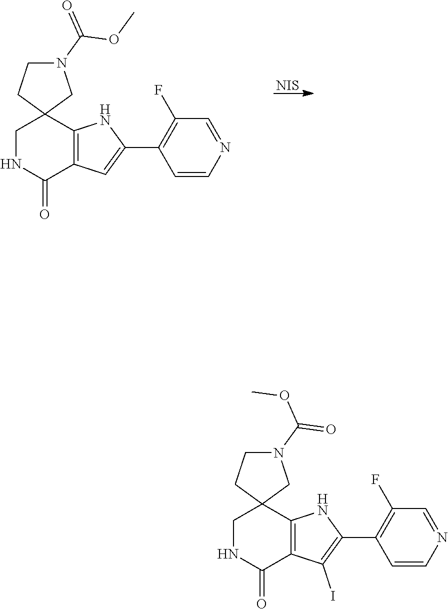 [SMILES: COC(=O)N1CCC2(CNC(=O)c3cc(-c4ccncc4F)[nH]c32)C1 ; "NIS" ; COC(=O)N1CCC2(CNC(=O)c3c2[nH]c(-c2ccncc2F)c3I)C1]

To a stirred solution of methyl 2′-(3-fluoropyridin-4-yl)-4′-oxo-5′,6′-dihydro-1′H-spiro[pyrrolidine-3,7′-pyrrolo[3,2-c]pyridine]-1-carboxylate (350 mg, 1.016 mmol, 1.00 equiv) in DMF (4 mL) was added NIS (274.41 mg, 1.219 mmol, 1.2 equiv) in portions at rt. The final reaction mixture was stirred for 3 h at rt. The resulting mixture was extracted with EA (3×10 mL). The combined organic layers were washed with brine (3×30 mL), dried over anhydrous sodium sulphate. After filtration, the filtrate was concentrated under reduced pressure to afford methyl 2′-(3-fluoropyridin-4-yl)-3′-iodo-4′-oxo-5′,6′-dihydro-1′H-spiro[pyrrolidine-3,7′-pyrrolo[3,2-c]pyridine]-1-carboxylate (335 mg, 70.09%) as a brown yellow solid.

LC-MS: M+H found: 471.0.

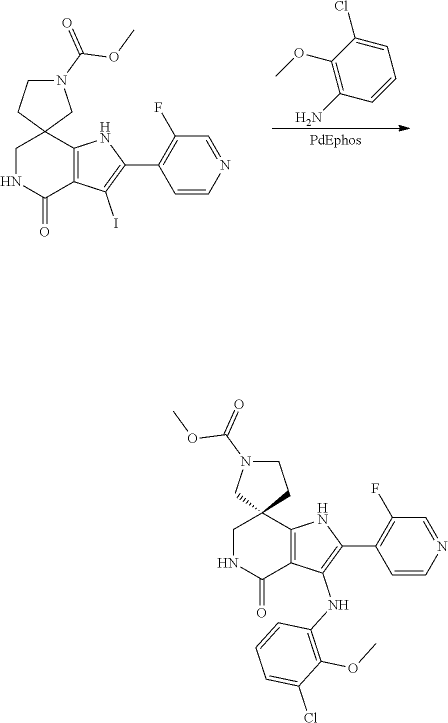

To a stirred solution of methyl 2′-(3-fluoropyridin-4-yl)-3′-iodo-4′-oxo-5′,6′-dihydro-1′H-spiro[pyrrolidine-3,7′-pyrrolo[3,2-c]pyridine]-1-carboxylate (310 mg, 0.659 mmol, 1.00 equiv), EPhos Pd G4 (60.55 mg, 0.066 mmol, 0.1 equiv) and EPhos (70.51 mg, 0.132 mmol, 0.2 equiv) in DMF (4 mL) were added Cs₂CO₃ (429.58 mg, 1.318 mmol, 2 equiv) and 3-chloro-2-methoxyaniline (124.67 mg, 0.791 mmol, 1.2 equiv) under N2 atmosphere. The final reaction mixture was stirred for 5 h at 50 degrees. The precipitated solids were collected by filtration and washed with MeOH (3×1 mL). The crude product was purified by Pre-Chiral HPLC with the following conditions (Column: CHIRALPAK IG, 2*25 cm, 5 μm; Mobile Phase A: Hex:DCM=3:1 (0.5% 2M NH3-MeOH)-HPLC, Mobile Phase B: EtOH-HPLC; Flow rate: 20 mL/min; Gradient: 20% B to 20% B in 23 min; Wave Length: 220/254 nm; RT1 (min): 12.71; RT2 (min): 19.13; Sample Solvent: ETOH:DCM=1:1; Injection Volume: 0.8 mL; Number Of Runs: 6) to afford methyl (3R)-3′-[(3-chloro-2-methoxyphenyl)amino]-2′-(3-fluoropyridin-4-yl)-4′-oxo-5′,6′-dihydro-1′H-spiro[pyrrolidine-3,7′-pyrrolo[3,2-c]pyridine]-1-carboxylate (25.1 mg, 7.57%) as a white solid.

LC-MS: (M+H)⁺ found: 500.0.

¹H NMR (400 MHz, DMSO-d6) δ 11.58 (s, 1H), 8.50 (d, J=2.5 Hz, 1H), 8.35-8.28 (m, 1H), 7.62 (s, 1H), 7.52-7.44 (m, 1H), 7.38 (s, 1H), 6.69-6.54 (m, 2H), 6.11 (dd, J=7.8, 1.8 Hz, 1H), 3.84 (s, 3H), 3.62 (s, 3H), 3.57 (d, J=11.5 Hz, 3H), 3.44 (dd, J=7.0, 5.1 Hz, 3H), 2.40 (s, 1H), 2.08 (s, 1H).

### Example 25. methyl (3S)-3′-[(3-chloro-2-methoxyphenyl)amino]-2′-(3-fluoropyridin-4-yl)-4′-oxo-5′,6′-dihydro-1′H-spiro[pyrrolidine-3,7′-pyrrolo[3,2-c]pyridine]-1-carboxylate (compound 497)

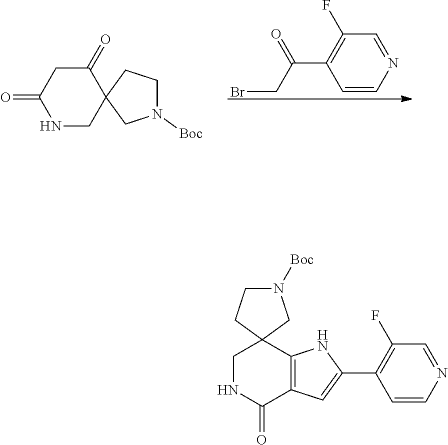 [SMILES: CC(C)(C)OC(=O)N1CCC2(CNC(=O)CC2=O)C1 ; O=C(CBr)c1ccncc1F ; CC(C)(C)OC(=O)N1CCC2(CNC(=O)c3cc(-c4ccncc4F)[nH]c32)C1]

To a stirred mixture of tert-butyl 8,10-dioxo-2,7-diazaspiro[4.5]decane-2-carboxylate (3 g, 11.181 mmol, 1 equiv) and 2-bromo-1-(3-fluoropyridin-4-yl)ethanone (2.44 g, 11.181 mmol, 1 equiv) in EtOH (20 mL) were added NH₄OAc (3.45 g, 44.724 mmol, 4 equiv). The resulting mixture was stirred for 2 h at 50° C. The resulting mixture was extracted with EtOAc (3×30 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 100% gradient in 10 min; detector, UV 254 nm. This resulted in tert-butyl 2′-(3-fluoropyridin-4-yl)-4′-oxo-5′,6′-dihydro-1′H-spiro[pyrrolidine-3,7′-pyrrolo[3,2-c]pyridine]-1-carboxylate (3.4 g, 78.69%) as a yellow solid.

LC-MS: M+H found: 387.2.

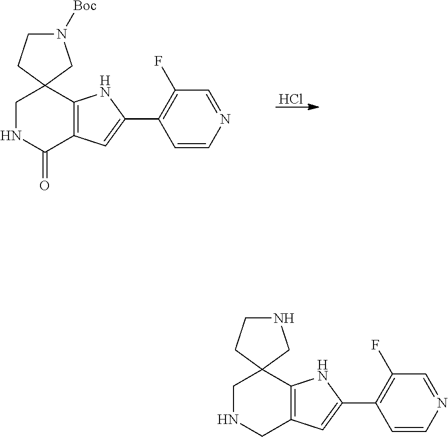 [SMILES: CC(C)(C)OC(=O)N1CCC2(CNC(=O)c3cc(-c4ccncc4F)[nH]c32)C1 ; Cl ; Fc1cnccc1-c1cc2c([nH]1)C1(CCNC1)CNC2]

To a stirred mixture of tert-butyl 2′-(3-fluoropyridin-4-yl)-4′-oxo-5′,6′-dihydro-1′H-spiro[pyrrolidine-3,7′-pyrrolo[3,2-c]pyridine]-1-carboxylate (3 g, 7.763 mmol, 1 equiv) was added HCl (gas) in 1,4-dioxane (50 mL) in portions at room temperature. The final reaction mixture was stirred for 2 h. The resulting mixture was concentrated under reduced pressure. The residue was washed with water (2×10 mL). The resulting mixture was extracted with EtOAc (3×20 mL). The combined organic layers were dried over anhydrous Na₂SO₄. After filtration, the filtrate was concentrated under reduced pressure to afford 2′-(3-fluoropyridin-4-yl)-5′,6′-dihydro-1′H-spiro[pyrrolidine-3,7′-pyrrolo[3,2-c]pyridin]-4′-one (2.5 g, 112.47%) as a yellow solid.

LC-MS: M+H found: 273.1.

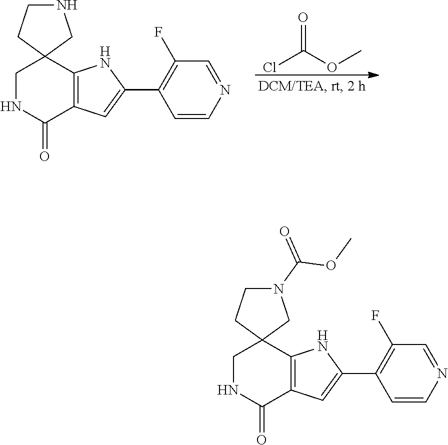

A solution of 2′-(3-fluoropyridin-4-yl)-5′,6′-dihydro-1′H-spiro[pyrrolidine-3,7′-pyrrolo[3,2-c]pyridin]-4′-one (500 mg, 1.746 mmol, 1.00 equiv) in DCM (10 mL) was treated with TEA (530.14 mg, 5.238 mmol, 3 equiv) for 30 min at 0° C. under nitrogen atmosphere followed by the addition of methyl chloroformate (165.01 mg, 1.746 mmol, 1 equiv) in portions at rt. The reaction mixture was stirred for 1 h, then concentrated under vacuum. The aqueous layer was extracted with DCM (3×20 mL). The combined organic phase were concentrated under vacuum to afford methyl 2′-(3-fluoropyridin-4-yl)-4′-oxo-5′,6′-dihydro-1′H-spiro[pyrrolidine-3,7′-pyrrolo[3,2-c]pyridine]-1-carboxylate (360 mg, 59.87%) as a yellow solid.

LC-MS: M+H found: 345.1.

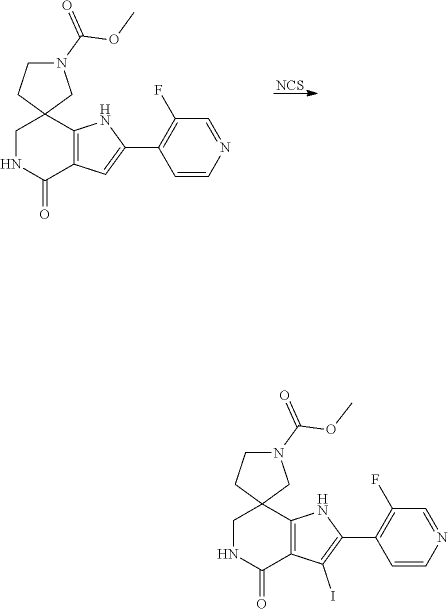 [SMILES: COC(=O)N1CCC2(CNC(=O)c3cc(-c4ccncc4F)[nH]c32)C1 ; "NCS" ; COC(=O)N1CCC2(CNC(=O)c3c2[nH]c(-c2ccncc2F)c3I)C1]

To a stirred solution of methyl 2′-(3-fluoropyridin-4-yl)-4′-oxo-5′,6′-dihydro-1′H-spiro[pyrrolidine-3,7′-pyrrolo[3,2-c]pyridine]-1-carboxylate (350 mg, 1.016 mmol, 1.00 equiv) in DMF (4 mL) was added NIS (274.41 mg, 1.219 mmol, 1.2 equiv) in portions at rt. The final reaction mixture was stirred for 3 h at rt. The resulting mixture was extracted with EA (3×10 mL). The combined organic layers were washed with brine (3×30 mL), dried over anhydrous sodium sulphate. After filtration, the filtrate was concentrated under reduced pressure to afford methyl 2′-(3-fluoropyridin-4-yl)-3′-iodo-4′-oxo-5′,6′-dihydro-1′H-spiro[pyrrolidine-3,7′-pyrrolo[3,2-c]pyridine]-1-carboxylate (335 mg, 70.09%) as a brown yellow solid.

LC-MS: M+H found: 471.0.

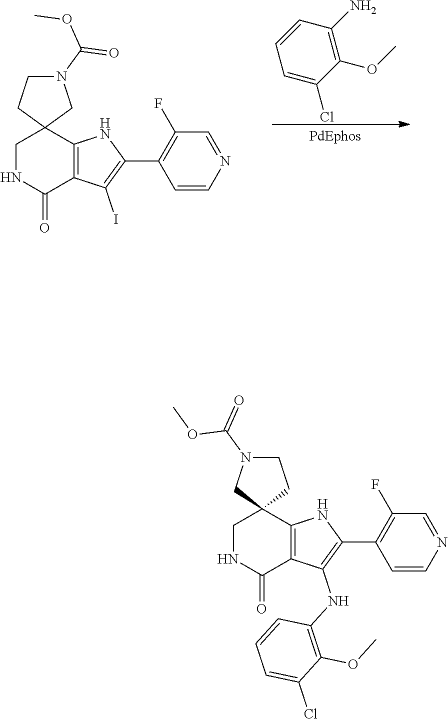

To a stirred solution of methyl 2′-(3-fluoropyridin-4-yl)-3′-iodo-4′-oxo-5′,6′-dihydro-1′H-spiro[pyrrolidine-3,7′-pyrrolo[3,2-c]pyridine]-1-carboxylate (310 mg, 0.659 mmol, 1.00 equiv), EPhos Pd G4 (60.55 mg, 0.066 mmol, 0.1 equiv), and EPhos (70.51 mg, 0.132 mmol, 0.2 equiv) in DMF (4 mL) were added Cs₂CO₃ (429.58 mg, 1.318 mmol, 2 equiv) and 3-chloro-2-methoxyaniline (124.67 mg, 0.791 mmol, 1.2 equiv) under N2 atmosphere. The final reaction mixture was stirred for 5 h at 50 degrees. The precipitated solids were collected by filtration and washed with MeOH (3×1 mL). The crude product was purified by Pre-Chiral HPLC with the following conditions (Column: CHIRALPAK IG, 2*25 cm, 5 μm; Mobile Phase A: Hex:DCM=3:1 (0.5% 2M NH₃-MeOH)-HPLC, Mobile Phase B. EtOH-HPLC; Flow rate: 20 mL/min; Gradient: 20% B to 20% B in 23 min; Wave Length: 220/254 nm; RT1 (min): 12.71; RT2 (min): 19.13; Sample Solvent: ETOH:DCM=1:1; Injection Volume: 0.8 mL; Number Of Runs: 6) to afford methyl (3S)-3′-[(3-chloro-2-methoxyphenyl)amino]-2′-(3-fluoropyridin-4-yl)-4′-oxo-5′,6′-dihydro-1′H-spiro[pyrrolidine-3,7′-pyrrolo[3,2-c]pyridine]-1-carboxylate (25.7 mg, 7.74%) as a white solid.

LC-MS: (M+H)⁺ found: 500.0.

¹H NMR (400 MHz, DMSO-d6) δ 11.58 (s, 1H), 8.50 (d, J=2.5 Hz, 1H), 8.34 (dd, J=5. 1, 1.1 Hz, 1H), 7.62 (s, 1H), 7.48 (t, J=5.8 Hz, 1H), 7.38 (s, 1H), 6.68-6.57 (m, 2H), 6.11 (dd, J=7.8, 1.9 Hz, 1H), 3.84 (s, 3H), 3.62 (s, 3H), 3.57 (d, J=11.6 Hz, 3H), 3.44 (dd, J=7.0, 5.1 Hz, 3H), 2.40 (s, 1H), 2.08 (s, 1H).

### Example 26 3′-[(3-chloro-2-methoxyphenyl)amino]-1-(2,2-difluoroethyl)-2′-(3-fluoropyridin-4-yl)-5′,6′-dihydro-1′H-spiro[azetidine-3,7′-pyrrolo[3,2-c]pyridin]-4′-one (compound 519)

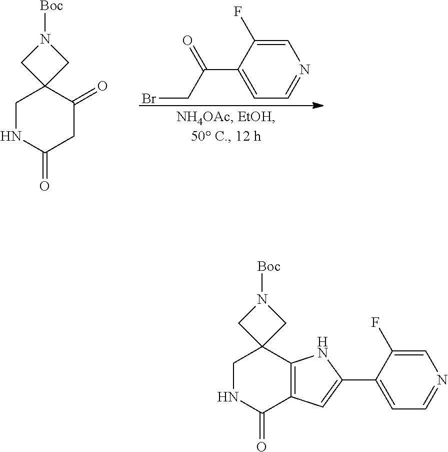 [SMILES: CC(C)(C)OC(=O)N1CC2(CNC(=O)CC2=O)C1 ; O=C(CBr)c1ccncc1F ; CC(C)(C)OC(=O)N1CC2(CNC(=O)c3cc(-c4ccncc4F)[nH]c32)C1]

A mixture of tert-butyl 7,9-dioxo-2,6-diazaspiro[3.5]nonane-2-carboxylate (500 mg, 1.966 mmol, 1.00 equiv) and NH₄OAc (606.27 mg, 7.864 mmol, 4 equiv) in EtOH (10 mL) was stirred for overnight at 50 degrees C. under nitrogen atmosphere. The resulting mixture was concentrated under reduced pressure. The resulting mixture was diluted with water. The precipitated solids were collected by filtration and washed with water. This resulted in tert-butyl 2′-(3-fluoropyridin-4-yl)-4′-oxo-5′,6′-dihydro-1′H-spiro[azetidine-3,7′-pyrrolo[3,2-c]pyridine]-1-carboxylate (470 mg, 64.19%) as a white solid.

LC-MS: M+H found: 373.

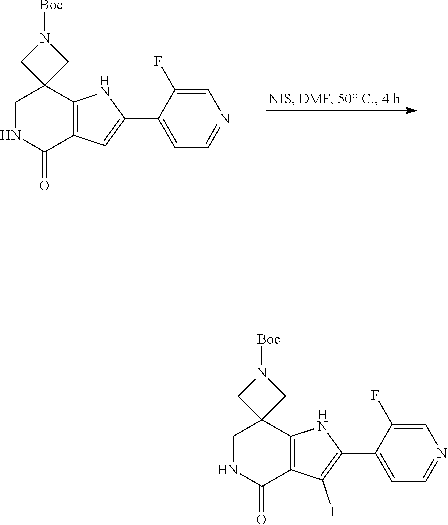 [SMILES: CC(C)(C)OC(=O)N1CC2(CNC(=O)c3cc(-c4ccncc4F)[nH]c32)C1 ; CC(C)(C)OC(=O)N1CC2(CNC(=O)c3c2[nH]c(-c2ccncc2F)c3I)C1]

A mixture of tert-butyl 2′-(3-fluoropyridin-4-yl)-4′-oxo-5′,6′-dihydro-1′H-spiro[azetidine-3,7′-pyrrolo[3,2-c]pyridine]-1-carboxylate (300 mg, 0.806 mmol, 1.00 equiv) and NIS (199.37 mg, 0.887 mmol, 1.1 equiv) in DMF (6 mL) was stirred for overnight at room temperature under nitrogen atmosphere. The resulting mixture was diluted with water. The resulting mixture was extracted with CH2Cl2. The combined organic layers were washed with brine, dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 50% gradient in 30 min; detector, UV 254 nm. This resulted in tert-butyl 2′-(3-fluoropyridin-4-yl)-3′-iodo-4′-oxo-5′,6′-dihydro-1′H-spiro[azetidine-3,7′-pyrrolo[3,2-c]pyridine]-1-carboxylate (300 mg, 74.73%) as a white solid.

LC-MS: (M+H)⁺ found: 499.

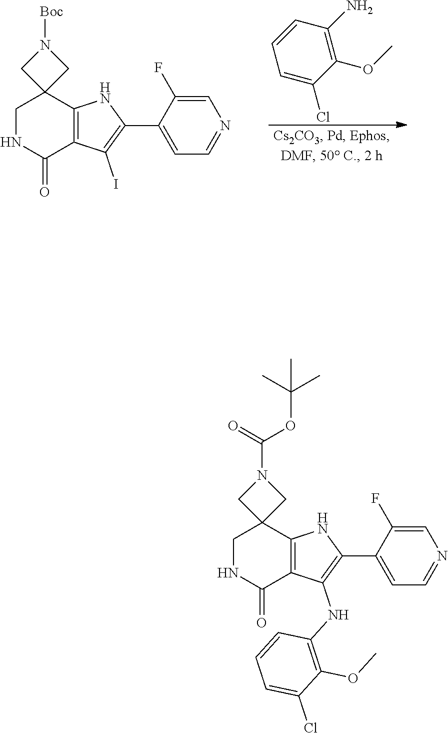

A mixture of tert-butyl 2′-(3-fluoropyridin-4-yl)-3′-iodo-4′-oxo-5′,6′-dihydro-1′H-spiro[azetidine-3,7′-pyrrolo[3,2-c]pyridine]-1-carboxylate (300 mg, 0.602 mmol, 1.00 equiv), 3-chloro-2-methoxyaniline (142.32 mg, 0.903 mmol, 1.5 equiv), EPhos Pd G4 (55.30 mg, 0.060 mmol, 0.1 equiv), EPhos (64.39 mg, 0.120 mmol, 0.2 equiv) and Cs₂CO₃ (588.48 mg, 1.806 mmol, 3 equiv) in DMF (6 mL) was stirred for 1 h at 50 degrees C. under nitrogen atmosphere. The resulting mixture was diluted with water. The resulting mixture was extracted with CH₂Cl₂. The combined organic layers were dried over anhydrous Na₂SO₄. After filtration, the filtrate was concentrated under reduced pressure. The residue was purified by reverse flash chromatography with the following conditions: column, C18 silica gel, mobile phase, MeCN in water, 10% to 50% gradient in 30 min; detector, UV 254 nm. This resulted in tert-butyl 3′-[(3-chloro-2-methoxyphenyl)amino]-2′-(3-fluoropyridin-4-yl)-4′-oxo-5′,6′-dihydro-1′H-spiro[azetidine-3,7′-pyrrolo[3,2-c]pyridine]-1-carboxylate (300 mg, 94.38%) as a white solid.

LC-MS: (M+H)⁺ found: 528.

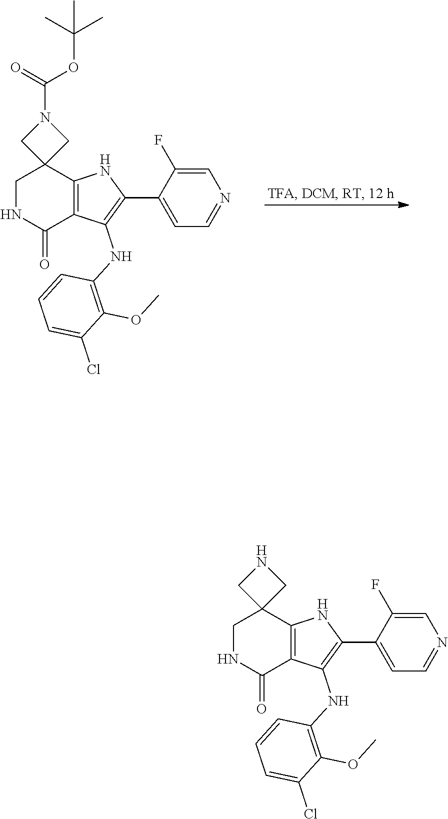 [SMILES: COc1c(Cl)cccc1Nc1c(-c2ccncc2F)[nH]c2c1C(=O)NCC21CN(C(=O)OC(C)(C)C)C1 ; COc1c(Cl)cccc1Nc1c(-c2ccncc2F)[nH]c2c1C(=O)NCC21CNC1]

A mixture of tert-butyl 3′-[(3-chloro-2-methoxyphenyl)amino]-2′-(3-fluoropyridin-4-yl)-4′-oxo-5′,6′-dihydro-1′H-spiro[azetidine-3,7′-pyrrolo[3,2-c]pyridine]-1-carboxylate (300 mg, 0.568 mmol, 1.00 equiv) in HCl (gas) in 1,4-dioxane (6 mL) was stirred for overnight at room temperature under air atmosphere. The resulting mixture was concentrated under reduced pressure. The mixture was basified to pH 8 with saturated NaHCO₃ (aq.). The precipitated solids were collected by filtration and washed with water. The crude product/resulting mixture was used in the next step directly without further purification. This resulted in 3′-[(3-chloro-2-methoxyphenyl)amino]-2′-(3-fluoropyridin-4-yl)-5′,6′-dihydro-1′H-spiro[azetidine-3,7′-pyrrolo[3,2-c]pyridin]-4′-one (200 mg, 82.27%) as a brown solid.

LC-MS: (M+H)⁺ found: 428.

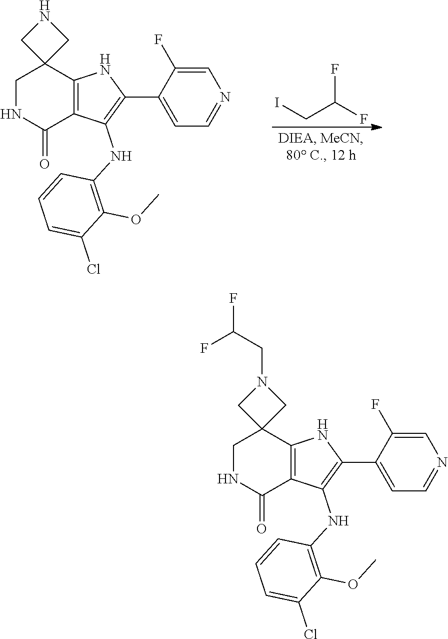 [SMILES: COc1c(Cl)cccc1Nc1c(-c2ccncc2F)[nH]c2c1C(=O)NCC21CNC1 ; FC(F)CI ; COc1c(Cl)cccc1Nc1c(-c2ccncc2F)[nH]c2c1C(=O)NCC21CN(CC(F)F)C1]

A mixture of 3′-[(3-chloro-2-methoxyphenyl)amino]-2′-(3-fluoropyridin-4-yl)-5′,6′-dihydro-1′H-spiro[azetidine-3,7′-pyrrolo[3,2-c]pyridin]-4′-one (200 mg, 0.467 mmol, 1 equiv), 1,1-difluoro-2-iodoethane (107.67 mg, 0.560 mmol, 1.2 equiv), and DIEA (181.24 mg, 1.401 mmol, 3 equiv) in MeCN (4 mL) was stirred for overnight at room temperature under nitrogen atmosphere. The resulting mixture was diluted with water. The resulting mixture was extracted with CH₂Cl₂. The combined organic layers were dried over anhydrous Na₂SO₄. After filtration, the filtrate was concentrated under reduced pressure. The residue was purified by reverse flash chromatography with the following conditions: column, C18 silica gel; mobile phase, MeCN in water, 10% to 50% gradient in 30 min; detector, UV 254 nm. This resulted in 3′-[(3-chloro-2-methoxyphenyl)amino]-1-(2,2-difluoroethyl)-2′-(3-fluoropyridin-4-yl)-5′,6′-dihydro-1′H-spiro[azetidine-3,7′-pyrrolo[3,2-c]pyridin]-4′-one (50 mg, 21.64%) as a light yellow solid.

LC-MS: (M+H)⁺ found: 492.

¹H NMR (300 MHz, Methanol-d4) δ 8.44 (d, J=3.2 Hz, 1H), 8.24 (dd, J=5.3, 0.9 Hz, 1H), 7.54 (dd, J=6.7, 5.2 Hz, 1H), 6.73-6.48 (m, 2H), 6.27-5.77 (m, 2H), 4.03-3.79 (m, 9H), 3.36 (d, J=3.7 Hz, 1H), 3.26 (d, J=3.7 Hz, 1H).

### Example 27. (6S)-3-[(3-chloro-2-methoxyphenyl)amino]-2-{2-methoxypyrido[3,2-d]pyrimidin-8-yl}-6-(trifluoromethyl)-1H,5H,6H,7H-pyrrolo[3,2-c]pyridin-4-one (compound 508)

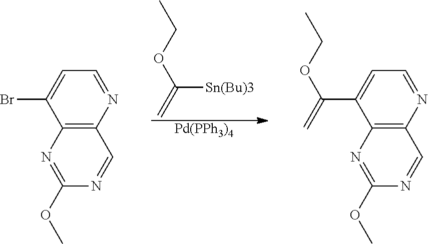

To a stirred solution of tributyl(1-ethoxyethenyl)stannane (12.04 g, 33.326 mmol, 2.0 equiv) and 8-bromo-2-methoxypyrido[3,2-d]pyrimidine (4 g, 16.663 mmol, 1.00 equiv) in 1,4-dioxane (30 mL) was added PPh₃ (2.19 g, 8.332 mmol, 0.5 equiv) in portions at room temperature under nitrogen atmosphere. The resulting mixture was stirred for overnight at 90° C. under nitrogen atmosphere. The resulting mixture was diluted with water. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (5:1) to afford 8-(1-ethoxyethenyl)-2-methoxypyrido[3,2-d]pyrimidine (3.2 g, 83.05%) as a off-white solid.

LC-MS: M+H found: 232.

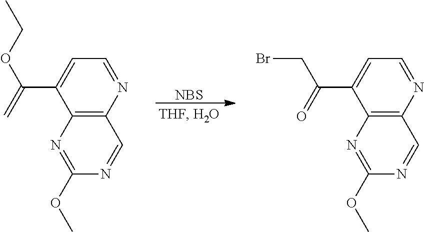

To a stirred solution of 8-(1-ethoxyethenyl)-2-methoxypyrido[3,2-d]pyrimidine (3 g, 12.973 mmol, 1.00 equiv) in THF (25 mL) and H₂O (5 mL) was added NBS (2.31 g, 12.973 mmol, 1.0 equiv) in portions at room temperature under air atmosphere. The resulting mixture was stirred for 1 h at 50° C. under air atmosphere. The resulting mixture was concentrated under reduced pressure. The residue was purified by silica gel column chromatography, eluted with PE/EA (7:1) to afford 2-bromo-1-{2-methoxypyrido[3,2-d]pyrimidin-8-yl}ethanone (2.6 g, 71.05%) as a white solid.

LC-MS: M+H found: 282.

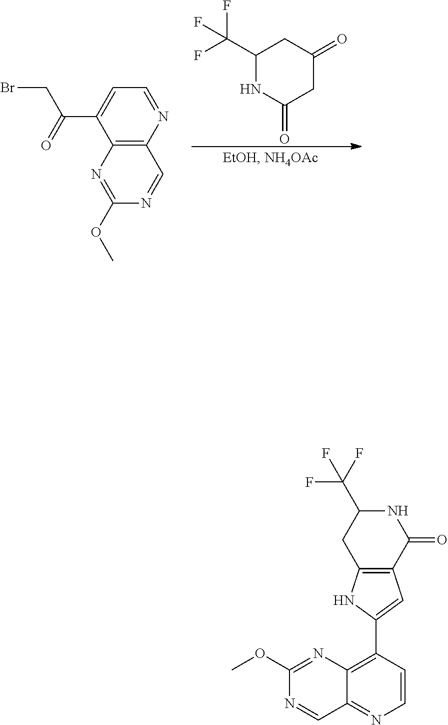 [SMILES: COc1ncc2nccc(C(=O)CBr)c2n1 ; O=C1CC(=O)NC(C(F)(F)F)C1 ; COc1ncc2nccc(-c3cc4c([nH]3)CC(C(F)(F)F)NC4=O)c2n1]

To a stirred solution of 2-bromo-1-{2-methoxypyrido[3,2-d]pyrimidin-8-yl}ethanone (1 g, 3.545 mmol, 1.0 equiv) and 6-(trifluoromethyl)piperidine-2,4-dione (0.71 g, 3.900 mmol, 1.1 equiv) in EtOH (15 mL) was added NH⁴OAc (1.37 g, 17.725 mmol, 5.0 equiv) in portions at room temperature under nitrogen atmosphere. The resulting mixture was stirred for 6 h at 50° C. under nitrogen atmosphere. The resulting mixture was diluted with water. The aqueous layer was extracted with EtOAc (3×30 mL). The residue was purified by silica gel column chromatography, eluted with PE/EA (1:1) to afford 2-{2-methoxypyrido[3,2-d]pyrimidin-8-yl}-6-(trifluoromethyl)-1H,5H,6H,7H-pyrrolo[3,2-c]pyridin-4-one (850 mg, 66.00%) as a light yellow solid.

LC-MS: M+H found: 364.

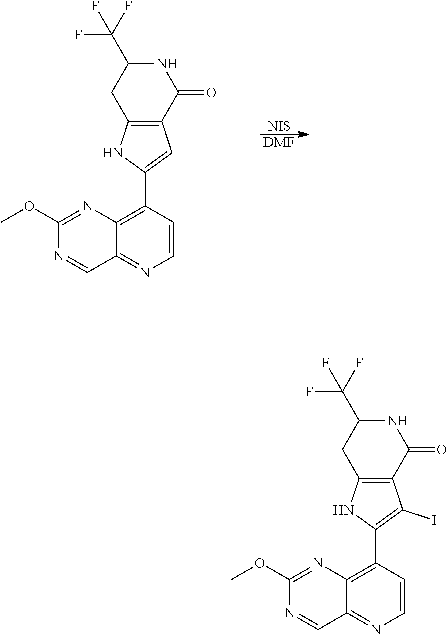

To a stirred solution of 2-{2-methoxypyrido[3,2-d]pyrimidin-8-yl}-6-(trifluoromethyl)-1H,5H,6H,7H-pyrrolo[3,2-c]pyridin-4-one (500 mg, 1.376 mmol, 1.0 equiv) in DMF (10 mL) was added NIS (340.60 mg, 1.514 mmol, 1.1 equiv) in portions at room temperature under air atmosphere. The resulting mixture was stirred for 2 h at 50° C. under air atmosphere. The resulting mixture was diluted with water. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with CH₂Cl₂/MeOH (40:1) to afford 3-iodo-2-{2-methoxypyrido[3,2-d]pyrimidin-8-yl}-6-(trifluoromethyl)-1H,5H,6H,7H-pyrrolo[3,2-c]pyridin-4-one (450 mg, 66.84%) as a light yellow solid.

LC-MS: M+H found: 490.

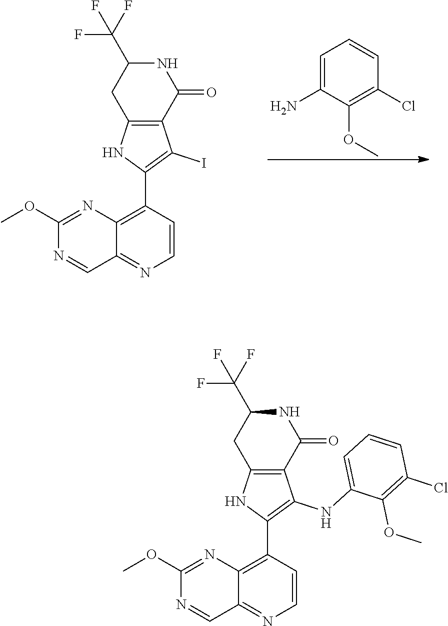

To a stirred solution of 3-iodo-2-{2-methoxypyrido[3,2-d]pyrimidin-8-yl}-6-(trifluoromethyl)-1H,5H,6H,7H-pyrrolo[3,2-c]pyridin-4-one (40 mg, 0.082 mmol, 1.00 equiv) and 3-chloro-2-methoxyaniline (25.77 mg, 0.164 mmol, 2.0 equiv) in DMF (2 mL) were added EPhos (21.86 mg, 0.041 mmol, 0.5 equiv), EPhos Pd G4 (37.55 mg, 0.041 mmol, 0.5 equiv) and Cs₂CO₃ (79.92 mg, 0.246 mmol, 3.0 equiv) at room temperature in portions under nitrogen atmosphere. The resulting mixture was stirred for 1 h at 50° C. under nitrogen atmosphere. The resulting mixture was diluted with water. The resulting mixture was extracted with CH₂Cl₂ (3×20 mL). The combined organic layers were dried over anhydrous Na₂SO₄. After filtration, the filtrate was concentrated under reduced pressure. The crude product (mg) was purified by Prep-HPLC with the following conditions (Column: CHIRALPAK IG-3, 4.6*50 mm, 3 um; Mobile Phase A: Hex:DCM=3:1) (0.1% DEA):EtOH=70:30; Flow rate: 1 mL/min; Gradient: 0% B to 0% B; Injection Volume: 5 ul mL) to afford (6S)-3-[(3-chloro-2-methoxyphenyl)amino]-2-{2-methoxypyrido[3,2-d]pyrimidin-8-yl}-6-(trifluoromethyl)-1H,5H,6H,7H-pyrrolo[3,2-c]pyridin-4-one (8.1 mg, 18.98%) as a white solid.

LC-MS: M+H found: 520.

¹H NMR (300 MHz, DMSO-d6) δ 12.06 (s, 1H), 9.51 (s, 1H), 8.73 (d, J=4.8 Hz, 1H), 7.94 (d, J=4.3 Hz, 1H), 7.76-7.64 (m, 2H), 6.75-6.62 (m, 2H), 6.15 (dd, J=6.9, 2.8 Hz, 1H), 4.46 (s, 1H), 4.21 (s, 3H), 3.86 (s, 3H), 3.34 (s, 1H), 3.24 (s, 1H).

### Example 28. (6R)-3-[(3-chloro-2-methoxyphenyl)amino]-2-{2-methoxypyrido[3,2-d]pyrimidin-8-yl}-6-(trifluoromethyl)-1H,5H,6H,7H-pyrrolo[3,2-c]pyridin-4-one (compound 507)

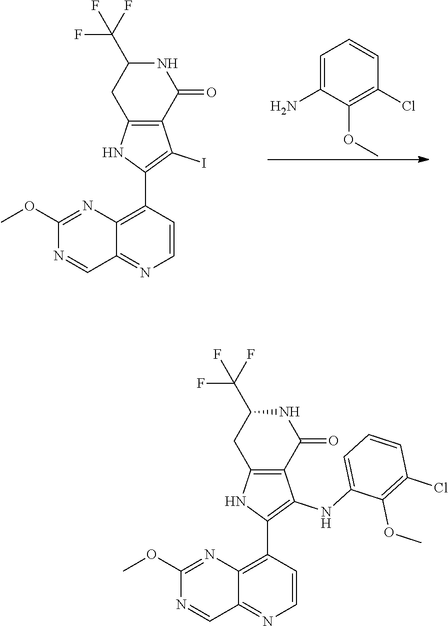

To a stirred solution of 3-iodo-2-{2-methoxypyrido[3,2-d]pyrimidin-8-yl}-6-(trifluoromethyl)-1H,5H,6H,7H-pyrrolo[3,2-c]pyridin-4-one (40 mg, 0.082 mmol, 1.00 equiv) and 3-chloro-2-methoxyaniline (25.77 mg, 0.164 mmol, 2.0 equiv) in DMF (2 mL) were added EPhos (21.86 mg, 0.041 mmol, 0.5 equiv), EPhos Pd G4 (37.55 mg, 0.041 mmol, 0.5 equiv) and Cs₂CO₃ (79.92 mg, 0.246 mmol, 3.0 equiv) at room temperature in portions under nitrogen atmosphere. The resulting mixture was stirred for 1 h at 50° C. under nitrogen atmosphere. The resulting mixture was diluted with water. The resulting mixture was extracted with CH₂Cl2 (3×20 mL). The combined organic layers were dried over anhydrous Na₂SO₄. After filtration, the filtrate was concentrated under reduced pressure. The crude product (mg) was purified by Prep-HPLC with the following conditions (Column: CHIRALPAK IG-3, 4.6*50 mm, 3 um; Mobile Phase A: Hex:DCM=3:1) (0.1% DEA):EtOH=70: 30; Flow rate: 1 mL/min; Gradient: 0% B to 0% B; Injection Volume: 5 ul mL) to afford (6R)-3-[(3-chloro-2-methoxyphenyl)amino]-2-{2-methoxypyrido[3,2-d]pyrimidin-8-yl}-6-(trifluoromethyl)-1H,5H,6H,7H-pyrrolo[3,2-c]pyridin-4-one (13.6 mg, 25.44%) as a yellow solid.

LC-MS: M+H found: 520.

¹H NMR (300 MHz, DMSO-d6) δ 12.06 (s, 1H), 9.51 (s, 1H), 8.73 (d, J=4.8 Hz, 1H), 7.94 (d, J=4.3 Hz, 1H), 7.76-7.64 (m, 2H), 6.75-6.62 (m, 2H), 6.15 (dd, J=6.9, 2.8 Hz, 1H), 4.46 (s, 1H), 4.21 (s, 3H), 3.86 (s, 3H), 3.34 (s, 1H), 3.24 (s, 1H).

### Example 29. 3′-[(3-bromo-2-methoxyphenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 561)

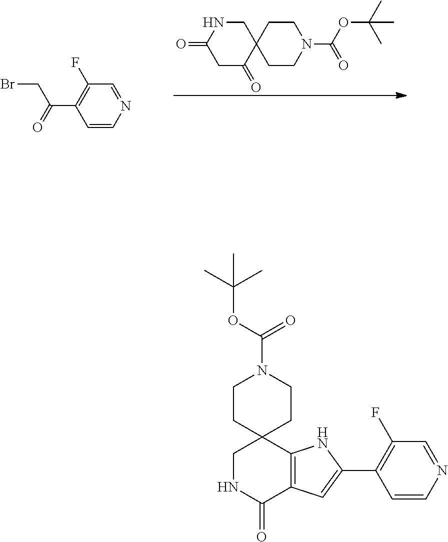 [SMILES: O=C(CBr)c1ccncc1F ; CC(C)(C)OC(=O)N1CCC2(CC1)CNC(=O)CC2=O ; CC(C)(C)OC(=O)N1CCC2(CC1)CNC(=O)c1cc(-c3ccncc3F)[nH]c12]

To a stirred solution of 2-bromo-1-(3-fluoropyridin-4-yl)ethanone (5 g, 22.933 mmol, 1 equiv) and tert-butyl 3,5-dioxo-2,9-diazaspiro[5.5]undecane-9-carboxylate (6.47 g, 22.933 mmol, 1 equiv) in EtOH (50 mL) was added NH₄OAc (10.61 g, 137.598 mmol, 6 equiv) in portions at 50° C. under N₂ atmosphere. The final reaction mixture was stirred for 2 h. The resulting mixture was concentrated under vacuum. The aqueous layer was extracted with EA (3×20 mL) to afford tert-butyl 2′-(3-fluoropyridin-4-yl)-4′-oxo-5′,6′-dihydro-1′H-spiro[piperidine-4,7′-pyrrolo[3,2-c]pyridine]-1-carboxylate (7 g, 76.22%) as a yellow solid.

LC-MS: M+H found: 401.4.

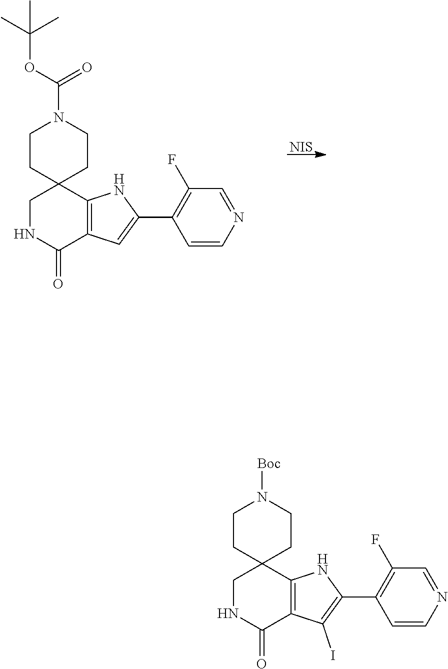 [SMILES: CC(C)(C)OC(=O)N1CCC2(CC1)CNC(=O)c1cc(-c3ccncc3F)[nH]c12 ; "NIS" ; CC(C)(C)OC(=O)N1CCC2(CC1)CNC(=O)c1c2[nH]c(-c2ccncc2F)c1I]

To a stirred solution of isopropyl 2′-(3-fluoropyridin-4-yl)-4′-oxo-5′,6′-dihydro-1′H-spiro[piperidine-4,7′-pyrrolo[3,2-c]pyridine]-1-carboxylate (5 g, 12.939 mmol, 1 equiv) in DMF (50 mL) was added NIS (3.49 g, 15.527 mmol, 1.2 equiv) in portions at RT. The final reaction mixture was stirred for 2 h. The resulting mixture was extracted with EA (3×50 mL). The combined organic layers were washed with brine (3×150 mL), dried over anhydrous sodium sulphate. After filtration, the filtrate was concentrated under reduced pressure to afford tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (6 g, 88.10%) as a yellow solid.

LC-MS: M+H found: 527.1.

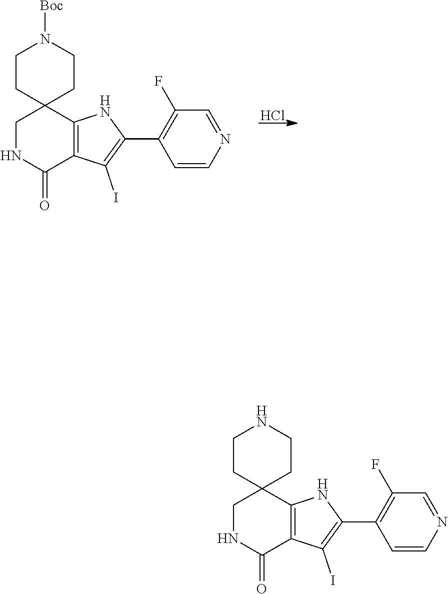 [SMILES: CC(C)(C)OC(=O)N1CCC2(CC1)CNC(=O)c1c2[nH]c(-c2ccncc2F)c1I ; Cl ; O=C1NCC2(CCNCC2)c2[nH]c(-c3ccncc3F)c(I)c21]

To a stirred solution of tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (5 g, 1 equiv) in reaction vessel was added HCl (gas) in 1,4-dioxane (50 mL) in portions at rt under N₂ atmosphere. The final reaction mixture was stirred for 2 h. The resulting mixture was concentrated under vacuum. The residue was acidified to pH=7 with NaHCO₃. The resulting mixture was filtered, the filter cake was washed with MeOH (3×25 mL). The filtrate was concentrated under reduced pressure to afford 2′-(3-fluoropyridin-4-yl)-3′-iodo-1′,4′,5′,6′-tetrahydrospiro[piperidine-4,7′-pyrrolo[3,2-c]pyridine] (3 g, 75.38%) as a yellow solid.

LC-MS: M+H found: 426.9.

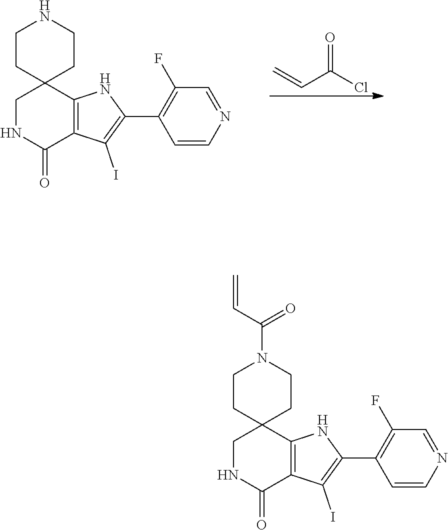

A solution of 2′-(3-fluoropyridin-4-yl)-3′-iodo-5′,6′-dihydro-1′H-spiro[piperidine-4,7′-pyrrolo[3,2-c]pyridin]-4′-one (1.6 g, 3.754 mmol, 1 equiv) in DCM (20 mL) was treated with TEA (1.14 g, 11.262 mmol, 3 equiv) for 30 min at 0° C. under nitrogen atmosphere followed by the addition of acryloyl chloride (509.64 mg, 5.631 mmol, 1.5 equiv) in portions at rt. The final reaction mixture was stirred for 1 h. The reaction was quenched with isopropanol at rt. The resulting mixture was concentrated under vacuum. The residue was purified by silica gel column chromatography, eluted with DCM:EtOH (10:1) to afford 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (1.63 g, 90.56%) as a yellow solid.

LC-MS: M+H found: 481.2.

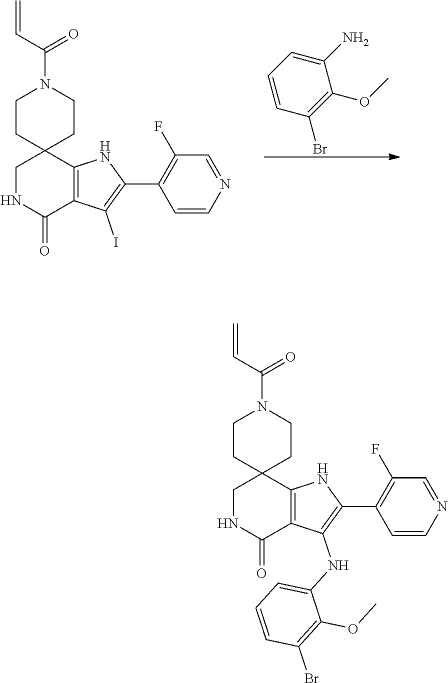

To a stirred solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (1 g, 2.082 mmol, 1 equiv) and 3-bromo-2-methoxyaniline (546.90 mg, 2.707 mmol, 1.3 equiv) in DMF (15 mL) were added EPhos Pd G4 (382.50 mg, 0.416 mmol, 0.2 equiv), EPhos (222.70 mg, 0.416 mmol, 0.2 equiv) and Cs₂CO₃ (1.36 g, 4.164 mmol, 2 equiv) under N₂ atmosphere. The final reaction mixture was stirred for 2 h at 50 degrees. The resulting mixture was filtered, the filter cake was washed with EA (3×5 mL). The filtrate was concentrated under reduced pressure. The residue was purified by Prep-TLC (DCM:MeOH=12:1) to afford crude product. The crude product (130 mg) was purified by Prep-HPLC with the following conditions (Column: XBridge Prep OBD C18 Column, 30*150 mm, 5 μm; Mobile Phase A: Water (10 mmol/L NH₄HCO3+0.1% NH₃*H₂O), Mobile Phase B: ACN; Flow rate: 60 mL/min; Gradient: 24% B to 54% B in 8 min, 54% B; Wave Length: 254: 220 nm: RT1 (min): 7.17) to afford 3′-[(3-bromo-2-methoxyphenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (24.4 mg, 2.11%) as a white solid.

LC-MS: (M+H)⁺ found: 554.3.

¹H NMR (300 MHz, DMSO-d6) δ 11.43 (s, 1H), 8.47 (d, J=2.6 Hz, 1H), 8.31 (dd, J=5.0, 1.2 Hz, 1H), 7.68 (s, 1H), 7.44 (dd, J=6.7, 5.1 Hz, 1H), 7.34 (s, 1H), 6.93-6.74 (m, 2H), 6.55 (t, J=8.1 Hz, 1H), 6.20-6.08 (m, 2H), 5.71 (dd, J=10.4, 2.5 Hz, 1H), 4.42 (d, J=13.2 Hz, 1H), 4.05 (d, J=14.0 Hz, 1H), 3.83 (s, 3H), 3.52 (s, 2H), 3.32-3.25 (m, 1H), 2.85 (t, J=12.6 Hz, 1H), 1.97 (s, 2H), 1.76 (s, 2H).

### Example 30. 3′-{[3-fluoro-2-(methylsulfanyl)phenyl]amino}-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 560)

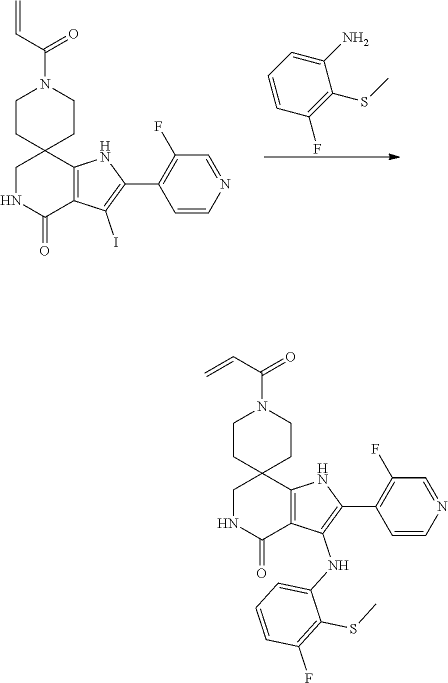

To a solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (500 mg, 1.041 mmol, 1 equiv) and 3-fluoro-2-(methylsulfanyl) aniline (196.40 mg, 1.249 mmol, 1.2 equiv) in DMF (10 mL) were added Cs₂CO₃ (678.39 mg, 2.082 mmol, 2 equiv) and EPhos Pd G4 (191.25 mg, 0.208 mmol, 0.2 equiv) and EPhos (111.35 mg, 0.208 mmol, 0.2 equiv). After stirring for 3 h at 50° C. under a nitrogen atmosphere, the resulting mixture was concentrated under reduced pressure. The aqueous layer was extracted with EA (6×100 mL). The residue was purified by reverse flash chromatography with the following conditions: column, silica gel; mobile phase, MeCN in water; detector, UV 254 nm to afford 3′-{[3-fluoro-2-(methylsulfanyl)phenyl]amino}-2′-(3-fluoropyridin-4-yl)-1-(prop-2-enoyl)-5′,6′-dihydro-1′H-spiro[piperidine-4,7′-pyrrolo[3,2-c]pyridin]-4′-one (8.3 mg, 1.56%) as a light yellow solid.

LC-MS: M+H found: 510.

¹H NMR (400 MHz, DMSO-d6) δ 11.48 (s, 1H), 8.47 (d, J=2.5 Hz, 1H), 8.32 (dd, J=5.0, 1.1 Hz, 1H), 8.15 (s, 1H), 7.43 (dd, J=6.7, 5.0 Hz, 1H), 7.34 (s, 1H), 6.93-6.80 (m, 2H), 6.50-6.42 (m, 1H), 6.15 (dd, J=16.7, 2.5 Hz, 1H), 6.02 (d, J=8.4 Hz, 1H), 5.72 (dd, J=10.4, 2.5 Hz, 1H), 4.43 (d, J=13.3 Hz, 1H), 4.14-4.02 (m, 1H), 3.53 (s, 2H), 3.17 (d, J=5.2 Hz, 1H), 2.87 (dd, J=23.4, 10.3 Hz, 1H), 2.34 (s, 3H), 1.98 (q, J=12.6 Hz, 2H), 1.76 (t, J=12.1 Hz, 2H).

### Example 31. 3′-[(3-chloro-5-fluoro-2-methoxyphenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 559)

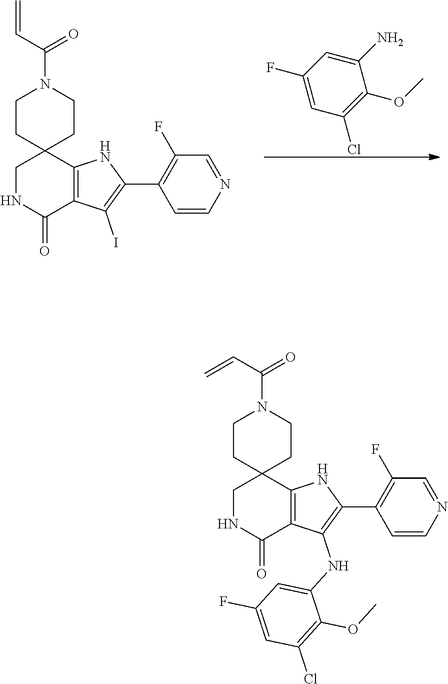 [SMILES: C=CC(=O)N1CCC2(CC1)CNC(=O)c1c2[nH]c(-c2ccncc2F)c1I ; COc1c(N)cc(F)cc1Cl ; C=CC(=O)N1CCC2(CC1)CNC(=O)c1c2[nH]c(-c2ccncc2F)c1Nc1cc(F)cc(Cl)c1OC]

To a stirred solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (500 mg, 1.041 mmol, 1 equiv) and 3-chloro-5-fluoro-2-methoxyaniline (219.36 mg, 1.249 mmol, 1.2 equiv) in DMF (6 mL) were added EPhos Pd G4 (191.25 mg, 0.208 mmol, 0.2 equiv), EPhos (111.35 mg, 0.208 mmol, 0.2 equiv) and Cs₂CO₃ (678.39 mg, 2.082 mmol, 2 equiv) under N2 atmosphere. The final reaction mixture was stirred for 2 h at 50 degrees. The resulting mixture was filtered, the filter cake was washed with EA (3×5 mL). The filtrate was concentrated under reduced pressure. The residue was purified by Prep-TLC (DCM:MeOH=15.1) to afford crude product. The crude product (100 mg) was purified by Prep-HPLC with the following conditions (Column: XBridge Prep OBD C18 Column, 30*150 mm, 5 μm; Mobile Phase A: Water (10 mmol/L NH₄HCO₃+0.1% NH₃*H₂O), Mobile Phase B: ACN; Flow rate: 60 mL/min; Gradient: 24% B to 54% B in 8 min, 54% B; Wave Length: 254; 220 nm: RT1 (min): 7.32) to afford 3′-[(3-chloro-5-fluoro-2-methoxyphenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (21.9 mg, 3.95%) as a white solid.

LC-MS: (M+H)⁺ found: 528.2.

¹H NMR (300 MHz, DMSO-d6) δ 11.53 (s, 1H), 8.52 (d, J=2.5 Hz, 1H), 8.37 (dd, J=5.1, 1.2 Hz, 1H), 7.79 (d, J=1.5 Hz, 1H), 7.49 (dd, J=6.6, 5.0 Hz, 1H), 7.32 (s, 1H), 6.87 (dd, J=16.6, 10.4 Hz, 1H), 6.56-6.47 (m, 1H), 6.15 (dd, J=16.6, 2.5 Hz, 1H), 5.81 (dd, J=11.1, 3.0 Hz, 1H), 5.71 (dd, J=10.4, 2.5 Hz, 1H), 4.42 (d, J=13.2 Hz, 1H), 4.05 (d, J=13.9 Hz, 1H), 3.79 (s, 3H), 3.53 (s, 2H), 3.32-3.25 (m, 1H), 2.85 (t, J=12.7 Hz, 1H), 1.95 (s, 2H), 1.77 (s, 2H).

### Example 32. 3′-[(3-chloro-2-ethylphenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 558)

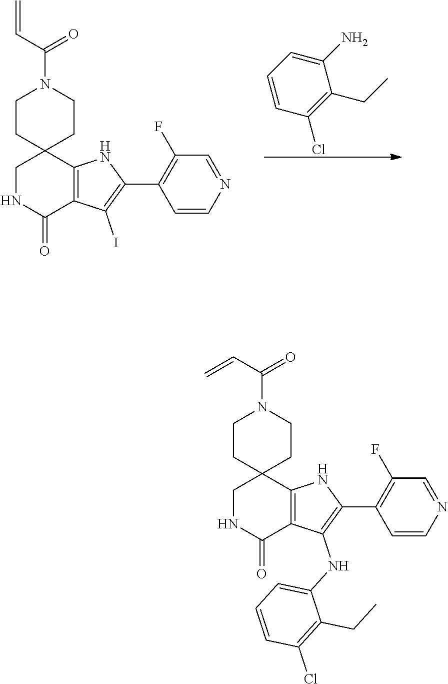

To a solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (300 mg, 0.625 mmol, 1 equiv) and 3-chloro-2-ethylaniline (116.65 mg, 0.750 mmol, 1.2 equiv) in DMF (10 mL) were added Cs₂CO₃ (407.04 mg, 1.250 mmol, 2 equiv) and EPhos Pd G4 (114.75 mg, 0.125 mmol, 0.2 equiv), EPhos (66.81 mg, 0.125 mmol, 0.2 equiv). After stirring for 5 h at 50° C. under a nitrogen atmosphere, the resulting mixture was concentrated under reduced pressure. The aqueous layer was extracted with EA (6×50 mL). The residue was purified by reverse flash chromatography with the following conditions: column, silica gel; mobile phase, MeCN in water; detector, UV 254 nm to afford 3′-[(3-chloro-2-ethylphenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (10.8 mg, 3.32%) as a light yellow solid.

LC-MS: M+H found: 508.

¹H NMR (400 MHz, DMSO-d6) δ 11.39 (s, 1H), 8.43 (d, J=2.6 Hz, 1H), 8.26 (d, J=5.0 Hz, 1H), 7.62 (s, 1H), 7.40-7.31 (m, 2H), 6.88 (dd, J=16.7, 10.4 Hz, 1H), 6.74-6.63 (m, 2H), 6.18 (dd, J=7.2, 2.3 Hz, 1H), 6.13 (d, J=2.5 Hz, 1H), 5.72 (dd, J=10.4, 2.5 Hz, 1H), 4.43 (d, J=13.2 Hz, 1H), 4.06 (d, J=14.0 Hz, 1H), 3.54 (t, J=2.7 Hz, 4H), 2.82 (q, J=7.4 Hz, 2H), 1.98 (d, J=12.6 Hz, 2H), 1.81-1.71 (m, 2H), 1.21 (q, J=7.9 Hz, 3H).

### Example 33. 3′-[(2-ethyl-3-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 557)

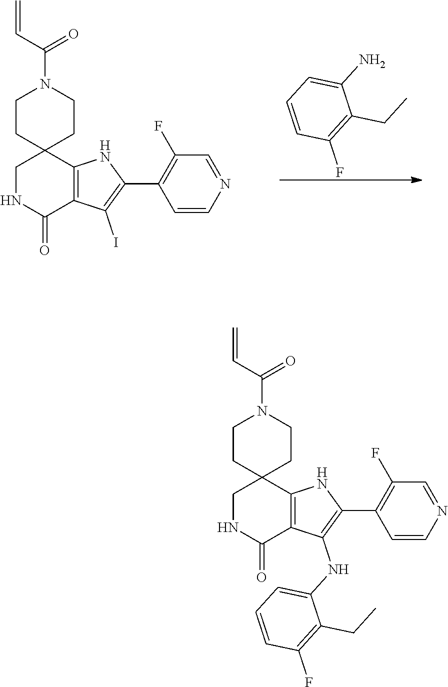

To a stirred solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (100 mg, 0.208 mmol, 1 equiv) and 2-ethyl-3-fluoroaniline (34.77 mg, 0.250 mmol, 1.2 equiv) in DMF (1.5 mL) were added EPhos Pd G4 (38.25 mg, 0.042 mmol, 0.2 equiv), EPhos (22.27 mg, 0.042 mmol, 0.2 equiv) and Cs₂CO₃ (135.68 mg, 0.416 mmol, 2 equiv) under N2 atmosphere. The final reaction mixture was stirred for 2 h at 50 degrees. The resulting mixture was filtered, the filter cake was washed with MeOH. The filtrate was concentrated under reduced pressure. The crude product was purified by reverse phase flash with the following conditions (MeCN:H₂O=47:53) to afford 3′-[(2-ethyl-3-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (8.7 mg, 8.08%) as a white solid.

LC-MS: (M+H)⁺ found: 492.0.

¹H NMR (300 MHz, DMSO-d6) δ 11.35 (s, 1H), 8.41 (d, J=2.6 Hz, 1H), 8.24 (d, J=5.1 Hz, 1H), 7.59 (s, 1H), 7.35 (dd, J=6.8, 4.7 Hz, 2H), 6.94-6.82 (m, 1H), 6.66 (q, J=7.8 Hz, 1H), 6.42 (t, J=8.9 Hz, 1H), 6.14 (dd, J=16.7, 2.6 Hz, 1H), 6.04 (d, J=8.2 Hz, 1H), 5.71 (dd, J=10.4, 2.6 Hz, 1H), 4.42 (d, J=13.1 Hz, 1H), 4.05 (d, J=14.2 Hz, 1H), 3.53 (s, 2H), 3.32-3.25 (m, 1H), 2.85 (t, J=12.9 Hz, 1H), 2.67 (d, J=8.0 Hz, 2H), 2.01 (d, J=19.0 Hz, 2H), 1.76 (s, 2H), 1.18 (q, J=7.2 Hz, 3H).

### Example 34. 1-(but-2-ynoyl)-3′-[(3-chloro-2-methoxyphenyl)amino]-2′-(3-fluoropyridin-4-yl)-5′,6′-dihydro-1′H-spiro[piperidine-4,7′-pyrrolo[3,2-c]pyridin]-4′-one (compound 555)

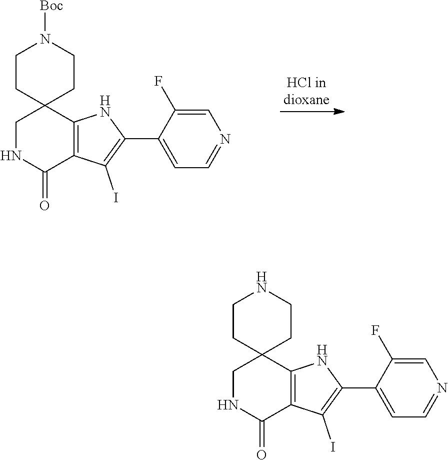 [SMILES: CC(C)(C)OC(=O)N1CCC2(CC1)CNC(=O)c1c2[nH]c(-c2ccncc2F)c1I ; O=C1NCC2(CCNCC2)c2[nH]c(-c3ccncc3F)c(I)c21]

Into a 5 mL round-bottom flask were added tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (4.9 g, 9.309 mmol, 1 equiv) and HCl (gas) in 1,4-dioxane (6.12 mL, 201.540 mmol, 21.65 equiv) at room temperature. The resulting mixture was stirred for 1 h at room temperature. The resulting mixture was concentrated under reduced pressure to afford 2′-(3-fluoropyridin-4-yl)-3′-iodo-5′,6′-dihydro-1′H-spiro[piperidine-4,7′-pyrrolo[3,2-c]pyridin]-4′-one (3.9 g, 98.29%) as a light yellow solid.

LC-MS: M+H found: 427.

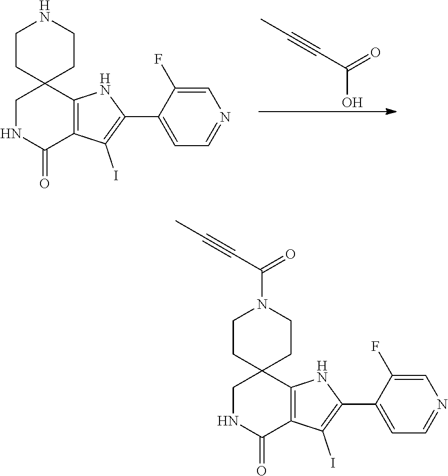

To a stirred solution of 2′-(3-fluoropyridin-4-yl)-3′-iodo-5′,6′-dihydro-1′H-spiro[piperidine-4,7′-pyrrolo[3,2-c]pyridin]-4′-one (200 mg, 0.469 mmol, 1 equiv) and 2-butynoic acid (43.39 mg, 0.516 mmol, 1.1 equiv) in Pyridine (4 mL) were added HOBT (76.08 mg, 0.563 mmol, 1.2 equiv) and EDC*HCl (107.94 mg, 0.563 mmol, 1.2 equiv) at room temperature. The resulting mixture was stirred for 2 h at room temperature. The reaction was quenched with Water. The resulting mixture was extracted with EtOAc. The combined organic layers were washed with brine, dried over anhydrous Na₂SO₄. After filtration, the filtrate was concentrated under reduced pressure. The residue was purified by Prep-TLC (CH₂Cl₂/MeOH 20:1) to afford 1-(but-2-ynoyl)-2′-(3-fluoropyridin-4-yl)-3′-iodo-5′,6′-dihydro-1′H-spiro[piperidine-4,7′-pyrrolo[3,2-c]pyridin]-4′-one (160 mg, 69.27%) as a light yellow solid.

LC-MS: (M+H)⁺ found: 493.

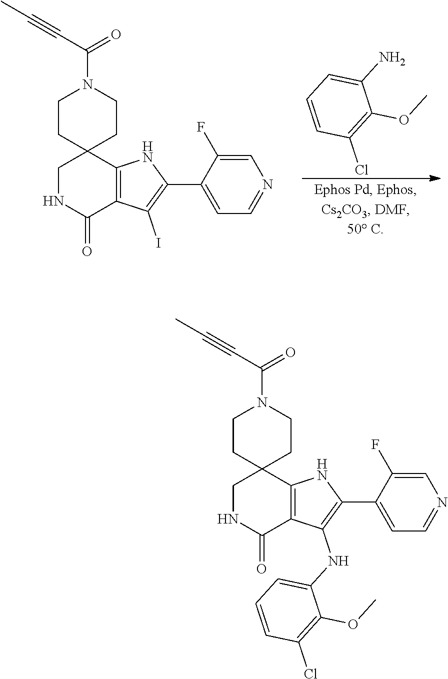

To a stirred solution of 1-(but-2-ynoyl)-2′-(3-fluoropyridin-4-yl)-3′-iodo-5′,6′-dihydro-1′H-spiro[piperidine-4,7′-pyrrolo[3,2-c]pyridin]-4′-one (150 mg, 0.305 mmol, 1 equiv) and 3-chloro-2-methoxyaniline (144.06 mg, 0.915 mmol, 3 equiv) in DMF (3 mL) were added EPhos Pd G4 (55.98 mg, 0.061 mmol, 0.2 equiv) and EPhos (32.59 mg, 0.061 mmol, 0.2 equiv) at room temperature under nitrogen atmosphere. The resulting mixture was stirred for 1 h at 50° C. under nitrogen atmosphere. The reaction was quenched with Water. The resulting mixture was extracted with EtOAc. The combined organic layers were washed with brine, dried over anhydrous Na₂SO₄. After filtration, the filtrate was concentrated under reduced pressure. The crude product (mg) was purified by Prep-HPLC with the following conditions to afford 1-(but-2-ynoyl)-3′-[(3-chloro-2-methoxyphenyl)amino]-2′-(3-fluoropyridin-4-yl)-5′,6′-dihydro-1′H-spiro[piperidine-4,7′-pyrrolo[3,2-c]pyridin]-4′-one as a light yellow solid.

LC-MS: (M+H)⁺ found: 522.

¹H NMR 11.42 (s, 1H), 8.48 (d, 1H), 8.32 (d, 1H), 7.69 (s, 1H), 7.43-7.47 (dd, 1H, J1=5.4 Hz, J2=6.6 Hz), 7.35 (s, 1H), 6.58-6.67 (m, 2H), 6.07-6.10 (dd, 1H, J1=1.8 Hz, J2=7.5 Hz), 4.18-4.32 (m, 2H), 3.84 (s, 3H), 3.51 (s, 2H), 2.84-2.92 (t, 1H, J=18H), 2.04 (s, 3H), 1.76-1.82 (m, 3H), 1.24 (s, 1H), 0.83-0.85 (d, 1H, J=6H).

### Example 35. 3′-[(3-chloro-2-methoxyphenyl)amino]-1-[4-(dimethylamino)but-2-enoyl]-2′-(3-fluoropyridin-4-yl)-5′,6′-dihydro-1′H-spiro[piperidine-4,7′-pyrrolo[3,2-c]pyridin]-4′-one (compound 522)

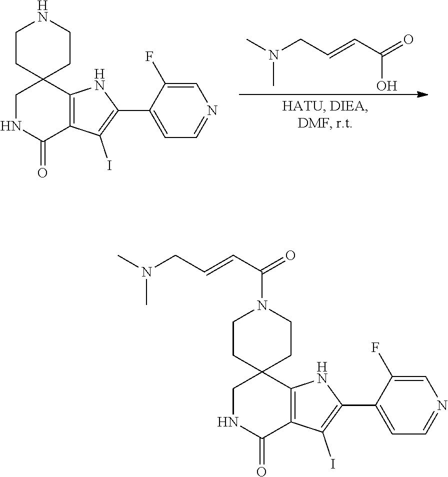

To a stirred solution of 2′-(3-fluoropyridin-4-yl)-3′-iodo-5′,6′-dihydro-1′H-spiro[piperidine-4,7′-pyrrolo[3,2-c]pyridin]-4′-one (200 mg, 0.469 mmol, 1 equiv) and (2E)-4-(dimethylamino)but-2-enoic acid (66.67 mg, 0.516 mmol, 1.1 equiv) in DMF (5 mL, 64.609 mmol, 137.69 equiv) were added HATU (214.10 mg, 0.563 mmol, 1.2 equiv) and DIEA (90.97 mg, 0.704 mmol, 1.5 equiv) at room temperature. The resulting mixture was stirred for 2 h at room temperature. The reaction was quenched with Water. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with CH₂Cl₂/MeOH (30:1) to afford 1-[(2E)-4-(dimethylamino)but-2-enoyl]-2′-(3-fluoropyridin-4-yl)-3′-iodo-5′,6′-dihydro-1′H-spiro[piperidine-4,7′-pyrrolo[3,2-c]pyridin]-4′-one (210 mg, 83.28%) as a light yellow solid.

LC-MS: M+H found: 538.

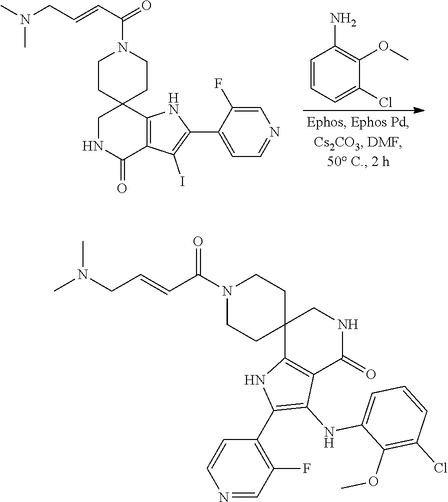

To a stirred solution of 1-[4-(dimethylamino)but-2-enoyl]-2′-(3-fluoropyridin-4-yl)-3′-iodo-5′,6′-dihydro-1′H-spiro[piperidine-4,7′-pyrrolo[3,2-c]pyridin]-4′-one (200 mg, 0.372 mmol, 1 equiv) and 3-chloro-2-methoxyaniline (175.97 mg, 1.116 mmol, 3 equiv) in DMF (5 mL) were added EPhos Pd G4 (68.37 mg, 0.074 mmol, 0.2 equiv) and Cs₂CO₃ (363.79 mg, 1.116 mmol, 3 equiv) at room temperature under nitrogen atmosphere. The resulting mixture was stirred for 2 h at 50° C. under nitrogen atmosphere. The reaction was quenched with water. The resulting mixture was extracted with EtOAc. The combined organic layers were washed with brine, dried over anhydrous Na₂SO₄. After filtration, the filtrate was concentrated under reduced pressure. The crude product was purified by Prep-HPLC with the following conditions (Column: YMC-Actus Triart C18, 30*150 mm, 5 μm; Mobile Phase A: Water (10 mmol/L NH₄HCO₃+0.1% NH₃*H₂O), Mobile Phase B: ACN; Flow rate: 60 mL/min; Gradient: 35% B to 45% B in 8 min, 45% B; Wave Length: 254; 220 nm; RT1 (min): 7.82) to afford 3′-[(3-chloro-2-methoxyphenyl)amino]-1-[4-(dimethylamino)but-2-enoyl]-2′-(3-fluoropyridin-4-yl)-5′,6′-dihydro-1′H-spiro[piperidine-4,7′-pyrrolo[3,2-c]pyridin]-4′-one (15.9 mg, 8.95%) as a light yellow solid.

LC-MS: (M+H)⁺ found 567.

¹H NMR 11.43 (s, 1H), 8.48 (d, 1H, J=2.4 Hz), 8.32 (d, 1H, J=4.8 Hz), 7.69 (s, 1H), 7.42-7.46 (dd, 1H, J1=5.1 Hz, J2=6.6 Hz), 7.34 (s, 1H), 6.60-6.66 (m, 4H), 6.07-6.10 (dd, 1H, J1=1.8 Hz, J2=7.8 Hz), 4.42 (d, 1H, J=2.4 Hz), 4.03 (d, 1H, J=1.8 Hz), 3.84 (s, 3H), 3.52 (s, 2H), 3.21-3.30 (m, 1H), 3.03-3.04 (m, 2H), 2.79-2.87 (m, 1H), 2.15 (s, 6H), 1.92-1.97 (m, 2H), 1.70-1.77 (m, 2H).

### Example 36. 3′-[(3,4-dichlorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 590)

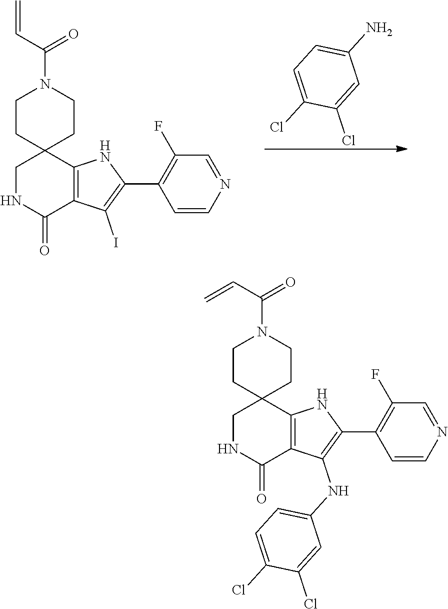 [SMILES: C=CC(=O)N1CCC2(CC1)CNC(=O)c1c2[nH]c(-c2ccncc2F)c1I ; Nc1ccc(Cl)c(Cl)c1 ; C=CC(=O)N1CCC2(CC1)CNC(=O)c1c2[nH]c(-c2ccncc2F)c1Nc1ccc(Cl)c(Cl)c1]

To a stirred solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (300 mg, 0.625 mmol, 1 equiv) and 3,4-dichloroaniline (121.44 mg, 0.750 mmol, 1.2 equiv) in DMF (4 mL) were added EPhos Pd G4 (114.75 mg, 0.125 mmol, 0.2 equiv), EPhos (66.81 mg, 0.125 mmol, 0.2 equiv) and Cs₂CO₃ (407.04 mg, 1.250 mmol, 2 equiv) under N2 atmosphere. The final reaction mixture was stirred for 2 h at 50 degrees. The resulting mixture was extracted with EA (3×10 mL). The combined organic layers were washed with brine (3×50 mL), dried over anhydrous sodium sulphate. After filtration, the filtrate was concentrated under reduced pressure. The crude product (90 mg) was purified by Prep-HPLC with the following conditions (Column: YMC-Actus Triart C18 ExRS, 30*150 mm, 5 μm; Mobile Phase A: Water (10 mmol/L NH₄HCO₃+0.1% NH₃*H₂O), Mobile Phase B: ACN; Flow rate: 60 mL/min; Gradient: 28% B to 50% B in 8 min, 50% B; Wave Length: 254/220 nm; RT1 (min): 7.43) to afford 3′-[(3,4-dichlorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (12.5 mg, 3.86%) as a white solid.

LC-MS: (M+H)⁺ found: 514.1.

¹H NMR (400 MHz, DMSO-d6) δ 11.47 (s, 1H), 8.52 (d, J=2.4 Hz, 1H), 8.34 (dd, J=5.1, 1.1 Hz, 1H), 7.80 (s, 1H), 7.42 (dd, J=6.6, 5.1 Hz, 1H), 7.26-7.14 (m, 2H), 6.87 (dd, J=16.7, 10.5 Hz, 1H), 6.66 (d, J=2.7 Hz, 1H), 6.51 (dd, J=8.8, 2.7 Hz, 1H), 6.15 (dd, J=16.7, 2.5 Hz, 1H), 5.79-5.67 (m, 1H), 4.42 (d, J=13.4 Hz, 1H), 4.05 (d, J=14.0 Hz, J H), 3.51 (s, 2H), 3.32-3.25 (m, 1H), 2.84 (t, J=13.0 Hz, J H), 2.04-1.89 (m, 2H), 1.76 (t, J=12.3 Hz).

### Example 37. 3′-[(2,3-dichlorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 589)

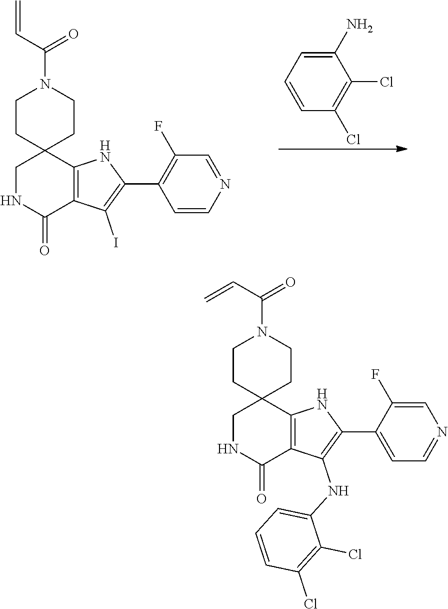 [SMILES: C=CC(=O)N1CCC2(CC1)CNC(=O)c1c2[nH]c(-c2ccncc2F)c1I ; Nc1cccc(Cl)c1Cl ; C=CC(=O)N1CCC2(CC1)CNC(=O)c1c2[nH]c(-c2ccncc2F)c1Nc1cccc(Cl)c1Cl]

To a stirred solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (300 mg, 0.625 mmol, 1.0 equiv) and 2,3-dichloroaniline (202.39 mg, 1.250 mmol, 2.0 equiv) in DMF (10 mL) were added Cs₂CO₃ (407.04 mg, 1.250 mmol, 2.0 equiv), EPhos (66.81 mg, 0.125 mmol, 0.2 equiv) and EPhos Pd G4 (114.75 mg, 0.125 mmol, 0.2 equiv) in portions at room temperature under nitrogen atmosphere. The resulting mixture was stirred for 3 h at 50° C. under nitrogen atmosphere. The resulting mixture was diluted with water. The resulting mixture was extracted with CH₂Cl₂ (3×100 mL). The combined organic layers were dried over anhydrous Na₂SO₄. After filtration, the filtrate was concentrated under reduced pressure. The crude product (200 mg) was purified by Prep-HPLC with the following conditions (Column: YMC-Actus Triart C18, 30*150 mm, 5 μm; Mobile Phase A: Water (10 mmol/L NH₄HCO₃+0.1% NH₃*H₂O), Mobile Phase B: ACN; Flow rate: 60 m/min; Gradient: 37% B to 48% B in 8 min, 48% B; Wave Length: 254; 220 nm; RT1 (min): 8.05) to afford 3′-[(2,3-dichlorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (46.4 mg, 14.39%) as a off-white solid.

LC-MS: M+H found: 514.

¹H NMR (300 MHz, DMSO-d6) δ 11.51 (s, 1H), 8.48 (d, J=2.6 Hz, 1H), 8.36-8.28 (m, 1H), 7.75 (s, 1H), 7.47-7.32 (m, 2H), 6.94-6.80 (m, 3H), 6.30-6.21 (m, 1H), 6.15 (dd, J=16.7, 2.5 Hz, 1H), 5.71 (dd, J=10.3, 2.5 Hz, 1H), 4.42 (d, J=12.6 Hz, 1H), 4.05 (d, J=13.1 Hz, 1H), 3.53 (s, 2H), 2.88-2.78 (m, 1H), 1.96 (s, 2H), 1.77 (s, 2H).

### Example 38. 3′-[(4-chloro-3-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 588)

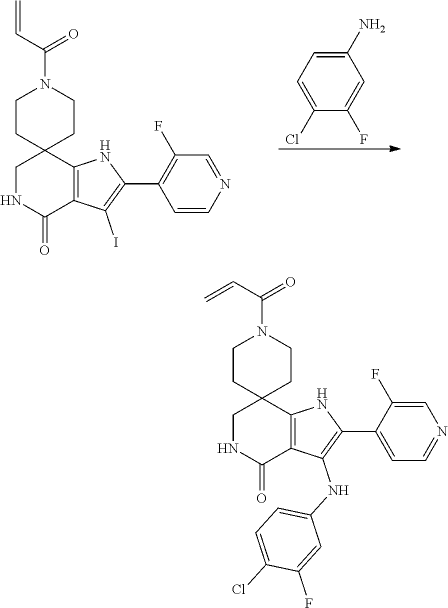 [SMILES: C=CC(=O)N1CCC2(CC1)CNC(=O)c1c2[nH]c(-c2ccncc2F)c1I ; Nc1ccc(Cl)c(F)c1 ; C=CC(=O)N1CCC2(CC1)CNC(=O)c1c2[nH]c(-c2ccncc2F)c1Nc1ccc(Cl)c(F)c1]

To a stirred solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (300 mg, 0.625 mmol, 1.0 equiv) and 4-chloro-3-fluoroaniline (181.84 mg, 1.250 mmol, 2.0 equiv) in DMF (10 mL) were added Cs₂CO₃ (407.04 mg, 1.250 mmol, 2.0 equiv), EPhos (66.81 mg, 0.125 mmol, 0.2 equiv) and EPhos Pd G4 (114.75 mg, 0.125 mmol, 0.2 equiv) in portions at room temperature under nitrogen atmosphere. The resulting mixture was stirred for 3 h at 50° C. under nitrogen atmosphere. The resulting mixture was diluted with water. The resulting mixture was extracted with CH₂Cl₂ (3×100 mL). The combined organic layers were dried over anhydrous Na₂SO₄. After filtration, the filtrate was concentrated under reduced pressure. The crude product (200 mg) was purified by Prep-HPLC with the following conditions (Column: YMC-Actus Triart C18, 30*150 mm, 5 μm; Mobile Phase A: Water (10 mmol/L NH₄HCO₃+0.1% NH₃*H₂O), Mobile Phase B: ACN; Flow rate: 60 mL/min; Gradient: 35% B to 47% B in 8 min, 47% B; Wave Length: 254; 220 nm; RT1 (min): 7.7) to afford 3′-[(4-chloro-3-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (51.1 mg, 16.41%) as a white solid.

LC-MS: M+H found: 498.

¹H NMR (400 MHz, DMSO-d6) δ 11.49 (s, 1H), 8.51 (d, J=2.5 Hz, 1H), 8.37-8.31 (m, 1H), 7.62 (d, J=2.0 Hz, 1H), 7.46 (dd, J=6.7, 5.0 Hz, 1H), 7.33 (s, 1H), 6.88 (dd, J=16.6, 10.5 Hz, 1H), 6.78-6.68 (m, 2H), 6.30-6.21 (m, 1H), 6.15 (dd, J=16.6, 2.5 Hz, 1H), 5.72 (dd, J=10.5, 2.5 Hz, 1H), 4.43 (d, J=13.2 Hz, 1H), 4.06 (d, J=14.1 Hz, 1H), 3.52 (s, 2H), 3.27 (d, J=13.3 Hz, 1H), 2.85 (t, J=12.9 Hz, 1H), 1.98 (d, J=13.5 Hz, 2H), 1.76 (t, J=11.9 Hz, 2H).

### Example 39. 3′-[(3-chloro-4-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 587)

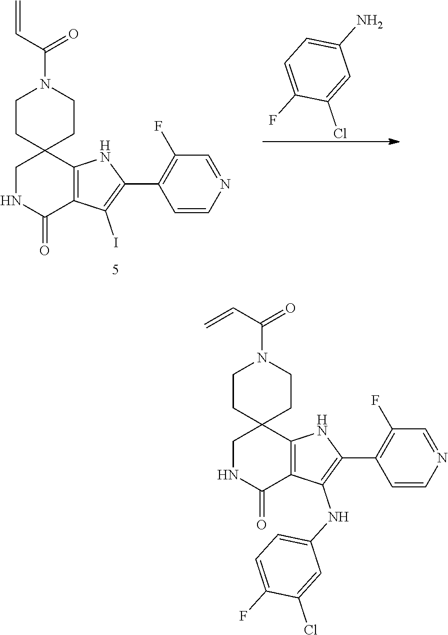 [SMILES: C=CC(=O)N1CCC2(CC1)CNC(=O)c1c2[nH]c(-c2ccncc2F)c1I ; Nc1ccc(F)c(Cl)c1 ; C=CC(=O)N1CCC2(CC1)CNC(=O)c1c2[nH]c(-c2ccncc2F)c1Nc1ccc(F)c(Cl)c1]

To a stirred solution of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (300 mg, 0.625 mmol, 1 equiv) and 3-chloro-4-fluoroaniline (109.11 mg, 0.750 mmol, 1.2 equiv) in DMF (5 mL) were added EPhos Pd G4 (114.75 mg, 0.125 mmol, 0.2 equiv), EPhos (66.81 mg, 0.125 mmol, 0.2 equiv) and Cs₂CO₃ (407.04 mg, 1.250 mmol, 2 equiv) under N2 atmosphere. The final reaction mixture was irradiated with microwave radiation for 2 h at 50 degrees. The resulting mixture was filtered, the filter cake was washed with EA (3×5 mL). The filtrate was concentrated under reduced pressure. The resulting mixture was extracted with EA (3×20 mL). The combined organic layers were washed with brine (3×50 mL), dried over anhydrous Na₂SO₄. After filtration, the filtrate was concentrated under reduced pressure. The crude product (60 mg) was purified by Prep-HPLC with the following conditions (Column: YMC-Actus Triart C18, 30*150 mm, 5 μm; Mobile Phase A: Water (10 mmol/L NH₄HCO₃+0.1% NH₃*H₂O), Mobile Phase B: ACN; Flow rate: 60 mL/min; Gradient: 35% B to 45% B in 8 min, 45% B; Wave Length: 254: 220 nm; RT1 (min): 7.45) to afford 3′-[(3-chloro-4-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (28.0 mg, 8.98%) as a white solid.

LC-MS: (M+H)⁺ found: 498.1.

¹H NMR (400 MHz, DMSO-d6) δ 11.41 (s, 1H), 8.51 (d, J=2.5 Hz, 1H), 8.32 (dd, J=5.0, 1.1 Hz, 1H), 7.63 (s, 1H), 7.41 (dd, J=6.7, 5.0 Hz, 1H), 7.27 (d, J=2.9 Hz, 1H), 7.01 (t, J=9.1 Hz, 1H), 6.88 (dd, J=16.7, 10.5 Hz, 1H), 6.60 (dd, J=6.4, 2.8 Hz, 1H), 6.52-6.47 (m, 1H), 6.15 (dd, J=16.7, 2.5 Hz, 1H), 5.71 (dd, J=10.4, 2.5 Hz, 1H), 4.42 (d, J=13.4 Hz, 1H), 4.06 (d, J=14.0 Hz, 1H), 3.51 (t, J=2.9 Hz, 2H), 3.24 (s, 1H), 2.85 (t, J=13.0 Hz, 1H), 1.97 (q, J=11.8 Hz, 2H), 1.76 (t, J=12.1 Hz, 2H).

### Example 40. 3′-[(3-chloro-2-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 586)

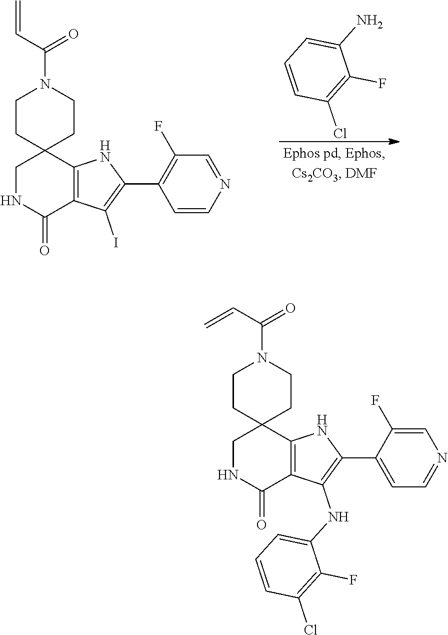 [SMILES: C=CC(=O)N1CCC2(CC1)CNC(=O)c1c2[nH]c(-c2ccncc2F)c1I ; Nc1cccc(Cl)c1F ; C=CC(=O)N1CCC2(CC1)CNC(=O)c1c2[nH]c(-c2ccncc2F)c1Nc1cccc(Cl)c1F]

To a stirred mixture of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (300 mg, 0.625 mmol, 1 equiv) and 3-chloro-2-fluoroaniline (181.84 mg, 1.250 mmol, 2 equiv) in DMF (5 mL) were added EPhos Pd G4 (86.06 mg, 0.094 mmol, 0.15 equiv), EPhos (100.22 mg, 0.188 mmol, 0.3 equiv) and Cs₂CO₃ (407.04 mg, 1.250 mmol, 2 equiv) in portions at room temperature under nitrogen atmosphere. The resulting mixture was stirred for 2 h at 50° C. under nitrogen atmosphere. The resulting mixture was extracted with EtOAc (2×100 mL). The combined organic layers were dried over anhydrous Na₂SO₄. After filtration, the filtrate was concentrated under reduced pressure. The residue was purified by reverse flash chromatography with the following conditions: column, silica gel; mobile phase, MeCN in water, 10% to 50% gradient in 10 min; detector, UV 254 nm. To afford 3′-[(3-chloro-2-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (100 mg), then the crude product (60 mg) was purified by Prep-HPLC with the following conditions (Column: XBridge Prep OBD C18 Column, 30*150 mm, 5 μm; Mobile Phase A: Water (10 mmol/L NH₄HCO₃+0. 1% NH₃*H₂O), Mobile Phase B: ACN; Flow rate: 60 mL/min; Gradient: 30% B to 45% B in 8 min, 45% B; Wave Length: 254; 220 nm; RT1 (min): 6.57) to afford 3′-[(3-chloro-2-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (19.1 mg, 6.09%) as an off-white solid.

¹H NMR (400 MHz, DMSO-d6) δ 1.75 (t, 2H), 1.91-2.03 (q, 2H), 2.85 (t, 1H), 3.28 (t, 1H), 3.49-3.55 (m, 2H), 4.05 (d, 1H), 4.42 (d, 1H), 5.71 (dd, 1H), 6.15 (dd, 1H), 6.19-6.30 (m, 1H), 6.67-6.77 (m, 2H), 6.87 (dd, 1H), 7.33 (d, 1H), 7.46 (dd, 1H), 7.62 (d, 1H), 8.33 (dd, 1H), 8.50 (d, 1H), 11.49 (s, 1H).

### Example 41. 3′-[(2,3-difluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (compound 585)

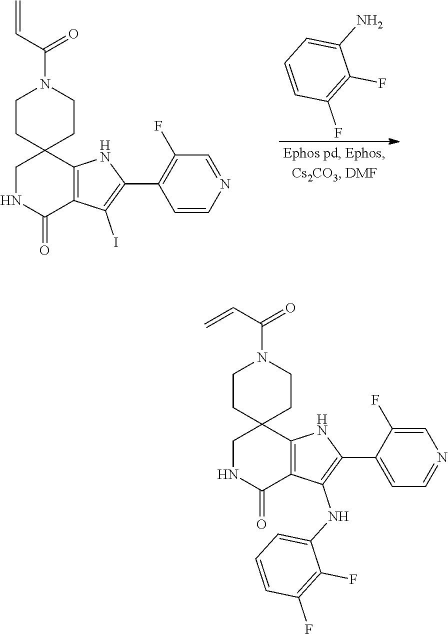 [SMILES: C=CC(=O)N1CCC2(CC1)CNC(=O)c1c2[nH]c(-c2ccncc2F)c1I ; Nc1cccc(F)c1F ; C=CC(=O)N1CCC2(CC1)CNC(=O)c1c2[nH]c(-c2ccncc2F)c1Nc1cccc(F)c1F]

To a stirred mixture of 2′-(3-fluoropyridin-4-yl)-3′-iodo-1-(prop-2-enoyl)-5′,6′-dihydro-1′H-spiro[piperidine-4,7′-pyrrolo[3,2-c]pyridin]-4′-one (300 mg, 0.625 mmol, 1 equiv) and 2,3-difluoroaniline (161.29 mg, 1.250 mmol, 2 equiv) in DMF (5 mL) were added EPhos Pd G4 (86.06 mg, 0.094 mmol, 0.15 equiv), EPhos (100.22 mg, 0.188 mmol, 0.3 equiv) and Cs₂CO₃ (407.04 mg, 1.250 mmol, 2 equiv) in portions at room temperature under nitrogen atmosphere. The resulting mixture was stirred for 2 h at 50° C. under nitrogen atmosphere. The resulting mixture was extracted with EtOAc (2×100 mL). The combined organic layers were dried over anhydrous Na₂SO₄. After filtration, the filtrate was concentrated under reduced pressure. The residue was purified by reverse flash chromatography with the following conditions: column, silica gel; mobile phase, MeCN in water, 10% to 50% gradient in 10 min; detector, UV 254 nm to afford 3′-[(3-chloro-2-fluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (100 mg). The semi-pure product (100 mg) was purified by Prep-HPLC with the following conditions (Column: XBridge Prep OBD C18 Column, 30*150 mm, 5 μm; Mobile Phase A: Water (10 mmol/L NH₄HCO₃+0.1% NH₃*H₂O), Mobile Phase B: ACN; Flow rate: 60 mL/min; Gradient: 30% B to 45% B in 8 min, 45% B; Wave Length: 254; 220 nm; RT1 (min): 6.57) to afford 3′-[(2,3-difluorophenyl)amino]-2′-(3-fluoropyridin-4-yl)-1-(prop-2-enoyl)-5′,6′-dihydro-1′H-spiro[piperidine-4,7′-pyrrolo[3,2-c]pyridin]-4′-one (41.6 mg, 13.75%) as a off-white solid.

¹H NMR (400 MHz, DMSO-d6) δ 1.75 (t, 2H), 1.90-2.03 (m, 2H), 2.85 (t, 1H), 3.28 (t, 1H), 3.52 (t, 2H), 4.05 (d, 1H), 4.42 (d, 1H), 5.71 (dd, 1H), 6.07-6.15 (m, 1H), 6.11-6.19 (m, 1H), 6.52-6.63 (m, 1H), 6.64-6.74 (m, 1H), 6.87 (dd, 1H), 7.33 (t, 1H), 7.46 (dd, 1H), 7.62 (d, 1H), 8.33 (dd, 1H), 8.49 (d, 1H), 11.48 (s, 1H).

### Example 42. 3-[(3-fluoro-2-methoxyphenyl)amino]-2-{2-methoxypyrido[3,2-d]pyrimidin-8-yl}-6-(trifluoromethyl)-1H,5H,6H,7H-pyrrolo[3,2-c]pyridin-4-one (compound 595)

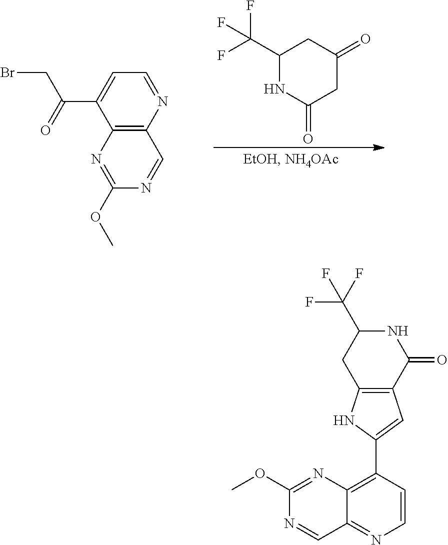 [SMILES: COc1ncc2nccc(C(=O)CBr)c2n1 ; O=C1CC(=O)NC(C(F)(F)F)C1 ; COc1ncc2nccc(-c3cc4c([nH]3)CC(C(F)(F)F)NC4=O)c2n1]

To a stirred solution of 2-bromo-1-{2-methoxypyrido[3,2-d]pyrimidin-8-yl}ethanone (1 g, 3.545 mmol, 1.0 equiv) and 6-(trifluoromethyl)piperidine-2,4-dione (0.71 g, 3.900 mmol, 1.1 equiv) in EtOH (15 mL) was added NH₄OAc (1.37 g, 17.725 mmol, 5.0 equiv) in portions at room temperature under nitrogen atmosphere. The resulting mixture was stirred for 6 h at 50° C. under nitrogen atmosphere. The aqueous layer was extracted with EtOAc (3×30 mL). The residue was purified by silica gel column chromatography, eluted with PE/EA (1:1) to afford 2-{2-methoxypyrido[3,2-d]pyrimidin-8-yl}-6-(trifluoromethyl)-1H,5H,6H,7H-pyrrolo[3,2-c]pyridin-4-one (850 mg, 66.00%) as a light yellow solid.

LC-MS: M+H found: 364.

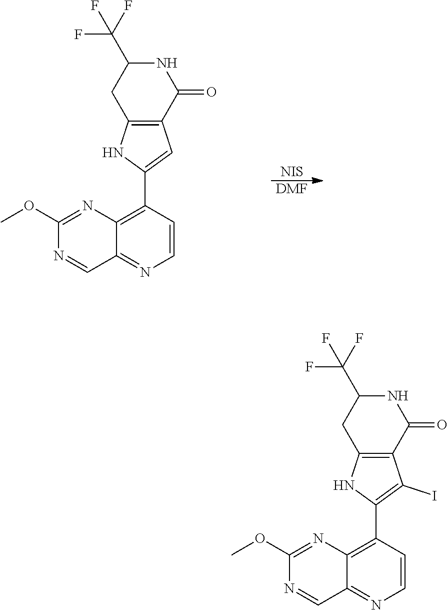

A solution of 2-{2-methoxypyrido[3,2-d]pyrimidin-8-yl}-6-(trifluoromethyl)-1H,5H,6H,7H-pyrrolo[3,2-c]pyridin-4-one (300 mg, 0.826 mmol, 1.00 equiv) and NIS (222.94 mg, 0.991 mmol, 1.2 equiv) in DMF (5 mL) was stirred for 1 h at 40 degrees C. The residue was purified by reverse flash chromatography with the following conditions: column, silica gel; mobile phase, MeCN in water; detector, UV 254 nm to afford 3-iodo-2-{2-methoxypyrido[3,2-d]pyrimidin-8-yl}-6-(trifluoromethyl)-1H,5H,6H,7H-pyrrolo[3,2-c]pyridin-4-one (90 mg, 22.28%) as a yellow solid.

LC-MS: (M+H)⁺ found: 490.

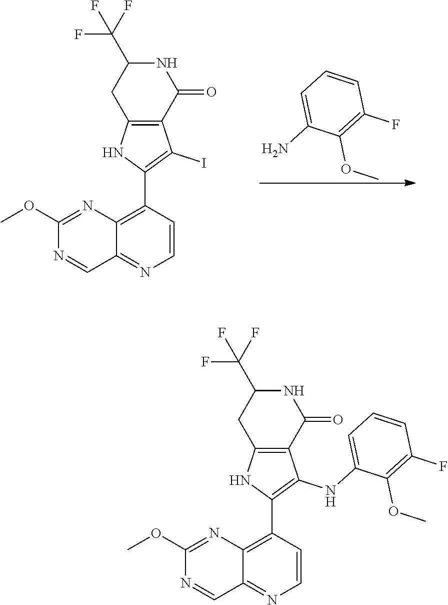

To a solution of 3-iodo-2-{2-methoxypyrido[3,2-d]pyrimidin-8-yl}-6-(trifluoromethyl)-1H,5H,6H,7H-pyrrolo[3,2-c]pyridin-4-one (70 mg, 0.143 mmol, 1 equiv) and 3-fluoro-2-methoxyaniline (40.39 mg, 0.286 mmol, 2 equiv) in DMF (2 mL) were added Cs₂CO₃ (139.87 mg, 0.429 mmol, 3 equiv) and EPhos Pd G4 (26.29 mg, 0.029 mmol, 0.2 equiv), EPhos (15.30 mg, 0.029 mmol, 0.2 equiv). After stirring for 3 h at 50° C. under a nitrogen atmosphere, the resulting mixture was concentrated under reduced pressure. The residue was purified by reverse flash chromatography with the following conditions: column, silica gel; mobile phase, MeCN in water; detector, UV 254 nm to afford 3-[(3-fluoro-2-methoxyphenyl)amino]-2-{2-methoxypyrido[3,2-d]pyrimidin-8-yl}-6-(trifluoromethyl)-1H,5H,6H,7H-pyrrolo[3,2-c]pyridin-4-one (5 mg, 6.27%) as an orange solid.

LC-MS: (M+H)⁺ found 503.

¹H NMR (400 MHz, Chloroform-d) δ 14.26 (s, 1H), 13.48 (d, J=4.8 Hz, 1H), 12.69 (d, J=4.3 Hz, 1H), 12.50 (s, 1H), 12.43 (s, 1H), 12.42 (s, 1H), 11.40-11.35 (m, 1H), 11.27 (dd, J=10.7, 1.7 Hz, 1H), 10.76 (d, J=8.2 Hz, 1H), 9.22 (s, 1H), 8.97 (s, 3H), 8.63 (d, J=0.9 Hz, 3H), 5.99 (s, 2H).

### Example 43. 3-[(3-chloro-2-methoxyphenyl)amino]-7-(2-fluoroethyl)-2-(3-fluoropyridin-4-yl)-1H,5H,6H,7H-pyrrolo[3,2-c]pyridin-4-one (compound 473)

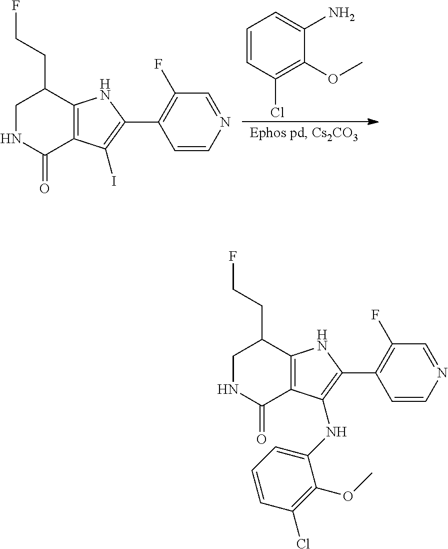

To a stirred mixture of 7-(2-fluoroethyl)-2-(3-fluoropyridin-4-yl)-3-iodo-1H,5H,6H,7H-pyrrolo[3,2-c]pyridin-4-one (400 mg, 0.992 mmol, 1 equiv), Cs₂CO₃ (646.51 mg, 1.984 mmol, 2 equiv) and 3-chloro-2-methoxyaniline (234.54 mg, 1.488 mmol, 1.5 equiv) in DMF (5 mL) were added EPhos Pd G4 (91.13 mg, 0.099 mmol, 0.1 equiv) and EPhos (106.12 mg, 0.198 mmol, 0.2 equiv) in portions at room temperature under nitrogen atmosphere. The resulting mixture was stirred for 2 h at 50° C. under nitrogen atmosphere. The resulting mixture was extracted with EtOAc (2×100 mL). The combined organic layers were dried over anhydrous Na₂SO₄. After filtration, the filtrate was concentrated under reduced pressure. The residue was purified by reverse flash chromatography with the following conditions: column, silica gel; mobile phase, MeCN in water, 10% to 100% gradient in 20 min; detector, UV 254 nm. to afford 3-[(3-chloro-2-methoxyphenyl)amino]-7-(2-fluoroethyl)-2-(3-fluoropyridin-4-yl)-1H,5H,6H,7H-pyrrolo[3,2-c]pyridin-4-one (12.6 mg, 39.04%) as an off-white solid.

¹H NMR (400 MHz, DMSO-d6) δ 1.89-2.05 (m, 1H), 2.09-2.26 (m, 1H), 3.15 (dq, 1H), 3.23-3.32 (m, 1H), 3.54-3.61 (m, 1H), 3.86 (s, 3H), 4.55 (t, 1H), 4.67 (t, 1H), 6.13 (dd, 1H), 6.61-6.70 (m, 2H), 7.21 (t, 1H), 7.46 (dd, 1H), 7.60 (s, 1H), 8.31 (dd, 11H), 8.51 (d, 1H), 11.61 (s, 1H).

### Example 44. 2′-(2-aminopyrimidin-4-yl)-3′-[(3-chloro-2-methoxyphenyl)amino]-1-(prop-2-enoyl)-5′,6′-dihydro-1′H-spiro[piperidine-4,7′-pyrrolo[3,2-c]pyridin]-4′-one (compound 556)

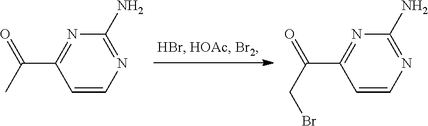

To a stirred mixture of 1-(2-aminopyrimidin-4-yl) ethanone (8.3 g, 60.521 mmol, 1 equiv) and HBr in water (2.45 g, 30.261 mmol, 0.5 equiv) in HOAc (50 mL) was added Br₂ (9.67 g, 60.521 mmol, 1 equiv) dropwise at room temperature under nitrogen atmosphere. The resulting mixture was stirred for overnight at room temperature under nitrogen atmosphere. The resulting mixture was diluted with EtOAc. The precipitated solids were collected by filtration and washed with EtOAc. This resulted in 1-(2-aminopyrimidin-4-yl)-2-bromoethanone (12.3 g, 94.07%) as a yellow solid.

LC-MS: M+H found: 216.

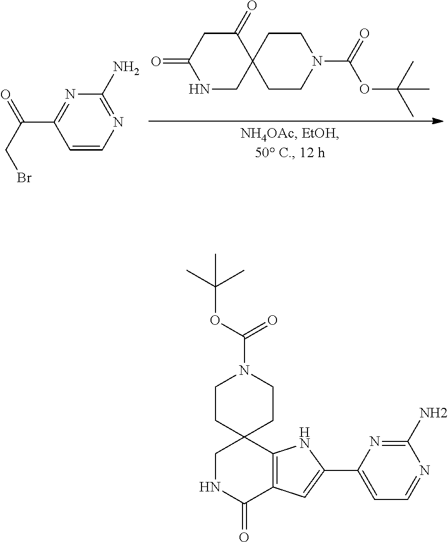

A mixture of 1-(2-aminopyrimidin-4-yl)-2-bromoethanone (1 g, 4.629 mmol, 1 equiv) and NH₄OAc (1.43 g, 18.516 mmol, 4 equiv) in EtOH (20 mL) was stirred for overnight at 60° C. under nitrogen atmosphere. The resulting mixture was concentrated under reduced pressure. The resulting mixture was diluted with water. The precipitated solids were collected by filtration and washed with MeOH. This resulted in tert-butyl 2′-(2-aminopyrimidin-4-yl)-4′-oxo-5′,6′-dihydro-1′H-spiro[piperidine-4,7′-pyrrolo[3,2-c]pyridine]-1-carboxylate (1.2 g, 65.06%) as a yellow solid.

LC-MS: (M+H)⁺ found: 399.

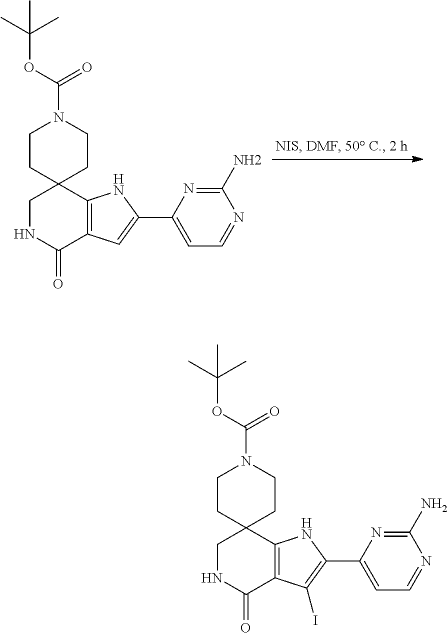 [SMILES: CC(C)(C)OC(=O)N1CCC2(CC1)CNC(=O)c1cc(-c3ccnc(N)n3)[nH]c12 ; CC(C)(C)OC(=O)N1CCC2(CC1)CNC(=O)c1c2[nH]c(-c2ccnc(N)n2)c1I]

A mixture of tert-butyl 2′-(2-aminopyrimidin-4-yl)-4′-oxo-5′,6′-dihydro-1′H-spiro[piperidine-4,7′-pyrrolo[3,2-c] pyridine]-1-carboxylate (850 mg, 2.133 mmol, 1 equiv) and NIS (527.92 mg, 2.346 mmol, 1.1 equiv) in DMF (20 mL) was stirred for 2 h at 50° C. under nitrogen atmosphere. The resulting mixture was diluted with water. The precipitated solids were collected by filtration and washed with water. This resulted in tert-butyl 2′-(2-aminopyrimidin-4-yl)-3′-iodo-4′-oxo-5′,6′-dihydro-1′H-spiro[piperidine-4,7′-pyrrolo[3,2-c] pyridine]-1-carboxylate (1.1 g, 98.34%) as a white solid.

LC-MS: (M+H)⁺ found: 525.

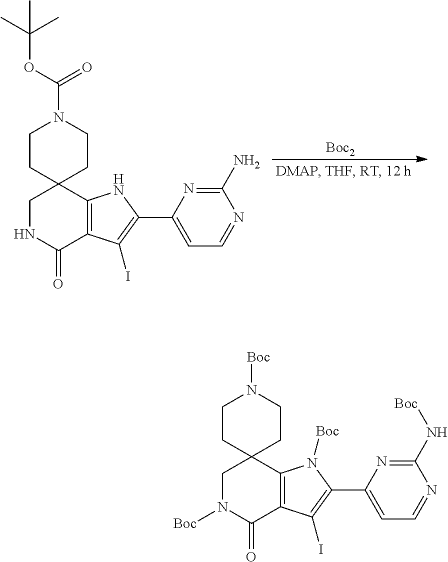 [SMILES: CC(C)(C)OC(=O)N1CCC2(CC1)CNC(=O)c1c2[nH]c(-c2ccnc(N)n2)c1I ; "Boc2" ; CC(C)(C)OC(=O)Nc1nccc(-c2c(I)c3c(n2C(=O)OC(C)(C)C)C2(CCN(C(=O)OC(C)(C)C)CC2)CN(C(=O)OC(C)(C)C)C3=O)n1]

A mixture of tert-butyl 2′-(2-aminopyrimidin-4-yl)-3′-iodo-4′-oxo-5′,6′-dihydro-1′H-spiro[piperidine-4,7′-pyrrolo[3,2-c]pyridine]-1-carboxylate (2 g, 3.814 mmol, 1 equiv) and DMAP (46.60 mg, 0.381 mmol, 0.1 equiv) in THF (20 mL) was stirred for overnight at room temperature under air atmosphere. The resulting mixture was diluted with water. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (5:1) to afford 1,1′,5′-tri-tert-butyl 2′-{2-[(tert-butoxycarbonyl)amino]pyrimidin-4-yl}-3′-iodo-4′-oxo-6′H-spiro[piperidine-4,7′-pyrrolo[3,2-c]pyridine]-1,1′,5′-tricarboxylate (2 g, 63.58%) as a white solid.

LC-MS: (M+H)⁺ found: 825.

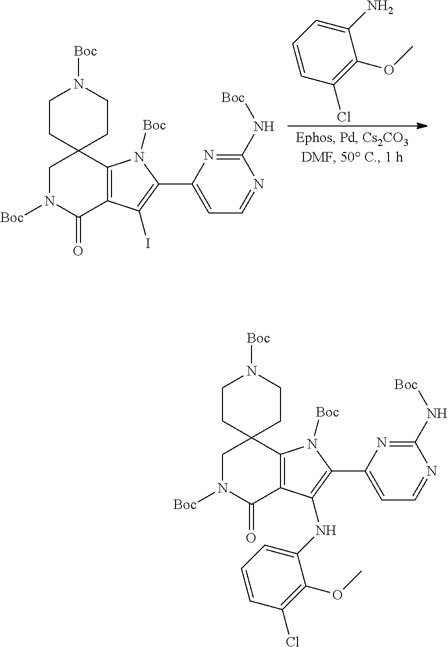

A mixture of 1,1′,5′-tri-tert-butyl 2′-{2-[(tert-butoxycarbonyl)amino]pyrimidin-4-yl}-3′-iodo-4′-oxo-6′H-spiro[piperidine-4,7′-pyrrolo[3,2-c]pyridine]-1,1′,5′-tricarboxylate (380 mg, 0.461 mmol, 1 equiv), 3-chloro-2-methoxyaniline (217.85 mg, 1.383 mmol, 3 equiv), EPhos Pd G4 (42.32 mg, 0.046 mmol, 0.1 equiv), EPhos (49.28 mg, 0.092 mmol, 0.2 equiv) and Cs₂CO₃ (450.38 mg, 1.383 mmol, 3 equiv) in DMF (9 mL) was stirred for 2 h at 50° C. under nitrogen atmosphere. The resulting mixture was diluted with water. The resulting mixture was extracted with EtOAc. The combined organic layers were washed with brine, dried over anhydrous Na2SO4. After filtration, the filtrate was concentrated under reduced pressure. The residue was purified by silica gel column chromatography, eluted with PE/EA (5:1) to afford 1,1′,5′-tri-tert-butyl 2′-{2-[(tert-butoxycarbonyl)amino]pyrimidin-4-yl}-3′-[(3-chloro-2-methoxyphenyl)amino]-4′-oxo-6′H-spiro[piperidine-4,7′-pyrrolo[3,2-c]pyridine]-1,1′,5′-tricarboxylate (160 mg, 40.64%) as a white solid.

LC-MS: (M+H)⁺ found: 854.

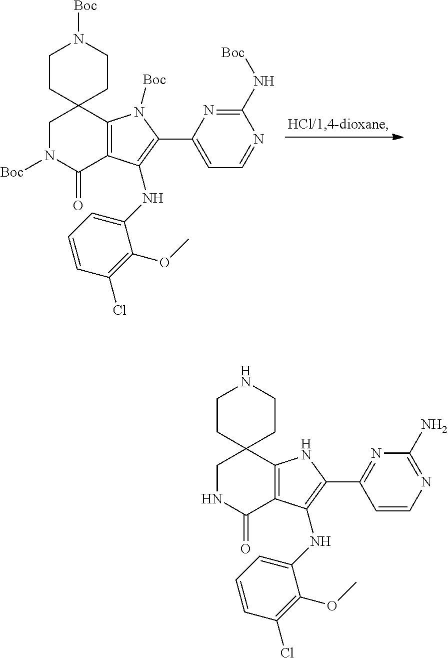 [SMILES: COc1c(Cl)cccc1Nc1c2c(n(C(=O)OC(C)(C)C)c1-c1ccnc(NC(=O)OC(C)(C)C)n1)C1(CCN(C(=O)OC(C)(C)C)CC1)CN(C(=O)OC(C)(C)C)C2=O ; COc1c(Cl)cccc1Nc1c(-c2ccnc(N)n2)[nH]c2c1C(=O)NCC21CCNCC1]

A mixture of 1,1′,5′-tri-tert-butyl 2′-{2-[(tert-butoxycarbonyl)amino]pyrimidin-4-yl}-3′-[(3-chloro-2-methoxyphenyl)amino]-4′-oxo-6′H-spiro[piperidine-4,7′-pyrrolo[3,2-c]pyridine]-1,1′,5′-tricarboxylate (160 mg, 0.187 mmol, 1 equiv) in HCl (gas) in 1,4-dioxane (4 mL) was stirred for 2 h at room temperature under air atmosphere. The resulting mixture was concentrated under reduced pressure. The mixture/residue was acidified/basified/neutralized to pH 8 with saturated NaHCO₃ (aq.). The resulting mixture was extracted with CH₂Cl₂. The combined organic layers were washed with brine, dried over anhydrous Na₂SO₄. After filtration, the filtrate was concentrated under reduced pressure. The crude product was used in the next step directly without further purification. This resulted in 2′-(2-aminopyrimidin-4-yl)-3′-[(3-chloro-2-methoxyphenyl)amino]-5′,6′-dihydro-1′H-spiro[piperidine-4,7′-pyrrolo[3,2-c]pyridin]-4′-one (70 mg, 82.35%) as a brown solid.

LC-MS: (M+H)⁺ found: 454.

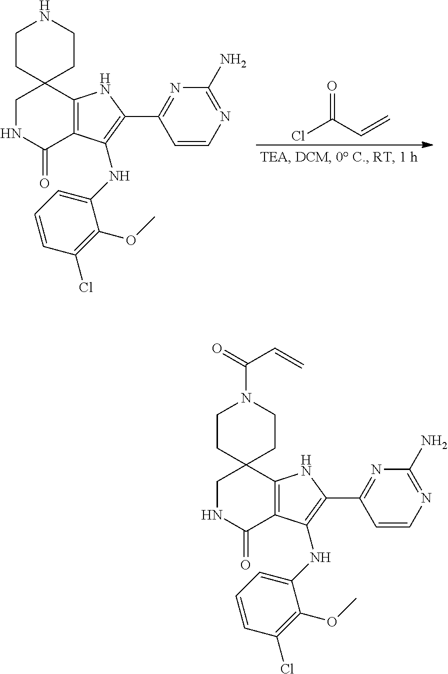

To a stirred mixture of 2′-(2-aminopyrimidin-4-yl)-3′-[(3-chloro-2-methoxyphenyl)amino]-5′,6′-dihydro-1′H-spiro[piperidine-4,7′-pyrrolo[3,2-c]pyridin]-4′-one (60 mg, 0.132 mmol, 1 equiv) and TEA (40.13 mg, 0.396 mmol, 3 equiv) in DCM (2 mL) was added acryloyl chloride (14.36 mg, 0.158 mmol, 1.2 equiv) dropwises at 0° C. under nitrogen atmosphere. The resulting mixture was stirred for 1 h at room temperature under nitrogen atmosphere. The reaction was quenched by the addition of Water/ice at 0° C. The resulting mixture was extracted with CH₂Cl₂. The combined organic layers were dried over anhydrous Na₂SO₄. After filtration, the filtrate was concentrated under reduced pressure. The crude product was purified by Prep-HPLC with the following conditions (Column: YMC-Actus Triart C18, 30*150 mm, 5 μm; Mobile Phase A: Water (10 mmol/L NH₄HCO₃+0.1% NH₃*H₂O), Mobile Phase B: ACN; Flow rate: 60 mL/min; Gradient: 32% B to 42% B in 8 min, 42% B; Wave Length: 254; 220 nm; RT1 (min): 7.58) to afford 2′-(2-aminopyrimidin-4-yl)-3′-[(3-chloro-2-methoxyphenyl)amino]-1-(prop-2-enoyl)-5′,6′-dihydro-1′H-spiro[piperidine-4,7′-pyrrolo[3,2-c]pyridin]-4′-one (9.3 mg, 13.85%) as a light yellow solid.

LC-MS: (M+H)⁺ found: 508.

¹H NMR (300 MHz, DMSO-d6) δ 11.40 (s, 1H), 8.31 (s, 1H), 8.08 (d, J=5.4 Hz, 1H), 7.29 (s, 1H), 6.94-6.68 (m, 3H), 6.56 (d, J=5.3 Hz, 1H), 6.38 (dd, J=7.8, 1.9 Hz, 1H), 6.24 (s, 2H), 6.16 (dd, J=16.7, 2.5 Hz, 1H), 5.79-5.66 (m, 1H), 4.45 (d, J=13.3 Hz, 1H), 4.05 (d, J=13.8 Hz, 1H), 3.89 (s, 3H), 3.50 (d, J=3.0 Hz, 2H), 3.28-3.13 (m, 1H), 2.81 (t, J=13.0 Hz, 1H), 2.12 (s, 2H), 1.69 (d, J=13.2 Hz, 2H).

### Example 45. 1-(but-2-ynoyl)-3′-[(3-fluoro-2-methoxyphenyl)amino]-2′-(3-fluoropyridin-4-yl)-5′,6′-dihydro-1′H-spiro[piperidine-4,7′-pyrrolo[3,2-c]pyridin]-4′-one (compound 609)

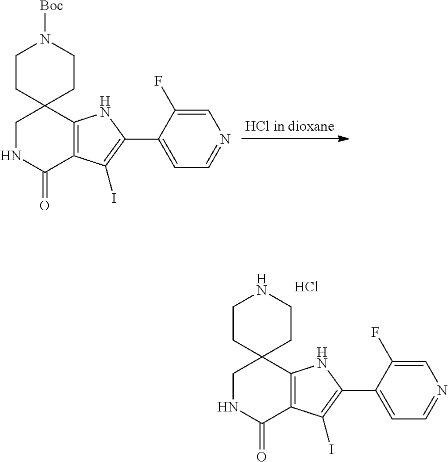 [SMILES: CC(C)(C)OC(=O)N1CCC2(CC1)CNC(=O)c1c2[nH]c(-c2ccncc2F)c1I ; Cl ; O=C1NCC2(CCNCC2)c2[nH]c(-c3ccncc3F)c(I)c21]

A mixture of tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (500 mg, 0.950 mmol, 1 equiv) in HCl (gas) in 1,4-dioxane (10 mL) was stirred for 2 h at room temperature under nitrogen atmosphere. The resulting mixture was concentrated under reduced pressure. This resulted in 2′-(3-fluoropyridin-4-yl)-3′-iodo-5′,6′-dihydro-1′H-spiro[piperidine-4,7′-pyrrolo[3,2-c]pyridin]-4′-one (350 mg, 86.44%) as a brown solid.

LC-MS: M+H found: 427.

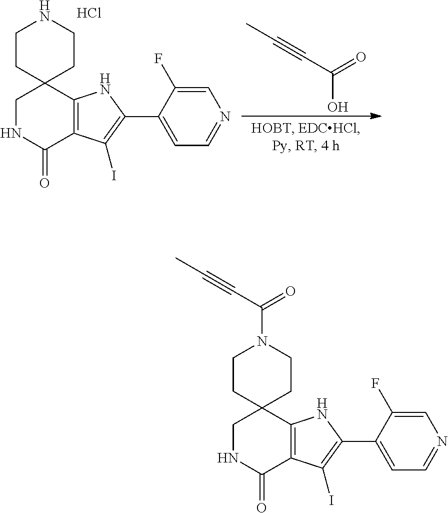

A mixture of 2′-(3-fluoropyridin-4-yl)-3′-iodo-5′,6′-dihydro-1′H-spiro[piperidine-4,7′-pyrrolo[3,2-c]pyridin]4′-one (300 mg, 0.704 mmol, 1 equiv) and EDC*HCl (202.39 mg, 1.056 mmol, 1.5 equiv) in Pyridine (6 mL) was stirred for overnight at room temperature under nitrogen atmosphere. The resulting mixture was diluted with water. The resulting mixture was extracted with EtOAc. The combined organic layers were washed with brine, dried over anhydrous Na₂SO₄. After filtration, the filtrate was concentrated under reduced pressure. The residue was purified by Prep-TLC (CH₂Cl₂/MeOH 15:1) to afford 1-(but-2-ynoyl)-2′-(3-fluoropyridin-4-yl)-3′-iodo-5′,6′-dihydro-1′H-spiro[piperidine-4,7′-pyrrolo[3,2-c]pyridin]-4′-one (300 mg, 86.58%) as a yellow solid.

LC-MS: (M+H)⁺ found: 493.

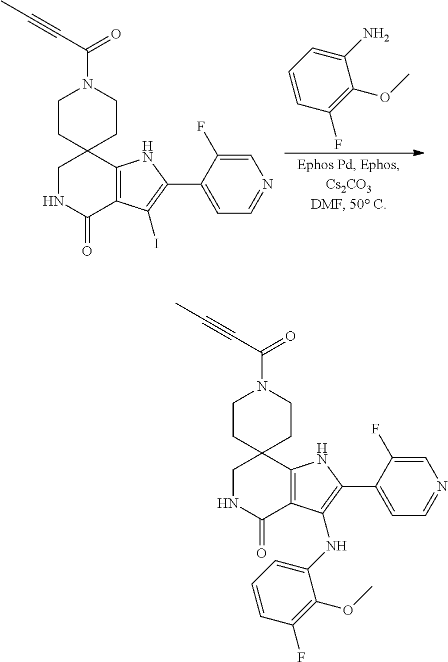

A mixture of 1-(but-2-ynoyl)-2′-(3-fluoropyridin-4-yl)-3′-iodo-5′,6′-dihydro-1′H-spiro[piperidine-4,7′-pyrrolo[3,2-c]pyridin]-4′-one (280 mg, 0.569 mmol, 1 equiv), 3-fluoro-2-methoxyaniline (240.84 mg, 1.707 mmol, 3 equiv), EPhos Pd G4 (52.24 mg, 0.057 mmol, 0.1 equiv), EPhos (60.83 mg, 0.114 mmol, 0.2 equiv) and Cs₂CO₃ (555.95 mg, 1.707 mmol, 3 equiv) in DMF (6 mL) was stirred for 2 h at 50° C. under nitrogen atmosphere. The resulting mixture was diluted with water. The resulting mixture was extracted with EtOAc. The combined organic layers were washed with brine, dried over anhydrous Na₂SO₄. After filtration, the filtrate was concentrated under reduced pressure. The crude product (mg) was purified by Prep-HPLC with the following conditions to afford 1-(but-2-ynoyl)-3′-[(3-fluoro-2-methoxyphenyl)amino]-2′-(3-fluoropyridin-4-yl)-5′,6′-dihydro-1′H-spiro[piperidine-4,7′-pyrrolo[3,2-c]pyridin]-4′-one (120 mg, 41.74%) as a white solid.

LC-MS: (M+H)⁺ found: 506.

¹H NMR (400 MHz, DMSO-d6) δ 11.39 (s, 11H), 8.48 (d, J=2.6 Hz, 1H), 8.32 (dd, J=5.1, 1.2 Hz, 1H), 7.68 (s, 1H), 7.45 (dd, J=6.7, 5.1 Hz, 1H), 7.36 (d, J=2.7 Hz, 1H), 6.57 (td, J=8.3, 6.1 Hz, 1H), 6.46 (ddd, J=10.8, 8.3, 1.5 Hz, 1H), 5.94 (dt, J=8.2, 1.4 Hz, 1H), 4.29 (d, J=13.3 Hz, 1H), 4.22 (d, J=13.5 Hz, 1H), 3.88 (s, 3H), 3.52 (t, J=2.6 Hz, 2H), 2.94-2.83 (m, 1H), 2.12-2.00 (m, 1H), 2.05 (s, 3H), 1.92 (td, J=13.2, 4.7 Hz, 1H), 1.80 (d, J=13.3 Hz, 1H), 1.74 (d, J=13.3 Hz, 1H).

### Example 46. 3′-[(3-fluoro-2-methoxyphenyl)amino]-1-(prop-2-enoyl)-2′-(pyrimidin-4-yl)-5′,6′-dihydro-1′H-spiro[piperidine-4,7′-pyrrolo[3,2-c]pyridin]-4′-one (compound 611)

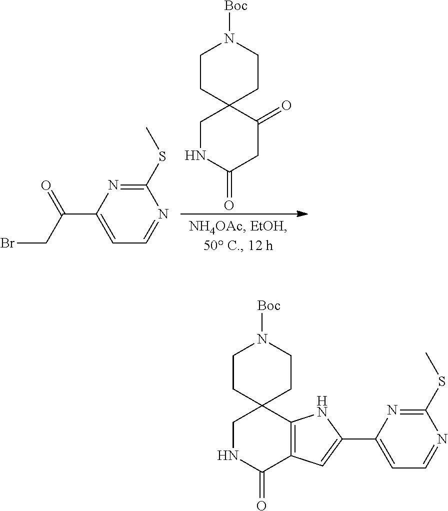

A mixture of 2-bromo-1-[2-(methylsulfanyl)pyrimidin-4-yl]ethanone (1.7 g, 6.880 mmol, 1 equiv), tert-butyl 3,5-dioxo-2,9-diazaspiro[5.5]undecane-9-carboxylate (2.14 g, 7.568 mmol, 1.1 equiv) and NH₄OAc (2.12 g, 27.520 mmol, 4 equiv) in EtOH (50 mL) was stirred for overnight at 50° C. under nitrogen atmosphere. The resulting mixture was concentrated under reduced pressure. The resulting mixture was diluted with water. The precipitated solids were collected by filtration and washed with acetonitrile. The crude product mixture was used in the next step directly without further purification.

LC-MS: M+H found: 430.

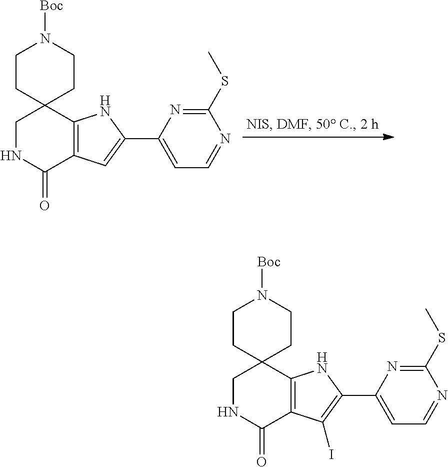 [SMILES: CSc1nccc(-c2cc3c([nH]2)C2(CCN(C(=O)OC(C)(C)C)CC2)CNC3=O)n1 ; CSc1nccc(-c2[nH]c3c(c2I)C(=O)NCC32CCN(C(=O)OC(C)(C)C)CC2)n1]

A mixture of tert-butyl 2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (6.3 g, 14.667 mmol, 1 equiv) and NIS (3.63 g, 16.134 mmol, 1.1 equiv) in DMF (100 mL) was stirred for 2 h at 50° C. under nitrogen atmosphere. The resulting mixture was diluted with water. The resulting mixture was extracted with CH₂Cl₂. The combined organic layers were washed with brine, dried over anhydrous Na₂SO₄. After filtration, the filtrate was concentrated under reduced pressure. The residue was purified by silica gel column chromatography, eluted with CH₂Cl₂/MeOH (50:1) to afford tert-butyl 3′-iodo-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (7.8 g, 95.75%) as a yellow solid.

LC-MS: (M+H)⁺ found: 556.

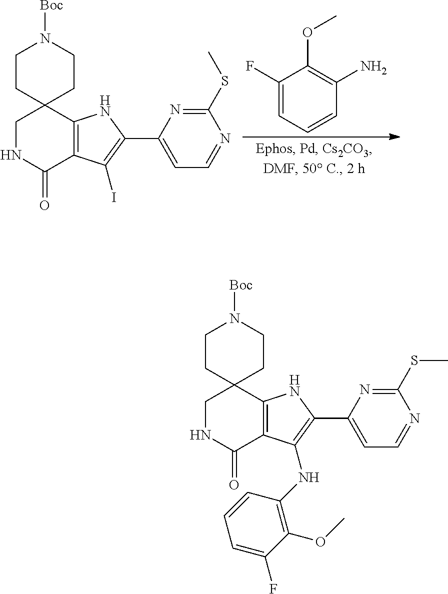

A mixture of tert-butyl 3′-iodo-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (1.5 g, 2.701 mmol, 1 equiv) and Cs₂CO₃ (2.64 g, 8.103 mmol, 3 equiv) in DMF (30 mL) was stirred for 2 h at 50° C. under nitrogen atmosphere. The resulting mixture was diluted with water. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with CH₂Cl₂/MeOH (50:1) to afford tert-butyl 3′-[(3-fluoro-2-methoxyphenyl)amino]-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (1.1 g, 71.63%) as a yellow solid.

LC-MS: (M+H)⁺ found: 569.

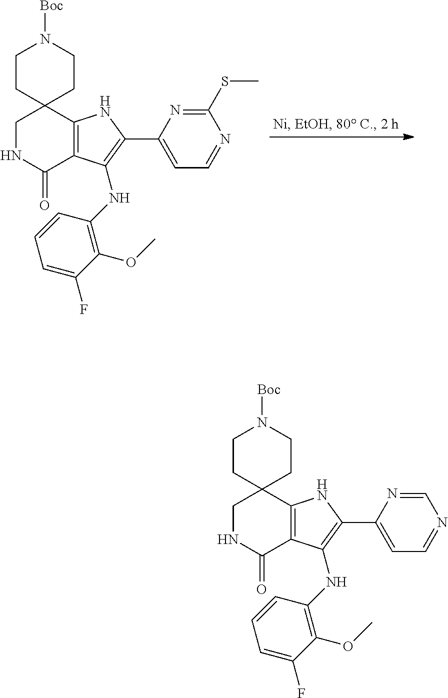 [SMILES: COc1c(F)cccc1Nc1c(-c2ccnc(SC)n2)[nH]c2c1C(=O)NCC21CCN(C(=O)OC(C)(C)C)CC1 ; COc1c(F)cccc1Nc1c(-c2ccncn2)[nH]c2c1C(=O)NCC21CCN(C(=O)OC(C)(C)C)CC1]

A mixture of tert-butyl 3′-[(3-fluoro-2-methoxyphenyl)amino]-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (800 mg, 1.407 mmol, 1 equiv) and Raney Nickel (180.79 mg, 2.111 mmol, 1.5 equiv) in EtOH (16 mL) was stirred for 2 h at 80° C. under nitrogen atmosphere. The resulting mixture was filtered, the filter cake was washed with EtOAc. The filtrate was concentrated under reduced pressure. The resulting mixture was concentrated under reduced pressure. The crude product mixture was used in the next step directly without further purification. This resulted in tert-butyl 3′-[(3-fluoro-2-methoxyphenyl)amino]-4′-oxo-2′-(pyrimidin-4-yl)-5′,6′-dihydro-1′H-spiro[piperidine-4,7′-pyrrolo[3,2-c]pyridine]-1-carboxylate (670 mg, 91.14%) as a brown solid.

LC-MS: (M+H)⁺ found: 523.

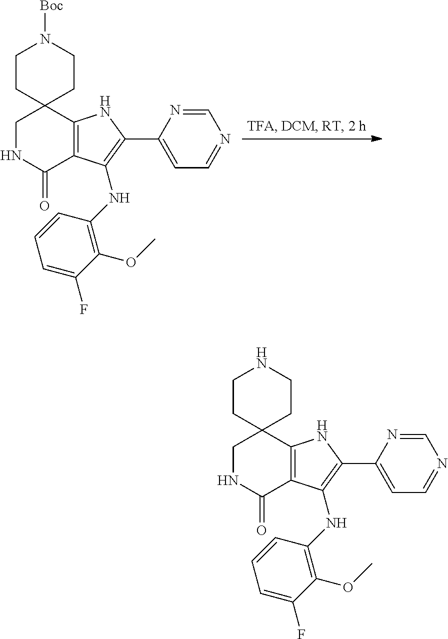 [SMILES: COc1c(F)cccc1Nc1c(-c2ccncn2)[nH]c2c1C(=O)NCC21CCN(C(=O)OC(C)(C)C)CC1 ; COc1c(F)cccc1Nc1c(-c2ccncn2)[nH]c2c1C(=O)NCC21CCNCC1]

A mixture of tert-butyl 3′-[(3-fluoro-2-methoxyphenyl)amino]-4′-oxo-2′-(pyrimidin-4-yl)-5′,6′-dihydro-1′H-spiro[piperidine-4,7′-pyrrolo[3,2-c]pyridine]-1-carboxylate (800 mg, 1.531 mmol, 1 equiv) and TFA (1.75 g, 15.310 mmol, 10 equiv) in DCM (16 mL) was stirred for 2 h at room temperature under air atmosphere. The resulting mixture was concentrated under reduced pressure. The mixture was basified to pH 8 with saturated NaHCO₃ (aq.). The resulting mixture was extracted with CH₂Cl₂. The combined organic layers were dried over anhydrous Na₂SO₄. After filtration, the filtrate was concentrated under reduced pressure. This resulted in 3′-[(3-fluoro-2-methoxyphenyl)amino]-2′-(pyrimidin-4-yl)-5′,6′-dihydro-1′H-spiro[piperidine-4,7′-pyrrolo[3,2-c]pyridin]-4′-one (500 mg, 77.31%) as a brown solid.

LC-MS: (M+H)⁺ found: 423.

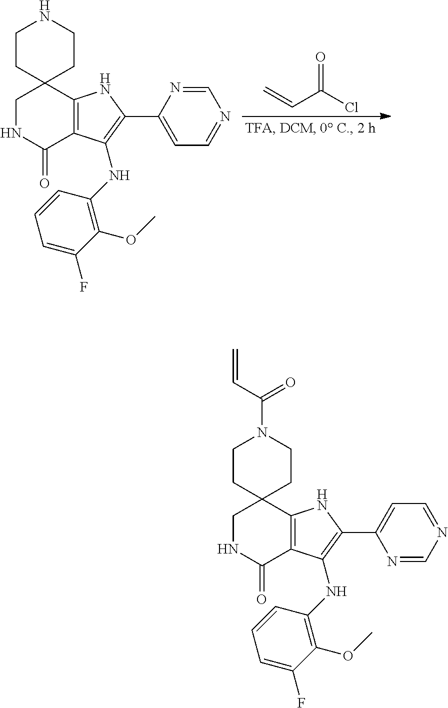

To a stirred mixture of 3′-[(3-fluoro-2-methoxyphenyl)amino]-2′-(pyrimidin-4-yl)-5′,6′-dihydro-1′H-spiro[piperidine-4,7′-pyrrolo[3,2-c]pyridin]-4′-one (300 mg, 0.710 mmol, 1 equiv) and TEA (215.57 mg, 2.130 mmol, 3 equiv) in DCM (6 mL) was added acryloyl chloride (70.70 mg, 0.781 mmol, 1.1 equiv) dropwise at 0° C. under nitrogen atmosphere. The resulting mixture was stirred for 1 h at room temperature under nitrogen atmosphere. The reaction was quenched with Water/Ice at 0° C. The resulting mixture was extracted with CH₂Cl₂. The combined organic layers were washed with brine, dried over anhydrous Na₂SO₄. After filtration, the filtrate was concentrated under reduced pressure. The crude product (mg) was purified by Prep-HPLC with the following conditions to afford 3′-[(3-fluoro-2-methoxyphenyl)amino]-1-(prop-2-enoyl)-2′-(pyrimidin-4-yl)-5′,6′-dihydro-1′H-spiro[piperidine-4,7′-pyrrolo[3,2-c]pyridin]-4′-one (110 mg, 32.51%) as a yellow solid.

LC-MS: (M+H)⁺ found: 477.

¹H NMR (300 MHz, DMSO-d6) δ 11.82 (s, 1H), 9.03 (d, J=1.4 Hz, 1H), 8.56 (d, J=5.6 Hz, 1H), 8.31 (s, 1H), 7.36 (d, J=3.0 Hz, 1H), 7.29 (dd, J=5.6, 1.4 Hz, 1H), 6.87 (dd, J=16.7, 10.4 Hz, 1H), 6.77 (td, J=8.3, 6.0 Hz, 1H), 6.60 (ddd, J=11.0, 8.3, 1.4 Hz, 1H), 6.22 (dd, J=8.3, 1.4 Hz, 1H), 6.16 (dd, J=16.7, 2.6 Hz, 1H), 5.72 (dd, J=10.4, 2.5 Hz, 1H), 4.45 (d, J=13.2 Hz, 1H), 4.06 (d, J=14.1 Hz, 1H), 3.95 (d, J=0.9 Hz, 3H), 3.52 (d, J=2.9 Hz, 2H), 3.26 (t, J=13.3 Hz, 1H), 2.81 (t, J=13.1 Hz, 1H), 2.14 (d, J=8.4 Hz, 2H), 1.71 (d, J=13.2 Hz, 2H).

### Example 47. 3-[(3-fluoro-2-methoxyphenyl)amino]-2-[5-(2-methoxyethoxy)pyrimidin-4-yl]-1H,5H,6H,7H-pyrrolo[3,2-c]pyridin-4-one (compound 676)

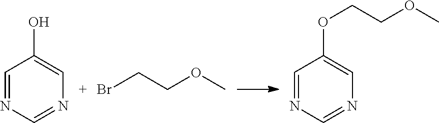

To a stirred mixture of pyrimidin-5-ol (2.00 g, 20.814 mmol, 1.00 equiv) and 2-bromoethyl methyl ether (3.47 g, 0.025 mmol, 1.2 equiv) in DMF (15.00 mL) was added K₂CO₃ (5.75 g, 0.042 mmol, 2.0 equiv) at rt under N2 atmosphere. Then, the solution was stirred at 60 degrees C. for 3 h. The resulting mixture was diluted with water (50 mL) and extracted with 6×60 mL of EA. The filtrate was concentrated under reduced pressure. The residue was purified by reverse flash chromatography with the following conditions: column, C18 silica gel; mobile phase, MeOH in DCM, 0% to 5% gradient in 10 min; detector, UV 254 nm to afford 5-(2-methoxyethoxy) pyrimidine (0.848 g, 26.4%) as a yellow oil.

LC-MS: M+H found: 155.1.

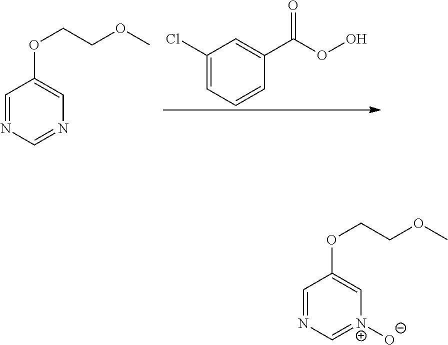

To a stirred solution of 5-(2-methoxyethoxy) pyrimidine (848.00 mg, 5.500 mmol, 1.00 equiv) in CHCl3 (8.00 mL) were added m-CPBA (1423.74 mg, 8.251 mmol, 1.5 equiv) at rt under N2 atmosphere. The solution was stirred at rt for 1 h first, and then stirred at 60 degrees C. for 1.5 h. The resulting mixture was diluted with saturated NaHCO₃ (30 mL) and extracted with 3×20 mL of EA. The filtrate was concentrated under reduced pressure. The residue was purified by reverse flash chromatography with the following conditions: column, C18 silica gel; mobile phase, MeOH in DCM, 0% to 10% gradient in 10 CV: detector, UV 254 nm to afford 5-(2-methoxyethoxy) pyrimidin-1-ium-1-olate (400 mg, 43.2%) as a white solid.

LC-MS: M+H found: 171.0.

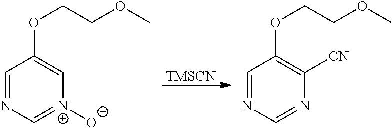

To a stirred solution of 5-(2-methoxyethoxy) pyrimidin-1-ium-1-olate (1.26 g, 7.404 mmol, 1.00 equiv) and Et3N (1.51 g, 0.015 mmol, 2.00 equiv) in ACN (6.00 mL) was added trimethylsilyl cyanide (0.73 g, 7.404 mmol, 1.00 equiv) at rt. The solution was stirred at rt for 16 h. The solution was concentrated under reduced pressure. The residue was purified by reverse flash chromatography with the following conditions: column, C18 silica gel; mobile phase, MeOH in DCM 0% to 10% gradient in 10 CV; detector, UV 254 nm to afford the surplus of 5-(2-methoxyethoxy)pyrimidine-4-carbonitrile (523 mg, 39.42%) as a yellow oil.

LC-MS: M+H found: 180.15.

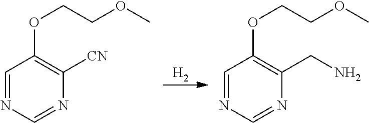

Into a 100 mL stand-up flask were added 5-(2-methoxyethoxy) pyrimidine-4-carbonitrile (523.00 mg, 2.919 mmol, 1.00 equiv), Raney Ni (55.00 mg, 0.642 mmol, 0.22 equiv) and NH3 (g) in MeOH (15.00 mL) at rt. Then, the resulting solution was backfilled with the hydrogen. The solution was stirred at rt for 16 h. The resulting mixture was filtered, the filtrate was concentrated under reduced pressure to afford 1-[5-(2-methoxyethoxy) pyrimidin-4-yl] methanamine (534 mg, 100%) as a blue oil.

LC-MS: M+H found: 171.05.

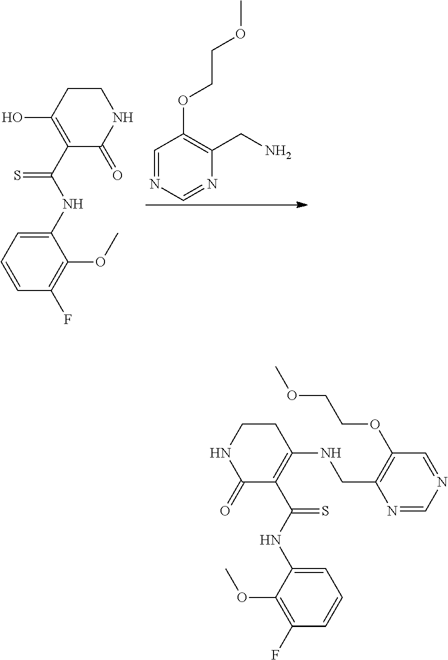

To a stirred solution of 1-[5-(2-methoxyethoxy) pyrimidin-4-yl] methanamine (534.00 mg, 2.915 mmol, 1.00 equiv) and N-(3-fluoro-2-methoxyphenyl)-4-hydroxy-2-oxo-5,6-dihydro-1 H-pyridine-3-carbothioamide (863.68 mg, 2.915 mmol, 1.0 equiv) in DMA (15.00 mL) were added 4A molecular sieves (640.00 mg) at rt under N2 atmosphere. Then, the solution was stirred at 120 degrees C. for 2 h. The resulting mixture was diluted with water (50 mL) and washed with EA (4×50 mL). Then, the organic layer was concentrated under reduced pressure. The residue was purified by reverse flash chromatography with the following conditions: column, C18 silica gel; mobile phase, MeOH in DCM, 0% to 10% gradient in 10 CV; detector, UV 254 nm to afford N-(3-fluoro-2-methoxyphenyl)-4-([[5-(2-methoxyethoxy) pyrimidin-4-yl] methyl]amino)-2-oxo-5,6-dihydro-1H-pyridine-3-carbothioamide (299 mg, 22.2%) as a yellow solid.

LC-MS: M+H found: 462.15.

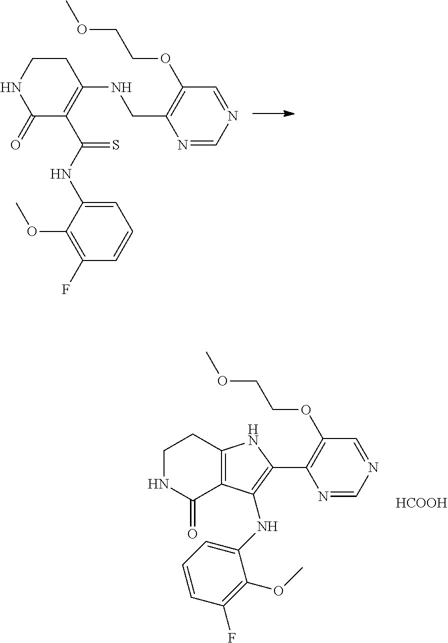 [SMILES: COCCOc1cncnc1CNC1=C(C(=S)Nc2cccc(F)c2OC)C(=O)NCC1 ; COCCOc1cncnc1-c1[nH]c2c(c1Nc1cccc(F)c1OC)C(=O)NCC2 ; "HCOOH"]

To a stirred solution of N-(3-fluoro-2-methoxyphenyl)-4-([[5-(2-methoxyethoxy)pyrimidin-4-yl]methyl]amino)-2-oxo-5,6-dihydro-1H-pyridine-3-carbothioamide (220.00 mg, 0.477 mmol, 1.00 equiv) and H₂O₂ (30%) (81.10 mg, 2.384 mmol, 5.00 equiv) in DMSO (13.00 mL) were added TFA (59.80 mg, 0.524 mmol, 1.10 equiv) at rt under N₂ atmosphere. Then, the solution was stirred at 80 degrees C. for 2 h. The resulting mixture was diluted with water (60 mL) and extracted with 4×60 mL of EA. The filtrate was concentrated under reduced pressure. The residue was purified by re verse flash chromatography with the following conditions: Column: XBridge Prep OBD C18 Column, 30*150 mm, 5 μm; Mobile Phase A: Water (0.1% FA), Mobile Phase B: ACN; Flow rate: 60 mL/min; Gradient: 25% B to 35% B in 8 min; Wave Length: 254 nm; RT1 (min): 6; to afford 3-[(3-fluoro-2-methoxyphenyl)amino]-2-[5-(2-methoxyethoxy)pyrimidin-4-yl]-1H,5H,6H,7H-pyrrolo[3,2-c]pyridin-4-one (19.0 mg, 9.04%) as a yellow solid.

LC-MS: (M+H)⁺ found 428.15.

¹H NMR (300 MHz, DMSO-d6) δ 11.11 (s, 1H), 9.12 (s, 1H), 8.70 (s, 1H), 8.51 (s, 1H), 7.16 (s, 1H), 6.73 (td, J=8.3, 6.0 Hz, 1H), 6.55 (ddd, J=11.1, 8.3, 1.5 Hz, 1H), 6.41-6.32 (m, 1H), 4.33-4.24 (m, 2H), 3.94 (d, J=1.0 Hz, 3H), 3.84-3.69 (m, 2H), 3.45 (q, J=4.5 Hz, 2H), 2.89 (t, J=6.7 Hz, 2H).

### Example 48. (7R)-2-(2-aminopyrimidin-4-yl)-3-[(3-chloro-2-methoxyphenyl)amino]-7-(2-methoxyethyl)-1H,5H,6H,7H-pyrrolo[3,2-c]pyridin-4 one (compound 726)

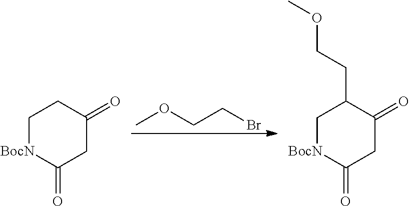

To a stirred solution of tert-butyl 2,4-dioxopiperidine-1-carboxylate (5.00 g, 23.449 mmol, 1.00 equiv) and 2-bromoethyl methyl ether (8.15 g, 58.623 mmol, 2.50 equiv) in THF (20.00 mL) were added LiHMDS (70.00 mL, 418.345 mmol, 17.84 equiv) at −20 degrees C. under N2 atmosphere. Then, the solution was stirred at −20 degrees C. for 1 h. The mixture was diluted with water (50 ml) and added 2M HCl to adjust PH to 4, then extracted with 3×40 ml of EA. The filtrate was concentrated under reduced pressure to afford crude 5-(2-methoxyethyl)piperidine-2,4-dione (6.0 g, 149.47%) as a yellow oil.

LC-MS: M−57 found: 216.2.

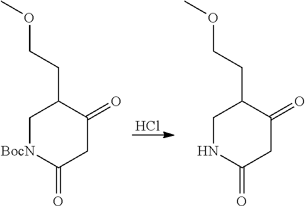 [SMILES: COCCC1CN(C(=O)OC(C)(C)C)C(=O)CC1=O ; Cl ; COCCC1CNC(=O)CC1=O]

To a stirred solution of tert-butyl 5-(2-methoxyethyl)-2,4-dioxopiperidine-1-carboxylate (3.00 mg, 0.011 mmol, 1.00 equiv) in DCM (15.00 mL) were added HCl (gas) in 1,4-dioxane (15.00 mg, 0.219 mmol, 19.80 equiv) at 0 degrees C. under N2 atmosphere. Then, the solution was stirred at rt for 2 h. The resulting mixture was concentrate under reduced pressure to afford about 3.2 g of crude product as a yellow oil. LC-MS: M+CH₃ found: 186.1.

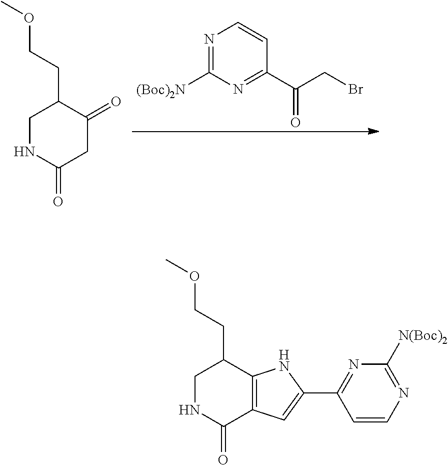

To a stirred solution of 5-(2-methoxyethyl)piperidine-2,4-dione (1.90 mg, 0.011 mmol, 1.00 equiv) and NH₄OAc (5.13 mg, 0.066 mmol, 6.00 equiv) in EtOH (15.00 mL) were added tert-butyl N-[4-(2-bromoacetyl)pyrimidin-2-yl]-N-(tert-butoxycarbonyl)carbamate (4.62 mg, 0.011 mmol, 1.00 equiv) at rt under N2 atmosphere. Then, the solution was stirred at 50 degrees C. for 2 h. The resulting mixture was diluted with water (50 ml), some solid was get by filtrated to afford crude product about 2.6 g as a yellow solid.

LC-MS: M+H found: 488.15.

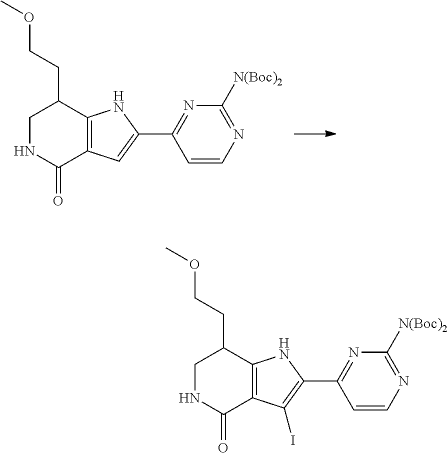

To a stirred solution of tert-butyl N-(tert-butoxycarbonyl)-N-[4-[7-(2-methoxyethyl)-4-oxo-1H,5H,6H,7H-pyrrolo[3,2-c]pyridin-2-yl]pyrimidin-2-yl]carbamate (1500.00 mg, 3.077 mmol, 1.00 equiv) in DMF (30.00 mL) were added NIS (969.05 mg, 4.307 mmol, 1.40 equiv) at rt under N2 atmosphere. The solution was stirred at rt for 16 h. The resulting mixture was diluted with water (80 ml) and extracted with 2×70 ml of EA. The combined organic layer was concentrated under reduced pressure. The residue was purified by reverse flash chromatography with the following conditions: column, C18 silica gel; mobile phase, MeOH in DCM, 0% to 7% gradient in 10 min; detector, UV 254 nm to afford tert-butyl N-(tert-butoxycarbonyl)-N-[4-[3-iodo-7-(2-methoxyethyl)-4-oxo-1H,5H,6H,7H-pyrrolo[3,2-c]pyridin-2-yl]pyrimidin-2-yl]carbamate (650 mg, 34.44%) as a yellow oil.

LC-MS: M+H found: 614.05.

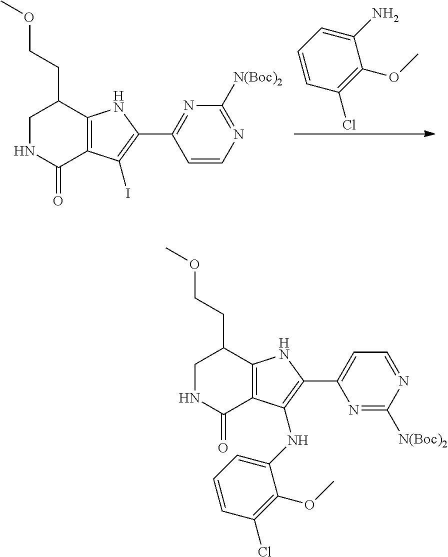

To a stirred solution of tert-butyl N-(tert-butoxycarbonyl)-N-[4-[3-iodo-7-(2-methoxyethyl)-4-oxo-1H,5H,6H,7H-pyrrolo[3,2-c]pyridin-2-yl]pyrimidin-2-yl]carbamate (650.00 mg, 1.060 mmol, 1.00 equiv) and 3-chloro-2-methoxyaniline (250.48 mg, 1.589 mmol, 1.50 equiv) in dioxane (20.00 mL) were added Cs₂CO₃ (690.46 mg, 2.119 mmol, 2.00 equiv) and EPhos Pd G4 (145.99 mg, 0.159 mmol, 0.15 equiv) at rt under N2 atmosphere. Then, the solution was stirred at 50 degrees C. for 2 h. The resulting mixture was concentrated under reduced pressure. The residue was purified by Prep-TLC (DCM:MeOH=20:1) to afford tert-butyl N-(tert-butoxycarbonyl)-N-(4-[3-[(3-chloro-2-methoxyphenyl)amino]-7-(2-methoxyethyl)-4-oxo-1H,5H,6H,7H-pyrrolo[3,2-c]pyridin-2-yl]pyrimidin-2-yl)carbamate (184 mg, 27.00%) as a yellow oil.

LC-MS: M−53 found: 589.1.

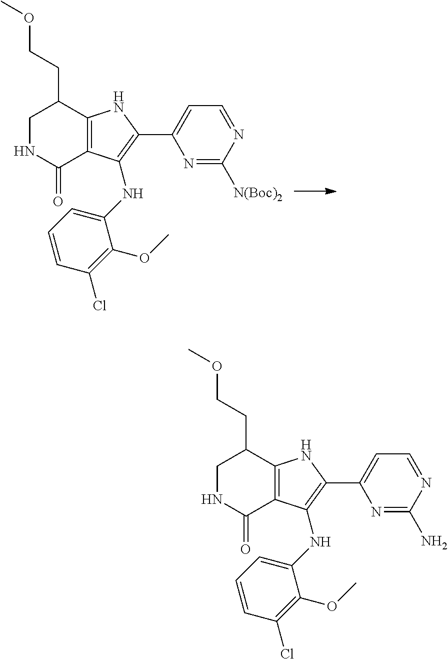

To a stirred solution of tert-butyl 4-[(3-chloro-2-methoxyphenyl)carbonothioyl]-3-([[3-(2-methoxy-2-methylpropoxy)pyridin-4-yl]methyl]amino)-5-oxo-2,6-dihydropyridine-1-carboxylate (100.00 mg, 0.165 mmol, 1.00 equiv) and H₂O₂ (11.24 mg, 0.330 mmol, 2.00 equiv) in EtOH (2.5. mL) were added TFA (20.73 mg, 0.182 mmol, 1.10 equiv) at rt under N₂ atmosphere. Then, the solution was stirred at 80 degrees C. for 2 h. The resulting mixture was diluted with water (20 ml) and extracted with EA (3×20 ml). Then, the organic layer was concentrated under reduced pressure. The crude product (mg) was purified by Prep-HPLC with the following conditions Column: CHIRAL ART Cellulose-SC, 2*25 cm, 5 μm; Mobile Phase A: Hex:DCM=3:1 (0.5% 2M NH3-MeOH), Mobile Phase B: IPA; Flow rate: 20 mL/min; Gradient: 20% B to 20% B in 14 min; Wave Length: 220/254 nm; RT1 (min): 8.26; RT2 (min): 11.54; to afford tert-butyl 3-[(3-chloro-2-methoxyphenyl)amino]-2-[3-(2-methoxy-2-methylpropoxy)pyridin-4-yl]-4-oxo-1H,5H,7H-pyrrolo[2,3-c]pyridine-6-carboxylate (22.7 mg) as a yellow solid.

LC-MS: (M+H)⁺ found 443.2.

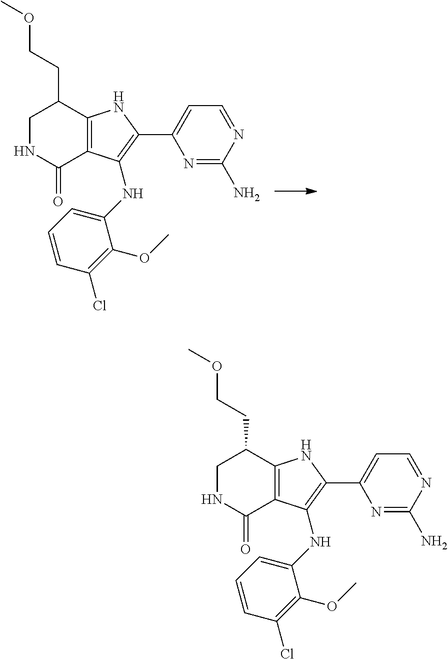 [SMILES: COCCC1CNC(=O)c2c1[nH]c(-c1ccnc(N)n1)c2Nc1cccc(Cl)c1OC ; COCC[C@H]1CNC(=O)c2c1[nH]c(-c1ccnc(N)n1)c2Nc1cccc(Cl)c1OC]

The 2-(2-aminopyrimidin-4-yl)-3-[(3-chloro-2-methoxyphenyl)amino]-7-(2-methoxyethyl)-1H,5H,6H,7H-pyrrolo[3,2-c]pyridin-4-one (213.00 mg) was split into (7R)-2-(2-aminopyrimidin-4-yl)-3-[(3-chloro-2-methoxyphenyl)amino]-7-(2-methoxyethyl)-1H,5H,6H,7H-pyrrolo[3,2-c]pyridin-4 one (44.5 mg, 20.89%) as a yellow solid.

LC-MS: (M+H)⁺ found 443.2.

¹H NMR (300 MHz, DMSO-d6) δ 11.46 (s, 1H), 8.07 (d, J=5.3 Hz, 1H), 7.98 (s, 1H), 7.12 (s, 1H), 6.88-6.72 (m, 2H), 6.50 (d, J=5.3 Hz, 1H), 6.36 (dd, J=7.8, 1.9 Hz, 1H), 6.21 (s, 2H), 3.90 (s, 3H), 3.50 (d, J=5.7 Hz, 3H), 3.35 (s, 3H), 3.23 (m, 1H), 3.07 (M, 1H), 2.04-1.93 (m, 1H), 1.77 (dd, J=13.7, 7.7 Hz, 1H).

### Example 49. (7S)-2-(2-aminopyrimidin-4-yl)-3-[(3-chloro-2-methoxyphenyl)amino]-7-(2-methoxyethyl)-1H,5H,6H,7H-pyrrolo[3,2-c]pyridin-4-one (compound 727)

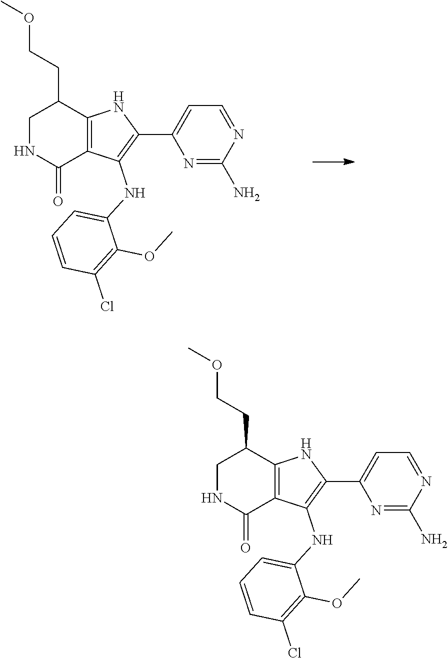 [SMILES: COCCC1CNC(=O)c2c1[nH]c(-c1ccnc(N)n1)c2Nc1cccc(Cl)c1OC ; COCC[C@@H]1CNC(=O)c2c1[nH]c(-c1ccnc(N)n1)c2Nc1cccc(Cl)c1OC]

The 2-(2-aminopyrimidin-4-yl)-3-[(3-chloro-2-methoxyphenyl)amino]-7-(2-methoxyethyl)-1H,5H,6H,7H-pyrrolo[3,2-c]pyridin-4-one (213.00 mg) was split (HPLC conditions: Column: CHIRAL ART Cellulose-SC, 2*25 cm, 5 m; Mobile Phase A: Hex: DCM=3:1 (0.5% 2M NH3-MeOH), Mobile Phase B: IPA; Flow rate: 20 mL/min; Gradient: 20% B to 20% B in 14 min; Wave Length: 220/254 nm; RT1 (min): 8.26; RT2 (min): 11.54 [symbol] into (7S)-2-(2-aminopyrimidin-4-yl)-3-[(3-chloro-2-methoxyphenyl)amino]-7-(2-methoxyethyl)-1H,5H,6H,7H-pyrrolo[3,2-c]pyridin-4-one (34.4 mg, 16.15%) as a yellow solid.

LC-MS: (M+H)⁺ found 443.2.

¹H NMR (300 MHz, DMSO-d6) δ 11.46 (s, 1H), 8.07 (d, J=5.3 Hz, 1H), 7.98 (s, 1H), 7.12 (s, 1H), 6.88-6.72 (m, 2H), 6.50 (d, J=5.3 Hz, 1H), 6.36 (dd, J=7.8, 1.9 Hz, 1H), 6.21 (s, 2H), 3.90 (s, 3H), 3.50 (d, J=5.7 Hz, 3H), 3.35 (s, 3H), 3.23 (m, 1H), 3.07 (M, 1H), 2.04-1.93 (m, 1H), 1.77 (dd, J=13.7, 7.7 Hz, 1H).

### Example 50. (7S)-3-[(3-chloro-2-methoxyphenyl)amino]-2-(6-methoxy-1,5-naphthyridin-4-yl)-7-(methoxymethyl)-1H,5H,6H,7H-pyrrolo[3,2-c]pyridin-4-one (compound 765)

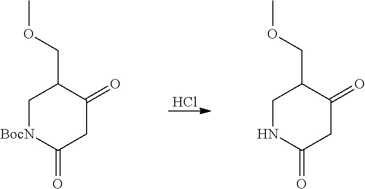 [SMILES: COCC1CN(C(=O)OC(C)(C)C)C(=O)CC1=O ; Cl ; COCC1CNC(=O)CC1=O]

To a stirred solution of tert-butyl 5-(methoxymethyl)-2,4-dioxopiperidine-1-carboxylate (1.00 g, 3.887 mmol, 1.00 equiv) in DCM (5.00 mL) was added HCl (gas) in 1,4-dioxane (5.00 mL) at rt. The solution was stirred at rt about 2 h. The resulting mixture was concentrated under reduced pressure to obtain 5-(methoxymethyl)piperidine-2,4-dione (1.04 g, 170.25%) as a yellow oil.

LC-MS: M+H found: 158.10.

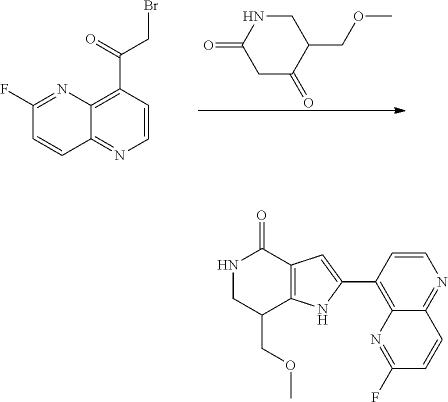 [SMILES: O=C(CBr)c1ccnc2ccc(F)nc12 ; COCC1CNC(=O)CC1=O ; COCC1CNC(=O)c2cc(-c3ccnc4ccc(F)nc34)[nH]c21]

To a stirred solution of 5-(methoxymethyl)piperidine-2,4-dione (611.00 mg, 3.888 mmol, 1.00 equiv) and 2-bromo-1-(6-fluoro-1,5-naphthyridin-4-yl)ethanone (1046.03 mg, 3.888 mmol, 1.00 equiv) in EtOH (10.00 mL) were added NH₄OAc (1498.30 mg, 19.438 mmol, 5.00 equiv) at rt. Then the solution was stirred at 50 degrees C. about 16 h. The resulting mixture was diluted with water (200 mL) and was filtered, and the filter cake was washed with 50 mL of EA. After filtration, the filtrate was concentrated under reduced pressure to obtain 22-(6-fluoro-1,5-naphthyridin-4-yl)-7-(methoxymethyl)-1H,5H,6H,7H-pyrrolo[3,2-c]pyridin-4-one (474.7 mg, 37.42%) as a red solid.

LC-MS: M+H found: 327.05.

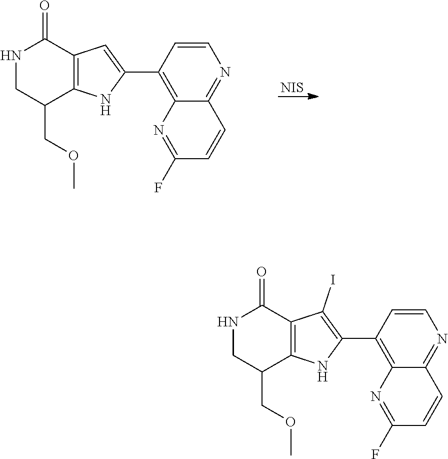

To a stirred solution of 2-(6-fluoro-1,5-naphthyridin-4-yl)-7-(methoxymethyl)-1H,5H,6H,7H-pyrrolo[3,2-c]pyridin-4-one (348.40 mg, 1.068 mmol, 1.00 equiv) in DMF (12.00 mL) and was added NIS (360.30 mg, 1.601 mmol, 1.50 equiv) at rt. The solution was stirred at it about 16 h. The resulting mixture was diluted with water (100 mL) and filtered. The filterate was concentrated under reduced pressure to obtain 2-(6-fluoro-1,5-naphthyridin-4-yl)-3-iodo-7-(methoxymethyl)-1H,5H,6H,7H-pyrrolo[3,2-c]pyridin-4-one (392.6 mg, 81.32%) as a red solid.

LC-MS: M+H found: 453.05.

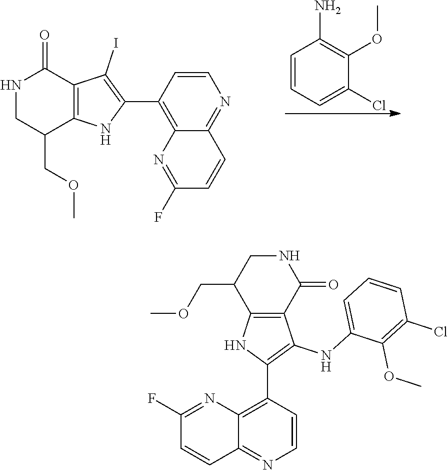

To a stirred solution of 2-(6-fluoro-1,5-naphthyridin-4-yl)-3-iodo-7-(methoxymethyl)-1H,5H,6H,7H-pyrrolo[3,2-c]pyridin-4-one (100.00 mg, 0.221 mmol, 1.00 equiv) and 3-chloro-2-methoxyaniline (52.27 mg, 0.332 mmol, 1.50 equiv) in dioxane (6.00 mL, 70.825 mmol, 320.29 equiv) in DMF (2.00 mL) were added Cs₂CO₃ (144.10 mg, 0.442 mmol, 2.00 equiv), and EPhos Pd G4 (40.62 mg, 0.044 mmol, 0.20 equiv) at rt. The solution was stirred at 50 degrees C. about 16 h. The resulting mixture was diluted with water (50 mL) and washed with 2×40 mL of EA. The combined organic layers were washed with saturated salt solution (2×40 mL), dried over anhydrous sodium sulfate. After filtration, the filtrate was concentrated under reduced pressure. The residue was purified by Prep-TLC (MeOH:DCM=30:1) to afford 3-[(3-chloro-2-methoxyphenyl)amino]-2-(6-fluoro-1,5-naphthyridin-4-yl)-7-(methoxymethyl)-1H,5H,6H,7H-pyrrolo[3,2-c]pyridin-4-one (85 mg, 79.76%) as a yellow solid.

LC-MS: M+H found: 482.15.

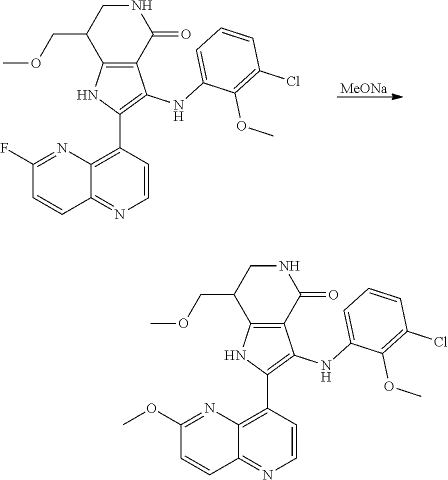 [SMILES: "MeONa" ; COCC1CNC(=O)c2c1[nH]c(-c1ccnc3ccc(F)nc13)c2Nc1cccc(Cl)c1OC ; COCC1CNC(=O)c2c1[nH]c(-c1ccnc3ccc(OC)nc13)c2Nc1cccc(Cl)c1OC]

To a stirred solution 3-[(3-chloro-2-methoxyphenyl)amino]-2-(6-fluoro-1,5-naphthyridin-4-yl)-7-(methoxymethyl)-1H,5H,6H,7H-pyrrolo[3,2-c]pyridin-4-one (80.00 mg, 0.166 mmol, 1.00 equiv) in MeOH (4.00 mL) was added MeONa (10.76 mg, 0.199 mmol, 1.20 equiv) at rt. The solution was stirred at 80 degrees C. under N2 atmosphere about 1 h. The resulting mixture was diluted with water (50 mL) and was washed with 3×40 mL of EA. The combined organic layers were washed with saturated salt solution (2×40 mL), dried over anhydrous sodium sulfate. After filtration, the filtrate was concentrated under reduced pressure to obtain 3-[(3-chloro-2-methoxyphenyl)amino]-2-(6-methoxy-1,5-naphthyridin-4-yl)-7-(methoxymethyl)-1H,5H,6H,7H-pyrrolo[3,2-c]pyridin-4-one (58.8 mg, 71.71%) as a yellow solid.

LC-MS: M+H found: 494.2.

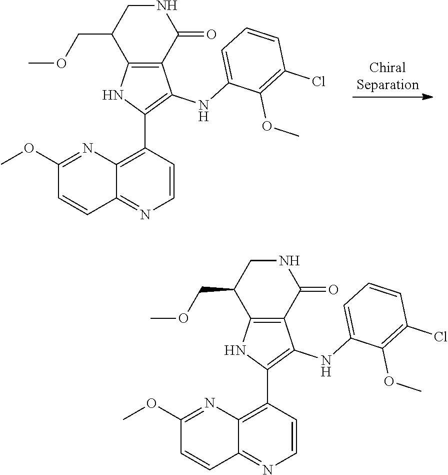 [SMILES: COCC1CNC(=O)c2c1[nH]c(-c1ccnc3ccc(OC)nc13)c2Nc1cccc(Cl)c1OC ; COC[C@H]1CNC(=O)c2c1[nH]c(-c1ccnc3ccc(OC)nc13)c2Nc1cccc(Cl)c1OC]

The 3-[(3-chloro-2-methoxyphenyl)amino]-2-(6-methoxy-1,5-naphthyridin-4-yl)-7-(methoxymethyl)-1H,5H,6H,7H-pyrrolo[3,2-c]pyridin-4-one (30 mg, 0.061 mmol, 1.00 equiv) was split into (7S)-3-[(3-chloro-2-methoxyphenyl)amino]-2-(6-methoxy-1,5-naphthyridin-4-yl)-7-(methoxymethyl)-1H,5H,6H,7H-pyrrolo[3,2-c]pyridin-4-one (10.8 mg, 36.00%) as a yellow solid.

LC-MS: M+H found 494.0.

¹H NMR (400 MHz, DMSO-d6) δ 12.12 (s, 1H), 8.58 (d, J=4.8 Hz, 1H), 8.31 (d, J=9.1 Hz, 1H), 7.80 (s, 1H), 7.52 (d, J=4.8 Hz, 1H), 7.37 (d, J=9.1 Hz, 1H), 7.19 (s, 1H), 6.77-6.62 (m, 2H), 6.17 (dd, J=7.7, 2.0 Hz, 1H), 4.20 (s, 3H), 3.89 (s, 3H), 3.62 (d, J=6.5 Hz, 2H), 3.56-3.49 (m, 1H), 3.38 (d, J=10.1 Hz, 2H), 2.68 (s, 3H).

### Example 51. (7S)-3-[(3-chloro-2-methoxyphenyl)amino]-2-(6-methoxy-1,5-naphthyridin-4-yl)-7-(methoxymethyl)-1H,5H,6H,7H-pyrrolo[3,2-c]pyridin-4-one (compound 764)

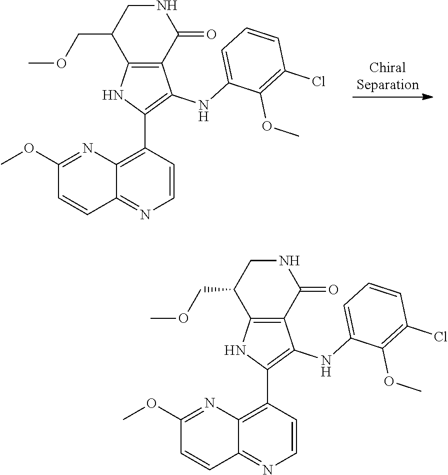 [SMILES: COCC1CNC(=O)c2c1[nH]c(-c1ccnc3ccc(OC)nc13)c2Nc1cccc(Cl)c1OC ; COC[C@@H]1CNC(=O)c2c1[nH]c(-c1ccnc3ccc(OC)nc13)c2Nc1cccc(Cl)c1OC]

The 3-[(3-chloro-2-methoxyphenyl)amino]-2-(6-methoxy-1,5-naphthyridin-4-yl)-7-(methoxymethyl)-1H,5H,6H,7H-pyrrolo[3,2-c]pyridin-4-one (30 mg, 0.061 mmol, 1 equiv) was split into (7S)-3-[(3-chloro-2-methoxyphenyl)amino]-2-(6-methoxy-1,5-naphthyridin-4-yl)-7-(methoxymethyl)-1H,5H,6H,7H-pyrrolo[3,2-c]pyridin-4-one (7.8 mg, 26.00%) as a yellow solid.

LC-MS: M+H found 494.0.

¹H NMR (400 MHz, DMSO-d6) δ 12.12 (s, 1H), 8.58 (d, J=4.8 Hz, 1H), 8.31 (d, J=9.1 Hz, 1H), 7.80 (s, 1H), 7.52 (d, J=4.8 Hz, 1H), 7.37 (d, J=9.1 Hz, 1H), 7.19 (s, 1H), 6.77-6.62 (m, 2H), 6.17 (dd, J=7.7, 2.0 Hz, 1H), 4.20 (s, 3H), 3.89 (s, 3H), 3.62 (d, J=6.5 Hz, 2H), 3.56-3.49 (m, 1H), 3.38 (d, J=10.1 Hz, 2H), 2.68 (s, 3H).

### Example 52. (R)-3-((3-chloro-2-methoxyphenyl)amino)-2-(6-cyclopropoxy-1,5-naphthyridin-4-yl)-7-methyl-1,5,6,7-tetrahydro-4H-pyrrolo[3,2-c]pyridin-4-one (compound 769)

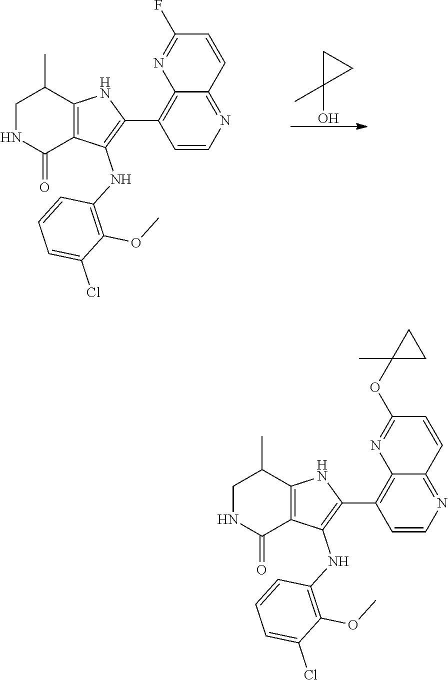

A solution of 3-[(3-chloro-2-methoxyphenyl)amino]-2-(6-fluoro-1,5-naphthyridin-4-yl)-7-methyl-1H,5H,6H,7H-pyrrolo[3,2-c]pyridin-4-one (700.24 mg, 1.550 mmol, 0.9 equiv) in DMF (1.5 mL) was treated with NaH (82.64 mg, 3.444 mmol, 2 equiv) for 30 min at 0° C. under nitrogen atmosphere followed by the addition of 3-[(3-chloro-2-methoxyphenyl)amino]-2-(6-fluoro-1,5-naphthyridin-4-yl)-7-methyl-1H,5H,6H,7H-pyrrolo[3,2-c]pyridin-4-one (700.24 mg, 1.550 mmol, 0.9 equiv) in portions at RT for 2 h. The reaction was ok. The aqueous layer was extracted with EA (3×10 ml). The resulting mixture was concentrated under reduced pressure. The residue was purified by Prep-TLC (DCM/MeOH=10:1) to afford 3-[(3-chloro-2-methoxyphenyl)amino]-2-(6-cyclopropoxy-1,5-naphthyridin-4-yl)-7-methyl-1H,5H,6H,7H-pyrrolo[3,2-c]pyridin-4-one as a yellow solid.

LC-MS: M+H found: 490.

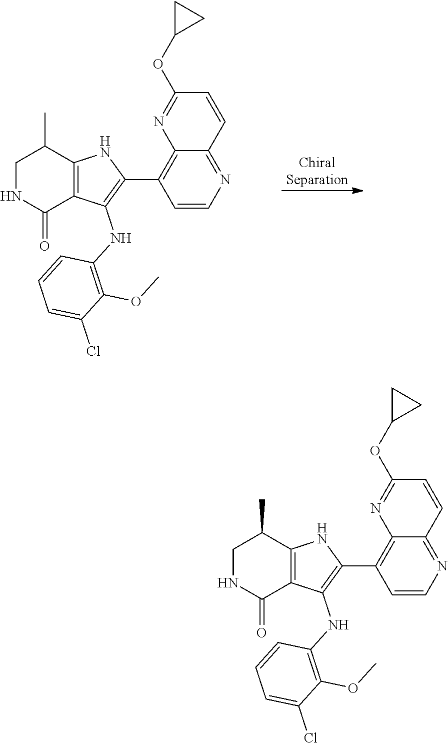 [SMILES: COc1c(Cl)cccc1Nc1c(-c2ccnc3ccc(OC4CC4)nc23)[nH]c2c1C(=O)NCC2C ; COc1c(Cl)cccc1Nc1c(-c2ccnc3ccc(OC4CC4)nc23)[nH]c2c1C(=O)NC[C@H]2C]

The crude product 3-[(3-chloro-2-methoxyphenyl)amino]-2-(6-cyclopropoxy-1,5-naphthyridin-4-yl)-7-methyl-1H,5H,6H,7H-pyrrolo[3,2-c]pyridin-4-one (58.00 mg) was purified by Prep-chiral-HPLC with the following conditions (Column: CHIRALPAK IG-3, 4.6*50 mm, 3 um; Mobile Phase A: (Hex:DCM=3:1)(0.1% DEA):EtOH=50:50; Flow rate: 1 mL/min; Gradient: 0% B to 0% B; Injection Volume: 5 ul mL) fractions containing the desired compound were evaporated to dryness to afford (7R)-3-[(3-chloro-2-methoxyphenyl)amino]-2-(6-cyclopropoxy-1,5-naphthyridin-4-yl)-7-methyl-1H,5H,6H,7H-pyrrolo[3,2-c]pyridin-4-one (12.1 mg) as a yellow solid and (7S)-3-[(3-chloro-2-methoxyphenyl)amino]-2-(6-cyclopropoxy-1,5-naphthyridin-4-yl)-7-methyl-1H,5H,6H,7H-pyrrolo[3,2-c]pyridin-4-one (12.1 mg) as a yellow solid

LC-MS: M+H found: 490.

¹H NMR (400 MHz, DMSO-d6) δ 12.43 (s, 1H), 8.59 (d, J=4.9 Hz, 1H), 8.32 (d, J=9.0 Hz, 1H), 7.87 (s, 1H), 7.52 (d, J=4.9 Hz, 1H), 7.34 (d, J=9.0 Hz, 1H), 7.26 (d, J=2.9 Hz, 1H), 6.79-6.67 (m, 2H), 6.18 (dd, J=7.7, 1.9 Hz, 1H), 4.68-4.59 (m, 1H), 3.92 (s, 3H), 3.57-3.49 (m, 1H), 3.28-3.01 (m, 2H), 1.28 (d, J=6.6 Hz, 3H), 0.98-0.86 (m, 4H).

### Example 53. (S)-3-((3-chloro-2-methoxyphenyl)amino)-2-(6-cyclopropoxy-1,5-naphthyridin-4-yl)-7-methyl-1,5,6,7-tetrahydro-4H-pyrrolo[3,2-c]pyridin-4-one (compound 770)

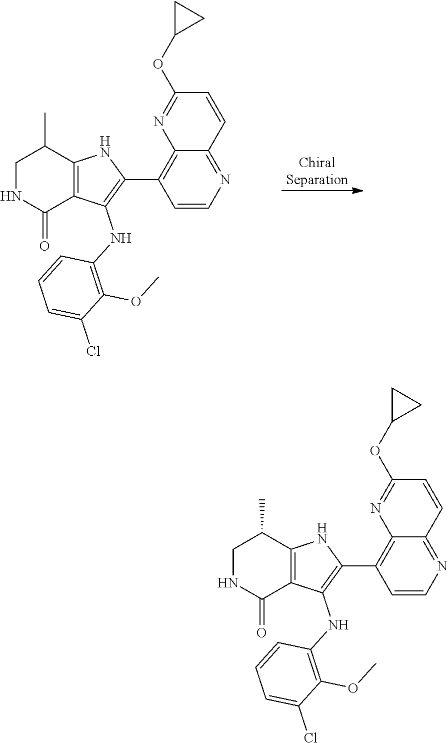 [SMILES: COc1c(Cl)cccc1Nc1c(-c2ccnc3ccc(OC4CC4)nc23)[nH]c2c1C(=O)NCC2C ; COc1c(Cl)cccc1Nc1c(-c2ccnc3ccc(OC4CC4)nc23)[nH]c2c1C(=O)NC[C@@H]2C]

The crude product 3-[(3-chloro-2-methoxyphenyl)amino]-2-(6-cyclopropoxy-1,5-naphthyridin-4-yl)-7-methyl-1H,5H,6H,7H-pyrrolo[3,2-c]pyridin-4-one (58.00 mg) was purified by Prep-chiral-HPLC with the following conditions (Column: CHIRALPAK IG-3, 4.6*50 mm, 3 um; Mobile Phase A: (Hex:DCM=3:1)(0.1% DEA):EtOH=50:50; Flow rate: 1 mL/min; Gradient: 0% B to 0% B; Injection Volume. Sul mL) fractions containing the desired compound were evaporated to dryness to afford (7R)-3-[(3-chloro-2-methoxyphenyl)amino]-2-(6-cyclopropoxy-1,5-naphthyridin-4-yl)-7-methyl-1H,5H,6H,7H-pyrrolo[3,2-c]pyridin-4-one (12.1 mg) as a yellow solid and (7S)-3-[(3-chloro-2-methoxyphenyl)amino]-2-(6-cyclopropoxy-1,5-naphthyridin-4-yl)-7-methyl-1H,5H,6H,7H-pyrrolo[3,2-c]pyridin-4-one (12.1 mg) as a yellow solid

LC-MS: M+H found: 490.

¹H NMR (400 MHz, DMSO-d6) δ 12.43 (s, 1H), 8.59 (d, J=4.9 Hz, 1H), 8.32 (d, J=9.1 Hz, 1H), 7.87 (s, 1H), 7.52 (d, J=4.9 Hz, 1H), 7.34 (d, J=9.1 Hz, 1H), 7.26 (d, J=2.6 Hz, 1H), 6.79-6.67 (m, 2H), 6.18 (dd, J=7.7, 1.9 Hz, 1H), 4.68-4.59 (m, 1H), 3.92 (s, 3H), 3.57-3.48 (m, 1H), 3.25-3.09 (m, 2H), 1.28 (d, J=6.6 Hz, 3H), 0.97-0.88 (m, 4H).

### Example 54. 3-[(3-chloro-2-methoxyphenyl)amino]-2-(6-methoxy-1,7-naphthyridin-4-yl)-1H,5H,6H,7H-pyrrolo[3,2-c]pyridin-4-one (compound 772)

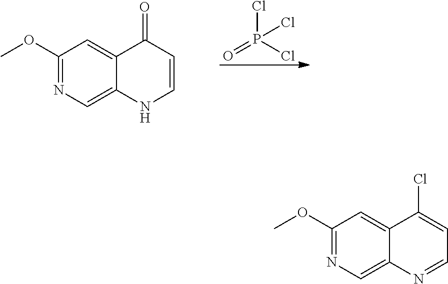

To a stirred mixture of 6-methoxy-1H-1,7-naphthyridin-4-one (400 mg, 2.270 mmol, 1.00 equiv) and phosphorus oxychloride (4 mL, 26.089 mmol, 11.49 equiv) for 2 hours at 100 degrees C. under N₂ atmosphere. The reaction was quenched with H2O at 0 degrees C. The aqueous layer was extracted with EA and H2O 3×1 150 mL). The residue was purified by Prep-TLC (DCM:MeOH=18:1) to afford 4-chloro-6-methoxy-1,7-naphthyridine (420 mg, 95.05%) as a light yellow oil.

LC-MS: M+H found: 194.8.

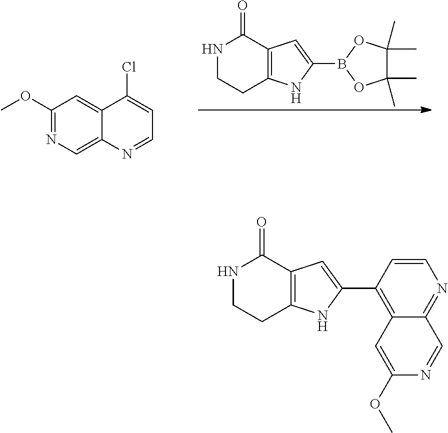 [SMILES: COc1cc2c(Cl)ccnc2cn1 ; CC1(C)OB(c2cc3c([nH]2)CCNC3=O)OC1(C)C ; COc1cc2c(-c3cc4c([nH]3)CCNC4=O)ccnc2cn1]

To a stirred mixture of 4-chloro-6-methoxy-1,7-naphthyridine (60 mg, 0.308 mmol, 1.00 equiv) and 2-(4,4,5,5-tetramethyl-1,3,2-dioxaborolan-2-yl)-1H,5H,6H,7H-pyrrolo[3,2-c]pyridin-4-one (105.05 mg, 0.400 mmol, 1.3 equiv) in dioxane (1.2 mL, 14.165 mmol, 45.95 equiv) and H₂O (0.3 mL, 16.653 mmol, 54.02 equiv) were added XPhos Pd G3 (26.10 mg, 0.031 mmol, 0.1 equiv) and K₂CO₃ (85.22 mg, 0.616 mmol, 2 equiv) in portions at RT. The resulting mixture was stirred for 2 hours at 80 degrees C. under N2 atmosphere. The resulting mixture was filtered, then the filter cake was washed with EA (10 mL). The filtrate was concentrated under reduced pressure. The residue was purified by Prep-TLC (DCM and MeOH 10:1) to afford 2-(6-methoxy-1,7-naphthyridin-4-yl)-1H,5H,6H,7H-pyrrolo[3,2-c]pyridin-4-one (88 mg, 96.99%) as a light yellow solid.

LC-MS: M+H found: 295.0.

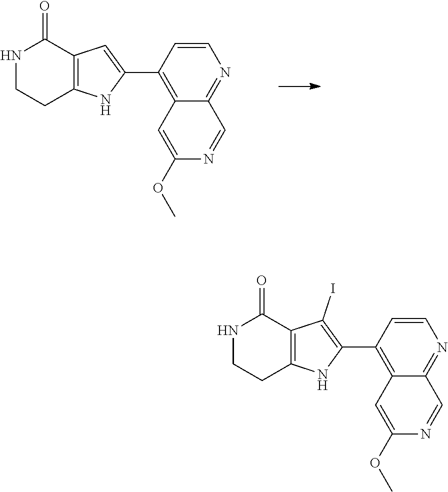 [SMILES: COc1cc2c(-c3cc4c([nH]3)CCNC4=O)ccnc2cn1 ; COc1cc2c(-c3[nH]c4c(c3I)C(=O)NCC4)ccnc2cn1]

To a stirred solution of 2-(6-methoxy-1,7-naphthyridin-4-yl)-1H,5H,6H,7H-pyrrolo[3,2-c]pyridin-4-one (100 mg, 0.340 mmol, 1.00 equiv) in DMF (1 mL, 12.922 mmol, 38.03 equiv) was added NIS (91.73 mg, 0.408 mmol, 1.2 equiv) in portions at RT. The resulting mixture was stirred for 1 hour at RT. The resulting mixture was extracted with EA (3×10 mL). The combined organic layer was concentrated under reduced pressure. The residue was purified by Prep-TLC (DCM and MeOH 15.1) to afford 3-iodo-2-(6-methoxy-1,7-naphthyridin-4-yl)-1H,5H,6H,7H-pyrrolo[3,2-c]pyridin-4-one (96 mg, 67.24%) as a light yellow solid.

LC-MS: M+H found: 420.95.

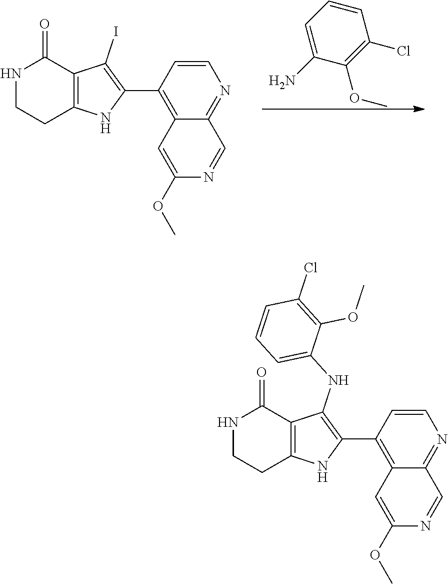

To a stirred mixture of 3-iodo-2-(6-methoxy-1,7-naphthyridin-4-yl)-1H,5H,6H,7H-pyrrolo[3,2-c]pyridin-4-one (20.00 mg, 0.048 mmol, 1.00 equiv) and 3-chloro-2-methoxyaniline (22.50 mg, 0.143 mmol, 3 equiv) in dioxane (0.50 mL) were added Cs₂CO₃ (46.52 mg, 0.143 mmol, 3 equiv) and EPhos Pd G4 (8.74 mg, 0.010 mmol, 0.2 equiv) in portions at RT under N2 atmosphere. The resulting mixture was stirred for 16 hours at 50 degrees C. under N₂ atmosphere. The resulting mixture was filtered, then the filter cake was washed with EA (1×1 10 mL). The filtrate was concentrated under reduced pressure. The residue was purified by Prep-TLC (DCM and MeOH 25:1) to afford 3-[(3-chloro-2-methoxyphenyl)amino]-2-(6-methoxy-1,7-naphthyridin-4-yl)-1H,5H,6H,7H-pyrrolo[3,2-c]pyridin-4-one (1.8 mg, 8.11%) as a yellow solid.

LC-MS: (M+H)⁺ found 449.95.

¹H NMR (300 MHz, DMSO-d6) δ 11.80 (s, 1H), 9.08 (s, 1H), 8.82 (d, J=4.4 Hz, 1H), 7.74 (s, 1H), 7.63-7.56 (m, 1H), 7.25 (d, J=14.4 Hz, 2H), 6.51 (d, J=8.3 Hz, 1H), 6.38 (t, J=8.1 Hz, 1H), 6.05 (d, J=8.2 Hz, 1H), 3.90 (s, 3H), 3.82 (s, 3H), 3.49 (s, 2H), 2.92 (d, J=7.4 Hz, 2H).

### Example 55. (7R)-3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (23 mg) as a yellow solid and (7S)-3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (compound 774)

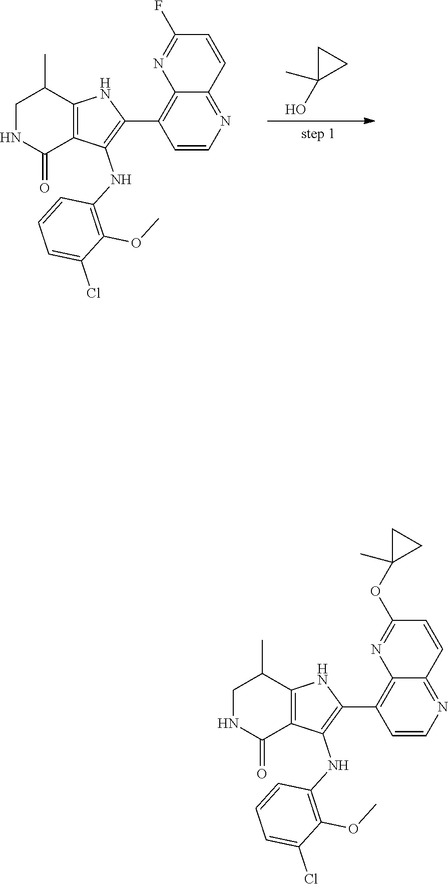

A solution of 3-[(3-chloro-2-methoxyphenyl)amino]-2-(6-fluoro-1,5-naphthyridin-4-yl)-7-methyl-1H,5H,6H,7H-pyrrolo[3,2-c]pyridin-4-one (564.02 mg, 1.248 mmol, 0.9 equiv) in DMF was treated with NaH (66.56 mg, 2.774 mmol, 2 equiv) for 30 min at 0° C. under nitrogen atmosphere followed by the addition of 3-[(3-chloro-2-methoxyphenyl)amino]-2-(6-fluoro-1,5-naphthyridin-4-yl)-7-methyl-1H,5H,6H,7H-pyrrolo[3,2-c]pyridin-4-one (564.02 mg, 1.248 mmol, 0.9 equiv) dropwise/in portions at RT for 2 h. The reaction was ok. The aqueous layer was extracted with EA (3×10 ml). The resulting mixture was concentrated under reduced pressure. The residue was purified by Prep-TLC (DCM/MeOH=10:1) to afford 3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (50 mg, 7.15%) as a light yellow solid.

LC-MS: M+H found: 504.

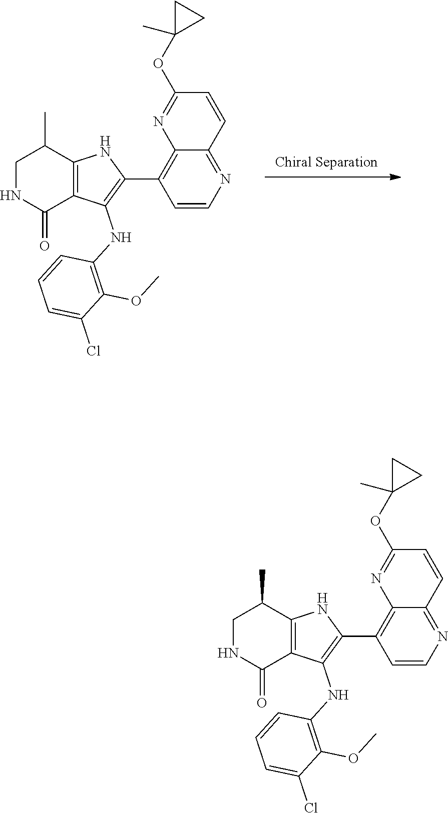 [SMILES: COc1c(Cl)cccc1Nc1c(-c2ccnc3ccc(OC4(C)CC4)nc23)[nH]c2c1C(=O)NCC2C ; COc1c(Cl)cccc1Nc1c(-c2ccnc3ccc(OC4(C)CC4)nc23)[nH]c2c1C(=O)NC[C@H]2C]

The crude product 3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (50.00 mg) was purified by Prep-chiral-HPLC with the following conditions (Column: CHIRALPAK IG-3, 4.6*50 mm, 3 um; Mobile Phase A: (Hex:DCM=3:1)(0.1% DEA): EtOH=50: 50; Flow rate: 1 mL/min; Gradient: 0% B to 0% B; Injection Volume: 5 ul mL) fractions containing the desired compound were evaporated to dryness to afford (7R)-3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (23 mg) as a yellow solid and (7S)-3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (23 mg) as a yellow solid.

LC-MS: M+H found: 504.

¹H NMR (400 MHz, DMSO-d6) δ 12.56 (s, 1H), 8.58 (d, J=4.9 Hz, 1H), 8.31 (d, J=9.1 Hz, 1H), 7.81 (s, 1H), 7.52 (d, J=4.9 Hz, 1H), 7.31 (d, J=9.1 Hz, 1H), 7.25 (d, J=3.5 Hz, 1H), 6.79-6.67 (m, 2H), 6.20 (dd, J=7.9, 1.8 Hz, 1H), 3.95 (s, 3H), 3.64 (dd, J=12.1, 4.7 Hz, 1H), 3.17 (ddt, J=15.9, 12.1, 4.1 Hz, 2H), 2.08 (s, 1H), 1.79 (s, 3H), 1.30 (d, J=6.7 Hz, 3H), 1.20 (dp, J=18.0, 6.3, 5.6 Hz, 2H), 0.96 (q, J=4.8 Hz, 2H).

### Example 56. (7R)-3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (compound 775)

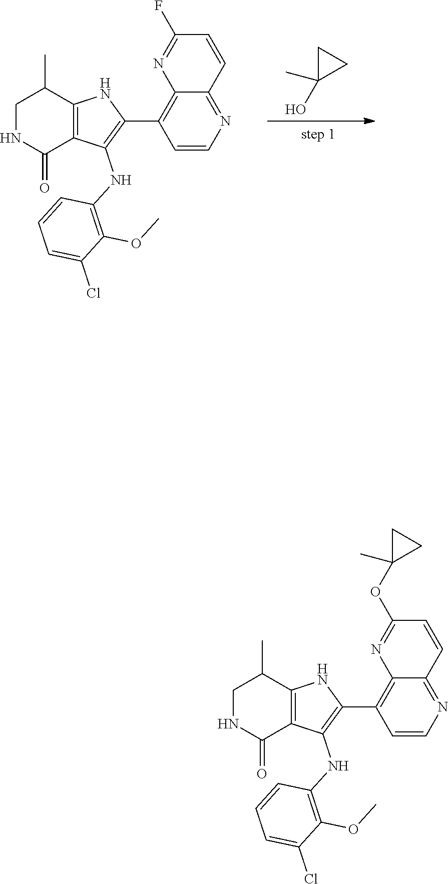

A solution of 3-[(3-chloro-2-methoxyphenyl)amino]-2-(6-fluoro-1,5-naphthyridin-4-yl)-7-methyl-1H,5H,6H,7H-pyrrolo[3,2-c]pyridin-4-one (564.02 mg, 1.248 mmol, 0.9 equiv) in DMF was treated with NaH (66.56 mg, 2.774 mmol, 2 equiv) for 30 min at 0° C. under nitrogen atmosphere followed by the addition of 3-[(3-chloro-2-methoxyphenyl)amino]-2-(6-fluoro-1,5-naphthyridin-4-yl)-7-methyl-1H,5H,6H,7H-pyrrolo[3,2-c]pyridin-4-one (564.02 mg, 1.248 mmol, 0.9 equiv) dropwise/in portions at RT for 2 h. The reaction was ok. The aqueous layer was extracted with EA (3×10 ml). The resulting mixture was concentrated under reduced pressure. The residue was purified by Prep-TLC (DCM/MeOH=10:1) to afford 3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (50 mg, 7.15%) as a light yellow solid.

LC-MS: M+H found: 504.

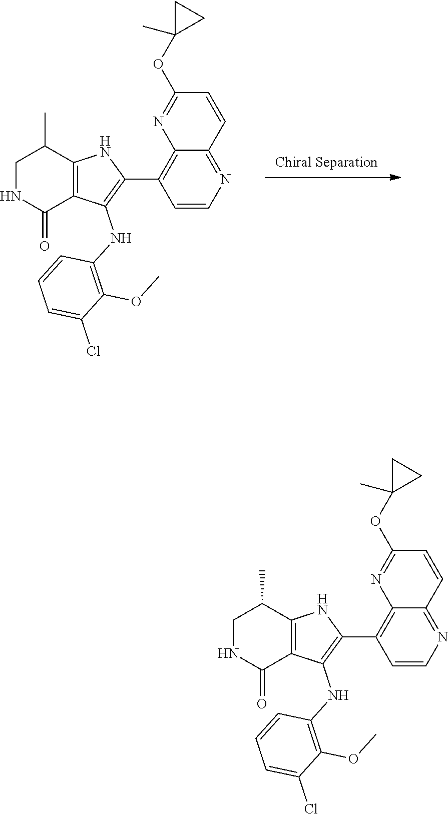 [SMILES: COc1c(Cl)cccc1Nc1c(-c2ccnc3ccc(OC4(C)CC4)nc23)[nH]c2c1C(=O)NCC2C ; COc1c(Cl)cccc1Nc1c(-c2ccnc3ccc(OC4(C)CC4)nc23)[nH]c2c1C(=O)NC[C@@H]2C]

The crude product 3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (50.00 mg) was purified by Prep-chiral-HPLC with the following conditions (Column: CHIRALPAK IG-3, 4.6*50 mm, 3 um; Mobile Phase A: (Hex:DCM=3:1)(0.1% DEA): EtOH=50: 50; Flow rate: 1 mL/min; Gradient: 0% B to 0% B; Injection Volume: 5 ul mL) fractions containing the desired compound were evaporated to dryness to afford (7R)-3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (23 mg) as a yellow solid and (7S)-3-[(3-chloro-2-methoxyphenyl)amino]-7-methyl-2-[6-(1-methylcyclopropoxy)-1,5-naphthyridin-4-yl]-1H,5H,6H,7H-pyrrolo[3,2-c]pyridin-4-one (23 mg) as a yellow solid.

LC-MS: M+H found: 504.

¹H NMR (400 MHz, DMSO-d6) δ 12.56 (s, 1H), 8.58 (d, J=4.9 Hz, 1H), 8.31 (d, J=9.1 Hz, 1H), 7.81 (s, 1H), 7.52 (d, J=4.9 Hz, 1H), 7.31 (d, J=9.1 Hz, 1H), 7.25 (d, J=3.5 Hz, 1H), 6.79-6.67 (m, 2H), 6.20 (dd, J=7.9, 1.8 Hz, 1H), 3.95 (s, 3H), 3.64 (dd, J=12.0, 4.8 Hz, 1H), 3.17 (ddt, J=15.9, 12.2, 4.1 Hz, 2H), 2.08 (s, 1H), 1.79 (s, 3H), 1.30 (d, J=6.7 Hz, 3H), 1.20 (tt, J=10.5, 5.6 Hz, 2H), 0.96 (q, J=4.9 Hz, 2H).

### Example 57. (7R)-3-[(3-chloro-2-methoxyphenyl)amino]-2-(3-fluoropyridin-4-yl)-7-(2-methoxyethyl)-7-methyl-1H,5H,6H-pyrrolo[3,2-c]pyridin-4-one (compound 780)

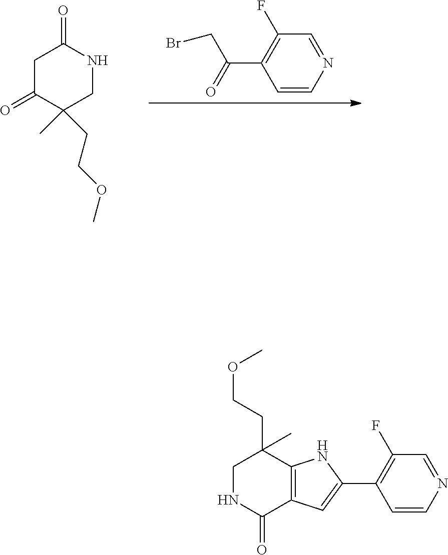 [SMILES: COCCC1(C)CNC(=O)CC1=O ; O=C(CBr)c1ccncc1F ; COCCC1(C)CNC(=O)c2cc(-c3ccncc3F)[nH]c21]

To a stirred solution of 5-(2-methoxyethyl)-5-methylpiperidine-2,4-dione (705 mg, 3.806 mmol, 1.00 equiv) and 2-bromo-1-(3-fluoropyridin-4-yl)ethanone (829.85 mg, 3.806 mmol, 1 equiv) in EtOH (10 mL, 172.135 mmol, 45.22 equiv) was added NH₄OAc (1466.97 mg, 19.030 mmol, 5 equiv) in portions at RT. The resulting mixture was stirred for 10 hours at 50 degrees C. under N2 atmosphere. The mixture was allowed to cool down to RT. Desired product could be detected by LCMS. The crude resulting mixture was used in the next step directly without further purification.

LC-MS: M+H found: 304.25.

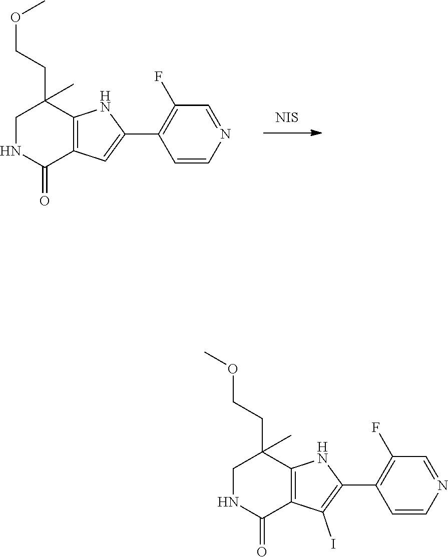

To a stirred solution of bis(2-(3-fluoropyridin-4-yl)-7-(2-methoxyethyl)-7-methyl-1H,5H,6H-pyrrolo[3,2-c]pyridin-4-one) (1000 mg, 1.648 mmol, 1.00 equiv) in DMF (10 mL, 129.218 mmol, 78.39 equiv) was added NIS (556.27 mg, 2.472 mmol, 1.5 equiv) in portions at RT. The resulting mixture was stirred for 1 hour at RT. The resulting mixture was extracted with EA (3×10 mL). The combined organic layer was concentrated under reduced pressure. The residue was purified by Prep-TLC (DCM:MeOH 15:1) to afford bis(2-(3-fluoropyridin-4-yl)-3-iodo-7-(2-methoxyethyl)-7-methyl-1H,5H,6H-pyrrolo[3,2-c]pyridin-4-one) (960 mg, 67.84%) as a light yellow solid.

LC-MS: M+H found: 430.15.

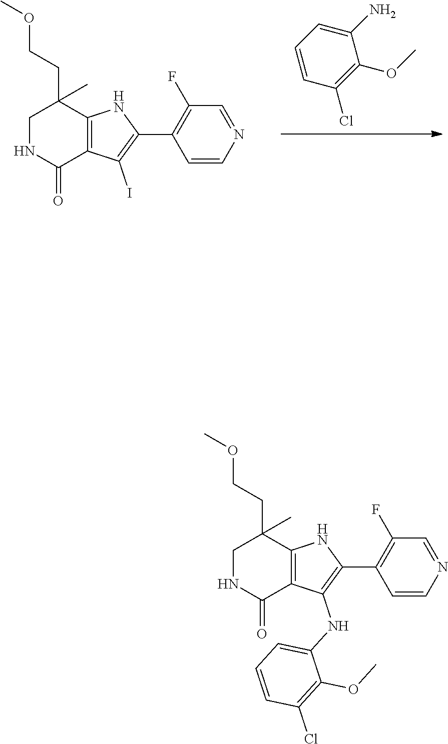

To a stirred mixture of 2-(3-fluoropyridin-4-yl)-3-iodo-7-(2-methoxyethyl)-7-methyl-1H,5H,6H-pyrrolo[3,2-c]pyridin-4-one (480 mg, 1.118 mmol, 1.00 equiv) and EPhos Pd G4 (205.44 mg, 0.224 mmol, 0.2 equiv) and Cs₂CO₃ (1093.06 mg, 3.354 mmol, 3 equiv) in DMF (5 mL) was added 3-chloro-2-methoxyaniline (528.72 mg, 3.354 mmol, 3 equiv) in portions at RT under N2 atmosphere. The resulting mixture was stirred for 2 hours at 50 degrees C. under N2 atmosphere. The resulting mixture was filtered, then the filter cake was washed with EA (1×1 10 mL). The filtrate was concentrated under reduced pressure. The residue was purified by Prep-TLC (DCM and MeOH 20:1) to afford 3-[(3-chloro-2-methoxyphenyl)amino]-2-(3-fluoropyridin-4-yl)-7-(2-methoxyethyl)-7-methyl-1H,5H,6H-pyrrolo[3,2-c]pyridin-4-one (275 mg, 53.59%) as a light yellow solid.

LC-MS: M+H found: 459.15.

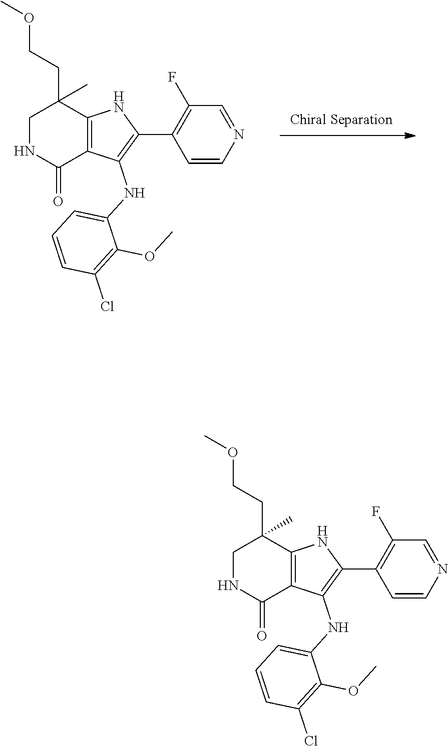 [SMILES: COCCC1(C)CNC(=O)c2c1[nH]c(-c1ccncc1F)c2Nc1cccc(Cl)c1OC ; COCC[C@]1(C)CNC(=O)c2c1[nH]c(-c1ccncc1F)c2Nc1cccc(Cl)c1OC]

The crude product 3-[(3-chloro-2-methoxyphenyl)amino]-2-(3-fluoropyridin-4-yl)-7-(2-methoxyethyl)-7-methyl-1H,5H,6H-pyrrolo[3,2-c]pyridin-4-one (300.00 mg, 0.654 mmol, 1.00 equiv) was purified by Prep-Chiral-HPLC to afford (7R)-3-[(3-chloro-2-methoxyphenyl)amino]-2-(3-fluoropyridin-4-yl)-7-(2-methoxyethyl)-7-methyl-1H,5H,6H-pyrrolo[3,2-c]pyridin-4-one (117 mg, 38.81%) as a off-white solid.

LC-MS: (M+H)⁺ found 459.0.

¹H NMR (400 MHz, DMSO-d6) δ 11.37 (s, 1H), 8.50 (d, J=2.8 Hz, 1H), 8.32 (dd, J=5.0, 1.1 Hz, 1H), 7.65 (s, 1H), 7.45 (dd, J=6.8, 5.1 Hz, 1H), 7.28 (d, J=2.7 Hz, 1H), 6.70-6.59 (m, 2H), 6.11 (dd, J=7.6, 2.1 Hz, 1H), 3.85 (s, 3H), 3.43 (td, J=6.7, 2.8 Hz, 2H), 3.29-3.24 (m, 4H), 3.17 (dd, J=12.5, 2.4 Hz, 1H), 1.99 (dt, J=14.0, 7.0 Hz, 1H), 1.84 (dt, J=13.6, 6.5 Hz, 1H), 1.33 (s, 3H).

### Example 58. (7S)-3-[(3-chloro-2-methoxyphenyl)amino]-2-(3-fluoropyridin-4-yl)-7-(2-methoxyethyl)-7-methyl-1H,5H,6H-pyrrolo[3,2-c]pyridin-4-one (compound 779)

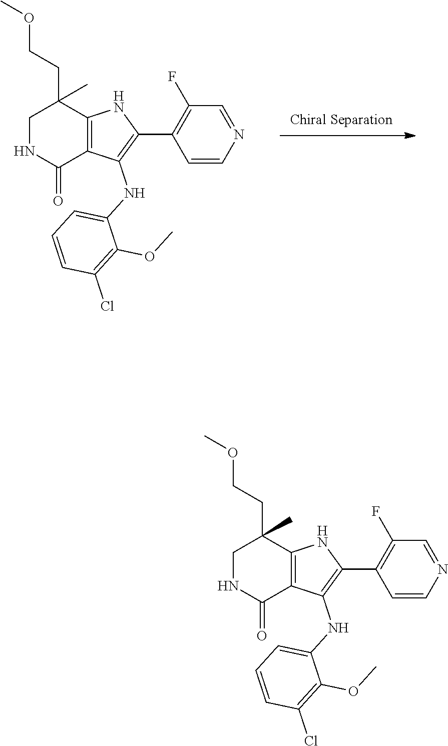 [SMILES: COCCC1(C)CNC(=O)c2c1[nH]c(-c1ccncc1F)c2Nc1cccc(Cl)c1OC ; COCC[C@@]1(C)CNC(=O)c2c1[nH]c(-c1ccncc1F)c2Nc1cccc(Cl)c1OC]

The crude product 3-[(3-chloro-2-methoxyphenyl)amino]-2-(3-fluoropyridin-4-yl)-7-(2-methoxyethyl)-7-methyl-1H,5H,6H-pyrrolo[3,2-c]pyridin-4-one (300.00 mg, 0.654 mmol, 1.00 equiv) was purified by Prep-Chiral-HPLC to afford (7S)-3-[(3-chloro-2-methoxyphenyl)amino]-2-(3-fluoropyridin-4-yl)-7-(2-methoxyethyl)-7-methyl-1H,5H,6H-pyrrolo[3,2-c]pyridin-4-one (111.5 mg, 36.98%) as a off-white solid.

LC-MS: (M+H)⁺ found: 459. 0.

¹H NMR (400 MHz, DMSO-d6) δ 11.37 (s, 1H), 8.50 (d, J=2.7 Hz, 1H), 8.32 (dd, J=5.0, 1.1 Hz, 1H), 7.66 (s, 1H), 7.45 (dd, J=6.8, 5.1 Hz, 1H), 7.28 (t, J=2.7 Hz, 1H), 6.70-6.59 (m, 2H), 6.11 (dd, J=7.6, 2.1 Hz, 1H), 3.85 (s, 3H), 3.43 (dt, J=6.8, 3.3 Hz, 2H), 3.31-3.24 (m, 4H), 3.17 (dd, J=12.5, 2.4 Hz, 1H), 1.99 (dt, J=13.9, 6.9 Hz, 1H), 1.84 (dt, J=13.6, 6.5 Hz, 1H), 1.33 (s, 3H).

### Example 59. (7R)-3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one (13.9 mg) as a white solid and (7S)-3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one (compound 797)

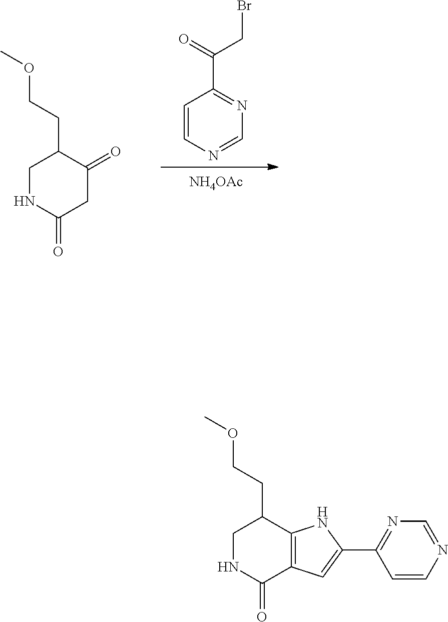

To a mixture of 5-(2-methoxyethyl)piperidine-2,4-dione (1.5 g, 8.762 mmol, 1 equiv) in EtOH (2 mL) was added ammonium acetate (1350.79 mg, 17.524 mmol, 2 equiv) and 2-bromo-1-(pyrimidin-4-yl)ethanone (2113.61 mg, 10.514 mmol, 1.2 equiv), the reaction mixture was stirred at 50° C. for 16 h, LCMS was OK. The reaction mixture was concentrated under vacuum. The residue was purified by flash chromatography (9% MeoH in DCM) to give 7-(2-methoxyethyl)-2-(pyrimidin-4-yl)-1H,5H,6H,7H-pyrrolo[3,2-c]pyridin-4-one (750 mg, 31.43%) as a red solid.

LC-MS: M+H found: 273.

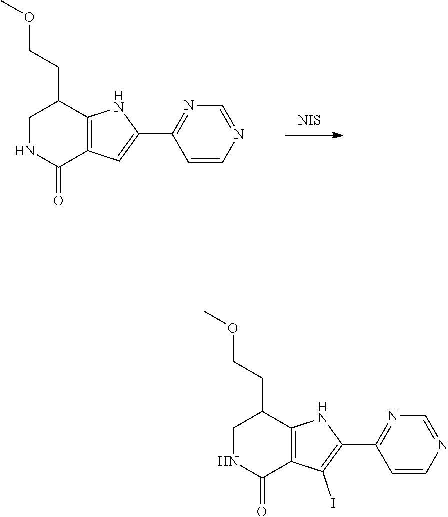

To a mixture of 7-(2-methoxyethyl)-2-(pyrimidin-4-yl)-1H,5H,6H,7H-pyrrolo[3,2-c]pyridin-4-one (750 mg, 2.754 mmol, 1 equiv) in DMF (7 mL) was added iodo(sulfanyl)amine (575.02 mg, 3.305 mmol, 1.2 equiv), the reaction mixture was stirred at 50° C. for 2 h. LCMS was ok. The reaction mixture was added to the saturated and extracted with DCM. The organic phase was concentrated under vacuum. The residue was purified by flash chromatography (9% MeOH in DCM) to give 3-iodo-7-(2-methoxyethyl)-2-(pyrimidin-4-yl)-1H,5H,6H,7H-pyrrolo[3,2-c]pyridin-4-one (700 mg, 63.83%) as a red solid.

LC-MS: M+H found: 399.

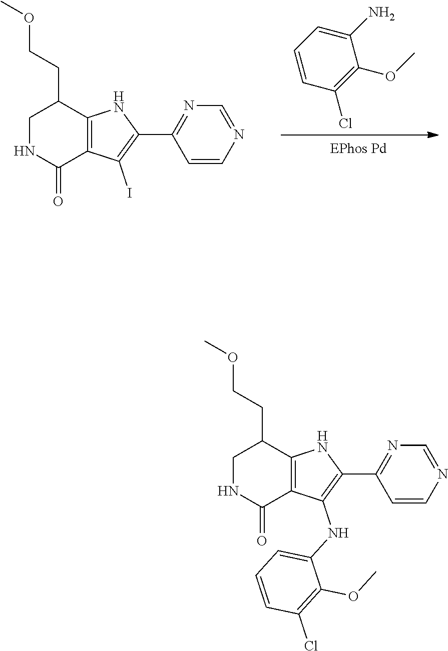

Into a 20-mL sealed tube was placed 3-iodo-7-(2-methoxyethyl)-2-(pyrimidin-4-yl)-1H,5H,6H,7H-pyrrolo[3,2-c]pyridin-4-one (750 mg, 1.883 mmol, 1 equiv) in DMF (7 mL), 3-chloro-2-methoxyaniline (890.50 mg, 5.649 mmol, 3 equiv), EPhos Pd G4 (692.02 mg, 0.753 mmol, 0.4 equiv) and Cs₂CO₃ (1227.33 mg, 3.766 mmol, 2 equiv). The resulting solution was stirred at 50° C. for 2 h. The reaction mixture was added to the ice water and extracted with EA. The organic phase was concentrated under vacuum. the residue was purified by Prep-Flash-HPLC with following conditions (9% MeOH in DCM). This resulted in 3-[(3-chloro-2-methoxyphenyl)amino]-7-(2-methoxyethyl)-2-(pyrimidin-4-yl)-1H,5H,6H,7H-pyrrolo[3,2-c]pyridin-4-one (160 mg, 19.85%) as a light yellow solid.

LC-MS: M+H found: 428

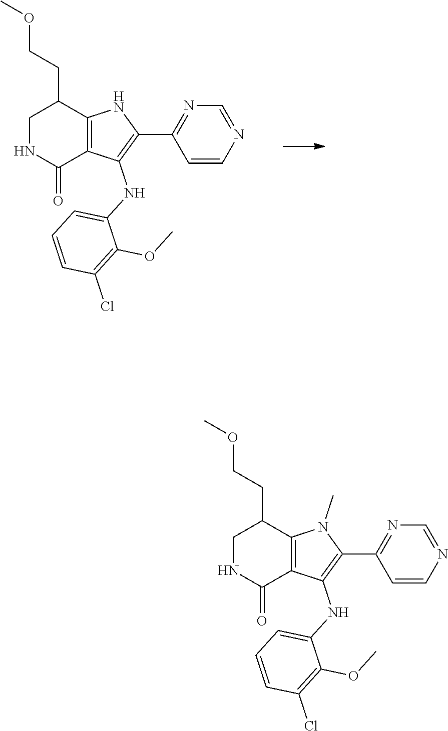 [SMILES: COCCC1CNC(=O)c2c1[nH]c(-c1ccncn1)c2Nc1cccc(Cl)c1OC ; COCCC1CNC(=O)c2c(Nc3cccc(Cl)c3OC)c(-c3ccncn3)n(C)c21]

The reaction mixture was added to the saturated and extracted with DCM. The organic phase was concentrated under vacuum. The residue was purified by flash chromatography (9% MeOH in DCM) to give 3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one (50 mg, 30.26%) as a light yellow solid.

LC-MS: M+H found 428.

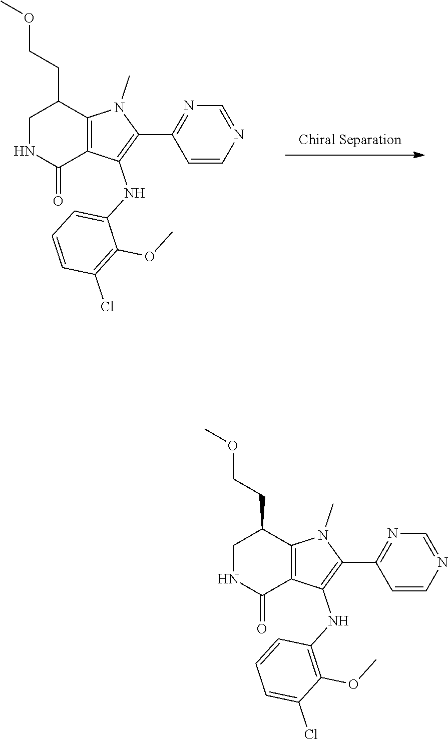 [SMILES: COCCC1CNC(=O)c2c(Nc3cccc(Cl)c3OC)c(-c3ccncn3)n(C)c21 ; COCC[C@@H]1CNC(=O)c2c(Nc3cccc(Cl)c3OC)c(-c3ccncn3)n(C)c21]

The crude product 3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one) was purified by Prep-chiral-HPLC with the following conditions (Column: CHIRALPAK IG-3, 4.6*50 mm 3 um; Mobile Phase A: (Hex:DCM=3:1)(0.1% DEA):IPA=70:30; Flow rate: 1 mL/min; Gradient: 0% B to 0% B; Injection Volume: 5 ul mL) fractions containing the desired compound were evaporated to dryness to afford (7R)-3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one (13.9 mg) as a white solid and (7S)-3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one (16.9 mg) as a white solid.

LC-MS: M+H found: 442.

¹H NMR (300 MHz, DMSO-d6) δ 9.14 (s, 1H), 8.56 (d, J=5.4 Hz, 1H), 7.69 (s, 1H), 7.42 (d, J=5.5 Hz, 1H), 7.17 (s, 1H), 6.69 (d, J=5.2 Hz, 2H), 6.13 (dd, J=6.1, 3.2 Hz, 1H), 3.88 (d, J=6.2 Hz, 6H), 3.55-3.35 (m, 4H), 3.28 (s, 3H), 3.18 (s, 1H), 1.88-1.71 (m, 2H).

### Example 60. (7S)-3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one Compound 796)

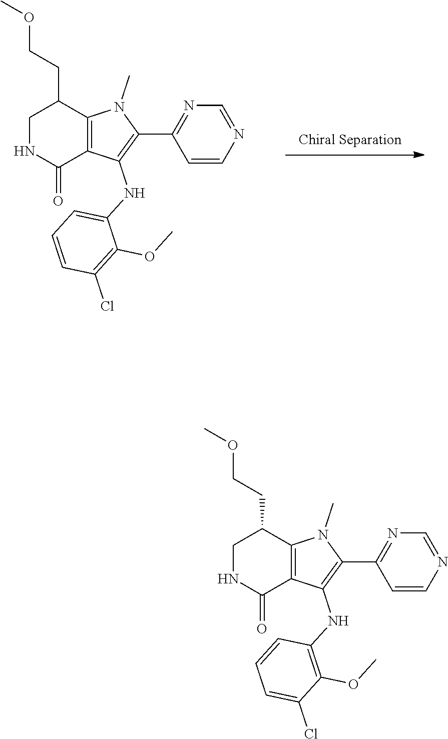 [SMILES: COCCC1CNC(=O)c2c(Nc3cccc(Cl)c3OC)c(-c3ccncn3)n(C)c21 ; COCC[C@H]1CNC(=O)c2c(Nc3cccc(Cl)c3OC)c(-c3ccncn3)n(C)c21]

The crude product 3-[(3-chloro-2-methoxyphenyl)amino]-7-(2-methoxyethyl)-I-methyl-2-(pyrimidin-4-yl)-5H,6H,7H-pyrrolo[3,2-c]pyridin-4-one) was purified by Prep-chiral-HPLC with the following conditions (Column: CHIRALPAK IG-3, 4.6*50 mm 3 um; Mobile Phase A: (Hex:DCM=3:1)(0.1% DEA): IPA=70: 30; Flow rate: 1 mL/min; Gradient: 0% B to 0% B; Injection Volume: 5 ul mL) fractions containing the desired compound were evaporated to dryness to afford (7R)-3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one (13.9 mg) as a white solid and (7S)-3-[(3-chloro-2-methoxyphenyl)amino]-7-(2-methoxyethyl)-1-methyl-2-(pyrimidin-4-yl)-5H,6H,7H-pyrrolo[3,2-c]pyridin-4-one (16.9 mg) as a white solid.

LC-MS: M+H found: 442.

¹H NMR (300 MHz, DMSO-d6) δ 9.14 (s, 1H), 8.56 (d, J=5.6 Hz, 1H), 7.69 (s, 1H), 7.42 (d, J=5.6 Hz, 1H), 7.18 (s, 1H), 6.72-6.64 (m, 2H), 6.13 (dd, J=6.7, 3.3 Hz, 1H), 3.88 (d, J=6.2 Hz, 6H), 3.54-3.37 (m, 4H), 3.28 (s, 3H), 3.18 (s, 1H), 1.83-1.74 (m, 2H).

### Example 61. (7R)-3-[(3-chloro-2-methoxyphenyl)amino]-7-(2,2-difluoroethyl)-2-(3-fluoropyridin-4-yl)-1H,5H,6H,7H-pyrrolo[3,2-c]pyridin-4-one (compound 820)

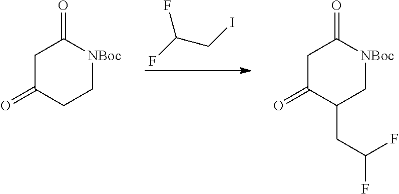 [SMILES: CC(C)(C)OC(=O)N1CCC(=O)CC1=O ; FC(F)CI ; CC(C)(C)OC(=O)N1CC(CC(F)F)C(=O)CC1=O]

To a stirred mixture of pyrimidin-5-ol (2.00 g, 20.814 mmol, 1.00 equiv) and 2-bromoethyl methyl ether (3.47 g, 0.025 mmol, 1.2 equiv) in DMF (15.00 mL, 193.826 mmol, 9.31 equiv) was added K2CO3 (5.75 g, 0.042 mmol, 2.0 equiv) at rt under N2 atmosphere. Then, the solution was stirred at 60 degrees C. for 3 h. TLC: DCM:MeOH=15:1. The resulting mixture was diluted with water (50 mL) and washed with 6×60 mL of EA. The filtrate was concentrated under reduced pressure. The residue was purified by reverse flash chromatography with the following conditions: column, C18 silica gel; mobile phase, MeOH in DCM, 0% to 5% gradient in 10 min; detector, UV 254 nm to afford 5-(2-methoxyethoxy) pyrimidine (0.848 g) as a yellow oil.

LC-MS: M+H found: 221.95.

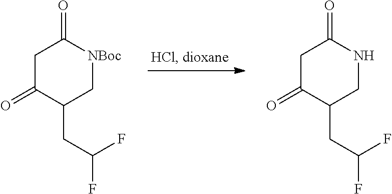 [SMILES: CC(C)(C)OC(=O)N1CC(CC(F)F)C(=O)CC1=O ; O=C1CC(=O)C(CC(F)F)CN1]

To a stirred solution of tert-butyl 5-(2,2-difluoroethyl)-2,4-dioxopiperidine-1-carboxylate (1.05 g, 3.787 mmol, 1.00 equiv) in DCM (7.5 mL, 117.975 mmol, 31.15 equiv) was added HCl (gas) in 1,4-dioxane (2.5 mL, 82.280 mmol, 21.73 equiv) dropwise at RT.

Desired product could be detected by LCMS. The crude resulting mixture was used in the next step directly without further purification.

LC-MS: M+H found: 178.1.

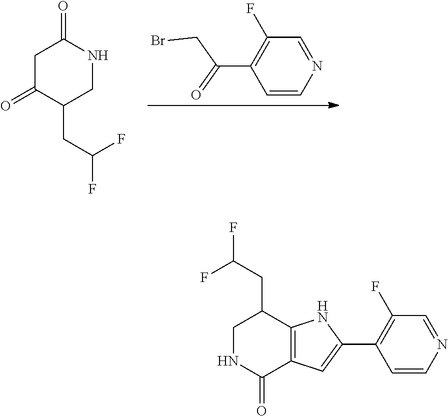 [SMILES: O=C1CC(=O)C(CC(F)F)CN1 ; O=C(CBr)c1ccncc1F ; O=C1NCC(CC(F)F)c2[nH]c(-c3ccncc3F)cc21]

To a stirred solution of 5-(2,2-difluoroethyl)piperidine-2,4-dione (800 mg, 4.516 mmol, 1.00 equiv) and NH₄OAc (1740.49 mg, 22.580 mmol, 5 equiv) in EtOH (8 mL, 137.708 mmol, 30.49 equiv) was added 2-bromo-1-(3-fluoropyridin-4-yl)ethanone (1181.50 mg, 5.419 mmol, 1.2 equiv) dropwise at RT. The resulting mixture was stirred for 16 hours at 50 degrees C. under N2 atmosphere. The aqueous layer was extracted with EA and H2O (3×150 mL). The combined organic phase was concentrated to afford 7-(2,2-difluoroethyl)-2-(3-fluoropyridin-4-yl)-1H,5H,6H,7H-pyrrolo[3,2-c]pyridin-4-one (825 mg, 61.87%) as a light yellow solid.

LC-MS: M+H found: 296.2.

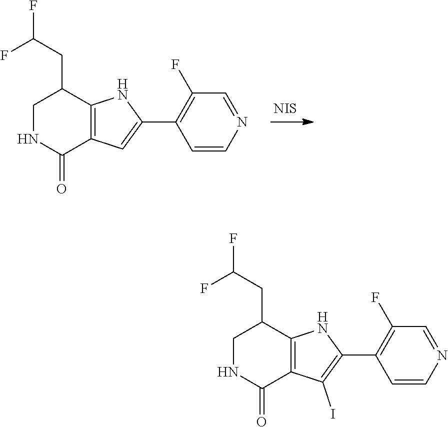

To a stirred solution of 7-(2,2-difluoroethyl)-2-(3-fluoropyridin-4-yl)-1H,5H,6H,7H-pyrrolo[3,2-c]pyridin-4-one (800 mg, 2.709 mmol, 1.00 equiv) in DMF (8 mL, 103.374 mmol, 38.15 equiv) was added NIS (914.37 mg, 4.064 mmol, 1.5 equiv) in portions at RT. The resulting mixture was stirred for 1 hour at RT. The resulting mixture was extracted with EA (3×10 mL). The combined organic layer was concentrated under reduced pressure. The residue was purified by Prep-TLC (DCM and MeOH 25:1) to afford 7-(2,2-difluoroethyl)-2-(3-fluoropyridin-4-yl)-3-iodo-1H,5H,6H,7H-pyrrolo[3,2-c]pyridin-4-one (358 mg, 31.37%) as a light yellow solid.

LC-MS: M+H found: 243.05.

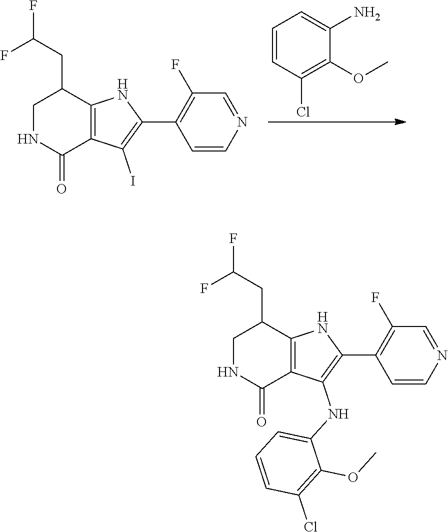

To a stirred mixture of 2-(3-fluoropyridin-4-yl)-3-iodo-7-(2-methoxyethyl)-1H,5H,6H,7H-pyrrolo[3,2-c]pyridin-4-one (500 mg, 1.204 mmol, 1.00 equiv) and EPhos (128.80 mg, 0.241 mmol, 0.2 equiv) and EPhos Pd G4 (221.23 mg, 0.241 mmol, 0.2 equiv) and Cs2CO3 (1177.07 mg, 3.612 mmol, 3 equiv) in DMF (5 mL) was added 3-fluoro-2-methoxyaniline (509.91 mg, 3.612 mmol, 3 equiv) in portions at RT under N2 atmosphere. The resulting mixture was stirred for 2 hours at 50 degrees C. under N2 atmosphere. The resulting mixture was filtered, then the filter cake was washed with EA (1×1 10 mL). The filtrate was concentrated under reduced pressure. The residue was purified by Prep-TLC (DCM and MeOH 20:1) to afford 3-[(3-fluoro-2-methoxyphenyl)amino]-2-(3-fluoropyridin-4-yl)-7-(2-methoxyethyl)-1H,5H,6H,7H-pyrrolo[3,2-c]pyridin-4-one (410 mg, 79.47%) as a light yellow solid.

LC-MS: M+H found: 450.95.

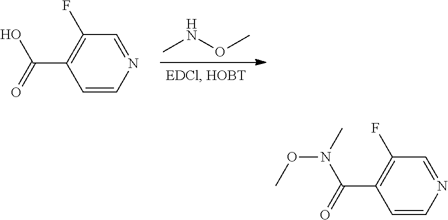

To a stirred mixture of 3-fluoropyridine-4-carboxylic acid (6 g, 42.523 mmol, 1.00 equiv) and N,O-dimethylhydroxylamine (3.90 g, 63.785 mmol, 1.5 equiv) and EDCI (8.97 g, 46.775 mmol, 1.1 equiv) and HOBT (6.32 g, 46.775 mmol, 1.1 equiv) in DCM (150 mL, 2359.507 mmol, 55.49 equiv) was added TEA (17.21 g, 170.092 mmol, 4 equiv) dropwise at RT. The resulting mixture was stirred for 16 hours at RT under N2 atmosphere. The aqueous layer was extracted with DCM and H2O (1×1 500 mL). The crude product was purified by Prep-HPLC to afford 3-fluoro-N-methoxy-N-methylpyridine-4-carboxamide (4.37 g, 55.80%) as a light yellow oil.

LC-MS: M+H found: 185.1.

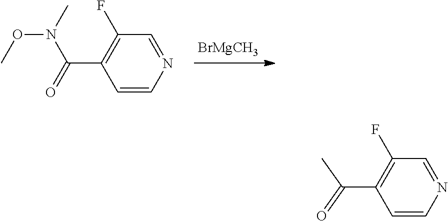

To a stirred solution of 3-fluoro-N-methoxy-N-methylpyridine-4-carboxamide; bromo(methyl)magnesium (3.87 g, 12.755 mmol, 1.00 equiv) in THF (40 mL) was added bromo(methyl)magnesium (2.28 g, 19.133 mmol, 1.5 equiv) dropwise at 0° C. under N2 atmosphere. The resulting mixture was stirred for 0.5 hour at rt under N2 atmosphere. The reaction was quenched with NH4Cl at 0° C. The aqueous layer was extracted with EA and H2O (1×1 300 mL). To afford 1-(3-fluoropyridin-4-yl)ethanone (2.5 g, 91.57%) as a light yellow oil.

LC-MS: M+H found: 140.05.

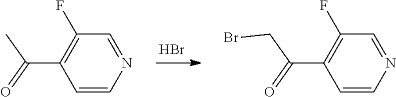

To a stirred solution of 1-(3-fluoropyridin-4-yl)ethanone (3 g, 21.563 mmol, 1.00 equiv) and hydrogen bromide (3 mL, 37.077 mmol, 1.72 equiv) in HAc (11 mL, 191.967 mmol, 8.90 equiv) was added Br2 (1.13 mL, 22.054 mmol, 1.02 equiv) dropwise at RT. The resulting mixture was stirred for 2.5 hours at 60° C. under N2 atmosphere. Desired product could be detected by LCMS. The mixture was allowed to cool down to RT. The product was precipitated by the addition of EA. The precipitated solids were collected by filtration to afford 2-bromo-1-(3-fluoropyridin-4-yl)ethanone (4.4 g, 93.59%) as a light yellow solid.

LC-MS: M+H found: 450.95.

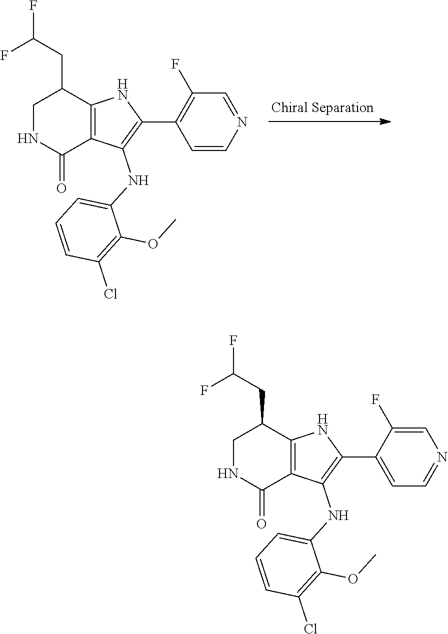 [SMILES: COc1c(Cl)cccc1Nc1c(-c2ccncc2F)[nH]c2c1C(=O)NCC2CC(F)F ; COc1c(Cl)cccc1Nc1c(-c2ccncc2F)[nH]c2c1C(=O)NC[C@H]2CC(F)F]

The crude product 3-[(3-chloro-2-methoxyphenyl)amino]-7-(2,2-difluoroethyl)-2-(3-fluoropyridin-4-yl)-1H,5H,6H,7H-pyrrolo[3,2-c]pyridin-4-one (100 mg, 0.222 mmol, 1.00 equiv) was purified by Prep-Chiral-HPLC with the following conditions (Column; CHIRALPAK IA-3, 4.6*50 mm 3 um; Mobile Phase A: MtBE (0.1% DEA):EtOH=90:10; Flow rate: 1 mL/min; Gradient: 0% B to 0% B; Injection Volume: 5 ul mL) to afford (7R)-3-[(3-chloro-2-methoxyphenyl)amino]-7-(2,2-difluoroethyl)-2-(3-fluoropyridin-4-yl)-1H,5H,6H,7H-pyrrolo[3,2-c]pyridin-4-one (33.4 mg, 33.30%) as a light yellow solid.

LC-MS: (M+H)⁺ found: 450.95.

¹H NMR (300 MHz, DMSO-d6) δ 11.63 (s, 1H), 8.52 (d, J=2.9 Hz, 1H), 8.32 (dd, J=5.1, 1.2 Hz, 1H), 7.60 (s, 1H), 7.47 (dd, J=6.8, 5.1 Hz, 1H), 7.27 (s, 1H), 6.73-6.59 (m, 2H), 6.29 (t, J=4.5 Hz, 1H), 6.12 (td, J=8.0, 7.6, 3.5 Hz, 1H), 3.86 (s, 3H), 3.58 (d, J=9.6 Hz, 1H), 2.39 (q, J=17.8, 17.0 Hz, 2H).

### Example 62. (7S)-3-[(3-chloro-2-methoxyphenyl)amino]-7-(2,2-difluoroethyl)-2-(3-fluoropyridin-4-yl)-1H,5H,6H,7H-pyrrolo[3,2-c]pyridin-4-one (compound 819)

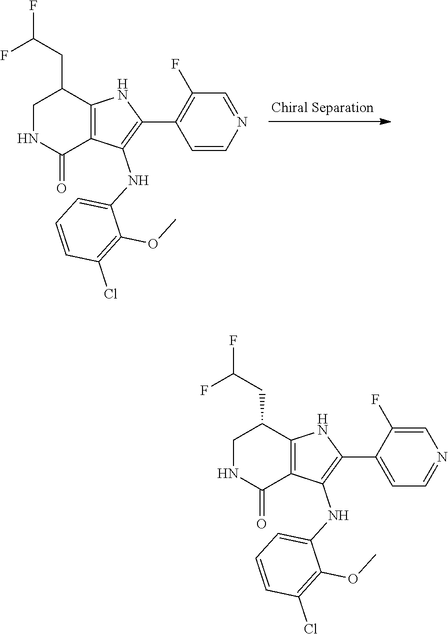 [SMILES: COc1c(Cl)cccc1Nc1c(-c2ccncc2F)[nH]c2c1C(=O)NCC2CC(F)F ; COc1c(Cl)cccc1Nc1c(-c2ccncc2F)[nH]c2c1C(=O)NC[C@@H]2CC(F)F]

The crude product 3-[(3-chloro-2-methoxyphenyl)amino]-7-(2,2-difluoroethyl)-2-(3-fluoropyridin-4-yl)-1H,5H,6H,7H-pyrrolo[3,2-c]pyridin-4-one (100 mg, 0.222 mmol, 1.00 equiv) was purified by Prep-Chiral-HPLC with the following conditions (Column: CHIRALPAK IA-3, 4.6*50 mm 3 um; Mobile Phase A: MtBE (0.1% DEA): EtOH=90: 10; Flow rate: 1 mL/min; Gradient: 0% B to 0% B; Injection Volume: 5 ul mL) to afford (7S)-3-[(3-chloro-2-methoxyphenyl)amino]-7-(2,2-difluoroethyl)-2-(3-fluoropyridin-4-yl)-1H,5H,6H,7H-pyrrolo[3,2-c]pyridin-4-one (35.4 mg, 35.29%) as a light yellow solid.

LC-MS: (M+H)⁺ found 450.95.

¹H NMR (300 MHz, DMSO-d6) δ 11.63 (s, 1H), 8.52 (d, J=2.9 Hz, 1H), 8.32 (d, J=5.0 Hz, 1H), 7.60 (s, 1H), 7.47 (dd, J=6.8, 5.1 Hz, 1H), 7.27 (s, 1H), 6.72-6.59 (m, 2H), 6.50-6.06 (m, 2H), 3.86 (s, 3H), 3.59 (d, J=9.4 Hz, 1H), 2.37-2.03 (m, 2H).

### Example 63. (7R)-3-{[2-(difluoromethoxy)-3-fluorophenyl]amino}-2-(3-fluoropyridin-4-yl)-7-(2-methoxyethyl)-1H,5H,6H,7H-pyrrolo[3,2-c]pyridin-4-one (compound 470)

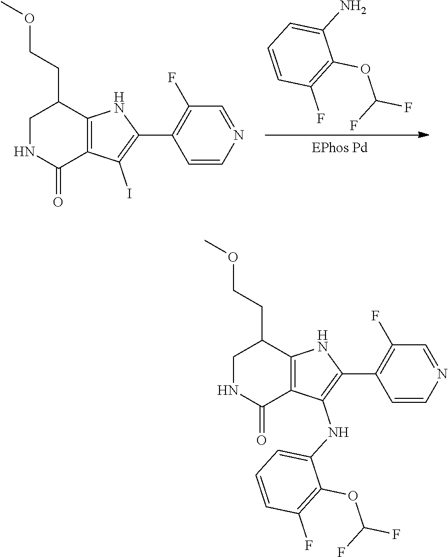 [SMILES: COCCC1CNC(=O)c2c1[nH]c(-c1ccncc1F)c2I ; Nc1cccc(F)c1OC(F)F ; COCCC1CNC(=O)c2c1[nH]c(-c1ccncc1F)c2Nc1cccc(F)c1OC(F)F]

To a stirred mixture of 2-(3-fluoropyridin-4-yl)-3-iodo-7-(2-methoxyethyl)-1H,5H,6H,7H-pyrrolo[3,2-c]pyridin-4-one (300 mg, 0.723 mmol, 1.00 equiv) and EPhos (77.28 mg, 0.145 mmol, 0.2 equiv) and EPhos Pd G4 (132.74 mg, 0.145 mmol, 0.2 equiv) and Cs₂CO₃ (706.24 mg, 2.169 mmol, 3 equiv) in DMF (3 mL) was added 2-(difluoromethoxy)-3-fluoroaniline (383.94 mg, 2.169 mmol, 3 equiv) in portions at RT under N2 atmosphere. The resulting mixture was stirred for 2 hours at 50 degrees C. under N2 atmosphere. The resulting mixture was filtered, then the filter cake was washed with EA (1×1 10 mL). The filtrate was concentrated under reduced pressure. The residue was purified by Prep-TLC (DCM and MeOH 20:1) to afford 3-{[2-(difluoromethoxy)-3-fluorophenyl]amino}-2-(3-fluoropyridin-4-yl)-7-(2-methoxyethyl)-1H,5H,6H,7H-pyrrolo[3,2-c]pyridin-4-one (263 mg, 78.38%) as a light yellow solid.

LC-MS: M+H found: 464.95.

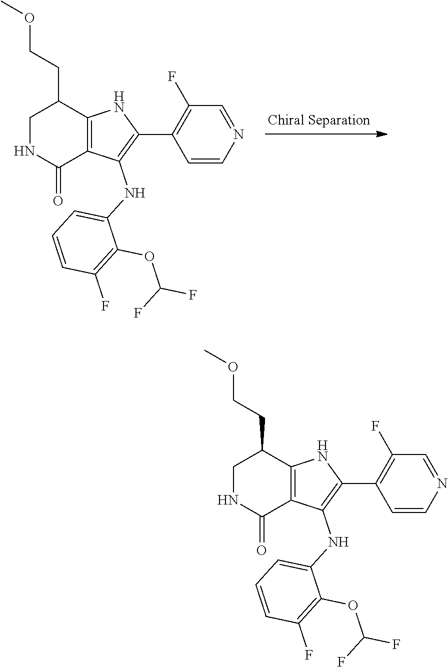 [SMILES: COCCC1CNC(=O)c2c1[nH]c(-c1ccncc1F)c2Nc1cccc(F)c1OC(F)F ; COCC[C@@H]1CNC(=O)c2c1[nH]c(-c1ccncc1F)c2Nc1cccc(F)c1OC(F)F]

The crude product 3-{[2-(difluoromethoxy)-3-fluorophenyl]amino}-2-(3-fluoropyridin-4-yl)-7-(2-methoxyethyl)-1H,5H,6H,7H-pyrrolo[3,2-c]pyridin-4-one (100 mg, 0.215 mmol, 1.00 equiv) was purified by Prep-Chiral-HPLC with the following conditions (Column: CHIRAL ART Amylose-SA, 2*25 cm, 5 μm; Mobile Phase A: Hex (0.5% 2M NH3-MeOH)-HPLC, Mobile Phase B: EtOH-HPLC; Flow rate: 20 mL/min; Gradient: 25% B to 25% B in 11 min; Wave Length: 220/254 nm; RT1 (min): 7.57; RT2 (min): 9.68; Sample Solvent: EtOH-HPLC; Injection Volume: 0.8 mL; Number Of Runs: 5) to afford (7R)-3-{[2-(difluoromethoxy)-3-fluorophenyl]amino}-2-(3-fluoropyridin-4-yl)-7-(2-methoxyethyl)-1H,5H,6H,7H-pyrrolo[3,2-c]pyridin-4-one (28.9 mg, 27.66%) as a light yellow solid.

LC-MS: (M+H)⁺ found: 464.95.

¹H NMR (400 MHz, DMSO-d6) δ 11.55 (s, 1H), 8.53 (d, J=2.8 Hz, 1H), 8.29 (dd, J=5.0, 1.1 Hz, 1H), 7.54 (s, 1H), 7.49 (dd, J=6.8, 5.1 Hz, 1H), 7.35-6.96 (m, 2H), 6.86-6.79 (m, 1H), 6.58 (ddd, J=9.9, 8.3, 1.4 Hz, 1H), 6.13 (dd, J=8.4, 1.6 Hz, 1H), 3.56-3.43 (m, 3H), 3.31 (s, 3H), 3.26-3.20 (m, 1H), 3.09 (dt, J=8.3, 5.4 Hz, 1H), 2.00 (dp, J=17.5, 6.2, 5.6 Hz, 1H), 1.78 (ddt, J=13.9, 8.3, 5.6 Hz, 1H).

### Example 64. (7S)-3-{[2-(difluoromethoxy)-3-fluorophenyl]amino}-2-(3-fluoropyridin-4-yl)-7-(2-methoxyethyl)-1H,5H,6H,7H-pyrrolo[3,2-c]pyridin-4-one (compound 471)

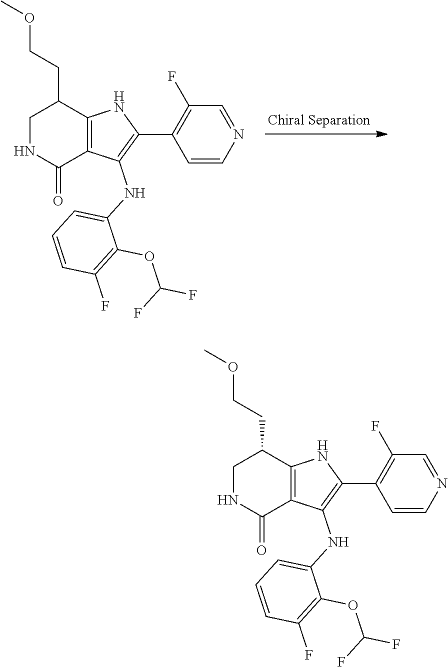 [SMILES: COCCC1CNC(=O)c2c1[nH]c(-c1ccncc1F)c2Nc1cccc(F)c1OC(F)F ; COCC[C@H]1CNC(=O)c2c1[nH]c(-c1ccncc1F)c2Nc1cccc(F)c1OC(F)F]

The crude product 3-{[2-(difluoromethoxy)-3-fluorophenyl]amino}-2-(3-fluoropyridin-4-yl)-7-(2-methoxyethyl)-1H,5H,6H,7H-pyrrolo[3,2-c]pyridin-4-one (100 mg, 0.215 mmol, 1.00 equiv) was purified by Prep-Chiral-HPLC with the following conditions (Column: CHIRAL ART Amylose-SA, 2*25 cm, 5 μm; Mobile Phase A: Hex (0.5% 2M NH3-MeOH)-HPLC, Mobile Phase B: EtOH-HPLC; Flow rate: 20 mL/min; Gradient: 25% B to 25% B in 11 min; Wave Length: 220/254 nm; RT1 (min): 7.57; RT2 (min): 9.68; Sample Solvent: EtOH-HPLC; Injection Volume: 0.8 mL; Number Of Runs: 5) to afford (7S)-3-{[2-(difluoromethoxy)-3-fluorophenyl]amino}-2-(3-fluoropyridin-4-yl)-7-(2-methoxyethyl)-1H,5H,6H,7H-pyrrolo[3,2-c]pyridin-4-one (38.2 mg, 37.93%) as a light yellow solid.

LC-MS: (M+H)⁺ found: 464.95.

¹H NMR (400 MHz, DMSO-d6) δ 11.55 (s, 1H), 8.52 (d, J=2.8 Hz, 1H), 8.29 (dd, J=5.1, 1.1 Hz, 1H), 7.54 (s, 1H), 7.49 (dd, J=6.8, 5.1 Hz, 1H), 7.36-6.97 (m, 2H), 6.84 (td, J=8.4, 6.0 Hz, 1H), 6.58 (ddd, J=9.9, 8.3, 1.4 Hz, 1H), 6.12 (dt, J=8.5, 1.4 Hz, 1H), 3.55-3.43 (m, 3H), 3.24 (ddd, J=12.4, 5.9, 3.1 Hz, 1H), 3.08 (dq, J=11.2, 5.5 Hz, 1H), 2.01 (dq, J=13.1, 6.7 Hz, 1H), 1.84-1.71 (m, 1H).

### Example 65. (7R)-3-[(3-chloro-2-methoxyphenyl)amino]-7-[(2S)-1,4-dioxan-2-ylmethyl]-2-(3-fluoropyridin-4-yl)-1H,5H,6H,7H-pyrrolo[3,2-c]pyridin-4-one (compound 483)

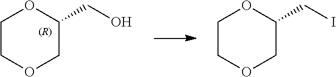

To a stirred solution of 1,4-dioxan-2-ylmethanol (1400 mg, 11.851 mmol, 1.00 equiv) in Toluene (70.0 mL) was added 1H-imidazole (1678.13 mg, 24.650 mmol, 2.08 equiv) and 12 (3158.32 mg, 12.444 mmol, 1.05 equiv) at rt. After stirring the solution at rt for 1 h under N2 atmosphere, THF (35.00 mL) was added. Then, the solution was stirred at rt for 2 h under N2 atmosphere. The reaction was quenched with NaS2O3 at rt and extracted with 3×20 ml of diethyl ether. The extracts were washed with brine (1×30 ml), dried over sodium sulfate, and concentrated under reduced pressure. The residue was purified by silica gel column chromatography, eluted with PE:EA=6:1 to afford 2-(iodomethyl)-1,4-dioxane (1150 mg, 42.55%) as a white oil.

LC-MS: M−56 found: 228.85.

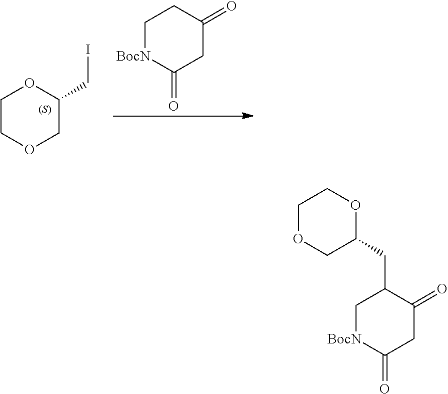

To a stirred solution of tert-butyl 2,4-dioxopiperidine-1-carboxylate (600 mg, 2.814 mmol, 1.00 equiv) in THF (18.00 mL) were added LDA (904.28 mg, 8.442 mmol, 3 equiv) at −80 degrees C. under N2 atmosphere. Then, then the solution was added 2-(iodomethyl)-1,4-dioxane (769.96 mg, 3.377 mmol, 1.2 equiv) at −80 degrees C. under N2 atmosphere. The solution was stirred at rt for 16 h. The mixture was acidified to pH 3-4 with HCl. The residue was washed with EA (3×40 ml). The extracts was concentrated under reduced pressure and purified by Prep-TLC (PE:EA=1:1) by 3 times to afford tert-butyl 5-(1,4-dioxan-2-ylmethyl)-2,4-dioxopiperidine-1-carboxylate (230 mg, 26.09%) as a yellow solid.

LC-MS: M−56 found: 257.95.

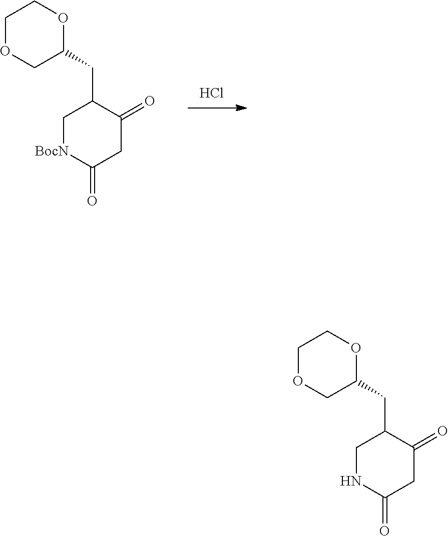

To a stirred solution of tert-butyl 5-(1,4-dioxan-2-ylmethyl)-2,4-dioxopiperidine-1-carboxylate (690 mg, 2.202 mmol, 1.00 equiv) in DCM (15 mL) was added HCl (gas) in 1,4-dioxane (7.89 mL, 259.528 mmol, 117.86 equiv) at rt under N2 atmosphere. Then, the solution was stirred at rt for 2 h. The resulting mixture was concentrated under reduced pressure to afford crude 5-(1,4-dioxan-2-ylmethyl)piperidine-2,4-dione (470 mg, 100.10%) as a yellow oil.

LC-MS: M+41 found: 254.2.

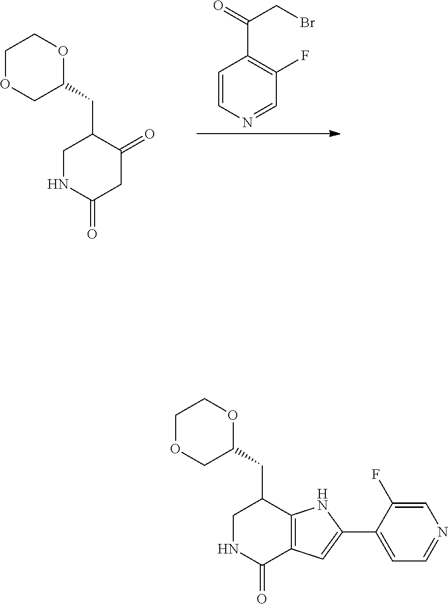

To a stirred solution of 5-(1,4-dioxan-2-ylmethyl)piperidine-2,4-dione (470 mg, 2.204 mmol, 1.00 equiv) and NH₄OAc (849.51 mg, 11.020 mmol, 5.0 equiv) in EtOH (13 mL) were added 2-bromo-1-(3-fluoropyridin-4-yl)ethanone (480.56 mg, 2.204 mmol, 1.0 equiv) at rt under N2 atmosphere. Then, the solution was stirred at 50 degrees C. for 16 h. The resulting mixture was concentrated under reduced pressure and purified by Prep-TLC (DCM:MeOH=12:1) to afford 7-(1,4-dioxan-2-ylmethyl)-2-(3-fluoropyridin-4-yl)-1H,5H,6H,7H-pyrrolo[3,2-c]pyridin-4-one (620 mg, 84.89%) as a pink solid.

LC-MS: M+H found: 332.1.

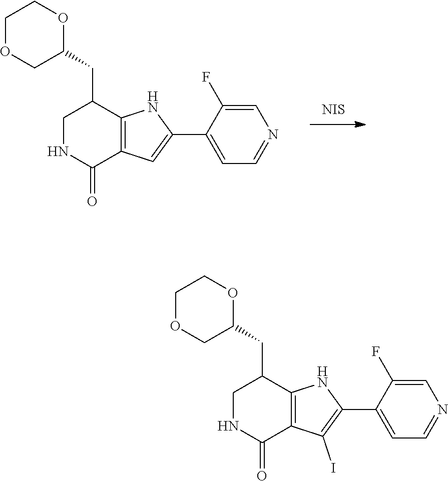

To a stirred solution of 7-(1,4-dioxan-2-ylmethyl)-2-(3-fluoropyridin-4-yl)-1H,5H,6H,7H-pyrrolo[3,2-c]pyridin-4-one (375 mg, 1.132 mmol, 1.00 equiv) in DMF (14 mL) was added NIS (305.55 mg, 1.358 mmol, 1.2 equiv) at rt under N2 atmosphere. Then, the solution was stirred at rt for 1 h. The resulting mixture was concentrated under reduced pressure and purified by Prep-TLC (DCM:MeOH=15:1) to afford 7-(1,4-dioxan-2-ylmethyl)-2-(3-fluoropyridin-4-yl)-3-iodo-1H,5H,6H,7H-pyrrolo[3,2-c]pyridin-4-one (485 mg, 93.72%) as a yellow solid.

LC-MS: M+H found: 458.0.

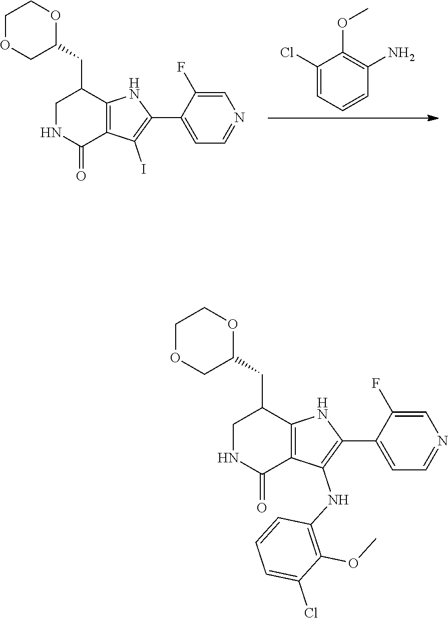

To a stirred solution of 7-(1,4-dioxan-2-ylmethyl)-2-(3-fluoropyridin-4-yl)-3-iodo-1H,5H,6H,7H-pyrrolo[3,2-c]pyridin-4-one (110 mg, 0.241 mmol, 1.00 equiv) and Cs₂CO₃ (36.63 mg, 0.482 mmol, 2.0 equiv) in DMF (4.40 mL) was added EPhos Pd G4 (44.20 mg, 0.048 mmol, 0.2 equiv) and 3-chloro-2-methoxyaniline (113.74 mg, 0.723 mmol, 3.0 equiv) at rt under N2 atmosphere. Then, the solution was stirred at 50 degrees C. for 2 h. The resulting mixture was concentrated under reduced pressure and purified by Prep-TLC (DCM:MeOH=20:1) to afford 3-[(3-chloro-2-methoxyphenyl)amino]-7-(1,4-dioxan-2-ylmethyl)-2-(3-fluoropyridin-4-yl)-1H,5H,6H,7H-pyrrolo[3,2-c]pyridin-4-one (68 mg, 58.05%) as a yellow oil.

LC-MS: M+H found: 487.1.

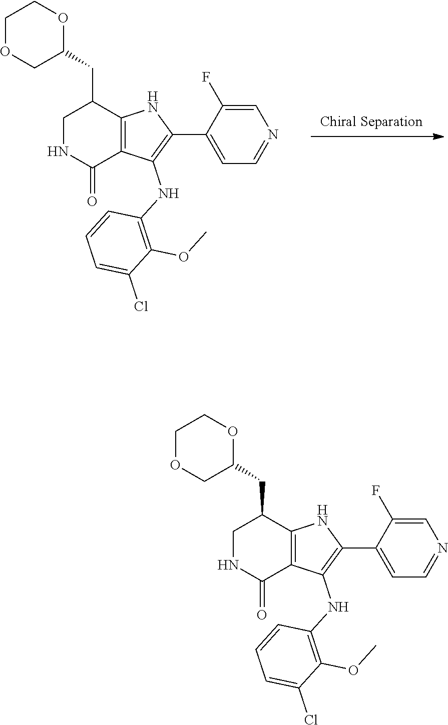

The 3-[(3-chloro-2-methoxyphenyl)amino]-7-(1,4-dioxan-2-ylmethyl)-2-(3-fluoropyridin-4-yl)-1H,5H,6H,7H-pyrrolo[3,2-c]pyridin-4-one (103 mg, 0.212 mmol, 1.00 equiv) was purified by reverse flash chromatography with the following conditions: Column: YMC-Actus Triart C18, 30*150 mm, 5 μm; Mobile Phase A: Water (10 mmol/L NH4HCO3+0.1% NH3·H2O), Mobile Phase B: ACN; Flow rate: 60 mL/min; Gradient: 25% B to 45% B in 10 min, 45% B; Wave Length: 254/220 nm; RT1 (min): 10.05 to afford (R)-7-(((R)-1,4-dioxan-2-yl)methyl)-3-((3-chloro-2-methoxyphenyl)amino)-2-(3-fluoropyridin-4-yl)-1,5,6,7-tetrahydro-4H-pyrrolo[3,2-c]pyridin-4-one (26.4 mg, 25.63%) as a yellow solid.

LC-MS: M+H found: 487.0.

¹H NMR (400 MHz, DMSO-d6) δ 11.45 (s, 1H), 8.51 (d, J=2.8 Hz, 1H), 8.30 (d, J=5.1 Hz, 1H), 7.57 (s, 1H), 7.45 (dd, J=6.8, 5.1 Hz, 1H), 7.18 (s, 1H), 6.70-6.60 (m, 2H), 6.13 (dd, J=7.1, 2.5 Hz, 1H), 3.86 (s, 3H), 3.77 (dd, J=10.5, 2.3 Hz, 1H), 3.73-3.53 (m, 5H), 3.53-3.42 (m, 1H), 3.28 (q, J=4.3 Hz, 1H), 3.26-3.15 (m, 1H), 3.10 (dd, J=8.1, 5.1 Hz, 1H), 1.76 (dt, J=14.1, 5.0 Hz, 1H), 1.60 (dt, J=14.6, 8.0 Hz, 1H).

### Example 66. (S)-7-(((R)-1,4-dioxan-2-yl)methyl)-3-((3-chloro-2-methoxyphenyl)amino)-2-(3-fluoropyridin-4-yl)-1,5,6,7-tetrahydro-4H-pyrrolo[3,2-c]pyridin-4-one (compound 482)

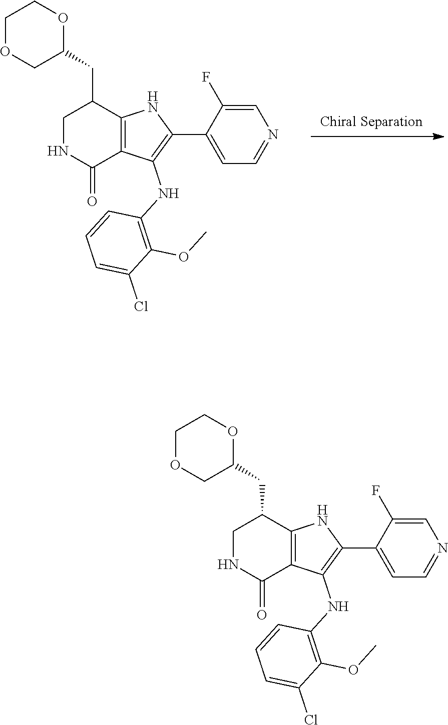

The 3-[(3-chloro-2-methoxyphenyl)amino]-7-(1,4-dioxan-2-ylmethyl)-2-(3-fluoropyridin-4-yl)-1H,5H,6H,7H-pyrrolo[3,2-c]pyridin-4-one (103 mg, 0.212 mmol, 1.00 equiv) was purified by reverse flash chromatography with the following conditions: Column: YMC-Actus Triart C8, 20*250 mm, 5 μm; Mobile Phase A: Water (10 mmol/L NH4HCO3+0.1% NH3·H2O), Mobile Phase B: ACN; Flow rate: 60 mL/min; Gradient: 25% B to 45% B in 10 min, 45% B; Wave Length: 254/220 nm; RT1 (min): 9.56 to afford (S)-7-(((R)-1,4-dioxan-2-yl)methyl)-3-((3-chloro-2-methoxyphenyl)amino)-2-(3-fluoropyridin-4-yl)-1,5,6,7-tetrahydro-4H-pyrrolo[3,2-c]pyridin-4-one (23.1 mg, 22.43%) as a yellow solid.

LC-MS: M+H found: 487.0.

¹H NMR (400 MHz, DMSO-d6) δ 11.52 (s, 1H), 8.50 (d, J=2.9 Hz, 1H), 8.29 (d, J=5.1 Hz, 1H), 7.59 (s, 1H), 7.44 (dd, J=6.8, 5.1 Hz, 1H), 7.18 (d, J=3.2 Hz, 1H), 6.72-6.61 (m, 2H), 6.13 (dd, J=7.4, 2.3 Hz, 1H), 3.84-3.74 (m, 2H), 3.68 (ddt, J=18.8, 11.3, 5.6 Hz, 4H), 3.50 (t, J=10.5 Hz, 2H), 3.33 (s, 1H), 3.29-3.19 (m, 3H), 3.18 (d, J=5.0 Hz, 1H), 1.79 (ddd, J=14.1, 9.5, 4.9 Hz, 1H), 1.59 (ddd, J=13.9, 8.6, 3.0 Hz, 1H).

### Example 67. (7S)-3-[(3-chloro-2-methoxyphenyl)amino]-7-[(2S)-1,4-dioxan-2-ylmethyl]-2-(3-fluoropyridin-4-yl)-1H,5H,6H,7H-pyrrolo[3,2-c]pyridin-4-one (compound 481)

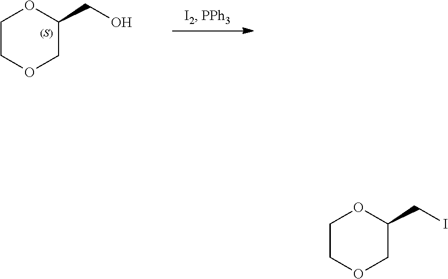

I₂ (6.78 g, 26.713 mmol, 1.05 equiv), Ph₃P (7.02 g, 26.765 mmol, 1.05 equiv), and imidazole (3.54 g, 52.000 mmol, 2.06 equiv) were added to a solution of 1,4-dioxan-2-ylmethanol (3 g, 25.4 mmol, 1.00 equiv) in Toluene (120 mL). After stirring for 10 min at rt, THF (60 mL) was added, and the solution was allowed to stir 10h. The resulting solution was quenched with saturated sodium thiosulfate solution (120 mL) and extracted with diethyl ether (3×120 mL). The extracts were washed with brine (180 mL), dried over sodium sulfate, and concentrated under reduced pressure. The residue was extracted with ether-hexane (18 ml: 120 mL) to remove solid triphenylphosphine oxide. The extract was concentrated and purified by column chromatography (PE:EA=6:1) to give a clear liquid 2-(iodomethyl)-1,4-dioxane (2.8 g, 48.27%) as a white oil.

GC-MS: M+H found: 228.

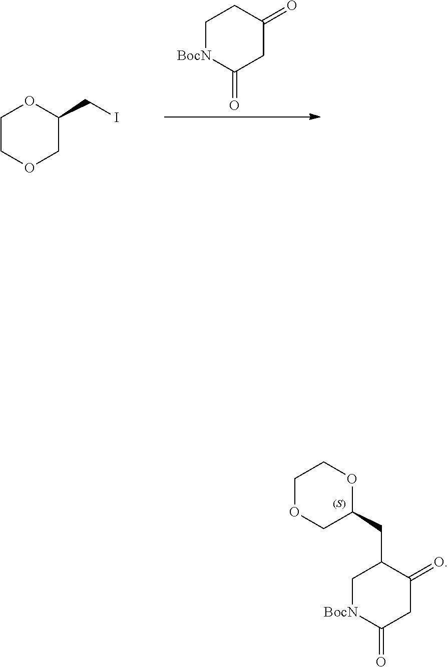

A solution of tert-butyl 2,4-dioxopiperidine-1-carboxylate (800 mg, 3.752 mmol, 1.00 equiv) in THF (24 mL) was treated under nitrogen atmosphere, when the temperature was got −70 degrees C., followed by the addition of LDA (5.6 mL, 41.298 mmol) dropwise at −70 degrees C. and remained the temperature about 30 min, then added the (2R)-2-(iodomethyl)-1,4-dioxane (1026.61 mg, 4.502 mmol, 1.20 equiv), finally the mixture was stirred at rt about 16 h. The reaction was quenched by the addition of H2O (5 mL) at RT. The mixture was acidified to pH 3 with HCl. The resulting mixture was extracted with EA (3×15 mL). The combined organic layers were washed with saturated salt solution (3×15 mL), dried over anhydrous sodium sulfate. After filtration, the filtrate was concentrated under reduced pressure. The residue was purified by Prep-TLC (PE:EA=1:1) to afford tert-butyl 5-(1,4-dioxan-2-ylmethyl)-2,4-dioxopiperidine-1-carboxylate (306 mg, 26.03%) as a yellow solid.

LC-MS: 2M+Na found: 649.5.

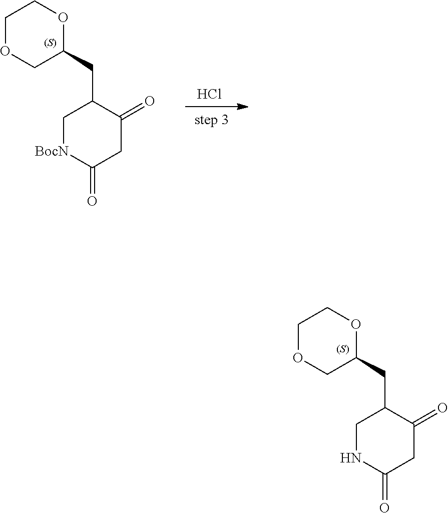

To a stirred solution of tert-butyl 5-(1,4-dioxan-2-ylmethyl)-2,4-dioxopiperidine-1-carboxylate (306 mg, 0.977 mmol, 1.00 equiv) in DCM (3.6 mL) was added HCl (gas) in 1,4-dioxane (1.8 mL, 59.241 mmol, 60.66 equiv) at rt. Then the solution was stirred at rt about 2 h. The resulting mixture was concentrated under reduced pressure and get the 5-(1,4-dioxan-2-ylmethyl)piperidine-2,4-dione (200 mg, 96.05%) as a yellow oil.

LC-MS: M+H found: 214.2.

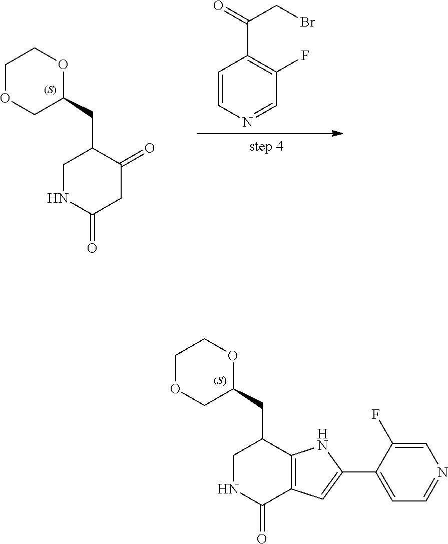

To a stirred solution of 5-(1,4-dioxan-2-ylmethyl)piperidine-2,4-dione (306 mg, 1.435 mmol, 1.00 equiv) and 2-bromo-1-(3-fluoropyridin-4-yl)ethanone (375.45 mg, 1.722 mmol, 1.2 equiv) in EtOH (5.98 mL) were added NH4OAc (553.09 mg, 7.175 mmol, 5.00 equiv) at rt. Then the solution was stirred at 50 degrees C. about 16 h. The residue was purified by Prep-TLC (MeOH:DCM=20:1) to afford 7-(1,4-dioxan-2-ylmethyl)-2-(3-fluoropyridin-4-yl)-1H,5H,6H,7H-pyrrolo[3,2-c]pyridin-4-one (340 mg, 71.50%) as a yellow solid.

LC-MS: M+H found: 332.0.

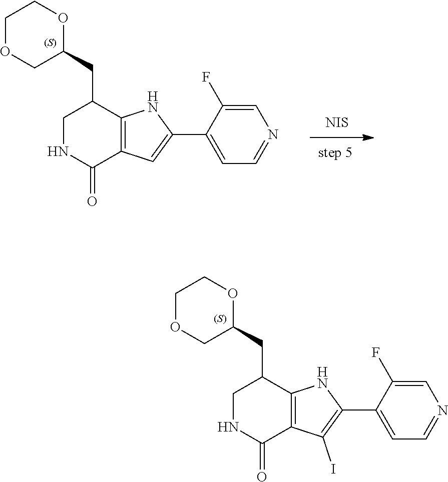

To a stirred solution of 7-(1,4-dioxan-2-ylmethyl)-2-(3-fluoropyridin-4-yl)-1H,5H,6H,7H-pyrrolo[3,2-c]pyridin-4-one (270 mg, 0.815 mmol, 1.00 equiv) in DMF (5.40 mL) was added (iodoamino)sulfanyl (211.42 mg, 1.222 mmol, 1.5 equiv) at rt, then the solution was stirred at rt about 2 h. The resulting mixture was concentrated under reduced pressure and then was purified by Prep-TLC (DCM:MeOH=20:1) to afford 7-(1,4-dioxan-2-ylmethyl)-2-(3-fluoropyridin-4-yl)-3-iodo-1H,5H,6H,7H-pyrrolo[3,2-c]pyridin-4-one (230 mg, 61.73%) as a yellow solid.

LC-MS: M+H found: 457.9.

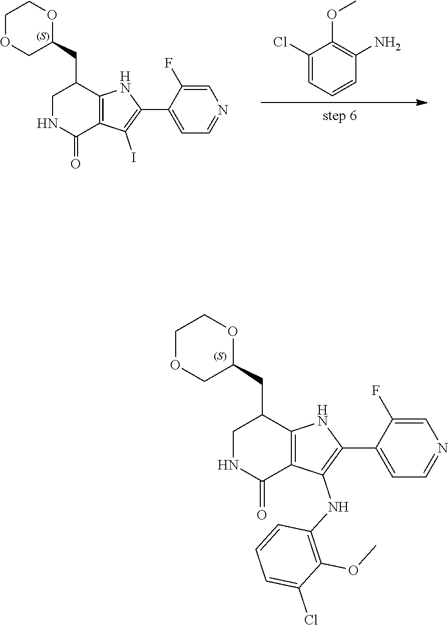

To a stirred solution of 7-(1,4-dioxan-2-ylmethyl)-2-(3-fluoropyridin-4-yl)-3-iodo-1H,5H,6H,7H-pyrrolo[3,2-c]pyridin-4-one (235 mg, 0.514 mmol, 1.00 equiv) and 3-chloro-2-methoxyaniline (242.99 mg, 1.542 mmol, 3 equiv) in DMF (5.0 mL) were added Cs₂CO₃ (334.91 mg, 1.028 mmol, 2.00 equiv) and EPhos Pd G4 (94.42 mg, 0.103 mmol, 0.2 equiv) at rt, then the solution was stirred at 50 degrees C. under N2 atmosphere about 3 h. The resulting mixture was extracted with EA (3×20 mL). The combined organic layers were washed with saturated salt solution (3×20 mL), dried over anhydrous sodium sulfate. After filtration, the filtrate was concentrated under reduced pressure. The residue was purified by Prep-TLC (DCM:MeOH=20:1) to afford 3-[(3-chloro-2-methoxyphenyl) amino]-7-(1,4-dioxan-2-ylmethyl)-2-(3-fluoropyridin-4-yl)-1H,5H,6H,7H-pyrrolo[3,2-c]pyridin-4-one (130 mg, 51.95%) as a yellow solid.

LC-MS: M+H found: 487.3.

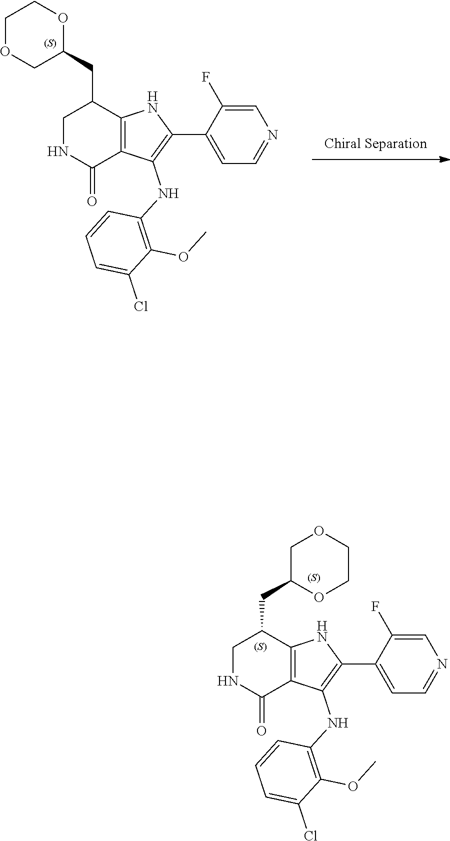

The 3-[(3-chloro-2-methoxyphenyl)amino]-7-(1,4-dioxan-2-ylmethyl)-2-(3-fluoropyridin-4-yl)-1H,5H,6H,7H-pyrrolo[3,2-c]pyridin-4-one (140 mg, 0.288 mmol, 1.00 equiv) was purified by reverse flash chromatography with the following conditions: Column: YMC-Actus Triart C18, 30*150 mm, 5 μm, Mobile Phase A: Water (10 mmol/L NH4HCO3+0.1% NH3·H2O), Mobile Phase B: ACN; Flow rate: 60 mL/min; Gradient: 35% B to 55% B in 8 min, 55% B; Wave Length: 254/220 nm; RT1 (min): 7.10/8.48 to afford (7S)-3-[(3-chloro-2-methoxyphenyl)amino]-7-[(2S)-1,4-dioxan-2-ylmethyl]-2-(3-fluoropyridin-4-yl)-1H,5H,6H,7H-pyrrolo[3,2-c]pyridin-4-one (11.2 mg, 7.94%) as a yellow solid.

LC-MS: M+H found: 486.9.

¹H NMR (400 MHz, DMSO-d6) δ 11.45 (s, 1H), 8.51 (d, J=2.8 Hz, 1H), 8.30 (d, J=5.1 Hz, 1H), 7.57 (s, 1H), 7.49-7.41 (m, 1H), 7.18 (s, 1H), 6.72-6.62 (m, 2H), 6.13 (dd, J=7.1, 2.6 Hz, 1H), 3.81-3.66 (m, 3H), 3.66-3.33 (m, 7H), 3.30-3.09 (m, 3H), 1.77 (dd, J=14.2, 5.3 Hz, 1H), 1.60 (dt, J=15.0, 8.1 Hz, 1H).

### Example 68. (7R)-3-[(3-chloro-2-methoxyphenyl)amino]-7-[(2R)-1,4-dioxan-2-ylmethyl]-2-(3-fluoropyridin-4-yl)-1H,5H,6H,7H-pyrrolo[3,2-c]pyridin-4-one (compound 480)

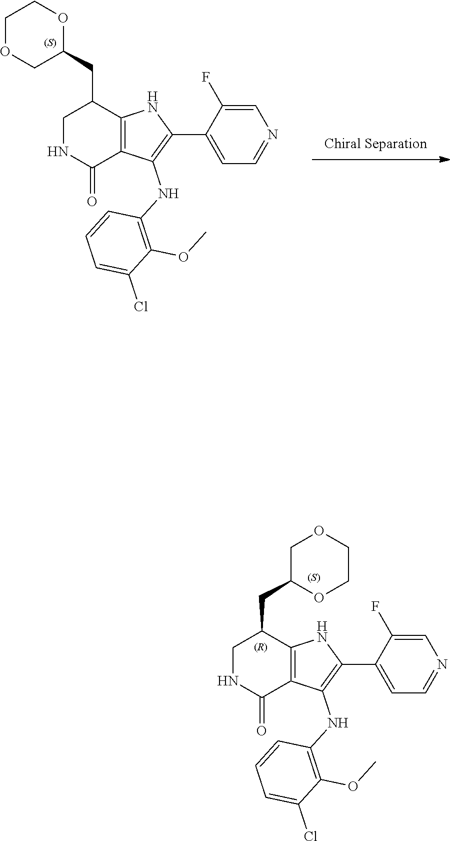

The 3-[(3-chloro-2-methoxyphenyl)amino]-7-(1,4-dioxan-2-ylmethyl)-2-(3-fluoropyridin-4-yl)-1H,5H,6H,7H-pyrrolo[3,2-c]pyridin-4-one (140 mg, 0.288 mmol, 1.00 equiv) was purified by reverse flash chromatography with the following conditions: Column: YMC-Actus Triart C18, 30*150 mm, 5 μm; Mobile Phase A: Water (10 mmol/L NH4HCO3+0.1% NH3·H2O), Mobile Phase B: ACN; Flow rate: 60 mL/min; Gradient: 35% B to 55% B in 8 min, 55% B; Wave Length: 254/220 nm; RT1 (min): 7.10/8.48 to afford (7R)-3-[(3-chloro-2-methoxyphenyl)amino]-7-[(2R)-1,4-dioxan-2-ylmethyl]-2-(3-fluoropyridin-4-yl)-1H,5H,6H,7H-pyrrolo[3,2-c]pyridin-4-one (12.1 mg, 8.46%) as a yellow solid.

LC-MS: M+H found: 486.9.

¹H NMR (400 MHz, DMSO-d6) δ 11.45 (s, 1H), 8.51 (d, J=2.8 Hz, 1H), 8.30 (d, J=5.1 Hz, 1H), 7.57 (s, 1H), 7.49-7.41 (m, 1H), 7.18 (s, 1H), 6.72-6.62 (m, 2H), 6.13 (dd, J=7.1, 2.6 Hz, 1H), 3.81-3.66 (m, 3H), 3.66-3.33 (m, 7H), 3.30-3.09 (m, 3H), 1.77 (dd, J=14.2, 5.3 Hz, 1H), 1.60 (dt, J=15.0, 8.1 Hz, 1H).

### Example 69. methyl 3′-[(3-chloro-2-methoxyphenyl)amino]-2′-(3-fluoropyridin-4-yl)-4′-oxo-5′,6′-dihydro-1′H-spiro[piperidine-4,7′-pyrrolo[3,2-c]pyridine]-1-carboxylate (compound 479)

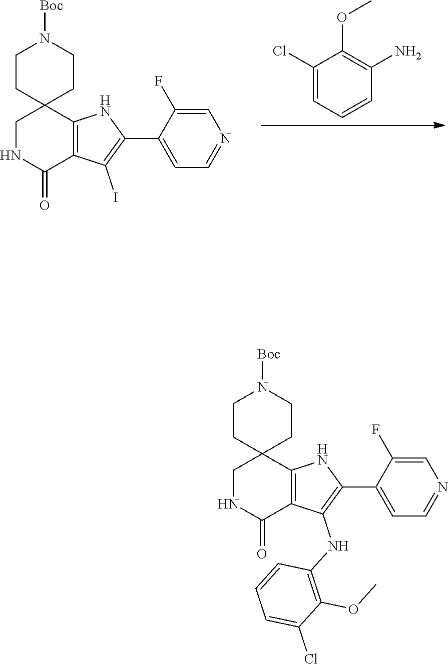

To a stirred solution of tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (500 mg, 0.950 mmol, 1.00 equiv) and 3-chloro-2-methoxyaniline (449.13 mg, 2.850 mmol, 3 equiv) in DMF (4 mL) were added Cs₂CO₃ (619.02 mg, 1.900 mmol, 2 equiv) and EPhos Pd G4 (174.51 mg, 0.190 mmol, 0.2 equiv) at rt, then the solution was stirred at 50 degrees C. under N2 atmosphere about 2 h. The resulting mixture was extracted with EA (3×20 mL). The combined organic layers were washed with saturated salt solution (3×20 mL), dried over anhydrous sodium sulfate. After filtration, the filtrate was concentrated under reduced pressure. The residue was purified by Prep-TLC (DCM:MeOH=20:1) to afford tert-butyl 3′-[(3-chloro-2-methoxyphenyl)amino]-2′-(3-fluoropyridin-4-yl)-4′-oxo-5′,6′-dihydro-1′H-spiro[piperidine-4,7′-pyrrolo[3,2-c]pyridine]-1-carboxylate (332 mg, 62.85%) as a yellow solid.

GC-MS: M+H found: 556.1.

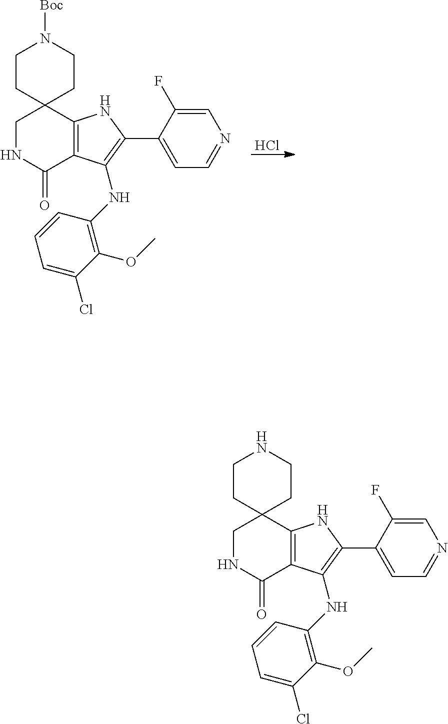 [SMILES: COc1c(Cl)cccc1Nc1c(-c2ccncc2F)[nH]c2c1C(=O)NCC21CCN(C(=O)OC(C)(C)C)CC1 ; Cl ; COc1c(Cl)cccc1Nc1c(-c2ccncc2F)[nH]c2c1C(=O)NCC21CCNCC1]

A solution of tert-butyl 2,4-dioxopiperidine-1-carboxylate (800 mg, 3.752 mmol, 1.00 equiv) in THF (24 mL) was treated under nitrogen atmosphere, when the temperature was got −70 degrees C., followed by the addition of LDA (5.6 mL, 41.298 mmol) dropwise at −70 degrees C. and remained the temperature about 30 min, then added the (2R)-2-(iodomethyl)-1,4-dioxane (1026.61 mg, 4.502 mmol, 1.20 equiv), finally the mixture was stirred at rt about 16 h. The reaction was quenched by the addition of H2O (5 mL) at RT. The mixture was acidified to pH 3 with HCl. The resulting mixture was extracted with EA (3×15 mL). The combined organic layers were washed with saturated salt solution (3×15 mL), dried over anhydrous sodium sulfate. After filtration, the filtrate was concentrated under reduced pressure. The residue was purified by Prep-TLC (PE:EA=1:1) to afford tert-butyl 5-(1,4-dioxan-2-ylmethyl)-2,4-dioxopiperidine-1-carboxylate (306 mg, 26.03%) as a yellow solid.

LC-MS: M+H found: 455.9.

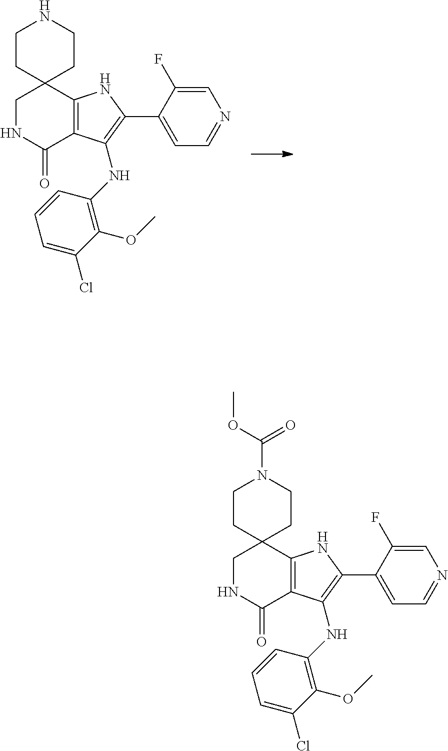 [SMILES: COc1c(Cl)cccc1Nc1c(-c2ccncc2F)[nH]c2c1C(=O)NCC21CCNCC1 ; COC(=O)N1CCC2(CC1)CNC(=O)c1c2[nH]c(-c2ccncc2F)c1Nc1cccc(Cl)c1OC]

To a stirred solution of 3′-[(3-chloro-2-methoxyphenyl)amino]-2′-(3-fluoropyridin-4-yl)-5′,6′-dihydro-1′H-spiro[piperidine-4,7′-pyrrolo[3,2-c]pyridin]-4′-one (400 mg, 0.877 mmol, 1.00 equiv) and Et3N (177.56 mg, 1.754 mmol, 2 equiv) in DCM (5 mL) were added methyl chloroformate (99.48 mg, 1.052 mmol, 1.2 equiv) at rt, then the solution was stirred at rt about 2 h. The residue was purified by Prep-TLC (DCM:MeOH=15:1) to afford crude product as a yellow solid. The residue was purified by reverse flash chromatography with the following conditions: Column: XBridge Prep OBD C18 Column, 30*150 mm, 5 μm; Mobile Phase A: Water (10 mmol/L NH4HCO3+0.1% NH3·H2O), Mobile Phase B: ACN; Flow rate: 60 mL/min; Gradient: 26% B to 56% B in 8 min, 56% B; Wave Length: 254; 220 nm; RT1 (min): 7.25; Number Of Runs: to afford methyl 3′-[(3-chloro-2-methoxyphenyl)amino]-2′-(3-fluoropyridin-4-yl)-4′-oxo-5′,6′-dihydro-1′H-spiro[piperidine-4,7′-pyrrolo[3,2-c]pyridine]-1-carboxylate (53.7 mg, 11.85%) as a yellow solid.

LC-MS: M+H found: 514.0.

¹H NMR (400 MHz, DMSO-d6) δ 11.38 (s, 1H), 8.47 (d, J=2.6 Hz, 1H), 8.32 (dd, J=5.0, 1.2 Hz, 1H), 7.67 (s, 1H), 7.45 (dd, J=6.7, 5.0 Hz, 1H), 7.30 (s, 1H), 6.73-6.52 (m, 2H), 6.09 (dd, J=7.9, 1.8 Hz, 1H), 3.84 (s, 5H), 3.62 (s, 3H), 3.48 (d, J=2.8 Hz, 2H), 3.02 (s, 2H), 1.98 (s, 2H), 1.70 (d, J=13.3 Hz, 2H).

### Example 70. 3′-[(3-chloro-2-methoxyphenyl)amino]-1-(prop-2-enoyl)-2′-(pyrimidin-4-yl)-5′,6′-dihydro-1′H-spiro[piperidine-4,7′-pyrrolo[3,2-c]pyridin]-4′-one (compound 478)

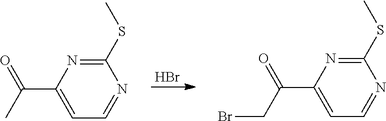

To a stirred solution/mixture of 1-[2-(methylsulfanyl)pyrimidin-4-yl]ethanone (1000 mg, 5.945 mmol, 1.00 equiv) in AcOH/HBr (0.6 mL; 0.5:0.1) were added Br2 (950.05 mg, 5.945 mmol, 1.00 equiv) in AcOH (0.1 mL). The resulting mixture was stirred for 2.5H at 60° C. under nitrogen atmosphere. The resulting mixture was wished with EA. The precipitated solids were collected by filtration and washed with EA (3×5 mL). to afford 2-bromo-1-[2-(methylsulfanyl)pyrimidin-4-yl]ethanone (1100 mg, 74.88%) as a yellow solid.

LC-MS: M+H found: 247.

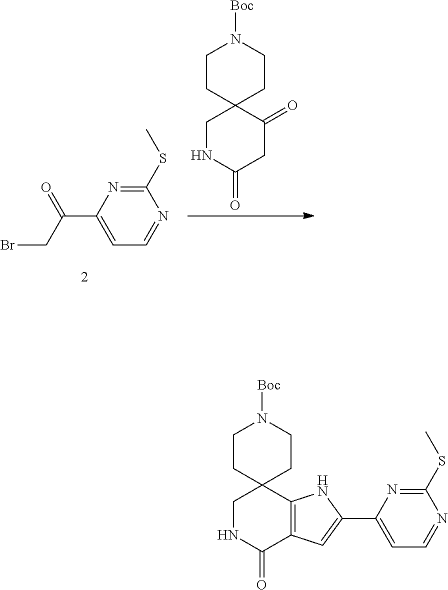

To a stirred solution/mixture of 2-bromo-1-[2-(methylsulfanyl)pyrimidin-4-yl]ethanone (1.1 g, 4.451 mmol, 1 equiv), tert-butyl 3,5-dioxo-2,9-diazaspiro[5.5]undecane-9-carboxylate (1256.82 mg, 4.451 mmol, 1 equiv) and AcONH4 (1715.65 mg, 22.255 mmol, 5 equiv) in EtOH (11 mL). The resulting mixture was stirred for 16 h at 50° C. under nitrogen atmosphere. The reaction was monitored by LCMS. Desired product could be detected by LCMS. The resulting mixture was diluted with MeOH (10 mL). The resulting mixture was filtered; the filter cake was washed with MeOH (2×5 ml). The filter cake was collected to afford tert-butyl 2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (1.1 g, 57.53%) as an off-white solid.

LC-MS: M+H found: 430.

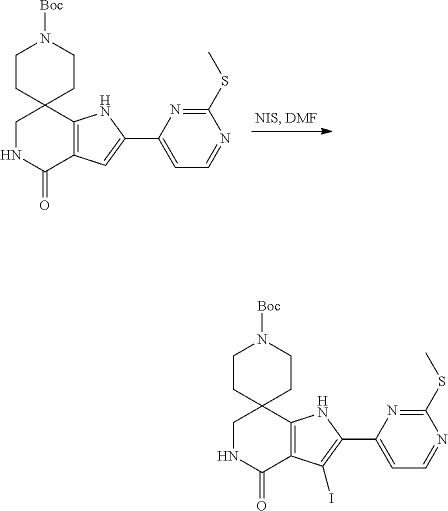 [SMILES: CSc1nccc(-c2cc3c([nH]2)C2(CCN(C(=O)OC(C)(C)C)CC2)CNC3=O)n1 ; CSc1nccc(-c2[nH]c3c(c2I)C(=O)NCC32CCN(C(=O)OC(C)(C)C)CC2)n1]

Into a 40-mL vial, was placed tert-butyl 2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (1.1 g, 2.561 mmol, 1 equiv) and NIS (864.24 mg, 3.841 mmol, 1.5 equiv) in DMF (10 mL). The resulting solution was stirred for 2H at 50 degrees C. The reaction was monitored by LCMS. Desired product could be detected by LCMS. The resulting mixture was diluted with H2O (60 mL). The resulting mixture was filtered; the filter cake was washed with H2O (2×5 ml). The filter cake was collected. The crude product was purified by Prep-Flash (DCM/MEOH: 20:1) to afford tert-butyl 3′-iodo-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (1.1 g, 77.33%) as an off-white solid.

LC-MS: M+H found: 556.

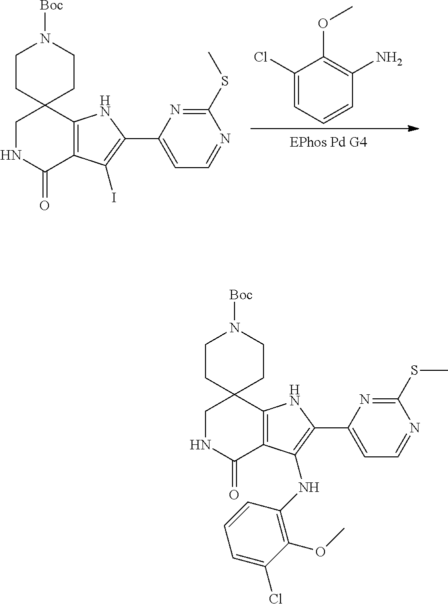

Into a 8-mL vial, was placed tert-butyl 3′-iodo-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (1.1 g, 1.980 mmol, 1 equiv), 3-chloro-2-methoxyaniline (936.34 mg, 5.940 mmol, 3 equiv), EPhos Pd G4 (545.73 mg, 0.594 mmol, 0.3 equiv) and EPhos (635.47 mg, 1.188 mmol, 0.6 equiv)) in DMF (0.5 mL). The resulting solution was stirred for 2 h at 50 degrees C. The reaction was monitored by LCMS. Desired product could be detected by LCMS. The resulting mixture was diluted with H2O (60 mL). The resulting mixture was filtered; the filter cake was washed with H2O (2×5 ml). The filter cake was collected. The crude product was purified by Prep-Flash (DCM/MEOH; 20:1) to afford tert-butyl 3′-[(3-chloro-2-methoxyphenyl)amino]-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (1.0 g) as an off-white solid.

LC-MS: M+H found: 585.

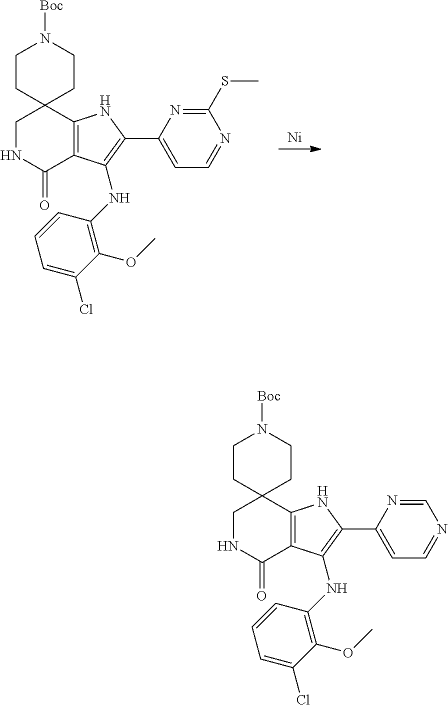

Into a 8-mL vial, was placed tert-butyl 3′-[(3-chloro-2-methoxyphenyl)amino]-2′-[2-(methylsulfanyl)pyrimidin-4-yl]-4′-oxo-5′,6′-dihydro-1′H-spiro[piperidine-4,7′-pyrrolo[3,2-c]pyridine]-1-carboxylate (350 mg, 0.598 mmol, 1 equiv) and Raney Ni (2562.40 mg, 29.900 mmol, 50 equiv) in EtOH (1 mL). The resulting solution was stirred for 2 h at 90 degrees C. The reaction was monitored by LCMS. Desired product could be detected by LCMS. The resulting mixture was diluted with MeOH (10 mL). The resulting mixture was filtered; the filter cake was washed with MeOH (2×5 ml). The filtrate was concentrated under reduced pressure. The crude product was purified by Prep-Flash (DCM/MeOH: 20:1) to afford tert-butyl 3′-[(3-chloro-2-methoxyphenyl)amino]-4′-oxo-2′-(pyrimidin-4-yl)-5′,6′-dihydro-1′H-spiro[piperidine-4,7′-pyrrolo[3,2-c]pyridine]-1-carboxylate (160 mg, 49.62%) as an off-white solid.

LC-MS: M+H found: 539.

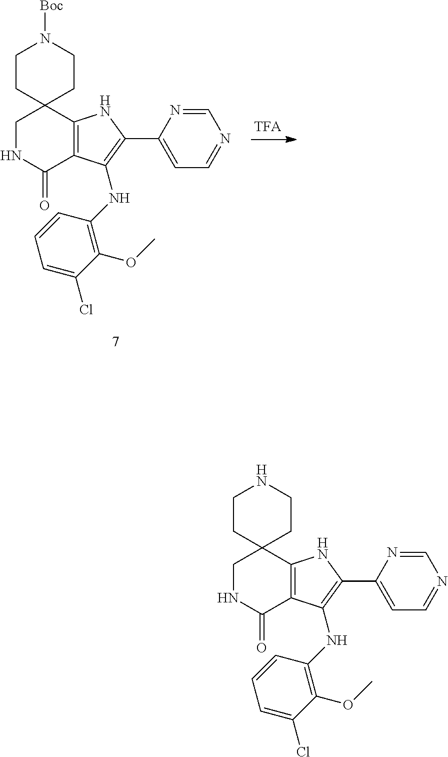 [SMILES: COc1c(Cl)cccc1Nc1c(-c2ccncn2)[nH]c2c1C(=O)NCC21CCN(C(=O)OC(C)(C)C)CC1 ; COc1c(Cl)cccc1Nc1c(-c2ccncn2)[nH]c2c1C(=O)NCC21CCNCC1]

Into a 8-mL vial, was placed tert-butyl 3′-[(3-chloro-2-methoxyphenyl)amino]-4′-oxo-2′-(pyrimidin-4-yl)-5′,6′-dihydro-1′H-spiro[piperidine-4,7′-pyrrolo[3,2-c]pyridine]-1-carboxylate (150 mg, 0.278 mmol, 1 equiv) in DCM (4 mL), then added TFA (1 mL) dropwise. The resulting solution was stirred for 2 h at rt. The reaction was monitored by LCMS. Desired product could be detected by LCMS. The mixture was neutralized to pH 7˜8 with NaHCO3 (aq). The aqueous layer was extracted with EA (3×10 mL). The resulting mixture was concentrated under reduced pressure. The crude product/resulting mixture was used in the next step directly without further purification.

LC-MS: M+H found: 439.

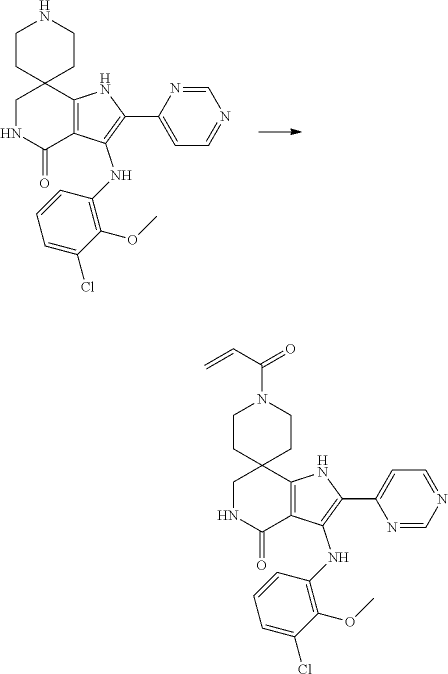 [SMILES: COc1c(Cl)cccc1Nc1c(-c2ccncn2)[nH]c2c1C(=O)NCC21CCNCC1 ; C=CC(=O)N1CCC2(CC1)CNC(=O)c1c2[nH]c(-c2ccncn2)c1Nc1cccc(Cl)c1OC]

Into a 8 mL round-bottom flask were added 3′-[(3-chloro-2-methoxyphenyl)amino]-2′-(pyrimidin-4-yl)-5′,6′-dihydro-1′H-spiro[piperidine-4,7′-pyrrolo[3,2-c]pyridin]-4′-one (150 mg, 0.342 mmol, 1 equiv) in THF/NaHCO3 (aq)(4 ml; 1:1) at 0° C., to the above mixture was added acryloyl chloride (46.40 mg, 0.513 mmol, 1.5 equiv) dropwise/in portions over 1 min at 0° C. The resulting mixture was stirred for additional 15 min at 0° C. The reaction was OK. The aqueous layer was extracted with EA (3×10 mL). The solution was concentrated under reduced pressure. The crude product/resulting mixture got 3′-[(3-chloro-2-methoxyphenyl)amino]-1-(prop-2-enoyl)-2′-(pyrimidin-4-yl)-5′,6′-dihydro-1′H-spiro[piperidine-4,7′-pyrrolo[3,2-c]pyridin]-4′-one (100 mg, 59.36%)

LC-MS: M+H found: 493.

¹H NMR (400 MHz, DMSO-d6) δ 11.82 (s, 1H), 9.03 (d, J=1.4 Hz, 1H), 8.57 (d, J=5.6 Hz, 1H), 8.33 (s, 1H), 7.38-7.27 (m, 2H), 6.93-6.73 (m, 3H), 6.37 (dd, J=6.9, 2.8 Hz, 1H), 6.16 (dd, J=16.7, 2.5 Hz, 1H), 5.72 (dd, J=10.5, 2.5 Hz, 1H), 4.45 (d, J=13.1 Hz, 1H), 4.06 (d, J=13.8 Hz, 1H), 3.92 (s, 3H), 3.52 (d, J=2.9 Hz, 2H), 3.26 (t, J=13.4 Hz, 1H), 2.81 (t, J=13.1 Hz, 1H), 2.14 (d, J=12.9 Hz, 2H), 1.71 (d, J=13.3 Hz, 2H).

### Example 71. (7S)-2-(2-aminopyrimidin-4-yl)-3-[(3-chloro-2-methoxyphenyl)amino]-7-[(2S)-1,4-dioxan-2-ylmethyl]-1H,5H,6H,7H-pyrrolo[3,2-c]pyridin-4-one (compound 531)

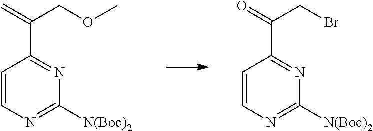

To a stirred solution of tert-butyl N-(tert-butoxycarbonyl)-N-[4-(1-ethoxyethenyl)pyrimidin-2-yl]carbamate (1250 mg, 3.421 mmol, 1.00 equiv) in THF (15 mL) and H2O (1 mL) were added NBS (730.58 mg, 4.105 mmol, 1.2 equiv) at rt under N2 atmosphere. Then, the solution was stirred at rt for 2 h. The resulting mixture was diluted with water (30 ml) and extracted with 3×40 ml of EA. The extract was concentrated under reduce pressure to afford crude tert-butyl N-[4-(2-bromoacetyl)pyrimidin-2-yl]-N-(tert-butoxycarbonyl)carbamate (1300 mg, 91.30%) as a yellow oil.

LC-MS: M+35 found: 450.0.

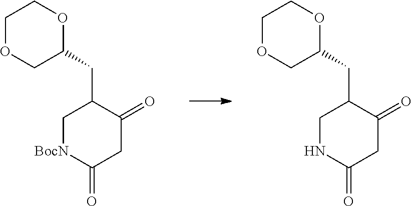

To a stirred solution of tert-butyl 5-(1,4-dioxan-2-ylmethyl)-2,4-dioxopiperidine-1-carboxylate (690 mg, 2.202 mmol, 1.00 equiv) in DCM (15 mL) was added HCl (gas) in 1,4-dioxane (7.89 mL, 259.528 mmol, 117.86 equiv) at rt under N2 atmosphere. Then, the solution was stirred at rt for 2 h. The resulting mixture was concentrated under reduced pressure to afford crude 5-(1,4-dioxan-2-ylmethyl)piperidine-2,4-dione (470 mg, 100.10%) as a yellow oil.

LC-MS: M+15 found: 228.1.

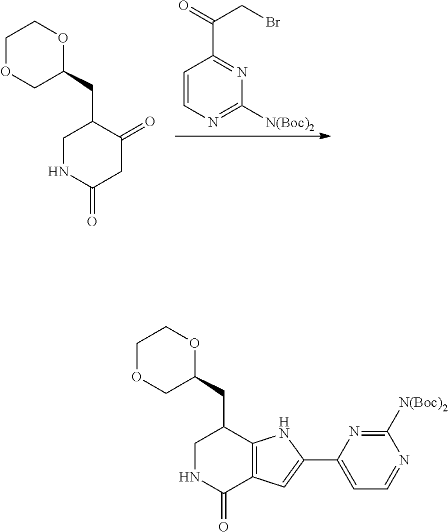

To a stirred solution of 5-[(2R)-1,4-dioxan-2-ylmethyl]piperidine-2,4-dione (680.5 mg, 3.191 mmol, 1.00 equiv) and NH₄OAc (1475.98 mg, 19.146 mmol, 6.0 equiv) in EtOH (10 mL) were added tert-butyl N-[4-(2-bromoacetyl)pyrimidin-2-yl]-N-(tert-butoxycarbonyl)carbamate (1328.47 mg, 3.191 mmol, 1.00 equiv) at rt under N2 atmosphere. Then, the solution was stirred at 50° C. for 16 h. The resulting mixture was concentrated under reduced pressure and purified by silica gel column chromatography, eluted with DCM:MeOH=15:1 to afford tert-butyl N-(tert-butoxycarbonyl)-N-{4-[7-(1,4-dioxan-2-ylmethyl)-4-oxo-1H,5H,6H,7H-pyrrolo[3,2-c]pyridin-2-yl]pyrimidin-2-yl}carbamate (520 mg, 30.77%) as a yellow solid.

LC-MS: M+H found: 530.0.

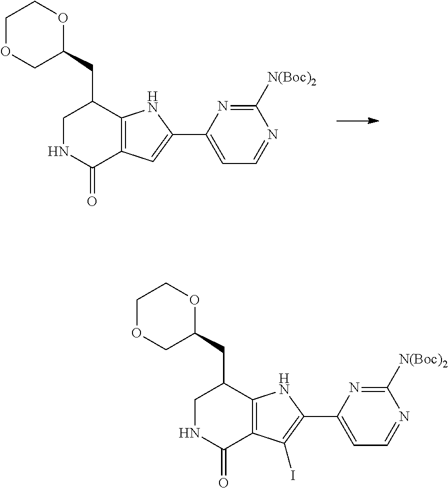

To a stirred solution of tert-butyl N-(tert-butoxycarbonyl)-N-{4-[7-(1,4-dioxan-2-ylmethyl)-4-oxo-1H,5H,6H,7H-pyrrolo[3,2-c]pyridin-2-yl]pyrimidin-2-yl}carbamate (520 mg, 0.982 mmol, 1.00 equiv) in DMF (5 mL) was added NIS (287.18 mg, 1.277 mmol, 1.30 equiv) at RT under N2 atmosphere. Then, the solution was stirred at rt for 2 h. The resulting mixture was concentrated under reduced pressure and purified by Prep-TLC (DCM:MeOH=15:1) to afford tert-butyl N-(tert-butoxycarbonyl)-N-{4-[7-(1,4-dioxan-2-ylmethyl)-3-iodo-4-oxo-1H,5H,6H,7H-pyrrolo[3,2-c]pyridin-2-yl]pyrimidin-2-yl}carbamate (530 mg, 82.35%) as a yellow solid.

LC-MS: M+H found: 656.2.

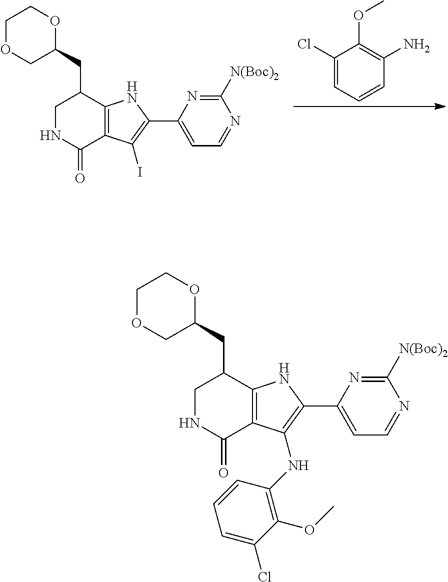

To a stirred solution of tert-butyl N-(tert-butoxycarbonyl)-N-(4-{7-[(2S)-1,4-dioxan-2-ylmethyl]-3-iodo-4-oxo-1H,5H,6H,7H-pyrrolo[3,2-c]pyridin-2-yl}pyrimidin-2-yl)carbamate (400 mg, 0.610 mmol, 1 equiv) and Cs₂CO₃ (397.65 mg, 1.220 mmol, 2.00 equiv) in DMF (1.5 mL) were added 3-chloro-2-methoxyaniline (384.69 mg, 2.441 mmol, 4.00 equiv) and EPhos Pd G4 (112.11 mg, 0.122 mmol, 0.20 equiv) at RT under N2 atmosphere. The solution was stirred at 50° C. for 16 h. The resulting mixture was concentrated under reduced pressure and purified by Prep-TLC (DCM:MeOH=10:1) to afford tert-butyl N-(tert-butoxycarbonyl)-N-(4-{3-[(3-chloro-2-methoxyphenyl)amino]-7-[(2S)-1,4-dioxan-2-ylmethyl]-4-oxo-1H,5H,6H,7H-pyrrolo[3,2-c]pyridin-2-yl}pyrimidin-2-yl)carbamate (350 mg, 83.71%) as a yellow solid.

LC-MS: M+H found: 685.25.

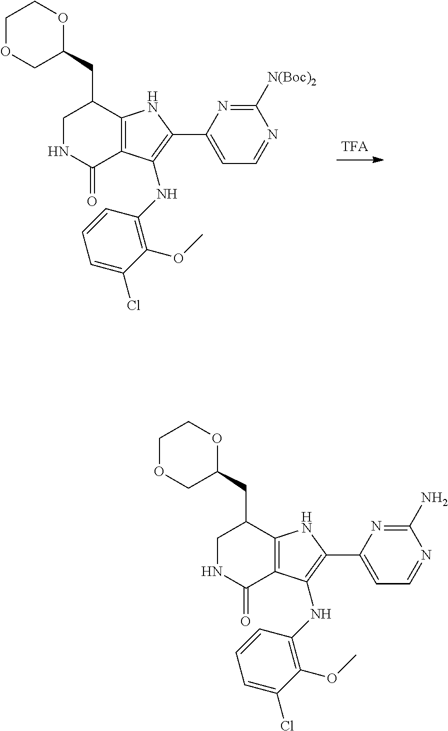

To a stirred solution of tert-butyl N-(tert-butoxycarbonyl)-N-(4-{3-[(3-chloro-2-methoxyphenyl)amino]-7-[(2S)-1,4-dioxan-2-ylmethyl]-4-oxo-1H,5H,6H,7H-pyrrolo[3,2-c]pyridin-2-yl}pyrimidin-2-yl)carbamate (90 mg, 0.131 mmol, 1 equiv) in DCM (0.5 mL) were added TFA (0.75 mL, 10.070 mmol, 76.87 equiv) at RT under N2 atmosphere. Then, the solution was stirred at rt for 30 min. The resulting mixture was concentrated under reduced pressure and purified by Prep-TLC (DCM:MeOH=15:1) to afford 2-(2-aminopyrimidin-4-yl)-3-[(3-chloro-2-methoxyphenyl)amino]-7-[(2R)-1,4-dioxan-2-ylmethyl]-1H,5H,6H,7H-pyrrolo[3,2-c]pyridin-4-one (60 mg, 94.19%) as a light yellow solid.

LC-MS: M+H found: 485.15.

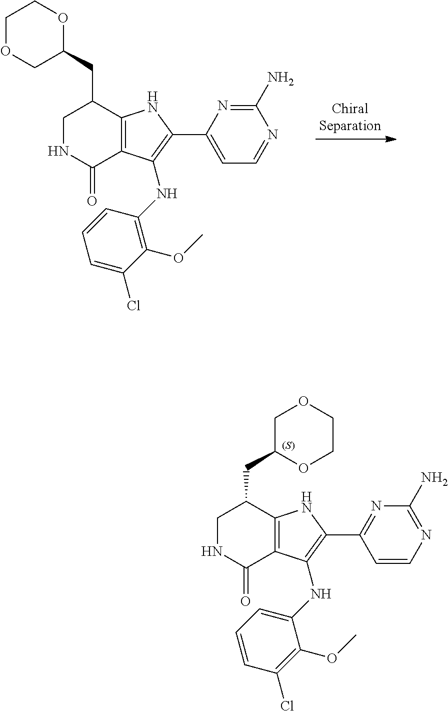

The crude product 2-(2-aminopyrimidin-4-yl)-3-[(3-chloro-2-methoxyphenyl)amino]-7-[(2S)-1,4-dioxan-2-ylmethyl]-1H,5H,6H,7H-pyrrolo[3,2-c]pyridin-4-one (300 mg, 0.619 mmol, 1 equiv) was purified by Prep-HPLC as the following Column; YMC-Actus Triart C18 ExRS, 30*150 mm, 5 μm; Mobile Phase A: Water (10 mmol/L NH4HCO3+0.1% NH3·H2O), Mobile Phase B: ACN; Flow rate: 60 mL/min; Gradient: 25% B to 50% B in 11 min, 50% B; Wave Length: 254/220 nm; RT1 (min): 7.25/10.2 to afford (7S)-2-(2-aminopyrimidin-4-yl)-3-[(3-chloro-2-methoxyphenyl)amino]-7-[(2S)-1,4-dioxan-2-ylmethyl]-1H,5H,6H,7H-pyrrolo[3,2-c]pyridin-4-one (57.6 mg, 19.20%) as a light yellow solid.

LC-MS: M+H found 485.1.

¹H NMR (300 MHz, DMSO-d6) δ 11.40 (s, 1H), 8.06 (d, J=5.3 Hz, J H), 7.93 (s, J H), 7.14 (s, 1H), 6.87-6.72 (m, 2H), 6.49 (d, J=5.3 Hz, 1H), 6.33 (dd, J=7.9, 1.9 Hz, 1H), 6.24 (s, 1H), 3.89 (s, 3H), 3.79 (d, J=12.2 Hz, 1H), 3.65 (q, J=10.8, 9.9 Hz, 5H), 3.48 (d, J=11.8 Hz, 2H), 3.21 (dd, J=11.2, 9.7 Hz, 2H), 3.08 (s, 1H), 1.76 (d, J=14.4 Hz, 1H), 1.59 (dt, J=14.7, 7.8 Hz, 1H).

### Example 72. afford (7R*)-7-[(2S)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (compound 530)

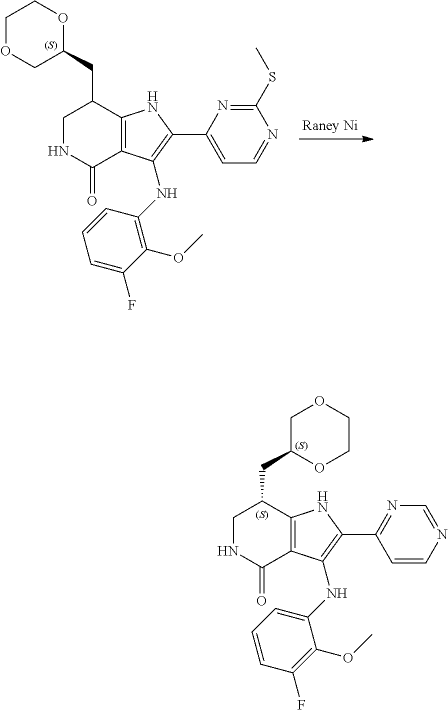

To a stirred solution of 7-[(2S)-1,4-dioxan-2-ylmethyl]-3-[(3-fluoro-2-methoxyphenyl)amino]-2-[2-(methylsulfanyl)pyrimidin-4-yl]-1H,5H,6H,7H-pyrrolo[3,2-c]pyridin-4-one (200 mg, 0.400 mmol, 1 equiv) in EtOH (2 mL) was added Raney Ni (51.45 mg, 0.600 mmol, 1.5 equiv) in portions at RT under N2 atmosphere. The resulting mixture was stirred for 2 hours at 90° C. under N2 atmosphere. The mixture was allowed to cool down to RT. The resulting mixture was filtered, then the filter cake was washed with MeOH (3*10 mL). The filtrate was concentrated under reduced pressure. The crude product was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 25 mL/min; Gradient: 24% B to 44% B in 9 min, 44% B; Wave Length: 254; 220 nm; RT1 (min): 7.58/8.72) to afford (7R*)-7-[(2S)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (47.4 mg, 26.00%) as a yellow solid.

LC-MS: (M+H)⁺ found: 454.1.

¹H NMR (300 MHz, DMSO-d6) δ 12.01 (s, 1H), 9.03 (d, J=1.4 Hz, 1H), 8.54 (d, J=5.6 Hz, 1H), 8.03 (s, 1H), 7.20 (dd, J=5.6, 1.4 Hz, 2H), 6.77 (td, J=8.3, 6.1 Hz, 1H), 6.67-6.54 (m, 1H), 6.18 (d, J=8.2 Hz, 1H), 3.95 (d, J=0.9 Hz, 3H), 3.80 (d, J=12.3 Hz, 2H), 3.66 (t, J=10.0 Hz, 3H), 3.51 (d, J=11.0 Hz, 2H), 3.20 (d, J=9.7 Hz, 3H), 1.86-1.75 (m, 1H), 1.59 (t, J=10.2 Hz, 1H).

### Example 73. (7R*)-3-[(3-chloro-2-methoxyphenyl)amino]-7-[(2S)-1,4-dioxan-2-ylmethyl]-2-(pyrimidin-4-yl)-1H,5H,6H,7H-pyrrolo[3,2-c]pyridin-4-one (compound 527)

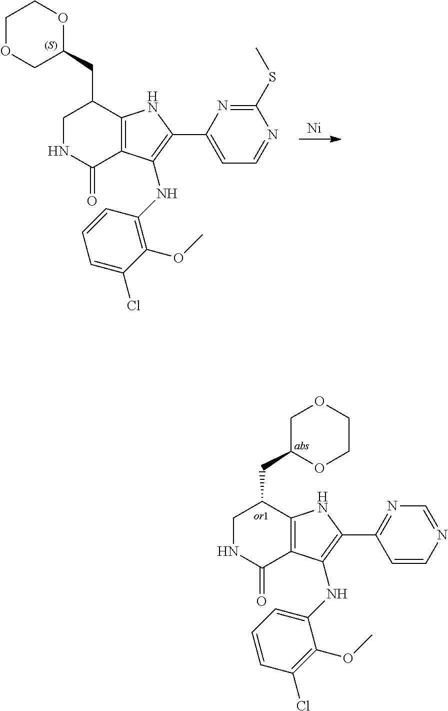

To a stirred mixture of Raney Nickel (99.62 mg, 1.162 mmol, 2 equiv) in EtOH (3 mL) was added 3-[(3-chloro-2-methoxyphenyl)amino]-7-[(2S)-1,4-dioxan-2-ylmethyl]-2-[2-(methylsulfanyl)pyrimidin-4-yl]-1H,5H,6H,7H-pyrrolo[3,2-c]pyridin-4-one (300 mg, 0.581 mmol, 1 equiv) in portions at RT under N2 atmosphere. The resulting mixture was stirred for 2 hours at 90° C. under N2 atmosphere. The resulting mixture was filtered, the filter cake was washed with MeOH. The filtrate was concentrated under reduced pressure. The crude product (200 mg) was purified by Prep-HPLC with the following conditions (Column: XSelect CSH Fluoro Phenyl, 30*150 mm, 5 μm; Mobile Phase A: Water (0.1% FA), Mobile Phase B: ACN; Flow rate: 60 mL/min; Gradient: 20% B to 40% B in 10 min, 40% B; Wave Length: 254/220 nm; RT1 (min): 7.65/8.83) to afford (7R*)-3-[(3-chloro-2-methoxyphenyl)amino]-7-[(2S)-1,4-dioxan-2-ylmethyl]-2-(pyrimidin-4-yl)-1H,5H,6H,7H-pyrrolo[3,2-c]pyridin-4-one (113.6 mg, 41.21%) as a yellow solid. LC-MS: (M+H)⁺ found: 470.4.

¹H NMR (400 MHz, DMSO-d6) δ 11.97 (s, 1H), 8.99 (d, J=1.4 Hz, 1H), 8.51 (d, J=5.6 Hz, 1H), 8.00 (s, 1H), 7.17 (dd, J=5.6, 1.5 Hz, 1H), 7.13 (s, 1H), 6.82-6.71 (m, 2H), 6.29 (dd, J=6.9, 2.7 Hz, 1H), 3.88 (s, 3H), 3.74 (ddd, J=18.8, 11.0, 2.0 Hz, 2H), 3.68-3.54 (m, 3H), 3.45 (ddd, J=12.8, 9.3, 3.6 Hz, 2H), 3.22-3.12 (m, 3H), 1.79 (ddd, J=14.3, 9.2, 5.4 Hz, 1H), 1.61-1.50 (m, 1H).

### Example 74. (7S)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (35.3 mg, 22.83%) (7S)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (compound 525)

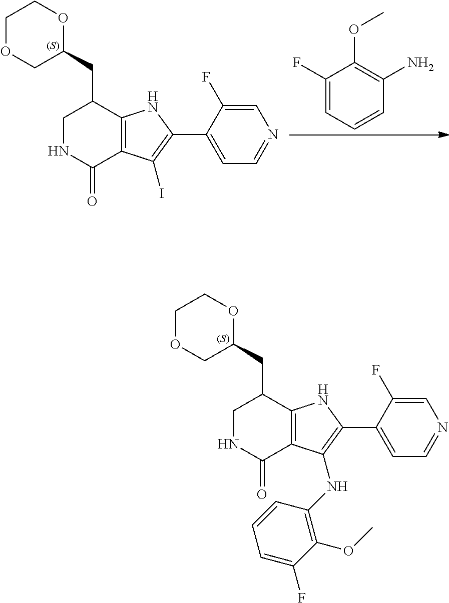

To a stirred solution of 7-(1,4-dioxan-2-ylmethyl)-2-(3-fluoropyridin-4-yl)-3-iodo-1H,5H,6H,7H-pyrrolo[3,2-c]pyridin-4-one (600 mg, 1.312 mmol, 1.00 equiv) and 3-fluoro-2-methoxyaniline (555.64 mg, 3.936 mmol, 3.00 equiv) in DMF (7 mL) were added Cs₂CO₃ (855.09 mg, 2.624 mmol, 2.0 equiv) and EPhos Pd G4 (241.07 mg, 0.262 mmol, 0.2 equiv) and EPhos (140.35 mg, 0.262 mmol, 0.20 equiv) at rt, then the solution was stirred at 50° C. under N2 atmosphere about 3 h. The resulting mixture was extracted with EA (3×20 mL). The combined organic layers were washed with saturated salt solution (3×20 mL), dried over anhydrous sodium sulfate. After filtration, the filtrate was concentrated under reduced pressure. The residue was purified by Prep-TLC (DCM: MeOH=20.1) to afford crude product 7-(1,4-dioxan-2-ylmethyl)-3-[(3-fluoro-2-methoxyphenyl)amino]-2-(3-fluoropyridin-4-yl)-1H,5H,6H,7H-pyrrolo[3,2-c]pyridin-4-one (160 mg, 25.92%) as a yellow solid.

LC-MS: M+H found: 471.0.

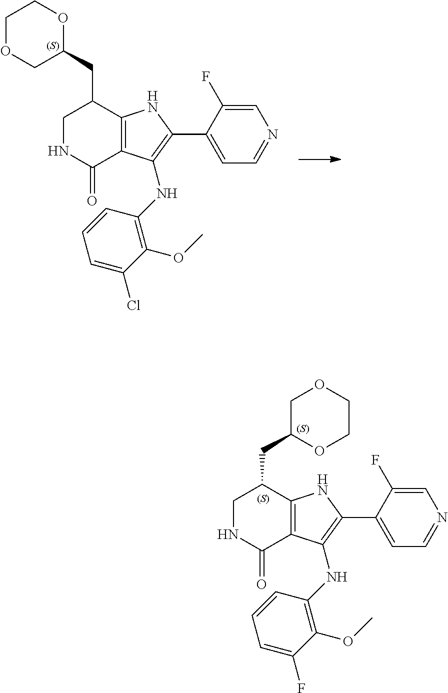

The crude product 3-[(3-chloro-2-methoxyphenyl)amino]-7-[(2S)-1,4-dioxan-2-ylmethyl]-2-(3-fluoropyridin-4-yl)-1H,5H,6H,7H-pyrrolo[3,2-c]pyridin-4-one (160 mg, 0.329 mmol, 1.00 equiv) was purified by Prep-HPLC with the following conditions (Column: X Bridge Prep Phenyl OBD Column, 19 250 mm, 5 μm; Mobile Phase A: Water (10 mmol/L NH4HCO3+0.1% NH3 H2O), Mobile Phase B: MeOH-HPLC; Flow rate: 25 mL/min; Gradient: 60% B to 74% B in 10 min, 74% B; Wave Length: 254; 220 nm) to afford (7S)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (35.3 mg, 22.83%) (7S)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (35.3 mg, 22.83%) as a light yellow solid.

LC-MS: M+H found: 471.0.

¹H NMR (300 MHz, DMSO-d6) δ 11.39 (s, 1H), 8.50 (d, J=3.0 Hz, 1H), 8.28 (d, J=5.2 Hz, 1H), 7.53 (s, 1H), 7.46-7.40 (m, 1H), 7.16 (s, 1H), 6.65-6.55 (m, 1H), 6.52-6.44 (m, 1H), 5.98 (d, J=8.2 Hz, 1H), 3.88 (s, 3H), 3.81-3.73 (m, 1H), 3.69-3.58 (m, 4H), 3.56-3.40 (m, 2H), 3.17 (dd, J=25.1, 14.8 Hz, 3H), 1.81-1.70 (m, 1H), 1.66-1.53 (m, 1H).

### Example 75. (7S)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (compound 551)

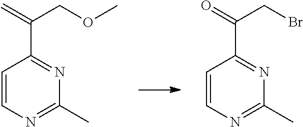

To a stirred solution of 4-(1-ethoxyethenyl)-2-methylpyrimidine (250 mg, 1.522 mmol, 1.00 equiv) in THF (3 mL, 30.851 mmol, 20.27 equiv) was added NBS (325.17 mg, 1.826 mmol, 1.2 equiv) at rt under N2 atmosphere. Then, the solution was stirred at rt for 2 h. The resulting mixture was diluted with water (100 ml) and extracted with EA (3×100 ml). The extracts was concentrated under reduced pressure to afford 2-bromo-1-(2-methylpyrimidin-4-yl)ethanone (283 mg, 86.44%) as a yellow oil.

LC-MS: M−18 found: 232.95.

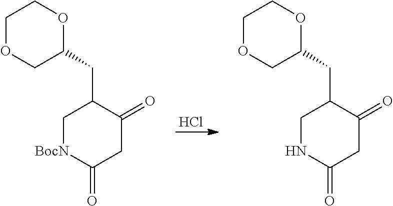

To a stirred solution of tert-butyl 5-(1,4-dioxan-2-ylmethyl)-2,4-dioxopiperidine-1-carboxylate (690 mg, 2.202 mmol, 1.00 equiv) in DCM (14.79 mL, 232.575 mmol, 105.62 equiv) was added HCl (gas) in 1,4-dioxane (7.89 mL, 259.528 mmol, 117.86 equiv) at rt under N2 atmosphere. Then, the solution was stirred at rt for 2 h. The resulting mixture was concentrated under reduced pressure to afford crude 5-(1,4-dioxan-2-ylmethyl)piperidine-2,4-dione (470 mg, 100.10%) as a yellow oil.

LC-MS: M+1 found: 214.25.

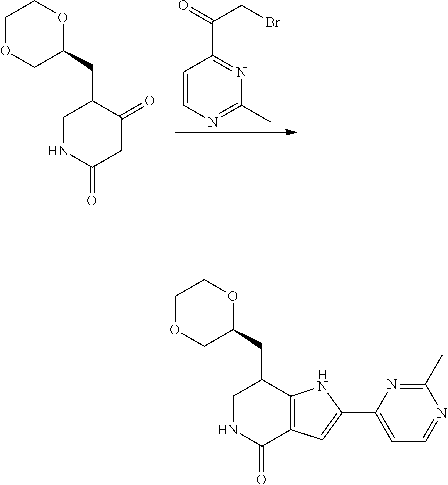

To a stirred solution of 5-[(2R)-1,4-dioxan-2-ylmethyl]piperidine-2,4-dione (272 mg, 1.276 mmol, 1.00 equiv) and NH₄OAc (589.96 mg, 7.656 mmol, 6.0 equiv) in EtOH (5 mL) were added 2-bromo-1-(2-methylpyrimidin-4-yl)ethanone (274.32 mg, 1.276 mmol, 1.0 equiv) at RT under N2 atmosphere. Then, the solution was stirred at 50° C. for 16 h. The resulting mixture was concentrated under reduced pressure and purified by silica gel column chromatography, eluted with DCM:MeOH=7% to afford 7-(1,4-dioxan-2-ylmethyl)-2-(2-methylpyrimidin-4-yl)-1H,5H,6H,7H-pyrrolo[3,2-c]pyridin-4-one (140 mg, 33.42%) as a yellow oil.

LC-MS: M+H found: 329.0.

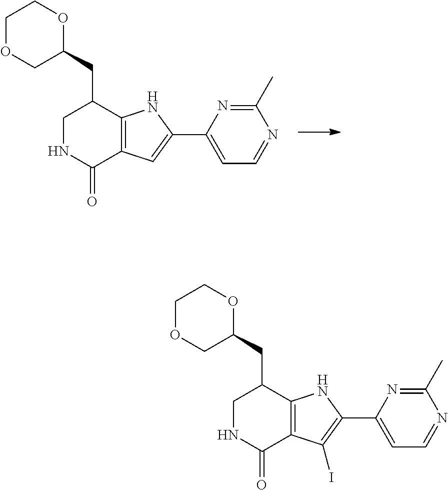 [SMILES: Cc1nccc(-c2cc3c([nH]2)C(C[C@H]2COCCO2)CNC3=O)n1 ; Cc1nccc(-c2[nH]c3c(c2I)C(=O)NCC3C[C@H]2COCCO2)n1]

To a stirred solution of 7-(1,4-dioxan-2-ylmethyl)-2-(2-methylpyrimidin-4-yl)-1H,5H,6H,7H-pyrrolo[3,2-c]pyridin-4-one (140 mg, 0.426 mmol, 1.00 equiv) in DMF (3 mg) were added NIS (124.70 mg, 0.554 mmol, 1.3 equiv) at rt under N2 atmosphere. Then, the solution was stirred at rt for 2 h. The resulting mixture was concentrated under reduced pressure and purified by Prep-TLC (DCM:MeOH=15:1) to afford 7-(1,4-dioxan-2-ylmethyl)-3-iodo-2-(2-methylpyrimidin-4-yl)-1H,5H,6H,7H-pyrrolo[3,2-c]pyridin-4-one (130 mg, 67.12%) as a yellow solid.

LC-MS: M+H found: 454.85.

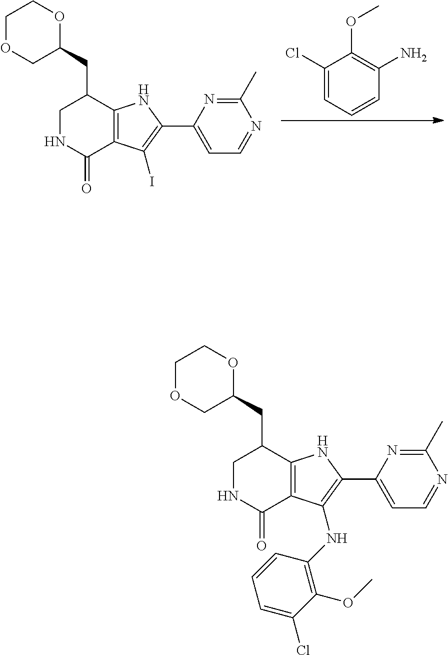

To a stirred solution of 7-[(2S)-1,4-dioxan-2-ylmethyl]-3-iodo-2-(2-methylpyrimidin-4-yl)-1H,5H,6H,7H-pyrrolo[3,2-c]pyridin-4-one (130 mg, 0.286 mmol, 1 equiv) and Cs2CO3 (186.48 mg, 0.572 mmol, 2.0 equiv) in DMF (1.5 mL) were added 3-chloro-2-methoxyaniline (180.40 mg, 1.144 mmol, 4.0 equiv) and EPhos Pd G4 (52.57 mg, 0.057 mmol, 0.2 equiv) at RT under N2 atmosphere. The solution was stirred at 50° C. for 16 h. The resulting mixture was concentrated under reduced pressure and purified by Prep-TLC (DCM:MeOH=15:1) to afford 3-[(3-chloro-2-methoxyphenyl)amino]-7-[(2S)-1,4-dioxan-2-ylmethyl]-2-(2-methylpyrimidin-4-yl)-1H,5H,6H,7H-pyrrolo[3,2-c]pyridin-4-one (70 mg, 50.54%) as a yellow solid.

LC-MS: M+H found: 484.0.

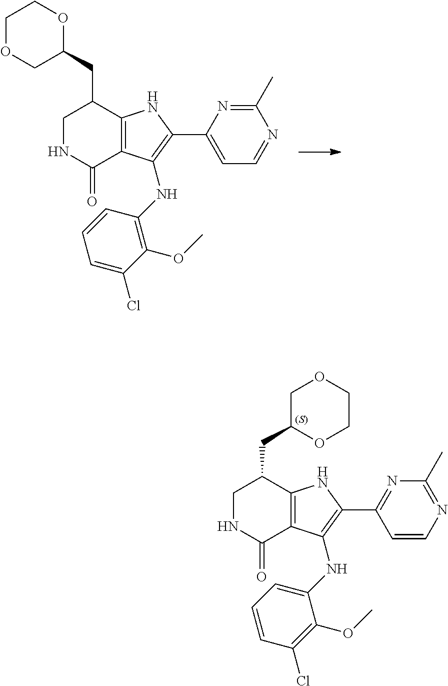

The crude product 3-[(3-chloro-2-methoxyphenyl)amino]-7-[(2S)-1,4-dioxan-2-ylmethyl]-2-(2-methylpyrimidin-4-yl)-1H,5H,6H,7H-pyrrolo[3,2-c]pyridin-4-one (70 mg, 0.145 mmol, 1 equiv) was purified by Prep-HPLC with the following conditions (Column: XBridge Prep Phenyl OBD Column, 19*250 mm, 5 μm; Mobile Phase A: Water (10 mmol/L NH4HCO3+0.1% NH3·H2O), Mobile Phase B: MeOH-HPLC; Flow rate: 25 mL/min; Gradient: 68% B to 78% B in 8 min, 78% B; Wave Length: 254; 220 nm; RT1 (min): 6.42, 7.18) to afford (7S)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (14.2 mg, 20.29%) as a light yellow solid.

LC-MS: M found 484.0.

¹H NMR (300 MHz, DMSO-d6) δ 11.74 (s, 1H), 8.45 (d, J=5.5 Hz, 1H), 8.21 (s, 1H), 7.19 (s, 1H), 7.07 (d, J=5.6 Hz, 1H), 6.87-6.74 (m, 2H), 6.33 (dd, J=6.8, 2.7 Hz, 1H), 3.90 (s, 3H), 3.82-3.70 (m, 2H), 3.66 (d, J=11.1 Hz, 4H), 3.49 (dd, J=12.5, 3.8 Hz, 2H), 3.19 (d, J=10.0 Hz, 1H), 3.09 (s, 1H), 2.60 (s, 3H), 1.75 (d, J=13.8 Hz, 1H), 1.65 (t, J=7.8 Hz, 1H).

### Example 76. (7R)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (compound 575)

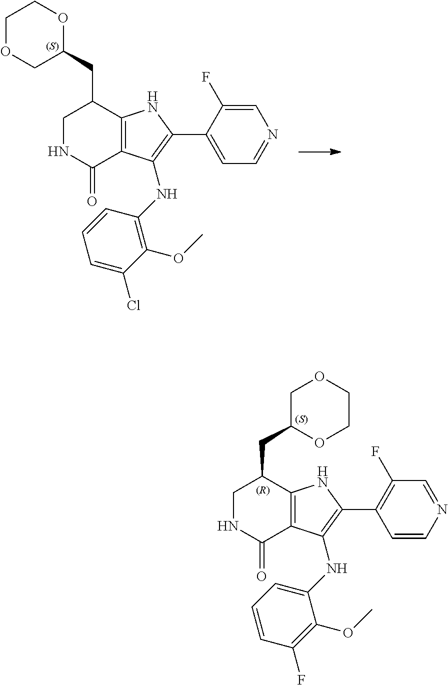

The crude product 3-[(3-chloro-2-methoxyphenyl)amino]-7-[(2S)-1,4-dioxan-2-ylmethyl]-2-(3-fluoropyridin-4-yl)-1H,5H,6H,7H-pyrrolo[3,2-c]pyridin-4-one (160 mg, 0.329 mmol, 1.00 equiv) was purified by Prep-HPLC with the following conditions (Column: XBridge Prep Phenyl OBD Column, 19*250 mm, 5 μm; Mobile Phase A. Water (10 mmol/L NH4HCO3+0.1% NH3·H2O), Mobile Phase B: MeOH-HPLC; Flow rate: 25 mL/min; Gradient: 60% B to 74% B in 10 min, 74% B; Wave Length: 254; 220 nm; RT1 (min): 7.18, 8.9) to afford (7R)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (26.6 mg, 17.21%) as a light yellow solid.

LC-MS: M+H found: 471.0.

¹H NMR (300 MHz, DMSO-d6) δ 11.49 (s, 11H), 8.51 (d, J=3.0 Hz, 1H), 8.31-8.26 (m, 1H), 7.57 (s, 1H), 7.44 (dd, J=6.9, 5.1 Hz, 1H), 7.17 (s, 1H), 6.68-6.59 (m, 1H), 6.56-6.46 (m, 1H), 6.00 (d, J=8.2 Hz, 1H), 3.92-3.89 (m, 3H), 3.83-3.66 (m, 5H), 3.51 (t, J=10.0 Hz, 2H), 3.26-3.17 (m, 3H), 1.80 (dd, J=14.8, 9.6 Hz, 1H), 1.68-1.54 (m, 1H).

### Example 77. (7R*)-3-[(3-chloro-2-methoxyphenyl)amino]-7-[(2S)-1,4-dioxan-2-ylmethyl]-2-(pyrimidin-4-yl)-1H,5H,6H,7H-pyrrolo[3,2-c]pyridin-4-one (compound 574)

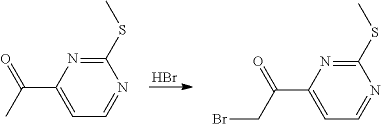

To a stirred solution of 1-[2-(methylsulfanyl)pyrimidin-4-yl]ethanone (3 g, 17.835 mmol, 1 equiv) in HBr in AcOH (40%) (3 mL, 102.704 mmol, 5.76 equiv) and HAc (10 mL) was added Br2 (1.13 mL, 22.054 mmol, 1.24 equiv) dropwise at 0° C. under N2 atmosphere. The resulting mixture was stirred for 2.5 hours at 80° C. under N2 atmosphere. The mixture was allowed to cool down to RT. The resulting mixture was re-crystallized from EA 200 mL. The precipitated solids were collected by filtration to afford 2-bromo-1-[2-(methylsulfanyl)pyrimidin-4-yl]ethanone (4 g, 90.76%) as a yellow solid.

LC-MS: M+H found: 246.95.

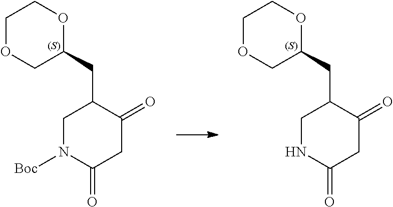

To a stirred solution of tert-butyl 5-[(2S)-1,4-dioxan-2-ylmethyl]-2,4-dioxopiperidine-1-carboxylate (4 g, 12.765 mmol, 1 equiv) in DCM (40 mL) was added TFA (20 mL, 269.261 mmol, 21.09 equiv) dropwise at RT. The resulting mixture was stirred for 1.5 hour at RT. The aqueous layer was extracted with DCM and H2O (3×1 150 mL). To afford 5-[(2S)-1,4-dioxan-2-ylmethyl]piperidine-2,4-dione (2.6 g, 95.52%) as a light yellow solid.

LC-MS: M−H found: 212.05.

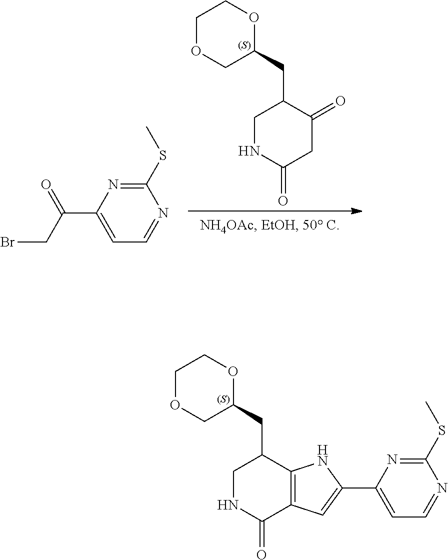

To a stirred solution of 2-bromo-1-[2-(methylsulfanyl)pyrimidin-4-yl]ethanone (2.4 g, 9.712 mmol, 1 equiv) and 5-[(2S)-1,4-dioxan-2-ylmethyl]piperidine-2,4-dione (2.07 g, 9.712 mmol, 1 equiv) in EtOH (20 mL) was added NH₄OAc (3.74 g, 48.560 mmol, 5 equiv) in portions at RT. The resulting mixture was stirred for 20 hours at 50° C. under N2 atmosphere. The mixture was allowed to cool down to RT. The resulting mixture was extracted with EA and Water (3×150 mL). The combined organic dried over anhydrous Na2SO4. After filtration, the filtrate was concentrated under reduced pressure. The residue was purified by silica gel column chromatography, eluted with DCM and MeOH (95:5) to afford 7-[(2S)-1,4-dioxan-2-ylmethyl]-2-[2-(methylsulfanyl)pyrimidin-4-yl]-1H,5H,6H,7H-pyrrolo[3,2-c]pyridin-4-one (1.15 g, 32.85%) as a yellow solid.

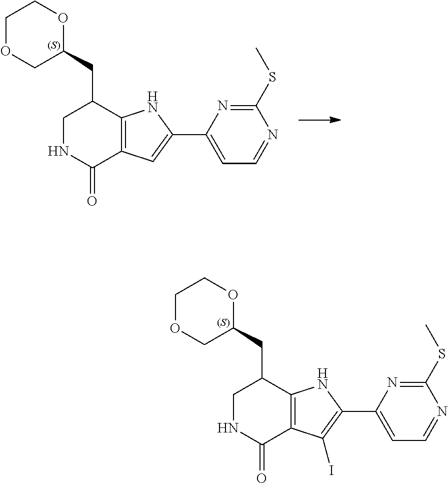

To a stirred solution of 7-[(2S)-1,4-dioxan-2-ylmethyl]-2-[2-(methylsulfanyl)pyrimidin-4-yl]-1H,5H,6H,7H-pyrrolo[3,2-c]pyridin-4-one (1.4 g, 3.884 mmol, 1 equiv) in DMF (20 mL) was added NIS (1.31 g, 5.826 mmol, 1.5 equiv) in portions at RT. The resulting mixture was stirred for 1 hour at RT. The resulting mixture was extracted with EA (3×50 mL). The combined organic layer was concentrated under reduced pressure. The residue was purified by Prep-TLC (DCM and MeOH 15:1) to afford 7-[(2S)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.65 g, 87.35%) as a yellow solid.

LC-MS: M−H found: 487.0.

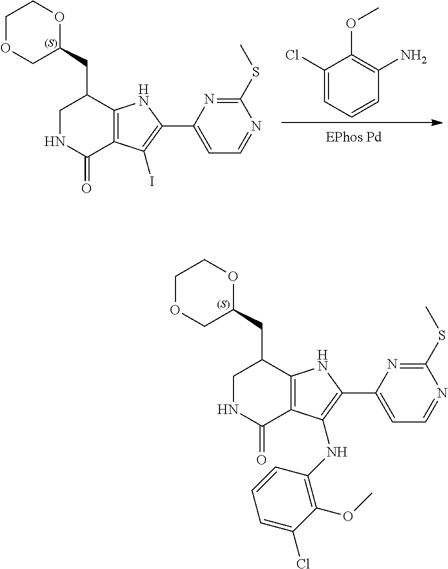

To a stirred mixture of 7-[(2S)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1 g, 2.056 mmol, 1 equiv) and 3-chloro-2-methoxyaniline (0.97 g, 6.168 mmol, 3 equiv) in DMF (10 mL) were added Cs₂CO₃ (2.01 g, 6.168 mmol, 3 equiv) and EPhos Pd G4 (0.19 g, 0.206 mmol, 0.1 equiv) in portions at RT under N2 atmosphere. The resulting mixture was stirred for 2 hours at 50 degrees C. under N2 atmosphere. The resulting mixture was filtered, then the filter cake was washed with EA (1×1 100 mL). The filtrate was concentrated under reduced pressure. The residue was purified by Prep-TLC (DCM and MeOH 20:1) to afford 3-[(3-chloro-2-methoxyphenyl)amino]-7-[(2S)-1,4-dioxan-2-ylmethyl]-2-[2-(methylsulfanyl)pyrimidin-4-yl]-1H,5H,6H,7H-pyrrolo[3,2-c]pyridin-4-one (1.04 g, 98.02%) as a light yellow solid.

LC-MS: M+H found: 516.1.

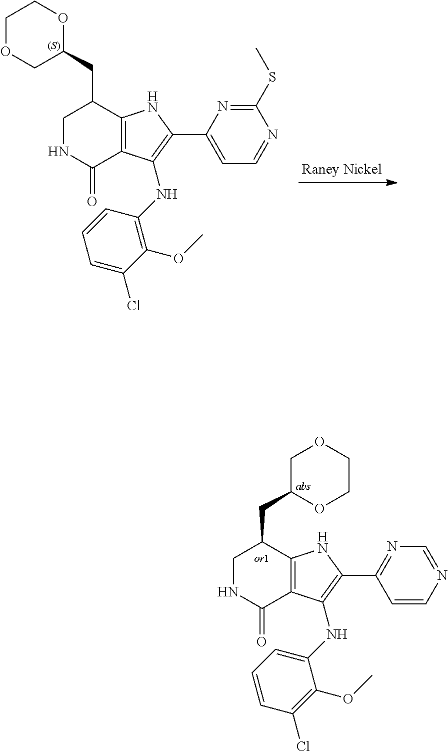

To a stirred mixture of Raney Nickel (99.62 mg, 1.162 mmol, 2 equiv) in EtOH (3 mL) was added 3-[(3-chloro-2-methoxyphenyl)amino]-7-[(2S)-1,4-dioxan-2-ylmethyl]-2-[2-(methylsulfanyl)pyrimidin-4-yl]-1H,5H,6H,7H-pyrrolo[3,2-c]pyridin-4-one (300 mg, 0.581 mmol, 1 equiv) in portions at RT under N2 atmosphere. The resulting mixture was stirred for 2 hours at 90° C. under N2 atmosphere. The resulting mixture was filtered, then the filter cake was washed with MeOH. The filtrate was concentrated under reduced pressure. The crude product (200 mg) was purified by Prep-HPLC with the following conditions (Column: XSelect CSH Fluoro Phenyl, 30*150 mm, 5 μm; Mobile Phase A: Water (0.1% FA), Mobile Phase B. ACN; Flow rate: 60 mL/min; Gradient: 20% B to 40% B in 10 min, 40% B; Wave Length: 254/220 nm; RT1 (min): 7.65/8.83) to afford (7R*)-3-[(3-chloro-2-methoxyphenyl)amino]-7-[(2S)-1,4-dioxan-2-ylmethyl]-2-(pyrimidin-4-yl)-1H,5H,6H,7H-pyrrolo[3,2-c]pyridin-4-one (109.8 mg, 39.34%) as a yellow solid.

LC-MS: (M+H)⁺ found 470.4.

¹H NMR (400 MHz, DMSO-d6) δ 11.97 (s, 1H), 8.99 (d, J=1.4 Hz, 1H), 8.51 (d, J=5.6 Hz, 1H), 8.00 (s, 1H), 7.17 (dd, J=5.6, 1.5 Hz, 1H), 7.13 (s, 1H), 6.82-6.71 (m, 2H), 6.29 (dd, J=6.9, 2.7 Hz, 1H), 3.88 (s, 3H), 3.74 (ddd, J=18.8, 11.0, 2.0 Hz, 2H), 3.68-3.54 (m, 3H), 3.45 (ddd, J=12.8, 9.3, 3.6 Hz, 2H), 3.22-3.12 (m, 3H), 1.79 (ddd, J=14.3, 9.2, 5.4 Hz, 1H), 1.61-1.50 (m, 1H).

### Example 78 (7R)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (compound 573)

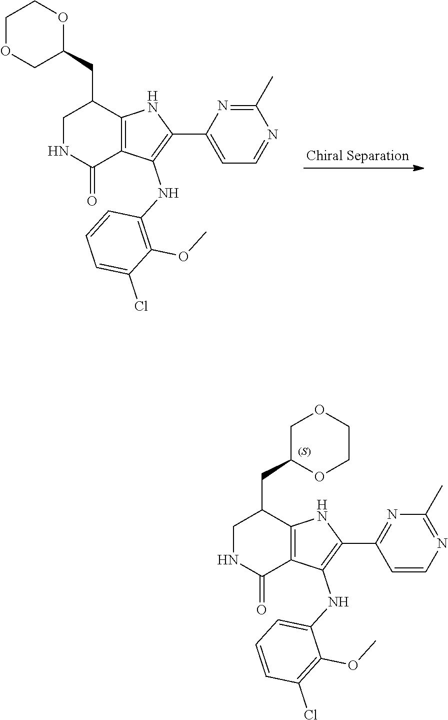

The crude product 3-[(3-chloro-2-methoxyphenyl)amino]-7-[(2S)-1,4-dioxan-2-ylmethyl]-2-(2-methylpyrimidin-4-yl)-1H,5H,6H,7H-pyrrolo[3,2-c]pyridin-4-one (70 mg, 0.145 mmol, 1 equiv) was purified by Prep-HPLC with the following conditions (Column: XBridge Prep Phenyl OBD Column, 19*250 mm, 5 μm; Mobile Phase A: Water (10 mmol/L NH4HCO3+0. 1% NH3·H2O), Mobile Phase B: MeOH-HPLC; Flow rate: 25 mL/min; Gradient: 68% B to 78% B in 8 min, 78% B; Wave Length: 254; 220 nm; RT1 (min): 6.42, 7.18) to afford (7R)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (17.3 mg, 24.71%) as a light yellow solid.

LC-MS: M+H found: 484.0.

¹H NMR (300 MHz, DMSO-d6) δ 11.83 (s, 1H), 8.45 (d, J=5.5 Hz, 1H), 8.27 (s, 1H), 7.18 (s, 1H), 7.06 (d, J=5.5 Hz, 1H), 6.87-6.74 (m, 2H), 6.36 (dd, J=6.8, 2.8 Hz, 1H), 3.90 (s, 3H), 3.84 (d, J=11.7 Hz, 2H), 3.70 (q, J=11.7 Hz, 4H), 3.53 (d, J=11.3 Hz, 1H), 3.46 (s, 1H), 3.22 (d, J=6.1 Hz, 2H), 2.58 (s, 3H), 1.77 (s, 1H), 1.59 (s, 1H).

### Example 79. (7R*)-7-[(2S)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (compound 572)

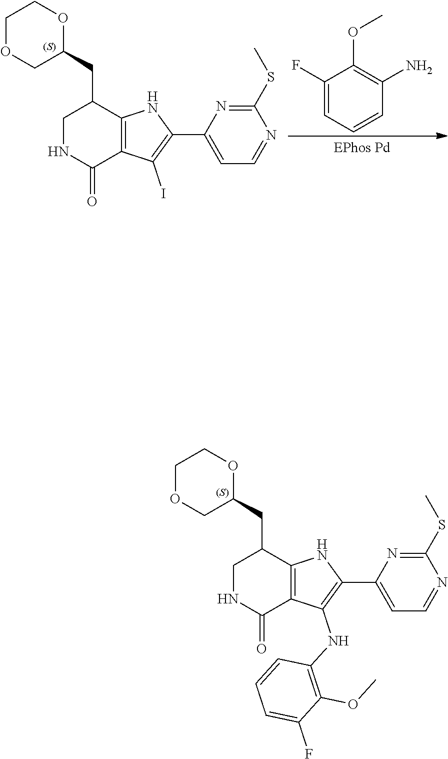

To a stirred mixture of 7-[(2S)-1,4-dioxan-2-ylmethyl]-3-iodo-2-[2-(methylsulfanyl)pyrimidin-4-yl]-1H,5H,6H,7H-pyrrolo[3,2-c]pyridin-4-one (400 mg, 0.822 mmol, 1 equiv) and 3-fluoro-2-methoxyaniline (348.27 mg, 2.466 mmol, 3 equiv) in DMF (5 mL) were added Cs₂CO₃ (803.95 mg, 2.466 mmol, 3 equiv) and EPhos Pd G4 (75.55 mg, 0.082 mmol, 0.1 equiv) in portions at RT under N2 atmosphere. The resulting mixture was stirred for 2 hours at 50 degrees C. under N2 atmosphere. The resulting mixture was filtered, then the filter cake was washed with EA (1×1 30 mL). The filtrate was concentrated under reduced pressure. The residue was purified by Prep-TLC (DCM and MeOH 20:1) to afford 7-[(2S)-1,4-dioxan-2-ylmethyl]-3-[(3-fluoro-2-methoxyphenyl)amino]-2-[2-(methylsulfanyl)pyrimidin-4-yl]-1H,5H,6H,7H-pyrrolo[3,2-c]pyridin-4-one (363 mg, 88.35%) as a light yellow solid.

LC-MS: M+H found: 500.45.

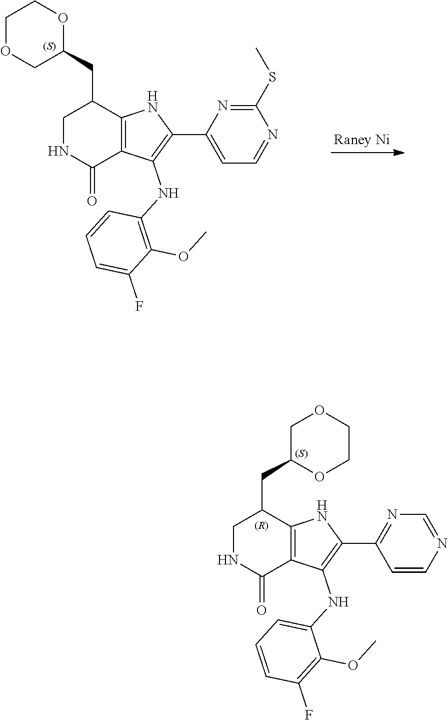

To a stirred solution of 7-[(2S)-1,4-dioxan-2-ylmethyl]-3-[(3-fluoro-2-methoxyphenyl)amino]-2-[2-(methylsulfanyl)pyrimidin-4-yl]-1H,5H,6H,7H-pyrrolo[3,2-c]pyridin-4-one (200 mg, 0.400 mmol, 1 equiv) in EtOH (2 mL) was added Raney Ni (51.45 mg, 0.600 mmol, 1.5 equiv) in portions at RT under N2 atmosphere. The resulting mixture was stirred for 2 hours at 90° C. under N2 atmosphere. The mixture was allowed to cool down to RT. The resulting mixture was filtered, then the filter cake was washed with MeOH (3*10 mL). The filtrate was concentrated under reduced pressure. The crude product was purified by Prep-HPLC with the following conditions (Column: XSelect CSH Prep C18 OBD Column, 19*250 mm, 5 μm; Mobile Phase A: Water (0.1% FA), Mobile Phase B: ACN; Flow rate: 25 mL/min; Gradient: 24% B to 44% B in 9 min, 44% B; Wave Length: 254; 220 nm; RT1 (min): 7.58/8.72) to afford (7R*)-7-[(2S)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (42.9 mg, 23.37%) as a yellow solid.

LC-MS: (M+H)⁺ found 454.1.

¹H NMR (300 MHz, DMSO-d6) δ 12.01 (s, 1H), 9.03 (d, J=1.4 Hz, 1H), 8.54 (d, J=5.6 Hz, 1H), 8.03 (s, 1H), 7.20 (dd, J=5.6, 1.4 Hz, 2H), 6.77 (td, J=8.3, 6.1 Hz, 1H), 6.67-6.54 (m, 1H), 6.18 (d, J=8.2 Hz, 1H), 3.95 (d, J=0.9 Hz, 3H), 3.80 (d, J=12.3 Hz, 2H), 3.66 (t, J=10.0 Hz, 3H), 3.51 (d, J=11.0 Hz, 2H), 3.20 (d, J=9.7 Hz, 3H), 1.86-1.75 (m, 1H), 1.59 (t, J=10.2 Hz, 1H).

### Example 80. (7R)-2-(2-aminopyrimidin-4-yl)-3-[(3-chloro-2-methoxyphenyl)amino]-7-[(2S)-1,4-dioxan-2-ylmethyl]-1H,5H,6H,7H-pyrrolo[3,2-c]pyridin-4-one (compound 571)

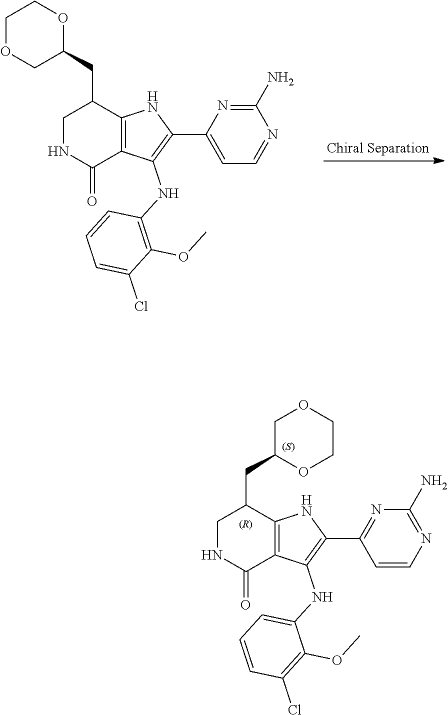

The crude product 2-(2-aminopyrimidin-4-yl)-3-[(3-chloro-2-methoxyphenyl)amino]-7-[(2S)-1,4-dioxan-2-ylmethyl]-1H,5H,6H,7H-pyrrolo[3,2-c]pyridin-4-one (300 mg, 0.619 mmol, 1 equiv) was purified by Prep-HPLC as the following Column: YMC-Actus Triart C18 ExRS, 30*150 mm, 5 μm; Mobile Phase A: Water (10 mmol/L NH4HCO3+0.1% NH3·H2O), Mobile Phase B: ACN; Flow rate: 60 mL/min; Gradient: 25% B to 50% B in 11 min, 50% B; Wave Length: 254/220 nm; RT1 (min): 7.25/10.2; to afford (7R)-2-(2-aminopyrimidin-4-yl)-3-[(3-chloro-2-methoxyphenyl)amino]-7-[(2S)-1,4-dioxan-2-ylmethyl]-1H,5H,6H,7H-pyrrolo[3,2-c]pyridin-4-one (54.0 mg, 18.00%) as a light yellow solid.

LC-MS: M found 485.1.

¹H NMR (300 MHz, DMSO-d6) δ 11.52 (s, 1H), 8.06 (d, J=5.3 Hz, 1H), 7.96 (s, 1H), 7.15 (s, 1H), 6.87-6.69 (m, 2H), 6.47 (d, J=5.3 Hz, 1H), 6.35 (dd, J=7.8, 1.9 Hz, 1H), 6.24 (s, 2H), 3.90 (s, 4H), 3.78-3.38 (m, 6H), 3.20 (d, J=13.5 Hz, 3H), 1.72 (d, J=10.6 Hz, 1H), 1.56 (t, J=7.8 Hz, 1H).

### Example 81. (7R)-7-[(2S)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (compound 606)

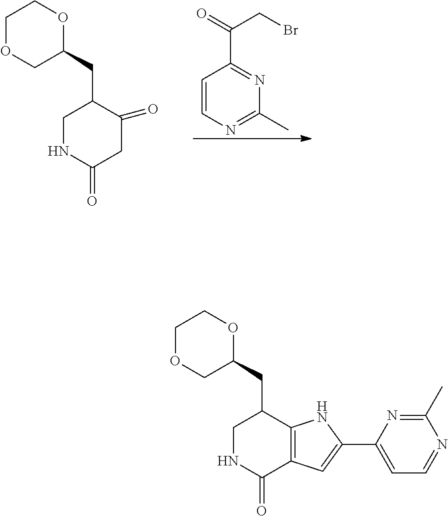 [SMILES: O=C1CC(=O)C(C[C@H]2COCCO2)CN1 ; Cc1nccc(C(=O)CBr)n1 ; Cc1nccc(-c2cc3c([nH]2)C(C[C@H]2COCCO2)CNC3=O)n1]

To a stirred solution of 5-[(2S)-1,4-dioxan-2-ylmethyl]piperidine-2,4-dione (1 g, 4.690 mmol, 1 equiv) and 2-bromo-1-(2-methylpyrimidin-4-yl)ethanone (1.21 g, 5.628 mmol, 1.2 equiv) in EtOH (10 mL) was added NH₄OAc (1.81 g, 23.450 mmol, 5 equiv) at rt, then the solution was stirred at 50° C. about 16 h. The resulting mixture was concentrated under reduced pressure. The residue was purified by silica gel column chromatography, eluted with MeOH:DCM=15:1 to afford 7-[(2S)-1,4-dioxan-2-ylmethyl]-2-(2-methylpyrimidin-4-yl)-1H,5H,6H,7H-pyrrolo[3,2-c]pyridin-4-one (400 mg, 25.97%) as a yellow solid.

LC-MS: M+H found: 329.2.

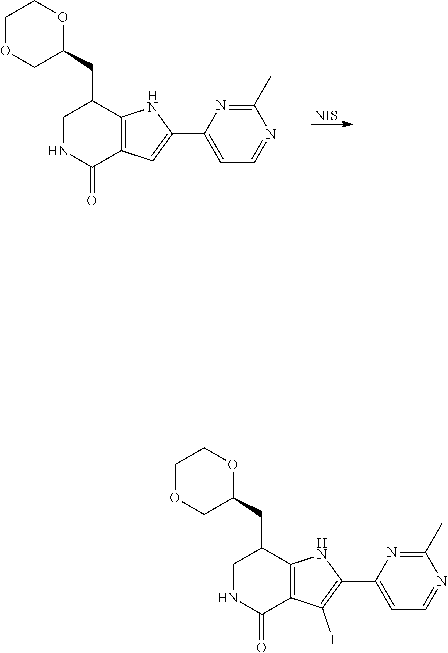

To a stirred solution of 7-[(2S)-1,4-dioxan-2-ylmethyl]-2-(2-methylpyrimidin-4-yl)-1H,5H,6H,7H-pyrrolo[3,2-c]pyridin-4-one (400 mg, 1.218 mmol, 1 equiv) in DMF (10 mL) was added NIS (356.28 mg, 1.583 mmol, 1.3 equiv)) at rt, Then the solution was stirred at rt about 2 h. The resulting mixture was concentrated under reduced pressure. The residue was purified by column chromatography with the following conditions (MeOH:DCM=15:1) to afford 7-[(2S)-1,4-dioxan-2-ylmethyl]-3-iodo-2-(2-methylpyrimidin-4-yl)-1H,5H,6H,7H-pyrrolo[3,2-c]pyridin-4-one (400 mg, 72.29%) as a yellow solid.

LC-MS: M+H found: 455.0.

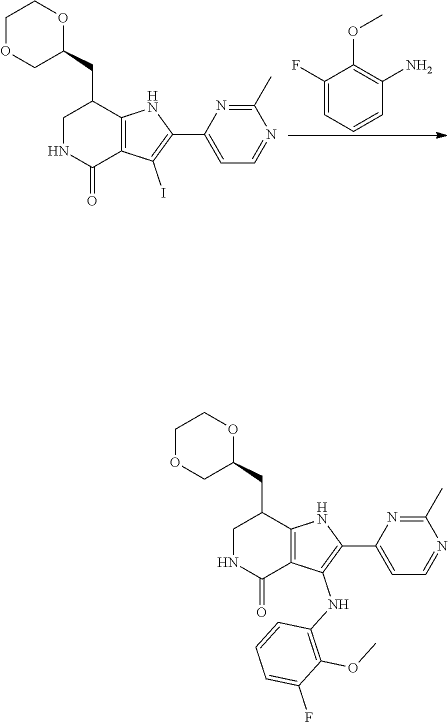

To a stirred solution of 7-[(2S)-1,4-dioxan-2-ylmethyl]-3-iodo-2-(2-methylpyrimidin-4-yl)-1H,5H,6H,7H-pyrrolo[3,2-c]pyridin-4-one (400 mg, 0.881 mmol, 1 equiv) and 3-fluoro-2-methoxyaniline (149.14 mg, 1.057 mmol, 1.2 equiv) in DMF (4 mL) were added EPhos Pd G4 (80.88 mg, 0.088 mmol, 0.1 equiv) and EPhos (94.18 mg, 0.176 mmol, 0.2 equiv) at rt. Then the solution was stirred 50° C. under N2 atmosphere for 2 h. The resulting mixture was extracted with EA (3×20 mL). The combined organic layers were washed with saturated salt solution (3×20 mL), dried over anhydrous sodium sulfate. After filtration, the filtrate was concentrated under reduced pressure. The residue was purified by Prep-TLC (DCM:MeOH=20:1) to afford 7-[(2S)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (350 mg, 85.02%) as a yellow solid.

LC-MS: M+H found: 468.4.

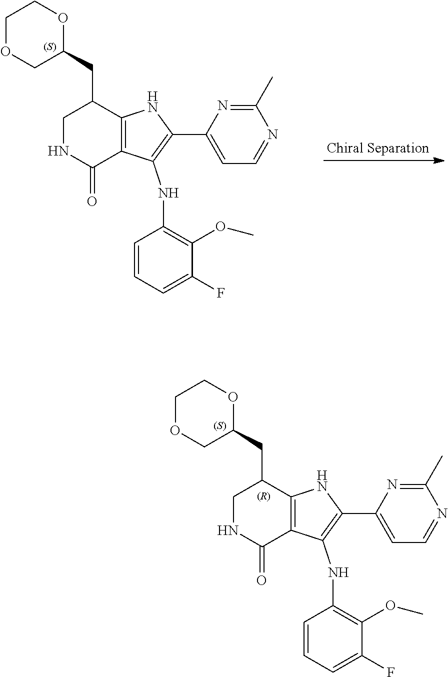

The residue 7-[(2S)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (400 mg, 0.856 mmol, 1 equiv) was purified by reverse flash chromatography with the following conditions: Column: XBridge Prep OBD C18 Column, 30*150 mm, 5 μm; Mobile Phase A: Water (10 mmol/L NH4HCO3+0.1% NH3·H2O), Mobile Phase B: MeOH-HPLC; Flow rate: 60 mL/min; Gradient: 65% B to 77% B in 10 min, 77% B; Wave Length: 254; 220 nm; RT1 (min): 8.4, 9.43 (min), to afford (7R)-7-[(2S)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (71.1 mg, 17.74%) as a yellow solid.

LC-MS: M+H found: 468.1.

¹H NMR (400 MHz, DMSO-d6) δ 11.72 (s, 1H), 8.45 (d, J=5.5 Hz, 1H), 8.14 (s, 1H), 7.19 (s, 1H), 7.06 (d, J=5.5 Hz, 1H), 6.76 (td, J=8.2, 5.9 Hz, 1H), 6.59 (dd, J=10.7, 8.5 Hz, 1H), 6.18 (d, J=8.3 Hz, 1H), 3.94 (s, 3H), 3.81-3.69 (m, 2H), 3.69-3.63 (m, 3H), 3.63-3.50 (m, 2H), 3.50-3.42 (m, 1H), 3.21 (t, J=10.6 Hz, 1H), 3.14-3.07 (m, 1H), 2.60 (s, 3H), 1.75 (dt, J=14.2, 4.7 Hz, 1H), 1.62 (dt, J=14.7, 8.0 Hz, 1H).

### Example 82. (7S)-7-[(2S)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (compound 603)

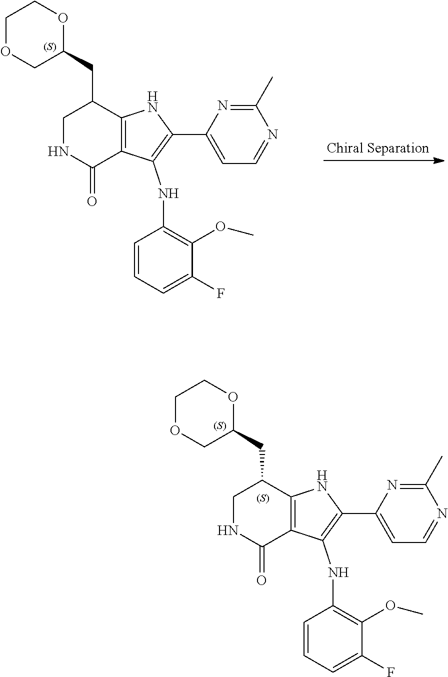

The residue 7-[(2S)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (400 mg, 0.856 mmol, 1 equiv) was purified by reverse flash chromatography with the following conditions: Column: XBridge Prep OBD C18 Column, 30*150 mm, 5 μm; Mobile Phase A: Water (10 mmol/L NH4HCO3+0.1% NH3·H2O), Mobile Phase B: MeOH-HPLC; Flow rate: 60 mL/min: Gradient: 65% B to 77% B in 10 min, 77% B; Wave Length: 254; 220 nm; RT1 (min): 8.4, 9.43 (min); to afford (7S)-7-[(2S)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (85.5 mg, 21.31%) as a yellow solid.

LC-MS: M+H found: 468.1.

¹H NMR (400 MHz, DMSO-d6) δ 11.72 (s, 1H), 8.45 (d, J=5.5 Hz, 1H), 8.14 (s, 1H), 7.19 (s, 1H), 7.06 (d, J=5.5 Hz, J H), 6.76 (td, J=8.2, 5.9 Hz, 1H), 6.59 (dd, J=10.7, 8.5 Hz, 1H), 6.18 (d, J=8.3 Hz, 1H), 3.94 (s, 3H), 3.81-3.69 (m, 2H), 3.69-3.63 (m, 3H), 3.63-3.50 (m, 2H), 3.50-3.42 (m, 1H), 3.21 (t, J=10.6 Hz, 1H), 3.14-3.07 (m, 1H), 2.60 (s, 3H), 1.75 (dt, J=14.2, 4.7 Hz, 1H), 1.62 (dt, J=14.7, 8.0 Hz, 1H).

### Example 83. (7S)-3-[(3-chloro-2-methoxyphenyl) amino]-7-(methoxymethyl)-2-(pyridin-4-yl)-1H,5H,6H,7H-pyrrolo[3,2-c]pyridin-4-one (compound 747)

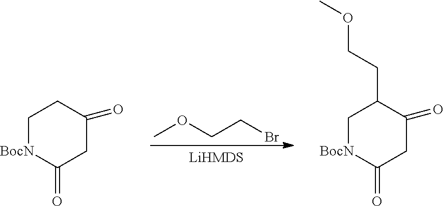

To a stirred solution of tert-butyl 2,4-dioxopiperidine-1-carboxylate (5 g, 23.449 mmol, 1.00 equiv) and 2-bromoethyl methyl ether (6.52 g, 46.898 mmol, 2.00 equiv) in THF (50 mL, 234.490 mmol) were added LiHMDS (9.81 g, 58.623 mmol, 2.50 equiv) dropwise at −70 degrees C. under N2 atmosphere. The mixture was stirred for 1.5 h at −70° C.˜−10° C. Then the mixture solution was adjusted to pH=5. The resulting mixture was extracted with EA (3×50 ml). The combined organic layers were washed with saturated NaCl solution (2×100 ml), dried over anhydrous Na₂SO₄. After filtration, the filtrate was concentrated under reduced pressure. The residue was purified by silica gel column chromatography, eluted with DCM/MeOH (99:1˜15:1) to afford tert-butyl 5-(2-methoxyethyl)-2,4-dioxopiperidine-1-carboxylate (4.7 g, 73.88%) as a yellow oil.

LC-MS: 2M+Na found: 565.15.

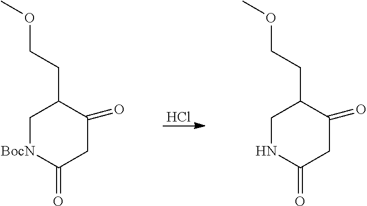 [SMILES: COCCC1CN(C(=O)OC(C)(C)C)C(=O)CC1=O ; Cl ; COCCC1CNC(=O)CC1=O]

To a stirred solution of tert-butyl 5-(2-methoxyethyl)-2,4-dioxopiperidine-1-carboxylate (1.5 g, 5.529 mmol, 1 equiv) in DCM (15 mL) was added HCl (gas) in 1,4-dioxane (7 mL, 230.389 mmol, 41.67 equiv) dropwise at 0° C. The resulting mixture was concentrated under reduced pressure to afford 5-(2-methoxyethyl) piperidine-2,4-dione (1.5 g, 158.48%), which was used directly in next step without further purification.

LC-MS: M+H found: 172.01.

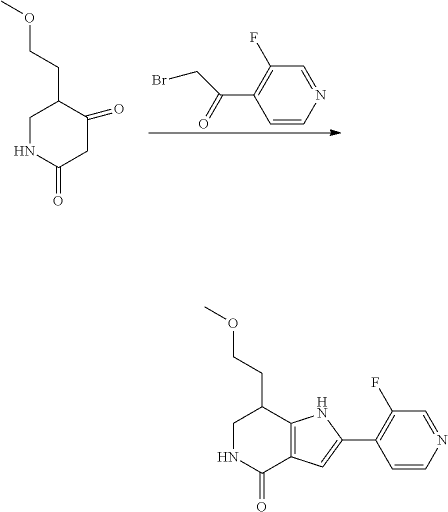 [SMILES: COCCC1CNC(=O)CC1=O ; O=C(CBr)c1ccncc1F ; COCCC1CNC(=O)c2cc(-c3ccncc3F)[nH]c21]

A solution of 5-(2-methoxyethyl) piperidine-2,4-dione (1.5 g, 8.762 mmol, 1.00 equiv), 2-bromo-1-(3-fluoropyridin-4-yl) ethanone (2.10 g, 9.632 mmol, 1.10 equiv) and NH₄OAc (4.73 g, 61.334 mmol, 7 equiv) in EtOH (20 mL) was stirred for 16 h at 50 degrees C. The mixture was allowed to cool down to RT. The resulting mixture was concentrated under reduced pressure. The residue was purified by silica gel column chromatography, eluted with DCM/MeOH (15:1) to afford 2-(3-fluoropyridin-4-yl)-7-(2-methoxyethyl)-1H,5H,6H,7H-pyrrolo[3,2-c] pyridin-4-one (1.6 g, 63.12%) as a yellow solid.

LC-MS: M+H found: 290.05.

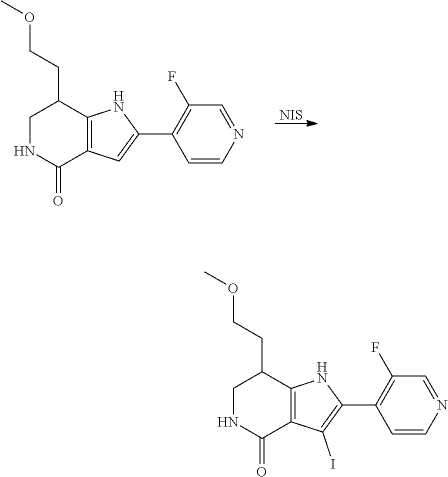

To a stirred solution of 2-(3-fluoropyridin-4-yl)-7-(2-methoxyethyl)-1H,5H,6H,7H-pyrrolo[3,2-c] pyridin-4-one (1000 mg, 3.456 mmol, 1.00 equiv) in DMF (10 mL) was added NIS (1166.49 mg, 5.184 mmol, 1.50 equiv) in portions at RT. The reaction was monitored by LCMS. When the reaction was complete, the resulting mixture was extracted with EA (3×20 ml). The combined organic layers were washed with saturated NaCl solution (2×40 ml), dried over anhydrous Na2SO4. After filtration, the filtrate was concentrated under reduced pressure. The residue was purified by silica gel column chromatography, eluted with DCM/MeOH (99:1˜15:1) to afford 2-(3-fluoropyridin-4-yl)-3-iodo-7-(2-methoxyethyl)-1H,5H,6H,7H-pyrrolo[3,2-c] pyridin-4-one (1.1 g, 76.65%) as a yellow solid.

LC-MS: M+H found: 416.10.

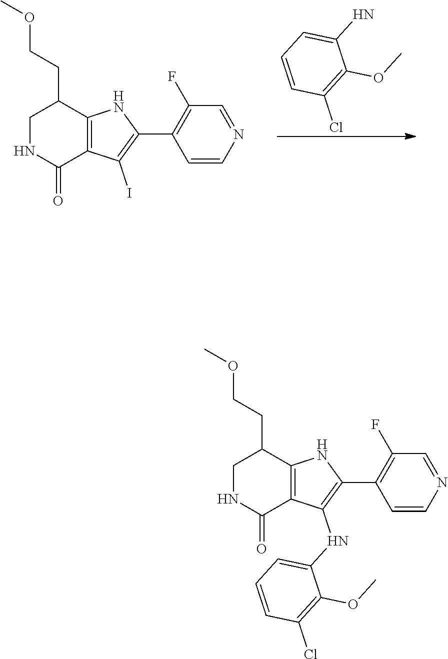

A mixture of 3-iodo-7-(2-methoxyethyl)-2-(pyridin-4-yl)-1H,5H,6H,7H-pyrrolo[3,2-c]pyridin-4-one (400 mg, 1.007 mmol, 1 equiv) and 3-chloro-2-methoxyaniline (317.41 mg, 2.014 mmol, 2 equiv) in DMF (4 mL) was treated with Cs2CO3 (984.31 mg, 3.021 mmol, 3 equiv) and EPhos Pd G4 (185.00 mg, 0.201 mmol, 0.2 equiv) for 3 h at 50° C. under nitrogen atmosphere. The mixture was allowed to cool down to RT. The resulting mixture was extracted with EA (3×10 ml). The combined organic layers were washed with saturated NaCl solution (2×20 ml), dried over anhydrous Na2SO4. After filtration, the filtrate was concentrated under reduced pressure. The residue was purified by silica gel column chromatography, eluted with DCM/MeOH (99:1˜15:1) to afford 3-[(3-chloro-2-methoxyphenyl) amino]-7-(2-methoxyethyl)-2-(pyridin-4-yl)-1H,5H,6H,7H-pyrrolo[3,2-c] pyridin-4-one (370 mg). The residue was purified by reverse flash chromatography with the following conditions: Column: YMC-Actus Triart C18, 30*150 mm, 5 μm; Mobile Phase A: Water (10 mmol/L NH4HCO3+0.1% NH3·H2O), Mobile Phase B: ACN; Flow rate: 60 mL/min; Gradient: 30% B to 60% B in 8 min, 60% B; Wave Length: 254/220 nm; RT1 (min): 7.48 to afford 3-[(3-chloro-2-methoxyphenyl) amino]-7-(2-methoxyethyl)-2-(pyridin-4-yl)-1H,5H,6H,7H-pyrrolo[3,2-c] pyridin-4-one (120 mg, 27.91%).

LC-MS: M+H found: 445.10.

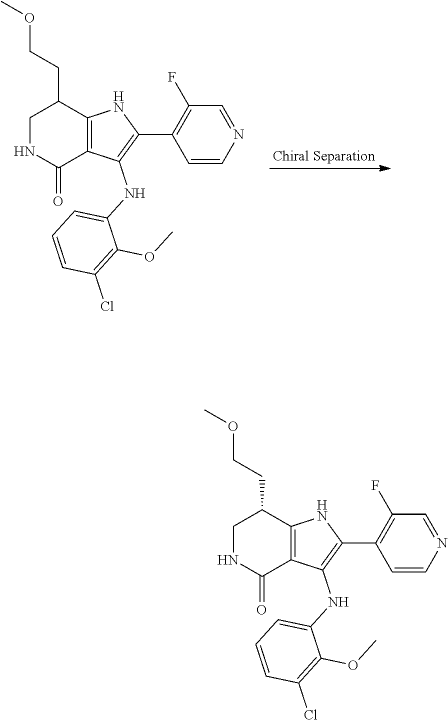 [SMILES: COCCC1CNC(=O)c2c1[nH]c(-c1ccncc1F)c2Nc1cccc(Cl)c1OC ; COCC[C@H]1CNC(=O)c2c1[nH]c(-c1ccncc1F)c2Nc1cccc(Cl)c1OC]

The crude product (170 mg, 0.383 mmol, 1.00 equiv) was purified by Prep-Chiral-HPLC (Column: CHIRALPAK IG-3, 4.6*50 mm 3 um; Mobile Phase A: (Hex:DCM=3:1)(0.1% DEA): IPA=70: 30; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c]pyridin-4-one (47.8 mg, 28.1%) as a light yellow solid.

LC-MS: M+H found 445.00.

¹H NMR (400 MHz, DMSO-d6) δ 11.49 (s, 1H), 8.51 (d, J=2.9 Hz, 1H), 8.29 (dd, J=5.1, 1.2 Hz, 1H), 7.59 (s, 1H), 7.45 (dd, J=6.8, 5.1 Hz, 1H), 7.19 (d, J=2.7 Hz, 1H), 6.73-6.57 (m, 2H), 6.13 (dd, J=7.3, 2.4 Hz, 1H), 3.86 (s, 3H), 3.59-3.40 (m, 3H), 3.30 (s, 3H), 3.24 (ddd, J=12.5, 6.0, 3.1 Hz, 1H), 3.09 (dt, J=8.4, 5.4 Hz, 1H), 2.09-1.95 (m, 1H), 1.78 (ddt, J=13.8, 8.1, 5.5 Hz, 1H).

### Example 84. (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (compound 746)

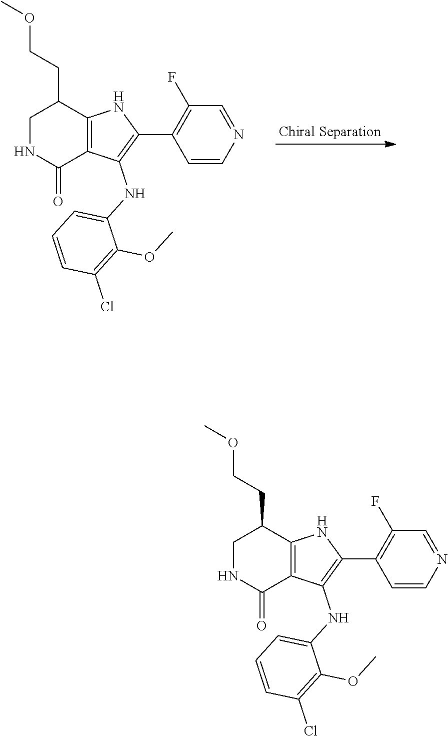 [SMILES: COCCC1CNC(=O)c2c1[nH]c(-c1ccncc1F)c2Nc1cccc(Cl)c1OC ; COCC[C@@H]1CNC(=O)c2c1[nH]c(-c1ccncc1F)c2Nc1cccc(Cl)c1OC]

The crude product (170 mg, 0.383 mmol, 1.00 equiv) was purified by Prep-Chiral-HPLC (Column: CHIRALPAK IG-3, 4.6*50 mm 3 um; Mobile Phase A: (Hex:DCM=3:1)(0.1% DEA):IPA=70:30; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (55.1 mg, 32.4%) as a light yellow solid.

LC-MS: M+H found 445.00.

¹H NMR (400 MHz, DMSO-d6) δ 11.49 (s, 1H), 8.51 (d, J=3.0 Hz, 1H), 8.29 (dd, J=5.1, 1.2 Hz, 1H), 7.59 (s, 1H), 7.44 (dd, J=6.8, 5.1 Hz, 1H), 7.18 (t, J=2.7 Hz, 1H), 6.73-6.60 (m, 2H), 6.13 (dd, J=7.3, 2.3 Hz, 1H), 3.86 (s, 3H), 3.57-3.41 (m, 3H), 3.30 (s, 3H), 3.24 (ddd, J=12.5, 6.1, 3.1 Hz, 1H), 3.09 (dq, J=11.0, 5.5 Hz, 1H), 2.07-1.95 (m, 1H), 1.78 (td, J=13.9, 5.6 Hz, 1H).

### Example 85. (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (compound 447a)

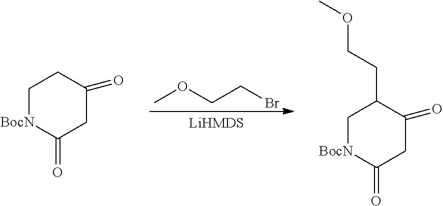

To a stirred solution of tert-butyl 2,4-dioxopiperidine-1-carboxylate (5 g, 23.449 mmol, 1.00 equiv) and 2-bromoethyl methyl ether (6.52 g, 46.898 mmol, 2.00 equiv) in THF (50 mL) were added LiHMDS (9.81 g, 58.623 mmol, 2.50 equiv) dropwise at −70 degrees C. under N2 atmosphere. The mixture was stirred for 1.5 h at −70° C.˜−10° C. Then the mixture solution was adjusted to pH=5. The resulting mixture was extracted with EA (3×50 ml). The combined organic layers were washed with saturated NaCl solution (2×100 ml), dried over anhydrous Na2SO4. After filtration, the filtrate was concentrated under reduced pressure. The residue was purified by silica gel column chromatography, eluted with DCM/MeOH (99:1˜15:1) to afford tert-butyl 5-(2-methoxyethyl)-2,4-dioxopiperidine-1-carboxylate (4.7 g, 73.88%) as a yellow oil.

LC-MS: 2M+Na found: 565.15.

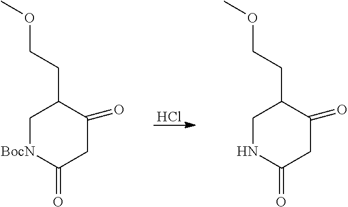 [SMILES: COCCC1CN(C(=O)OC(C)(C)C)C(=O)CC1=O ; Cl ; COCCC1CNC(=O)CC1=O]

To a stirred solution of tert-butyl 5-(2-methoxyethyl)-2,4-dioxopiperidine-1-carboxylate (1.5 g, 5.529 mmol, 1 equiv) in DCM (15 mL) was added HCl (gas) in 1,4-dioxane (7 mL, 230.389 mmol, 41.67 equiv) dropwise at 0° C. The resulting mixture was concentrated under reduced pressure to afford 5-(2-methoxyethyl) piperidine-2,4-dione (1.5 g, 158.48%), which was used directly in next step without further purification.

LC-MS: M+H found: 172.01.

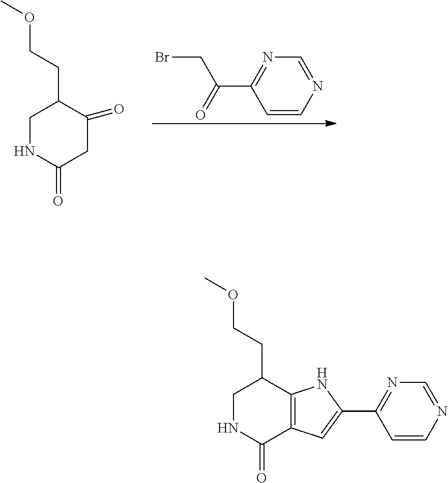 [SMILES: COCCC1CNC(=O)CC1=O ; O=C(CBr)c1ccncn1 ; COCCC1CNC(=O)c2cc(-c3ccncn3)[nH]c21]

A solution of 5-(2-methoxyethyl) piperidine-2,4-dione (1000 mg, 5.841 mmol, 1 equiv), 2-bromo-1-(pyrimidin-4-yl) ethanone (1291.65 mg, 6.425 mmol, 1.1 equiv) and NH₄OAc (3151.83 mg, 40.887 mmol, 7 equiv) in EtOH (10 mL) was stirred for 16 h at 50 degrees C. The mixture was allowed to cool down to RT. The resulting mixture was concentrated under reduced pressure. The residue was purified by silica gel column chromatography, eluted with DCM/MeOH (15:1) to afford 7-(2-methoxyethyl)-2-(pyrimidin-4-yl)-1H,5H,6H,7H-pyrrolo[3,2-c] pyridin-4-one (520 mg, 32.69%) as a yellow solid.

LC-MS: M+H found: 290.05.

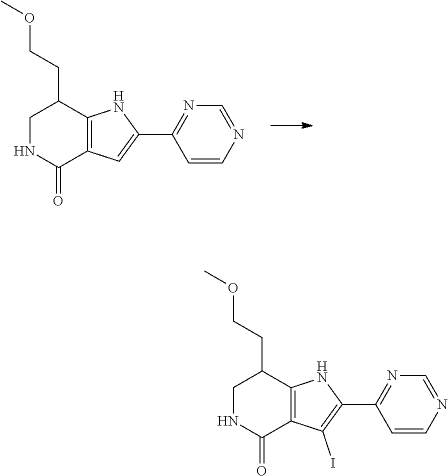 [SMILES: COCCC1CNC(=O)c2cc(-c3ccncn3)[nH]c21 ; COCCC1CNC(=O)c2c1[nH]c(-c1ccncn1)c2I]

To a stirred solution of 7-(2-methoxyethyl)-2-(pyrimidin-4-yl)-1H,5H,6H,7H-pyrrolo[3,2-c] pyridin-4-one (520 mg, 1.910 mmol, 1 equiv) in DMF (5 mL) was added NIS (644.45 mg, 2.865 mmol, 1.5 equiv) in portions at RT. The reaction was monitored by LCMS. When the reaction was complete, the resulting mixture was extracted with EA (3×20 ml). The combined organic layers were washed with saturated NaCl solution (2×40 ml), dried over anhydrous Na2SO4. After filtration, the filtrate was concentrated under reduced pressure. The residue was purified by silica gel column chromatography, eluted with DCM/MeOH (99:1˜15:1) to afford 3-iodo-7-(2-methoxyethyl)-2-(pyrimidin-4-yl)-1H,5H,6H,7H-pyrrolo[3,2-c] pyridin-4-one (530 mg, 69.70%) as a yellow solid.

LC-MS: M+H found: 398.85.

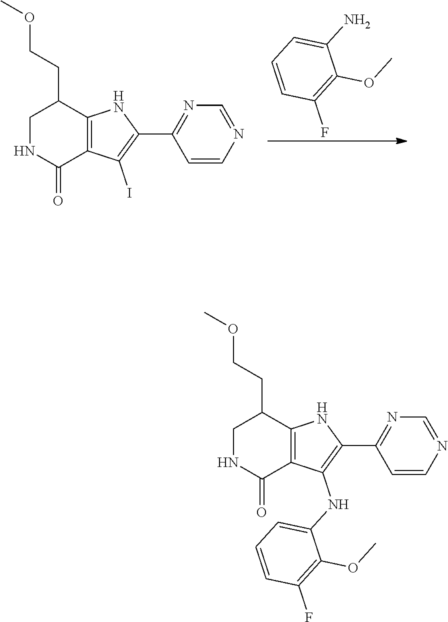

A solution of 3-iodo-7-(2-methoxyethyl)-2-(pyrimidin-4-yl)-1H,5H,6H,7H-pyrrolo[3,2-c] pyridin-4-one (200 mg, 0.502 mmol, 1 equiv) and 3-fluoro-2-methoxyaniline (141.78 mg, 1.004 mmol, 2 equiv) in DMF (2.5 mL) was treated with Cs2CO3 (490.93 mg, 1.506 mmol, 3 equiv) and EPhos Pd G4 (92.27 mg, 0.100 mmol, 0.2 equiv) for 3 h at 50° C. under nitrogen atmosphere. The mixture was allowed to cool down to RT. The resulting mixture was extracted with EA (3×10 ml). The combined organic layers were washed with saturated NaCl solution (2×20 ml), dried over anhydrous Na2SO4. After filtration, the filtrate was concentrated under reduced pressure. The residue was purified by silica gel column chromatography, eluted with DCM/MeOH (99:1˜15:1) to afford the crude product. The residue was purified by reverse flash chromatography with the following conditions: Column: XBridge Prep Amide OBD Column, 19*150 mm, 5 μm; Mobile Phase A: Water (10 mmol/L NH4HCO3), Mobile Phase B: ACN; Flow rate: 25 m/min; Gradient: 98% B to 50% B in 7 min, 50% B; RT1 (min): 3.82 to afford 3-[(3-fluoro-2-methoxyphenyl) amino]-7-(2-methoxyethyl)-2-(pyrimidin-4-yl)-1H,5H,6H,7H-pyrrolo[3,2-c] pyridin-4-one (35 mg, 16.94%) as a white solid.

LC-MS: M+H found: 412.30.

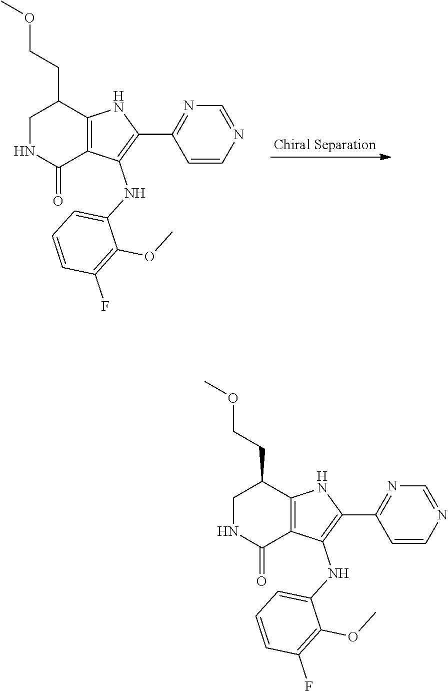 [SMILES: COCCC1CNC(=O)c2c1[nH]c(-c1ccncn1)c2Nc1cccc(F)c1OC ; COCC[C@@H]1CNC(=O)c2c1[nH]c(-c1ccncn1)c2Nc1cccc(F)c1OC]

The product 5 (35 mg, 0.079 mmol, 1.00 equiv) was purified by Prep-Chiral-HPLC (Column: CHIRALPAK IC-3, 4.6*50 mm 3 um; Mobile Phase A: Hex (0.1% DEA): EtOH=60: 40; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (8.9 mg, 25.4%) as a white solid.

LC-MS: M+H found: 412.30.

¹H NMR (300 MHz, DMSO-d6) δ 12.00 (s, 1H), 9.05 (d, J=1.4 Hz, 1H), 8.55 (d, J=5.6 Hz, 1H), 8.01 (s, 1H), 7.20 (d, J=5.9 Hz, 2H), 6.89-6.70 (m, 1H), 6.67-6.47 (m, 1H), 6.29-6.03 (m, 1H), 3.96 (s, 3H), 3.48 (d, J=21.4 Hz, 3H), 3.33-3.16 (m, 4H), 3.08 (s, 1H), 2.13-1.88 (m, 1H), 1.88-1.66 (m, 1H).

### Example 86. (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (compound 447b)

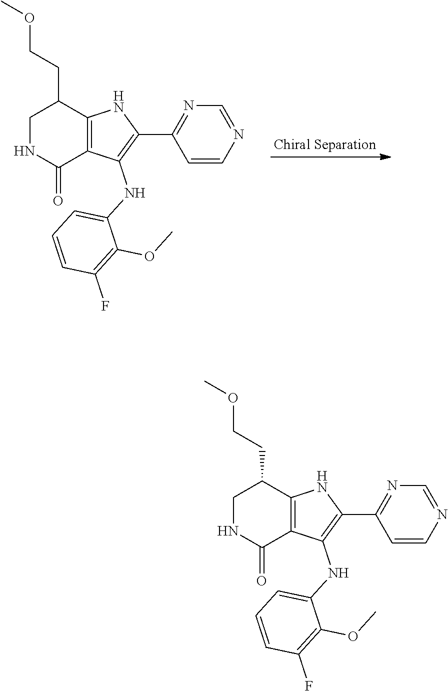 [SMILES: COCCC1CNC(=O)c2c1[nH]c(-c1ccncn1)c2Nc1cccc(F)c1OC ; COCC[C@H]1CNC(=O)c2c1[nH]c(-c1ccncn1)c2Nc1cccc(F)c1OC]

The product 5 (35 mg, 0.079 mmol, 1.00 equiv) was purified by Prep-Chiral-HPLC (Column: CHIRALPAK IC-3, 4.6*50 mm 3 um; Mobile Phase A: Hex (0.1% DEA):EtOH=60:40; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (8.4 mg, 24.0%) as a white solid.

LC-MS: M+H found 412.30.

¹H NMR (300 MHz, DMSO-d6) δ 12.00 (s, 1H), 9.05 (s, 1H), 8.56 (s, 1H), 8.01 (s, 1H), 7.20 (d, J=6.0 Hz, 2H), 6.88-6.70 (m, 1H), 6.62 (ddd, J=10.0, 8.4, 1.5 Hz, 1H), 6.18 (dt, J=8.2, 1.3 Hz, 1H), 3.96 (s, 3H), 3.48 (d, J=5.8 Hz, 3H), 3.25 (s, 4H), 3.09 (d, J=7.9 Hz, 1H), 2.02 (dt, J=12.8, 6.8 Hz, 1H), 1.85-1.69 (m, 1H).

### Example 87. (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (compound 489)

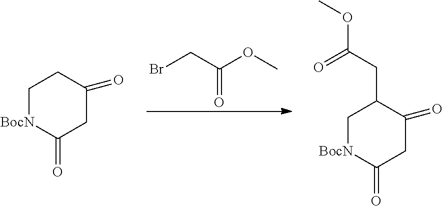

To a stirred solution of tert-butyl 2,4-dioxopiperidine-1-carboxylate (2500 mg, 11.724 mmol, 1.00 equiv) in THF (30 ml) was added LDA (3767.82 mg, 35.172 mmol, 3 equiv) dropwise at −70° C. under N2 atmosphere. After stirring for additional 30 min, methyl 2-bromoacetate (2690.28 mg, 17.586 mmol, 1.5 equiv) was added and continued to react for 2 h at −70° C. Then the mixture solution was adjusted to pH=5. The resulting mixture was extracted with EA (3×100 ml). The combined organic layers were washed with saturated NaCl solution (2×150 ml), dried over anhydrous Na2SO4. After filtration, the filtrate was concentrated under reduced pressure. The residue was purified by silica gel column chromatography, eluted with DCM/MeOH (99:1˜15:1) to afford tert-butyl 5-(2-methoxy-2-oxoethyl)-2,4-dioxopiperidine-1-carboxylate (820 mg, 24.52%) as a yellow oil.

LC-MS: 2M+Na found: 593.45.

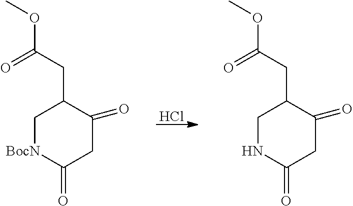 [SMILES: COC(=O)CC1CN(C(=O)OC(C)(C)C)C(=O)CC1=O ; Cl ; COC(=O)CC1CNC(=O)CC1=O]

To a stirred solution of tert-butyl 5-(2-methoxy-2-oxoethyl)-2,4-dioxopiperidine-1-carboxylate (820 mg, 2.874 mmol, 1 equiv) in DCM (8 mL) was added HCl (gas) in 1,4-dioxane (4 mL, 109.709 mmol, 38.17 equiv) dropwise at 0° C. The resulting mixture was concentrated under reduced pressure to afford methyl 2-(4,6-dioxopiperidin-3-yl) acetate (850 mg, 159.70%), which was used directly in next step without purification.

LC-MS: M+H found: 186.15.

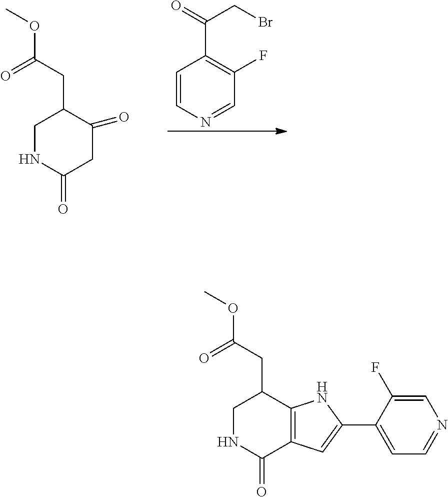 [SMILES: COC(=O)CC1CNC(=O)CC1=O ; O=C(CBr)c1ccncc1F ; COC(=O)CC1CNC(=O)c2cc(-c3ccncc3F)[nH]c21]

A solution of methyl 2-(4,6-dioxopiperidin-3-yl)acetate (850 mg, 4.590 mmol, 1 equiv), 2-bromo-1-(3-fluoropyridin-4-yl)ethanone (1200.92 mg, 5.508 mmol, 1.2 equiv) and NH4OAc (2476.76 mg, 32.130 mmol, 7 equiv) in EtOH (10 mL) was stirred for 16 h at 50 degrees C. The mixture was allowed to cool down to RT. The resulting mixture was concentrated under reduced pressure. The residue was purified by silica gel column chromatography, eluted with DCM/MeOH (15:1) to afford methyl 2-[2-(2-fluorophenyl)-4-oxo-1H,5H,6H,7H-pyrrolo[3,2-c]pyridin-7-yl]acetate (350 mg, 25.22%) as a yellow solid.

LC-MS: M+H found: 304.00.

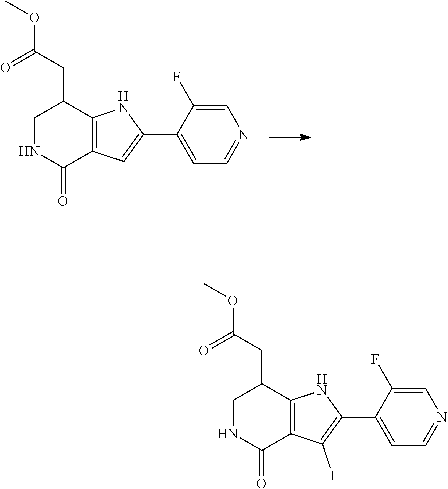 [SMILES: COC(=O)CC1CNC(=O)c2cc(-c3ccncc3F)[nH]c21 ; COC(=O)CC1CNC(=O)c2c1[nH]c(-c1ccncc1F)c2I]

To a stirred solution of methyl 2-[2-(3-fluoropyridin-4-yl)-4-oxo-1H,5H,6H,7H-pyrrolo[3,2-c] pyridin-7-yl] acetate (410 mg, 1.352 mmol, 1 equiv) in DMF (4 mL) was added NIS (456.21 mg, 2.028 mmol, 1.5 equiv) in portions at RT. The reaction was monitored by LCMS. When the reaction was complete, the resulting mixture was extracted with EA (3×20 ml). The combined organic layers were washed with saturated NaCl solution (2×40 ml), dried over anhydrous Na2SO4. After filtration, the filtrate was concentrated under reduced pressure. The residue was purified by silica gel column chromatography, eluted with DCM/MeOH (99:1˜15:1) to afford methyl 2-[2-(3-fluoropyridin-4-yl)-3-iodo-4-oxo-1H,5H,6H,7H-pyrrolo[3,2-c] pyridin-7-yl] acetate (420 mg, 72.39%) as a yellow solid.

LC-MS: M+H found: 430.15.

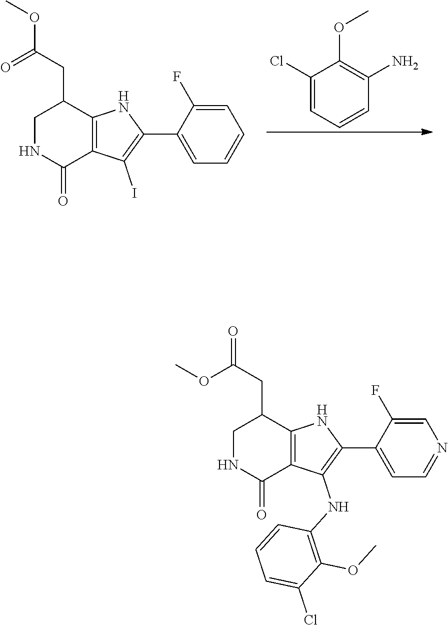

A solution of methyl 2-[2-(2-fluorophenyl)-3-iodo-4-oxo-1H,5H,6H,7H-pyrrolo[3,2-c]pyridin-7-yl] acetate (420 mg, 0.981 mmol, 1 equiv) and 3-chloro-2-methoxyaniline (309.16 mg, 1.962 mmol, 2 equiv) in DMF (4 mL) was treated with Cs₂CO₃ (958.73 mg, 2.943 mmol, 3 equiv) and EPhos Pd G4 (180.20 mg, 0.196 mmol, 0.2 equiv) for 3 h at 50° C. under nitrogen atmosphere. The mixture was allowed to cool down to RT. The resulting mixture was extracted with EA (3×10 ml). The combined organic layers were washed with saturated NaCl solution (2×20 ml), dried over anhydrous Na2SO4. After filtration, the filtrate was concentrated under reduced pressure. The residue was purified by silica gel column chromatography, eluted with DCM/MeOH (99.1-15:1) to afford methyl 2-{3-[(3-chloro-2-methoxyphenyl) amino]-2-(3-fluoropyridin-4-yl)-4-oxo-1H,5H,6H,7H-pyrrolo[3,2-c] pyridin-7-yl} acetate (400 mg, 88.87%) as a yellow solid.

LC-MS: M+H found: 459.00.

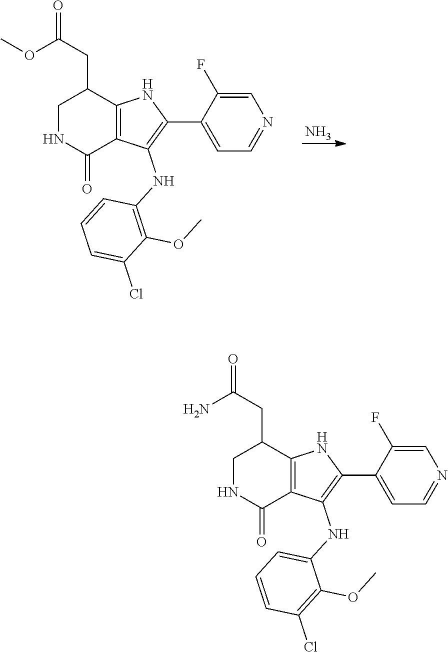

A solution of methyl 2-{3-[(3-chloro-2-methoxyphenyl) amino]-2-(3-fluoropyridin-4-yl)-4-oxo-1H,5H,6H,7H-pyrrolo[3,2-c] pyridin-7-yl} acetate (190 mg, 0.414 mmol, 1 equiv) in NH3 (g) in MeOH (0.2 mL, 7.046 mmol, 17.02 equiv) was stirred for 3 h at RT. The resulting mixture was concentrated under reduced pressure. The residue was purified by reverse flash chromatography with the following conditions: Column: YMC-Actus Triart C18, 30*150 mm, 5 μm; Mobile Phase A: Water (10 mmol/L NH4HCO3+0.1% NH3·H2O), Mobile Phase B: ACN; Flow rate: 60 mL/min; Gradient: 22% B to 42% B in 10 min, 42% B; Wave Length: 254/220 nm; RT1 (min): 9.56 to afford 2-[(7R)-3-[(3-chloro-2-methoxyphenyl)amino]-2-(3-fluoropyridin-4-yl)-4-oxo-1H,5H,6H,7H-pyrrolo[3,2-c]pyridin-7-yl]acetamide (50 mg, 27.21%) as a white solid.

LC-MS: M+H found 443.95.

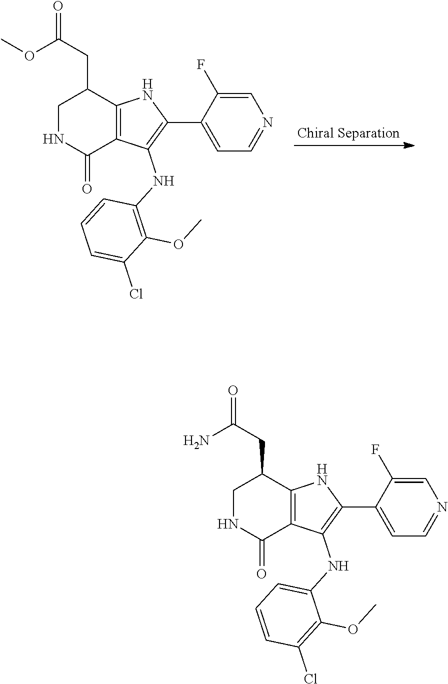 [SMILES: COC(=O)CC1CNC(=O)c2c1[nH]c(-c1ccncc1F)c2Nc1cccc(Cl)c1OC ; COc1c(Cl)cccc1Nc1c(-c2ccncc2F)[nH]c2c1C(=O)NC[C@H]2CC(N)=O]

The product 6 (50 mg, 0.113 mmol, 1.00 equiv) was purified by Prep-Chiral-HPLC (Column: CHIRALPAK IG-3, 4.6*50 mm, 3 um; Mobile Phase A: (Hex:DCM=3:1) (0.1% DEA): EtOH=80: 20; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (19.9 mg, 39.8%) as a white solid.

LC-MS: M+H found: 443.90.

¹H NMR (300 MHz, DMSO-d6) δ 11.62 (s, 1H), 8.51 (d, J=3.0 Hz, 1H), 8.29 (dd, J=5.1, 1.2 Hz, 1H), 7.70-7.38 (m, 3H), 7.19 (s, 1H), 7.08 (s, 1H), 6.74-6.58 (m, 2H), 6.14 (dd, J=6.8, 2.8 Hz, 1H), 3.87 (s, 3H), 3.55-3.36 (m, 2H), 3.24-3.13 (m, 1H), 2.46-2.38 (m, 2H).

### Example 88. (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (compound 488)

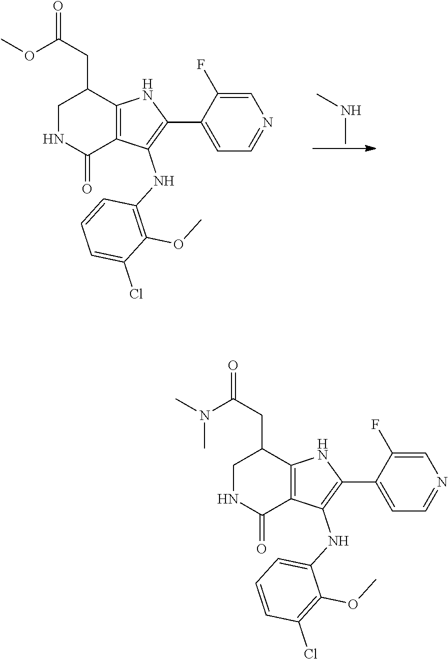

A solution of methyl 2-{3-[(3-chloro-2-methoxyphenyl) amino]-2-(3-fluoropyridin-4-yl)-4-oxo-1H,5H,6H,7H-pyrrolo[3,2-c] pyridin-7-yl} acetate (170 mg, 0.370 mmol, 1 equiv) and dimethylamine (2 M in aqueous) (0.1 mL, 1.508 mmol, 4.07 equiv) in MeOH (1 mL) was stirred for 3 h at 50° C. The resulting mixture was concentrated under reduced pressure. The residue was purified by reverse flash chromatography with the following conditions: Column: XBridge Prep OBD C18 Column, 30*150 mm, 5 μm; Mobile Phase A: Water (10 mmol/L NH4HCO3+0.1% NH3·H2O), Mobile Phase B: ACN; Flow rate: 60 mL/min; Gradient: 22% B to 52% B in 8 min, 52% B; Wave Length: 254; 220 nm; RT1 (min): 7.17 to afford 2-{3-[(3-chloro-2-methoxyphenyl) amino]-2-(3-fluoropyridin-4-yl)-4-oxo-1H,5H,6H,7H-pyrrolo[3,2-c] pyridin-7-yl}acetamide (50 mg, 30.41%)

LC-MS: M+H found 472.10.

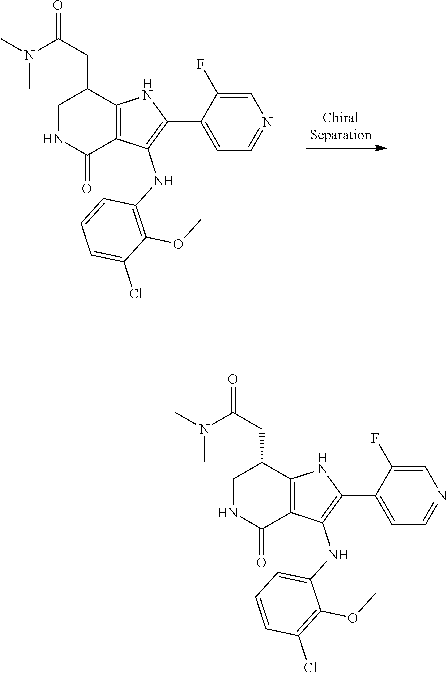 [SMILES: COc1c(Cl)cccc1Nc1c(-c2ccncc2F)[nH]c2c1C(=O)NCC2CC(=O)N(C)C ; COc1c(Cl)cccc1Nc1c(-c2ccncc2F)[nH]c2c1C(=O)NC[C@@H]2CC(=O)N(C)C]

The product 6 (50 mg, 0.106 mmol, 1.00 equiv) was purified by Prep-Chiral-HPLC (Column: CHIRALPAK IC-3, 4.6*50 mm 3 um; Mobile Phase A: Hex (0.1% DEA):EtOH=50:50; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (9.7 mg, 19.4%) as a white solid.

LC-MS: M+H found 472.10.

¹H NMR (400 MHz, DMSO-d6) δ 11.46 (s, 1H), 8.51 (d, J=3.0 Hz, 1H), 8.29 (d, J=5.1 Hz, 1H), 7.62 (s, 1H), 7.44 (t, J=5.9 Hz, 1H), 7.16 (s, 1H), 6.67 (d, J=7.9 Hz, 2H), 6.24-6.05 (m, 1H), 3.87 (s, 3H), 3.64-3.52 (m, 1H), 3.43 (s, 1H), 3.20 (d, J=13.1 Hz, 1H), 2.98 (s, 3H), 2.88 (s, 3H), 2.71 (d, J=6.9 Hz, 2H).

### Example 89. (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (compound 487)

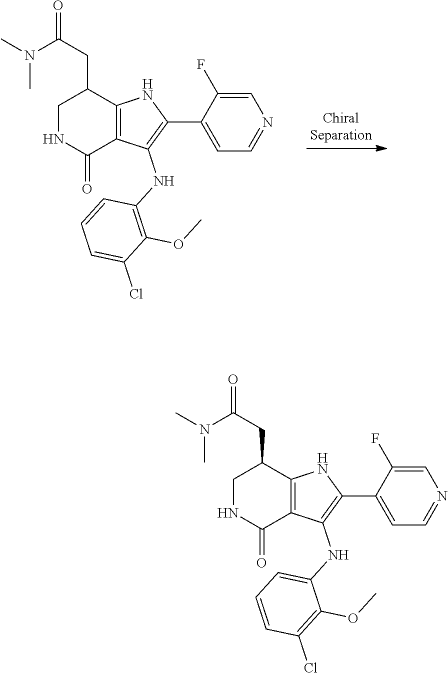 [SMILES: COc1c(Cl)cccc1Nc1c(-c2ccncc2F)[nH]c2c1C(=O)NCC2CC(=O)N(C)C ; COc1c(Cl)cccc1Nc1c(-c2ccncc2F)[nH]c2c1C(=O)NC[C@H]2CC(=O)N(C)C]

The product 6 (50 mg, 0.106 mmol, 1.00 equiv) was purified by Prep-Chiral-HPLC (Column: CHIRALPAK IC-3, 4.6*50 mm 3 um; Mobile Phase A: Hex (0.1% DEA):EtOH=50:50; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (9.7 mg, 19.4%) as a white solid.

LC-MS: M+H found 472.15.

¹H NMR (400 MHz, DMSO-d6) δ 11.46 (s, 1H), 8.52 (d, J=2.9 Hz, 1H), 8.29 (d, J=5.2 Hz, 1H), 7.62 (s, 1H), 7.44 (t, J=6.0 Hz, 1H), 7.16 (s, 1H), 6.67 (d, J=7.9 Hz, 2H), 6.15 (d, J=7.3 Hz, 1H), 3.87 (s, 3H), 3.57 (d, J=12.6 Hz, 1H), 3.43 (s, 1H), 3.22 (s, 1H), 2.98 (s, 3H), 2.88 (s, 3H), 2.71 (d, J=6.9 Hz, 2H).

### Example 90. (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (compound 474)

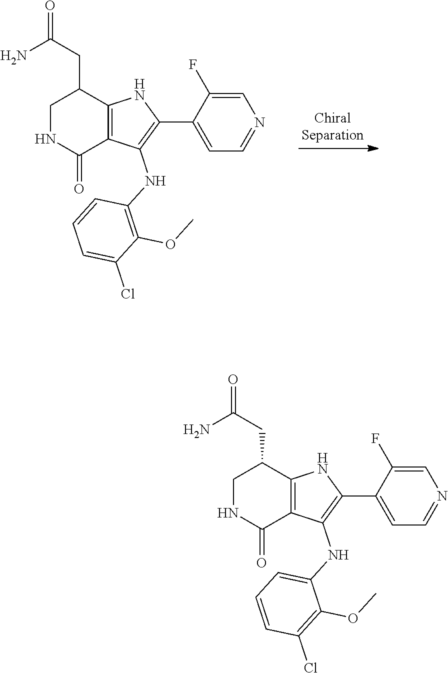 [SMILES: COc1c(Cl)cccc1Nc1c(-c2ccncc2F)[nH]c2c1C(=O)NCC2CC(N)=O ; COc1c(Cl)cccc1Nc1c(-c2ccncc2F)[nH]c2c1C(=O)NC[C@@H]2CC(N)=O]

The product 6 (50 mg, 0.113 mmol, 1.00 equiv) was purified by Prep-Chiral-HPLC (Column: CHIRALPAK IG-3, 4.6*50 mm, 3 um; Mobile Phase A: (Hex:DCM=3:1) (0.1% DEA): EtOH=80: 20; Flow rate: 1 mL/min; Gradient: 0% B to 0% B; Injection Volume: 5 ul mL) to afford (7S)-3-[(3-chloro-2-methoxyphenyl) amino]-7-(methoxymethyl)-2-(pyridin-4-yl)-1H,5H,6H,7H-pyrrolo[3,2-c] pyridin-4-one (19.7 mg, 39.4%) as a white solid.

LC-MS: M+H found: 443.90.

¹H NMR (300 MHz, DMSO-d6) δ 11.62 (s, 1H), 8.51 (d, J=3.0 Hz, 1H), 8.29 (dd, J=5.2, 1.2 Hz, 1H), 7.60 (s, 1H), 7.56-7.49 (m, 1H), 7.44 (dd, J=6.9, 5.1 Hz, 1H), 7.19 (s, 1H), 7.08 (s, 1H), 6.75-6.59 (m, 2H), 6.14 (dd, J=6.9, 2.8 Hz, 1H), 3.87 (s, 3H), 3.54-3.37 (m, 2H), 3.18 (dt, J=11.9, 4.5 Hz, 1H), 2.45-2.31 (m, 2H).

### Example 91. 3′-[(3-chloro-2-methoxyphenyl)amino]-2′-(3-fluoropyridin-4-yl)-1-(prop-2-enoyl)-5′,6′-dihydro-1′H-spiro[piperidine-4,4′-pyrrolo[2,3-c]pyridin]-7′-one (compound 831)

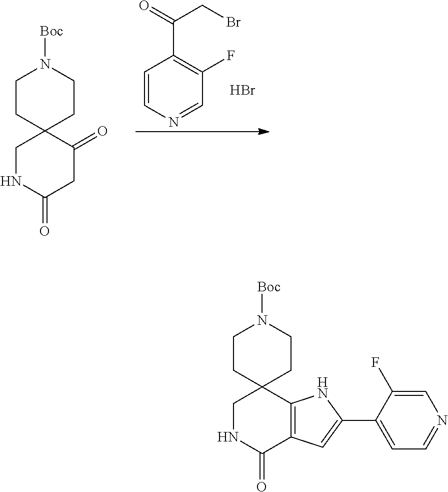 [SMILES: CC(C)(C)OC(=O)N1CCC2(CC1)CNC(=O)CC2=O ; O=C(CBr)c1ccncc1F ; Br ; CC(C)(C)OC(=O)N1CCC2(CC1)CNC(=O)c1cc(-c3ccncc3F)[nH]c12]

A solution of tert-butyl 3,5-dioxo-2,9-diazaspiro[5.5]undecane-9-carboxylate (20 mg, 0.071 mmol, 1 equiv) and ammonium acetate (18.82 mg, 0.319 mmol, 4.5 equiv), 2-bromo-1-(3-fluoropyridin-4-yl)ethanone (23.17 mg, 0.106 mmol, 1.5 equiv) in EtOH (3 mL) was stirred at 50° C. under N2 atmosphere for 6 h. The resulting mixture was extracted with EA (3×35 ml). The combined organic layers were washed with brine (3×20 ml), dried over anhydrous Na2SO4. After filtration, the filtrate was concentrated under reduced pressure. The residue was purified by Prep-TLC (DCM:MeOH=15:1) to afford tert-butyl 2′-(3-fluoropyridin-4-yl)-4′-oxo-5′,6′-dihydro-1′H-spiro[piperidine-4,7′-pyrrolo[3,2-c]pyridine]-1-carboxylate (15 mg, 53.86%) as a yellow solid.

LC-MS: (M+H)⁺ found: 401.95.

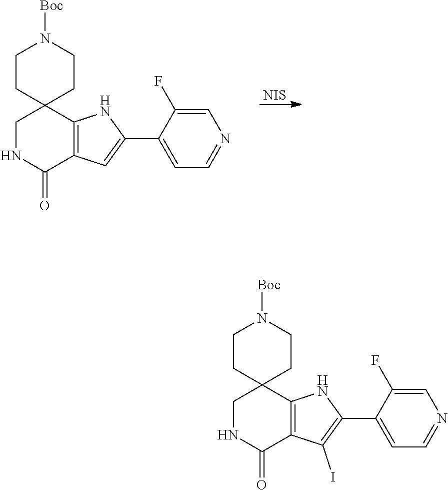

To a solution of tert-butyl 2′-(3-fluoropyridin-4-yl)-4′-oxo-5′,6′-dihydro-1′H-spiro[piperidine-4,7′-pyrrolo[3,2-c]pyridine]-1-carboxylate (500 mg, 1.249 mmol, 1.0 equiv) in DMF (2.3 mL) was added NIS (337.09 mg, 1.499 mmol, 1.2 equiv) in portions at RT, the resulting mixture was stirred for 2 h at RT under nitrogen atmosphere. The resulting mixture was diluted with water (20 mL), extracted with EA (3×30 ml). The combined organic layers were washed with brine (1×30 ml), dried over anhydrous Na2SO4. After filtration, the filtrate was concentrated under reduced pressure. The residue was purified by Prep-TLC (DCM:MeOH=20:1) to afford tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (406 mg, 61.86%) as a yellow solid.

LC-MS: (M−H)⁻ found: 526.95.

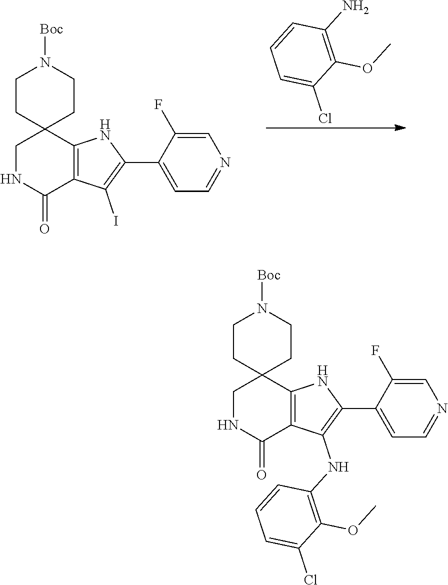

To a mixture of tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (30 mg, 0.057 mmol, 1 equiv) and 3-chloro-2-methoxyaniline (17.97 mg, 0.114 mmol, 2 equiv) and Cs2CO3 (46.43 mg, 0.143 mmol, 2.5 equiv) in DMF (1 mL) were added EPhos Pd G4 (10.47 mg, 0.011 mmol, 0.2 equiv) and EPhos (6.10 mg, 0.011 mmol, 0.2 equiv), the resulting mixture was stirred at 50° C. under N2 atmosphere for 3 h. The resulting mixture was diluted with water (20 mL), extracted with EA (3×30 mL). The combined organic layers were washed with brine (1×120 ml), dried over anhydrous Na2SO4. After filtration, the filtrate was concentrated under reduced pressure. The residue was purified by Prep-TLC (DCM:MeOH=20:1) to afford tert-butyl 3′-[(3-chloro-2-methoxyphenyl)amino]-2′-(3-fluoropyridin-4-yl)-4′-oxo-5′,6′-dihydro-1′H-spiro[piperidine-4,7′-pyrrolo[3,2-c]pyridine]-1-carboxylate (17 mg, 54.55%) as a brown solid.

LC-MS: (M+H)⁺ found: 556.20.

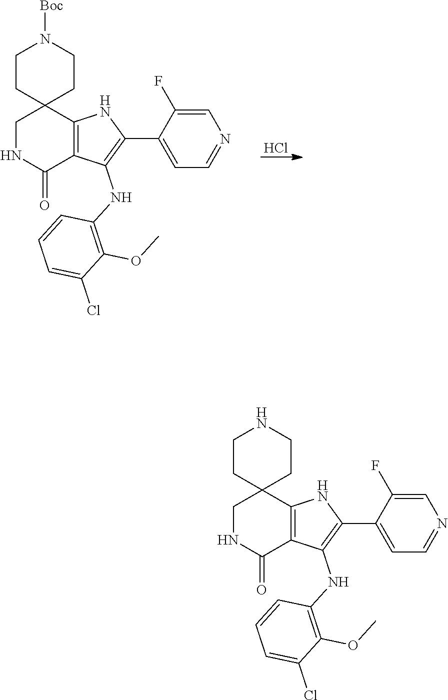 [SMILES: COc1c(Cl)cccc1Nc1c(-c2ccncc2F)[nH]c2c1C(=O)NCC21CCN(C(=O)OC(C)(C)C)CC1 ; Cl ; COc1c(Cl)cccc1Nc1c(-c2ccncc2F)[nH]c2c1C(=O)NCC21CCNCC1]

A solution of tert-butyl 3′-[(3-chloro-2-methoxyphenyl)amino]-2′-(3-fluoropyridin-4-yl)-4′-oxo-5′,6′-dihydro-1′H-spiro[piperidine-4,7′-pyrrolo[3,2-c]pyridine]-1-carboxylate (300 mg, 0.540 mmol, 1 equiv) in hydrogen chloride in 1,4-dioxane (50.00 mL, 4M) was stirred at 25° C. for 1 h. The resulting mixture was concentrated under vacuum. The residue was basified to pH=8 with NaHCO₃. The resulting mixture was extracted with EA (3×20 ml). The combined organic layers were washed with brine (50 ml), dried over anhydrous Na₂SO₄. After filtration, the filtrate was concentrated under reduced pressure to afford 3′-[(3-chloro-2-methoxyphenyl)amino]-2′-(3-fluoropyridin-4-yl)-5′,6′-dihydro-1′H-spiro[piperidine-4,7′-pyrrolo[3,2-c]pyridin]-4′-one) (206 mg, 84.86%) as a yellow solid.

LC-MS: (M+H)⁺ found: 412.90.

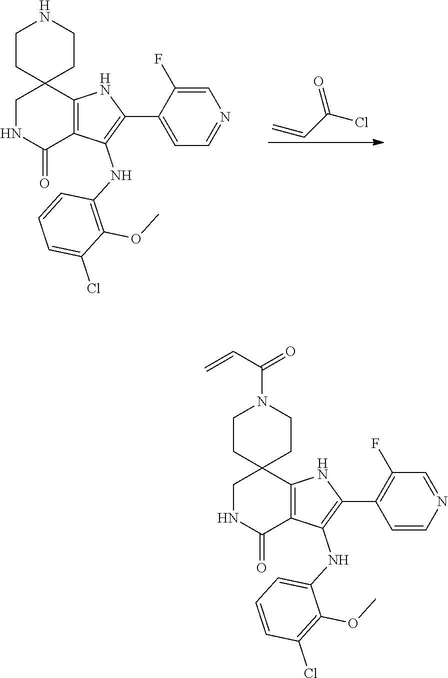

To a stirred solution of 3′-[(3-chloro-2-methoxyphenyl)amino]-2′-(3-fluoropyridin-4-yl)-5′,6′-dihydro-1′H-spiro[piperidine-4,4′-pyrrolo[2,3-c]pyridin]-7′-one (70 mg, 0.154 mmol, 1.00 equiv) in THF (3.5 mL)/aqueous NaHCO3solution (3.5 mL) was added prop-2-enoyl bromide (12.43 mg, 0.092 mmol, 0.60 equiv) dropwise at 0° C., the resulting mixture was stirred for 2 h. The resulting mixture was extracted with EA (3×20 ml). The combined organic layers were washed with brine (50 ml), dried over anhydrous Na2SO4. After filtration, the filtrate was concentrated under reduced pressure, the crude product was purified by Prep-HPLC with the following conditions (Column: YMC-Actus Triart C18, 30*150 mm, 5 μm; Mobile Phase A: Water (10 mmol/L NH4HCO3+0.1% NH3·H2O), Mobile Phase B: ACN; Flow rate: 60 mL/min; Gradient: 20% B to 60% B in 10 min, 60% B; Wave Length: 254/220 nm; RT1 (min): 8.47) to afford 3′-[(3-chloro-2-methoxyphenyl)amino]-2′-(3-fluoropyridin-4-yl)-1-(prop-2-enoyl)-5′,6′-dihydro-1′H-spiro[piperidine-4,4′-pyrrolo[2,3-c]pyridin]-7′-one (5.5 mg, 6.93%) as a yellow solid. LC-MS: (M+H)⁺ found: 509.95.

¹H NMR (300 MHz, DMSO-d6) δ 11.44 (s, 1H), 8.42 (d, J=49.1 Hz, 1H), 7.70 (s, 1H), 7.41 (d, J=28.5 Hz, 1H), 6.88 (dd, J=16.6, 10.4 Hz, 1H), 6.73-6.54 (m, 2H), 6.20-6.03 (m, 2H), 5.71 (dd, J=10.4, 2.5 Hz, 1H), 4.43 (d, J=12.2 Hz, 1H), 4.06 (d, J=13.5 Hz, 1H), 3.84 (s, 3H), 3.53 (s, 2H), 2.85 (s, 1H), 1.95 (s, 2H), 1.77 (s, 2H).

### Example 92. 3′-[(3-chloro-2-methoxyphenyl)amino]-2′-(3-fluoropyridin-4-yl)-1-(prop-2-enoyl)-5′,6′-dihydro-1′H-spiro[piperidine-4,4′-pyrrolo[2,3-c]pyridin]-7′-one (compound 830)

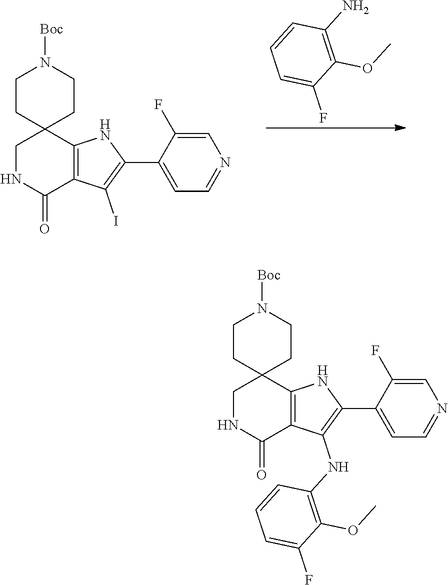

A mixture of tert-butyl 2′-(3-fluoropyridin-4-yl)-3′-iodo-4′-oxo-5′,6′-dihydro-1′H-spiro[piperidine-4,7′-pyrrolo[3,2-c]pyridine]-1-carboxylate (50 mg, 0.097 mmol, 1 equiv) and 3-chloro-2-methoxyaniline (17.97 mg, 0.194 mmol, 2 equiv) and Cs₂CO₃ (88.43 mg, 0.243 mmol, 2.5 equiv), EPhos Pd G4 (10.47 mg, 0.011 mmol, 0.2 equiv) and EPhos (6.10 mg, 0.011 mmol, 0.2 equiv) in DMF (1 mL) were stirred at 50° C. under N2 atmosphere for 2 h. The resulting mixture was extracted with EA (3×30). The combined organic layers were washed with brine (3×20 ml), dried over anhydrous Na₂SO₄. After filtration, the filtrate was concentrated under reduced pressure. The residue was purified by Prep-TLC (DCM:MeOH=20:1) to afford tert-butyl 3′-[(3-chloro-2-methoxyphenyl)amino]-2′-(3-fluoropyridin-4-yl)-4′-oxo-5′,6′-dihydro-1′H-spiro[piperidine-4,7′-pyrrolo[3,2-c]pyridine]-1-carboxylate (25 mg, 49.55%) as a brown solid.

LC-MS: (M+H)⁺ found: 541.32.

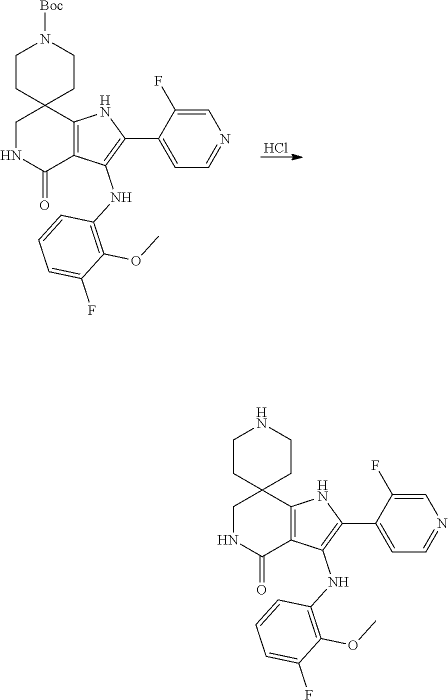 [SMILES: COc1c(F)cccc1Nc1c(-c2ccncc2F)[nH]c2c1C(=O)NCC21CCN(C(=O)OC(C)(C)C)CC1 ; Cl ; COc1c(F)cccc1Nc1c(-c2ccncc2F)[nH]c2c1C(=O)NCC21CCNCC1]

A solution of tert-butyl 3′-[(3-chloro-2-methoxyphenyl) amino]2′-(3-fluoropyridin-4-yl)-4′-oxo-5′,6′-dihydro-1′H-spiro[piperidine-4,7′-pyrrolo[3,2-c]pyridine]-1-carboxylate (300 mg, 0.540 mmol, 1 equiv) in hydrogen chloride dioxane (5.00 mL) was stirred at 25° C. for 2 h. The resulting mixture was concentrated under vacuum. The residue was basified to pH 8 with NaHCO₃. The resulting mixture was extracted with EA (3×20 ml). The combined organic layers were washed with brine (50 ml), dried over anhydrous Na₂SO₄. After filtration, the filtrate was concentrated under reduced pressure, to afford 3′-[(3-chloro-2-methoxyphenyl)amino]-2′-(3-fluoropyridin-4-yl)-5′,6′-dihydro-1′H-spiro[piperidine-4,7′-pyrrolo[3,2-c]pyridin]-4′-one) (210.0 mg, 87.55%) as a yellow solid.

LC-MS: (M+H)⁺ found: 440.10.

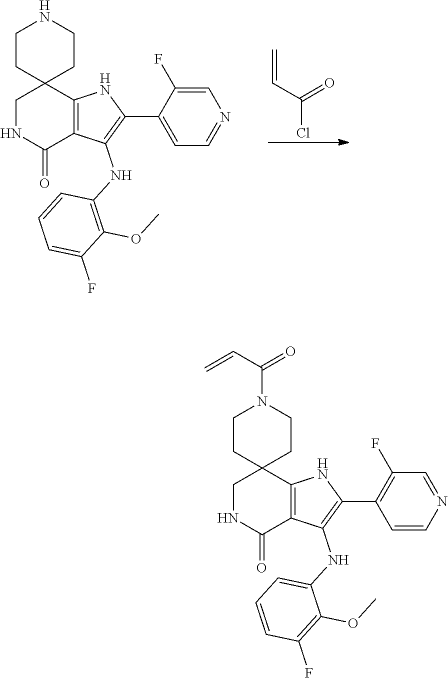

To a stirred solution of 3′-[(3-chloro-2-methoxyphenyl)amino]-2′-(3-fluoropyridin-4-yl)-5′,6′-dihydro-1′H-spiro[piperidine-4,4′-pyrrolo[2,3-c]pyridin]-7′-one (90 mg, 0.174 mmol, 1.00 equiv) in THF (3.5 mL)/NaHCO₃ (aq, 3.5 mL) was added prop-2-enoyl bromide (12.43 mg, 0.092 mmol, 0.60 equiv) dropwise at 0° C. under N2 atmosphere. The resulting mixture was extracted with EA (3×20 ml). The combined organic layers were washed with brine (50 ml), dried over anhydrous Na2SO4. After filtration, the filtrate was concentrated under reduced pressure, the crude product was purified by Prep-HPLC with the following conditions (Column: YMC-Actus Triart C18, 30*150 mm, 5 μm; Mobile Phase A: Water (10 mmol/L NH4HCO3+0.1% NH3·H2O), Mobile Phase B: ACN; Flow rate: 60 mL/min; Gradient: 20% B to 60% B in 10 min, 60% B; Wave Length: 254/220 nm; RT1 (min): 8.47) to afford 3′-[(3-chloro-2-methoxyphenyl)amino]-2′-(3-fluoropyridin-4-yl)-1-(prop-2-enoyl)-5′,6′-dihydro-1′H-spiro[piperidine-4,4′-pyrrolo[2,3-c]pyridin]-7′-one (19.1 mg, 21.23%) as a yellow solid.

LC-MS: (M+H)⁺ found: 509.95.

¹H NMR (300 MHz, DMSO-d6) δ 11.44 (s, 1H), 8.42 (d, J=49.1 Hz, 1H), 7.70 (s, 1H), 7.41 (d, J=28.5 Hz, 1H), 6.88 (dd, J=16.6, 10.4 Hz, 1H), 6.73-6.54 (m, 2H), 6.20-6.03 (m, 2H), 5.71 (dd, J=10.4, 2.5 Hz, 1H), 4.43 (d, J=12.2 Hz, 1H), 4.06 (d, J=13.5 Hz, 1H), 3.84 (s, 3H), 3.53 (s, 2H), 2.85 (s, 1H), 1.95 (s, 2H), 1.77 (s, 2H).

### Example 93. 2-(2-aminopyrimidin-4-yl)-3-[(3-chloro-2-methoxyphenyl)amino]-6-(hydroxymethyl)-1H,5H,6H,7H-pyrrolo[3,2-c]pyridin-4-one (compound 751)

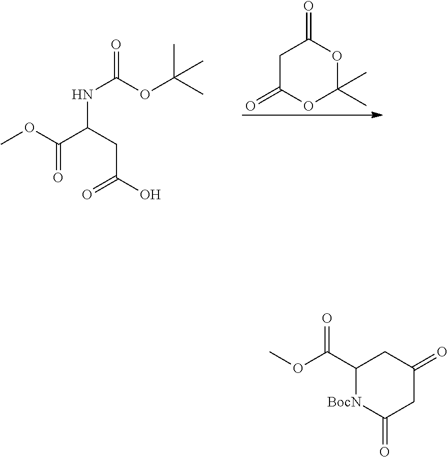

A solution of 3-[(tert-butoxycarbonyl)amino]-4-methoxy-4-oxobutanoic acid (8 g, 32.356 mmol, 1 equiv), DMAP (5929.50 mg, 48.534 mmol, 1.5 equiv), EDCI (9304.06 mg, 48.534 mmol, 1.5 equiv) and meldrum's acid (4663.39 mg, 32.356 mmol, 1.0 equiv) in DCM (80 mL) was stirred for RT at 3 h and washed with KHSO4 solution (1M, 200 mL×2), The organic layer was dried over sodium sulfate, filtered, and concentrated under reduced pressure. The residue was dissolved in EA (200 mL) and heated to reflux for 5 hours. After cooling down to room temperature, the mixture was washed with 1N KHSO4 solution (100 mL) and brine (10 mL), dried over sodium sulfate, filtered and evaporated to give a crude product, which was slurred in a mixture of EA and PE (100 mL, 10% V/V) to yield 1-tert-butyl 2-methyl 4,6-dioxopiperidine-1,2-dicarboxylate (4 g, 45.57%).

LC-MS: M+H found: 216.

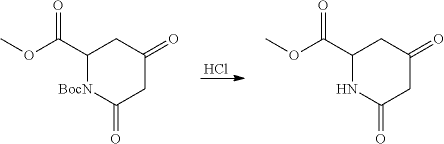 [SMILES: COC(=O)C1CC(=O)CC(=O)N1C(=O)OC(C)(C)C ; Cl ; COC(=O)C1CC(=O)CC(=O)N1]

A solution of 1-tert-butyl 2-methyl 4,6-dioxopiperidine-1,2-dicarboxylate (4 g, 14.746 mmol, 1 equiv) in DCM (10 mL) was treated with HCl (gas) in 1,4-dioxane (10 mL) for 2 h at rt. The resulting mixture was concentrated under reduced pressure. The crude product was used directly in next step.

LC-MS: M+H found: 171.

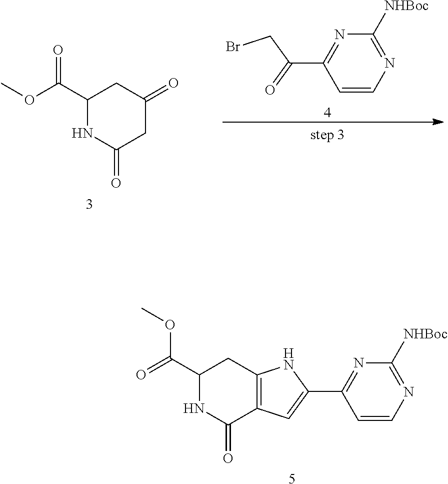

A solution of methyl 4,6-dioxopiperidine-2-carboxylate (2.5 g, 14.607 mmol, 1 equiv), tert-butyl N-[4-(2-bromoacetyl)pyrimidin-2-yl]carbamate (5541.65 mg, 17.528 mmol, 1.2 equiv) and NH₄OAc (5629.72 mg, 73.035 mmol, 5.00 equiv) in EtOH (25 mL) was stirred for 16 h at 50° C. The resulting mixture was concentrated under reduced pressure. The residue was purified by silica gel column chromatography, eluted with DCM/MeOH (20:1) to afford methyl 2-{2-[(tert-butoxycarbonyl)amino]pyrimidin-4-yl}-4-oxo-1H,5H,6H,7H-pyrrolo[3,2-c]pyridine-6-carboxylate (2.0 g, 35.34%) as a yellow solid.

LC-MS: M+H found: 488.

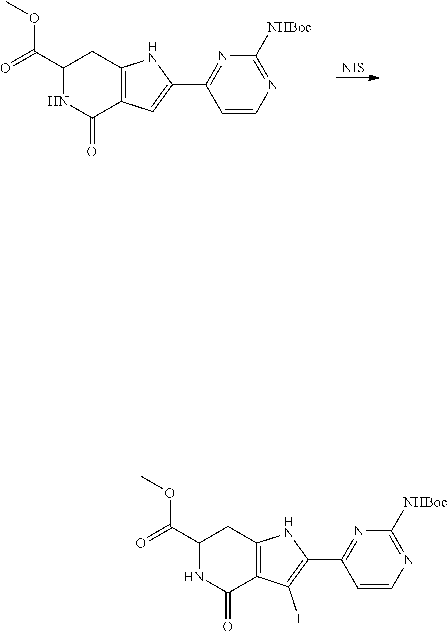 [SMILES: COC(=O)C1Cc2[nH]c(-c3ccnc(NC(=O)OC(C)(C)C)n3)cc2C(=O)N1 ; "NIS" ; COC(=O)C1Cc2[nH]c(-c3ccnc(NC(=O)OC(C)(C)C)n3)c(I)c2C(=O)N1]

To a mixture of methyl 2-{2-[(tert-butoxycarbonyl)amino]pyrimidin-4-yl}-4-oxo-1H,5H,6H,7H-pyrrolo[3,2-c]pyridine-6-carboxylate (2 g, 5.163 mmol, 1 equiv) in DMF (20 mL) was added NIS (1742.29 mg, 7.745 mmol, 1.5 equiv), the reaction mixture was stirred at 50° C. for 2 h. The reaction was quenched with saturated Na2S2O3, then extracted with DCM. The organic phase was concentrated under vacuum. The residue was purified by flash chromatography (9% MeOH in DCM) to give methyl 2-{2-[(tert-butoxycarbonyl)amino]pyrimidin-4-yl}-3-iodo-4-oxo-1H,5H,6H,7H-pyrrolo[3,2-c]pyridine-6-carboxylate (1.7 g, 64.15%) as a light yellow solid.

LC-MS: M+H found: 514.

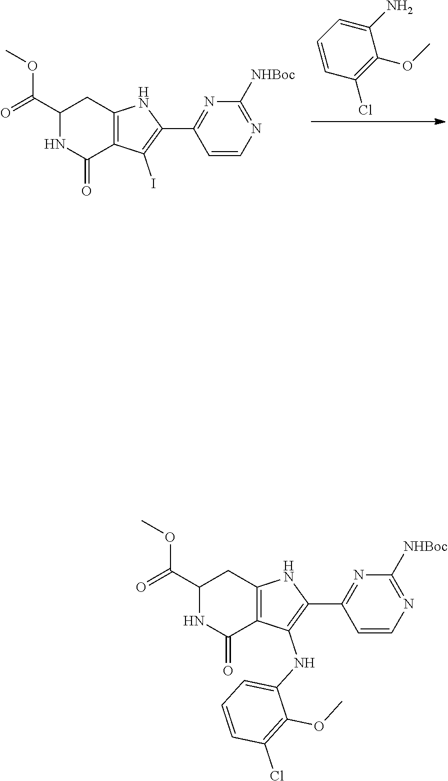

Into a 20-mL sealed tube was placed methyl 2-{2-[(tert-butoxycarbonyl)amino]pyrimidin-4-yl}-3-iodo-4-oxo-1H,5H,6H,7H-pyrrolo[3,2-c]pyridine-6-carboxylate (1.7 g, 3.312 mmol, 1.0 equiv) in dioxane (17 mL), 3-chloro-2-methoxyaniline (0.57 g, 3.643 mmol, 1.1 equiv), EPhos (0.89 g, 1.656 mmol, 0.5 equiv), EPhos Pd G4 (1.52 g, 1.656 mmol, 0.5 equiv) and Cs₂CO₃ (2.16 g, 6.624 mmol, 2 equiv). The resulting solution was stirred at 50° C. for 2 h. The reaction mixture was added to the ice water and extracted with EA. The organic phase was concentrated under vacuum, the residue was purified by Prep-Flash-HPLC with following conditions (9% MeOH in DCM). This resulted in methyl 2-{2-[(tert-butoxycarbonyl)amino]pyrimidin-4-yl}-3-[(3-chloro-2-methoxyphenyl)amino]-4-oxo-1H,5H,6H,7H-pyrrolo[3,2-c]pyridine-6-carboxylate (1.4 g, 77.85%) as a light yellow solid.

LC-MS: M+H found: 543.

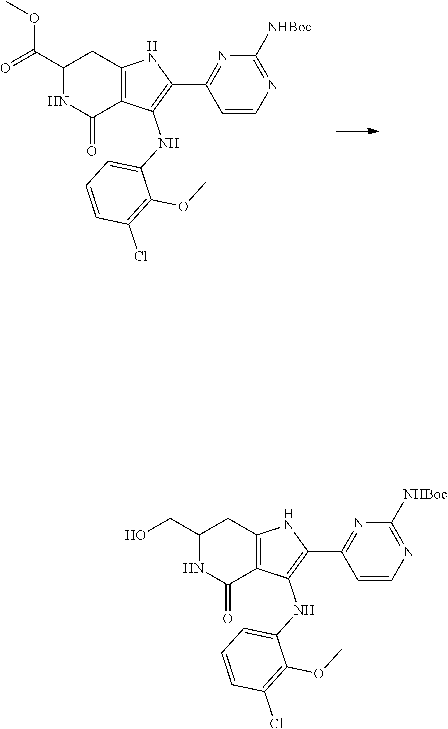 [SMILES: COC(=O)C1Cc2[nH]c(-c3ccnc(NC(=O)OC(C)(C)C)n3)c(Nc3cccc(Cl)c3OC)c2C(=O)N1 ; COc1c(Cl)cccc1Nc1c(-c2ccnc(NC(=O)OC(C)(C)C)n2)[nH]c2c1C(=O)NC(CO)C2]

A solution of methyl 2-{2-[(tert-butoxycarbonyl)amino]pyrimidin-4-yl}-3-[(3-chloro-2-methoxyphenyl)amino]-4-oxo-1H,5H,6H,7H-pyrrolo[3,2-c]pyridine-6-carboxylate (900 mg, 1.658 mmol, 1 equiv) in THF (10 mL) at −50° C. under nitrogen atmosphere followed by the addition of Diisobutylaluminium hydride (1.2M, 9 eq, 10.44 mL) dropwise at −50° C. The resulting mixture was stirred for 1 h at RT under N2 atmosphere. The reaction was quenched with saturated NH4Cl and 10% NaOH(aq). The aqueous layer was extracted with EA (3×10 mL). The residue was purified by silica gel column chromatography, eluted with DCM/MEOH (5:1) to afford tert-butyl N-(4-{3-[(3-chloro-2-methoxyphenyl)amino]-6-(hydroxymethyl)-4-oxo-1H,5H,6H,7H-pyrrolo[3,2-c]pyridin-2-yl}pyrimidin-2-yl)carbamate (300 mg, 35.15%) as a light yellow solid.

LC-MS: M+H found: 515.

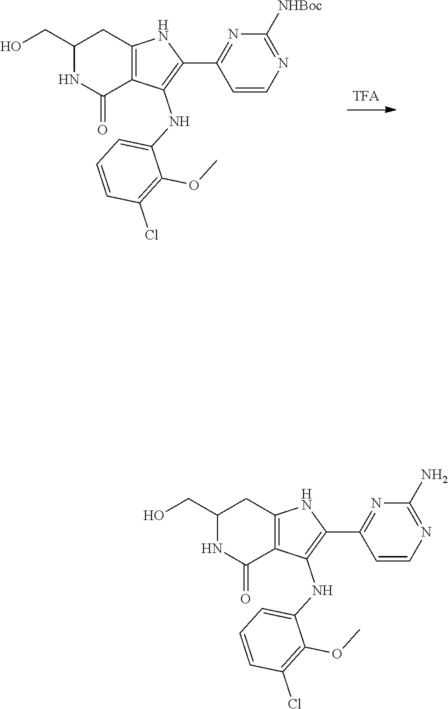 [SMILES: COc1c(Cl)cccc1Nc1c(-c2ccnc(NC(=O)OC(C)(C)C)n2)[nH]c2c1C(=O)NC(CO)C2 ; COc1c(Cl)cccc1Nc1c(-c2ccnc(N)n2)[nH]c2c1C(=O)NC(CO)C2]

Into a 8 mL-sealed tube was placed tert-butyl N-(4-[3-[(3-chloro-2-methoxyphenyl)amino]-6-(hydroxymethyl)-4-oxo-1H,5H,6H,7H-pyrrolo[3,2-c]pyridin-2-yl]pyrimidin-2-yl)carbamate (25.00 mg) in CH₂Cl₂ (1.00 mL), then added TFA (0.30 mL). The resulting mixture was stirred for 2 h at room temperature. The resulting mixture was concentrated under reduced pressure. The crude product was purified by Prep-HPLC with the following conditions (Column: XBridge Shield RP18 OBD Column, 19*250 mm, 10|Ìm; Mobile Phase A: Water (10 mmol/L NH4HCO3+0.1% NH3·H2O), Mobile Phase B: MeOH-HPLC; Flow rate: 25 mL/min; Gradient: 47% B to 72% B in 7 min, 72% B; Wave Length: 254£»220 nm; RT1 (min): 6.4) to afford 2-(2-aminopyrimidin-4-yl)-3-[(3-chloro-2-methoxyphenyl)amino]-6-(hydroxymethyl)-1H,5H,6H,7H-pyrrolo[3,2-c]pyridin-4-one (8.3 mg) as a yellow solid.

LC-MS: M+H found: 415.

¹H NMR (400 MHz, DMSO-d6) δ 11.65 (s, 1H), 8.06 (d, J=5.3 Hz, 1H), 7.92 (s, 1H), 6.89 (d, J=2.0 Hz, 1H), 6.82 (t, J=8.0 Hz, 2H), 6.50 (d, J=5.4 Hz, 1H), 6.36 (dd, J=8.0, 1.7 Hz, 1H), 6.18 (s, 2H), 4.93 (t, J=5.6 Hz, 1H), 3.90 (s, 3H), 3.69-3.56 (m, 1H), 3.55-3.36 (m, 2H), 2.95 (dd, J=16.6, 5.7 Hz, 1H), 2.81 (dd, J=16.6, 8.3 Hz, 1H).

### Example 94. 3′-((3-chloro-2-methoxyphenyl)amino)-2′-(6-methoxy-1,5-naphthyridin-4-yl)-5′,6′-dihydrospiro[cyclobutane-1,7′-pyrrolo[3,2-c]pyridin]-4′(1′H)-one (compound 760)

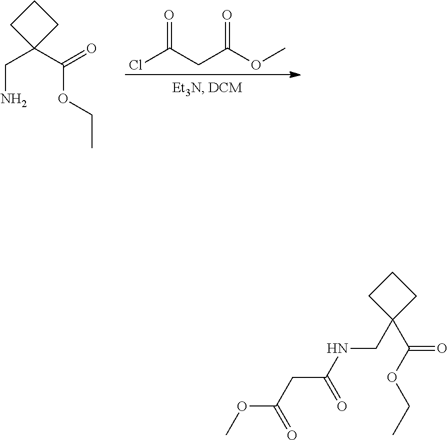

To a mixture of ethyl 1-(aminomethyl)cyclobutane-1-carboxylate (9.00 g, 57.3 mmol, 1.00 equiv) and Et3N (7.00 g, 68.7 mmol, 1.20 equiv) in DCM (90.00 mL) was added methyl 3-chloro-3-oxopropanoate (7.85 g, 57.3 mmol, 1.00 equiv) dropwise at 0 degrees C. The resulting mixture was stirred for additional 1 h at room temperature. The reaction was quenched with Water at room temperature. The resulting mixture was extracted with EtOAc (3×30 mL). The combined organic layers were washed with brine (3×30 mL), dried over anhydrous Na2SO4. After filtration, the filtrate was concentrated under reduced pressure. The residue was purified by silica gel column chromatography, eluted with PE/EtOAc (2:1) to afford ethyl 1-((3-methoxy-3-oxopropanamido)methyl)cyclobutane-1-carboxylate (7.0 g, 50%) as yellow oil.

LC-MS: M+H found: 258.0.

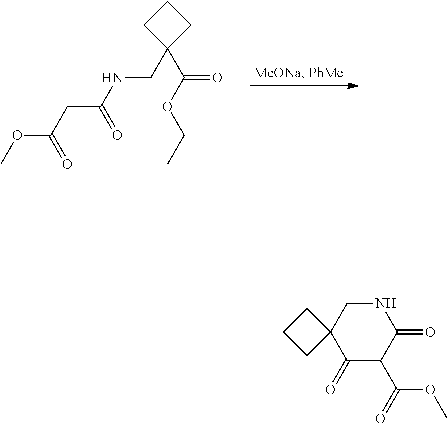 [SMILES: CCOC(=O)C1(CNC(=O)CC(=O)OC)CCC1 ; COC(=O)C1C(=O)NCC2(CCC2)C1=O]

A mixture of ethyl 1-((3-methoxy-3-oxopropanamido)methyl)cyclobutane-1-carboxylate (7.00 g, 27.1 mmol, 1.00 equiv) and MeONa (1.46 g, 27.1 mmol, 1.00 equiv) in PhMe (70.00 mL) was stirred for 3 h at 110 degrees C. The reaction was quenched with Water at room temperature. The mixture was acidified to 20 pH 5 with concentrated HCl. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by reverse flash chromatography (with the following conditions: column, C18 silica gel; mobile phase, MeCN in water, 0% to 10% gradient in 15 min; detector, UV 220 nm) to afford methyl 7,9-dioxo-6-azaspiro[3.5]nonane-8-carboxylate 4 g (69.4%) as an oil.

LC-MS: (M+H)⁺ found: 212.

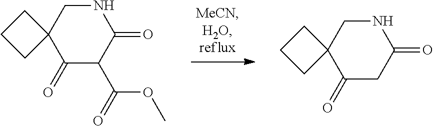

A mixture of methyl 7,9-dioxo-6-azaspiro[3.5]nonane-8-carboxylate (4.00 g, 18.8 mmol, 1.00 equiv) in MeCN (40.00 mL) and Water (4.00 mL) was stirred at 80 degrees C. for overnight. The reaction was quenched with Water at room temperature and concentrated under reduced pressure. The residue was purified by reversed phase flash chromatography (with the following conditions: column, C18 silica gel; mobile phase, MeCN in water, 0% to 20% gradient in 15 min; detector, UV 220 nm) to afford 6-azaspiro[3.5]nonane-7,9-dione 2 g (68.9%) as a white solid.

LC-MS: (M+H)⁺ found: 154.

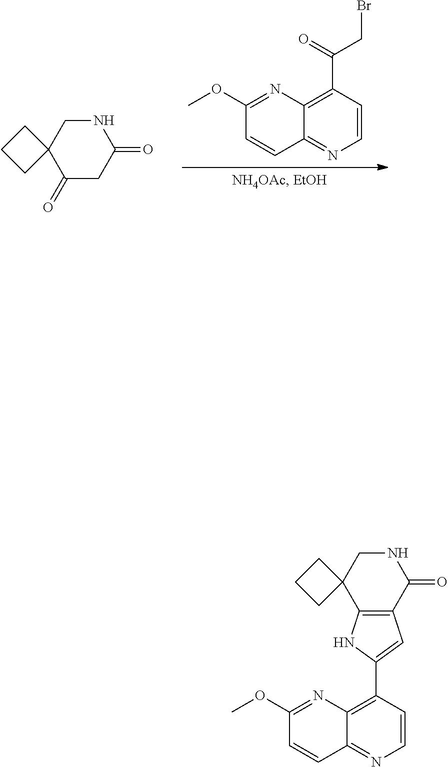 [SMILES: O=C1CC(=O)C2(CCC2)CN1 ; COc1ccc2nccc(C(=O)CBr)c2n1 ; COc1ccc2nccc(-c3cc4c([nH]3)C3(CCC3)CNC4=O)c2n1]

A mixture of 6-azaspiro[3.5]nonane-7,9-dione (300 mg, 1.94 mmol, 1.00 equiv), 2-bromo-1-(6-methoxy-1,5-naphthyridin-4-yl)ethan-1-one (656 mg, 2.33 mmol, 1.20 equiv), and NH₄OAc (900 mg, 11.64 mmol, 6.00 equiv) in EtOH (15.00 mL) was stirred at 50 degrees C. for 3 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (10:1)) to afford 2′-(6-methoxy-1,5-naphthyridin-4-yl)-5′,6′-dihydrospiro[cyclobutane-1,7′-pyrrolo[3,2-c]pyridin]-4′(1′H)-one (360 mg, 55%) as a yellow solid.

LC-MS: (M+H)⁺ found 335.

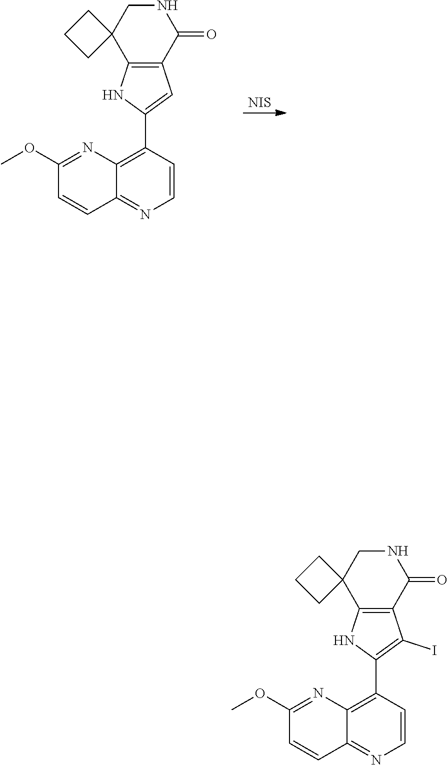 [SMILES: COc1ccc2nccc(-c3cc4c([nH]3)C3(CCC3)CNC4=O)c2n1 ; "NIS" ; COc1ccc2nccc(-c3[nH]c4c(c3I)C(=O)NCC43CCC3)c2n1]

To a stirred mixture of 2′-(6-methoxy-1,5-naphthyridin-4-yl)-5′,6′-dihydrospiro[cyclobutane-1,7′-pyrrolo[3,2-c]pyridin]-4′(1′H)-one (360 mg, 1.07 mmol, 1.00 equiv) in DMF (2.00 mL) was added NIS (186 mg, 1.07 mmol, 1.00 equiv) at 0 degrees C. The mixture was stirred at room temperature for 2 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl2/MeOH (20:1)) to afford 3′-iodo-2′-(6-methoxy-1,5-naphthyridin-4-yl)-5′,6′-dihydrospiro[cyclobutane-1,7′-pyrrolo[3,2-c]pyridin]-4′(1′H)-one (200 mg, 40%) as yellow solid.

LC-MS: (M+H)⁺ found 461.

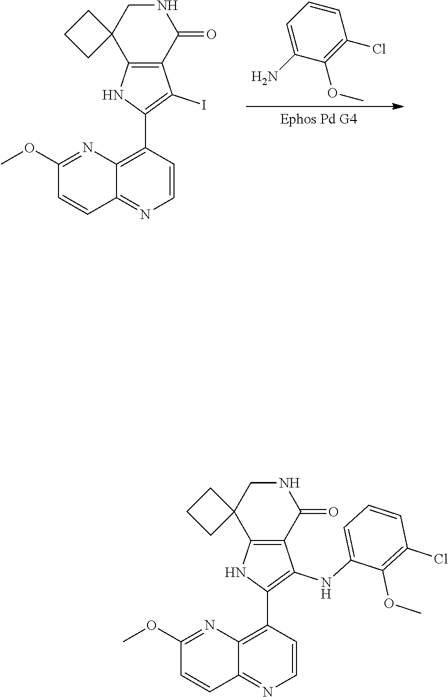

To a stirred solution of 3′-iodo-2′-(6-methoxy-1,5-naphthyridin-4-yl)-5′,6′-dihydrospiro[cyclobutane-1,7′-pyrrolo[3,2-c]pyridin]-4′(1′H)-one (200.00 mg, 0.433 mmol, 1.00 equiv), 3-chloro-2-methoxyaniline (89.1 mg, 0.563 mmol, 1.3 equiv), and Cs₂CO₃ (282 mg, 0.867 mmol, 2.00 equiv) in 1,4-dioxane (5.00 mL) were added EPhos (48.8 mg, 0.086 mmol, 0.20 equiv) and EPhos Pd G4 (41.12 mg, 0.043 mmol, 0.10 equiv) at room temperature under nitrogen atmosphere. The resulting mixture was stirred for 2 h at 50° C. under nitrogen atmosphere. The reaction was quenched with Water at room temperature. The resulting mixture was extracted with EtOAc (3×20 mL). The combined organic layers were washed with brine (2×50 mL), dried over anhydrous Na₂SO₄. After filtration, the filtrate was concentrated under reduced pressure. The residue was purified by Prep-HPLC (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient. 58% B to 65% B in 10 min, 65% B; Wave Length: 220/254 nm; RT1 (min): 9.30) to afford 3′-((3-chloro-2-methoxyphenyl)amino)-2′-(6-methoxy-1,5-naphthyridin-4-yl)-5′,6′-dihydrospiro[cyclobutane-1,7′-pyrrolo[3,2-c]pyridin]-4′(1′H)-one 100 mg crude as a yellow solid.

LC-MS: (M+H)⁺ found: 490.

¹H NMR (400 MHz, DMSO-d₆) δ 12.14-12.02 (m, 1H), 8.64-8.59 (m, 1H), 8.40-8.27 (m, 1H), 7.86-7.77 (m, 1H), 7.58-7.51 (m, 1H), 7.42-7.26 (m, 2H), 6.72-6.60 (m, 2H), 6.20-6.09 (m, 1H), 4.28-4.18 (m, 3H), 3.92-3.82 (m, 3H), 3.56-3.47 (m, 2H), 2.72-2.63 (m, 1H), 2.39-2.27 (m, 1H), 2.14-2.02 (m, 3H), 1.99-1.90 (m, 1H).

### Example 95. (7R)-3-[(3-chloro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (compound 786)

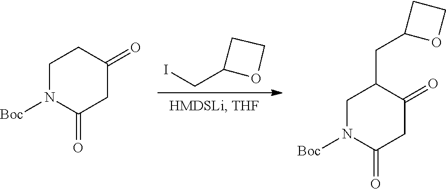

To a stirred solution of tert-butyl 2,4-dioxopiperidine-1-carboxylate (3.59 g, 16.836 mmol, 1.00 equiv) in THF (50 mL) was added LiHMDS (42.09 mL, 42.090 mmol, 2.5 equiv) dropwise at −20 degrees C. under nitrogen atmosphere. To the above mixture was added 2-(iodomethyl)oxetane (5.00 g, 25.254 mmol, 1.5 equiv) dropwise at −20 degrees C. The resulting mixture was stirred for additional 3 h at −20 degrees C. The mixture was acidified to pH 5 with 5% KHSO4 (aq.). The resulting mixture was extracted with EtOAc (3×100 mL). The combined organic layers were washed with brine (2×100 mL), dried over anhydrous Na2SO4. After filtration, the filtrate was concentrated under reduced pressure. The residue was purified by silica gel column chromatography, eluted with PE/EtOAc (2:1) to afford tert-butyl 5-(oxetan-2-ylmethyl)-2,4-dioxopiperidine-1-carboxylate (2.5 g, 52.41%) as a light yellow solid.

LC-MS (M+H)⁺ found: 284.

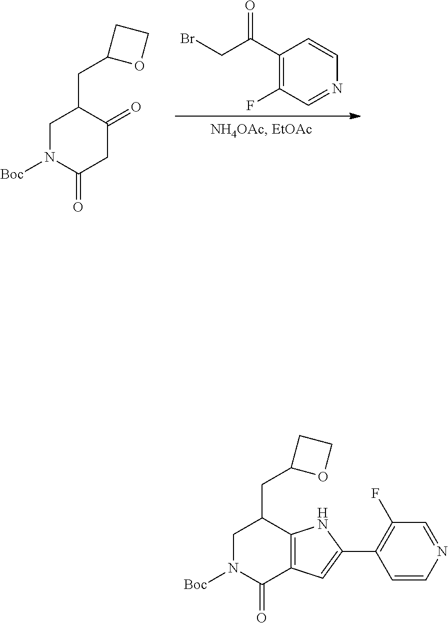

A solution of tert-butyl 5-(oxetan-2-ylmethyl)-2,4-dioxopiperidine-1-carboxylate (2 g, 7.059 mmol, 1.00 equiv) in EtOH (30 mL) was treated with 2-bromo-1-(3-fluoropyridin-4-yl)ethanone (1.85 g, 8.471 mmol, 1.2 equiv) for 2 min at room temperature under nitrogen atmosphere followed by the addition of NH4OAc (3.26 g, 42.354 mmol, 6 equiv) in portions at 60 degrees C. The residue was purified by reverse flash chromatography with the following conditions: column, silica gel; mobile phase, MeCN in water, 10% to 50% gradient in 10 min; detector, UV 220 nm. This resulted in tert-butyl 2-(3-fluoropyridin-4-yl)-7-(oxetan-2-ylmethyl)-4-oxo-1H,6H,7H-pyrrolo[3,2-c]pyridine-5-carboxylate (1 g, 35.29%) as a yellow oil.

LC-MS (M+H)⁺ found: 402.

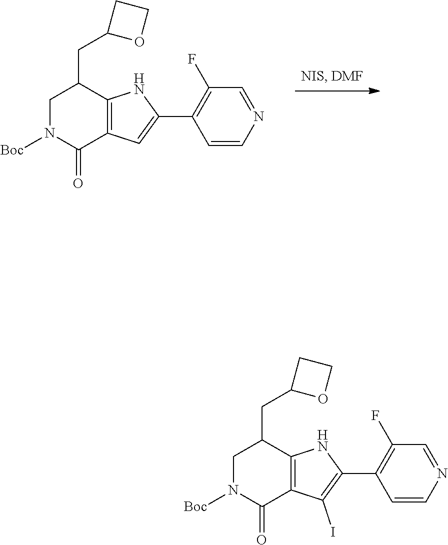 [SMILES: CC(C)(C)OC(=O)N1CC(CC2CCO2)c2[nH]c(-c3ccncc3F)cc2C1=O ; CC(C)(C)OC(=O)N1CC(CC2CCO2)c2[nH]c(-c3ccncc3F)c(I)c2C1=O]

A solution of tert-butyl 2-(3-fluoropyridin-4-yl)-7-(oxetan-2-ylmethyl)-4-oxo-1H,6H,7H-pyrrolo[3,2-c]pyridine-5-carboxylate (1 g, 2.491 mmol, 1.00 equiv) in DMF (15 mL) was treated with NIS (0.56 g, 2.491 mmol, 1 equiv) for 30 min at 0 degrees C. under nitrogen atmosphere. The resulting mixture was stirred for 3 h at room temperature under nitrogen atmosphere. The resulting mixture was extracted with EtOAc (3×50 mL). The combined organic layers were washed with brine (2×30 mL), dried over anhydrous Na2SO4. After filtration, the filtrate was concentrated under reduced pressure. The residue was purified by silica gel column chromatography, eluted with CH2Cl2/MeOH (12:1) to afford tert-butyl 2-(3-fluoropyridin-4-yl)-3-iodo-7-(oxetan-2-ylmethyl)-4-oxo-1H,6H,7H-pyrrolo[3,2-c]pyridine-5-carboxylate (900 mg, 68.51%) as a yellow solid.

LC-MS (M+H)⁺ found: 528.

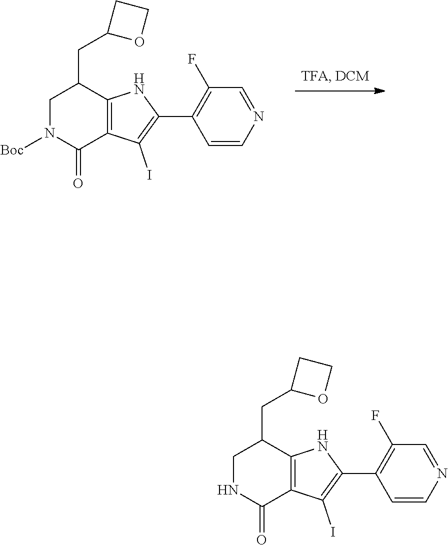 [SMILES: CC(C)(C)OC(=O)N1CC(CC2CCO2)c2[nH]c(-c3ccncc3F)c(I)c2C1=O ; O=C1NCC(CC2CCO2)c2[nH]c(-c3ccncc3F)c(I)c21]

A solution of tert-butyl 2-(3-fluoropyridin-4-yl)-3-iodo-7-(oxetan-2-ylmethyl)-4-oxo-1H,6H,7H-pyrrolo[3,2-c]pyridine-5-carboxylate (900 mg, 1.707 mmol, 1.00 equiv) in DCM (7 mL) was treated with TFA (3 mL) for 2 min at 0 degrees C. under nitrogen atmosphere followed by the addition of TFA (3 mL) dropwise at 0 degrees C. The resulting mixture was stirred for 2 h at room temperature under nitrogen atmosphere. The resulting mixture was extracted with CH₂Cl₂ (3×50 mL). The combined organic layers were washed with brine (2×30 mL), dried over anhydrous Na2SO4. After filtration, the filtrate was concentrated under reduced pressure. The residue was purified by silica gel column chromatography, eluted with CH2Cl2/MeOH (12:1) to afford 2-(3-fluoropyridin-4-yl)-3-iodo-7-(oxetan-2-ylmethyl)-1H,5H,6H,7H-pyrrolo[3,2-c]pyridin-4-one (580 mg, 79.55%) as a yellow solid.

LC-MS (M+H)⁺ found: 428.

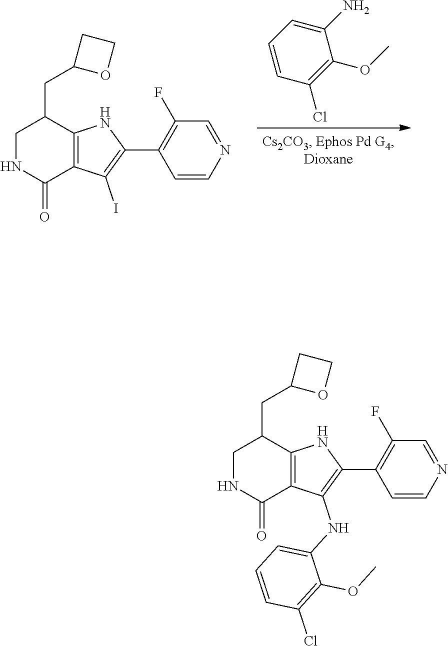

To a stirred mixture of tert-butyl 2-(3-fluoropyridin-4-yl)-3-iodo-7-(oxetan-2-ylmethyl)-4-oxo-1H,6H,7H-pyrrolo[3,2-c]pyridine-5-carboxylate (500 mg, 0.948 mmol, 1.00 equiv) and 3-chloro-2-methoxyaniline (373.58 mg, 2.370 mmol, 2.50 equiv) in dioxane (10 mL) were added Ephos Pd G4 (87.09 mg, 0.095 mmol, 0.10 equiv) and Ephos (101.41 mg, 0.190 mmol, 0.2 equiv) and Cs2CO3 (617.86 mg, 1.896 mmol, 2 equiv) in portions at room temperature under nitrogen atmosphere. The resulting mixture was stirred for 1 h at 50 degrees C. under nitrogen atmosphere. The resulting mixture was concentrated under vacuum. The residue was purified by silica gel column chromatography, eluted with CH₂Cl2/MeOH (10:1) to afford tert-butyl 3-[(3-chloro-2-methoxyphenyl)amino]-2-(3-fluoropyridin-4-yl)-7-(oxetan-2-ylmethyl)-4-oxo-1H,6H,7H-pyrrolo[3,2-c]pyridine-5-carboxylate (300 mg, 56.80%) as a yellow solid.

LC-MS (M+H)⁺ found: 457.

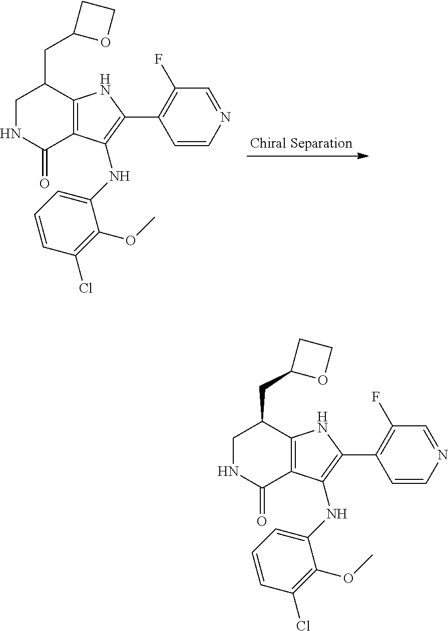 [SMILES: COc1c(Cl)cccc1Nc1c(-c2ccncc2F)[nH]c2c1C(=O)NCC2CC1CCO1 ; COc1c(Cl)cccc1Nc1c(-c2ccncc2F)[nH]c2c1C(=O)NC[C@H]2C[C@H]1CCO1]

The product was separated by Prep-Chiral-HPLC with the following conditions (Column: CHIRAL ART Amylose-SA, 2*25 cm, 5 μm; Mobile Phase A: MtBE (0.5% 2M NH3-MeOH)-HPLC, Mobile Phase B: EtOH-HPLC; Flow rate: 20 mL/min; Gradient: 30% B to 30% B in 18 min; Wave Length: 220/254 nm; RT1 (min): 7.112: RT2 (min): 13.349; Sample Solvent: EtOH-HPLC; Injection Volume: 1.5 mL; Number Of Runs: 4). This resulted in (7R)-3-[(3-chloro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (5.6 mg) as a white solid.

LC-MS: (M+H)⁺ found: 457.00.

¹H NMR (400 MHz, DMSO-d6) δ 11.56 (s, 1H), 8.52 (d, J=3.1 Hz, 1H), 8.29 (d, J=5.2 Hz, 1H), 7.62 (s, 1H), 7.44 (dd, J=6.8, 5.1 Hz, 1H), 7.24 (d, J=2.5 Hz, 1H), 6.74-6.63 (m, 2H), 6.14 (dd, J=7.2, 2.4 Hz, 1H), 5.02-4.91 (m, 1H), 4.62 (td, J=7.9, 5.8 Hz, 1H), 4.52 (dt, J=9.1, 5.8 Hz, 1H), 3.87 (s, 3H), 3.48-3.39 (m, 1H), 3.27-3.07 (m, 2H), 2.81-2.70 (m, 1H), 2.44-2.33 (m, JH), 2.23 (m, 1H), 1.91 (m, 1H).

### Example 96. (7R)-3-[(3-chloro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (compound 785)

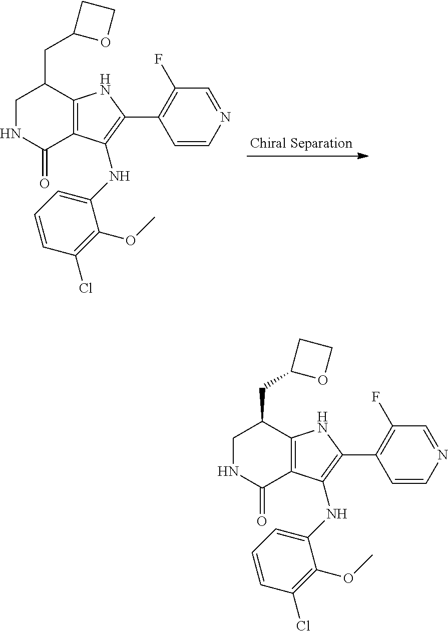 [SMILES: COc1c(Cl)cccc1Nc1c(-c2ccncc2F)[nH]c2c1C(=O)NCC2CC1CCO1 ; COc1c(Cl)cccc1Nc1c(-c2ccncc2F)[nH]c2c1C(=O)NC[C@H]2C[C@@H]1CCO1]

The product was separated by Prep-Chiral-HPLC with the following conditions (Column: CHIRAL ART Amylose-SA, 2*25 cm, 5 μm; Mobile Phase A: MtBE (0.5% 2M NH3-MeOH)-HPLC, Mobile Phase B: EtOH-HPLC; Flow rate: 20 mL/min; Gradient: 30% B to 30% B in 18 min; Wave Length: 220/254 nm; RT1 (min): 7.112; RT2 (min): 13.349; Sample Solvent: EtOH-HPLC; Injection Volume: 1.5 mL; Number Of Runs: 4). This resulted in (7R)-3-[(3-chloro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (23.7 mg) as a yellow solid.

LC-MS: (M+H)⁺ found: 457.00.

¹H NMR (400 MHz, DMSO-d6) δ 11.55 (s, 1H), 8.51 (d, J=2.9 Hz, 1H), 8.30 (dd, J=5. 1, 1.1 Hz, 1H), 7.60 (s, 1H), 7.45 (dd, J=6.8, 5.1 Hz, 1H), 7.22 (s, 1H), 6.72-6.61 (m, 2H), 6.13 (dd, J=7.3, 2.4 Hz, 1H), 4.93 (p, J=6.8 Hz, 1H), 4.55 (td, J=8.0, 5.7 Hz, 1H), 4.42 (dt, J=9.0, 5.7 Hz, 1H), 3.86 (s, 3H), 3.50 (m, 1H), 3.30-3.20 (m, 1H), 3.05 (m, 1H), 2.68-2.58 (m, 1H), 2.33-2.24 (m, 1H), 2.16-1.97 (m, 2H).

### Example 97. (7S)-3-[(3-chloro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (compound 784)

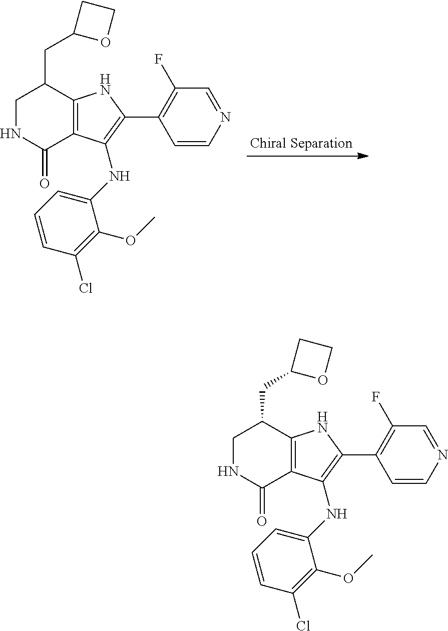 [SMILES: COc1c(Cl)cccc1Nc1c(-c2ccncc2F)[nH]c2c1C(=O)NCC2CC1CCO1 ; COc1c(Cl)cccc1Nc1c(-c2ccncc2F)[nH]c2c1C(=O)NC[C@@H]2C[C@@H]1CCO1]

The product was separated by Prep-Chiral-HPLC with the following conditions (Column: CHIRAL ART Amylose-SA, 2*25 cm, 5 μm; Mobile Phase A: MtBE (0.5% 2M NH3-MeOH)-HPLC, Mobile Phase B: EtOH-HPLC; Flow rate: 20 mL/min; Gradient: 30% B to 30% B in 18 min; Wave Length: 220/254 nm; RT1 (min): 7.112; RT2 (min): 13.349; Sample Solvent: EtOH-HPLC; Injection Volume: 1.5 mL; Number Of Runs: 4). The product was separated by Chiral-Sep. This resulted in (7S)-3-[(3-chloro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (20.7 mg) as a white solid.

LC-MS: (M+H)⁺ found: 457.00.

¹H NMR (400 MHz, DMSO-d6) δ 11.55 (s, 1H), 8.51 (d, J=2.9 Hz, 1H), 8.30 (d, J=5.1 Hz, 1H), 7.60 (s, 1H), 7.45 (dd, J=6.8, 5.1 Hz, 1H), 7.22 (d, J=2.7 Hz, 1H), 6.72-6.61 (m, 2H), 6.13 (dd, J=7.3, 2.4 Hz, 1H), 4.93 (p, J=6.7 Hz, 1H), 4.55 (td, J=8.0, 5.8 Hz, 1H), 4.42 (dt, J=9.1, 5.7 Hz, 1H), 3.86 (s, 3H), 3.50 (ddd, J=12.7, 5.3, 1.9 Hz, 1H), 3.25 (ddd, J=12.6, 5.6, 3.5 Hz, 1H), 3.05 (dd, J=7.7, 5.2 Hz, 1H), 2.68-2.58 (m, 1H), 2.30 (dd, J=13.5, 6.2 Hz, 1H), 2.07 (ddt, J=26.5, 13.9, 6.4 Hz, 2H).

### Example 98. (7S)-3-[(3-chloro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (compound 783)

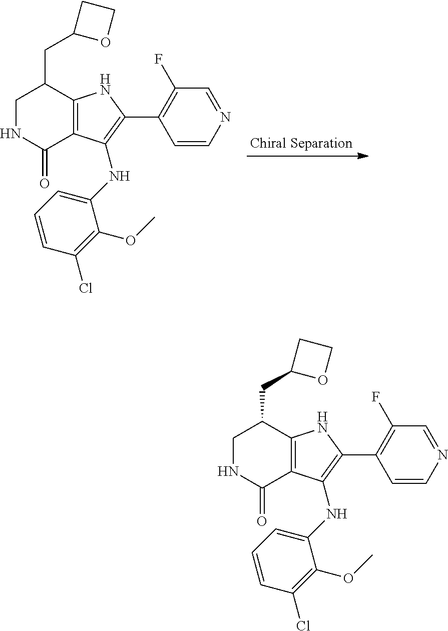 [SMILES: COc1c(Cl)cccc1Nc1c(-c2ccncc2F)[nH]c2c1C(=O)NCC2CC1CCO1 ; COc1c(Cl)cccc1Nc1c(-c2ccncc2F)[nH]c2c1C(=O)NC[C@@H]2C[C@H]1CCO1]

The product was separated by Prep-Chiral-HPLC with the following conditions (Column: CHIRAL ART Amylose-SA, 2*25 cm, 5 sm; Mobile Phase A: MtBE (0.5% 2M NH3-MeOH)-HPLC, Mobile Phase B: EtOH-HPLC; Flow rate: 20 mL/min; Gradient: 30% B to 30% B in 18 min; Wave Length: 220/254 nm: RT1 (min): 7.112; RT2 (min): 13.349; Sample Solvent: EtOH-HPLC; Injection Volume: 1.5 mL; Number Of Runs: 4). This resulted in (7S)-3-[(3-chloro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (6.1 mg) as a white solid.

LC-MS: (M+H)⁺ found: 457.00.

¹H NMR (400 MHz, DMSO-d6) δ 11.56 (s, 1H), 8.52 (d, J=3.0 Hz, 1H), 8.29 (d, J=5.2 Hz, 1H), 7.62 (s, 1H), 7.44 (dd, J=6.9, 5.1 Hz, 1H), 7.24 (d, J=2.6 Hz, 1H), 6.74-6.63 (m, 2H), 6.14 (dd, J=7.2, 2.4 Hz, 1H), 4.96 (m, 1H), 4.62 (td, J=8.0, 5.8 Hz, 1H), 4.52 (dt, J=9.0, 5.8 Hz, 1H), 3.88 (s, 3H), 3.44 (m, 1H), 3.27-3.07 (m, 2H), 2.81-2.69 (m, 1H), 2.44-2.33 (m, 1H), 2.23 (m, 1H), 1.91 (m, 1H).

### Example 99. (7R)-3-[(3-fluoro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (compound 422a)

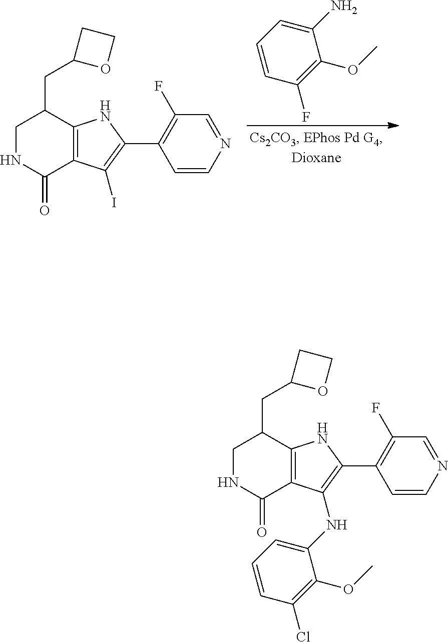

To a stirred mixture of tert-butyl 2-(3-fluoropyridin-4-yl)-3-iodo-7-(oxetan-2-ylmethyl)-4-oxo-1H,6H,7H-pyrrolo[3,2-c]pyridine-5-carboxylate (500 mg, 0.948 mmol, 1.00 equiv) and 3-fluoro-2-methoxyaniline (330.38 mg, 2.340 mmol, 2 equiv) in dioxane (10 mL) were added Ephos Pd G4 (87.09 mg, 0.095 mmol, 0.10 equiv) and Ephos (101.41 mg, 0.190 mmol, 0.2 equiv) and Cs2CO3 (617.86 mg, 1.896 mmol, 2 equiv) in portions at room temperature under nitrogen atmosphere. The resulting mixture was stirred for 1 h at 50 degrees C. under nitrogen atmosphere. The resulting mixture was concentrated under vacuum. The residue was purified by silica gel column chromatography, eluted with CH2Cl2/MeOH (10:1) to afford 3-[(3-fluoro-2-methoxyphenyl)amino]-2-(3-fluoropyridin-4-yl)-7-(oxetan-2-ylmethyl)-1H,5H,6H,7H-pyrrolo[3,2-c]pyridin-4-one (300 mg, 58.20%) as a yellow solid.

LC-MS (M+H)⁺ found: 441.

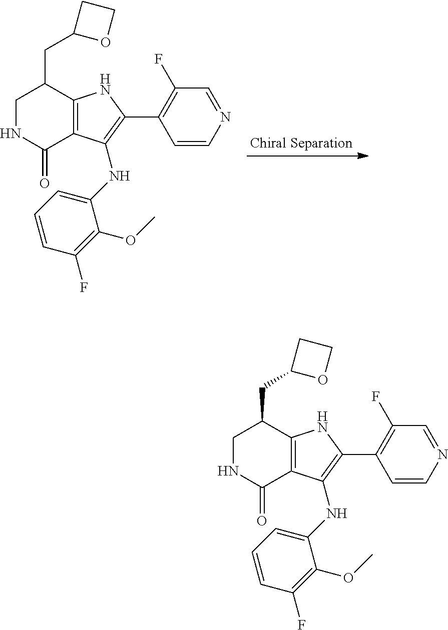 [SMILES: COc1c(F)cccc1Nc1c(-c2ccncc2F)[nH]c2c1C(=O)NCC2CC1CCO1 ; COc1c(F)cccc1Nc1c(-c2ccncc2F)[nH]c2c1C(=O)NC[C@H]2C[C@@H]1CCO1]

3-[(3-fluoro-2-methoxyphenyl)amino]-2-(3-fluoropyridin-4-yl)-7-(oxetan-2-ylmethyl)-1H,5H,6H,7H-pyrrolo[3,2-c]pyridin-4-one (300 mg) was separated by Prep-Chiral-HPLC with the following conditions (Column. CHIRALPAK IA-3, 4.6*50 mm, 3 um; Mobile Phase A: MtBE (0.1% DEA):EtOH=70:30; Flow rate: 1 mL/min; Gradient: 0% B to 0% B; Injection Volume: 5 ul mL). This resulted in (7R)-3-[(3-fluoro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (1.3 mg) as a white solid.

LC-MS: (M+H)⁺ found: 440.95.

¹H NMR (300 MHz, Chloroform-d) δ 11.45 (s, 1H), 8.44 (d, J=4.4 Hz, 1H), 8.13 (d, J=5.6 Hz, 1H), 7.74 (s, 1H), 7.44 (t, J=6.4 Hz, 1H), 6.71-6.49 (m, 2H), 6.08 (d, J=8.0 Hz, 1H), 5.30 (s, 1H), 5.20-5.08 (m, 1H), 5.00-4.78 (m, 2H), 4.13 (d, J=1.4 Hz, 3H), 3.49-3.39 (m, 2H), 3.27 (q, J=9.7 Hz, 1H), 2.97 (s, 1H), 2.53 (s, 1H), 2.37 (m, 1H), 1.83 (d, J=14.4 Hz, 1H).

### Example 100. (7R)-3-[(3-fluoro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (compound 422d)

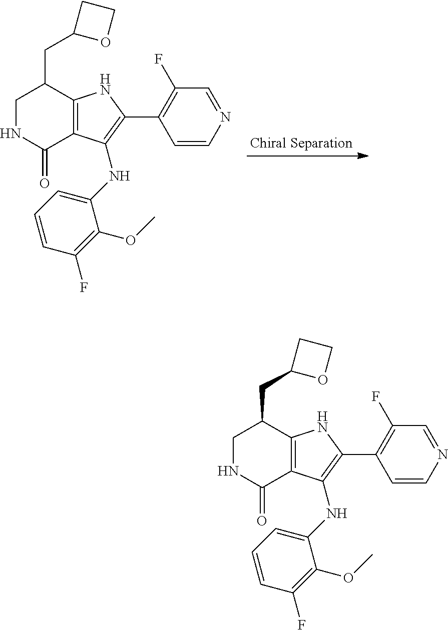 [SMILES: COc1c(F)cccc1Nc1c(-c2ccncc2F)[nH]c2c1C(=O)NCC2CC1CCO1 ; COc1c(F)cccc1Nc1c(-c2ccncc2F)[nH]c2c1C(=O)NC[C@H]2C[C@H]1CCO1]

3-[(3-fluoro-2-methoxyphenyl)amino]-2-(3-fluoropyridin-4-yl)-7-(oxetan-2-ylmethyl)-1H,5H,6H,7H-pyrrolo[3,2-c]pyridin-4-one (300 mg) was separated by Prep-Chiral-HPLC with the following conditions (Column: CHIRALPAK IA-3, 4.6*50 mm, 3 um; Mobile Phase A: MtBE (0.1% DEA):EtOH=70:30; Flow rate: 1 mL/min; Gradient: 0% B to 0% B; Injection Volume: 5 ul mL). This resulted in (7R)-3-[(3-fluoro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (5 mg) as a light yellow solid.

LC-MS: (M+H)⁺ found: 440.95.

¹H NMR (300 MHz, Chloroform-d) δ 10.97 (s, 1H), 8.45 (d, J=4.3 Hz, 1H), 8.15 (d, J=5.4 Hz, 1H), 7.69 (s, 1H), 7.49-7.38 (m, 1H), 6.72-6.49 (m, 2H), 6.10 (d, J=8.1 Hz, 1H), 5.33-5.20 (m, 2H), 4.89 (q, J=7.7 Hz, 1H), 4.61 (m, 1H), 4.14 (d, J=1.4 Hz, 3H), 3.75-3.40 (m, 3H), 2.85-2.61 (m, 2H), 2.23 (m, 1H), 1.90-1.78 (m, 1H).

### Example 101. (7S)-3-[(3-fluoro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (compound 422b)

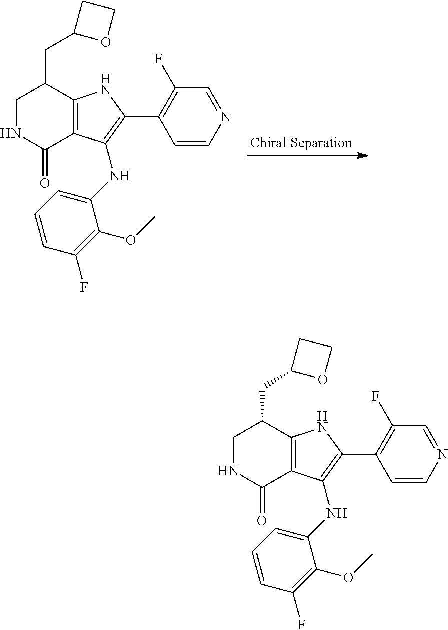 [SMILES: COc1c(F)cccc1Nc1c(-c2ccncc2F)[nH]c2c1C(=O)NCC2CC1CCO1 ; COc1c(F)cccc1Nc1c(-c2ccncc2F)[nH]c2c1C(=O)NC[C@@H]2C[C@@H]1CCO1]

3-[(3-fluoro-2-methoxyphenyl)amino]-2-(3-fluoropyridin-4-yl)-7-(oxetan-2-ylmethyl)-1H,5H,6H,7H-pyrrolo[3,2-c]pyridin-4-one (300 mg) was separated by Prep-Chiral-HPLC with the following conditions (Column: CHIRALPAK IA-3, 4.6*50 mm, 3 um; Mobile Phase A: MtBE (0.1% DEA): EtOH=70: 30; Flow rate: 1 mL/min; Gradient: 0% B to 0% B; Injection Volume: 5 ul mL). This resulted in (7S)-3-[(3-fluoro-2-methoxyphenyl)amino]-2-(3-fluoropyridin-4-yl)-7-[(2R)-oxetan-2-ylmethyl]-1H,5H,6H,7H-pyrrolo[3,2-c]pyridin-4-one (4.4 mg) as a white solid.

LC-MS: (M+H)⁺ found: 440.95.

¹H NMR (300 MHz, Chloroform-d) δ 11.44 (s, 1H), 8.44 (d, J=4.3 Hz, 1H), 8.13 (d, J=5.5 Hz, 1H), 7.73 (s, 1H), 7.44 (t, J=6.4 Hz, 1H), 6.71-6.58 (m, 1H), 6.55 (t, J=9.6 Hz, 1H), 6.09 (d, J=8.1 Hz, 1H), 5.30 (s, 1H), 5.23-5.11 (m, 1H), 5.00-4.78 (m, 2H), 4.13 (d, J=1.4 Hz, 3H), 3.44 (d, J=10.6 Hz, 2H), 3.35-3.23 (m, 1H), 2.97 (s, 1H), 2.58-2.46 (m, 1H), 2.36 (dt, J=14.8, 10.6 Hz, 1H), 1.83 (d, J=14.5 Hz, 1H).

### Example 102. (7S)-2-(2-aminopyrimidin-4-yl)-3-[(3-chloro-2-methoxyphenyl)amino]-7-[(2R)-1,4-dioxan-2-ylmethyl]-1H,5H,6H,7H-pyrrolo[3,2-c]pyridin-4-one (compound 532)

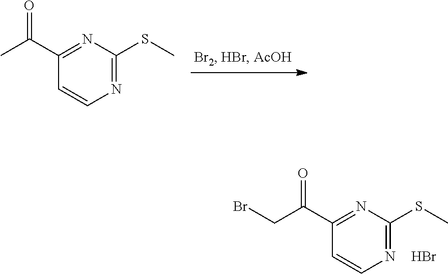

To a stirred mixture of 1-[2-(methylsulfanyl)pyrimidin-4-yl]ethanone (20 g, 118.899 mmol, 1 equiv.) and HBr (20.00 mL, 684.858 mmol, 5.76 equiv.) in AcOH (60 mL) was added Br2 (24.70 g, 154.569 mmol, 1.3 equiv.) at room temperature under nitrogen atmosphere. The resulting mixture was stirred for 3 h at 60° C. Desired product could be detected by LCMS. The precipitated solids were collected by filtration and washed with ethyl acetate (3×20 mL). This resulted in 2-bromo-1-[2-(methylsulfanyl)pyrimidin-4-yl]ethanone hydrobromide (28 g, 71.79%) as a yellow solid.

LC-MS: (M+H)⁺ found 246.95.

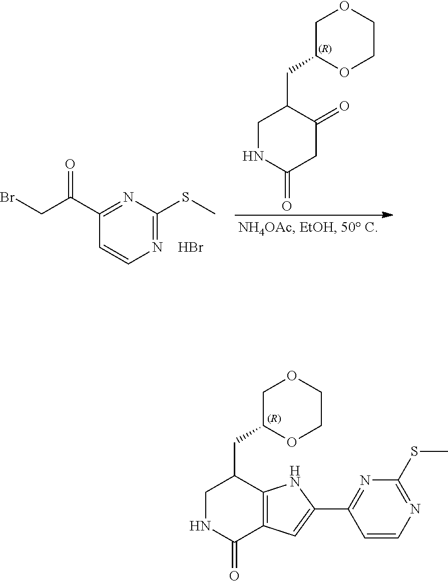

To a stirred solution of 5-[(2R)-1,4-dioxan-2-ylmethyl]piperidine-2,4-dione (3.2 g, 15.007 mmol, 1 equiv.) in EtOH (30 mL) were added 2-bromo-1-[2-(methylsulfanyl)pyrimidin-4-yl]ethanone hydrobromide (5.91 g, 18.008 mmol, 1.2 equiv.) and NH₄OAc (11.57 g, 150.070 mmol, 10 equiv.) at room temperature under nitrogen atmosphere. The resulting mixture was stirred for 2 h at 60° C. Desired product could be detected by LCMS. The aqueous layer was extracted with CH₂Cl₂ (3×100 mL). The resulting mixture was concentrated under reduced pressure. The residue was purified by silica gel column chromatography, eluted with CH₂Cl₂/MeOH (25:1) to afford 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 36.98%) as a brown solid.

LC-MS: (M+H)⁺ found: 361.30.

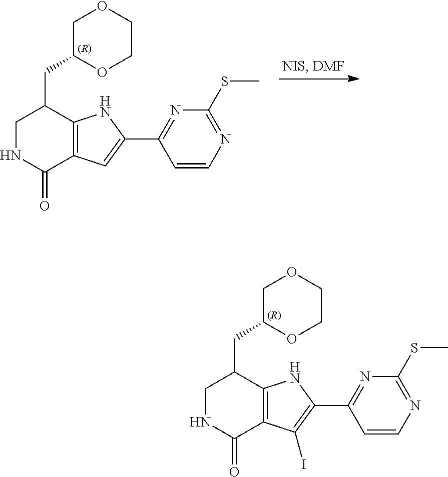

To a stirred solution of 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 5.549 mmol, 1 equiv.) in DMF (20 mL) was added NIS (1.50 g, 6.659 mmol, 1.2 equiv.) at 0° C. under nitrogen atmosphere. The resulting mixture was stirred for overnight at room temperature. Desired product could be detected by LCMS. The aqueous layer was extracted with EtOAc (3×20 mL). The resulting mixture was washed with 2×50 mL of water. The residue was purified by silica gel column chromatography, eluted with CH₂Cl₂/MeOH (30:1) to afford 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.65 g, 61.14%) as a light yellow solid.

LC-MS: (M+H)⁺ found: 487.15.

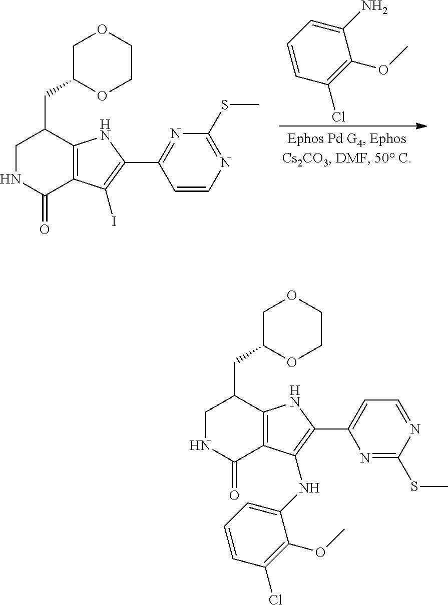

To a stirred mixture of 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.5 g, 3.084 mmol, 1 equiv.) and 3-chloro-2-methoxyaniline (1.46 g, 9.252 mmol, 3 equiv.) in DMF (20 mL) were added Cs₂CO₃ (3.01 g, 9.252 mmol, 3 equiv.) and Ephos Pd G4 (0.28 g, 0.308 mmol, 0.1 equiv.) at room temperature under nitrogen atmosphere. The resulting mixture was stirred for 1 h at 50° C. Desired product could be detected by LCMS. The residue was purified by reverse flash chromatography with the following conditions: column, silica gel; mobile phase, MeCN in water, 40% to 60% gradient in 10 min; detector, UV 254 nm. This resulted in 3-[(3-chloro-2-methoxyphenyl)amino]-7-[(2R)-1,4-dioxan-2-ylmethyl]-2-[2-(methylsulfanyl)pyrimidin-4-yl]-1H,5H,6H,7H-pyrrolo[3,2-c]pyridin-4-one (1 g, 62.83%) as a yellow solid.

LC-MS: (M+H)⁺ found 516.30.

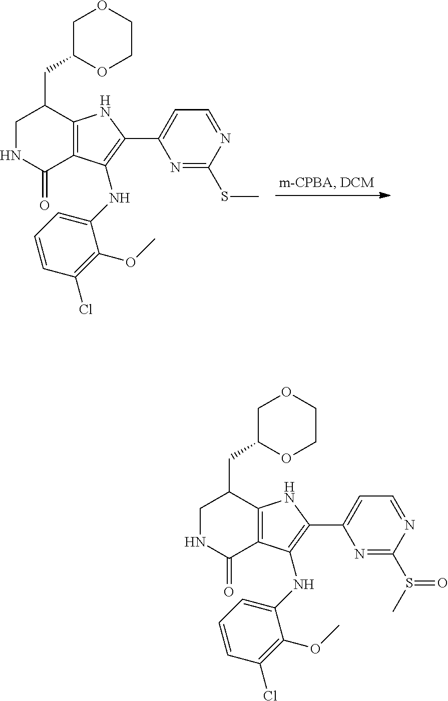 [SMILES: COc1c(Cl)cccc1Nc1c(-c2ccnc(SC)n2)[nH]c2c1C(=O)NCC2C[C@@H]1COCCO1 ; COc1c(Cl)cccc1Nc1c(-c2ccnc(S(C)=O)n2)[nH]c2c1C(=O)NCC2C[C@@H]1COCCO1]

To a stirred solution of 3-[(3-chloro-2-methoxyphenyl)amino]-7-[(2R)-1,4-dioxan-2-ylmethyl]-2-[2-(methylsulfanyl)pyrimidin-4-yl]-1H,5H,6H,7H-pyrrolo[3,2-c]pyridin-4-one (350 mg, 0.678 mmol, 1 equiv.) in DCM (5 mL) was added m-CPBA (234.10 mg, 1.356 mmol, 2 equiv.) at room temperature under nitrogen atmosphere. The resulting mixture was stirred for 10 min at room temperature. Desired product could be detected by LCMS. The aqueous layer was extracted with EtOAc (3×10 mL). The resulting mixture was washed with 3×10 mL of sat.NaHCO₃. The resulting mixture was concentrated under reduced pressure. This resulted in 3-[(3-chloro-2-methoxyphenyl)amino]-7-[(2R)-1,4-dioxan-2-ylmethyl]-2-(2-methanesulfinylpyrimidin-4-yl)-1H,5H,6H,7H-pyrrolo[3,2-c]pyridin-4-one (370 mg, 92.28%) as a reddish solid.

LC-MS: (M+H)⁺ found: 532.45.

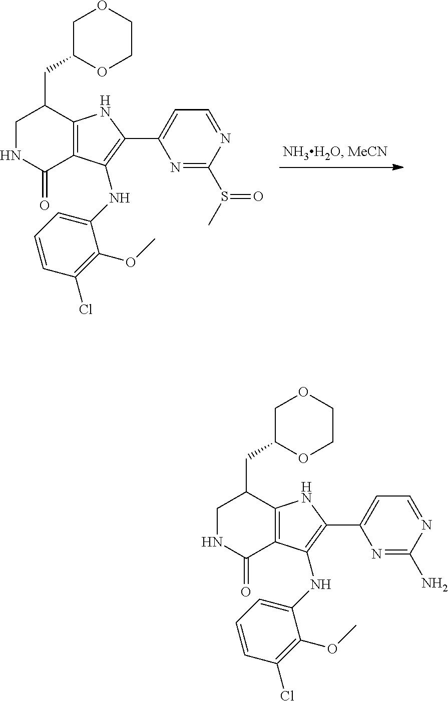 [SMILES: COc1c(Cl)cccc1Nc1c(-c2ccnc(S(C)=O)n2)[nH]c2c1C(=O)NCC2C[C@@H]1COCCO1 ; COc1c(Cl)cccc1Nc1c(-c2ccnc(N)n2)[nH]c2c1C(=O)NCC2C[C@@H]1COCCO1]

To a stirred solution of 3-[(3-chloro-2-methoxyphenyl)amino]-7-[(2R)-1,4-dioxan-2-ylmethyl]-2-(2-methanesulfinylpyrimidin-4-yl)-1H,5H,6H,7H-pyrrolo[3,2-c]pyridin-4-one (370 mg, 0.626 mmol, 1 equiv, 90%) in ACN (4 mL) was added NH3·H2O (2 mL, 15.408 mmol, 24.62 equiv, 30%) at room temperature under nitrogen atmosphere. The resulting mixture was stirred for overnight at 80° C. Desired product could be detected by LCMS. The resulting mixture was concentrated under reduced pressure. This resulted in 150 mg crude product.

LC-MS: (M+H)⁺ found: 485.2.

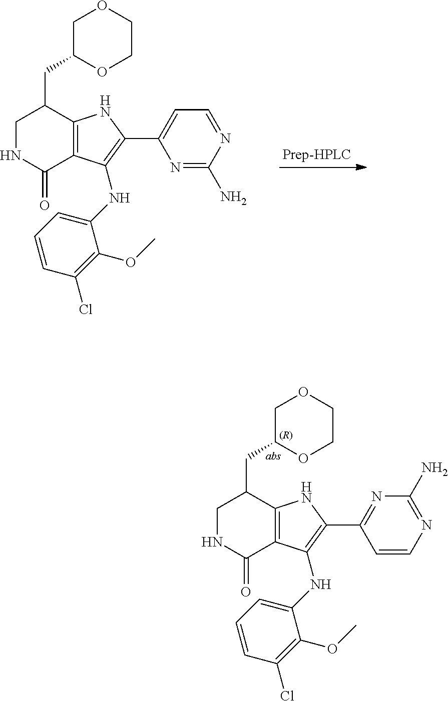

The crude product (150 mg) was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 25% B to 35% B in 10 min, 35% B; Wave Length: 254/220 nm; RT1 (min): 9.92 to afford (7S)-2-(2-aminopyrimidin-4-yl)-3-[(3-chloro-2-methoxyphenyl)amino]-7-[(2R)-1,4-dioxan-2-ylmethyl]-1H,5H,6H,7H-pyrrolo[3,2-c]pyridin-4-one (19.6 mg, 12.81%) as a yellow solid.

LC-MS: (M+H)⁺ found: 485.2.

¹H NMR (300 MHz, Chloroform-d) δ 10.17 (s, 1H), 8.01 (d, J=5.7 Hz, 1H), 7.65 (s, 1H), 6.87-6.67 (m, 2H), 6.49-6.32 (m, 2H), 5.35 (d, J=12.7 Hz, 3H), 4.05 (s, 3H), 3.97-3.54 (m, 7H), 3.45-3.24 (m, 3H), 2.00 (s, 1H), 1.69 (ddd, J=13.8, 8.9, 4.8 Hz, 1H).

### Example 103. (7S)-7-[(2R)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (compound 529)

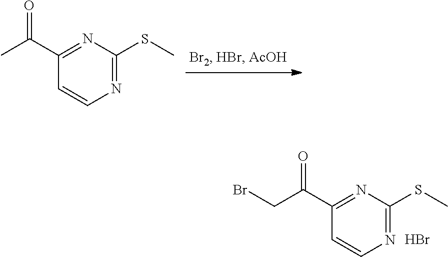

To a stirred mixture of 1-[2-(methylsulfanyl)pyrimidin-4-yl]ethanone (20 g, 118.899 mmol, 1 equiv.) and HBr (20.00 mL, 684.858 mmol, 5.76 equiv.) in AcOH (60 mL) was added Br2 (24.70 g, 154.569 mmol, 1.3 equiv.) at room temperature under nitrogen atmosphere. The resulting mixture was stirred for 3 h at 60° C. Desired product could be detected by LCMS. The precipitated solids were collected by filtration and washed with ethyl acetate (3×20 mL). This resulted in 2-bromo-1-[2-(methylsulfanyl)pyrimidin-4-yl]ethanone hydrobromide (28 g, 71.79%) as a yellow solid.

LC-MS: (M+H)⁺ found: 246.95.

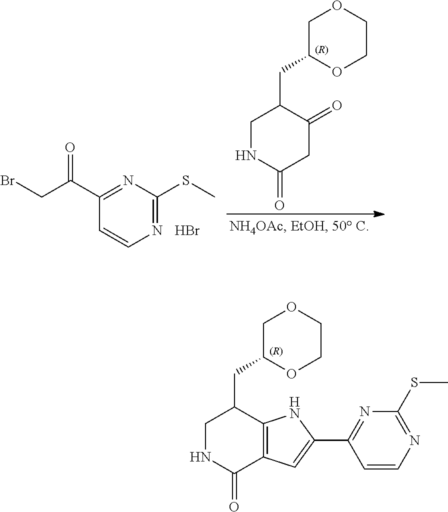

To a stirred solution of 5-[(2R)-1,4-dioxan-2-ylmethyl]piperidine-2,4-dione (3.2 g, 15.007 mmol, 1 equiv.) in EtOH (30 mL) were added 2-bromo-1-[2-(methylsulfanyl)pyrimidin-4-yl]ethanone hydrobromide (5.91 g, 18.008 mmol, 1.2 equiv.) and NH₄OAc (11.57 g, 150.070 mmol, 10 equiv.) at room temperature under nitrogen atmosphere. The resulting mixture was stirred for 2 h at 60° C. Desired product could be detected by LCMS. The aqueous layer was extracted with CH₂Cl₂ (3×100 mL). The resulting mixture was concentrated under reduced pressure. The residue was purified by silica gel column chromatography, eluted with CH₂Cl₂/MeOH (25:1) to afford 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 36.98%) as a brown solid.

LC-MS: (M+H)⁺ found: 361.30.

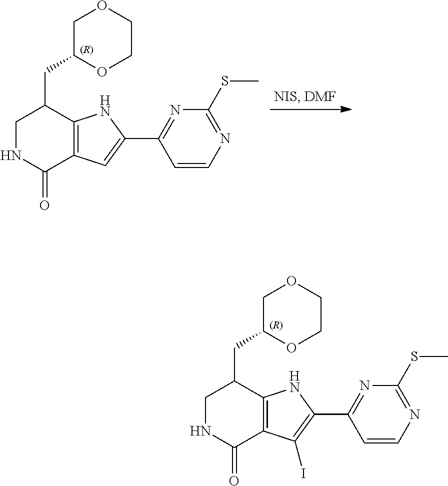

To a stirred solution of 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 5.549 mmol, 1 equiv.) in DMF (20 mL) was added NIS (1.50 g, 6.659 mmol, 1.2 equiv.) at 0° C. under nitrogen atmosphere. The resulting mixture was stirred for overnight at room temperature. Desired product could be detected by LCMS. The aqueous layer was extracted with EtOAc (3×20 mL). The resulting mixture was washed with 2×50 mL of water. The residue was purified by silica gel column chromatography, eluted with CH₂Cl₂/MeOH (30:1) to afford 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.65 g, 61.14%) as a light yellow solid.

LC-MS: (M+H)⁺ found: 487.15.

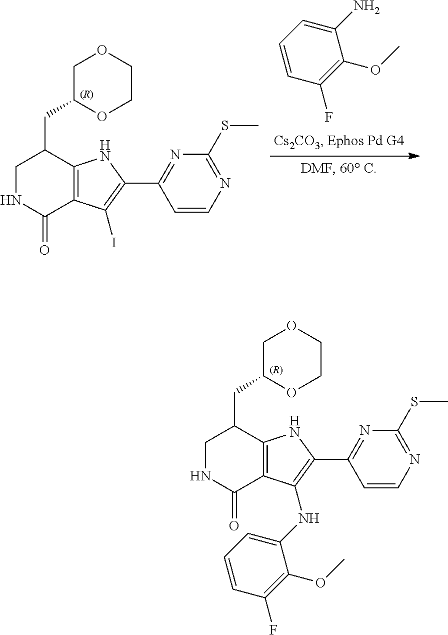

To a stirred mixture of 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.1 g, 2.262 mmol, 1 equiv.) and 3-fluoro-2-methoxyaniline (0.96 g, 6.786 mmol, 3 equiv.) in DMF (15 mL) were added Cs₂CO₃ (2.21 g, 6.786 mmol, 3 equiv.) and Ephos Pd G4 (0.21 g, 0.226 mmol, 0.1 equiv.) at room temperature under nitrogen atmosphere. The resulting mixture was stirred for 1 h at 50° C. Desired product could be detected by LCMS. The residue was purified by reverse flash chromatography with the following conditions: column, silica gel; mobile phase, MeCN in water, 45% to 70% gradient in 10 min; detector, UV 254 nm. This resulted in 7-[(2R)-1,4-dioxan-2-ylmethyl]-3-[(3-fluoro-2-methoxyphenyl)amino]-2-[2-(methylsulfanyl)pyrimidin-4-yl]-1H,5H,6H,7H-pyrrolo[3,2-c]pyridin-4-one (810 mg, 71.69%) as a yellow solid.

LC-MS: (M+H)⁺ found: 500.10.

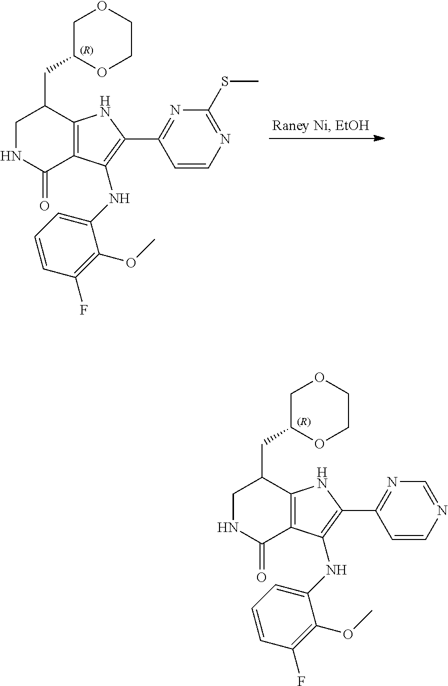

To a stirred solution of (7S)-7-[(2R)-1,4-dioxan-2-ylmethyl]-3-[(3-fluoro-2-methoxyphenyl)amino]-2-[2-(methylsulfanyl)pyrimidin-4-yl]-1H,5H,6H,7H-pyrrolo[3,2-c]pyridin-4-one, 1 equiv.) in EtOH (30 mL) was added Raney-Ni (2.78 g, 32.420 mmol, 20 equiv.) at room temperature under nitrogen atmosphere. The resulting mixture was stirred for 24 h at 90° C. Desired product could be detected by LCMS. The resulting mixture was filtered, the filter cake was washed with EtOH (3×30 mL). The filtrate was concentrated under reduced pressure. This resulted in 7-[(2R)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (600 mg, 81.59%) as a yellow solid.

LC-MS: (M+H)⁺ found: 454.15.

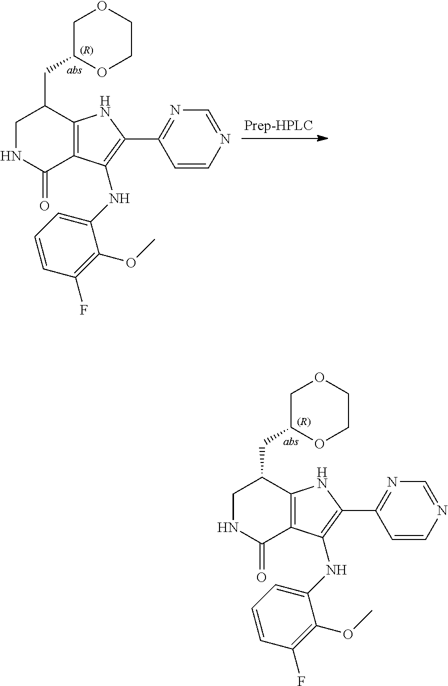

The crude product (600 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 23% B to 37% B in 10 min, 37% B: Wave Length: 254/220 nm; RT1 (min): 7 10) to afford (7S)-7-[(2R)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (92.4 mg, 15.37%) as a yellow solid.

LC-MS: (M+H)⁺ found: 454.10.

¹H NMR (300 MHz, Chloroform-d) δ 10.35 (s, 1H), 8.75 (d, J=177.8 Hz, 2H), 7.53 (s, 1H), 7.02 (s, 1H), 6.71 (d, J=7.1 Hz, 1H), 6.61-6.45 (m, 1H), 6.19 (d, J=7.7 Hz, 1H), 5.52 (s, 1H), 4.10 (d, J=1.3 Hz, 3H), 4.03 (d, J=11.2 Hz, 1H), 3.74 (ddt, J=44.3, 34.9, 10.1 Hz, 6H), 3.49-3.14 (m, 3H), 1.98 (t, J=12.0 Hz, 1H), 1.74-1.64 (m, JH).

### Example 104. (7S)-3-[(3-chloro-2-methoxyphenyl)amino]-7-[(2R)-1,4-dioxan-2-ylmethyl]-2-(pyrimidin-4-yl)-1H,5H,6H,7H-pyrrolo[3,2-c]pyridin-4-one (compound 528)

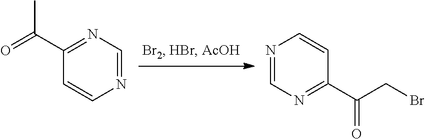

To a stirred solution of 1-(pyrimidin-4-yl)ethanone (1 g, 8.188 mmol, 1.00 equiv) and HBr (1.45 mL, 49.640 mmol, 6.06 equiv) in AcOH (3 mL) was added Br2 (1 g, 6.258 mmol, 0.76 equiv) dropwise at room temperature. Then the mixture was stirred at 60 degree for 3 h. After the reaction was completed, the mixture was diluted with ethyl acetate. The solution was stirred for overnight. The solid was filtrated and dried to get 2-bromo-1-(pyrimidin-4-yl)ethanone (1.3 g, 78.98%) brown solid as product.

LC-MS: (M+H)⁺ found: 200.95.

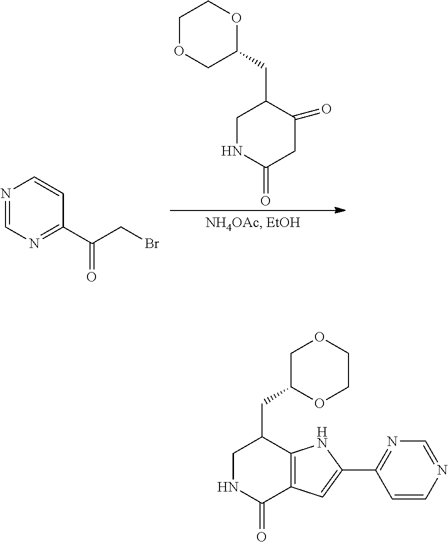 [SMILES: O=C(CBr)c1ccncn1 ; O=C1CC(=O)C(C[C@@H]2COCCO2)CN1 ; O=C1NCC(C[C@@H]2COCCO2)c2[nH]c(-c3ccncn3)cc21]

A solution of 2-bromo-1-(pyrimidin-4-yl)ethanone hydrobromide (3 g, 10.641 mmol, 1 equiv) in EtOH (50 mL) was treated with 5-[(2R)-1,4-dioxan-2-ylmethyl]piperidine-2,4-dione (2.27 g, 10.641 mmol, 1 equiv) for 2 min at room temperature under nitrogen atmosphere followed by the addition of NH4OAc (8.20 g, 106.410 mmol, 10 equiv) in portions at room temperature. The resulting mixture was stirred for 2 h at 60° C. under nitrogen atmosphere. The resulting mixture was extracted with EtOAc (8×100 mL). The combined organic layers were dried over anhydrous Na2SO4. After filtration, the filtrate was concentrated under reduced pressure. The residue was purified by silica gel column chromatography, eluted with CH2Cl2/MeOH (10:1) to afford 7-[(2R)-1,4-dioxan-2-ylmethyl]-2-(pyrimidin-4-yl)-1H,5H,6H,7H-pyrrolo[3,2-c]pyridin-4-one (1.5 g, 44.84%) as a yellow solid.

LC-MS: (M+H)⁺ found: 315.10.

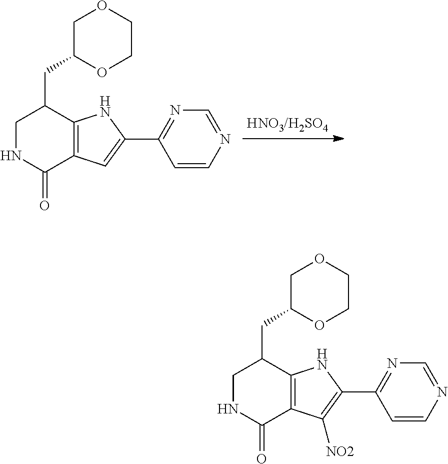 [SMILES: O=C1NCC(C[C@@H]2COCCO2)c2[nH]c(-c3ccncn3)cc21 ; O=C1NCC(C[C@@H]2COCCO2)c2[nH]c(-c3ccncn3)c([N+](=O)[O-])c21]

A solution of 7-[(2R)-1,4-dioxan-2-ylmethyl]-2-(pyrimidin-4-yl)-1H,5H,6H,7H-pyrrolo[3,2-c]pyridin-4-one (700 mg, 2.227 mmol, 1 equiv) in H2SO4 (10 mL) was treated with Nitric acid fuming (140.32 mg, 2.227 mmol, 1 equiv) for 2 min at −5° C. under nitrogen atmosphere. The resulting mixture was stirred for 1 h at 0° C. under nitrogen atmosphere. The resulting mixture was diluted with water (40 mL). The mixture was basified to pH 9 with NaOH (aq.). The resulting mixture was extracted with CH₂Cl₂ (5×50 mL). The combined organic layers were dried over anhydrous Na2SO4. After filtration, the filtrate was concentrated under reduced pressure. This resulted in 7-[(2R)-1,4-dioxan-2-ylmethyl]-3-nitro-2-(pyrimidin-4-yl)-1H,5H,6H,7H-pyrrolo[3,2-c]pyridin-4-one (800 mg, 99.97%) as a Brown yellow solid.

LC-MS: (M+H)⁺ found: 360.25.

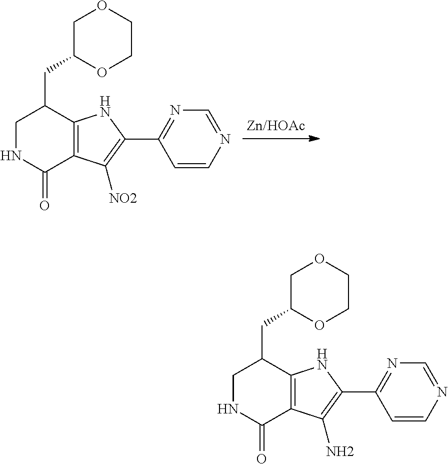 [SMILES: O=C1NCC(C[C@@H]2COCCO2)c2[nH]c(-c3ccncn3)c([N+](=O)[O-])c21 ; Nc1c(-c2ccncn2)[nH]c2c1C(=O)NCC2C[C@@H]1COCCO1]

A solution of 7-[(2R)-1,4-dioxan-2-ylmethyl]-3-nitro-2-(pyrimidin-4-yl)-1H,5H,6H,7H-pyrrolo[3,2-c]pyridin-4-one (800 mg, 2.226 mmol, 1 equiv) in HOAc (10 mL) was treated with Zn (728.10 mg, 1.130 mmol, 5 equiv) for 1 h at room temperature under nitrogen atmosphere. The resulting mixture was diluted with water (30 mL). The mixture was basified to pH 8 with NaOH (aq.). The resulting mixture was extracted with CH₂Cl₂ (5×300 mL). The combined organic layers were dried over anhydrous Na2SO4. After filtration, the filtrate was concentrated under reduced pressure. This resulted in 3-amino-7-[(2R)-1,4-dioxan-2-ylmethyl]-2-(pyrimidin-4-yl)-1H,5H,6H,7H-pyrrolo[3,2-c]pyridin-4-one (500 mg, 68.19%) as a yellow solid.

LC-MS: (M+H)⁺ found: 330.30.

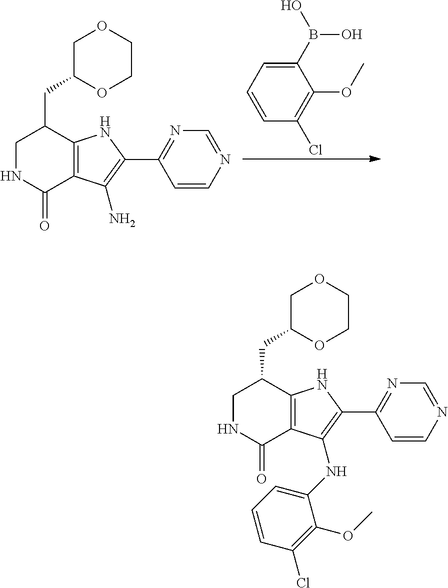 [SMILES: Nc1c(-c2ccncn2)[nH]c2c1C(=O)NCC2C[C@@H]1COCCO1 ; COc1c(Cl)cccc1B(O)O ; COc1c(Cl)cccc1Nc1c(-c2ccncn2)[nH]c2c1C(=O)NC[C@@H]2C[C@@H]1COCCO1]

A solution of 3-amino-7-[(2R)-1,4-dioxan-2-ylmethyl]-2-(pyrimidin-4-yl)-1H,5H,6H,7H-pyrrolo[3,2-c]pyridin-4-one (500 mg, 1.518 mmol, 1 equiv) and 3-chloro-2-methoxyphenylboronic acid (565.95 mg, 3.036 mmol, 2 equiv) in DCM (10 mL) was treated with Pyridine (240.16 mg, 3.036 mmol, 2 equiv) and TEA (307.23 mg, 3.036 mmol, 2 equiv) for 2 min at 0° C. under nitrogen atmosphere followed by the addition of Cu(OAc)2 (275.74 mg, 1.518 mmol, 1 equiv) in portions at room temperature. The resulting mixture was stirred for 2 h at room temperature under nitrogen atmosphere. The resulting mixture was extracted with CH₂Cl₂ (3×50 mL). The combined organic layers were washed with brine (2×50 mL), dried over anhydrous Na2SO4. After filtration, the filtrate was concentrated under reduced pressure. The residue was purified by reverse flash chromatography with the following conditions: Column: XBridge Prep OBD C18 Column, 30*150 mm, 5 μm; Mobile Phase A: Water (0.1% FA), Mobile Phase B: ACN; Flow rate: 60 mL/min; Gradient: 20% B to 50% B in 7 min; Wave Length: 254 nm; RT1 (min): 6.5. This resulted in (7S)-3-[(3-chloro-2-methoxyphenyl)amino]-7-[(2R)-1,4-dioxan-2-ylmethyl]-2-(pyrimidin-4-yl)-1H,5H,6H,7H-pyrrolo[3,2-c]pyridin-4-one (7.6 mg) as a yellow solid.

LC-MS: (M+H)⁺ found: 470.35.

¹H NMR (400 MHz, DMSO-d6) δ 12.00 (s, 1H), 9.05 (d, J=1.4 Hz, 1H), 8.56 (d, J=5.6 Hz, 1H), 7.96 (s, 1H), 7.20 (m, 2H), 6.86-6.76 (m, 2H), 6.31 (dd, J=6.8, 2.8 Hz, 1H), 3.93 (s, 3H), 3.83-3.39 (m, 8H), 3.19 (dd, J=11.3, 9.7 Hz, 1H), 3.09 (dd, J=8.8, 4.6 Hz, 1H), 1.78 (dt, J=14.1, 4.7 Hz, 1H), 1.67-1.55 (m, 1H).

### Example 105. (7S)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (compound 526)

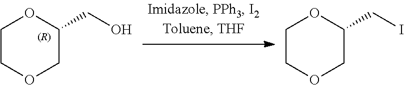

To a suspension of (R)-(1,4-dioxan-2-yl)methanol (100.00 g, 0.847 mol, 1.00 equiv) in Tolune (1000 mL) and THF (500 mL) was added imidazole (115.2 g, 1.69 mol, 2.00 equiv), PPh₃ (214.4 g, 0.847 mol, 1.00 equiv) at room temperature, I2 (214.4 g, 0.847 mol, 1.00 equiv) was added at 0 degrees. The mixture was stirred for 3 h at room temperature under nitrogen atmosphere. The reaction was monitored by GCMS, Desired product could be detected by GCMS. The resulting solution was quenching with 500 ml of Na2S2O3 saturated solution. The aqueous layer was extracted with EtOAc (3×250 mL), dried (Na2SO4) and concentrated. The resulting residue was purified on silica (EtOAc/PE=1:6) to provide 130 g (67%) of the title compound as a white oil.

LC-MS: (M+H)⁺ found: 229.

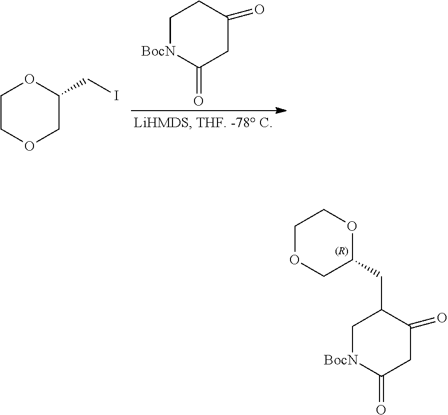

To a suspension of (S)-2-(iodomethyl)-1,4-dioxane (104.00 g, 0.457 mol, 1.40 equiv) and tert-butyl 2,4-dioxopiperidine-1-carboxylate (70.00 g, 0.327 mol, 1.00 equiv) in THF (1000 mL) was added LiHMDS (1.37 L, 1.37 mol, 3.00 equiv) at −60 degrees dropwise. The mixture was stirred for 1 h at 0 degrees under nitrogen atmosphere. The reaction was monitored by LCMS, Desired product could be detected by LCMS. The resulting solution was quenching with 500 ml of HCl (5%) at 0 degrees. The aqueous layer was extracted with EtOAc (3×250 mL), dried (Na2SO4) and concentrated. The resulting residue was purified on silica (0-80% PE/EtOAc) to provide 45 g (46%) of the title compound as a little brown oil.

LC-MS: (M+H)⁺ found: 314.

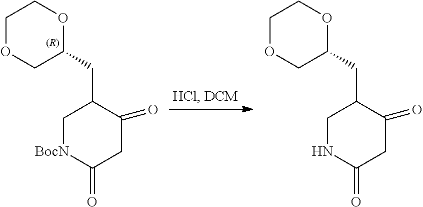

To a stirred mixture of tert-butyl 5-(((R)-1,4-dioxan-2-yl)methyl)-2,4-dioxopiperidine-1-carboxylate (45.00 g) in DCM (300 mL) was added HCl (150 mL) dropwise at 0 degrees C. under nitrogen atmosphere. The mixture was stirred at 0 degrees C. for 1 h. The resulting mixture was concentrated under reduced pressure to afford 30 g crude product, which was used to the next step without further purification.

LC-MS: (M+H)⁺ found: 214.

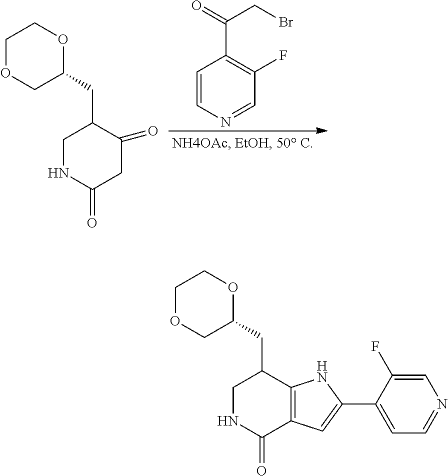 [SMILES: O=C1CC(=O)C(C[C@@H]2COCCO2)CN1 ; O=C(CBr)c1ccncc1F ; O=C1NCC(C[C@@H]2COCCO2)c2[nH]c(-c3ccncc3F)cc21]

To a stirred mixture of 5-[(2R)-1,4-dioxan-2-ylmethyl]piperidine-2,4-dione (1.5 g, 7.035 mmol, 1.00 equiv) and 2-bromo-1-(3-fluoropyridin-4-yl)ethanone (1.69 g, 7.739 mmol, 1.1 equiv) in EtOH (40 mL) were added NH₄OAc (5.42 g, 70.350 mmol, 10.00 equiv) in portions at room temperature under nitrogen atmosphere. The resulting mixture was stirred for 1 h at 50 degrees C. under nitrogen atmosphere. The resulting mixture was concentrated under reduced pressure. The aqueous layer was extracted with EtOAc (5×30 mL). The residue was purified by silica gel column chromatography, eluted with CH2Cl2/MeOH (20:1) to afford 7-[(2R)-1,4-dioxan-2-ylmethyl]-2-(3-fluoropyridin-4-yl)-1H,5H,6H,7H-pyrrolo[3,2-c]pyridin-4-one (1.02 g, 43.76%) as a white solid.

LCMS: [M+H]⁺ found: 332.

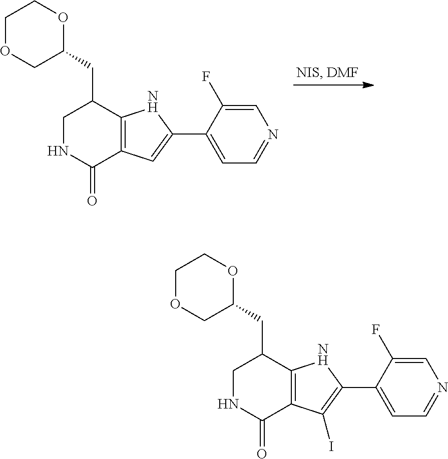 [SMILES: O=C1NCC(C[C@@H]2COCCO2)c2[nH]c(-c3ccncc3F)cc21 ; O=C1NCC(C[C@@H]2COCCO2)c2[nH]c(-c3ccncc3F)c(I)c21]

To a stirred mixture of 7-(1,4-dioxan-2-ylmethyl)-2-(3-fluoropyridin-4-yl)-1H,5H,6H,7H-pyrrolo[3,2-c]pyridin-4-one (970 mg, 2.927 mmol, 1.00 equiv) and in DMF (20 mL) were added NIS (790.36 mg, 3.512 mmol, 1.2 equiv) in portions at room temperature under nitrogen atmosphere. The resulting mixture was stirred for 4 h at room temperature under nitrogen atmosphere. The aqueous layer was extracted with EtOAc (5×30 mL). This resulted in 7-(1,4-dioxan-2-ylmethyl)-2-(3-fluoropyridin-4-yl)-3-iodo-1H,5H,6H,7H-pyrrolo[3,2-c]pyridin-4-one (1 g, 74.71%) as a yellow oil. The crude product was used in the next step directly without further purification.

LCMS: [M+H]⁺ found: 458.

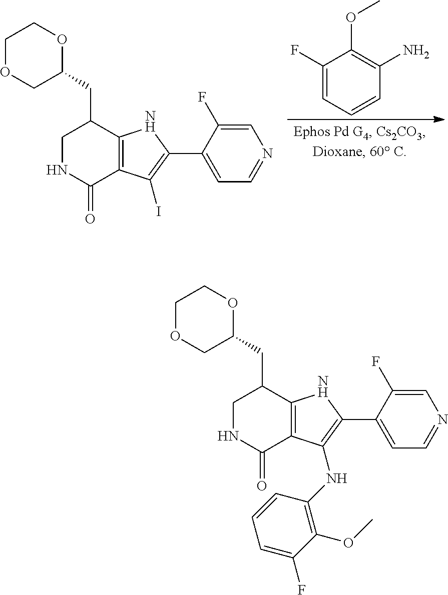

To a stirred mixture of 7-(1,4-dioxan-2-ylmethyl)-2-(3-fluoropyridin-4-yl)-3-iodo-1H,5H,6H,7H-pyrrolo[3,2-c]pyridin-4-one (1.17 g, 2.559 mmol, 1.00 equiv) and 3-fluoro-2-methoxyaniline (1.08 g, 7.677 mmol, 3 equiv) in DMF (15 mL) were added Cs2CO3 (1.67 g, 5.118 mmol, 2 equiv) and Ephos Pd G4 (0.47 g, 0.512 mmol, 0.2 equiv) in portions at room temperature under nitrogen atmosphere. The resulting mixture was stirred for 1 h at 60 degrees C. under nitrogen atmosphere. The aqueous layer was extracted with EtOAc (5×50 mL). The residue was purified by silica gel column chromatography, eluted with PE/EA (0:1) to afford 7-(1,4-dioxan-2-ylmethyl)-3-[(3-fluoro-2-methoxyphenyl)amino]-2-(3-fluoropyridin-4-yl)-1H,5H,6H,7H-pyrrolo[3,2-c]pyridin-4-one (1.07 g, 88.88%) as a yellow solid. This resulted in 7-(1,4-dioxan-2-ylmethyl)-3-[(3-fluoro-2-methoxyphenyl)amino]-2-(3-fluoropyridin-4-yl)-1H,5H,6H,7H-pyrrolo[3,2-c]pyridin-4-one (1.07 g, 88.88%) as a yellow solid.

LCMS: [M+H]⁺ found: 471.

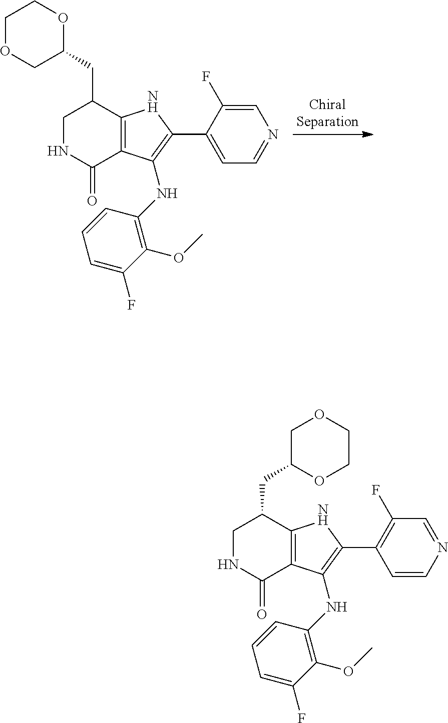 [SMILES: COc1c(F)cccc1Nc1c(-c2ccncc2F)[nH]c2c1C(=O)NCC2C[C@@H]1COCCO1 ; COc1c(F)cccc1Nc1c(-c2ccncc2F)[nH]c2c1C(=O)NC[C@@H]2C[C@@H]1COCCO1]

The crude product (100 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 30% B to 60% B in 7 min; Wave Length: 254 nm; RT1 (min): 6.5) to afford (7S)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (137.6 mg, NaN) as a white solid.

LCMS: [M+H]⁺ found: 471.

¹H NMR (300 MHz, Chloroform-d) δ 10.41 (s, 1H), 8.46 (d, J=4.9 Hz, 1H), 8.12 (d, J=5.8 Hz, 1H), 7.87 (s, 1H), 7.47-7.35 (m, 1H), 6.76-6.49 (m, 2H), 6.06 (d, J=7.8 Hz, 1H), 5.29 (s, 1H), 4.12-3.97 (m, 3H), 3.95-3.44 (m, 8H), 3.35 (d, J=8.4 Hz, 2H), 2.13-2.01 (m, 1H), 1.74-1.66 (m, 1H).

### Example 106. (7S)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (compound 552)

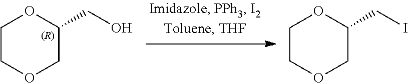

To a suspension of (R)-(1,4-dioxan-2-yl)methanol (100.00 g, 0.847 mol, 1.00 equiv) in Tolune (1000 mL) and THF (500 mL) was added imidazole (115.2 g, 1.69 mol, 2.00 equiv), PPh₃ (214.4 g, 0.847 mol, 1.00 equiv) at room temperature, I₂ (214.4 g, 0.847 mol, 1.00 equiv) was added at 0 degrees C. The mixture was stirred for 3 h at room temperature under nitrogen atmosphere.

Desired product could be detected by LCMS. The resulting solution was quenching with 500 ml of Na₂S₂O₃ saturated solution. The aqueous layer was extracted with EtOAc (3×250 mL), dried (Na₂SO₄) and concentrated. The resulting residue was purified on silica (EtOAc/PE=1:6) to provide 130 g (67%) of the title compound as a colorless oil.

LC-MS: (M+H)⁺ 229.

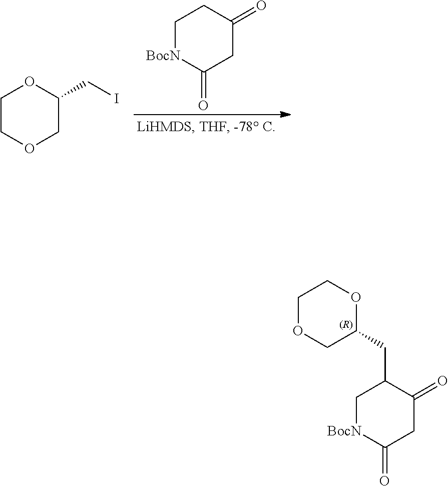

To a suspension of (S)-2-(iodomethyl)-1,4-dioxane (104.00 g, 0.457 mol, 1.40 equiv) and tert-butyl 2,4-dioxopiperidine-1-carboxylate (70.00 g, 0.327 mol, 1.00 equiv) in THF (1000 mL) was dropwise added LiHMDS (1.37 L, 1.37 mol, 3.00 equiv) at −60° C. The mixture was stirred for 1 h at 0 degrees under nitrogen atmosphere.

Desired product could be detected by LCMS. The resulting solution was quenching with 500 ml of HCl (5%) at 0 degrees. The aqueous layer was extracted with EtOAc (3×250 mL), dried (Na₂SO₄) and concentrated. The resulting residue was purified by silica gel column (PE/EtOAc, 1:1) to provide 45 g (46%) of the title compound as a light brown oil.

LC-MS: (M+H)⁺ found: 314.

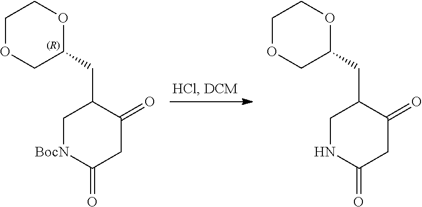

To a stirred mixture of tert-butyl 5-(((R)-1,4-dioxan-2-yl)methyl)-2,4-dioxopiperidine-1-carboxylate (45.00 g) in DCM (300 mL) was added HCl (150 mL) dropwise at 0 degrees C. under nitrogen atmosphere. The mixture was stirred at 0 degrees C. for 1 h. The resulting mixture was concentrated under reduced pressure to afford 30 g crude product, which was used to the next step without further purification.

LC-MS: (M+H)⁺ found: 214.

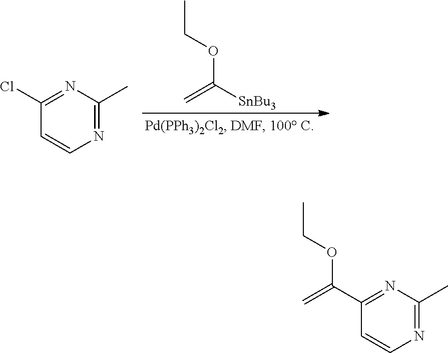

A mixture of 4-chloro-2-methylpyrimidine (20.00 g, 156.2 mmol, 1.00 equiv), tributyl(1-ethoxyvinyl)stannane (45.82 g, 468 mmol, 3.00 equiv) and Pd(PPh₃)₂Cl₂ (8.86 g, 15.6 mmol, 0.10 equiv) in DMF (200.00 mL) was stirred for 12 h at 100 degrees C. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EtOAc (5:1) to afford 4-(1-ethoxyvinyl)-2-methylpyrimidine 17 g (66%) as a yellow oil.

LC-MS: M+H found: 165.0.

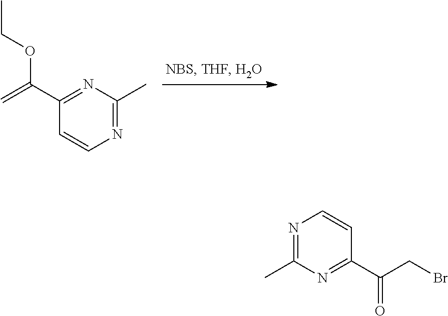

A mixture of 4-(1-ethoxyvinyl)-2-methylpyrimidine (17.00 g, 103.0 mmol, 1.00 equiv), NBS (30.13 g, 133.9 mmol, 1.30 equiv) and H₂O (15 mL) in THF (150 mL) was stirred for 1 h at room temperature. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. This resulted in 2-bromo-1-(2-methylpyrimidin-4-yl) ethan-1-one 18 g crude as a brown oil.

LC-MS: (M+H)⁺ found 215.

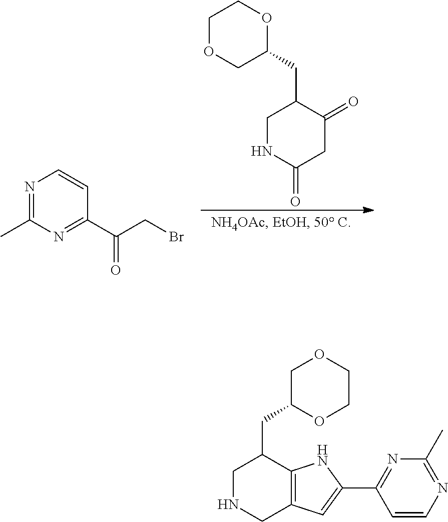 [SMILES: Cc1nccc(C(=O)CBr)n1 ; O=C1CC(=O)C(C[C@@H]2COCCO2)CN1 ; Cc1nccc(-c2cc3c([nH]2)C(C[C@@H]2COCCO2)CNC3)n1]

A mixture of 5-(((R)-1,4-dioxan-2-yl)methyl)piperidine-2,4-dione (14 g, 83 mmol, 1.0 equiv), 2-bromo-1-(2-methylpyrimidin-4-yl)ethan-1-one (18 g, 107 mmol, 1.3 equiv), and NH₄OAc (50.3 g, 830 mmol, 10 equiv) in EtOH (150.0 mL) was stirred at 50 degrees C. for 3 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (10:1)) to afford 7-(((R)-1,4-dioxan-2-yl)methyl)-2-(2-methylpyrimidin-4-yl)-4,5,6,7-tetrahydro-1H-pyrrolo[3,2-c]pyridine (9 g, 57%) as a yellow solid.

LC-MS: (M+H)⁺ found: 329.

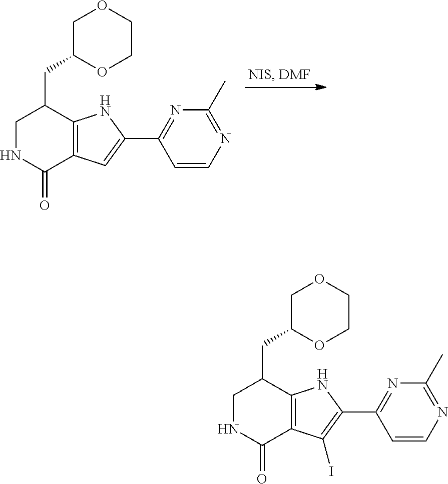 [SMILES: Cc1nccc(-c2cc3c([nH]2)C(C[C@@H]2COCCO2)CNC3=O)n1 ; Cc1nccc(-c2[nH]c3c(c2I)C(=O)NCC3C[C@@H]2COCCO2)n1]

To a stirred mixture of 7-(((R)-1,4-dioxan-2-yl)methyl)-2-(2-methylpyrimidin-4-yl)-1,5,6,7-tetrahydro-4H-pyrrolo[3,2-c]pyridin-4-one (17 g, 51.6 mmol, 1.00 equiv) in DMF (150 mL) was added NIS (15.1 g, 67.8 mmol, 1.3 equiv) at 0 degrees C. The mixture was stirred at room temperature for 2 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (20:1)) to afford 7-(((R)-1,4-dioxan-2-yl)methyl)-3-iodo-2-(2-methylpyrimidin-4-yl)-1,5,6,7-tetrahydro-4H-pyrolo[3,2-c]pyridin-4-one (16 g, 53%) as yellow solid.

LC-MS: (M+H)⁺ found: 455.

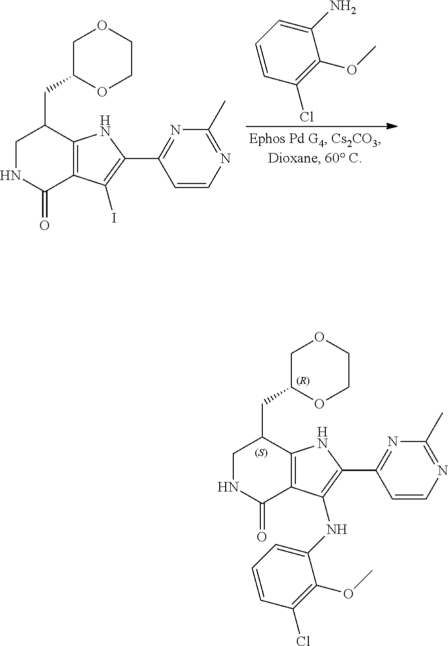

A mixture of 7-[(2R)-1,4-dioxan-2-ylmethyl]-3-iodo-2-(2-methylpyrimidin-4-yl)-1H,5H,6H,7H-pyrrolo[3,2-c]pyridin-4-one (35 g, 77.047 mmol, 1 equiv), Cs₂CO₃ (50.21 g, 154 mmol, 2 equiv), 3-chloro-2-methoxyaniline (14.57 g, 92.456 mmol, 1.2 equiv) and EPhos Pd G4 (10.62 g, 11.557 mmol, 0.15 equiv) in DMF was stirred for 3 h at 60° C. under nitrogen atmosphere. The reaction was quenched with Water at room temperature. The resulting mixture was extracted with EtOAc (2×200 mL). The combined organic layers were washed with water (2×200 mL), dried over anhydrous Na2SO4. After filtration, the filtrate was concentrated under reduced pressure. The crude product (5 g) was purified by Prep-SFC with the following conditions (Column: CHIRALPAK IB N-3, 4.6*100 mm, 3 μm; Mobile Phase B: MeOH (0.1% DEA; Flow rate: 2 mL/min; Gradient: isocratic 10% B; Wave Length: 220 nm) to afford (7S)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (3.3716 g, 9.04%) as a yellow solid.

LC-MS: (M+H)⁺ found: 484.

¹H NMR (400 MHz, Chloroform-d) δ 11.30-11.26 (m, 1H), 8.40-8.29 (m, 1H), 760-7.55 (m, 1H), 6.85-6.68 (m, 3H), 6.50-6.29 (m, 1H), 5.70-5.52 (m, 1H), 4.25-4.14 (m, 1H), 4.13-4.05 (m, 3H), 4.05-3.73 (m, 5H), 3.41-3.35 (m, 4H), 2.81-2.66 (m, 3H), 1.91-1.77 (m, 1H), 1.66-1.52 (m, 1H).

### Example 107. (7R*)-3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (2.7 mg, 6.75%). This resulted in (7R*)-3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (compound 565)

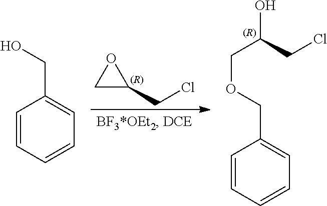

To a stirred solution/mixture of benzyl alcohol (1 g, 9.247 mmol, 1 equiv) and (−)-epichlorohydrin (0.94 g, 10.172 mmol, 1.1 equiv) in DCE (20 mL, 252.653 mmol, 27.32 equiv) was added BF3*Et2O (0.13 g, 0.925 mmol, 0.1 equiv) dropwise at 0° C. under nitrogen atmosphere. The mixture was stirred for overnight at room temperature under nitrogen atmosphere. The reaction was quenched by the addition of Water (20 mL) at 0° C. The resulting mixture was extracted with EtOAc (3×20 mL). The combined organic layers dried over anhydrous Na2SO4. After filtration, the filtrate was concentrated under reduced pressure. The crude product was purified by reverse phase flash with the following conditions (ACN/H2O=3/7) to afford (2R)-1-(benzyloxy)-3-chloropropan-2-ol (800 mg, 43.11%) as a white solid.

LC-MS: (M+H)⁺ found: 201.

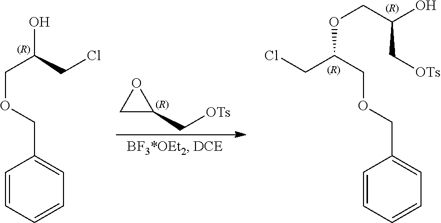

To a stirred solution of (2R)-1-(benzyloxy)-3-chloropropan-2-ol (72 g, 358.816 mmol, 1 equiv) and (2R)-oxiran-2-ylmethyl 4-methylbenzenesulfonate (24.57 g, 107.645 mmol, 0.3 equiv) in DCE (1.5 L) was added BF3*Et2O (4.55 mL, 35.882 mmol, 0.1 equiv) dropwise at 0° C. under nitrogen atmosphere. The resulting mixture was stirred for overnight at room temperature under nitrogen atmosphere. The reaction was monitored by LCMS. The residue was washed with saturated sodium bicarbonate solution (2×1 L). The combined organic layers were dried over anhydrous Na2SO4. After filtration, the filtrate was concentrated under reduced pressure. The residue was purified by reverse flash chromatography with the following conditions: column, silica gel; mobile phase, MeCN in water, 40% to 50% gradient in 10 min; detector, UV 254 nm. This resulted in (2R)-1-{[(2R)-1-(benzyloxy)-3-chloropropan-2-yl]oxy}-3-[(4-methylbenzenesulfonyl)oxy]propan-2-ol (34 g, 22.09%) as a colorless oil.

LC-MS: (M+H)⁺ found: 429.

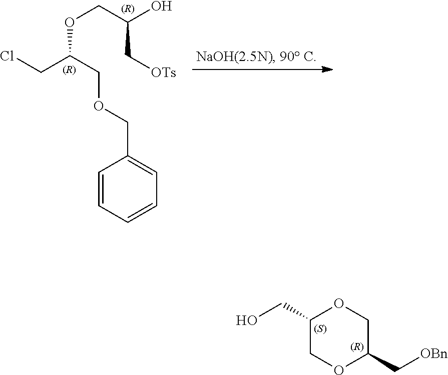

A solution of (2R)-1-{[(2R)-1-(benzyloxy)-3-chloropropan-2-yl]oxy}-3-[(4-methylbenzenesulfonyl)oxy]propan-2-ol (34 g, 79.269 mmol, 1 equiv) in NaOH (130 mL, 1.5N) was stirred for overnight at room temperature under nitrogen atmosphere. Then it was heated to 90° C. stirred for 4 h under nitrogen atmosphere. After that the reaction was cooled to room temperature and stirred for overnight. The mixture was then re-heated to 90° C. for about 2 h and finally cooled to room temperature. The mixture was acidified to pH 6 with 1N HCl (aq.). The resulting mixture was extracted with EtOAc (3×mL). The combined organic layers were washed with brine (2×2 mL), dried over anhydrous Na2SO4. After filtration, the filtrate was concentrated under reduced pressure. The residue was purified by reverse flash chromatography with the following conditions: column, silica gel; mobile phase, MeCN in water, 50% to 60% gradient in 10 min; detector, UV 254 nm. This resulted in [(2S,5R)-5-[(benzyloxy)methyl]-1,4-dioxan-2-yl]methanol (6.1 g, 32.29%) as a colorless oil.

LC-MS: (M+H)⁺ found: 239.

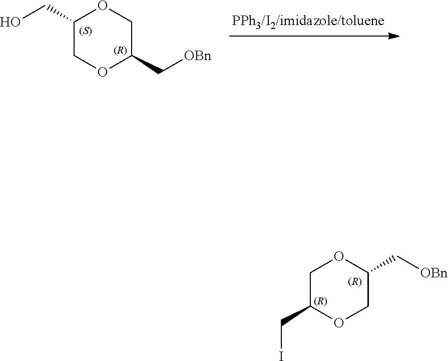

To a stirred solution of [(2S,5R)-5-[(benzyloxy)methyl]-1,4-dioxan-2-yl]methanol (1 g, 4.197 mmol, 1 equiv) and Imidazole (0.57 g, 8.394 mmol, 2 equiv) in Toluene (8 mL) were added PPh₃ (1.10 g, 4.197 mmol, 1 equiv) and iodine (1.07 g, 4.197 mmol, 1 equiv) in portions at room temperature under nitrogen atmosphere. The resulting mixture was stirred for 1 h at room temperature under nitrogen atmosphere. To the above mixture was added THF (4 mL) dropwise at room temperature. The resulting mixture was stirred for additional 1 h at room temperature. The reaction was monitored by LCMS. The reaction was quenched by the addition of sat. sodium hyposulfite (aq.) (5 mL) at room temperature. The resulting mixture was extracted with EtOAc (3×20 mL). The combined organic layers were washed with brine (2×2 mL), dried over anhydrous Na2SO4. After filtration, the filtrate was concentrated under reduced pressure. The residue was purified by silica gel column chromatography, eluted with PE/EA (10:1) to afford (2R,5R)-2-[(benzyloxy)methyl]-5-(iodomethyl)-1,4-dioxane (1.3 g, 88.97%) as a colorless oil.

LC-MS: (M+H)⁺ found: 349.

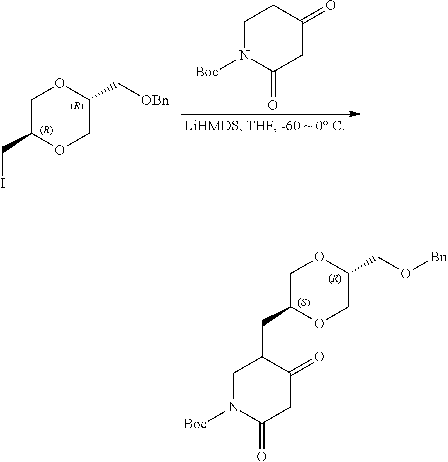

To a stirred mixture of (2R,5R)-2-[(benzyloxy)methyl]-5-(iodomethyl)-1,4-dioxane (1 g, 2.872 mmol, 1 equiv) and tert-butyl 2,4-dioxopiperidine-1-carboxylate (0.73 g, 3.446 mmol, 1.2 equiv) in THF (20 mL) was added LiHMDS (1.44 g, 8.616 mmol, 3 equiv) dropwise at −20° C. under nitrogen atmosphere. The resulting mixture was stirred for 2 h at −20° C. Desired product could be detected by LCMS. The mixture was acidified to pH 5 with HCl (aq.). The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (4:1) to afford tert-butyl 5-{[(2S,5R)-5-[(benzyloxy)methyl]-1,4-dioxan-2-yl]methyl}-2,4-dioxopiperidine-1-carboxylate (350 mg, 28.11%) as a light green oil.

LC-MS: (M+H)⁺ found: 434.

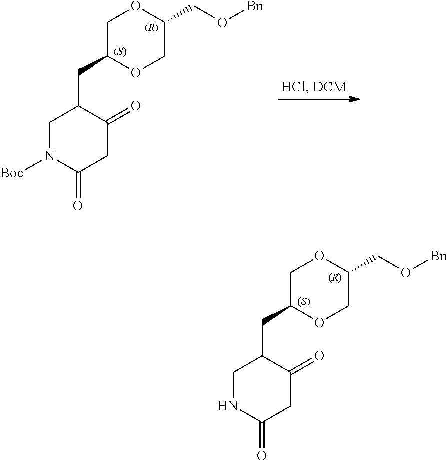

Into a 100 mL round-bottom flask were added tert-butyl 5-{[(2S,5R)-5-[(benzyloxy)methyl]-1,4-dioxan-2-yl]methyl}-2,4-dioxopiperidine-1-carboxylate (1 g, 2.307 mmol, 1 equiv), HCl (gas) in 1,4-dioxane (10 mL) and DCM (20 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:2) to afford 5-{[(2S,5R)-5-[(benzyloxy)methyl]-1,4-dioxan-2-yl]methyl}piperidine-2,4-dione (650 mg, 84.52%) as a yellow solid.

LC-MS: (M+H)⁺ found: 334.

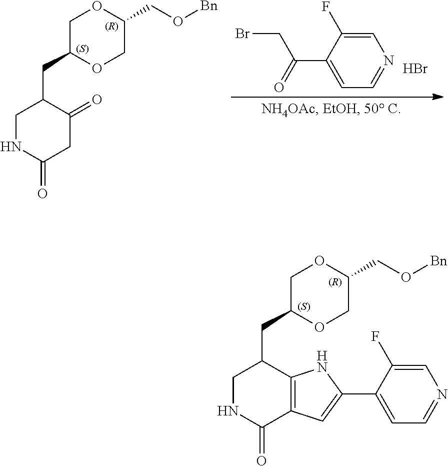

Into a 100 mL round-bottom flask were added 5-{[(2S,5R)-5-[(benzyloxy)methyl]-1,4-dioxan-2-yl]methyl}piperidine-2,4-dione (500 mg, 1.500 mmol, 1 equiv), 2-bromo-1-(3-fluoropyridin-4-yl)ethanone (490.48 mg, 2.250 mmol, 1.5 equiv), NH₄OAc (1156.07 mg, 15.000 mmol, 10 equiv) and EtOH (10 mL) at room temperature. The mixture was stirred for 5 h at 50 degrees C. under nitrogen atmosphere. After the reaction, The resulting mixture was concentrated under reduced pressure. The residue was purified by silica gel column chromatography, eluted with PE/EtOAc (1:1) to afford 7-{[(2S,5R)-5-[(benzyloxy)methyl]-1,4-dioxan-2-yl]methyl}-2-(3-fluoropyridin-4-yl)-1H,5H,6H,7H-pyrrolo[3,2-c]pyridin-4-one (400 mg, 59.07%) as a yellow solid.

LC-MS: (M+H)⁺ found: 452.

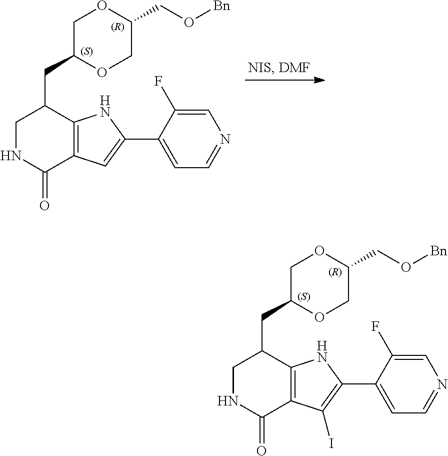

To a stirred solution of 7-{[(2S,5R)-5-[(benzyloxy)methyl]-1,4-dioxan-2-yl]methyl}-2-(3-fluoropyridin-4-yl)-1H,5H,6H,7H-pyrrolo[3,2-c]pyridin-4-one (500 mg, 1.107 mmol, 1 equiv) in DMF (10 mL) was added NIS (298.98 mg, 1.328 mmol, 1.2 equiv) in portions at −35° C. under nitrogen atmosphere. The reaction solution was stirred at room temperature for 1 h, The resulting mixture was extracted with EtOAc (4×40 mL). The combined organic layers were washed with brine (1×20 mL), dried over anhydrous Na2SO4. After filtration, the filtrate was concentrated under reduced pressure. The residue was purified by Prep-TLC (PE/EtOAc 1:1) to afford 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (420 mg, 65.68%) as a white solid.

LC-MS: (M+H)⁺ found: 578.

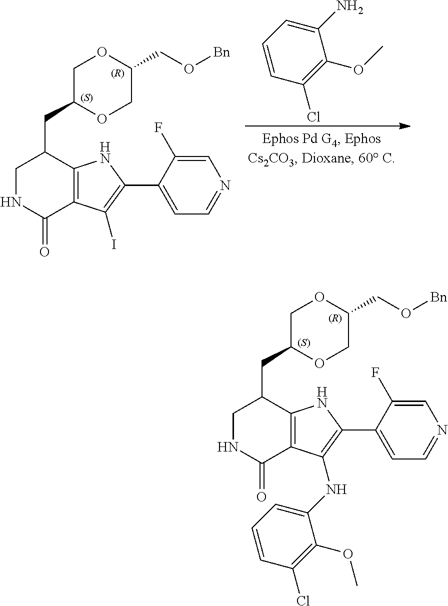

To a solution of 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (400 mg, 0.693 mmol, 1 equiv) and Cs₂CO₃ (451.43 mg, 1.386 mmol, 2 equiv) in DMF (10 mL) were added EPhos Pd G4 (63.64 mg, 0.069 mmol, 0.1 equiv) and 3-chloro-2-methoxyaniline (131.02 mg, 0.832 mmol, 1.2 equiv) at room temperature under argon atmosphere. The resulting mixture was stirred for 4 h at 50 degrees C. under argon atmosphere. The resulting mixture was concentrated under reduced pressure. The residue was purified by silica gel column chromatography, eluted with CH2Cl2/MeOH (30:1) to afford 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (320 mg, 76.09%) as a yellow solid.

LC-MS: (M+H)⁺ found: 607.

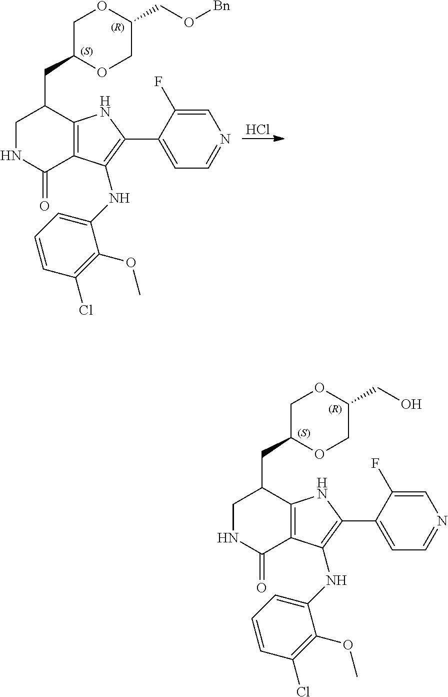

Into a 100 mL round-bottom flask were added 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (100 mg, 0.165 mmol, 1 equiv) and HCl (gas) in 1,4-dioxane (10 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:4) to afford 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (80 mg, 93.95%) as a yellow solid.

LC-MS: (M+H)⁺ found: 517.

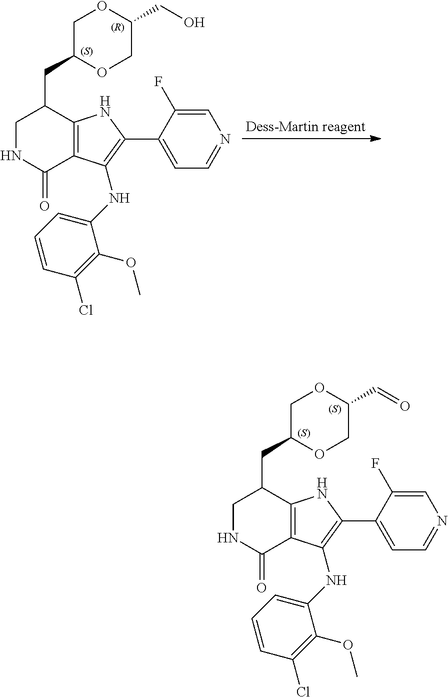

Into a 50 mL round-bottom flask were added 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (100 mg, 0.193 mmol, 1 equiv), Dess-Martin (90.25 mg, 0.212 mmol, 1.1 equiv) and DCM (10 mL) at room temperature. The mixture was stirred 1 h at room temperature under nitrogen atmosphere. After the reaction, the mixture was concentrated under reduced pressure to afford (2S,5S)-5-({3-[(3-chloro-2-methoxyphenyl)amino]-2-(3-fluoropyridin-4-yl)-4-oxo-1H,5H,6H,7H-pyrrolo[3,2-c]pyridin-7-yl}methyl)-1,4-dioxane-2-carbaldehyde (95 mg, 95.37%) as a yellow solid.

LC-MS: (M+H)⁺ found 515.

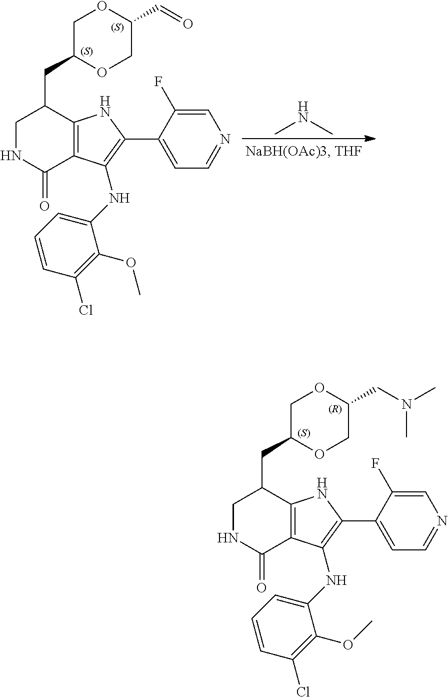

To a stirred mixture of (2S,5S)-5-((3-[(3-chloro-2-methoxyphenyl)amino]-2-(3-fluoropyridin-4-yl)-4-oxo-1H,5H,6H,7H-pyrrolo[3,2-c]pyridin-7-yl)methyl)-1,4-dioxane-2-carbaldehyde (50 mg, 0.097 mmol, 1 equiv) and dimethylamine (5.25 mg, 0.116 mmol, 1.2 equiv) in THF (5 mL) was added NaBH(OAc)3 (20.58 mg, 0.097 mmol, 1 equiv) dropwise at −20° C. under nitrogen atmosphere. The resulting mixture was stirred for 2 h at −20° C. Desired product could be detected by LCMS. The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (2:3) to afford 3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (30 mg, 56.79%) as a light yellow oil.

LC-MS: (M+H)⁺ found: 544.

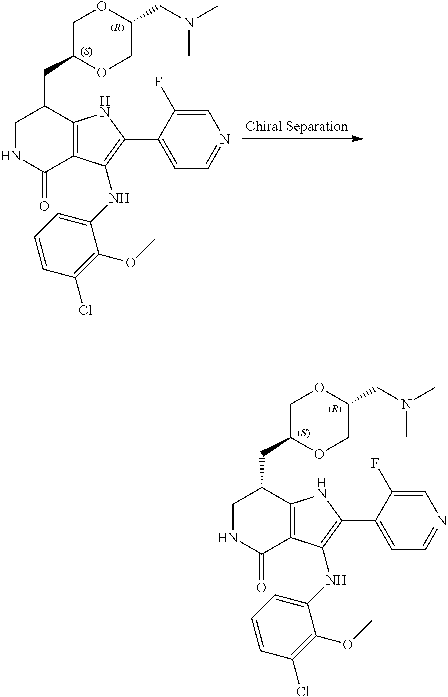

3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (40 mg, 0.074 mmol, 1 equiv) was used for chiral separation (Column: CHIRALPAK IG, 2*25 cm, 5 μm; Mobile Phase A: Hex:DCM=3:1 (0.5% 2M NH3-MeOH)-HPLC, Mobile Phase B: IPA-HPLC; Flow rate: 20 mL/min; Gradient: 20% B to 20% B in 19 min; Wave Length: 220/254 nm; RT1 (min): 7.92; RT2 (min): 9.81; Sample Solvent: ETOH:DCM=1:1; Injection Volume: 0.8 mL; Number Of Runs: 4), and 2 peak was splitted from the material, the prepeak is the product (7R*)-3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (2.7 mg, 6.75%). This resulted in (7R*)-3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (2.7 mg, 6.75%) as a yellow solid.

LC-MS: (M+H)⁺ found: 544.

¹H NMR (400 MHz, Methanol-d4) δ 8.43 (d, J=3.5 Hz, 1H), 8.18 (d, J=5.3 Hz, 1H), 7.52 (dd, J=6.9, 5.3 Hz, 1H), 6.74-6.61 (m, 2H), 6.23 (dd, J=8.0, 1.6 Hz, 1H), 3.98 (s, 3H), 3.92 (dd, J=11.6, 2.6 Hz, 1H), 3.83-3.72 (m, 3H), 3.71 (d, J=5.1 Hz, 1H), 3.68-3.56 (m, 1H), 3.49-3.37 (m, 2H), 3.26 (td, J=7.2, 3.5 Hz, 1H), 2.59-2.50 (m, 1H), 2.50-2.43 (m, 1H), 2.41 (s, 6H), 1.87 (ddd, J=14.3, 7.6, 3.5 Hz, 1H), 1.75 (ddd, J=14.6, 8.5, 6.7 Hz, 1H).

### Example 108. (7R*)-3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (5 mg, 12.50%). This resulted in (7R*)-3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (compound 564)

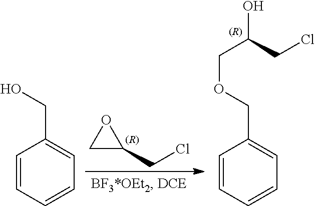

To a stirred solution/mixture of benzyl alcohol (1 g, 9.247 mmol, 1 equiv) and (−)-epichlorohydrin (0.94 g, 10.172 mmol, 1.1 equiv) in DCE (20 mL, 252.653 mmol, 27.32 equiv) was added BF3*Et2O (0.13 g, 0.925 mmol, 0.1 equiv) dropwise at 0° C. under nitrogen atmosphere. The mixture was stirred for overnight at room temperature under nitrogen atmosphere. The reaction was quenched by the addition of Water (20 mL) at 0° C. The resulting mixture was extracted with EtOAc (3×20 mL). The combined organic layers dried over anhydrous Na2SO4. After filtration, the filtrate was concentrated under reduced pressure. The crude product was purified by reverse phase flash with the following conditions (ACN/H2O=3/7) to afford (2R)-1-(benzyloxy)-3-chloropropan-2-ol (800 mg, 43.11%) as a white solid.

LC-MS: (M+H)⁺ found: 201.

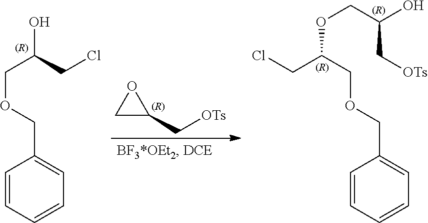

To a stirred solution of (2R)-1-(benzyloxy)-3-chloropropan-2-ol (72 g, 358.816 mmol, 1 equiv) and (2R)-oxiran-2-ylmethyl 4-methylbenzenesulfonate (24.57 g, 107.645 mmol, 0.3 equiv) in DCE (1.5 L) was added BF3*Et2O (4.55 mL, 35.882 mmol, 0.1 equiv) dropwise at 0° C. under nitrogen atmosphere. The resulting mixture was stirred for overnight at room temperature under nitrogen atmosphere. The reaction was monitored by LCMS. The residue was washed with saturated sodium bicarbonate solution (2×1 L). The combined organic layers were dried over anhydrous Na2SO4. After filtration, the filtrate was concentrated under reduced pressure. The residue was purified by reverse flash chromatography with the following conditions: column, silica gel; mobile phase, MeCN in water, 40% to 50% gradient in 10 min; detector, UV 254 nm. This resulted in (2R)-1-{[(2R)-1-(benzyloxy)-3-chloropropan-2-yl]oxy}-3-[(4-methylbenzenesulfonyl)oxy]propan-2-ol (34 g, 22.09%) as a colorless oil.

LC-MS: (M+H)⁺ found: 429.

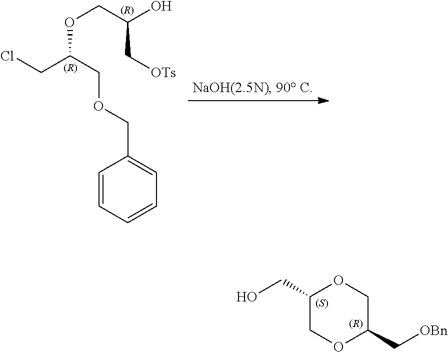

A solution of (2R)-1-{[(2R)-1-(benzyloxy)-3-chloropropan-2-yl]oxy}-3-[(4-methylbenzenesulfonyl)oxy]propan-2-ol (34 g, 79.269 mmol, 1 equiv) in NaOH (130 mL, 1.5N) was stirred for overnight at room temperature under nitrogen atmosphere. Then it was heated to 90° C. stirred for 4 h under nitrogen atmosphere. After that the reaction was cooled to room temperature and stirred for overnight. The mixture was then re-heated to 90° C. for about 2 h and finally cooled to room temperature. The mixture was acidified to pH 6 with 1N HCl (aq.). The resulting mixture was extracted with EtOAc (3×mL). The combined organic layers were washed with brine (2×2 mL), dried over anhydrous Na2SO4. After filtration, the filtrate was concentrated under reduced pressure. The residue was purified by reverse flash chromatography with the following conditions: column, silica gel; mobile phase, MeCN in water, 50% to 60% gradient in 10 min; detector, UV 254 nm. This resulted in [(2S,5R)-5-[(benzyloxy)methyl]-1,4-dioxan-2-yl]methanol (6.1 g, 32.29%) as a colorless oil.

LC-MS: (M+H)⁺ found: 239.

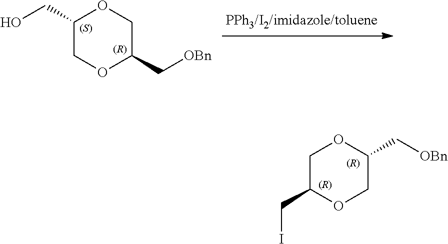

To a stirred solution of [(2S,5R)-5-[(benzyloxy)methyl]-1,4-dioxan-2-yl]methanol (1 g, 4.197 mmol, 1 equiv) and Imidazole (0.57 g, 8.394 mmol, 2 equiv) in Toluene (8 mL) were added PPh₃ (1.10 g, 4.197 mmol, 1 equiv) and iodine (1.07 g, 4.197 mmol, 1 equiv) in portions at room temperature under nitrogen atmosphere. The resulting mixture was stirred for 1 h at room temperature under nitrogen atmosphere. To the above mixture was added THF (4 mL) dropwise at room temperature. The resulting mixture was stirred for additional 1 h at room temperature. The reaction was monitored by LCMS. The reaction was quenched by the addition of sat. sodium hyposulfite (aq.) (5 mL) at room temperature. The resulting mixture was extracted with EtOAc (3×20 mL). The combined organic layers were washed with brine (2×2 mL), dried over anhydrous Na2SO4. After filtration, the filtrate was concentrated under reduced pressure. The residue was purified by silica gel column chromatography, eluted with PE/EA (10:1) to afford (2R,5R)-2-[(benzyloxy)methyl]-5-(iodomethyl)-1,4-dioxane (1.3 g, 88.97%) as a colorless oil.

LC-MS: (M+H)⁺ found: 349.

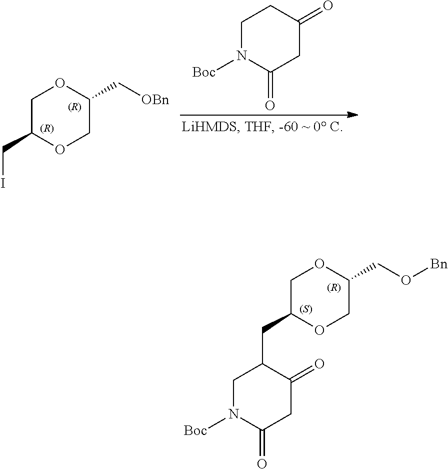

To a stirred mixture of (2R,5R)-2-[(benzyloxy)methyl]-5-(iodomethyl)-1,4-dioxane (1 g, 2.872 mmol, 1 equiv) and tert-butyl 2,4-dioxopiperidine-1-carboxylate (0.73 g, 3.446 mmol, 1.2 equiv) in THF (20 mL) was added LiHMDS (1.44 g, 8.616 mmol, 3 equiv) dropwise at −20° C. under nitrogen atmosphere. The resulting mixture was stirred for 2 h at −20° C. Desired product could be detected by LCMS. The mixture was acidified to pH 5 with HCl (aq.). The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (4:1) to afford tert-butyl 5-{[(2S,5R)-5-[(benzyloxy)methyl]-1,4-dioxan-2-yl]methyl}-2,4-dioxopiperidine-1-carboxylate (350 mg, 28.11%) as a light green oil.

LC-MS: (M+H)⁺ found: 434.

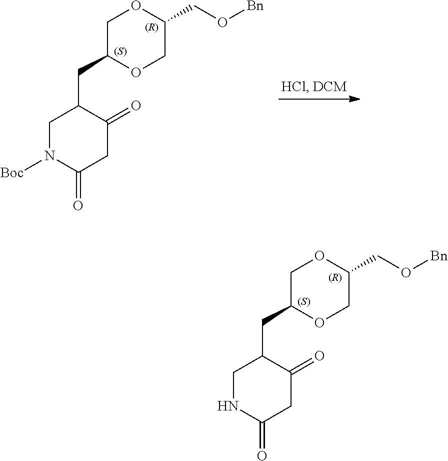

Into a 100 mL round-bottom flask were added tert-butyl 5-{[(2S,5R)-5-[(benzyloxy)methyl]-1,4-dioxan-2-yl]methyl}-2,4-dioxopiperidine-1-carboxylate (1 g, 2.307 mmol, 1 equiv), HCl (gas) in 1,4-dioxane (10 mL) and DCM (20 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:2) to afford 5-{[(2S,5R)-5-[(benzyloxy)methyl]-1,4-dioxan-2-yl]methyl}piperidine-2,4-dione (650 mg, 84.52%) as a yellow solid.

LC-MS: (M+H)⁺ found: 334.

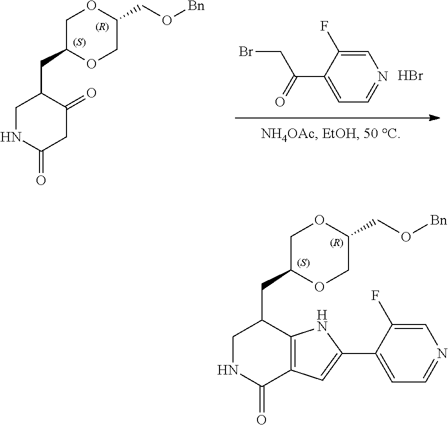

Into a 100 mL round-bottom flask were added 5-1[{2S,5R)-5-[(benzyloxy)methyl]-1,4-dioxan-2-yl]methyl}piperidine-2,4-dione (500 mg, 1.500 mmol, 1 equiv), 2-bromo-1-(3-fluoropyridin-4-yl)ethanone (490.48 mg, 2.250 mmol, 1.5 equiv), NH4OAc (1156.07 mg, 15.000 mmol, 10 equiv) and EtOH (10 mL) at room temperature. The mixture was stirred for 5 h at 50 degrees C. under nitrogen atmosphere. After the reaction, The resulting mixture was concentrated under reduced pressure. The residue was purified by silica gel column chromatography, eluted with PE/EtOAc (1:1) to afford 7-{[(2S,5R)-5-[(benzyloxy)methyl]-1,4-dioxan-2-yl]methyl}-2-(3-fluoropyridin-4-yl)-1H,5H,6H,7H-pyrrolo[3,2-c]pyridin-4-one (400 mg, 59.07%) as a yellow solid.

LC-MS: (M+H)⁺ found: 452.

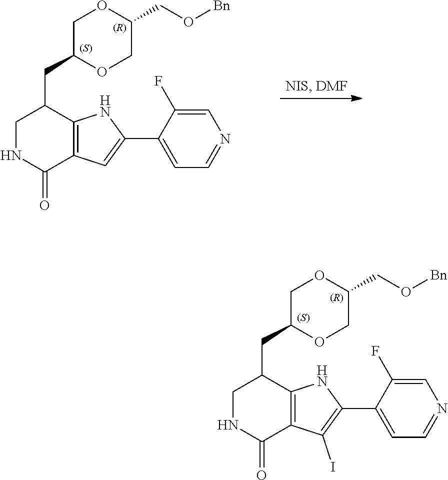

To a stirred solution of 7-{[(2S,5R)-5-[(benzyloxy)methyl]-1,4-dioxan-2-yl]methyl}-2-(3-fluoropyridin-4-yl)-1H,5H,6H,7H-pyrrolo[3,2-c]pyridin-4-one (500 mg, 1.107 mmol, 1 equiv) in DMF (10 mL) was added NIS (298.98 mg, 1.328 mmol, 1.2 equiv) in portions at −35° C. under nitrogen atmosphere. The reaction solution was stirred at room temperature for 1 h, The resulting mixture was extracted with EtOAc (4×40 mL). The combined organic layers were washed with brine (1×20 mL), dried over anhydrous Na2SO4. After filtration, the filtrate was concentrated under reduced pressure. The residue was purified by Prep-TLC (PE/EtOAc 1:1) to afford 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (420 mg, 65.68%) as a white solid.

LC-MS: (M+H)⁺ found: 578.

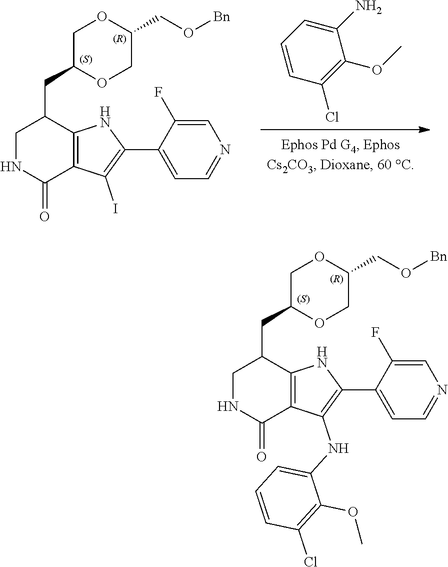

To a solution of 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (400 mg, 0.693 mmol, 1 equiv) and Cs2CO3 (451.43 mg, 1.386 mmol, 2 equiv) in DMF (10 mL) were added EPhos Pd G4 (63.64 mg, 0.069 mmol, 0.1 equiv) and 3-chloro-2-methoxyaniline (131.02 mg, 0.832 mmol, 1.2 equiv) at room temperature under argon atmosphere. The resulting mixture was stirred for 4 h at 50 degrees C. under argon atmosphere. The resulting mixture was concentrated under reduced pressure. The residue was purified by silica gel column chromatography, eluted with CH2Cl2/MeOH (30:1) to afford 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (320 mg, 76.09%) as a yellow solid.

LC-MS: (M+H)⁺ found: 607.

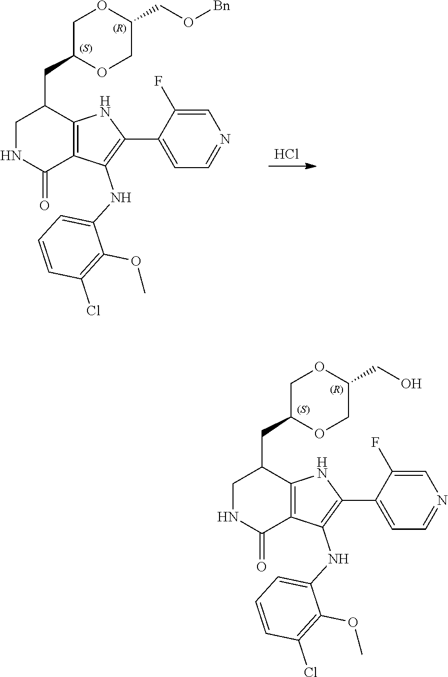

Into a 100 mL round-bottom flask were added 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (100 mg, 0.165 mmol, 1 equiv) and HCl (gas) in 1,4-dioxane (10 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:4) to afford 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (80 mg, 93.95%) as a yellow solid.

LC-MS: (M+H)⁺ found: 517.

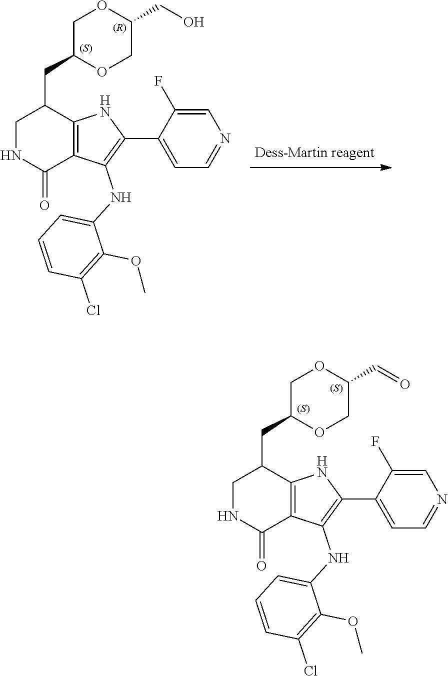

Into a 50 mL round-bottom flask were added 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (100 mg, 0.193 mmol, 1 equiv), Dess-Martin (90.25 mg, 0.212 mmol, 1.1 equiv) and DCM (10 mL) at room temperature. The mixture was stirred 1 h at room temperature under nitrogen atmosphere. After the reaction, the mixture was concentrated under reduced pressure to afford (2S,5S)-5-((3-[(3-chloro-2-methoxyphenyl)amino]-2-(3-fluoropyridin-4-yl)-4-oxo-1H,5H,6H,7H-pyrrolo[3,2-c]pyridin-7-yl)methyl)-1,4-dioxane-2-carbaldehyde (95 mg, 95.37%) as a yellow solid.

LC-MS: (M+H)⁺ found: 515.

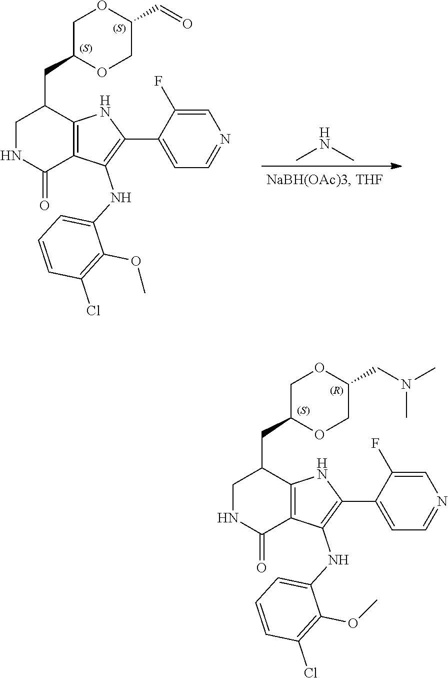

To a stirred mixture of (2S,5S)-5-({3-[(3-chloro-2-methoxyphenyl)amino]-2-(3-fluoropyridin-4-yl)-4-oxo-1H,5H,6H,7H-pyrrolo[3,2-c]pyridin-7-yl}methyl)-1,4-dioxane-2-carbaldehyde (50 mg, 0.097 mmol, 1 equiv) and dimethylamine (5.25 mg, 0.116 mmol, 1.2 equiv) in THF (5 mL) was added NaBH(OAc)3 (20.58 mg, 0.097 mmol, 1 equiv) dropwise at −20° C. under nitrogen atmosphere. The resulting mixture was stirred for 2 h at −20° C. Desired product could be detected by LCMS. The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (2:3) to afford 3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (30 mg, 56.79%) as a light yellow oil.

LC-MS: (M+H)⁺ found: 544.

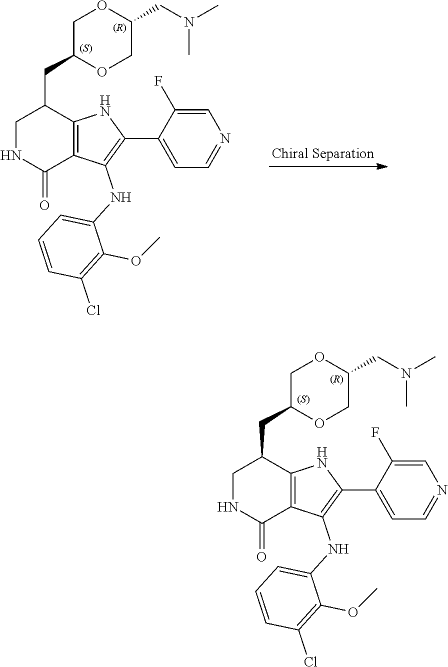

3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (40 mg, 0.074 mmol, 1 equiv) was used for chiral separation (Column: CHIRALPAK IG, 2*25 cm, 5 μm; Mobile Phase A: Hex:DCM=3:1 (0.5% 2M NH3-MeOH)-HPLC, Mobile Phase B: IPA-HPLC; Flow rate: 20 mL/min; Gradient: 20% B to 20% B in 19 min; Wave Length: 220/254 nm; RT1 (min): 7.92; RT2 (min): 9.81; Sample Solvent: ETOH: DCM=1:1; Injection Volume: 0.8 mL; Number Of Runs: 4), and 2 peak was splitted from the material, the postpeak is the product (7R*)-3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (5 mg, 12.50%). This resulted in (7R*)-3-[(3-chloro-2-methoxyphenyl)amino]-7-[(2S,5R)-5-[(dimethylamino)methyl]-1,4-dioxan-2-yl]methyl)-2-(3-fluoropyridin-4-yl)-1H,5H,6H,7H-pyrrolo[3,2-c]pyridin-4-one (5 mg, 12.50%) as a yellow solid.

LC-MS: (M+H)⁺ found: 544.

1H NMR (400 MHz, Methanol-d4) δ 8.44 (d, J=3.8 Hz, 1H), 8.16 (d, J=5.4 Hz, 1H), 7.52 (dd, J=7.0, 5.3 Hz, 1H), 6.73 (dd, J=8.1, 1.6 Hz, 1H), 6.67 (t, J=8.1 Hz, 1H), 6.23 (dd, J=8.0, 1.6 Hz, 1H), 4.04-3.96 (m, 1H), 3.99 (s, 3H), 3.86 (dd, J=11.5, 2.5 Hz, 1H), 3.79 (td, J=8.5, 7.0, 4.3 Hz, 2H), 3.62-3.54 (m, 1H), 3.52-3.44 (m, 2H), 3.46-3.34 (m, 2H), 2.48-2.34 (m, 2H), 2.32 (s, 6H), 1.83-1.67 (m, 2H).

### Example 109. (7R*)-3-[(3-chloro-2-methoxyphenyl)amino]-7-{[(2R,5S)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (9.2 mg, 4.35%). This resulted in (7R*)-3-[(3-chloro-2-methoxyphenyl)amino]-7-[(2R,5S)-5-[(dimethylamino)methyl]-1,4-dioxan-2-yl]methyl-2-(3-fluoropyridin-4-yl)-1H,5H,6H,7H-pyrrolo[3,2-c]pyridin-4-one (compound 563)

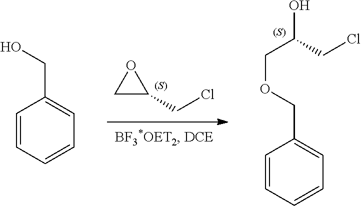

To a stirred solution/mixture of benzyl alcohol (1 g, 9.247 mmol, 1 equiv) and (−)-epichlorohydrin (0.94 g, 10.172 mmol, 1.1 equiv) in DCE (20 mL, 252.653 mmol, 27.32 equiv) was added BF3*Et₂O (0.13 g, 0.925 mmol, 0.1 equiv) dropwise at 0° C. under nitrogen atmosphere. The mixture was stirred for overnight at room temperature under nitrogen atmosphere. The reaction was quenched by the addition of Water (20 mL) at 0° C. The resulting mixture was extracted with EtOAc (3×20 mL). The combined organic layers dried over anhydrous Na2SO4. After filtration, the filtrate was concentrated under reduced pressure. The crude product was purified by reverse phase flash with the following conditions (ACN/H2O=3/7) to afford (2R)-1-(benzyloxy)-3-chloropropan-2-ol (800 mg, 43.11%) as a white solid.

LC-MS: (M+H)⁺ found: 201.

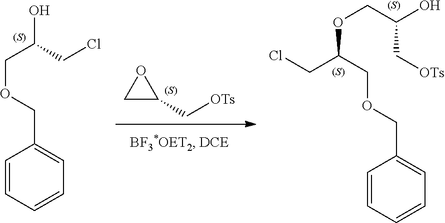

To a stirred solution of (2R)-1-(benzyloxy)-3-chloropropan-2-ol (72 g, 358.816 mmol, 1 equiv) and (2R)-oxiran-2-ylmethyl 4-methylbenzenesulfonate (24.57 g, 107.645 mmol, 0.3 equiv) in DCE (1.5 L) was added BF3*Et₂O (4.55 mL, 35.882 mmol, 0.1 equiv) dropwise at 0° C. under nitrogen atmosphere. The resulting mixture was stirred for overnight at room temperature under nitrogen atmosphere. The reaction was monitored by LCMS. The residue was washed with saturated sodium bicarbonate solution (2×1 L). The combined organic layers were dried over anhydrous Na2SO4. After filtration, the filtrate was concentrated under reduced pressure. The residue was purified by reverse flash chromatography with the following conditions: column, silica gel; mobile phase, MeCN in water, 40% to 50% gradient in 10 min; detector, UV 254 nm. This resulted in (2R)-1-{[(2R)-1-(benzyloxy)-3-chloropropan-2-yl]oxy}-3-[(4-methylbenzenesulfonyl)oxy]propan-2-ol (34 g, 22.09%) as a colorless oil.

LC-MS: (M+H)⁺ found: 429.

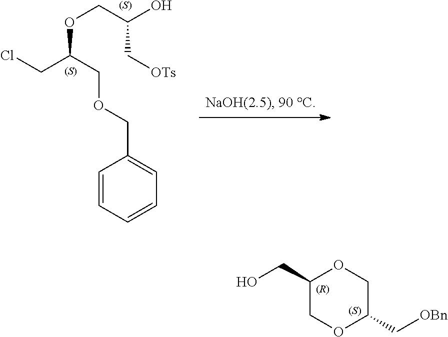

A solution of (2R)-1-{[(2R)-1-(benzyloxy)-3-chloropropan-2-yl]oxy}-3-[(4-methylbenzenesulfonyl)oxy]propan-2-ol (34 g, 79.269 mmol, 1 equiv) in NaOH (130 mL, 1.5N) was stirred for overnight at room temperature under nitrogen atmosphere. Then it was heated to 90° C. stirred for 4 h under nitrogen atmosphere. After that the reaction was cooled to room temperature and stirred for overnight. The mixture was then re-heated to 90° C. for about 2 h and finally cooled to room temperature. The mixture was acidified to 15 pH 6 with 1N HCl (aq.). The resulting mixture was extracted with EtOAc (3×mL). The combined organic layers were washed with brine (2×2 mL), dried over anhydrous Na2SO4. After filtration, the filtrate was concentrated under reduced pressure. The residue was purified by reverse flash chromatography with the following conditions: column, silica gel; mobile phase, MeCN in water, 50% to 60% gradient in 10 min; detector, UV 254 nm. This resulted in [(2S,5R)-5-[(benzyloxy)methyl]-1,4-dioxan-2-yl]methanol (6. 1 g, 32.29%) as a colorless oil.

LC-MS: (M+H)⁺ found: 239.

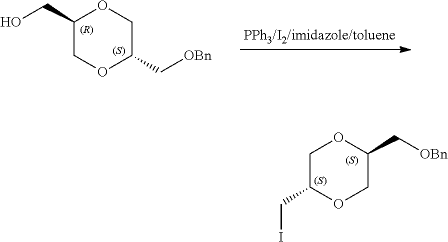

To a stirred solution of [(2S,5R)-5-[(benzyloxy)methyl]-1,4-dioxan-2-yl]methanol (1 g, 4.197 mmol, 1 equiv) and Imidazole (0.57 g, 8.394 mmol, 2 equiv) in Toluene (8 mL) were added PPh₃ (1.10 g, 4.197 mmol, 1 equiv) and iodine (1.07 g, 4.197 mmol, 1 equiv) in portions at room temperature under nitrogen atmosphere. The resulting mixture was stirred for 1 h at room temperature under nitrogen atmosphere. To the above mixture was added THF (4 mL) dropwise at room temperature. The resulting mixture was stirred for additional 1 h at room temperature. The reaction was monitored by LCMS. The reaction was quenched by the addition of sat. sodium hyposulfite (aq.) (5 mL) at room temperature. The resulting mixture was extracted with EtOAc (3×20 mL). The combined organic layers were washed with brine (2×2 mL), dried over anhydrous Na2SO4. After filtration, the filtrate was concentrated under reduced pressure. The residue was purified by silica gel column chromatography, eluted with PE/EA (10:1) to afford (2R,5R)-2-[(benzyloxy)methyl]-5-(iodomethyl)-1,4-dioxane (1.3 g, 88.97%) as a colorless oil.

LC-MS: (M+H)⁺ found: 349.

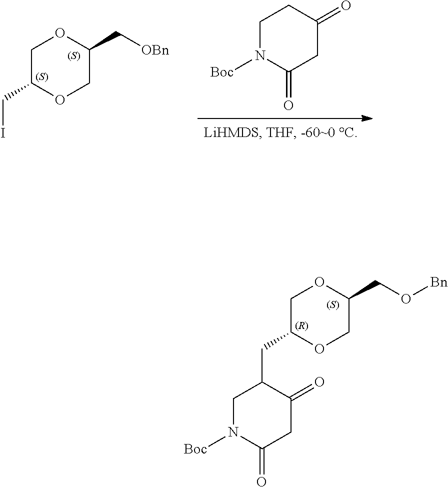

To a stirred mixture of (2R,5R)-2-[(benzyloxy)methyl]-5-(iodomethyl)-1,4-dioxane (1 g, 2.872 mmol, 1 equiv) and tert-butyl 2,4-dioxopiperidine-1-carboxylate (0.73 g, 3.446 mmol, 1.2 equiv) in THF (20 mL) was added LiHMDS (1.44 g, 8.616 mmol, 3 equiv) dropwise at −20° C. under nitrogen atmosphere. The resulting mixture was stirred for 2 h at 20 −20° C. Desired product could be detected by LCMS. The mixture was acidified to pH 5 with HCl (aq.). The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (4:1) to afford tert-butyl 5-{[(2S,5R)-5-[(benzyloxy)methyl]-1,4-dioxan-2-yl]methyl}-2,4-dioxopiperidine-1-carboxylate (350 mg, 28.11%) as a light green oil.

LC-MS: (M+H)⁺ found: 434.

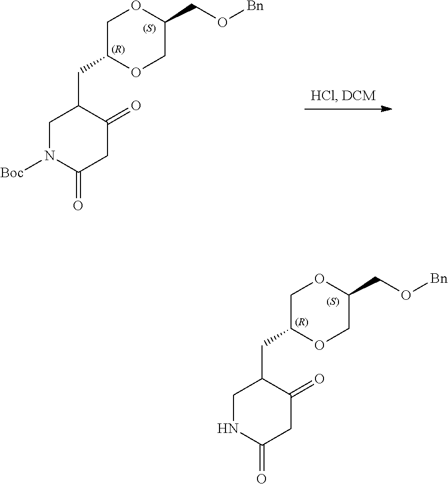

Into a 100 mL round-bottom flask were added tert-butyl 5-{[(2S,5R)-5-[(benzyloxy)methyl]-1,4-dioxan-2-yl]methyl}-2,4-dioxopiperidine-1-carboxylate (1 g, 2.307 mmol, 1 equiv), HCl (gas) in 1,4-dioxane (10 mL) and DCM (20 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:2) to afford 5-{[(2S,5R)-5-[(benzyloxy)methyl]-1,4-dioxan-2-yl]methyl}piperidine-2,4-dione (650 mg, 84.52%) as a yellow solid.

LC-MS: (M+H)⁺ found: 334.

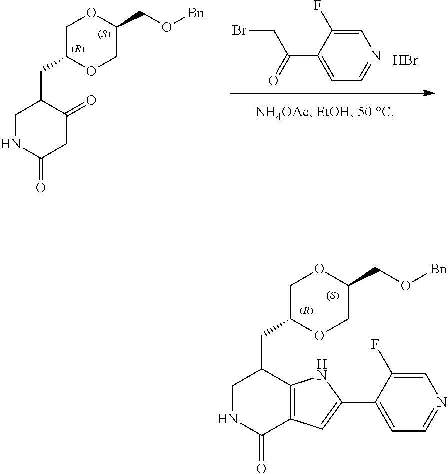

Into a 100 mL round-bottom flask were added 5-{[(2S,5R)-5-[(benzyloxy)methyl]-1,4-dioxan-2-yl]methyl}piperidine-2,4-dione (500 mg, 1.500 mmol, 1 equiv), 2-bromo-1-(3-fluoropyridin-4-yl)ethanone (490.48 mg, 2.250 mmol, 1.5 equiv), NH4OAc (1156.07 mg, 15.000 mmol, 10 equiv) and EtOH (10 mL) at room temperature. The mixture was stirred for 5 h at 50 degrees C. under nitrogen atmosphere. After the reaction, The resulting mixture was concentrated under reduced pressure. The residue was purified by silica gel column chromatography, eluted with PE/EtOAc (1:1) to afford 7-{[(2S,5R)-5-[(benzyloxy)methyl]-1,4-dioxan-2-yl]methyl}-2-(3-fluoropyridin-4-yl)-1H,5H,6H,7H-pyrrolo[3,2-c]pyridin-4-one (400 mg, 59.07%) as a yellow solid.

LC-MS: (M+H)⁺ found: 452.

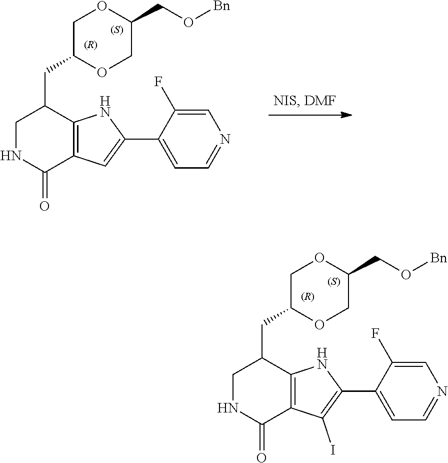

To a stirred solution of 7-{[(2S,5R)-5-[(benzyloxy)methyl]-1,4-dioxan-2-yl]methyl}-2-(3-fluoropyridin-4-yl)-1H,5H,6H,7H-pyrrolo[3,2-c]pyridin-4-one (500 mg, 1.107 mmol, 1 equiv) in DMF (10 mL) was added NIS (298.98 mg, 1.328 mmol, 1.2 equiv) in portions at −35° C. under nitrogen atmosphere. The reaction solution was stirred at room temperature for Ih, The resulting mixture was extracted with EtOAc (4×40 mL). The combined organic layers were washed with brine (1×20 mL), dried over anhydrous Na2SO4. After filtration, the filtrate was concentrated under reduced pressure. The residue was purified by Prep-TLC (PE/EtOAc 1:1) to afford 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (420 mg, 65.68%) as a white solid.

LC-MS: (M+H)⁺ found: 578.

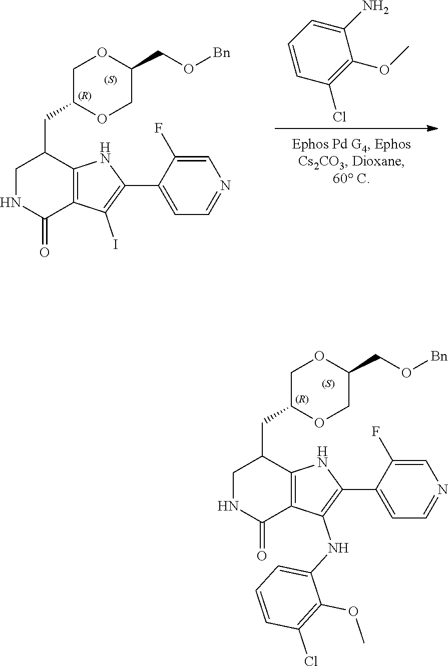

To a solution of 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (400 mg, 0.693 mmol, 1 equiv) and Cs₂CO₃ (451.43 mg, 1.386 mmol, 2 equiv) in DMF (10 mL) were added EPhos Pd G4 (63.64 mg, 0.069 mmol, 0.1 equiv) and 3-chloro-2-methoxyaniline (131.02 mg, 0.832 mmol, 1.2 equiv) at room temperature under argon atmosphere. The resulting mixture was stirred for 4 h at 50 degrees C. under argon atmosphere. The resulting mixture was concentrated under reduced pressure. The residue was purified by silica gel column chromatography, eluted with CH₂Cl₂/MeOH (30:1) to afford 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (320 mg, 76.09%) as a yellow solid.

LC-MS: (M+H)⁺ found: 607.

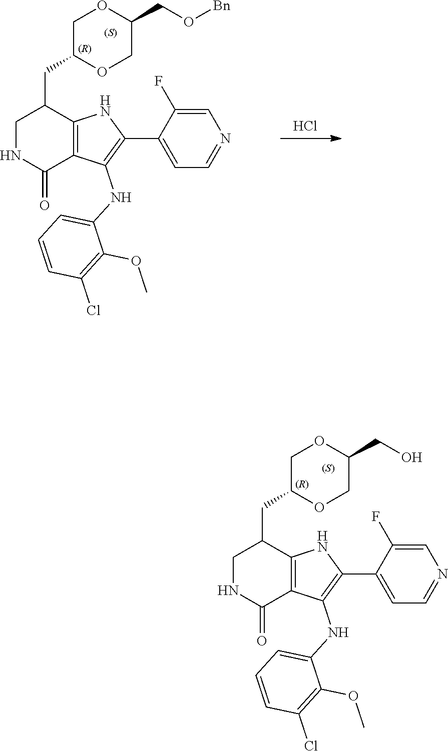

Into a 100 mL round-bottom flask were added 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (100 mg, 0.165 mmol, 1 equiv) and HCl (gas) in 1,4-dioxane (10 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:4) to afford 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (80 mg, 93.95%) as a yellow solid.

LC-MS: (M+H)⁺ found: 517.

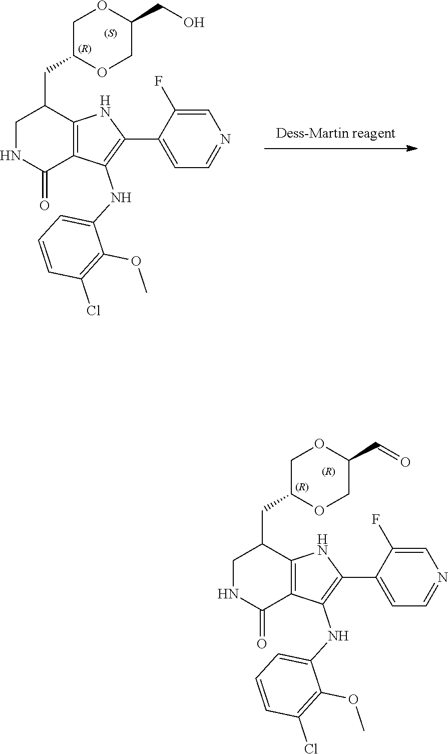

Into a 50 mL round-bottom flask were added 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (100 mg, 0.193 mmol, 1 equiv), Dess-Martin (90.25 mg, 0.212 mmol, 1.1 equiv) and DCM (10 mL) at room temperature. The mixture was stirred 1 h at room temperature under nitrogen atmosphere. After the reaction, the mixture was concentrated under reduced pressure to afford (2S,5S)-5-({3-[(3-chloro-2-methoxyphenyl)amino]-2-(3-fluoropyridin-4-yl)-4-oxo-1H,5H,6H,7H-pyrrolo[3,2-c]pyridin-7-yl}methyl)-1,4-dioxane-2-carbaldehyde (95 mg, 95.37%) as a yellow solid.

LC-MS: (M+H)⁺ found: 515.

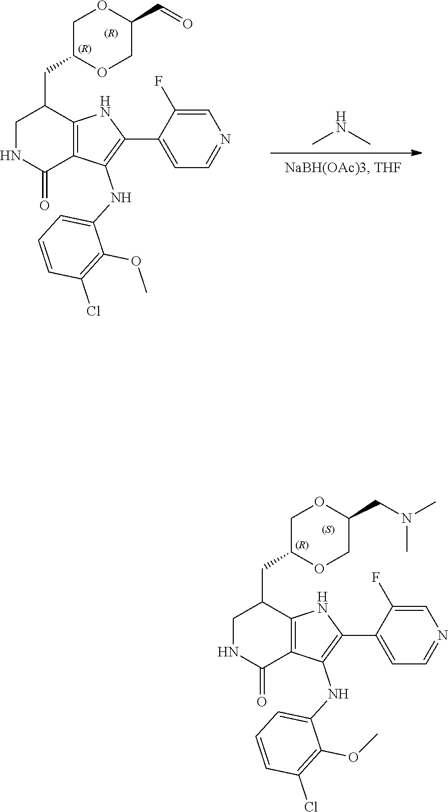

To a stirred mixture of (2S,5S)-5-((3-[(3-chloro-2-methoxyphenyl)amino]-2-(3-fluoropyridin-4-yl)-4-oxo-1H,5H,6H,7H-pyrrolo[3,2-c]pyridin-7-yl)methyl)-1,4-dioxane-2-carbaldehyde (50 mg, 0.097 mmol, 1 equiv) and dimethylamine (5.25 mg, 0.116 mmol, 1.2 equiv) in THF (5 mL) was added NaBH(OAc)3 (20.58 mg, 0.097 mmol, 1 equiv) dropwise at −20° C. under nitrogen atmosphere. The resulting mixture was stirred for 2 h at −20° C. Desired product could be detected by LCMS. The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (2:3) to afford 3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (30 mg, 56.79%) as a light yellow oil.

LC-MS: (M+H)⁺ found: 544.

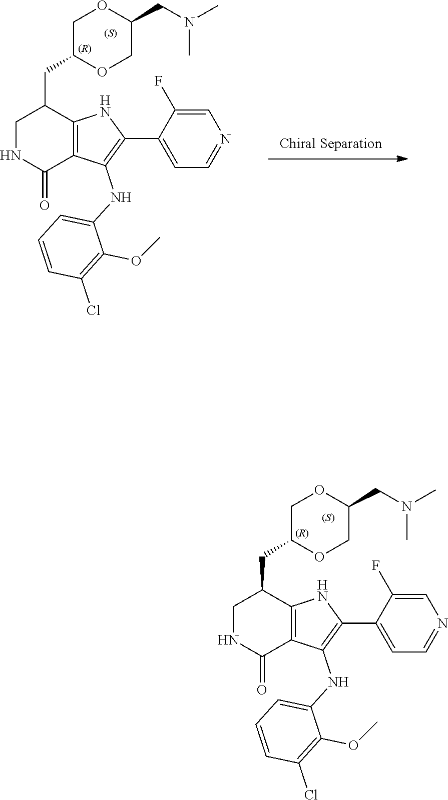

(2R,5R)-5-({3-[(3-chloro-2-methoxyphenyl)amino]-2-(3-fluoropyridin-4-yl)-4-oxo-1H,5H,6H,7H-pyrrolo[3,2-c]pyridin-7-yl}methyl)-1,4-dioxane-2-carbaldehyde (200 mg, 0.388 mmol, 1 equiv) was used for chiral separation (Column: CHIRALPAK IG, 2*25 cm, 5 μm; Mobile Phase A: Hex:DCM=3:1 (0.5% 2M NH3-MeOH)-HPLC, Mobile Phase B: IPA-HPLC; Flow rate: 20 mL/min; Gradient: 20% B to 20% B in 19 min; Wave Length: 220/254 nm; RT1 (min): 7.92; RT2 (min): 9.81; Sample Solvent: ETOH:DCM=1:1; Injection Volume: 0.8 mL; Number Of Runs: 4), and 2 peak was splitted from the material, the prepeak is the product (7R*)-3-[(3-chloro-2-methoxyphenyl)amino]-7-{[(2R,5S)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (9.2 mg, 4.35%). This resulted in (7R*)-3-[(3-chloro-2-methoxyphenyl)amino]-7-{[(2R,5S)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (9.2 mg, 4.35%) as a yellow solid.

LC-MS: (M+H)⁺ found: 544.

¹H NMR (400 MHz, Methanol-d4) δ 8.43 (d, J=3.5 Hz, 1H), 8.18 (dd, J=5.2, 1.0 Hz, 1H), 7.52 (dd, J=6.9, 5.2 Hz, 1H), 6.71 (dd, J=8.0, 1.6 Hz, 1H), 6.65 (t, J=8.0 Hz, 1H), 6.23 (dd, J=8.0, 1.6 Hz, JH), 3.98 (s, 3H), 3.91 (dd, J=110.5, 2.6 Hz, 1H), 3.82-3.68 (m, 3H), 3.61 (t, J=9.2 Hz, 1H), 3.49-3.37 (m, 3H), 3.30-3.21 (m, 1H), 2.43 (dd, J=13.1, 7.6 Hz, 1H), 2.35 (d, J=3.7 Hz, 1H), 2.32 (s, 6H), 1.87 (ddd, J=14.3, 7.6, 3.6 Hz, J H), 1.75 (ddd, J=14.6, 8.4, 6.7 Hz, 1H).

### Example 110. (7R*)-3-[(3-chloro-2-methoxyphenyl)amino]-7-{[(2R,5S)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (14.9 mg, 7.05%). This resulted in (7R*)-3-[(3-chloro-2-methoxyphenyl)amino]-7-{[(2R,5S)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (compound 562)

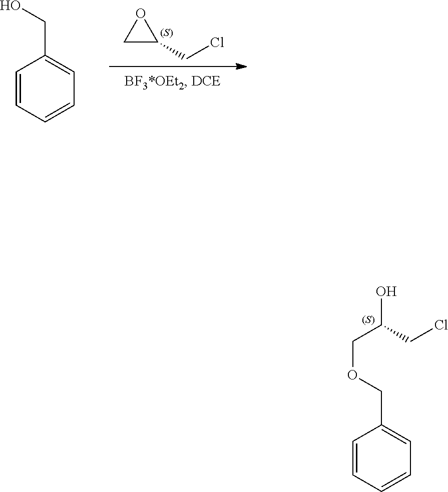

To a stirred solution/mixture of benzyl alcohol (1 g, 9.247 mmol, 1 equiv) and (−)-epichlorohydrin (0.94 g, 10.172 mmol, 1.1 equiv) in DCE (20 mL, 252.653 mmol, 27.32 equiv) was added BF3*Et2O (0.13 g, 0.925 mmol, 0.1 equiv) dropwise at 0° C. under nitrogen atmosphere. The mixture was stirred for overnight at room temperature under nitrogen atmosphere. The reaction was quenched by the addition of Water (20 mL) at 0° C. The resulting mixture was extracted with EtOAc (3×20 mL). The combined organic layers dried over anhydrous Na2SO4. After filtration, the filtrate was concentrated under reduced pressure. The crude product was purified by reverse phase flash with the following conditions (ACN/H2O=3/7) to afford (2R)-1-(benzyloxy)-3-chloropropan-2-ol (800 mg, 43.11%) as a white solid.

LC-MS: (M+H)⁺ found: 201.

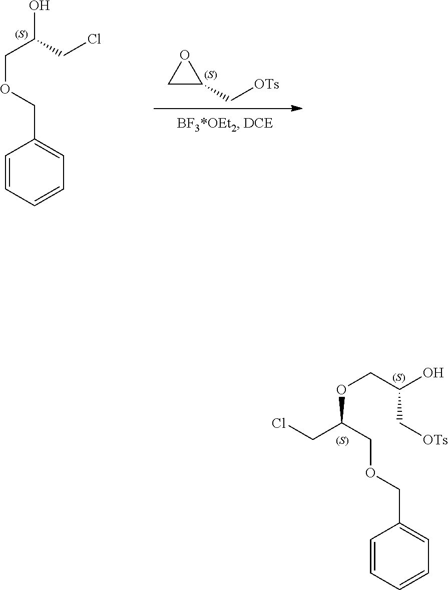

To a stirred solution of (2R)-1-(benzyloxy)-3-chloropropan-2-ol (72 g, 358.816 mmol, 1 equiv) and (2R)-oxiran-2-ylmethyl 4-methylbenzenesulfonate (24.57 g, 107.645 mmol, 0.3 equiv) in DCE (1.5 L) was added BF3*Et2O (4.55 mL, 35.882 mmol, 0.1 equiv) dropwise at 0° C. under nitrogen atmosphere. The resulting mixture was stirred for overnight at room temperature under nitrogen atmosphere. The reaction was monitored by LCMS. The residue was washed with saturated sodium bicarbonate solution (2×1 L). The combined organic layers were dried over anhydrous Na2SO4. After filtration, the filtrate was concentrated under reduced pressure. The residue was purified by reverse flash chromatography with the following conditions: column, silica gel; mobile phase, MeCN in water, 40% to 50% gradient in 10 min; detector, UV 254 nm. This resulted in (2R)-1-{[(2R)-1-(benzyloxy)-3-chloropropan-2-yl]oxy}-3-[(4-methylbenzenesulfonyl)oxy]propan-2-ol (34 g, 22.09%) as a colorless oil.

LC-MS: (M+H)⁺ found: 429.

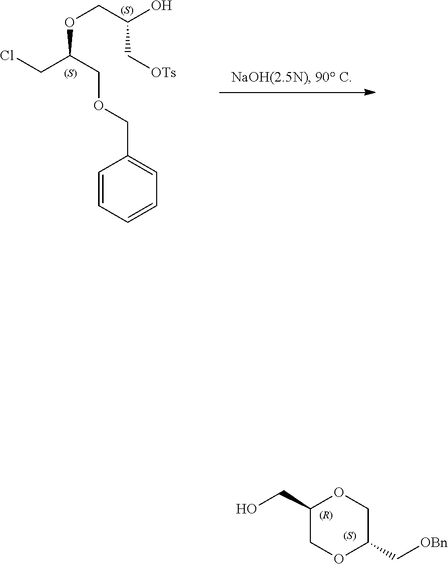

A solution of (2R)-1-{[(2R)-1-(benzyloxy)-3-chloropropan-2-yl]oxy)-3-[(4-methylbenzenesulfonyl)oxy]propan-2-ol (34 g, 79.269 mmol, 1 equiv) in NaOH (130 mL, 1.5N) was stirred for overnight at room temperature under nitrogen atmosphere. Then it was heated to 90° C. stirred for 4 h under nitrogen atmosphere. After that the reaction was cooled to room temperature and stirred for overnight. The mixture was then re-heated to 90° C. for about 2 h and finally cooled to room temperature. The mixture was acidified to pH 6 with 1N HCl (aq.). The resulting mixture was extracted with EtOAc (3×mL). The combined organic layers were washed with brine (2×2 mL), dried over anhydrous Na₂SO₄. After filtration, the filtrate was concentrated under reduced pressure. The residue was purified by reverse flash chromatography with the following conditions: column, silica gel; mobile phase, MeCN in water, 50% to 60% gradient in 10 min; detector, UV 254 nm. This resulted in [(2S,5R)-5-[(benzyloxy)methyl]-1,4-dioxan-2-yl]methanol (6.1 g, 32.29%) as a colorless oil.

LC-MS: (M+H)⁺ found: 239.

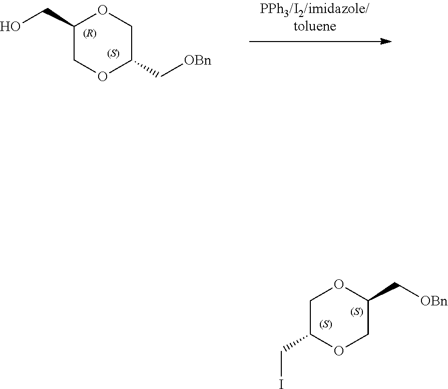

To a stirred solution of [(2S,5R)-5-[(benzyloxy)methyl]-1,4-dioxan-2-yl]methanol (1 g, 4.197 mmol, 1 equiv) and Imidazole (0.57 g, 8.394 mmol, 2 equiv) in Toluene (8 mL) were added PPh₃ (1.10 g, 4.197 mmol, 1 equiv) and iodine (1.07 g, 4.197 mmol, 1 equiv) in portions at room temperature under nitrogen atmosphere. The resulting mixture was stirred for 1 h at room temperature under nitrogen atmosphere. To the above mixture was added THF (4 mL) dropwise at room temperature. The resulting mixture was stirred for additional 1 h at room temperature. The reaction was monitored by LCMS. The reaction was quenched by the addition of sat. sodium hyposulfite (aq.) (5 mL) at room temperature. The resulting mixture was extracted with EtOAc (3×20 mL). The combined organic layers were washed with brine (2×2 mL), dried over anhydrous Na2SO4. After filtration, the filtrate was concentrated under reduced pressure. The residue was purified by silica gel column chromatography, eluted with PE/EA (10:1) to afford (2R,5R)-2-[(benzyloxy)methyl]-5-(iodomethyl)-1,4-dioxane (1.3 g, 88.97%) as a colorless oil.

LC-MS: (M+H)⁺ found: 349.

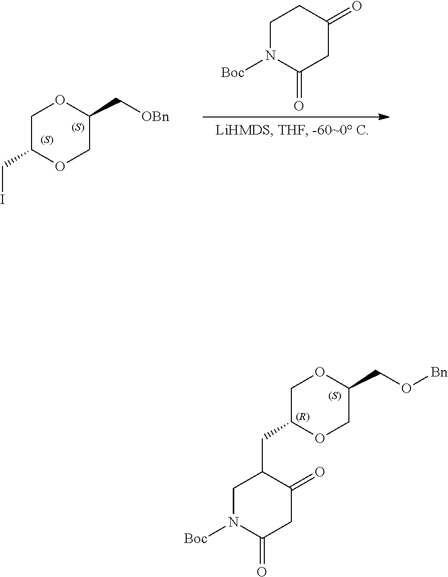

To a stirred mixture of (2R,5R)-2-[(benzyloxy)methyl]-5-(iodomethyl)-1,4-dioxane (1 g, 2.872 mmol, 1 equiv) and tert-butyl 2,4-dioxopiperidine-1-carboxylate (0.73 g, 3.446 mmol, 1.2 equiv) in THF (20 mL) was added LiHMDS (1.44 g, 8.616 mmol, 3 equiv) dropwise at −20° C. under nitrogen atmosphere. The resulting mixture was stirred for 2 h at −20° C. Desired product could be detected by LCMS. The mixture was acidified to pH 5 with HCl (aq.). The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (4:1) to afford tert-butyl 5-{[(2S,5R)-5-[(benzyloxy)methyl]-1,4-dioxan-2-yl]methyl}-2,4-dioxopiperidine-1-carboxylate (350 mg, 28.11%) as a light green oil.

LC-MS: (M+H)⁺ found: 434.

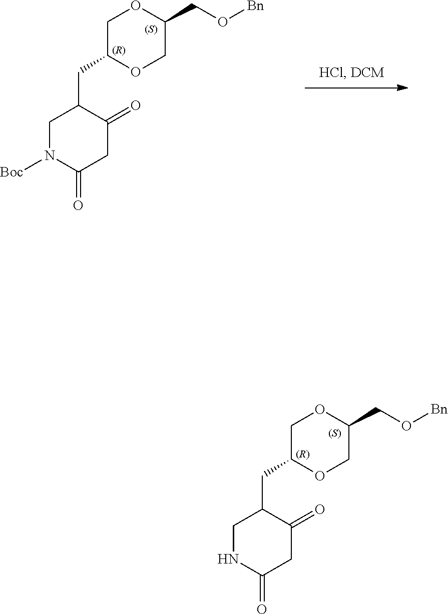

Into a 100 mL round-bottom flask were added tert-butyl 5-{[(2S,5R)-5-[(benzyloxy)methyl]-1,4-dioxan-2-yl]methyl}-2,4-dioxopiperidine-1-carboxylate (1 g, 2.307 mmol, 1 equiv), HCl (gas) in 1,4-dioxane (10 mL) and DCM (20 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:2) to afford 5-{[(2S,5R)-5-[(benzyloxy)methyl]-1,4-dioxan-2-yl]methyl}piperidine-2,4-dione (650 mg, 84.52%) as a yellow solid.

LC-MS: (M+H)⁺ found: 334.

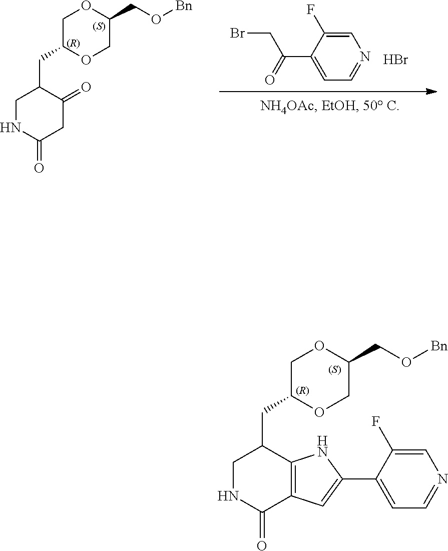

Into a 100 mL round-bottom flask were added 5-{[(2S,5R)-5-[(benzyloxy)methyl]-1,4-dioxan-2-yl]methyl}piperidine-2,4-dione (500 mg, 1.500 mmol, 1 equiv), 2-bromo-1-(3-fluoropyridin-4-yl)ethanone (490.48 mg, 2.250 mmol, 1.5 equiv), NH₄OAc (1156.07 mg, 15.000 mmol, 10 equiv) and EtOH (10 mL) at room temperature. The mixture was stirred for 5 h at 50 degrees C. under nitrogen atmosphere. After the reaction, The resulting mixture was concentrated under reduced pressure. The residue was purified by silica gel column chromatography, eluted with PE/EtOAc (1:1) to afford 7-{[(2S,5R)-5-[(benzyloxy)methyl]-1,4-dioxan-2-yl]methyl}-2-(3-fluoropyridin-4-yl)-1H,5H,6H,7H-pyrrolo[3,2-c]pyridin-4-one (400 mg, 59.07%) as a yellow solid.

LC-MS: (M+H)⁺ found: 452.

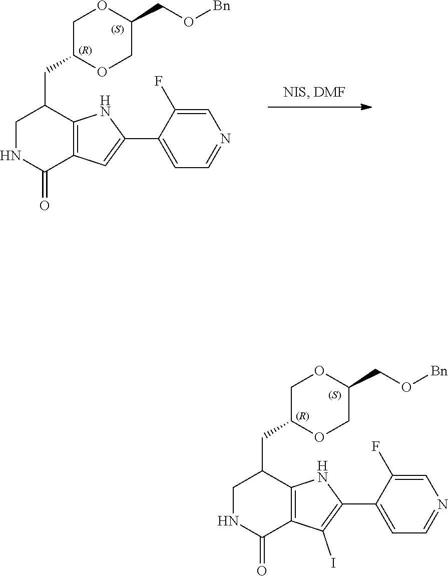

To a stirred solution of 7-{[(2S,5R)-5-[(benzyloxy)methyl]-1,4-dioxan-2-yl]methyl}-2 (3-fluoropyridin-4-yl)-1H,5H,6H,7H-pyrrolo[3,2-c]pyridin-4-one (500 mg, 1.107 mmol, 1 equiv) in DMF (10 mL) was added NIS (298.98 mg, 1.328 mmol, 1.2 equiv) in portions at −35° C. under nitrogen atmosphere. The reaction solution was stirred at room temperature for 1 h, The resulting mixture was extracted with EtOAc (4×40 mL). The combined organic layers were washed with brine (1×20 mL), dried over anhydrous Na2SO4. After filtration, the filtrate was concentrated under reduced pressure. The residue was purified by Prep-TLC (PE/EtOAc 1:1) to afford 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (420 mg, 65.68%) as a white solid.

LC-MS: (M+H)⁺ found: 578.

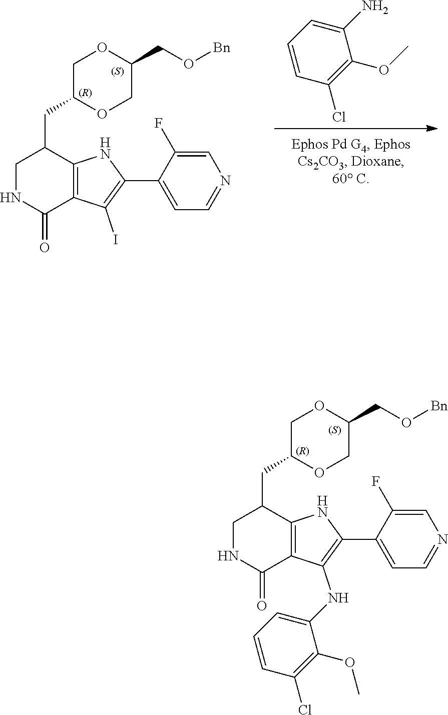

To a solution of 7-{[(2S,5R)-5-[(benzyloxy)methyl]-1,4-dioxan-2-yl]methyl}-2-(3-fluoropyridin-4-yl)-3-iodo-1H,5H,6H,7H-pyrrolo[3,2-c]pyridin-4-one (400 mg, 0.693 mmol, 1 equiv) and Cs₂CO₃ (451.43 mg, 1.386 mmol, 2 equiv) in DMF (10 mL) were added EPhos Pd G4 (63.64 mg, 0.069 mmol, 0.1 equiv) and 3-chloro-2-methoxyaniline (131.02 mg, 0.832 mmol, 1.2 equiv) at room temperature under argon atmosphere. The resulting mixture was stirred for 4 h at 50 degrees C. under argon atmosphere. The resulting mixture was concentrated under reduced pressure. The residue was purified by silica gel column chromatography, eluted with CH2Cl2/MeOH (30:1) to afford 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (320 mg, 76.09%) as a yellow solid.

LC-MS: (M+H)⁺ found: 607.

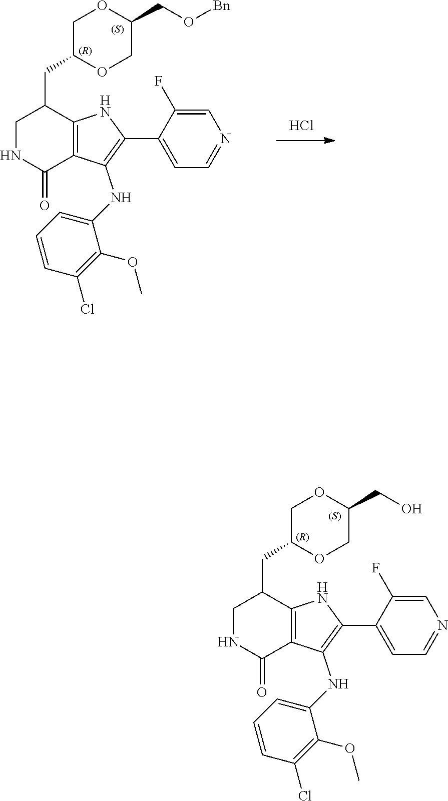

Into a 100 mL round-bottom flask were added 7-{[(2S,5R)-5-[(benzyloxy)methyl]-1,4-dioxan-2-yl]methyl}-3-[(3-chloro-2-methoxyphenyl)amino]-2-(3-fluoropyridin-4-yl)-1H,5H,6H,7H-pyrrolo[3,2-c]pyridin-4-one (100 mg, 0.165 mmol, 1 equiv) and HCl (gas) in 1,4-dioxane (10 mL) at room temperature. The mixture was stirred 1 h at room temperature under nitrogen atmosphere. After the reaction, the mixture was concentrated under reduced pressure. The residue was purified by silica gel column chromatography, eluted with PE/EtOAc (1:4) to afford 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (80 mg, 93.95%) as a yellow solid.

LC-MS: (M+H)⁺ found: 517.

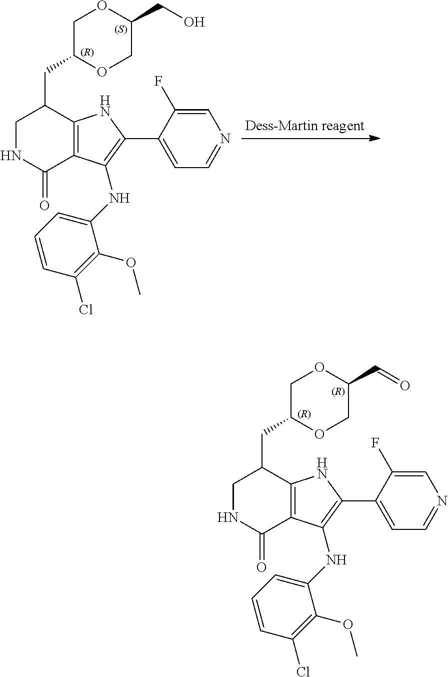

Into a 50 mL round-bottom flask were added 3-[(3-chloro-2-methoxyphenyl)amino]-2-(3-fluoropyridin-4-yl)-7-{[(2S,5R)-5-(hydroxymethyl)-1,4-dioxan-2-yl]methyl}-1H,5H,6H,7H-pyrrolo[3,2-c]pyridin-4-one (100 mg, 0.193 mmol, 1 equiv), Dess-Martin (90.25 mg, 0.212 mmol, 1.1 equiv) and DCM (10 mL) at room temperature. The mixture was stirred 1 h at room temperature under nitrogen atmosphere. After the reaction, the mixture was concentrated under reduced pressure to afford (2S,5S)-5-((3-[(3-chloro-2-methoxyphenyl)amino]-2-(3-fluoropyridin-4-yl)-4-oxo-1H,5H,6H,7H-pyrrolo[3,2-c]pyridin-7-yl)methyl)-1,4-dioxane-2-carbaldehyde (95 mg, 95.37%) as a yellow solid.

LC-MS: (M+H)⁺ found: 515.

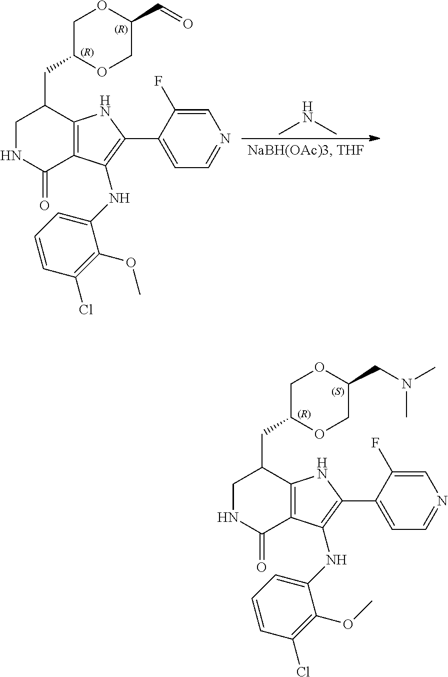

To a stirred mixture of (2S,5S)-5-({3-[(3-chloro-2-methoxyphenyl)amino]-2-(3-fluoropyridin-4-yl)-4-oxo-1H,5H,6H,7H-pyrrolo[3,2-c]pyridin-7-yl}methyl)-1,4-dioxane-2-carbaldehyde (50 mg, 0.097 mmol, 1 equiv) and dimethylamine (5.25 mg, 0.116 mmol, 1.2 equiv) in THF (5 mL) was added NaBH(OAc)3 (20.58 mg, 0.097 mmol, 1 equiv) dropwise at −20° C. under nitrogen atmosphere. The resulting mixture was stirred for 2 h at −20° C. Desired product could be detected by LCMS. The resulting mixture was extracted with EtOAc (2×40 mL). The combined organic layers were washed with brine (2×100 mL), dried over anhydrous Na2SO4. After filtration, the filtrate was concentrated under reduced pressure. The residue was purified by silica gel column chromatography, eluted with PE/EA (2:3) to afford 3-[(3-chloro-2-methoxyphenyl)amino]-7-{[(2S,5R)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (30 mg, 56.79%) as a light yellow oil.

LC-MS: (M+H)⁺ found: 544.

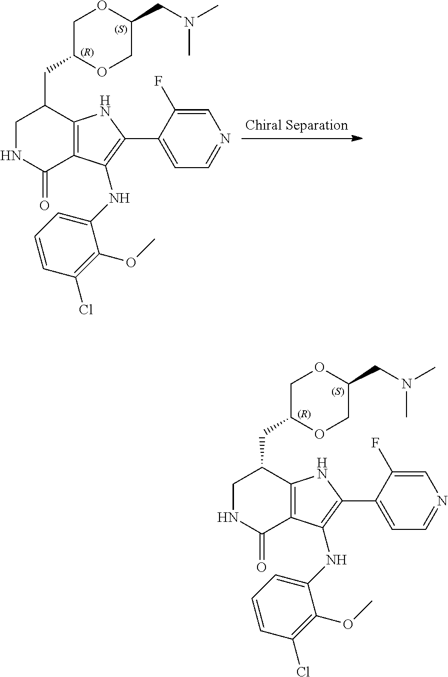

(2R,5R)-5-({3-[(3-chloro-2-methoxyphenyl)amino]-2-(3-fluoropyridin-4-yl)-4-oxo-1H,5H,6H,7H-pyrrolo[3,2-c]pyridin-7-yl}methyl)-1,4-dioxane-2-carbaldehyde (200 mg, 0.388 mmol, 1 equiv) was used for chiral separation (Column: CHIRALPAK IG, 2*25 cm, 5 μm; Mobile Phase A: Hex:DCM=3:1 (0.5% 2M NH3-MeOH)-HPLC, Mobile Phase B: IPA-HPLC; Flow rate: 20 mL/min; Gradient: 20% B to 20% B in 19 min; Wave Length: 220/254 nm; RT1 (min): 7.92: RT2 (min): 9.81; Sample Solvent: ETOH:DCM=1:1; Injection Volume: 0.8 mL; Number Of Runs: 4), and 2 peak was splitted from the material, the postpeak is the product (7R*)-3-[(3-chloro-2-methoxyphenyl)amino]-7-{[(2R,5S)-5-[(dimethylamino)methyl]-1,4-dioxan-2-yl]methyl}-2-(3-fluoropyridin-4-yl)-1H,5H,6H,7H-pyrrolo[3,2-c]pyridin-4-one (14.9 mg, 7.05%). This resulted in (7R*)-3-[(3-chloro-2-methoxyphenyl)amino]-7-{[(2R,5S)-5-[(dimethylamino)methyl]-1,4-dioxan-2-yl]methyl)-2-(3-fluoropyridin-4-yl)-1H,5H,6H,7H-pyrrolo[3,2-c]pyridin-4-one (14.9 mg, 7.05%) as a yellow solid.

LC-MS: (M+H)⁺ found: 544.

¹H NMR (400 MHz, Methanol-d4) δ 8.44 (d, J=3.9 Hz, 1H), 8.16 (dd, J=5.3, 1.1 Hz, 1H), 7.52 (dd, J=7.1, 5.3 Hz, 1H), 6.73 (dd, J=8.1, 1.6 Hz, 1H), 6.67 (t, J=8.0 Hz, 1H), 6.23 (dd, J=8.0, 1.6 Hz, 1H), 4.04-3.96 (m, 1H), 3.99 (s, 3H), 3.90-3.75 (m, 2H), 3.80 (s, 1H), 3.64-3.54 (m, 1H), 3.52-3.33 (m, 3H), 3.31 (s, 1H), 2.47 (dd, J=13.1, 7.6 Hz, 1H), 2.43-2.36 (m, 1H), 2.35 (s, 6H), 1.75 (s, 1H), 1.76-1.67 (m, 1H).

### Example 111. (7R)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (compound 570)

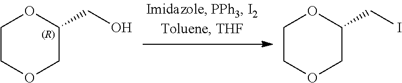

To a suspension of (R)-(1,4-dioxan-2-yl)methanol (100.00 g, 0.847 mol, 1.00 equiv) in Tolune (1000 mL) and THF (500 mL) was added imidazole (115.2 g, 1.69 mol, 2.00 equiv), PPh₃ (214.4 g, 0.847 mol, 1.00 equiv) at room temperature, I₂ (214.4 g, 0.847 mol, 1.00 equiv) was added at 0 degrees C. The mixture was stirred for 3 h at room temperature under nitrogen atmosphere.

Desired product could be detected by LCMS. The resulting solution was quenching with 500 ml of Na₂S₂O₃ saturated solution. The aqueous layer was extracted with EtOAc (3×250 mL), dried (Na₂SO₄) and concentrated. The resulting residue was purified on silica (EtOAc/PE=1:6) to provide 130 g (67%) of the title compound as a colorless oil.

LC-MS: (M+H)⁺ found: 229.

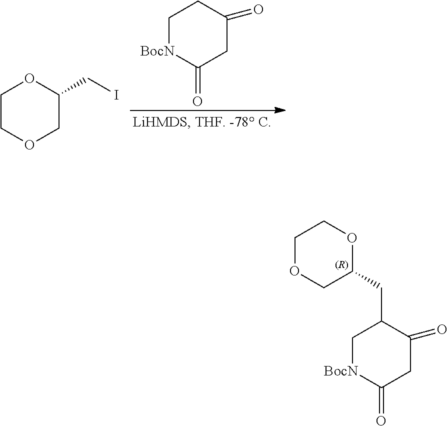

To a suspension of (S)-2-(iodomethyl)-1,4-dioxane (104.00 g, 0.457 mol, 1.40 equiv) and tert-butyl 2,4-dioxopiperidine-1-carboxylate (70.00 g, 0.327 mol, 1.00 equiv) in THF (1000 mL) was dropwise added LiHMDS (1.37 L, 1.37 mol, 3.00 equiv) at −60° C. The mixture was stirred for 1 h at 0 degrees under nitrogen atmosphere.

Desired product could be detected by LCMS. The resulting solution was quenching with 500 ml of HCl (5%) at 0 degrees. The aqueous layer was extracted with EtOAc (3×250 mL), dried (Na₂SO₄) and concentrated. The resulting residue was purified by silica gel column (PE/EtOAc, 1:1) to provide 45 g (46%) of the title compound as a light brown oil.

LC-MS: (M+H)⁺ found: 314.

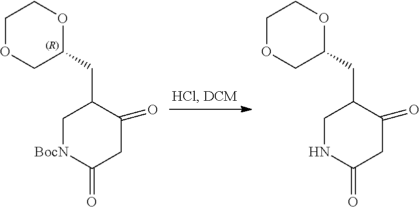

To a stirred mixture of tert-butyl 5-(((R)-1,4-dioxan-2-yl)methyl)-2,4-dioxopiperidine-1-carboxylate (45.00 g) in DCM (300 mL) was added HCl (150 mL) dropwise at 0 degrees C. under nitrogen atmosphere. The mixture was stirred at 0 degrees C. for 1 h. The resulting mixture was concentrated under reduced pressure to afford 30 g crude product, which was used to the next step without further purification.

LC-MS: (M+H)⁺ found: 214.

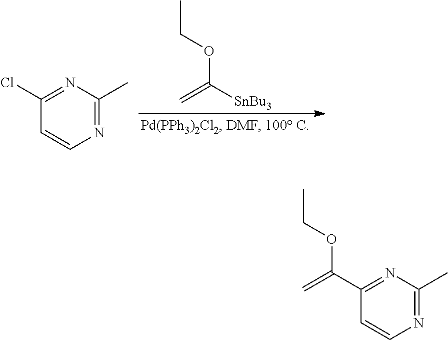

A mixture of 4-chloro-2-methylpyrimidine (20.00 g, 156.2 mmol, 1.00 equiv), tributyl(1-ethoxyvinyl)stannane (45.82 g, 468 mmol, 3.00 equiv) and Pd(PPh₃)₂Cl₂ (8.86 g, 15.6 mmol, 0.10 equiv) in DMF (200.00 mL) was stirred for 12 h at 100 degrees C. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EtOAc (5:1) to afford 4-(1-ethoxyvinyl)-2-methylpyrimidine 17 g (66%) as a yellow oil.

LC-MS: M+H found: 165.0.

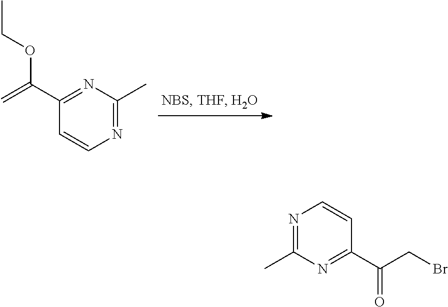

A mixture of 4-(1-ethoxyvinyl)-2-methylpyrimidine (17.00 g, 103.0 mmol, 1.00 equiv), NBS (30.13 g, 133.9 mmol, 1.30 equiv) and H₂O (15 mL) in THF (150 mL) was stirred for 1 h at room temperature. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. This resulted in 2-bromo-1-(2-methylpyrimidin-4-yl) ethan-1-one 18 g crude as a brown oil.

LC-MS: (M+H)⁺ found: 215.

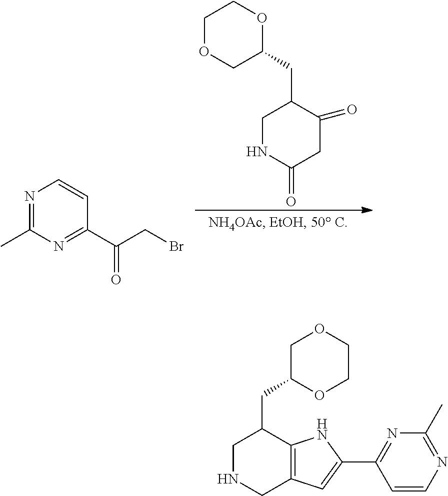 [SMILES: Cc1nccc(C(=O)CBr)n1 ; O=C1CC(=O)C(C[C@@H]2COCCO2)CN1 ; Cc1nccc(-c2cc3c([nH]2)C(C[C@@H]2COCCO2)CNC3)n1]

A mixture of 5-(((R)-1,4-dioxan-2-yl)methyl)piperidine-2,4-dione (14 g, 83 mmol, 1.0 equiv), 2-bromo-1-(2-methylpyrimidin-4-yl)ethan-1-one (18 g, 107 mmol, 1.3 equiv), and NH₄OAc (50.3 g, 830 mmol, 10 equiv) in EtOH (150.0 mL) was stirred at 50 degrees C. for 3 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (10:1)) to afford 7-(((R)-1,4-dioxan-2-yl)methyl)-2-(2-methylpyrimidin-4-yl)-4,5,6,7-tetrahydro-1H-pyrrolo[3,2-c]pyridine (9 g, 57%) as a yellow solid.

LC-MS: (M+H)⁺ found 329.

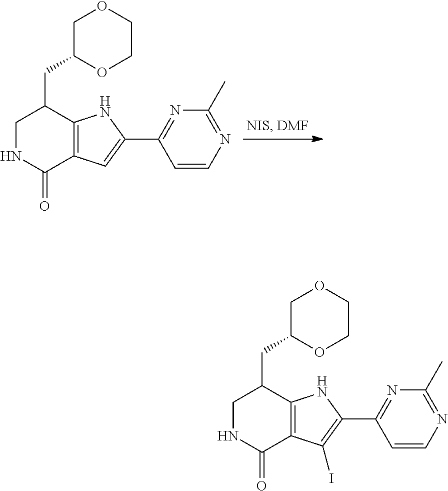 [SMILES: Cc1nccc(-c2cc3c([nH]2)C(C[C@@H]2COCCO2)CNC3=O)n1 ; Cc1nccc(-c2[nH]c3c(c2I)C(=O)NCC3C[C@@H]2COCCO2)n1]

To a stirred mixture of 7-(((R)-1,4-dioxan-2-yl)methyl)-2-(2-methylpyrimidin-4-yl)-1,5,6,7-tetrahydro-4H-pyrrolo[3,2-c]pyridin-4-one (17 g, 51.6 mmol, 1.00 equiv) in DMF (150 mL) was added NIS (15.1 g, 67.8 mmol, 1.3 equiv) at 0 degrees C. The mixture was stirred at room temperature for 2 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (20:1)) to afford 7-(((R)-1,4-dioxan-2-yl)methyl)-3-iodo-2-(2-methylpyrimidin-4-yl)-1,5,6,7-tetrahydro-4H-pyrrolo[3,2-c]pyridin-4-one (16 g, 53%) as yellow solid.

LC-MS: (M+H)⁺ found: 455.

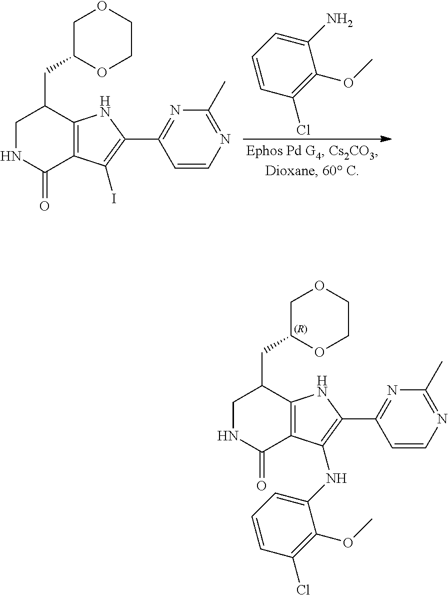

A mixture of 7-[(2R)-1,4-dioxan-2-ylmethyl]-3-iodo-2-(2-methylpyrimidin-4-yl)-1H,5H,6H,7H-pyrrolo[3,2-c]pyridin-4-one (7 g, 15.409 mmol, 1 equiv)Cs₂CO₃ (10.04 g, 30.818 mmol, 2 equiv), 3-chloro-2-methoxyaniline (3.16 g, 20.032 mmol, 1.3 equiv) and EPhos Pd G4 (2.12 g, 2.311 mmol, 0.15 equiv) in DMF was stirred for 3 h at 60° C. under nitrogen atmosphere. The reaction was quenched with Water at room temperature. The resulting mixture was extracted with EtOAc (2×200 mL). The combined organic layers were washed with water (2×200 mL), dried over anhydrous Na2SO4. After filtration, the filtrate was concentrated under reduced pressure. The crude product (5 g) was purified by Prep-HPLC with the following conditions (Column: XBridge Prep OBD C18 Column, 30*150 mm, 5 μm; Mobile Phase A: Water (0.1% FA), Mobile Phase B: MeOH-Preparative; Flow rate: 60 mL/min; Gradient: 30% B to 60% B in 7 min; Wave Length: 254 nm; RT1 (min): 6.5) to afford (7R)-3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (peak2, 1.5814 g, 21.21%) as an orange solid LC-MS: (M+H)⁺ found: 484.

¹H NMR (400 MHz, Chloroform-d) δ 11.41-11.26 (m, 1H), 8.40-8.29 (m, 1H), 7.62-7.55 (m, 1H), 6.85-6.68 (m, 3H), 6.37-6.29 (m, 1H), 5.61-5.52 (m, 1H), 4.25-4.14 (m, 1H), 4.12-4.05 (m, 3H), 4.05-3.73 (m, 5H), 3.50-3.35 (m, 4H), 2.78-2.66 (m, 3H), 1.90-1.77 (m, 1H), 1.63-1.52 (m, 1H).

### Example 112. (7R)-7-[(2R)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (compound 569)

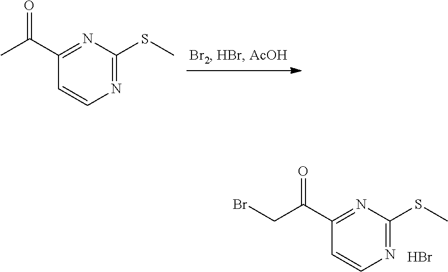

To a stirred mixture of 1-[2-(methylsulfanyl)pyrimidin-4-yl]ethanone (20 g, 118.899 mmol, 1 equiv.) and HBr (20.00 mL, 684.858 mmol, 5.76 equiv.) in AcOH (60 mL) was added Br2 (24.70 g, 154.569 mmol, 1.3 equiv.) at room temperature under nitrogen atmosphere. The resulting mixture was stirred for 3 h at 60° C. Desired product could be detected by LCMS. The precipitated solids were collected by filtration and washed with ethyl acetate (3×20 mL). This resulted in 2-bromo-1-[2-(methylsulfanyl)pyrimidin-4-yl]ethanone hydrobromide (28 g, 71.79%) as a yellow solid.

LC-MS: (M+H)⁺ found: 246.95.

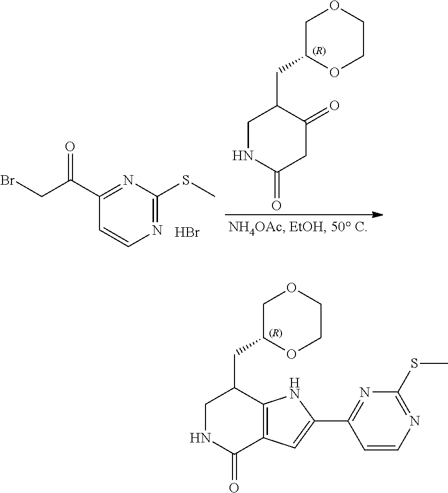

To a stirred solution of 5-[(2R)-1,4-dioxan-2-ylmethyl]piperidine-2,4-dione (3.2 g, 15.007 mmol, 1 equiv.) in EtOH (30 mL) were added 2-bromo-1-[2-(methylsulfanyl)pyrimidin-4-yl]ethanone hydrobromide (5.91 g, 18.008 mmol, 1.2 equiv.) and NH₄OAc (11.57 g, 150.070 mmol, 10 equiv.) at room temperature under nitrogen atmosphere. The resulting mixture was stirred for 2 h at 60° C. Desired product could be detected by LCMS. The aqueous layer was extracted with CH₂Cl₂ (3×100 mL). The resulting mixture was concentrated under reduced pressure. The residue was purified by silica gel column chromatography, eluted with CH₂Cl₂/MeOH (25:1) to afford 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 36.98%) as a brown solid.

LC-MS: (M+H)⁺ found: 361.30.

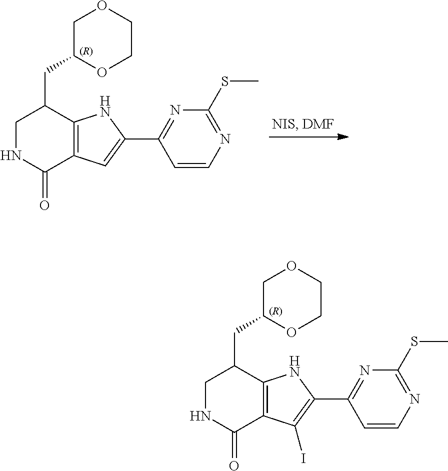

To a stirred solution of 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 5.549 mmol, 1 equiv.) in DMF (20 mL) was added NIS (1.50 g, 6.659 mmol, 1.2 equiv.) at 0° C. under nitrogen atmosphere. The resulting mixture was stirred for overnight at room temperature. Desired product could be detected by LCMS. The aqueous layer was extracted with EtOAc (3×20 mL). The resulting mixture was washed with 2×50 mL of water. The residue was purified by silica gel column chromatography, eluted with CH₂Cl₂/MeOH (30:1) to afford 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.65 g, 61.14%) as a light yellow solid.

LC-MS: (M+H)⁺ found: 487.15.

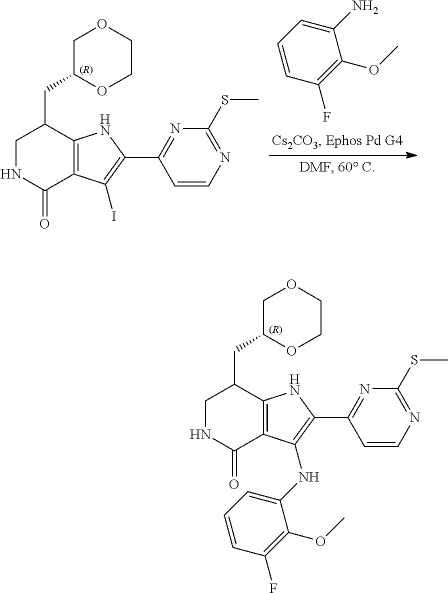

To a stirred mixture of 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.1 g, 2.262 mmol, 1 equiv.) and 3-fluoro-2-methoxyaniline (0.96 g, 6.786 mmol, 3 equiv.) in DMF (15 mL) were added Cs₂CO₃ (2.21 g, 6.786 mmol, 3 equiv.) and Ephos Pd G4 (0.21 g, 0.226 mmol, 0.1 equiv.) at room temperature under nitrogen atmosphere. The resulting mixture was stirred for 1 h at 50° C. Desired product could be detected by LCMS. The residue was purified by reverse flash chromatography with the following conditions: column, silica gel; mobile phase, MeCN in water, 45% to 70% gradient in 10 min; detector, UV 254 nm. This resulted in 7-[(2R)-1,4-dioxan-2-ylmethyl]-3-[(3-fluoro-2-methoxyphenyl)amino]-2-[2-(methylsulfanyl)pyrimidin-4-yl]-1H,5H,6H,7H-pyrrolo[3,2-c]pyridin-4-one (810 mg, 71.69%) as a yellow solid.

LC-MS: (M+H)⁺ found: 500.10

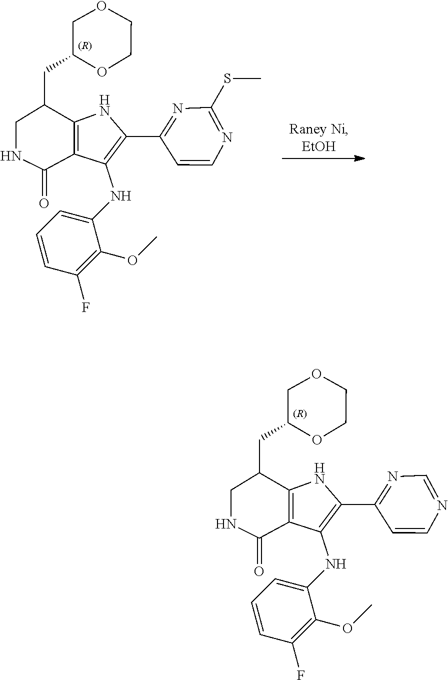

To a stirred solution of (7S)-7-[(2R)-1,4-dioxan-2-ylmethyl]-3-[(3-fluoro-2-methoxyphenyl)amino]-2-[2-(methylsulfanyl)pyrimidin-4-yl]-1H,5H,6H,7H-pyrrolo[3,2-c]pyridin-4-one, 1 equiv.) in EtOH (30 mL) was added Raney-Ni (2.78 g, 32.420 mmol, equiv.) at room temperature under nitrogen atmosphere. The resulting mixture was stirred for 24 h at 90° C. Desired product could be detected by LCMS. The resulting mixture was filtered, the filter cake was washed with EtOH (3×30 mL). The filtrate was concentrated under reduced pressure. This resulted in 7-[(2R)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (600 mg, 81.59%) as a yellow solid.

LC-MS: (M+H)⁺ found: 454.15.

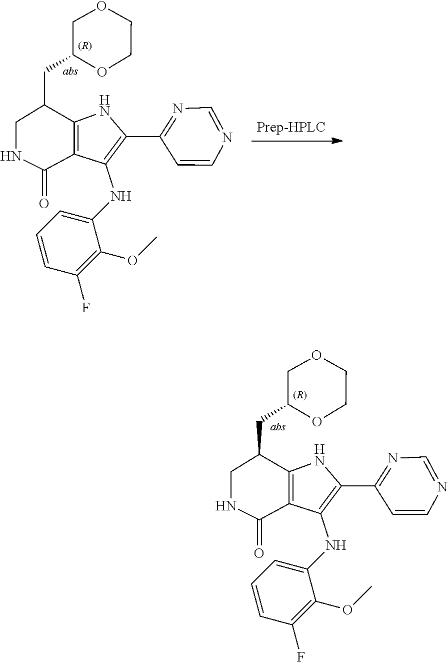

The crude product (600 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 23% B to 37% B in 10 min, 37% B; Wave Length: 254/220 nm; RT1 (min): 7 10) to afford (7R)-7-[(2R)-1,4-dioxan-2-ylmethyl]-3-[(3-fluoro-2-methoxyphenyl)amino]-2-(pyrimidin-4-yl)-1H,5H,6H,7H-pyrrolo[3,2-c]pyridin-4-one (169.8 mg, 26.29%) as a yellow solid.

LC-MS: (M+H)⁺ found: 454.10.

¹H NMR (300 MHz, Chloroform-d) δ 11.29 (s, 1H), 8.72 (d, J=179.0 Hz, 2H), 7.51 (s, 1H), 7.14-6.92 (m, 1H), 6.68 (q, J=7.5 Hz, 1H), 6.61-6.43 (m, 1H), 6.17 (d, J=8.0 Hz, 1H), 5.58 (s, 1H), 4.12 (d, J=12.4 Hz, 4H), 4.01-3.73 (m, 5H), 3.50-3.25 (m, 4H), 1.83-1.77 (m, 1H), 1.54 (d, J=14.7 Hz, 1H).

### Example 113. (7R)-3-[(3-chloro-2-methoxyphenyl)amino]-7-[(2R)-1,4-dioxan-2-ylmethyl]-2-(pyrimidin-4-yl)-1H,5H,6H,7H-pyrrolo[3,2-c]pyridin-4-one (compound 568)

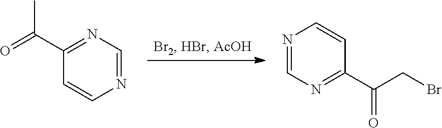

To a stirred solution of 1-(pyrimidin-4-yl)ethanone (1 g, 8.188 mmol, 1.00 equiv) and HBr (1.45 mL, 49.640 mmol, 6.06 equiv) in AcOH (3 mL) was added Br2 (1 g, 6.258 mmol, 0.76 equiv) dropwise at room temperature. Then the mixture was stirred at 60 degree for 3 h. After the reaction was completed, the mixture was diluted with ethyl acetate. The solution was stirred for overnight. The solid was filtrated and dried to get 2-bromo-1-(pyrimidin-4-yl)ethanone (1.3 g, 78.98%) brown solid as product.

LC-MS: (M+H)⁺ found: 200.95.

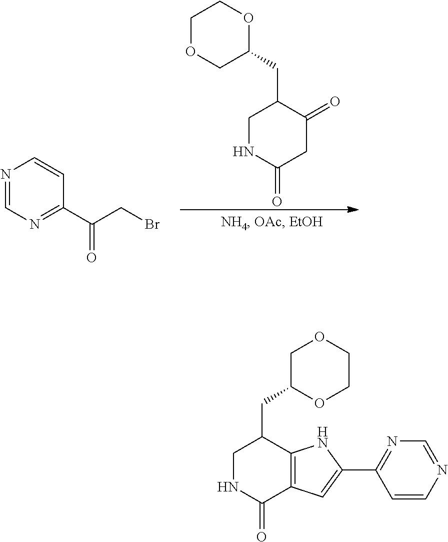 [SMILES: O=C(CBr)c1ccncn1 ; O=C1CC(=O)C(C[C@@H]2COCCO2)CN1 ; O=C1NCC(C[C@@H]2COCCO2)c2[nH]c(-c3ccncn3)cc21]

A solution of 2-bromo-1-(pyrimidin-4-yl)ethanone hydrobromide (3 g, 10.641 mmol, 1 equiv) in EtOH (50 mL) was treated with 5-[(2R)-1,4-dioxan-2-ylmethyl]piperidine-2,4-dione (2.27 g, 10.641 mmol, 1 equiv) for 2 min at room temperature under nitrogen atmosphere followed by the addition of NH₄OAc (8.20 g, 106.410 mmol, 10 equiv) in portions at room temperature. The resulting mixture was stirred for 2 h at 60° C. under nitrogen atmosphere. The resulting mixture was extracted with EtOAc (8×100 mL). The combined organic layers were dried over anhydrous Na2SO4. After filtration, the filtrate was concentrated under reduced pressure. The residue was purified by silica gel column chromatography, eluted with CH2Cl2/MeOH (10:1) to afford 7-[(2R)-1,4-dioxan-2-ylmethyl]-2-(pyrimidin-4-yl)-1H,5H,6H,7H-pyrrolo[3,2-c]pyridin-4-one (1.5 g, 44.84%) as a yellow solid.

LC-MS: (M+H)⁺ found: 315.10.

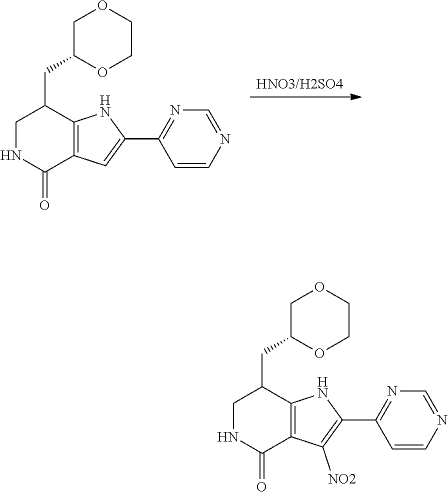 [SMILES: O=C1NCC(C[C@@H]2COCCO2)c2[nH]c(-c3ccncn3)cc21 ; O=C1NCC(C[C@@H]2COCCO2)c2[nH]c(-c3ccncn3)c([N+](=O)[O-])c21]

A solution of 7-[(2R)-1,4-dioxan-2-ylmethyl]-2-(pyrimidin-4-yl)-1H,5H,6H,7H-pyrrolo[3,2-c]pyridin-4-one (700 mg, 2.227 mmol, 1 equiv) in H2S04 (10 mL) was treated with Nitric acid fuming (140.32 mg, 2.227 mmol, 1 equiv) for 2 min at −5° C. under nitrogen atmosphere. The resulting mixture was stirred for 1 h at 0° C. under nitrogen atmosphere. The resulting mixture was diluted with water (40 mL). The mixture was basified to pH 9 with NaOH (aq.). The resulting mixture was extracted with CH₂Cl₂ (5×50 mL). The combined organic layers were dried over anhydrous Na2SO4. After filtration, the filtrate was concentrated under reduced pressure. This resulted in 7-[(2R)-1,4-dioxan-2-ylmethyl]-3-nitro-2-(pyrimidin-4-yl)-1H,5H,6H,7H-pyrrolo[3,2-c]pyridin-4-one (800 mg, 99.97%) as a Brown yellow solid.

LC-MS: (M+H)⁺ found: 360.25.

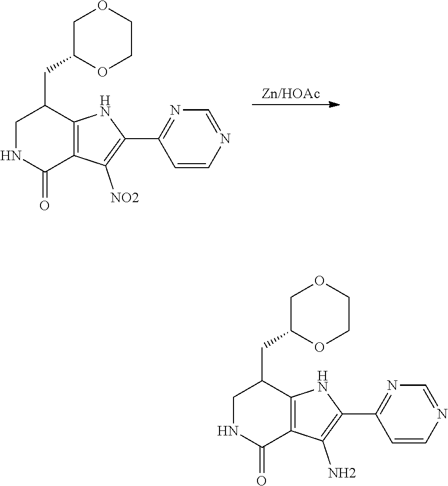 [SMILES: O=C1NCC(C[C@@H]2COCCO2)c2[nH]c(-c3ccncn3)c([N+](=O)[O-])c21 ; Nc1c(-c2ccncn2)[nH]c2c1C(=O)NCC2C[C@@H]1COCCO1]

A solution of 7-[(2R)-1,4-dioxan-2-ylmethyl]-3-nitro-2-(pyrimidin-4-yl)-1H,5H,6H,7H-pyrrolo[3,2-c]pyridin-4-one (800 mg, 2.226 mmol, 1 equiv) in HOAc (10 mL) was treated with Zn (728.10 mg, 11.130 mmol, 5 equiv) for 1 h at room temperature under nitrogen atmosphere. The resulting mixture was diluted with water (30 mL). The mixture was basified to pH 8 with NaOH (aq.). The resulting mixture was extracted with CH2Cl2 (5×300 mL). The combined organic layers were dried over anhydrous Na2SO4. After filtration, the filtrate was concentrated under reduced pressure. This resulted in 3-amino-7-[(2R)-1,4-dioxan-2-ylmethyl]-2-(pyrimidin-4-yl)-1H,5H,6H,7H-pyrrolo[3,2-c]pyridin-4-one (500 mg, 68.19%) as a yellow solid.

LC-MS: (M+H)⁺ found: 330.30.

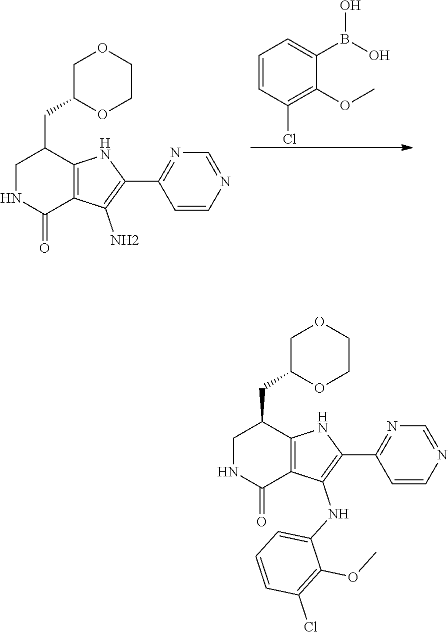 [SMILES: Nc1c(-c2ccncn2)[nH]c2c1C(=O)NCC2C[C@@H]1COCCO1 ; COc1c(Cl)cccc1B(O)O ; COc1c(Cl)cccc1Nc1c(-c2ccncn2)[nH]c2c1C(=O)NC[C@H]2C[C@@H]1COCCO1]

A solution of 3-amino-7-[(2R)-1,4-dioxan-2-ylmethyl]-2-(pyrimidin-4-yl)-1H,5H,6H,7H-pyrrolo[3,2-c]pyridin-4-one (500 mg, 1.518 mmol, 1 equiv) and 3-chloro-2-methoxyphenylboronic acid (565.95 mg, 3.036 mmol, 2 equiv) in DCM (10 mL) was treated with Pyridine (240.16 mg, 3.036 mmol, 2 equiv) and TEA (307.23 mg, 3.036 mmol, 2 equiv) for 2 min at 0° C. under nitrogen atmosphere followed by the addition of Cu(OAc)2 (275.74 mg, 1.518 mmol, 1 equiv) in portions at room temperature. The resulting mixture was stirred for 2 h at room temperature under nitrogen atmosphere. The resulting mixture was extracted with CH₂Cl₂ (3×50 mL). The combined organic layers were washed with brine (2×50 mL), dried over anhydrous Na2SO4. After filtration, the filtrate was concentrated under reduced pressure. The residue was purified by reverse flash chromatography with the following conditions: Column: XBridge Prep OBD C18 Column, 30*150 mm, 5 μm; Mobile Phase A: Water (0.1% FA), Mobile Phase B: ACN; Flow rate: 60 mL/min; Gradient: 20% B to 50% B in 7 min; Wave Length: 254 nm; RT1 (min): 6.5. This resulted in (7R)-3-[(3-chloro-2-methoxyphenyl)amino]-7-[(2R)-1,4-dioxan-2-ylmethyl]-2-(pyrimidin-4-yl)-1H,5H,6H,7H-pyrrolo[3,2-c]pyridin-4-one (8.8 mg) as a yellow solid.

LC-MS: (M+H)⁺ found: 470.35.

¹H NMR (400 MHz, DMSO-d6) δ 12.04 (s, 1H), 9.04 (d, J=1.4 Hz, 1H), 8.56 (d, J=5.6 Hz, 1H), 8.06 (s, 1H), 7.26-7.16 (m, 2H), 6.87-6.76 (m, 2H), 6.34 (dd, J=7.0, 2.7 Hz, 1H), 3.93 (s, 3H), 3.86-3.56 (m, 5H), 3.50 (td, J=10.6, 10.1, 3.6 Hz, 2H), 3.29-3.14 (m, 3H), 1.84 (ddd, J=14.3, 9.2, 5.3 Hz, 1H), 1.65-1.54 (m, 1H).

### Example 114. (7R)-2-(2-aminopyrimidin-4-yl)-3-[(3-chloro-2-methoxyphenyl)amino]-7-[(2R)-1,4-dioxan-2-ylmethyl]-1H,5H,6H,7H-pyrrolo[3,2-c]pyridin-4-one (compound 567)

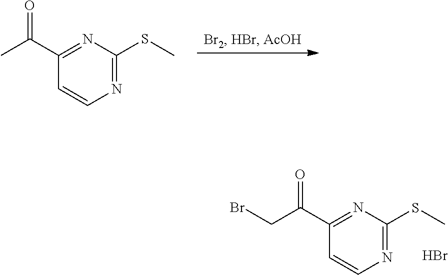

To a stirred mixture of 1-[2-(methylsulfanyl)pyrimidin-4-yl]ethanone (20 g, 118.899 mmol, 1 equiv.) and HBr (20.00 mL, 684.858 mmol, 5.76 equiv.) in AcOH (60 mL) was added Br2 (24.70 g, 154.569 mmol, 1.3 equiv.) at room temperature under nitrogen atmosphere. The resulting mixture was stirred for 3 h at 60° C. Desired product could be detected by LCMS. The precipitated solids were collected by filtration and washed with ethyl acetate (3×20 mL). This resulted in 2-bromo-1-[2-(methylsulfanyl)pyrimidin-4-yl]ethanone hydrobromide (28 g, 71.79%) as a yellow solid.

LC-MS: (M+H)⁺ found: 246.95.

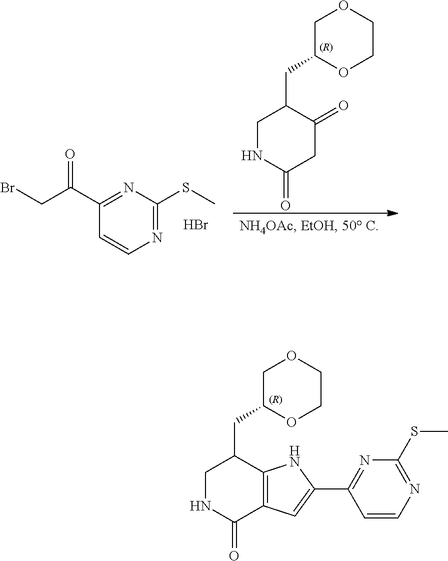

To a stirred solution of 5-[(2R)-1,4-dioxan-2-ylmethyl]piperidine-2,4-dione (3.2 g, 15.007 mmol, 1 equiv.) in EtOH (30 mL) were added 2-bromo-1-[2-(methylsulfanyl)pyrimidin-4-yl]ethanone hydrobromide (5.91 g, 18.008 mmol, 1.2 equiv.) and NH₄OAc (11.57 g, 150.070 mmol, 10 equiv.) at room temperature under nitrogen atmosphere. The resulting mixture was stirred for 2 h at 60° C. Desired product could be detected by LCMS. The aqueous layer was extracted with CH₂Cl₂ (3×100 mL). The resulting mixture was concentrated under reduced pressure. The residue was purified by silica gel column chromatography, eluted with CH₂Cl₂/MeOH (25:1) to afford 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 36.98%) as a brown solid.

LC-MS: (M+H)⁺ found: 361.30.

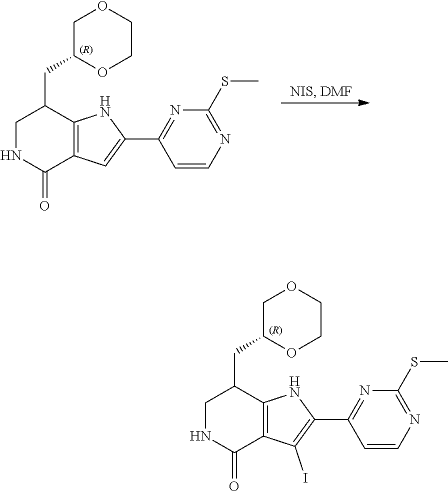

To a stirred solution of 7-[(2R)-1,4-dioxan-2-ylmethyl]-2-[2-(methylsulfanyl)pyrimidin-4-yl]-1H,5H,6H,7H-pyrrolo[3,2-c]pyridin-4-one (2 g, 5.549 mmol, 1 equiv.) in DMF (20 mL) was added NIS (1.50 g, 6.659 mmol, 1.2 equiv.) at 0° C. under nitrogen atmosphere. The resulting mixture was stirred for overnight at room temperature. Desired product could be detected by LCMS. The aqueous layer was extracted with EtOAc (3×20 mL). The resulting mixture was washed with 2×50 mL of water. The residue was purified by silica gel column chromatography, eluted with CH₂Cl₂/MeOH (30:1) to afford 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.65 g, 61.14%) as a light yellow solid.

LC-MS: (M+H)⁺ found: 487.15.

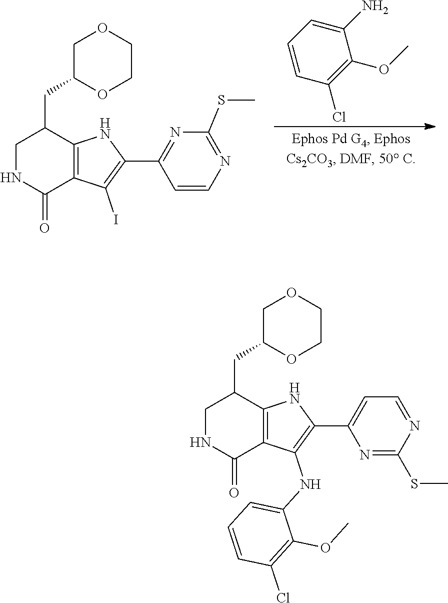

To a stirred mixture of 7-[(2R)-1,4-dioxan-2-ylmethyl]-3-iodo-2-[2-(methylsulfanyl)pyrimidin-4-yl]-1H,5H,6H,7H-pyrrolo[3,2-c]pyridin-4-one (1.5 g, 3.084 mmol, 1 equiv.) and 3-chloro-2-methoxyaniline (1.46 g, 9.252 mmol, 3 equiv.) in DMF (20 mL) were added Cs₂CO₃ (3.01 g, 9.252 mmol, 3 equiv.) and Ephos Pd G4 (0.28 g, 0.308 mmol, 0.1 equiv.) at room temperature under nitrogen atmosphere. The resulting mixture was stirred for 1 h at 50° C. Desired product could be detected by LCMS. The residue was purified by reverse flash chromatography with the following conditions: column, silica gel; mobile phase, MeCN in water, 40% to 60% gradient in 10 min; detector, UV 254 nm. This resulted in 3-[(3-chloro-2-methoxyphenyl)amino]-7-[(2R)-1,4-dioxan-2-ylmethyl]-2-[2-(methylsulfanyl)pyrimidin-4-yl]-1H,5H,6H,7H-pyrrolo[3,2-c]pyridin-4-one (1 g, 62.83%) as a yellow solid.

LC-MS: (M+H)⁺ found: 516.30.

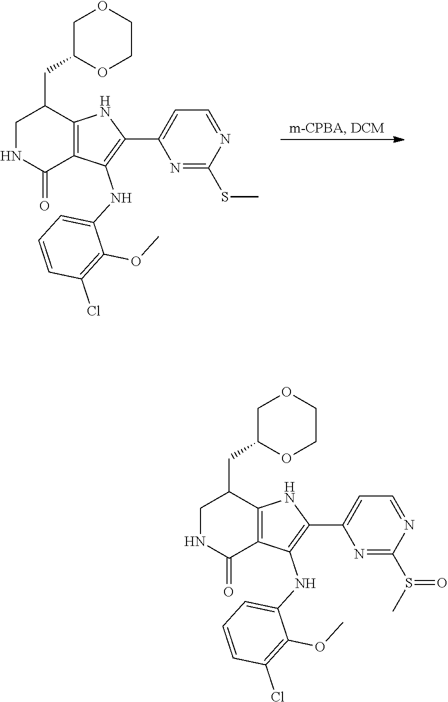 [SMILES: COc1c(Cl)cccc1Nc1c(-c2ccnc(SC)n2)[nH]c2c1C(=O)NCC2C[C@@H]1COCCO1 ; COc1c(Cl)cccc1Nc1c(-c2ccnc(S(C)=O)n2)[nH]c2c1C(=O)NCC2C[C@@H]1COCCO1]

To a stirred solution of 3-[(3-chloro-2-methoxyphenyl)amino]-7-[(2R)-1,4-dioxan-2-ylmethyl]-2-[2-(methylsulfanyl)pyrimidin-4-yl]-1H,5H,6H,7H-pyrrolo[3,2-c]pyridin-4-one (350 mg, 0.678 mmol, 1 equiv.) in DCM (5 mL) was added m-CPBA (234.10 mg, 1.356 mmol, 2 equiv.) at room temperature under nitrogen atmosphere. The resulting mixture was stirred for 10 min at room temperature. Desired product could be detected by LCMS. The aqueous layer was extracted with EtOAc (3×10 mL). The resulting mixture was washed with 3×10 mL of sat.NaHCO₃. The resulting mixture was concentrated under reduced pressure. This resulted in 3-[(3-chloro-2-methoxyphenyl)amino]-7-[(2R)-1,4-dioxan-2-ylmethyl]-2-(2-methanesulfinylpyrimidin-4-yl)-1H,5H,6H,7H-pyrrolo[3,2-c]pyridin-4-one (370 mg, 92.28%) as a reddish solid.

LC-MS: (M+H)⁺ found: 532.45.

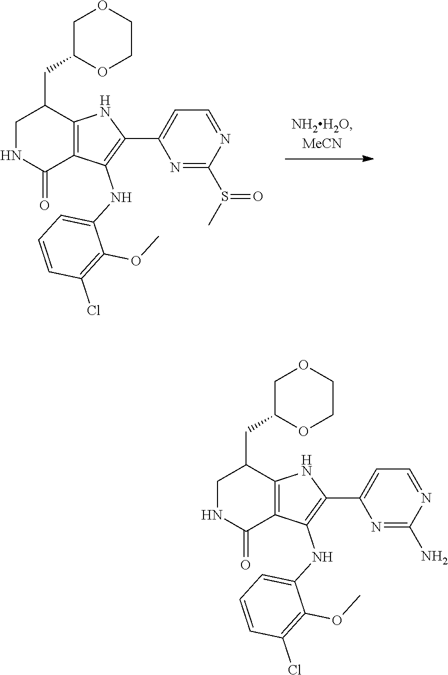 [SMILES: COc1c(Cl)cccc1Nc1c(-c2ccnc(S(C)=O)n2)[nH]c2c1C(=O)NCC2C[C@@H]1COCCO1 ; COc1c(Cl)cccc1Nc1c(-c2ccnc(N)n2)[nH]c2c1C(=O)NCC2C[C@@H]1COCCO1]

To a stirred solution of 3-[(3-chloro-2-methoxyphenyl)amino]-7-[(2R)-1,4-dioxan-2-ylmethyl]-2-(2-methanesulfinylpyrimidin-4-yl)-1H,5H,6H,7H-pyrrolo[3,2-c]pyridin-4-one (370 mg, 0.626 mmol, 1 equiv, 90%) in ACN (4 mL) was added NH3·H2O (2 mL, 15.408 mmol, 24.62 equiv, 30%) at room temperature under nitrogen atmosphere. The resulting mixture was stirred for overnight at 80° C. Desired product could be detected by LCMS. The resulting mixture was concentrated under reduced pressure. This resulted in 150 mg crude product.

LC-MS: (M+H)⁺ found: 485.2.

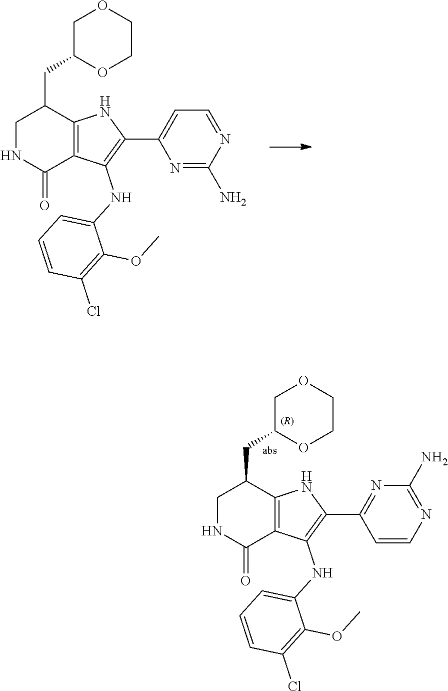

The crude product (150 mg) was purified by Prep-HPLC with the following conditions (Column: XBridge Prep C18 OBD Column, 30*100 mm, 5 μm; Mobile Phase A: Water (10 mmol/L NH4HCO3), Mobile Phase B. ACN; Flow rate: 60 mL/min; Gradient: 25% B to 35% B in 10 min, 35% B; Wave Length: 254/220 nm; RT1 (min): 9.92) to afford (7R)-2-(2-aminopyrimidin-4-yl)-3-[(3-chloro-2-methoxyphenyl)amino]-7-[(2R)-1,4-dioxan-2-ylmethyl]-1H,5H,6H,7H-pyrrolo[3,2-c]pyridin-4-one (27.9 mg, 18.40%) as a yellow solid.

LC-MS: (M+H)⁺ found: 485.2

¹H NMR (300 MHz, Chloroform-d) δ 10.97 (s, 1H), 7.95-7.77 (m, 2H), 6.87-6.68 (m, 2H), 6.40 (t, J=6.3 Hz, 2H), 5.77 (s, 2H), 5.33 (s, 1H), 4.05 (s, 3H), 4.03-3.67 (m, 6H), 3.42 (dt, J=20.4, 8.6 Hz, 4H), 1.57 (d, J=14.7 Hz, 1H), 1.26 (s, 1H).

### Example 115. (7R)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (compound 566)

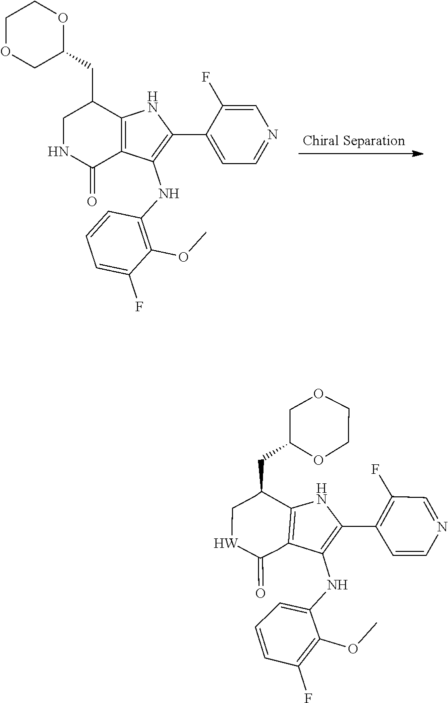

The crude product (100 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 30% B to 60% B in 7 min; Wave Length: 254 nm; RT1 (min): 6.5) to afford (7R)-7-[(2R)-1,4-dioxan-2-ylmethyl]-3-[(3-fluoro-2-methoxyphenyl)amino]-2-(3-fluoropyridin-4-yl)-1H,5H,6H,7H-pyrrolo[3,2-c]pyridin-4-one (257.2 mg) as a white solid.

LCMS: [M+H]⁺ found: 471.

¹H NMR (300 MHz, Chloroform-d) δ 11.06 (s, 1H), 8.44 (d, J=4.7 Hz, 1H), 8.12 (d, J=5.6 Hz, 1H), 7.74 (s, 1H), 7.48-7.37 (m, 1H), 6.69-6.48 (m, 2H), 6.11-6.03 (m, 1H), 5.30 (s, 1H), 4.11 (d, J=1.5 Hz, 4H), 3.95-3.69 (m, 5H), 3.51-3.21 (m, 4H), 1.83-1.72 (m, 1H), 1.52 (d, J=14.8 Hz, 1H).

### Example 116. (R)-7-(((R)-1,4-dioxan-2-yl)methyl)-3-((3-fluoro-2-methoxyphenyl)amino)-2-(2-methylpyrimidin-4-yl)-1,5,6,7-tetrahydro-4H-pyrrolo[3,2-c]pyridin-4-one (compound 605)

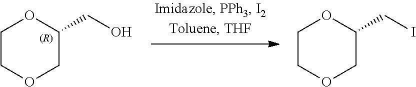

To a suspension of (R)-(1,4-dioxan-2-yl)methanol (100.00 g, 0.847 mol, 1.00 equiv) in Tolune (1000 mL) and THF (500 mL) was added imidazole (115.2 g, 1.69 mol, 2.00 equiv), PPh₃ (214.4 g, 0.847 mol, 1.00 equiv) at room temperature, I₂ (214.4 g, 0.847 mol, 1.00 equiv) was added at 0 degrees C. The mixture was stirred for 3 h at room temperature under nitrogen atmosphere.

Desired product could be detected by LCMS. The resulting solution was quenching with 500 ml of Na₂S₂O₃ saturated solution. The aqueous layer was extracted with EtOAc (3×250 mL), dried (Na₂SO₄) and concentrated. The resulting residue was purified on silica (EtOAc/PE=1:6) to provide 130 g (67%) of the title compound as a colorless oil.

LC-MS: (M+H)⁺ found: 229.

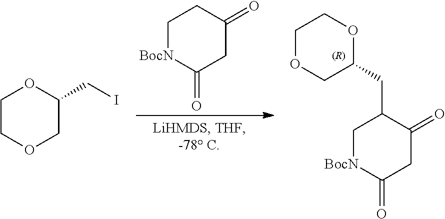

To a suspension of (S)-2-(iodomethyl)-1,4-dioxane (104.00 g, 0.457 mol, 1.40 equiv) and tert-butyl 2,4-dioxopiperidine-1-carboxylate (70.00 g, 0.327 mol, 1.00 equiv) in THF (1000 mL) was dropwise added LiHMDS (1.37 L, 1.37 mol, 3.00 equiv) at −60° C. The mixture was stirred for 1 h at 0 degrees under nitrogen atmosphere.

Desired product could be detected by LCMS. The resulting solution was quenching with 500 ml of HCl (5%) at 0 degrees. The aqueous layer was extracted with EtOAc (3×250 mL), dried (Na₂SO₄) and concentrated. The resulting residue was purified by silica gel column (PE/EtOAc, 1:1) to provide 45 g (46%) of the title compound as a light brown oil.

LC-MS: (M+H)⁺ found: 314.

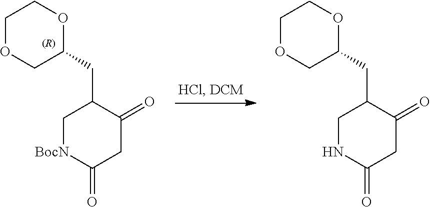

To a stirred mixture of tert-butyl 5-(((R)-1,4-dioxan-2-yl)methyl)-2,4-dioxopiperidine-1-carboxylate (45.00 g) in DCM (300 mL) was added HCl (150 mL) dropwise at 0 degrees C. under nitrogen atmosphere. The mixture was stirred at 0 degrees C. for 1 h. The resulting mixture was concentrated under reduced pressure to afford 30 g crude product, which was used to the next step without further purification.

LC-MS: (M+H)⁺ found: 214.

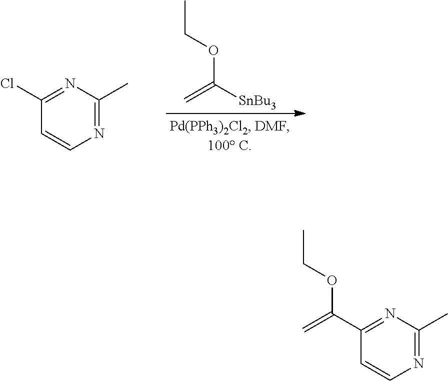

A mixture of 4-chloro-2-methylpyrimidine (20.00 g, 156.2 mmol, 1.00 equiv), tributyl(1-ethoxyvinyl)stannane (45.82 g, 468 mmol, 3.00 equiv) and Pd(PPh₃)₂Cl₂ (8.86 g, 15.6 mmol, 0.10 equiv) in DMF (200.00 mL) was stirred for 12 h at 100 degrees C. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EtOAc (5:1) to afford 4-(1-ethoxyvinyl)-2-methylpyrimidine 17 g (66%) as a yellow oil.

LC-MS: M+H found: 165.0.

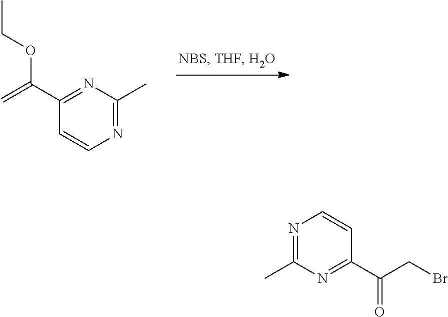

A mixture of 4-(1-ethoxyvinyl)-2-methylpyrimidine (17.00 g, 103.0 mmol, 1.00 equiv), NBS (30.13 g, 133.9 mmol, 1.30 equiv) and H₂O (15 mL) in THF (150 mL) was stirred for 1 h at room temperature. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. This resulted in 2-bromo-1-(2-methylpyrimidin-4-yl) ethan-1-one 18 g crude as a brown oil.

LC-MS: (M+H)⁺ found: 215.

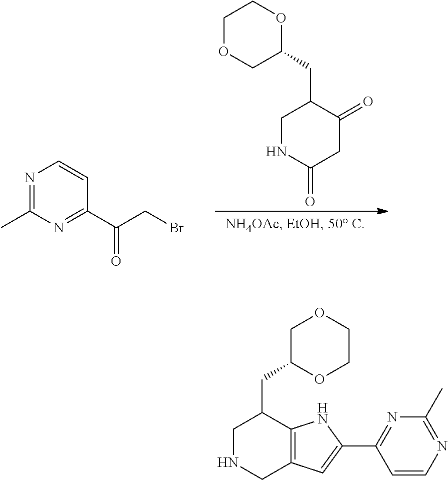 [SMILES: Cc1nccc(C(=O)CBr)n1 ; O=C1CC(=O)C(C[C@@H]2COCCO2)CN1 ; Cc1nccc(-c2cc3c([nH]2)C(C[C@@H]2COCCO2)CNC3)n1]

A mixture of 5-(((R)-1,4-dioxan-2-yl)methyl)piperidine-2,4-dione (14 g, 83 mmol, 1.0 equiv), 2-bromo-1-(2-methylpyrimidin-4-yl)ethan-1-one (18 g, 107 mmol, 1.3 equiv), and NH₄OAc (50.3 g, 830 mmol, 10 equiv) in EtOH (150.0 mL) was stirred at 50 degrees C. for 3 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (10:1)) to afford 7-(((R)-1,4-dioxan-2-yl)methyl)-2-(2-methylpyrimidin-4-yl)-4,5,6,7-tetrahydro-1H-pyrrolo[3,2-c]pyridine (9 g, 57%) as a yellow solid.

LC-MS: (M+H)⁺ found: 329.

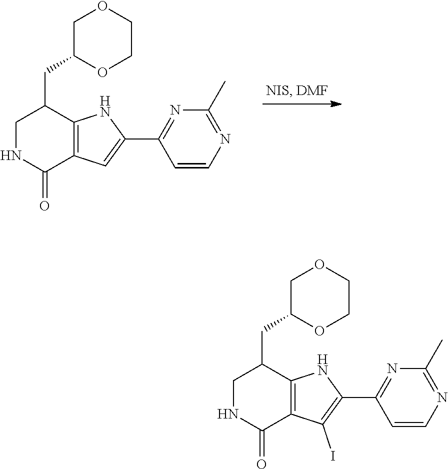 [SMILES: Cc1nccc(-c2cc3c([nH]2)C(C[C@@H]2COCCO2)CNC3=O)n1 ; Cc1nccc(-c2[nH]c3c(c2I)C(=O)NCC3C[C@@H]2COCCO2)n1]

To a stirred mixture of 7-(((R)-1,4-dioxan-2-yl)methyl)-2-(2-methylpyrimidin-4-yl)-1,5,6,7-tetrahydro-4H-pyrrolo[3,2-c]pyridin-4-one (17 g, 51.6 mmol, 1.00 equiv) in DMF (150 mL) was added NIS (15.1 g, 67.8 mmol, 1.3 equiv) at 0 degrees C. The mixture was stirred at room temperature for 2 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (20:1)) to afford 7-(((R)-1,4-dioxan-2-yl)methyl)-3-iodo-2-(2-methylpyrimidin-4-yl)-1,5,6,7-tetrahydro-4H-pyrrolo[3,2-c]pyridin-4-one (16 g, 53%) as yellow solid.

LC-MS: (M+H)⁺ found: 455.

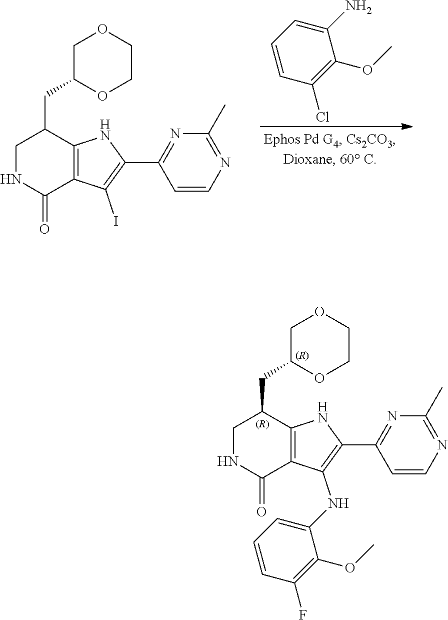

A mixture of 7-[(2R)-1,4-dioxan-2-ylmethyl]-3-iodo-2-(2-methylpyrimidin-4-yl)-1H,5H,6H,7H-pyrrolo[3,2-c]pyridin-4-one (10 g, 22.013 mmol, 1 equiv), Cs₂CO₃ (10.04 g, 30.818 mmol, 2 equiv), 3-fluoro-2-methoxyaniline (4.04 g, 28.617 mmol, 1.3 equiv) and EPhos Pd G4 (2.02 g, 2.201 mmol, 0.1 equiv) in DMF was stirred for overnight at 60° C. under nitrogen atmosphere. The reaction was quenched with Water at room temperature. The resulting mixture was extracted with EtOAc (2×200 mL). The combined organic layers were washed with water (2×200 mL), dried over anhydrous Na₂SO₄. After filtration, the filtrate was concentrated under reduced pressure. The crude product (9 g) was purified by Prep-HPLC with the following conditions (Column: XBridge Shield RP18 OBD Column, 30*150 mm, 5 μm; Mobile Phase A: Water (10 mmol/L NH4HCO3), Mobile Phase B: ACN; Flow rate: 60 mL/min; Gradient: 23% B to 53% B in 8 min, 53% B; Wave Length: 220/254 nm; RT1 (min): 8.02) to afford (R)-7-(((R)-1,4-dioxan-2-yl)methyl)-3-((3-fluoro-2-methoxyphenyl)amino)-2-(2-methylpyrimidin-4-yl)-1,5,6,7-tetrahydro-4H-pyrrolo[3,2-c]pyridin-4-one (1.2586 g, 11.86%) as a yellow solid.

LC-MS: (M+H)⁺ found: 468.

¹H NMR (400 MHz, Chloroform-d) δ 11.42-11.20 (m, 1H), 8.43-8.25 (m, 1H), 7.69-7.56 (m, 1H), 6.93-6.81 (m, 1H), 6.78-6.68 (m, 1H), 6.61-6.54 (m, 1H), 6.26-6.16 (m, 1H), 5.51-5.37 (m, 1H), 4.22-3.78 (m, 9H), 3.50-3.34 (m, 4H), 2.78-2.68 (m, 3H), 1.89-1.78 (m, 1H), 1.63-1.53 (m, 1H).

### Example 117. (7S)-7-[(2R)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (compound 604)

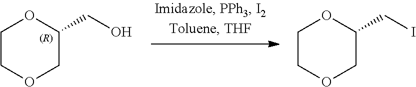

To a suspension of (R)-(1,4-dioxan-2-yl)methanol (100.00 g, 0.847 mol, 1.00 equiv) in Tolune (1000 mL) and THF (500 mL) was added imidazole (115.2 g, 1.69 mol, 2.00 equiv), PPh₃ (214.4 g, 0.847 mol, 1.00 equiv) at room temperature, 12 (214.4 g, 0.847 mol, 1.00 equiv) was added at 0 degrees C. The mixture was stirred for 3 h at room temperature under nitrogen atmosphere.

Desired product could be detected by LCMS. The resulting solution was quenching with 500 ml of Na₂S₂O₃ saturated solution. The aqueous layer was extracted with EtOAc (3×250 mL), dried (Na₂SO₄) and concentrated. The resulting residue was purified on silica (EtOAc/PE=1:6) to provide 130 g (67%) of the title compound as a colorless oil.

LC-MS: (M+H)⁺ found: 229.

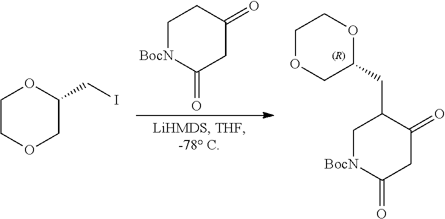

To a suspension of (S)-2-(iodomethyl)-1,4-dioxane (104.00 g, 0.457 mol, 1.40 equiv) and tert-butyl 2,4-dioxopiperidine-1-carboxylate (70.00 g, 0.327 mol, 1.00 equiv) in THF (1000 mL) was dropwise added LiHMDS (1.37 L, 1.37 mol, 3.00 equiv) at −60° C. The mixture was stirred for 1 h at 0 degrees under nitrogen atmosphere.

Desired product could be detected by LCMS. The resulting solution was quenching with 500 ml of HCl (5%) at 0 degrees. The aqueous layer was extracted with EtOAc (3×250 mL), dried (Na₂SO₄) and concentrated. The resulting residue was purified by silica gel column (PE/EtOAc, 1:1) to provide 45 g (46%) of the title compound as a light brown oil.

LC-MS: (M+H)⁺ found; 314.

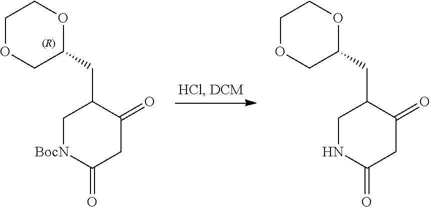

To a stirred mixture of tert-butyl 5-(((R)-1,4-dioxan-2-yl)methyl)-2,4-dioxopiperidine-1-carboxylate (45.00 g) in DCM (300 mL) was added HCl (150 mL) dropwise at 0 degrees C. under nitrogen atmosphere. The mixture was stirred at 0 degrees C. for 1 h. The resulting mixture was concentrated under reduced pressure to afford 30 g crude product, which was used to the next step without further purification.

LC-MS: (M+H)⁺ found: 214.

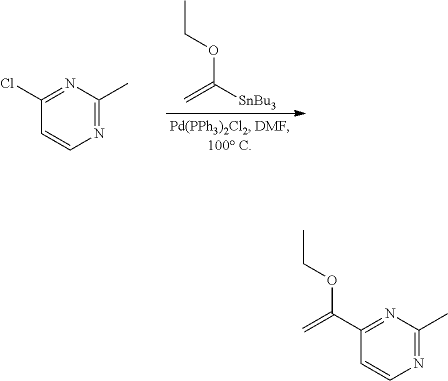

A mixture of 4-chloro-2-methylpyrimidine (20.00 g, 156.2 mmol, 1.00 equiv), tributyl(1-ethoxyvinyl)stannane (45.82 g, 468 mmol, 3.00 equiv) and Pd(PPh₃)₂Cl₂ (8.86 g, 15.6 mmol, 0.10 equiv) in DMF (200.00 mL) was stirred for 12 h at 100 degrees C. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE/EtOAc (5:1) to afford 4-(1-ethoxyvinyl)-2-methylpyrimidine 17 g (66%) as a yellow oil.

LC-MS: M+H found: 165.0.

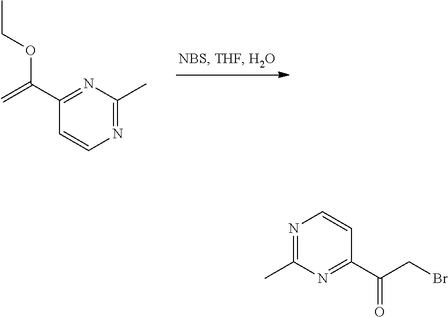

A mixture of 4-(1-ethoxyvinyl)-2-methylpyrimidine (17.00 g, 103.0 mmol, 1.00 equiv), NBS (30.13 g, 133.9 mmol, 1.30 equiv) and H₂O (15 mL) in THF (150 mL) was stirred for 1 h at room temperature. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. This resulted in 2-bromo-1-(2-methylpyrimidin-4-yl) ethan-1-one 18 g crude as a brown oil.

LC-MS: (M+H)⁺ found: 215.

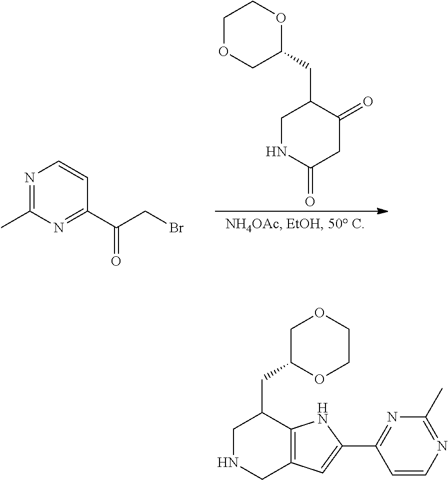 [SMILES: Cc1nccc(C(=O)CBr)n1 ; O=C1CC(=O)C(C[C@@H]2COCCO2)CN1 ; Cc1nccc(-c2cc3c([nH]2)C(C[C@@H]2COCCO2)CNC3)n1]

A mixture of 5-(((R)-1,4-dioxan-2-yl)methyl)piperidine-2,4-dione (14 g, 83 mmol, 1.0 equiv), 2-bromo-1-(2-methylpyrimidin-4-yl)ethan-1-one (18 g, 107 mmol, 1.3 equiv), and NH₄OAc (50.3 g, 830 mmol, 10 equiv) in EtOH (150.0 mL) was stirred at 50 degrees C. for 3 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (10:1)) to afford 7-(((R)-1,4-dioxan-2-yl)methyl)-2-(2-methylpyrimidin-4-yl)-4,5,6,7-tetrahydro-1H-pyrrolo[3,2-c]pyridine (9 g, 57%) as a yellow solid.

LC-MS: (M+H)⁺ found: 329.

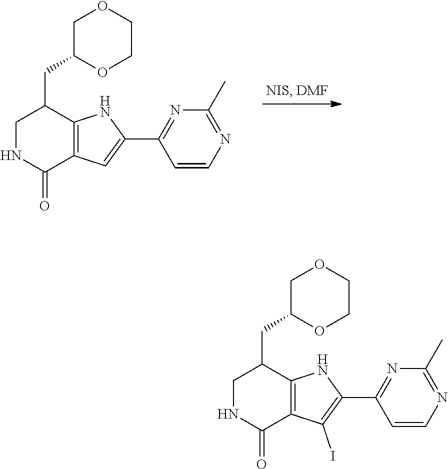 [SMILES: Cc1nccc(-c2cc3c([nH]2)C(C[C@@H]2COCCO2)CNC3=O)n1 ; Cc1nccc(-c2[nH]c3c(c2I)C(=O)NCC3C[C@@H]2COCCO2)n1]

To a stirred mixture of 7-(((R)-1,4-dioxan-2-yl)methyl)-2-(2-methylpyrimidin-4-yl)-1,5,6,7-tetrahydro-4H-pyrrolo[3,2-c]pyridin-4-one (17 g, 51.6 mmol, 1.00 equiv) in DMF (150 mL) was added NIS (15.1 g, 67.8 mmol, 1.3 equiv) at 0 degrees C. The mixture was stirred at room temperature for 2 h. The reaction was quenched with Water at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by Prep-TLC (CH₂Cl₂/MeOH (20:1)) to afford 7-(((R)-1,4-dioxan-2-yl)methyl)-3-iodo-2-(2-methylpyrimidin-4-yl)-1,5,6,7-tetrahydro-4H-pyrrolo[3,2-c]pyridin-4-one (16 g, 53%) as yellow solid.

LC-MS: (M+H)⁺ found: 455.

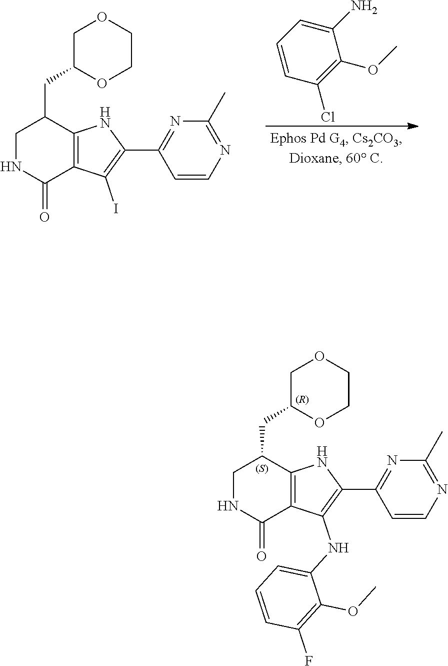

A mixture of 7-[(2R)-1,4-dioxan-2-ylmethyl]-3-iodo-2-(2-methylpyrimidin-4-yl)-1H,5H,6H,7H-pyrrolo[3,2-c]pyridin-4-one (10 g, 22.013 mmol, 1 equiv), Cs₂CO₃ (10.04 g, 30.818 mmol, 2 equiv), 3-fluoro-2-methoxyaniline (4.04 g, 28.617 mmol, 1.3 equiv) and EPhos Pd G4 (2.02 g, 2.201 mmol, 0.1 equiv) in DMF was stirred for overnight at 60° C. under nitrogen atmosphere. The reaction was quenched with Water at room temperature. The resulting mixture was extracted with EtOAc (2×200 mL). The combined organic layers were washed with water (2×200 mL), dried over anhydrous Na₂SO₄. After filtration, the filtrate was concentrated under reduced pressure. The crude product (9 g) was purified by Prep-HPLC with the following conditions (Column: XBridge Shield RP18 OBD Column, 30*150 mm, 5 μm; Mobile Phase A: Water (10 mmol/L NH4HCO3), Mobile Phase B: ACN; Flow rate: 60 mL/min; Gradient: 23% B to 53% B in 8 min, 53% B; Wave Length: 220/254 nm; RT1 (min): 8.02) to afford (7S)-7-[(2R)-1,4-dioxan-2-ylmethyl]-3-[(3-fluoro-2-methoxyphenyl)amino]-2-(2-methylpyrimidin-4-yl)-1H,5H,6H,7H-pyrrolo[3,2-c]pyridin-4-one (1.5675 g, 15.00%) as a yellow solid.

LC-MS: (M+H)⁺ found: 468.

¹H NMR (400 MHz, Chloroform-d) δ 11.42-11.20 (m, 1H), 8.43-8.25 (m, 1H), 7.69-7.56 (m, 1H), 6.93-6.81 (m, 1H), 6.78-6.68 (m, 1H), 6.61-6.54 (m, 1H), 6.26-6.16 (m, 1H), 5.51-5.37 (m, 1H), 4.22-3.78 (m, 9H), 3.50-3.34 (m, 4H), 2.78-2.68 (m, 3H), 1.89-1.78 (m, 1H), 1.63-1.53 (m, 1H).

### Example 118. (7S)-3-[(3-chloro-2-methoxyphenyl)amino]-2-(3-fluoropyridin-4-yl)-7-(2-hydroxy-2-methylpropyl)-1H,5H,6H,7H-pyrrolo[3,2-c]pyridin-4-one (compound 496)

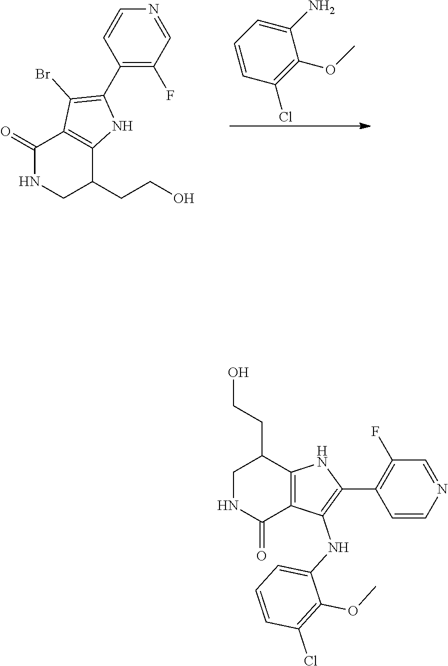

A solution of 3-bromo-2-(3-fluoropyridin-4-yl)-7-(2-hydroxyethyl)-1H,5H,6H,7H-pyrrolo[3,2-c]pyridin-4-one (460 mg, 1.299 mmol, 1.00 equiv) in DMF (10 mL) was treated with 3-chloro-2-methoxyaniline (225.16 mg, 1.429 mmol, 1.1 equiv) and Cs₂CO₃ (1269.50 mg, 3.897 mmol, 3 equiv) for 2 min at room temperature under nitrogen atmosphere followed by the addition of EPhos (104.19 mg, 0.195 mmol, 0.15 equiv) and EPhos Pd G4 (178.95 mg, 0.195 mmol, 0.15 equiv) in portions at room temperature. The resulting mixture was stirred for 2 h at 120 degrees C. under nitrogen atmosphere. The resulting mixture was extracted with CH₂Cl₂ (3×50 mL). The combined organic layers were washed with brine (2×30 mL), dried over anhydrous Na2SO4. After filtration, the filtrate was concentrated under reduced pressure. The residue was purified by reverse flash chromatography with the following conditions: column, silica gel; mobile phase, MeCN in water, 10% to 50% gradient in 10 min; detector, UV 254 nm. This resulted in 3-[(3-chloro-2-methoxyphenyl)amino]-2-(3-fluoropyridin-4-yl)-7-(2-hydroxyethyl)-1H,5H,6H,7H-pyrrolo[3,2-c]pyridin-4-one (170 mg, 30.38%) as a yellow solid.

LC-MS: (M+H)⁺ found: 431.30.

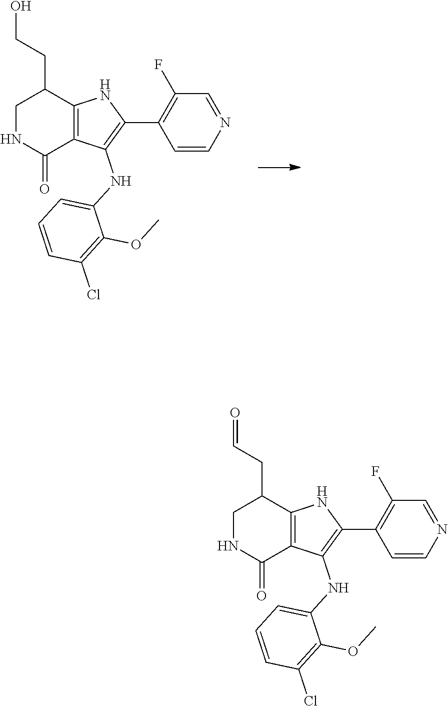 [SMILES: COc1c(Cl)cccc1Nc1c(-c2ccncc2F)[nH]c2c1C(=O)NCC2CCO ; COc1c(Cl)cccc1Nc1c(-c2ccncc2F)[nH]c2c1C(=O)NCC2CC=O]

A solution of 3-[(3-chloro-2-methoxyphenyl)amino]-2-(3-fluoropyridin-4-yl)-7-(2-hydroxyethyl)-1H,5H,6H,7H-pyrrolo[3,2-c]pyridin-4-one (700 mg, 1.625 mmol, 1 equiv) in DCM (10 mL) was treated with Dess-Martin (826.90 mg, 1.950 mmol, 1.2 equiv) for 2 min at 0 degrees C. under nitrogen atmosphere. The resulting mixture was stirred for 2 h at room temperature under nitrogen atmosphere. The resulting mixture was extracted with CH₂Cl₂ (3×50 mL). The combined organic layers were washed with NaHCO₃ (aq.). (2×10 mL), dried over anhydrous Na2SO4. After filtration, the filtrate was concentrated under reduced pressure. The residue was purified by silica gel column chromatography, eluted with CH2Cl2/MeOH (10:1) to afford 2-{3-[(3-chloro-2-methoxyphenyl)amino]-2-(3-fluoropyridin-4-yl)-4-oxo-1H,5H,6H,7H-pyrrolo[3,2-c]pyridin-7-yl}acetaldehyde (400 mg, 57.41%) as a yellow solid.

LC-MS: (M+H)⁺ found: 429.00.

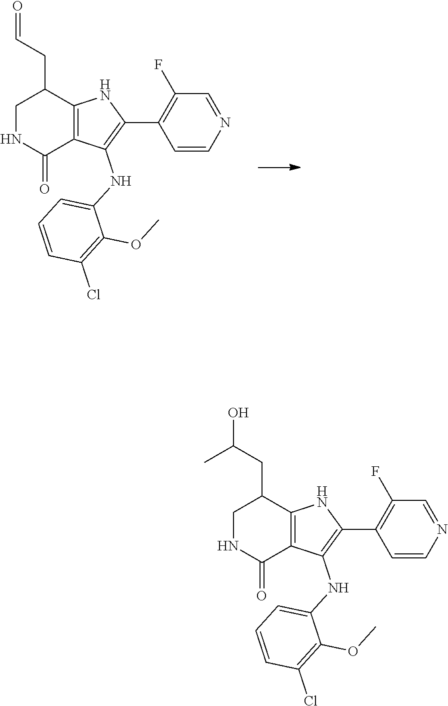 [SMILES: COc1c(Cl)cccc1Nc1c(-c2ccncc2F)[nH]c2c1C(=O)NCC2CC=O ; COc1c(Cl)cccc1Nc1c(-c2ccncc2F)[nH]c2c1C(=O)NCC2CC(C)O]

A solution of 2-{3-[(3-chloro-2-methoxyphenyl)amino]-2-(3-fluoropyridin-4-yl)-4-oxo-1H,5H,6H,7H-pyrrolo[3,2-c]pyridin-7-yl}acetaldehyde (400 mg, 0.933 mmol, 1 equiv) in THF (10 mL) was treated with MeMgBr (222.44 mg, 1.866 mmol, 2 equiv) for 2 h at −5° C. under nitrogen atmosphere. The resulting mixture was diluted with EtOAc (10 mL). The reaction was quenched by the addition of sat. NH4Cl (aq.) (20 mL) at 0° C. The resulting mixture was extracted with EtOAc (3×20 mL). The combined organic layers were dried over anhydrous Na2SO4. After filtration, the filtrate was concentrated under reduced pressure. The residue was purified by silica gel column chromatography, eluted with CH2Cl2/PE (1:1) to afford 3-[(3-chloro-2-methoxyphenyl)amino]-2-(3-fluoropyridin-4-yl)-7-(2-hydroxypropyl)-1H,5H,6H,7H-pyrrolo[3,2-c]pyridin-4-one (170 mg, 40.97%) as a yellow solid.

LC-MS: (M+H)⁺ found: 444.95.

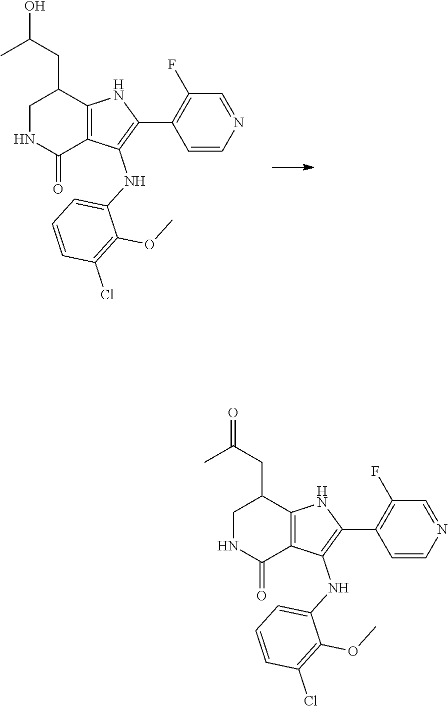 [SMILES: COc1c(Cl)cccc1Nc1c(-c2ccncc2F)[nH]c2c1C(=O)NCC2CC(C)O ; COc1c(Cl)cccc1Nc1c(-c2ccncc2F)[nH]c2c1C(=O)NCC2CC(C)=O]

A solution of 3-[(3-chloro-2-methoxyphenyl)amino]-2-(3-fluoropyridin-4-yl)-7-(2-hydroxypropyl)-1H,5H,6H,7H-pyrrolo[3,2-c]pyridin-4-one (160 mg, 0.360 mmol, 1 equiv) in DCM (4 mL) was treated with Dess-Martin (51.46 mg, 0.432 mmol, 1.2 equiv) for 10 min at 0° C. under nitrogen atmosphere. The resulting mixture was stirred for 1 h at room temperature under nitrogen atmosphere. The resulting mixture was diluted with DCM (10 mL). The reaction was quenched with sat. NaHCO₃ (aq.) at 0° C. The resulting mixture was extracted with CH₂Cl₂ (3×30 mL). The combined organic layers were dried over anhydrous Na2SO4. After filtration, the filtrate was concentrated under reduced pressure. The crude product was used in the next step directly without further purification.

LC-MS: (M+H)⁺ found: 442.95.

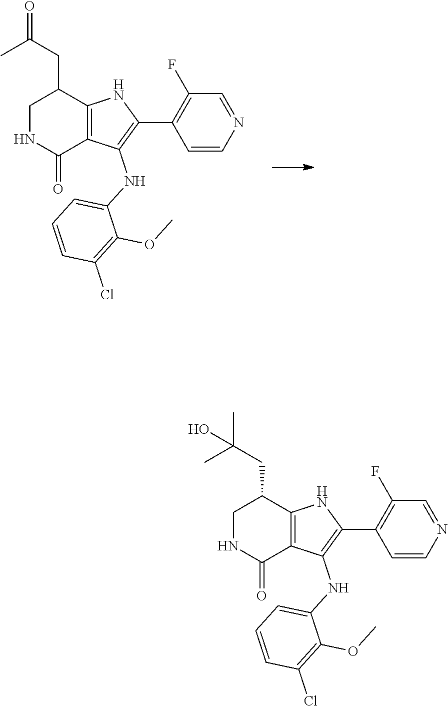 [SMILES: COc1c(Cl)cccc1Nc1c(-c2ccncc2F)[nH]c2c1C(=O)NCC2CC(C)=O ; COc1c(Cl)cccc1Nc1c(-c2ccncc2F)[nH]c2c1C(=O)NC[C@@H]2CC(C)(C)O]

A solution of 3-[(3-chloro-2-methoxyphenyl)amino]-2-(3-fluoropyridin-4-yl)-7-(2-oxopropyl)-1H,5H,6H,7H-pyrrolo[3,2-c]pyridin-4-one (140 mg, 0.316 mmol, 1 equiv) in THF (5 mL) was treated with MeMgBr (75.39 mg, 0.632 mmol, 2 equiv) for 2 h at −5° C. under nitrogen atmosphere. The resulting mixture was diluted with EtOAc (10 mL). The reaction was quenched by the addition of sat. NH4Cl (aq.) (20 mL) at 0° C. The resulting mixture was extracted with EtOAc (3×20 mL). The combined organic layers were dried over anhydrous Na2SO4. After filtration, the filtrate was concentrated under reduced pressure. The crude product (100 mg) was purified by Prep-HPLC with the following conditions ( ) to afford (7S)-3-[(3-chloro-2-methoxyphenyl)amino]-2-(3-fluoropyridin-4-yl)-7-(2-hydroxy-2-methylpropyl)-1H,5H,6H,7H-pyrrolo[3,2-c]pyridin-4-one (30 mg, 19.65%) as a yellow solid.

LC-MS: (M+H)⁺ found: 459.05.

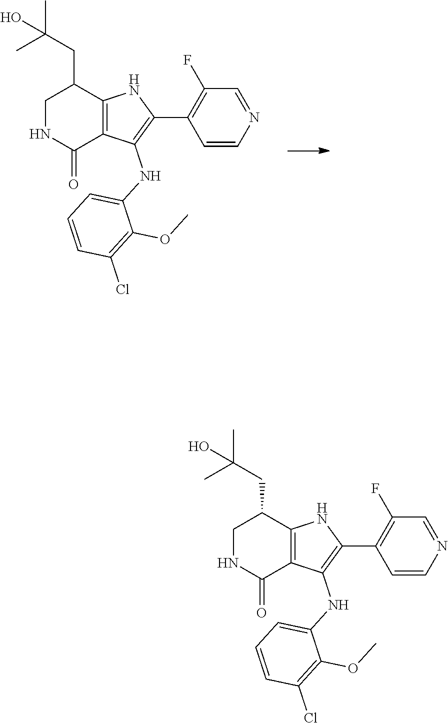 [SMILES: COc1c(Cl)cccc1Nc1c(-c2ccncc2F)[nH]c2c1C(=O)NCC2CC(C)(C)O ; COc1c(Cl)cccc1Nc1c(-c2ccncc2F)[nH]c2c1C(=O)NC[C@@H]2CC(C)(C)O]

The crude product (30 mg) was purified by Prep-HPLC with the following conditions (Column: CHIRALPAK IG-3, 4.6*50 mm, 3 μm; Mobile Phase A: Hex:DCM=3:1)(0.1% DEA): IPA=70: 30; Flow rate: 1 mL/min; Gradient: 0% B to 0% B; Injection Volume: 5 ul mL) to afford (7S)-3-[(3-chloro-2-methoxyphenyl)amino]-2-(3-fluoropyridin-4-yl)-7-(2-hydroxy-2-methylpropyl)-1H,5H,6H,7H-pyrrolo[3,2-c]pyridin-4-one (10.3 mg) as a white solid.

LC-MS: (M+H)⁺ found: 459.00.

¹H NMR (300 MHz, DMSO-d6) δ 11.85 (s, 1H), 8.52 (d, J=3.3 Hz, 1H), 8.25 (d, J=5.1 Hz, 1H), 7.65 (s, 1H), 7.40 (dd, J=7.0, 5.2 Hz, 1H), 7.28 (d, J=3.4 Hz, 1H), 6.77-6.60 (m, 2H), 6.16 (dd, J=6.7, 2.9 Hz, 1H), 5.42 (s, 1H), 3.89 (s, 3H), 3.44-3.34 (m, 1H), 3.24 (d, J=6.3 Hz, 2H), 1.81 (dd, J=14.3, 6.0 Hz, 1H), 1.68-1.56 (m, 1H), 1.26 (d, J=9.7 Hz, 6H).

### Example 119. (7R)-3-[(3-chloro-2-methoxyphenyl)amino]-2-(3-fluoropyridin-4-yl)-7-(2-hydroxy-2-methylpropyl)-1H,5H,6H,7H-pyrrolo[3,2-c]pyridin-4-one (compound 495)

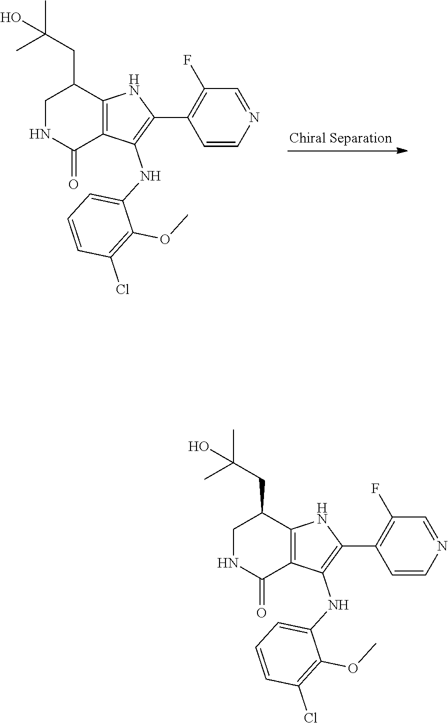 [SMILES: COc1c(Cl)cccc1Nc1c(-c2ccncc2F)[nH]c2c1C(=O)NCC2CC(C)(C)O ; COc1c(Cl)cccc1Nc1c(-c2ccncc2F)[nH]c2c1C(=O)NC[C@H]2CC(C)(C)O]

The crude product (30 mg) was purified by Prep-HPLC with the following conditions (Column: CHIRALPAK IG-3, 4.6*50 mm, 3 μm; Mobile Phase A: Hex:DCM=3:1) (0.1% DEA): IPA=70: 30; Flow rate: 1 mL/min; Gradient: 0% B to 0% B; Injection Volume: 5 ul mL) to afford (7R)-3-[(3-chloro-2-methoxyphenyl)amino]-2-(3-fluoropyridin-4-yl)-7-(2-hydroxy-2-methylpropyl)-1H,5H,6H,7H-pyrrolo[3,2-c]pyridin-4-one (10.7 mg) as a yellow solid.

LC-MS: (M+H)⁺ found: 459.00.

¹H NMR (300 MHz, DMSO-d6) δ 11.90 (s, 1H), 8.61-8.54 (m, 1H), 8.27 (d, J=5.3 Hz, 1H), 7.71 (s, 1H), 7.42 (dd, J=7.0, 5.2 Hz, 1H), 7.34-7.22 (m, 1H), 6.78-6.64 (m, 2H), 6.17 (dd, J=7.2, 2.5 Hz, 1H), 5.42 (s, 1H), 3.89 (s, 3H), 3.39 (s, 1H), 3.24 (d, J=6.6 Hz, 2H), 1.81 (dd, J=14.4, 6.1 Hz, 1H), 1.69-1.57 (m, 1H), 1.26 (d, J=9.7 Hz, 6H).

### Example 120. 3-[(3-chloro-2-methoxyphenyl)amino]-7-[(2R)-1,4-dioxan-2-ylmethyl]-2-(2-methylpyrimidin-4-yl)-1H,5H-pyrrolo[3,2-c]pyridin-4-one (compound 655)

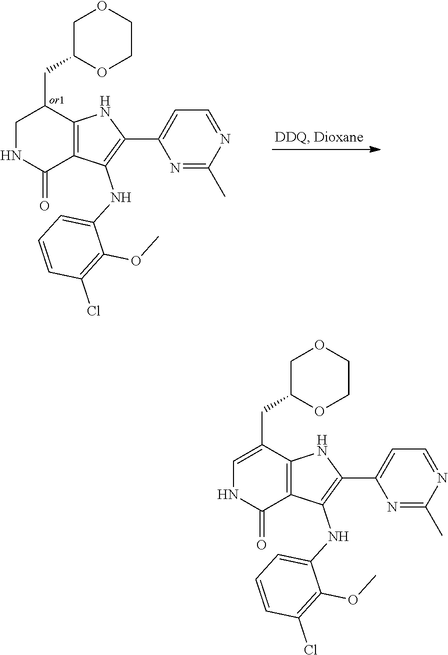 [SMILES: COc1c(Cl)cccc1Nc1c(-c2ccnc(C)n2)[nH]c2c1C(=O)NCC2C[C@@H]1COCCO1 ; COc1c(Cl)cccc1Nc1c(-c2ccnc(C)n2)[nH]c2c(C[C@@H]3COCCO3)c[nH]c(=O)c12]

To a stirred solution of 3-[(3-chloro-2-methoxyphenyl)amino]-7-[(2R)-1,4-dioxan-2-ylmethyl]-2-(2-methylpyrimidin-4-yl)-1H,5H,6H,7H-pyrrolo[3,2-c]pyridin-4-one (150 mg, 0.310 mmol, 1 equiv) in dioxane (3.75 mL, 0.084 mmol, 0.27 equiv) was added DDQ (105.54 mg, 0.465 mmol, 1.5 equiv) in portions at room temperature under nitrogen atmosphere. The resulting mixture was stirred for 1 h at 100° C. under nitrogen atmosphere. The resulting mixture was extracted with EtOAc (2×50 mL). The combined organic layers were washed with brine, dried over anhydrous Na₂SO₄. After filtration, the filtrate was concentrated under reduced pressure. The crude product (100 mg) was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 30% B to 47% B in 10 min, 47% B; Wave Length: 254/220 nm; RT1 (min): 8.17) to afford 3-[(3-chloro-2-methoxyphenyl)amino]-7-[(2R)-1,4-dioxan-2-ylmethyl]-2-(2-methylpyrimidin-4-yl)-1H,5H-pyrrolo[3,2-c]pyridin-4-one (31.8 mg, 20.80%) as a yellow solid.

LCMS: [M+H]⁺ found: 482.

¹H NMR (300 MHz, Chloroform-d) 11.17 (s, 1H), 10.05 (s, 1H), 8.36 (d, J=5.8 Hz, 1H), 7.87 (s, 1H), 7.02-6.83 (m, 3H), 6.84-6.71 (m, 1H), 6.39-6.32 (m, 1H), 4.10 (s, 4H), 3.94-3.73 (m, 5H), 3.49-3.40 (m, 1H), 2.81 (s, 4H), 2.67 (d, J=15.6 Hz, 1H).

### Example 121. 2-(2-aminopyrimidin-4-yl)-3-(1,3-benzothiazol-4-ylamino)-1H,5H,6H,7H-pyrrolo[3,2-c]pyridin-4-one; trifluoroacetic acid (compound 718)

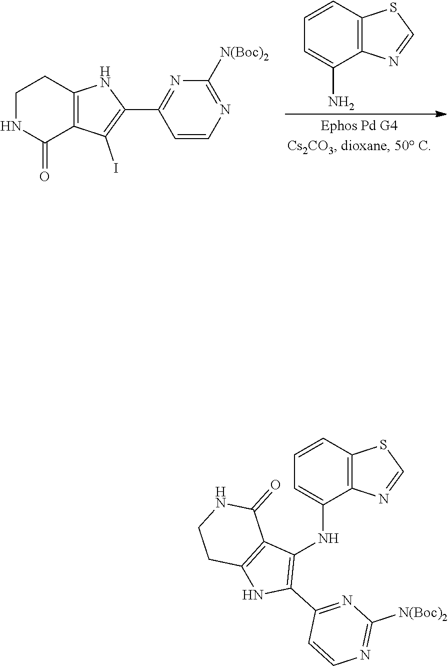

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100.00 mg, 0.18 mmol, 1.00 equiv) in dioxane (2.00 mL) was added Cs₂CO₃ (117.00 mg, 0.36 mmol, 2.00 equiv) and EPhos Pd G4 (33.00 mg, 0.03 mmol, 0.20 equiv) in portions at room temperature under nitrogen atmosphere. To the above mixture was added 1,3-benzothiazol-4-amine (32.00 mg, 0.21 mmol, 1.20 equiv) in portions at room temperature. The resulting mixture was stirred for additional 3 h at 50° C. The reaction was monitored by LCMS. The resulting mixture was concentrated under vacuum. The residue was purified by Prep-TLC (CHCl₃/MeOH 10:1) to afford tert-butyl N-[4-[3-(1,3-benzothiazol-4-ylamino)-4-oxo-1H,5H,6H,7H-pyrrolo[3,2-c]pyridin-2-yl]pyrimidin-2-yl]-N-(tert-butoxycarbonyl)carbamate (90.00 mg, 86.53%) as a yellow solid.

LC-MS: (M+H)⁺ found: 578.3.

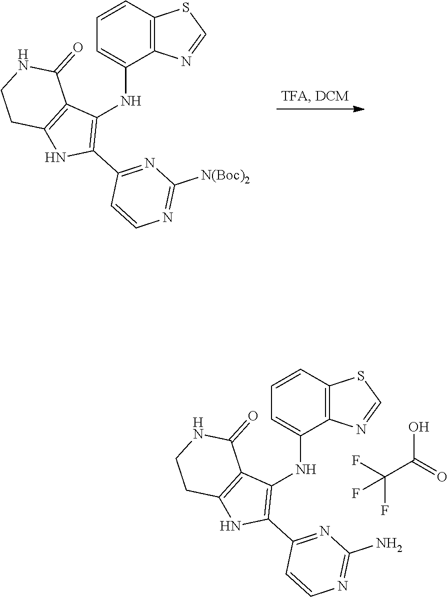

To a stirred solution of tert-butyl N-[4-[3-(1,3-benzothiazol-4-ylamino)-4-oxo-1H,5H,6H,7H-pyrrolo[3,2-c]pyridin-2-yl]pyrimidin-2-yl]-N-(tert-butoxycarbonyl)carbamate (90.00 mg, 0.15 mmol, 1.00 equiv) in DCM (1 mL) was added TFA (1 mL) dropwise at room temperature under nitrogen atmosphere. The resulting mixture was stirred for additional 1 h at room temperature. The reaction was monitored by LCMS. The resulting mixture was concentrated under reduced pressure. The crude product (60.00 mg) was purified by Prep-HPLC with the following conditions (Column: XBridge Shield RP18 OBD Column, 19*250 mm, 10 um; Mobile Phase A: Water (0.05% TFA), Mobile Phase B: MeOH-Preparative; Flow rate: 25 mL/min; Gradient: 37% B to 49% B in 7 min, 49% B; Wave Length: 254 nm; RT1 (min): 5.93) to afford 2-(2-aminopyrimidin-4-yl)-3-(1,3-benzothiazol-4-ylamino)-1H,5H,6H,7H-pyrrolo[3,2-c]pyridin-4-one; trifluoroacetic acid (48.5 mg, 63.34%) as a yellow solid.

LC-MS: (M+H)⁺ found: 378.0.

¹H NMR (400 MHz, DMSO-d6) δ 12.10 (s, 1H), 9.62 (s, 1H), 9.35 (s, 1H), 8.02 (d, J=6.7 Hz, 1H), 7.56-7.36 (m, 4H), 7.23 (t, J=8.0 Hz, 1H), 6.69-6.62 (m, 2H), 3.45-3.41 (m, 2H), 2.92 (t, J=8.0 Hz, 2H).

### Example 122. (S)-((3-chloro-2-methoxyphenyl)amino)-2-(2-((oxetan-2-ylmethyl)amino) pyrimidin-4-yl)-1,5,6,7-tetrahydro-4H-pyrrolo[3,2-c]pyridin-4-one (compound 719)

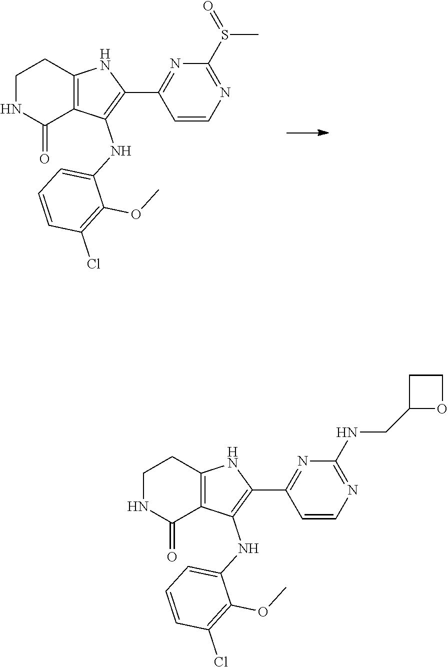 [SMILES: COc1c(Cl)cccc1Nc1c(-c2ccnc(S(C)=O)n2)[nH]c2c1C(=O)NCC2 ; COc1c(Cl)cccc1Nc1c(-c2ccnc(NCC3CCO3)n2)[nH]c2c1C(=O)NCC2]

To a stirred solution of 3-[(3-chloro-2-methoxyphenyl)amino]-2-(2-methanesulfinylpyrimidin-4-yl)-1H,5H,6H,7H-pyrrolo[3,2-c]pyridin-4-one (270.00 mg, 0.63 mmol, 1.00 equiv) in IPA (1.50 mL) and MeCN (1.50 mL) was added 1-(oxetan-2-yl)methanamine (796.00 mg, 9.16 mmol, 15.00 equiv). The resulting mixture was stirred for additional 1 h at 80° C. The reaction was monitored by TLC and LCMS. The precipitated solid was collected by filtration and purified by Prep-HPLC with the following conditions (Column: Column Size: 4.6*50 mm, 3 um, mobile Phase: (Hex:DCM=3:1) (0.1% DEA):IPA=70:30 Flow: 1 mL/min Temperature: 25 degree: Wavelength1: 254 nm) to afford (R)-((3-chloro-2-methoxyphenyl)amino)-2-(2-((oxetan-2-ylmethyl)amino)pyrimidin-4-yl)-1,5,6,7-tetrahydro-4H-pyrrolo[3,2-c]pyridin-4-one (51.70 mg, 18.20%) as a yellow solid.

LC-MS: (M+H)⁺ found: 455.1.

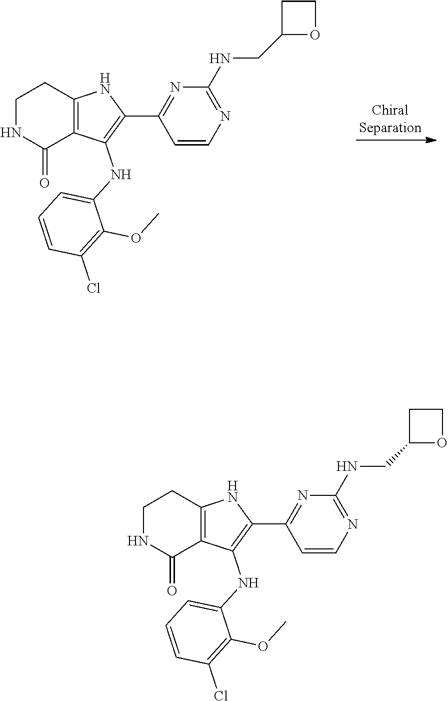 [SMILES: COc1c(Cl)cccc1Nc1c(-c2ccnc(NCC3CCO3)n2)[nH]c2c1C(=O)NCC2 ; COc1c(Cl)cccc1Nc1c(-c2ccnc(NC[C@@H]3CCO3)n2)[nH]c2c1C(=O)NCC2]

The crude product (51.70 mg) was purified by CHLRAL_HPLC with the following conditions (Column: CHIRALPAK ID-3, 4.6*50 mm, 3 um; Mobile Phase A: (Hex:DCM=3:1) (0.1% DEA):IPA=70:30, Flow rate: 1.0/min; 254m; 20 nm) to (S)-((3-chloro-2-methoxyphenyl)amino)-2-(2-((oxetan-2-ylmethyl)amino) pyrimidin-4-yl)-1,5,6,7-tetrahydro-4H-pyrrolo[3,2-c]pyridin-4-one (11.10 mg, 4.10%) as a yellow solid.

LC-MS: (M+H)⁺ found: 455.1.

¹H NMR (300 MHz, DMSO-d₆) δ 11.49 (s, 1H), 7.95 (d, J=5.3 Hz, 1H), 7.79 (s, 1H), 7.01 (s, 1H), 6.78-6.57 (m, 3H), 6.38 (d, J=5.3 Hz, 1H), 6.26 (dd, J=7.6, 2.0 Hz, 1H), 4.70 (m, 1H), 4.34 (m, 2H), 3.76 (s, 3H), 3.60-3.49 (m, 1H), 3.47-3.35 (m, 1H), 3.29 (td, J=6.7, 2.5 Hz, 2H), 2.75 (t, J=6.0 Hz, 2H), 2.52-2.44 (m, J H), 2.29-2.24 (m, 1H).

### Example 123. (R)-((3-chloro-2-methoxyphenyl)amino)-2-(2-((oxetan-2-ylmethyl)amino)pyrimidin-4-yl)-1,5,6,7-tetrahydro-4H-pyrrolo[3,2-c]pyridin-4-one (compound 720)

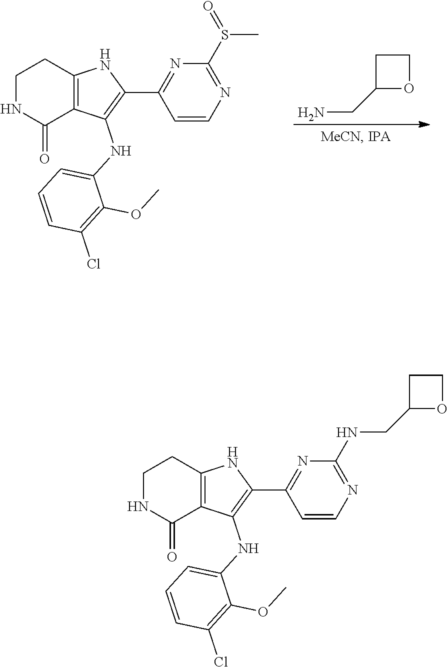 [SMILES: COc1c(Cl)cccc1Nc1c(-c2ccnc(S(C)=O)n2)[nH]c2c1C(=O)NCC2 ; NCC1CCO1 ; COc1c(Cl)cccc1Nc1c(-c2ccnc(NCC3CCO3)n2)[nH]c2c1C(=O)NCC2]

To a stirred solution of 3-[(3-chloro-2-methoxyphenyl) amino]-2-(2-methanesulfinylpyrimidin-4-yl)-1H,5H,6H,7H-pyrrolo[3,2-c]pyridin-4-one (270.00 mg, 0.63 mmol, 1.00 equiv) in IPA (1.50 mL) and MeCN (1.50 mL) was added 1-(oxetan-2-yl)methanamine (796.00 mg, 9.16 mmol, 15.00 equiv). The resulting mixture was stirred for additional 1 h at 80° C. The reaction was monitored by TLC and LCMS. The precipitated solids were collected by filtration and purified by Prep-HPLC with the following conditions (Column: Column Size: 4.6*50 mm, 3 um, mobile Phase: (Hex:DCM=3:1) (0.1% DEA):IPA=70:30 Flow: 1 mL/min Temperature: 25 degree. Wavelength1: 254 nm) to afford (R)-((3-chloro-2-methoxyphenyl)amino)-2-(2-((oxetan-2-ylmethyl)amino)pyrimidin-4-yl)-1,5,6,7-tetrahydro-4H-pyrrolo[3,2-c]pyridin-4-one (51.70 mg, 18.20%) as a yellow solid.

LC-MS: M+H found: 455.1.

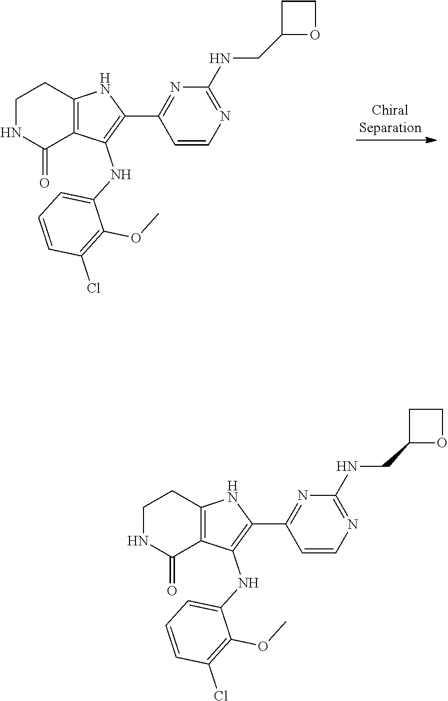 [SMILES: COc1c(Cl)cccc1Nc1c(-c2ccnc(NCC3CCO3)n2)[nH]c2c1C(=O)NCC2 ; COc1c(Cl)cccc1Nc1c(-c2ccnc(NC[C@H]3CCO3)n2)[nH]c2c1C(=O)NCC2]

The crude product (51.70 mg) was purified by CHIRAL_HPLC with the following conditions (Column: CHIRALPAK ID-3, 4.6*50 mm, 3 um; Mobile Phase A: (Hex:DCM=3:1)(0.1% DEA):IPA=70:30, Flow rate: 10.0/min; 254m; 20 nm) to (R)-((3-chloro-2-methoxyphenyl)amino)-2-(2-((oxetan-2-ylmethyl)amino)pyrimidin-4-yl)-1,5,6,7-tetrahydro-4H-pyrrolo[3,2-c]pyridin-4-one (9.40 mg, 3.50%) as a yellow solid.

LC-MS: (M+H)⁺ found: 455.1.

¹H NMR (400 MHz, DMSO-d₆): δ 11.61 (s, 1H), 8.07 (t, J=4.6 Hz, 1H), 7.91 (s, 1H), 7.13 (s, 1H), 6.75-6.85 (m, 3H), 6.50 (t, J=4.7 Hz, 1H), 6.36-6.39 (m, 1H), 4.87-4.76 (m, 1H), 4.40-4.51 (m, 2H), 3.88 (s, 3H), 3.64-3.70 (m, 1H), 3.51-3.56 (m, 1H), 3.07-3.41 (m, 2H), 2.87 (m, 2H), 2.64-2.57 (m, 1H), 2.43-2.34 (m, 1H).

### Example 124. (R)-3-((3-chloro-2-methoxyphenyl)amino)-2-(2-(((tetrahydrofuran-2-yl)methyl)amino)pyrimidin-4-yl)-1,5,6,7-tetrahydro-4H-pyrrolo[3,2-c]pyridin-4-one (compound 721)

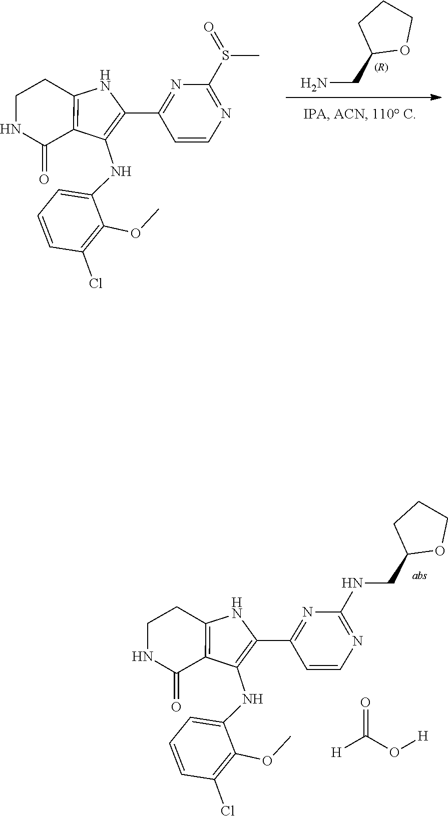

To a stirred mixture of 3-[(3-chloro-2-methoxyphenyl)amino]-2-(2-methanesulfinylpyrimidin-4-yl)-1H,5H,6-H,7H-pyrrolo[3,2-c]pyridin-4-one (80.00 mg, 0.19 mmol, 1.00 equiv), 1-[(2R)-oxolan-2-yl]methanamine (234.00 mg, 2.32 mmol, 10.0 equiv) in MeOH/ACN (1:1, 2 mL) in portions under nitrogen atmosphere. The resulting mixture was stirred for 2 h at 110° C. under nitrogen atmosphere. Desired product could be detected by LCMS. The crude product was purified by Prep-HPLC with the following conditions (Column: Atlantis HILIC OBD Column, 19*150 mm*5 μm; Mobile Phase A: Water (0.1% FA), Mobile Phase B: ACN; Flow rate: 60 mL/min; Gradient: 11% B to 35% B in 7 min, 35% B; Wave Length: 254 nm; RT1 (min): 6.32) to afford (R)-3-((3-chloro-2-methoxyphenyl)amino)-2-(2-(((tetrahydrofuran-2-yl)methyl)amino)pyrimidin-4-yl)-1,5,6,7-tetrahydro-4H-pyrrolo[3,2-c]pyridin-4-one (30.00 mg, 34.00%) as a yellow solid.

LC-MS: (M+H)⁺ found: 469.3.

¹H NMR (300 MHz, DMSO-d₆) δ 11.63 (s, 1H), 8.08 (d, J=5.3 Hz, 1H), 7.96 (s, 1H), 7.15 (s, 1H), 6.87-6.72 (m, 3H), 6.51 (d, J=5.3 Hz, 1H), 6.42-6.35 (m, 1H), 3.96 (t, J=6.3 Hz, 1H), 3.88 (s, 3H), 3.82-3.72 (m, 1H), 3.66-3.58 (m, 1H), 3.45-3.38 (m, 4H), 2.88 (t, J=6.7 Hz, 2H), 1.95-1.73 (m, 3H), 1.45-1.63 (m, 1H).

### Example 125. 3-[(3-chloro-2-methoxyphenyl)amino]-2-(2-[[(2S)-oxolan-2-ylmethyl]amino]pyrimidin-4-yl)-1H,5H,6H,7H-pyrrolo[3,2-c]pyridin-4-one (compound 722)

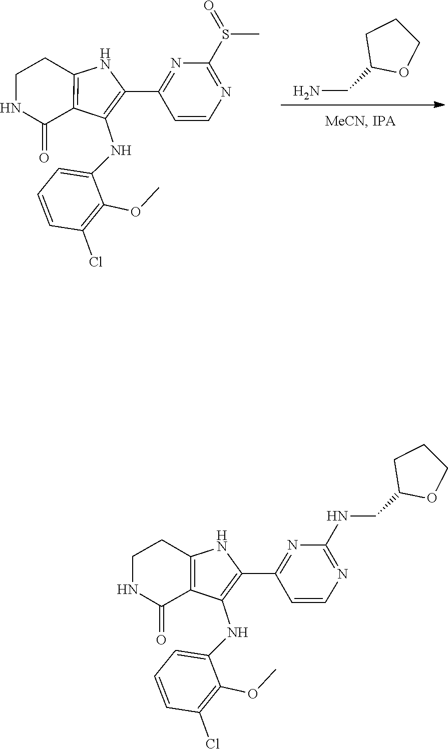

To a stirred solution of 3-[(3-chloro-2-methoxyphenyl)amino]-2-(2-methanesulfinylpyrimidin-4-yl)-1H,5H,6H,7H-pyrrolo[3,2-c]pyridin-4-one (130.00 mg, 0.30 mmol, 1.00 equiv) in IPA (1.00 mL) and MeCN (1.00 mL) was added 1-[(2S)-oxolan-2-yl]methanamine (456.00 mg, 4.52 mmol, 15.00 equiv). The resulting mixture was stirred for overnight at 80° C. The mixture was allowed to cool down to room temperature. The reaction was directly purified by Prep-HPLC with the following conditions ((2 #SHIMADZU (HPLC-01)): Column, Xselect CSH OBD Column 30*150 mm Sum, 254 nm; mobile phase, Water (0.1% FA) and ACN (11% ACN up to 35% in 8 min) to afford 3-[(3-chloro-2-methoxyphenyl)amino]-2-(2-[[(2S)-oxolan-2-ylmethyl]amino]pyrimidin-4-yl)-1H,5H,6H,7H-pyrrolo[3,2-c]pyridin-4-one (32.80 mg, 23.00%) as a yellow solid.

LC-MS: (M+H)⁺ found: 469.1.

¹H NMR (400 MHz, DMSO-d₆) δ 11.63 (s, 1H), 8.19 (s, 1H), 8.14-7.87 (m, 2H), 7.16 (t, J=2.6 Hz, 1H), 7.05-6.61 (m, 3H), 6.51 (d, J=5.3 Hz, 1H), 6.38 (dd, J=7.9, 1.8 Hz, 1H), 4.01-3.94 (m, 1H), 3.88 (s, 3H), 3.81-3.73 (m, 1H), 3.67-3.58 (m, 1H), 3.49-3.30 (m, 4H), 2.87 (t, J=6.7 Hz, 2H), 1.96-1.71 (m, 3H), 1.63-1.50 (m, 1H).

### Example 126. 3-[(3-chloro-2-methoxyphenyl)amino]-2-(2-[[(2S)-1,4-dioxan-2-ylmethyl]amino]pyrimidin-4-yl)-1H,5H,6H,7H-pyrrolo[3,2-c]pyridin-4-one (compound 723)

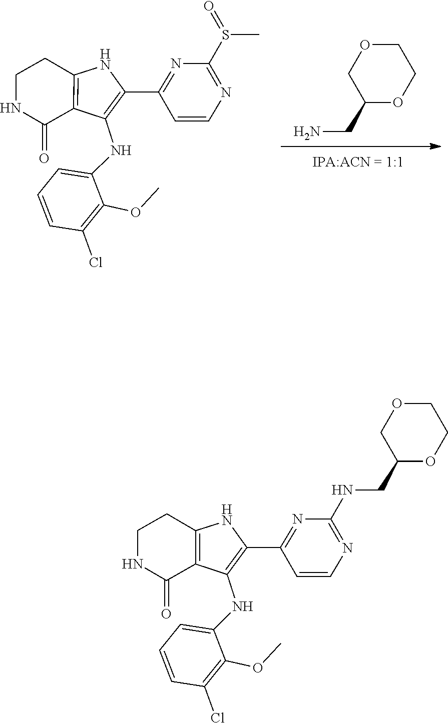

Into an 8 mL pressure tank reactor were added 3-[(3-chloro-2-methoxyphenyl) amino]-2-(2-methanesulfinylpyrimidin-4-yl)-1H,5H,6H,7H-pyrrolo [3,2-c] pyridin-4-one (100.00 mg, 0.23 mmol, 1.00 equiv), 1-[(2S)-1,4-dioxan-2-yl] methanamine (406.86 mg, 3.47 mmol, 15.00 equiv) and IPA (1 mL), ACN (1 mL) at room temperature under air atmosphere. The resulting mixture was stirred for 30 h at 110° C. under air atmosphere.

Desired product could be detected by LCMS. The resulting mixture was concentrated under vacuum. The crude product (100.00 mg) was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 10% B to 32% B in 8 min, 32% B; Wave Length: 254/220 nm; RT1 (min): 7.72) to afford 3-[(3-chloro-2-methoxyphenyl)amino]-2-(2-[[(2S)-1,4-dioxan-2-ylmethyl]amino]pyrimidin-4-yl)-1H,5H,6H,7H-pyrrolo[3,2-c]pyridin-4-one (15.70 mg, 13.98%) as a yellow solid.

LC-MS: (M+H)⁺ found: 485.3.

¹H NMR (300 MHz, DMSO-d₆) δ 11.61 (s, 1H), 8.19-8.04 (m, 1H), 7.89 (s, 1H), 7.16 (d, J=2.7 Hz, 1H), 6.88-6.72 (m, 2H), 6.50 (d, J=5.3 Hz, 1H), 6.37 (dd, J=7.6, 2.0 Hz, 1H), 3.89 (s, 3H), 3.79-3.55 (m, 4H), 3.54-3.35 (m, 5H), 3.26 (dd, J=11.4, 9.7 Hz, 2H), 2.88 (t, J=6.8 Hz, 2H).

### Example 127. 3-[(3-chloro-2-methoxyphenyl)amino]-2-[2-[(1,4-dioxan-2-ylmethyl)amino]pyrimidin-4-yl]-1H,5H,6H,7H-pyrrolo[3,2-c]pyridin-4-one (compound 724)

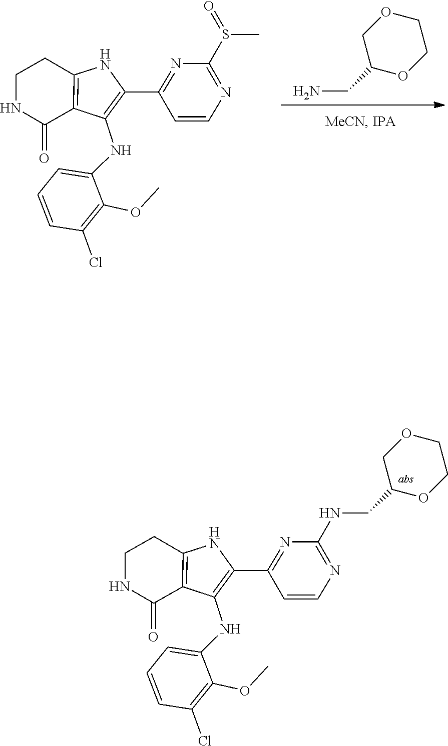

To a stirred solution of 3-[(3-chloro-2-methoxyphenyl) amino]-2-(2-methanesulfinylpyrimidin-4-yl)-1H,5H,6H,7H-pyrrolo[3,2-c] pyridin-4-one (100.00 mg, 0.23 mmol, 1.00 equiv) in IPA (1.00 mL) and MeCN (1.00 mL) was added 1-[(2R)-1,4-dioxan-2-yl] methanamine (271.00 mg, 2.31 mmol, 10.00 equiv) in portions. The resulting mixture was stirred for overnight at 80° C. The mixture was allowed to cool down to room temperature. The reaction was directly purified Chiral-Prep-HPLC with the following conditions (2 #SHIMADZU (HPLC-01)): Column, Xselect CSH F-Phenyl OBD column, 19*250 mm, 5 μm; mobile phase, Water (0.1% FA) and ACN (13% ACN up to 29% in 7 min) to afford 3-[(3-chloro-2-methoxyphenyl)amino]-2-[2-[(1,4-dioxan-2-ylmethyl)amino]pyrimidin-4-yl]-1H,5H,6H,7H-pyrrolo[3,2-c]pyridin-4-one (11.80 mg, 9.60%) as a yellow solid.

LC-MS: (M+H)⁺ found: 485.0.

¹H NMR (300 MHz, DMSO-d6) δ 11.59 (s, 1H), 8.05 (d, J=5.3 Hz, 1H), 7.87 (s, 1H), 7.13 (t, J=2.5 Hz, 1H), 6.85-6.71 (m, 3H), 6.48 (d, J=5.3 Hz, 1H), 6.34 (m, 1H), 3.86 (s, 3H), 3.76-3.67 (m, 2H), 3.66-3.56 (m, 2H), 3.55-3.45 (m, 2H), 3.44-3.41 (m, 1H), 3.38-3.30 (m, 2H), 3.28-3.17 (m, 2H), 2.86 (t, J=6.7 Hz, 2H).

### Example 128. 2-(2-aminopyrimidin-4-yl)-3-[(3-chloro-2-methylphenyl)amino]-1H,5H,6H,7H-pyrrolo[3,2-c]pyridin-4-one; trifluoroacetic acid (compound 725)

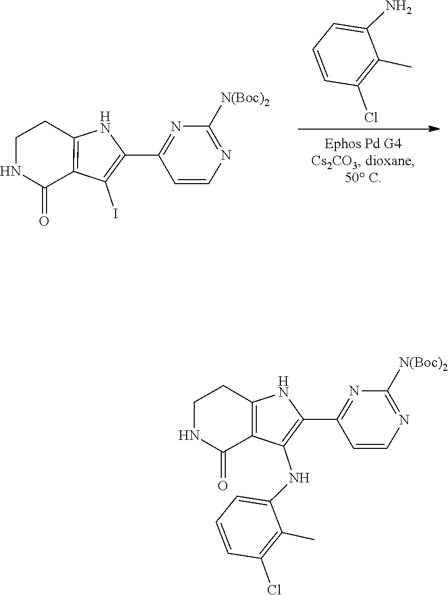

To a stirred mixture of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100.00 mg, 0.18 mmol, 1.00 equiv) and 2-methyl-3-chloroaniline (25.00 mg, 0.18 mmol, 1.00 equiv) in dioxane (2.00 mL) was added EPhos Pd G4 (33.00 mg, 0.03 mmol, 0.20 equiv) and Cs₂CO₃ (117.00 mg, 0.36 mmol, 2.00 equiv) at room temperature under nitrogen atmosphere. The resulting mixture was stirred for 2 h at 50° C. under argon atmosphere.

Desired product could be detected by LCMS. The resulting mixture was concentrated under reduced pressure. The residue was purified by silica gel column chromatography, eluted with CH₂Cl₂/MeOH (10:1) to afford tert-butyl N-(tert-butoxycarbonyl)-N-(4-[3-[(3-chloro-2-methylphenyl)amino]-4-oxo-1H,5H,6H,7H-pyrrolo[3,2-c]pyridin-2-yl]pyrimidin-2-yl)carbamate (50.00 mg, 48.80%) as a yellow solid.

LC-MS: (M+H)⁺ found: 569.1.

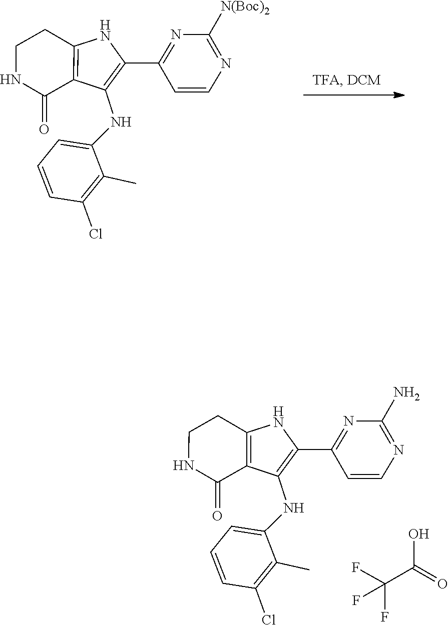

To a stirred solution of tert-butyl N-(tert-butoxycarbonyl)-N-(4-[3-[(3-chloro-2-methylphenyl)amino]-4-oxo-1H,5H,6H,7H-pyrrolo[3,2-c]pyridin-2-yl]pyrimidin-2-yl)carbamate (50.00 mg, 0.08 mmol, 1.00 equiv) in DCM (1.00 mL) was added TFA (1.00 mL) dropwise at 0° C. under nitrogen atmosphere. The resulting mixture was stirred for additional 2 h at 50° C. The reaction was monitored by LCMS. LCMS showed the reaction was completed. The resulting mixture was concentrated under reduced pressure. The crude product (30.00 mg) was purified by Prep-HPLC with the following conditions (Column: Xselect CSH OBD Column 30*150 mm 5 um, n; Mobile Phase A: Water (0.05% TFA), Mobile Phase B: ACN; Flow rate: 60 mL/min; Gradient: 10% B to 35% B in 8 min, 35% B; Wave Length: 254/220 nm; RT1 (min): 7.63) to afford 2-(2-aminopyrimidin-4-yl)-3-[(3-chloro-2-methylphenyl)amino]-1H,5H,6H,7H-pyrrolo[3,2-c]pyridin-4-one; trifluoroacetic acid (13.20 mg, 31.11%) as a yellow solid.

LC-MS: (M+H)⁺ found: 369.1.

¹H NMR (400 MHz, DMSO-d₆) δ 11.97 (s, 1H), 8.90 (s, 1H), 8.00 (d, J=8.0 Hz, 1H), 7.67 (s, 2H), 7.11 (s, 1H), 7.00-6.98 (m, 2H), 6.77 (s, 2H), 3.38-3.35 (m, 2H), 2.85 (t, J=8.0 Hz, 2H), 2.36 (s, 3H).

### Example 129. 3-[(3-chloro-2-methoxyphenyl)amino]-2-(pyrimidin-4-yl)-1H,5H,6H,7H-pyrrolo[3,2-c]pyridin-4-one (compound 675)

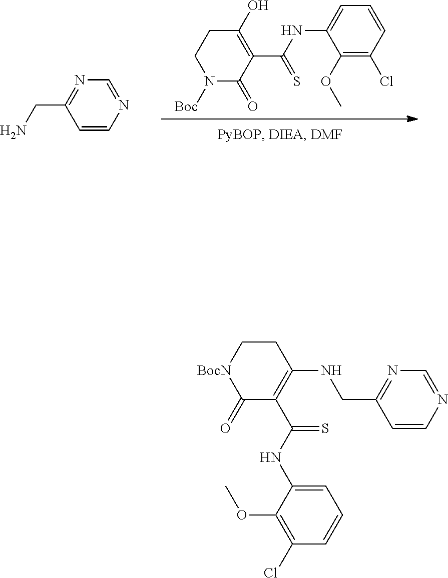 [SMILES: NCc1ccncn1 ; COc1c(Cl)cccc1NC(=S)C1=C(O)CCN(C(=O)OC(C)(C)C)C1=O ; COc1c(Cl)cccc1NC(=S)C1=C(NCc2ccncn2)CCN(C(=O)OC(C)(C)C)C1=O]

To a stirred mixture of 1-(pyrimidin-4-yl)methanamine (1.20 g, 11.00 mmol, 1.00 equiv) and tert-butyl 3-[(3-chloro-2-methoxyphenyl)carbamothioyl]-4-hydroxy-2-oxo-5,6-dihydropyridine-1-carboxylate (4.54 g, 11.00 mmol, 1.00 equiv) and PyBOP (8.58 g, 16.49 mmol, 1.50 equiv) and DIEA (4.26 g, 33.00 mmol, 3.00 equiv) in DMF (40 mL) in portions at room temperature under nitrogen atmosphere. The resulting mixture was stirred for overnight at room temperature under nitrogen atmosphere. Desired product could be detected by LCMS. The residue was purified by reverse flash chromatography with the following conditions (column, C18 silica gel; mobile phase, ACN in water, 10% to 50% gradient in 10 min; detector, UV 254 nm) to afford tert-butyl 3-[(3-chloro-2-methoxyphenyl)carbamothioyl]-2-oxo-4-[(pyrimidin-4-ylmethyl)amino]-5,6-dihydropyridine-1-carboxylate (830.00 mg, 14.98%) as a red oil.

LC-MS: (M+H)⁺ found: 504.0.

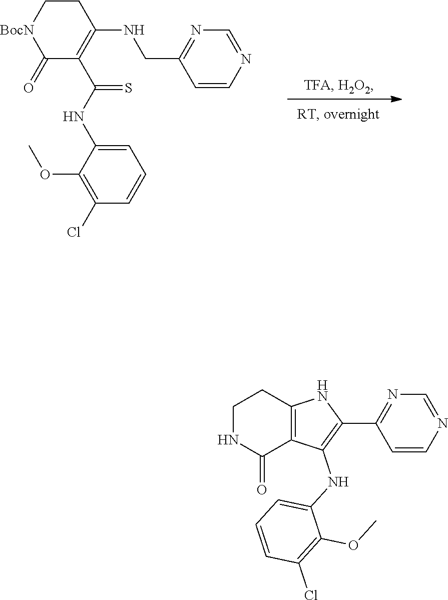

To a stirred mixture of tert-butyl 3-[(3-chloro-2-methoxyphenyl)carbamothioyl]-2-oxo-4-[(pyrimidin-4-ylmethyl)amino]-5,6-dihydropyridine-1-carboxylate (410.00 mg, 0.81 mmol, 1.00 equiv) and TFA (139.10 mg, 1.22 mmol, 1.50 equiv) and H₂O₂ (55.30 mg, 1.63 mmol, 2.00 equiv) in MeOH (8 mL) in portions at room temperature under nitrogen atmosphere. The resulting mixture was stirred for overnight at room temperature under nitrogen atmosphere. The resulting mixture was stirred for 2 h at 80 V under nitrogen atmosphere. Desired product could be detected by LCMS. The crude product (70.00 mg) was purified by Prep-HPLC with the following conditions (Column: XBridge Shield RP18 OBD Column, 30*150 mm, Sum; Mobile Phase A: Water (10 mmol/L NH4HCO3), Mobile Phase B: ACN; Flow rate: 60 mL/min; Gradient. 18% B to 40% B in 10 min, 40% B; Wave Length: 220/254 nm; RT1 (min): 10.72) to afford 3-[(3-chloro-2-methoxyphenyl)amino]-2-(pyrimidin-4-yl)-1H,5H,6H,7H-pyrrolo[3,2-c]pyridin-4-one (31.60 mg, 10.28%) as a light yellow solid.

LC-MS: (M+H)⁺ found: 370.0.

¹H NMR (400 MHz, DMSO-d₆) δ 12.13 (s, 1H), 9.04 (d, J=1.4 Hz, 1H), 8.55 (d, J=5.6 Hz, 1H), 7.93 (s, 1H), 7.34-7.11 (m, 2H), 6.94-6.74 (m, 2H), 6.33 (dd, J=7.0, 2.6 Hz, 1H), 3.93 (s, 3H), 3.41 (td, J=6.8, 2.6 Hz, 2H), 2.87 (t, J=6.8 Hz, 2H).

### Example 130. 3-[(3-acetyl-2-methoxyphenyl)amino]-2-(2-aminopyrimidin-4-yl)-1H,5H,6H,7H-pyrrolo[3,2-c]pyridin-4-one (compound 728)

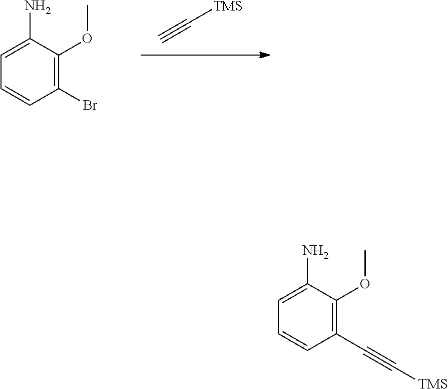

A solution of 3-bromo-2-methoxyaniline (1.00 g, 4.95 mmol, 1.00 equiv), Pd(PPh₃)₂Cl₂ (347.39 mg, 0.50 mmol, 0.10 equiv) and CuI (377.03 mg, 1.98 mmol, 0.40 equiv) was bubbled under N₂ for over 10 minutes. Then trimethylsilylacetylene (2430.57 mg, 24.75 mmol, 5.00 equiv) and DIPA (1.54 mL) was added, and the reaction was stirred at 105° C. overnight. After the reaction was completed, the resulting mixture was concentrated under reduced pressure. The mixture was diluted with EA (10 mL) and washed with NH₄OH/NH₄Cl (1:1, 10 ml), NH₄Cl (10 ml), and brine (20 ml). The organic phase was concentrated. The residue was purified by silica gel column chromatography, eluted with hexane/EtOAc (20:1) to afford 2-methoxy-3-[2-(trimethylsilyl)ethynyl]aniline (930.00 mg, 85.66%) as a brown oil.

LC-MS: (M+H)⁺ found: 220.2.

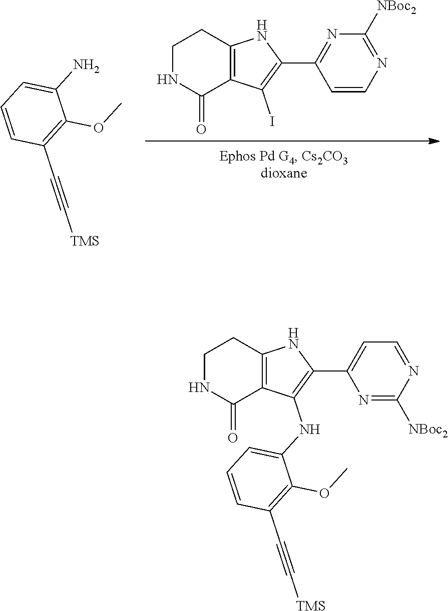

To a stirred solution of tert-butyl N-(tert-butoxycarbonyl)-N-(4-[3-iodo-4-oxo-1H,5H,6H,7H-pyrrolo[3,2-c] pyridin-2-yl]pyrimidin-2-yl)carbamate (200.00 mg, 0.36 mmol, 1.00 equiv) and 2-methoxy-3-[2-(trimethylsilyl)ethynyl]aniline (118.49 mg, 0.54 mmol, 1.50 equiv) in 1,4-dioxane was added Cs₂CO₃ (234.67 mg, 0.72 mmol, 2.00 equiv) and Ephos Pd G4 (66.16 mg, 0.07 mmol, 0.20 equiv) in one portion at room temperature under argon atmosphere. The resulting mixture was stirred for 2.5 h at 50° C. under argon atmosphere. The resulting mixture was concentrated under vacuum. The residue was purified by Prep-TLC (CH₂Cl₂/MeOH: 20:1) to afford tert-butyl N-(tert-butoxycarbonyl)-N-[4-[3-([2-methoxy-3-[2-(trimethylsilyl)ethynyl]phenyl]amino)-4-oxo-1H,5H,6H,7H-pyrrolo[3,2-c]pyridin-2-yl]pyrimidin-2-yl]carbamate (118.00 mg, 50.66%) as a yellow solid.

LC-MS: (M+H)⁺ found: 647.1.

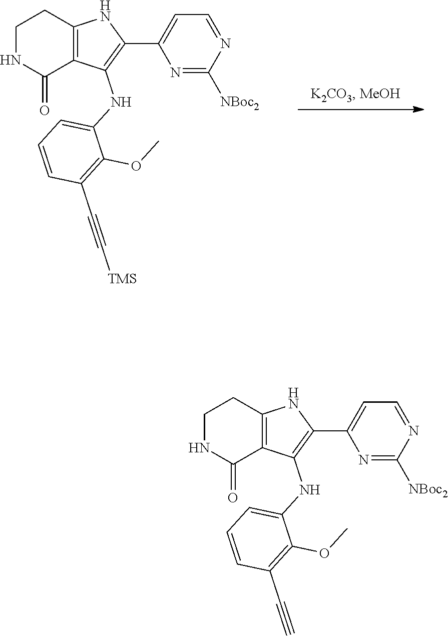

To a solution of tert-butyl N-(tert-butoxycarbonyl)-N-[4-[3-([2-methoxy-3-[2-(trimethylsilyl) ethynyl]phenyl]amino)-4-oxo-1H,5H,6H,7H-pyrrolo[3,2-c]pyridin-2-yl]pyrimidin-2-yl]carbamate (108.00 mg, 0.17 mol, 1.00 equiv) in MeOH (10 mL) was added K₂CO₃ (69.23 mg, 0.50 mmol, 3.00 equiv) at rt under Nitrogen atmosphere. Desired product could be detected by LC-MS after 1 h. The reaction was worked up with last batch (E10346-076). The resulting mixture was concentrated under vacuum. The residue was purified by Prep-TTLC (CH₂Cl₂/MeOH: 20:1) to afford tert-butyl N-(tert-butoxycarbonyl)-N-(4-[3-[(3-ethynyl-2-methoxyphenyl)amino]-4-oxo-1H,5H,6H,7H-pyrrolo[3,2-c]pyridin-2-yl]pyrimidin-2-yl)carbamate (99.00 mg, 103.18%) as orange solid.

LC-MS: (M+H)⁺ found: 575.1.

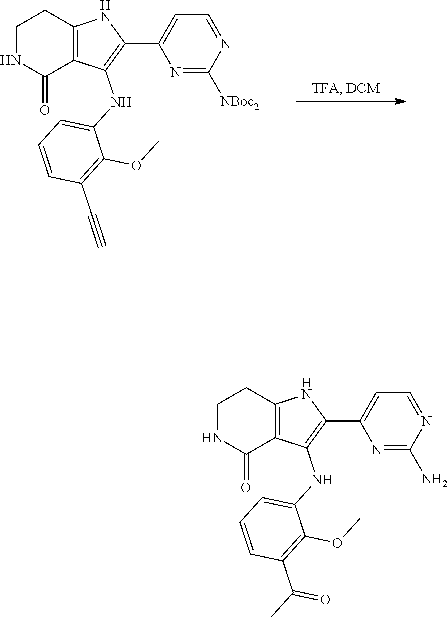

To a stirred solution of tert-butyl N-(tert-butoxycarbonyl)-N-(4-[3-[(3-ethynyl-2-methoxyphenyl)amino]-4-oxo-1H,5H,6H,7H-pyrrolo[3,2-c]pyridin-2-yl]pyrimidin-2-yl)carbamate (113.00 mg, 1.00 equiv) in DCM was added TFA (4.00 mL) dropwise at rt. The mixture was stirred for 2 h at rt. After start material was consumed, the resulting mixture was concentrated under vacuum. The crude product (80.00 mg) was purified by Prep-HPLC with the following conditions (NH₄HCO₃) to afford 3-[(3-acetyl-2-methoxyphenyl)amino]-2-(2-aminopyrimidin-4-yl)-1H,5H,6H,7H-pyrrolo[3,2-c]pyridin-4-one (7.70 mg) as a yellow solid.

LC-MS: (M+H)⁺ found: 393.1.

¹H NMR (300 MHz, DMSO-d₆) δ 11.65 (s, 1H), 8.05 (t, 2H), 7.15 (s, 1H), 6.96-6.88 (m, 2H), 6.61-6.59 (m, 1H), 6.46 (d, 1H), 6.19 (s, 2H), 3.90 (s, 3H), 3.41 (t, 2H), 2.85 (t, 2H), 2.57 (s, 3H).

### Example 131. 3-[(3-chloro-2-methoxyphenyl)amino]-2-(6,7-dimethoxy-1,5-naphthyridin-4-yl)-1H,5H,6H,7H-pyrrolo[3,2-c]pyridin-4-one (compound 730)

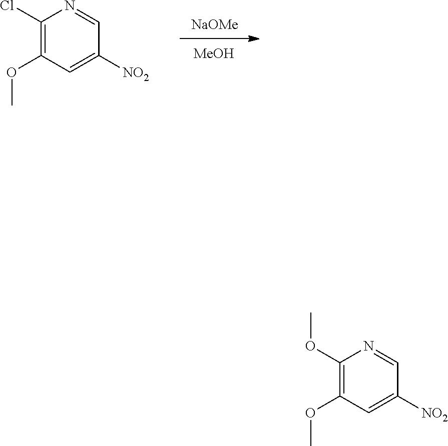

To a stirred solution 2-chloro-3-methoxy-5-nitropyridine (5.00 g, 26.52 mmol, 1.00 equiv) in methanol (80 mL) was added sodium methoxide (2.29 g, 42.42 mmol, 1.60 equiv) in portions at room temperature under nitrogen atmosphere. The resulting mixture was stirred for additional 1 h at room temperature. Desired product could be detected by LCMS. The resulting mixture was filtered, the filter cake was washed with MeOH (1×20 mL). This resulted in 2,3-dimethoxy-5-nitropyridine (4.63 g, 94.82%) as a yellow solid.

LC-MS: (M+H)⁺ found: 184.9.

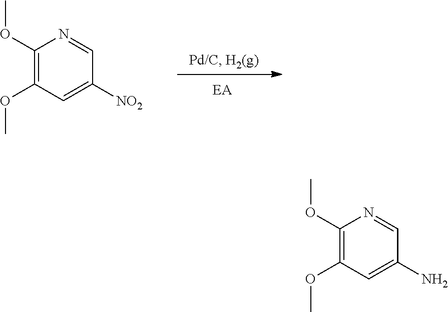

To a stirred mixture of 2,3-dimethoxy-5-nitropyridine (400.00 mg, 2.17 mmol, 1.00 equiv), Pd/C (231.16 mg, 0.22 mmol, 0.10 equiv, 10%) in EA (5 mL) in portions at room temperature under nitrogen atmosphere. The resulting mixture was stirred for overnight at room temperature under hydrogen atmosphere. The resulting mixture was filtered, the filter cake was washed with MeOH (3×30 mL). The filtrate was concentrated under reduced pressure. The residue was purified by silica gel column chromatography, eluted with CH₂Cl₂/MeOH (1:1) to 5,6-dimethoxypyridin-3-amine (318.00 mg, 94.96%) as a brown solid.

LC-MS: (M+H)⁺ found: 155.2.

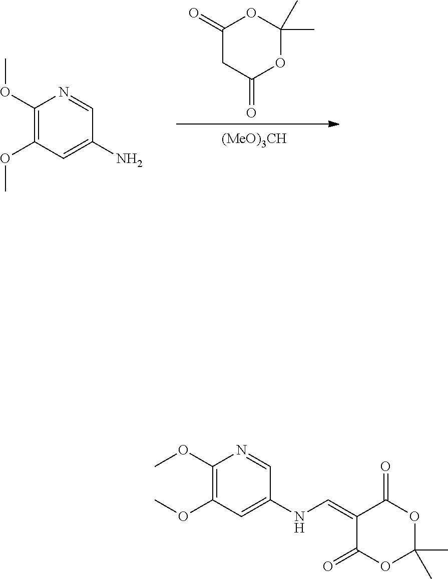

A mixture of 5,6-dimethoxypyridin-3-amine (300.00 mg, 1.95 mmol, 1.00 equiv) and meldrum's acid (280.46 mg, 1.95 mmol, 1.00 equiv) in trimethyl orthoformate (3 mL) was stirred for 2 h at 110° C. under air atmosphere. Desired product could be detected by LCMS. The mixture was allowed to cool down to room temperature. The resulting mixture was concentrated under reduced pressure to afford 5-[[(5,6-dimethoxypyridin-3-yl)amino]methylidene]-2,2-dimethyl-1,3-dioxane-4,6-dione (495.00 mg, 82.51%) as a brown solid.

LC-MS: (M+H)⁺ found: 309.1.

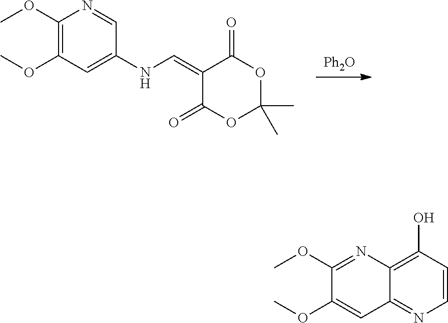

A solution of 5-{[(5,6-dimethoxypyridin-3-yl)amino]methylidene}-2,2-dimethyl-1,3-dioxane-4,6-dione (200.00 mg, 0.65 mmol, 1.00 equiv) in diphenyl-ether (5 mL) was stirred for 1 h at 250° C. under argon atmosphere. The mixture was allowed to cool down to room temperature. The resulting mixture was diluted with Et₂O (50 mL). The precipitated solids were collected by filtration and washed with Et₂O (2×100 mL). This resulted in 6,7-dimethoxy-1,5-naphthyridin-4-ol (80.00 mg, 59.80%) as a yellow solid.

LC-MS: (M+H)⁺ found: 207.1.

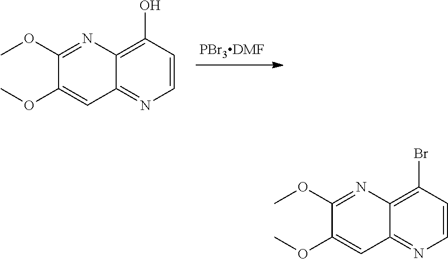

To a stirred solution/mixture of 6,7-dimethoxy-1,5-naphthyridin-4-ol (80.00 mg, 0.39 mmol, 1.00 equiv) in DMF (1 mL) was added PBr₃ (136.52 mg, 0.50 mmol, 1.30 equiv) in portions at 0° C. under nitrogen atmosphere. The resulting mixture was stirred for additional 2 h at room temperature. Desired product could be detected by LCMS. The mixture was allowed to cool down to 0° C. The reaction was quenched with Ice/Salt at 0° C. The aqueous layer was extracted with EtOAc (3×200 mL). The residue was purified by silica gel column chromatography, eluted with PE/EA (1:1) to afford 8-bromo-2,3-dimethoxy-1,5-naphthyridine (60.00 mg, 57.47%) as a yellow solid.

LC-MS: (M+H)⁺ found: 269.0.

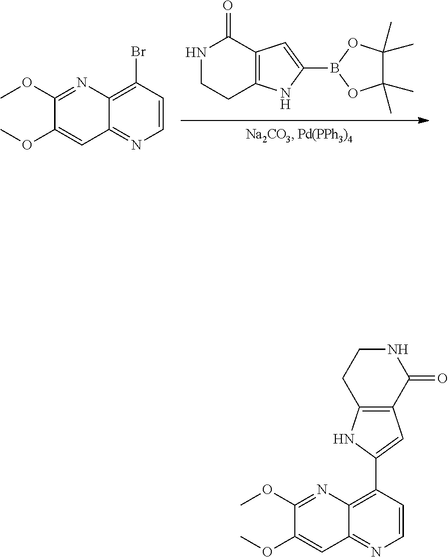

To a stirred mixture of 8-bromo-2,3-dimethoxy-1,5-naphthyridine (630.00 mg, 2.34 mmol, 1.00 equiv) and 2-(4,4,5,5-tetramethyl-1,3,2-dioxaborolan-2-yl)-1H,5H,6H,7H-pyrrolo[3,2-c]pyridin-4-one (1227.33 mg, 4.68 mmol, 2.00 equiv) in 1,4-dioxane (20 mL) and H₂O (4 mL) were added Na₂CO₃ (744.41 mg, 7.02 mmol, 3.00 equiv) and Pd(PPh₃)₄ (270.53 mg, 0.23 mmol, 0.10 equiv) in portions at room temperature under argon atmosphere. The resulting mixture was stirred for 2 h at 50° C. under argon atmosphere.

Desired product could be detected by LCMS. The resulting mixture was concentrated under reduced pressure. The residue was purified by silica gel column chromatography, eluted with CH₂C2/MeOH (20:1) to afford 2-(6,7-dimethoxy-1,5-naphthyridin-4-yl)-1H,5H,6H,7H-pyrrolo[3,2-c]pyridin-4-one (570.00 mg, 75.07%) as a yellow solid.

LC-MS: (M+H)⁺ found: 325.1.

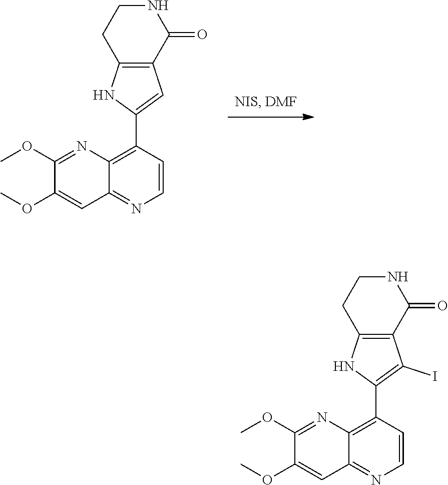 [SMILES: COc1cc2nccc(-c3cc4c([nH]3)CCNC4=O)c2nc1OC ; COc1cc2nccc(-c3[nH]c4c(c3I)C(=O)NCC4)c2nc1OC]

A mixture of 2-(6,7-dimethoxy-1,5-naphthyridin-4-yl)-1H,5H,6H,7H-pyrrolo[3,2-c]pyridin-4-one (200.00 mg, 0.62 mmol, 1.00 equiv) and NIS (208.10 mg, 0.35 mmol, 1.50 equiv) in DMF (2 mL) was stirred for 2 h at room temperature under nitrogen atmosphere. Desired product could be detected by LCMS. The resulting mixture was concentrated under reduced pressure. The residue was purified by silica gel column chromatography, eluted with CH₂Cl₂/MeOH (12:1) to afford 2-(6,7-dimethoxy-1,5-naphthyridin-4-yl)-3-iodo-1H,5H,6H,7H-pyrrolo[3,2-c]pyridin-4-one (190.00 mg, 68.44%) as a yellow solid.

LC-MS: (M+H)⁺ found: 450.9.

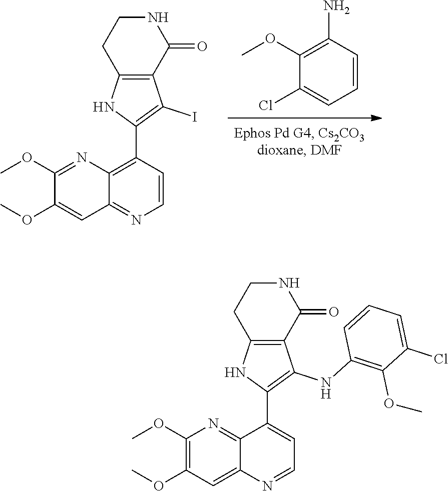

To a stirred mixture of 2-(6,7-dimethoxy-1,5-naphthyridin-4-yl)-3-iodo-1H,5H,6H,7H-pyrrolo[3,2-c]pyridin-4-one (160.00 mg, 0.36 mmol, 1.00 equiv) and 3-chloro-2-methoxyaniline (168.02 mg, 1.07 mmol, 3.00 equiv) in dioxane (2 mL) and DMF (2 mL) were added Cs₂CO₃ (231.57 mg, 0.71 mmol, 2.00 equiv) and Ephos Pd G4 (65.28 mg, 0.07 mmol, 0.20 equiv) in portions at room temperature under argon atmosphere. The resulting mixture was stirred for overnight at 50° C. under argon atmosphere. Desired product could be detected by LCMS. The resulting mixture was concentrated under reduced pressure. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 15% B to 39% B in 8 min, 39% B; Wave Length: 254/220 nm; RT1 (min): 7.9) to afford 3-[(3-chloro-2-methoxyphenyl)amino]-2-(6,7-dimethoxy-1,5-naphthyridin-4-yl)-1H,5H,6H,7H-pyrrolo[3,2-c]pyridin-4-one (9.80 mg, 5.71%) as an orange solid.

LC-MS: (M+H)⁺ found: 480.3.

¹H NMR (400 MHz, DMSO-d₆) δ 11.92 (s, 1H), 8.47 (d, J=4.8 Hz, 1H), 7.65 (s, 1H), 7.58 (s, 1H), 7.32 (d, J=5.2 Hz, 1H), 7.11 (s, 1H), 6.64-6.55 (m, 2H), 6.14-6.09 (m, 1H), 4.12 (s, 3H), 3.95 (s, 3H), 3.84 (s, 3H), 3.43 (s, 2H), 2.94 (t, J=6.8 Hz, 2H).

### Example 132. 3-[(3-chloro-2-methoxyphenyl)amino]-2-[6-(oxetan-3-yloxy)-1,5-naphthyridin-4-yl]-1H,5H,6H,7H-pyrrolo[3,2-c]pyridin-4-one (compound 732)

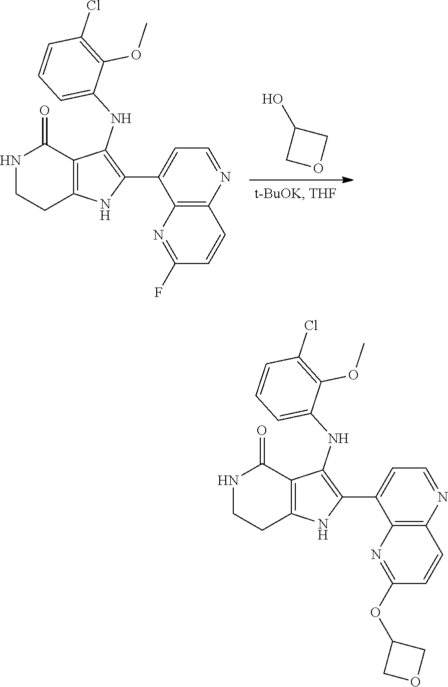 [SMILES: COc1c(Cl)cccc1Nc1c(-c2ccnc3ccc(F)nc23)[nH]c2c1C(=O)NCC2 ; OC1COC1 ; COc1c(Cl)cccc1Nc1c(-c2ccnc3ccc(OC4COC4)nc23)[nH]c2c1C(=O)NCC2]

To a solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (70.00 mg, 0.16 mmol, 1.00 equiv) and oxetan-3-ol (59.21 mg, 0.80 mmol, 5.00 equiv) in THF (1.60 mL) were added t-BuOK (53.80 mg, 0.48 mmol, 3.00 equiv) at 0° C. and stirred for 0.5 h. The resulting mixture was concentrated under reduced pressure. The crude product was purified by Prep-HPLC with the following conditions (Column: XBridge Prep OBD C18 Column, 30*150 mm, 5|Ìm; Mobile Phase A: Water (10 mmol/L NH₄HCO₃), Mobile Phase B: ACN; Flow rate: 60 mL/min; Gradient: 29% B to 59% B in 7 min; Wave Length: 254 nm; RT1 (min): 6.5) to afford 3-[(3-chloro-2-methoxyphenyl)amino]-2-[6-(oxetan-3-yloxy)-1,5-naphthyridin-4-yl]-1H,5H,6H,7H-pyrrolo[3,2-c]pyridin-4-one (27.20 mg, 34.38%) as a yellow solid.

LC-MS: (M+H)⁺ found: 492.0.

¹H NMR (400 MHz, DMSO-d₆): δ 11.71 (s, 1H), 8.61 (d, J=4.8 Hz, 1H), 8.66 (d, J=9.1 Hz, 1H), 7.65 (s, 1H), 7.52 (d, J=4.8 Hz, 1H), 7.43 (d, J=9.0 Hz, 1H), 7.19 (t, J=2.6 Hz, 1H), 6.72-6.55 (m, 2H), 6.13 (dd, J=7.8, 1.9 Hz, 1H), 6.09-5.95 (m, 1H), 5.03-4.86 (m, 2H), 4.72 (dd, J=7.6, 5.1 Hz, 2H), 3.85 (s, 3H), 3.50 (td, J=6.9, 2.6 Hz, 2H), 2.99 (t, J=6.8 Hz, 2H).

### Example 133. 3-[(3-chloro-2-methoxyphenyl) amino]-2-[6-(2,2-difluoroethoxy)-1,5-naphthyridin-4-yl]-1H,5H,6H,7H-pyrrolo[3,2-c]pyridin-4-one (compound 733)

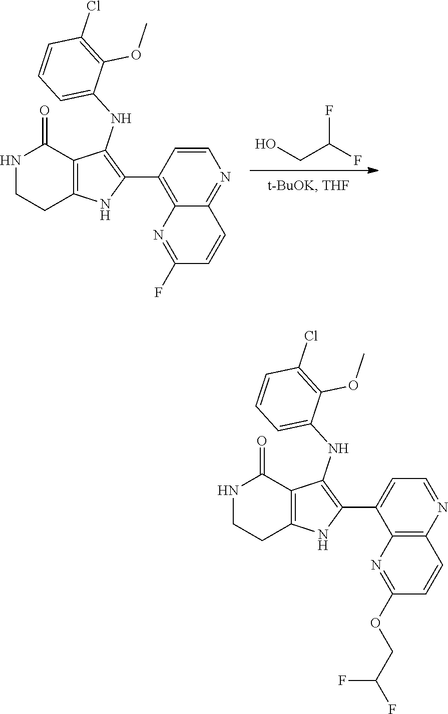 [SMILES: COc1c(Cl)cccc1Nc1c(-c2ccnc3ccc(F)nc23)[nH]c2c1C(=O)NCC2 ; OCC(F)F ; COc1c(Cl)cccc1Nc1c(-c2ccnc3ccc(OCC(F)F)nc23)[nH]c2c1C(=O)NCC2]

A solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (50.00 mg, 0.11 mmol, 1.00 equiv) in THF (1.00 mL) was added 2,2-difluoroethanol (47.00 mg, 0.57 mmol, 5.00 equiv) and t-BuOK (38.00 mg, 0.34 mmol, 3.00 equiv) dropwise at 0° C. The resulting mixture was stirred for 1 h at room temperature. The reaction was monitored by LCMS. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 36% B to 66% B in 7 min, 66% B; Wave Length: 254 nm; RT1 (min): 6.5) to afford 3-[(3-chloro-2-methoxyphenyl) amino]-2-[6-(2,2-difluoroethoxy)-1,5-naphthyridin-4-yl]-1H,5H,6H,7H-pyrrolo[3,2-c]pyridin-4-one (29.60 mg, 59.36%) as a yellow solid.

LC-MS: (M+H)⁺ found: 500.0.

¹H NMR (400 MHz, DMSO-d₆) δ 11.80 (s, 1H), 8.62 (d, J=4.8 Hz, 1H), 8.36 (d, J=9.2 Hz, 1H), 7.76 (s, 1H), 7.52 (d, J=4.8 Hz, 1H), 7.44 (d, J=8.8 Hz, 1H), 7.20 (s, 1H), 6.69-6.55 (m, 3H), 6.17-6.15 (m, 1H), 4.94-4.86 (m, 2H), 3.88 (s, 3H), 3.48-3.46 (m, 2H), 2.93 (t, J=6.8 Hz, 2H).

### Example 134. 3-[(3-chloro-2-methoxyphenyl)amino]-2-[6-[2-(dimethylamino)ethoxy]-1,5-naphthyridin-4-yl]-1H,5H,6H,7H-pyrrolo[3,2-c] pyridin-4-one; formic acid (compound 736)

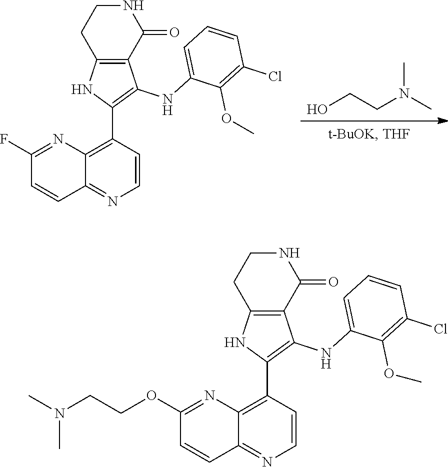

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (80.00 mg, 0.18 mmol, 1.00 equiv) and dimethylaminoethanol (81.00 mg, 0.91 mmol, 5.00 equiv) in THF (4 mL) was added t-BuOK (62.00 mg, 0.55 mmol, 3.00 equiv) in portions at 0° C. under nitrogen atmosphere. The resulting mixture was stirred for 1 h at room temperature under nitrogen atmosphere. Desired product could be detected by LCMS. The reaction was quenched by the addition of Water (1 mL) at 0° C. The resulting mixture was concentrated under vacuum. The crude product was purified by Prep-HPLC with the following conditions (Column. Xselect CSH OBD Column 30*150 mm 5 um, n; Mobile Phase A: Water (0.1% FA), Mobile Phase B: ACN; Flow rate: 60 mL/min; Gradient: 11% B to 24% B in 8 min, 24% B; Wave Length: 254/220 nm; RT1 (min): 7.18) to afford 3-[(3-chloro-2-methoxyphenyl)amino]-2-[6-[2-(dimethylamino)ethoxy]-1,5-naphthyridin-4-yl]-1H,5H,6H,7H-pyrrolo[3,2-c] pyridin-4-one; formic acid (24.80 mg, 24.05%) as an orange solid.

LC-MS: (M+H)⁺ found: 507.1.

¹H NMR (400 MHz, DMSO-d₆) δ 12.04 (s, 1H), 8.58 (d, J=4.8 Hz, 1H), 8.29 (d, J=9.0 Hz, 1H), 7.74 (s, 1H), 7.52 (d, J=4.9 Hz, 1H), 7.34 (d, J=9.1 Hz, 1H), 7.21 (t, J=2.6 Hz, 1H), 6.72-6.61 (m, 2H), 6.21-6.15 (m, 1H), 4.67 (t, J=6.0 Hz, 2H), 3.86 (s, 3H), 3.50-3.44 (m, 2H), 2.93 (t, J=6.8 Hz, 2H), 2.77 (t, J=6.0 Hz, 2H), 2.27 (s, 6H).

### Example 135. 3-[(3-chloro-2-methoxyphenyl)amino]-2-[6-(oxetan-3-ylmethoxy)-1,5-naphthyridin-4-yl]-1H,5H,6H,7H-pyrrolo[3,2-c] pyridin-4-one (compound 737)

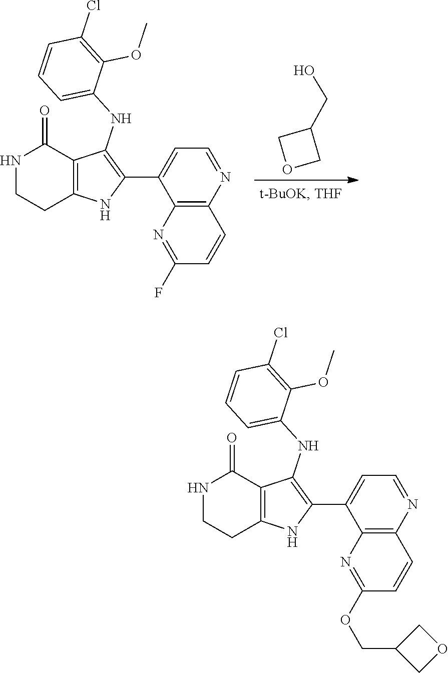 [SMILES: COc1c(Cl)cccc1Nc1c(-c2ccnc3ccc(F)nc23)[nH]c2c1C(=O)NCC2 ; OCC1COC1 ; COc1c(Cl)cccc1Nc1c(-c2ccnc3ccc(OCC4COC4)nc23)[nH]c2c1C(=O)NCC2]

To a stirred solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (100.00 mg, 0.23 mmol, 1.00 equiv) in THF (3.00 mL) were added oxetan-3-ylmethanol (100.61 mg, 1.14 mmol, 5.00 equiv) and t-BuOK (76.88 mg, 0.69 mmol, 3.00 equiv) in portions at degrees 0° C. under argon atmosphere. The resulting mixture was stirred for 1 h at room temperature under argon atmosphere. The crude product (100.00 mg) was purified by Prep-HPLC with the following conditions (Column: XBridge Prep C18 OBD Column, 19*150 mm, 5 μm; Mobile Phase A: Water (10 mmol/L NH₄HCO₃), Mobile Phase B: ACN; Flow rate: 60 mL/min; Gradient: 28% B to 48% B in 10 min, 48% B; Wave Length: 254/220 nm; RT1 (min): 9.28) to afford 3-[(3-chloro-2-methoxyphenyl)amino]-2-[6-(oxetan-3-ylmethoxy)-1,5-naphthyridin-4-yl]-1H,5H,6H,7H-pyrrolo[3,2-c] pyridin-4-one (29.70 mg, 25.19%) as a yellow solid.

LC-MS: (M+H)⁺ found: 506.0.

¹H NMR (300 MHz, DMSO-d₆) δ 12.07 (s, 1H), 8.59 (d, J=4.8 Hz, 1H), 8.30 (d, J=9.1 Hz, 1H), 7.77 (s, 1H), 7.52 (d, J=4.8 Hz, 1H), 7.36 (d, J=9.1 Hz, 1H), 7.22 (s, 1H), 6.75-6.60 (m, 2H), 6.15-6.13 (m, 1H), 4.87-4.73 (m, 4H), 4.53 (t, J=6.0 Hz, 2H), 3.89 (s, 3H), 3.62-3.43 (m, 3H), 2.96 (t, J=6.7 Hz, 2H).

### Example 136. 3-[(3-chloro-2-methoxyphenyl)amino]-2-[6-[(1-methylazetidin-3-yl)methoxy]-1,5-naphthyridin-4-yl]-1H,5H,6H,7H-pyrrolo[3,2-c]pyridin-4-one (compound 738)

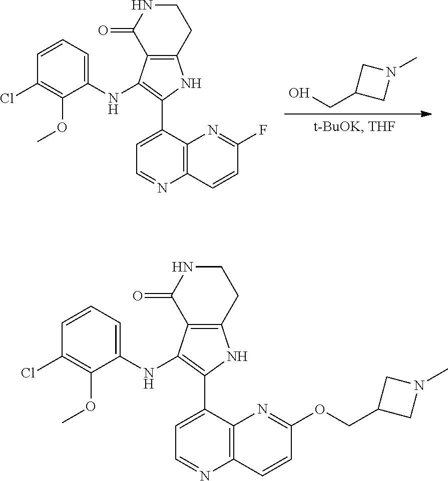

To a stirred solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (40.00 mg, 0.09 mmol, 1.00 equiv) and (1-methylazetidin-3-yl)methanol (92.00 mg, 0.91 mmol, 10.00 equiv) in THF (1.00 mL) were added t-BuOK (31.00 mg, 0.27 mmol, 3.00 equiv) in portions at 0° C. under N₂ atmosphere. The resulting mixture was stirred for 1 h at rt under N₂ atmosphere. The resulting mixture was concentrated under reduced pressure. The resulting mixture was concentrated under vacuum and dissolved in DMF. The crude product (100.00 mg) was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 11% B to 25% B in 8 min, 25% B; Wave Length: 254/220 nm; RT1 (min): 7.37) to afford 3-[(3-chloro-2-methoxyphenyl)amino]-2-[6-[(1-methylazetidin-3-yl)methoxy]-1,5-naphthyridin-4-yl]-1H,5H,6H,7H-pyrrolo[3,2-c]pyridin-4-one (15.90 mg, 33.54%) as a yellow solid.

LC-MS: (M+H)⁺ found: 519.1.

¹H NMR (300 MHz, DMSO-d₆) δ 12.08 (s, 1H), 8.58 (d, J=4.8 Hz, 1H), 8.35-8.18 (m, 1H), 7.74 (s, 1H), 7.52 (d, J=4.9 Hz, 1H), 7.34 (d, J=9.1 Hz, 1H), 7.19 (d, J=2.7 Hz, 1H), 6.75-6.54 (m, 2H), 6.17 (m, J=7.7, 2.0 Hz, 1H), 4.73 (d, J=7.0 Hz, 2H), 3.83 (s, 3H), 3.48 (m, J=6.4, 3.3 Hz, 2H), 3.35 (s, 2H), 3.09 (t, J=6.5 Hz, 2H), 2.92 (m, J=12.4, 6.7 Hz, 3H), 2.26 (s, 3H).

### Example 137. 3-[(3-chloro-2-methoxyphenyl) amino]-2-[6-[(3S)-oxolan-3-yloxy]-1,5-naphthyridin-4-yl]-1H,5H,6H,7H-pyrrolo[3,2-c]pyridin-4-one (compound 739)

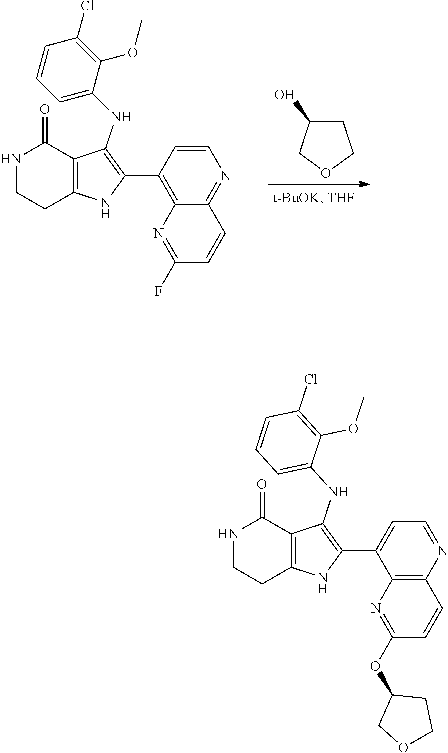 [SMILES: COc1c(Cl)cccc1Nc1c(-c2ccnc3ccc(F)nc23)[nH]c2c1C(=O)NCC2 ; O[C@H]1CCOC1 ; COc1c(Cl)cccc1Nc1c(-c2ccnc3ccc(O[C@H]4CCOC4)nc23)[nH]c2c1C(=O)NCC2]

A solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (60.00 mg, 0.18 mmol, 1.00 equiv) in THF (1.50 mL) was added (3S)-oxolan-3-ol (60.00 mg, 0.90 mmol, 5.00 equiv) and t-BuOK (46.00 mg, 0.41 mmol, 3.00 equiv) dropwise at 0° C. The resulting mixture was stirred for 1 h at room temperature. The reaction was monitored by LCMS. The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 33% B to 63% B in 7 min; Wave Length: 254 nm; RT1 (min): 6.5) to afford 3-[(3-chloro-2-methoxyphenyl) amino]-2-[6-[(3S)-oxolan-3-yloxy]-1,5-naphthyridin-4-yl]-1H,5H,6H,7H-pyrrolo[3,2-c]pyridin-4-one (15.40 mg, 22.06%) as a yellow solid.

LC-MS: (M+H)⁺ found: 447.0.

¹H NMR (400 MHz, DMSO-d₆) δ 11.96 (s, 1H), 8.58 (d, J=4.8 Hz, 1H), 8.30 (d, J=8.8 Hz, 1H), 7.70 (s, 1H), 7.51 (d, J=4.8 Hz, 1H), 7.33 (d, J=9.2 Hz, 1H), 7.20 (s, 1H), 6.69-6.61 (m, 2H), 6.17-6.14 (m, 1H), 5.84-5.83 (m, 1H), 4.04-3.93 (m, 3H), 3.91 (s, 3H), 3.88-3.81 (m, 1H), 3.48-3.45 (m, 2H), 2.95 (t, J=6.8 Hz, 2H), 2.44-2.29 (m, 1H), 2.28-2.17 (m, 1H).

### Example 138. 3-[(3-chloro-2-methoxyphenyl)amino]-2-[6-(oxan-4-yloxy)-1,5-naphthyridin-4-yl]-1H,5H,6H,7H-pyrrolo[3,2-c]pyridin-4-one (compound 740)

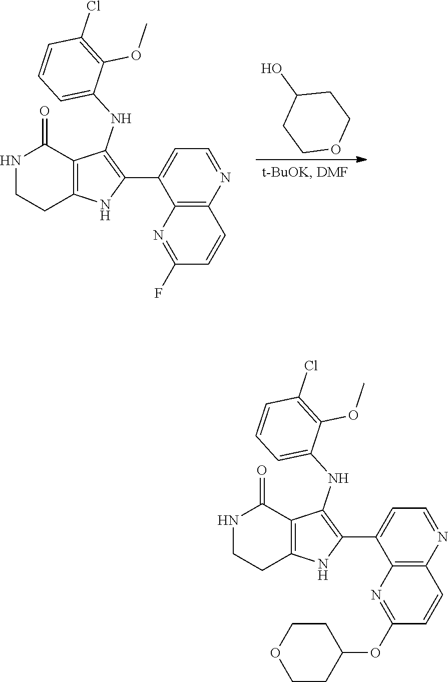 [SMILES: COc1c(Cl)cccc1Nc1c(-c2ccnc3ccc(F)nc23)[nH]c2c1C(=O)NCC2 ; OC1CCOCC1 ; COc1c(Cl)cccc1Nc1c(-c2ccnc3ccc(OC4CCOCC4)nc23)[nH]c2c1C(=O)NCC2]

To a solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (100.00 mg, 0.23 mmol, 1.00 equiv) and oxan-4-ol (116.63 mg, 1.14 mmol, 5.00 equiv) in DMF (2.00 mL) were added t-BuOK (28.19 mg, 0.25 mmol, 1.10 equiv) dropwise at 0° C. under nitrogen atmosphere. The resulting mixture was stirred for 3 h at room temperature under nitrogen atmosphere. The mixture was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 28% B to 45% B in 8 min, 45% B; Wave Length: 254/220 nm; RT1 (min): 7.58) to afford 3-[(3-chloro-2-methoxyphenyl)amino]-2-[6-(oxan-4-yloxy)-1,5-naphthyridin-4-yl]-1H,5H,6H,7H-pyrrolo[3,2-c]pyridin-4-one (18.40 mg, 14.72%) as a red solid.

LC-MS: (M+H)⁺ found: 520.1.

¹H NMR (400 MHz, DMSO-d₆): δ 11.90 (s, 1H), 8.57 (d, J=4.8 Hz, 1H), 8.29 (d, J=9.0 Hz, 1H), 7.72 (s, 1H), 7.51 (d, J=4.8 Hz, 1H), 7.32 (d, J=9.1 Hz, 1H), 7.25-7.17 (m, 1H), 6.75-6.56 (m, 2H), 6.13 (dd, J=8.0, 1.7 Hz, 1H), 5.60-5.44 (m, 1H), 4.01-3.92 (m, 2H), 3.90 (s, 3H), 3.62-3.43 (m, 4H), 3.02-2.91 (m, 2H), 2.24-2.13 (m, 2H), 1.87-1.69 (m, 2H).

### Example 139. 3-[(3-chloro-2-methoxyphenyl)amino]-2-[6-[(3S)-oxan-3-yloxy]-1,5-naphthyridin-4-yl]-1H,5H,6H,7H-pyrrolo[3,2-c]pyridin-4-one; formic acid (compound 741)

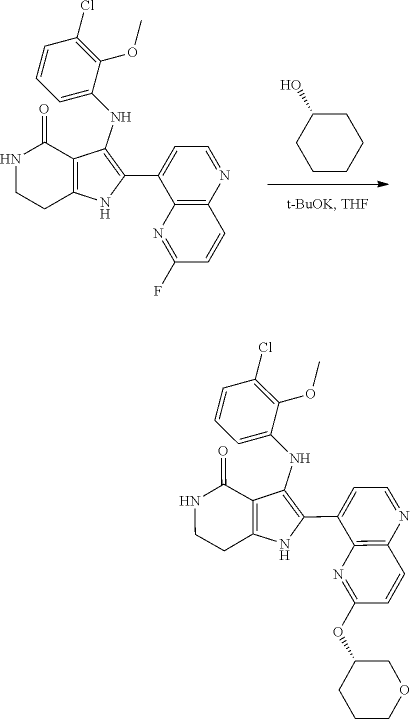

To a stirred solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (70.00 mg, 0.16 mmol, 1.00 equiv) in THF (2.00 mL) were added t-BuOK (35.88 mg, 0.32 mmol, 2.00 equiv) in portions at 0° C. under nitrogen atmosphere for 0.5 h. The crude product (100.00 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 18% B to 48% B in 10 min, 48% B; Wave Length: 220/254 nm; RT1 (min): 9.35) to afford 3-[(3-chloro-2-methoxyphenyl)amino]-2-[6-[(3S)-oxan-3-yloxy]-1,5-naphthyridin-4-yl]-1H,5H,6H,7H-pyrrolo[3,2-c]pyridin-4-one; formic acid (13.50 mg, 14.17%) as a yellow solid.

LC-MS: (M+H)⁺ found: 520.1.

¹H NMR (400 MHz, DMSO-d₆) δ 11.97 (s, 1H), 8.57 (d, J=4.8 Hz, 1H), 8.30 (d, J=9.1 Hz, 1H), 8.21 (s, 1H), 7.69 (s, 1H), 7.50 (d, J=4.8 Hz, 1H), 7.33 (d, J=9.1 Hz, 1H), 7.22 (d, J=2.7 Hz, 1H), 6.72-6.61 (m, 2H), 6.15 (dd, J=7.9, 1.8 Hz, 1H), 5.31 (tt, J=7.1, 3.6 Hz, 1H), 4.07-3.99 (m, 1H), 3.89 (s, 3H), 3.69 (ddd, J=14.5, 11.1, 5.1 Hz, 2H), 3.59 (ddd, J=11.0, 7.5, 3.3 Hz, 1H), 3.47 (td, J=6.9, 2.5 Hz, 1H), 2.95 (t, J=6.8 Hz, 2H), 2.21 (td, J=8.1, 2.6 Hz, 1H), 1.98-1.81 (m, 2H), 1.64-1.60 (m, 1H).

### Example 140. 3-[(3-chloro-2-methoxyphenyl)amino]-2-[6-[(3R)-oxan-3-yloxy]-1,5-naphthyridin-4-yl]-1H,5H,6H,7H-pyrrolo[3,2-c]pyridin-4-one; formic acid (compound 742)

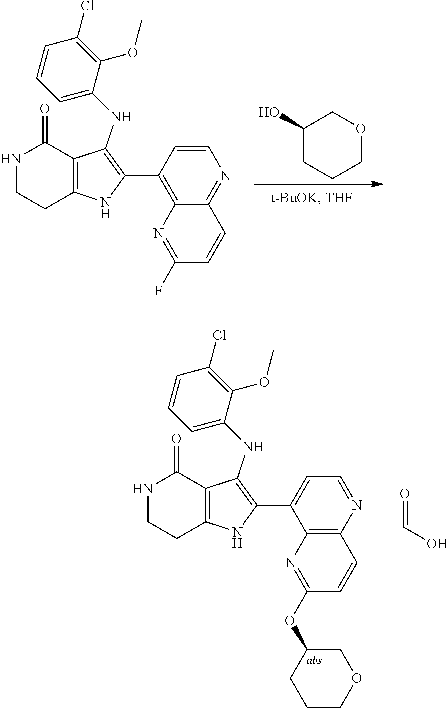 [SMILES: COc1c(Cl)cccc1Nc1c(-c2ccnc3ccc(F)nc23)[nH]c2c1C(=O)NCC2 ; O[C@@H]1CCCOC1 ; COc1c(Cl)cccc1Nc1c(-c2ccnc3ccc(O[C@@H]4CCCOC4)nc23)[nH]c2c1C(=O)NCC2 ; O=CO]

A solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (50.00 mg, 0.11 mmol, 1.00 equiv) in THF (2.00 mL) was added (3R)-oxan-3-ol (58.00 mg, 0.55 mmol, 5.00 equiv) and t-BuOK (38.00 mg, 0.33 mmol, 3.00 equiv) dropwise at 0° C. The resulting mixture was stirred for 1 h at RT. The reaction was monitored by LCMS. The crude product was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 30% B to 46% B in 8 min, 46% B; Wave Length: 254/220 nm; RT1 (min): 7.8) to afford 3-[(3-chloro-2-methoxyphenyl)amino]-2-[6-[(3R)-oxan-3-yloxy]-1,5-naphthyridin-4-yl]-1H,5H,6H,7H-pyrrolo[3,2-c]pyridin-4-one; formic acid (10.00 mg, 15.22%) as a yellow solid.

LC-MS: (M+H)⁺ found: 566.0.

¹H NMR (400 MHz, DMSO-d₆) δ 11.97 (s, 1H), 8.57 (d, J=4.8 Hz, 1H), 8.30-8.25 (m, 2H), 7.69 (s, 1H), 7.49 (d, J=4.8 Hz, 1H), 7.32 (d, J=9.2 Hz, 1H), 7.21 (s, 1H), 6.70-6.62 (m, 2H), 6.16-6.13 (m, 1H), 5.31-5.30 (m, 1H), 4.04-4.00 (m, 1H), 3.89 (s, 3H), 3.69-3.60 (m, 2H), 3.59-3.57 (m, 1H), 3.48-3.45 (m, 2H), 2.94 (t, J=6.8 Hz, 2H), 2.21-2.18 (m, 1H), 2.08 (s, 1H), 1.93-1.85 (m, 2H), 1.67-1.56 (m, 1H).

### Example 141. 3-[(3-chloro-2-methoxyphenyl)amino]-2-[6-[(3R)-oxolan-3-yloxy]-1,5-naphthyridin-4-yl]-1H,5H,6H,7H-pyrrolo[3,2-c]pyridin-4-one (compound 743)

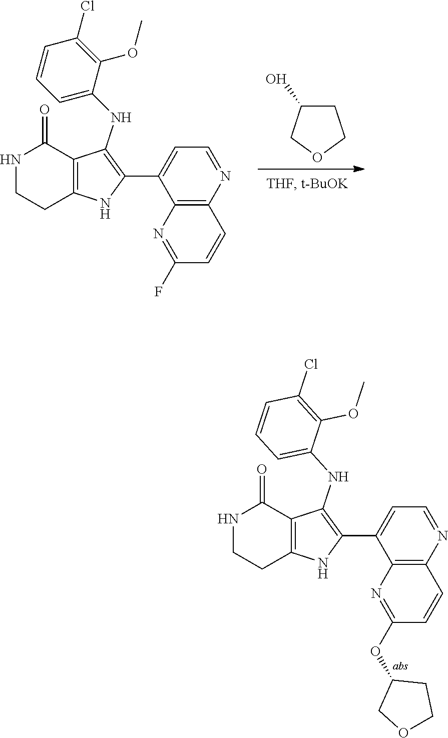 [SMILES: COc1c(Cl)cccc1Nc1c(-c2ccnc3ccc(F)nc23)[nH]c2c1C(=O)NCC2 ; O[C@@H]1CCOC1 ; COc1c(Cl)cccc1Nc1c(-c2ccnc3ccc(O[C@@H]4CCOC4)nc23)[nH]c2c1C(=O)NCC2]

To a stirred solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (100.00 mg, 0.23 mmol, 1.00 equiv) and (3R)-oxolan-3-ol (20.12 mg, 0.23 mmol, 1.00 equiv) in THF (3 mL) was added t-BuOK (76.88 mg, 0.69 mmol, 3.00 equiv) dropwise at 0° C. under nitrogen atmosphere. The solution was stirred for 3 h at room temperature under nitrogen atmosphere. Desired product could be detected by LCMS. The resulting mixture was concentrated under reduced pressure and dissolved in DMF. The residue was purified by silica gel column with the following conditions (column, silica gel; mobile phase, DCM in MeOH, 0% to 10% gradient in 10 min; detector, UV 254 nm) to afford crude product (100.00 mg). Then the crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 26% B to 56% B in 7 min, 56% B; Wave Length: 254 nm; RT1 (min): 6.5) to afford 3-[(3-chloro-2-methoxyphenyl)amino]-2-[6-[(3R)-oxolan-3-yloxy]-1,5-naphthyridin-4-yl]-1H,5H,6H,7H-pyrrolo[3,2-c]pyridin-4-one (4.80 mg, 4.10%) as a yellow solid.

LC-MS: (M+H)⁺ found: 506.2.

¹H NMR (400 MHz, DMSO-d₆) δ 11.96 (s, 1H), 8.59 (d, J=4.9 Hz, 1H), 8.29 (s, 1H), 7.70 (s, 1H), 7.51 (d, J=4.8 Hz, 1H), 7.33 (d, J=9.1 Hz, 1H), 7.20 (s, 1H), 6.72-6.49 (m, 2H), 6.29-6.08 (m, 1H), 5.85 (s, 1H), 4.00-3.83 (m, 7H), 3.47 (q, J=6.2, 5.6 Hz, 2H), 2.96 (t, J=6.8 Hz, 2H), 2.39-2.07 (m, 2H).

### Example 142. 3-[(3-chloro-2-methoxyphenyl)amino]-2-(6-methyl-1,5-naphthyridin-4-yl)-1H,5H,6H,7H-pyrrolo[3,2-c]pyridin-4-one (compound 749)

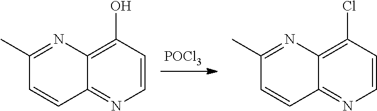

A stirred solution of 6-methyl-1,5-naphthyridin-4-ol (300.00 mg, 1.00 equiv) in POCl₃ (5.00 mL) was stirred for 1 h to reflux under nitrogen atmosphere. Desired product could be detected by LCMS. The resulting mixture was concentrated under vacuum and basified with saturated NaHCO₃ (aq.) to give 8-chloro-2-methyl-1,5-naphthyridine (170.00 mg) as off-white solid.

LC-MS: (M+H)⁺ found: 179.0.

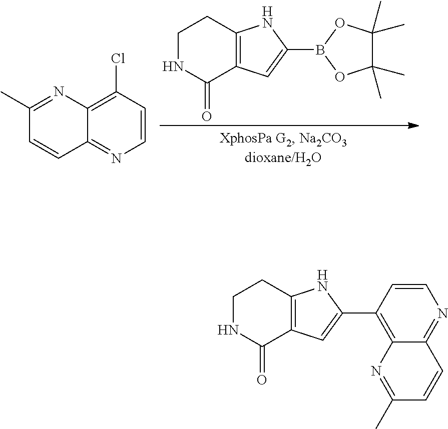 [SMILES: Cc1ccc2nccc(Cl)c2n1 ; CC1(C)OB(c2cc3c([nH]2)CCNC3=O)OC1(C)C ; Cc1ccc2nccc(-c3cc4c([nH]3)CCNC4=O)c2n1]

To a stirred solution/mixture of 8-chloro-2-methyl-1,5-naphthyridine (170.00 mg, 1.68 mmol, 1.00 equiv) and 2-(4,4,5,5-tetramethyl-1,3,2-dioxaborolan-2-yl)-1H,5H,6H,7H-pyrrolo[3,2-c]pyridin-4-one (660.36 mg, 2.52 mmol, 1.50 equiv) in dioxane (0.50 mL) and H₂O (0.10 mg) was added Na₂CO₃ (356.02 mg, 3.36 mmol, 2.00 equiv) and XPhos-PdCl-2nd G (255.21 mg, 0.34 mmol, 0.20 equiv) in portions at room temperature under argon atmosphere. The resulting mixture was stirred for 3 h at 60° C. under argon atmosphere. The resulting mixture was concentrated under vacuum. The resulting mixture was diluted with water (5 mL) and stirred for 20 min. The precipitated solids were collected by filtration and washed with water (2×3 mL). The solid was washed with DCM (10 mL) and filtered to give 2-(6-methyl-1,5-naphthyridin-4-yl)-1H,5H,6H,7H-pyrrolo[3,2-c]pyridin-4-one (130.00 mg, 68.46%) as brown solid which was used directly in next step.

LC-MS: (M+H)⁺ found: 279.0.

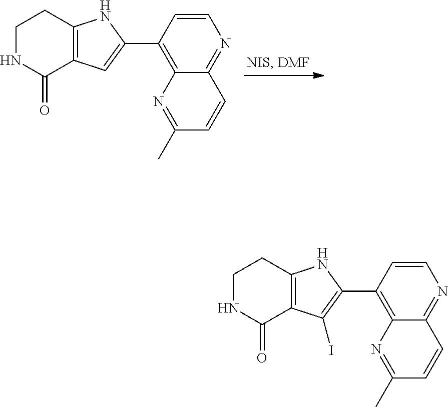 [SMILES: Cc1ccc2nccc(-c3cc4c([nH]3)CCNC4=O)c2n1 ; Cc1ccc2nccc(-c3[nH]c4c(c3I)C(=O)NCC4)c2n1]

To a stirred solution of 2-(6-methyl-1,5-naphthyridin-4-yl)-1H,5H,6H,7H-pyrrolo[3,2-c]pyridin-4-one (130.00 g, 0.47 mmol, 1.00 equiv) in DMF was added NIS (126.00 mg, 0.56 mmol, 1.20 equiv) in 2 portions at room temperature under air. The resulting mixture was stirred for 2 h at room temperature under air. Desired product could be detected in LC-MS. The reaction was quenched with Na₂SO₃ (aq.) at room temperature. The resulting mixture was filtered, the filter cake was washed with water (3×10 mL) and DCM (5 mL). The filtrate was concentrated under reduced pressure to give 3-iodo-2-(6-methyl-1,5-naphthyridin-4-yl)-1H,5H,6H,7H-pyrrolo[3,2-c]pyridin-4-one (140.00 mg, 74.05%).

LC-MS: (M+H)⁺ found: 405.0.

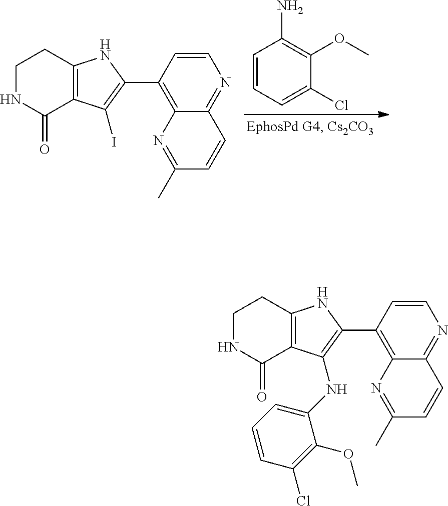

To a stirred solution of 3-iodo-2-(6-methyl-1,5-naphthyridin-4-yl)-1H,5H,6H,7H-pyrrolo[3,2-c]pyridin-4-one (100.00 mg, 0.25 mmol, 1.00 equiv) and 3-chloro-2-methoxyaniline (58.48 mg, 0.37 mmol, 1.50 equiv) in 1,4-dioxane were added EPhos Pd G4 (45.45 mg, 0.05 mmol, 0.20 equiv) and Cs₂CO₃ (161.21 mg, 0.50 mmol, 2.00 equiv) in portions at room temperature under argon atmosphere. The resulting mixture was stirred for 4 h at 50° C. under argon atmosphere. Desired product could be detected by LC-MS. The resulting mixture was concentrated under reduced pressure. The residue was purified by Prep-TLC (CH₂Cl₂/MeOH 20:1) and HPLC (Column: XBridge Prep OBD C18 Column, 30*150 mm, 51m; Mobile Phase A: Water (10 mmol/L NH₄HCO₃), Mobile Phase B: ACN; Flow rate: 60 mL/min; Gradient: 34% B to 64% B in 7 min; Wave Length: 254 nm; RT1 (min): 6.5) to afford 3-[(3-chloro-2-methoxyphenyl)amino]-2-(6-methyl-1,5-naphthyridin-4-yl)-1H,5H,6H,7H-pyrrolo[3,2-c]pyridin-4-one (13.20 mg, 14.35%) as a yellow solid.

LC-MS: (M+H)⁺ found: 434.0.

¹H NMR (300 MHz, CD₃OD) δ 8.54 (d, 1H), 8.26 (d, 1H), 7.71 (d, 1H), 7.61 (d, 1H), 6.71 (d, 1H), 6.65 (d, 1H), 6.23 (m, 1H), 4.01 (s, 3H), 3.34 (t, 2H), 3.29 (t, 2H), 2.90 (s, 3H).

### Example 143. 3-[(3-chloro-2-methoxyphenyl)amino]-2-[furo[3,2-b]pyridin-7-yl]-1H,5H,6H,7H-pyrrolo[3,2-c]pyridin-4-one (compound 750)

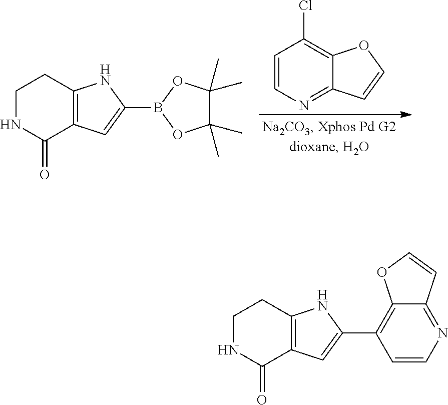

A mixture of 7-chlorofuro[3,2-b]pyridine (400.00 mg, 2.60 mmol, 1.00 equiv), 2-(4,4,5,5-tetramethyl-1,3,2-dioxaborolan-2-yl)-1H,5H,6H,7H-pyrrolo[3,2-c]pyridin-4-one (1.02 g, 3.91 mmol, 1.50 equiv) and Na₂CO₃ (323.00 mg, 3.05 mmol, 2.00 equiv) in dioxane (15 mL) and water (3 mL) at room temperature under nitrogen atmosphere. To the above mixture was added 2nd Generation XPhos precatalyst (232.00 mg, 0.31 mmol, 0.20 equiv) under nitrogen atmosphere. The resulting mixture was stirred for additional overnight at 50° C. The resulting mixture was extracted with CH₂Cl₂ (3×300 mL). The combined organic layers were washed with water (3×50 mL), dried over anhydrous Na₂SO₄. After filtration, the filtrate was concentrated under reduced pressure. The residue was purified by Prep-TLC (CH₂Cl₂/MeOH 10:1) to afford 2-[furo[3,2-b]pyridin-7-yl]-1H,5H,6H,7H-pyrrolo[3,2-c]pyridin-4-one (430.00 mg, 76.66%) as a brown solid.

LC-MS: (M+H)⁺ found: 254.0.

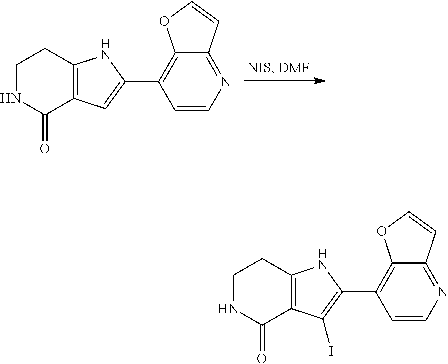 [SMILES: O=C1NCCc2[nH]c(-c3ccnc4ccoc34)cc21 ; O=C1NCCc2[nH]c(-c3ccnc4ccoc34)c(I)c21]

To a stirred mixture of 2-[furo[3,2-b]pyridin-7-yl]-1H,5H,6H,7H-pyrrolo[3,2-c]pyridin-4-one (150.00 mg, 0.59 mmol, 1.00 equiv) in DMF (6.00 mL) was added NIS (160.00 mg, 0.71 mmol, 1.20 equiv) in three portions at room temperature under nitrogen atmosphere. The resulting mixture was stirred for additional 1 h at room temperature. The resulting mixture was filtered, the filter cake was washed with water (3×5 mL). The filtrate was concentrated under reduced pressure. This resulted in 2-[furo[3,2-b]pyridin-7-yl]-3-iodo-1H,5H,6H,7H-pyrrolo[3,2-c]pyridin-4-one (220.00 mg, 91.31%) as a yellow solid.

LC-MS: (M+H)⁺ found: 380.0.

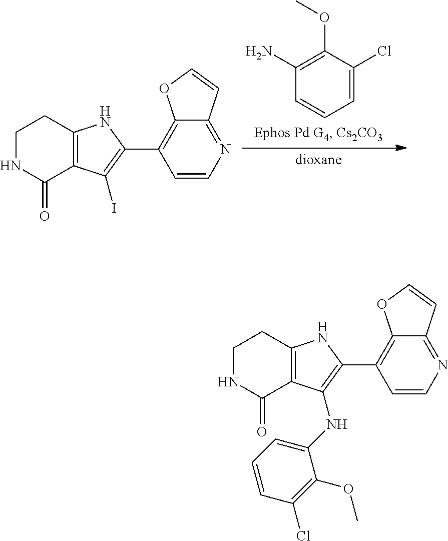

A mixture of 2-[furo[3,2-b]pyridin-7-yl]-3-iodo-1H,5H,6H,7H-pyrrolo[3,2-c]pyridin-4-one (100.00 mg, 0.26 mmol, 1.00 equiv) in dioxane (3 mL) under argon atmosphere. To the above mixture was added 3-chloro-2-methoxyaniline (42.00 mg, 0.26 mmol, 1.00 equiv), Ephos Pd G4 (48.00 mg, 0.05 mmol, 0.20 equiv) and Cs₂CO₃ (172.00 mg, 0.53 mmol, 2.00 equiv) under argon atmosphere. The resulting mixture was stirred for additional overnight at 50° C. The resulting mixture was washed with 3×50 mL of CH₂Cl₂/MeOH=10/1. The residue was purified by Prep-TLC (CH₂Cl₂/MeOH 10:1) to afford product. The crude product (80.00 mg) was purified by Prep-HPLC with the following conditions (Column: XBridge Prep C18 OBD Column, 30*100 mm, 5|Ìm; Mobile Phase A: Water (10 mmol/L NH₄HCO₃), Mobile Phase B: ACN: Flow rate: 60 mL/min; Gradient: 23% B to 35% B in 12 min, 35% B; Wave Length: 254/220 nm; RT1 (min): 11.65) to afford 3-[(3-chloro-2-methoxyphenyl)amino]-2-[furo[3,2-b]pyridin-7-yl]-1H,5H,6H,7H-pyrrolo[3,2-c]pyridin-4-one (50.60 mg, 46.69%) as a yellow solid.

LC-MS: (M+H)⁺ found: 409.0.

¹H NMR (300 MHz, DMSO-d₆) δ 11.64 (s, 1H), 8.39-8.27 (m, 2H), 7.58 (s, 1H), 7.30-7.18 (m, 2H), 7.12 (d, J=2.3 Hz, 1H), 6.70-6.57 (m, 2H), 6.14 (dd, J=7.8, 1.9 Hz, 1H), 3.91 (s, 3H), 3.44 (td, J1=6.8, 2.4 Hz, 2H), 2.93 (t, J=6.8 Hz, 2H).

### Example 144. 3-[(3-chloro-2-methoxyphenyl)amino]-2-[6-[(1S)-1-ethoxyethyl]-1,5-naphthyridin-4-yl]-1H,5H,6H,7H-pyrrolo[3,2-c]pyridin-4-one; trifluoroacetic acid (compound 754)

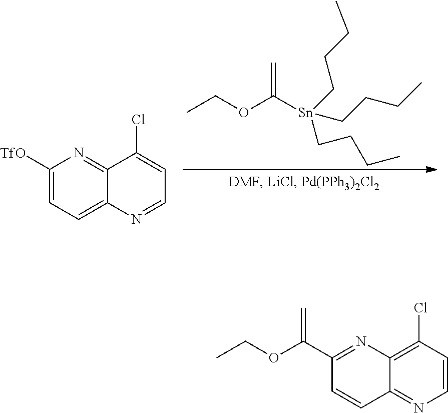 [SMILES: O=S(=O)(Oc1ccc2nccc(Cl)c2n1)C(F)(F)F ; C=[C](OCC)[Sn]([CH2]CCC)([CH2]CCC)[CH2]CCC ; C=C(OCC)c1ccc2nccc(Cl)c2n1]

Into a 40-mL vial, was placed 8-chloro-1,5-naphthyridin-2-yl trifluoromethanesulfonate (1.10 g, 3.52 mmol, 1.00 equiv), DMF (11.00 mL), LiCl (462.00 mg, 10.91 mmol, 3.10 equiv), Pd(PPh₃)₂Cl₂ (247.00 mg, 0.35 mmol, 0.10 equiv), tributyl(1-ethoxyethenyl)stannane (1.14 g, 3.17 mmol, 0.90 equiv). The resulting solution was stirred overnight at 60° C. The reaction was monitored by LCMS. Desired product could be detected by LCMS. The reaction was quenched with sat. KF (aq.) at room temperature. The resulting mixture was diluted with water (100 mL). The aqueous layer was extracted with EtOAc (3×100 mL). The resulting mixture was concentrated under reduced pressure. The residue was purified by Prep-TLC (CH₂Cl₂/MeOH 20:1) to afford 8-chloro-2-(1-ethoxyethenyl)-1,5-naphthyridine (600.00 mg, 71.21%) as a yellow solid.

LC-MS: (M+H)⁺ found: 235.0.

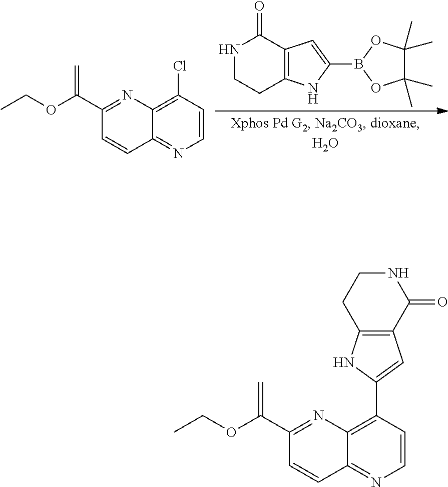 [SMILES: C=C(OCC)c1ccc2nccc(Cl)c2n1 ; CC1(C)OB(c2cc3c([nH]2)CCNC3=O)OC1(C)C ; C=C(OCC)c1ccc2nccc(-c3cc4c([nH]3)CCNC4=O)c2n1]

Into a 40-mL vial, was placed 8-chloro-2-(1-ethoxyethenyl)-1,5-naphthyridine (600.00 mg, 2.56 mmol, 1.00 equiv), dioxane (10 mL), H₂O (2 mL), 2-(4,4,5,5-tetramethyl-1,3,2-dioxaborolan-2-yl)-1H,5H,6H,7H-pyrrolo[3,2-c]pyridin-4-one (1.01 g, 3.84 mmol, 1.50 equiv), Na₂CO₃ (542.00 mg, 5.11 mmol, 2.00 equiv), XPhos palladium(II) biphenyl-2-amine chloride (402.00 mg, 0.51 mmol, 0.20 equiv). The resulting solution was stirred for 2 h at 60° C. The reaction was monitored by LCMS. The mixture was allowed to cool down to room temperature. The precipitated solids were collected by filtration and washed with water (3×10 mL) to afford 2-[6-(1-ethoxyethenyl)-1,5-naphthyridin-4-yl]-1H,5H,6H,7H-pyrrolo[3,2-c]pyridin-4-one (550.00 mg, 55.97%) as a yellow solid.

LC-MS: (M+H)⁺ found: 335.0.

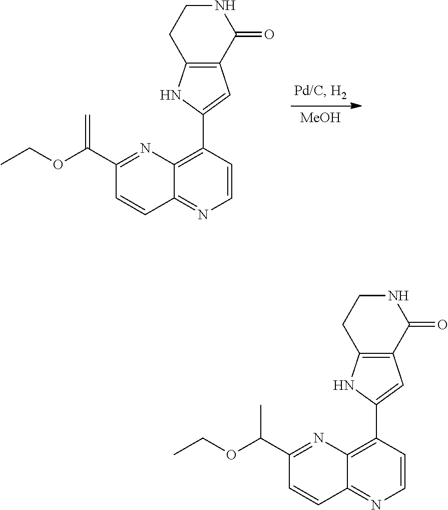 [SMILES: "MeOH" ; C=C(OCC)c1ccc2nccc(-c3cc4c([nH]3)CCNC4=O)c2n1 ; CCOC(C)c1ccc2nccc(-c3cc4c([nH]3)CCNC4=O)c2n1]

To a stirred mixture of 2-[6-(1-ethoxyethenyl)-1,5-naphthyridin-4-yl]-1H,5H,6H,7H-pyrrolo[3,2-c]pyridin-4-one (300.00 mg, 0.90 mmol, 1.00 equiv) in MeOH (5.00 mL) was added Pd/C (477.39 mg, 0.45 mmol, 0.50 equiv) in portions at room temperature under nitrogen atmosphere. The resulting mixture was stirred for 2 h at room temperature under hydrogen atmosphere. The reaction was monitored by LCMS. The resulting mixture was filtered, the filter cake was washed with MeOH (3×50 mL). The filtrate was concentrated under reduced pressure to afford 2-[6-(1-ethoxyethyl)-1,5-naphthyridin-4-yl]-1H,5H,6H,7H-pyrrolo[3,2-c]pyridin-4-one (220.00 mg, 62.69%) as a yellow solid.

LC-MS: (M+H)⁺ found: 337.0.

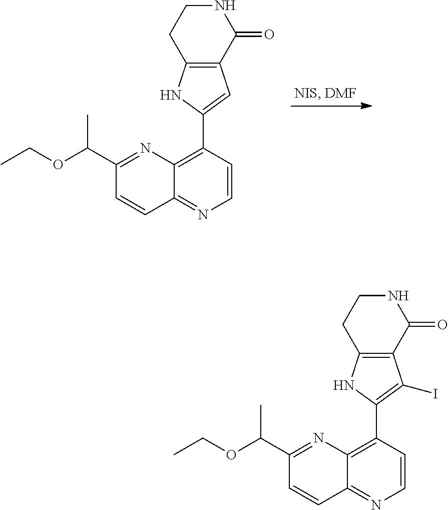 [SMILES: CCOC(C)c1ccc2nccc(-c3cc4c([nH]3)CCNC4=O)c2n1 ; CCOC(C)c1ccc2nccc(-c3[nH]c4c(c3I)C(=O)NCC4)c2n1]

Into a 40-mL vial, was placed 2-[6-(1-ethoxyethyl)-1,5-naphthyridin-4-yl]-1H,5H,6H,7H-pyrrolo[3,2-c]pyridin-4-one (360.00 mg, 1.07 mmol, 1.00 equiv), DMF (10 mL), NIS (289.00 mg, 1.28 mmol, 1.20 equiv). The resulting solution was stirred for 1 h at room temperature. The reaction was monitored by LCMS. The reaction was quenched by the addition of sat. Na₂SO₃ (aq.) (15 mL) at 0° C. The precipitated solids were collected by filtration and washed with water (3×50 mL) to afford 2-[6-(1-ethoxyethyl)-1,5-naphthyridin-4-yl]-3-iodo-1H,5H,6H,7H-pyrrolo[3,2-c]pyridin-4-one (400.00 mg, 78.43%) as a yellow solid.

LC-MS: (M+H)⁺ found: 463.0.

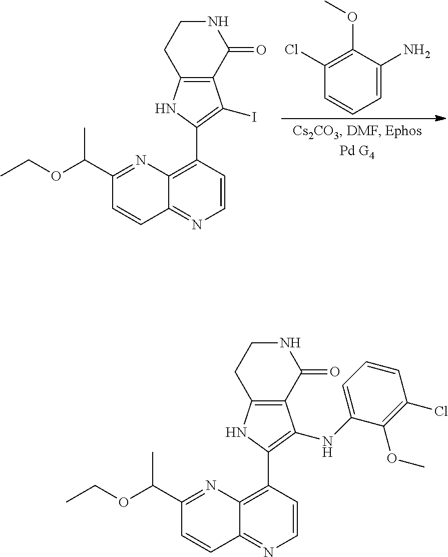

Into a 40-mL vial, was placed 2-[6-(1-ethoxyethyl)-1,5-naphthyridin-4-yl]-3-iodo-1H,5H,6H,7H-pyrrolo[3,2-c]pyridin-4-one (400.00 mg, 0.87 mmol, 1.00 equiv), DMF (9 mL), 3-chloro-2-methoxyaniline (136.00 mg, 0.87 mmol, 1.00 equiv), Ephos Pd G4 (79.00 mg, 0.09 mmol, 0.10 equiv), Cs₂CO₃ (564.00 mg, 1.73 mmol, 2.00 equiv). The resulting solution was stirred for 3 h at 50° C. The reaction was monitored by LCMS. The resulting mixture was diluted with water (100 mL). The aqueous layer was extracted with EtOAc (3×100 mL). The resulting mixture was concentrated under reduced pressure. The residue was purified by Prep-TLC (CH₂Cl₂/MeOH 15:1) to afford 3-[(3-chloro-2-methoxyphenyl)amino]-2-[6-(1-ethoxyethyl)-1,5-naphthyridin-4-yl]-1H,5H,6H,7H-pyrrolo[3,2-c]pyridin-4-one (300.00 mg) as a red solid. The crude product (300.00 mg) was purified by Prep-HPLC with the following conditions (Column: XBridge Prep OBD C18 Column, 30*150 mm, 5|Ìm; Mobile Phase A: Water (0.05% TFA), Mobile Phase B: ACN; Flow rate: 60 mL/min; Gradient: 21% B to 51% B in 7 min; Wave Length: 254 nm; RT1 (min): 6.5) to afford 3-[(3-chloro-2-methoxyphenyl)amino]-2-[6-(1-ethoxyethyl)-1,5-naphthyridin-4-yl]-1H,5H,6H,7H-pyrrolo[3,2-c]pyridin-4-one (120.00 mg, 27.91%) as a red solid.

LC-MS: (M+H)⁺ found: 492.0.

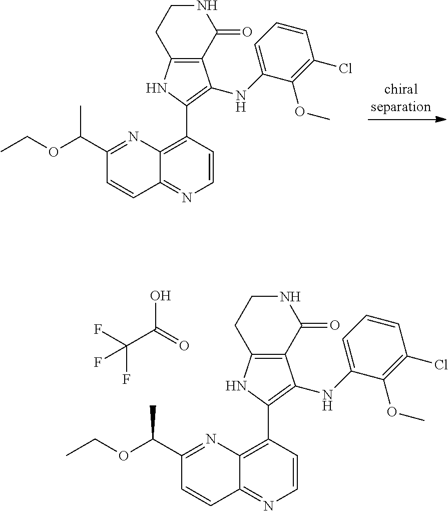

The crude product (120.00 mg) was purified by Prep-CHIRAL-HPLC with the following conditions (Column: CHIRALPAK IH, 3*25 cm, 5 μm; Mobile Phase A: Hex (0.5% 2M NH3-MeOH)-HPLC, Mobile Phase B: EtOH-HPLC; Flow rate: 20 mL/min; Gradient: 10% B to 10% B in 19.5 min; Wave Length: 220/254 nm; RT1 (min): 20.524; RT2 (min): 24.81; Sample Solvent: EtOH-HPLC; Injection Volume: 1.5 mL; Number Of Runs: 17) to afford 3-[(3-chloro-2-methoxyphenyl)amino]-2-[6-[(1S)-1-ethoxyethyl]-1,5-naphthyridin-4-yl]-1H,5H,6H,7H-pyrrolo[3,2-c]pyridin-4-one; trifluoroacetic acid (37.80 mg, 25.29%) as a yellow solid.

LC-MS: (M+H)⁺ found: 492.0.

¹H NMR (300 MHz, DMSO-d₆) δ 12.84 (s, 1H), 8.73 (d, J=4.9 Hz, 1H), 8.46 (d, J=8.8 Hz, 1H), 7.90 (d, J=8.6 Hz, 2H), 7.52 (d, J=4.8 Hz, 1H), 7.28 (t, J=2.5 Hz, 1H), 6.81-6.66 (m, 2H), 6.20 (dd, J=7.7, 1.9 Hz, 1H), 4.96 (q, J=6.5 Hz, 1H), 3.92 (s, 3H), 3.60 (dq, J=9.2, 7.0 Hz, 1H), 3.53-3.36 (m, 3H), 3.01 (t, J=6.8 Hz, 2H), 1.54 (d, J=6.5 Hz, 3H), 1.21 (t, J=7.0 Hz, 3H).

### Example 145. 3-[(3-chloro-2-methoxyphenyl)amino]-2-[6-[(1R)-1-ethoxyethyl]-1,5-naphthyridin-4-yl]-1H,5H,6H,7H-pyrrolo[3,2-c]pyridin-4-one; trifluoroacetic acid (compound 753)

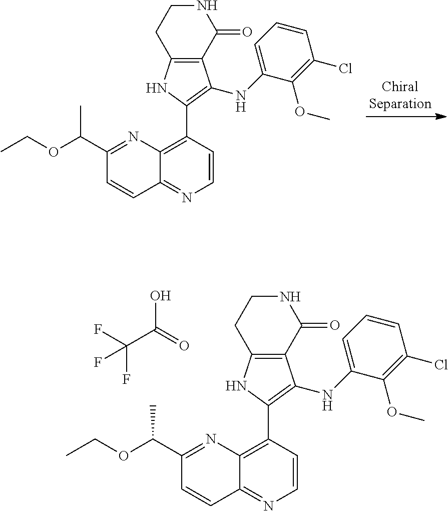

The crude product (120.00 mg) was purified by Prep-CHIRAL-HPLC with the following conditions (Column: CHIRALPAK IH, 3*25 cm, 5 μm; Mobile Phase A: Hex (0.5% 2M NH₃-MeOH)-HPLC, Mobile Phase B: EtOH-HPLC; Flow rate: 20 mL/min; Gradient: 10% B to 10% B in 19.5 min; Wave Length: 220/254 nm: RT1 (min): 20.524; RT2 (min): 24.81; Sample Solvent: EtOH-HPLC; Injection Volume: 1.5 mL; Number Of Runs: 17) to afford 3-[(3-chloro-2-methoxyphenyl)amino]-2-[6-[(1R)-1-ethoxyethyl]-1,5-naphthyridin-4-yl]-1H,5H,6H,7H-pyrrolo[3,2-c]pyridin-4-one; trifluoroacetic acid (41.70 mg, 28.04%) as a red solid.

LC-MS: (M+H)⁺ found: 492.0.

¹H NMR (300 MHz, DMSO-d₆) δ 12.85 (s, 1H), 8.75 (d, J=5.0 Hz, 1H), 8.47 (d, J=8.8 Hz, 1H), 8.01 (s, 1H), 7.94 (d, J=8.8 Hz, 1H), 7.50 (d, J=5.0 Hz, 1H), 7.29 (d, J=16.8 Hz, 1H), 6.79 (dd, J=8.1, 1.8 Hz, 1H), 6.73 (t, J=8.0 Hz, 1H), 6.21 (dd, J=7.9, 1.7 Hz, 1H), 4.97 (q, J=6.5 Hz, 1H), 3.92 (s, 3H), 3.61 (dq, J=9.1, 6.9 Hz, 1H), 3.47 (dtd, J=13.7, 6.9, 2.3 Hz, 3H), 3.02 (t, J=6.8 Hz, 2H), 1.54 (d, J=6.5 Hz, 3H), 1.21 (t, J=7.0 Hz, 3H).

### Example 146. 3-[(3-chloro-2-methoxyphenyl)amino]-2-(6-cyclopropyl-1,5-naphthyridin-4-yl)-1H,5H,6H,7H-pyrrolo[3,2-c]pyridin-4-one (compound 752)

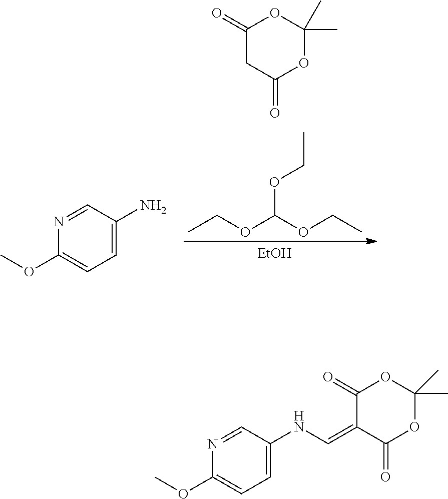

Into a 500-mL round-bottom flask, was placed 5-amino-2-methoxypyridine (10.00 g, 80.55 mmol, 1.00 equiv), ethyl alcohol (100 mL), meldrum (11.61 g, 80.55 mmol, 1.00 equiv), triethyl orthoformate (11.94 g, 80.55 mmol, 1.00 equiv). The resulting solution was stirred for 4 h at 85° C. The reaction was monitored by LCMS. The mixture was allowed to cool down to room temperature. The precipitated solids were collected by filtration and washed with EtOH (3×30 mL) to afford 5-[[(6-methoxypyridin-3-yl)amino]methylidene]-2,2-dimethyl-1,3-dioxane-4,6-dione (19.00 g, 84.77%) as an off-white solid.

LC-MS: (M+H)⁺ found: 279.0.

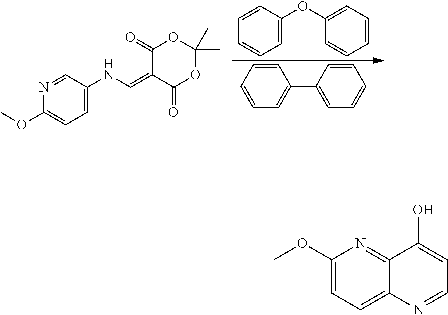

Into a 500-mL 3-necked round-bottom flask, was placed diphenyl-ether (110.25 mL), diphenyl (39.75 mL). This was followed by the addition of 5-[[(6-methoxypyridin-3-yl)amino]methylidene]-2,2-dimethyl-1,3-dioxane-4,6-dione (12.00 g, 43.13 mmol, 1.00 equiv) at 250° C. The resulting solution was stirred for 15 min at 250° C. Desired product could be detected by LCMS. The mixture was allowed to cool down to room temperature. The resulting mixture was diluted with diethyl ether (100 mL). The precipitated solids were collected by filtration and washed with hexane (2×100 mL) to afford 6-methoxy-1,5-naphthyridin-4-ol (6.00 g, 72.66%) as a Brown yellow solid.

LC-MS: (M+H)⁺ found: 177.0.

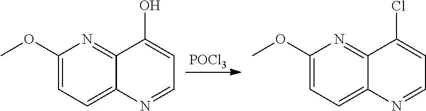

Into a 40-mL vial, was placed 6-methoxy-1,5-naphthyridin-4-ol (5.00 g, 28.38 mmol, 1.00 equiv), phosphorus oxychloride (50 mL). The resulting solution was stirred for 1 h at 100° C. The reaction was monitored by LCMS. The mixture was allowed to cool down to room temperature. The resulting mixture was concentrated under reduced pressure. The mixture was neutralized to pH 7 with NaOH. The precipitated solids were collected by filtration and washed with water (2×50 mL) to afford 8-chloro-2-methoxy-1,5-naphthyridine (5.00 g, 84.19%) as an off-white solid.

LC-MS: (M+H)⁺ found: 195.0.

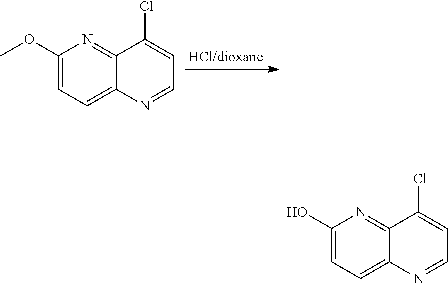

Into a 100-mL round-bottom flask, was placed 8-chloro-2-methoxy-1,5-naphthyridine (2.00 g, 10.28 mmol, 1.00 equiv), HCl (gas) in 1,4-dioxane (25 mL). The resulting solution was stirred for 24 h at 100° C. The reaction was monitored by LCMS. The mixture was allowed to cool down to room temperature. The precipitated solids were collected by filtration and washed with DCM (2×3 mL) to afford 8-chloro-1,5-naphthyridin-2-ol (1.40 g, 73.93%) as a Brown yellow solid.

LC-MS: (M+H)⁺ found: 181.0.

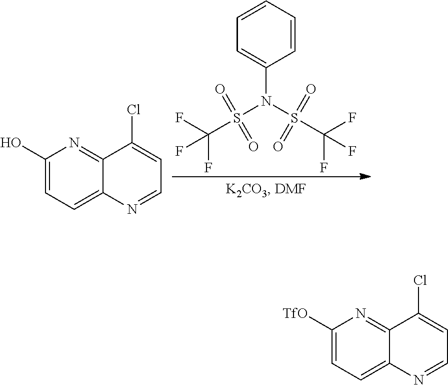

Into a 50-mL round-bottom flask, was placed 8-chloro-1,5-naphthyridin-2-ol (1.40 g, 7.75 mmol, 1.00 equiv), DMF (20 mL), K₂CO₃ (3.75 g, 27.13 mmol, 3.50 equiv), 1,1,1-trifluoro-N-phenyl-N-trifluoro methanesulfonylmethanesulfonamide (2.91 g, 8.14 mmol, 1.05 equiv). The resulting solution was stirred for 4 h at room temperature. The reaction was monitored by LCMS. The resulting mixture was filtered, the filter cake was washed with DMF (2×3 mL). The resulting mixture was diluted with water (50 mL). The aqueous layer was extracted with EtOEt (3×50 mL). The resulting mixture was concentrated under reduced pressure to afford 8-chloro-1,5-naphthyridin-2-yl trifluoromethanesulfonate (1.60 g, 63.37%) as a Brown yellow solid.

LC-MS: (M+H)⁺ found: 313.0.

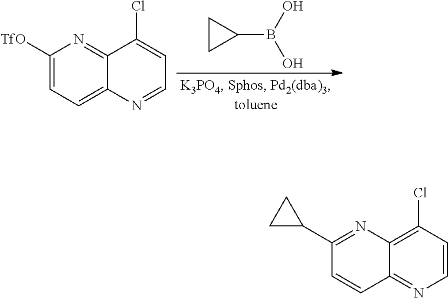

Into a 40-mL vial, was placed 8-chloro-1,5-naphthyridin-2-yl trifluoromethanesulfonate (500.00 mg, 1.60 mmol, 1.00 equiv), toluene (10 mL), cyclopropylboronic acid (144.00 mg, 1.68 mmol, 1.05 equiv), K₃PO₄ (1.02 g, 4.80 mmol, 3.00 equiv), Sphos (328.00 mg, 0.80 mmol, 0.50 equiv), Pd₂(dba)₃ (293.00 mg, 0.32 mmol, 0.20 equiv). The resulting solution was stirred overnight at 40° C. The reaction was monitored by LCMS. The resulting mixture was concentrated under reduced pressure to afford 8-chloro-2-cyclopropyl-1,5-naphthyridine (300.00 mg, 49.50%) as a yellow solid. The residue was used in the next step directly without further purification.

LC-MS: (M+H)⁺ found: 205.0.

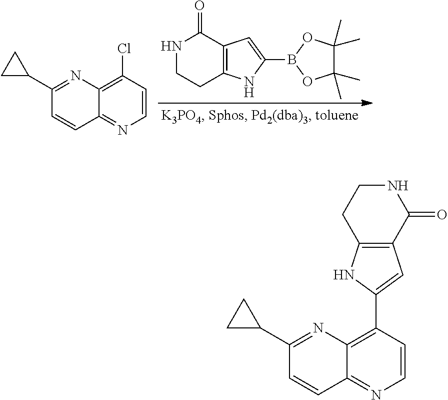

Into a 40-mL vial, was placed 8-chloro-2-cyclopropyl-1,5-naphthyridine (300.00 mg, 1.47 mmol, 1.00 equiv), toluene (10 mL), 2-(4,4,5,5-tetramethyl-1,3,2-dioxaborolan-2-yl)-1H,5H,6H,7H-pyrrolo[3,2-c]pyridine-4-one (576.00 mg, 2.20 mmol, 1.50 equiv), K₃PO₄ (933.00 mg, 4.40 mmol, 3.00 equiv), Sphos (301.00 mg, 0.73 mmol, 0.50 equiv), Pd₂(dba)₃ (268.00 mg, 0.29 mmol, 0.20 equiv). The resulting solution was stirred for 5 h at 60° C. The reaction was monitored by LCMS. The resulting mixture was concentrated under reduced pressure. The resulting mixture was diluted with MeOH (3 mL). The residue was purified by reverse flash chromatography with the following conditions (column, C18 silica gel; mobile phase, MeOH in water, 10% to 50% gradient in 10 min; detector, UV 254 nm) to afford 2-(6-cyclopropyl-1,5-naphthyridin-4-yl)-1H,5H,6H,7H-pyrrolo[3,2-c]pyridin-4-one (130.00 mg, 25.35%) as a yellow solid.

LC-MS: (M+H)⁺ found: 305.0.

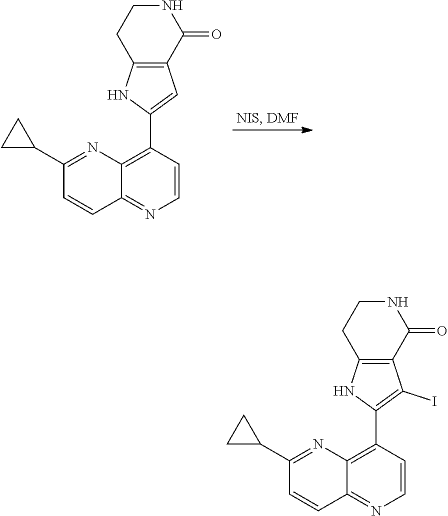 [SMILES: O=C1NCCc2[nH]c(-c3ccnc4ccc(C5CC5)nc34)cc21 ; O=C1NCCc2[nH]c(-c3ccnc4ccc(C5CC5)nc34)c(I)c21]

Into a 20-mL vial, was placed 2-(6-cyclopropyl-1,5-naphthyridin-4-yl)-1H,5H,6H,7H-pyrrolo[3,2-c]pyridin-4-one (120.00 mg, 0.39 mmol, 1.00 equiv), DMF (5 mL), NIS (106.00 mg, 0.47 mmol, 1.20 equiv). The resulting solution was stirred for 3 h at room temperature. The reaction was monitored by LCMS. The reaction was quenched with sat. Na₂SO₃ (aq.) at 0° C. The precipitated solids were collected by filtration and washed with water (3×6 mL) to afford 2-(6-cyclopropyl-1,5-naphthyridin-4-yl)-3-iodo-1H,5H,6H,7H-pyrrolo[3,2-c]pyridin-4-one (90.00 mg, 36.61%) as a yellow solid.

LC-MS: (M+H)⁺ found: 431.0.

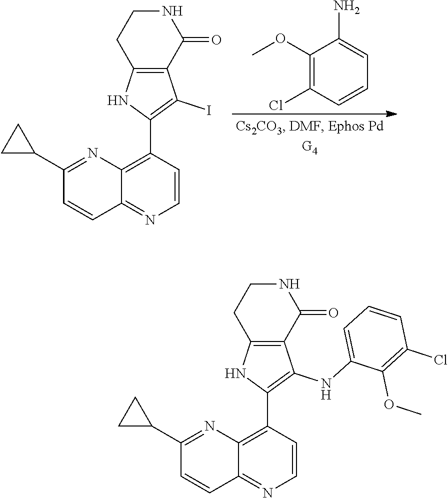

Into a 20-mL vial, was placed 2-(6-cyclopropyl-1,5-naphthyridin-4-yl)-3-iodo-1H,5H,6H,7H-pyrrolo[3,2-c]pyridin-4-one (90.00 mg, 0.21 mmol, 1.00 equiv), DMF (4 mL), 3-chloro-2-methoxyaniline (33.00 mg, 0.21 mmol, 1.00 equiv), Ephos Pd G4 (38.00 mg, 0.04 mmol, 0.20 equiv), Cs₂CO₃ (136.00 mg, 0.42 mmol, 2.00 equiv). The resulting solution was stirred for 3 h at 50° C. The reaction was monitored by LCMS. The resulting mixture was diluted with water (50 mL). The aqueous layer was extracted with EtOAc (3×50 mL). The resulting mixture was concentrated under reduced pressure. The residue was purified by Prep-TLC (CH₂Cl₂/MeOH 15:1) to afford 3-[(3-chloro-2-methoxyphenyl)amino]-2-(6-cyclopropyl-1,5-naphthyridin-4-yl)-1H,5H,6H,7H-pyrrolo[3,2-c]pyridin-4-one (45.00 mg) as a yellow solid. The crude product (45.00 mg) was purified by Prep-HPLC with the following conditions (Column: XBridge Prep C18 OBD Column, 30*100 mm, 5|Ìm; Mobile Phase A: Water (10 mmol/L NH₄HCO₃), Mobile Phase B: ACN; Flow rate: 60 mL/min; Gradient: 36% B to 66% B in 7 min, 66% B; Wave Length: 254/220 nm; RT1 (min): 6.32) to afford 3-[(3-chloro-2-methoxyphenyl)amino]-2-(6-cyclopropyl-1,5-naphthyridin-4-yl)-1H,5H,6H,7H-pyrrolo[3,2-c]pyridin-4-one (20.80 mg, 21.55%) as a yellow solid.

LC-MS: (M+H)⁺ found: 460.0.

¹H NMR (300 MHz, DMSO-d₆) δ 12.62 (s, 1H), 8.64 (d, J=4.8 Hz, 1H), 8.29 (d, J=8.8 Hz, 1H), 7.84 (s, 1H), 7.68 (d, J=8.8 Hz, 1H), 7.48 (d, J=4.8 Hz, 1H), 7.24 (d, J=2.6 Hz, 1H), 6.78-6.64 (m, 2H), 6.19 (dd, J=7.3, 2.3 Hz, 1H), 3.88 (s, 3H), 3.47 (td, J=6.7, 2.5 Hz, 2H), 2.98 (t, J=6.7 Hz, 2H), 2.64-2.52 (m, 1H), 1.32-1.13 (m, 4H).

### Example 147. 8-(3-((3-chloro-2-methoxyphenyl)amino)-4-oxo-4,5,6,7-tetrahydro-1H-pyrrolo[3,2-c]pyridin-2-yl)-2-methoxypyrido[2,3-b]pyrazin-3 (4H)-one (compound 729)

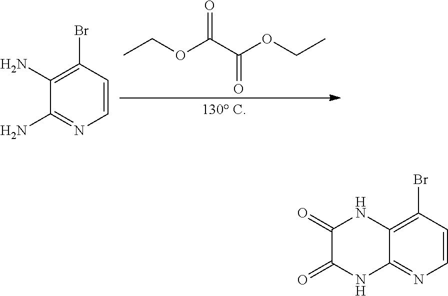

4-bromopyridine-2,3-diamine (3.00 g, 16.04 mmol, 1.00 equiv) was dissolved in diethyl oxalate (10 mL). The resulting mixture was stirred for 16 h at 130° C. The reaction was monitored by TLC and LCMS. The precipitated solids were collected by filtration and washed with petroleum ether (3×20 mL). This resulted in 8-bromo-1,4-dihydropyrido[2,3-b]pyrazine-2,3-dione (3.80 g, 77.69%) as a yellow solid.

LC-MS: (M+H)⁺ found: 242.0.

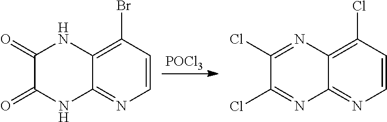

8-bromo-1,4-dihydropyrido[2,3-b]pyrazine-2,3-dione (3.00 g, 0.20 mmol, 1.00 equiv) was dissolved in POCl₃ (20.0 mL). The resulting mixture was stirred for overnight at 130° C. and cooled to room temperature. The mixture was poured into the ice water (100 mL). The precipitated solids were collected by filtration and washed with petroleum ether (3×20 mL) to afford 2,3,8-trichloropyrido[2,3-b]pyrazine (1.50 g, 51.72%) as a brown solid.

LC-MS: (M+H)⁺ found: 234.0.

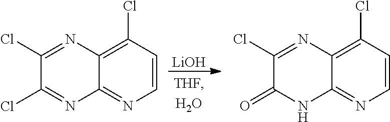

2,3,8-trichloropyrido[2,3-b]pyrazine (1.50 g, 6.44 mmol, 1.00 equiv) was dissolved in THF (20.0 mL) and H₂O (8 mL). Then LiOH (309.00 mg, 12.88 mmol, 2.00 equiv) was added. The resulting mixture was stirred for 3 h at 50° C. The precipitated solids were collected by filtration and washed with petroleum ether (3×20 mL) to afford 2,8-dichloropyrido[2,3-b]pyrazin-3 (4H)-one (1.10 g, 79.71%) as a yellow solid.

LC-MS: (M+H)⁺ found: 216.0.

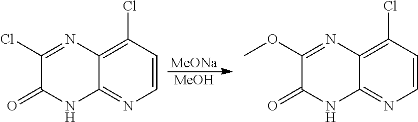

2,8-dichloropyrido[2,3-b]pyrazin-3 (4H)-one (1.00 g, 4.65 mmol, 1.00 equiv) was dissolved in MeOH (10.0 mL). Then NaOMe (502.20 mg, 9.30 mmol, 2.00 equiv) was added. The resulting mixture was stirred for overnight at 50° C. and cooled to room temperature. The mixture was poured into NH₄Cl aq. (30 mL). The precipitated solids were collected by filtration and washed with petroleum ether (3×10 mL). This resulted in 8-chloro-2-methoxypyrido[2,3-b]pyrazin-3 (4H)-one (500.00 mg, 51.0%) as a brown solid.

LC-MS: (M+H)⁺ found: 212.0.

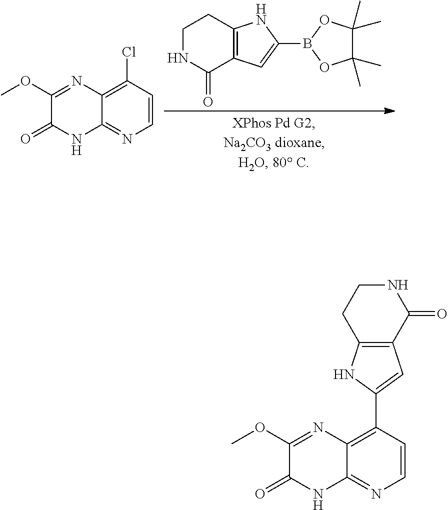 [SMILES: COc1nc2c(Cl)ccnc2[nH]c1=O ; CC1(C)OB(c2cc3c([nH]2)CCNC3=O)OC1(C)C ; COc1nc2c(-c3cc4c([nH]3)CCNC4=O)ccnc2[nH]c1=O]

8-chloro-2-methoxypyrido[2,3-b]pyrazin-3 (4H)-one (250.00 mg, 1.18 mmol, 1.00 equiv) was dissolved in dioxane (5.0 mL) and H₂O (0.2 mL). Then 2-(4,4,5,5-tetramethyl-1,3,2-dioxaborolan-2-yl)-1,5,6,7-tetrahydro-4H-pyrrolo[3,2-c]pyridin-4-one (463.70 mg, 1.77 mmol, 1.50 equiv), XPhos Pd G 2 (94.30 mg, 0.12 mmol, 0.10 equiv) and Na₂CO₃ (375.20 mg, 3.54 mmol, 3.00 equiv) were added. The resulting mixture was stirred for 2 h at 80° C. under N₂ and cooled to room temperature. The precipitated solids were collected by filtration and washed with dioxane (3×5 mL) to afford 2-methoxy-8-(4-oxo-4,5,6,7-tetrahydro-1H-pyrrolo[3,2-c]pyridin-2-yl)pyrido[2,3-b]pyrazin-3 (4H)-one (260.00 mg, 70.7%) as a yellow solid.

LC-MS: (M+H)⁺ found: 312.0.

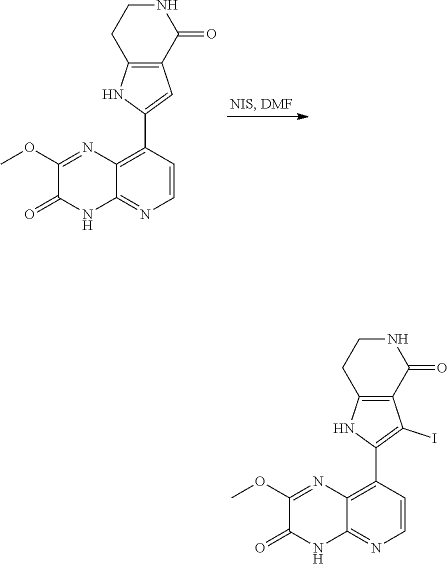 [SMILES: COc1nc2c(-c3cc4c([nH]3)CCNC4=O)ccnc2[nH]c1=O ; COc1nc2c(-c3[nH]c4c(c3I)C(=O)NCC4)ccnc2[nH]c1=O]

2-methoxy-8-(4-oxo-4,5,6,7-tetrahydro-1H-pyrrolo[3,2-c]pyridin-2-yl)pyrido[2,3-b]pyrazin-3 (4H)-one (260.00 mg, 0.84 mmol, 1.00 equiv) was dissolved in DMF (3.0 mL). Then NIS (378.00 mg, 1.68 mmol, 2.00 equiv) was added. The resulting mixture was stirred for overnight at RT. The mixture was poured into Na₂SO₃ aq. (0.5 mL). The precipitated solids were collected by filtration and washed with petroleum ether (3×3 mL) to afford 8-(3-iodo-4-oxo-4,5,6,7-tetrahydro-1H-pyrrolo[3,2-c]pyridin-2-yl)-2-methoxypyrido [2,3-b]pyrazin-3 (4H)-one (160.00 mg, 43.8%) as a yellow solid.

LC-MS: (M+H)⁺ found: 438.0.

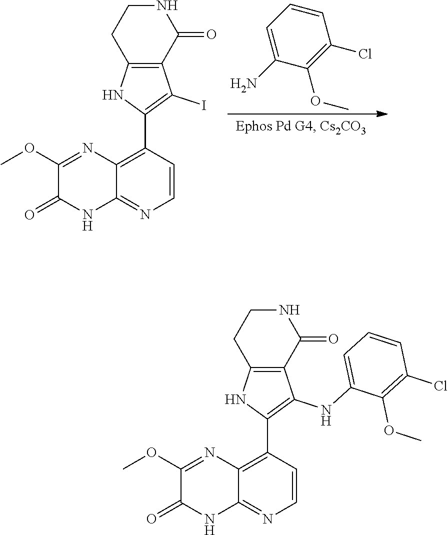

8-(3-iodo-4-oxo-4,5,6,7-tetrahydro-1H-pyrrolo[3,2-c]pyridin-2-yl)-2-methoxypyrido[2,3-b]pyrazin-3 (4H)-one (100.00 mg, 0.23 mmol, 1.00 equiv) was dissolved in DMF (2 mL). Then 3-chloro-2-methoxyaniline (35.90 mg, 0.23 mmol, 1.00 equiv), EPhos Pd G 4 (21.01 mg, 0.02 mmol, 0.10 equiv) and Cs₂CO₃ (149.05 mg, 0.46 mmol, 2.00 equiv) were added. The resulting mixture was stirred for 3 h at 50° C. under N2 and cooled to room temperature. The crude product was purified by Prep-HPLC with the following conditions ((2 #SHIMADZU HPLC-01): Column, XBridge Prep OBD C18 Column, 30*150 mm, 5 μm; mobile phase, Water (10 mmol/L NH₄HCO₃) and CAN (18% Phase B up to 48% in 7 min); Detector, UV 254/210 nm) to afford 8-(3-((3-chloro-2-methoxyphenyl)amino)-4-oxo-4,5,6,7-tetrahydro-1H-pyrrolo[3,2-c]pyridin-2-yl)-2-methoxypyrido[2,3-b]pyrazin-3 (4H)-one (24.00 mg, 22.47%) as a yellow solid.

LC-MS: (M+H)⁺ found: 467.0.

¹H NMR (300 MHz, DMSO-d₆): δ 12.44 (s, 1H), 11.62 (s, 1H), 8.12 (d, J=5.1 Hz, 1H), 7.68 (s, 1H), 7.21 (d, J=5.1 Hz, 1H), 7.16 (s, 1H), 6.69-6.64 (m, 2H), 6.20 (dd, J=5.7, 3.9 Hz, 1H), 4.13 (s, 3H), 3.87 (s, 3H), 3.45-3.43 (m, 2H), 2.94-2.90 (m, 2H).

### Example 148. 3-[(3-chloro-2-methoxyphenyl)amino]-2-(6-[[1-(trifluoromethyl)cyclopropyl]methoxy]-1,5-naphthyridin-4-yl)-1H,5H,6H,7H-pyrrolo[3,2-c]pyridin-4-one (compound 758)

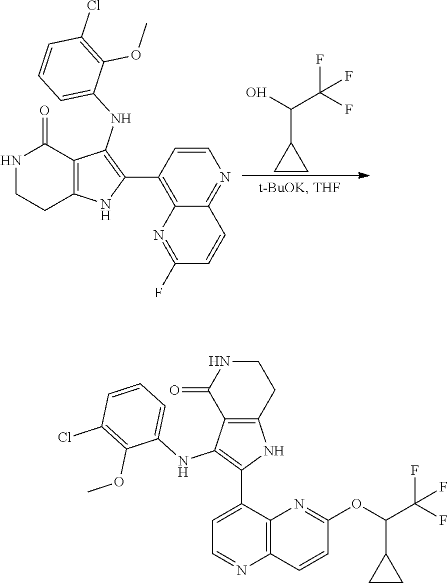 [SMILES: COc1c(Cl)cccc1Nc1c(-c2ccnc3ccc(F)nc23)[nH]c2c1C(=O)NCC2 ; OC(C1CC1)C(F)(F)F ; COc1c(Cl)cccc1Nc1c(-c2ccnc3ccc(OC(C4CC4)C(F)(F)F)nc23)[nH]c2c1C(=O)NCC2]

To a stirred solution of 3-[(3-chloro-2-methoxyphenyl) amino]-2-(6-fluoro-1,5-naphthyridin-4-yl)-1H,5H,6H,7H-pyrrolo[3,2-c]pyridin-4-one (45.00 mg, 0.10 mmol, 1.00 equiv) and [1-(trifluoromethyl) cyclopropyl]methanol (72.00 mg, 0.51 mmol, 5.00 equiv) in THF (1 mL) was added t-BuOK (35.00 mg, 0.31 mmol, 3.00 equiv) dropwise at 0° C. The resulting mixture was stirred for 1 h at room temperature. The reaction was monitored by LCMS. The crude product was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 41% B to 63% B in 8 min, 63% B; Wave Length: 254/220 nm; RT1 (min): 8) to afford 3-[(3-chloro-2-methoxyphenyl)amino]-2-(6-[[1-(trifluoromethyl)cyclopropyl]methoxy]-1,5-naphthyridin-4-yl)-1H,5H,6H,7H-pyrrolo[3,2-c]pyridin-4-one (14.70 mg, 25.15%) as a yellow solid.

LC-MS: (M+H)⁺ found: 558.0.

¹H NMR (300 MHz, DMSO-d₆) δ 11.88 (s, 1H), 8.58 (d, J=4.8 Hz, 1H), 8.31 (d, J=9.0 Hz, 1H), 7.75 (s, 1H), 7.52 (d, J=4.8 Hz, 1H), 7.36 (d, J=9.0 Hz, 1H), 7.21 (s, 1H), 6.69-6.58 (m, 2H), 6.15-6.12 (m, 1H), 4.78 (s, 2H) 3.88 (s, 3H), 3.48-3.46 (m, 2H), 2.92 (t, J=6.9 Hz, 2H), 1.17-1.10 (m, 4H).

### Example 149. 3-[(3-chloro-2-methoxyphenyl)amino]-2-[6-[(2R)-oxetan-2-ylmethoxy]-1,5-naphthyridin-4-yl]-1H,5H,6H,7H-pyrrolo[3,2-c]pyridin-4-one (compound 734)

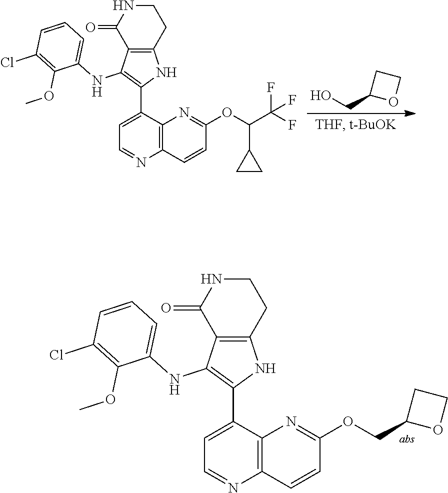

To a stirred solution of 3-[(3-chloro-2-methoxyphenyl)amino]-2-(1,5-naphthyridin-4-yl)-1H,5H,6H,7H-pyrrolo[3,2-c]pyridin-4-one (45.00 mg, 0.11 mmol, 1.00 equiv) and (2R)-oxetan-2-ylmethanol (9.44 mg, 0.11 mmol, 1.00 equiv) in THF (3 mL) was added t-BuOK (36.08 mg, 0.32 mmol, 3.00 equiv) in portions at rt under Ar atmosphere. The resulting mixture was stirred for 1 h at 50° C. under Ar atmosphere. Desired product could be detected by LCMS. The resulting mixture was concentrated under reduced pressure and dissolved in DMF. The residue was purified by silica gel column with the following conditions (column, silica gel; mobile phase, DCM in MeOH, 0% to 10% gradient in 10 min; detector, UV 254 nm) to afford crude product (45.00 mg). The crude product was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 m/min; Gradient: 26% B to 56% B in 7 min, 56% B; Wave Length: 254 nm; RT1 (min): 6.5) to afford 3-[(3-chloro-2-methoxyphenyl)amino]-2-[6-[(2R)-oxetan-2-ylmethoxy]-1,5-naphthyridin-4-yl]-1H,5H,6H,7H-pyrrolo[3,2-c]pyridin-4-one (9.20 mg, 16.73%) as a yellow solid.

LC-MS: (M+H)⁺ found: 506.2.

¹H NMR (400 MHz, DMSO-d₆) δ 12.06 (s, JH), 8.58 (d, J=4.8 Hz, 1H), 8.32 (d, J=9.1 Hz, 1H), 7.78 (s, 1H), 7.51 (d, J=4.9 Hz, 1H), 7.41 (d, J=9.1 Hz, 1H), 7.20 (t, J=2.4 Hz, 1H), 6.73-6.62 (m, 2H), 6.18 (dd, J=7.9, 1.8 Hz, 1H), 5.20 (q, J=6.6, 5.3 Hz, 1H), 4.94-4.25 (m, 4H), 3.90 (s, 3H), 3.32 (s, 2H), 2.95 (d, J=1.7 Hz, 2H), 2.51 (p, J=1.9 Hz, 2H).

### Example 150. 3-[(3-chloro-2-methoxyphenyl)amino]-2-(6-isopropoxy-1,5-naphthyridin-4-yl)-1H,5H,6H,7H-pyrrolo[3,2-c]pyridin-4-one (compound 762)

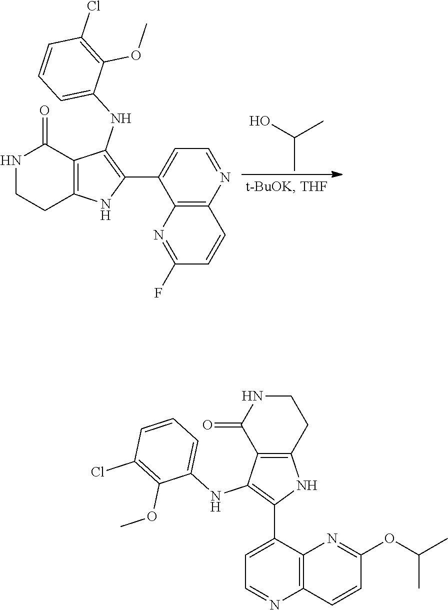

To a stirred solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (40.00 mg, 0.09 mmol, 1.00 equiv) and Cs₂CO₃ (89.29 mg, 0.27 mmol, 3.00 equiv) in isopropyl alcohol (0.50 mL) at 0° C. under nitrogen atmosphere. The resulting mixture was stirred overnight at room temperature under nitrogen atmosphere. The resulting mixture was concentrated under vacuum. The crude product (60.00 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 47% B to 72% B in 9 min, 72% B; Wave Length: 254/220 nm; RT1 (min): 8.85) to afford 3-[(3-chloro-2-methoxyphenyl)amino]-2-(6-isopropoxy-1,5-naphthyridin-4-yl)-1H,5H,6H,7H-pyrrolo[3,2-c]pyridin-4-one (10.10 mg, 23.13%) as a yellow solid.

LC-MS: (M+H)⁺ found: 478.0.

¹H NMR (400 MHz, DMSO-d₆) δ 12.18 (s, 1H), 8.56 (d, J=4.9 Hz, 1H), 8.27 (d, J=9.1 Hz, 1H), 7.71 (s, 1H), 7.50 (d, J=4.9 Hz, 1H), 7.28-7.23 (m, 2H), 6.71-6.67 (t, J=7.9 Hz, 2H), 6.18 (dd, J=7.7, 1.9 Hz, 1H), 5.45-5.51 (m, J=6.2 Hz, 1H), 3.89 (s, 3H), 3.47 (td, J=6.9, 2.5 Hz, 2H), 2.95 (t, J=6.8 Hz, 2H), 1.47 (d, J=6.2 Hz, 6H).

### Example 151. 3-[(3-chloro-2-methoxyphenyl)amino]-2-(6-ethoxy-1,5-naphthyridin-4-yl)-1H,5H,6H,7H-pyrrolo[3,2-c]pyridin-4-one (compound 761)

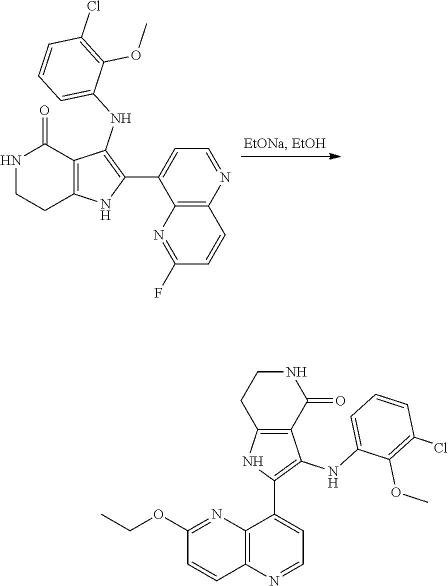 [SMILES: COc1c(Cl)cccc1Nc1c(-c2ccnc3ccc(F)nc23)[nH]c2c1C(=O)NCC2 ; CCOc1ccc2nccc(-c3[nH]c4c(c3Nc3cccc(Cl)c3OC)C(=O)NCC4)c2n1]

A mixture of 3-[(3-chloro-2-methoxyphenyl)amino]-2-(6-fluoro-1,5-naphthyridin-4-yl)-1H,5H,6H,7H-pyrrolo[3,2-c]pyridin-4-one (50.00 mg, 0.11 mmol, 1.00 equiv) in EtOH (1.10 mL) was added EtONa (23.31 mg, 0.33 mmol, 3.00 equiv) at 0° C. and stirred for 1 h. The resulting mixture was concentrated under reduced pressure and purified by Prep-HPLC with the following conditions (Column: XBridge Prep OBD C18 Column, 30*150 mm, 5|Ìm; Mobile Phase A: Water (10 mmol/L NH₄HCO₃), Mobile Phase B: ACN; Flow rate: 60 mL/min; Gradient: 43% B to 60% B in 8 min, 60% B; Wave Length: 254/220 nm; RT1 (min): 7.5) to afford 3-[(3-chloro-2-methoxyphenyl)amino]-2-(6-ethoxy-1,5-naphthyridin-4-yl)-1H,5H,6H,7H-pyrrolo[3,2-c]pyridin-4-one (14.00 mg, 26.14%) as a light yellow solid.

LC-MS: (M+H)⁺ found: 464.1.

¹H NMR (400 MHz, DMSO-d6): δ 12.13 (s, 1H), 8.58 (d, J=4.8 Hz, 1H), 8.29 (d, J=9.1 Hz, 1H), 7.76 (s, 1H), 7.50 (d, J=4.9 Hz, 1H), 7.33 (d, J=9.1 Hz, 1H), 7.24-7.17 (m, 1H), 6.79-6.58 (m, 2H), 6.19 (dd, J=7.0, 2.6 Hz, 1H), 4.62 (q, J=7.0 Hz, 2H), 3.86 (s, 3H), 3.47 (td, J=6.8, 2.4 Hz, 2H), 2.95 (t, J=6.8 Hz, 2H), 1.48 (t, J=7.0 Hz, 3H).

### Example 152. 3-[(3-chloro-2-methoxyphenyl)amino]-2-[6-[(2S)-oxetan-2-ylmethoxy]-1,5-naphthyridin-4-yl]-1H,5H,6H, 7H-pyrrolo[3,2-c]pyridin-4-one (compound 735)

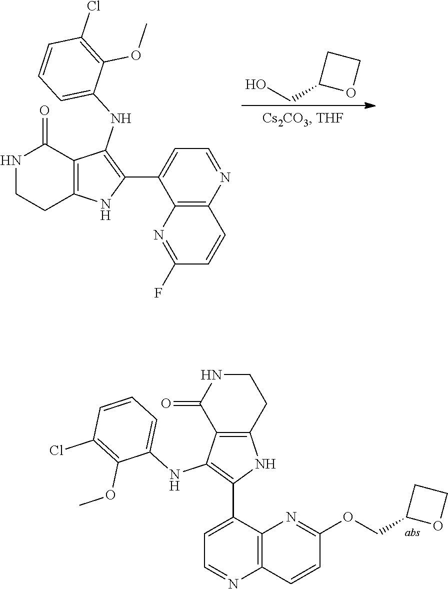 [SMILES: COc1c(Cl)cccc1Nc1c(-c2ccnc3ccc(F)nc23)[nH]c2c1C(=O)NCC2 ; OC[C@@H]1CCO1 ; COc1c(Cl)cccc1Nc1c(-c2ccnc3ccc(OC[C@@H]4CCO4)nc23)[nH]c2c1C(=O)NCC2]

To a solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (65.00 mg, 0.15 mmol, 1.00 equiv) and (2S)-oxetan-2-ylmethanol (65.40 mg, 0.75 mmol, 5.00 equiv) in THF (1.50 mL) were added Cs₂CO₃ (145.10 mg, 0.44 mmol, 3.00 equiv) at room temperature under nitrogen atmosphere. The resulting mixture was stirred for 1 h at room temperature under nitrogen atmosphere. The resulting mixture was concentrated under reduced pressure. The residue was purified by Prep-HPLC with the following conditions (Column; XBridge Prep OBD C18 Column, 30*150 mm, 5|Ìm; Mobile Phase A: Water (10 mmol/L NH₄HCO₃), Mobile Phase B: ACN; Flow rate: 60 mL/min; Gradient: 33% B to 63% B in 7 min; Wave Length: 254 nm; RT1 (min): 6.5) to afford 3-[(3-chloro-2-methoxyphenyl)amino]-2-[6-[(2S)-oxetan-2-ylmethoxy]-1,5-naphthyridin-4-yl]-1H,5H,6H, 7H-pyrrolo[3,2-c]pyridin-4-one (17.40 mg) as a brown solid.

LC-MS: (M+H)⁺ found: 506.1.

¹H NMR (400 MHz, DMSO-d6): δ 12.06 (s, 1H), 8.58 (d, J=4.9 Hz, 1H), 8.32 (d, J=9.1 Hz, 1H), 7.78 (s, 1H), 7.51 (d, J=4.8 Hz, 1H), 7.41 (d, J=9.0 Hz, 1H), 7.20 (t, J=2.6 Hz, 1H), 6.83-6.55 (m, 2H), 6.18 (dd, J=7.8, 1.8 Hz, 1H), 5.26-5.13 (m, 1H), 4.90-4.40 (m, 4H), 3.89 (s, 3H), 3.50 (td, J=6.9, 2.5 Hz, 2H), 3.06-2.88 (m, 2H), 2.84-2.59 (m, 2H).

### Example 153. 3-[(3-chloro-2-methoxyphenyl) amino]-2-[6-[(3,3-difluorocyclobutyl)methoxy]-1,5-naphthyridin-4-yl]-1H,5H,6H,7H-pyrrolo[3,2-c]pyridin-4-one (compound 757)

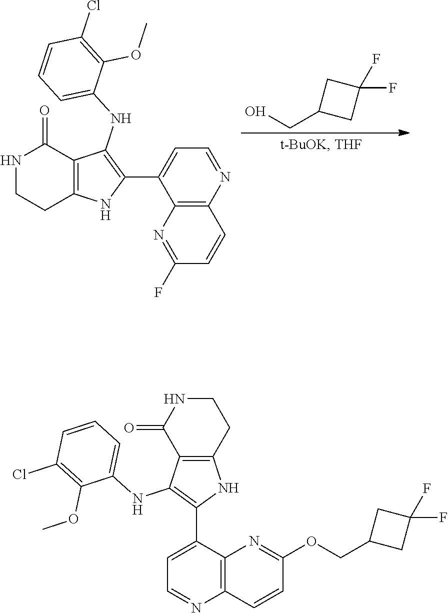 [SMILES: COc1c(Cl)cccc1Nc1c(-c2ccnc3ccc(F)nc23)[nH]c2c1C(=O)NCC2 ; OCC1CC(F)(F)C1 ; COc1c(Cl)cccc1Nc1c(-c2ccnc3ccc(OCC4CC(F)(F)C4)nc23)[nH]c2c1C(=O)NCC2]

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (50.00 mg, 0.11 mmol, 1.00 equiv) and (3,3-difluorocyclobutyl) methanol (69.72 mg, 0.57 mmol, 5.00 equiv) in THF (1 mL) were added t-BuOK (38.44 mg, 0.34 mmol, 3.00 equiv) in portions at 0° C. under nitrogen atmosphere. The resulting solid was dried in an oven under reduced pressure. The crude product (50.00 mg) was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 41% B to 54% B in 7 min, 54% B; Wave Length: 254/220 nm; RT1 (min): 6.37) to afford 3-[(3-chloro-2-methoxyphenyl) amino]-2-[6-[(3,3-difluorocyclobutyl)methoxy]-1,5-naphthyridin-4-yl]-1H,5H,6H,7H-pyrrolo[3,2-c]pyridin-4-one (12.90 mg, 19.98%) as a yellow solid.

LC-MS: (M+H)⁺ found: 416.0.

¹H NMR (400 MHz, DMSO-d₆) δ 12.03 (s, 1H), 8.58 (d, J=4.8 Hz, 1H), 8.30 (d, J=9.0 Hz, 1H), 8.17 (s, 1H), 7.77 (s, 1H), 7.51 (d, J=4.8 Hz, 1H), 7.35 (d, J=9.1 Hz, 1H), 7.22 (d, J=2.6 Hz, 1H), 6.77-6.62 (m, 2H), 6.17-6.15 (m, 1H), 4.66 (d, J=5.9 Hz, 2H), 3.88 (s, 3H), 3.47 (td, J=6.9, 2.5 Hz, 2H), 2.94 (t, J=6.8 Hz, 2H), 2.79-2.77 (m, 3H), 2.64-2.49 (m, 1H).

### Example 154. 3-[(3-chloro-2-methoxyphenyl)amino]-2-[6-(2,2,2-trifluoroethoxy)-1,5-naphthyridin-4-yl]-1H,5H,6H,7H-pyrrolo[3,2-c]pyridin-4-one (compound 763)

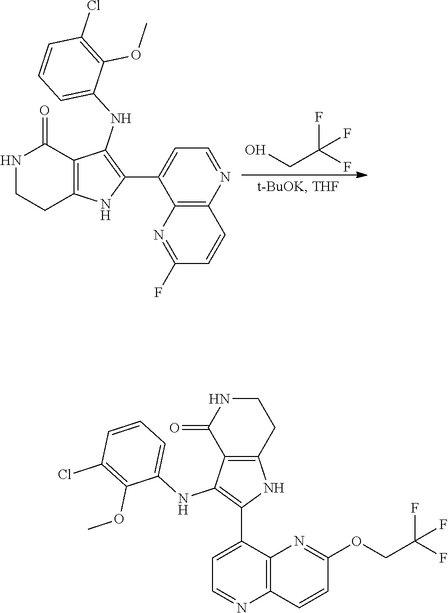

A solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (44.00 mg, 0.10 mmol, 1.00 equiv) in THF (2 mL) was added trifluoroethanol (50.00 mg, 0.50 mmol, 5.00 equiv) and t-BuOK (34.00 mg, 0.30 mmol, 3.00 equiv) dropwise at 0° C. The resulting mixture was stirred for 1 h at room temperature. The reaction was monitored by LCMS. The crude product was purified by Prep-HPLC with the following conditions (Column: XBridge Prep OBD C18 Column, 30*150 mm, 5|Ìm; Mobile Phase A: Water (10 mmol/L NH₄HCO₃), Mobile Phase B: ACN; Flow rate: 60 mL/min; Gradient: 40% B to 70% B in 7 min; Wave Length: 254 nm; RT1 (min): 6.5) to afford 3-[(3-chloro-2-methoxyphenyl)amino]-2-[6-(2,2,2-trifluoroethoxy)-1,5-naphthyridin-4-yl]-1H,5H,6H,7H-pyrrolo[3,2-c]pyridin-4-one (14.70 mg) as a yellow solid.

LC-MS: (M+H)⁺ found: 518.0.

¹H NMR (300 MHz, DMSO-d₆) δ 11.76 (s, 1H), 8.63 (d, J=4.8 Hz, 1H), 8.39 (d, J=6.3 Hz, 1H), 7.82 (s, 1H), 7.55-7.47 (m, 2H), 7.23 (s, 1H), 6.69-6.57 (m, 2H), 6.13 (d, J=6.9 Hz, 1H), 5.37-6.28 (m, 2H), 3.90 (s, 3H), 3.49-3.45 (m, 2H), 2.93 (t, J=6.6 Hz, 2H).

### Example 155. 3-[(3-chloro-2-methoxyphenyl)amino]-2-[6-(cyclopropylmethoxy)-1,5-naphthyridin-4-yl]-1H,5H,6H,7H-pyrrolo[3,2-c]pyridin-4-one (compound 755)

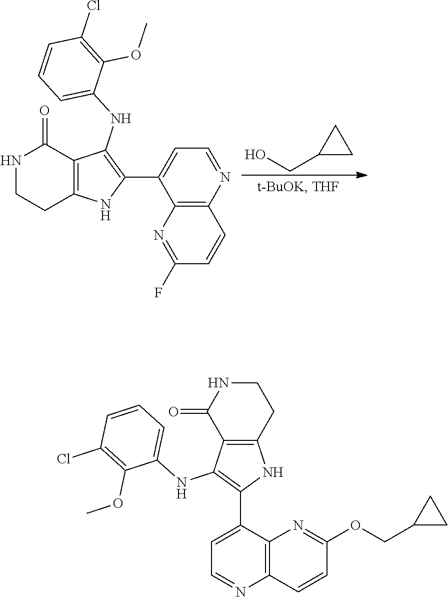

To a solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (60.00 mg, 0.14 mmol, 1.00 equiv) and cyclopropylmethanol (49.40 mg, 0.69 mmol, 5.00 equiv) in DMF (1.50 mL) were added t-BuOK (16.91 mg, 0.15 mmol, 1.10 equiv) dropwise at 0° C. under nitrogen atmosphere. The resulting mixture was stirred for 1 h at 0° C. under nitrogen atmosphere. The mixture was purified by Prep-HPLC with the following conditions (Column: XBridge Prep C18 OBD Column, 30*100 mm, 5|Ìm; Mobile Phase A: Water (10 mmol/L NH₄HCO₃), Mobile Phase B: ACN; Flow rate: 60 mL/min; Gradient: 43% B to 68% B in 7 min, 68% B; Wave Length: 254/220 nm; RT1 (min): 6.32) to afford 3-[(3-chloro-2-methoxyphenyl)amino]-2-[6-(cyclopropylmethoxy)-1,5-naphthyridin-4-yl]-1H,5H,6H,7H-pyrrolo[3,2-c]pyridin-4-one (16.30 mg, 24.04%) as a yellow solid.

LC-MS: (M+H)⁺ found: 490.0.

¹H NMR (400 MHz, DMSO-d₆): δ 12.14 (s, 1H), 8.57 (d, J=4.9 Hz, 1H), 8.29 (d, J=9.1 Hz, 1H), 7.74 (s, 1H), 7.50 (d, J=4.8 Hz, 1H), 7.37 (d, J=9.1 Hz, 1H), 7.23-7.18 (m, 1H), 6.84-6.56 (m, 2H), 6.16 (dd, J=7.8, 1.9 Hz, 1H), 4.43 (d, J=7.1 Hz, 2H), 3.88 (s, 3H), 3.47 (td, J=6.9, 2.5 Hz, 2H), 2.92 (t, J=6.8 Hz, 2H), 1.48-1.30 (m, 1H), 0.73-0.56 (m, 2H), 0.49-0.33 (m, 2H).

### Example 156. 3-[(3-chloro-2-methoxyphenyl)amino]-2-[6-[(1-fluorocyclopropyl)methoxy]-1,5-naphthyridin-4-yl]-1H,5H,6H,7H-pyrrolo[3,2-c]pyridin-4-one (compound 759)

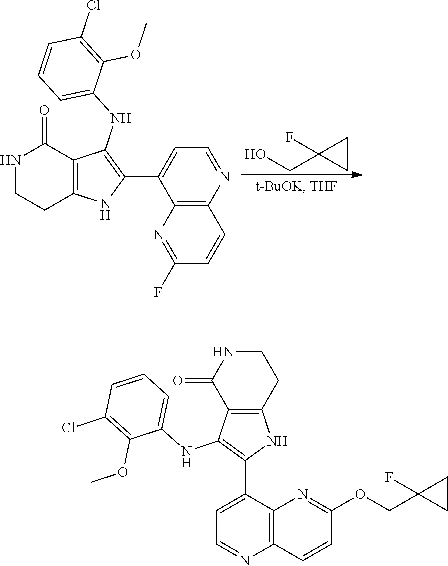

To a solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (70.00 mg, 0.16 mmol, 1.00 equiv) and (1-fluorocyclopropyl)methanol (72.02 mg, 0.80 mmol, 5.00 equiv) in DMF (2 mL) were added t-BuOK (19.73 mg, 0.18 mmol, 1.10 equiv) dropwise at 0° C. under nitrogen atmosphere. The resulting mixture was stirred for 1 h at 0° C. under nitrogen atmosphere. The mixture was purified by Prep-HPLC with the following conditions (Column: XBridge Prep C18 OBD Column, 30*100 mm, 5|Ìm; Mobile Phase A: Water (10 mmol/L NH₄HCO₃), Mobile Phase B: ACN; Flow rate: 60 mL/min; Gradient: 37% B to 57% B in 10 min, 57% B; Wave Length: 254/220 nm; RT1 (min): 8.85) to afford 3-[(3-chloro-2-methoxyphenyl)amino]-2-[6-[(1-fluorocyclopropyl)methoxy]-1,5-naphthyridin-4-yl]-1H,5H,6H,7H-pyrrolo[3,2-c]pyridin-4-one (16.70 mg, 20.50%) as a yellow solid.

LC-MS: (M+H)⁺ found: 508.0.

¹H NMR (400 MHz, DMSO-d₆): δ 11.94 (s, 1H), 8.59 (d, J=4.8 Hz, 1H), 8.33 (d, J=9.1 Hz, 1H), 7.77 (s, 1H), 7.52 (d, J=4.9 Hz, 1H), 7.44 (d, J=9.1 Hz, 1H), 7.25-7.17 (m, 1H), 6.80-6.51 (m, 2H), 6.15 (dd, J=7.9, 1.7 Hz, 1H), 4.93 (d, J=23.3 Hz, 2H), 3.88 (s, 3H), 3.47 (td, J=6.9, 2.5 Hz, 2H), 2.93 (t, J=6.8 Hz, 2H), 1.29-1.11 (m, 2H), 1.03-0.88 (m, 2H).

### Example 157 3-[(3-chloro-2-methoxyphenyl)amino]-2-[6-(cyclobutylmethoxy)-1,5-naphthyridin-4-yl]-1H,5H,6H,7H-pyrrolo[3,2-c]pyridin-4-one (compound 756)

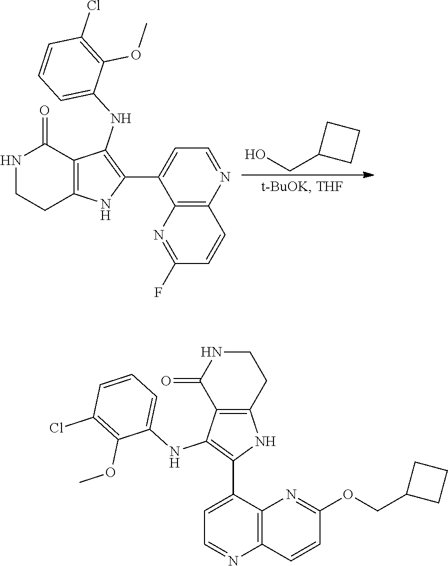

To a solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (80.00 mg, 0.18 mmol, 1.00 equiv) and cyclobutylmethanol (78.69 mg, 0.91 mmol, 5.00 equiv) in DMF (1.80 mL) were added t-BuOK (22.55 mg, 0.20 mmol, 1.10 equiv) dropwise at 0° C. under nitrogen atmosphere. The resulting mixture was stirred for 1 h at 0° C. under nitrogen atmosphere. The mixture was purified by Prep-HPLC with the following conditions (Column: XBridge Prep C18 OBD Column, 30*100 mm, 5|Ìm; Mobile Phase A: Water (10 mmol/L NH₄HCO₃), Mobile Phase B: ACN; Flow rate: 60 mL/min; Gradient: 40% B to 60% B in 10 min, 60% B; Wave Length: 254/220 nm; RT1 (min): 10.38) to afford 3-[(3-chloro-2-methoxyphenyl)amino]-2-[6-(cyclobutylmethoxy)-1,5-naphthyridin-4-yl]-1H,5H,6H,7H-pyrrolo[3,2-c]pyridin-4-one (19.20 mg, 19.95%) as a yellow solid.

LC-MS: (M+H)⁺ found: 504.1.

¹H NMR (400 MHz, DMSO-d₆): δ 12.18 (s, 1H), 8.57 (d, J=4.9 Hz, 1H), 8.28 (d, J=9.1 Hz, 1H), 7.75 (s, 1H), 7.51 (d, J=4.9 Hz, 1H), 7.34 (d, J=9.1 Hz, 1H), 7.23-7.19 (m, 1H), 6.77-6.58 (m, 2H), 6.17 (dd, J=7.8, 1.9 Hz, 1H), 4.57 (d, J=6.9 Hz, 2H), 3.88 (s, 3H), 3.48 (td, J=6.9, 2.5 Hz, 2H), 3.04-2.78 (m, 3H), 2.23-2.07 (m, 2H), 2.02-1.81 (m, 4H).

### Example 158. 3-[(3-chloro-2-methoxyphenyl)amino]-2-(6-cyclobutoxy-1,5-naphthyridin-4-yl)-1H,5H,6H,7H-pyrrolo[3,2-c] pyridin-4-one (compound 768)

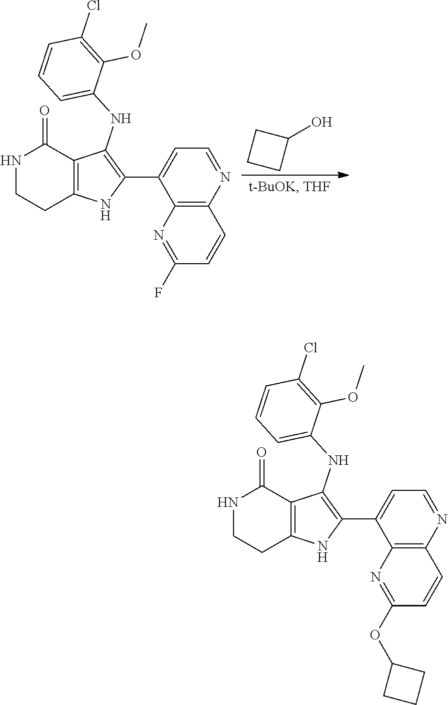

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (10.00 mg, 0.02 mmol, 1.00 equiv) and cyclobutanol (8.23 mg, 0.11 mmol, 5.00 equiv) in DMF (0.50 mL) were added t-BuOK (2.82 mg, 0.03 mmol, 1.10 equiv) in portions at degrees 0° C. under nitrogen atmosphere. The crude product (60.00 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 46% B to 76% B in 7 min; Wave Length: 254 nm; RT1 (min): 6.5) to afford 3-[(3-chloro-2-methoxyphenyl)amino]-2-(6-cyclobutoxy-1,5-naphthyridin-4-yl)-1H,5H,6H,7H-pyrrolo[3,2-c] pyridin-4-one (9.90 mg, 17.59%) as a yellow solid.

LC-MS: (M+H)⁺ found: 490.0.

¹H NMR (400 MHz, DMSO-d₆) δ 12.31 (s, 1H), 8.58 (d, J=4.9 Hz, 1H), 8.30 (d, J=9.1 Hz, 1H), 7.70 (s, 1H), 7.51 (d, J=4.9 Hz, 1H), 7.33 (d, J=9.0 Hz, 1H), 7.24 (s, 1H), 6.75-6.65 (m, 2H), 6.17-6.15 (m, 1H), 5.53 (p, J=7.2 Hz, 1H), 3.90 (s, 3H), 3.54-3.42 (m, 2H), 3.00 (t, J=6.8 Hz, 2H), 2.51 (p, J=1.8 Hz, 2H), 2.35-2.13 (m, 2H), 1.99-1.68 (m, 2H).

### Example 159. 3-[(3-chloro-2-methoxyphenyl) amino]-2-[6-(1-methylcyclopropoxy)-1,5-naphthyridin-4-yl]-1H,5H,6H,7H-pyrrolo[3,2-c]pyridin-4-one (compound 767)

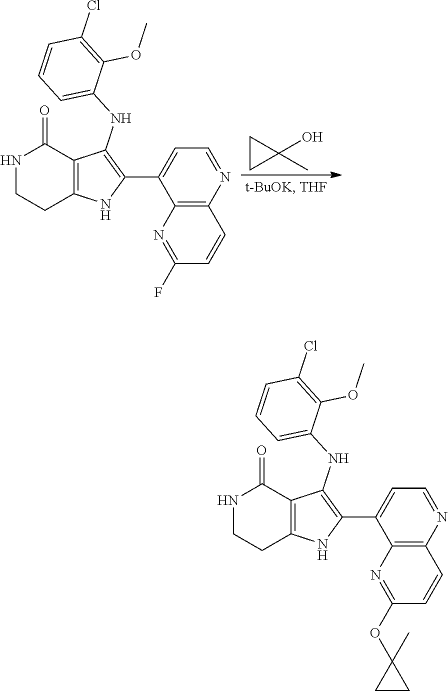

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (80.00 mg, 0.18 mmol, 1.00 equiv) and 1-methylcyclopropan-1-ol (131.74 mg, 1.83 mmol, 10.00 equiv) in DMF (1.2 mL) were added t-BuOK (24.60 mg, 0.22 mmol, 1.20 equiv) in portions at 0° C. under nitrogen atmosphere. The resulting mixture was stirred for additional 3 h at 50° C. The crude product (80.00 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 45% B to 75% B in 7 min; Wave Length: 254 nm; RT1 (min): 6.5) to afford 3-[(3-chloro-2-methoxyphenyl) amino]-2-[6-(1-methylcyclopropoxy)-1,5-naphthyridin-4-yl]-1H,5H,6H,7H-pyrrolo[3,2-c]pyridin-4-one (15.00 mg, 16.54%) as a yellow solid.

LC-MS: (M+H)⁺ found: 490.0.

¹H NMR (400 MHz, DMSO-d6) δ 12.95 (s, 1H), 8.57 (d, J=4.9 Hz, 11H), 8.31 (d, J=9.1 Hz, 1H), 7.78 (s, 1H), 7.51 (d, J=4.9 Hz, 1H), 7.33-7.26 (m, 2H), 6.87-6.63 (m, 2H), 6.21-6.19 (m, 1H), 3.97 (s, 3H), 3.48 (td, J=6.7, 2.4 Hz, 2H), 3.01 (t, J=6.8 Hz, 2H), 1.80 (s, 3H), 1.26-1.15 (m, 2H), 1.07-0.95 (m, 2H).

### Example 160. 3-[(3-chloro-2-methoxyphenyl)amino]-2-[6-(3,3-difluorocyclobutoxy)-1,5-naphthyridin-4-yl]-1H,5H,6H,7H-pyrrolo[3, 2-c] pyridin-4-one (compound 766)

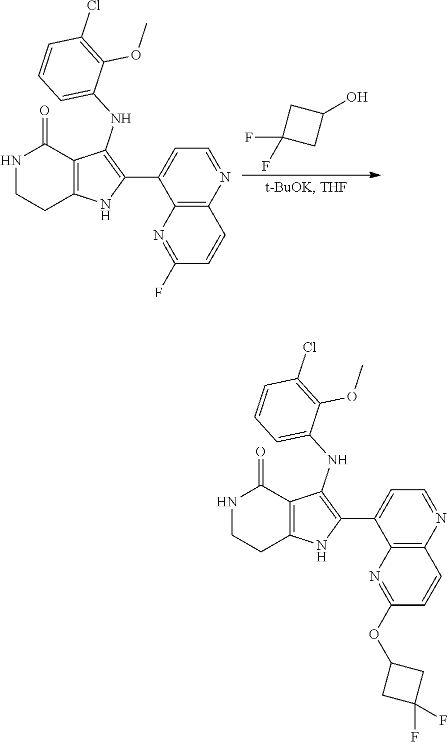

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (80.00 mg, 0.18 mmol, 1.00 equiv) and 3,3-difluorocyclobutan-1-ol (197.48 mg, 1.83 mmol, 10.00 equiv) in DMF (1.20 mL) were added t-BuOK (22.55 mg, 0.20 mmol, 1.10 equiv) in portions at 0° C. under nitrogen atmosphere. The crude product (80.00 mg) was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 37% B to 57% B in 8 min, 57% B; Wave Length: 254/220 nm; RT1 (min): 8) to afford 3-[(3-chloro-2-methoxyphenyl)amino]-2-[6-(3,3-difluorocyclobutoxy)-1,5-naphthyridin-4-yl]-1H,5H,6H,7H-pyrrolo[3, 2-c] pyridin-4-one (13.60 mg, 13.62%) as a yellow solid.

LC-MS: (M+H)⁺ found: 526.0.

¹H NMR (400 MHz, DMSO-d₆) δ 11.90 (s, 1H), 8.62 (d, J=4.9 Hz, 1H), 8.35 (d, J=9.1 Hz, 1H), 7.65 (s, 1H), 7.53 (d, =4.8 Hz, 1H), 7.39 (d, J=9.1 Hz, 1H), 7.20 (d, J=2.7 Hz, 1H), 6.72-6.59 (m, 2H), 6.14-6.12 (m, 1H), 5.58 (d, J=6.4 Hz, 1H), 3.88 (s, 3H), 3.49 (td, J=6.9, 2.6 Hz, 2H), 3.22-3.04 (m, 2H), 3.01-2.80 (m, 4H).

### Example 161. 3-[(3-fluoro-2-methylphenyl)amino]-2-(6-methyl-1,5-naphthyridin-4-yl)-1H,5H,6H,7H-pyrrolo[3,2-c]pyridin-4-one (compound 810)

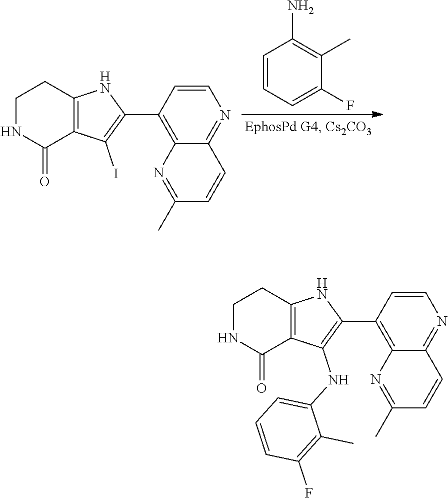

To a stirred solution of 3-iodo-2-(6-methyl-1,5-naphthyridin-4-yl)-1H,5H,6H,7H-pyrrolo[3,2-c]pyridin-4-one (80 mg, 0.19 mmol, 1.00 equiv) and 3-fluoro-2-methyl-aniline (29 mg, 0.23 mmol, 1.2 equiv) in DMF was added Cs₂CO₃ (128.97 mg, 0.396 mmol, 2 equiv) and EPhos Pd G4 (36 mg, 0.04 mmol, 0.2 equiv) in portions at room temperature under argon atmosphere. The resulting mixture was stirred for 3 h at 50° C. under argon atmosphere. Desired product could be detected by LCMS. The resulting mixture was concentrated under vacuum. The residue was purified by Prep-TLC (DCM/MeOH 10:1) to give the crude product. Then the crude product was The crude product (40 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 28% B to 50% B in 9 min, 50% B; Wave Length: 254/220 nm; RT1 (min): 8.85) to afford 3-[(3-fluoro-2-methylphenyl)amino]-2-(6-methyl-1,5-naphthyridin-4-yl)-1H,5H,6H,7H-pyrrolo[3,2-c]pyridin-4-one (28.4 mg, 35.24%) as an orange solid.

LC-MS: (M+H)⁺ found: 402.30.

¹H NMR (300 MHz, DMSO-d6) δ 12.62 (d, 1H), 8.61 (d, 1H), 8.30 (d, 1H), 7.72 (t, 2H), 7.44 (d, 1H), 7.27 (s, 1H), 6.76 (m, 1H), 6.51 (t, 1H), 6.11 (d, 1H), 3.48 (t, 2H), 3.00 (t, 2H), 2.86 (s, 3H), 2.23 (s, 3H).

### Example 162. 3-[(3-fluoro-2-methoxyphenyl)amino]-2-(6-methyl-1,5-naphthyridin-4-yl)-1H,5H,6H,7H-pyrrolo[3,2-c]pyridin-4-one (compound 809)

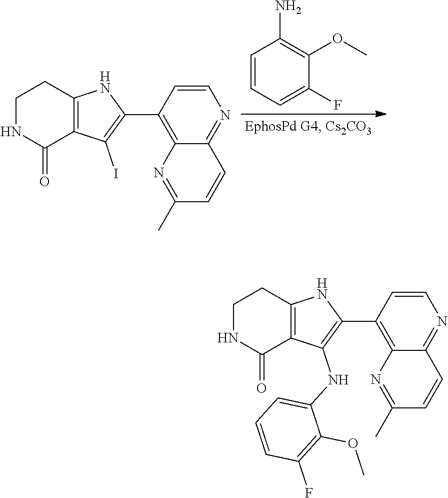

To a stirred solution/mixture of 3-iodo-2-(6-methyl-1,5-naphthyridin-4-yl)-1H,5H,6H,7H-pyrrolo[3,2-c]pyridin-4-one (80 mg, 0.19 mmol, 1.00 equiv) and 3-fluoro-2-methoxyaniline (33 mg, 0.23 mmol, 1.2 equiv) in DMF was added Cs₂CO₃ (128 mg, 0.39 mmol, 2 equiv) and EPhos Pd G4 (36 mg, 0.04 mmol, 0.2 equiv) in portions at room temperature under argon atmosphere. The resulting mixture was stirred for 3 h at 50 degrees C. under argon atmosphere. The resulting mixture was concentrated under vacuum. The residue was purified by Prep-TLC (CH₂Cl₂/MeOH 20:1) and HPLC(Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 30% B to 50% B in 10 min, 50% B; Wave Length: 254/220 nm; RT1 (min): 9.28) to afford 3-[(3-fluoro-2-methoxyphenyl)amino]-2-(6-methyl-1,5-naphthyridin-4-yl)-1H,5H,6H,7H-pyrrolo[3,2-c]pyridin-4-one (37.0 mg, 44.16%) as an orange solid.

LC-MS: (M+H)⁺ found: 418.00.

¹H NMR (300 MHz, DMSO-d₆) δ 12.48 (d, 1H), 8.68 (d, 1H), 8.32 (d, 1H), 8.05 (s, 1H), 7.75 (d, 1H), 7.53 (s, 1H), 7.21 (s, 1H), 6.65 (m, 1H), 6.52 (m, 1H), 6.08 (d, 1H), 3.85 (s, 3H), 3.46 (t, 2H), 2.99 (t, 2H), 2.67 (s, 3H).

### Example 163. 3-[(3-chloro-2-methoxyphenyl)amino]-2-{2-cyclopropoxypyrido[3,2-d]pyrimidin-8-yl}-1H,5H,6H,7H-pyrrolo[3,2-c]pyridin-4-one (compound 771)

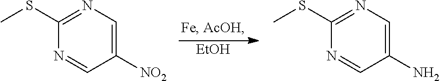

To a stirred solution of 2-(methylsulfanyl)-5-nitropyrimidine (5 g, 29.21 mmol, 1.00 equiv) in EtOH (200 mL) was added AcOH (120 mL) and Fe (17 g, 292.11 mmol, 10 equiv) in portions at rt under N2 atmosphere. The resulting mixture was stirred for 2 h at 80 degrees C. under N₂ atmosphere. Desired product could be detected by LCMS. LC-MS: M+H found: 142. The resulting mixture was concentrated under reduced pressure. The resulting mixture was extracted with EA (3×100 mL). The combined organic layers were washed with Saturated NaCl (3×100 mL), dried over anhydrous Na₂SO₄. After filtration, the filtrate was concentrated under reduced pressure to afford 2-(methylsulfanyl)pyrimidin-5-amine (3.5 g, 84.86%) as a yellow solid.

LC-MS: M+H found: 142.0.

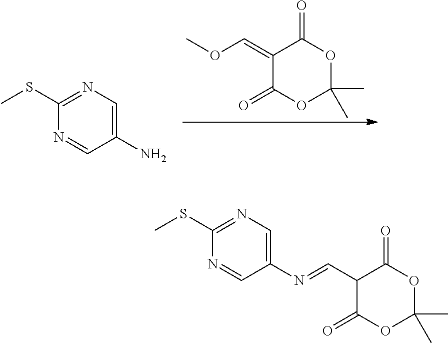

To a stirred solution of 2-(methylsulfanyl)pyrimidin-5-amine (3.2 g, 22.66 mmol, 1.00 equiv) and 5-(methoxymethylidene)-2,2-dimethyl-1,3-dioxane-4,6-dione (5.06 g, 27.18 mmol, 1.20 equiv) in DMF (80.00 mL) at rt under N2 atmosphere. The resulting mixture was stirred for 1 h at 80 degrees C. under N2atmosphere. Desired product could be detected by LCMS. The resulting mixture was added MeOH (50 mL). The resulting mixture was filtered, the filter cake was washed with MeOH (3×20 mL). The filter cake was concentrated under reduced pressure to afford 2,2-dimethyl-5-[(1E)-[[2-(methylsulfanyl)pyrimidin-5-yl]imino]methyl]-1,3-dioxane-4,6-dione (5.4 g, 80.68%) as a yellow solid.

LC-MS: (M+H)⁺ found: 296.0.

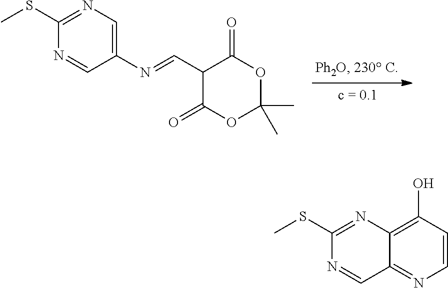

To a stirred solution of 2,2-dimethyl-5-[(1E)-([2-(methylsulfanyl)pyrimidin-5-yl]imino)methyl]-1,3-dioxane-4,6-dione (5.3 g, 17.95 mmol, 1.00 equiv) in phenoxybenzene (360 mL) at rt under N2 atmosphere. The resulting mixture was stirred at 230 degrees C. under N2 atmosphere. Desired product could be detected by LCMS. LC-MS: M+H found: 194. The reaction was addition of Hexane (700 ml) at rt. The resulting mixture was filtered; the filter cake was washed with Hexane (3×200 ml). The filtrate was concentrated under reduced pressure.

LC-MS: (M+H)⁺ found: 194.0.

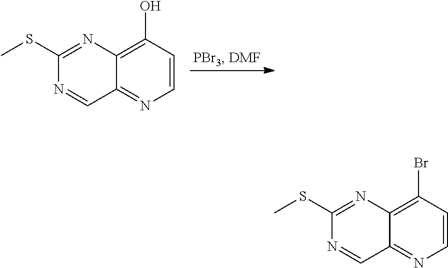

To a stirred solution of 2-(methylsulfanyl)pyrido[3,2-d]pyrimidin-8-ol (2.8 g, 14.49 mmol, 1.00 equiv) in DMF (80 mL) was added PBr₃ (4.3 g, 15.94 mmol, 1.1 equiv) dropwise at 0 degrees C. under N2 atmosphere. The resulting mixture was stirred for 1 h at rt under N2 atmosphere. Desired product could be detected by LCMS. LC-MS: M+H found: 256. The resulting mixture was extracted with EA (3×100 mL). The combined organic layers were washed with aq. NaCl (3×100 mL), dried over anhydrous Na₂SO₄. After filtration, the filtrate was concentrated under reduced pressure. The residue was purified by silica gel column chromatography, eluted with PE:EA (4:1) to afford 8-bromo-2-(methylsulfanyl)pyrido[3,2-d]pyrimidine (1.6 g, 43.11%) as a white solid.

LC-MS: (M+H)⁺ found: 256.0.

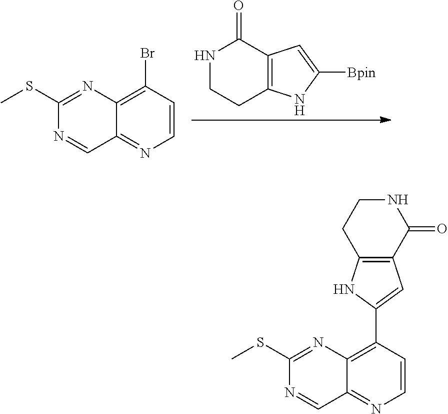

To a stirred solution of 8-bromo-2-(methylsulfanyl)pyrido[3,2-d]pyrimidine (700 mg, 2.73 mmol, 1.00 equiv) and 2-(4,4,5,5-tetramethyl-1,3,2-dioxaborolan-2-yl)-1H,5H,6H,7H-pyrrolo[3,2-c]pyridin-4-one (1075 mg, 4.10 mmol, 1.50 equiv) in dioxane (10 mL) and H₂O (1 mL) were added Na₂CO₃ (869 mg, 8.19 mmol, 3.00 equiv) and XPhos Pd G2 (215 mg, 0.27 mmol, 0.10 equiv) dropwise/in portions at rt under N₂ atmosphere. The resulting mixture was stirred for 2 h at 80 degrees C. under N₂ atmosphere. Desired product could be detected by LCMS. The resulting mixture was concentrated under vacuum. The residue was purified by silica gel column chromatography, eluted with DCM/MeOH=24:1 to afford 2-[2-(methylsulfanyl)pyrido[3,2-d]pyrimidin-8-yl]-1H,5H,6H,7H-pyrrolo[3,2-c]pyridin-4-one (700 mg, 82.26%) as a yellow solid.

LC-MS: (M+H)⁺ found 312.0.

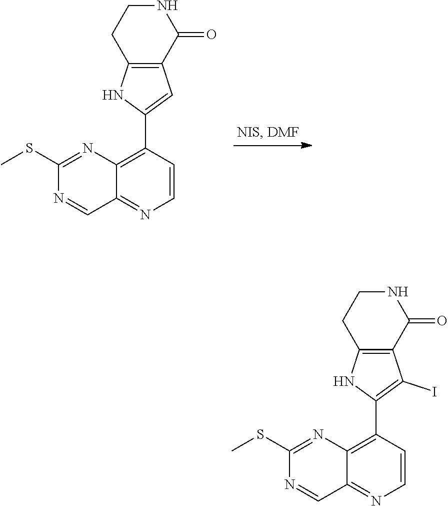 [SMILES: CSc1ncc2nccc(-c3cc4c([nH]3)CCNC4=O)c2n1 ; CSc1ncc2nccc(-c3[nH]c4c(c3I)C(=O)NCC4)c2n1]

To a stirred solution of 2-[2-(methylsulfanyl)pyrido[3,2-d]pyrimidin-8-yl]-1H,5H,6H,7H-pyrrolo[3,2-c]pyridin-4-one (600 mg, 1.93 mmol, 1.00 equiv) and NIS (650 mg, 2.89 mmol, 1.50 equiv) in DMF (10 mL) at rt under N2 atmosphere. The resulting mixture was stirred for overnight at 30 degrees C. under N2 atmosphere. Desired product could be detected by LCMS. LC-MS: M+H found: 438. The reaction was quenched by the addition of Saturated aq. Na₂SO₃ (20 mL) at 0 degrees C. The precipitated solids were collected by filtration and washed with H₂O (20 mL×3). The residue was purified by silica gel column chromatography, eluted with DCM:MeOH=10:1 to afford 3-iodo-2-[2-(methylsulfanyl)pyrido[3,2-d]pyrimidin-8-yl]-1H,5H,6H,7H-pyrrolo[3,2-c]pyridin-4-one (650 mg, 77.14%) as a yellow solid.

LC-MS: (M+H)⁺ found: 438.0.

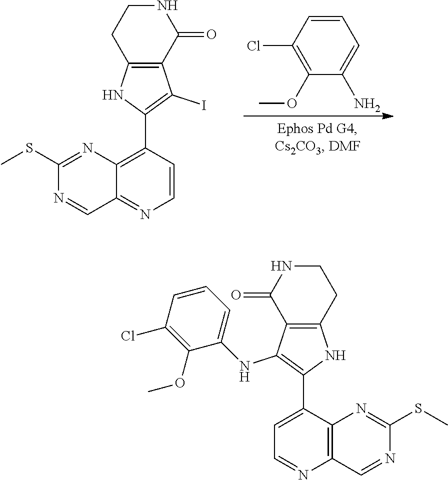

To a stirred solution of 3-iodo-2-[2-(methylsulfanyl)pyrido[3,2-d]pyrimidin-8-yl]-1H,5H,6H,7H-pyrrolo[3,2-c]pyridin-4-one (370 mg, 0.85 mmol, 1.00 equiv) and 3-chloro-2-methoxyaniline (134 mg, 0.85 mmol, 1 equiv) in DMF (4 mL) were added EPhos Pd G4 (78 mg, 0.08 mmol, 0.1 equiv) and Cs₂CO₃ (827 mg, 2.54 mmol, 3 equiv) in portions at rt under Ar atmosphere. The resulting mixture was stirred for 1.5 h at 50 degrees C. under Ar atmosphere. Desired product could be detected by LCMS. The resulting mixture was concentrated under vacuum. The residue was purified by silica gel column chromatography, eluted with DCM:MeOH (10:1) to afford 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (220 mg, 55.68%) as an orange solid.

LC-MS: (M+H)⁺ found: 467.0.

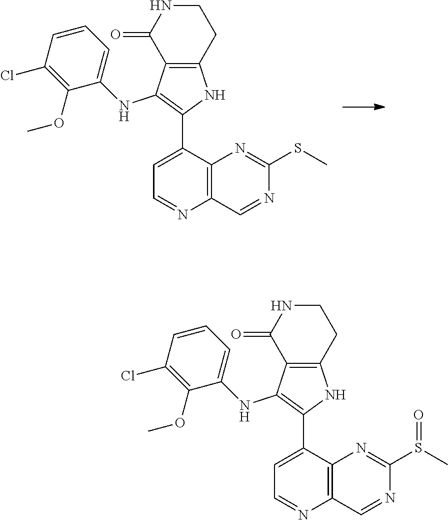 [SMILES: COc1c(Cl)cccc1Nc1c(-c2ccnc3cnc(SC)nc23)[nH]c2c1C(=O)NCC2 ; COc1c(Cl)cccc1Nc1c(-c2ccnc3cnc(S(C)=O)nc23)[nH]c2c1C(=O)NCC2]

To a stirred solution of 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (50 mg, 0.11 mmol, 1.00 equiv) in DCM (2 mL, 31.46 mmol, 293.80 equiv) were added MCPBA (29 mg, 0.12 mmol, 1.1 equiv) dropwise/in portions at 0 degrees C. under N₂ atmosphere. The resulting mixture was stirred for 1 h at 0 degrees C. under N₂ atmosphere.

Desired product could be detected by LCMS. LC-MS: M+H found: 483. The resulting mixture was extracted with DCM (3×4 mL). The combined organic layers were dried over anhydrous Na₂SO₄. After filtration, the filtrate was concentrated under reduced pressure. The crude product was used in the next step directly without further purification.

LC-MS: (M+H)⁺ found: 483.0.

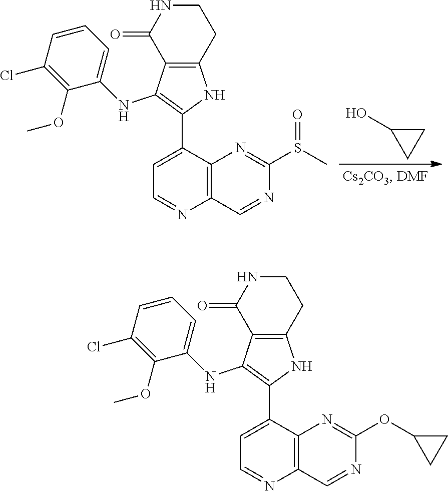

To a stirred solution of 3-[(3-chloro-2-methoxyphenyl)amino]-2-{2-methanesulfinylpyrido[3,2-d]pyrimidin-8-yl}-1H,5H,6H,7H-pyrrolo[3,2-c]pyridin-4-one (100 mg, 0.20 mmol, 1.00 equiv) and Cs₂CO₃ (162 mg, 0.49 mmol, 2.4 equiv) in DMF (2 mL) were added cyclopropanol (14 mg, 0.25 mmol, 1.2 equiv) dropwise at 0 degrees C. under N₂ atmosphere. The resulting mixture was stirred for 1 h at rt under N2 atmosphere. The resulting mixture was extracted with DCM:MeOH=10:1 (3×5 mL). The combined organic layers were washed with aq. NaCl (3×10 ml), dried over anhydrous Na₂SO₄. After filtration, the filtrate was concentrated under reduced pressure and dissolved in DMF. The crude product (50 mg) was purified by Prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 42% B to 47% B in 10 min, 47% B; Wave Length: 220/254 nm; RT1 (min): 9.13) to afford 3-[(3-chloro-2-methoxyphenyl)amino]-2-{2-cyclopropoxypyrido[3,2-d]pyrimidin-8-yl}-1H,5H,6H,7H-pyrrolo[3,2-c]pyridin-4-one (8.6 mg, 8.71%) as a yellow solid.

LC-MS: (M+H)⁺ found: 477.25.

¹H NMR (400 MHz, DMSO-d₆) δ 12.48 (s, 1H), 9.51 (s, 1H), 8.71 (d, J=4.8 Hz, 1H), 7.91 (s, 1H), 7.54 (d, J=4.9 Hz, 1H), 7.33 (d, J=2.6 Hz, 1H), 6.79 (m, J=8.1, 1.7 Hz, 1H), 6.75 (t, J=8.0 Hz, 1H), 6.17 (m, J=7.9, 1.8 Hz, 1H), 4.69-4.60 (m, 1H), 3.95 (s, 3H), 3.54-3.42 (m, 2H), 2.99 (t, J=6.8 Hz, 2H), 1.00-0.94 (m, 4H).

### Example 164. 3-[(3-chloro-2-methoxyphenyl)amino]-2-(6-methoxyquinolin-4-yl)-1H,5H,6H,7H-pyrrolo[3,2-c]pyridin-4-one (compound 773)

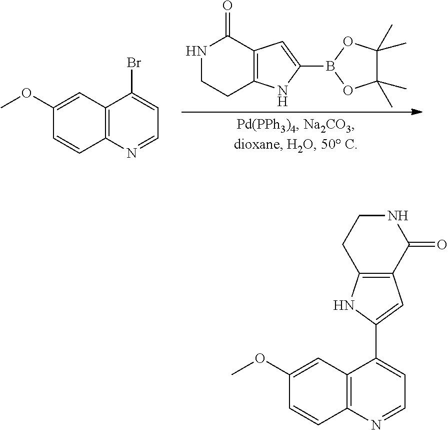 [SMILES: COc1ccc2nccc(Br)c2c1 ; CC1(C)OB(c2cc3c([nH]2)CCNC3=O)OC1(C)C ; COc1ccc2nccc(-c3cc4c([nH]3)CCNC4=O)c2c1]

To a stirred mixture of 4-bromo-6-methoxyquinoline (250 mg, 1.05 mmol, 1.00 equiv) and 2-(4,4,5,5-tetramethyl-1,3,2-dioxaborolan-2-yl)-1H,5H,6H,7H-pyrrolo[3,2-c]pyridin-4-one (412 mg, 1.57 mmol, 1.50 equiv) in dioxane was added Na₂CO₃ (333 mg, 3.15 mmol, 3.00 equiv) and Pd(PPh₃)₄ (121 mg, 0.10 mmol, 0.10 equiv) in portions at room temperature under nitrogen atmosphere. To the above mixture was added H₂O (1.5 mL, 83.26 mmol, 79.29 equiv) dropwise at room temperature. The resulting mixture was stirred for additional 2 h at room temperature. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford 2-(6-methoxyquinolin-4-yl)-1H,5H,6H,7H-pyrrolo[3,2-c]pyridin-4-one (250 mg, 81.17%) as a yellow solid.

LC-MS: (M+H)⁺ found: 294.1.

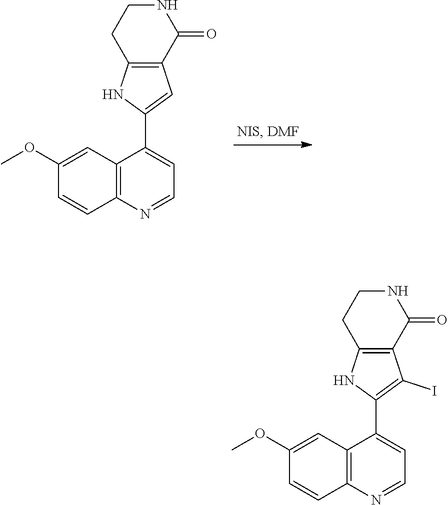 [SMILES: COc1ccc2nccc(-c3cc4c([nH]3)CCNC4=O)c2c1 ; COc1ccc2nccc(-c3[nH]c4c(c3I)C(=O)NCC4)c2c1]

To a stirred solution of 2-(6-methoxyquinolin-4-yl)-1H,5H,6H,7H-pyrrolo[3,2-c]pyridin-4-one (250 mg, 0.85 mmol, 1.00 equiv) in DMF (10.00 mL) was added N-iodosuccinimide (210 mg, 0.93 mmol, 1.10 equiv) in portions at 0 degrees C. under nitrogen atmosphere. The resulting mixture was stirred for overnight at room temperature under nitrogen atmosphere. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford 3-iodo-2-(6-methoxyquinolin-4-yl)-1H,5H,6H,7H-pyrrolo[3,2-c]pyridin-4-one (220 mg, 61.57%) as a yellow solid.

LC-MS: (M+H)⁺ found: 419.95.

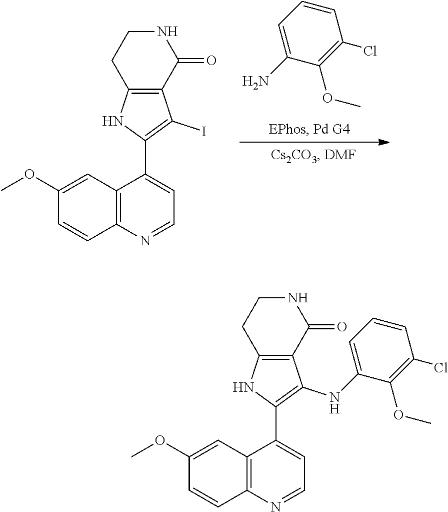

To a stirred solution of 3-iodo-2-(6-methoxyquinolin-4-yl)-1H,5H,6H,7H-pyrrolo[3,2-c]pyridin-4-one (100 mg, 0.23 mmol, 1.00 equiv) in DMF (2.00 mL) was added Cs₂CO₃ (155 mg, 0.47 mmol, 2.00 equiv) and EPhos Pd G4 (43 mg, 0.04 mmol, 0.20 equiv) at room temperature under nitrogen atmosphere. To the above mixture was added 3-chloro-2-methoxyaniline (37 mg, 0.23 mmol, 1.00 equiv) at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford Product 60 mg (crude). The crude product (60 mg) was purified by Prep-HPLC with the following conditions (Column. YMC-Actus Triart C18 ExRS, 30*250 mm, 5|Ìm; Mobile Phase A: Water (10 mmol/L NH4HCO3), Mobile Phase B: ACN; Flow rate: 60 mL/min; Gradient: 24% B to 44% B in 10 min, 44% B; Wave Length: 254/220 nm; RT1 (min): 10.85) to afford 3-[(3-chloro-2-methoxyphenyl)amino]-2-(6-methoxyquinolin-4-yl)-1H,5H,6H,7H-pyrrolo[3,2-c]pyridin-4-one (39.7 mg, 37.07%) as a yellow solid.

LC-MS: (M+H)⁺ found: 449.

¹H NMR (400 MHz, DMSO-d6) δ 11.73 (s, 1H), 8.70 (d, J=4.0 Hz, 1H), 7.86 (d, J=8.0 Hz, 1H), 7.66 (s, 1H), 7.45 (d, J=4.0 Hz, 1H), 7.37-7.31 (m, 2H), 7.19 (s, 1H), 6.48-6.45 (m, 1H), 6.35 (t, J=8.0 Hz, 1H), 6.07-6.07 (m, 1H), 3.76 (s, 6H), 3.49-3.45 (m, 2H), 2.88 (t, J=8 Hz, 2H).

### Example 165. 3-[(3-chloro-2-methoxyphenyl)amino]-2-{2-cyclopropylpyrido[3,2-d]pyrimidin-8-yl}-1H,5H,6H,7H-pyrrolo[3,2-c]pyridin-4-one (compound 776)

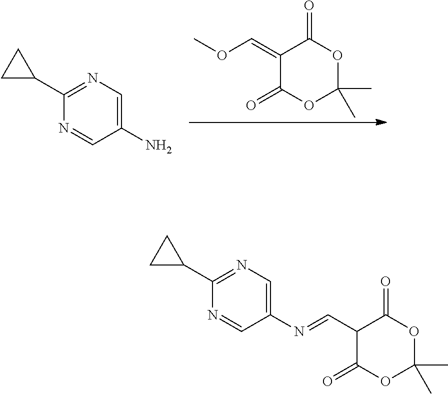

To a stirred solution of 2-cyclopropylpyrimidin-5-amine (1.5 g, 11.09 mmol, 1.00 equiv) in DMF (10.00 mL) was added 5-(methoxymethylidene)-2,2-dimethyl-1,3-dioxane-4,6-dione (2.27 g, 12.20 mmol, 1.10 equiv) dropwise at room temperature under argon atmosphere. The resulting mixture was stirred for 2 h at 80° C. under air atmosphere. The mixture was allowed to cool down to room temperature. The resulting mixture was diluted with MeOH (20 mL). The precipitated solids were collected by filtration and washed with MeOH (3×10 mL). The resulting mixture was concentrated under reduced pressure to obtain 5-[(1E)-[(2-cyclopropylpyrimidin-5-yl)imino]methyl]-2,2-dimethyl-1,3-dioxane-4,6-dione (3 g, 93.45%) as a yellow solid.

LC-MS: (M+H)⁺ found: 289.90

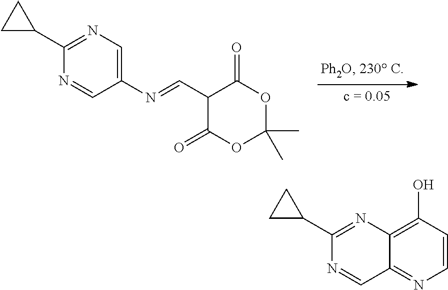

A solution of diphenyl-ether (50 mL) was stirred at 220° C. under argon atmosphere. To the above solution was added 5-[(1E)-[(2-cyclopropylpyrimidin-5-yl)imino]methyl]-2,2-dimethyl-1,3-dioxane-4,6-dione (3 g, 10.37 mmol, 1.00 equiv) dropwise over 10 min at 220° C. The resulting mixture was stirred for additional 5 min at 220° C. The mixture was allowed to cool down to room temperature. The resulting mixture was diluted with hexane (200 mL) at 0° C. The precipitated solids were collected by filtration and washed with hexane (3×20 mL). The resulting mixture was concentrated under reduced pressure to obtain 2-cyclopropylpyrido[3,2-d]pyrimidin-8-ol (1.7 g, crude) as a brown solid.

LC-MS: (M+H)⁺ found: 290.00.

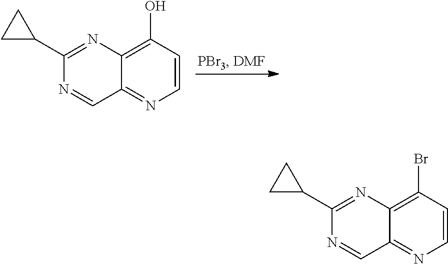 [SMILES: Oc1ccnc2cnc(C3CC3)nc12 ; Brc1ccnc2cnc(C3CC3)nc12]

To a stirred solution of 2-cyclopropylpyrido[3,2-d]pyrimidin-8-ol (800 mg, 4.27 mmol, 1.00 equiv) in DMF (5.00 mL) was added PBr3 (1.73 g, 6.41 mmol, 1.50 equiv) dropwise at 0° C. under argon atmosphere. The resulting mixture was stirred for 1 h at 0° C. under argon atmosphere. The reaction was quenched with sat. NaHCO₃ (aq.) at 0° C. The resulting mixture was extracted with EtOAc (3×20 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (5/1) to afford 8-bromo-2-cyclopropylpyrido[3,2-d]pyrimidine (650 mg, 60.82%) as a light yellow solid.

LC-MS: (M+H)⁺ found: 252.0

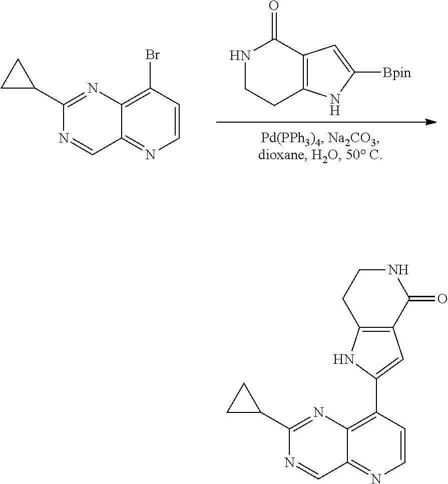

To a stirred mixture of 8-bromo-2-cyclopropylpyrido[3,2-d]pyrimidine (300 mg, 1.20 mmol, 1.00 equiv) and 2-(4,4,5,5-tetramethyl-1,3,2-dioxaborolan-2-yl)-1H,5H,6H,7H-pyrrolo[3,2-c]pyridin-4-one (377 mg, 1.44 mmol, 1.20 equiv) in 1,4-dioxane (2.00 mL) and H₂O (0.20 mL) were added Pd(PPh₃)₄ (277 mg, 0.24 mmol, 0.20 equiv) and Na2CO3 (381 mg, 3.60 mmol, 3.00 equiv) under argon atmosphere. The resulting suspension was backfilled with argon three times and stirred for overnight at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford 2-{2-cyclopropylpyrido[3,2-d]pyrimidin-8-yl}-1H,5H,6H,7H-pyrrolo[3,2-c]pyridin-4-one (110 mg, 30.03%) as a yellow solid.

LC-MS: (M+H)⁺ found: 306.

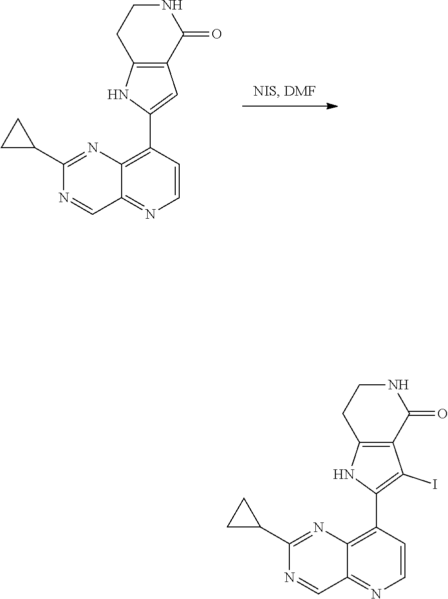 [SMILES: O=C1NCCc2[nH]c(-c3ccnc4cnc(C5CC5)nc34)cc21 ; O=C1NCCc2[nH]c(-c3ccnc4cnc(C5CC5)nc34)c(I)c21]

To a stirred solution of 2-{2-cyclopropylpyrido[3,2-d]pyrimidin-8-yl}-1H,5H,6H,7H-pyrrolo[3,2-c]pyridin-4-one (110 mg, 0.36 mmol, 1.00 equiv) in DMF (1.00 mL) was added NIS (97 mg, 0.43 mmol, 1.20 equiv) dropwise at 0° C. under argon atmosphere. The resulting mixture was stirred for 2 h at 0° C. under argon atmosphere. The reaction was quenched by the addition of sat. Na₂SO₃ (aq.) (2.00 mL) at 0° C. The resulting mixture was extracted with CH₂Cl₂/MeOH (3×10 mL). The combined organic layers were washed with brine (1×30 mL), dried over anhydrous Na₂SO₄. After filtration, the filtrate was concentrated under reduced pressure. The residue was purified by reverse flash chromatography to obtain 2-{2-cyclopropylpyrido[3,2-d]pyrimidin-8-yl}-3-iodo-1H,5H,6H,7H-pyrrolo[3,2-c]pyridin-4-one (50 mg, 32.18%) as a light-yellow solid.

LC-MS: (M+H)⁺ found: 431.85.

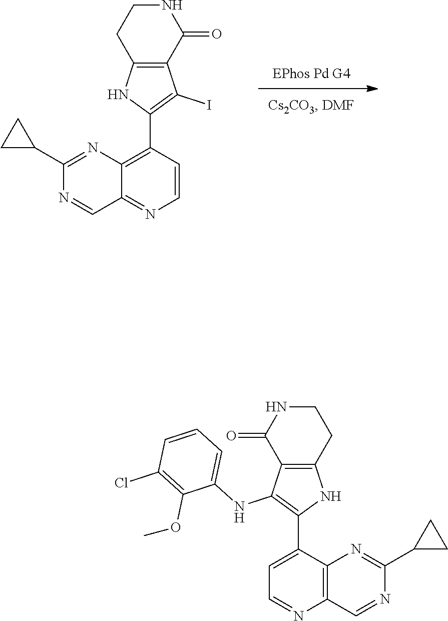 [SMILES: O=C1NCCc2[nH]c(-c3ccnc4cnc(C5CC5)nc34)c(I)c21 ; COc1c(Cl)cccc1Nc1c(-c2ccnc3cnc(C4CC4)nc23)[nH]c2c1C(=O)NCC2]

To a stirred mixture of 2-{2-cyclopropylpyrido[3,2-d]pyrimidin-8-yl}-3-iodo-1H,5H,6H,7H-pyrrolo[3,2-c]pyridin-4-one (100 mg, 0.23 mmol, 1.00 equiv) and 3-chloro-2-methoxyaniline (36 mg, 0.23 mmol, 1.00 equiv) in DMF (1.00 mL) were added EPhos Pd G4 (21 mg, 0.023 mmol, 0.10 equiv) and Cs₂CO₃ (226.66 mg, 0.69 mmol, 3.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford 3-[(3-chloro-2-methoxyphenyl)amino]-2-{2-cyclopropylpyrido[3,2-d]pyrimidin-8-yl}-1H,5H,6H,7H-pyrrolo[3,2-c]pyridin-4-one (16.3 mg, 14.49%) as a yellow solid.

LC-MS: (M+H)⁺ found: 461.25.

¹H NMR (400 MHz, DMSO-d₆) δ 12.36 (s, 1H), 9.47 (d, 1H), 8.74 (d, 1H), 7.99 (s, 1H), 7.56 (d, 1H), 7.27 (s, 1H), 6.78-6.66 (m, 2H), 6.20-6.16 (m, 1H), 4.00 (s, 3H), 3.51-3.42 (m, 2H), 3.02 (t, 2H), 2.79-2.69 (m, 1H), 1.25 (d, 4H).

### Example 166. (3R)-3′-[(3-chloro-2-methoxyphenyl)amino]-2′-(3-fluoropyridin-4-yl)-5′,6′-dihydro-1′H-spiro[oxolane-3,7′-pyrrolo[3,2-c]pyridin]-4′-one (compound 782)

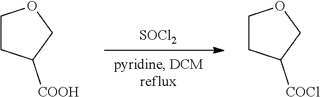 [SMILES: O=C(O)C1CCOC1 ; "SOCl2" ; O=C(Cl)C1CCOC1]

A solution of oxolane-3-carbonyl chloride (10 g, 74.32 mmol, 1.00 equiv) and SOCl₂ (9.28 g, 78.03 mmol, 1.05 equiv) and pyridine (118 mg, 1.48 mmol, 0.02 equiv) in DCM (100 mL) was stirred for 3 h at 45 degrees C. under nitrogen atmosphere. The mixture was monitored by TLC The resulting mixture was concentrated under vacuum. This resulted in oxolane-3-carbonyl chloride (11.5 g, 115.00%) as a yellow oil. The reaction is confirmed by TLC.

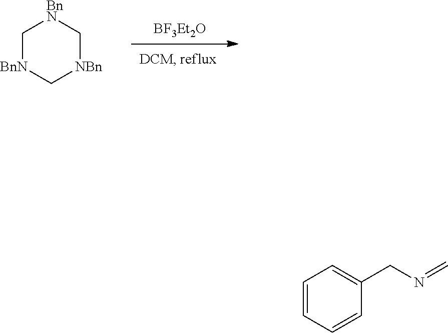

A solution of 1,3,5-tribenzyl-1,3,5-triazinane (10.3 g, 28.81 mmol, 1.00 equiv) and BF3·Et2O (12.27 g, 86.43 mmol, 3.00 equiv) in DCM (100 mL) was stirred for 3 h at 45 degrees C. under nitrogen atmosphere. The resulting mixture was concentrated under vacuum. This resulted in benzyl(methylidene)amine (11.5 g, 334.95%) as a yellow oil.

LC-MS: (M+H)⁺ found: 119.

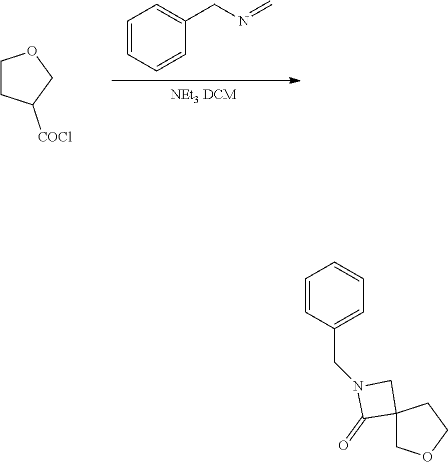

To a stirred solution of oxolane-3-carbonyl chloride (11.5 g, 85.46 mmol, 1.00 equiv) in DCM (100 mL) were added NEt3 (34.59 g, 341.86 mmol, 4.00 equiv) dropwise at −78 degrees C. under nitrogen atmosphere. The resulting mixture was stirred for 30 min at −78 degrees C. under nitrogen atmosphere. To the above mixture was added benzyl(methylidene)amine (11.20 g, 94.01 mmol, 1.1 equiv) in DCM (20 ml) dropwise over 20 min at −55 degrees C. The resulting mixture was stirred for additional 1 h at −45 degrees C. To the above mixture was added H₂O (40 ml) at −45 degrees C. The resulting mixture was stirred for overnight at room temperature. The reaction was quenched with sat. NH4Cl (aq.) at 0 degrees C. The resulting mixture was extracted with CH₂Cl₂ (2×30 mL). The combined organic layers were washed with brine (1×100 ml), dried over anhydrous Na2SO4. After filtration, the filtrate was concentrated under reduced pressure. The residue was purified by reverse flash chromatography with the following conditions: column, C18 silica gel; mobile phase, MeCN in water (NH4HCO₃, 5%), 20% to 60% gradient in 30 min; detector, UV 220 nm. This resulted in 2-benzyl-6-oxa-2-azaspiro[3.4]octan-1-one (14.7 g, 79.17%) as a yellow oil.

LC-MS: (M+H)⁺ found: 218.

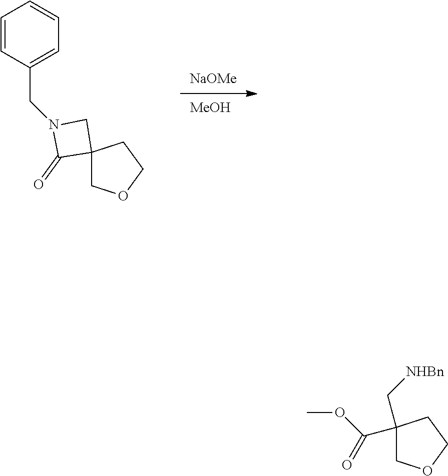

A solution of 2-benzyl-6-oxa-2-azaspiro[3.4]octan-1-one (14.7 g, 67.66 mmol, 1.00 equiv) and MeONa (4.02 g, 74.42 mmol, 1.10 equiv) in MeOH (150 mL) was stirred for overnight at 45 degrees C. under nitrogen atmosphere. The mixture was allowed to cool down to room temperature. The reaction was quenched by the addition of sat. NH4Cl (aq.) (100 mL) at room temperature. The resulting mixture was extracted with EtOAc (3×100 mL). The combined organic layers were washed with brine (1×100 mL), dried over anhydrous Na2SO4. After filtration, the filtrate was concentrated under reduced pressure. The residue was purified by reverse flash chromatography with the following conditions: column, C18 silica gel; mobile phase, MeOH in water (NH₄HCO₃, 5%), 20% to 80% gradient in 30 min; detector, UV 254 nm. This resulted in methyl 3-[(benzylamino)methyl]oxolane-3-carboxylate (12.7 g, 75.29%) as a yellow oil.

LC-MS: (M+H)⁺ found: 250.

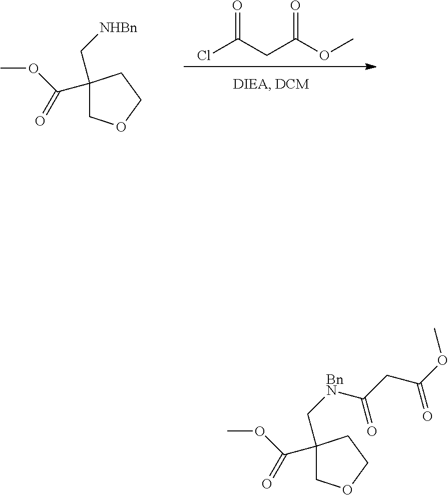

To a stirred solution of methyl 3-[(benzylamino)methyl]oxolane-3-carboxylate (14.7 g, 58.96 mmol, 1.00 equiv) in DCM (150 mL) were added DIEA (8.00 g, 61.91 mmol, 1.05 equiv) at 0 degrees C. under nitrogen atmosphere. The resulting mixture was stirred for 30 min at 0 degrees C. under nitrogen atmosphere. To the above mixture was added methyl 3-chloro-3-oxopropanoate (8.05 g, 58.96 mmol, 1 equiv) dropwise over 30 min at 0 degrees C. The resulting mixture was stirred for additional 1 h at 0 degrees C. The reaction was quenched by the addition of Water/Ice (100 mL) at 0 degrees C. The resulting mixture was extracted with CH₂Cl₂ (2×100 mL). The combined organic layers were washed with brine (1×100 ml), dried over anhydrous Na₂SO₄. After filtration, the filtrate was concentrated under reduced pressure. The residue was purified by reverse flash chromatography with the following conditions: column, C18 silica gel; mobile phase, MeOH in water (NH₄HCO₃, 5%), 20% to 80% gradient in 30 min; detector, UV 210 nm. This resulted in methyl 3-[(N-benzyl-3-methoxy-3-oxopropanamido)methyl]oxolane-3-carboxylate (18.2 g, 88.35%) as a yellow oil.

LC-MS: (M+H)⁺ found: 350.

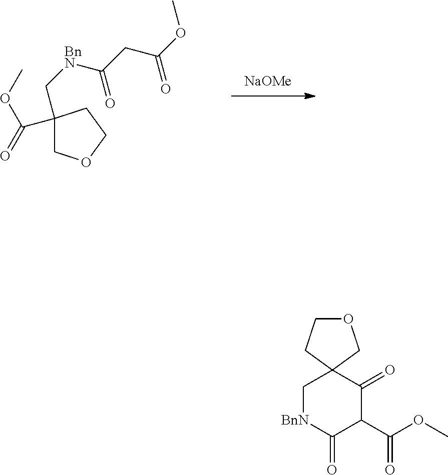

A solution/mixture of methyl 3-[(N-benzyl-3-methoxy-3-oxopropanamido)methyl]oxolane-3-carboxylate (16.7 g, 47.80 mmol, 1.00 equiv) and sodium methoxide (3.87 g, 71.70 mmol, 1.50 equiv) in toluene (160 mL) and MeOH (25 mL) was stirred for 6 h at 85 degrees C. under nitrogen atmosphere. The mixture was allowed to cool down to room temperature. The reaction was quenched by the addition of Water/Ice (100 mL) at 0 degrees C. The resulting mixture was extracted with EtOAc (3×200 mL). The combined organic layers were washed with brine (1×100 mL), dried over anhydrous Na₂SO₄. After filtration, the filtrate was concentrated under reduced pressure. This resulted in methyl 7-benzyl-8,10-dioxo-2-oxa-7-azaspiro[4.5]decane-9-carboxylate (16.5 g, 108.78%) as a yellow oil.

LC-MS: (M+H)⁺ found: 318.

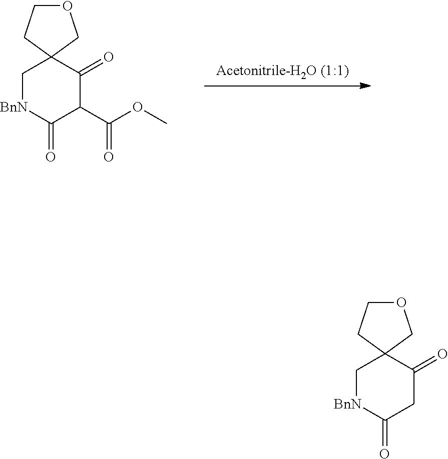 [SMILES: COC(=O)C1C(=O)N(Cc2ccccc2)CC2(CCOC2)C1=O ; O=C1CC(=O)C2(CCOC2)CN1Cc1ccccc1]

A solution of methyl 7-benzyl-8,10-dioxo-2-oxa-7-azaspiro[4.5]decane-9-carboxylate (16.5 g, 52.00 mmol, 1.00 equiv) in MeCN (100 mL) and H₂O (100 mL) was stirred for 2 h at 85 degrees C. under nitrogen atmosphere. The mixture was allowed to cool down to room temperature. The resulting mixture was extracted with EtOAc (2×200 mL). The combined organic layers were washed with brine (1×100 mL), dried over anhydrous Na₂SO₄. After filtration, the filtrate was concentrated under reduced pressure. The crude product was re-crystallized from diethyl ether (50 mL) to afford 7-benzyl-2-oxa-7-azaspiro[4.5]decane-8,10-dione (10.5 g, 77.88%) as a white solid.

LC-MS: (M+H)⁺ found: 260.

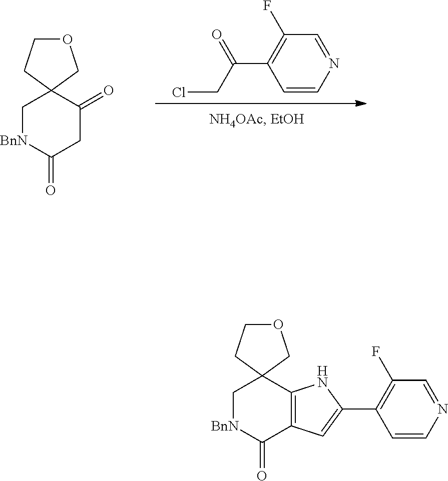 [SMILES: O=C1CC(=O)C2(CCOC2)CN1Cc1ccccc1 ; O=C(CCl)c1ccncc1F ; O=C1c2cc(-c3ccncc3F)[nH]c2C2(CCOC2)CN1Cc1ccccc1]

A solution of 7-benzyl-2-oxa-7-azaspiro[4.5]decane-8,10-dione (1.5 g, 5.79 mmol, 1 equiv) and 2-chloro-1-(3-fluoropyridin-4-yl)ethan-1-one (1.503 g, 8.59 mmol, 1.50 equiv) and NH₄OAc (2.67 g, 34.75 mmol, 6.00 equiv) in EtOH (5 mL) was stirred for overnight at 50 degrees C. under nitrogen atmosphere. The mixture was allowed to cool down to room temperature. The resulting mixture was extracted with EtOAc (2×200 mL). The combined organic layers were washed with brine (1×100 mL), dried over anhydrous Na₂SO₄. After filtration, the filtrate was concentrated under reduced pressure. The residue was purified by reverse flash chromatography with the following conditions: column, C18 silica gel; mobile phase, MeCN in water (NH₄HCO₃, 5%), 20% to 70% gradient in 30 min; detector, UV 220 nm to afford 5′-benzyl-2′-(3-fluoropyridin-4-yl)-4,5,5′,6′-tetrahydro-2H-spiro[furan-3,7′-pyrrolo[3,2-c]pyridin]-4′(1′H)-one (1 g) as a brown solid.

LC-MS: (M+H)⁺ found: 378.

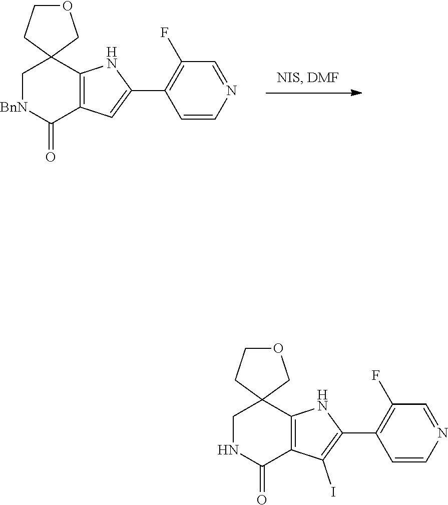 [SMILES: O=C1c2cc(-c3ccncc3F)[nH]c2C2(CCOC2)CN1Cc1ccccc1 ; O=C1NCC2(CCOC2)c2[nH]c(-c3ccncc3F)c(I)c21]

A solution of 2′-(3-fluoropyridin-4-yl)-5′,6′-dihydro-1′H-spiro[oxolane-3,7′-pyrrolo[3,2-c]pyridin]-4′-one (110 mg, 0.38 mmol, 1.00 equiv) and NIS (86 mg, 0.38 mmol, 1 equiv) in DMF (4 mL) was stirred for 2 h at room temperature under nitrogen atmosphere. The reaction was monitored by LCMS. Desired product could be detected by LCMS. The residue was purified by reverse flash chromatography with the following conditions: column, C18 silica gel; mobile phase, ACN in water, 10% to 50% gradient in 10 min; detector, UV 254 nm to afford 2′-(3-fluoropyridin-4-yl)-3′-iodo-5′,6′-dihydro-1′H-spiro[oxolane-3,7′-pyrrolo[3,2-c]pyridin]-4′-one (130 mg, 82.17%) as a yellow solid.

LC-MS: (M+H)⁺ found 414.

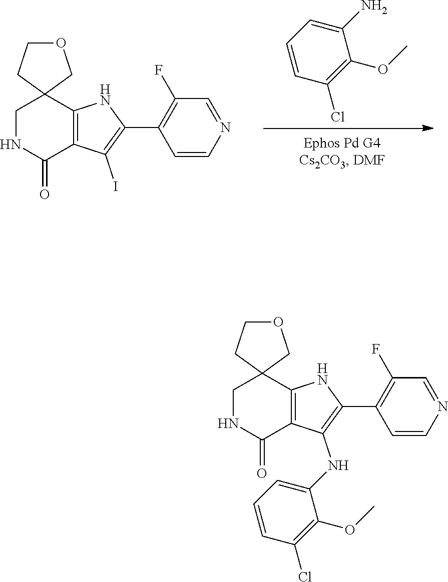

To a stirred mixture of 2′-(3-fluoropyridin-4-yl)-3′-iodo-5′,6′-dihydro-1′H-spiro[oxolane-3,7′-pyrrolo[3,2-c]pyridin]-4′-one (110 mg, 0.26 mmol, 1.00 equiv) and 3-chloro-2-methoxyaniline (42 mg, 0.26 mmol, 1.00 equiv) in DMF (2.5 mL) were added Ephos Pd G4 (24 mg, 0.03 mmol, 0.1 equiv) and Cs₂CO₃ (173 mg, 0.53 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, C18 silica gel; mobile phase, ACN in water, 10% to 50% gradient in 10 min; detector, UV 254 nm to afford 3′-[(3-chloro-2-methoxyphenyl)amino]-2′-(3-fluoropyridin-4-yl)-5′,6′-dihydro-1′H-spiro[oxolane-3,7′-pyrrolo[3,2-c]pyridin]-4′-one (95 mg, 80.57%) as a yellow solid.

LC-MS: M+H found: 443.0.

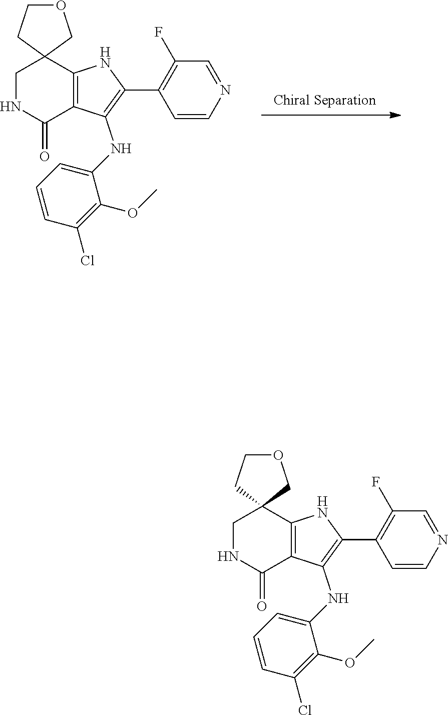 [SMILES: COc1c(Cl)cccc1Nc1c(-c2ccncc2F)[nH]c2c1C(=O)NCC21CCOC1 ; COc1c(Cl)cccc1Nc1c(-c2ccncc2F)[nH]c2c1C(=O)NC[C@]21CCOC1]

The product (95 mg) was purified by Prep-Chiral-HPLC with the following conditions (Column: CHIRALPAK IG-3, 4.6*50 mm, 3 um; Mobile Phase A: Hex:DCM=3:1) (0.1% DEA): IPA=70: 30; Flow rate: 1 mL/min; Gradient: 0% B to 0% B; Injection Volume: 5 ul mL) to afford (3R)-3′-[(3-chloro-2-methoxyphenyl)amino]-2′-(3-fluoropyridin-4-yl)-5′,6′-dihydro-1′H-spiro[oxolane-3,7′-pyrrolo[3,2-c]pyridin]-4′-one (28.8 mg, 30.10%) as a yellow solid.

¹H NMR (400 MHz, DMSO-d₆) δ 11.62 (s, 1H), 8.50 (d, J=2.5 Hz, 1H), 8.35 (m, 1H), 7.63 (s, 1H), 7.49 (m, 1H), 7.37 (m, 1H), 6.70-6.55 (m, 2H), 6.11 (m, 1H), 3.95 (m, 2H), 3.85 (d, J=7.0 Hz, 4H), 3.60 (d, J=8.6 Hz, 1H), 3.32 (s, 1H), 3.27 (m, 1H), 2.49 (m, 1H), 2.04-1.91 (m, 1H).

LC-MS: (M+H)⁺ found: 443.

### Example 167. (3R)-3′-[(3-chloro-2-methoxyphenyl)amino]-2′-(3-fluoropyridin-4-yl)-5′,6′-dihydro-1′H-spiro[oxolane-3,7′-pyrrolo[3,2-c]pyridin]-4′-one (compound 781)

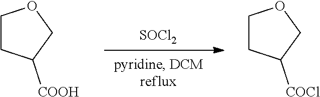 [SMILES: O=C(O)C1CCOC1 ; "SOCl2" ; O=C(Cl)C1CCOC1]

A solution of oxolane-3-carbonyl chloride (10 g, 74.32 mmol, 1.00 equiv) and SOCl₂ (9.28 g, 78.03 mmol, 1.05 equiv) and pyridine (118 mg, 1.48 mmol, 0.02 equiv) in DCM (100 mL) was stirred for 3 h at 45 degrees C. under nitrogen atmosphere. The mixture was monitored by TLC The resulting mixture was concentrated under vacuum. This resulted in oxolane-3-carbonyl chloride (11.5 g, 115.00%) as a yellow oil. The reaction is confirmed by TLC.

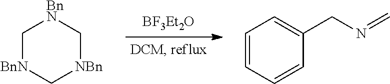

A solution of 1,3,5-tribenzyl-1,3,5-triazinane (10.3 g, 28.81 mmol, 1.00 equiv) and BF₃·Et₂O (12.27 g, 86.43 mmol, 3.00 equiv) in DCM (100 mL) was stirred for 3 h at 45 degrees C. under nitrogen atmosphere. The resulting mixture was concentrated under vacuum. This resulted in benzyl(methylidene)amine (11.5 g, 334.95%) as a yellow oil.

LC-MS: (M+H)⁺ found: 119.

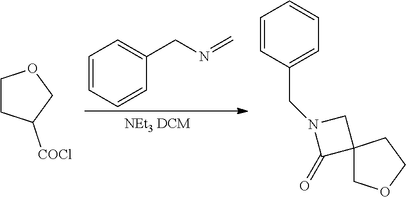

To a stirred solution of oxolane-3-carbonyl chloride (11.5 g, 85.46 mmol, 1.00 equiv) in DCM (100 mL) were added NEt₃ (34.59 g, 341.86 mmol, 4.00 equiv) dropwise at −78 degrees C. under nitrogen atmosphere. The resulting mixture was stirred for 30 min at −78 degrees C. under nitrogen atmosphere. To the above mixture was added benzyl(methylidene)amine (11.20 g, 94.01 mmol, 1.1 equiv) in DCM (20 ml) dropwise over 20 min at −55 degrees C. The resulting mixture was stirred for additional 1 h at −45 degrees C. To the above mixture was added H₂O (40 ml) at −45 degrees C. The resulting mixture was stirred overnight at room temperature. The reaction was quenched with sat. NH₄Cl (aq.) at 0 degrees C. The resulting mixture was extracted with CH₂Cl₂ (2×3 mL). The combined organic layers were washed with brine (1×100 ml), dried over anhydrous Na₂SO₄. After filtration, the filtrate was concentrated under reduced pressure. The residue was purified by reverse flash chromatography with the following conditions: column, C18 silica gel; mobile phase, MeCN in water (NH₄HCO₃, 5%), 20% to 60% gradient in 30 min; detector, UV 220 nm. This resulted in 2-benzyl-6-oxa-2-azaspiro[3.4]octan-1-one (14.7 g, 79.17%) as a yellow oil.

LC-MS: (M+H)⁺ found: 218.

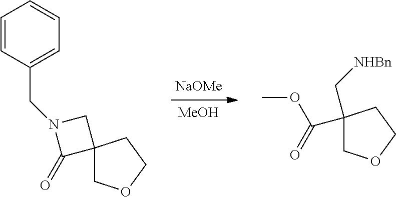

A solution of 2-benzyl-6-oxa-2-azaspiro[3.4]octan-1-one (14.7 g, 67.66 mmol, 1.00 equiv) and MeONa (4.02 g, 74.42 mmol, 1.10 equiv) in MeOH (150 mL) was stirred for overnight at 45 degrees C. under nitrogen atmosphere. The mixture was allowed to cool down to room temperature. The reaction was quenched by the addition of sat. NH₄Cl (aq.) (100 mL) at room temperature. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by reverse flash chromatography with the following conditions: column, C18 silica gel; mobile phase, MeOH in water (NH₄HCO₃, 5%), 20% to 80% gradient in 30 min; detector, UV 254 nm. This resulted in methyl 3-[(benzylamino)methyl]oxolane-3-carboxylate (12.7 g, 75.29%) as a yellow oil.

LC-MS: (M+H)⁺ found: 250.

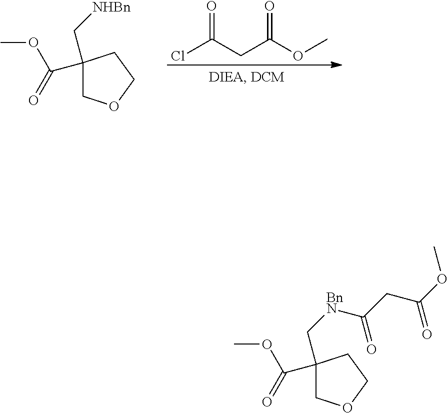

To a stirred solution of methyl 3-[(benzylamino)methyl]oxolane-3-carboxylate (14.7 g, 58.96 mmol, 1.00 equiv) in DCM (150 mL) were added DIEA (8.00 g, 61.91 mmol, 1.05 equiv) at 0 degrees C. under nitrogen atmosphere. The resulting mixture was stirred for 30 min at 0 degrees C. under nitrogen atmosphere. To the above mixture was added methyl 3-chloro-3-oxopropanoate (8.05 g, 58.96 mmol, 1 equiv) dropwise over 30 min at 0 degrees C. The resulting mixture was stirred for additional 1 h at 0 degrees C. The reaction was quenched by the addition of Water/Ice (100 mL) at 0 degrees C. The resulting mixture was extracted with CH₂Cl₂ (2×100 mL). The combined organic layers were washed with brine (1×100 ml), dried over anhydrous Na₂SO₄. After filtration, the filtrate was concentrated under reduced pressure. The residue was purified by reverse flash chromatography with the following conditions: column, C18 silica gel; mobile phase, MeOH in water (NH₄HCO₃, 5%), 20% to 80% gradient in 30 min; detector, UV 210 nm. This resulted in methyl 3-[(N-benzyl-3-methoxy-3-oxopropanamido)methyl]oxolane-3-carboxylate (18.2 g, 88.35%) as a yellow oil.

LC-MS: (M+H)⁺ found: 350.

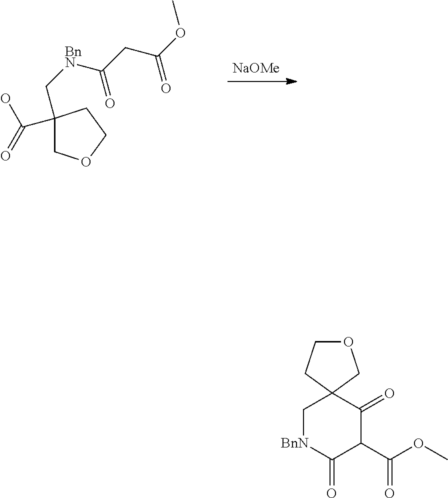

A solution/mixture of methyl 3-[(N-benzyl-3-methoxy-3-oxopropanamido)methyl]oxolane-3-carboxylate (16.7 g, 47.80 mmol, 1.00 equiv) and sodium methoxide (3.87 g, 71.70 mmol, 1.50 equiv) in toluene (160 mL) and MeOH (25 mL) was stirred for 6 h at 85 degrees C. under nitrogen atmosphere. The mixture was allowed to cool down to room temperature. The reaction was quenched by the addition of Water/Ice (I00 mL) at 0 degrees C. The resulting mixture was extracted with EtOAc (3×200 mL). The combined organic layers were washed with brine (1×100 mL), dried over anhydrous Na₂SO₄. After filtration, the filtrate was concentrated under reduced pressure. This resulted in methyl 7-benzyl-8,10-dioxo-2-oxa-7-azaspiro[4.5]decane-9-carboxylate (16.5 g, 108.78%) as a yellow oil.

LC-MS: (M+H)⁺ found: 318.

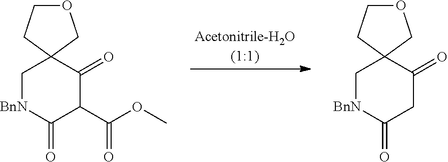 [SMILES: COC(=O)C1C(=O)N(Cc2ccccc2)CC2(CCOC2)C1=O ; "Acetonitrile-H2O(1:1)" ; O=C1CC(=O)C2(CCOC2)CN1Cc1ccccc1]

A solution of methyl 7-benzyl-8,10-dioxo-2-oxa-7-azaspiro[4.5]decane-9-carboxylate (16.5 g, 52.00 mmol, 1.00 equiv) in MeCN (100 mL) and H₂O (100 mL) was stirred for 2 h at 85 degrees C. under nitrogen atmosphere. The mixture was allowed to cool down to room temperature. The resulting mixture was extracted with EtOAc (2×200 mL). The combined organic layers were washed with brine (1×100 mL), dried over anhydrous Na₂SO₄. After filtration, the filtrate was concentrated under reduced pressure. The crude product was re-crystallized from diethyl ether (50 mL) to afford 7-benzyl-2-oxa-7-azaspiro[4.5]decane-8,10-dione (10.5 g, 77.88%) as a white solid.

LC-MS: (M+H)⁺ found: 260.

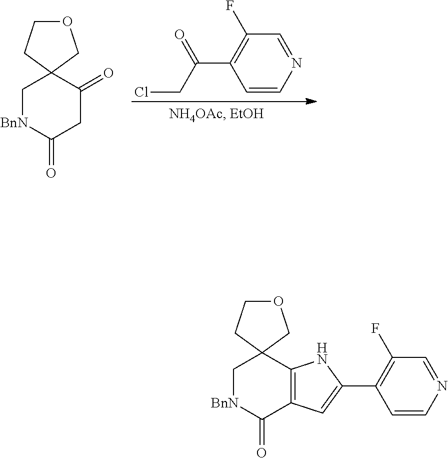

A solution of 7-benzyl-2-oxa-7-azaspiro[4.5]decane-8,10-dione (1.5 g, 5.79 mmol, 1 equiv) and 2-chloro-1-(3-fluoropyridin-4-yl)ethan-1-one (1.50 g, 8.59 mmol, 1.50 equiv) and NH₄OAc (2.67 g, 34.75 mmol, 6.00 equiv) in EtOH (5 mL) was stirred for overnight at 50 degrees C. under nitrogen atmosphere. The mixture was allowed to cool down to room temperature. The resulting mixture was extracted with EtOAc (2×200 mL). The combined organic layers were washed with brine (1×100 mL), dried over anhydrous Na₂SO₄. After filtration, the filtrate was concentrated under reduced pressure. The residue was purified by reverse flash chromatography with the following conditions: column, C18 silica gel; mobile phase, MeCN in water (NH₄HCO₃, 5%), 20% to 70% gradient in 30 min; detector, UV 220 nm to afford 5′-benzyl-2′-(3-fluoropyridin-4-yl)-4,5,5′,6′-tetrahydro-2H-spiro[furan-3,7′-pyrrolo[3,2-c]pyridin]-4′(1′H)-one (1 g) as a brown solid.

LC-MS: (M+H)⁺ found: 378.

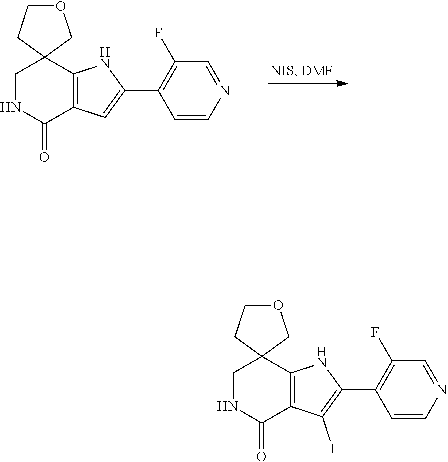 [SMILES: O=C1NCC2(CCOC2)c2[nH]c(-c3ccncc3F)cc21 ; O=C1NCC2(CCOC2)c2[nH]c(-c3ccncc3F)c(I)c21]

A solution of 2′-(3-fluoropyridin-4-yl)-5′,6′-dihydro-1′H-spiro[oxolane-3,7′-pyrrolo[3,2-c]pyridin]-4′-one (110 mg, 0.38 mmol, 1.00 equiv) and NIS (86 mg, 0.38 mmol, 1 equiv) in DMF (4 mL) was stirred for 2 h at room temperature under nitrogen atmosphere. The reaction was monitored by LCMS. Desired product could be detected by LCMS. The residue was purified by reverse flash chromatography with the following conditions: column, C18 silica gel; mobile phase, ACN in water, 10% to 50% gradient in 10 min; detector, UV 254 nm to afford 2′-(3-fluoropyridin-4-yl)-3′-iodo-5′,6′-dihydro-1′H-spiro[oxolane-3,7′-pyrrolo[3,2-c]pyridin]-4′-one (130 mg, 82.17%) as a yellow solid.

LC-MS: (M+H)⁺ found 414.

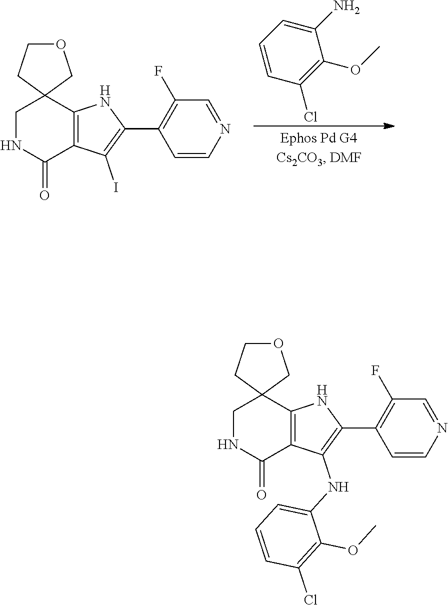

To a stirred mixture of 2′-(3-fluoropyridin-4-yl)-3′-iodo-5′,6′-dihydro-1′H-spiro[oxolane-3,7′-pyrrolo[3,2-c]pyridin]-4′-one (110 mg, 0.26 mmol, 1.00 equiv) and 3-chloro-2-methoxyaniline (42 mg, 0.26 mmol, 1.00 equiv) in DMF (2.5 mL) were added Ephos Pd G4 (24 mg, 0.03 mmol, 0.1 equiv) and Cs₂CO₃ (173 mg, 0.53 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, C18 silica gel; mobile phase, ACN in water, 10% to 50% gradient in 10 min: detector, UV 254 nm to afford 3′-[(3-chloro-2-methoxyphenyl)amino]-2′-(3-fluoropyridin-4-yl)-5′,6′-dihydro-1′H-spiro[oxolane-3,7′-pyrrolo[3,2-c]pyridin]-4′-one (95 mg, 80.57%) as a yellow solid.

LC-MS: M+H found: 443.0.

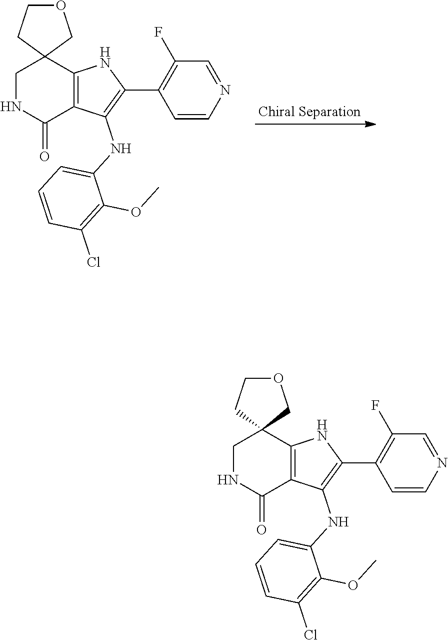 [SMILES: COc1c(Cl)cccc1Nc1c(-c2ccncc2F)[nH]c2c1C(=O)NCC21CCOC1 ; COc1c(Cl)cccc1Nc1c(-c2ccncc2F)[nH]c2c1C(=O)NC[C@]21CCOC1]

The product (95 mg) was purified by Prep-Chiral-HPLC with the following conditions (Column: CHIRALPAK IG-3, 4.6*50 mm, 3 um; Mobile Phase A: Hex:DCM=3:1) (0.1% DEA): IPA=70: 30; Flow rate: 1 mL/min; Gradient: 0% B to 0% B; Injection Volume: 5 ul mL) to afford (3R)-3′-[(3-chloro-2-methoxyphenyl)amino]-2′-(3-fluoropyridin-4-yl)-5′,6′-dihydro-1′H-spiro[oxolane-3,7′-pyrrolo[3,2-c]pyridin]-4′-one (29.5 mg, 30.87%) as a white solid.

LC-MS: (M+H)⁺ found: 443.

¹H NMR (400 MHz, DMSO-d₆) δ 11.62 (s, 1H), 8.50 (d, J=2.5 Hz, 1H), 8.35 (m, 1H), 7.63 (s, 1H), 7.49 (m, 1H), 7.37 (m, 1H), 6.70-6.55 (m, 2H), 6.11 (m, 1H), 3.95 (m, 2H), 3.85 (d, J=7.0 Hz, 4H), 3.60 (d, J=8.6 Hz, 1H), 3.32 (s, 1H), 3.27 (m, 1H), 2.49 (m, 1H), 2.04-1.91 (m, 1H).

### Example 168. 3-[(3-fluoro-2-methoxyphenyl)amino]-2-[6-[(3-methyloxetan-3-yl)oxy]-1,5-naphthyridin-4-yl]-1H,5H,6H,7H-pyrrolo[3,2-c]pyridin-4-one (compound 788)

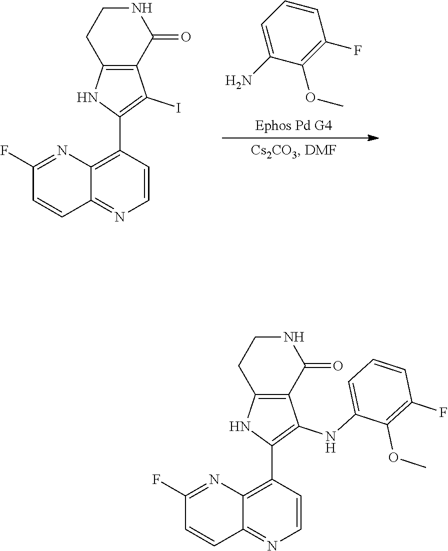

To a stirred solution of 2-(6-fluoro-1,5-naphthyridin-4-yl)-3-iodo-1H,5H,6H,7H-pyrrolo[3,2-c]pyridin-4-one (530 mg, 1.29 mmol, 1.00 equiv) in DMF (13.00 mL, 171.15 mmol, 131.86 equiv) was added Cs₂CO₃ (846 mg, 2.59 mmol, 2.00 equiv) and EPhos Pd G4 (119 mg, 0.13 mmol, 0.10 equiv) in portions at room temperature under nitrogen atmosphere. To the above mixture was added 3-fluoro-2-methoxyaniline (201 mg, 1.42 mmol, 1.10 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford 2-(6-fluoro-1,5-naphthyridin-4-yl)-3-[(3-fluoro-2-methoxyphenyl)amino]-1H,5H,6H,7H-pyrrolo[3,2-c]pyridin-4-one (320 mg, 58.48%) as a yellow solid.

LC-MS: (M+H)⁺ found: 422.1.

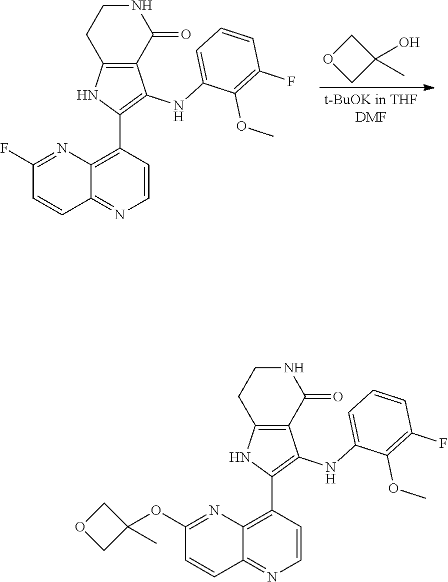

To a stirred solution of 2-(6-fluoro-1,5-naphthyridin-4-yl)-3-[(3-fluoro-2-methoxyphenyl)amino]-1H,5H,6H,7H-pyrrolo[3,2-c]pyridin-4-one (120 mg, 0.28 mmol, 1.00 equiv) in DMF (3.00 mL) were added 3-methyloxetan-3-ol (75 mg, 0.85 mmol, 3.00 equiv) dropwise at room temperature under nitrogen atmosphere. To the above mixture was added t-BuOK (35 mg, 0.31 mmol, 1.10 equiv) dropwise at 0 degrees C. The resulting mixture was stirred for additional 24 h at room temperature. The reaction was monitored by LCMS. LCMS showed the reaction was completed. The resulting mixture was filtered, The filtrate was concentrated under reduced pressure. The crude product was purified by Prep-HPLC with the following conditions (Column. Xselect CSH OBD Column 30*150 mm Sum, n; Mobile Phase A: Water (0.1% FA), Mobile Phase B: ACN; Flow rate: 60 mL/min; Gradient: 24% B to 42% B in 8 min, 42% B; Wave Length: 254/220 nm; RT1 (min): 7.33) to afford 3-[(3-fluoro-2-methoxyphenyl)amino]-2-[6-[(3-methyloxetan-3-yl)oxy]-1,5-naphthyridin-4-yl]-1H,5H,6H,7H-pyrrolo[3,2-c]pyridin-4-one (16.1 mg, 11.55%) as a yellow solid.

LC-MS: (M+H)⁺ found: 490.3.

¹H NMR (400 MHz, DMSO-d6) δ 11.45 (s, 1H), 8.61 (d, J=4.0 Hz, 1H), 8.33 (d, J=8.0 Hz, 1H), 8.17 (s, 1H), 7.53 (d, J=4.0 Hz, 1H), 7.36-7.32 (m, 2H), 7.15 (s, 1H), 6.53-6.36 (m, 2H), 5.98 (d, J=8.0 Hz, 1H), 4.95 (d, J=8.0 Hz, 2H), 4.48 (d, J=4.0 Hz, 2H), 3.87 (s, 3H), 3.46-3.45 (m, 2H), 2.89 (t, J=8.0 Hz, 2H), 1.86 (s, 3H).

### Example 169. 3-[(3-chloro-2-methoxyphenyl) amino]-2-[6-[(3-methyloxetan-3-yl) oxy]-1,5-naphthyridin-4-yl]-1H,5H,6H,7H-pyrrolo[3,2-c] pyridin-4-one (compound 787)

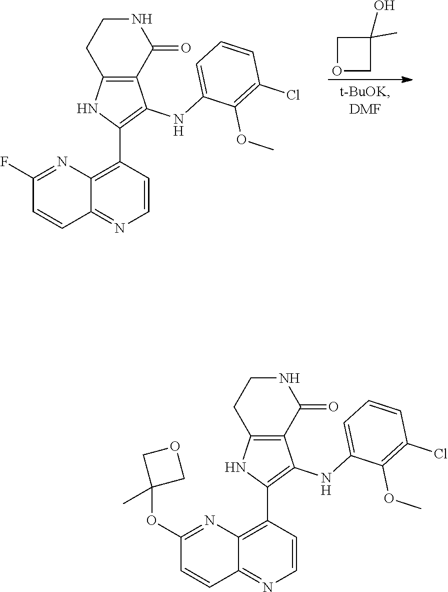

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (80 mg, 0.18 mmol, 1.00 equiv) and 3-methyloxetan-3-ol (80 mg, 0.91 mmol, 5.00 equiv) in DMF (1.00 mL) was added t-BuOK (1M in THF) (0.20 mL, 0.20 mmol, 1.10 equiv) dropwise at 0 degrees C. under nitrogen atmosphere. The resulting mixture was stirred for overnight at 50 degrees C. under nitrogen atmosphere. Desired product could be detected by LCMS. The crude product was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 27% B to 47% B in 8 min, 47% B; Wave Length. 254/220 nm; RT1 (min): 7.43) to afford 3-[(3-chloro-2-methoxyphenyl) amino]-2-[6-[(3-methyloxetan-3-yl) oxy]-1,5-naphthyridin-4-yl]-1H,5H,6H,7H-pyrrolo[3,2-c] pyridin-4-one (13.0 mg, 12.66%) as an orange solid.

LC-MS: (M+H)⁺ found: 506.00.

¹H NMR (400 MHz, DMSO-d6) δ 11.47 (s, 1H), 8.64 (d, J=4.6 Hz, 1H), 8.34 (d, J=9.1 Hz, 1H), 7.56 (d, J=4.6 Hz, 1H), 7.46-7.30 (m, 2H), 7.17 (t, J=2.6 Hz, 1H), 6.65-6.48 (m, 2H), 6.18-6.09 (m, 1H), 4.96 (d, J=7.0 Hz, 2H), 4.50 (d, J=7.0 Hz, 2H), 3.86 (s, 3H), 3.52-3.42 (m, 2H), 2.90 (t, J=6.8 Hz, 2H), 1.87 (s, 3H).

### Example 170. 3-{[2-(2,2-difluoroethyl)-3-fluorophenyl]amino}-2-{2-methoxypyrido[3,2-d]pyrimidin-8-yl}-1H,5H,6H,7H-pyrrolo[3,2-c]pyridin-4-one (compound 795)

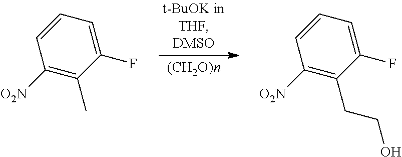

To a stirred mixture of 1-fluoro-2-methyl-3-nitrobenzene (10.00 g, 64.463 mmol, 1.00 equiv) and DMSO (100.00 mL) were added t-BuOK (1.16 g, 10.314 mmol, 0.16 equiv) and methoxymethanol amine (2.84 g, 64.463 mmol, 1.00 equiv) in portions at room temperature under nitrogen atmosphere. The resulting mixture was stirred for 1 h at 70 degrees C. under nitrogen atmosphere. The reaction was monitored by TLC. The mixture was allowed to cool down to room temperature. The mixture was acidified to pH 7 with HCl (aq.). The resulting mixture was diluted with water (200 mL). The aqueous layer was extracted with EtOAc (3×200 mL). The resulting mixture was washed with 3×200 mL of brine. The resulting mixture was concentrated under reduced pressure. The residue was purified by silica gel column chromatography, eluted with PE/EA (10:1) to afford 2-(2-fluoro-6-nitrophenyl)ethanol (8.3 g, 69.54%) as a yellow oil. H-NMR analysis indicated it was the desired product.

¹H NMR (400 MHz, Chloroform-d) δ 7.73 (dt, J=7.8, 1.4 Hz, 1H), 7.45-7.25 (m, 2H), 3.94 (t, J=6.6 Hz, 2H), 3.22 (td, J=6.6, 2.2 Hz, 2H).

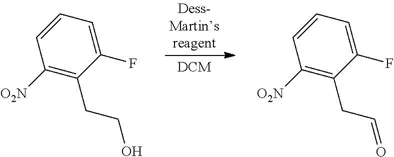 [SMILES: O=[N+]([O-])c1cccc(F)c1CCO ; O=CCc1c(F)cccc1[N+](=O)[O-]]

To a stirred mixture of 2-(2-fluoro-6-nitrophenyl)ethanol (8.30 g, 44.828 mmol, 1.00 equiv) in DCM (88.00 mL) was added Dess-Martin (19.96 g, 47.069 mmol, 1.05 equiv) in portions at room temperature under nitrogen atmosphere. The resulting mixture was stirred for overnight at room temperature under nitrogen atmosphere. The reaction was monitored by TLC. The reaction was quenched with Na2CO3 (aq.) at 0 degrees C. The aqueous layer was extracted with CH₂Cl₂ (3×100 mL). The resulting mixture was washed with 3×100 mL of brine. The resulting mixture was concentrated under reduced pressure. The residue was purified by silica gel column chromatography, eluted with PE/EA (10:1) to afford 2-(2-fluoro-6-nitrophenyl)acetaldehyde (5.10 g, 62.12%) as a yellow oil. H-NMR analysis indicated it was the desired product.

1H NMR (300 MHz, DMSO-d6) δ 9.75 (d, J=1.1 Hz, 1H), 7.97 (dt, J=8.0, 1.3 Hz, 1H), 7.81-7.47 (m, 2H), 4.20 (d, J=1.5 Hz, 2H).

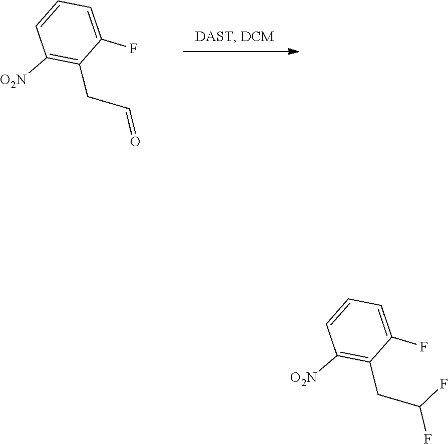 [SMILES: O=CCCc1c(F)cccc1[N+](=O)[O-] ; O=[N+]([O-])c1cccc(F)c1CCC(F)F]

To a stirred mixture of 2-(2-fluoro-6-nitrophenyl)acetaldehyde (3.00 g, 16.381 mmol, 1.00 equiv) and DCM (40.00 mL) was added DAST (6.60 g, 40.953 mmol, 2.50 equiv) in portions at −30 degrees C. under nitrogen atmosphere. The resulting mixture was stirred for overnight at room temperature under nitrogen atmosphere. The reaction was monitored by TLC. The mixture was allowed to cool down to −30 degrees C. The reaction was quenched by the addition of MeOH (30 mL). The mixture was neutralized to pH 8 with saturated Na2CO3 (aq.). The resulting mixture was diluted with water (200 mL). The resulting mixture was extracted with EtOAc (3×200 mL)). The combined organic layers were washed with brine (3×200 mL), dried over anhydrous Na2SO4. After filtration, the filtrate was concentrated under reduced pressure. The residue was purified by silica gel column chromatography, eluted with PE/EA (10:1) to afford 2-(2,2-difluoroethyl)-1-fluoro-3-nitrobenzene (300 mg, 8.93%) as a yellow oil. H-NMR analysis indicated it was the desired product.

1H NMR (300 MHz, DMSO-d6) δ 7.95 (dt, J=7.9, 1.4 Hz, 1H), 7.80-7.58 (m, 2H), 6.65-6.05 (m, 1H), 3.56 (tdd, J=17.7, 4.0, 2.0 Hz, 2H).

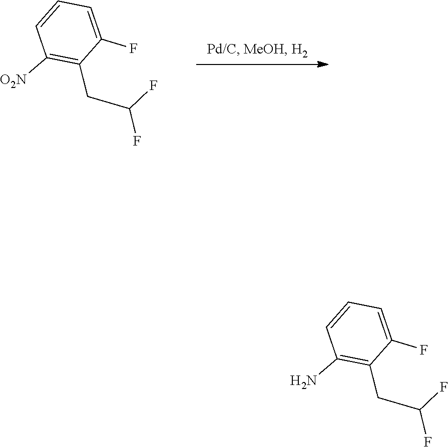 [SMILES: O=[N+]([O-])c1cccc(F)c1CCC(F)F ; Nc1cccc(F)c1CCC(F)F]

To a stirred mixture of 2-(2,2-difluoroethyl)-1-fluoro-3-nitrobenzene (300.00 mg, 1.462 mmol, 1.00 equiv) in MeOH (20.00 mL) were added Pd/C (311.27 mg, 0.292 mmol, 0.20 equiv, 10%) in portions at room temperature under nitrogen atmosphere. The resulting mixture was stirred for 5 h at room temperature under hydrogen atmosphere. The reaction was monitored by LCMS. Desired product could be detected by LCMS. The resulting mixture was filtered, the filter cake was washed with MeOH (3×50 mL). The filtrate was concentrated under reduced pressure to afford 2-(2,2-difluoroethyl)-3-fluoroaniline (180.00 mg, 59.59%) as a yellow oil. The crude product was used in the next step directly without further purification.

LC-MS: M+H found: 176.0.

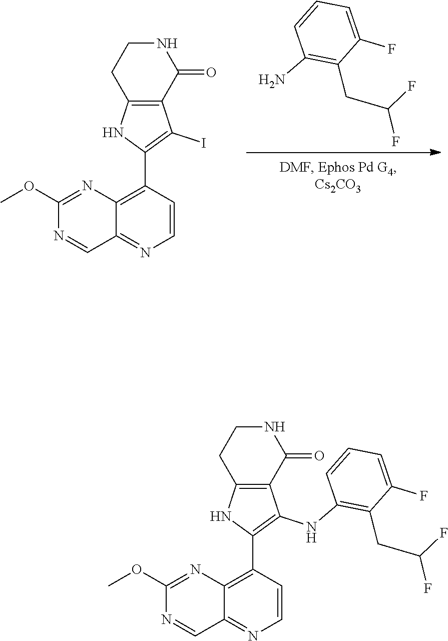 [SMILES: COc1ncc2nccc(-c3[nH]c4c(c3I)C(=O)NCC4)c2n1 ; Nc1cccc(F)c1CCC(F)F ; COc1ncc2nccc(-c3[nH]c4c(c3Nc3cccc(F)c3CCC(F)F)C(=O)NCC4)c2n1]

To a stirred mixture of 3-iodo-2-{2-methoxypyrido[3,2-d]pyrimidin-8-yl}-1H,5H,6H,7H-pyrrolo[3,2-c]pyridin-4-one (80.00 mg, 0.190 mmol, 1.00 equiv) and 2-(2,2-difluoroethyl)-3-fluoroaniline (49.90 mg, 0.285 mmol, 1.50 equiv) in DMF (2.00 ml) were added EPhos Pd G4 (34.89 mg, 0.038 mmol, 0.20 equiv) and Cs₂CO₃ (123.77 mg, 0.380 mmol, 2.00 equiv) in portions at room temperature under argon atmosphere. The resulting mixture was stirred for 3 h at 50 degrees C. under argon atmosphere. The reaction was monitored by LCMS. Desired product could be detected by LCMS. The resulting mixture was concentrated under reduced pressure. The residue was purified by silica gel column chromatography, eluted with CH₂Cl₂/MeOH (10:1) to afford 3-{[2-(2,2-difluoroethyl)-3-fluorophenyl]amino}-2-{2-methoxypyrido[3,2-d]pyrimidin-8-yl}-1H,5H,6H,7H-pyrrolo[3,2-c]pyridin-4-one (80 mg) as a yellow solid. The crude product (80 mg) was purified by Prep-HPLC with the following conditions (Column: XSelect CSH Fluoro Phenyl, 30*150 mm, 5 μm; Mobile Phase A: Water (0.05% TFA), Mobile Phase B: ACN; Flow rate: 60 mL/min; Gradient: 33% B to 49% B in 8 min, 49% B; Wave Length: 254/220 nm; RT1 (min): 7.85) to afford 3-{[2-(2,2-difluoroethyl)-3-fluorophenyl]amino}-2-{2-methoxypyrido[3,2-d]pyrimidin-8-yl}-1H,5H,6H,7H-pyrrolo[3,2-c]pyridin-4-one (12.4 mg, 13.74%) as a yellow solid.

LC-MS: (M+H)⁺ found: 469.0.

¹H NMR (300 MHz, DMSO-d₆) δ 12.05 (s, 1H), 9.49 (s, 1H), 8.65 (d, J=4.7 Hz, 1H), 7.69 (d, J=4.7 Hz, 2H), 7.23 (d, J=2.5 Hz, 1H), 6.89 (q, J=7.8 Hz, 1H), 6.76-6.38 (m, 2H), 6.18 (d, J=8.2 Hz, 1H), 4.21 (s, 3H), 3.52-3.40 (m, 4H), 2.98 (t, J=6.8 Hz, 2H).

### Example 171. 3-[(3-fluoro-2-methylphenyl)amino]-2-[2-methoxypyrido[3,2-d]pyrimidin-8-yl]-1H,5H,6H,7H-pyrrolo[3,2-c]pyridin-4-one (compound 794)

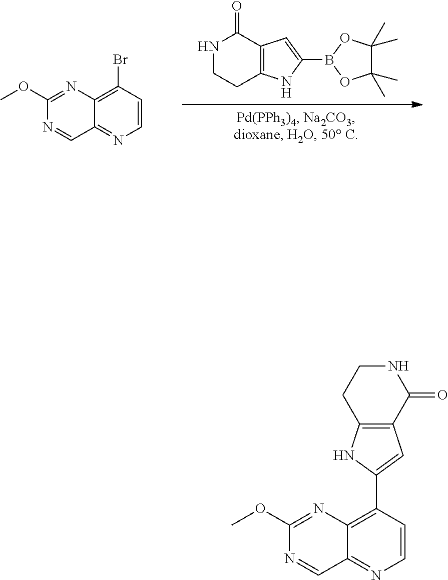 [SMILES: COc1ncc2nccc(Br)c2n1 ; CC1(C)OB(c2cc3c([nH]2)CCNC3=O)OC1(C)C ; COc1ncc2nccc(-c3cc4c([nH]3)CCNC4=O)c2n1]

To a stirred mixture of 8-bromo-2-methoxypyrido[3,2-d]pyrimidine (500 mg, 2.08 mmol, 1.00 equiv) and 2-(4,4,5,5-tetramethyl-1,3,2-dioxaborolan-2-yl)-1H,5H,6H,7H-pyrrolo[3,2-c]pyridin-4-one (655 mg, 2.50 mmol, 1.20 equiv) in dioxane (15.00 mL, 177.06 mmol, 85.01 equiv) was added Na₂CO₃ (662 mg, 6.24 mmol, 3.00 equiv) and Pd(PPh₃)₄ (240 mg, 0.20 mmol, 0.10 equiv) in portions at room temperature under nitrogen atmosphere. To the above mixture was added H₂O (3.00 mL) at room temperature. The resulting mixture was stirred for additional overnight at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford 2-{2-methoxypyrido[3,2-d]pyrimidin-8-yl}-1H,5H,6H,7H-pyrrolo[3,2-c]pyridin-4-one (410 mg, 66.66%) as a yellow solid.

LC-MS: (M+H)⁺ found: 295.95.

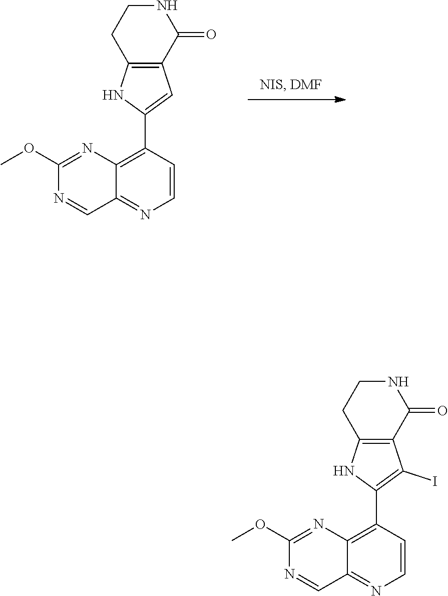 [SMILES: COc1ncc2nccc(-c3cc4c([nH]3)CCNC4=O)c2n1 ; COc1ncc2nccc(-c3[nH]c4c(c3I)C(=O)NCC4)c2n1]

To a stirred solution of 2-[2-methoxypyrido[3,2-d]pyrimidin-8-yl]-1H,5H,6H,7H-pyrrolo[3,2-c]pyridin-4-one (720 mg, 2.43 mmol, 1.00 equiv) in DMF (25.00 mL) was added N-iodosuccinimide (603 mg, 2.68 mmol, 1.10 equiv) in portions at 0 degrees C. under nitrogen atmosphere. The resulting mixture was stirred for overnight at room temperature under nitrogen atmosphere. The reaction was monitored by LCMS. LCMS showed the reaction was completed. The reaction was quenched by the addition of sat. Na₂SO₃ (aq.) (10 mL) at 0 degrees C. The resulting mixture was concentrated under reduced pressure. The residue was purified by silica gel column chromatography, eluted with CH₂C₂/MeOH (10:1) to afford 3-iodo-2-[2-methoxypyrido[3,2-d]pyrimidin-8-yl]-1H,5H,6H,7H-pyrrolo[3,2-c]pyridin-4-one (1 g, 97.38%) as a yellow solid.

LC-MS: (M+H)⁺ found 421.95.

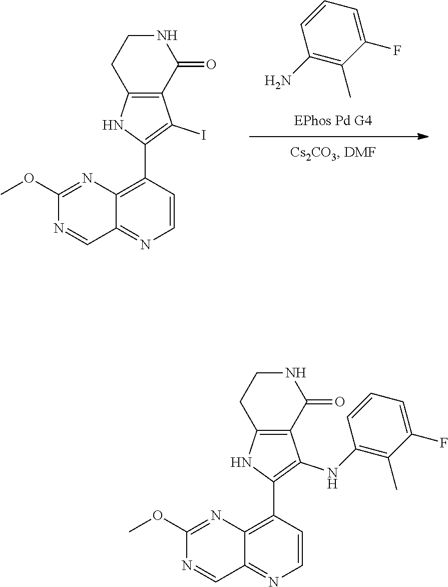

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (174 mg, 0.19 mmol, 0.10 equiv) at room temperature under nitrogen atmosphere. To the above mixture was added 3-fluoro-2-methyl-aniline (71 mg, 0.57 mmol, 3.00 equiv) at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep C18 OBD Column, 30*100 mm, 5|Ìm; Mobile Phase A: Water (10 mmol/L NH4HCO3), Mobile Phase B: ACN; Flow rate: 60 mL/min; Gradient: 26% B to 50% B in 9 min, 50% B; Wave Length: 254/220 nm; RT1 (min): 8.85) to afford 3-[(3-fluoro-2-methylphenyl)amino]-2-[2-methoxypyrido[3,2-d]pyrimidin-8-yl]-1H,5H,6H,7H-pyrrolo[3,2-c]pyridin-4-one (18.5 mg, 22.11%) as a yellow solid.

LC-MS: (M+H)⁺ found: 419.25.

¹H NMR (400 MHz, DMSO-d6) δ 12.01 (s, 1H), 9.47 (s, 1H), 8.62 (d, J=8.0 Hz, 1H), 7.66 (s, 1H), 7.45 (d, J=4.0 Hz, 1H), 7.30 (s, 1H), 6.76-6.74 (m, 1H), 6.51 (t, J=8.0 Hz, 1H), 6.07 (d, J=8.0 Hz, 1H), 4.21 (s, 3H), 3.48-3.44 (m, 2H), 2.97 (t, J=8.0 Hz, 2H), 2.23 (s, 3H).

### Example 172. 3-[(3-chloro-2-methylphenyl)amino]-2-[2-methoxypyrido[3,2-d]pyrimidin-8-yl]-1H,5H,6H,7H-pyrrolo[3,2-c]pyridin-4-one (compound 793)

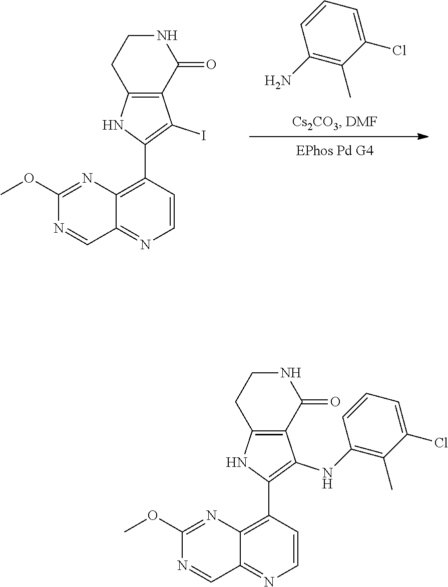

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 2-methyl-3-chloroaniline (26 mg, 0.19 mmol, 1.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep C18 OBD Column, 30*100 mm, 5|Ìm; Mobile Phase A: Water (10 mmol/L NH4HCO3), Mobile Phase B: ACN; Flow rate: 60 mL/min; Gradient: 31% B to 56% B in 9 min, 56% B; Wave Length: 254/220 nm; RT1 (min): 7.53) to afford 3-[(3-chloro-2-methylphenyl)amino]-2-[2-methoxypyrido[3,2-d]pyrimidin-8-yl]-1H,5H,6H,7H-pyrrolo[3,2-c]pyridin-4-one (3.3 mg, 3.98%) as a yellow solid.

LC-MS: (M+H)⁺ found: 434.95.

¹H NMR (400 MHz, DMSO-d6) δ 12.01 (s, 1H), 9.47 (s, 1H), 8.63 (d, J=8.0 Hz, 1H), 7.69 (s, 1H), 7.46 (d, J=4.0 Hz, 1H), 7.29 (s, 1H), 6.79-6.73 (m, 2H), 6.22-6.20 (m, 1H), 4.21 (s, 3H), 3.46 (s, 2H), 2.98 (t, J=6.8 Hz, 2H), 2.40-2.32 (m, 3H).

### Example 173. 3-[(3-chloro-5-fluoro-2-methoxyphenyl)amino]-2-[2-methoxypyrido[3,2-d]pyrimidin-8-yl]-1H,5H,6H,7H-pyrrolo[3,2-c]pyridin-4-one (compound 792)

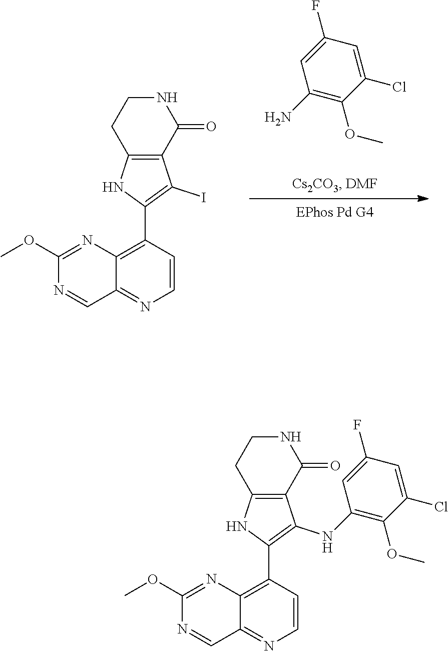 [SMILES: COc1ncc2nccc(-c3[nH]c4c(c3I)C(=O)NCC4)c2n1 ; COc1c(N)cc(F)cc1Cl ; COc1ncc2nccc(-c3[nH]c4c(c3Nc3cc(F)cc(Cl)c3OC)C(=O)NCC4)c2n1]

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 3-chloro-5-fluoro-2-methoxyaniline (33 mg, 0.19 mmol, 1.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep OBD C18 Column, 30*150 mm, 5|Ìm; Mobile Phase A: Water (10 mmol/L NH4HCO3), Mobile Phase B: ACN; Flow rate: 60 mL/min; Gradient: 31% B to 61% B in 7 min; Wave Length: 254 nm; RT1 (min): 6.5) to afford 3-[(3-chloro-5-fluoro-2-methoxyphenyl)amino]-2-[2-methoxypyrido[3,2-d]pyrimidin-8-yl]-1H,5H,6H,7H-pyrrolo[3,2-c]pyridin-4-one (16.7 mg, 18.75%) as a yellow solid.

LC-MS: (M+H)⁺ found: 468.95.

¹H NMR (400 MHz, DMSO-d6) δ 12.03 (s, 1H), 9.52 (s, 1H), 8.78 (d, J=4.0 Hz, 1H), 7.99 (s, 1H), 7.7 (d, J=4.0 Hz, 1H), 7.21 (s, 1H), 6.60-6.57 (m, 1H), 5.93-5.89 (m, 1H), 4.19 (s, 3H), 3.80 (s, 3H), 3.48-3.44 (m, 2H), 2.96 (t, J=8.0 Hz, 2H).

### Example 174. 3-[(2-ethyl-3-fluorophenyl)amino]-2-[2-methoxypyrido[3,2-d]pyrimidin-8-yl]-1H,5H,6H,7H-pyrrolo[3,2-c]pyridin-4-one (compound 791)

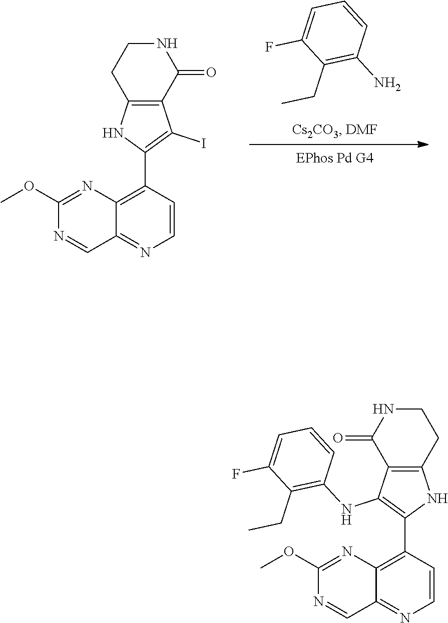

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 2-ethyl-3-fluoroaniline (79 mg, 0.57 mmol, 3.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The crude product was purified by Prep-HPLC with the following conditions (Column: XBridge Prep OBD C18 Column, 30*150 mm, 5|Ìm; Mobile Phase A: Water (10 mmol/L NH₄HCO3), Mobile Phase B: ACN; Flow rate: 60 mL/min; Gradient: 32% B to 62% B in 7 min; Wave Length: 254 nm; RT1 (min): 6.5) to afford 3-[(2-ethyl-3-fluorophenyl)amino]-2-[2-methoxypyrido[3,2-d]pyrimidin-8-yl]-1H,5H,6H,7H-pyrrolo[3,2-c]pyridin-4-one (8.7 mg, 10.59%) as a yellow solid.

LC-MS: (M+H)⁺ found: 433.

¹H NMR (400 MHz, DMSO-d6) δ 12.02 (s, 1H), 9.47 (s, 1H), 8.60 (d, J=8.0 Hz, 1H), 7.76 (s, 1H), 7.41 (d, J=4.0 Hz, 1H), 7.29 (s, 1H), 6.77-6.72 (1H), 6.54-6.49 (m, 1H), 6.11 (d, J=8.0 Hz, 1H), 4.22 (s, 3H), 3.49-3.45 (m, 2H), 2.97 (t, J=8.0 Hz, 2H), 2.75-2.67 (m, 2H), 1.26 (s, 3H).

### Example 175. 3-[(3-chloro-2-ethylphenyl)amino]-2-[2-methoxypyrido[3,2-d]pyrimidin-8-yl]-1H,5H,6H,7H-pyrrolo[3,2-c]pyridin-4-one (compound 789)

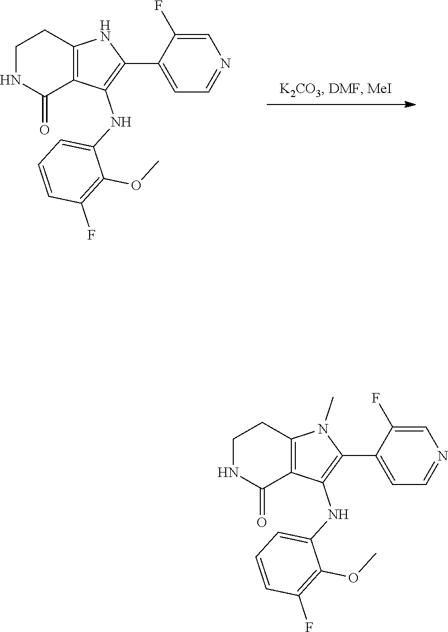 [SMILES: COc1c(F)cccc1Nc1c(-c2ccncc2F)[nH]c2c1C(=O)NCC2 ; COc1c(F)cccc1Nc1c2c(n(C)c1-c1ccncc1F)CCNC2=O]

To a stirred solution of 3-[(3-fluoro-2-methoxyphenyl)amino]-2-(3-fluoropyridin-4-yl)-1H,5H,6H,7H-pyrrolo[3,2-c]pyridin-4-one (50 mg, 0.13 mmol, 1.00 equiv) and MeI (19 mg, 0.13 mmol, 1 equiv) in DMF (2 mL) was added K₂CO₃ (37 mg, 0.27 mmol, 2 equiv) at room temperature under nitrogen atmosphere. The resulting mixture was stirred overnight at room temperature under nitrogen atmosphere. The resulting mixture was diluted with hexane (20 mL). The aqueous layer was extracted with EtOAc (3×10 mL). The crude product (30.00 mg) was purified by Prep-HPLC with the following conditions (Column: Xselect CSH OBD Column 30*150 mm Sum, n: Mobile Phase A: Water (10 mmol/L NH4HCO3), Mobile Phase B: ACN; Flow rate: 60 mL/min; Gradient: 23% B to 38% B in 9.4 min, 38% B; Wave Length: 220/254 nm; RT1 (min): 9.40) to afford 3-[(3-fluoro-2-methoxyphenyl)amino]-2-(3-fluoropyridin-4-yl)-1-methyl-5H,6H,7H-pyrrolo[3,2-c]pyridin-4-one (10.3 mg, 19.72%) as a white solid

LC-MS: (M+H)⁺ found: 385.00.

¹H NMR (400 MHz, DMSO-6) δ 8.59 (d, J=1.9 Hz, 1H), 8.40 (dd, J=4.9, 1.2 Hz, 1H), 7.47 (dd, J=6.5, 4.9 Hz, 1H), 7.13 (d, J=2.5 Hz, 2H), 6.55 (td, J=8.3, 6.1 Hz, 1H), 6.39 (ddd, J=10.8, 8.3, 1.5 Hz, 1H), 5.96 (dt, J=8.3, 1.3 Hz, 1H), 3.80 (d, J=0.7 Hz, 3H), 3.46 (dd, J=6.5, 2.1 Hz, 5H), 2.90 (t, J=6.8 Hz, 2H).

### Example 176. 3-[(3-chloro-2-ethylphenyl)amino]-2-[2-methoxypyrido[3,2-d]pyrimidin-8-yl]-1H,5H,6H,7H-pyrrolo[3,2-c]pyridin-4-one (compound 800)

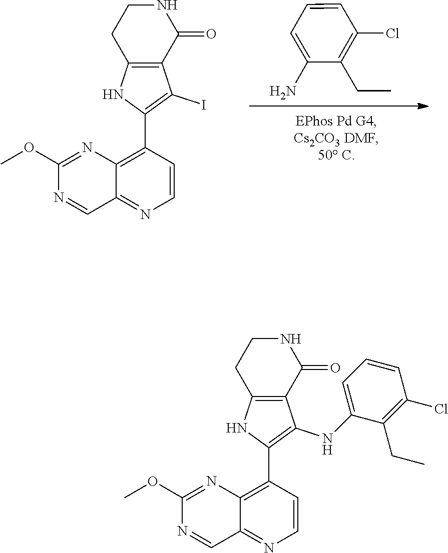

To a stirred mixture of 3-iodo-2-[2-methoxypyrido[3,2-d]pyrimidin-8-yl]-1H,5H,6H,7H-pyrrolo[3,2-c]pyridin-4-one (80 mg, 0.190 mmol, 1.00 equiv) and 3-chloro-2-ethylaniline (29 mg, 0.19 mmol, 1.00 equiv) and EPhos Pd G4 (17 mg, 0.019 mmol, 0.1 equiv) and Cs₂CO₃ (123 mg, 0.38 mmol, 2 equiv) in DMF (1.90 mL) in portions at room temperature under argon atmosphere. The resulting mixture was stirred for 2 h at 50 degrees C. under argon atmosphere. Desired product could be detected by LCMS. The crude product (80 mg) was purified by Prep-HPLC with the following conditions (Column: XBridge Prep C18 OBD Column, 30*100 mm, 5|Ìm; Mobile Phase A: Water (10 mmol/L NH4HCO3), Mobile Phase B: ACN; Flow rate: 60 m/min; Gradient: 36% B to 55% B in 10 min, 55% B; Wave Length: 254/220 nm; RT1 (min): 8.85) to afford 3-[(3-chloro-2-ethylphenyl)amino]-2-[2-methoxypyrido[3,2-d]pyrimidin-8-yl]-1H,5H,6H,7H-pyrrolo[3,2-c]pyridin-4-one (9.2 mg, 10.67%) as a yellow solid.

LC-MS: (M+H)⁺ found: 449.

¹H NMR (400 MHz, DMSO-d6) δ 12.03 (s, 1H), 9.48 (s, 1H), 8.62 (d, J=4.8 Hz, 1H), 7.79 (s, 1H), 7.41 (d, J=4.8 Hz, 1H), 7.31 (d, J=2.6 Hz, 1H), 6.89-6.63 (m, 2H), 6.25 (dd, J=7.6, 1.7 Hz, 1H), 4.22 (s, 3H), 3.48 (td, J=6.9, 2.5 Hz, 2H), 2.99 (t, J=6.8 Hz, 2H), 2.91 (q, J=7.4 Hz, 2H), 1.27 (t, J=7.5 Hz, 3H).

### Example 177. 3-[(3-chloro-2-fluorophenyl)amino]-2-[2-methoxypyrido[3,2-d]pyrimidin-8-yl]-1H,5H,6H,7H-pyrrolo[3,2-c]pyridin-4-one (compound 799)

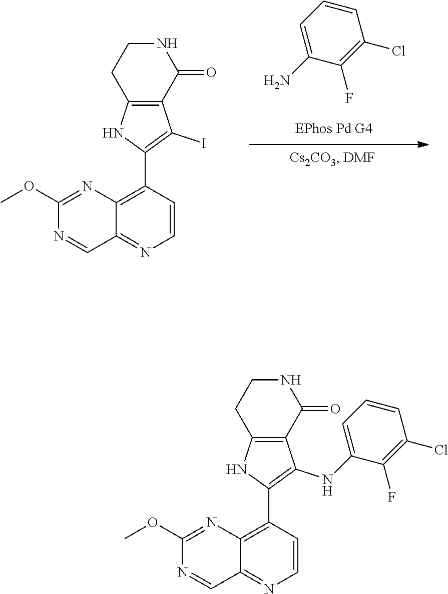 [SMILES: COc1ncc2nccc(-c3[nH]c4c(c3I)C(=O)NCC4)c2n1 ; Nc1cccc(Cl)c1F ; COc1ncc2nccc(-c3[nH]c4c(c3Nc3cccc(Cl)c3F)C(=O)NCC4)c2n1]

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 3-chloro-2-fluoroaniline (27 mg, 0.19 mmol, 1.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 36% B to 37% B in 10 min, 37% B; Wave Length: 254/220 nm; RT1 (min): 10) to afford 3-[(3-chloro-2-fluorophenyl)amino]-2-[2-methoxypyrido[3,2-d]pyrimidin-8-yl]-1H,5H,6H,7H-pyrrolo[3,2-c]pyridin-4-one (14.8 mg, 17.45%) as a yellow solid.

LC-MS: (M+H)⁺ found: 438.95.

¹H NMR (400 MHz, DMSO-d6) δ 12.02 (s, 1H), 9.50 (s, 1H), 8.74 (d, J=8.0 Hz, 1H), 7.97 (s, 1H), 7.71 (d, J=4.0 Hz, 1H), 7.25 (s, 1H), 6.78-6.76 (m, 2H), 6.32-6.27 (m, 1H), 4.19 (s, 3H), 3.45 (t, J=8.0 Hz, 2H), 2.96 (t, J=8.0 Hz, 2H).

### Example 178. 3-[(3-chlorophenyl)amino]-2-[2-methoxypyrido[3,2-d]pyrimidin-8-yl]-1H,5H,6H,7H-pyrrolo[3,2-c]pyridin-4-one (compound 798)

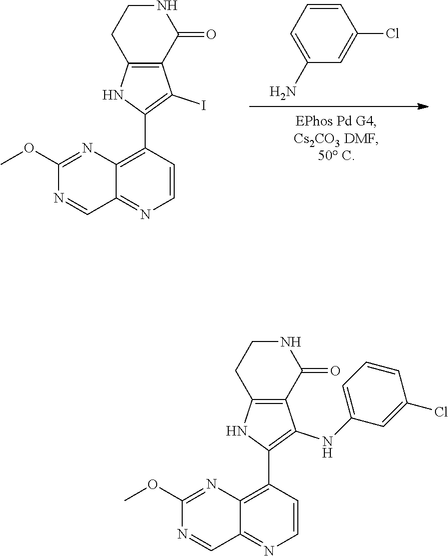 [SMILES: COc1ncc2nccc(-c3[nH]c4c(c3I)C(=O)NCC4)c2n1 ; Nc1cccc(Cl)c1 ; COc1ncc2nccc(-c3[nH]c4c(c3Nc3cccc(Cl)c3)C(=O)NCC4)c2n1]

To a stirred mixture of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) and 3-chloroaniline (24 mg, 0.19 mmol, 1.00 equiv) and Ephos Pd g4 (17 mg, 0.019 mmol, 0.1 equiv) and Cs2CO3 (123 mg, 0.38 mmol, 2 equiv) in DMF (1.90 mL) in portions at room temperature under argon atmosphere. The resulting mixture was stirred for 2 h at 50 degrees C. under argon atmosphere. Desired product could be detected by LCMS. The crude product (80 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 14% B to 44% B in 7 min; Wave Length: 254 nm; RT1 (min): 6.5) to afford 3-[(3-chlorophenyl)amino]-2-[2-methoxypyrido[3,2-d]pyrimidin-8-yl]-1H,5H,6H,7H-pyrrolo[3,2-c]pyridin-4-one (11.8 mg, 14.72%) as a yellow solid.

LC-MS: (M+H)⁺ found: 421.

¹H NMR (300 MHz, DMSO-d6) δ 12.05 (s, 1H), 9.49 (s, 1H), 8.71 (d, J=4.8 Hz, 1H), 7.99 (s, 1H), 7.72 (d, J=4.8 Hz, 1H), 7.21 (t, J=2.6 Hz, 1H), 7.02 (t, J=8.3 Hz, 1H), 6.68-6.58 (m, 2H), 6.56-6.51 (m, 1H), 4.20 (s, 3H), 3.46 (td, J=6.8, 2.5 Hz, 2H), 2.97 (t, J=6.8 Hz, 2H)

### Example 179. 3-[(2,6-difluorophenyl)amino]-2-[2-methoxypyrido[3,2-d]pyrimidin-8-yl]-1H,5H,6H,7H-pyrrolo[3,2-c]pyridin-4-one (compound 806)

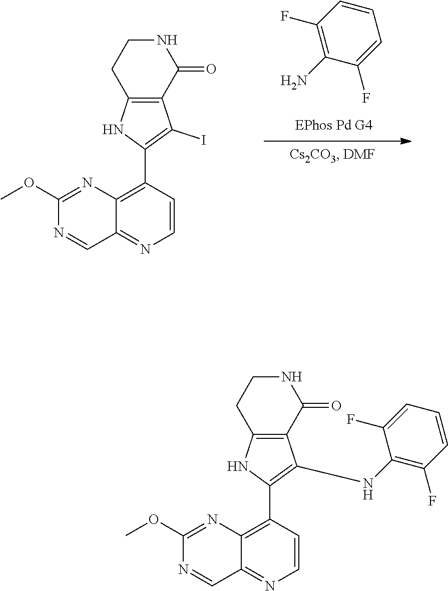 [SMILES: COc1ncc2nccc(-c3[nH]c4c(c3I)C(=O)NCC4)c2n1 ; Nc1c(F)cccc1F ; COc1ncc2nccc(-c3[nH]c4c(c3Nc3c(F)cccc3F)C(=O)NCC4)c2n1]

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 2,6-difluoroaniline (73 mg, 0.57 mmol, 3.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 14% B to 38% B in 10 min, 38% B; Wave Length: 254/220 nm; RT1 (min): 9.67) to afford 3-[(2,6-difluorophenyl)amino]-2-[2-methoxypyrido[3,2-d]pyrimidin-8-yl]-1H,5H,6H,7H-pyrrolo[3,2-c]pyridin-4-one (10.4 mg, 12.79%) as a yellow solid.

LC-MS: (M+H)⁺ found: 422.95.

¹H NMR (400 MHz, DMSO-d6) δ 11.63 (s, 1H), 9.43 (s, 1H), 8.63 (d, J=4.0 Hz, 1H), 7.68 (d, J=8.0 Hz, 1H), 7.54 (s, 1H), 7.06 (s, 1H), 6.80-6.75 (m, 2H), 6.69-6.67 (m, 1H), 4.14 (s, 3H), 3.44-3.32 (m, 2H), 2.91 (t, J=4.0 Hz, 2H).

### Example 180. 3-[(2,3-difluorophenyl)amino]-2-[2-methoxypyrido[3,2-d]pyrimidin-8-yl]-1H,5H,6H,7H-pyrrolo[3,2-c]pyridin-4-one (compound 805)

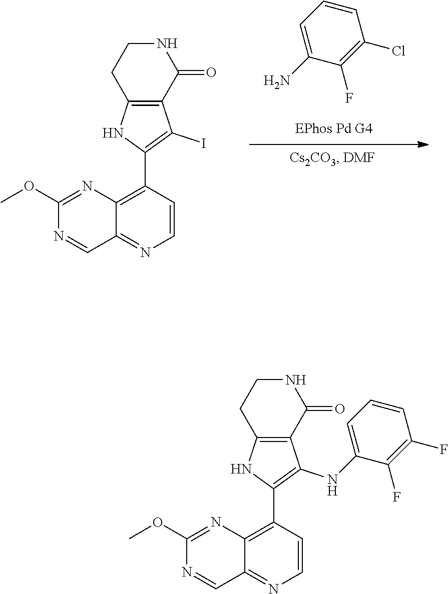 [SMILES: COc1ncc2nccc(-c3[nH]c4c(c3I)C(=O)NCC4)c2n1 ; Nc1cccc(Cl)c1F ; COc1ncc2nccc(-c3[nH]c4c(c3Nc3cccc(F)c3F)C(=O)NCC4)c2n1]

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 2,3-difluoroaniline (73 mg, 0.57 mmol, 3.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep C18 OBD Column, 30*100 mm, 5|Ìm; Mobile Phase A: Water (10 mmol/L NH4HCO3), Mobile Phase B: ACN; Flow rate: 60 mL/min; Gradient: 23% B to 47% B in 9 min, 47% B; Wave Length: 254/220 nm; RT1 (min): 8.85) to afford 3-[(2,3-difluorophenyl)amino]-2-[2-methoxypyrido[3,2-d]pyrimidin-8-yl]-1H,5H,6H,7H-pyrrolo[3,2-c]pyridin-4-one (11.8 mg, 14.52%) as a yellow solid.

LC-MS: (M+H)⁺ found: 423.

¹H NMR (400 MHz, DMSO-d6) δ 11.98 (s, 1H), 9.49 (s, 1H), 8.73 (d, J=8.0 Hz, 1H), 7.94 (s, 1H), 7.72 (d, J=8.0 Hz, 1H), 7.24 (s, 1H), 6.71-6.63 (m, 2H), 6.20-6.14 (m, 1H), 4.18 (s, 3H), 3.47-3.43 (m, 2H), 2.95 (t, J=8.0 Hz, 2H).

### Example 181. 3-[(2-fluoro-3-methylphenyl)amino]-2-[2-methoxypyrido[3,2-d]pyrimidin-8-yl]-1H,5H,6H,7H-pyrrolo[3,2-c]pyridin-4-one (compound 804)

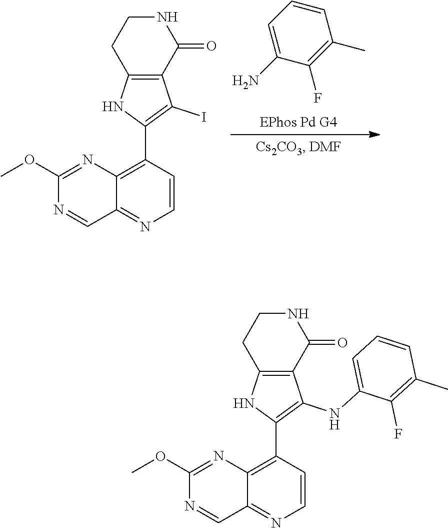

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 2-fluoro-3-methylaniline (71 mg, 0.57 mmol, 3.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep OBD C18 Column, 30*150 mm, 5|Ìm; Mobile Phase A: Water (10 mmol/L NH4HCO3), Mobile Phase B: ACN; Flow rate: 60 mL/min; Gradient: 28% B to 58% B in 7 min; Wave Length: 254 nm; RT1 (min): 6.5) to afford 3-[(2-fluoro-3-methylphenyl)amino]-2-[2-methoxypyrido[3,2-d]pyrimidin-8-yl]-1H,5H,6H,7H-pyrrolo[3,2-c]pyridin-4-one (10.9 mg, 13.55%) as a yellow solid.

LC-MS: (M+H)⁺ found: 419.

¹H NMR (400 MHz, DMSO-d6) δ 11.97 (s, 1H), 9.48 (s, 1H), 8.66 (d, J=4.0 Hz, 1H), 7.80 (d, J=4.0 Hz, 1H), 7.53 (d, J=4.0 Hz, 1H), 7.30 (s, 1H), 6.59-6.54 (m, 2H), 6.14-6.12 (m, 1H), 4.20 (s, 3H), 3.48-3.44 (m, 2H), 2.96 (t, J=4.0 Hz, 2H), 2.22 (d, J=4.0 Hz, 3H).

### Example 182. 3-[(2,3-dichlorophenyl)amino]-2-[2-methoxypyrido[3,2-d]pyrimidin-8-yl]-1H,5H,6H,7H-pyrrolo[3,2-c]pyridin-4-one (compound 803)

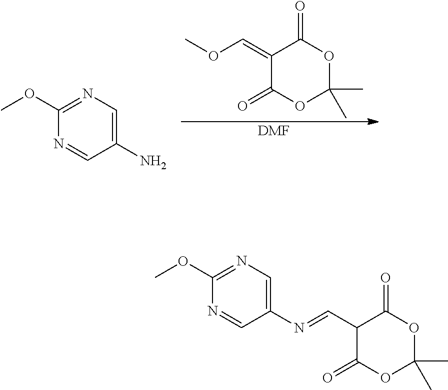

To a stirred solution of 2-methoxypyrimidin-5-amine (2.00 g, 15.983 mmol, 1.00 equiv) in DMF (16.00 mL) was added 5-(methoxymethylidene)-2,2-dimethyl-1,3-dioxane-4,6-dione (3.27 g, 17.582 mmol, 1.1 equiv) at room temperature under nitrogen atmosphere. The resulting mixture was stirred for 1 h at 80 degrees C. under nitrogen atmosphere. The reaction was monitored by LCMS. The resulting mixture was diluted with MeOH (50 ml). The precipitated solids were collected by filtration and washed with MeOH (3×50 mL). This resulted in 5-[(1E)-[(2-methoxypyrimidin-5-yl)imino]methyl]-2,2-dimethyl-1,3-dioxane-4,6-dione (3.9 g, 87.38%) as a light yellow solid.

LC-MS: M+H found: 280.

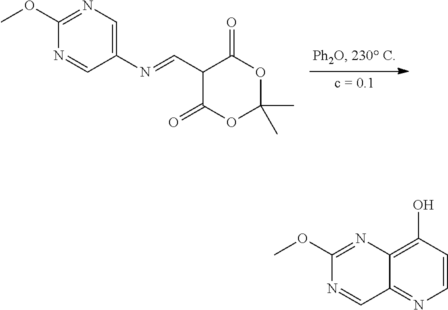

A solution of 5-[(1E)-[(2-methoxypyrimidin-5-yl)imino]methyl]-2,2-dimethyl-1,3-dioxane-4,6-dione (2.00 g, 7.162 mmol, 1.00 equiv) in diphenyl-ether (70.00 mL) was stirred for 1 h at 230 degrees C. under nitrogen atmosphere. The reaction was monitored by LCMS. The resulting mixture was diluted with hexane (50 mL). The precipitated solids were collected by filtration and washed with hexane (3×20 mL). The crude product (1.26 g) was used in the next step directly without further purification.

LC-MS: M+H found: 178.

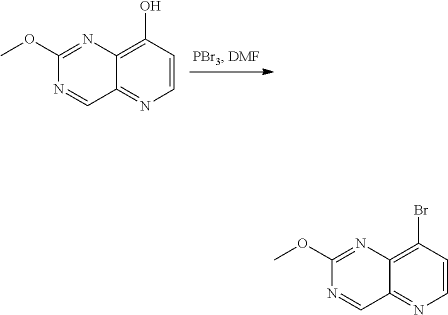

To a stirred solution of 2-methoxypyrido[3,2-d]pyrimidin-8-ol (1.26 g, 7.112 mmol, 1.00 equiv) in DMF (18.00 mL) was added phosphorus tribromide (2.31 g, 8.535 mmol, 1.20 equiv) dropwise at 0 degrees C. under nitrogen atmosphere. The resulting mixture was stirred for 1 h at room temperature under nitrogen atmosphere. The reaction was monitored by LCMS. LCMS showed the reaction was completed. The mixture was basified to pH 7 with saturated Na2CO3 (aq.). The resulting mixture was extracted with EtOAc (3×150 mL). The combined organic layers were washed with brine (2×250 mL), dried over anhydrous Na2SO4. After filtration, the filtrate was concentrated under reduced pressure. The residue was purified by Prep-TLC (PE/EtOAc 1.1) to afford 8-bromo-2-methoxypyrido[3,2-d]pyrimidine (40 mg, 2.34%) as a off-white solid.

LC-MS: M+H found: 240.

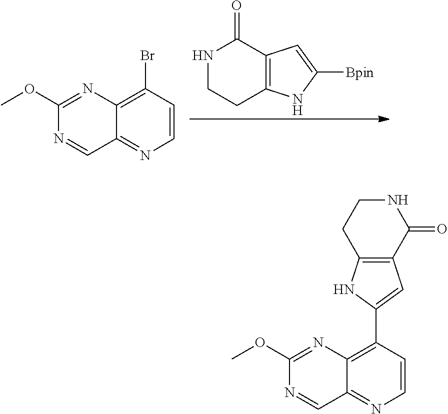

To a stirred solution of 8-bromo-2-methoxypyrido[3,2-d]pyrimidine (110 mg, 0.45 mmol, 1.00 equiv) in dioxane (3.50 mL) was added 2-(4,4,5,5-tetramethyl-1,3,2-dioxaborolan-2-yl)-1H,5H,6H,7H-pyrrolo[3,2-c]pyridin-4-one (240 mg, 0.91 mmol, 2.00 equiv) and Na₂CO₃ (145 mg, 1.37 mmol, 3.00 equiv) and Pd(PPh₃)₄ (52 mg, 0.046 mmol, 0.10 equiv) in portions at room temperature under nitrogen atmosphere. To the above mixture was added H2O (0.70 mL) dropwise at room temperature. The resulting mixture was stirred for additional overnight at 50 degrees C. The reaction was monitored by LCMS. LCMS showed the reaction was completed. The resulting mixture was concentrated under reduced pressure. The residue was purified by silica gel column chromatography, eluted with CH2Cl2/MeOH (10:1) to afford 2-[2-methoxypyrido[3,2-d]pyrimidin-8-yl]-1H,5H,6H,7H-pyrrolo[3,2-c]pyridin-4-one (120 mg, 88.68%) as a yellow solid.

LC-MS: M+H found: 296.

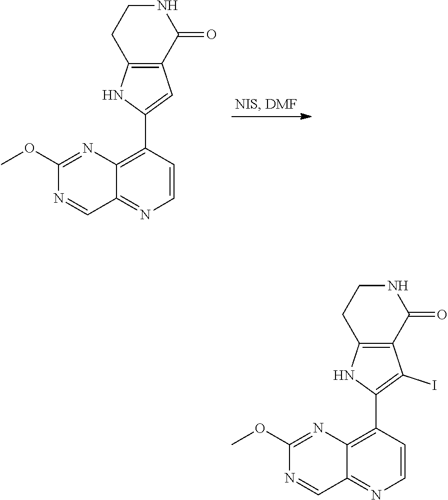 [SMILES: COc1ncc2nccc(-c3cc4c([nH]3)CCNC4=O)c2n1 ; COc1ncc2nccc(-c3[nH]c4c(c3I)C(=O)NCC4)c2n1]

To a stirred solution of 2-[2-methoxypyrido[3,2-d]pyrimidin-8-yl]-1H,5H,6H,7H-pyrrolo[3,2-c]pyridin-4-one (120 mg, 0.40 mmol, 1.00 equiv) in DMF (6.00 mL) was added N-iodosuccinimide (109 mg, 0.48 mmol, 1.20 equiv) in portions at room temperature under nitrogen atmosphere. The resulting mixture was stirred for 24 h at room temperature under nitrogen atmosphere. The reaction was monitored by LCMS. LCMS showed the reaction was completed. The reaction was quenched by the addition of sat. Na2SO3 (aq.) (5 mL) at room temperature. The mixture was stirred for 10 min at room temperature under nitrogen atmosphere. The mixture was basified to pH 7 with saturated Na2CO3 (aq.). The resulting mixture was extracted with CH2Cl2: MeOH (10:1) 3×50 mL). The combined organic layers were washed with brine (2×20 mL), dried over anhydrous Na2SO4. After filtration, the filtrate was concentrated under reduced pressure. The resulting mixture was concentrated under reduced pressure. The residue was purified by reverse flash chromatography with the following conditions: column, C18 silica gel; mobile phase, ACN in water, 0% to 100% gradient in 40 min; detector, UV 254 nm. The resulting mixture was concentrated under vacuum to afford 3-iodo-2-[2-methoxypyrido[3,2-d]pyrimidin-8-yl]-1H,5H,6H,7H-pyrrolo[3,2-c]pyridin-4-one (100 mg, 58.42%) as an orange solid.

LC-MS: M+H found: 422.

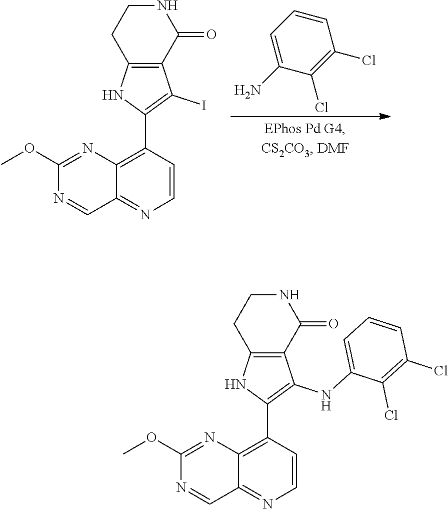 [SMILES: COc1ncc2nccc(-c3[nH]c4c(c3I)C(=O)NCC4)c2n1 ; Nc1cccc(Cl)c1Cl ; COc1ncc2nccc(-c3[nH]c4c(c3Nc3cccc(Cl)c3Cl)C(=O)NCC4)c2n1]

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) and 2,3-dichloroaniline (30 mg, 0.19 mmol, 1.00 equiv) in DMF (2.00 mL) were added EPhos Pd G4 (17 mg, 0.019 mmol, 0.10 equiv) and Cs₂CO₃ (123 mg, 0.38 mmol, 2.00 equiv) under Ar atmosphere. The resulting mixture was stirred for 2 hrs at 50 degrees C. under Ar atmosphere. The reaction was monitored by LCMS. LCMS showed the reaction was completed. The resulting mixture was concentrated under vacuum. The residue was purified by silica gel column chromatography, eluted with CH₂Cl₂/MeOH (10:1) to afford crude product. The crude product was purified by Prep-HPLC with the following conditions (Column: XBridge Prep C18 OBD Column, 30*100 mm, 5 um: Mobile Phase A: Water (10 mmol/L NH₄HCO₃), Mobile Phase B: ACN; Flow rate: 60 mL/min; Gradient: 26% B to 50% B in 9 min, 50% B; Wave Length: 254/220 nm; RT1 (min): 9.67) to afford 3-[(2,3-dichlorophenyl)amino]-2-[2-methoxypyrido[3,2-d]pyrimidin-8-yl]-1H,5H,6H,7H-pyrrolo[3,2-c]pyridin-4-one (8.3 mg, 9.30%) as an orange solid.

LC-MS: M+H found: 454.95.

¹H NMR (400 MHz, DMSO-d6) δ 12.03 (s, 1H), 9.50 (s, 1H), 8.73 (d, J=4.8 Hz, 1H), 8.04 (s, 1H), 7.59 (d, J=4.8 Hz, 1H), 7.25 (s, 1H), 6.93-6.83 (m, 2H), 6.32 (t, J=4.8 Hz, 1H), 4.20 (s, 3H), 3.52-3.43 (m, 2H), 2.97 (t, J=6.8 Hz, 2H).

### Example 183. 3-[[3-chloro-2-(trifluoromethoxy)phenyl]amino]-2-[2-methoxypyrido[3,2-d]pyrimidin-8-yl]-1H,5H,6H,7H-pyrrolo[3,2-c]pyridin-4-one (compound 802)

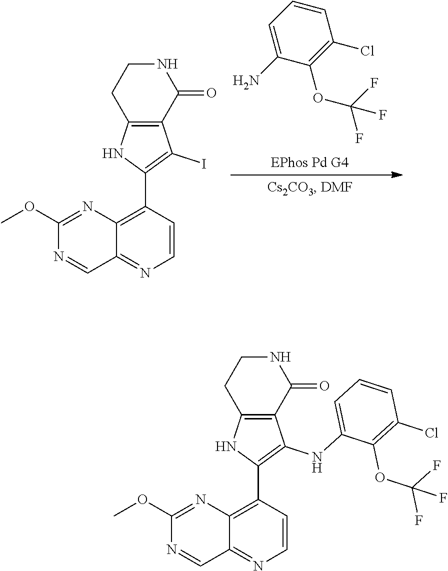

To a stirred solution of 3-iodo-2-[2-methoxypyrido[3,2-d]pyrimidin-8-yl]-1H,5H,6H,7H-pyrrolo[3,2-c]pyridin-4-one (80 mg, 0.19 mmol, 1.00 equiv) in DMF (2.00 mL) was added Cs₂CO₃ (123 mg, 0.38 mmol, 2.00 equiv) and EPhos Pd G4 (17 mg, 0.01 mmol, 0.10 equiv) in portions at room temperature under nitrogen atmosphere. To the above mixture was added 3-chloro-2-(trifluoromethoxy) aniline (40 mg, 0.19 mmol, 1.00 equiv) dropwise at room temperature. The resulting mixture was stirred for additional 2 h at 50 degrees C. The reaction was monitored by LCMS. LCMS showed the reaction was completed. The resulting mixture was concentrated under vacuum. The residue was purified by silica gel column chromatography, eluted with CH₂Cl₂/MeOH (10:1) to afford crude product. The crude product was purified by Prep-HPLC with the following conditions (Column: XBridge Prep C18 OBD Column, 30*100 mm, 5|Ìm; Mobile Phase A: Water (10 mmol/L NH4HCO3), Mobile Phase B: ACN; Flow rate: 60 mL/min; Gradient: 32% B to 55% B in 9 min, 55% B; Wave Length: 254/220 nm; RT1 (min): 8.85) to afford 3-[[3-chloro-2-(trifluoromethoxy)phenyl]amino]-2-[2-methoxypyrido[3,2-d]pyrimidin-8-yl]-1H,5H,6H,7H-pyrrolo[3,2-c]pyridin-4-one (2.4 mg, 2.48%) as a yellow solid.

LC-MS: (M+H)⁺ found: 504.95.

¹H NMR (400 MHz, Methanol-d4) δ 9.40 (s, 1H), 8.64-8.50 (m, 1H), 7.68 (s, 1H), 6.84 (d, J=4.0 Hz, 2H), 6.52-6.40 (m, 1H), 4.27 (s, 3H), 3.62-3.59 (m, 3H), 3.12-3.06 (m, 2H), 1.18 (t, J=8.0 Hz, 2H).

### Example 184. 3-{[3-chloro-2-(trifluoromethyl)phenyl]amino}-2-{2-methoxypyrido[3,2-d]pyrimidin-8-yl}-1H,5H,6H,7H-pyrrolo[3,2-c]pyridin-4-one (compound 801)

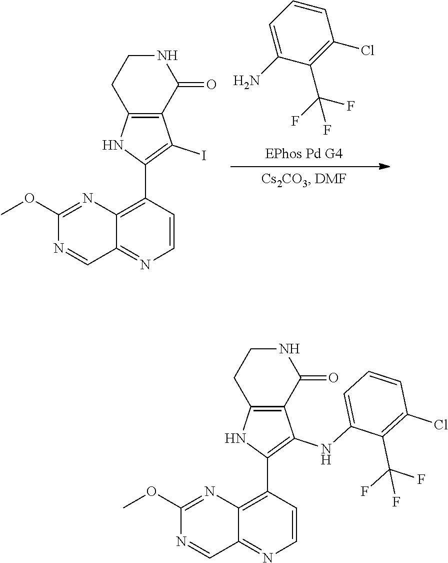

To a stirred solution of 3-iodo-2-{2-methoxypyrido[3,2-d]pyrimidin-8-yl}-1H,5H,6H,7H-pyrrolo[3,2-c]pyridin-4-one (60 mg, 0.14 mmol, 1.00 equiv) in 2 ml DMF was added Cs₂CO₃ (92 mg, 0.28 mmol, 2.00 equiv) and EPhos Pd G4 (13 mg, 0.01 mmol, 0.10 equiv) in portions at room temperature under nitrogen atmosphere. To the above mixture was added 3-chloro-2-(trifluoromethyl) aniline (27 mg, 0.14 mmol, 1.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by silica gel column chromatography, eluted with CH₂Cl₂/MeOH (10:1) to afford crude product. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 35% B to 52% B in 8 min, 52% B; Wave Length: 220/254 nm; RT1 (min): 7.97) to afford 3-{[3-chloro-2-(trifluoromethyl)phenyl]amino)-2-(2-methoxypyrido[3,2-d]pyrimidin-8-yl}-1H,5H,6H,7H-pyrrolo[3,2-c]pyridin-4-one (2.3 mg, 3.27%) as a yellow solid.

LC-MS: (M+H)⁺ found: 488.9.

¹H NMR (300 MHz, DMSO-d6) δ 12.02 (s, 1H), 9.50 (s, 1H), 8.72 (d, J=8.0 Hz, 1H), 8.08 (s, 1H), 7.60 (d, J=8.0 Hz, 1H), 7.23 (s, 1H), 7.07 (t, J=8.0 Hz, 1H), 6.88 (d, J=8.0 Hz, 1H), 6.55 (d, J=8.0 Hz, 1H), 4.18 (s, 3H), 3.47 (d, J=8.0 Hz, 2H), 2.97 (t, J=8.0 Hz, 2H).

### Example 185. 2-(6,7-dimethoxy-1,5-naphthyridin-4-yl)-3-[(3-fluoro-2-methylphenyl)amino]-1H,5H,6H,7H-pyrrolo[3,2-c]pyridin-4-one (compound 808)

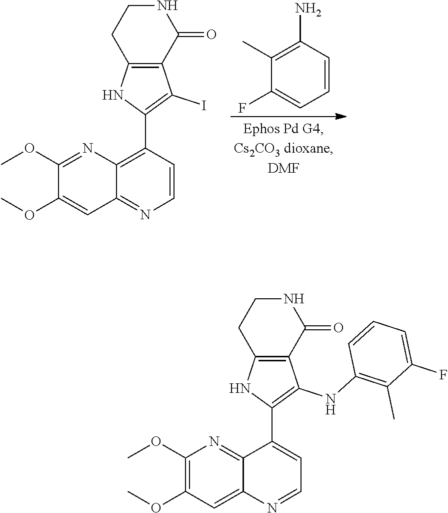

To a stirred mixture of 2-(6,7-dimethoxy-1,5-naphthyridin-4-yl)-3-iodo-1H,5H,6H,7H-pyrrolo[3,2-c]pyridin-4-one (90 mg, 0.20 mmol, 1.00 equiv) and 3-fluoro-2-methyl-aniline (125 mg, 1.0 mmol, 5 equiv) in DMF (2.00 mL) were added Ephos Pd G4 (18 mg, 0.02 mmol, 0.1 equiv) and Cs₂CO₃ (130 mg, 0.40 mmol, 2 equiv) in portions at 50 degrees C. under argon atmosphere. The resulting mixture was stirred for overnight at 50 degrees C. under argon atmosphere. The resulting mixture was concentrated under reduced pressure. The residue was purified by Prep-TLC (CH₂Cl₂/MeOH 10:1) to afford crude product as a light yellow solid. The crude product (50 mg) was purified by Prep-HPLC with the following conditions (Column: XBridge Prep C18 OBD Column, 30*100 mm, 5|Ìm; Mobile Phase A: Water (10 mmol/L NH4HCO3), Mobile Phase B: ACN; Flow rate: 60 mL/min; Gradient: 26% B to 50% B in 9 min, 50% B; Wave Length: 254/220 nm; RT1 (min): 8.98) to afford 2-(6,7-dimethoxy-1,5-naphthyridin-4-yl)-3-[(3-fluoro-2-methylphenyl)amino]-1H,5H,6H,7H-pyrrolo[3,2-c]pyridin-4-one (23.3 mg, 25.50%) as a light yellow solid.

LC-MS: (M+H)⁺ found: 448.25.

¹H NMR (300 MHz, DMSO-d6) δ 12.04 (s, 1H), 8.44 (d, J=5.0 Hz, 1H), 7.65 (s, 1H), 7.50 (s, 1H), 7.34 (d, J=5.0 Hz, 1H), 7.23 (d, J=2.5 Hz, 1H), 6.74 (q, J=7.9 Hz, 1H), 6.48 (t, J=8.8 Hz, 1H), 6.10 (d, J=8.2 Hz, 1H), 4.21 (s, 3H), 3.99 (s, 3H), 3.46 (d, J=2.5 Hz, 2H), 2.95 (t, J=6.8 Hz, 2H), 2.23 (d, J=1.7 Hz, 3H).

### Example 186. 2-(6,7-dimethoxy-1,5-naphthyridin-4-yl)-3-[(3-fluoro-2-methoxyphenyl)amino]-1H,5H,6H,7H-pyrrolo[3,2-c]pyridin-4-one (compound 807)

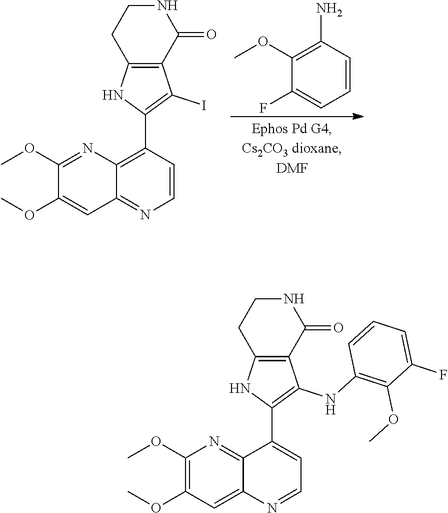

To a stirred mixture of 2-(6,7-dimethoxy-1,5-naphthyridin-4-yl)-3-iodo-1H,5H,6H,7H-pyrrolo[3,2-c]pyridin-4-one (100 mg, 0.22 mmol, 1.00 equiv) and 3-fluoro-2-methoxyaniline (157 mg, 1.1 mmol, 5.00 equiv) in DMF (2.00 mL) were added Ephos Pd G4 (20 mg, 0.02 mmol, 0.10 equiv) and Cs₂CO₃ (145 mg, 0.44 mmol, 2.00 equiv) in portions at 50 degrees C. under argon atmosphere. The resulting mixture was stirred for overnight at 50 degrees C. under argon atmosphere. The resulting mixture was concentrated under reduced pressure. The residue was purified by Prep-TLC (CH₂Cl₂/MeOH 10:1) to afford crude product as a light yellow solid. The crude product (50 mg) was purified by Prep-HPLC with the following conditions (Column: XBridge Prep C18 OBD Column, 30*100 mm, 5|Ìm; Mobile Phase A: Water (10 mmol/L NH4HCO3), Mobile Phase B: ACN; Flow rate: 60 mL/min; Gradient: 23% B to 45% B in 10 min, 45% B; Wave Length: 254/220 nm; RT1 (min): 9.67) to afford 2-(6,7-dimethoxy-1,5-naphthyridin-4-yl)-3-[(3-fluoro-2-methoxyphenyl)amino]-1H,5H,6H,7H-pyrrolo[3,2-c]pyridin-4-one (19.4 mg, 18.68%) as a light yellow solid.

LC-MS: (M+H)⁺ found 464.25.

¹H NMR (300 MHz, DMSO-d6) δ 11.98 (s, 1H), 8.51 (d, J=4.9 Hz, 1H), 7.76 (s, 1H), 7.67 (s, 1H), 7.41 (d, J=4.9 Hz, 1H), 7.18 (d, J=2.5 Hz, 1H), 6.61 (m, J=6.1 Hz, 1H), 6.48 (m, J=1.5 Hz, 1H), 6.05 (m, J=1.4 Hz, 1H), 4.20 (s, 3H), 4.00 (s, 3H), 3.86 (d, J=1.0 Hz, 3H), 3.46 (m, J=4.0 Hz, 2H), 2.94 (t, J=6.8 Hz, 2H).

### Example 187. 3-[(3-chloro-2-methoxyphenyl)amino]-2-(1,5-naphthyridin-4-yl)-1H,5H,6H,7H-pyrrolo[3,2-c]pyridin-4-one (compound 683)

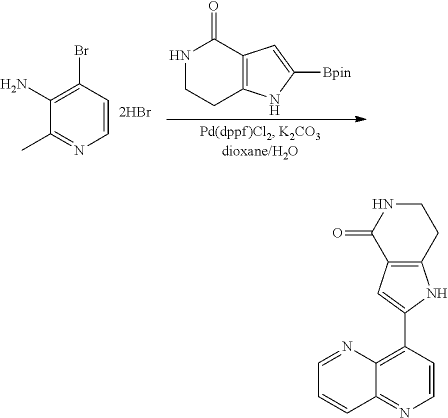

To a stirred solution of 4-bromo-1,5-naphthyridine (500 mg, 1.22 mmol, 1.00 equiv) and K₂CO₃ (956 mg, 6.80 mmol, 5.00 equiv) in 1,4-dioxane (5.00 mL) was added 2-(dihydroxymethyl)-1,5,6,7-tetrahydro-4H-pyrrolo[3,2-c] pyridin-4-one (426 mg, 1.58 mmol, 1.20 equiv) in portions at room temperature under Ar atmosphere. The resulting mixture was stirred for 4 h at 80 degrees C. under Ar atmosphere. The reaction was monitored by TLC and LCMS. The mixture was allowed to cool down to room temperature. The residue was purified by Prep-TLC (DCM/MeOH=10/1) to afford 2-(1,5-naphthyridin-4-yl)-1H,5H,6H,7H-pyrrolo[3,2-c] pyridin-4-one (250 mg, 69.0%) as a yellow solid.

LC-MS: M+H found: 265.05.

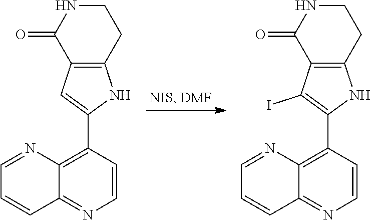 [SMILES: O=C1NCCc2[nH]c(-c3ccnc4cccnc34)cc21 ; O=C1NCCc2[nH]c(-c3ccnc4cccnc34)c(I)c21]

To a stirred solution of 2-(1,5-naphthyridin-4-yl)-1H,5H,6H,7H-pyrrolo[3,2-c] pyridin-4-one (200 mg, 0.75 mmol, 1.00 equiv) in DMF (6.00 mL) was added NIS (170 mg, 0.75 mmol, 1.00 equiv) in portions at 0 degrees C. under N2 atmosphere. The resulting mixture was stirred for 2 h at room temperature. The precipitated solids were collected by filtration and washed with CH₂Cl₂ (2×8 mL). The crude product (450 mg) was purified by silica gel column chromatography with the following conditions (DCM/MeOH=10/1) to afford 3-iodo-2-(1,5-naphthyridin-4-yl)-1H,5H,6H,7H-pyrrolo[3,2-c] pyridin-4-one (180 mg, 61.0%) as a white solid.

LC-MS: M+H found: 390.90.

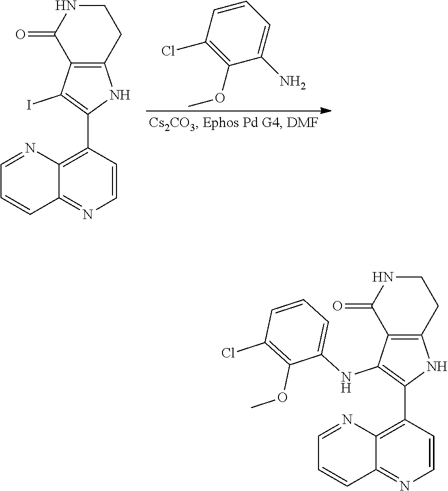

To a stirred solution of 3-iodo-2-(1,5-naphthyridin-4-yl)-1H,5H,6H,7H-pyrrolo[3,2-c]pyridin-4-one (100 mg, 0.03 mmol, 1.00 equiv) and 3-chloro-2-methoxyaniline (40 mg, 0.03 mmol, 1.00 equiv) in DMF (3.00 mL) was added Cs₂CO₃ (45 mg, 3.00 equiv) and Ephos Pd G4 (9 mg, 0.15 equiv) under Ar atmosphere. The resulting mixture was stirred for 2 h at 80 degrees C. The precipitated solids were collected by filtration and washed with MeOH. The crude product (80 mg) was purified by Prep-HPLC with the following conditions ((2 #SHIMADZU (HPLC-01): Column, XBridge Prep OBD C18 Column, 30*150 mm, 5 μm; mobile phase, Water (10 mmol/L NH₄HCO₃) and ACN (30% ACN up to 60% in 7 min), Mobile Phase B: ACN; Flow rate: 60 mL/min; Gradient: 40 B to 55 B in 8 min; 220 nm; RT: 6) to afford 3-[(3-chloro-2-methoxyphenyl)amino]-2-(1,5-naphthyridin-4-yl)-1H,5H,6H,7H-pyrrolo[3,2-c]pyridin-4-one (7.0 mg, 6.5%) as a yellow solid.

LC-MS: (M+H)⁺ found: 420.20.

¹H NMR (400 MHz, DMSO-d₆) δ 12.61 (s, 1H), 9.11 (dd, J=4.2, 1.8 Hz, 1H), 8.76 (d, J=4.9 Hz, 1H), 8.46 (dd, J=8.6, 1.8 Hz, 1H), 8.11 (s, 1H), 7.89 (dd, J=8.5, 4.2 Hz, 1H), 7.56 (d, J=4.7 Hz, 1H), 7.25 (s, 1H), 6.77-6.67 (m, 2H), 6.22 (dd, J=7.8, 1.9 Hz, 1H), 3.93 (s, 3H), 3.46 (td, J=6.8, 2.8 Hz, 2H), 3.01 (t, J=6.7 Hz, 2H).

### Example 188. 2-(7-fluoro-1,5-naphthyridin-4-yl)-3-[(3-fluoro-2-methoxyphenyl)amino]-1H,5H,6H,7H-pyrrolo[3,2-c]pyridin-4-one (compound 825)

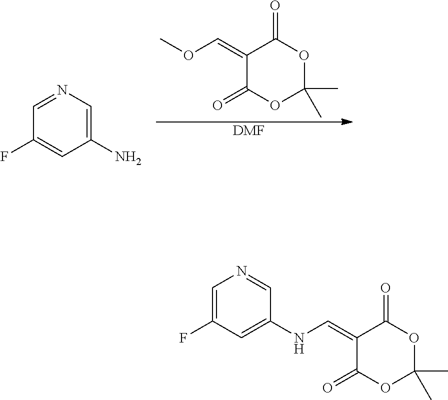

Into a dimethylformamide (80 mL) were added 5-fluoropyridin-3-amine (10 g, 89.2 mmol, 1.00 equiv) and 5-(methoxymethylidene)-2,2-dimethyl-1,3-dioxane-4,6-dione (19.9 g, 107.04 mmol, 1.20 equiv) at room temperature. The resulting mixture was stirred for 1 h at 80 degrees C. under N2 atmosphere. The reaction was monitored by TLC. The resulting mixture was washed with MeOH (16000 mL). The resulting mixture was filtered, the filter cake was washed with MeOH (10.00 mL). The filter cake was concentrated under reduced pressure to give 5-{[(5-fluoropyridin-3-yl)amino]methylidene}-2,2-dimethyl-1,3-dioxane-4,6-dione (23 g, 87.17%) as a white solid.

LC-MS: (M+H)⁺ found: 266.90.

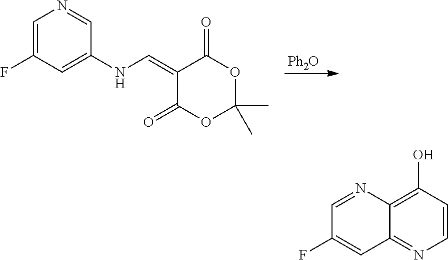

Into a diphenyl-ether (200 mL) were added 5-{[(5-fluoropyridin-3-yl)amino]methylidene}-2,2-dimethyl-1,3-dioxane-4,6-dione (5 g, 18.78 mmol, 1.00 equiv) at 230 degrees C. The resulting mixture was stirred for 10 min at 230 degrees C. The reaction was monitored by LCMS. The mixture was allowed to cool down to 50 degrees C. The resulting mixture was diluted with n-hexane (400 mL). The precipitated solids were collected by filtration and washed with n-hexane (10 mL) to give 7-fluoro-1,5-naphthyridin-4-ol (3 g, 87.59%) as a white solid.

LC-MS: (M+H)⁺ found: 165.

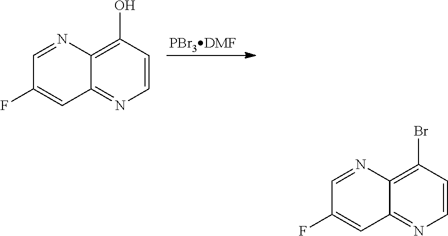 [SMILES: Oc1ccnc2cc(F)cnc12 ; Fc1cnc2c(Br)ccnc2c1]

Into a DMF (36.00 mL) were added 7-fluoro-1,5-naphthyridin-4-ol (3.6 g, 21.93 mmol, 1.00 equiv) and PBr₃ (6.53 g, 24.13 mmol, 1.10 equiv) at 0 degrees C. The resulting mixture was stirred for 1 h at room temperature under N2 atmosphere. The reaction was monitored by LCMS. Desired product could be detected by LCMS. The reaction was quenched with NaHCO₃solution (50.00 mL) at 0 degrees C. The resulting mixture was extracted with EA (3×100.00 mL). The combined organic layers were washed with NaCl solution (3×150 mL), dried over anhydrous Na₂SO₄. After filtration, the filtrate was concentrated under reduced pressure. The residue was purified by silica gel column chromatography, eluted with EA/PE (0 to 100%) to afford 8-bromo-3-fluoro-1,5-naphthyridine (3.5 g, 63.26%) as a white solid.

LC-MS: (M+H)⁺ found: 165.

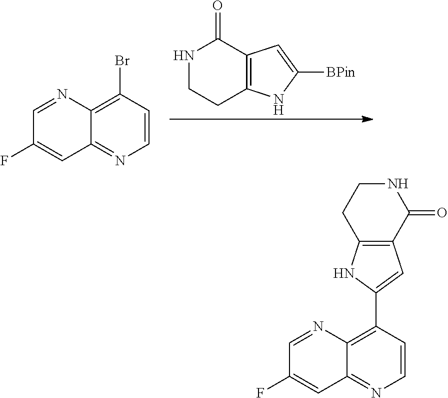

To a stirred solution of 8-bromo-3-fluoro-1,5-naphthyridine (1 g, 4.40 mmol, 1.00 equiv) and 2-(4,4,5,5-tetramethyl-1,3,2-dioxaborolan-2-yl)-1H,5H,6H,7H-pyrrolo[3,2-c]pyridin-4-one (1.73 g, 6.60 mmol, 1.5 equiv) in dioxane (20.00 mL) and H₂O (4.00 mL) were added Pd(PPh₃)₄ (0.51 g, 0.44 mmol, 0.10 equiv) and Na2CO3 (1.40 g, 13.21 mmol, 3.00 equiv) dropwise at RT under N2 atmosphere. The resulting mixture was stirred for 1 h at 50 degrees C. under N2 atmosphere. The reaction was monitored by LCMS. The resulting mixture was concentrated under reduced pressure. The residue was purified by silica gel column chromatography, eluted with MeOH/DCM (0 to 10%) to afford 2-(7-fluoro-1,5-naphthyridin-4-yl)-1H,5H,6H,7H-pyrrolo[3,2-c]pyridin-4-one (300 mg, 22.92%) as a as a yellow solid.

LC-MS: (M+H)⁺ found: 283.

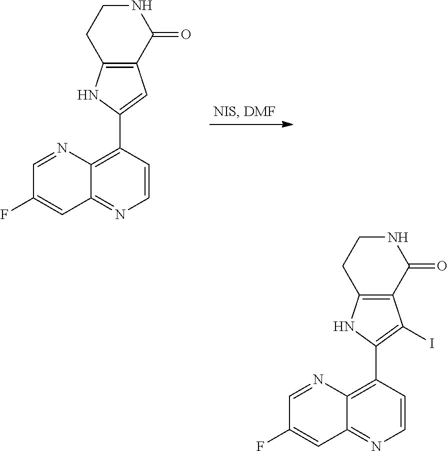 [SMILES: O=C1NCCc2[nH]c(-c3ccnc4cc(F)cnc34)cc21 ; O=C1NCCc2[nH]c(-c3ccnc4cc(F)cnc34)c(I)c21]

Into a DMF (3.00 mL) were 2-(7-fluoro-1,5-naphthyridin-4-yl)-1H,5H,6H,7H-pyrrolo[3,2-c]pyridin-4-one (150 mg, 0.53 mmol, 1.00 equiv) and NIS (119 mg, 0.53 mmol, 1.00 equiv) at room temperature. The resulting mixture was stirred for 1 h at room temperature. The reaction was monitored by LCMS. The resulting mixture was concentrated under reduced pressure. The residue was purified by silica gel column chromatography, eluted with MeOH/DCM (3%) to afford 2-(7-fluoro-1,5-naphthyridin-4-yl)-3-iodo-1H,5H,6H,7H-pyrrolo[3,2-c]pyridin-4-one (160 mg, 70.08%) as a yellow solid.

LC-MS: MH (C15H10FIN4O)=408; should be 408.

LC-MS: (M+H)⁺ found: 283.

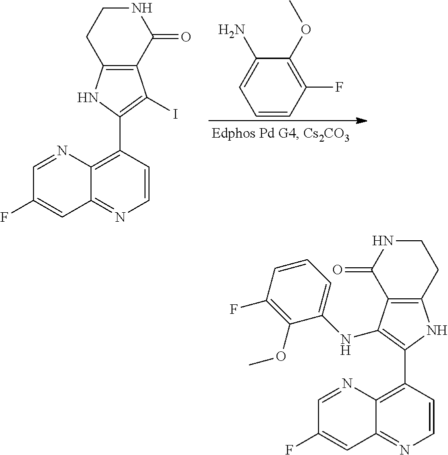

To a stirred solution of 2-(7-fluoro-1,5-naphthyridin-4-yl)-3-iodo-1H,5H,6H,7H-pyrrolo[3,2-c]pyridin-4-one (80 mg, 0.19 mmol, 1.00 equiv) and 3-fluoro-2-methoxyaniline (83 mg, 0.59 mmol, 3.00 equiv) in DMF (1.00 mL) was added Ephos Pd G4 (18 mg, 0.02 mmol, 0.10 equiv) dropwise at RT under Ar atmosphere. The resulting mixture was stirred for 1 h at 50 degrees C. under Ar atmosphere. The reaction was monitored by LCMS. The crude product was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 30% B to 46% B in 8 min, 46% B; Wave Length: 254/220 nm; RT1 (min): 7.8) to afford 2-(7-fluoro-1,5-naphthyridin-4-yl)-3-[(3-fluoro-2-methoxyphenyl)amino]-1H,5H,6H,7H-pyrrolo[3,2-c]pyridin-4-one (43.7 mg, 52.22%) as a yellow solid.

LC-MS: (M+H)⁺ found: 422.20.

¹H NMR (400 MHz, DMSO-d6) δ 12.36 (s, 1H), 9.12 (d, J=2.4 Hz, 1H), 8.76 (d, J=4.0 Hz, 1H), 8.33-8.31 (m, 1H), 8.04 (s, 1H), 7.56 (d, J=4.8 Hz, 1H), 7.25 (s, 1H), 6.66-6.10 (m, 1H), 6.56 (t, J=8.8 Hz, 1H), 6.05 (d, J=8.0 Hz, 1H), 3.94 (s, 3H), 3.50-3.42 (m, 2H), 2.98 (t, J=6.4 Hz, 2H).

### Example 189. 3-[(3-chloro-2-methoxyphenyl)amino]-2-{2-ethoxypyrido[3,2-d]pyrimidin-8-yl}-1H,5H,6H,7H-pyrrolo[3,2-c]pyridin-4-one (compound 827)

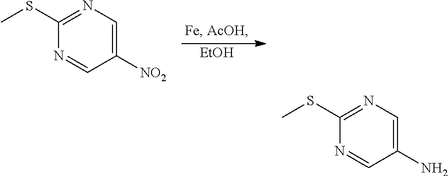

To a stirred solution of 2-(methylsulfanyl)-5-nitropyrimidine (5 g, 29.211 mmol, 1.00 equiv) in EtOH (200 mL) was added AcOH (120 mL) and Fe (17 g, 292.11 mmol, 10 equiv) in portions at rt under N2 atmosphere. The resulting mixture was stirred for 2 h at 80 degrees C. under N₂ atmosphere. Desired product could be detected by LCMS. LC-MS: M+H found: 142. The resulting mixture was concentrated under reduced pressure. The resulting mixture was extracted with EA (3×100 mL). The combined organic layers were washed with Saturated NaCl (3×100 mL), dried over anhydrous Na₂SO₄. After filtration, the filtrate was concentrated under reduced pressure to afford 2-(methylsulfanyl)pyrimidin-5-amine (3.5 g, 84.86%) as a yellow solid.

LC-MS: M+H found: 142.0.

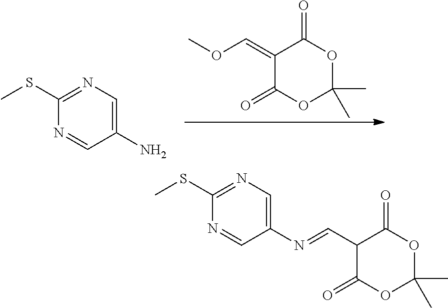

To a stirred solution of 2-(methylsulfanyl)pyrimidin-5-amine (3.2 g, 22.66 mmol, 1.00 equiv) and 5-(methoxymethylidene)-2,2-dimethyl-1,3-dioxane-4,6-dione (5.06 g, 27.18 mmol, 1.20 equiv) in DMF (80.00 mL) at rt under N2 atmosphere. The resulting mixture was stirred for 1 h at 80 degrees C. under N2atmosphere. Desired product could be detected by LCMS. LC-MS: M+H found: 296. The resulting mixture was added MeOH (50 mL). The resulting mixture was filtered, the filter cake was washed with MeOH (3×20 mL). The filter cake was concentrated under reduced pressure to afford 2,2-dimethyl-5-[(1E)-[[2-(methylsulfanyl)pyrimidin-5-yl]imino]methyl]-1,3-dioxane-4,6-dione (5.4 g, 80.68%) as a yellow solid.

LC-MS: (M+H)⁺ found 296.0.

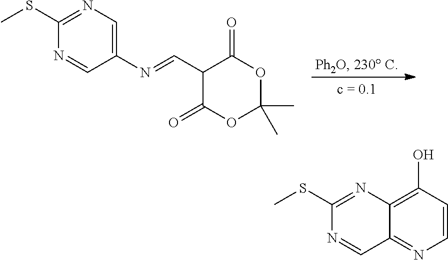

To a stirred solution of 2,2-dimethyl-5-[(1E)-{[2-(methylsulfanyl)pyrimidin-5-yl]imino}methyl]-1,3-dioxane-4,6-dione (5.3 g, 17.95 mmol, 1.00 equiv) in phenoxybenzene (360 mL) at rt under N2 atmosphere. The resulting mixture was stirred at 230 degrees C. under N2 atmosphere. Desired product could be detected by LCMS. LC-MS: M+H found: 194. The reaction was addition of Hexane (700 ml) at rt. The resulting mixture was filtered; the filter cake was washed with Hexane (3×200 ml). The filtrate was concentrated under reduced pressure.

LC-MS: (M+H)⁺ found: 194.0.

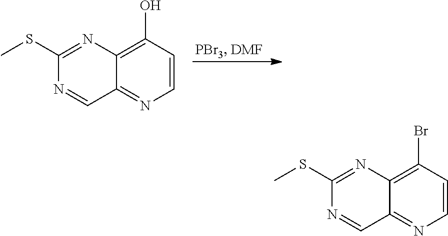

To a stirred solution of 2-(methylsulfanyl)pyrido[3,2-d]pyrimidin-8-ol (2.8 g, 14.49 mmol, 1.00 equiv) in DMF (80 mL) was added PBr₃ (4.31 g, 15.94 mmol, 1.1 equiv) dropwise at 0 degrees C. under N2 atmosphere. The resulting mixture was stirred for 1 h at rt under N2 atmosphere. Desired product could be detected by LCMS. LC-MS: M+H found: 256. The resulting mixture was extracted with EA (3×100 mL). The combined organic layers were washed with aq. NaCl (3×100 mL), dried over anhydrous Na₂SO₄. After filtration, the filtrate was concentrated under reduced pressure. The residue was purified by silica gel column chromatography, eluted with PE:EA (4:1) to afford 8-bromo-2-(methylsulfanyl)pyrido[3,2-d]pyrimidine (1.6 g, 43.11%) as a white solid.

LC-MS: (M+H)⁺ found: 256.0.

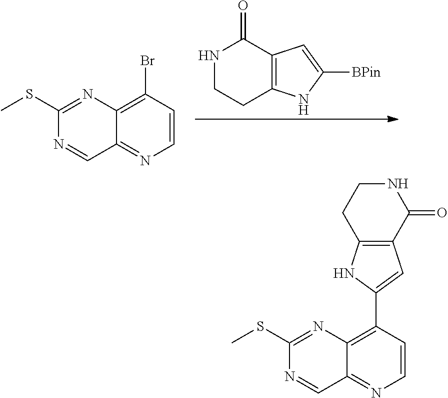

To a stirred solution of 8-bromo-2-(methylsulfanyl)pyrido[3,2-d]pyrimidine (700 mg, 2.73 mmol, 1.00 equiv) and 2-(4,4,5,5-tetramethyl-1,3,2-dioxaborolan-2-yl)-1H,5H,6H,7H-pyrrolo[3,2-c]pyridin-4-one (1075 mg, 4.10 mmol, 1.50 equiv) in dioxane (10 mL) and H₂O (1 mL) were added Na₂CO₃ (869.03 mg, 8.199 mmol, 3.00 equiv) and XPhos Pd G2 (215 mg, 0.27 mmol, 0.10 equiv) dropwise/in portions at it under N₂ atmosphere. The resulting mixture was stirred for 2 h at 80 degrees C. under N₂ atmosphere. Desired product could be detected by LCMS. LC-MS: M+H found: 312. The resulting mixture was concentrated under vacuum. The residue was purified by silica gel column chromatography, eluted with DCM/MeOH=24:1 to afford 2-[2-(methylsulfanyl)pyrido[3,2-d]pyrimidin-8-yl]-1H,5H,6H,7H-pyrrolo[3,2-c]pyridin-4-one (700 mg, 82.26%) as a yellow solid.

LC-MS: (M+H)⁺ found: 312.0.

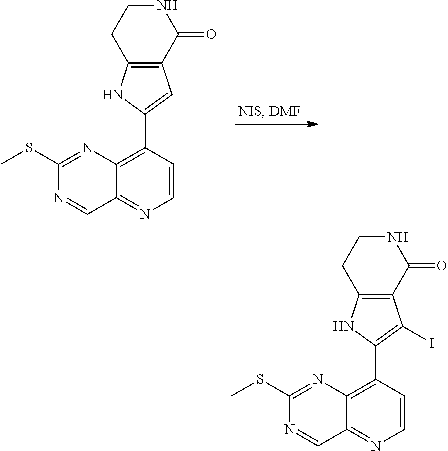 [SMILES: CSc1ncc2nccc(-c3cc4c([nH]3)CCNC4=O)c2n1 ; CSc1ncc2nccc(-c3[nH]c4c(c3I)C(=O)NCC4)c2n1]

To a stirred solution of 2-[2-(methylsulfanyl)pyrido[3,2-d]pyrimidin-8-yl]-1H,5H,6H,7H-pyrrolo[3,2-c]pyridin-4-one (600 mg, 1.93 mmol, 1.00 equiv) and NIS (650 mg, 2.89 mmol, 1.50 equiv) in DMF (10 mL) at rt under N2 atmosphere. The resulting mixture was stirred for overnight at 30 degrees C. under N2 atmosphere. Desired product could be detected by LCMS. LC-MS: M+H found: 438. The reaction was quenched by the addition of Saturated aq. Na₂SO₃ (20 mL) at 0 degrees C. The precipitated solids were collected by filtration and washed with H₂O (20 mL×3). The residue was purified by silica gel column chromatography, eluted with DCM:MeOH=10:1 to afford 3-iodo-2-[2-(methylsulfanyl)pyrido[3,2-d]pyrimidin-8-yl]-1H,5H,6H,7H-pyrrolo[3,2-c]pyridin-4-one (650 mg, 77.14%) as a yellow solid.

LC-MS: (M+H)⁺ found: 438.0.

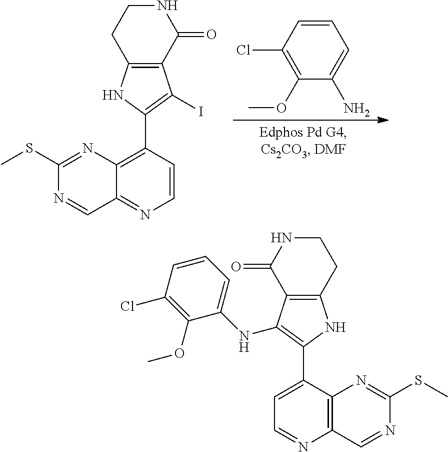

To a stirred solution of 3-iodo-2-[2-(methylsulfanyl)pyrido[3,2-d]pyrimidin-8-yl]-1H,5H,6H,7H-pyrrolo[3,2-c]pyridin-4-one (370 mg, 0.85 mmol, 1.00 equiv) and 3-chloro-2-methoxyaniline (134 mg, 0.85 mmol, 1 equiv) in DMF (4 mL) were added EPhos Pd G4 (78 mg, 0.08 mmol, 0.1 equiv) and Cs₂CO₃ (827 mg, 2.54 mmol, 3 equiv) in portions at rt under Ar atmosphere. The resulting mixture was stirred for 1.5 h at 50 degrees C. under Ar atmosphere. Desired product could be detected by LCMS. LC-MS: M+H found: 467. The resulting mixture was concentrated under vacuum. The residue was purified by silica gel column chromatography, eluted with DCM:MeOH (10:1) to afford 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (220 mg, 55.68%) as a orange solid.

LC-MS: (M+H)⁺ found: 467.0.

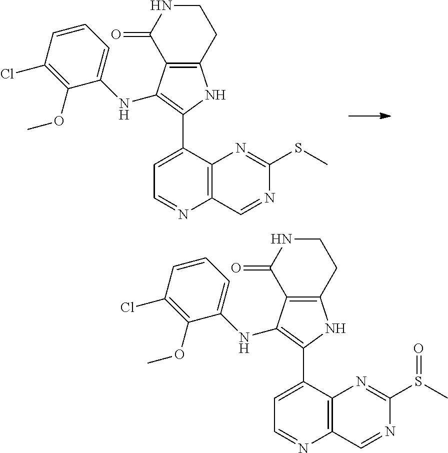

To a stirred solution of 3-[(3-chloro-2-methoxyphenyl)amino]-2-[2-(methylsulfanyl)pyri do[3,2-d]pyrimidin-8-yl]-1H,5H,6H,7H-pyrrolo[3,2-c]pyridin-4-one (50 mg, 0.11 mmol, 1.00 equiv) in DCM (2 mL, 31.46 mmol, 293.80 equiv) were added MCPBA (29 mg, 0.12 mmol, 1.1 equiv) dropwise/in portions at 0 degrees C. under N2 atmosphere. The resulting mixture was stirred for 1 h at 0 degrees C. under N2 atmosphere.

Desired product could be detected by LCMS. LC-MS: M+H found: 483. The resulting mixture was extracted with DCM (3×4 mL). The combined organic layers were dried over anhydrous Na₂SO₄. After filtration, the filtrate was concentrated under reduced pressure. The crude product was used in the next step directly without further purification.

LC-MS: (M+H)⁺ found: 483.0.

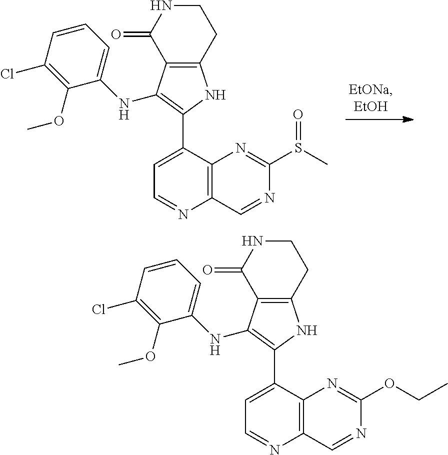

To a stirred solution of 3-[(3-chloro-2-methoxyphenyl)amino]-2-{2-methanesulfinylpyrido[3,2-d]pyrimidin-8-yl}-1H,5H,6H,7H-pyrrolo[3,2-c]pyridin-4-one (90 mg, 0.19 mmol, 1.00 equiv) in CH₃CH₂OH (2.00 mL) were added EtONa (211.36 mg, 0.558 mmol, 3 equiv) dropwise at 0 degrees C. under N2 atmosphere. The resulting mixture was stirred for 1 h at 0 degrees C. under N2 atmosphere. The resulting mixture was concentrated under vacuum and dissolved in DMF. The crude product (50 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 33% B to 63% B in 7 min; Wave Length: 254 nm; RT1 (min): 6.5) to afford 3-[(3-chloro-2-methoxyphenyl)amino]-2-{2-ethoxypyrido[3,2-d]pyrimidin-8-yl}-1H,5H,6H,7H-pyrrolo[3,2-c]pyridin-4-one (14.7 mg) as a yellow solid.

LC-MS: (M+H)⁺ found: 465.0.

¹H NMR (400 MHz, DMSO-d₆) δ 12.00 (s, 1H), 9.50 (s, 1H), 8.70 (d, J=4.8 Hz, 1H), 7.89 (s, 1H), 7.57 (d, J=4.8 Hz, 1H), 7.26 (d, J=2.7 Hz, 1H), 6.73 (m, J=8.1, 1.8 Hz, 1H), 6.68 (t, =8.0 Hz, 1H), 6.17 (m, J=7.8, 1.8 Hz, 1H), 4.65 (m, J=7.0 Hz, 2H), 3.89 (s, 3H), 3.47 (m, J=6.8, 2.5 Hz, 2H), 2.97 (t, J=6.8 Hz, 2H), 1.49 (t, J=7.1 Hz, 3H).

### Example 190. (S)-4-(7-allyl-3-((3-chloro-2-methoxyphenyl)amino)-4-oxo-4,5,6,7-tetrahydro-1H-pyrrolo[3,2-c]pyridin-2-yl)-3-fluoropyridine 1-oxide (compound 828)

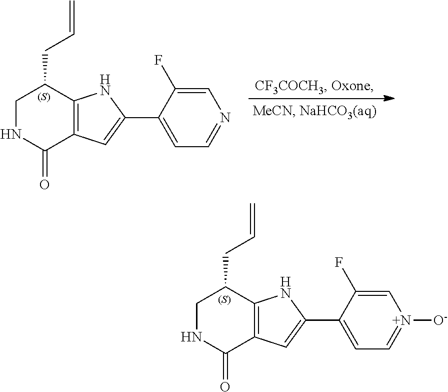

To a stirred solution/mixture of (7S)-2-(3-fluoropyridin-4-yl)-7-(prop-2-en-1-yl)-1H,5H,6H,7H-pyrrolo[3,2-c]pyridin-4-one (300 mg, 1.11 mmol, 1.00 equiv) in H2O (10 mL) were added NaHCO₃ (743 mg, 8.85 mmol, 8 equiv) and oxone (680 mg, 1.11 mmol, 1.00 equiv) in portions at 0 degrees C. under nitrogen atmosphere. The resulting mixture was extracted with EtOAc (3×20 mL). The combined organic layers were washed with brine (3×20 mL), dried over anhydrous Na₂SO₄. After filtration, the filtrate was concentrated under reduced pressure. The residue was purified by silica gel column chromatography, eluted with DCM/MeOH (20:1) to afford (S)-4-(7-allyl-4-oxo-4,5,6,7-tetrahydro-1H-pyrrolo[3,2-c]pyridin-2-yl)-3-fluoropyridine 1-oxide (28 mg, 8.81%) as a yellow solid.

LC-MS: (M+H)⁺ found: 287.95.

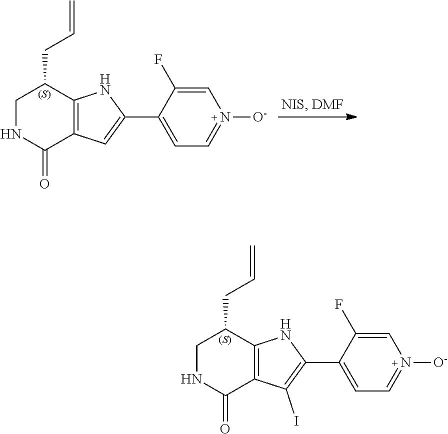

To a stirred mixture of (S)-4-(7-allyl-4-oxo-4,5,6,7-tetrahydro-1H-pyrrolo[3,2-c]pyridin-2-yl)-3-fluoropyridine 1-oxide (28 mg, 0.097 mmol, 1.00 equiv) in DMF (1.00 mL) was added NIS (24 mg, 0.11 mmol, 1.10 equiv) in portions at 0 degrees C. under nitrogen atmosphere. The resulting mixture was stirred for 2 h at room temperature under nitrogen atmosphere. The resulting mixture was concentrated under reduced pressure. The residue was purified by silica gel column chromatography, eluted with DCM/MeOH (20:1) to afford (S)-4-(7-allyl-3-iodo-4-oxo-4,5,6,7-tetrahydro-1H-pyrrolo[3,2-c]pyridin-2-yl)-3-fluoropyridine 1-oxide (26 mg, 64.56%) as a yellow solid.

LC-MS: (M+H)⁺ found: 413.85.

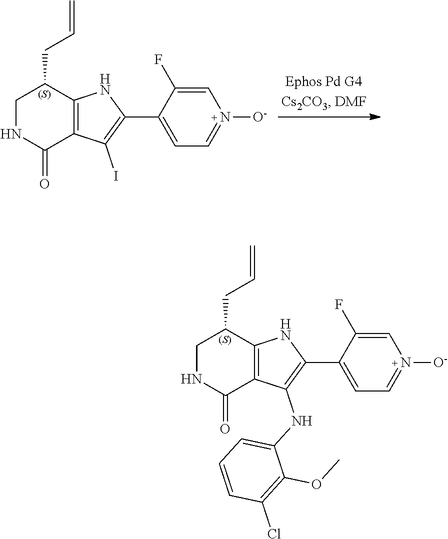

To a stirred mixture of (S)-4-(7-allyl-3-iodo-4-oxo-4,5,6,7-tetrahydro-1H-pyrrolo[3,2-c]pyridin-2-yl)-3-fluoropyridine 1-oxide (40 mg, 0.10 mmol, 1.00 equiv) and 3-chloro-2-methoxyaniline (15 mg, 0.097 mmol, 1.00 equiv) in DMF (1 mL) were added EPhos Pd G4 (44 mg, 0.05 mmol, 0.50 equiv) and Cs₂CO₃ (65 mg, 0.19 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with DCM/MeOH (20:1) to afford (S)-4-(7-allyl-3-((3-chloro-2-methoxyphenyl)amino)-4-oxo-4,5,6,7-tetrahydro-1H-pyrrolo[3,2-c]pyridin-2-yl)-3-fluoropyridine 1-oxide (6.1 mg, 14.23%) as a yellow solid.

LC-MS: (M+H)⁺ found: 443.00

¹H NMR (400 MHz, DMSO-d6) δ 11.77 (s, 1H), 8.50-8.18 (m, 2H), 7.51-7.30 (m, 2H), 7.16 (t, J=2.6 Hz, 1H), 6.92-6.62 (m, 2H), 6.27-6.09 (m, 1H), 5.97-5.78 (m, 1H), 5.23-4.96 (m, 2H), 3.90 (s, 3H), 3.52-3.41 (m, 1H), 3.24-3.14 (m, 1H), 3.11-3.01 (m, 1H), 2.65-2.55 (m, 1H), 2.40-2.26 (m, 1H).

### Example 191. 3-[(3-chloro-2-methoxyphenyl)amino]-2-(3-fluoro-6-methoxy-1,5-naphthyridin-4-yl)-1H,5H,6H,7H-pyrrolo[3,2-c]pyridin-4-one (compound 829)

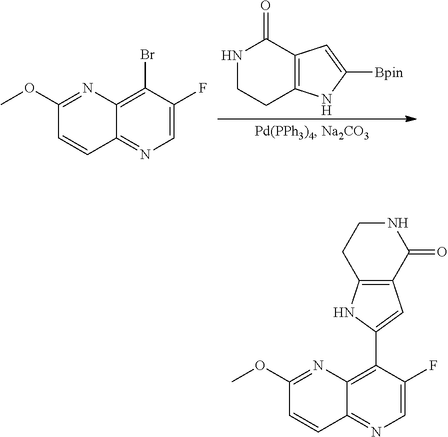

To a stirred solution of 8-bromo-7-fluoro-2-methoxy-1,5-naphthyridine (200 mg, 0.78 mmol, 1.00 equiv) and 2-(4,4,5,5-tetramethyl-1,3,2-dioxaborolan-2-yl)-1H,5H,6H,7H-pyrrolo[3,2-c]pyridin-4-one (306 mg, 1.167 mmol, 1.5 equiv) in dioxane (5 mL) and H₂O (0.5 mL) were added 2nd Generation XPhos Precatalyst/X-Phos aminobiphenyl palladium chloride precatalyst (62 mg, 0.08 mmol, 0.1 equiv) and Na₂CO₃ (247 mg, 2.33 mmol, 3 equiv) dropwise/in portions at room temperature under nitrogen atmosphere. The resulting mixture was stirred for 2 h at 80 degrees C. under nitrogen atmosphere. Desired product could be detected by LCMS. LC-MS: M+H found: 313. The resulting mixture was concentrated under reduced pressure. The residue was purified by silica gel column chromatography, eluted with CH₂Cl₂/MeOH (10:1) to afford 2-(3-fluoro-6-methoxy-1,5-naphthyridin-4-yl)-1H,5H,6H,7H-pyrrolo[3,2-c]pyridin-4-one (100 mg, 41.16%) as a yellow solid.

LC-MS: M+H found: 313.0.

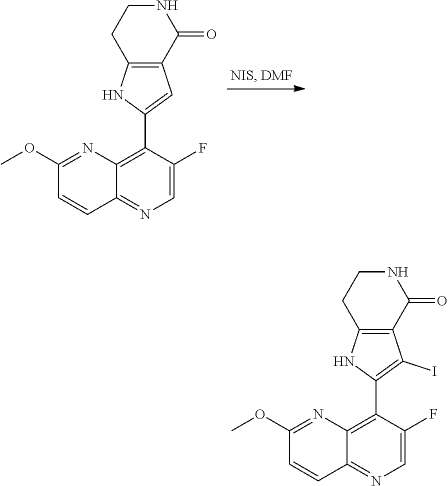 [SMILES: COc1ccc2ncc(F)c(-c3cc4c([nH]3)CCNC4=O)c2n1 ; COc1ccc2ncc(F)c(-c3[nH]c4c(c3I)C(=O)NCC4)c2n1]

To a stirred solution of 2-(3-fluoro-6-methoxy-1,5-naphthyridin-4-yl)-1H,5H,6H,7H-pyrrolo[3,2-c]pyridin-4-one (90 mg, 0.29 mmol, 1.00 equiv) in DMF (4.5 mL) was added NIS (130 mg, 0.58 mmol, 2 equiv) in portions at 50 degrees C. under N2 atmosphere.

Desired product could be detected by LCMS. LC-MS: M+H found: 439. The resulting mixture was extracted with DCM:MeOH=10:1 (3×20 ml). The combined organic layers were washed with NaCl (3×100 ml), dried over anhydrous Na₂SO₄. After filtration, the filtrate was concentrated under reduced pressure. The residue was purified by silica gel column chromatography, eluted with DCM:MeOH=40:1 to afford 2-(3-fluoro-6-methoxy-1,5-naphthyridin-4-yl)-3-iodo-1H,5H,6H,7H-pyrrolo[3,2-c]pyridin-4-one (70 mg, 55.43%) as a yellow solid.

LC-MS: (M+H)⁺ found: 438.85.

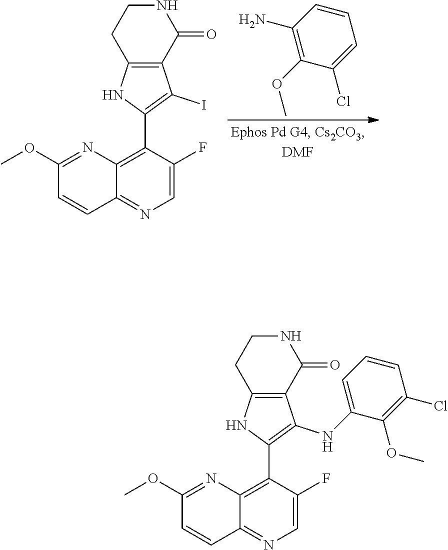

To a stirred solution of 2-(3-fluoro-6-methoxy-1,5-naphthyridin-4-yl)-3-iodo-1H,5H,6H,7H-pyrrolo[3,2-c]pyridin-4-one (60 mg, 0.14 mmol, 1.00 equiv) and 3-chloro-2-methoxyaniline (22 mg, 0.14 mmol, 1 equiv) in DMF (1 mL) were added EPhos Pd G4 (12 mg, 0.014 mmol, 0.1 equiv) and Cs₂CO₃ (133 mg, 0.41 mmol, 3 equiv) in portions at rt under Ar atmosphere. The resulting mixture was stirred for 2 h at 50 degrees C. under Ar atmosphere. The resulting mixture was concentrated under reduced pressure. The resulting mixture was concentrated under vacuum and dissolved in DMF. The crude product (100 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 32% B to 62% B in 7 min; Wave Length: 254 nm; RT1 (min): 6.5) to afford 3-[(3-chloro-2-methoxyphenyl)amino]-2-(3-fluoro-6-methoxy-1,5-naphthyridin-4-yl)-1H,5H,6H,7H-pyrrolo[3,2-c]pyridin-4-one (19.4 mg, 30.28%) as a yellow solid.

LC-MS: (M+H)⁺ found: 438.85.

¹H NMR (400 MHz, DMSO-d₆) δ 11.58 (s, 1H), 8.73 (d, J=1.6 Hz, 1H), 8.34 (d, J=9.1 Hz, 1H), 7.78 (s, 1H), 7.32 (d, J=9.0 Hz, 1H), 7.14 (d, J=2.7 Hz, 1H), 6.55 (dd, J=8.0, 1.5 Hz, 1H), 6.46 (t, J=8.1 Hz, 1H), 6.14 (dd, J=8.2, 1.5 Hz, 1H), 4.08 (s, 3H), 3.67 (s, 3H), 3.48 (td, J=6.9, 2.5 Hz, 2H), 2.91 (t, J=6.8 Hz, 2H).

### Example 192. 3-[(3-chloro-2-methoxyphenyl)amino]-2-[2-(difluoromethyl)pyrimidin-4-yl]-1H,5H,6H,7H-pyrrolo[3,2-c]pyridin-4-one (compound 472)

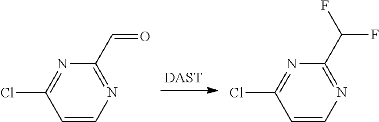

A mixture of 4-chloropyrimidine-2-carbaldehyde (100 mg, 0.702 mmol, 1 equiv) and DAST (226.17 mg, 1.404 mmol, 2 equiv) in DCM (7 mL) was stirred for 2 h at −78° C. under nitrogen atmosphere. Desired product could be detected by TLC. The resulting mixture was concentrated under vacuum. The residue was purified by silica gel column chromatography, eluted with PE/EA (8:1) to afford 4-chloro-2-(difluoromethyl)pyrimidine (110 mg, 95.29%) as a yellow oil.

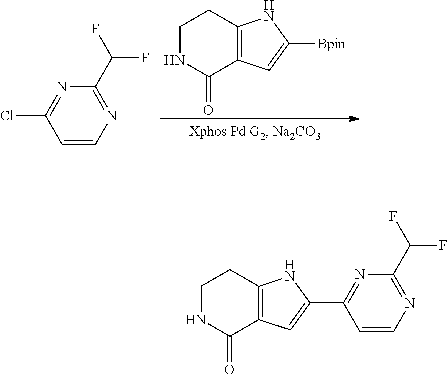

To a stirred mixture of 4-chloro-2-(difluoromethyl)pyrimidine (500 mg, 3.039 mmol, 1 equiv) and 2-(4,4,5,5-tetramethyl-1,3,2-dioxaborolan-2-yl)-1H,5H,6H,7H-pyrrolo[3,2-c]pyridin-4-one (955.83 mg, 3.647 mmol, 1.2 equiv) in 1,4-dioxane (15 mL) and water (3 mL) were added Na2CO3 (644.15 mg, 6.078 mmol, 2 equiv) and XPhos palladium(II) biphenyl-2-amine chloride (239.09 mg, 0.304 mmol, 0.1 equiv) in portions at room temperature under argon atmosphere. The resulting mixture was stirred for overnight at 50° C. under argon atmosphere. Desired product could be detected by LCMS. The resulting mixture was concentrated under vacuum. The residue was purified by silica gel column chromatography, eluted with CH2Cl2/MeOH (20:1) to afford 2-[2-(difluoromethyl)pyrimidin-4-yl]-1H,5H,6H,7H-pyrrolo[3,2-c]pyridin-4-one (550 mg, 68.50%) as a yellow solid.

LC-MS: [M+H]⁺ found: 264.95.

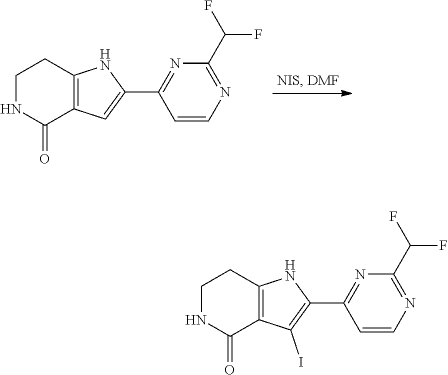 [SMILES: O=C1NCCc2[nH]c(-c3ccnc(C(F)F)n3)cc21 ; O=C1NCCc2[nH]c(-c3ccnc(C(F)F)n3)c(I)c21]

A mixture of 2-[2-(difluoromethyl)pyrimidin-4-yl]-1H,5H,6H,7H-pyrrolo[3,2-c]pyridin-4-one (133 mg, 0.50 mmol, 1 equiv) and NIS (135 mg, 0.60 mmol, 1.2 equiv) in DMF (2 mL) was stirred for 1 h at room temperature under air atmosphere. Desired product could be detected by LCMS. The reaction was quenched by the addition of sat. sodium sulfite (aq.) (0.5 mL) at room temperature. The resulting mixture was concentrated under reduced pressure. The residue was purified by silica gel column chromatography, eluted with CH2Cl2/MeOH (15:1) to afford 2-[2-(difluoromethyl)pyrimidin-4-yl]-3-iodo-1H,5H,6H,7H-pyrrolo[3,2-c]pyridin-4-one (90 mg, 45.83%) as a yellow solid.

LC-MS: [M+H]⁺ found: 390.85.

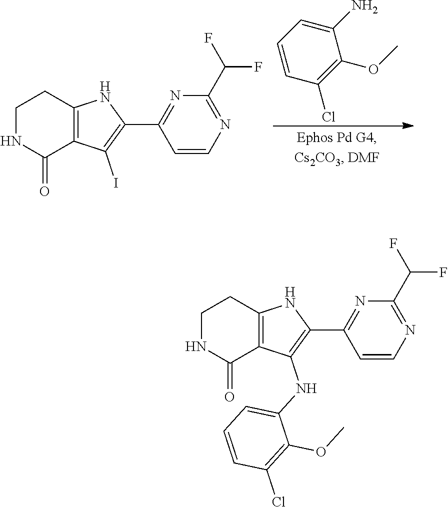

To a stirred mixture of 2-[2-(difluoromethyl)pyrimidin-4-yl]-3-iodo-1H,5H,6H,7H-pyrrolo[3,2-c]pyridin-4-one (80 mg, 0.20 mmol, 1.00 equiv) and 3-chloro-2-methoxyaniline (29 mg, 0.184 mmol, 0.9 equiv) in DMF (2 mL) were added EPhos Pd G4 (18 mg, 0.021 mmol, 0.1 equiv) and Cs₂CO₃ (133 mg, 0.410 mmol, 2.0 equiv) in portions at room temperature under argon atmosphere. The resulting mixture was stirred for 2 h at 50° C. under argon atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 38% B to 54% B in 8 min, 54% B; Wave Length: 254/220 nm; RT1 (min): 7) to afford 3-[(3-chloro-2-methoxyphenyl)amino]-2-[2-(difluoromethyl)pyrimidin-4-yl]-1H,5H,6H,7H-pyrrolo[3,2-c]pyridin-4-one (24.4 mg, 27.63%) as a yellow solid.

LC-MS: [M+H]⁺ found: 420.00.

¹H NMR (300 MHz, DMSO-d6) δ 12.07 (s, 1H), 8.67 (d, J=5.4 Hz, 1H), 8.02 (s, 1H), 7.25 (t, J=7.8 Hz, 2H), 6.82 (t, J=3.6 Hz, 3H), 6.34 (t, J=4.8 Hz, 1H), 3.92 (s, 3H), 3.44-3.40 (m, 2H), 2.91 (t, J=6.6 Hz, 2H).

### Example 193. 3-[(3-chloro-2-methoxyphenyl)amino]-2-[2-(trifluoromethyl)pyrimidin-4-yl]-1H,5H,6H,7H-pyrrolo[3,2-c]pyridin-4-one (compound 476)

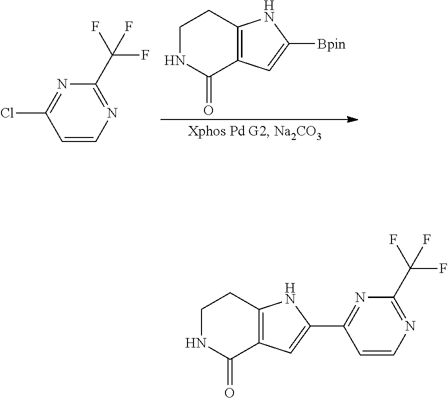

To a stirred mixture of 2-(4,4,5,5-tetramethyl-1,3,2-dioxaborolan-2-yl)-1H,5H,6H,7H-pyrrolo[3,2-c]pyridin-4-one (345 mg, 1.31 mmol, 2.00 equiv) and 4-chloro-2-(trifluoromethyl)pyrimidine (120 mg, 0.66 mmol, 1.00 equiv) in dioxane (3.60 mL) and H₂O (0.36 mL) was added XPhos Pd G2 (52 mg, 0.07 mmol, 0.10 equiv) dropwise at room temperature. The resulting mixture was stirred for 2 h at 50 degrees C. under nitrogen atmosphere. The resulting mixture was concentrated under reduced pressure. The residue was purified by silica gel column chromatography, eluted with DCM/MeOH (20:1) to afford 2-[2-(trifluoromethyl)pyrimidin-4-yl]-1H,5H,6H,7H-pyrrolo[3,2-c]pyridin-4-one (160 mg, 86.23%) as a yellow solid.

LC-MS: (M+H)⁺ found: 282.90.

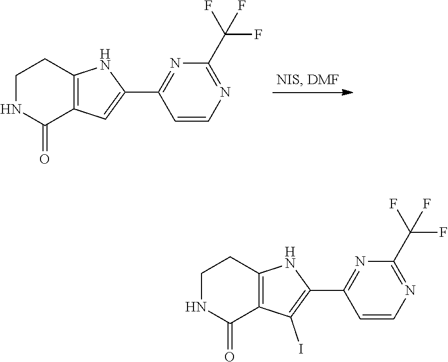 [SMILES: O=C1NCCc2[nH]c(-c3ccnc(C(F)(F)F)n3)cc21 ; O=C1NCCc2[nH]c(-c3ccnc(C(F)(F)F)n3)c(I)c21]

To a stirred mixture of 2-[2-(trifluoromethyl)pyrimidin-4-yl]-1H,5H,6H,7H-pyrrolo[3,2-c]pyridin-4-one (170 mg, 0.60 mmol, 1.00 equiv) in DMF (3 mL) was added NIS (271 mg, 1.2 mmol, 2.00 equiv) dropwise at 0 degrees C. The resulting mixture was stirred for overnight at room temperature under nitrogen atmosphere. The reaction was quenched with sat. Na₂SO₃ (aq.) at 0 degrees C. The resulting mixture was concentrated under reduced pressure. The residue was purified by silica gel column chromatography, eluted with DCM/MeOH (20:1) to afford 3-iodo-2-[2-(trifluoromethyl)pyrimidin-4-yl]-1H,5H,6H,7H-pyrrolo[3,2-c]pyridin-4-one (200 mg, 81.36%) as a light yellow solid.

LC-MS: (M+H)⁺ found: 408.85.

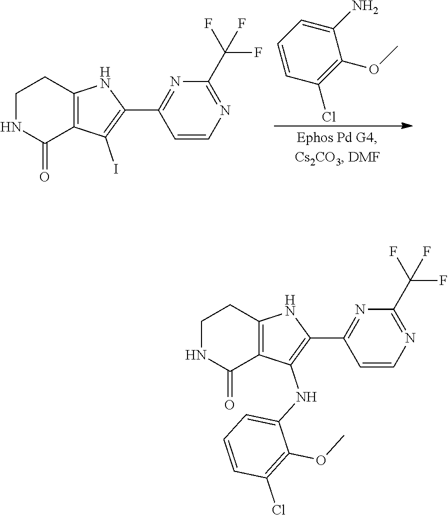

To a stirred mixture of 3-iodo-2-[2-(trifluoromethyl)pyrimidin-4-yl]-1H,5H,6H,7H-pyrrolo[3,2-c]pyridin-4-one (100 mg, 0.25 mmol, 1.00 equiv) and 3-chloro-2-methoxyaniline (39 mg, 0.25 mmol, 1.00 equiv) in DMF (5 mL) were added EPhos Pd G4 (45 mg, 0.05 mmol, 0.20 equiv) and Cs2CO3 (160 mg, 0.49 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with DCM/MeOH (20:1) to afford 3-[(3-chloro-2-methoxyphenyl)amino]-2-[2-(trifluoromethyl)pyrimidin-4-yl]-1H,5H,6H,7H-pyrrolo[3,2-c]pyridin-4-one (64.8 mg, 60.41%) as a white solid.

LC-MS: (M+H)⁺ found: 438.20.

¹H NMR (400 MHz, DMSO-d6) δ 12.09 (s, 1H), 8.73 (d, J=5.6 Hz, 1H), 8.03 (s, 1H), 7.34 (d, J=5.9 Hz, 2H), 6.88-6.78 (m, 2H), 6.35 (m, 1H), 3.91 (s, 3H), 3.44 (m, 2H), 2.92 (t, J=6.7 Hz, 2H).

### Example 194. 3-[(3-chloro-2-methoxyphenyl)amino]-2-(7-fluoro-6-methoxy-1,5-naphthyridin-4-yl)-1H,5H,6H,7H-pyrrolo[3,2-c]pyridin-4-one (compound 477)

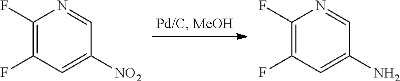 [SMILES: O=[N+]([O-])c1cnc(F)c(F)c1 ; Nc1cnc(F)c(F)c1]

To a solution of 2,3-difluoro-5-nitropyridine (2.5 g, 15.62 mmol, 1.00 equiv) in MeOH (160 mL) was added Pd/C (10%, 300 mg) under nitrogen atmosphere. The mixture was hydrogenated at room temperature for 2.5 h under hydrogen atmosphere using a hydrogen balloon, filtered through a Celite pad and concentrated under reduced pressure. Desired product could be detected by LCMS. LC-MS: M+H found: 131. The resulting mixture was filtered, the filter cake was washed with MeOH (3×50 ml). The filtrate was concentrated under reduced pressure to afford 5,6-difluoropyridin-3-amine (2 g, 98.44%) as a brown solid.

LC-MS: M+H found: 131.0.

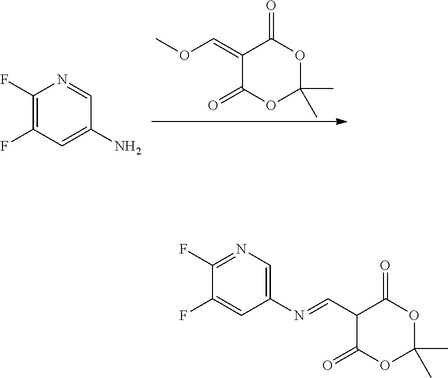

To a stirred solution of 5,6-difluoropyridin-3-amine (1.93 g, 14.84 mmol, 1.00 equiv) and 5-(methoxymethylidene)-2,2-dimethyl-1,3-dioxane-4,6-dione (3.31 g, 17.80 mmol, 1.2 equiv) in DMF (40 mL) at rt under N2 atmosphere. The resulting mixture was stirred for 1 h at 80 degrees C. under N2 atmosphere. Desired product could be detected by LCMS.

LC-MS: M−H found: 283. The precipitated solids were collected by filtration and washed with MeOH (3×100 ml) to afford 5-[(1E)-[(5,6-difluoropyridin-3-yl)imino]methyl]-2,2-dimethyl-1,3-dioxane-4,6-dione (3.3 g, 78.27%) as a yellow solid.

LC-MS: (M+H)⁺ found: 283.0.

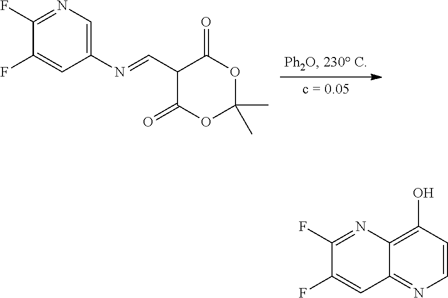 [SMILES: CC1(C)OC(=O)C(/C=N/c2cnc(F)c(F)c2)C(=O)O1 ; Oc1ccnc2cc(F)c(F)nc12]

To a stirred phenoxybenzene (210 mL) at rt to 230 degrees C. under N2 atmosphere. To the above solution was added 5-[(1E)-[(5,6-difluoropyridin-3-yl)imino]methyl]-2,2-dimethyl-1,3-dioxane-4,6-dione (3 g, 10.55 mmol, 1.00 equiv) in portions at 230 degrees C. The resulting mixture was stirred for additional 5 min at 230 degrees C. Desired product could be detected by LCMS. LC-MS: M+H found: 183. The above mixture was added to 15 ml n-Hexane dropwise, The precipitated solids were collected by filtration and washed with n-Hexane (3×100 ml) to afford 6,7-difluoro-1,5-naphthyridin-4-ol (1.6 g, 83.23%) as a white solid.

LC-MS: (M+H)⁺ found: 183.0.

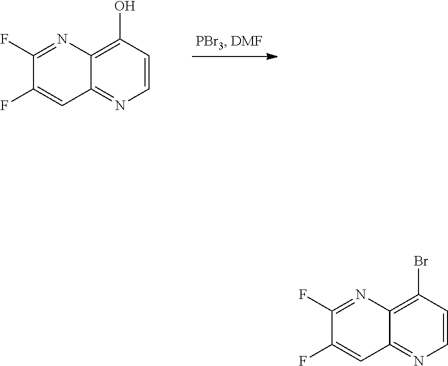 [SMILES: Oc1ccnc2cc(F)c(F)nc12 ; Fc1cc2nccc(Br)c2nc1F]

To a stirred solution of 6,7-difluoro-1,5-naphthyridin-4-ol (1.66 g, 9.11 mmol, 1.00 equiv) in DMF (30 mL) was added PBr₃ (3.70 g, 13.67 mmol, 1.5 equiv) dropwise at 0 degrees C. under N2 atmosphere. The resulting mixture was stirred for 1 h at rt under N2 atmosphere.

Desired product could be detected by LCMS. LC-MS: M+H found: 245. The resulting mixture was diluted with Na₂CO₃ aq (30 ml). The resulting mixture was extracted with EA (3×80 mL). The combined organic layers were washed with NaCl (3×100 ml), dried over anhydrous Na₂SO₄. After filtration, the filtrate was concentrated under reduced pressure. The residue was purified by silica gel column chromatography, eluted with PE:EA (3:2) to afford 8-bromo-2,3-difluoro-1,5-naphthyridine (2.1 g, 94.03%) as a yellow solid.

LC-MS: (M+H)⁺ found: 245.0.

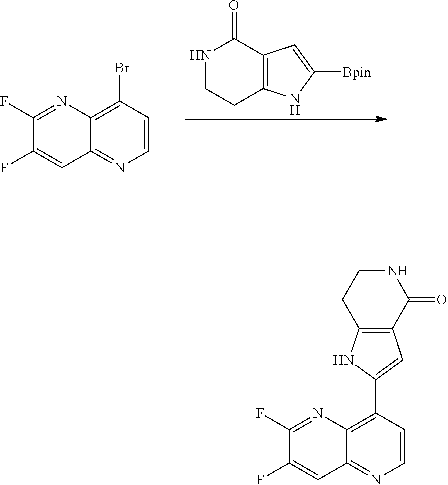

To a stirred solution of 8-bromo-2,3-difluoro-1,5-naphthyridine (500 mg, 2.04 mmol, 1.00 equiv) and 2-(4,4,5,5-tetramethyl-1,3,2-dioxaborolan-2-yl)-1H,5H,6H,7H-pyrrolo[3,2-c]pyridin-4-one (641.86 mg, 2.449 mmol, 1.2 equiv) in dioxane (10 mL) and H₂O (1 mL) were added 2nd Generation XPhos Precatalyst (161 mg, 0.20 mmol, 0.1 equiv) and Na₂CO₃ (649 mg, 6.12 mmol, 3 equiv) dropwise/in portions at rt under N2 atmosphere. The resulting mixture was stirred for 1.5 h at 80 degrees C. under N2 atmosphere. Desired product could be detected by LCMS. LC-MS: M+H found: 301. The resulting mixture was concentrated under reduced pressure. The residue was purified by silica gel column chromatography, eluted with DCM:MeOH (95:5) to afford 2-(6,7-difluoro-1,5-naphthyridin-4-yl)-1H,5H,6H,7H-pyrrolo[3,2-c]pyridin-4-one (180 mg, 29.38%) as a yellow solid.

LC-MS: (M+H)⁺ found: 301.0.

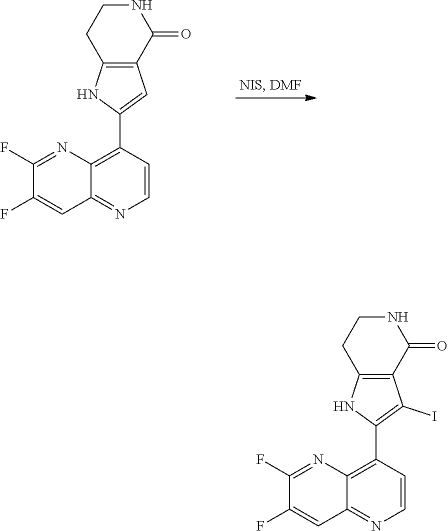 [SMILES: O=C1NCCc2[nH]c(-c3ccnc4cc(F)c(F)nc34)cc21 ; O=C1NCCc2[nH]c(-c3ccnc4cc(F)c(F)nc34)c(I)c21]

To a stirred solution/mixture of 2-(6,7-difluoro-1,5-naphthyridin-4-yl)-1H,5H,6H,7H-pyrrolo[3,2-c]pyridin-4-one (180 mg, 0.60 mmol, 1.00 equiv) and NIS (202 mg, 0.90 mmol, 1.5 equiv) in DMF (3 mL) at RT under N2 atmosphere. The resulting mixture was stirred for 3 H at RT under N2 atmosphere. Desired product could be detected by LCMS.

LC-MS: M+H found: 427. The resulting mixture was concentrated under vacuum. The residue was purified by silica gel column chromatography, eluted with DCM:MeOH (10:1) to afford 2-(6,7-difluoro-1,5-naphthyridin-4-yl)-3-iodo-1H,5H,6H,7H-pyrrolo[3,2-c]pyridin-4-one (180 mg, 70.46%) as a yellow solid.

LC-MS: (M+H)⁺ found: 427.0.

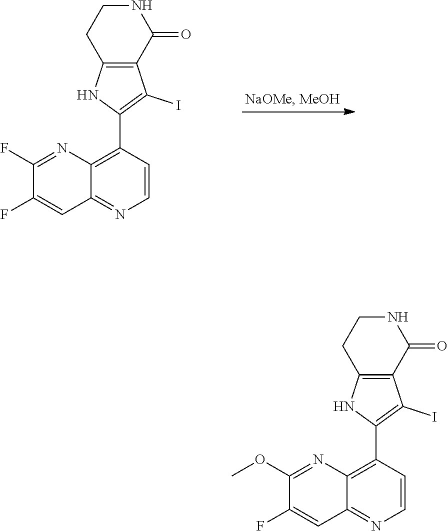 [SMILES: O=C1NCCc2[nH]c(-c3ccnc4cc(F)c(F)nc34)c(I)c21 ; COc1nc2c(-c3[nH]c4c(c3I)C(=O)NCC4)ccnc2cc1F]

To a stirred solution of 2-(6,7-difluoro-1,5-naphthyridin-4-yl)-3-iodo-1H,5H,6H,7H-pyrrolo[3,2-c]pyridin-4-one (110 mg, 0.258 mmol, 1.00 equiv) in MeOH (2.5 mL, 61.747 mmol) was added NaOMe (27.89 mg, 0.516 mmol, 2 equiv) dropwise at 0 degrees C. under N₂ atmosphere. The resulting mixture was stirred for 2 h at rt under N₂ atmosphere. Desired product could be detected by LCMS. LC-MS: M+H found: 439. The resulting mixture was concentrated under vacuum. The residue was purified by silica gel column chromatography, eluted with DCM:MeOH (10:1) to afford 2-(7-fluoro-6-methoxy-1,5-naphthyridin-4-yl)-3-iodo-1H,5H,6H,7H-pyrrolo[3,2-c]pyridin-4-one (70 mg, 61.89%) as a yellow solid.

LC-MS: (M+H)⁺ found: 439.0.

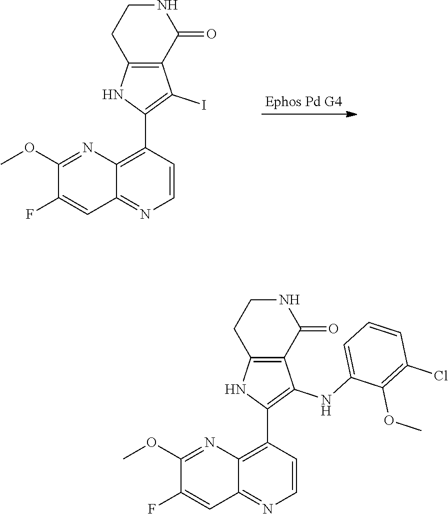 [SMILES: COc1nc2c(-c3[nH]c4c(c3I)C(=O)NCC4)ccnc2cc1F ; COc1nc2c(-c3[nH]c4c(c3Nc3cccc(Cl)c3OC)C(=O)NCC4)ccnc2cc1F]

To a stirred solution of 2-(7-fluoro-6-methoxy-1,5-naphthyridin-4-yl)-3-iodo-1H,5H,6H,7H-pyrrolo[3,2-c]pyridin-4-one (65 mg, 0.15 mmol, 1.00 equiv) and 3-chloro-2-methoxyaniline (24 mg, 0.15 mmol, 1 equiv) in DMF (1.5 mL) were added Ephos Pd G4 (14 mg, 0.01 mmol, 0.1 equiv) and Cs₂CO₃ (145 mg, 0.44 mmol, 3 equiv) in portions at rt under Ar atmosphere. The resulting mixture was stirred for 1.5 h at 50 degrees C. under Ar atmosphere. The resulting mixture was concentrated under reduced pressure. The resulting mixture was concentrated under vacuum and dissolved in DMF. The crude product (60 mg) was purified by Prep-HPLC with the following conditions (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 44% B to 53% B in 10 min, 53% B; Wave Length: 220/254 nm; RT1 (min): 8.23) to afford 3-[(3-chloro-2-methoxyphenyl)amino]-2-(7-fluoro-6-methoxy-1,5-naphthyridin-4-yl)-1H,5H,6H,7H-pyrrolo[3,2-c]pyridin-4-one (14.4 mg, 20.75%) as a yellow solid.

LC-MS: (M+H)⁺ found: 468.0.

¹H NMR (400 MHz, DMSO-d₆) δ 11.87 (s, 1H), 8.61 (d, J=4.8 Hz, 1H), 8.24 (d, J=10.9 Hz, 1H), 7.79 (s, 1H), 7.51 (d, J=4.8 Hz, 1H), 7.23 (d, J=2.6 Hz, 1H), 6.69 (dd, J=8.1, 1.7 Hz, 1H), 6.64 (t, J=8.0 Hz, 1H), 6.16 (m, J=7.9, 1.8 Hz, 1H), 4.27 (s, 3H), 3.87 (s, 3H), 3.47 (m, J=6.8, 2.5 Hz, 2H), 2.95 (t, J=6.8 Hz, 2H).

### Example 195. (7R)-3-[(3-chloro-2-methoxyphenyl)amino]-7-[2-(dimethylamino)ethyl]-2-(3-fluoropyridin-4-yl)-1H,5H,6H,7H-pyrrolo[3,2-c]pyridin-4-one (compound 486)

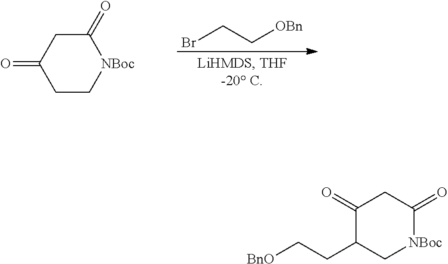 [SMILES: CC(C)(C)OC(=O)N1CCC(=O)CC1=O ; BrCCOCc1ccccc1 ; CC(C)(C)OC(=O)N1CC(CCOCc2ccccc2)C(=O)CC1=O]

To a stirred mixture of tert-butyl 2,4-dioxopiperidine-1-carboxylate (20 g, 93.794 mmol, 1.00 equiv) and [(2-bromoethoxy)methyl]benzene (80.70 g, 375.192 mmol, 4.00 equiv) in THF (600 mL) was added LiHMDS (235.00 mL, 235.000 mmol, 2.51 equiv) dropwise at −20 degrees C. under argon atmosphere. The resulting mixture was stirred for 1 h at −20 degrees C. under argon atmosphere. The reaction was quenched with Water at −20 degrees C. The mixture was acidified to pH=5 with 5% KHSO4 solution. The resulting mixture was extracted with CH2Cl2. The combined organic layers were washed with brine, dried over anhydrous Na2SO4. After filtration, the filtrate was concentrated under reduced pressure. The residue was purified by silica gel column chromatography, eluted with PE/EtOAc (2:1) to afford tert-butyl 5-[2-(benzyloxy)ethyl]-2,4-dioxopiperidine-1-carboxylate (17 g, 52.17%) as a green oil.

LC-MS: M+H found: 348.

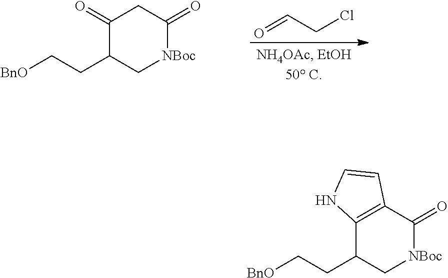

Into a round-bottom flask were added tert-butyl 5-[2-(benzyloxy)ethyl]-2,4-dioxopiperidine-1-carboxylate (10 g, 28.78 mmol, 1.00 equiv), Ammonia, 7.0 M Solution In Methanol, SpcSeal (10 mL), NH₄OAc (11.09 g, 143.92 mmol, 5.0 equiv), EtOH (100 mL) and chloroacetaldehyde (2.71 g, 34.54 mmol, 1.2 equiv) at room temperature. The resulting mixture was stirred for 1 h at 50 degrees C. under nitrogen atmosphere. The solvents were concentrated under reduced pressure. The reaction was quenched with Water at room temperature. The resulting mixture was extracted with EtOAc (3×200 mL). The combined organic layers were washed with brine (1×300 mL), dried over anhydrous Na2SO4. After filtration, the filtrate was concentrated under reduced pressure. The residue was purified by silica gel column chromatography, eluted with PE/EA (2:1) to afford tert-butyl 7-[2-(benzyloxy)ethyl]-4-oxo-1H,6H,7H-pyrrolo[3,2-c]pyridine-5-carboxylate (1.45 g, 13.60%) as a white solid.

LC-MS: M+H found: 371.

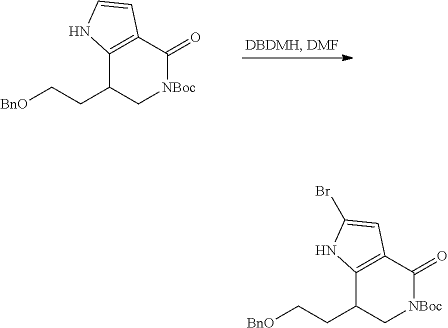 [SMILES: CC(C)(C)OC(=O)N1CC(CCOCc2ccccc2)c2[nH]ccc2C1=O ; CC(C)(C)OC(=O)N1CC(CCOCc2ccccc2)c2[nH]c(Br)cc2C1=O]

To a stirred solution of tert-butyl 7-[2-(benzyloxy)ethyl]-4-oxo-1H,6H,7H-pyrrolo[3,2-c]pyridine-5-carboxylate (1.05 g, 2.83 mmol, 1.00 equiv) in DMF (10 mL) was added dbdmh (0.39 g, 1.36 mmol, 0.48 equiv) in portions at −50 degrees C. under nitrogen atmosphere. The reaction was monitored by LCMS. Desired product could be detected by LCMS. The resulting mixture was diluted with water (10 mL). The resulting mixture was extracted with EtOAc (3×20 mL). The combined organic layers were washed with brine (3×10 mL), dried over anhydrous Na2SO4. After filtration, the filtrate was concentrated under reduced pressure. The residue was purified by silica gel column chromatography, eluted with PE/EA (5:1) to afford tert-butyl 7-[2-(benzyloxy)ethyl]-2-bromo-4-oxo-1H,6H,7H-pyrrolo[3,2-c]pyridine-5-carboxylate (1200 mg, 83.86%) as a white oil.

LC-MS: M+H found: 449.0.

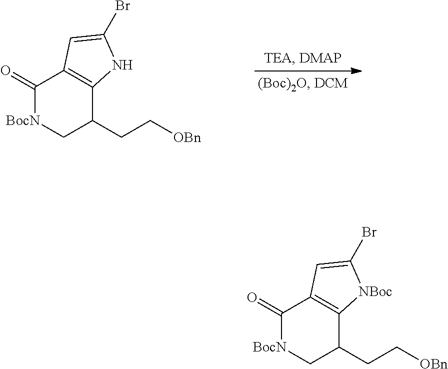 [SMILES: CC(C)(C)OC(=O)N1CC(CCOCc2ccccc2)c2[nH]c(Br)cc2C1=O ; CC(C)(C)OC(=O)N1CC(CCOCc2ccccc2)c2c(cc(Br)n2C(=O)OC(C)(C)C)C1=O]

A solution of tert-butyl 7-[2-(benzyloxy)ethyl]-2-bromo-4-oxo-1H,6H,7H-pyrrolo[3,2-c]pyridine-5-carboxylate (500 mg, 1.11 mmol, 1.00 equiv) and (Boc)₂O (267 mg, 1.224 mmol, 1.1 equiv) and TEA (135 mg, 1.34 mmol, 1.2 equiv) in DCM (5.00 mL) was stirred for 2 h at room temperature under nitrogen atmosphere. The reaction was monitored by LCMS. Desired product could be detected by LCMS. The resulting mixture was concentrated under reduced pressure. The residue was purified by Prep-TLC (PE/EA 5:1) to afford 1,5-di-tert-butyl 7-[2-(benzyloxy)ethyl]-2-bromo-4-oxo-6H,7H-pyrrolo[3,2-c]pyridine-1,5-dicarboxylate (590 mg, 92.64%) as a white oil.

LC-MS: M+H found: 549.0.

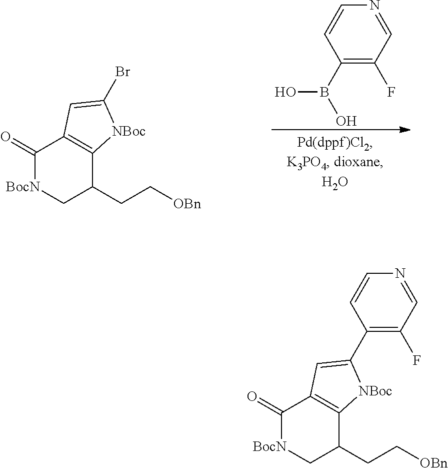 [SMILES: CC(C)(C)OC(=O)N1CC(CCOCc2ccccc2)c2c(cc(Br)n2C(=O)OC(C)(C)C)C1=O ; OB(O)c1ccncc1F ; CC(C)(C)OC(=O)N1CC(CCOCc2ccccc2)c2c(cc(-c3ccncc3F)n2C(=O)OC(C)(C)C)C1=O]

A solution of 1,5-di-tert-butyl 7-[2-(benzyloxy)ethyl]-2-bromo-4-oxo-6H,7H-pyrrolo[3,2-c]pyridine-1,5-dicarboxylate (6.5 g, 11.83 mmol, 1.00 equiv) and 3-fluoropyridin-4-ylboronic acid (2.50 g, 17.74 mmol, 1.5 equiv) and Pd(dppf)Cl₂·CH₂Cl₂ (964 mg, 1.18 mmol, 0.1 equiv) and K₃PO₄ (7.53 g, 35.49 mmol, 3 equiv) in dioxane (100 mL) and H2O (20 mL) was stirred for 1 h at 50 degrees C. under nitrogen atmosphere. The reaction was monitored by LCMS. Desired product could be detected by LCMS. The resulting mixture was concentrated under vacuum. The aqueous layer was extracted with EtOAc (4×50 mL). The residue was purified by silica gel column chromatography, eluted with PE/EA (10:1) to afford 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (5.6 g, 82.02%) as a yellow oil.

LC-MS: M+H found: 566.0.

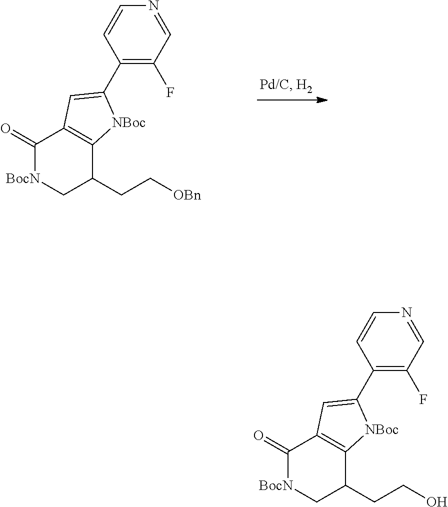 [SMILES: CC(C)(C)OC(=O)N1CC(CCOCc2ccccc2)c2c(cc(-c3ccncc3F)n2C(=O)OC(C)(C)C)C1=O ; CC(C)(C)OC(=O)N1CC(CCO)c2c(cc(-c3ccncc3F)n2C(=O)OC(C)(C)C)C1=O]

To a solution of 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (4 g, 7.072 mmol, 1.00 equiv) in 140 mL MeOH was added dry Pd/C (10%, 1.6 g) under nitrogen atmosphere in a 500 mL round-bottom flask. The mixture was hydrogenated at room temperature for 2 days under hydrogen atmosphere using a hydrogen balloon, filtered through a Celite pad and concentrated under reduced pressure. The reaction was monitored by LCMS. Desired product could be detected by LCMS. The resulting mixture was filtered, the filter cake was washed with MeOH (3×50 mL). The filtrate was concentrated under reduced pressure to afford 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (2.27 g, 57.38%) as a white oil.

LC-MS: M+H found: 476.0.

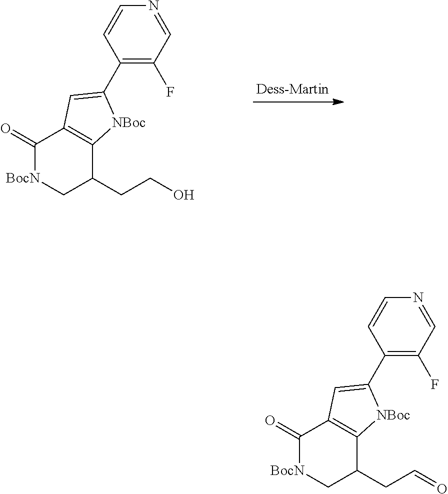 [SMILES: CC(C)(C)OC(=O)N1CC(CCO)c2c(cc(-c3ccncc3F)n2C(=O)OC(C)(C)C)C1=O ; CC(C)(C)OC(=O)N1CC(CC=O)c2c(cc(-c3ccncc3F)n2C(=O)OC(C)(C)C)C1=O]

To a stirred solution of 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (500 mg, 1.05 mmol, 1.00 equiv) in DCM (10 mL) were added Dess-Martin (535 mg, 1.26 mmol, 1.20 equiv) in portions at 0 degrees C. under nitrogen atmosphere. The resulting mixture was stirred for 2 h at room temperature under nitrogen atmosphere. The reaction was monitored by LCMS. Desired product could be detected by LCMS. The resulting mixture was concentrated under vacuum. The residue was purified by silica gel column chromatography, eluted with PE/EA (10:1) to afford 1,5-di-tert-butyl 2-(3-fluoropyridin-4-yl)-4-oxo-7-(2-oxoethyl)-6H,7H-pyrrolo[3,2-c]pyridine-1,5-dicarboxylate (235 mg, 37.29%) as a yellow solid.

LC-MS: M+H found: 474.0.

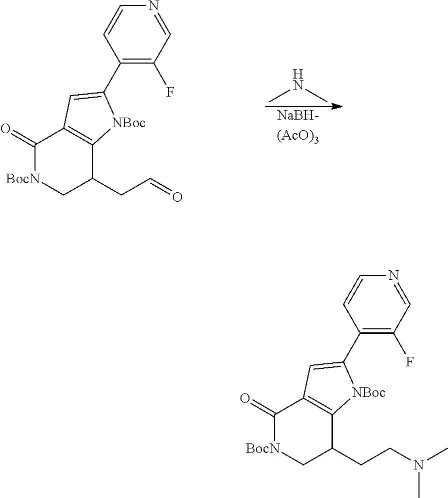

A solution of 1,5-di-tert-butyl 2-(3-fluoropyridin-4-yl)-4-oxo-7-(2-oxoethyl)-6H,7H-pyrrolo[3,2-c]pyridine-1,5-dicarboxylate (170 mg, 0.36 mmol, 1.00 equiv) in DCM (4 mL) was treated with diacetyl peroxide; sodioboranyl acetate (91 mg, 0.43 mmol, 1.20 equiv) for 30 min at room temperature under nitrogen atmosphere followed by the addition of dimethylamine (19 mg, 0.43 mmol, 1.20 equiv) dropwise at room temperature. The resulting mixture was stirred for 2 h at room temperature under nitrogen atmosphere. The reaction was monitored by LCMS. Desired product could be detected by LCMS. The resulting mixture was diluted with water (10 mL). The aqueous layer was extracted with CH₂Cl₂ (2×10 mL). The resulting mixture was concentrated under reduced pressure. The crude product was used in the next step directly without further purification.

LC-MS: M+H found: 503.0.

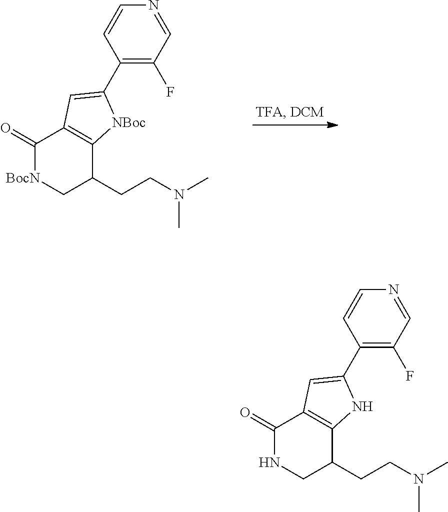 [SMILES: CN(C)CCC1CN(C(=O)OC(C)(C)C)C(=O)c2cc(-c3ccncc3F)n(C(=O)OC(C)(C)C)c21 ; CN(C)CCC1CNC(=O)c2cc(-c3ccncc3F)[nH]c21]

To a stirred mixture of 1,5-di-tert-butyl 2-(3-fluoropyridin-4-yl)-7-[2-(morpholin-4-yl)ethyl]-4-oxo-6H,7H-pyrrolo[3,2-c]pyridine-1,5-dicarboxylate (170 mg, 0.34 mmol, 1.00 equiv) in DCM (6 mL) was added TFA (2 mL) at room temperature under nitrogen atmosphere. The resulting mixture was stirred for 1 h and concentrated under reduced pressure. The residue was purified by reverse flash chromatography with the following conditions: column, C18 silica gel; mobile phase, ACN in water, 10% to 50% gradient in 10 min; detector, UV 254 nm to afford 7-[2-(dimethylamino)ethyl]-2-(3-fluoropyridin-4-yl)-1H,5H,6H,7H-pyrrolo[3,2-c]pyridin-4-one (100 mg, 97.78%) as a yellow solid.

LC-MS: (M+H)⁺ found: 303.

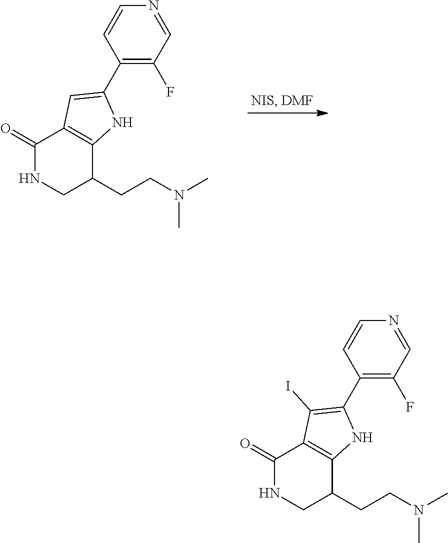 [SMILES: CN(C)CCC1CNC(=O)c2cc(-c3ccncc3F)[nH]c21 ; CN(C)CCC1CNC(=O)c2c1[nH]c(-c1ccncc1F)c2I]

A solution of 7-[2-(dimethylamino)ethyl]-2-(3-fluoropyridin-4-yl)-1H,5H,6H,7H-pyrrolo[3,2-c]pyridin-4-one (130 mg, 0.430 mmol, 1.00 equiv) and NIS (96.73 mg, 0.430 mmol, 1 equiv) in DMF (4 mL) was stirred for 2 h at room temperature under nitrogen atmosphere. The reaction was monitored by LCMS. Desired product could be detected by LCMS. The residue was purified by reverse flash chromatography with the following conditions: column, C18 silica gel; mobile phase, ACN in water, 10% to 50% gradient in 10 min; detector, UV 254 nm to afford 7-[2-(dimethylamino)ethyl]-2-(3-fluoropyridin-4-yl)-3-iodo-1H,5H,6H,7H-pyrrolo[3,2-c]pyridin-4-one (130 mg, 70.60%) as a yellow solid.

LC-MS: M+H found: 429.0.

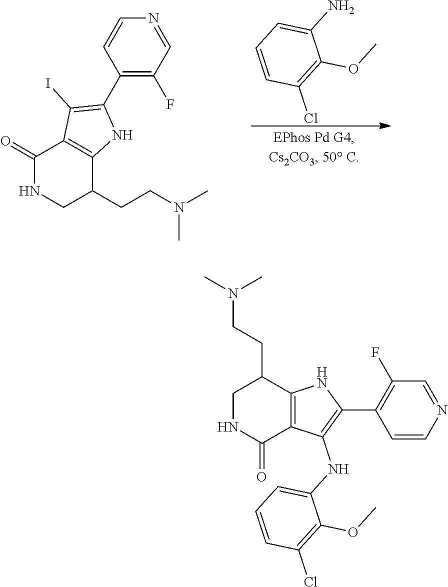

To a stirred mixture of 7-[2-(dimethylamino)ethyl]-2-(3-fluoropyridin-4-yl)-3-iodo-1H,5H,6H,7H-pyrrolo[3,2-c]pyridin-4-one (110 mg, 0.26 mmol, 1.00 equiv) and 3-chloro-2-methoxyaniline (40 mg, 0.26 mmol, 1.00 equiv) in DMF (2.5 mL) were added EPhos Pd G4 (24 mg, 0.026 mmol, 0.1 equiv) and Cs₂CO₃ (167 mg, 0.51 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, C18 silica gel; mobile phase, ACN in water, 10% to 50% gradient in 10 min; detector, UV 254 nm to afford 3-[(3-chloro-2-methoxyphenyl)amino]-7-[2-(dimethylamino)ethyl]-2-(3-fluoropyridin-4-yl)-1H,5H,6H,7H-pyrrolo[3,2-c]pyridin-4-one (70 mg, 59.51%) as a yellow solid.

LC-MS: M+H found: 458.0.

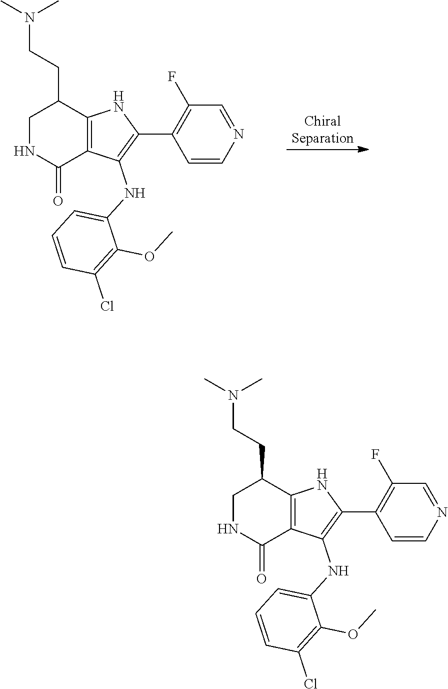 [SMILES: COc1c(Cl)cccc1Nc1c(-c2ccncc2F)[nH]c2c1C(=O)NCC2CCN(C)C ; COc1c(Cl)cccc1Nc1c(-c2ccncc2F)[nH]c2c1C(=O)NC[C@H]2CCN(C)C]

The crude product (80 mg) was purified by Prep-CHIRAL-HPLC with the following conditions (Column: CHIRALPAK IG-3, 4.6*50 mm, 3 um: Mobile Phase A: Hex (0.1% DEA): EtOH=70: 30; Flow rate: 1 mL/min; Gradient: 0% B to 0% B; Injection Volume: 5 ul mL) to afford (7R)-3-[(3-chloro-2-methoxyphenyl)amino]-7-[2-(dimethylamino)ethyl]-2-(3-fluoropyridin-4-yl)-1H,5H,6H,7H-pyrrolo[3,2-c]pyridin-4-one (18.5 mg, 22.22%) as a white solid.

¹H NMR (400 MHz, DMSO-d6) δ 12.10 (s, 1H), 8.59 (t, J=1.9 Hz, 1H), 8.33 (d, J=2.7 Hz, 1H), 7.72 (m, 1H), 7.38 (s, 1H), 7.13 (t, J=2.7 Hz, 1H), 6.80-6.63 (m, 2H), 6.16 (m, 1H), 3.89 (s, 3H), 3.48 (m, 1H), 3.20 (m, 1H), 3.09-2.96 (m, 1H), 2.48-2.38 (m, 1H), 2.32 (m, 1H), 2.23 (s, 6H), 1.97 (m, 1H). 1.73-1.55 (m, 1H).

LCMS found 458.

### Example 196. (7S)-3-[(3-chloro-2-methoxyphenyl)amino]-2-(3-fluoropyridin-4-yl)-7-[2-(morpholin-4-yl)ethyl]-1H,5H,6H,7H-pyrrolo[3,2-c]pyridin-4-one (compound 485)

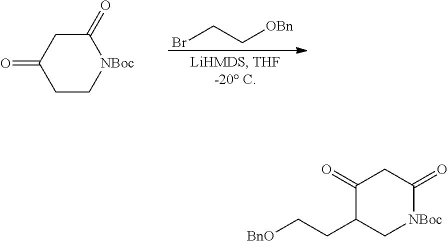 [SMILES: CC(C)(C)OC(=O)N1CCC(=O)CC1=O ; BrCCOCc1ccccc1 ; CC(C)(C)OC(=O)N1CC(CCOCc2ccccc2)C(=O)CC1=O]

To a stirred mixture of tert-butyl 2,4-dioxopiperidine-1-carboxylate (20 g, 93.794 mmol, 1.00 equiv) and [(2-bromoethoxy)methyl]benzene (80.70 g, 375.192 mmol, 4.00 equiv) in THF (600 mL) was added LiHMDS (235.00 mL, 235.000 mmol, 2.51 equiv) dropwise at −20 degrees C. under argon atmosphere. The resulting mixture was stirred for 1 h at −20 degrees C. under argon atmosphere. The reaction was quenched with Water at −20 degrees C. The mixture was acidified to pH=5 with 5% KHSO4 solution. The resulting mixture was extracted with CH2Cl2. The combined organic layers were washed with brine, dried over anhydrous Na2SO4. After filtration, the filtrate was concentrated under reduced pressure. The residue was purified by silica gel column chromatography, eluted with PE/EtOAc (2:1) to afford tert-butyl 5-[2-(benzyloxy)ethyl]-2,4-dioxopiperidine-1-carboxylate (17 g, 52.17%) as a green oil.

LC-MS: M+H found: 348.

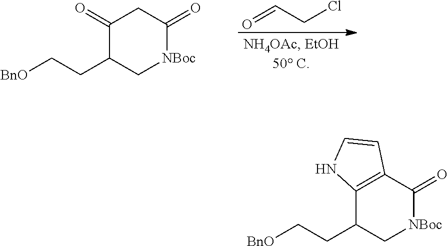

Into a round-bottom flask were added tert-butyl 5-[2-(benzyloxy)ethyl]-2,4-dioxopiperidine-1-carboxylate (10 g, 28.78 mmol, 1.00 equiv), Ammonia, 7.0 M Solution In Methanol, SpcSeal (10 mL), NH₄OAc (11.09 g, 143.92 mmol, 5.0 equiv), EtOH (100 mL) and chloroacetaldehyde (2.71 g, 34.54 mmol, 1.2 equiv) at room temperature. The resulting mixture was stirred for 1 h at 50 degrees C. under nitrogen atmosphere. The solvents were concentrated under reduced pressure. The reaction was quenched with Water at room temperature. The resulting mixture was extracted with EtOAc (3×200 mL). The combined organic layers were washed with brine (1×300 mL), dried over anhydrous Na2SO4. After filtration, the filtrate was concentrated under reduced pressure. The residue was purified by silica gel column chromatography, eluted with PE/EA (2:1) to afford tert-butyl 7-[2-(benzyloxy)ethyl]-4-oxo-1H,6H,7H-pyrrolo[3,2-c]pyridine-5-carboxylate (1.45 g, 13.60%) as a white solid.

LC-MS: M+H found: 371.

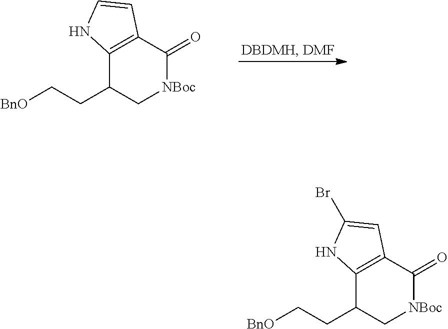 [SMILES: CC(C)(C)OC(=O)N1CC(CCOCc2ccccc2)c2[nH]ccc2C1=O ; CC(C)(C)OC(=O)N1CC(CCOCc2ccccc2)c2[nH]c(Br)cc2C1=O]

To a stirred solution of tert-butyl 7-[2-(benzyloxy)ethyl]-4-oxo-1H,6H,7H-pyrrolo[3,2-c]pyridine-5-carboxylate (1.05 g, 2.83 mmol, 1.00 equiv) in DMF (10 mL) was added dbdmh (0.39 g, 1.36 mmol, 0.48 equiv) in portions at −50 degrees C. under nitrogen atmosphere. The reaction was monitored by LCMS. Desired product could be detected by LCMS. The resulting mixture was diluted with water (10 mL). The resulting mixture was extracted with EtOAc (3×20 mL). The combined organic layers were washed with brine (3×10 mL), dried over anhydrous Na2SO4. After filtration, the filtrate was concentrated under reduced pressure. The residue was purified by silica gel column chromatography, eluted with PE/EA (5:1) to afford tert-butyl 7-[2-(benzyloxy)ethyl]-2-bromo-4-oxo-1H,6H,7H-pyrrolo[3,2-c]pyridine-5-carboxylate (1200 mg, 83.86%) as a white oil.

LC-MS: M+H found: 449.0.

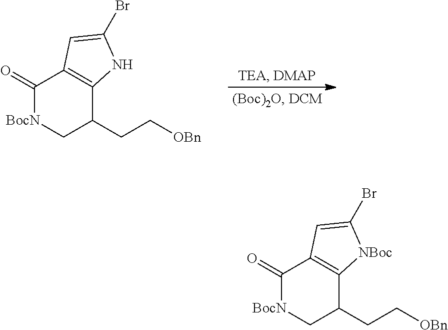 [SMILES: CC(C)(C)OC(=O)N1CC(CCOCc2ccccc2)c2[nH]c(Br)cc2C1=O ; CC(C)(C)OC(=O)N1CC(CCOCc2ccccc2)c2c(cc(Br)n2C(=O)OC(C)(C)C)C1=O]

A solution of tert-butyl 7-[2-(benzyloxy)ethyl]-2-bromo-4-oxo-1H,6H,7H-pyrrolo[3,2-c]pyridine-5-carboxylate (500 mg, 1.11 mmol, 1.00 equiv) and (Boc)₂O (267 mg, 1.224 mmol, 1.1 equiv) and TEA (135 mg, 1.34 mmol, 1.2 equiv) in DCM (5.00 mL) was stirred for 2 h at room temperature under nitrogen atmosphere. The reaction was monitored by LCMS. Desired product could be detected by LCMS. The resulting mixture was concentrated under reduced pressure. The residue was purified by Prep-TLC (PE/EA 5:1) to afford 1,5-di-tert-butyl 7-[2-(benzyloxy)ethyl]-2-bromo-4-oxo-6H,7H-pyrrolo[3,2-c]pyridine-1,5-dicarboxylate (590 mg, 92.64%) as a white oil.

LC-MS: M+H found: 549.0.

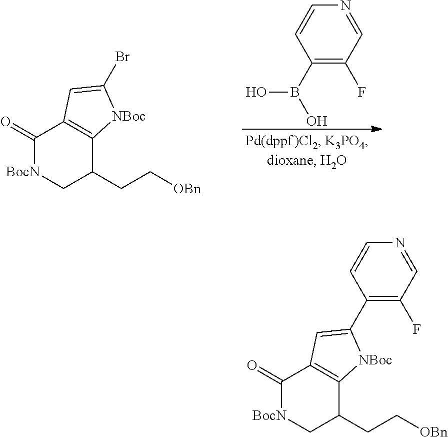 [SMILES: CC(C)(C)OC(=O)N1CC(CCOCc2ccccc2)c2c(cc(Br)n2C(=O)OC(C)(C)C)C1=O ; OB(O)c1ccncc1F ; CC(C)(C)OC(=O)N1CC(CCOCc2ccccc2)c2c(cc(-c3ccncc3F)n2C(=O)OC(C)(C)C)C1=O]

A solution of 1,5-di-tert-butyl 7-[2-(benzyloxy)ethyl]-2-bromo-4-oxo-6H,7H-pyrrolo[3,2-c]pyridine-1,5-dicarboxylate (6.5 g, 11.83 mmol, 1.00 equiv) and 3-fluoropyridin-4-ylboronic acid (2.50 g, 17.74 mmol, 1.5 equiv) and Pd(dppf)Cl₂·CH₂Cl₂ (964 mg, 1.18 mmol, 0.1 equiv) and K₃PO₄ (7.53 g, 35.49 mmol, 3 equiv) in dioxane (100 mL) and H2O (20 mL) was stirred for 1 h at 50 degrees C. under nitrogen atmosphere. The reaction was monitored by LCMS. Desired product could be detected by LCMS. The resulting mixture was concentrated under vacuum. The aqueous layer was extracted with EtOAc (4×50 mL). The residue was purified by silica gel column chromatography, eluted with PE/EA (10:1) to afford 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (5.6 g, 82.02%) as a yellow oil.

LC-MS: M+H found: 566.0.

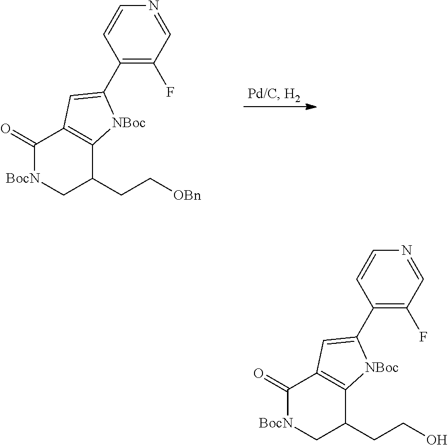 [SMILES: CC(C)(C)OC(=O)N1CC(CCOCc2ccccc2)c2c(cc(-c3ccncc3F)n2C(=O)OC(C)(C)C)C1=O ; CC(C)(C)OC(=O)N1CC(CCO)c2c(cc(-c3ccncc3F)n2C(=O)OC(C)(C)C)C1=O]

To a solution of 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (4 g, 7.072 mmol, 1.00 equiv) in 140 mL MeOH was added dry Pd/C (10%, 1.6 g) under nitrogen atmosphere in a 500 mL round-bottom flask. The mixture was hydrogenated at room temperature for 2 days under hydrogen atmosphere using a hydrogen balloon, filtered through a Celite pad and concentrated under reduced pressure. The reaction was monitored by LCMS. Desired product could be detected by LCMS. The resulting mixture was filtered, the filter cake was washed with MeOH (3×50 mL). The filtrate was concentrated under reduced pressure to afford 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (2.27 g, 57.38%) as a white oil.

LC-MS: M+H found: 476.0.

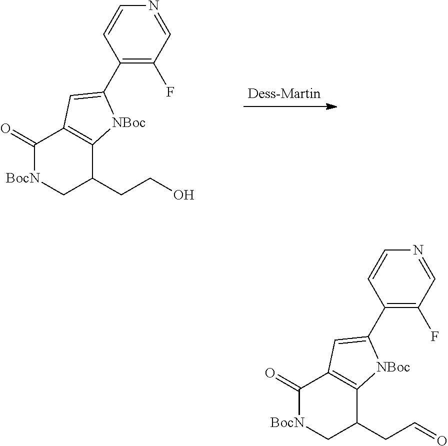 [SMILES: CC(C)(C)OC(=O)N1CC(CCO)c2c(cc(-c3ccncc3F)n2C(=O)OC(C)(C)C)C1=O ; CC(C)(C)OC(=O)N1CC(CC=O)c2c(cc(-c3ccncc3F)n2C(=O)OC(C)(C)C)C1=O]

To a stirred solution of 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (500 mg, 1.05 mmol, 1.00 equiv) in DCM (10 mL) were added Dess-Martin (535 mg, 1.26 mmol, 1.20 equiv) in portions at 0 degrees C. under nitrogen atmosphere. The resulting mixture was stirred for 2 h at room temperature under nitrogen atmosphere. The reaction was monitored by LCMS. Desired product could be detected by LCMS. The resulting mixture was concentrated under vacuum. The residue was purified by silica gel column chromatography, eluted with PE/EA (10:1) to afford 1,5-di-tert-butyl 2-(3-fluoropyridin-4-yl)-4-oxo-7-(2-oxoethyl)-6H,7H-pyrrolo[3,2-c]pyridine-1,5-dicarboxylate (235 mg, 37.29%) as a yellow solid.

LC-MS: M+H found: 474.0.

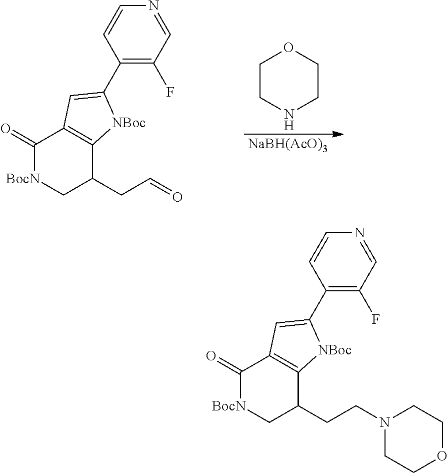

A solution of 1,5-di-tert-butyl 2-(3-fluoropyridin-4-yl)-4-oxo-7-(2-oxoethyl)-6H,7H-pyrrolo[3,2-c]pyridine-1,5-dicarboxylate (280 mg, 0.59 mmol, 1.00 equiv) in DCM (7.00 mL) was treated with diacetyl peroxide; sodioboranyl acetate (150 mg, 0.71 mmol, 1.20 equiv) for 30 min at room temperature under nitrogen atmosphere followed by the addition of morpholine (62 mg, 0.71 mmol, 1.20 equiv) dropwise at room temperature. The resulting mixture was stirred for 2 h at room temperature under nitrogen atmosphere. The reaction was monitored by LCMS. Desired product could be detected by LCMS. The reaction was quenched with Water at 0 degrees C. The resulting mixture was extracted with CH₂Cl₂ (3×20 mL). The combined organic layers were washed with brine (2×5 mL), dried over anhydrous Na₂SO₄. After filtration, the filtrate was concentrated under reduced pressure. The crude product was used in the next step directly without further purification.

LC-MS: M+H found: 545.0.

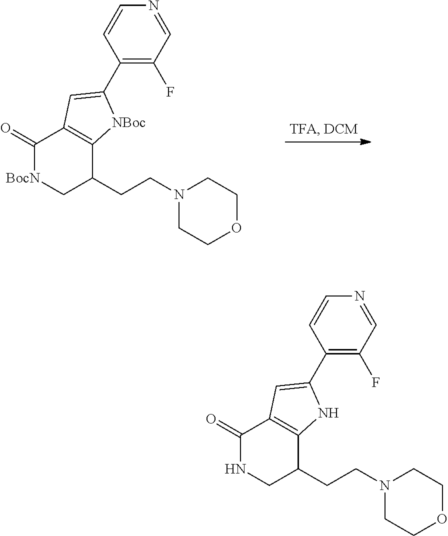 [SMILES: CC(C)(C)OC(=O)N1CC(CCN2CCOCC2)c2c(cc(-c3ccncc3F)n2C(=O)OC(C)(C)C)C1=O ; O=C1NCC(CCN2CCOCC2)c2[nH]c(-c3ccncc3F)cc21]

To a stirred mixture of 1,5-di-tert-butyl 2-(3-fluoropyridin-4-yl)-7-[2-(morpholin-4-yl)ethyl]-4-oxo-6H,7H-pyrrolo[3,2-c]pyridine-1,5-dicarboxylate (280 mg, 0.514 mmol, 1.00 equiv) in DCM (7.5 mL) was added TFA (2.5 mL) at room temperature under nitrogen atmosphere. The resulting mixture was stirred for 1 h and concentrated under reduced pressure. The residue was purified by reverse flash chromatography with the following conditions: column, C18 silica gel; mobile phase, ACN in water, 10% to 50% gradient in 10 min; detector, UV 254 nm to afford 2-(3-fluoropyridin-4-yl)-7-[2-(morpholin-4-yl)ethyl]-1H,5H,6H,7H-pyrrolo[3,2-c]pyridin-4-one (140 mg, 77.49%) as a light yellow solid.

LC-MS: (M+H)⁺ found: 345.

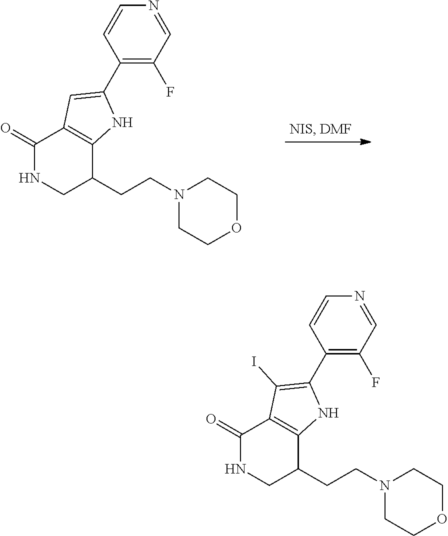 [SMILES: O=C1NCC(CCN2CCOCC2)c2[nH]c(-c3ccncc3F)cc21 ; O=C1NCC(CCN2CCOCC2)c2[nH]c(-c3ccncc3F)c(I)c21]

To a stirred solution of 2-(3-fluoropyridin-4-yl)-7-[2-(morpholin-4-yl)ethyl]-1H,5H,6H,7H-pyrrolo[3,2-c]pyridin-4-one (130 mg, 0.38 mmol, 1.00 equiv) in DMF (7 mL) was added NIS (85 mg, 0.38 mmol, 1 equiv) in portions at room temperature under nitrogen atmosphere. The resulting mixture was stirred for 2 h at room temperature under nitrogen atmosphere. The reaction was monitored by LCMS. The residue was purified by reverse flash chromatography with the following conditions; column, C18 silica gel; mobile phase, ACN in water, 10% to 50% gradient in 10 min; detector, UV 254 nm to afford 2-(3-fluoropyridin-4-yl)-3-iodo-7-[2-(morpholin-4-yl)ethyl]-1H,5H,6H,7H-pyrrolo[3,2-c]pyridin-4-one (167 mg, 94.07%) as a yellow solid. Desired product could be detected by LCMS.

LC-MS: M+H found: 471.0.

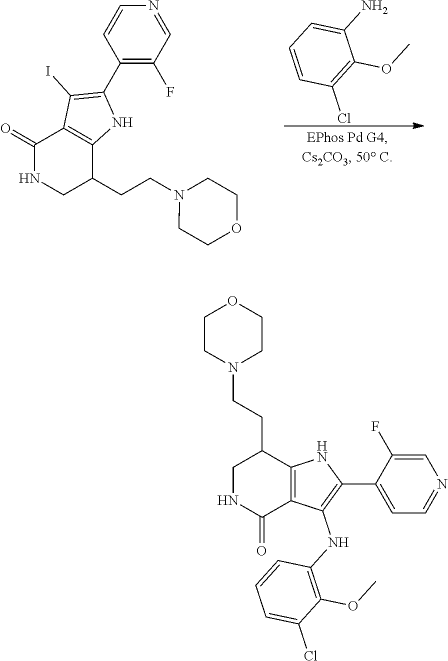

To a stirred mixture of 2-(3-fluoropyridin-4-yl)-3-iodo-7-[2-(morpholin-4-yl)ethyl]-1H,5H,6H,7H-pyrrolo[3,2-c]pyridin-4-one (80 mg, 0.17 mmol, 1.00 equiv) and 3-chloro-2-methoxyaniline (26.81 mg, 0.17 mmol, 1 equiv) in DMF (2 mL) were added EPhos Pd G4 (16 mg, 0.017 mmol, 0.1 equiv) and Cs₂CO₃ (110 mg, 0.34 mmol, 2 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, C18 silica gel; mobile phase, ACN in water, 10% to 50% gradient in 10 min; detector, UV 254 nm to afford 3-[(3-chloro-2-methoxyphenyl)amino]-2-(3-fluoropyridin-4-yl)-7-[2-(morpholin-4-yl)ethyl]-1H,5H,6H,7H-pyrrolo[3,2-c]pyridin-4-one (42 mg, 49.38%) as a yellow solid.

LC-MS: M+H found: 500.0.

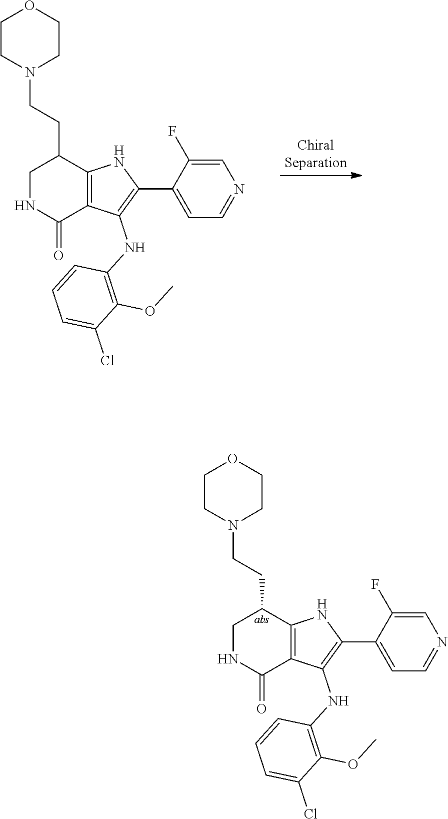 [SMILES: COc1c(Cl)cccc1Nc1c(-c2ccncc2F)[nH]c2c1C(=O)NCC2CCN1CCOCC1 ; COc1c(Cl)cccc1Nc1c(-c2ccncc2F)[nH]c2c1C(=O)NC[C@@H]2CCN1CCOCC1]

The product (42 mg) was purified by Prep-Chiral-HPLC with the following conditions (Column: CHIRALPAK IG-3, 4.6*50 mm, 3 m; Mobile Phase A: (Hex:DCM=3:1) (0.1% DEA): EtOH=90: 10; Flow rate: 1 mL/min; Gradient: 0% B to 0% B; Injection Volume: 5 ul mL) to afford (7S)-3-[(3-chloro-2-methoxyphenyl)amino]-2-(3-fluoropyridin-4-yl)-7-[2-(morpholin-4-yl)ethyl]-1H,5H,6H,7H-pyrrolo[3,2-c]pyridin-4-one (16.2 mg, 37.34%) as a yellow solid.

LC-MS: (M+H)⁺ found 500.

¹H NMR (400 MHz, DMSO-d₆) δ 11.65 (s, 1H), 8.62 (t, J=1.9 Hz, 1H), 8.33 (d, J=2.7 Hz, 1H), 7.77 (m, 1H), 7.38 (s, 1H), 7.14 (t, J=2.6 Hz, 1H), 6.81-6.65 (m, 2H), 6.15 (m, 1H), 3.89 (s, 3H), 3.59 (t, J=4.8 Hz, 4H), 3.51 (m, 1H), 3.22 (m, 1H), 3.03 (dq, J=10.4, 5.4 Hz, 1H), 2.40 (m, 6H), 2.13-2.00 (m, 1H), 1.67 (m, 1H).

### Example 197. (7R)-3-[(3-chloro-2-methoxyphenyl)amino]-2-(3-fluoropyridin-4-yl)-7-[2-(morpholin-4-yl)ethyl]-1H,5H,6H,7H-pyrrolo[3,2-c]pyridin-4-one (compound 484)

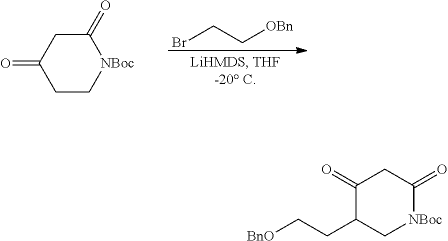 [SMILES: CC(C)(C)OC(=O)N1CCC(=O)CC1=O ; BrCCOCc1ccccc1 ; CC(C)(C)OC(=O)N1CC(CCOCc2ccccc2)C(=O)CC1=O]

To a stirred mixture of tert-butyl 2,4-dioxopiperidine-1-carboxylate (20 g, 93.794 mmol, 1.00 equiv) and [(2-bromoethoxy)methyl]benzene (80.70 g, 375.192 mmol, 4.00 equiv) in THF (600 mL) was added LiHMDS (235.00 mL, 235.000 mmol, 2.51 equiv) dropwise at −20 degrees C. under argon atmosphere. The resulting mixture was stirred for 1 h at −20 degrees C. under argon atmosphere. The reaction was quenched with Water at −20 degrees C. The mixture was acidified to pH=5 with 5% KHSO4 solution. The resulting mixture was extracted with CH2Cl2. The combined organic layers were washed with brine, dried over anhydrous Na2SO4. After filtration, the filtrate was concentrated under reduced pressure. The residue was purified by silica gel column chromatography, eluted with PE/EtOAc (2:1) to afford tert-butyl 5-[2-(benzyloxy)ethyl]-2,4-dioxopiperidine-1-carboxylate (17 g, 52.17%) as a green oil.

LC-MS: M+H found: 348.

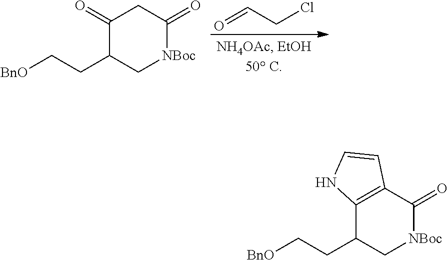

Into a round-bottom flask were added tert-butyl 5-[2-(benzyloxy)ethyl]-2,4-dioxopiperidine-1-carboxylate (10 g, 28.78 mmol, 1.00 equiv), Ammonia, 7.0 M Solution In Methanol, SpcSeal (10 mL), NH₄OAc (11.09 g, 143.92 mmol, 5.0 equiv), EtOH (100 mL) and chloroacetaldehyde (2.71 g, 34.54 mmol, 1.2 equiv) at room temperature. The resulting mixture was stirred for 1 h at 50 degrees C. under nitrogen atmosphere. The solvents were concentrated under reduced pressure. The reaction was quenched with Water at room temperature. The resulting mixture was extracted with EtOAc (3×200 mL). The combined organic layers were washed with brine (1×300 mL), dried over anhydrous Na2SO4. After filtration, the filtrate was concentrated under reduced pressure. The residue was purified by silica gel column chromatography, eluted with PE/EA (2:1) to afford tert-butyl 7-[2-(benzyloxy)ethyl]-4-oxo-1H,6H,7H-pyrrolo[3,2-c]pyridine-5-carboxylate (1.45 g, 13.60%) as a white solid.

LC-MS: M+H found: 371.

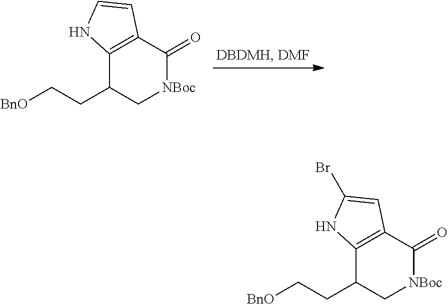 [SMILES: CC(C)(C)OC(=O)N1CC(CCOCc2ccccc2)c2[nH]ccc2C1=O ; CC(C)(C)OC(=O)N1CC(CCOCc2ccccc2)c2[nH]c(Br)cc2C1=O]

To a stirred solution of tert-butyl 7-[2-(benzyloxy)ethyl]-4-oxo-1H,6H,7H-pyrrolo[3,2-c]pyridine-5-carboxylate (1.05 g, 2.83 mmol, 1.00 equiv) in DMF (10 mL) was added dbdmh (0.39 g, 1.36 mmol, 0.48 equiv) in portions at −50 degrees C. under nitrogen atmosphere. The reaction was monitored by LCMS. Desired product could be detected by LCMS. The resulting mixture was diluted with water (10 mL). The resulting mixture was extracted with EtOAc (3×20 mL). The combined organic layers were washed with brine (3×10 mL), dried over anhydrous Na2SO4. After filtration, the filtrate was concentrated under reduced pressure. The residue was purified by silica gel column chromatography, eluted with PE/EA (5:1) to afford tert-butyl 7-[2-(benzyloxy)ethyl]-2-bromo-4-oxo-1H,6H,7H-pyrrolo[3,2-c]pyridine-5-carboxylate (1200 mg, 83.86%) as a white oil.

LC-MS: M+H found: 449.0.

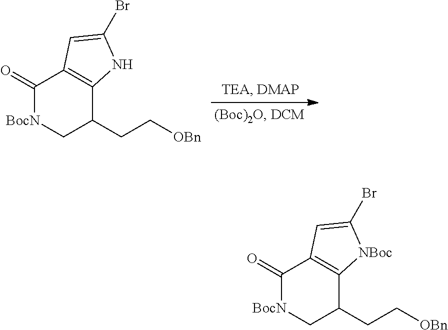 [SMILES: CC(C)(C)OC(=O)N1CC(CCOCc2ccccc2)c2[nH]c(Br)cc2C1=O ; CC(C)(C)OC(=O)N1CC(CCOCc2ccccc2)c2c(cc(Br)n2C(=O)OC(C)(C)C)C1=O]

A solution of tert-butyl 7-[2-(benzyloxy)ethyl]-2-bromo-4-oxo-1H,6H,7H-pyrrolo[3,2-c]pyridine-5-carboxylate (500 mg, 1.11 mmol, 1.00 equiv) and (Boc)₂O (267 mg, 1.224 mmol, 1.1 equiv) and TEA (135 mg, 1.34 mmol, 1.2 equiv) in DCM (5.00 mL) was stirred for 2 h at room temperature under nitrogen atmosphere. The reaction was monitored by LCMS. Desired product could be detected by LCMS. The resulting mixture was concentrated under reduced pressure. The residue was purified by Prep-TLC (PE/EA 5:1) to afford 1,5-di-tert-butyl 7-[2-(benzyloxy)ethyl]-2-bromo-4-oxo-6H,7H-pyrrolo[3,2-c]pyridine-1,5-dicarboxylate (590 mg, 92.64%) as a white oil.

LC-MS: M+H found: 549.0.

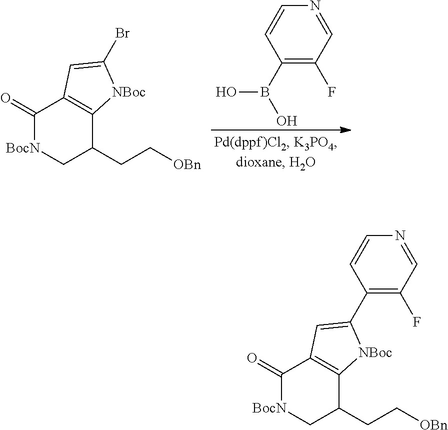 [SMILES: CC(C)(C)OC(=O)N1CC(CCOCc2ccccc2)c2c(cc(Br)n2C(=O)OC(C)(C)C)C1=O ; OB(O)c1ccncc1F ; CC(C)(C)OC(=O)N1CC(CCOCc2ccccc2)c2c(cc(-c3ccncc3F)n2C(=O)OC(C)(C)C)C1=O]

A solution of 1,5-di-tert-butyl 7-[2-(benzyloxy)ethyl]-2-bromo-4-oxo-6H,7H-pyrrolo[3,2-c]pyridine-1,5-dicarboxylate (6.5 g, 11.83 mmol, 1.00 equiv) and 3-fluoropyridin-4-ylboronic acid (2.50 g, 17.74 mmol, 1.5 equiv) and Pd(dppf)Cl₂·CH₂Cl₂ (964 mg, 1.18 mmol, 0.1 equiv) and K₃PO₄ (7.53 g, 35.49 mmol, 3 equiv) in dioxane (100 mL) and H₂O (20 mL) was stirred for 1 h at 50 degrees C. under nitrogen atmosphere. The reaction was monitored by LCMS. Desired product could be detected by LCMS. The resulting mixture was concentrated under vacuum. The aqueous layer was extracted with EtOAc (4×50 mL). The residue was purified by silica gel column chromatography, eluted with PE/EA (10:1) to afford 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (5.6 g, 82.02%) as a yellow oil.

LC-MS: M+H found: 566.0.

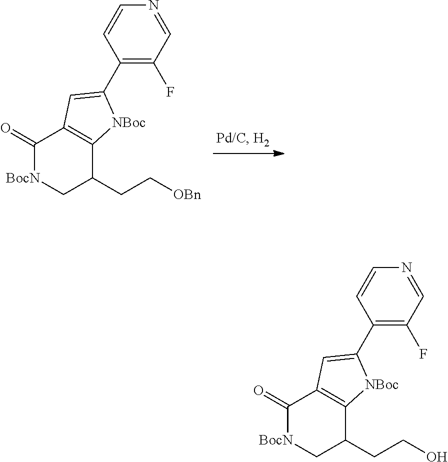 [SMILES: CC(C)(C)OC(=O)N1CC(CCOCc2ccccc2)c2c(cc(-c3ccncc3F)n2C(=O)OC(C)(C)C)C1=O ; CC(C)(C)OC(=O)N1CC(CCO)c2c(cc(-c3ccncc3F)n2C(=O)OC(C)(C)C)C1=O]

To a solution of 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (4 g, 7.072 mmol, 1.00 equiv) in 140 mL MeOH was added dry Pd/C (10%, 1.6 g) under nitrogen atmosphere in a 500 mL round-bottom flask. The mixture was hydrogenated at room temperature for 2 days under hydrogen atmosphere using a hydrogen balloon, filtered through a Celite pad and concentrated under reduced pressure. The reaction was monitored by LCMS. Desired product could be detected by LCMS. The resulting mixture was filtered, the filter cake was washed with MeOH (3×50 mL). The filtrate was concentrated under reduced pressure to afford 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (2.27 g, 57.38%) as a white oil.

LC-MS: M+H found: 476.0.

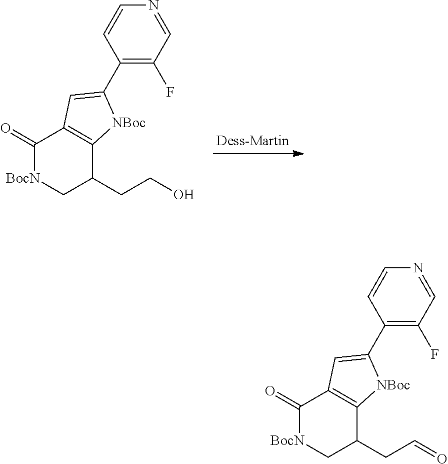 [SMILES: CC(C)(C)OC(=O)N1CC(CCO)c2c(cc(-c3ccncc3F)n2C(=O)OC(C)(C)C)C1=O ; CC(C)(C)OC(=O)N1CC(CC=O)c2c(cc(-c3ccncc3F)n2C(=O)OC(C)(C)C)C1=O]

To a stirred solution of 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (500 mg, 1.05 mmol, 1.00 equiv) in DCM (10 mL) were added Dess-Martin (535 mg, 1.26 mmol, 1.20 equiv) in portions at 0 degrees C. under nitrogen atmosphere. The resulting mixture was stirred for 2 h at room temperature under nitrogen atmosphere. The reaction was monitored by LCMS. Desired product could be detected by LCMS. The resulting mixture was concentrated under vacuum. The residue was purified by silica gel column chromatography, eluted with PE/EA (10:1) to afford 1,5-di-tert-butyl 2-(3-fluoropyridin-4-yl)-4-oxo-7-(2-oxoethyl)-6H,7H-pyrrolo[3,2-c]pyridine-1,5-dicarboxylate (235 mg, 37.29%) as a yellow solid.

LC-MS: M+H found: 474.0.

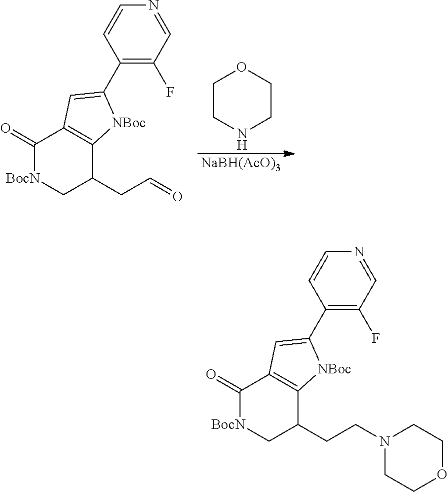

A solution of 1,5-di-tert-butyl 2-(3-fluoropyridin-4-yl)-4-oxo-7-(2-oxoethyl)-6H,7H-pyrrolo[3,2-c]pyridine-1,5-dicarboxylate (280 mg, 0.59 mmol, 1.00 equiv) in DCM (7.00 mL) was treated with diacetyl peroxide; sodioboranyl acetate (150 mg, 0.71 mmol, 1.20 equiv) for 30 min at room temperature under nitrogen atmosphere followed by the addition of morpholine (62 mg, 0.71 mmol, 1.20 equiv) dropwise at room temperature. The resulting mixture was stirred for 2 h at room temperature under nitrogen atmosphere. The reaction was monitored by LCMS. Desired product could be detected by LCMS. The reaction was quenched with Water at 0 degrees C. The resulting mixture was extracted with CH₂Cl₂ (3×20 mL). The combined organic layers were washed with brine (2×5 mL), dried over anhydrous Na₂SO₄. After filtration, the filtrate was concentrated under reduced pressure. The crude product was used in the next step directly without further purification.

LC-MS: M+H found: 545.0.

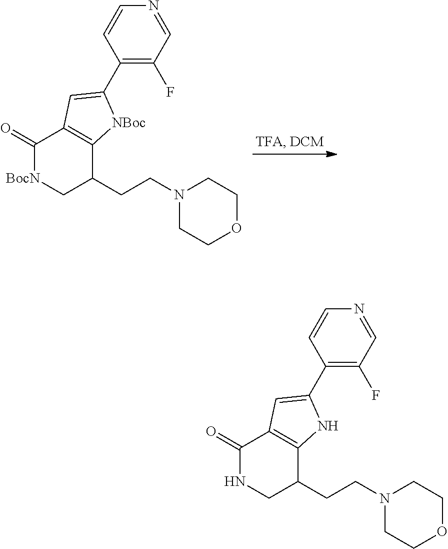 [SMILES: CC(C)(C)OC(=O)N1CC(CCN2CCOCC2)c2c(cc(-c3ccncc3F)n2C(=O)OC(C)(C)C)C1=O ; O=C1NCC(CCN2CCOCC2)c2[nH]c(-c3ccncc3F)cc21]

To a stirred mixture of 1,5-di-tert-butyl 2-(3-fluoropyridin-4-yl)-7-[2-(morpholin-4-yl)ethyl]-4-oxo-6H,7H-pyrrolo[3,2-c]pyridine-1,5-dicarboxylate (280 mg, 0.514 mmol, 1.00 equiv) in DCM (7.5 mL) was added TFA (2.5 mL) at room temperature under nitrogen atmosphere. The resulting mixture was stirred for 1 h and concentrated under reduced pressure. The residue was purified by reverse flash chromatography with the following conditions: column, C18 silica gel; mobile phase, ACN in water, 10% to 50% gradient in 10 min; detector, UV 254 nm to afford 2-(3-fluoropyridin-4-yl)-7-[2-(morpholin-4-yl)ethyl]-1H,5H,6H,7H-pyrrolo[3,2-c]pyridin-4-one (140 mg, 77.49%) as a light yellow solid.

LC-MS: (M+H)⁺ found: 345.

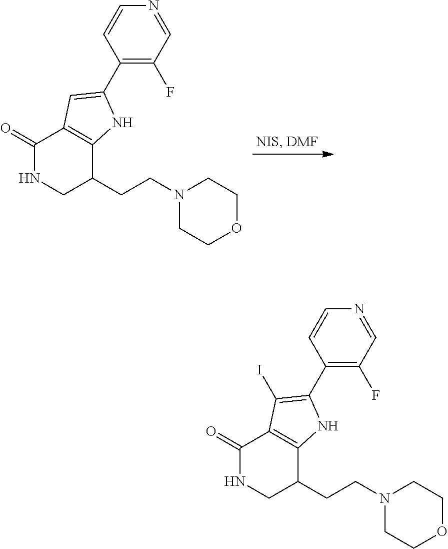 [SMILES: O=C1NCC(CCN2CCOCC2)c2[nH]c(-c3ccncc3F)cc21 ; O=C1NCC(CCN2CCOCC2)c2[nH]c(-c3ccncc3F)c(I)c21]

To a stirred solution of 2-(3-fluoropyridin-4-yl)-7-[2-(morpholin-4-yl)ethyl]-1H,5H,6H,7H-pyrrolo[3,2-c]pyridin-4-one (130 mg, 0.38 mmol, 1.00 equiv) in DMF (7 mL) was added NIS (85 mg, 0.38 mmol, 1 equiv) in portions at room temperature under nitrogen atmosphere. The resulting mixture was stirred for 2 h at room temperature under nitrogen atmosphere. The reaction was monitored by LCMS. The residue was purified by reverse flash chromatography with the following conditions: column, C18 silica gel; mobile phase, ACN in water, 10% to 50% gradient in 10 min; detector, UV 254 nm to afford 2-(3-fluoropyridin-4-yl)-3-iodo-7-[2-(morpholin-4-yl)ethyl]-1H,5H,6H,7H-pyrrolo[3,2-c]pyridin-4-one (167 mg, 94.07%) as a yellow solid. Desired product could be detected by LCMS.

LC-MS: M+H found: 471.0.

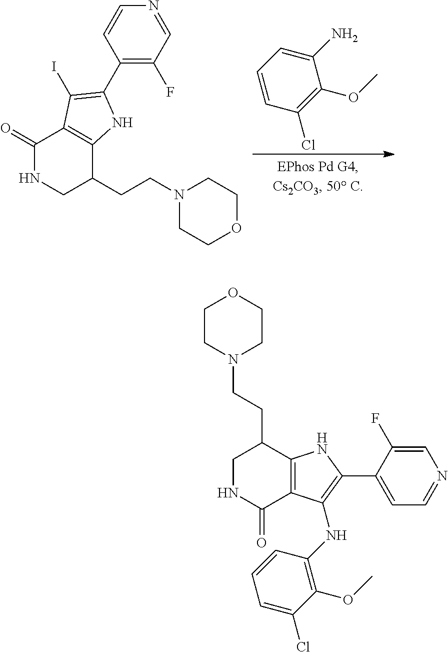

To a stirred mixture of 2-(3-fluoropyridin-4-yl)-3-iodo-7-[2-(morpholin-4-yl)ethyl]-1H,5H,6H,7H-pyrrolo[3,2-c]pyridin-4-one (80 mg, 0.17 mmol, 1.00 equiv) and 3-chloro-2-methoxyaniline (26.81 mg, 0.17 mmol, 1 equiv) in DMF (2 mL) were added EPhos Pd G4 (16 mg, 0.017 mmol, 0.1 equiv) and Cs₂CO₃ (110 mg, 0.34 mmol, 2 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, C18 silica gel; mobile phase, ACN in water, 10% to 50% gradient in 10 min; detector, UV 254 nm to afford 3-[(3-chloro-2-methoxyphenyl)amino]-2-(3-fluoropyridin-4-yl)-7-[2-(morpholin-4-yl)ethyl]-1H,5H,6H,7H-pyrrolo[3,2-c]pyridin-4-one (42 mg, 49.38%) as a yellow solid.

LC-MS: M+H found: 500.0.

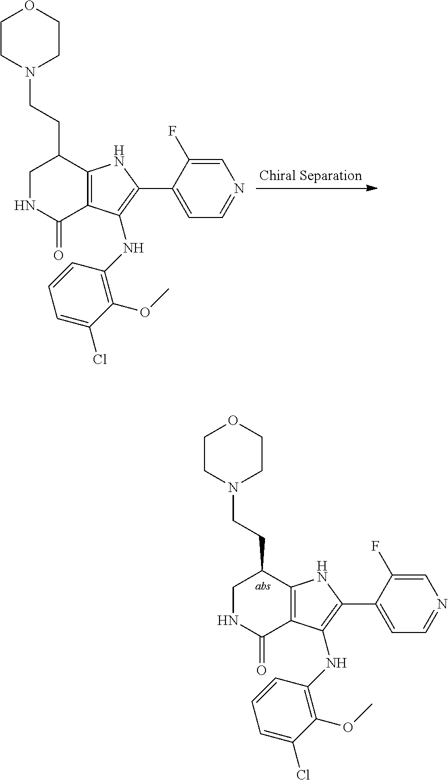 [SMILES: COc1c(Cl)cccc1Nc1c(-c2ccncc2F)[nH]c2c1C(=O)NCC2CCN1CCOCC1 ; COc1c(Cl)cccc1Nc1c(-c2ccncc2F)[nH]c2c1C(=O)NC[C@H]2CCN1CCOCC1]

The product (42 mg) was purified by Prep-Chiral-HPLC with the following conditions (Column: CHIRALPAK IG-3, 4.6*50 mm, 3 m; Mobile Phase A: (Hex:DCM=3:1) (0.1% DEA): EtOH=90: 10; Flow rate: 1 mL/min; Gradient: 0% B to 0% B; Injection Volume: 5 ul mL) to afford (7R)-3-[(3-chloro-2-methoxyphenyl)amino]-2-(3-fluoropyridin-4-yl)-7-[2-(morpholin-4-yl)ethyl]-1H,5H,6H,7H-pyrrolo[3,2-c]pyridin-4-one (13.6 mg, 32.19%) as a white solid.

LC-MS: (M+H)⁺ found: 500.

¹H NMR (400 MHz, DMSO-d₆) δ 11.64 (s, 1H), 8.62 (t, J=1.9 Hz, 1H), 8.33 (d, J=2.7 Hz, 1H), 7.77 (m, 1H), 7.38 (s, 1H), 7.14 (d, J=2.6 Hz, 1H), 6.77-6.64 (m, 2H), 6.15 (m, 1H), 3.89 (s, 3H), 3.59 (t, J=4.8 Hz, 4H), 3.51 m, 1H), 3.22 m, 1H), 3.03 (d, J=4.9 Hz, 1H), 2.48-2.26 (m, 6H), 2.14-1.99 (m, 1H), 1.67 (m, 1H).

### Example 198. (7S)-3-[(3-chloro-2-methoxyphenyl)amino]-7-[2-(dimethylamino)ethyl]-2-(3-fluoropyridin-4-yl)-1H,5H,6H,7H-pyrrolo[3,2-c]pyridin-4-one (compound 475)

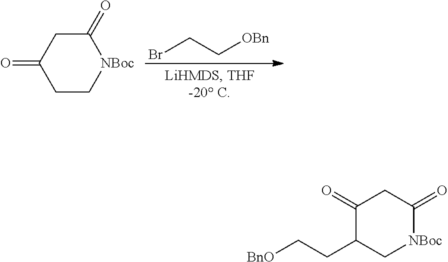 [SMILES: CC(C)(C)OC(=O)N1CCC(=O)CC1=O ; BrCCOCc1ccccc1 ; CC(C)(C)OC(=O)N1CC(CCOCc2ccccc2)C(=O)CC1=O]

To a stirred mixture of tert-butyl 2,4-dioxopiperidine-1-carboxylate (20 g, 93.794 mmol, 1.00 equiv) and [(2-bromoethoxy)methyl]benzene (80.70 g, 375.192 mmol, 4.00 equiv) in THF (600 mL) was added LiHMDS (235.00 mL, 235.000 mmol, 2.51 equiv) dropwise at −20 degrees C. under argon atmosphere. The resulting mixture was stirred for 1 h at −20 degrees C. under argon atmosphere. The reaction was quenched with Water at −20 degrees C. The mixture was acidified to pH=5 with 5% KHSO4 solution. The resulting mixture was extracted with CH2Cl2. The combined organic layers were washed with brine, dried over anhydrous Na2SO4. After filtration, the filtrate was concentrated under reduced pressure. The residue was purified by silica gel column chromatography, eluted with PE/EtOAc (2:1) to afford tert-butyl 5-[2-(benzyloxy)ethyl]-2,4-dioxopiperidine-1-carboxylate (17 g, 52.17%) as a green oil.

LC-MS: M+H found: 348.

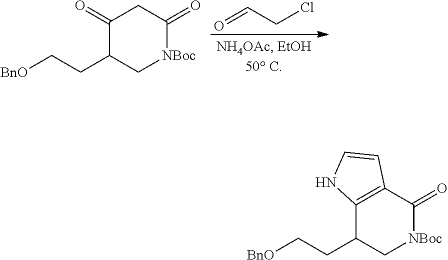

Into a round-bottom flask were added tert-butyl 5-[2-(benzyloxy)ethyl]-2,4-dioxopiperidine-1-carboxylate (10 g, 28.78 mmol, 1.00 equiv), Ammonia, 7.0 M Solution In Methanol, SpcSeal (10 mL), NH₄OAc (11.09 g, 143.92 mmol, 5.0 equiv), EtOH (100 mL) and chloroacetaldehyde (2.71 g, 34.54 mmol, 1.2 equiv) at room temperature. The resulting mixture was stirred for 1 h at 50 degrees C. under nitrogen atmosphere. The solvents were concentrated under reduced pressure. The reaction was quenched with Water at room temperature. The resulting mixture was extracted with EtOAc (3×200 mL). The combined organic layers were washed with brine (1×300 mL), dried over anhydrous Na₂SO₄. After filtration, the filtrate was concentrated under reduced pressure. The residue was purified by silica gel column chromatography, eluted with PE/EA (2:1) to afford tert-butyl 7-[2-(benzyloxy)ethyl]-4-oxo-1H,6H,7H-pyrrolo[3,2-c]pyridine-5-carboxylate (1.45 g, 13.60%) as a white solid.

LC-MS: M+H found: 371.

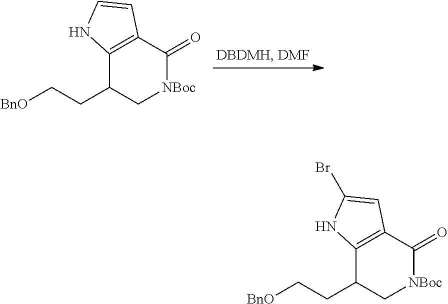 [SMILES: CC(C)(C)OC(=O)N1CC(CCOCc2ccccc2)c2[nH]ccc2C1=O ; CC(C)(C)OC(=O)N1CC(CCOCc2ccccc2)c2[nH]c(Br)cc2C1=O]

To a stirred solution of tert-butyl 7-[2-(benzyloxy)ethyl]-4-oxo-1H,6H,7H-pyrrolo[3,2-c]pyridine-5-carboxylate (1.05 g, 2.83 mmol, 1.00 equiv) in DMF (10 mL) was added dbdmh (0.39 g, 1.36 mmol, 0.48 equiv) in portions at −50 degrees C. under nitrogen atmosphere. The reaction was monitored by LCMS. Desired product could be detected by LCMS. The resulting mixture was diluted with water (10 mL). The resulting mixture was extracted with EtOAc (3×20 mL). The combined organic layers were washed with brine (3×10 mL), dried over anhydrous Na2SO4. After filtration, the filtrate was concentrated under reduced pressure. The residue was purified by silica gel column chromatography, eluted with PE/EA (5:1) to afford tert-butyl 7-[2-(benzyloxy)ethyl]-2-bromo-4-oxo-1H,6H,7H-pyrrolo[3,2-c]pyridine-5-carboxylate (1200 mg, 83.86%) as a white oil.

LC-MS: M+H found: 449.0.

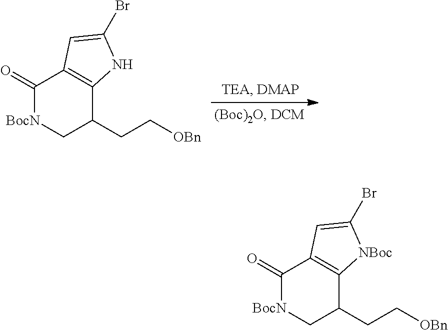 [SMILES: CC(C)(C)OC(=O)N1CC(CCOCc2ccccc2)c2[nH]c(Br)cc2C1=O ; CC(C)(C)OC(=O)N1CC(CCOCc2ccccc2)c2c(cc(Br)n2C(=O)OC(C)(C)C)C1=O]

A solution of tert-butyl 7-[2-(benzyloxy)ethyl]-2-bromo-4-oxo-1H,6H,7H-pyrrolo[3,2-c]pyridine-5-carboxylate (500 mg, 1.11 mmol, 1.00 equiv) and (Boc)₂O (267 mg, 1.224 mmol, 1.1 equiv) and TEA (135 mg, 1.34 mmol, 1.2 equiv) in DCM (5.00 mL) was stirred for 2 h at room temperature under nitrogen atmosphere. The reaction was monitored by LCMS. Desired product could be detected by LCMS. The resulting mixture was concentrated under reduced pressure. The residue was purified by Prep-TLC (PE/EA 5:1) to afford 1,5-di-tert-butyl 7-[2-(benzyloxy)ethyl]-2-bromo-4-oxo-6H,7H-pyrrolo[3,2-c]pyridine-1,5-dicarboxylate (590 mg, 92.64%) as a white oil.

LC-MS: M+H found: 549.0.

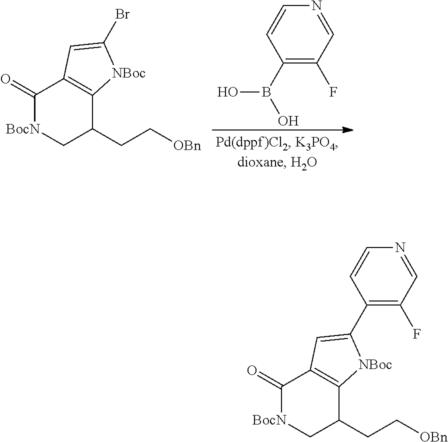 [SMILES: CC(C)(C)OC(=O)N1CC(CCOCc2ccccc2)c2c(cc(Br)n2C(=O)OC(C)(C)C)C1=O ; OB(O)c1ccncc1F ; CC(C)(C)OC(=O)N1CC(CCOCc2ccccc2)c2c(cc(-c3ccncc3F)n2C(=O)OC(C)(C)C)C1=O]

A solution of 1,5-di-tert-butyl 7-[2-(benzyloxy)ethyl]-2-bromo-4-oxo-6H,7H-pyrrolo[3,2-c]pyridine-1,5-dicarboxylate (6.5 g, 11.83 mmol, 1.00 equiv) and 3-fluoropyridin-4-ylboronic acid (2.50 g, 17.74 mmol, 1.5 equiv) and Pd(dppf)Cl₂·CH₂Cl₂ (964 mg, 1.18 mmol, 0.1 equiv) and K₃PO₄ (7.53 g, 35.49 mmol, 3 equiv) in dioxane (100 mL) and H2O (20 mL) was stirred for 1 h at 50 degrees C. under nitrogen atmosphere. The reaction was monitored by LCMS. Desired product could be detected by LCMS. The resulting mixture was concentrated under vacuum. The aqueous layer was extracted with EtOAc (4×50 mL). The residue was purified by silica gel column chromatography, eluted with PE/EA (10:1) to afford 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (5.6 g, 82.02%) as a yellow oil.

LC-MS: M+H found: 566.0.

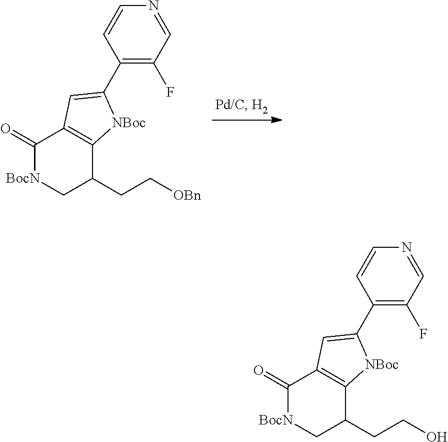 [SMILES: CC(C)(C)OC(=O)N1CC(CCOCc2ccccc2)c2c(cc(-c3ccncc3F)n2C(=O)OC(C)(C)C)C1=O ; CC(C)(C)OC(=O)N1CC(CCO)c2c(cc(-c3ccncc3F)n2C(=O)OC(C)(C)C)C1=O]

To a solution of 1,5-di-tert-butyl 7-[2-(benzyloxy)ethyl]-2-(3-fluoropyridin-4-yl)-4-oxo-6H,7H-pyrrolo[3,2-c]pyridine-1,5-dicarboxylate (4 g, 7.072 mmol, 1.00 equiv) in 140 mL MeOH was added dry Pd/C (10%, 1.6 g) under nitrogen atmosphere in a 500 mL round-bottom flask. The mixture was hydrogenated at room temperature for 2 days under hydrogen atmosphere using a hydrogen balloon, filtered through a Celite pad and concentrated under reduced pressure. The reaction was monitored by LCMS. Desired product could be detected by LCMS. The resulting mixture was filtered, the filter cake was washed with MeOH (3×50 mL). The filtrate was concentrated under reduced pressure to afford 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (2.27 g, 57.38%) as a white oil.

LC-MS: M+H found: 476.0.

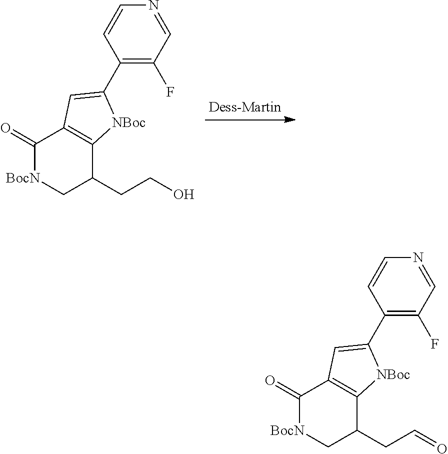 [SMILES: CC(C)(C)OC(=O)N1CC(CCO)c2c(cc(-c3ccncc3F)n2C(=O)OC(C)(C)C)C1=O ; CC(C)(C)OC(=O)N1CC(CC=O)c2c(cc(-c3ccncc3F)n2C(=O)OC(C)(C)C)C1=O]

To a stirred solution of 1,5-di-tert-butyl 2-(3-fluoropyridin-4-yl)-7-(2-hydroxyethyl)-4-oxo-6H,7H-pyrrolo[3,2-c]pyridine-1,5-dicarboxylate (500 mg, 1.05 mmol, 1.00 equiv) in DCM (10 mL) were added Dess-Martin (535 mg, 1.26 mmol, 1.20 equiv) in portions at 0 degrees C. under nitrogen atmosphere. The resulting mixture was stirred for 2 h at room temperature under nitrogen atmosphere. The reaction was monitored by LCMS. Desired product could be detected by LCMS. The resulting mixture was concentrated under vacuum. The residue was purified by silica gel column chromatography, eluted with PE/EA (10:1) to afford 1,5-di-tert-butyl 2-(3-fluoropyridin-4-yl)-4-oxo-7-(2-oxoethyl)-6H,7H-pyrrolo[3,2-c]pyridine-1,5-dicarboxylate (235 mg, 37.29%) as a yellow solid.

LC-MS: M+H found: 474.0.

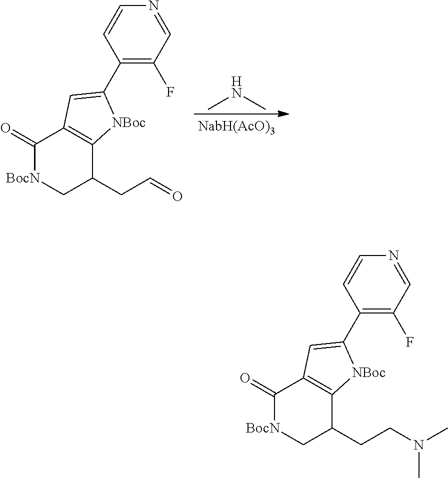

A solution of 1,5-di-tert-butyl 2-(3-fluoropyridin-4-yl)-4-oxo-7-(2-oxoethyl)-6H,7H-pyrrolo[3,2-c]pyridine-1,5-dicarboxylate (170 mg, 0.36 mmol, 1.00 equiv) in DCM (4 mL) was treated with diacetyl peroxide; sodioboranyl acetate (91 mg, 0.43 mmol, 1.20 equiv) for 30 min at room temperature under nitrogen atmosphere followed by the addition of dimethylamine (19 mg, 0.43 mmol, 1.20 equiv) dropwise at room temperature. The resulting mixture was stirred for 2 h at room temperature under nitrogen atmosphere. The reaction was monitored by LCMS. Desired product could be detected by LCMS. The resulting mixture was diluted with water (10 mL). The aqueous layer was extracted with CH₂Cl₂ (2×10 mL). The resulting mixture was concentrated under reduced pressure. The crude product was used in the next step directly without further purification.

LC-MS: M+H found: 503.0.

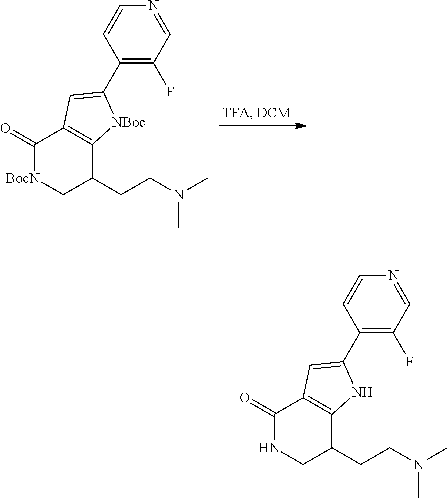 [SMILES: CN(C)CCC1CN(C(=O)OC(C)(C)C)C(=O)c2cc(-c3ccncc3F)n(C(=O)OC(C)(C)C)c21 ; CN(C)CCC1CNC(=O)c2cc(-c3ccncc3F)[nH]c21]

To a stirred mixture of 1,5-di-tert-butyl 2-(3-fluoropyridin-4-yl)-7-[2-(morpholin-4-yl)ethyl]-4-oxo-6H,7H-pyrrolo[3,2-c]pyridine-1,5-dicarboxylate (170 mg, 0.34 mmol, 1.00 equiv) in DCM (6 mL) was added TFA (2 mL) at room temperature under nitrogen atmosphere. The resulting mixture was stirred for 1 h and concentrated under reduced pressure. The residue was purified by reverse flash chromatography with the following conditions: column, C18 silica gel; mobile phase, ACN in water, 10% to 50% gradient in 10 min; detector, UV 254 nm to afford 7-[2-(dimethylamino)ethyl]-2-(3-fluoropyridin-4-yl)-1H,5H,6H,7H-pyrrolo[3,2-c]pyridin-4-one (100 mg, 97.78%) as a yellow solid.

LC-MS: (M+H)⁺ found: 303.

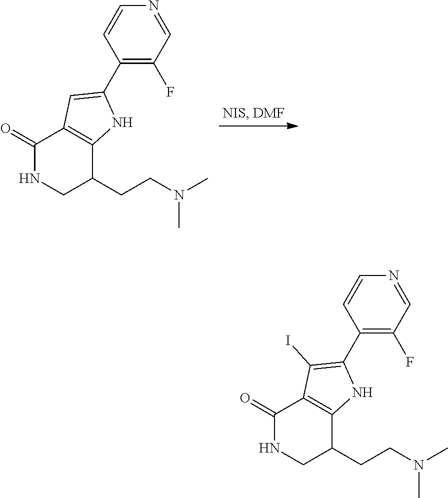 [SMILES: CN(C)CCC1CNC(=O)c2cc(-c3ccncc3F)[nH]c21 ; CN(C)CCC1CNC(=O)c2c1[nH]c(-c1ccncc1F)c2I]

A solution of 7-[2-(dimethylamino)ethyl]-2-(3-fluoropyridin-4-yl)-1H,5H,6H,7H-pyrrolo[3,2-c]pyridin-4-one (130 mg, 0.430 mmol, 1.00 equiv) and NIS (96.73 mg, 0.430 mmol, 1 equiv) in DMF (4 mL) was stirred for 2 h at room temperature under nitrogen atmosphere. The reaction was monitored by LCMS. Desired product could be detected by LCMS. The residue was purified by reverse flash chromatography with the following conditions: column, C18 silica gel; mobile phase, ACN in water, 10% to 50% gradient in 10 min; detector, UV 254 nm to afford 7-[2-(dimethylamino)ethyl]-2-(3-fluoropyridin-4-yl)-3-iodo-1H,5H,6H,7H-pyrrolo[3,2-c]pyridin-4-one (130 mg, 70.60%) as a yellow solid.

LC-MS: M+H found: 429.0.

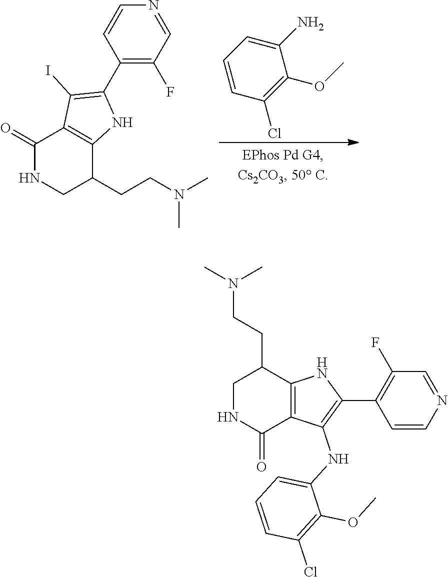

To a stirred mixture of 7-[2-(dimethylamino)ethyl]-2-(3-fluoropyridin-4-yl)-3-iodo-1H,5H,6H,7H-pyrrolo[3,2-c]pyridin-4-one (110 mg, 0.26 mmol, 1.00 equiv) and 3-chloro-2-methoxyaniline (40 mg, 0.26 mmol, 1.00 equiv) in DMF (2.5 mL) were added EPhos Pd G4 (24 mg, 0.026 mmol, 0.1 equiv) and Cs₂CO₃ (167 mg, 0.51 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by reverse flash chromatography with the following conditions: column, C18 silica gel; mobile phase, ACN in water, 10% to 50% gradient in 10 min; detector, UV 254 nm to afford 3-[(3-chloro-2-methoxyphenyl)amino]-7-[2-(dimethylamino)ethyl]-2-(3-fluoropyridin-4-yl)-1H,5H,6H,7H-pyrrolo[3,2-c]pyridin-4-one (70 mg, 59.51%) as a yellow solid.

LC-MS: M+H found: 458.0.

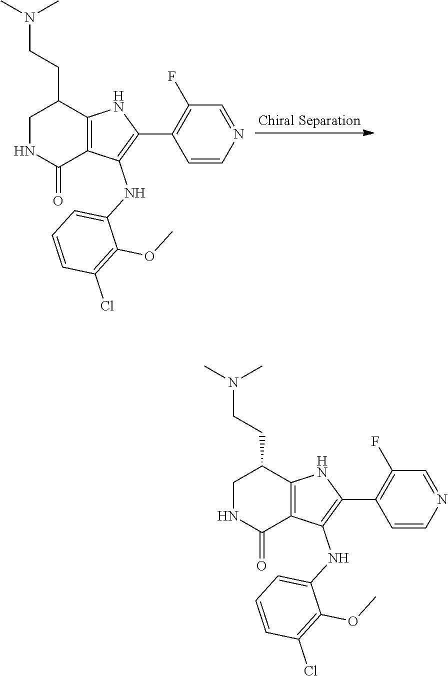 [SMILES: COc1c(Cl)cccc1Nc1c(-c2ccncc2F)[nH]c2c1C(=O)NCC2CCN(C)C ; COc1c(Cl)cccc1Nc1c(-c2ccncc2F)[nH]c2c1C(=O)NC[C@@H]2CCN(C)C]

The crude product (80 mg) was purified by Prep-CHIRAL-HPLC with the following conditions (Column: CHIRALPAK IG-3, 4.6*50 mm, 3 um; Mobile Phase A: Hex (0.1% DEA): EtOH=70: 30; Flow rate: 1 mL/min; Gradient: 0% B to 0% B; Injection Volume: 5 ul mL) to afford (7S)-3-[(3-chloro-2-methoxyphenyl)amino]-7-[2-(dimethylamino)ethyl]-2-(3-fluoropyridin-4-yl)-1H,5H,6H,7H-pyrrolo[3,2-c]pyridin-4-one (18.6 mg, 22.39%) as a white solid.

LCMS (M+H)⁺ found: 458.

¹H NMR (400 MHz, DMSO-d6) δ 12.10 (s, 1H), 8.59 (t, J=1.9 Hz, 1H), 8.33 (d, J=2.7 Hz, 1H), 7.72 (m, 1H), 7.38 (s, 1H), 7.13 (t, J=2.7 Hz, 1H), 6.80-6.63 (m, 2H), 6.16 (m, 1H), 3.89 (s, 3H), 3.48 (m, 1H), 3.20 (m. 1H), 3.09-2.96 (m, 1H), 2.48-2.38 (m, 1H), 2.32 (m, 1H), 2.23 (s, 6H), 1.97 (m, 1H), 1.73-1.55 (m, 1H).

### Example 199. 3-[(3-chloro-2-methoxyphenyl)amino]-2-{2-[2-(dimethylamino)ethoxy]pyrido[3,2-d]pyrimidin-8-yl}-1H,5H,6H,7H-pyrrolo[3,2-c]pyridin-4-one (compound 502)

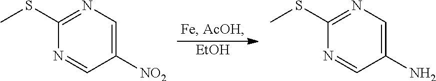

To a stirred solution of 2-(methylsulfanyl)-5-nitropyrimidine (5 g, 29.211 mmol, 1.00 equiv) in EtOH (200 mL) was added AcOH (120 mL) and Fe (17 g, 292.11 mmol, 10 equiv) in portions at rt under N₂ atmosphere. The resulting mixture was stirred for 2 h at 80 degrees C. under N₂ atmosphere. Desired product could be detected by LCMS. LC-MS: M+H found: 142. The resulting mixture was concentrated under reduced pressure. The resulting mixture was extracted with EA (3×100 mL). The combined organic layers were washed with Saturated NaCl (3×100 mL), dried over anhydrous Na₂SO₄. After filtration, the filtrate was concentrated under reduced pressure to afford 2-(methylsulfanyl)pyrimidin-5-amine (3.5 g, 84.86%) as a yellow solid.

LC-MS: M+H found: 142.0.

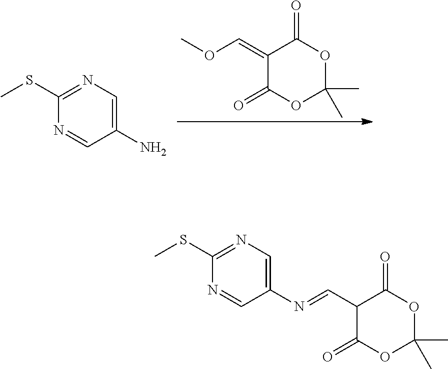

To a stirred solution of 2-(methylsulfanyl)pyrimidin-5-amine (3.2 g, 22.66 mmol, 1.00 equiv) and 5-(methoxymethylidene)-2,2-dimethyl-1,3-dioxane-4,6-dione (5.06 g, 27.18 mmol, 1.20 equiv) in DMF (80.00 mL) at rt under N2 atmosphere. The resulting mixture was stirred for 1 h at 80 degrees C. under N2 atmosphere. Desired product could be detected by LCMS. LC-MS: M+H found: 296. The resulting mixture was added MeOH (50 mL). The resulting mixture was filtered, the filter cake was washed with MeOH (3×20 mL). The filter cake was concentrated under reduced pressure to afford 2,2-dimethyl-5-[(1E)-[[2-(methylsulfanyl)pyrimidin-5-yl]imino]methyl]-1,3-dioxane-4,6-dione (5.4 g, 80.68%) as a yellow solid.

LC-MS: (M+H)⁺ found: 296.0.

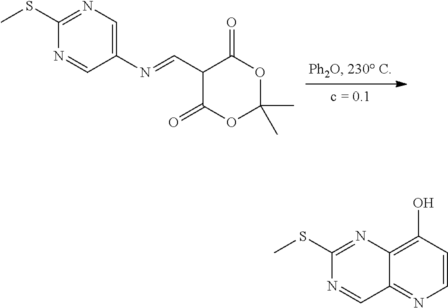

To a stirred solution of 2,2-dimethyl-5-[(1E)-([2-(methylsulfanyl)pyrimidin-5-yl]imino}methyl]-1,3-dioxane-4,6-dione (5.3 g, 17.95 mmol, 1.00 equiv) in phenoxybenzene (360 mL) at rt under N₂ atmosphere. The resulting mixture was stirred at 230 degrees C. under N₂ atmosphere. Desired product could be detected by LCMS. LC-MS: M+H found: 194. The reaction was addition of Hexane (700 ml) at rt. The resulting mixture was filtered; the filter cake was washed with Hexane (3×200 ml). The filtrate was concentrated under reduced pressure.

LC-MS: (M+H)⁺ found: 194.0.

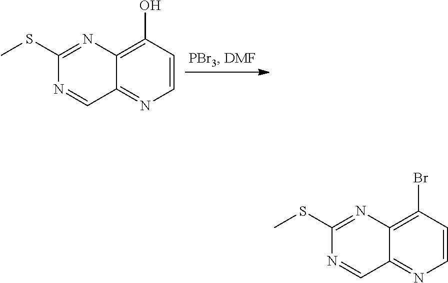

To a stirred solution of 2-(methylsulfanyl)pyrido[3,2-d]pyrimidin-8-ol (2.8 g, 14.49 mmol, 1.00 equiv) in DMF (80 mL) was added PBr₃ (4.31 g, 15.94 mmol, 1.1 equiv) dropwise at 0 degrees C. under N2 atmosphere. The resulting mixture was stirred for 1 h at rt under N2 atmosphere. Desired product could be detected by LCMS. LC-MS: M+H found: 256. The resulting mixture was extracted with EA (3×100 mL). The combined organic layers were washed with aq. NaCl (3×100 mL), dried over anhydrous Na₂SO₄. After filtration, the filtrate was concentrated under reduced pressure. The residue was purified by silica gel column chromatography, eluted with PE:EA (4:1) to afford 8-bromo-2-(methylsulfanyl)pyrido[3,2-d]pyrimidine (1.6 g, 43.11%) as a white solid.

LC-MS: (M+H)⁺ found: 256.0.

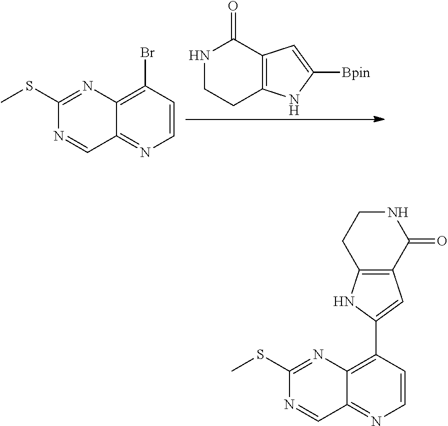

To a stirred solution of 8-bromo-2-(methylsulfanyl)pyrido[3,2-d]pyrimidine (700 mg, 2.73 mmol, 1.00 equiv) and 2-(4,4,5,5-tetramethyl-1,3,2-dioxaborolan-2-yl)-1H,5H,6H,7H-pyrrolo[3,2-c]pyridin-4-one (1075 mg, 4.10 mmol, 1.50 equiv) in dioxane (10 mL) and H₂O (1 mL) were added Na₂CO₃ (869.03 mg, 8.199 mmol, 3.00 equiv) and XPhos Pd G2 (215 mg, 0.27 mmol, 0.10 equiv) dropwise/in portions at rt under N₂ atmosphere. The resulting mixture was stirred for 2 h at 80 degrees C. under N₂ atmosphere. Desired product could be detected by LCMS. LC-MS: M+H found: 312. The resulting mixture was concentrated under vacuum. The residue was purified by silica gel column chromatography, eluted with DCM/MeOH=24:1 to afford 2-[2-(methylsulfanyl)pyrido[3,2-d]pyrimidin-8-yl]-1H,5H,6H,7H-pyrrolo[3,2-c]pyridin-4-one (700 mg, 82.26%) as a yellow solid.

LC-MS: (M+H)⁺ found: 312.0.

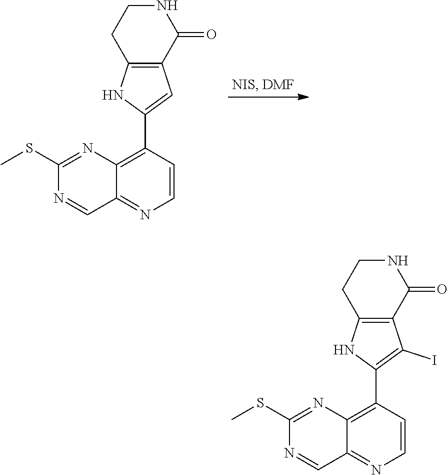 [SMILES: CSc1ncc2nccc(-c3cc4c([nH]3)CCNC4=O)c2n1 ; CSc1ncc2nccc(-c3[nH]c4c(c3I)C(=O)NCC4)c2n1]

To a stirred solution of 2-[2-(methylsulfanyl)pyrido[3,2-d]pyrimidin-8-yl]-1H,5H,6H,7H-pyrrolo[3,2-c]pyridin-4-one (600 mg, 1.93 mmol, 1.00 equiv) and NIS (650.33 mg, 2.891 mmol, 1.50 equiv) in DMF (10 mL) at rt under N2 atmosphere. The resulting mixture was stirred for overnight at 30 degrees C. under N2 atmosphere. Desired product could be detected by LCMS. LC-MS: M+H found: 438. The reaction was quenched by the addition of Saturated aq. Na₂SO₃ (20 mL) at 0 degrees C. The precipitated solids were collected by filtration and washed with H₂O (20 mL×3). The residue was purified by silica gel column chromatography, eluted with DCM:MeOH=10:1 to afford 3-iodo-2-[2-(methylsulfanyl)pyrido[3,2-d]pyrimidin-8-yl]-1H,5H,6H,7H-pyrrolo[3,2-c]pyridin-4-one (650 mg, 77.14%) as a yellow solid.

LC-MS: (M+H)⁺ found: 438.0.

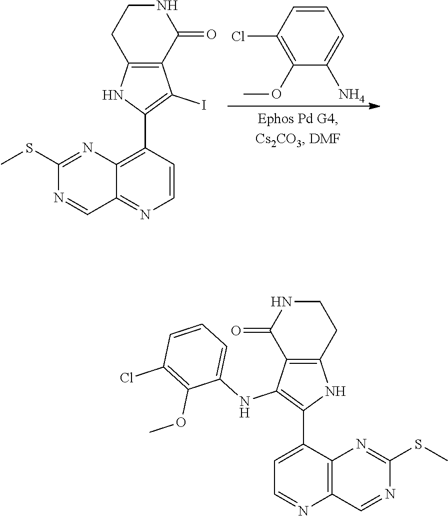

To a stirred solution of 3-iodo-2-[2-(methylsulfanyl)pyrido[3,2-d]pyrimidin-8-yl]-1H,5H,6H,7H-pyrrolo[3,2-c]pyridin-4-one (370 mg, 0.85 mmol, 1.00 equiv) and 3-chloro-2-methoxyaniline (134 mg, 0.85 mmol, 1 equiv) in DMF (4 mL) were added EPhos Pd G4 (78 mg, 0.08 mmol, 0.1 equiv) and Cs₂CO₃ (827 mg, 2.54 mmol, 3 equiv) in portions at rt under Ar atmosphere. The resulting mixture was stirred for 1.5 h at 50 degrees C. under Ar atmosphere. Desired product could be detected by LCMS. LC-MS: M+H found: 467. The resulting mixture was concentrated under vacuum. The residue was purified by silica gel column chromatography, eluted with DCM:MeOH (10:1) to afford 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (220 mg, 55.68%) as a orange solid.

LC-MS: (M+H)⁺ found: 467.0.

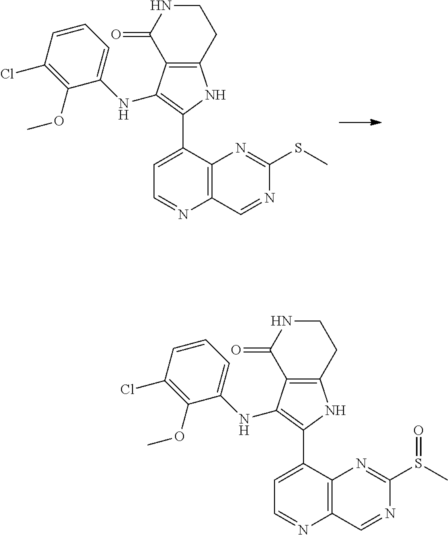 [SMILES: COc1c(Cl)cccc1Nc1c(-c2ccnc3cnc(SC)nc23)[nH]c2c1C(=O)NCC2 ; COc1c(Cl)cccc1Nc1c(-c2ccnc3cnc(S(C)=O)nc23)[nH]c2c1C(=O)NCC2]

To a stirred solution of 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (50 mg, 0.11 mmol, 1.00 equiv) in DCM (2 mL, 31.46 mmol, 293.80 equiv) were added MCPBA (29 mg, 0.12 mmol, 1.1 equiv) dropwise/in portions at 0 degrees C. under N2 atmosphere. The resulting mixture was stirred for 1 h at 0 degrees C. under N2 atmosphere.

Desired product could be detected by LCMS. LC-MS: M+H found: 483. The resulting mixture was extracted with DCM (3×4 mL). The combined organic layers were dried over anhydrous Na₂SO₄. After filtration, the filtrate was concentrated under reduced pressure. The crude product was used in the next step directly without further purification.

LC-MS: (M+H)⁺ found: 483.0.

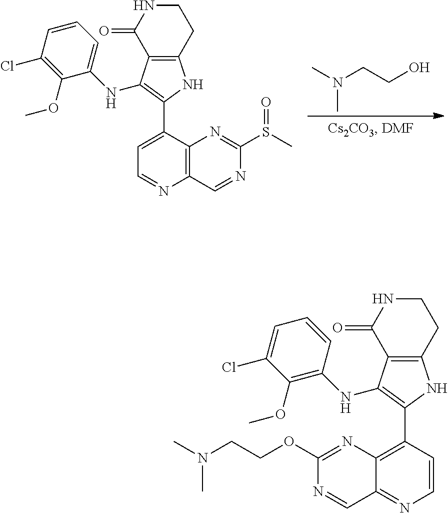

To a stirred solution of 3-[(3-chloro-2-methoxyphenyl)amino]-2-{2-methanesulfinylpyrido[3,2-d]pyrimidin-8-yl}-1H,5H,6H,7H-pyrrolo[3,2-c]pyridin-4-one (145 mg, 0.30 mmol, 1.00 equiv) and Cs₂CO₃ (235 mg, 0.72 mmol, 2.4 equiv) in DMF (2 mL) were added dimethylaminoethanol (33 mg, 0.36 mmol, 1.2 equiv) dropwise at 0 degrees C. under N2 atmosphere. The resulting mixture was stirred for 1 h at RT under N2 atmosphere. The resulting mixture was concentrated under vacuum. The resulting mixture was concentrated under vacuum and dissolved in DMF. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 27% B to 42% B in 11 min, 42% B; Wave Length: 254/220 nm; RT1 (min): 9.67) to 3-[(3-chloro-2-methoxyphenyl)amino]-2-{2-[2-(dimethylamino)ethoxy]pyrido[3,2-d]pyrimidin-8-yl}-1H,5H,6H,7H-pyrrolo[3,2-c]pyridin-4-one (6.1 mg, 4.00%) as a yellow solid.

LC-MS: (M+H)⁺ found: 508.30.

¹H NMR (400 MHz, DMSO-d₆) δ 11.95 (s, 1H), 9.50 (s, 1H), 8.70 (d, J=4.8 Hz, 1H), 7.87 (s, 1H), 7.58 (d, J=4.8 Hz, 1H), 7.27 (d, J=2.6 Hz, 1H), 6.72 (m, J=8.0, 1.7 Hz, 1H), 6.66 (t, J=8.0 Hz, 1H), 6.16 (m, J=8.0, 1.7 Hz, J H), 4.70 (t, J=5.9 Hz, 2H), 3.89 (s, 3H), 3.43-3.51 (m, 2H), 2.95 (t, J=6.8 Hz, 2H), 2.80 (s, 2H), 2.29 (s, 6H).

### Example 200. 3-[(3-chloro-2-methoxyphenyl)amino]-2-[6-(methoxymethyl)-1,5-naphthyridin-4-yl]-1H,5H,6H,7H-pyrrolo[3,2-c]pyridin-4-one (compound 679)

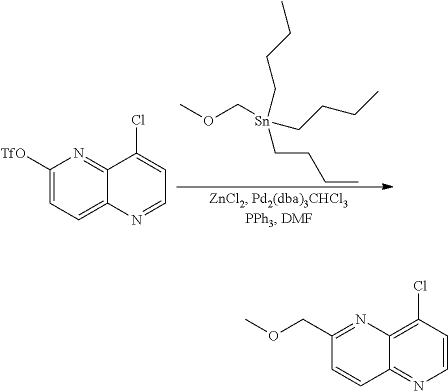

To a stirred mixture of 8-chloro-1,5-naphthyridin-2-yl trifluoromethanesulfonate (100 mg, 0.32 mmol, 1.00 equiv) and tributyl(methoxymethyl)stannane (108 mg, 0.32 mmol, 1 equiv) in DMF (3 mL) were added ZnCl2 (44 mg, 0.32 mmol, 1 equiv) and Pd2(dba)3CHCl3 (33 mg, 0.03 mmol, 0.1 equiv) and PPh₃ (8 mg, 0.03 mmol, 0.1 equiv) in portions at room temperature under argon atmosphere. The resulting mixture was stirred for overnight at 90 degrees C. under argon atmosphere. The reaction was quenched by the addition of sat.KF (aq.) (5 mL) at 0 degrees C. The resulting mixture was extracted with EtOAc (3×10 mL). The combined organic layers were washed with brine (1×5 mL), dried over anhydrous Na2SO4. After filtration, the filtrate was concentrated under reduced pressure. The residue was purified by Prep-TLC (PE/EA 3:1) to afford 8-chloro-2-(methoxymethyl)-1,5-naphthyridine (40 mg, 59.94%) as a yellow solid.

LC-MS: M+H found: 209.

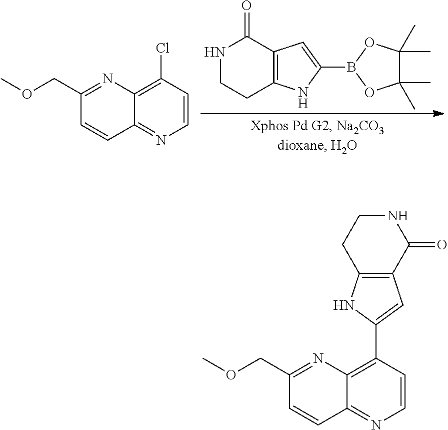

To a stirred mixture of 8-chloro-2-(methoxymethyl)-1,5-naphthyridine (30 mg, 0.144 mmol, 1.00 equiv) and 2-(4,4,5,5-tetramethyl-1,3,2-dioxaborolan-2-yl)-1H,5H,6H,7H-pyrrolo[3,2-c]pyridin-4-one (45 mg, 0.17 mmol, 1.2 equiv) in dioxane (1 mL) and H₂O (0.2 mL) were added XPhos Pd G2 (23 mg, 0.02 mmol, 0.2 equiv) and Na₂CO₃ (30 mg, 0.29 mmol, 2 equiv) in portions at 60 degrees C. under argon atmosphere. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by Prep-TLC (CH₂Cl₂/MeOH 10:1) to afford 2-[6-(methoxymethyl)-1,5-naphthyridin-4-yl]-1H,5H,6H,7H-pyrrolo[3,2-c]pyridin-4-one (40 mg, 90.22%) as a yellow solid.

LC-MS: (M+H)⁺ found: 309.

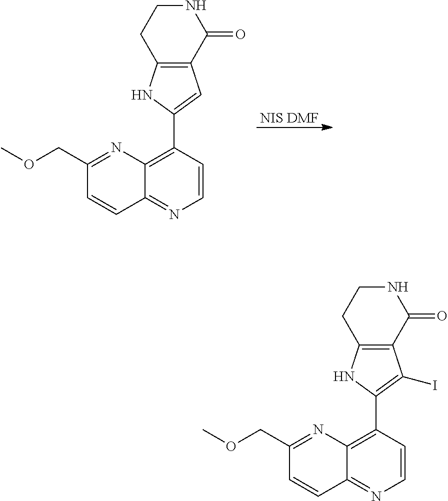

A mixture of 2-[6-(methoxymethyl)-1,5-naphthyridin-4-yl]-1H,5H,6H,7H-pyrrolo[3,2-c]pyridin-4-one (30 mg, 0.10 mmol, 1.00 equiv) and NIS (26 mg, 0.12 mmol, 1.2 equiv) in DMF (1 mL) was stirred for 1 h at room temperature under nitrogen atmosphere. Desired product could be detected by LCMS. The resulting mixture was extracted with EtOAc (3×10 mL). The combined organic layers were washed with brine (1×5 mL), dried over anhydrous Na₂SO₄. After filtration, the filtrate was concentrated under reduced pressure. The residue was purified by Prep-TLC (CH₂Cl₂/MeOH 10:1) to afford 3-iodo-2-[6-(methoxymethyl)-1,5-naphthyridin-4-yl]-1H,5H,6H,7H-pyrrolo[3,2-c]pyridin-4-one (40 mg, 94.68%) as a yellow solid.

LC-MS: (M+H)⁺ found: 435.

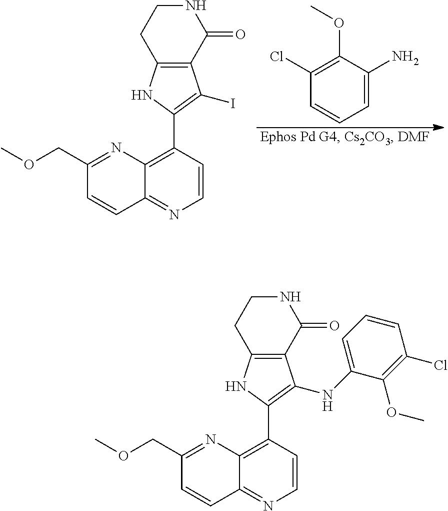

To a stirred mixture of 3-iodo-2-[6-(methoxymethyl)-1,5-naphthyridin-4-yl]-1H,5H,6H,7H-pyrrolo[3,2-c]pyridin-4-one (30 mg, 0.07 mmol, 1.00 equiv) and 3-chloro-2-methoxyaniline (12 mg, 0.08 mmol, 1.10 equiv) in DMF (1.00 mL) were added Ephos Pd G4 (6 mg, 0.01 mmol, 0.1 equiv) and Cs₂CO₃ (45 mg, 0.14 mmol, 2 equiv) in portions at 50 degrees C. under argon atmosphere. The resulting mixture was stirred for 2 h at 50 degrees C. under argon atmosphere. The resulting mixture was concentrated under reduced pressure. The residue was purified by Prep-TLC (CH2Cl2/MeOH 10:1) to afford 3-[(3-chloro-2-methoxyphenyl)amino]-2-[6-(methoxymethyl)-1,5-naphthyridin-4-yl]-1H,5H,6H,7H-pyrrolo[3,2-c]pyridin-4-one (30 mg) as a light yellow solid. The crude product (30 mg) was purified by Prep-HPLC with the following conditions (Column: XSelect CSH Prep C18 OBD Column, 19*250 mm, 5|Ìm; Mobile Phase A: Water (10 mmol/L NH4HCO3), Mobile Phase B: ACN; Flow rate: 25 mL/min; Gradient: 55% B to 57% B in 9 min, 57% B; Wave Length: 254 nm; RT1 (min): 6.00) to afford 3-[(3-chloro-2-methoxyphenyl)amino]-2-[6-(methoxymethyl)-1,5-naphthyridin-4-yl]-1H,5H,6H,7H-pyrrolo[3,2-c]pyridin-4-one (10.6 mg, 33.07%) as a yellow solid.

LC-MS: (M+H)⁺ found: 464.05.

1H NMR (400 MHz, DMSO-d6) δ 12.68 (s, 1H), 8.72 (d, J=4.9 Hz, 1H), 8.44 (d, J=8.7 Hz, 1H), 7.93 (s, 1H), 7.86 (d, J=8.7 Hz, 1H), 7.52 (d, J=4.9 Hz, 1H), 7.23 (d, J=2.8 Hz, 1H), 6.73 (m, J=1.8 Hz, 1H), 6.69 (t, J=8.0 Hz, 1H), 6.20 (m, J=1.8 Hz, 1H), 4.91 (s, 2H), 3.89 (s, 3H), 3.55-3.43 (m, 5H), 2.99 (t, J=6.8 Hz, 2H).

### Example 201. 3-[(3-chloro-2-methoxyphenyl)amino]-2-[6-(2-methoxyethoxy)-1,5-naphthyridin-4-yl]-1H,5H,6H,7H-pyrrolo[3,2-c]pyridin-4-one (compound 678)

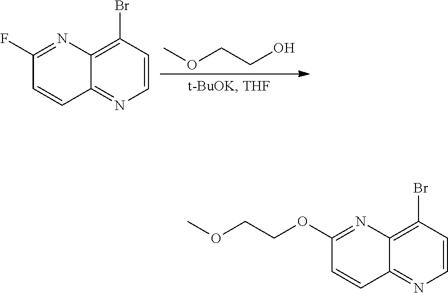

To a solution of 8-bromo-2-fluoro-1,5-naphthyridine (500 mg, 2.202 mmol, 1 equiv) and 2-methoxyethanol (837.92 mg, 11.010 mmol, 5 equiv) in THF (22 mL) was added t-BuOK (741.38 mg, 6.606 mmol, 3 equiv) in portions at 0° C., then the reaction was stirred at 0° C. for 1 hrs. The reaction was monitored by LCMS. The resulting mixture was concentrated under reduced pressure. The residue was purified by silica gel column chromatography, eluted with CH₂Cl₂/MeOH (100:1) to afford 8-bromo-2-(2-methoxyethoxy)-1,5-naphthyridine (640 mg, 98.54%) as a off-white solid.

LC-MS: (M+H)⁺ found: 283.0.

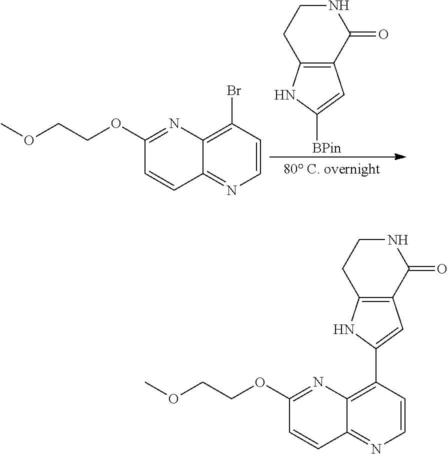

To a stirred mixture of 8-bromo-2-(2-methoxyethoxy)-1,5-naphthyridine (600 mg, 2.119 mmol, 1 equiv) and 2-(4,4,5,5-tetramethyl-1,3,2-dioxaborolan-2-yl)-1H,5H,6H,7H-pyrrolo[3,2-c]pyridin-4-one (833.23 mg, 3.179 mmol, 1.5 equiv) in DME (20 mL) H₂O (0.5 mL, 27.755 mmol, 13.10 equiv) was added Pd(dppf)Cl₂ (155.06 mg, 0.212 mmol, 0.1 equiv), TEA (536.12 mg, 5.298 mmol, 2.5 equiv) at room temperature under argon atmosphere. The resulting mixture was stirred for overnight at 80° C. under argon atmosphere. The reaction was monitored by LCMS. The resulting mixture was extracted with EtOAc (3×100 mL). The combined organic layers were washed with water (1×50 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-[6-(2-methoxyethoxy)-1,5-naphthyridin-4-yl]-1H,5H,6H,7H-pyrrolo[3,2-c]pyridin-4-one (620 mg, 83.00%) as a off-white solid.

LC-MS: (M+H)⁺ found: 339.0.

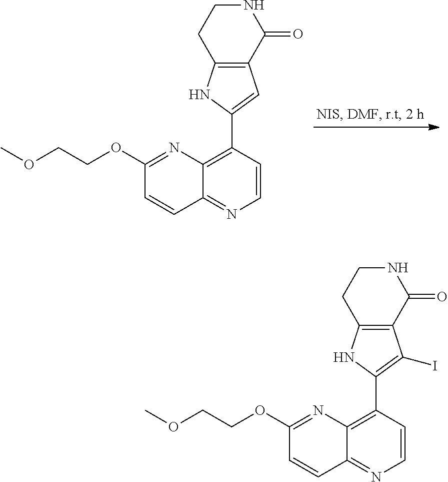 [SMILES: COCCOc1ccc2nccc(-c3cc4c([nH]3)CCNC4=O)c2n1 ; COCCOc1ccc2nccc(-c3[nH]c4c(c3I)C(=O)NCC4)c2n1]

To a stirred mixture of 2-[6-(2-methoxyethoxy)-1,5-naphthyridin-4-yl]-1H,5H,6H,7H-pyrrolo[3,2-c]pyridin-4-one (350 mg, 1.034 mmol, 1 equiv) in DMF (10 mL) was added NIS (279.26 mg, 1.241 mmol, 1.2 equiv) in portions at room temperature under argon atmosphere. The reaction was monitored by LCMS. The resulting mixture was stirred for 2 h at room temperature under argon atmosphere. The reaction was quenched by the addition of sat. sodium sulfite (aq.) (50 mL) at room temperature. The precipitated solids were collected by filtration and washed with water (3×50 mL) to afford 3-iodo-2-[6-(2-methoxyethoxy)-1,5-naphthyridin-4-yl]-1H,5H,6H,7H-pyrrolo[3,2-c]pyridin-4-one (400 mg, 78.71%) as a light yellow solid.

LC-MS: (M+H)⁺ found: 464.85.

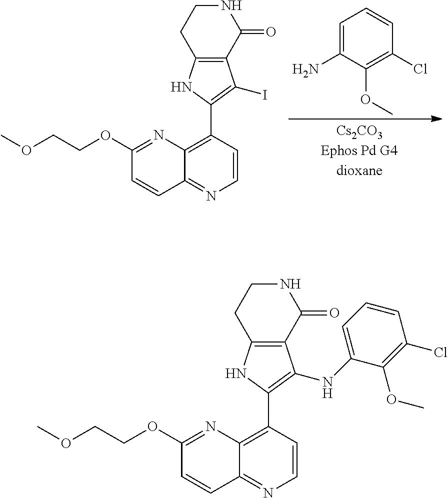

To a stirred mixture of 3-iodo-2-[6-(2-methoxyethoxy)-1,5-naphthyridin-4-yl]-1H,5H,6H,7H-pyrrolo[3,2-c]pyridin-4-one (200.00 mg, 0.430 mmol, 1.00 equiv) in dioxane (4.30 mL) was added 3-chloro-2-methoxyaniline (68.00 mg, 0.430 mmol, 1.00 equiv), Ephos Pd G4 (79.00 mg, 0.086 mmol, 0.20 equiv) and caesio methaneperoxoate caesium (281.00 mg, 0.859 mmol, 2.00 equiv) at room temperature under argon atmosphere. The resulting mixture was stirred for overnight at 50 degrees C. under argon atmosphere. Desired product could be detected by LCMS. The residue was dissolved in ethyl acetate (50 mL). The resulting mixture was washed with 3×10 mL of water. The crude product (mg) was purified by Prep-HPLC with the following conditions ((Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 34% B to 64% B in 7 min; Wave Length: 254 nm; RT1 (min): 6.5) to afford 3-[(3-chloro-2-methoxyphenyl)amino]-2-[6-(2-methoxyethoxy)-1,5-naphthyridin-4-yl]-1H,5H,6H,7H-pyrrolo[3,2-c]pyridin-4-one (33 mg) as a light yellow solid.

LC-MS: (M+H)⁺ found: 494.05.

1H NMR (300 MHz, DMSO-d6) δ 12.09 (s, 1H), 8.57 (d, J=4.8 Hz, 1H), 8.30 (d, J=9.1 Hz, 1H), 7.79 (s, 1H), 7.50 (d, J=4.9 Hz, 1H), 7.36 (d, J=9.1 Hz, 1H), 7.23 (t, J=2.5 Hz, 1H), 6.79-6.57 (m, 2H), 6.18 (dd, J=7.5, 2.2 Hz, 1H), 4.82-4.66 (m, 2H), 3.88 (s, 3H), 3.84-3.75 (m, 2H), 3.47 (td, J=6.8, 2.4 Hz, 2H), 3.35 (s, 3H), 2.95 (t, J=6.8 Hz, 2H).

### Example 202. 3-[(3-chloro-2-methoxyphenyl)amino]-2-(6-cyclopropoxy-1,5-naphthyridin-4-yl)-1H,5H,6H,7H-pyrrolo[3,2-c]pyridin-4-one (compound 677)

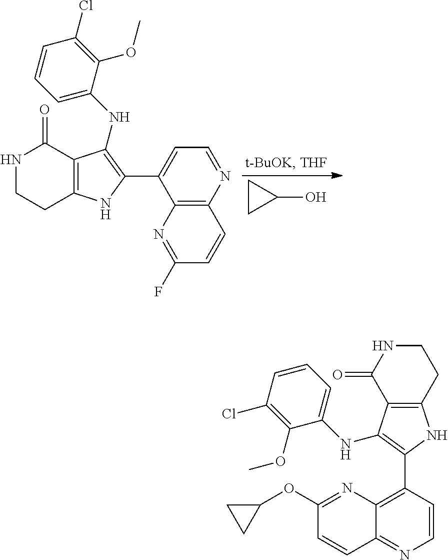

To a stirred solution of 3-[(3-chloro-2-methoxyphenyl)amino]-2-(6-fluoro-1,5-naphthyridin-4-yl)-1H,5H,6H,7H-pyrrolo[3,2-c]pyridin-4-one (200.00 mg, 0.457 mmol, 1.00 equiv) and cyclopropanol (132.65 mg, 2.284 mmol, 5.00 equiv) in THF (5.00 mL) was added t-BuOK (56.38 mg, 0.502 mmol, 1.10 equiv) dropwise at 0 degrees C. under nitrogen atmosphere. The resulting mixture was stirred overnight at room temperature under nitrogen atmosphere. The residue was purified by reverse flash chromatography with the following conditions: column, C18 silica gel; mobile phase, MeCN in water, 10% to 70% gradient in 30 min; detector, UV 254 nm. This resulted in 3-[(3-chloro-2-methoxyphenyl)amino]-2-(6-cyclopropoxy-1,5-naphthyridin-4-yl)-1H,5H,6H,7H-pyrrolo[3,2-c]pyridin-4-one (44.1 mg, 19.44%) as a yellow solid.

LC-MS: (M+H)⁺ found 476.25.

¹H NMR (400 MHz, DMSO-d6) δ 12.73 (s, 1H), 8.51-8.50 (d, J=5.0 Hz, 1H), 8.26-8.23 (d, J=9.1 Hz, 1H), 7.74 (s, 1H), 7.44-7.43 (d, J=4.9 Hz, 1H), 7.28-7.26 (d, J=9.1 Hz, 1H), 7.20 (s, 1H), 6.71-6.64 (m, 2H), 6.12-6.10 (dd, J=7.7, 2.0 Hz, 1H), 4.52-4.50 (dt, J=6.0, 3.0 Hz, 1H), 3.87 (s, 3H), 3.73-3.40 (dt, J=7.0, 3.6 Hz, 2H), 2.81-2.97 (t, J=6.8 Hz, 2H), 0.82-0.90 (ddd, J=15.2, 6.0, 3.8 Hz, 4H).

### Example 203. 3-[(3-fluoro-2-methoxyphenyl)amino]-2-(6-methoxy-1,5-naphthyridin-4-yl)-1H,5H,6H,7H-pyrrolo[3,2-c]pyridin-4-one (compound 681)

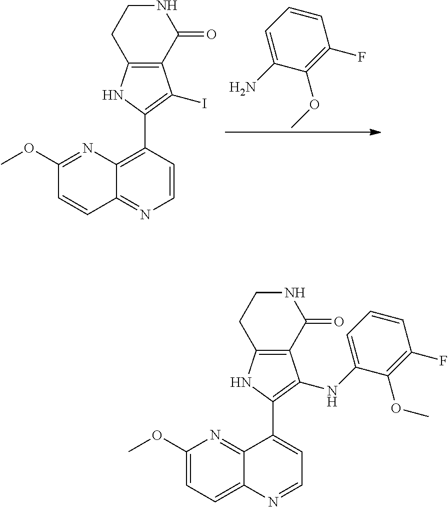

To a stirred mixture of 3-iodo-2-(6-methoxy-1,5-naphthyridin-4-yl)-1H,5H,6H,7H-pyrrolo[3,2-c]pyridin-4-one (60 mg, 0.14 mmol, 1.00 equiv) and 3-fluoro-2-methoxyaniline (25 mg, 0.17 mmol, 1.20 equiv) in dioxane (2.5 mL) were added Cs₂CO₃ (93 mg, 0.28 mmol, 2.00 equiv), EPhos (8 mg, 0.01 mmol, 0.10 equiv) and EPhos Pd G4 (53 mg, 0.06 mmol, 0.40 equiv) under argon atmosphere. The resulting suspension was backfilled with argon three times and stirred for overnight at 50° C. LCMS confirmed completion of reaction and desired product was observed. The resulting mixture was filtered through a pad of silica and the filter cake was washed with DCM (2×10 mL). After filtration, the filtrate was concentrated under reduced pressure. 1. The residue was dissolved in DMF (0.5 mL). The crude product (100 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 50 mL/min; Gradient: 35% B to 60%/B in 7 min, 60% B; Wave Length: 254 nm; RT1 (min): 6.2) to afford 3-[(3-fluoro-2-methoxyphenyl)amino]-2-(6-methoxy-1,5-naphthyridin-4-yl)-1H,5H,6H,7H-pyrrolo[3,2-c]pyridin-4-one (5.1 mg, 8.24%) as a yellow solid.

LC-MS: (M+H)⁺ found: 434.05.

¹H NMR (400 MHz, DMSO-d₆) δ 12.12 (s, 1H), 8.59 (d, J=4.9 Hz, 1H), 8.30 (d, J=9.0 Hz, 1H), 7.81 (s, 1H), 7.52 (d, J=4.9 Hz, 1H), 7.36 (d, J=9.1 Hz, 1H), 7.19 (t, J=2.4 Hz, 1H), 6.62 (m, J=8.3, 6.0 Hz, 1H), 6.50 (m, J=11.0, 8.3, 1.5 Hz, 1H), 6.06 (m, J=8.2, 1.3 Hz, 1H), 4.18 (s, 3H), 3.87 (d, J=0.9 Hz, 3H), 3.46 (m, J=6.9, 2.5 Hz, 2H), 2.95 (t, J=6.8 Hz, 2H).

### Example 204. 3-[(3-fluoro-2-methoxyphenyl)amino]-2-(6-methoxy-7-methyl-1,5-naphthyridin-4-yl)-1H,5H,6H,7H-pyrrolo[3,2-c]pyridin-4-one (compound 682)

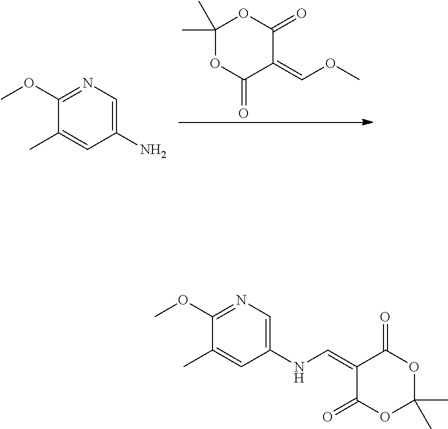

To a stirred solution of 6-methoxy-5-methylpyridin-3-amine (1.00 g, 7.237 mmol, 1.00 equiv) and 5-(methoxymethylidene)-2,2-dimethyl-1,3-dioxane-4,6-dione (1.62 g, 8.685 mmol, 1.20 equiv) in DMF (20.00 mL) at room temperature under nitrogen atmosphere. The resulting mixture was stirred for 1 h at 80 degrees C. under nitrogen atmosphere. The residue was purified by trituration with MeOH (10 mL). This resulted in 5-[[(6-methoxy-5-methylpyridin-3-yl)amino]methylidene]-2,2-dimethyl-1,3-dioxane-4,6-dione (1.50 g, 70.91%) as a yellow solid.

LC-MS: M+H found: 293.10.

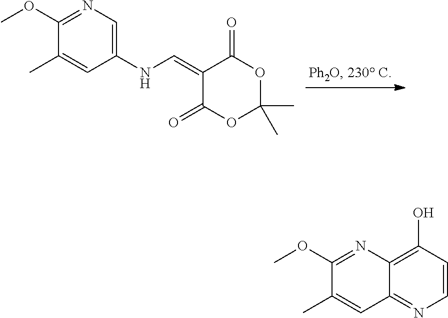

To a stirred solution of 5-[[(6-methoxy-5-methyl-2,3-dihydropyridin-3-yl)amino]methylidene]-2,2-dimethyl-1,3-dioxane-4,6-dione (1.00 g) in diphenyl-ether (15.00 mL) at 240 degrees C. under air atmosphere. The resulting mixture was stirred for 1 h at 240 degrees C. under air atmosphere. The residue was purified by trituration with hexane (10 mL). The precipitated solids were collected by filtration This resulted in 6-methoxy-7-methyl-1,5-naphthyridin-4-ol (800.00 mg) as a brown solid.

LC-MS: M+H found: 191.20.

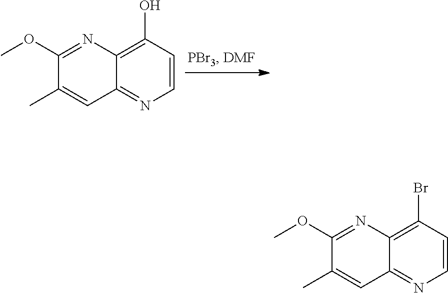

To a stirred solution of 6-methoxy-7-methyl-1,5-naphthyridin-4-ol (800.00 mg, 4.206 mmol, 1.00 equiv) in DMF (10.00 mL) was added PBr3 (1366.22 mg, 0.000 mmol, 1.20 equiv) dropwise at 0 degrees C. under nitrogen atmosphere. The resulting mixture was stirred for 2 h at room temperature under nitrogen atmosphere. The resulting mixture was diluted with ice water (15 mL). The mixture was basified to pH 8 with saturated Na2CO3 (aq.). The precipitated solids were collected by filtration. This resulted in 8-bromo-2-methoxy-3-methyl-1,5-naphthyridine (750.00 mg) as a brown solid.

LC-MS: M+H found: 253.00.

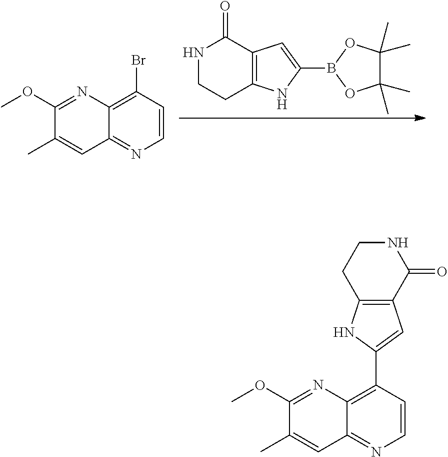

To a stirred solution of 8-bromo-2-methoxy-3-methyl-1,5-naphthyridine (500.00 mg, 1.976 mmol, 1.00 equiv), Cs₂CO₃ (1930.98 mg, 5.928 mmol, 3.00 equiv) and 2-(4,4,5,5-tetramethyl-1,3,2-dioxaborolan-2-yl)-1H,5H,6H,7H-pyrrolo[3,2-c]pyridin-4-one (569.60 mg, 0.000 mmol, 1.10 equiv) in dioxane (4.00 mL) and H2O (1.00 mL) was added Pd(dppf)Cl₂ (144.55 mg, 0.198 mmol, 0.10 equiv) at room temperature under nitrogen atmosphere. The resulting mixture was stirred for 12 h at 95 degrees C. under nitrogen atmosphere. The resulting mixture was diluted with water (20 mL). The aqueous layer was extracted with EtOAc (3×30 mL). The resulting mixture was concentrated under vacuum. The residue was purified by Prep-TLC (CH₂Cl₂/MeOH 20:1) to afford 2-(6-methoxy-7-methyl-1,5-naphthyridin-4-yl)-1H,5H,6H,7H-pyrrolo[3,2-c]pyridin-4-one (200.00 mg, 32.83%) as a yellow solid.

LC-MS: M+H found: 309.10.

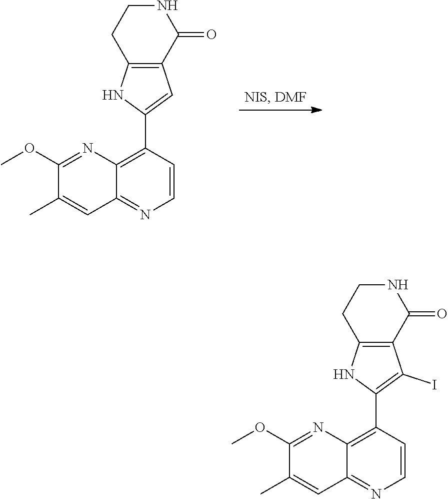 [SMILES: COc1nc2c(-c3cc4c([nH]3)CCNC4=O)ccnc2cc1C ; COc1nc2c(-c3[nH]c4c(c3I)C(=O)NCC4)ccnc2cc1C]

To a stirred solution of 2-(6-methoxy-7-methyl-1,5-naphthyridin-4-yl)-1H,5H,6H,7H-pyrrolo[3,2-c]pyridin-4-one (200.00 mg, 1.297 mmol, 1.00 equiv) in DMF (3 mL) was added NIS (291.86 mg, 1.297 mmol, 1.00 equiv) in portions at room temperature under nitrogen atmosphere. The resulting mixture was stirred for 1 h at room temperature under nitrogen atmosphere. The reaction was quenched with sat. Na2O3S (aq.) 3 mL at 0 degrees C. The product was precipitated by the addition of water. The precipitated solids were collected by filtration This resulted in 3-iodo-2-(6-methoxy-7-methyl-1,5-naphthyridin-4-yl)-1H,5H,6H,7H-pyrrolo[3,2-c]pyridin-4-one (90 mg, 29.81%) as a brown solid.

LC-MS: M+H found: 435.0.

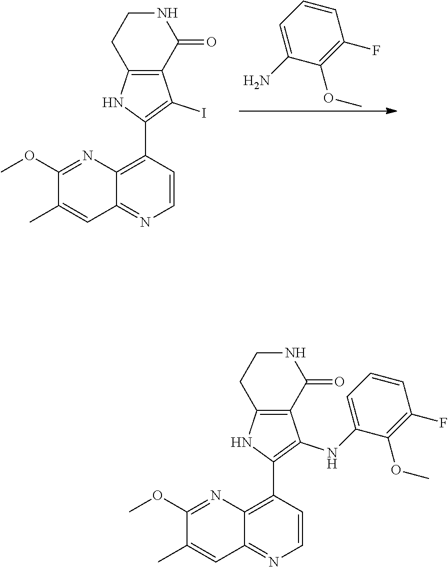

To a stirred solution of 3-iodo-2-(6-methoxy-7-methyl-1,5-naphthyridin-4-yl)-1H,5H,6H,7H-pyrrolo[3,2-c]pyridin-4-one (80.00 mg, 0.184 mmol, 1.00 equiv), Cs₂CO₃ (120.05 mg, 0.368 mmol, 2.00 equiv) and 3-fluoro-2-methoxyaniline (28.60 mg, 0.000 mmol, 1.10 equiv) in dioxane (2.00 mL) was added BrettPhos Pd G3 (33.40 mg, 0.037 mmol, 0.20 equiv) at room temperature under nitrogen atmosphere. The resulting mixture was stirred overnight at 70 degrees C. under nitrogen atmosphere. The crude product (40 mg) was purified by Prep-HPLC with the following conditions (Column: XBridge Prep OBD C18 Column, 30*150 mm, 5 μm; Mobile Phase A. Water (1OMMOL/L NH4HCO3), Mobile Phase B: ACN; Flow rate: 60 mL/min; Gradient: 35% B to 60% B in 8 min; Wave Length: 254 nm: RT1 (min): 6) to afford 3-[(3-fluoro-2-methoxyphenyl)amino]-2-(6-methoxy-7-methyl-1,5-naphthyridin-4-yl)-1H,5H,6H,7H-pyrrolo[3,2-c]pyridin-4-one (1.40 mg) as a light yellow solid.

LC-MS: (M+H)⁺ found: 448.10.

¹H NMR (400 MHz, DMSO-d6) δ 12.15 (s, 1H), 8.54 (d, J=4.9 Hz, 1H), 8.15 (d, J=1.2 Hz, 1H), 7.78 (s, 1H), 7.47 (d, J=4.8 Hz, 1H), 7.18 (s, 1H), 6.62-6.60 (m, 1H), 6.51-6.49 (m, 1H), 6.06 (d, J=8.1 Hz, 1H), 4.21 (s, 3H), 3.85 (s, 3H) 3.44-3.48 (m, 2H), 2.94 (t, J=6.8 Hz, 2H), 2.39 (d, J=1.1 Hz, 3H).

### Example 205. 3-[(3-fluoro-2-methylphenyl)amino]-2-(6-methoxy-1,5-naphthyridin-4-yl)-1H,5H,6H,7H-pyrrolo[3,2-c]pyridin-4-one (compound 684)

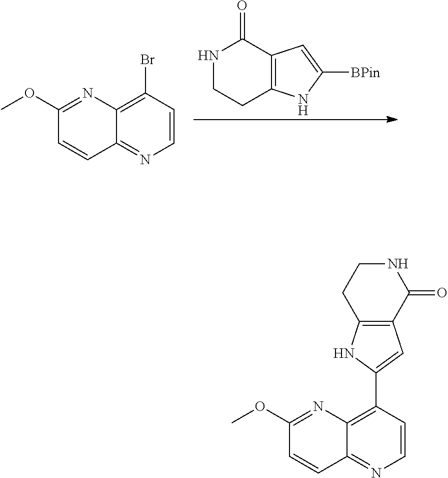

To a stirred solution of 8-bromo-2-methoxy-1,5-naphthyridine (273 mg, 1.14 mmol, 1.00 equiv) and 2-(3,3,4,4-tetramethylborolan-1-yl)-octahydro-1H-pyrrolo[3,2-c]pyridin-4-ol (302 mg, 1.14 mmol, 1.00 equiv) in dioxan (2.5 mL) and H2O (0.5 mL) were added Na2CO3 (363 mg, 3.43 mmol, 3.00 equiv) and Pd(PPh₃)₄ (264 mg, 0.23 mmol, 0.20 equiv) in portions at room temperature under N2 atmosphere. The resulting mixture was stirred for overnight at 50 degrees C. under N2 atmosphere. Desired product could be detected by LCMS. The resulting mixture was extracted with EA (3×10 ml). The combined organic layers were washed with Saturated aq. NaCl (3×20 ml), dried over anhydrous Na₂SO₄. After filtration, the filtrate was concentrated under reduced pressure. The residue was purified by silica gel column chromatography, eluted with DCM/MeOH=10:1 to afford 2-(6-methoxy-1,5-naphthyridin-4-yl)-1H,5H,6H,7H-pyrrolo[3,2-c]pyridin-4-one (70 mg, 20.83%) as a yellow solid.

LC-MS: M+H found: 295.05.

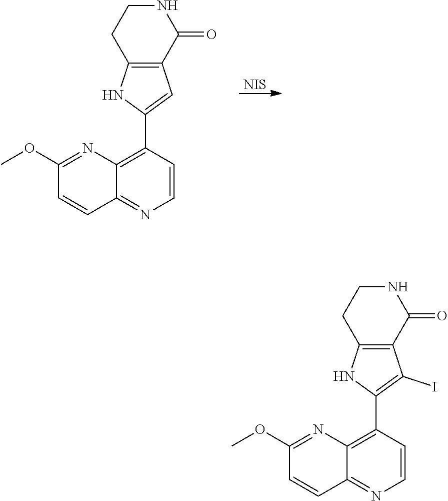

To a stirred solution of 2-(6-methoxy-1,5-naphthyridin-4-yl)-1H,5H,6H,7H-pyrrolo[3,2-c]pyridin-4-one (60 mg, 0.20 mmol, 1.00 equiv) and NIS (46 mg, 0.21 mmol, 1.00 equiv) in DMF (2 mL) at rt under N2 atmosphere. Desired product could be detected by LCMS. By added water, the precipitated solids were collected by filtration and washed with H₂O (3×20 mL) to afford 3-iodo-2-(6-methoxy-1,5-naphthyridin-4-yl)-1H,5H,6H,7H-pyrrolo[3,2-c]pyridin-4-one (50 mg, 58.37%) as a yellow solid.

LC-MS: (M+H)⁺ found: 420.9.

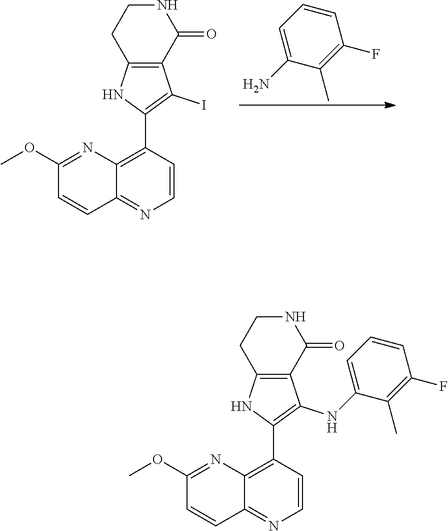

To a stirred mixture of 3-iodo-2-(6-methoxy-1,5-naphthyridin-4-yl)-1H,5H,6H,7H-pyrrolo[3,2-c]pyridin-4-one (60 mg, 0.14 mmol, 1.00 equiv) and 3-fluoro-2-methyl-aniline (21 mg, 0.17 mmol, 1.20 equiv) in dioxane (2.5 mL) were added Cs₂CO₃ (93 mg, 0.28 mmol, 2.00 equiv), EPhos (8 mg, 0.01 mmol, 0.10 equiv) and EPhos Pd G4 (53 mg, 0.06 mmol, 0.40 equiv) under argon atmosphere. The resulting suspension was backfilled with argon three times and stirred for overnight at 50° C. LCMS confirmed completion of reaction and desired product was observed. The resulting mixture was filtered through a pad of silica and the filter cake was washed with DCM (2×10 mL). After filtration, the filtrate was concentrated under reduced pressure. 1. The residue was dissolved in DMF (0.5 mL). The crude product (100 mg) was purified by Prep-HPLC with the following conditions (Column: XBridge Prep OBD C18 Column, 30*150 mm, 5 μm; Mobile Phase A: Water (10mmoL/L NH4HCO3), Mobile Phase B: ACN; Flow rate: 50 mL/min; Gradient: 40% B to 60% B in 7 min, 60% B; Wave Length: 254 nm; RT1 (min): 5.82) to afford 3-[(3-fluoro-2-methylphenyl)amino]-2-(6-methoxy-1,5-naphthyridin-4-yl)-1H,5H,6H,7H-pyrrolo[3,2-c]pyridin-4-one (5.8 mg, 9.73%) as a yellow solid.

LC-MS: (M+H)⁺ found: 418.05.

¹H NMR (400 MHz, DMSO-d6) δ 12.19 (s, 1H), 8.51 (d, J=4.9 Hz, 1H), 8.29 (d, J=9.1 Hz, 1H), 7.55 (s, 1H), 7.44 (d, J=4.9 Hz, 1H), 7.34 (d, J=9.1 Hz, 1H), 7.24 (s, 1H), 6.75 (q, J=7.8 Hz, 1H), 6.49 (t, J=8.8 Hz, 1H), 6.10 (d, J=8.2 Hz, 1H), 4.18 (s, 3H), 3.47 (d, J=2.5 Hz, 2H), 2.96 (t, J=6.8 Hz, 2H), 2.24 (d, J=1.7 Hz, 3H).

### Example 206. 2-(2-amino-3-fluoropyridin-4-yl)-3-[(3-fluoro-2-methoxyphenyl)amino]-1H,5H,6H,7H-pyrrolo[3,2-c]pyridin-4-one (compound 687)

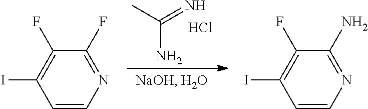

To a stirred solution of 2,3-difluoro-4-iodopyridine (4.60 g, 19.08 mmol, 1.00 equiv) in DMSO (45.00 mL) was added acetamidine (2.19 g, 22.90 mmol, 1.20 equiv) and NaOH (1.91 g, 47.722 mmol, 2.50 equiv) dropwise at room temperature under nitrogen atmosphere. To the above mixture was added H₂O (1.72 g, 95.47 mmol, 5.00 equiv) dropwise at room temperature. The resulting mixture was stirred for additional overnight at 130 degrees C. The reaction was monitored by LCMS. LCMS showed the reaction was completed. The resulting mixture was extracted with EtOAc (3×150 mL). The combined organic layers were washed with brine (3×200 mL), dried over anhydrous Na₂SO₄. After filtration, the filtrate was concentrated under reduced pressure. The residue was purified by silica gel column chromatography, eluted with PE/EtOAc (1:1) to afford 3-fluoro-4-iodopyridin-2-amine (1.725 g, 37.97%) as an off-white solid.

LC-MS: (M+H)⁺ found: 238.85.

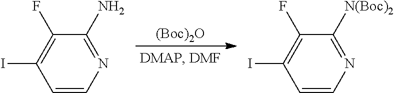

To a stirred solution of 3-fluoro-4-iodopyridin-2-amine (1.70 g, 7.14 mmol, 1.00 equiv) in DMF (35.00 mL) was added DMAP (0.13 g, 1.07 mmol, 0.15 equiv) dropwise at room temperature under nitrogen atmosphere. To the above mixture was added di-tert-butyl dicarbonate (4.68 g, 21.42 mmol, 3.00 equiv) in portions at room temperature. The resulting mixture was stirred for additional 3 h at room temperature. The reaction was monitored by LCMS. LCMS showed the reaction was completed. The resulting mixture was extracted with EtOAc (3×150 mL). The combined organic layers were washed with brine (2×200 mL), dried over anhydrous Na₂SO₄. After filtration, the filtrate was concentrated under reduced pressure. The residue was purified by Prep-TLC (PE/EtOAc 3:1) to afford tert-butyl N-(tert-butoxycarbonyl)-N-(3-fluoro-4-iodopyridin-2-yl)carbamate (2.38 g, 76.03%) as an off-white solid.

LC-MS: (M+H)⁺ found: 438.95.

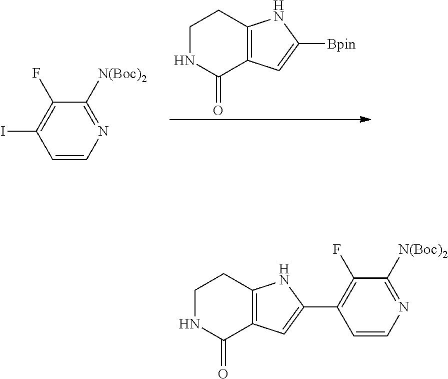

To a stirred solution of tert-butyl N-(tert-butoxycarbonyl)-N-(3-fluoro-4-iodopyridin-2-yl)carbamate (1.00 g, 2.28 mmol, 1.00 equiv) and 2-(4,4,5,5-tetramethyl-1,3,2-dioxaborolan-2-yl)-1H,5H,6H,7H-pyrrolo[3,2-c]pyridin-4-one (1.20 g, 4.57 mmol, 2.00 equiv) in dioxane (20.00 mL) was added Na2CO3 (725 mg, 6.84 mmol, 3.00 equiv) and Pd(PPh₃)₄ (264 mg, 0.22 mmol, 0.10 equiv) in portions at room temperature under nitrogen atmosphere. To the above mixture was added H2O (4.00 mL) dropwise at 50 degrees C. The resulting mixture was stirred for additional overnight at 50 degrees C. The precipitated solids were collected by filtration and washed with EtOAc (3×100 mL). To afford tert-butyl N-(tert-butoxycarbonyl)-N-(3-fluoro-4-[4-oxo-1H,5H,6H,7H-pyrrolo[3,2-c]pyridin-2-yl]pyridin-2-yl)carbamate (600 mg) as a grey solid.

LC-MS: (M+H)⁺ found: 447.

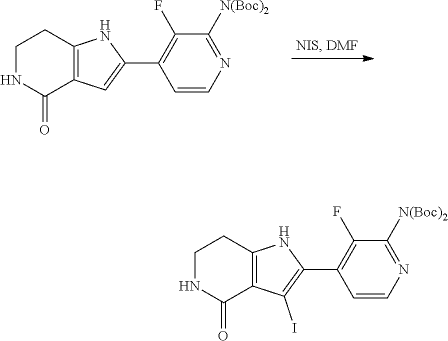

To a stirred solution of tert-butyl N-(tert-butoxycarbonyl)-N-(3-fluoro-4-[4-oxo-1H,5H,6H,7H-pyrrolo[3,2-c]pyridin-2-yl]pyridin-2-yl)carbamate (690 mg, 1.54 mmol, 1.00 equiv) in DMF (15.00 mL) was added N-iodosuccinimide (382 mg, 1.70 mmol, 1.10 equiv) dropwise at room temperature under nitrogen atmosphere. The reaction was monitored by LCMS. LCMS showed the reaction was completed. The reaction was quenched by the addition of Na2SO3 (IOmL) at room temperature. The mixture was neutralized to pH 7 with saturated NaHCO₃ (aq.). The resulting mixture was extracted with CH₂Cl2:MeOH (3×100 mL). The combined organic layers were washed with brine (2×300 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-(tert-butoxycarbonyl)-N-(3-fluoro-4-[3-iodo-4-oxo-1H,5H,6H,7H-pyrrolo[3,2-c]pyridin-2-yl]pyridin-2-yl)carbamate (317 mg, 35.84%) as an off-white solid.

LC-MS: (M+H)⁺ found: 573.1.

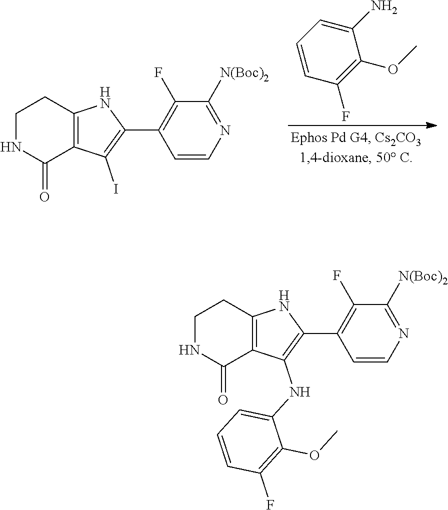

To a stirred mixture of tert-butyl N-(tert-butoxycarbonyl)-N-(3-fluoro-4-[3-iodo-4-oxo-1H,5H,6H,7H-pyrrolo[3,2-c]pyridin-2-yl]pyridin-2-yl)carbamate (270.00 mg, 0.472 mmol, 1.00 equiv) and 3-fluoro-2-methoxyaniline (199.74 mg, 1.416 mmol, 3.00 equiv) in dioxane (2 mL, 0.047 mmol, 0.10 equiv) was added EPhos Pd G4 (86.66 mg, 0.094 mmol, 0.20 equiv) and Cs₂CO₃ (307.39 mg, 0.944 mmol, 2.00 equiv) at room temperature under nitrogen atmosphere. The resulting mixture was stirred for additional 3 h at 50 degrees C. The reaction was monitored by LCMS. LCMS showed the reaction was completed.

The resulting mixture was concentrated under reduced pressure. The residue was purified by silica gel column chromatography, eluted with CH2Cl2/MeOH (10:1) to afford tert-butyl N-(tert-butoxycarbonyl)-N-(3-fluoro-4-[3-[(3-fluoro-2-methoxyphenyl)amino]-4-oxo-1H,5H,6H,7H-pyrrolo[3,2-c]pyridin-2-yl]pyridin-2-yl)carbamate (260 mg, 94.12%) as a yellow solid.

LC-MS: (M+H)⁺ found: 486.15.

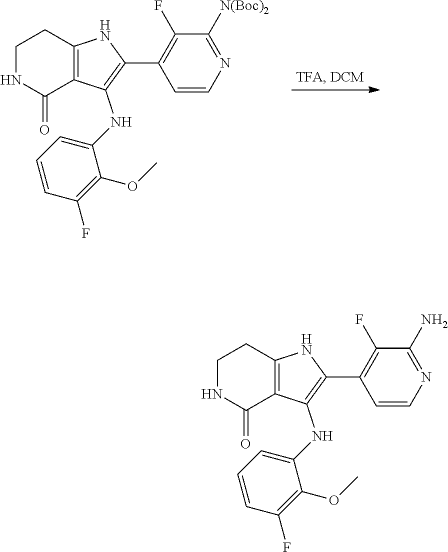

To a stirred solution of tert-butyl N-(tert-butoxycarbonyl)-N-(3-fluoro-4-[3-[(3-fluoro-2-methoxyphenyl)amino]-4-oxo-1H,5H,6H,7H-pyrrolo[3,2-c]pyridin-2-yl]pyridin-2-yl)carbamate (240 mg, 0.41 mmol, 1.00 equiv) in DCM (2.00 mL) was added TEA (2.00 mL) dropwise at 0 degrees C. under nitrogen atmosphere. The resulting mixture was stirred for additional 1 h at room temperature. The reaction was monitored by LCMS. LCMS showed the reaction was completed. The resulting mixture was concentrated under vacuum. The crude product (150 mg) was purified by Prep-HPLC with the following conditions (Column: XBridge Prep OBD C18 Column, 30*150 mm, Sum; Mobile Phase A: Water (1OMMOL/L NH4HCO3), Mobile Phase B: ACN; Flow rate: 60 mL/min; Gradient: 15% B to 45% B in 7 min; Wave Length: 254 nm; RT1 (min): 6.5) to afford 242-amino-3-fluoropyridin-4-yl)-3-[(3-fluoro-2-methoxyphenyl)amino]-1H,5H,6H,7H-pyrrolo[3,2-c]pyridin-4-one (96.5 mg, 61.10%) as a white solid.

LC-MS: (M+H)⁺ found: 386.05.

¹H NMR (400 MHz, DMSO-d6) δ 11.39 (s, 1H), 7.57 (d, J=8.0 Hz, 1H), 7.39 (s, 1H), 7.09 (s, 1H), 6.65-6.58 (m, 2H), 6.48-6.45 (m, 1H), 6.09-6.03 (m, 3H), 3.88 (s, 3H), 3.42-3.38 (m, 2H), 2.83 (t, J=8.0 Hz, 2H).

### Example 207. 2-(2-amino-5-fluoropyrimidin-4-yl)-3-[(3-fluoro-2-methoxyphenyl)amino]-1H,5H,6H,7H-pyrrolo[3,2-c]pyridin-4-one (compound 688)

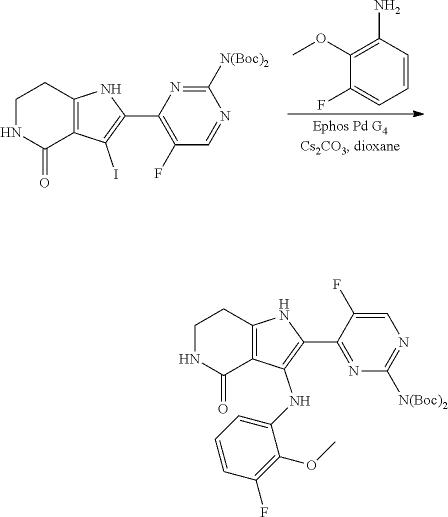

To a stirred mixture of tert-butyl N-(tert-butoxycarbonyl)-N-(5-fluoro-4-[3-iodo-4-oxo-1H,5H,6H,7H-pyrrolo[3,2-c]pyridin-2-yl]pyrimidin-2-yl)carbamate (200 mg, 0.35 mmol, 1.00 equiv) and 3-fluoro-2-methoxyaniline (74 mg, 0.52 mmol, 1.50 equiv) in dioxane (3 mL) were added Ephos Pd G4 (64 mg, 0.07 mmol, 0.2 equiv) and Cs₂CO₃ (227 mg, 0.70 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl N-(tert-butoxycarbonyl)-N-(5-fluoro-4-[3-[(3-fluoro-2-methoxyphenyl)amino]-4-oxo-1H,5H,6H,7H-pyrrolo[3,2-c]pyridin-2-yl]pyrimidin-2-yl)carbamate (87 mg, 42.52%) as a light yellow oil.

LC-MS: M+H found: 587.0.

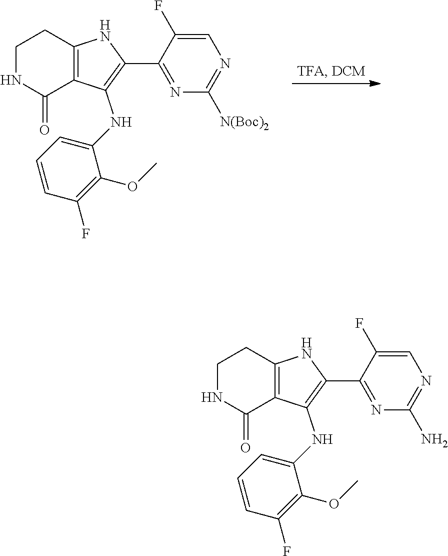

To a stirred mixture of tert-butyl N-(tert-butoxycarbonyl)-N-(5-fluoro-4-[3-[(3-fluoro-2-methoxyphenyl)amino]-4-oxo-1H,5H,6H,7H-pyrrolo[3,2-c]pyridin-2-yl]pyrimidin-2-yl)carbamate (87 mg, 0.15 mmol, 1.00 equiv) in DCM (1.2 mL) and TFA (0.4 mL) was stirred for 1 h at room temperature under nitrogen atmosphere. The resulting mixture was concentrated under reduced pressure. The crude product was purified by Prep-HPLC with the following conditions (Column: Xselect CSH OBD Column 30*150 mm 5 um, n; Mobile Phase A: Water (0. 1% FA), Mobile Phase B: ACN; Flow rate: 60 mL/min; Gradient: 19% B to 30% B in 8 min, 30% B; Wave Length: 254/220 nm; RT1 (min): 7.07) to afford 2-(2-amino-5-fluoropyrimidin-4-yl)-3-[(3-fluoro-2-methoxyphenyl)amino]-1H,5H,6H,7H-pyrrolo[3,2-c]pyridin-4-one (13.7 mg, 23.67%) as a yellow solid.

LC-MS: M+H found: 387.0.

¹H NMR (400 MHz, DMSO-d6) δ 11.45 (s, 1H), 8.57 (s, 1H), 8.13 (d, J=3.4 Hz, 1H), 7.17 (d, J=3.3 Hz, 1H), 6.74 (m, 1H), 6.54 (m, 1H), 6.38-6.21 (m, 3H), 3.90 (s, 3H), 3.42 (m, 2H), 2.85 (t, J=6.7 Hz, 2H).

### Example 208. 2-(2-amino-5-fluoropyrimidin-4-yl)-3-[(3-fluoro-2-methoxyphenyl)amino]-1H,5H,6H,7H-pyrrolo[3,2-c]pyridin-4-one (compound 689)

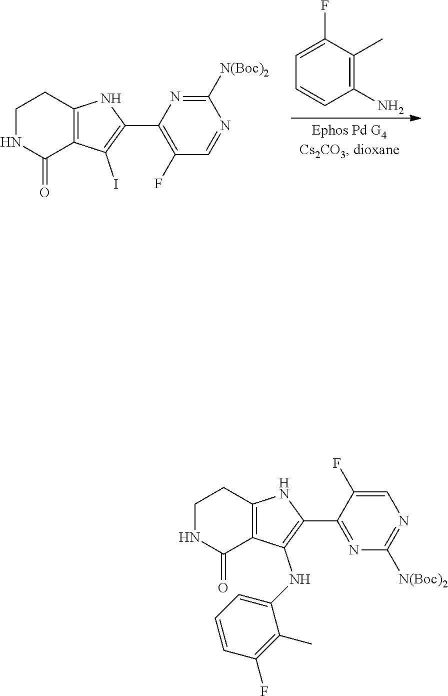

To a stirred mixture of tert-butyl N-(tert-butoxycarbonyl)-N-(5-fluoro-4-[3-iodo-4-oxo-1H,5H,6H,7H-pyrrolo[3,2-c]pyridin-2-yl]pyrimidin-2-yl)carbamate (200 mg, 0.35 mmol, 1.00 equiv) and 3-fluoro-2-methyl-aniline (65 mg, 0.52 mmol, 1.50 equiv) in dioxane (3 mL) were added Ephos Pd G4 (64 mg, 0.07 mmol, 0.20 equiv) and Cs₂CO₃ (227.3 mg, 0.70 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by Prep-TLC (CH₂Cl₂/MeOH 20:1) to affordtert-butyl N-(tert-butoxycarbonyl)-N-(5-fluoro-4-[3-[(3-fluoro-2-methylphenyl)amino]-4-oxo-1H,5H,6H,7H-pyrrolo[3,2-c]pyridin-2-yl]pyrimidin-2-yl)carbamate (95 mg, 47.73%) as a yellow oil.

LC-MS: M+H found: 571.0.

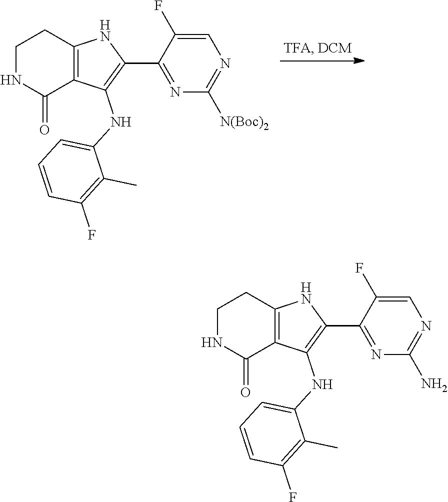

To a stirred mixture of tert-butyl N-(tert-butoxycarbonyl)-N-(5-fluoro-4-[3-[(3-fluoro-2-methylphenyl)amino]-4-oxo-1H,5H,6H,7H-pyrrolo[3,2-c]pyridin-2-yl]pyrimidin-2-yl)carbamate (95 mg, 1.00 equiv) in DCM (1.2 mL) and TFA (0.4 mL) was stirred for 1 h at room temperature under nitrogen atmosphere. The resulting mixture was concentrated under reduced pressure. The crude product was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 19% B to 30% B in 8 min, 30% B; Wave Length: 254/220 nm; RT1 (min): 7.13) to afford 2-(2-amino-5-fluoropyrimidin-4-yl)-3-[(3-fluoro-2-methoxyphenyl)amino]-1H,5H,6H,7H-pyrrolo[3,2-c]pyridin-4-one (13.7 mg, 23.67%)2-(2-amino-5-fluoropyrimidin-4-yl)-3-[(3-fluoro-2-methylphenyl)amino]-1H,5H,6H,7H-pyrrolo[3,2-c]pyridin-4-one (17.8 mg, 28.52%) as a orange solid.

LC-MS: M+H found: 371.0.

¹H NMR (400 MHz, DMSO-d6) δ 11.35 (s, 1H), 8.45 (s, 1H), 8.13 (d, J=3.7 Hz, 1H), 7.09 (d, J=2.9 Hz, 1H), 6.87 (q, J=7.8 Hz, 1H), 6.54 (t, J=8.8 Hz, 1H), 6.47 (d, J=8.2 Hz, 1H), 6.37 (s, 2H), 3.40 (d, J=2.7 Hz, 2H), 2.85 (t, J=6.7 Hz, 2H), 2.20 (d, J=1.8 Hz, 3H).

### Example 209. 3-[(3-fluoro-2-methylphenyl)amino]-2-[2-(methylamino)pyrimidin-4-yl]-1H,5H,6H,7H-pyrrolo[3,2-c]pyridin-4-one (compound 690)

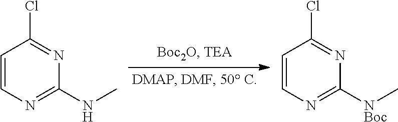

To a stirred mixture of 4-chloro-N-methylpyrimidin-2-amine (2.00 g, 13.93 mmol, 1.00 equiv) and Boc2O (7.60 g, 34.83 mmol, 2.5 equiv) in DMF (40.00 mL) were added TEA (3.52 g, 34.83 mmol, 2.50 equiv) and DMAP (0.51 g, 4.18 mmol, 0.3 equiv) in portions at room temperature under nitrogen atmosphere. The resulting mixture was stirred for 1 h at 50 degrees C. under nitrogen atmosphere. The resulting mixture was extracted with EtOAc (3×30 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EtOAc (5:1) to afford tert-butyl N-(4-chloropyrimidin-2-yl)-N-methylcarbamate (3 g, 88.37%) as a yellow oil.

LC-MS: M+H found: 244.

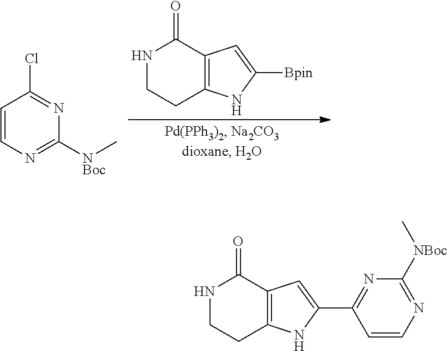

To a stirred mixture of tert-butyl N-(4-chloropyrimidin-2-yl)-N-methylcarbamate (2.80 g, 11.49 mmol, 1.00 equiv) and 2-(4,4,5,5-tetramethyl-1,3,2-dioxaborolan-2-yl)-1H,5H,6H,7H-pyrrolo[3,2-c]pyridin-4-one (6.02 g, 22.98 mmol, 2.00 equiv) in dioxane (100.00 mL) and H₂O (20.00 mL) were added Pd(PPh₃)₄ (1.33 g, 1.15 mmol, 0.1 equiv) and Na2CO3 (2.44 g, 23.0 mmol, 2 equiv) in portions at room temperature under argon atmosphere. The resulting mixture was stirred for overnight at 60 degrees C. under argon atmosphere. The resulting mixture was extracted with EtOAc (3×50 mL). The combined organic layers were washed with brine (lx20 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-methyl-N-(4-[4-oxo-1H,5H,6H,7H-pyrrolo[3,2-c]pyridin-2-yl]pyrimidin-2-yl)carbamate (4.5 g, 96.95%) as a light yellow solid.

LC-MS: (M+H)⁺ found: 344.

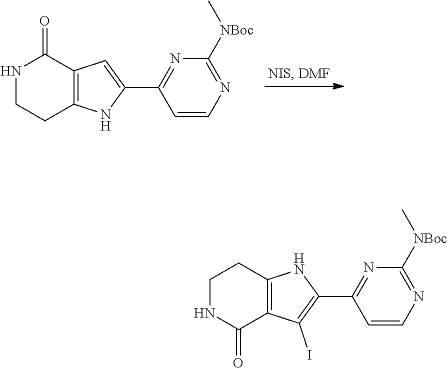 [SMILES: CN(C(=O)OC(C)(C)C)c1nccc(-c2cc3c([nH]2)CCNC3=O)n1 ; CN(C(=O)OC(C)(C)C)c1nccc(-c2[nH]c3c(c2I)C(=O)NCC3)n1]

A mixture of tert-butyl N-methyl-N-(4-[4-oxo-1H,5H,6H,7H-pyrrolo[3,2-c]pyridin-2-yl]pyrimidin-2-yl)carbamate (2.00 g, 5.82 mmol, 1.00 equiv) and NIS (1.57 g, 6.98 mmol, 1.20 equiv) in DMF (20.00 mL) was stirred for 2 h at room temperature under nitrogen atmosphere. The reaction was quenched by the addition of sat. Na2SO3 (aq.) (10 mL) at 0 degrees C. The resulting mixture was diluted with water (IOmL). The precipitated solids were collected by filtration and washed with water (1×5 mL) to afford tert-butyl N-(4-[3-iodo-4-oxo-1H,5H,6H,7H-pyrrolo[3,2-c]pyridin-2-yl]pyrimidin-2-yl)-N-methylcarbamate (2 g, 73.17%) as a light yellow solid.

LC-MS: (M+H)⁺ found: 470.

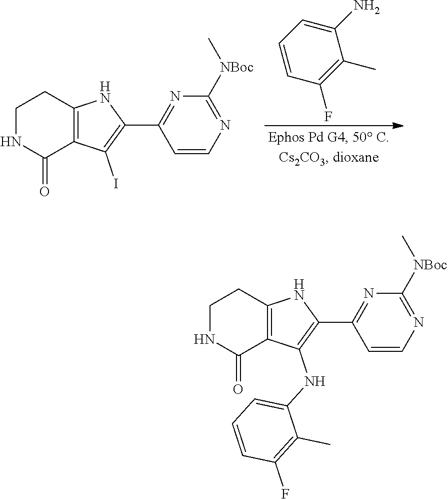

To a stirred mixture of tert-butyl N-(4-[3-iodo-4-oxo-1H,5H,6H,7H-pyrrolo[3,2-c]pyridin-2-yl]pyrimidin-2-yl)-N-methylcarbamate (300 mg, 0.64 mmol, 1.00 equiv) and 3-fluoro-2-methyl-aniline (240 mg, 1.92 mmol, 3 equiv) in dioxane (6.00 mL) were added Ephos Pd G4 (117 mg, 0.13 mmol, 0.2 equiv) and Cs₂CO₃ (417 mg, 1.28 mmol, 2 equiv) in portions at 50 degrees C. under argon atmosphere. The resulting mixture was stirred for overnight at 50 degrees C. under nitrogen atmosphere. The resulting mixture was filtered, the filter cake was washed with EtOAc (2×10 mL). The filtrate was concentrated under reduced pressure. The residue was purified by Prep-TLC (CH₂Cl₂/MeOH 10:1) to afford tert-butyl N-(4-[3-[(3-fluoro-2-methylphenyl)amino]-4-oxo-1H,5H,6H,7H-pyrrolo[3,2-c]pyridin-2-yl]pyrimidin-2-yl)-N-methylcarbamate (150 mg, 50.30%) as a yellow solid.

LC-MS: (M+H)⁺ found: 467.

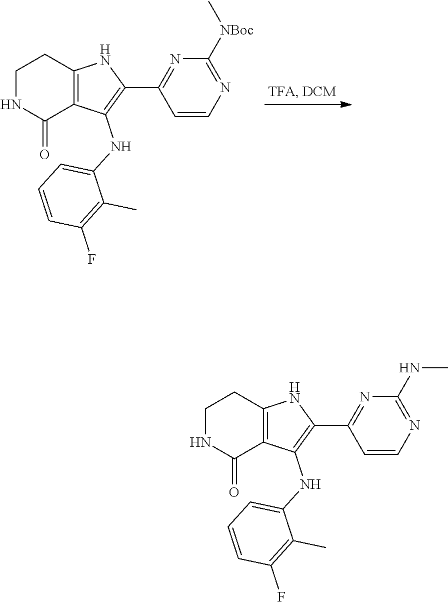 [SMILES: Cc1c(F)cccc1Nc1c(-c2ccnc(N(C)C(=O)OC(C)(C)C)n2)[nH]c2c1C(=O)NCC2 ; CNc1nccc(-c2[nH]c3c(c2Nc2cccc(F)c2C)C(=O)NCC3)n1]

A mixture of tert-butyl N-(4-[3-[(3-fluoro-2-methylphenyl)amino]-4-oxo-1H,5H,6H,7H-pyrrolo[3,2-c]pyridin-2-yl]pyrimidin-2-yl)-N-methylcarbamate (140 mg, 0.30 mmol, 1.00 equiv) in TFA (2.00 mL) and DCM (2.00 mL) was stirred for 3 h at room temperature under nitrogen atmosphere. The resulting mixture was concentrated under reduced pressure. The crude product (140 mg) was purified by Prep-HPLC with the following conditions (Column: YMC-Actus Triart C18, 30*150 mm, 5 μm; Mobile Phase A: Water (1OMMOL/L NH4HCO3+0. 1% NH3·H2O), Mobile Phase B: ACN; Flow rate: 60 mUmin, Gradient: 27% B to 57% B in 8 min, Wave Length: 254/220 nm; RT1 (min): 7.68) to afford 3-[(3-fluoro-2-methylphenyl)amino]-2-[2-(methylamino)pyrimidin-4-yl]-1H,5H,6H,7H-pyrrolo[3,2-c]pyridin-4-one (53.7 mg, 47.81%) as a light yellow solid.

LC-MS: (M+H)⁺ found: 367.05.

¹H NMR (400 MHz, DMSO-d6) δ 11.54 (s, 1H), 8.03 (d, J=5.3 Hz, 1H), 7.60 (s, 1H), 7.10 (d, J=2.7 Hz, 1H), 6.88 (m, J=7.8 Hz, 1H), 6.69 (s, 1H), 6.54 (d, J=8.3 Hz, 1H), 6.42 (d, J=5.0 Hz, 1H), 6.29 (d, J=8.2 Hz, 1H), 3.40 (d, J=2.6 Hz, 2H), 2.85 (d, J=5.8 Hz, 5H), 2.20 (d, J=1.7 Hz, 3H).

### Example 210. 3-[(3-fluoro-2-methoxyphenyl)amino]-2-[2-(methylamino)pyrimidin-4-yl]-1H,5H,6H,7H-pyrrolo[3,2-c]pyridin-4-one (compound 691)

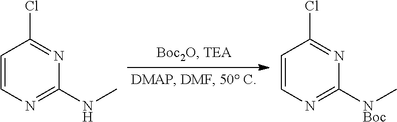

To a stirred mixture of 4-chloro-N-methylpyrimidin-2-amine (2.00 g, 13.93 mmol, 1.00 equiv) and Boc20 (7.60 g, 34.83 mmol, 2.5 equiv) in DMF (40.00 mL) were added TEA (3.52 g, 34.83 mmol, 2.50 equiv) and DMAP (0.51 g, 4.18 mmol, 0.3 equiv) in portions at room temperature under nitrogen atmosphere. The resulting mixture was stirred for 1 h at 50 degrees C. under nitrogen atmosphere. The resulting mixture was extracted with EtOAc (3×30 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EtOAc (5:1) to afford tert-butyl N-(4-chloropyrimidin-2-yl)-N-methylcarbamate (3 g, 88.37%) as a yellow oil.

LC-MS: M+H found: 244.

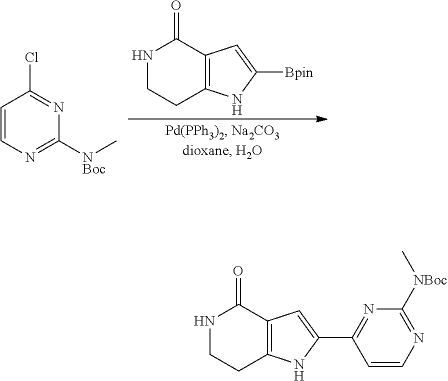

To a stirred mixture of tert-butyl N-(4-chloropyrimidin-2-yl)-N-methylcarbamate (2.80 g, 11.490 mmol, 1.00 equiv) and 2-(4,4,5,5-tetramethyl-1,3,2-dioxaborolan-2-yl)-1H,5H,6H,7H-pyrrolo[3,2-c]pyridin-4-one (6.02 g, 22.98 mmol, 2.00 equiv) in dioxane (100.00 mL) and H₂O (20.00 mL) were added Pd(PPh₃)₄ (1.33 g, 1.15 mmol, 0.1 equiv) and Na₂CO₃ (2.44 g, 22.98 mmol, 2 equiv) in portions at room temperature under argon atmosphere. The resulting mixture was stirred for overnight at 60 degrees C. under argon atmosphere. The resulting mixture was extracted with EtOAc (3×50 mL). The combined organic layers were washed with brine (1×20 mL), dried over anhydrous Na₂SO₄. After filtration, the filtrate was concentrated under reduced pressure. The residue was purified by silica gel column chromatography, eluted with CH₂Cl₂/MeOH (10:1) to afford tert-butyl N-methyl-N-(4-[4-oxo-1H,5H,6H,7H-pyrrolo[3,2-c]pyridin-2-yl]pyrimidin-2-yl)carbamate (4.5 g, 96.95%) as a light yellow solid.

LC-MS: (M+H)⁺ found: 344.

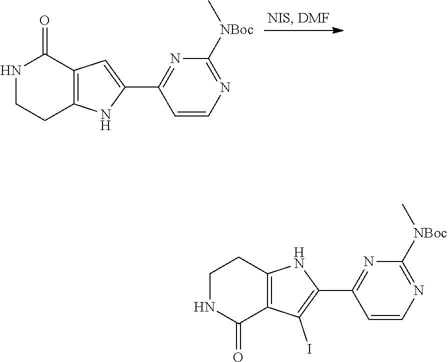 [SMILES: CN(C(=O)OC(C)(C)C)c1nccc(-c2cc3c([nH]2)CCNC3=O)n1 ; CN(C(=O)OC(C)(C)C)c1nccc(-c2[nH]c3c(c2I)C(=O)NCC3)n1]

A mixture of tert-butyl N-methyl-N-(4-[4-oxo-1H,5H,6H,7H-pyrrolo[3,2-c]pyridin-2-yl]pyrimidin-2-yl)carbamate (2.00 g, 5.82 mmol, 1.00 equiv) and NIS (1.57 g, 6.98 mmol, 1.20 equiv) in DMF (20.00 mL) was stirred for 2 h at room temperature under nitrogen atmosphere. The reaction was quenched by the addition of sat. Na2SO3 (aq.) (10 mL) at 0 degrees C. The resulting mixture was diluted with water (IOmL). The precipitated solids were collected by filtration and washed with water (1×5 mL) to afford tert-butyl N-(4-[3-iodo-4-oxo-1H,5H,6H,7H-pyrrolo[3,2-c]pyridin-2-yl]pyrimidin-2-yl)-N-methylcarbamate (2 g, 73.17%) as a light yellow solid.

LC-MS: (M+H)⁺ found: 470.

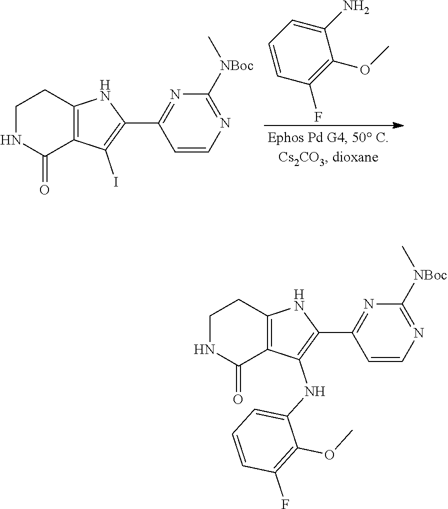

To a stirred mixture of tert-butyl N-(4-[3-iodo-4-oxo-1H,5H,6H,7H-pyrrolo[3,2-c]pyridin-2-yl]pyrimidin-2-yl)-N-methylcarbamate (300 mg, 0.64 mmol, 1.00 equiv) and 3-fluoro-2-methoxyaniline (271 mg, 1.98 mmol, 3.00 equiv) in dioxane (6.00 mL) were added Ephos Pd G4 (117 mg, 0.13 mmol, 0.20 equiv) and Cs₂CO₃ (417 mg, 1.28 mmol, 2.00 equiv) in portions at 50 degrees C. under argon atmosphere. The resulting mixture was stirred for overnight at 50 degrees C. under argon atmosphere. The resulting mixture was filtered, the filter cake was washed with EtOAc (2×10 mL). The filtrate was concentrated under reduced pressure. The residue was purified by Prep-TLC (CH₂Cl₂/MeOH 10:1) to afford tert-butyl N-(4-[3-[(3-fluoro-2-methoxyphenyl)amino]-4-oxo-1H,5H,6H,7H-pyrrolo[3,2-c]pyridin-2-yl]pyrimidin-2-yl)-N-methylcarbamate (170 mg, 55.11%) as a yellow solid.

LC-MS: (M+H)⁺ found: 483.

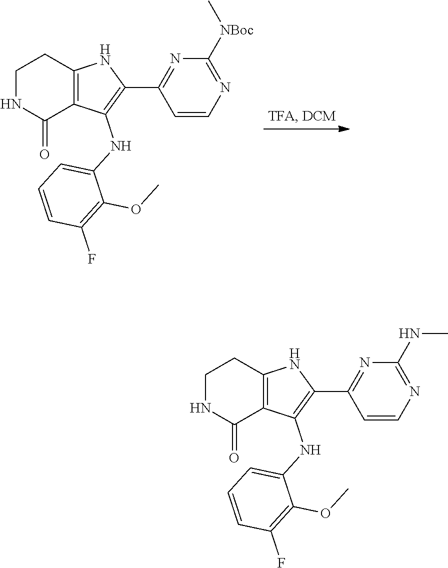 [SMILES: COc1c(F)cccc1Nc1c(-c2ccnc(N(C)C(=O)OC(C)(C)C)n2)[nH]c2c1C(=O)NCC2 ; CNc1nccc(-c2[nH]c3c(c2Nc2cccc(F)c2OC)C(=O)NCC3)n1]

A mixture of tert-butyl N-(4-[3-[(3-fluoro-2-methoxyphenyl)amino]-4-oxo-1H,5H,6H,7H-pyrrolo[3,2-c]pyridin-2-yl]pyrimidin-2-yl)-N-methylcarbamate (160 mg, 0.33 mmol, 1.00 equiv) in TFA (2.00 mL) and DCM (2.00 mL) was stirred for 2 h at room temperature under nitrogen atmosphere. The resulting mixture was concentrated under reduced pressure. The crude product (170 mg) was purified by Prep-HPLC with the following conditions (Column: XBridge Shield RP18 OBD Column, 30*150 mm, 5plm; Mobile Phase A: Water (1OMMOL/L NH4HCO3+0.1% NH3·H2O), Mobile Phase B: ACN; Flow rate: 60 m/min; Gradient: 27% B to40% B in 8 min; Wave Length: 254; 220 nm; RT1 (min): 6.35) to afford 3-[(3-fluoro-2-methoxyphenyl)amino]-2-[2-(methylamino)pyrimidin-4-yl]-1H,5H,6H,7H-pyrrolo[3,2-c]pyridin-4-one (50.1 mg, 39.35%) as a yellow solid.

LC-MS: (M+H)⁺ found: 383.15.

¹H NMR (400 MHz, DMSO-d6) δ 11.57 (s, 1H), 8.06 (d, J=5.3 Hz, 1H), 7.89 (s, 1H), 7.13 (t, J=2.7 Hz, 1H), 6.76 (m, J=6.0 Hz, 1H), 6.68 (s, 1H), 6.57 (m, J=1.5 Hz, 1H), 6.47 (d, J=5.3 Hz, 1H), 6.24 (m, J=1.3 Hz, 1H), 3.90 (s, 3H), 3.40 (d, J=2.6 Hz, 2H), 2.86 (d, J=5.7 Hz, 5H).

### Example 211. 2-(2-amino-3-fluoropyridin-4-yl)-3-[(3-fluoro-2-methylphenyl)amino]-1H,5H,6H,7H-pyrrolo[3,2-c]pyridin-4-one (compound 692)

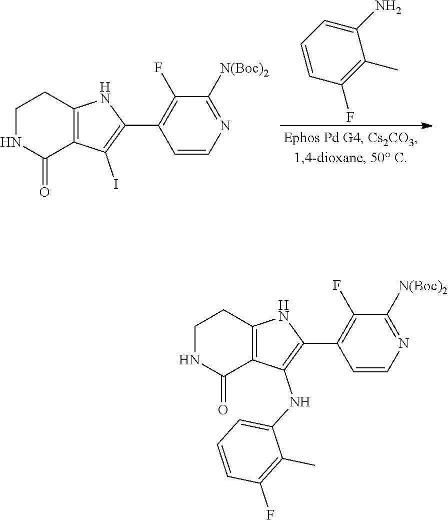

To a stirred mixture of tert-butyl N-(tert-butoxycarbonyl)-N-(3-fluoro-4-[3-iodo-4-oxo-1H,5H,6H,7H-pyrrolo[3,2-c]pyridin-2-yl]pyridin-2-yl)carbamate (270 mg, 0.472 mmol, 1.00 equiv) and 3-fluoro-2-methyl-aniline (177 mg, 1.42 mmol, 3.00 equiv) in 1,4-dioxane (5.00 mL) were added EPhos Pd G4 (87 mg, 0.09 mmol, 0.20 equiv) and Cs₂CO₃ (308 mg, 0.94 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl2/MeOH (20:1) to afford tert-butyl N-(tert-butoxycarbonyl)-N-(3-fluoro-4-[3-[(3-fluoro-2-methylphenyl)amino]-4-oxo-1H,5H,6H,7H-pyrrolo[3,2-c]pyridin-2-yl]pyridin-2-yl)carbamate (250 mg, 92.94%) as a yellow solid.

LC-MS: (M+H found: 570.35.

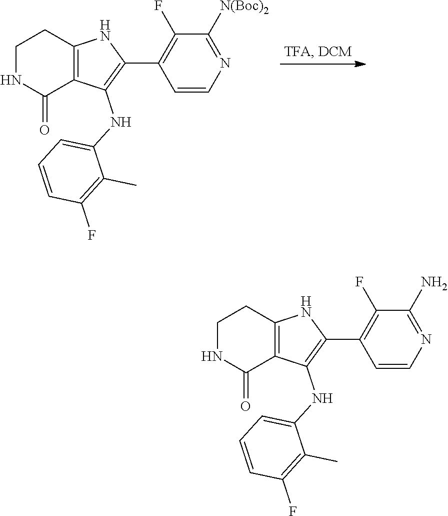

To a stirred mixture of tert-butyl N-(tert-butoxycarbonyl)-N-(3-fluoro-4-[3-[(3-fluoro-2-methylphenyl)amino]-4-oxo-1H,5H,6H,7H-pyrrolo[3,2-c]pyridin-2-yl]pyridin-2-yl)carbamate (220 mg, 0.39 mmol, 1.00 equiv) in DCM (2 mL) was added TFA (1 mL) at room temperature under nitrogen atmosphere. The resulting mixture was stirred for 1 h and concentrated under reduced pressure. The crude product (150 mg) was purified by Prep-HPLC with the following conditions (Column: XBridge Prep OBD C18 Column, 30*150 mm, 5 μm; Mobile Phase A: Water (1OMMOL/L NH4HCO3), Mobile Phase B: ACN; Flow rate: 60 mL/min; Gradient: 17% B to 47% B in 7 min; Wave Length: 254 nm; RT1 (min): 6.5) to afford 2-(2-amino-3-fluoropyridin-4-yl)-3-[(3-fluoro-2-methylphenyl)amino]-1H,5H,6H,7H-pyrrolo[3,2-c]pyridin-4-one (69.4 mg, 48.21%) as a white solid.

LC-MS: (M+H)⁺ found: 570.35.

¹H NMR (300 MHz, DMSO-d6): δ 11.37 (s, 1H), 7.53 (d, J=5.4 Hz, 1H), 7.17 (d, J=20.1 Hz, 2H), 6.81-6.73 (m, 1H), 6.55 (t, J=5.1 Hz, 1H), 6.43 (t, J1=9.0 Hz, 1H), 6.10 (d, J=9.6 Hz, 3H), 3.42-3.38 (m, 2H), 2.83 (t, J=6.9 Hz, 2H), 2.15 (s, 3H).

### Example 212. 2-amino-4-[3-[(3-chloro-2-methoxyphenyl)amino]-4-oxo-1H,5H,6H,7H-pyrrolo[3,2-c]pyridin-2-yl]pyrimidine-5-carbonitrile (compound 693)

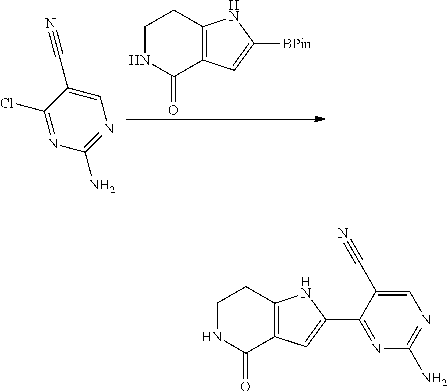

To a stirred solution of 2-amino-4-chloropyrimidine-5-carbonitrile (500 mg, 3.23 mmol, 1.00 equiv) and 2-(4,4,5,5-tetramethyl-1,3,2-dioxaborolan-2-yl)-1H,5H,6H,7H-pyrrolo[3,2-c]pyridin-4-one (1.69 g, 6.47 mmol, 2.00 equiv) in 1,4-dioxane (10.00 mL) and H₂O (2.00 mL) were added K2CO3 (894 mg, 6.47 mmol, 2.00 equiv) and Pd(PPh₃)₄ (747 mg, 0.64 mmol, 0.20 equiv) dropwise at room temperature under nitrogen atmosphere. The resulting mixture was stirred for overnight at 80° C. under nitrogen atmosphere. Desired product could be detected by LCMS. The precipitated solids were collected by filtration and washed with CH₂Cl₂/MeOH=10/1 (3×10 mL). The resulting solid was dried under vacuum to give 2-amino-4-(4-oxo-4,5,6,7-tetrahydro-1H-pyrrolo[3,2-c]pyridin-2-yl)pyrimidine-5-carbonitrile (600 mg, crude) as a yellow solid.

LC-MS: (M+H)⁺ found: 255.20.

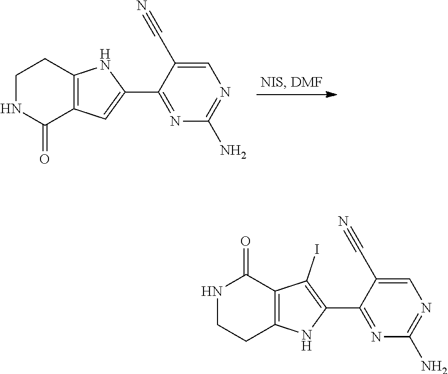 [SMILES: N#Cc1cnc(N)nc1-c1cc2c([nH]1)CCNC2=O ; N#Cc1cnc(N)nc1-c1[nH]c2c(c1I)C(=O)NCC2]

To a stirred solution of 2-amino-4-{4-oxo-1H,5H,6H,7H-pyrrolo[3,2-c]pyridin-2-yl}pyrimidine-5-carbonitrile (500 mg, 1.96 mmol, 1.00 equiv) in DMF (5.00 mL) was added NIS (663 mg, 2.95 mmol, 1.5 equiv) dropwise at 0° C. under argon atmosphere. The resulting mixture was stirred for 2 h at 0° C. under nitrogen atmosphere. The reaction was quenched by the addition of sat. Na2SO3 (aq.) (2 mL) at 0° C. The resulting mixture was filtered, the filter cake was washed with ethyl acetate (3×10 mL). The resulting solid was dried under vacuum to give 2-amino-4-{3-iodo-4-oxo-1H,5H,6H,7H-pyrrolo[3,2-c]pyridin-2-yl}pyrimidine-5-carbonitrile (230 mg, crude) as a yellow solid.

LC-MS: (M+H)⁺ found: 380.10.

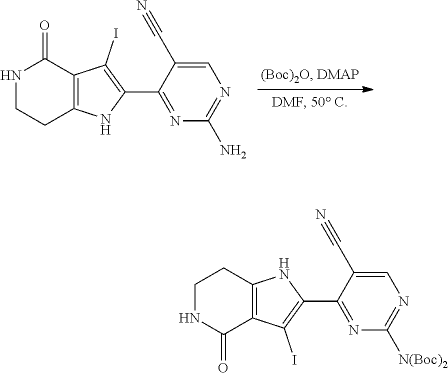

To a stirred solution of 2-amino-4-{3-iodo-4-oxo-1H,5H,6H,7H-pyrrolo[3,2-c]pyridin-2-yl}pyrimidine-5-carbonitrile (220 mg, 0.57 mmol, 1.00 equiv) in DMF (10 mL) was added (Boc)₂O (315 mg, 1.45 mmol, 2.5 equiv) dropwise at room temperature under argon atmosphere. The resulting mixture was stirred for overnight at 50° C. under argon atmosphere. The resulting mixture was extracted with CH₂Cl₂/MeOH (10/1) (3×30 mL). The combined organic layers were washed with saturated salt water (3×30 mL), dried over anhydrous Na₂SO₄. After filtration, the filtrate was concentrated under reduced pressure. The residue was purified by Prep-TLC (CH₂Cl₂/MeOH 20:1) to afford tert-butyl N-(tert-butoxycarbonyl)-N-(5-cyano-4-{3-iodo-4-oxo-1H,5H,6H,7H-pyrrolo[3,2-c]pyridin-2-yl}pyrimidin-2-yl)carbamate (220 mg, 65.50%) as a light yellow oil.

LC-MS: (M+H)⁺ found: 581.00.

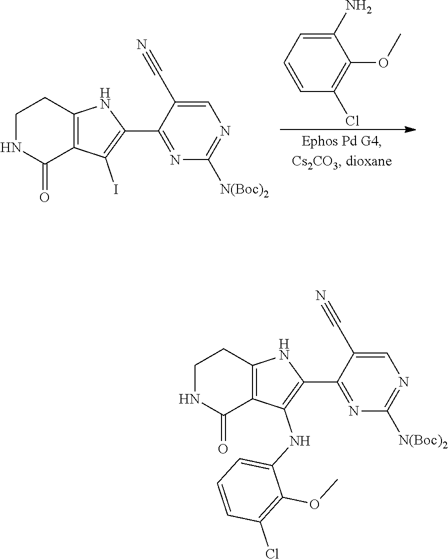

To a stirred solution of tert-butyl N-(tert-butoxycarbonyl)-N-(5-cyano-4-(3-iodo-4-oxo-1H,5H,6H,7H-pyrrolo[3,2-c]pyridin-2-yl pyrimidin-2-yl)carbamate (200 mg, 0.34 mmol, 1.00 equiv) in DMF (2.00 mL) was added Cs₂CO₃ (336 mg, 1.03 mmol, 3.00 equiv), Cs₂CO₃ (336 mg, 1.03 mmol, 3.00 equiv) dropwise at room temperature under argon atmosphere. The resulting mixture was stirred for 2 h at 50° C. under argon atmosphere. The resulting mixture was extracted with CH₂C2I/MeOH (10:1) (3×10 mL). The combined organic layers were washed with saturated salt water (1×20 mL), dried over anhydrous Na₂SO₄. After filtration, the filtrate was concentrated under reduced pressure. The residue was purified by silica gel column chromatography, eluted with CH₂Cl2/MeOH (20:1) to afford tert-butyl N-(tert-butoxycarbonyl)-N-(4-{3-[(3-chloro-2-methoxyphenyl)amino]-4-oxo-1H,5H,6H,7H-pyrrolo[3,2-c]pyridin-2-yl}-5-cyanopyrimidin-2-yl)carbamate (90 mg, 42.81%) as a light yellow oil.

LC-MS: (M+H)⁺ found: 610.10.

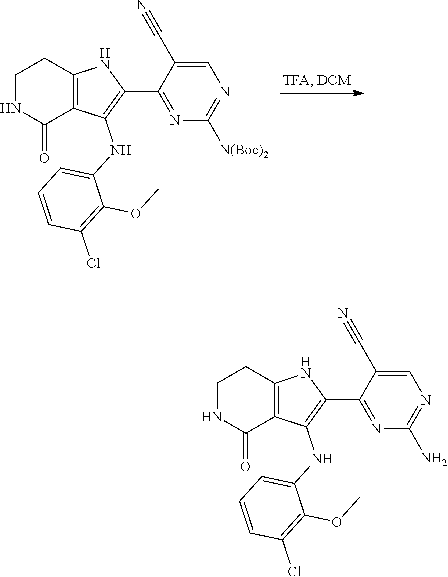

To a stirred solution of tert-butyl N-(tert-butoxycarbonyl)-N-(4-[3-[(3-chloro-2-methoxyphenyl)amino]-4-oxo-1H,5H,6H,7H-pyrrolo[3,2-c]pyridin-2-yl]-5-cyanopyrimidin-2-yl)carbamate (90 mg, 0.14 mmol, 1.00 equiv) in DCM (2.00 mL) was added TFA (1.00 mL) at 0° C. under nitrogen atmosphere. The resulting mixture was stirred for 1 h at room temperature under nitrogen atmosphere. The resulting mixture was concentrated under reduced pressure. The crude product was purified by Prep-HPLC with the following conditions (Column: XBridge Prep OBD C18 Column, 19*250 mm, 5 μm; Mobile Phase A: Water (10 mmol/L NH4HCO3), Mobile Phase B: MeOH-Preparative; Flow rate: 25 mL/min; Gradient: 25% B to 38% B in 10 min, 38% B; Wave Length: 254 nm; RT1 (min): 8.67) to afford 2-amino-4-[3-[(3-chloro-2-methoxyphenyl)amino]-4-oxo-1H,5H,6H,7H-pyrrolo[3,2-c]pyridin-2-yl]pyrimidine-5-carbonitrile (7.2 mg, 11.31%) as a yellow solid.

LC-MS: (M+H)⁺ found: 410.00.

¹H NMR (400 MHz, CD₃OD-d₄) δ 9.17 (s, 9H), 7.65-7.56 (m, 1H), 7.31-7.19 (m, 2H), 3.72 (s, 3H), 3.47-3.38 (m, 2H), 2.98-2.88 (m, 2H).

### Example 213. 2-[6-(tert-butylamino)-1,5-naphthyridin-4-yl]-3-[(3-fluoro-2-methoxyphenyl)amino]-1H,5H,6H,7H-pyrrolo[3,2-c]pyridin-4-one (compound 694)

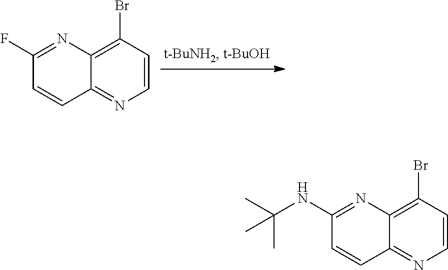 [SMILES: Fc1ccc2nccc(Br)c2n1 ; CC(C)(C)Nc1ccc2nccc(Br)c2n1]

To a stirred solution of 8-bromo-2-fluoro-1,5-naphthyridine (500.00 mg, 2.202 mmol, 1.00 equiv) and t-BuOH (4897.13 mg, 66.069 mmol, 30.00 equiv) in tert-butylamine borane (5.00 mL) in portions at degrees 100 C under nitrogen atmosphere. The resulting solid was dried under vacuum to afford 8-bromo-N-tert-butyl-1,5-naphthyridin-2-amine (650 mg, 94.81%) as a white solid.

LC-MS: M+H found: 280.0.

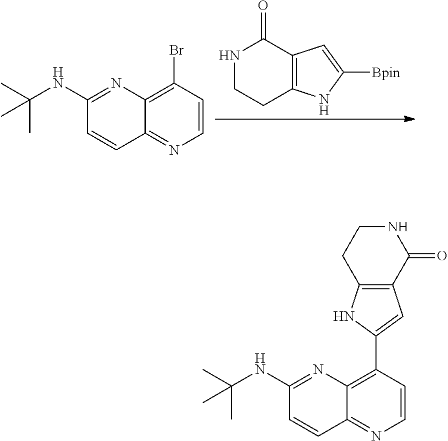

To a stirred solution 8-bromo-N-tert-butyl-1,5-naphthyridin-2-amine (600.00 mg, 2.142 mmol, 1.00 equiv) and TEA (541.76 mg, 5.354 mmol, 2.50 equiv) in DME (14.30 mL) and H2O (0.70 mL) were added Pd(dppf)Cl2 (156.70 mg, 0.214 mmol, 0.10 equiv) and 2-(4,4,5,5-tetramethyl-1,3,2-dioxaborolan-2-yl)-1H,5H,6H,7H-pyrrolo[3,2-c] pyridin-4-one (842.02 mg, 3.212 mmol, 1.50 equiv) in portions at degrees 80 C under nitrogen atmosphere. The residue was purified by Prep-TLC (CH₂Cl₂/MeOH 10:1) to afford 2-[6-(tert-butylamino)-1,5-naphthyridin-4-yl]-1H,5H,6H,7H-pyrrolo[3,2-c] pyridin-4-one (230 mg, 27.54%) as a yellow solid.

LC-MS: M+H found 336.0.

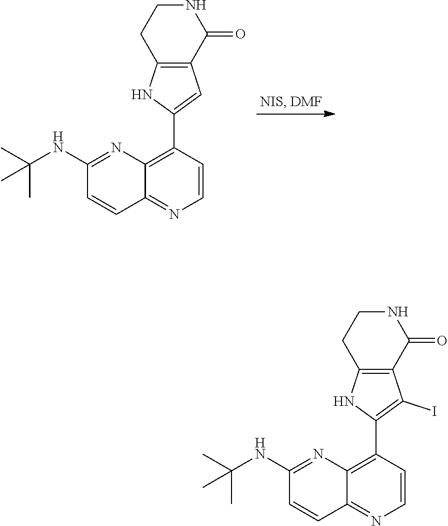 [SMILES: CC(C)(C)Nc1ccc2nccc(-c3cc4c([nH]3)CCNC4=O)c2n1 ; CC(C)(C)Nc1ccc2nccc(-c3[nH]c4c(c3I)C(=O)NCC4)c2n1]

A solution of 2-[6-(isopropylamino)-1,5-naphthyridin-4-yl]-1H,5H,6H,7H-pyrrolo[3,2-c]pyridin-4-one (120.00 mg, 0.373 mmol, 1.00 equiv) and NIS (168.01 mg, 0.747 mmol, 2.00 equiv) in DMF (2.50 mL) was stirred for 2 h at room temperature under nitrogen atmosphere. The residue was purified by Prep-TLC (CH₂Cl2/MeOH 10:1) to afford 2-[6-(tert-butylamino)-1,5-naphthyridin-4-yl]-3-iodo-1H,5H,6H,7H-pyrrolo[3,2-c] pyridin-4-one (60 mg, 37.62%) as a brown solid.

LC-MS: M+H found: 462.0.

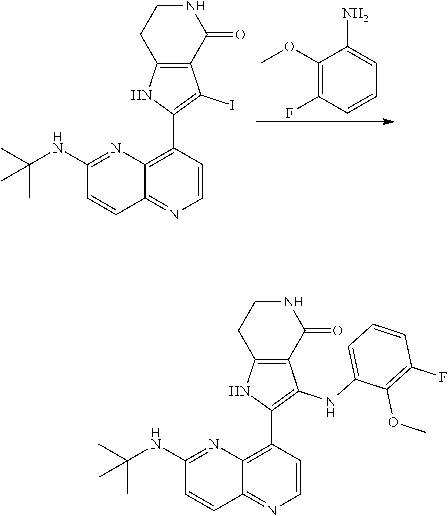

To a stirred solution 2-[6-(tert-butylamino)-1,5-naphthyridin-4-yl]-3-iodo-1H,5H,6H,7H-pyrrolo[3,2-c] pyridine-4-one (50.00 mg, 0.108 mmol, 1.00 equiv) and Cs2CO3 (70.63 mg, 0.217 mmol, 2.00 equiv) in dioxane (1.00 mL) were added EPhos Pd G4 (19.91 mg, 0.022 mmol, 0.20 equiv) and 3-fluoro-2-methoxyaniline (19.89 mg, 0.141 mmol, 1.30 equiv) in portions at degrees 50 C under nitrogen atmosphere. The resulting mixture was stirred for overnight at degrees 50 C under nitrogen atmosphere. The residue was purified by Prep-TLC (CH2Cl2/MeOH 10:1) to afford 2-[6-(tert-butylamino)-1,5-naphthyridin-4-yl]-3-[(3-fluoro-2-methoxyphenyl) amino]-1H,5H,6H,7H-pyrrolo[3,2-c] pyridin-4-one (50 mg, 68.05%) as a yellow solid. The crude product (mg) was purified by Prep-HPLC with the following conditions (Column: XBridge Prep OBD C18 Column, 30*150 mm, 5 μm; Mobile Phase A: Water (1OMMOL/L NH4HCO3), Mobile Phase B: ACN; Flow rate: 60 mUmin; Gradient: 20% B to 45% B in 7 min; Wave Length: 254 nm; RT1 (min): 6.5) to afford 2-[6-(tert-butylamino)-1,5-naphthyridin-4-yl]-3-[(3-fluoro-2-methoxyphenyl)amino]-1H,5H,6H,7H-pyrrolo[3,2-c]pyridin-4-one (50 mg, 68.05%) as a yellow solid.

LC-MS: M+H found: 475.1.

¹H NMR (400 MHz, DMSO-d6) δ 13.39 (s, 1H), 8.40 (d, J=5.9 Hz, 1H), 8.15-7.75 (m, 3H), 7.39 (dd, J=19.5, 12.4 Hz, 3H), 6.81-6.51 (m, 2H), 6.09 (d, J=8.0 Hz, 1H), 4.00 (s, 3H), 3.49 (d, J=8.4 Hz, 2H), 3.01 (t, J=6.8 Hz, 2H), 1.58 (s, 9H).

### Example 214. 3-[(3-fluoro-2-methoxyphenyl)amino]-2-[6-[(1-methylcyclopropyl)amino]-1,5-naphthyridin-4-yl]-1H,5H,6H,7H-pyrrolo[3,2-c]pyridin-4-one; formic acid (compound 695)

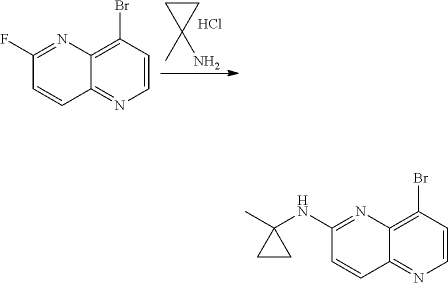

To a stirred solution of 8-bromo-2-fluoro-1,5-naphthyridine (400 mg, 1.762 mmol, 1.00 equiv) and 1-methylcyclopropan-1-amine (626.53 mg, 8.810 mmol, 5 equiv) in t-BuOH (5 mL) were added K₂CO₃ (1217.48 mg, 8.810 mmol, 5 equiv) at 0 degrees C. under nitrogen atmosphere. The resulting mixture was stirred for 5 h at room temperature under nitrogen atmosphere. Desired product could be detected by LCMS. The resulting mixture was diluted with water (15 mL). The aqueous layer was extracted with EtOAc (3×15 mL). The resulting mixture was concentrated under reduced pressure. This resulted in 8-bromo-N-(1-methylcyclopropyl)-1,5-naphthyridin-2-amine (500 mg, 102.03%) as a white solid.

LC-MS: [M−H]⁻ found: 278.05.

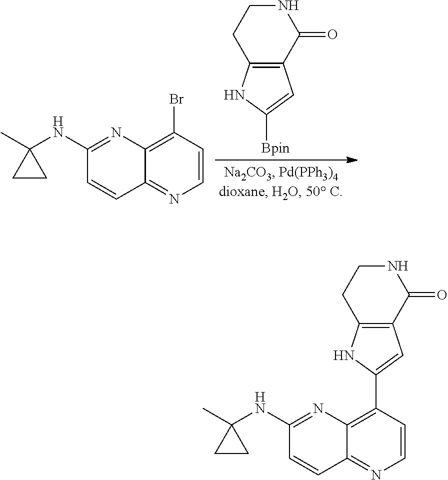

To a solution of 8-bromo-N-(1-methylcyclopropyl)-1,5-naphthyridin-2-amine (300 mg, 1.079 mmol, 1.00 equiv) and 2-(4,4,5,5-tetramethyl-1,3,2-dioxaborolan-2-yl)-1H,5H,6H,7H-pyrrolo[3,2-c]pyridin-4-one (424.06 mg, 1.619 mmol, 1.5 equiv) in dioxane (3 mL) and water (0.6 mL) were added Na2CO3 (228.63 mg, 2.158 mmol, 2 equiv) and Pd(PPh₃)₄ (124.63 mg, 0.108 mmol, 0.1 equiv). After stirring for overnight at 50 degrees C. under a nitrogen atmosphere, the resulting mixture was concentrated under reduced pressure. The residue was purified by silica gel column chromatography, eluted with CH2Cl2/MeOH (15:1) to afford 2-[6-[(1-methylcyclopropyl)amino]-1,5-naphthyridin-4-yl]-1H,5H,6H,7H-pyrrolo[3,2-c]pyridin-4-one (420 mg, 116.80%) as a light brown solid.

LC-MS: [M−H]⁻ found: 334.00.

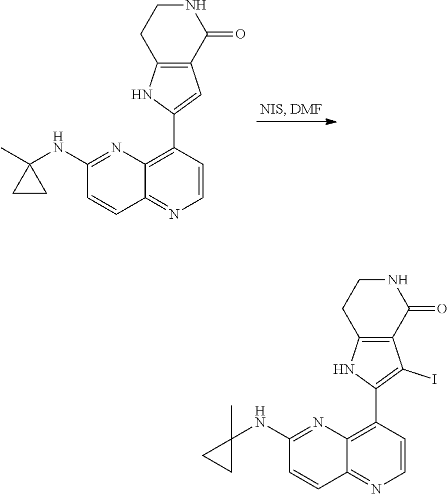 [SMILES: CC1(Nc2ccc3nccc(-c4cc5c([nH]4)CCNC5=O)c3n2)CC1 ; CC1(Nc2ccc3nccc(-c4[nH]c5c(c4I)C(=O)NCC5)c3n2)CC1]

A solution of 2-{6-[(1-methylcyclopropyl)amino]-1,5-naphthyridin-4-yl}-1H,5H,6H,7H-pyrrolo[3,2-c]pyridin-4-one (300 mg, 0.900 mmol, 1.00 equiv) and nis (222.69 mg, 0.990 mmol, 1.1 equiv) in DMF (3 mL) was stirred for overnight at room temperature under nitrogen atmosphere. Desired product could be detected by LCMS. The resulting solution was purified by reverse phase flash with the following conditions (10 mmol/L NH4HCO3, 40% ACN to 65% ACN in 20 min) to afford 3-iodo-2-{6-[(1-methylcyclopropyl)amino]-1,5-naphthyridin-4-yl}-1H,5H,6H,7H-pyrrolo[3,2-c]pyridin-4-one (350 mg, 84.69%) as a light brown solid.

LC-MS: [M−H]⁻ found: 459.95.

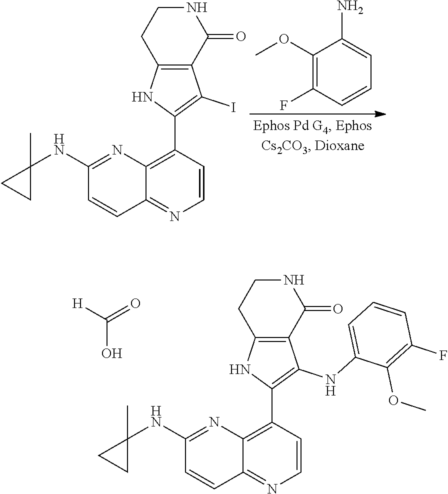

A mixture of 3-iodo-2-[6-[(1-methylcyclopropyl)amino]-1,5-naphthyridin-4-yl]-1H,5H,6H,7H-pyrrolo[3,2-c]pyridin-4-one (110.00 mg, 0.239 mmol, 1.00 equiv), 3-fluoro-2-methoxyaniline (101.41 mg, 0.718 mmol, 3.00 equiv), Ephos Pd G4 (44.00 mg, 0.048 mmol, 0.20 equiv), Ephos (25.62 mg, 0.048 mmol, 0.20 equiv) and Cs₂CO₃ (156.07 mg, 0.479 mmol, 2.00 equiv) in 1,4-dioxane (1.00 mL) and DMF (1.00 mL) was stirred for overnight at 50° C. under argon atmosphere. Desired product could be detected by LCMS. The resulting mixture was concentrated under reduced pressure. The crude product (mg) was purified by Prep-HPLC with the following conditions (Column: Xselect CSH C18 OBD Column 30*150 mm 5 μm, n; Mobile Phase A: Water (0. 1% FA), Mobile Phase B: ACN; Flow rate: 60 mL/min; Gradient: 17% B to 33% B in 9 min, 33% B; Wave Length: 254/220 nm; RT1 (min): 7.48) to afford 3-[(3-fluoro-2-methoxyphenyl)amino]-2-[6-[(1-methylcyclopropyl)amino]-1,5-naphthyridin-4-yl]-1H,5H,6H,7H-pyrrolo[3,2-c]pyridin-4-one; formic acid (30.8 mg, 24.48%) as a orange solid.

LC-MS: [M+H]⁺ found: 473.10.

¹H NMR (300 MHz, DMSO-d6) δ 14.07 (s, 1H), 8.27 (d, J=5.1 Hz, 1H), 8.13 (d, J=15.3 Hz, 2H), 7.99 (d, J=15.0 Hz, 1H), 7.67 (s, 1H), 7.41 (d, J=5.1 Hz, 1H), 7.23 (s, 1H), 7.02 (s, 1H), 6.72-6.69 (m, 1H), 6.62-6.56 (m, 1H), 6.05 (d, J=8.4 Hz, 1H), 3.98 (s, 3H), 3.47-3.44 (m, 2H), 3.00-2.92 (m, 2H), 1.62 (s, 3H), 0.89 (s, 4H).

### Example 215. 2-(6-cyclopropoxy-1,5-naphthyridin-4-yl)-3-[(3-fluoro-2-methoxyphenyl)amino]-1H,5H,6H,7H-pyrrolo[3,2-c]pyridin-4-one; trifluoroacetic acid (compound 696)

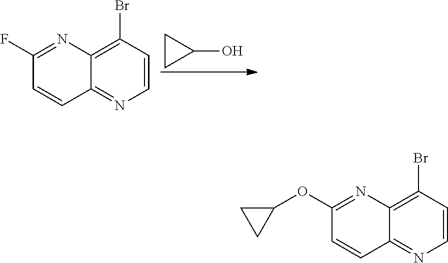

To a stirred solution of 8-bromo-2-fluoro-1,5-naphthyridine (300 mg, 1.32 mmol, 1 equiv) and cyclopropanol (383.73 mg, 6.60 mmol, 5 equiv) in THF (13 mL) was added t-BuOK (444.83 mg, 3.96 mmol, 3 equiv) in portions at 0° C. under argon atmosphere. The resulting mixture was stirred for 1 h at room temperature under argon atmosphere. The reaction was monitored by LCMS. The resulting mixture was concentrated under reduced pressure. The residue was purified by silica gel column chromatography, eluted with CH2Cl2/MeOH (100:1) to afford 8-bromo-2-cyclopropoxy-1,5-naphthyridine (360 mg, 99.89%) as an off-white solid.

LC-MS: (M+H)⁺ found: 266.8.

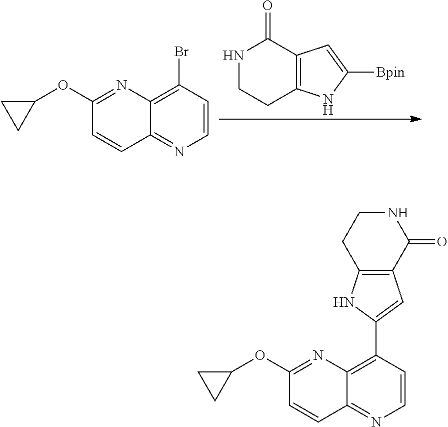

To a stirred mixture of 8-bromo-2-cyclopropoxy-1,5-naphthyridine (670 mg, 2.527 mmol, 1 equiv) and 2-(4,4,5,5-tetramethyl-1,3,2-dioxaborolan-2-yl)-1H,5H,6H,7H-pyrrolo[3,2-c]pyridin-4-one (794.93 mg, 3.032 mmol, 1.2 equiv) in DME (4 mL) H₂O (0.1 mL) was added Pd(dppf)Cl₂·CH₂Cl₂ (205.88 mg, 0.253 mmol, 0.1 equiv) and TEA (639.35 mg, 6.318 mmol, 2.5 equiv) at room temperature under argon atmosphere. The resulting mixture was stirred for overnight at 50° C. under argon atmosphere. The reaction was monitored by LCMS. The mixture was allowed to cool down to room temperature. The resulting mixture was extracted with EtOAc (3×50 mL). The combined organic layers were washed with water (3×10 mL), dried over anhydrous Na₂SO₄. After filtration, the filtrate was concentrated under reduced pressure. The residue was purified by silica gel column chromatography, eluted with CH₂Cl₂/MeOH (10:1) to afford 2-(6-cyclopropoxy-1,5-naphthyridin-4-yl)-1H,5H,6H,7H-pyrrolo[3,2-c]pyridin-4-one (700 mg, 77.86%) as a off-white solid.

LC-MS: (M+H)⁺ found: 321.1.

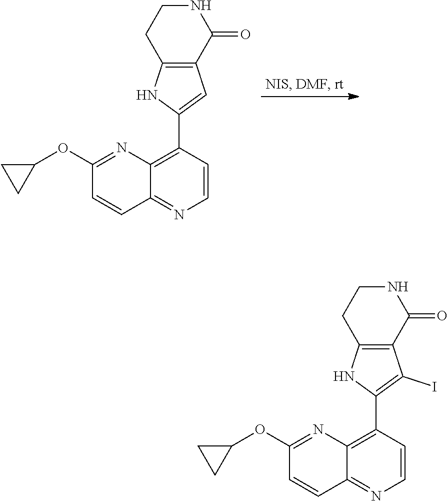 [SMILES: O=C1NCCc2[nH]c(-c3ccnc4ccc(OC5CC5)nc34)cc21 ; O=C1NCCc2[nH]c(-c3ccnc4ccc(OC5CC5)nc34)c(I)c21]

To a stirred mixture of 2-(6-cyclopropoxy-1,5-naphthyridin-4-yl)-1H,5H,6H,7H-pyrrolo[3,2-c]pyridin-4-one (400 mg, 1.12 mmol, 1 equiv) in DMF (11 mL, 142.14 mmol, 126.48 equiv) was added NIS (303.40 mg, 1.35 mmol, 1.2 equiv) in portions at room temperature under argon atmosphere. The resulting mixture was stirred for 2 h at room temperature under argon atmosphere. The reaction was monitored by LCMS. The reaction was quenched with sat. sodium sulfite (aq.) at room temperature. The precipitated solids were collected by filtration and washed with water (3×10 mL).

LC-MS: (M+H)⁺ found: 446.85.

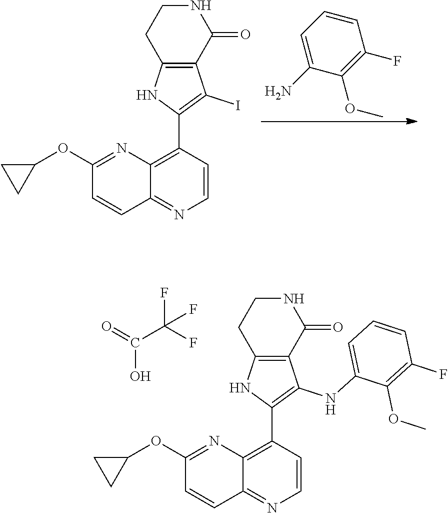

To a stirred mixture of 2-(6-cyclopropoxy-1,5-naphthyridin-4-yl)-3-iodo-1H,5H,6H,7H-pyrrolo[3,2-c]pyridin-4-one (200 mg, 0.45 mmol, 1.00 equiv) and 3-fluoro-2-methoxyaniline (75.91 mg, 0.54 mmol, 1.2 equiv) in dioxane (5.00 mL) was added EPhos Pd G4 (41.17 mg, 0.045 mmol, 0.10 equiv) and Cs₂CO₃ (438.08 mg, 1.34 mmol, 3.00 equiv) at room temperature under argon atmosphere. The resulting mixture was stirred for overnight at 50° C. under argon atmosphere. The reaction was monitored by LCMS. The mixture was allowed to cool down to room temperature. The resulting mixture was concentrated under reduced pressure. The crude product (300 mg) was purified by Prep-HPLC with the following conditions (Column: Xselect CSH C18 OBD Column 30*150 mm 5 μm, n; Mobile Phase A: Water (0.05% TFA), Mobile Phase B: ACN; Flow rate: 60 mL/min; Gradient: 23% B to 40% B in 8 min, 40% B; Wave Length: 254/220 nm; RT1 (min): 7.2) to afford 2-(6-cyclopropoxy-1,5-naphthyridin-4-yl)-3-[(3-fluoro-2-methoxyphenyl)amino]-1H,5H,6H,7H-pyrrolo[3,2-c]pyridin-4-one; trifluoroacetic acid (56.3 mg, 21.68%) as an orange solid.

LC-MS: (M+H)⁺ found: 460.05.

¹H NMR (400 MHz, DMSO-d6) δ 12.82 (s, 1H), 8.65 (d, J=5.5 Hz, 1H), 8.36 (d, J=9.2 Hz, 1H), 8.05 (s, 1H), 7.52-7.43 (m, 2H), 7.40 (s, 1H), 6.73 (td, J=8.3, 6.1 Hz, 1H), 6.65 (ddd, J=10.1, 8.4, 1.5 Hz, 1H), 6.07 (dt, J=8.0, 1.3 Hz, 1H), 4.70-4.58 (m, 1H), 3.98 (s, 3H), 3.48 (td, J=6.8, 2.3 Hz, 2H), 3.01 (t, J=6.8 Hz, 2H), 0.99 (td, J=5.2, 4.4, 2.5 Hz, 2H), 0.93 (dq, J=7.8, 3.5 Hz, 2H).

### Example 216. 2-(2-aminopyrimidin-4-yl)-3-[(2-ethoxy-3-fluorophenyl)amino]-1H,5H,6H,7H-pyrrolo[3,2-c]pyridin-4-one; trifluoroacetic acid (compound 699)

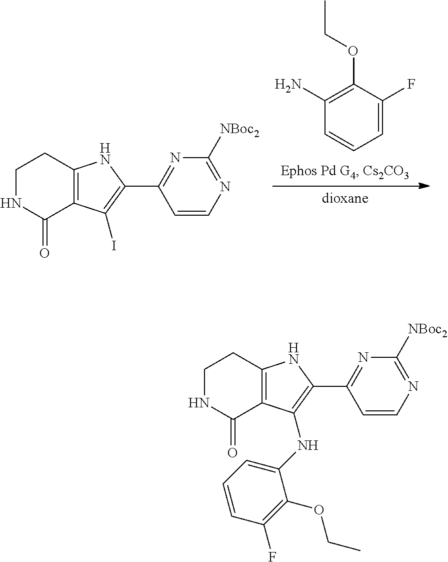

To a stirred mixture of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100.00 mg, 0.180 mmol, 1.00 equiv) and 2-ethoxy-3-fluoroaniline (33.53 mg, 0.216 mmol, 1.20 equiv) in dioxane (1.80 mL) were added Ephos Pd G4 (33.08 mg, 0.036 mmol, 0.20 equiv) and Cs2CO3 (117.33 mg, 0.360 mmol, 2.00 equiv) in portions at room temperature under nitrogen atmosphere. The resulting mixture was stirred for 2 h at 50 degrees C. under nitrogen atmosphere. Desired product could be detected by LCMS. The resulting mixture was filtered, the filter cake was washed with MeOH (3×5 mL). The filtrate was concentrated under reduced pressure. The residue was purified by silica gel column chromatography, eluted with CH2Cl2/MeOH (50:1) to afford tert-butyl N-(tert-butoxycarbonyl)-N-(4-[3-[(2-ethoxy-3-fluorophenyl)amino]-4-oxo-1H,5H,6H,7H-pyrrolo[3,2-c]pyridin-2-yl]pyrimidin-2-yl)carbamate (66 mg) as a light yellow solid.

LC-MS: M+H found: 583.15.

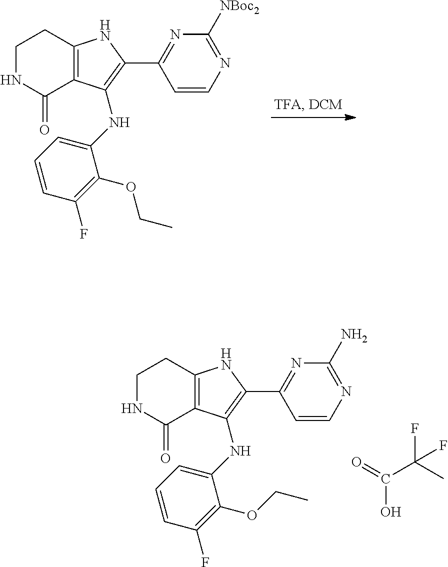

To a stirred solution of tert-butyl N-(tert-butoxycarbonyl)-N-(4-[3-[(2-ethoxy-3-fluorophenyl)amino]-4-oxo-1H,5H,6H,7H-pyrrolo[3,2-c]pyridin-2-yl]pyrimidin-2-yl)carbamate (66.00 mg, 0.113 mmol, 1.00 equiv) in DCM (1.13 mL) was added TFA (1.13 mL) dropwise at room temperature under nitrogen atmosphere. The resulting mixture was stirred for 2 h at room temperature under nitrogen atmosphere. Desired product could be detected by LCMS. The crude product was purified by Prep-HPLC with the following conditions (Column: Xselect CSH OBD Column 30*150 mm 5 um, n; Mobile Phase A: Water (0.05% TFA), Mobile Phase B: ACN; Flow rate: 60 mL/min; Gradient: 12% B to 30% B in 8 min, 30% B; Wave Length: 254/220 nm; RT1 (min): 8) to afford 2-(2-aminopyrimidin-4-yl)-3-[(2-ethoxy-3-fluorophenyl)amino]-1H,5H,6H,7H-pyrrolo[3,2-c]pyridin-4-one; trifluoroacetic acid (34.8 mg, 61.88%) as a yellow solid.

LC-MS: (M+H)⁺ found: 383.05.

¹H NMR (300 MHz, DMSO-d6) δ 12.01 (s, 1H), 8.04 (d, J=6.7 Hz, 1H), 7.62 (s, 2H), 7.31 (s, 1H), 7.00-6.05 (m, 4H), 4.13 (q, J=7.0 Hz, 2H), 3.41 (td, J=6.7, 2.5 Hz, 2H), 2.89 (t, J=6.6 Hz, 2H), 1.35 (t, J=7.0 Hz, 3H).

¹⁹F NMR (282 MHz, DMSO) δ−73.98, −131.81.

### Example 217. 2-(2-aminopyrimidin-4-yl)-3-[(3-chloro-2-ethoxyphenyl)amino]-1H,5H,6H,7H-pyrrolo[3,2-c]pyridin-4-one; trifluoroacetic acid (compound 700)

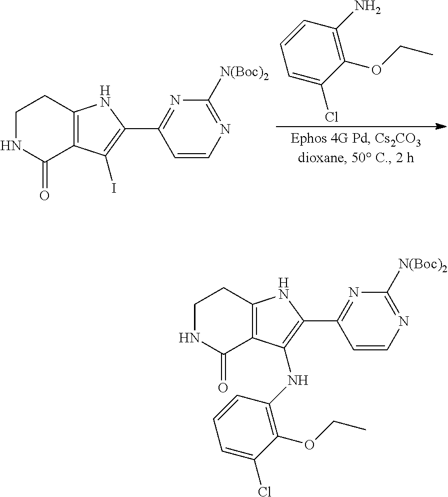

To a stirred mixture of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100.00 mg, 0.180 mmol, 1.00 equiv) and 3-chloro-2-ethoxyaniline (30.90 mg, 0.180 mmol, 1 equiv) in 1,4-dioxane (1.80 mL) was added EPhos Pd G4 (33.08 mg, 0.036 mmol, 0.2 equiv) and Cs₂CO₃ (234.67 mg, 0.720 mmol, 4 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford tert-butyl N-(tert-butoxycarbonyl)-N-(4-[3-[(3-chloro-2-ethoxyphenyl)amino]-4-oxo-1H,5H,6H,7H-pyrrolo[3,2-c]pyridin-2-yl]pyrimidin-2-yl)carbamate (70 mg, 64.89%) as a yellow solid.

LC-MS: (M+H)⁺ found: 599.15.

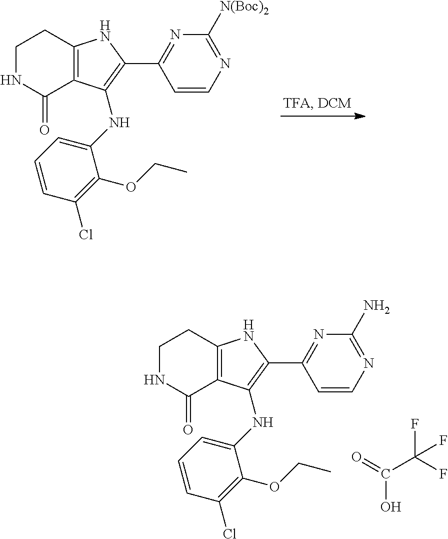

To a stirred mixture of tert-butyl N-(tert-butoxycarbonyl)-N-(4-[3-[(3-chloro-2-ethoxyphenyl)amino]-4-oxo-1H,5H,6H,7H-pyrrolo[3,2-c]pyridin-2-yl]pyrimidin-2-yl)carbamate (53.00 mg, 0.088 mmol, 1.00 equiv) in DCM (2 mL) was added TFA (1 mL) at room temperature under nitrogen atmosphere. The resulting mixture was stirred for 1 h and concentrated under reduced pressure. The crude product (40 mg) was purified by Prep-HPLC with the following conditions (Column: Xselect CSH C18 OBD Column 30*150 mm 5 μm; Mobile Phase A: Water (0.05% TFA), Mobile Phase B: ACN; Flow rate: 60 mL/min; Gradient: 15% B to 34% B in 8 min; Wave Length: 254/220 nm; RT1 (min): 7.5) to afford 2-(2-aminopyrimidin-4-yl)-3-[(3-chloro-2-ethoxyphenyl)amino]-1H,5H,6H,7H-pyrrolo[3,2-c]pyridin-4-one; trifluoroacetic acid (25.2 mg, 55.15%) as a yellow solid.

LC-MS: (M+H)⁺ found: 399.30.

¹H NMR (400 MHz, DMSO-d6): δ 11.94 (s, 1H), 8.46 (s, 1H), 8.03 (d, J=6.4 Hz, 1H), 7.30 (s, 2H), 7.26 (s, 1H), 6.91-6.84 (m, 2H), 6.62-6.53 (m, 2H), 6.11-6.06 (m, 2H), 3.42-3.39 (m, 2H), 2.87 (t, J=6.6 Hz, 2H), 1.38 (t, J=3.5 Hz, 3H).

### Example 218. 3-[(3-chloro-2-methoxyphenyl)amino]-2-[2-[(2,2,2-trifluoroethyl)amino]pyrimidin-4-yl]-1H,5H,6H,7H-pyrrolo[3,2-c]pyridin-4-one (compound 701)

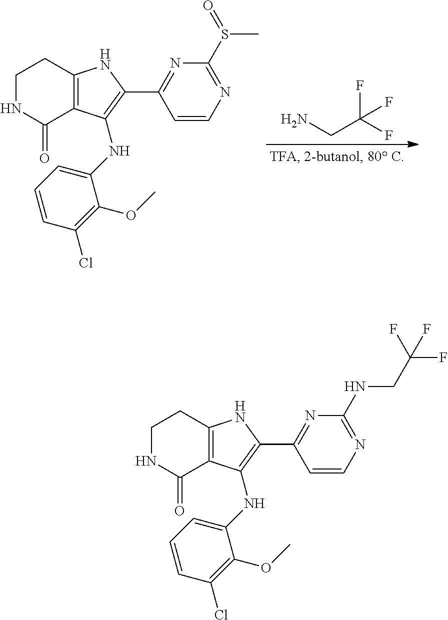 [SMILES: COc1c(Cl)cccc1Nc1c(-c2ccnc(S(C)=O)n2)[nH]c2c1C(=O)NCC2 ; NCC(F)(F)F ; COc1c(Cl)cccc1Nc1c(-c2ccnc(NCC(F)(F)F)n2)[nH]c2c1C(=O)NCC2]

Into a 8-mL sealed tube, was placed 3-[(3-chloro-2-methoxyphenyl)amino]-2-(2-methanesulfinylpyrimidin-4-yl)-1H,5H,6H,7H-pyrrolo[3,2-c]pyridin-4-one (130.00 mg, 0.301 mmol, 1.00 equiv), 2-butanol (3.00 mL), 2,2,2-trifluoroethylamine (178.89 mg, 1.806 mmol, 6.00 equiv), TFA (68.64 mg, 0.602 mmol, 2.00 equiv). The resulting solution was stirred overnight at 80 degrees C. The reaction was monitored by LCMS. Desired product could be detected by LCMS. The resulting mixture was concentrated under reduced pressure. The crude product (130 mg) was purified by Prep-HPLC with the following conditions (Column: Xselect CSH F-Phenyl OBD column, 19*250 mm, 5|Ìm; Mobile Phase A: Water (0.05% FA), Mobile Phase B: ACN; Flow rate: 25 mL/min; Gradient: 25% B to 35% B in 10 min, 35% B; Wave Length: 254 nm; RT1 (min): 8.83) to afford 3-[(3-chloro-2-methoxyphenyl)amino]-2-[2-[(2,2,2-trifluoroethyl)amino]pyrimidin-4-yl]-1H,5H,6H,7H-pyrrolo[3,2-c]pyridin-4-one (55.6 mg, 38.62%) as a yellow solid.

LC-MS: (M+H)⁺ found: 467.05.

¹H NMR (300 MHz, DMSO-d6) δ 11.89 (s, 1H), 8.14 (d, J=5.8 Hz, 1H), 7.94 (s, 1H), 7.28 (s, 1H), 6.91-6.78 (m, 2H), 6.62 (d, J=5.8 Hz, 1H), 6.37 (dd, J=6.5, 3.1 Hz, 1H), 4.36-4.24 (m, 2H), 3.89 (s, 3H), 3.43 (dt, J=6.3, 3.8 Hz, 2H), 2.93 (t, J=6.8 Hz, 2H).

### Example 219. 3-[(3-chloro-2-methoxyphenyl)amino]-2-[2-[(2,2-difluoroethyl)amino]pyrimidin-4-yl]-1H,5H,6H,7H-pyrrolo[3,2-c]pyridin-4-one (compound 702)

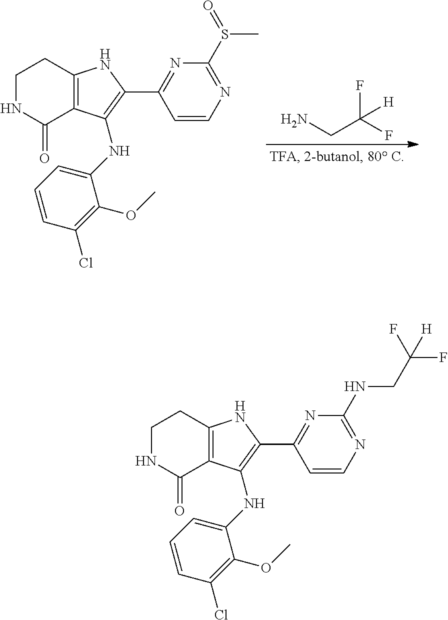 [SMILES: COc1c(Cl)cccc1Nc1c(-c2ccnc(S(C)=O)n2)[nH]c2c1C(=O)NCC2 ; NCC(F)F ; COc1c(Cl)cccc1Nc1c(-c2ccnc(NCC(F)F)n2)[nH]c2c1C(=O)NCC2]

Into a 8-mL sealed tube, was placed 3-[(3-chloro-2-methoxyphenyl)amino]-2-(2-methanesulfinylpyrimidin-4-yl)-1H,5H,6H,7H-pyrrolo[3,2-c]pyridin-4-one (150.00 mg, 0.347 mmol, 1.00 equiv), 2-butanol (3.50 mL), 2,2-difluoroethanamine (281.54 mg, 0.000 mmol, 10.00 equiv), TFA (118.80 mg, 1.041 mmol, 3.00 equiv). The resulting solution was stirred overnight at 80 degrees C. The reaction was monitored by LCMS. Desired product could be detected by LCMS. The resulting mixture was concentrated under reduced pressure. The crude product (150 mg) was purified by Prep-HPLC with the following conditions (Column: Xselect CSH C18 OBD Column 30*150 mm 5 μm; Mobile Phase A: Water (0.1% FA), Mobile Phase B: ACN; Flow rate: 60 mL/min; Gradient: 23% B to 33% B in 8 min; Wave Length: 254/220 nm; RT1 (min): 7.9) to afford 3-[(3-chloro-2-methoxyphenyl)amino]-2-[2-[(2,2-difluoroethyl)amino]pyrimidin-4-yl]-1H,5H,6H,7H-pyrrolo[3,2-c]pyridin-4-one (23.9 mg, 14.89%) as a yellow solid.

LC-MS: (M+H)⁺ found 449.05.

¹H NMR (300 MHz, DMSO-d6) δ 11.66 (s, 1H), 8.17-8.07 (m, 1H), 7.76 (s, 1H), 7.18 (d, J=2.7 Hz, 2H), 6.88-6.73 (m, 2H), 6.53 (d, J=5.3 Hz, 1H), 6.37-6.22 (m, 1H), 6.07 (t, J=4.3 Hz, 1H), 3.89 (s, 3H), 3.76 (dq, J=15.2, 5.0 Hz, 2H), 3.42 (td, J=6.8, 2.5 Hz, 2H), 2.89 (t, J=6.7 Hz, 2H).

### Example 220. 2-(2-aminopyrimidin-4-yl)-3-[(3-ethynyl-2-methoxyphenyl)amino]-1H,5H,6H,7H-pyrrolo[3,2-c]pyridin-4-one (compound 703)

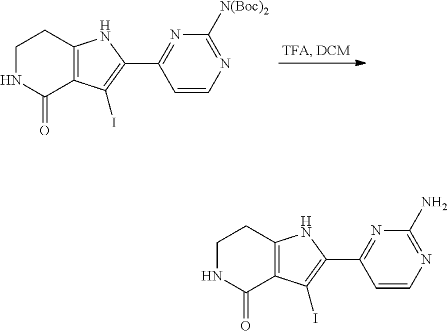

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (200.00 mg) in DCM (3 mL) was added TFA (1.5 mL) dropwise at room temperature under air. The resulting mixture was stirred for 1 h at room temperature. Desired product could be detected by LCMS. The resulting mixture was concentrated under vacuum. The residue was basified with saturated NaHCO₃ (aq.). The resulting mixture was extracted with CH₂Cl2:MeOH (3×20 mL) to give 2-(2-aminopyrimidin-4-yl)-3-iodo-1H,5H,6H,7H-pyrrolo[3,2-c]pyridin-4-one (69 mg) as brown solid.

LC-MS: (M+H)⁺ found: 356.05.

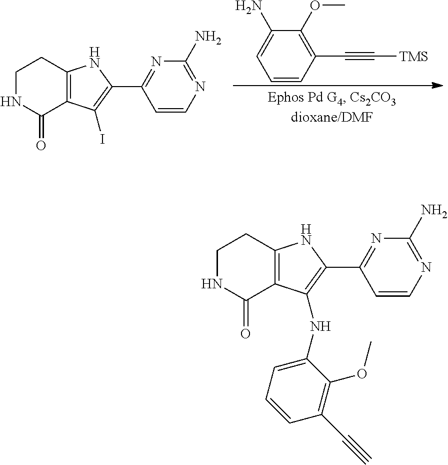 [SMILES: Nc1nccc(-c2[nH]c3c(c2I)C(=O)NCC3)n1 ; COc1c(N)cccc1C#C[Si](C)(C)C ; C#Cc1cccc(Nc2c(-c3ccnc(N)n3)[nH]c3c2C(=O)NCC3)c1OC]

To a solution of 2-(2-aminopyrimidin-4-yl)-3-iodo-1H,5H,6H,7H-pyrrolo[3,2-c] pyridin-4-one (60.00 mg, 0.113 mmol, 1.00 equiv) in dioxane (1.00 mL) and DMF (1.00 mL) was added 2-methoxy-3-[2-(trimethylsilyl)ethynyl]aniline (37.06 mg, 0.169 mmol, 1.5 equiv), Ephos Pd G4 (31.04 mg, 0.034 mmol, 0.30 equiv), Cs₂CO₃ (73.40 mg, 0.225 mmol, 2 equiv) in sequence. Desired product could be detected by LC-MS after 3 hours. The reaction mixture was concentrated under vacuum and purified by Prep-TLC (CH₂Cl₂/MeOH 10:1). The crude product (40 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 15% B to 45% B in 7 min, 45% B; Wave Length: 254 nm; RT1 (min): 6.5) to afford 2-(2-aminopyrimidin-4-yl)-3-[(3-ethynyl-2-methoxyphenyl)amino]-1H,5H,6H,7H-pyrrolo[3,2-c]pyridin-4-one (3.2 mg, 7.21%) as a yellow solid.

LC-MS: (M+H)⁺ found: 375.10.

¹H NMR (400 MHz, CD3OD) δ 7.95 (d, 1H), 6.81 (m, 2H), 6.51 (m, 2H), 4.10 (s, 3H), 3.55 (t, 2H), 2.97 (t, 2H).

### Example 221. 2-(2-aminopyrimidin-4-yl)-3-[[2-methoxy-3-(prop-1-yn-1-yl)phenyl]amino]-1H,5H,6H,7H-pyrrolo[3,2-c]pyridin-4-one (compound 704)

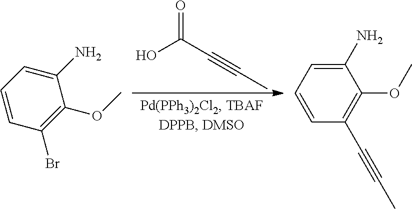

Into a 8-mL vial purged and maintained with an inert atmosphere of argon, was placed 3-bromo-2-methoxyaniline (250.00 mg, 1.237 mmol, 1.00 equiv), DMSO (2.50 mL), Pd(PPh₃)₂Cl₂ (86.85 mg, 0.124 mmol, 0.1 equiv), DPPB (121.37 mg, 0.285 mmol, 0.23 equiv), 2-butynoic acid (104.03 mg, 1.237 mmol, 1 equiv), TBAF (647.02 mg, 2.475 mmol, 2 equiv). The resulting solution was stirred for 2 hours at 110 degrees C. Desired product could be detected by LCMS. The resulting mixture was diluted with EtOAc (50 mL). The resulting mixture was washed with 50 mL of sat. NH4Cl (aq.). The resulting mixture was washed with 50 mL of water. The residue was purified by Prep-TLC (PE/EtOAc 4:1) to afford 2-methoxy-3-(prop-1-yn-1-yl) aniline (170 mg, 68.18%) as a light yellow oil.

LC-MS: (M+H)⁺ found: 162.

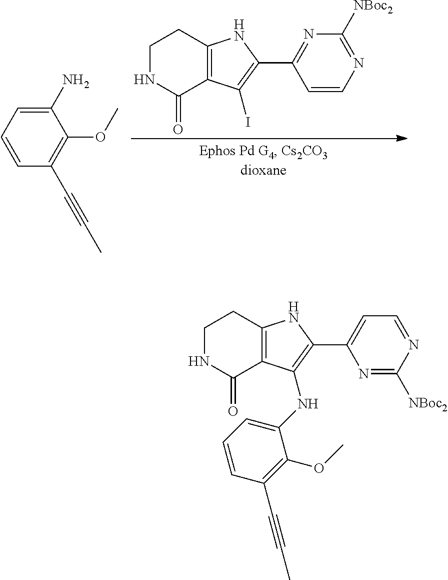

Into a 8-mL vial purged and maintained with an inert atmosphere of argon, was placed tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100.00 mg, 0.180 mmol, 1.00 equiv), dioxane (1.50 mL), 2-methoxy-3-(prop-1-yn-1-yl) aniline (43.54 mg, 0.270 mmol, 1.5 equiv), Ephos Pd G4 (33.08 mg, 0.036 mmol, 0.20 equiv), Cs₂CO₃ (117.33 mg, 0.360 mmol, 2.00 equiv). The resulting solution was stirred for 4 hours at 50 degrees C. Desired product could be detected by LCMS. The resulting mixture was filtered, the filter cake was washed with MeOH (2×10 mL). The filtrate was concentrated under reduced pressure. The residue was purified by Prep-TLC (CH₂Cl₂/MeOH 30:1) to afford tert-butyl N-(tert-butoxycarbonyl)-N-[4-(3-[[2-methoxy-3-(prop-1-yn-1-yl)phenyl]amino]-4-oxo-1H,5H,6H,7H-pyrrolo[3,2-c]pyridin-2-yl)pyrimidin-2-yl]carbamate (70 mg, 62.74%) as a red oil.

LC-MS (M+H)⁺ found: 589.

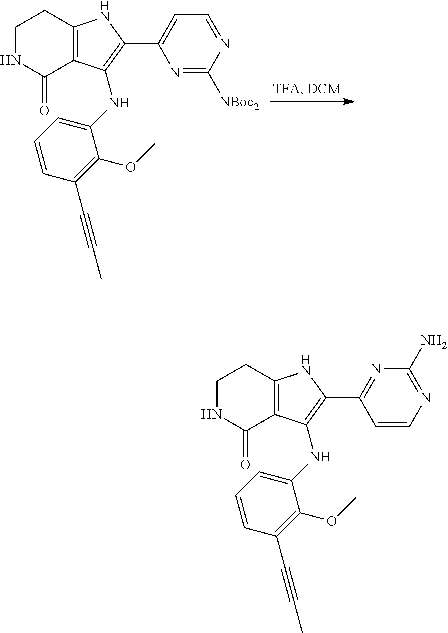

Into a 50-mL round-bottom flask, was placed tert-butyl N-(tert-butoxycarbonyl)-N-[4-(3-[[2-methoxy-3-(prop-1-yn-1-yl)phenyl]amino]-4-oxo-1H,5H,6H,7H-pyrrolo[3,2-c]pyridin-2-yl)pyrimidin-2-yl]carbamate (70.00 mg, 0.119 mmol, 1.00 equiv), DCM (2.50 mL), TFA (2.50 mL). The resulting solution was stirred for 2 hours at room temperature. Desired product could be detected by LCMS. The resulting mixture was concentrated under reduced pressure. The crude product (100 mg) was purified by Prep-HPLC with the following conditions (Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 18% B to 48% B in 7 min; Wave Length: 254 nm; RT1 (min): 6.5) to afford 2-(2-aminopyrimidin-4-yl)-3-[[2-methoxy-3-(prop-1-yn-1-yl)phenyl]amino]-1H,5H,6H,7H-pyrrolo[3,2-c]pyridin-4-one (26.6 mg, 55.86%) as a white solid.

LC-MS (M+H)⁺ found: 389.

¹H NMR (300 MHz, DMSO-d6) 11.61 (s, 1H), 8.05-8.03 (m, 1H), 7.94 (s, 1H), 7.15 (s, 1H), 7.29 (s, 1H), 6.78-6.69 (d, 2H), 6.44-6.36 (d, 2H), 6.18 (s, 2H), 3.95 (s, 3H), 3.39-3.37 (d, 2H), 2.86-2.82 (t, 2H), 2.09 (s, 3H).

### Example 222. 2-(2-aminopyrimidin-4-yl)-3-[(3-methoxy-2-methylphenyl)amino]-1H,5H,6H,7H-pyrrolo[3,2-c]pyridin-4-one (compound 705)

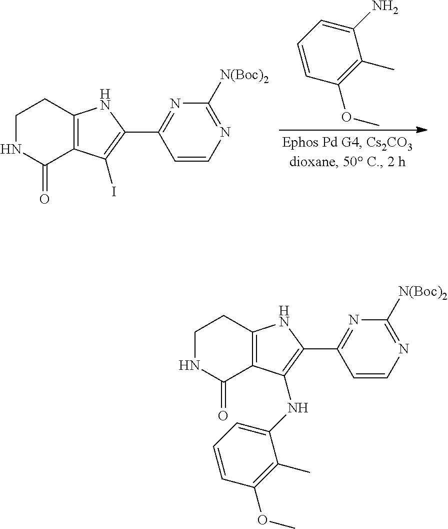

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100 mg, 0.18 mmol, 1.00 equiv) in dioxane (2.00 mL) were added Cs₂CO₃ (117 mg, 0.36 mmol, 2 equiv), Ephos Pd G4 (33 mg, 0.036 mmol, 0.2 equiv) and 3-methoxy-2-methylaniline (30 mg, 0.22 mmol, 1.20 equiv). The mixture was stirred for 2 h at 50 degrees C. under nitrogen atmosphere. The reaction was monitored by LCMS. The resulting mixture was concentrated under reduced pressure. The residue was purified by Prep-TLC (CH₂Cl2/MeOH 15:1) to afford tert-butyl N-(tert-butoxycarbonyl)-N-(4-[3-[(3-methoxy-2-methylphenyl)amino]-4-oxo-1H,5H,6H,7H-pyrrolo[3,2-c]pyridin-2-yl]pyrimidin-2-yl)carbamate (59 mg, 58.03%) as a yellow oil.

LC-MS: (M+H)⁺ found: 565.20.

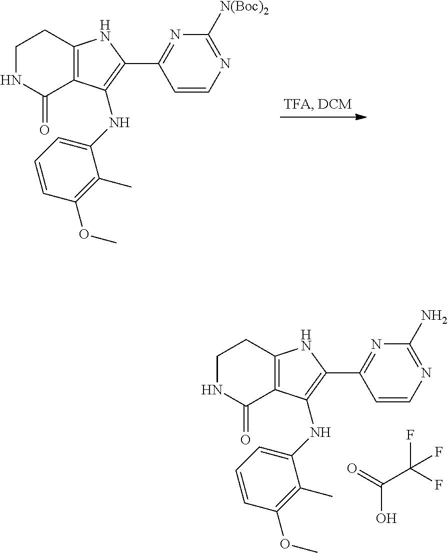

To a stirred solution of tert-butyl N-(tert-butoxycarbonyl)-N-(4-[3-[(3-methoxy-2-methylphenyl)amino]-4-oxo-1H,5H,6H,7H-pyrrolo[3,2-c]pyridin-2-yl]pyrimidin-2-yl)carbamate) in DCM (2.00 mL) was added TFA (1.00 mL) in portions. The mixture was stirred for 1 h at room temperature under nitrogen atmosphere. The reaction was monitored by LCMS. The resulting mixture was concentrated under vacuum. The crude product (mg) was purified by Prep-HPLC with the following conditions (Column: Xselect CSH C18 OBD Column 30*150 mm 5 μm; Mobile Phase A: Water (0.05% TFA), Mobile Phase B: MeOH; Flow rate: 60 mL/min; Gradient: 23% B to 49/a B in 8 min; Wave Length: 254/220 nm; RT1 (min): 8) to afford 2-(2-aminopyrimidin-4-yl)-3-[(3-methoxy-2-methylphenyl)amino]-1H,5H,6H,7H-pyrrolo[3,2-c]pyridin-4-one; trifluoroacetic acid (31.5 mg, 60.68%) as a yellow solid.

LC-MS: (M+H)⁺ found: 365.10.

¹H NMR (300 MHz, Methanol-d4): δ 7.68 (d, J=7.2 Hz, 1H), 6.94 (t, J=8.4 Hz, 1H), 6.60 (d, J=8.4 Hz, 1H), 6.47 (d, J=8.1 Hz, 2H), 3.82 (s, 3H), 3.60-3.54 (m, 2H), 3.01-2.97 (m, 2H), 2.23 (s, 3H).

### Example 223. 2-(2-aminopyrimidin-4-yl)-3-[(3-chloro-2-fluorophenyl)amino]-1H,5H,6H,7H-pyrrolo[3,2-c]pyridin-4-one (compound 706)

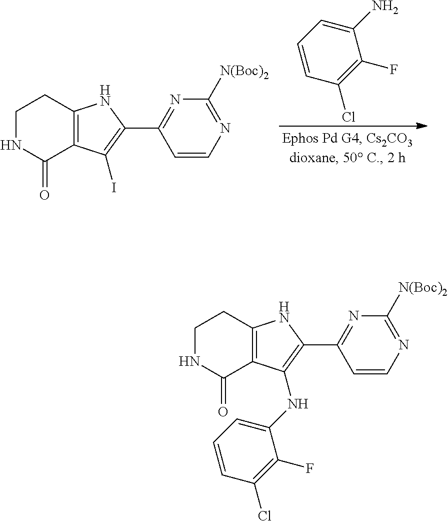

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100 mg, 0.18 mmol, 1.00 equiv) in Dioxane (2.00 mL) was added Cs₂CO₃ (118 mg, 0.36 mmol, 2.00 equiv), Ephos Pd G4 (33 mg, 0.04 mmol, 0.20 equiv) and 3-chloro-2-fluoroaniline (26 mg, 0.18 mmol, 1.00 equiv). The mixture was stirred for 2 h at 50 degrees C. under nitrogen atmosphere. The reaction was monitored by LCMS. The resulting mixture was concentrated under reduced pressure. The residue was purified by Prep-TLC (CH₂Cl₂/MeOH 15:1) to afford tert-butyl N-(tert-butoxycarbonyl)-N-(4-[3-[(3-chloro-2-fluorophenyl)amino]-4-oxo-1H,5H,6H,7H-pyrrolo[3,2-c]pyridin-2-yl]pyrimidin-2-yl)carbamate (65 mg, 63.00%) as a yellow oil.

LC-MS: (M+H)⁺ found: 573.15.

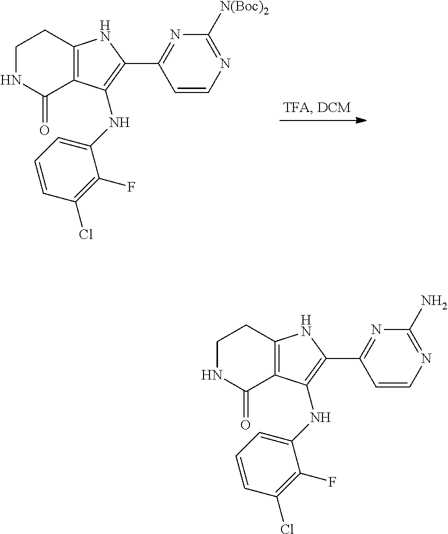

To a stirred solution of tert-butyl N-(tert-butoxycarbonyl)-N-(4-[3-[(3-chloro-2-fluorophenyl)amino]-4-oxo-1H,5H,6H,7H-pyrrolo[3,2-c]pyridin-2-yl]pyrimidin-2-yl)carbamate (65 mg, 0.11 mmol, 1.00 equiv) in DCM (2.00 mL) was added TFA (1.00 mL) in portions. The mixture was stirred for 1 h at room temperature under nitrogen atmosphere. The reaction was monitored by LCMS. The resulting mixture was concentrated under reduced pressure. The crude product (40 mg) was purified by Prep-HPLC with the following conditions (Column: Xselect CSH F-Phenyl OBD column, 19*250 mm, 5 μm; Mobile Phase A: Water (10 mmol/L NH4HCO3), Mobile Phase B: MeOH-Preparative; Flow rate: 25 m/min; Gradient: 56% B to 68% B in 7 min, 68% B; Wave Length: 254 nm; RT1 (min): 6.42) to afford 2-(2-aminopyrimidin-4-yl)-3-[(3-chloro-2-fluorophenyl)amino]-1H,5H,6H,7H-pyrrolo[3,2-c]pyridin-4-one (7.9 mg, 17.75%) as a white solid.

LC-MS: (M+H)⁺ found: 373.00.

¹H NMR (400 MHz, DMSO-d6): δ 11.71 (s, 1H), 8.25 (s, 1H), 8.10 (d, J=5.4 Hz, 1H), 7.11 (s, 1H), 6.89-6.82 (m, 2H), 6.63 (d, J=5.4 Hz, 1H), 6.57-6.52 (m, 1H), 6.29 (s, 2H), 3.41-3.34 (m, 2H), 2.84 (t, J=6.7 Hz, 2H).

### Example 224. 2-(2-amino-3-fluoropyridin-4-yl)-3-[(3-chloro-2-methoxyphenyl)amino]-1H,5H,6H,7H-pyrrolo[3,2-c]pyridin-4-one; trifluoroacetic acid (compound 686)

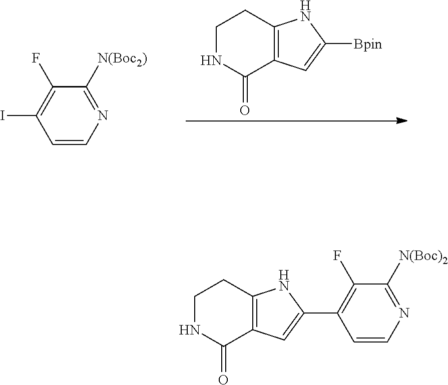

To a stirred solution of tert-butyl N-(tert-butoxycarbonyl)-N-(3-fluoro-4-iodopyridin-2-yl)carbamate (1.00 g, 2.28 mmol, 1.00 equiv) and 2-(4,4,5,5-tetramethyl-1,3,2-dioxaborolan-2-yl)-1H,5H,6H,7H-pyrrolo[3,2-c]pyridin-4-one (1.20 g, 4.56 mmol, 2.00 equiv) in dioxane (20.00 mL) were added Na₂CO₃ (725 mg, 6.84 mmol, 3.00 equiv) and Pd(PPh₃)₄ (263 mg, 0.22 mmol, 0.10 equiv) in portions at room temperature under nitrogen atmosphere. To the above mixture was added H₂O (4.00 mL) dropwise at room temperature. The resulting mixture was stirred for additional overnight at 50 degrees C. The reaction was monitored by LCMS. LCMS showed the reaction was completed. The resulting mixture was concentrated under reduced pressure. The residue was purified by silica gel column chromatography, eluted with CH₂Cl2/MeOH (10:1) to afford tert-butyl N-(tert-butoxycarbonyl)-N-(3-fluoro-4-[4-oxo-1H,5H,6H,7H-pyrrolo[3,2-c]pyridin-2-yl]pyridin-2-yl)carbamate (890 mg, 87.36%) as a grey solid.

LC-MS: (M+H)⁺ found: 477.20.

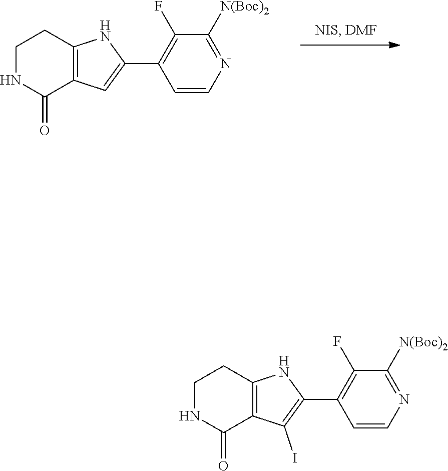

To a stirred solution of tert-butyl N-(tert-butoxycarbonyl)-N-(3-fluoro-4-[4-oxo-1H,5H,6H,7H-pyrrolo[3,2-c]pyridin-2-yl]pyridin-2-yl)carbamate (690 mg, 1.54 mmol, 1.00 equiv) in DMF (15.00 mL) was added N-iodosuccinimide (382 mg, 1.70 mmol, 1.10 equiv) dropwise at room temperature under nitrogen atmosphere. The reaction was monitored by LCMS. LCMS showed the reaction was completed. The reaction was quenched by the addition of Na2SO3 (IOmL) at room temperature. The mixture was neutralized to pH 7 with saturated Na2CO3 (aq.). The resulting mixture was extracted with CH₂Cl₂:MeOH (3×100 mL). The combined organic layers were washed with brine (2×300 mL), dried over anhydrous Na₂SO₄. After filtration, the filtrate was concentrated under reduced pressure. The residue was purified by silica gel column chromatography, eluted with CH2Cl2/MeOH (10:1) to afford tert-butyl N-(tert-butoxycarbonyl)-N-(3-fluoro-4-[3-iodo-4-oxo-1H,5H,6H,7H-pyrrolo[3,2-c]pyridin-2-yl]pyridin-2-yl)carbamate (317 mg, 35.84%) as an off-white solid.

LC-MS: (M+H)⁺ found: 573.1.

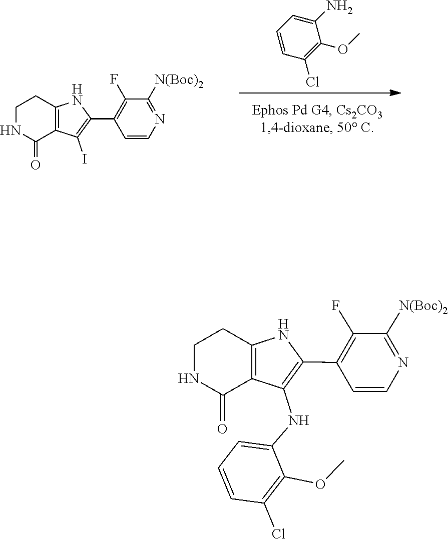

To a stirred solution of tert-butyl N-(tert-butoxycarbonyl)-N-(3-fluoro-4-[3-iodo-4-oxo-1H,5H,6H,7H-pyrrolo[3,2-c]pyridin-2-yl]pyridin-2-yl)carbamate (100 mg, 0.17 mmol, 1.00 equiv) in dioxane (1.80 mL) was added Cs₂CO₃ (113 mg, 0.34 mmol, 2.00 equiv) and EPhos Pd G4 (32 mg, 0.03 mmol, 0.20 equiv) in portions at room temperature under nitrogen atmosphere. To the above mixture was added 3-chloro-2-methoxyaniline (27 mg, 0.17 mmol, 1.00 equiv) in portions at room temperature. The resulting mixture was stirred for additional 3 h at 50 degrees C. The reaction was monitored by LCMS. LCMS showed the reaction was completed. The resulting mixture was concentrated under reduced pressure. The residue was purified by Prep-TLC (CH₂Cl2/MeOH 10:1) to afford tert-butyl N-(tert-butoxycarbonyl)-N-(4-[3-[(3-chloro-2-methoxyphenyl)amino]-4-oxo-1H,5H,6H,7H-pyrrolo[3,2-c]pyridin-2-yl]-3-fluoropyridin-2-yl)carbamate (50 mg, 47.53%) as a yellow solid.

LC-MS: (M+H)⁺ found: 602.05.

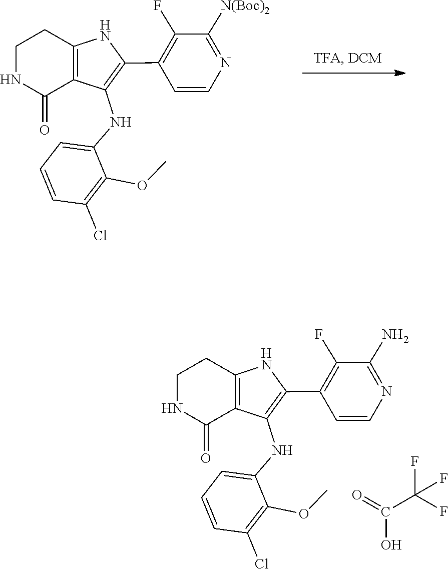

To a stirred solution of tert-butyl N-(tert-butoxycarbonyl)-N-(4-[3-[(3-chloro-2-methoxyphenyl)amino]-4-oxo-1H,5H,6H,7H-pyrrolo[3,2-c]pyridin-2-yl]-3-fluoropyridin-2-yl)carbamate (50 mg, 0.08 mmol, 1.00 equiv) in DCM (1.00 mL) was added TFA (1.00 mL) dropwise at room temperature under nitrogen atmosphere. The resulting mixture was stirred for additional 1 h at room temperature. The reaction was monitored by LCMS. LCMS showed the reaction was completed. The resulting mixture was concentrated under reduced pressure. The crude product (33 mg) was purified by Prep-HPLC with the following conditions (Column: Xselect CSH C18 OBD Column 30*150 mm 5 μm; Mobile Phase A: Water (0.05% TFA), Mobile Phase B: ACN; Flow rate: 60 mL/min; Gradient: 13% B to 30% B in 8 min; Wave Length: 254/220 nm; RT1 (min): 7.42) to afford 2-(2-amino-3-fluoropyridin-4-yl)-3-[(3-chloro-2-methoxyphenyl)amino]-1H,5H,6H,7H-pyrrolo[3,2-c]pyridin-4-one; trifluoroacetic acid (14.8 mg, 34.55%) as a yellow solid.

LC-MS: (M+H)⁺ found: 402.

¹H NMR (400 MHz, DMSO-d6) δ 11.67 (s, 1H), 7.71-7.62 (m, 4H), 7.26 (s, 1H), 6.79-6.72 (m, 3H), 6.23-6.20 (m, 1H), 3.87 (s, 3H), 3.43-3.39 (m, 2H), 2.87 (t, J=8.0 Hz, 2H).

### Example 225. 2-(2-aminopyrimidin-4-yl)-3-[(3-chloro-5-fluoro-2-methoxyphenyl)amino]-1H,5H,6H,7H-pyrrolo[3,2-c]pyridin-4-one; trifluoroacetic acid (compound 714)

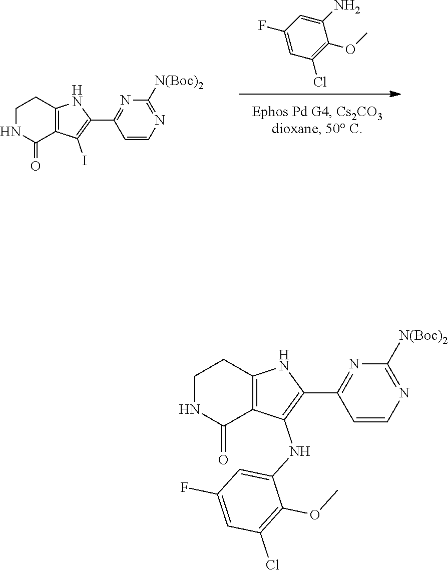

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100 mg, 0.18 mmol, 1.00 equiv) in dioxane (2.00 mL) was added EPhos Pd G4 (33 mg, 0.03 mmol, 0.20 equiv) and Cs₂CO₃ (117 mg, 0.36 mmol, 2.00 equiv) in portions at room temperature under nitrogen atmosphere. To the above mixture was added 3-chloro-5-fluoro-2-methoxyaniline (31 mg, 0.18 mmol, 1.00 equiv) dropwise at room temperature. The resulting mixture was stirred for additional 2 h at 50 degrees C. The reaction was monitored by LCMS. LCMS showed the reaction was completed. The resulting mixture was concentrated under vacuum. The residue was purified by Prep-TLC (CH₂Cl₂/MeOH 10:1) to afford tert-butyl N-(tert-butoxycarbonyl)-N-(4-[3-[(3-chloro-5-fluoro-2-methoxyphenyl)amino]-4-oxo-1H,5H,6H,7H-pyrrolo[3,2-c]pyridin-2-yl]pyrimidin-2-yl)carbamate (96 mg, 88.41%) as a brown solid.

LC-MS: (M+H)⁺ found: 603.05.

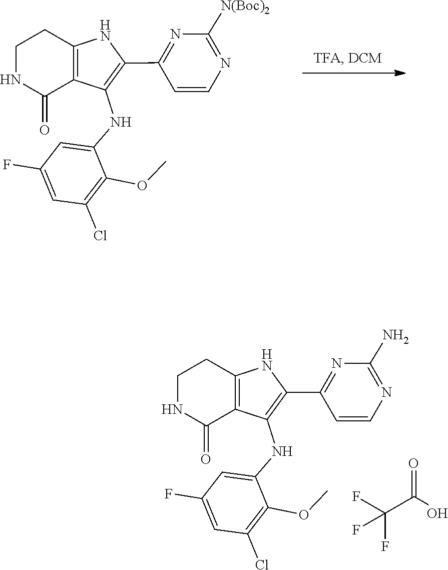

To a stirred solution of tert-butyl N-(tert-butoxycarbonyl)-N-(4-[3-[(3-chloro-5-fluoro-2-methoxyphenyl)amino]-4-oxo-1H,5H,6H,7H-pyrrolo[3,2-c]pyridin-2-yl]pyrimidin-2-yl)carbamate (60 mg, 0.09 mmol, 1.00 equiv) in DCM (1.00 mL) was added TFA (1.00 mL) dropwise at room temperature under nitrogen atmosphere. The resulting mixture was stirred for additional 2 h at 50 degrees C. The reaction was monitored by LCMS. LCMS showed the reaction was completed. The resulting mixture was concentrated under reduced pressure. The crude product (55 mg) was purified by Prep-HPLC with the following conditions (Column: Xselect CSH OBD Column 30*150 mm Sum, n; Mobile Phase A: Water (0.05% TFA), Mobile Phase B: ACN; Flow rate: 60 mL/min; Gradient: 15% B to 30% B in 8 min, 30% B: Wave Length: 254/220 nm; RT1 (min): 7.43) to afford 2-(2-aminopyrimidin-4-yl)-3-[(3-chloro-5-fluoro-2-methoxyphenyl)amino]-1H,5H,6H,7H-pyrrolo[3,2-c]pyridin-4-one; trifluoroacetic acid (37.7 mg, 73.31%) as a yellow solid.

LC-MS: (M+H)⁺ found: 403.

¹H NMR (400 MHz, DMSO-d6) δ 12.07 (s, 1H), 8.54 (s, 1H), 8.10 (d, J=4 Hz, 1H), 7.43-7.27 (m, 3H), 6.81-6.78 (m, 2H), 6.30-6.26 (m, JH), 3.83 (s, 3H), 3.43-3.39 (m, 2H), 2.89 (t, J=4 Hz, 2H).

### Example 226. 2-(2-aminopyrimidin-4-yl)-3-[[3-chloro-2-(methylsulfanyl)phenyl]amino]-1H,5H,6H,7H-pyrrolo[3,2-c]pyridin-4-one (compound 715)

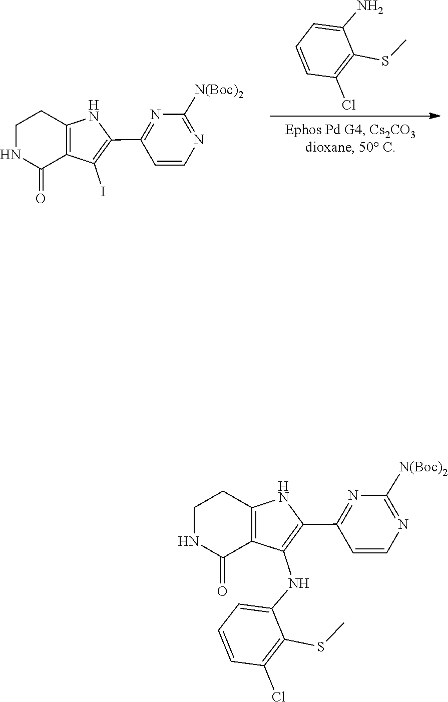

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100 mg, 0.18 mmol, 1.00 equiv) in dioxane (2.00 mL) was added EPhos Pd G4 (33 mg, 0.03 mmol, 0.20 equiv) and Cs₂CO₃ (117 mg, 0.36 mmol, 2.00 equiv) in portions at room temperature under nitrogen atmosphere. To the above mixture was added 3-chloro-2-(methylsulfanyl) aniline (31 mg, 0.18 mmol, 1.00 equiv) dropwise at room temperature. The resulting mixture was stirred for additional 2 h at 50 degrees C. The reaction was monitored by LCMS. LCMS showed the reaction was completed. The resulting mixture was concentrated under reduced pressure. The residue was purified by Prep-TLC (CH₂Cl2/MeOH 10:1) to afford tert-butyl N-(tert-butoxycarbonyl)-N-[4-(3-[[3-chloro-2-(methylsulfanyl)phenyl]amino]-4-oxo-1H,5H,6H,7H-pyrrolo[3,2-c]pyridin-2-yl)pyrimidin-2-yl]carbamate (42 mg, 38.80%) as a brown solid.

LC-MS: (M+H)⁺ found: 601.05.

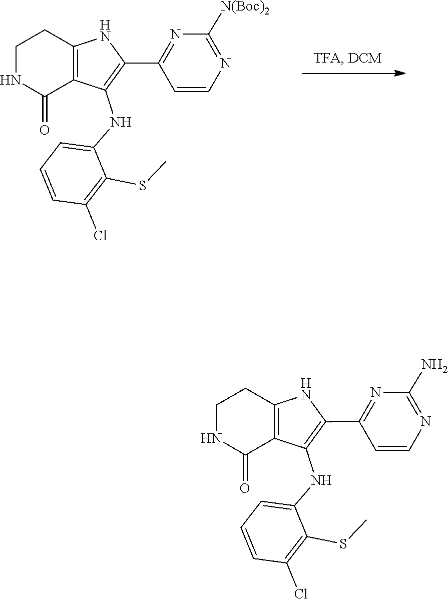

To a stirred solution of tert-butyl N-(tert-butoxycarbonyl)-N-[4-(3-[[3-chloro-2-(methylsulfanyl)phenyl]amino]-4-oxo-1H,5H,6H,7H-pyrrolo[3,2-c]pyridin-2-yl)pyiimidin-2-yl]carbamate (42 mg, 0.07 mmol, 1.00 equiv) in DCM (1.00 mL) was added TFA (1.00 mL) dropwise at 0 degrees C. under nitrogen atmosphere. The resulting mixture was stirred for additional 1 h at room temperature. The reaction was monitored by LCMS. LCMS showed the reaction was completed. The resulting mixture was concentrated under reduced pressure. The crude product (24 mg) was purified by Prep-HPLC with the following conditions (Column: XBridge Prep OBD C18 Column, 30|Á150 mm 5 um; Mobile Phase A: Water (10 mmol/L NH4HCO3), Mobile Phase B: ACN; Flow rate: 60 mL/min; Gradient: 22% B to 52% B in 7 min, 52% B; Wave Length: 254 nm; RT1 (min): 6.5) to afford 2-(2-aminopyrimidin-4-yl)-3-[[3-chloro-2-(methylsulfanyl)phenyl]amino]-1H,5H,6H,7H-pyrrolo[3,2-c]pyridin-4-one (1.4 mg, 5.00%) as a yellow solid.

LC-MS: (M+H)⁺ found: 401.

¹H NMR (400 MHz, DMSO-d6) δ 11.71 (s, 1H), 8.60 (s, 1H), 8.06 (d, J=4.0 Hz, 1H), 7.13 (s, 1H), 7.05 (t, J=8.0 Hz, 1H), 6.87-6.85 (m, 1H), 6.48 (d, J=4.0 Hz, 1H), 6.43-6.40 (m, 1H), 6.20 (s, 2H), 3.40-3.33 (m, 2H), 2.84 (t, J=8.0 Hz, 2H), 2.39 (s, 3H).

### Example 227. 2-(2-aminopyrimidin-4-yl)-3-[[3-fluoro-2-(methylsulfanyl)phenyl]amino]-1H,5H,6H,7H-pyrrolo[3,2-c]pyridin-4-one; trifluoroacetic acid (compound 716)

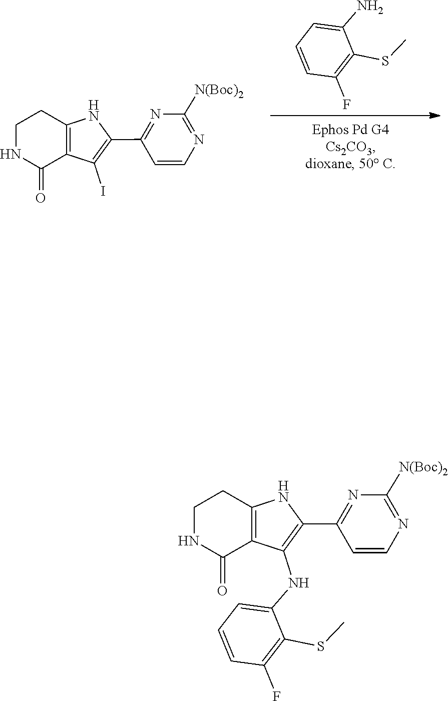

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100 mg, 0.18 mmol, 1.00 equiv) in dioxane (1.80 mL) was added EPhos Pd G4 (33 mg, 0.03 mmol, 0.20 equiv) and Cs₂CO₃ (117 mg, 0.36 mmol, 2.00 equiv) in portions at room temperature under nitrogen atmosphere. To the above mixture was added 3-fluoro-2-(methylsulfanyl) aniline (84 mg, 0.54 mmol, 3.00 equiv) dropwise at room temperature. The resulting mixture was stirred for additional 3 h at 50 degrees C. The reaction was monitored by LCMS. LCMS showed the reaction was completed. The resulting mixture was concentrated under reduced pressure. The residue was purified by silica gel column chromatography, eluted with CH₂Cl2/MeOH (10:1) to afford tert-butyl N-(tert-butoxycarbonyl)-N-[4-(3-[[3-fluoro-2-(methylsulfanyl)phenyl]amino]-4-oxo-1H,5H,6H,7H-pyrrolo[3,2-c]pyridin-2-yl)pyrimidin-2-yl]carbamate (90 mg, 85.49%) as a yellow solid.

LC-MS: (M+H)⁺ found: 585.

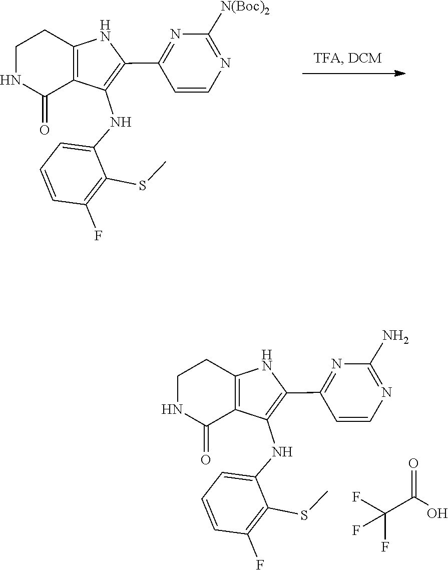

To a stirred solution of tert-butyl N-(tert-butoxycarbonyl)-N-[4-(3-[[3-fluoro-2-(methylsulfanyl)phenyl]amino]-4-oxo-1H,5H,6H,7H-pyrrolo[3,2-c]pyridin-2-yl)pyrimidin-2-yl]carbamate (90 mg, 0.15 mmol, 1.00 equiv) in DCM (1.00 mL) was added TFA (1.00 mL) dropwise at room temperature under nitrogen atmosphere. The resulting mixture was stirred for 3 h at room temperature under nitrogen atmosphere. The reaction was monitored by LCMS. LCMS showed the reaction was completed. The resulting mixture was concentrated under vacuum. The crude product (60 mg) was purified by Prep-HPLC with the following conditions (Column: Xselect CSH OBD Column 30*150 mm Sum, n; Mobile Phase A: Water (0.05% TFA), Mobile Phase B: ACN; Flow rate: 60 mL/min; Gradient: 12% B to 30% B in 8 min, 30% B; Wave Length: 254/220 nm; RT1 (min): 7.7) to afford 2-(2-aminopyrimidin-4-yl)-3-[[3-fluoro-2-(methylsulfanyl)phenyl]amino]-1H,5H,6H,7H-pyrrolo[3,2-c]pyridin-4-one; trifluoroacetic acid (45.5 mg, 59.30%) as a yellow solid.

LC-MS: (M+H)⁺ found: 385.

¹H NMR (400 MHz, DMSO-d6) δ 12.06 (s, 1H), 8.95 (s, 1H), 8.06 (d, J=4 Hz, 1H), 7.43 (s, 2H), 7.27 (s, 1H), 7.15-7.09 (m, 1H), 6.72-6.67 (m, 2H), 6.46 (d, J=8.0 Hz, 1H), 3.43-3.41 (m, 2H), 2.90 (t, J=8.0 Hz, 2H), 2.38 (s, 3H).

### Example 228. 2-(2-aminopyrimidin-4-yl)-3-[(3-chloro-2-fluorophenyl)amino]-1H,5H,6H,7H-pyrrolo[3,2-c]pyridin-4-one (compound 717)

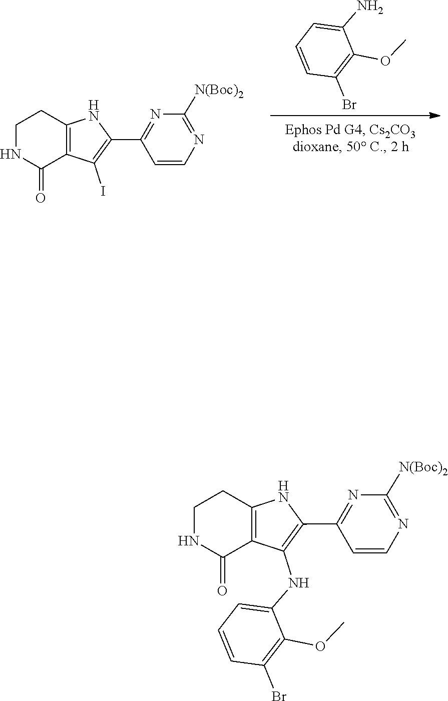

To a stirred solution of tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (100 mg, 0.18 mmol, 1.00 equiv) in Dioxane (2.00 mL) was added Cs₂CO₃ (118 mg, 0.36 mmol, 2.00 equiv), Ephos Pd G4 (33 mg, 0.04 mmol, 0.20 equiv) and 3-chloro-2-fluoroaniline (26 mg, 0.18 mmol, 1.00 equiv). The mixture was stirred for 2 h at 50 degrees C. under nitrogen atmosphere. The reaction was monitored by LCMS. The resulting mixture was concentrated under reduced pressure. The residue was purified by Prep-TLC (CH₂Cl₂/MeOH 15:1) to afford tert-butyl N-(tert-butoxycarbonyl)-N-(4-[3-[(3-chloro-2-fluorophenyl)amino]-4-oxo-1H,5H,6H,7H-pyrrolo[3,2-c]pyridin-2-yl]pyrimidin-2-yl)carbamate (65 mg, 63.00%) as a yellow oil.

LC-MS: (M+H)⁺ found: 573.15.

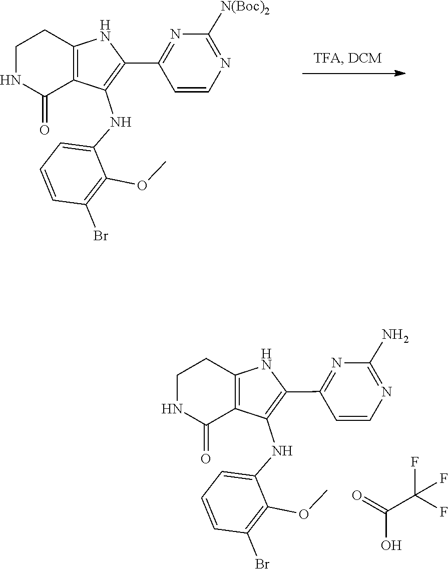

To a stirred solution of tert-butyl N-(tert-butoxycarbonyl)-N-(4-[3-[(3-chloro-2-fluorophenyl)amino]-4-oxo-1H,5H,6H,7H-pyrrolo[3,2-c]pyridin-2-yl]pyrimidin-2-yl)carbamate (65 mg, 0.11 mmol, 1.00 equiv) in DCM (2.00 mL) was added TFA (1.00 mL) in portions. The mixture was stirred for 1 h at room temperature under nitrogen atmosphere. The reaction was monitored by LCMS. The resulting mixture was concentrated under reduced pressure. The crude product (40 mg) was purified by Prep-HPLC with the following conditions (Column: Xselect CSH F-Phenyl OBD column, 19*250 mm, 5 μm; Mobile Phase A: Water (10 mmol/L NH4HCO3), Mobile Phase B: MeOH-Preparative; Flow rate: 25 mL/min; Gradient: 56% B to 68% B in 7 min, 68% B; Wave Length: 254 nm; RT1 (min): 6.42) to afford 2-(2-aminopyrimidin-4-yl)-3-[(3-chloro-2-fluorophenyl)amino]-1H,5H,6H,7H-pyrrolo[3,2-c]pyridin-4-one (7.9 mg, 17.75%) as a white solid.

LC-MS: (M+H)⁺ found: 373.00.

¹H NMR (400 MHz, DMSO-d6): δ 11.71 (s, 1H), 8.25 (s, 1H), 8.10 (d, J=5.4 Hz, 1H), 7.11 (s, 1H), 6.89-6.82 (m, 2H), 6.63 (d, J=5.4 Hz, 1H), 6.57-6.52 (m, 1H), 6.29 (s, 2H), 3.41-3.34 (m, 2H), 2.84 (t, J=6.7 Hz, 2H).

### Example 229. 3-[(3-chloro-2-methoxyphenyl)amino]-2-[6-[(2-methoxyethyl)amino]-1,5-naphthyridin-4-yl]-1H,5H,6H,7H-pyrrolo[3,2-c]pyridin-4-one; formic acid (compound 680)

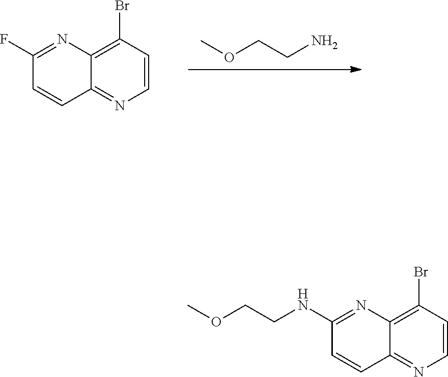

A solution of 8-bromo-2-fluoro-1,5-naphthyridine (100.00 mg, 0.440 mmol, 1.00 equiv) in tert-butanol (4.00 mL) was treated with ethanamine, 2-methoxy- (264.67 mg, 3.528 mmol, 8.00 equiv) under Argon atmosphere. The resulting mixture was stirred for 1.5 hours at 100 degrees C. under Argon atmosphere. Tert-BuOH was removed under reduced pressure and the residue was purified by silica gel column chromatography, eluted with DCM:CH₃₀H=98:2 to afford 8-bromo-N-(2-methoxyethyl)-1,5-naphthyridin-2-amine (110 mg, 88.52%) as a white solid.

LC-MS: (M+H)⁺ found: 282 and 284.

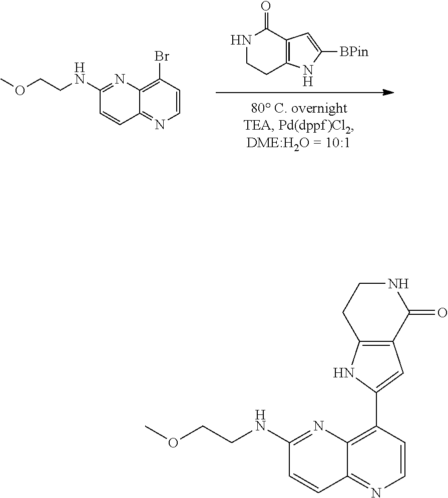

To a solution of 8-bromo-N-(2-methoxyethyl)-1,5-naphthyridin-2-amine (200.00 mg, 0.710 mmol, 1.00 equiv), 2-(4,4,5,5-tetramethyl-1,3,2-dioxaborolan-2-yl)-1H,5H,6H,7H-pyrrolo[3,2-c]pyridin-4-one (460.00 mg, 1.770 mmol, 2.50 equiv) and triethylamine (180.00 mg, 1.770 mmol, 2.50 equiv) in DME:H2O=10:1 was added Pd(dppf)Cl₂CH₂Cl₂ (58.00 mg, 0.070 mmol, 0.10 equiv) inone portion under Argon atmosphere. The resulting mixture was stirred for 10 hours at 80 degrees C. under Argon atmosphere. The mixture was evaporated to remove DME and resolved with CH₃₀H:DCM=1:1. The residue was purified by Prep-TLC (DCM:CH₃₀H=10:1) to afford 2-[6-[(2-methoxyethyl)amino]-1,5-naphthyridin-4-yl]-1H,5H,6H,7H-pyrrolo[3,2-c]pyridin-4-one (210.00 mg, 0.623 mmol, 87.7%) as a brown solid.

LC-MS: (M+H)⁺ found: 338.

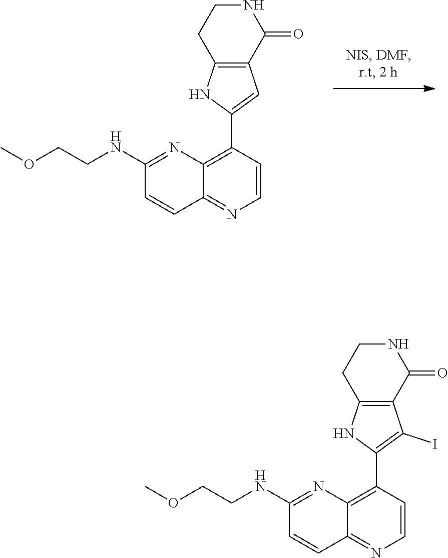 [SMILES: COCCNc1ccc2nccc(-c3cc4c([nH]3)CCNC4=O)c2n1 ; COCCNc1ccc2nccc(-c3[nH]c4c(c3I)C(=O)NCC4)c2n1]

To a stirred solution of 2-[6-[(2-methoxyethyl)amino]-1,5-naphthyridin-4-yl]-1H,5H,6H,7H-pyrrolo[3,2-c]pyridin-4-one (100.00 mg, 0.297 mmol, 1.00 equiv) in DMF (3.00 mL) was added NIS (80.00 mg, 0.356 mmol, 1.20 equiv) dropwise by two times at 0 degrees C. under Argon atmosphere. The resulting mixture was stirred for 2 hours at room temperature under Argon atmosphere. The reaction mixture was then quenched by NaHSO3 (aq) until no more precipitate formed. The precipitate was collected by filtration, washed with excess amount of water and dried under vacuum to afford 3-iodo-2-[6-[(2-methoxyethyl)amino]-1,5-naphthyridin-4-yl]-1H,5H,6H,7H-pyrrolo[3,2-c]pyridin-4-one (120 mg, 87.39%) as a brown solid.

LC-MS: LC-MS: (M+H)⁺ found: 464.

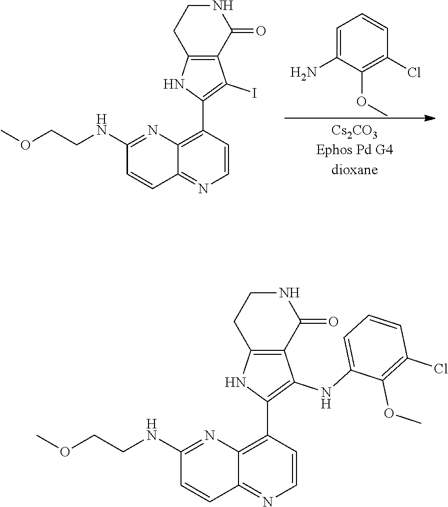

To a solution of 33-iodo-2-[6-[(2-methoxyethyl)amino]-1,5-naphthyridin-4-yl]-1H,5H,6H,7H-pyrrolo[3,2-c]pyridin-4-one (200.00 mg, 0.432 mmol, 1.00 equiv), 3-chloro-2-methoxyaniline (68.04 mg, 0.432 mmol, 1.00 equiv) and Ephos Pd G4 (79.31 mg, 0.086 mmol, 0.20 equiv) in dioxane (4.00 mL) was added cesium carbonate (281.32 mg, 0.864 mmol, 2.00 equiv) in one portion under Argon atmosphere. The resulting mixture was stirred for 10 hours at 50 degrees C. under Argon atmosphere. The resulting mixture was concentrated under reduced pressure. The residue was purified by Prep-TLC DCM:CH3OH=10:1. The crude product was purified by Prep-HPLC(Column: Xselect CSH C18 OBD Column 30*150 mm 5 μm; Mobile Phase A: Water (0.1% FA), Mobile Phase B: ACN; Flow rate: 60 mL/min; Gradient: 20% B to 30% B in 9 min; Wave Length: 254/220 nm; RT1 (min): 8.07) to afford 3-[(3-chloro-2-methoxyphenyl)amino]-2-[6-[(2-methoxyethyl)amino]-1,5-naphthyridin-4-yl]-1H,5H,6H,7H-pyrrolo[3,2-c]pyridin-4-one; formic acid (16 mg, 6.88%) as a orange solid.

LC-MS: LC-MS: (M+H)⁺ found: 540.

¹H NMR (400 MHz, DMSO-d₆) δ 13.07 (s, 1H), 8.41-8.16 (m, 2H), 7.94 (d, J=9.2 Hz, 1H), 7.87-7.78 (m, 1H), 7.74 (s, 1H), 7.38 (d, J=5.0 Hz, 1H), 7.23-7.03 (m, 2H), 6.31-6.02 (m, 1H), 3.90 (s, 3H), 3.66 (d, J=11.8 Hz, 3H), 3.46 (td, J=6.8, 2.5 Hz, 2H), 3.33 (s, 3H), 2.94 (t, J=6.8 Hz, 2H).

### Example 230. 2-[6-(dimethylamino)-1,5-naphthyridin-4-yl]-3-[(3-fluoro-2-methoxyphenyl)amino]-1H,5H,6H,7H-pyrrolo[3,2-c]pyridin-4-one (compound 697)

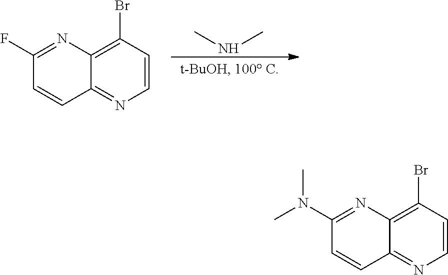

A mixture of 8-bromo-2-fluoro-1,5-naphthyridine (500 mg, 2.20 mmol, 1.00 equiv) and dimethylamine (794 mg, 17.6 mmol, 8.00 equiv) in tert-butanol (20.0 mL) was stirred for 1 h at 100 degrees C. under nitrogen atmosphere. The aqueous layer was extracted with CH₂Cl₂ (3×70 mL). The resulting mixture was concentrated under reduced pressure. This resulted in 8-bromo-N,N-dimethyl-1,5-naphthyridin-2-amine (570 mg, 101.63%) as a yellow solid.

LC-MS: (M+H)⁺ found: 252.0.

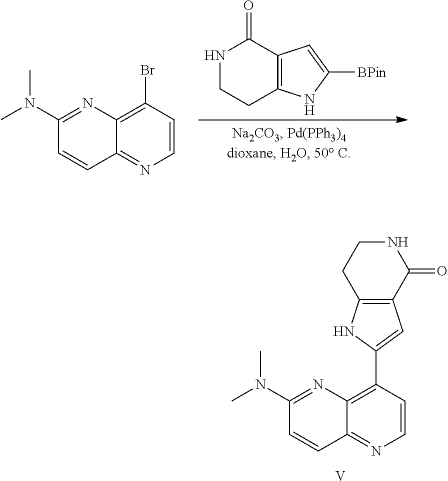

A mixture of 8-bromo-N,N-dimethyl-1,5-naphthyridin-2-amine (520 mg, 2.06 mmol, 1.00 equiv), 2-(4,4,5,5-tetramethyl-1,3,2-dioxaborolan-2-yl)-1H,5H,6H,7H-pyrrolo[3,2-c]pyridin-4-one (810 mg, 3.00 mmol, 1.50 equiv), Na2CO3 (655 mg, 6.18 mmol, 3.00 equiv) and Pd(PPh₃)₄ (476 mg, 0.41 mmol, 0.20 equiv) in dioxane (20.0 mL) and H₂O (4.00 mL) under nitrogen atmosphere. The resulting mixture was stirred for overnight at 50 degrees C. under nitrogen atmosphere. The resulting mixture was filtered, the filter cake was washed with MeOH (2×3 mL). The filtrate was concentrated under reduced pressure. The residue was purified by reverse flash chromatography with the following conditions: column, silica gel; mobile phase, MeCN in water, 10% to 50% gradient in 10 min; detector, UV 254 nm. This resulted in 2-[6-(dimethylamino)-1,5-naphthyridin-4-yl]-1H,5H,6H,7H-pyrrolo[3,2-c]pyridin-4-one (500 mg, 74.93%) as a yellow solid.

LC-MS: (M+H)⁺ found: 308.10.

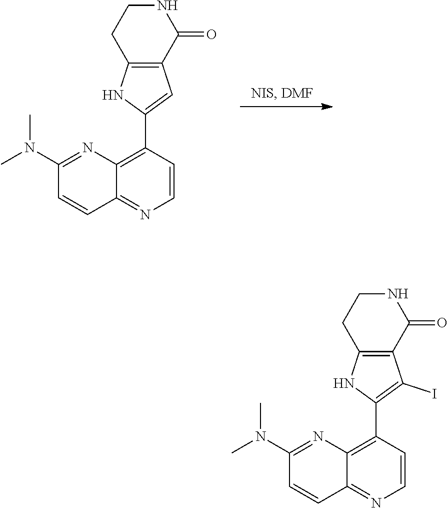 [SMILES: CN(C)c1ccc2nccc(-c3cc4c([nH]3)CCNC4=O)c2n1 ; CN(C)c1ccc2nccc(-c3[nH]c4c(c3I)C(=O)NCC4)c2n1]

A mixture of 2-[6-(dimethylamino)-1,5-naphthyridin-4-yl]-1H,5H,6H,7H-pyrrolo[3,2-c]pyridin-4-one (200 mg, 0.65 mmol, 1.00 equiv) in DMF (20.00 mL) was added NIS (175 mg, 0.78 mmol, 1.20 equiv) in three portions at room temperature under nitrogen atmosphere. The mixture was stirred for 2 h at room temperature under nitrogen atmosphere. The resulting mixture was stirred for additional 1 h at room temperature. The reaction was quenched with sat. Na2SO3 (aq.) at 0 degrees C. The aqueous layer was extracted with CH₂Cl₂ (3×100 mL). The residue was purified by Prep-TLC (PE/EtOAc 1:1) to afford 2-[6-(dimethylamino)-1,5-naphthyridin-4-yl]-3-iodo-1H,5H,6H,7H-pyrrolo[3,2-c]pyridin-4-one (118 mg, 35.12%) as a light yellow solid.

LC-MS: (M+H)⁺ found: 433.95.

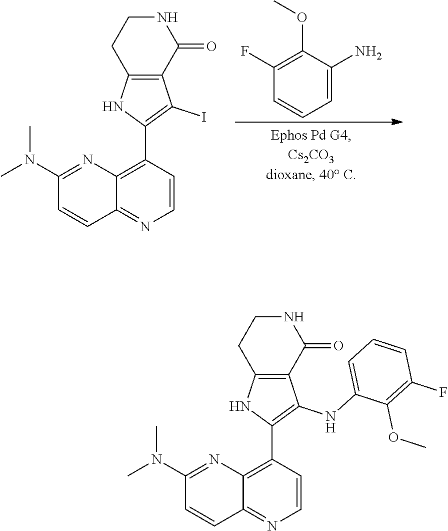

A mixture of 2-[6-(dimethylamino)-1,5-naphthyridin-4-yl]-3-iodo-1H,5H,6H,7H-pyrrolo[3,2-c]pyridin-4-one (100 mg, 0.23 mmol, 1.00 equiv) in dioxane (3.00 mL) under argon atmosphere. To the above mixture was added 3-fluoro-2-methoxyaniline (97 mg, 0.69 mmol, 3.00 equiv), EPhos Pd G4 (42 mg, 0.04 mmol, 0.20 equiv) and Cs₂CO₃ (150 mg, 0.46 mmol, 2.00 equiv) under argon atmosphere. The resulting mixture was stirred for additional overnight at 40 degrees C. The aqueous layer was extracted with CH₂Cl₂ (3×30 mL). The resulting mixture was concentrated under reduced pressure. The residue was purified by Prep-TLC (CH₂Cl₂/MeOH 10:1) to afford crude products. The crude product (60 mg) was purified by Prep-HPLC with the following conditions (Column: XBridge Prep Phenyl OBD Column, 19*250 mm, 5|Ìm; Mobile Phase A: Water (10 mmol/L NH4HCO3), Mobile Phase B: MeOH-Preparative; Flow rate: 25 mL/min; Gradient: 71% B to 87% B in 9 min, 87% B; Wave Length: 254 nm; RT1 (min): 7.97) to afford 2-[6-(dimethylamino)-1,5-naphthyridin-4-yl]-3-[(3-fluoro-2-methoxyphenyl)amino]-1H,5H,6H,7H-pyrrolo[3,2-c]pyridin-4-one (29.0 mg, 26.85%) as a light yellow solid.

LC-MS: (M+H)⁺ found: 447.10.

¹H NMR (300 MHz, DMSO-d6) δ 12.95 (s, 1H), 8.35 (d, J=4.9 Hz, 1H), 8.08 (d, J=9.4 Hz, 1H), 7.76 (s, 1H), 7.47-7.36 (m, 2H), 7.17 (t, J=2.5 Hz, 1H), 6.68 (td, J=8.3, 6.1 Hz, 1H), 6.52 (ddd, J=10.0, 8.4, 1.5 Hz, 1H), 6.18-6.03 (m, 1H), 3.86 (s, 3H), 3.45 (td, J=6.9, 2.4 Hz, 2H), 2.91 (t, J=6.8 Hz, 2H).

### Example 231. 2-(42-aminopyrimidin-4-yl)-3-[[2-methoxy-3-(trifluoromethyl)phenyl]amino]-1H,5H,6H,7H-pyrrolo[3,2-c]pyridin-4-one (compound 698)

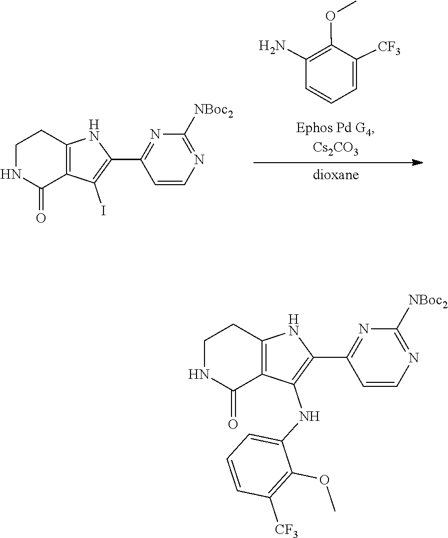

A mixture of tert-butyl tert-butyl N-(tert-butoxycarbonyl)-N-(4-[3-iodo-4-oxo-1H,5H,6H,7H-pyrrolo[3,2-c]pyridin-2-yl]pyrimidin-2-yl)carbamate (120 mg, 0.21 mmol, 1.00 equiv) in dioxane (2.00 mL) under argon atmosphere. To the above mixture was added 2-methoxy-3-(trifluoromethyl) aniline (124 mg, 0.65 mmol, 3.00 equiv), Ephos Pd G4 (40 mg, 0.04 mmol, 0.20 equiv) and Cs₂CO₃ (140 mg, 0.43 mmol, 2.00 equiv) under argon atmosphere. The resulting mixture was stirred for additional overnight at 40 degrees C. The aqueous layer was extracted with EtOAc (3×10 mL). The resulting mixture was concentrated under reduced pressure. The residue was purified by Prep-TLC (PE/EtOAc 1:1) to afford tert-butyl N-(tert-butoxycarbonyl)-N-[4-(3-[[2-methoxy-3-(trifluoromethyl)phenyl]amino]-4-oxo-1H,5H,6H,7H-pyrrolo[3,2-c]pyridin-2-yl)pyrimidin-2-yl]carbamate (63 mg, 16.03%) as a light yellow solid.

LC-MS: (M+H)⁺ found: 619.05.

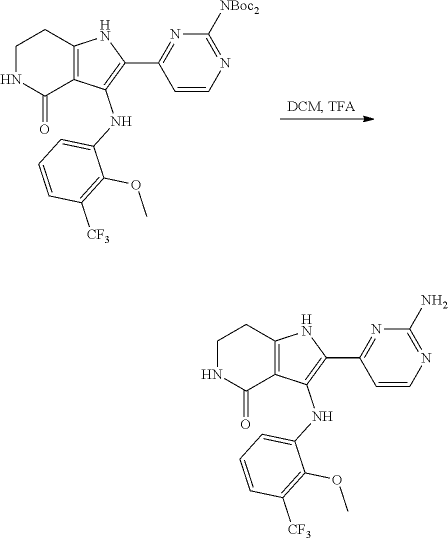

A mixture of tert-butyl tert-butyl N-(tert-butoxycarbonyl)-N-[4-(3-[[2-methoxy-3-(trifluoromethyl)phenyl]amino]-4-oxo-1H,5H,6H,7H-pyrrolo[3,2-c]pyridin-2-yl)pyrimidin-2-yl]carbamate (62 mg, 0.10 mmol, 1.00 equiv) in DCM (1.00 mL). To the above mixture was added TFA (1.00 mL) at 0 degrees C. The resulting mixture was stirred for additional 2 h at room temperature. The resulting mixture was concentrated under reduced pressure. The crude product (60 mg) was purified by Prep-HPLC with the following conditions (Column: XBridge Prep Phenyl OBD Column, 19*250 mm, 5|Ìm; Mobile Phase A: Water (1OMMOL/L NH4HCO3+0.1% NH3·H2O), Mobile Phase B: MeOH-Preparative; Flow rate: 25 mL/min; Gradient: 56% B to 63% B in 10 min, 63% B; Wave Length: 254 nm; RT1 (min): 9.5) to afford 2-(2-aminopyrimidin-4-yl)-3-[[2-methoxy-3-(trifluoromethyl)phenyl]amino]-1H,5H,6H,7H-pyrrolo[3,2-c]pyridin-4-one (38.3 mg, 89.88%) as a white solid.

LC-MS: (M+H)⁺ found: 419.0.

¹H NMR (300 MHz, DMSO-d6) δ 11.70 (s, 1H), 8.18 (s, 1H), 8.08 (d, J=5.4 Hz, 1H), 7.15 (d, J=2.7 Hz, 1H), 7.05-6.89 (m, 2H), 6.75 (dd, J=7.5, 2.3 Hz, 1H), 6.52 (d, J=5.3 Hz, 1H), 6.19 (s, 2H), 3.92 (s, 3H), 3.40 (td, J=6.7, 2.5 Hz, 2H), 2.86 (t, J=6.7 Hz, 2H).

### Example 232. 3-((3-chloro-2-methoxyphenyl)amino)-2-(2-((2-(dimethylamino)ethyl)amino)pyrimidin-4-yl)-1,5,6,7-tetrahydro-4H-pyrrolo[3,2-c]pyridin-4-one (compound 707)

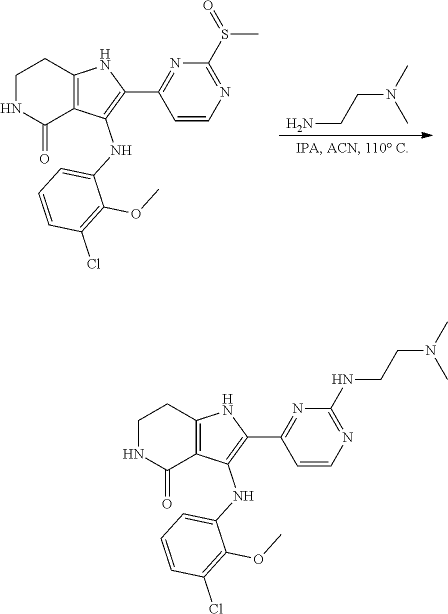

To a stirred mixture of 3-[(3-chloro-2-methoxyphenyl)amino]-2-(2-methanesulfinylpyrimidin-4-yl)-1H,5H,6-H,7H-pyrrolo[3,2-c]pyridin-4-one (80 mg, 0.186 mmol, 1.0 equiv), (2-aminoethyl)dimethylamine (164 mg, 1.86 mmol, 10.0 equiv) in MeOH/ACN (1:1, 2 mL) in portions under nitrogen atmosphere. The resulting mixture was stirred for 2 hours at 110 degrees C. under nitrogen atmosphere. Desired product could be detected by LCMS. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 8% B to 20% B in 8 min, 20% B; Wave Length: 254/220 nm; RT1 (min): 7) to afford 3-((3-chloro-2-methoxyphenyl)amino)-2-(2-((2-(dimethylamino)ethyl)amino)pyrimidin-4-yl)-1,5,6,7-tetrahydro-4H-pyrrolo[3,2-c]pyridin-4-one (5.9 mg, 7.0%) as a yellow solid.

LC-MS: M+H found: 456.10.

¹H NMR (400 MHz, DMSO-d6) δ 11.70 (s, 1H), 8.08 (d, J=5.2 Hz, 1H), 7.91 (s, 1H), 7.16 (d, J=3.0 Hz, 1H), 6.89-6.71 (m, 2H), 6.61-6.32 (m, 3H), 3.89 (s, 3H), 3.57-3.32 (m, 6H), 2.87 (t, J=6.8 Hz, 2H), 2.25 (s, 6H).

### Example 233. 3-((3-chloro-2-methoxyphenyl)amino)-2-(2-((tetrahydro-2H-pyran-4-yl)amino)pyrimidin-4-yl)-1,5,6,7-tetrahydro-4H-pyrrolo[3,2-c]pyridin-4-one (compound 708)

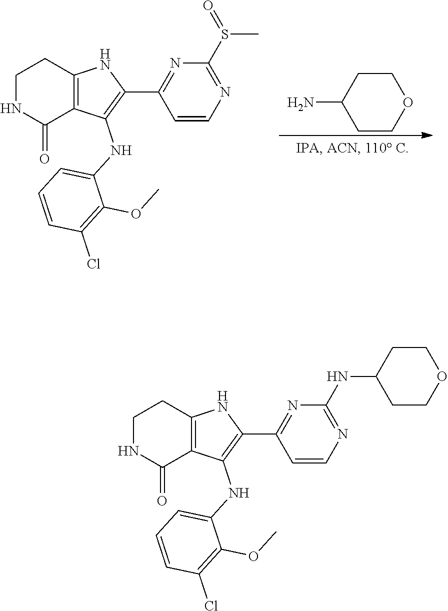 [SMILES: COc1c(Cl)cccc1Nc1c(-c2ccnc(S(C)=O)n2)[nH]c2c1C(=O)NCC2 ; NC1CCOCC1 ; COc1c(Cl)cccc1Nc1c(-c2ccnc(NC3CCOCC3)n2)[nH]c2c1C(=O)NCC2]

To a stirred mixture of 3-[(3-chloro-2-methoxyphenyl)amino]-2-(2-methanesulfinylpyrimidin-4-yl)-1H,5H,6-H,7H-pyrrolo[3,2-c]pyridin-4-one (80 mg, 0.186 mmol, 1.0 equiv), oxan-4-amine (188 mg, 1.86 mmol, 10.0 equiv) in MeOH/ACN (1:1, 2 mL) in portions under nitrogen atmosphere. The resulting mixture was stirred for 2 hours at 110 degrees C. under nitrogen atmosphere. Desired product could be detected by LCMS. The crude product was purified by Prep-HPLC with the following conditions (Column: XBridge BEH C18 OBD Prep Column, 19*250 mm, 5 μm; Mobile Phase A: Water (0.05% FA), Mobile Phase B: ACN; Flow rate: 25 mL/min; Gradient: 22% B to 39% B in 11 min, 39% B; Wave Length: 254/220 nm; RT1 (min): 7) to afford 3-((3-chloro-2-methoxyphenyl)amino)-2-(2-((tetrahydro-2H-pyran-4-yl)amino)pyrimidin-4-yl)-1,5,6,7-tetrahydro-4H-pyrrolo[3,2-c]pyridin-4-one (7.3 mg, 8.3%) as a yellow solid.

LC-MS: M+H found: 469.05.

¹H NMR (400 MHz, DMSO-d6) δ 11.54 (s, 1H), 8.08 (d, J=5.3 Hz, 1H), 7.89 (s, 1H), 7.14 (s, 1H), 6.88-6.71 (m, 3H), 6.52 (d, J=5.3 Hz, 1H), 6.38 (dd, J=7.8, 1.8 Hz, 1H), 4.06-3.94 (m, 1H), 3.89 (s, 3H), 3.81 (s, 2H), 3.49-3.33 (m, 4H), 2.89 (d, J=6.4 Hz, 2H), 1.74 (d, J=12.6 Hz, 2H), 1.55-1.37 (m, 2H).

### Example 234. 3-[(3-chloro-2-methoxyphenyl)amino]-2-[2-[(2-fluoroethyl)amino]pyrimidin-4-yl]-1H,5H,6H,7H-pyrrolo[3,2-c]pyridin-4-one (compound 710)

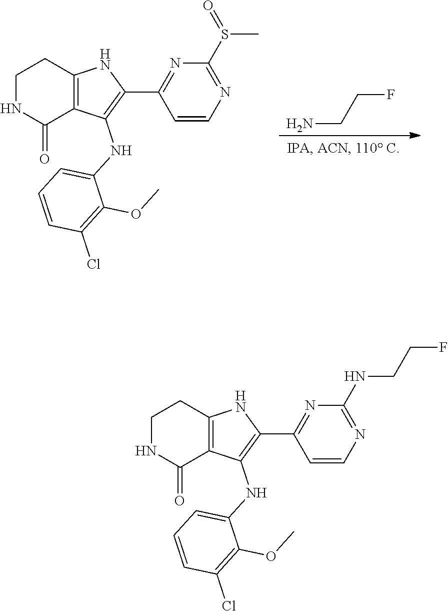

A solution of 3-[(3-chloro-2-methoxyphenyl) amino]-2-(2-methanesulfinylpyrimidin-4-yl)-1H,5H,6H,7H-pyrrolo[3,2-c]pyridin-4-one (80 mg, 0.18 mmol, 1.00 equiv) and 2-fluoroethanamine (116 mg, 1.85 mmol, 10.00 equiv) in ACN (1.00 mL) and IPA (1.00 mL) was stirred for 3 h at 110 degrees C. under nitrogen atmosphere. The aqueous layer was extracted with CH₂Cl₂ (2×20 mL). The resulting mixture was concentrated under reduced pressure. The crude product (80 mg) was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 13% B to 29% B in 8 min, 29% B; Wave Length: 254 nm; RT1 (min): 7.9) to afford 3-[(3-chloro-2-methoxyphenyl)amino]-2-[2-[(2-fluoroethyl)amino]pyrimidin-4-yl]-1H,5H,6H,7H-pyrrolo[3,2-c]pyridin-4-one (8.1 mg, 10.00%) as a light yellow solid LC-MS: M+H found: 431.30.

¹H NMR (300 MHz, DMSO-d6) δ 8.24-7.94 (m, 1H), 6.87-6.70 (m, 2H), 6.48 (d, J=5.4 Hz, 1H), 6.32 (dd, J=7.3, 2.4 Hz, 1H), 4.48 (dt, J=47.7, 5.0 Hz, 2H), 3.62 (dt, J=26.7, 5.0 Hz, 2H), 3.40 (t, J=6.8 Hz, 2H), 2.86 (t, J=6.8 Hz, 2H).

### Example 235. 2-(2-aminopyrimidin-4-yl)-3-[(3-chloro-4-fluoro-2-methoxyphenyl)amino]-1H,5H,6H,7H-pyrrolo[3,2-c]pyridin-4-one (compound 713)

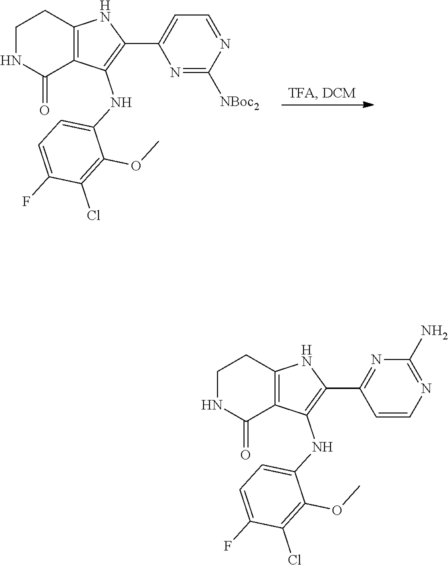

To a stirred mixture of tert-butyl N-(tert-butoxycarbonyl)-N-(4-[3-[(3-chloro-4-fluoro-2-methoxyphenyl)amino]-4-oxo-1H,5H,6H,7H-pyrrolo[3,2-c]pyridin-2-yl]pyrimidin-2-yl)carbamate (99.00 mg, 0.164 mmol, 1.00 equiv) in TFA (1.50 mL) and DCM (1.00 mL) in portions at 0 degrees C. under nitrogen atmosphere. The resulting mixture was stirred for 1 h at room temperature under nitrogen atmosphere. Desired product could be detected by LCMS. The crude product (100 mg) was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 11% B to 27% B in 8 min, 27% B; Wave Length: 254/220 nm; RT1 (min): 7.23) to afford 2-(2-aminopyrimidin-4-yl)-3-[(3-chloro-4-fluoro-2-methoxyphenyl)amino]-1H,5H,6H,7H-pyrrolo[3,2-c]pyridin-4-one (18.1 mg, 26.63%) as a yellow solid.

LC-MS: M+H found: 403.

¹H NMR (400 MHz, DMSO-d6) δ 11.78 (s, 1H), 8.23 (s, 1H), 8.17-8.00 (m, 1H), 7.13 (d, J=2.8 Hz, 1H), 6.94 (t, J=9.0 Hz, 1H), 6.83-6.58 (m, 3H), 6.51 (dd, J=9.2, 5.6 Hz, 1H), 3.91 (s, 3H), 3.40 (dd, J=6.6, 2.6 Hz, 2H), 2.86 (d, J=13.4 Hz, 2H).

### Example 236. 3-[(3-fluoro-2-methoxyphenyl)amino]-2-{2-[(2-methyl-1,2,3-triazol-4-yl)amino]pyrimidin-4-yl}-1H,5H,6H,7H-pyrrolo[3,2-c]pyridin-4-one (compound 593)

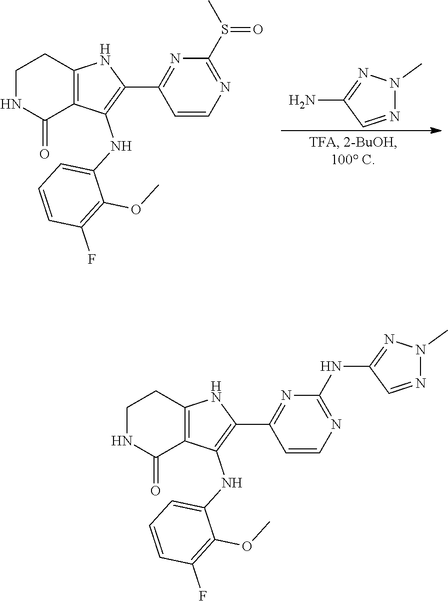

To a stirred solution/mixture of 3-[(3-fluoro-2-methoxyphenyl)amino]-2-(2-methanesulfinylpyrimidin-4-yl)-1H,5H,6H,7H-pyrrolo[3,2-c]pyridin-4-one (100 mg, 0.241 mmol, 1 equiv) and 2-methyl-1,2,3-triazol-4-amine (47.23 mg, 0.482 mmol, 2 equiv) in 2-methyl-2-butanol (1 mL) was added TFA (54.89 mg, 0.482 mmol, 2 equiv) dropwise at r.t. The solution was stirred at 100° C. for overnight. LCMS showed ok. The resulting mixture was concentrated under reduced pressure. The mixture/residue was neutralized to pH=10 with saturate NaHCO3. The precipitated solids were collected by filtration and washed with water (3×3 mL) amd purified with Column: XBridge Prep C18 OBD Column, 30*100 mm, 5pim; Mobile Phase A: Water (10 mmol/L NH4HCO3), Mobile Phase B: ACN; Flow rate: 60 mL/min; Gradient: 20% B to 42% B in 9 min, 42% B; Wave Length: 254/220 nm; RT1 (min): 9.67 to give 3-[(3-fluoro-2-methoxyphenyl)amino]-2-{2-[(2-methyl-1,2,3-triazol-4-yl)amino]pyrimidin-4-yl}-1H,5H,6H,7H-pyrrolo[3,2-c]pyridin-4-one (10.9 mg, 10.08%) as light-yellow solid.

LC-MS: (M+H)⁺ found: 450.05.

¹H NMR (400 MHz, DMSO-d6) δ 11.45 (s, 1H), 9.80 (s, 1H), 8.25 (d, J=5.3 Hz, 1H), 8.11 (s, 1H), 7.78 (s, 1H), 7.21 (t, J=2.6 Hz, 1H), 6.78-6.73 (m, 1H), 6.71 (d, J=5.4 Hz, 1H), 6.60-6.54 (m, 1H), 6.16 (d, J=8.3 Hz, 1H), 4.05 (s, 3H), 3.93 (d, J=0.9 Hz, 3H), 3.41 (d, J=2.5 Hz, 2H), 2.91 (t, J=6.8 Hz, 2H).

### Example 237. 3-[(3-chloro-2-methoxyphenyl)amino]-2-{2-[(2-methyl-1,2,3-triazol-4-yl)amino]pyrimidin-4-yl}-1H,5H,6H,7H-pyrrolo[3,2-c]pyridin-4-one (compound 592)

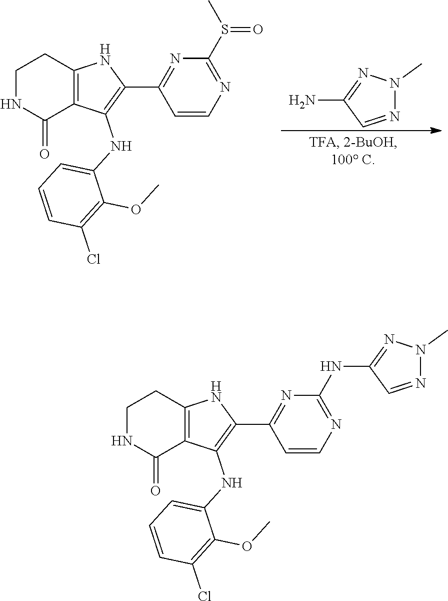

To a stirred solution/mixture of 3-[(3-chloro-2-methoxyphenyl)amino]-2-(2-methanesulfinylpyrimidin-4-yl)-1H,5H,6H,7H-pyrrolo[3,2-c]pyridin-4-one (100 mg, 0.232 mmol, 1 equiv) and 2-methyl-1,2,3-triazol-4-amine (45.43 mg, 0.464 mmol, 2 equiv) in 2-methyl-2-butanol (1 mL) was added TFA (52.80 mg, 0.464 mmol, 2 equiv) dropwise at r.t. The solution was stirred at 100° C. for overnight. LCMS showed ok. The resulting mixture was concentrated under reduced pressure. The mixture/residue was neutralized to pH=10 with saturate NaHCO₃. The precipitated solids were collected by filtration and washed with water (3×3 mL) amd purified with Column: Xselect CSH C18 OBD Column 30*150 mm 5 μm, n; Mobile Phase A: Water (0.05% TFA), Mobile Phase B: ACN; Flow rate: 60 mL/min; Gradient: 24% B to 42% B in 8 min, 42% B; Wave Length: 254/220 nm; RT1 (min): 8 to give 3-[(3-chloro-2-methoxyphenyl)amino]-2-{2-[(2-methyl-1,2,3-triazol-4-yl)amino]pyrimidin-4-yl}-1H,5H,6H,7H-pyrrolo[3,2-c]pyridin-4-one (42.6 mg, 39.49%) as yellow solid.

LC-MS: (M+H)⁺ found: 466.05.

¹H NMR (400 MHz, DMSO-d₆) δ 11.57 (s, 1H), 10.01 (s, 1H), 8.24 (d, J=5.5 Hz, 1H), 8.08 (s, 1H), 7.90 (s, 1H), 7.23 (d, J=5.3 Hz, 1H), 6.81-6.78 (m, 2H), 6.73 (d, J=5.6 Hz, 1H), 6.37-6.33 (m, 1H), 4.05 (s, 3H), 3.88 (s, 3H), 3.42 (t, J=6.9 Hz, 2H), 2.92 (t, J=6.7 Hz, 2H).

### Example 238. 3-[(3-fluoro-2-methoxyphenyl)amino]-2-{2-[(3-methyl-1,2,3-triazol-4-yl)amino]pyrimidin-4-yl}-1H,5H,6H,7H-pyrrolo[3,2-c]pyridin-4-one (compound 591)

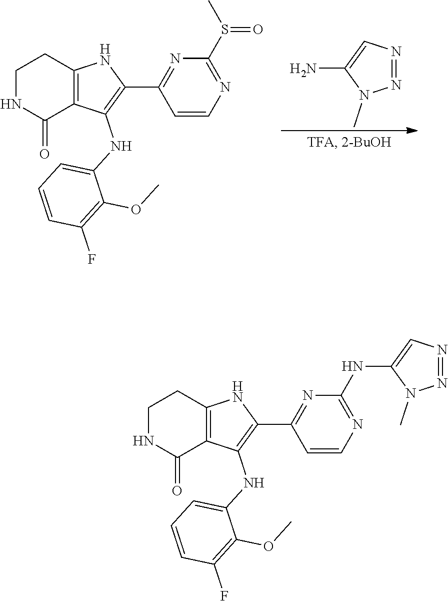

To a stirred solution/mixture of 3-[(3-fluoro-2-methoxyphenyl)amino]-2-(2-methanesulfinylpyrimidin-4-yl)-1H,5H,6H,7H-pyrrolo[3,2-c]pyridin-4-one (100 mg, 0.241 mmol, 1 equiv) and 3-methyl-1,2,3-triazol-4-amine (47.23 mg, 0.482 mmol, 2 equiv) in 2-methyl-2-butanol (1 mL) was added TFA (54.89 mg, 0.482 mmol, 2 equiv) dropwise at r.t. The solution was stirred at 100° C. for overnight. LCMS showed ok. The resulting mixture was concentrated under reduced pressure. The mixture/residue was neutralized to pH=10 with saturate NaHCO₃. The precipitated solids were collected by filtration and washed with water (3×3 mL) amd purified with Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 25% B to 44% B in 9 min, 44% B; Wave Length: 254/220 nm; RT1 (min): 8.85 to give 3-[(3-fluoro-2-methoxyphenyl)amino]-2-{2-[(3-methyl-1,2,3-triazol-4-yl)amino]pyrimidin-4-yl}-1H,5H,6H,7H-pyrrolo[3,2-c]pyridin-4-one (21.2 mg, 19.03%) as light-yellow solid.

LC-MS: (M+H)⁺ found: 450.05.

¹H NMR (400 MHz, DMSO-d6) δ 11.48 (s, 1H), 9.80 (s, 1H), 8.25 (d, J=5.3 Hz, 1H), 8.11 (s, 1H), 7.77 (s, 1H), 7.20 (t, J=2.6 Hz, 1H), 6.79-6.66 (m, 2H), 6.63-6.53 (m, 1H), 4.05 (s, 3H), 3.98-3.84 (m, 3H), 3.43-3.39 (m, 2H), 2.91 (t, J=6.8 Hz, 2H).

### Example 239. 2-{2-[(1,5-dimethyl-1,2,3-triazol-4-yl)amino]pyrimidin-4-yl}-3-[(3-fluoro-2-methoxyphenyl)amino]-1H,5H,6H,7H-pyrrolo[3,2-c]pyridin-4-one (compound 608)

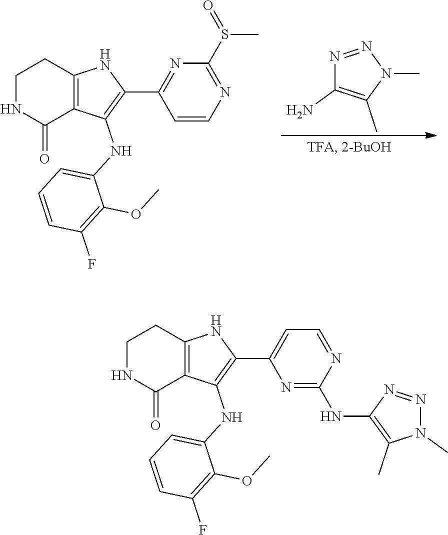

To a stirred solution/mixture of 3-[(3-fluoro-2-methoxyphenyl)amino]-2-(2-methanesulfinylpyrimidin-4-yl)-1H,5H,6H,7H-pyrrolo[3,2-c]pyridin-4-one (100 mg, 0.241 mmol, 1 equiv) and dimethyl-1,2,3-triazol-4-amine (53.98 mg, 0.482 mmol, 2 equiv) in 2-methyl-2-butanol (1 mL) was added TFA (54.89 mg, 0.482 mmol, 2 equiv) dropwise at r.t. The solution was stirred at 100° C. for overnight. LCMS showed ok. The resulting mixture was concentrated under reduced pressure. The mixture/residue was neutralized to pH=10 with saturate NaHCO₃. The precipitated solids were collected by filtration and washed with water (3×3 mL) amd purified with Column: YMC-Actus Triart C18 ExRS, 30*150 mm, 5 μm; Mobile Phase A: Water (10 mmol/L NH4HCO3), Mobile Phase B: ACN; Flow rate: 60 mL/min; Gradient: 18% B to 35% B in 10 min, 35% B; Wave Length: 254/220 nm; RT1 (min): 8.85 to give 2-{2-[(1,5-dimethyl-1,2,3-triazol-4-yl)amino]pyrimidin-4-yl}-3-[(3-fluoro-2-methoxyphenyl)amino]-1H,5H,6H,7H-pyrrolo[3,2-c]pyridin-4-one (51.6 mg, 46.25%) as light-yellow solid.

LC-MS: (M+H)⁺ found: 464.10.

¹H NMR (400 MHz, DMSO-d6) δ 11.46 (s, 1H), 8.67 (s, 1H), 8.11 (d, J=5.4 Hz, 1H), 7.71 (s, 1H), 7.14 (d, J=2.6 Hz, 1H), 6.81-6.751 (m, 1H), 6.66-6.50 (m, 2H), 6.22-6.19 (m, 1H), 3.90 (d, J=10.0 Hz, 6H), 3.51 (s, 2H), 2.85 (t, J=6.7 Hz, 2H), 2.09 (s, 3H).

### Example 240. 3-[(3-chloro-2-methoxyphenyl)amino]-2-{2-[(1,5-dimethyl-1,2,3-triazol-4-yl)amino]pyrimidin-4-yl}-1H,5H,6H,7H-pyrrolo[3,2-c]pyridin-4-one (compound 607)

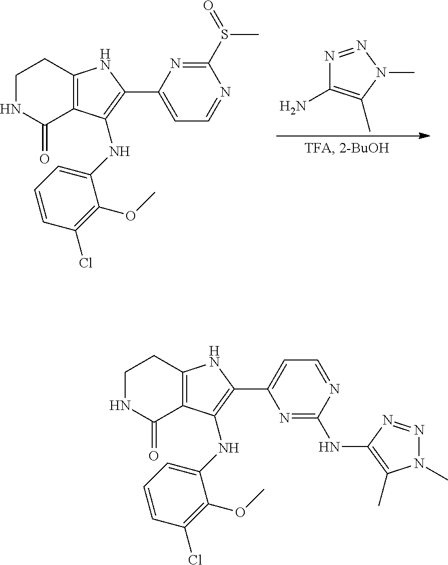

To a stirred solution/mixture of 3-[(3-chloro-2-methoxyphenyl)amino]-2-(2-methanesulfinylpyrimidin-4-yl)-1H,5H,6H,7H-pyrrolo[3,2-c]pyridin-4-one (100 mg, 0.232 mmol, 1 equiv) and dimethyl-1,2,3-triazol-4-amine (51.93 mg, 0.464 mmol, 2 equiv) in 2-methyl-2-butanol (1 mL) was added TFA (52.80 mg, 0.464 mmol, 2 equiv) dropwise at r.t. The solution was stirred at 100° C. for overnight. The resulting mixture was concentrated under reduced pressure. The mixture/residue was neutralized to pH=10 with saturated NaHCO₃. The precipitated solids were collected by filtration and washed with water (3×3 mL) amd purified with Column: Xselect CSH C18 OBD Column 30*150 mm 5 μm, n; Mobile Phase A: Water (0.1% FA), Mobile Phase B: ACN; Flow rate: 60 mL/min; Gradient: 14% B to 32% B in 8 min, 32% B; Wave Length: 254/220 nm; RT1 (min): 8 to give 3-[(3-chloro-2-methoxyphenyl)amino]-2-{2-[(1,5-dimethyl-1,2,3-triazol-4-yl)amino]pyrimidin-4-yl}-1H,5H,6H,7H-pyrrolo[3,2-c]pyridin-4-one (34.6 mg, 31.14%) as yellow solid.

LC-MS: (M+H)⁺ found: 480.10

¹H NMR (400 MHz, DMSO-d₆) δ 11.48 (s, 1H), 8.67 (s, 1H), 8.13 (d, J=5.4 Hz, 1H), 7.73 (s, J H), 7.12 (t, J=2.6 Hz, J H), 6.92-6.72 (m, 2H), 6.63 (d, J=5.4 Hz, 1H), 6.38-6.30 (m, 1H), 3.92 (s, 3H), 3.84 (s, 3H), 3.43-3.38 (m, 2H), 2.85 (t, J=6.7 Hz, 2H), 2.09 (s, 3H).

### Example 241. 3-[(3-chloro-2-methoxyphenyl)amino]-2-{6-[2-(dimethylamino)ethoxy]-7-methoxy-1,5-naphthyridin-4-yl}-1H,5H,6H,7H-pyrrolo[3,2-c]pyridin-4-one (compound 501)

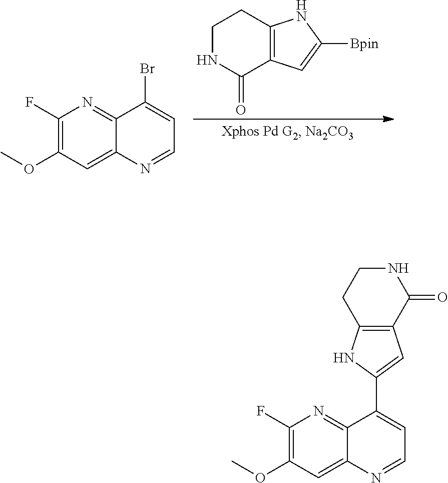

To a stirred mixture of 8-bromo-2-fluoro-3-methoxy-1,5-naphthyridine (300 mg, 1.167 mmol, 1 equiv) and 2-(4,4,5,5-tetramethyl-1,3,2-dioxaborolan-2-yl)-1H,5H,6H,7H-pyrrolo[3,2-c]pyridin-4-one (367.08 mg, 1.400 mmol, 1.2 equiv) in 1,4-dioxane (12 mL) and water (3 mL) were added Na2CO3 (247.38 mg, 2.334 mmol, 2 equiv) and XPhos palladium(II) biphenyl-2-amine chloride (91.82 mg, 0.117 mmol, 0.1 equiv) in portions at room temperature under argon atmosphere. The resulting mixture was stirred for overnight at 50° C. under argon atmosphere. Desired product could be detected by LCMS. The resulting mixture was concentrated under reduced pressure. The residue was purified by silica gel column chromatography, eluted with CH2Cl2/MeOH (20:1) to afford 2-(6-fluoro-7-methoxy-1,5-naphthyridin-4-yl)-1H,5H,6H,7H-pyrrolo[3,2-c]pyridin-4-one (267 mg, 73.26%) as a yellow solid.

LC-MS: [M−H]⁻ found: 313.00.

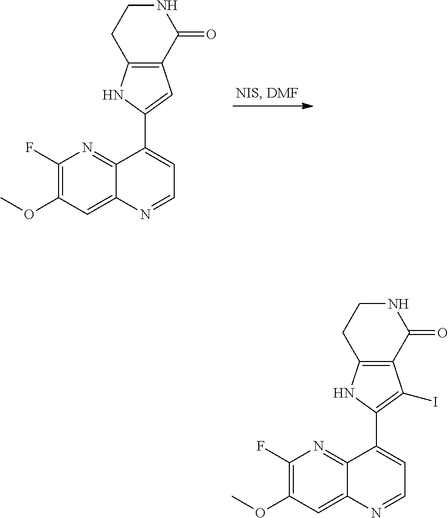 [SMILES: COc1cc2nccc(-c3cc4c([nH]3)CCNC4=O)c2nc1F ; COc1cc2nccc(-c3[nH]c4c(c3I)C(=O)NCC4)c2nc1F]

A mixture of 2-(6-fluoro-7-methoxy-1,5-naphthyridin-4-yl)-1H,5H,6H,7H-pyrrolo[3,2-c]pyridin-4-one (185 mg, 0.592 mmol, 1 equiv) and NIS (199.91 mg, 0.888 mmol, 1.5 equiv) in DMF (6.5 mL) was stirred for overnight at room temperature under air atmosphere. Desired product could be detected by LCMS. The reaction was quenched by the addition of sat. sodium sulfite (aq.) (1 mL) at room temperature. The resulting mixture was concentrated under vacuum. The residue was purified by silica gel column chromatography, eluted with CH₂Cl₂/MeOH (20:1) to afford 2-(6-fluoro-7-methoxy-1,5-naphthyridin-4-yl)-3-iodo-1H,5H,6H,7H-pyrrolo[3,2-c]pyridin-4-one (230 mg, 88.61%) as a yellow solid.

LC-MS: [M−H]⁻ found: 438.85.

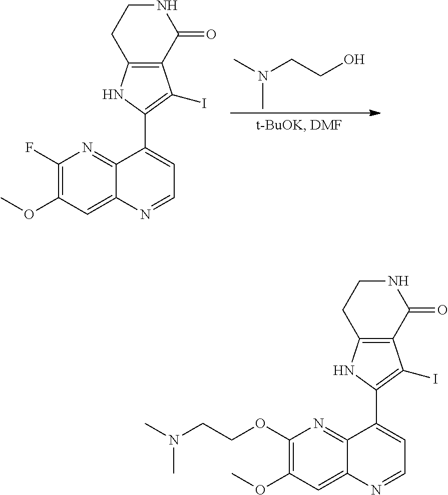

To a stirred mixture of 2-(6-fluoro-7-methoxy-1,5-naphthyridin-4-yl)-3-iodo-1H,5H,6H,7H-pyrrolo[3,2-c]pyridin-4-one (60 mg, 0.137 mmol, 1 equiv) and dimethylaminoethanol (36.62 mg, 0.411 mmol, 3 equiv) in DMF (1 mL) was added Potassium tert-butoxide in THF (I M) (0.16 mL, 0.164 mmol, 1.2 equiv) in portions at 0° C. under argon atmosphere. The resulting mixture was stirred for 1 h at 0° C. under argon atmosphere. Desired product could be detected by LCMS. The reaction was quenched by the addition of Water (1 mL) at 0° C. The resulting mixture was concentrated under reduced pressure. The residue was purified by silica gel column chromatography, eluted with CH2Cl2/MeOH (20:1) to afford 2-{6-[2-(dimethylamino)ethoxy]-7-methoxy-1,5-naphthyridin-4-yl}-3-iodo-1H,5H,6H,7H-pyrrolo[3,2-c]pyridin-4-one (65 mg, 93.57%) as a yellow solid.

LC-MS: [M+H]⁺ found: 508.10.

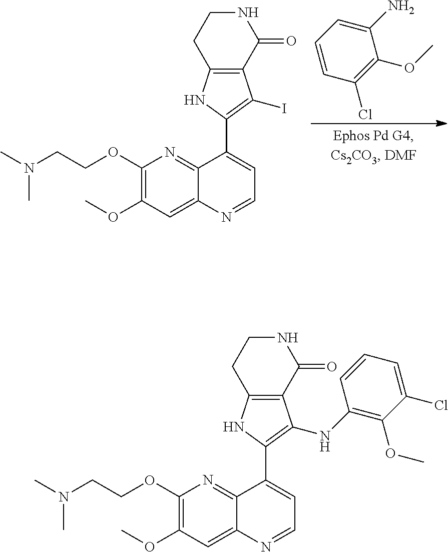

To a stirred mixture of 2-{6-[2-(dimethylamino)ethoxy]-7-methoxy-1,5-naphthyridin-4-yl}-3-iodo-1H,5H,6H,7H-pyrrolo[3,2-c]pyridin-4-one (70 mg, 0.138 mmol, 1.00 equiv) and 3-chloro-2-methoxyaniline (19.57 mg, 0.124 mmol, 0.9 equiv) in DMF (2 mL) were added Cs₂CO₃ (89.91 mg, 0.276 mmol, 2 equiv) and EPhos Pd G4 (12.67 mg, 0.014 mmol, 0.1 equiv) in portions at room temperature under argon atmosphere. The resulting mixture was stirred for 2 h at 50° C. under argon atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 38% B to 54% B in 8 min, 54% B; Wave Length: 254/220 nm; RT1 (min): 7) to afford 3-[(3-chloro-2-methoxyphenyl)amino]-2-{6-[2-(dimethylamino)ethoxy]-7-methoxy-1,5-naphthyridin-4-yl}-1H,5H,6H,7H-pyrrolo[3,2-c]pyridin-4-one (5.7 mg, 7.32%) as a yellow solid.

LC-MS: [M+H]⁺ found: 537.00.

¹H NMR (300 MHz, DMSO-d6) δ 11.90 (s, 1H), 8.49 (d, J=5.1 Hz, 1H), 7.66 (d, J=3.0 Hz, 2H), 7.40 (d, J=4.8 Hz, 1H), 7.17 (s, 1H), 6.65-6.62 (m, 2H), 6.18-6.15 (m, 1H), 4.68 (t, J=6.3 Hz, 2H), 3.99 (s, 3H), 3.85 (s, 3H), 2.92 (t, J=6.8 Hz, 2H), 2.76 (s, OH), 2.24 (s, 6H).

### Example 242. 3-[(3-chloro-2-methoxyphenyl)amino]-2-{2-[2-(morpholin-4-yl)ethoxy]pyrido[3,2-d]pyrimidin-8-yl}-1H,5H,6H,7H-pyrrolo[3,2-c]pyridin-4-one (compound 506)

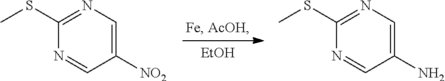

To a stirred solution of 2-(methylsulfanyl)-5-nitropyrimidine (5 g, 29.211 mmol, 1.00 equiv) in EtOH (200 mL) was added AcOH (120 mL) and Fe (17 g, 292.11 mmol, 10 equiv) in portions at rt under N₂ atmosphere. The resulting mixture was stirred for 2 h at 80 degrees C. under N₂ atmosphere. Desired product could be detected by LCMS. LC-MS: M+H found: 142. The resulting mixture was concentrated under reduced pressure. The resulting mixture was extracted with EA (3×100 mL). The combined organic layers were washed with Saturated NaCl (3×100 mL), dried over anhydrous Na₂SO₄. After filtration, the filtrate was concentrated under reduced pressure to afford 2-(methylsulfanyl)pyrimidin-5-amine (3.5 g, 84.86%) as a yellow solid.

LC-MS: M+H found: 142.0.

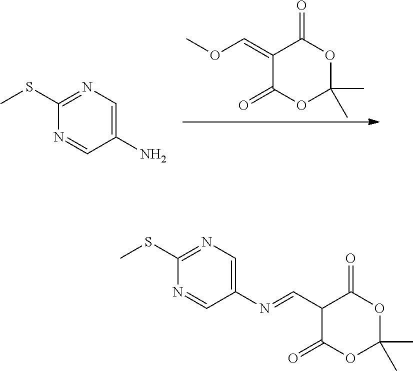

To a stirred solution of 2-(methylsulfanyl)pyrimidin-5-amine (3.2 g, 22.66 mmol, 1.00 equiv) and 5-(methoxymethylidene)-2,2-dimethyl-1,3-dioxane-4,6-dione (5.06 g, 27.18 mmol, 1.20 equiv) in DMF (80.00 mL) at rt under N₂ atmosphere. The resulting mixture was stirred for 1 h at 80 degrees C. under N₂ atmosphere. Desired product could be detected by LCMS. LC-MS: M+H found: 296. The resulting mixture was added MeOH (50 mL). The resulting mixture was filtered, the filter cake was washed with MeOH (3×20 mL). The filter cake was concentrated under reduced pressure to afford 2,2-dimethyl-5-[(1E)-[[2-(methylsulfanyl)pyrimidin-5-yl]imino]methyl]-1,3-dioxane-4,6-dione (5.4 g, 80.68%) as a yellow solid.

LC-MS: (M+H)⁺ found: 296.0.

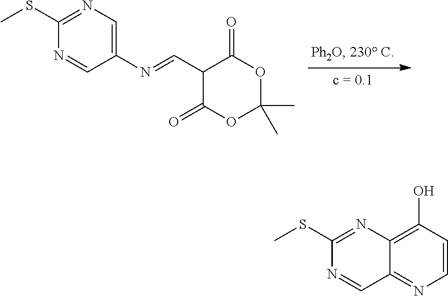

To a stirred solution of 2,2-dimethyl-5-[(1E)-([2-(methylsulfanyl)pyrimidin-5-yl]imino}methyl]-1,3-dioxane-4,6-dione (5.3 g, 17.95 mmol, 1.00 equiv) in phenoxybenzene (360 mL) at rt under N2 atmosphere. The resulting mixture was stirred at 230 degrees C. under N2 atmosphere. Desired product could be detected by LCMS. LC-MS: M+H found: 194. The reaction was addition of Hexane (700 ml) at rt. The resulting mixture was filtered: the filter cake was washed with Hexane (3×200 ml). The filtrate was concentrated under reduced pressure.

LC-MS: (M+H)⁺ found: 194.0.

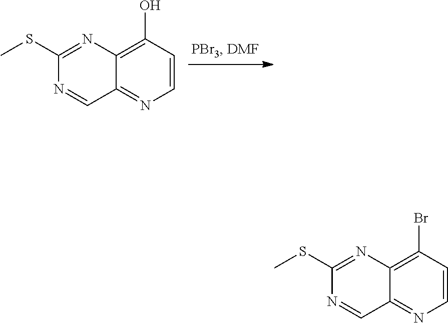

To a stirred solution of 2-(methylsulfanyl)pyrido[3,2-d]pyrimidin-8-ol (2.8 g, 14.49 mmol, 1.00 equiv) in DMF (80 mL) was added PBr3 (4.31 g, 15.94 mmol, 1.1 equiv) dropwise at 0 degrees C. under N₂ atmosphere. The resulting mixture was stirred for 1 h at rt under N₂ atmosphere. Desired product could be detected by LCMS. LC-MS: M+H found: 256. The resulting mixture was extracted with EA (3×100 mL). The combined organic layers were washed with aq. NaCl (3×100 mL), dried over anhydrous Na₂SO₄. After filtration, the filtrate was concentrated under reduced pressure. The residue was purified by silica gel column chromatography, eluted with PE:EA (4:1) to afford 8-bromo-2-(methylsulfanyl)pyrido[3,2-d]pyrimidine (1.6 g, 43.11%) as a white solid.

LC-MS: (M+H)⁺ found: 256.0.

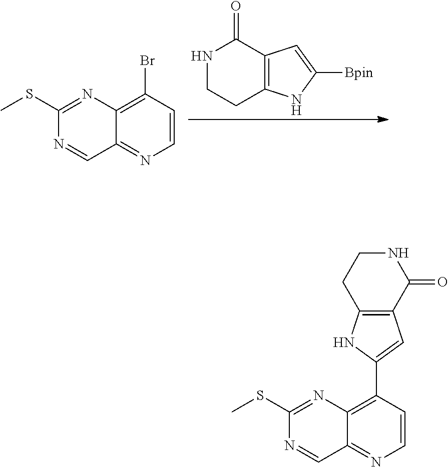

To a stirred solution of 8-bromo-2-(methylsulfanyl)pyrido[3,2-d]pyrimidine (700 mg, 2.73 mmol, 1.00 equiv) and 2-(4,4,5,5-tetramethyl-1,3,2-dioxaborolan-2-yl)-1H,5H,6H,7H-pyrrolo[3,2-c]pyridin-4-one (1075 mg, 4.10 mmol, 1.50 equiv) in dioxane (10 mL) and H₂O (1 mL) were added Na2CO3 (869.03 mg, 8.199 mmol, 3.00 equiv) and XPhos Pd G2 (215 mg, 0.27 mmol, 0.10 equiv) dropwise/in portions at rt under N2 atmosphere. The resulting mixture was stirred for 2 h at 80 degrees C. under N2 atmosphere. Desired product could be detected by LCMS. LC-MS: M+H found: 312. The resulting mixture was concentrated under vacuum. The residue was purified by silica gel column chromatography, eluted with DCM/MeOH=24:1 to afford 2-[2-(methylsulfanyl)pyrido[3,2-d]pyrimidin-8-yl]-1H,5H,6H,7H-pyrrolo[3,2-c]pyridin-4-one (700 mg, 82.26%) as a yellow solid.

LC-MS: (M+H)⁺ found: 312.0.

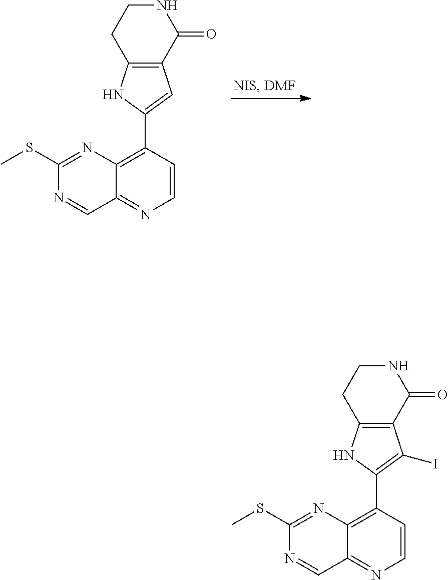 [SMILES: CSc1ncc2nccc(-c3cc4c([nH]3)CCNC4=O)c2n1 ; CSc1ncc2nccc(-c3[nH]c4c(c3I)C(=O)NCC4)c2n1]

To a stirred solution of 2-[2-(methylsulfanyl)pyrido[3,2-d]pyrimidin-8-yl]-1H,5H,6H,7H-pyrrolo[3,2-c]pyridin-4-one (600 mg, 1.93 mmol, 1.00 equiv) and NIS (650.33 mg, 2.891 mmol, 1.50 equiv) in DMF (10 mL) at rt under N₂ atmosphere. The resulting mixture was stirred for overnight at 30 degrees C. under N₂ atmosphere. Desired product could be detected by LCMS. LC-MS: M+H found: 438. The reaction was quenched by the addition of Saturated aq. Na2SO3 (20 mL) at 0 degrees C. The precipitated solids were collected by filtration and washed with H₂O (20 mL×3). The residue was purified by silica gel column chromatography, eluted with DCM:MeOH=10:1 to afford 3-iodo-2-[2-(methylsulfanyl)pyrido[3,2-d]pyrimidin-8-yl]-1H,5H,6H,7H-pyrrolo[3,2-c]pyridin-4-one (650 mg, 77.14%) as a yellow solid.

LC-MS: (M+H found: 438.0.

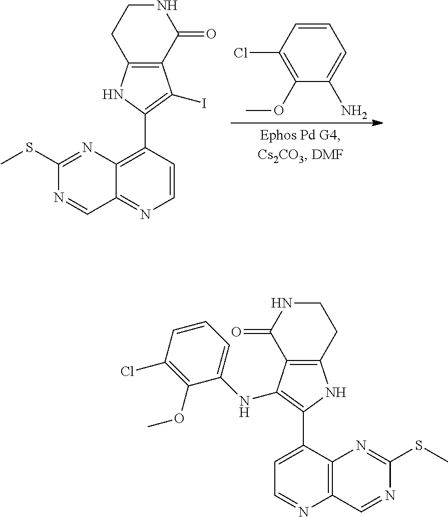

To a stirred solution of 3-iodo-2-[2-(methylsulfanyl)pyrido[3,2-d]pyrimidin-8-yl]-1H,5H,6H,7H-pyrrolo[3,2-c]pyridin-4-one (370 mg, 0.85 mmol, 1.00 equiv) and 3-chloro-2-methoxyaniline (134 mg, 0.85 mmol, 1 equiv) in DMF (4 mL) were added EPhos Pd G4 (78 mg, 0.08 mmol, 0.1 equiv) and Cs₂CO₃ (827 mg, 2.54 mmol, 3 equiv) in portions at rt under Ar atmosphere. The resulting mixture was stirred for 1.5 h at 50 degrees C. under Ar atmosphere. Desired product could be detected by LCMS. LC-MS: M+H found: 467. The resulting mixture was concentrated under vacuum. The residue was purified by silica gel column chromatography, eluted with DCM:MeOH (10:1) to afford 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (220 mg, 55.68%) as a orange solid.

LC-MS: (M+H)⁺ found: 467.0.

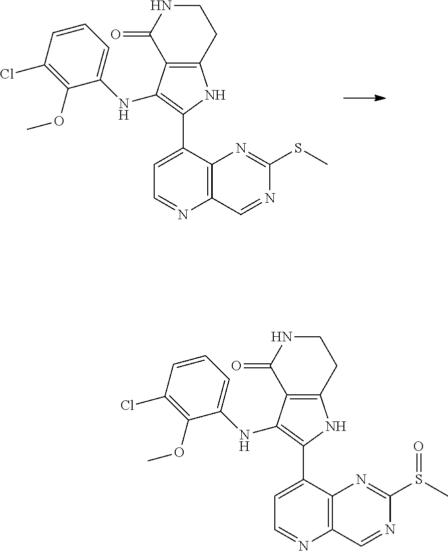 [SMILES: COc1c(Cl)cccc1Nc1c(-c2ccnc3cnc(SC)nc23)[nH]c2c1C(=O)NCC2 ; COc1c(Cl)cccc1Nc1c(-c2ccnc3cnc(S(C)=O)nc23)[nH]c2c1C(=O)NCC2]

To a stirred solution of 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (50 mg, 0.11 mmol, 1.00 equiv) in DCM (2 mL, 31.46 mmol, 293.80 equiv) were added MCPBA (29 mg, 0.12 mmol, 1.1 equiv) dropwise/in portions at 0 degrees C. under N2 atmosphere. The resulting mixture was stirred for 1 h at 0 degrees C. under N2 atmosphere.

Desired product could be detected by LCMS. LC-MS: M+H found: 483. The resulting mixture was extracted with DCM (3×4 mL). The combined organic layers were dried over anhydrous Na₂SO₄. After filtration, the filtrate was concentrated under reduced pressure. The crude product was used in the next step directly without further purification.

LC-MS: (M+H)⁺ found: 483.0.

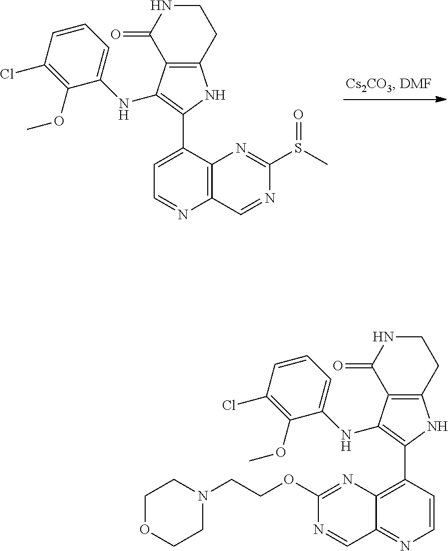 [SMILES: COc1c(Cl)cccc1Nc1c(-c2ccnc3cnc(S(C)=O)nc23)[nH]c2c1C(=O)NCC2 ; COc1c(Cl)cccc1Nc1c(-c2ccnc3cnc(OCCN4CCOCC4)nc23)[nH]c2c1C(=O)NCC2]

To a stirred solution of 3-[(3-chloro-2-methoxyphenyl)amino]-2-{2-methanesulfinylpyrido[3,2-d]pyrimidin-8-yl}-1H,5H,6H,7H-pyrolo[3,2-c]pyridin-4-one (145 mg, 0.300 mmol, 1.00 equiv) and Cs₂CO₃ (234.78 mg, 0.72 mmol, 2.4 equiv) in DMF (5 mL) were added 4-morpholineethanol (47 mg, 0.36 mmol, 1.2 equiv) dropwise at 0 degrees C. under N₂ atmosphere. The resulting mixture was stirred for 1 h at 0 degrees C. under N₂ atmosphere. The resulting mixture was concentrated under vacuum. The resulting mixture was concentrated under vacuum and dissolved in DMF. The crude product (100 mg) was purified by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 22% B to 43% B in 9 min, 43% B; Wave Length: 254/220 nm; RT1 (min): 8.85) to afford 3-[(3-chloro-2-methoxyphenyl)amino]-2-{2-[2-(morpholin-4-yl)ethoxy]pyrido[3,2-d]pyrimidin-8-yl}-1H,5H,6H,7H-pyrrolo[3,2-c]pyridin-4-one (7.5 mg, 4.54%) as a yellow solid.

LC-MS: (M+H)⁺ found: 550.0.

¹H NMR (400 MHz, DMSO-d6) δ 11.95 (s, 1H), 9.50 (s, 1H), 8.71 (d, J=4.8 Hz, 1H), 7.89 (s, 1H), 7.59 (d, J=4.8 Hz, 1H), 7.26 (d, J=2.9 Hz, 1H), 6.71 (dd, J=8.1, 1.7 Hz, 1H), 6.66 (t, J=8.0 Hz, 1H), 6.16 (m, J=8.0, 1.7 Hz, 1H), 4.72 (t, J=5.7 Hz, 2H), 3.87 (s, 3H), 3.58 (t, J=4.6 Hz, 4H), 3.51-3.43 (m, 2H), 2.95 (t, J=6.8 Hz, 2H), 2.82 (t, J=5.7 Hz, 2H), 2.49-2.50 (m, 4H).

### Example 243. 3-[(3-chloro-2-methoxyphenyl)amino]-2-{2-[3-(morpholin-4-yl)propoxy]pyrido[3,2-d]pyrimidin-8-yl}-1H,5H,6H,7H-pyrrolo[3,2-c]pyridin-4-one (compound 505)

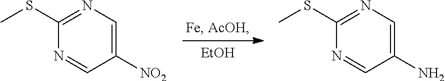

To a stirred solution of 2-(methylsulfanyl)-5-nitropyrimidine (5 g, 29.211 mmol, 1.00 equiv) in EtOH (200 mL) was added AcOH (120 mL) and Fe (17 g, 292.11 mmol, 10 equiv) in portions at rt under N₂ atmosphere. The resulting mixture was stirred for 2 h at 80 degrees C. under N₂ atmosphere. Desired product could be detected by LCMS. LC-MS: M+H found: 142. The resulting mixture was concentrated under reduced pressure. The resulting mixture was extracted with EA (3×100 mL). The combined organic layers were washed with Saturated NaCl (3×100 mL), dried over anhydrous Na₂SO₄. After filtration, the filtrate was concentrated under reduced pressure to afford 2-(methylsulfanyl)pyrimidin-5-amine (3.5 g, 84.86%) as a yellow solid.

LC-MS: M+H found: 142.0.

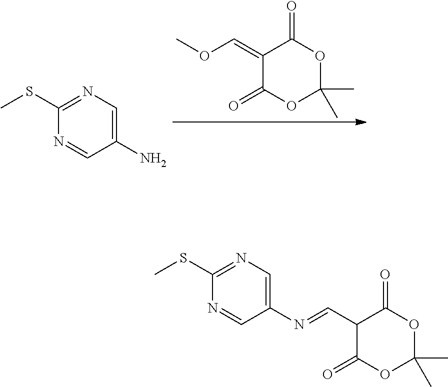

To a stirred solution of 2-(methylsulfanyl)pyrimidin-5-amine (3.2 g, 22.66 mmol, 1.00 equiv) and 5-(methoxymethylidene)-2,2-dimethyl-1,3-dioxane-4,6-dione (5.06 g, 27.18 mmol, 1.20 equiv) in DMF (80.00 mL) at rt under N2 atmosphere. The resulting mixture was stirred for 1 h at 80 degrees C. under N2atmosphere. Desired product could be detected by LCMS. LC-MS: M+H found: 296. The resulting mixture was added MeOH (50 mL). The resulting mixture was filtered, the filter cake was washed with MeOH (3×20 mL). The filter cake was concentrated under reduced pressure to afford 2,2-dimethyl-5-[(I E)-[[2-(methylsulfanyl)pyrimidin-5-yl]imino]methyl]-1,3-dioxane-4,6-dione (5.4 g, 80.68%) as a yellow solid.

LC-MS: (M+H)⁺ found: 296.0.

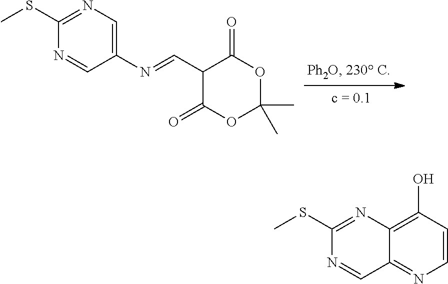

To a stirred solution of 2,2-dimethyl-5-[(1E)-{[2-(methylsulfanyl)pyrimidin-5-yl]imino}methyl]-1,3-dioxane-4,6-dione (5.3 g, 17.95 mmol, 1.00 equiv) in phenoxybenzene (360 mL) at rt under N2 atmosphere. The resulting mixture was stirred at 230 degrees C. under N2 atmosphere. Desired product could be detected by LCMS. LC-MS: M+H found: 194. The reaction was addition of Hexane (700 ml) at rt. The resulting mixture was filtered; the filter cake was washed with Hexane (3×200 ml). The filtrate was concentrated under reduced pressure.

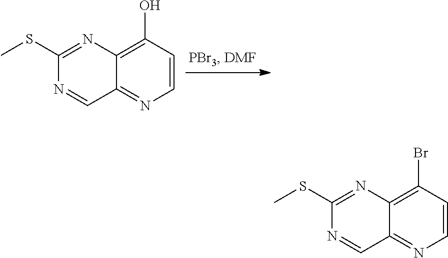

To a stirred solution of 2-(methylsulfanyl)pyrido[3,2-d]pyrimidin-8-ol (2.8 g, 14.49 mmol, 1.00 equiv) in DMF (80 mL) was added PBr3 (4.31 g, 15.94 mmol, 1.1 equiv) dropwise at 0 degrees C. under N2 atmosphere. The resulting mixture was stirred for 1 h at rt under N2 atmosphere. Desired product could be detected by LCMS. LC-MS: M+H found: 256. The resulting mixture was extracted with EA (3×100 mL). The combined organic layers were washed with aq. NaCl (3×100 mL), dried over anhydrous Na₂SO₄. After filtration, the filtrate was concentrated under reduced pressure. The residue was purified by silica gel column chromatography, eluted with PE:EA (4:1) to afford 8-bromo-2-(methylsulfanyl)pyrido[3,2-d]pyrimidine (1.6 g, 43.11%) as a white solid.

LC-MS: (M+H)⁺ found 256.0.

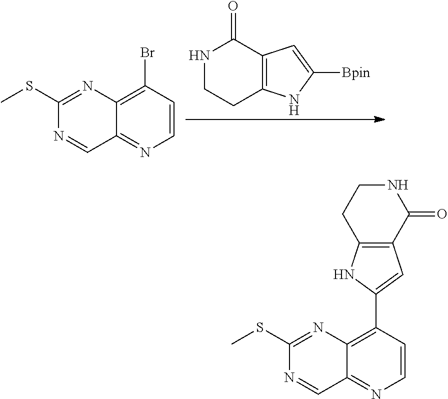

To a stirred solution of 8-bromo-2-(methylsulfanyl)pyrido[3,2-d]pyrimidine (700 mg, 2.73 mmol, 1.00 equiv) and 2-(4,4,5,5-tetramethyl-1,3,2-dioxaborolan-2-yl)-1H,5H,6H,7H-pyrrolo[3,2-c]pyridin-4-one (1075 mg, 4.10 mmol, 1.50 equiv) in dioxane (10 mL) and H₂O (1 mL) were added Na2CO3 (869.03 mg, 8.199 mmol, 3.00 equiv) and XPhos Pd G2 (215 mg, 0.27 mmol, 0.10 equiv) dropwise/in portions at rt under N₂ atmosphere. The resulting mixture was stirred for 2 h at 80 degrees C. under N₂ atmosphere. Desired product could be detected by LCMS. LC-MS: M+H found: 312. The resulting mixture was concentrated under vacuum. The residue was purified by silica gel column chromatography, eluted with DCM/MeOH=24:1 to afford 2-[2-(methylsulfanyl)pyrido[3,2-d]pyrimidin-8-yl]-1H,5H,6H,7H-pyrrolo[3,2-c]pyridin-4-one (700 mg, 82.26%) as a yellow solid.

LC-MS: (M+H)⁺ found: 312.0.

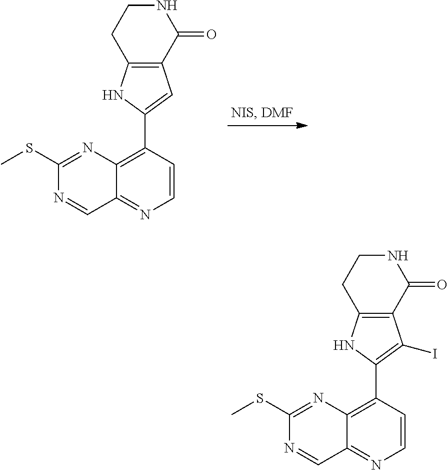 [SMILES: CSc1ncc2nccc(-c3cc4c([nH]3)CCNC4=O)c2n1 ; CSc1ncc2nccc(-c3[nH]c4c(c3I)C(=O)NCC4)c2n1]

To a stirred solution of 2-[2-(methylsulfanyl)pyrido[3,2-d]pyrimidin-8-yl]-1H,5H,6H,7H-pyrrolo[3,2-c]pyridin-4-one (600 mg, 1.93 mmol, 1.00 equiv) and NIS (650.33 mg, 2.891 mmol, 1.50 equiv) in DMF (10 mL) at rt under N2 atmosphere. The resulting mixture was stirred for overnight at 30 degrees C. under N2 atmosphere. Desired product could be detected by LCMS. LC-MS: M+H found: 438. The reaction was quenched by the addition of Saturated aq. Na2SO3 (20 mL) at 0 degrees C. The precipitated solids were collected by filtration and washed with H₂O (20 mL×3). The residue was purified by silica gel column chromatography, eluted with DCM:MeOH=10:1 to afford 3-iodo-2-[2-(methylsulfanyl)pyrido[3,2-d]pyrimidin-8-yl]-1H,5H,6H,7H-pyrrolo[3,2-c]pyridin-4-one (650 mg, 77.14%) as a yellow solid.

LC-MS: (M+H)⁺ found: 438.0.

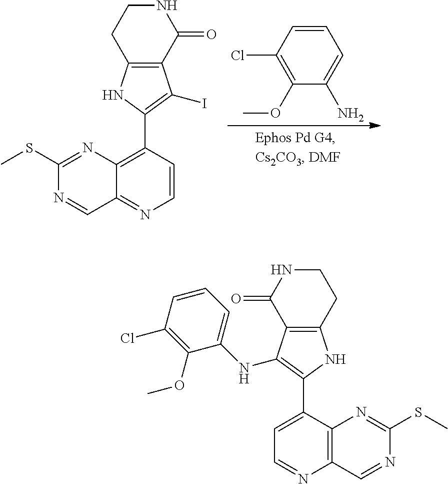

To a stirred solution of 3-iodo-2-[2-(methylsulfanyl)pyrido[3,2-d]pyrimidin-8-yl]-1H,5H,6H,7H-pyrrolo[3,2-c]pyridin-4-one (370 mg, 0.85 mmol, 1.00 equiv) and 3-chloro-2-methoxyaniline (134 mg, 0.85 mmol, 1 equiv) in DMF (4 mL) were added EPhos Pd G4 (78 mg, 0.08 mmol, 0.1 equiv) and Cs₂CO₃ (827 mg, 2.54 mmol, 3 equiv) in portions at rt under Ar atmosphere. The resulting mixture was stirred for 1.5 h at 50 degrees C. under Ar atmosphere. Desired product could be detected by LCMS. LC-MS: M+H found: 467. The resulting mixture was concentrated under vacuum. The residue was purified by silica gel column chromatography, eluted with DCM:MeOH (10:1) to afford 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (220 mg, 55.68%) as a orange solid.

LC-MS: (M+H)⁺ found: 467.0.

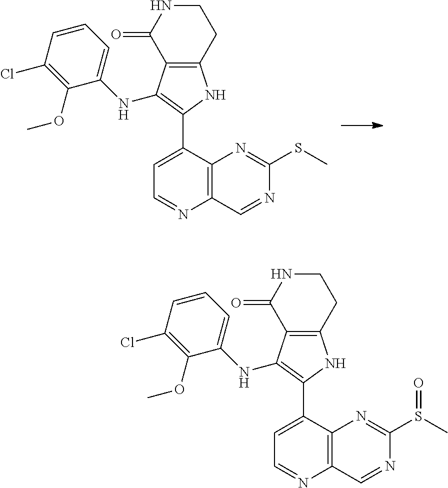 [SMILES: COc1c(Cl)cccc1Nc1c(-c2ccnc3cnc(SC)nc23)[nH]c2c1C(=O)NCC2 ; COc1c(Cl)cccc1Nc1c(-c2ccnc3cnc(S(C)=O)nc23)[nH]c2c1C(=O)NCC2]

To a stirred solution of 3-[(3-chloro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (50 mg, 0.11 mmol, 1.00 equiv) in DCM (2 mL, 31.46 mmol, 293.80 equiv) were added MCPBA (29 mg, 0.12 mmol, 1.1 equiv) dropwise/in portions at 0 degrees C. under N2 atmosphere. The resulting mixture was stirred for 1 h at 0 degrees C. under N2 atmosphere.

Desired product could be detected by LCMS. LC-MS: M+H found: 483. The resulting mixture was extracted with DCM (3×4 mL). The combined organic layers were dried over anhydrous Na₂SO₄. After filtration, the filtrate was concentrated under reduced pressure. The crude product was used in the next step directly without further purification.

LC-MS: (M+H)⁺ found: 483.0.

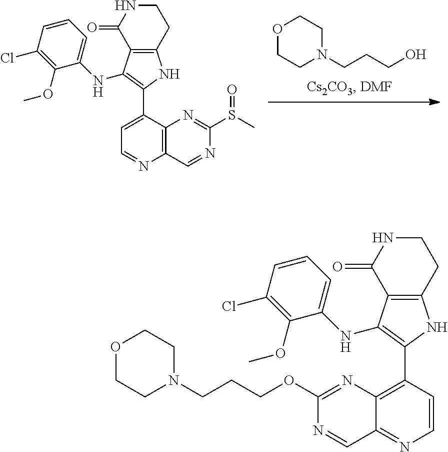 [SMILES: COc1c(Cl)cccc1Nc1c(-c2ccnc3cnc(S(C)=O)nc23)[nH]c2c1C(=O)NCC2 ; OCCCN1CCOCC1 ; COc1c(Cl)cccc1Nc1c(-c2ccnc3cnc(OCCCN4CCOCC4)nc23)[nH]c2c1C(=O)NCC2]

To a stirred solution of 3-[(3-chloro-2-methoxyphenyl)amino]-2-{2-methanesulfinylpyrido[3,2-d]pyrimidin-8-yl}-1H,5H,6H,7H-pyrrolo[3,2-c]pyridin-4-one (135 mg, 0.280 mmol, 1.00 equiv) and Cs₂CO₃ (218.59 mg, 0.672 mmol, 2.4 equiv) inDMF (2 mL) were added 3-(morpholin-4-yl)propan-1-ol (48.71 mg, 0.336 mmol, 1.2 equiv) dropwise at 0 degrees C. under N2 atmosphere. The resulting mixture was stirred for 1 h at RT under N2 atmosphere. Desired product could be detected by LCMS. LC-MS: M+H found: 564. The resulting mixture was concentrated under vacuum. The resulting mixture was concentrated under vacuum and dissolved in DMF. The crude product (100 mg) was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 22% B to 45% B in 10 min, 45% B; Wave Length: 254/220 nm; RT1 (min): 9.67) to 3-[(3-chloro-2-methoxyphenyl)amino]-2-{2-[3-(morpholin-4-yl)propoxy]pyrido[3,2-d]pyrimidin-8-yl}-1H,5H,6H,7H-pyrrolo[3,2-c]pyridin-4-one (10.3 mg, 6.53%) as a orange solid.

LC-MS: (M+H)⁺ found: 564.30.

¹H NMR (400 MHz, DMSO-d6) δ12.00 (s, 1H), 9.49 (s, 1H), 8.70 (d, J=4.8 Hz, 1H), 7.88 (s, 1H), 7.57 (d, J=4.8 Hz, 1H), 7.26 (d, J=2.7 Hz, 1H), 6.61-6.75 (m, 2H), 6.16 (m, J=7.9, 1.8 Hz, 1H)) 4.63 (t, J=6.6 Hz, 2H), 3.87 (s, 3H), 3.57 (t, J=4.6 Hz, 4H), 3.48 (s, 4H), 2.96 (t, J=6.8 Hz, 2H), 2.39 (s, 4H), 2.03 (m, J=6.9 Hz, 2H).

### Example 244. 3-[(3-chloro-2-methoxyphenyl) amino]-2-{6-[2-(morpholin-4-yl) ethoxy]-1,5-naphthyridin-4-yl}-1H,5H,6H,7H-pyrrolo[3,2-c] pyridin-4-one (compound 504)

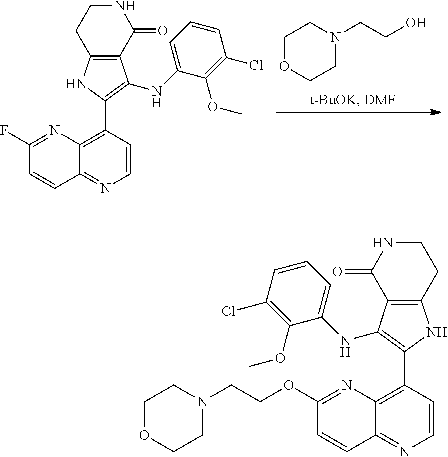

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (80 mg, 0.183 mmol, 1.00 equiv) in DMF (1.5 mL) was added 4-morpholineethanol (119.83 mg, 0.915 mmol, 5 equiv) in portions at degrees 0 C under nitrogen atmosphere. To the above mixture was added t-BuOK (22.55 mg, 0.201 mmol, 1.1 equiv) in portions over 30 min at room temperature. The crude product (80 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mIUmin; Gradient: 28% B to 58% B in 7 min; Wave Length: 254 nm; RT1 (min): 6.5) to afford 3-[(3-chloro-2-methoxyphenyl) amino]-2-{6-[2-(morpholin-4-yl) ethoxy]-1,5-naphthyridin-4-yl}-1H,5H,6H,7H-pyrrolo[3,2-c] pyridin-4-one as a yellow solid.

LC-MS: M+H found: 549.0.

¹H NMR (400 MHz, DMSO-d6) δ 12.02 (s, 1H), 8.59 (d, J=4.8 Hz, 1H), 8.29 (d, J=9.1 Hz, 1H), 7.74 (s, 1H), 7.52 (d, J=4.8 Hz, 1H), 7.35 (d, J=9.1 Hz, 1H), 7.20 (d, J=2.5 Hz, 1H), 6.72-6.53 (m, 2H), 6.19 (dd, J=7.7, 1.9 Hz, 1H), 4.70 (t, J=5.9 Hz, 2H), 3.84 (s, 3H), 3.57 (t, J=4.6 Hz, 4H), 3.47 (td, J=6.9, 2.5 Hz, 2H), 2.92 (t, J=6.8 Hz, 2H), 2.80 (t, J=5.9 Hz, 2H), 2.52 (s, 4H).

### Example 245. 3-[(3-chloro-2-methoxyphenyl) amino]-2-{6-[3-(morpholin-4-yl) propoxy]-1,5-naphthyridin-4-yl}-1H,5H,6H,7H-pyrrolo[3,2-c] pyridin-4-one (compound 503)

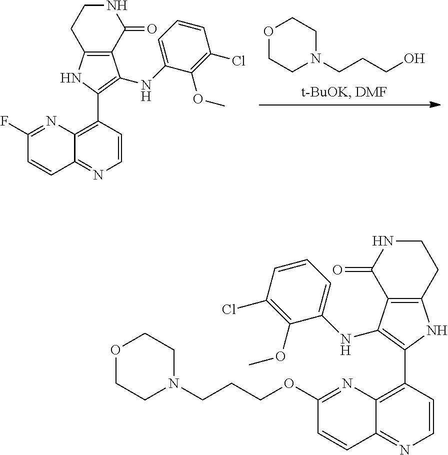

To a stirred solution of 3-[(3-chloro-2-methoxyphenyl) amino]-2-(6-fluoro-1,5-naphthyridin-4-yl)-1H,5H,6H,7H-pyrrolo[3,2-c] pyridin-4-one (80 mg, 0.183 mmol, 1.00 equiv) in DMF (1.5 mL, 19.383 mmol, 106.09 equiv) was added 3-(morpholin-4-yl) propan-1-ol (132.65 mg, 0.915 mmol, 5 equiv) in portions at degrees 0 C under nitrogen atmosphere. To the above mixture was added t-BuOK (22.55 mg, 0.201 mmol, 1.1 equiv) in portions over 30 min at room temperature. The crude product (80 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 32% B to 62% B in 7 min; Wave Length: 254 nm; RT1 (min): 6.5) to afford 3-[(3-chloro-2-methoxyphenyl) amino]-2-{6-[3-(morpholin-4-yl) propoxy]-1,5-naphthyridin-4-yl}-1H,5H,6H,7H-pyrrolo[3,2-c] pyridin-4-one (26.7 mg, 25.54%) as a yellow solid.

LC-MS: M+H found: 563.0.

¹H NMR (300 MHz, DMSO-d6) δ 12.14 (s, 1H), 8.59 (d, J=4.8 Hz, 1H), 8.31 (d, J=9.1 Hz, 1H), 7.77 (s, 1H), 7.52 (d, J=4.8 Hz, 1H), 7.40-7.14 (m, 2H), 6.79-6.58 (m, 2H), 6.19-6.17 (m, 1H), 4.61 (d, J=7.2 Hz, 2H), 3.85 (s, 3H), 3.74-3.38 (m, 6H), 2.96 (t, J=6.7 Hz, 2H), 2.53 (s, 4H), 2.39 (s, 2H), 2.05 (s, 2H).

### Example 246. 3-[(3-chloro-2-methoxyphenyl)amino]-2-(2-{[6-(4-methylpiperazin-1-yl)pyridin-3-yl]amino}pyrimidin-4-yl)-1H,5H,6H,7H-pyrrolo[3,2-c]pyridin-4-one (compound 518)

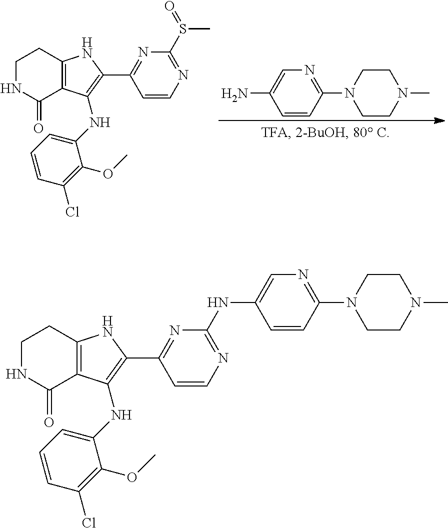 [SMILES: COc1c(Cl)cccc1Nc1c(-c2ccnc(S(C)=O)n2)[nH]c2c1C(=O)NCC2 ; CN1CCN(c2ccc(N)cn2)CC1 ; COc1c(Cl)cccc1Nc1c(-c2ccnc(Nc3ccc(N4CCN(C)CC4)nc3)n2)[nH]c2c1C(=O)NCC2]

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (80 mg, 0.185 mmol, 1.00 equiv) and 6-(4-methylpiperazin-1-yl)pyridin-3-amine (356.13 mg, 1.850 mmol, 10 equiv) in butan-2-ol (1.8 mL) was added TFA (42.24 mg, 0.370 mmol, 2 equiv) in portions at room temperature under nitrogen atmosphere. The resulting mixture was stirred for 2 days at 80 degrees C. under nitrogen atmosphere. Desired product could be detected by LCMS. The resulting mixture was concentrated under reduced pressure. The residue was purified by reverse flash chromatography with the following conditions: column, silica gel; mobile phase, MeCN in water, 10% to 50% gradient in 10 min; detector, UV 254 nm. This resulted in 3-[(3-chloro-2-methoxyphenyl)amino]-2-(2-{[6-(4-methylpiperazin-1-yl)pyridin-3-yl]amino}pyrimidin-4-yl)-1H,5H,6H,7H-pyrrolo[3,2-c]pyridin-4-one (17.3 mg, 15.86%) as a red solid.

LC-MS: (M+H)⁺ found: 560.3.

¹H NMR (400 MHz, DMSO-d6) δ 11.53 (s, 1H), 9.00 (s, 1H), 8.43 (d, J=2.7 Hz, 1H), 8.21 (d, J=5.3 Hz, 1H), 7.95 (dd, J=9.1, 2.8 Hz, 1H), 7.76 (s, 1H), 7.19 (d, J=2.8 Hz, 1H), 6.87-6.73 (m, 3H), 6.65 (d, J=5.4 Hz, 1H), 6.34 (dd, J=7.8, 1.9 Hz, 1H), 3.89 (s, 3H), 3.41 (td, J=6.9, 2.5 Hz, 4H), 2.89 (t, J=6.8 Hz, 2H), 2.76 (s, 3H), 2.55 (s, 6H).

### Example 247. 3-[(3-chloro-2-methoxyphenyl)amino]-2-(2-{[1-(oxan-4-yl)pyrazol-4-yl]amino}pyrimidin-4-yl)-1H,5H,6H,7H-pyrrolo[3,2-c]pyridin-4-one (compound 517)

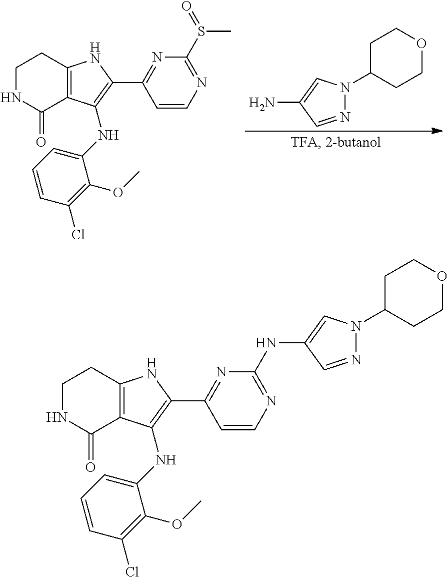 [SMILES: COc1c(Cl)cccc1Nc1c(-c2ccnc(S(C)=O)n2)[nH]c2c1C(=O)NCC2 ; Nc1cnn(C2CCOCC2)c1 ; COc1c(Cl)cccc1Nc1c(-c2ccnc(Nc3cnn(C4CCOCC4)c3)n2)[nH]c2c1C(=O)NCC2]

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (100 mg, 0.23 mmol, 1.00 equiv) and 1-(oxan-4-yl)pyrazol-4-amine (194 mg, 1.15 mmol, 5.00 equiv) in butan-2-ol (2.00 mL) was added TFA (53 mg, 0.46 mmol, 2.00 equiv) in portions at room temperature under nitrogen atmosphere. The resulting mixture was stirred for overnight at 80 degrees C. under nitrogen atmosphere. The reaction was monitored by LCMS. Desired product could be detected by LCMS. The resulting mixture was concentrated under reduced pressure. The crude product (100 mg) was purified by Prep-HPLC with the following conditions (Column: XBridge Prep OBD C18 Column, 30*150 mm, 5 μm; Mobile Phase A: Water (10 mmol/L NH4HCO₃), Mobile Phase B: ACN; Flow rate: 60 mL/min; Gradient: 25% B to 55% B in 7 min; Wave Length: 254 nm; RT1 (min): 6.5) to afford 3-[(3-chloro-2-methoxyphenyl)amino]-2-(2-([1-(oxan-4-yl)pyrazol-4-yl]amino)pyrimidin-4-yl)-1H,5H,6H,7H-pyrrolo[3,2-c]pyridin-4-one (78.8 mg, 61.39%) as a off-white solid.

LC-MS: (M+H)⁺ found: 534.95.

¹H NMR (300 MHz, DMSO-d6) h 11.50 (s, 1H), 9.08 (s, 1H), 8.20 (d, J=5.3 Hz, 1H), 7.99 (s, 1H), 7.73 (s, 1H), 7.61 (s, 1H), 7.21 (d, J=2.6 Hz, 1H), 6.86-6.75 (m, 1H), 6.75 (dd, J=8.0, 2.1 Hz, 1H), 6.59 (d, J=5.3 Hz, 1H), 6.32 (dd, J=7.5, 2.1 Hz, 1H), 4.33 (p, J=8.2 Hz, 1H), 3.97 (dt, J=11.7, 3.5 Hz, 2H), 3.91 (s, 3H), 3.52-3.37 (m, 4H), 2.90 (t, J=6.7 Hz, 2H), 1.96 (td, J=7.2, 5.3, 2.9 Hz, 4H).

### Example 248. 3-[(3-chloro-2-methoxyphenyl)amino]-2-{2-[(3-methyl-1,2,3-triazol-4-yl)amino]pyrimidin-4-yl}-1H,5H,6H,7H-pyrrolo[3,2-c]pyridin-4-one (compound 512)

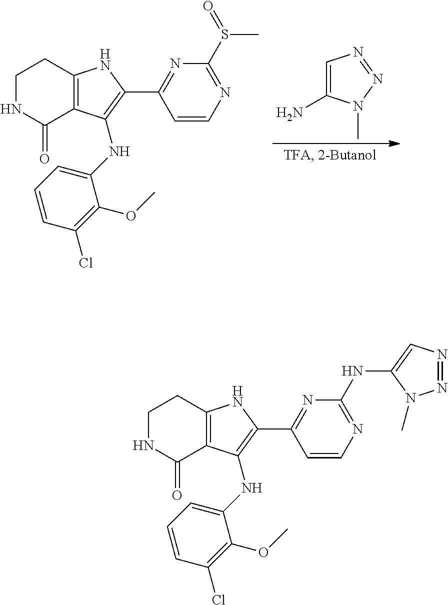

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (80 mg, 0.185 mmol, 1.00 equiv) and 3-methyl-1,2,3-triazol-4-amine (182 mg, 1.850 mmol, 10 equiv) in 2-Butanol (2 mL) was added TFA (42.24 mg, 0.370 mmol, 2 equiv) in portions at 80 degrees C. under nitrogen atmosphere. The resulting mixture was stirred for overnight at 80 degrees C. under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 22% B to 52% B in 7 min; Wave Length: 254 nm; RT1 (min): 6.5) to afford 3-[(3-chloro-2-methoxyphenyl)amino]-2-{2-[(3-methyl-1,2,3-triazol-4-yl)amino]pyrimidin-4-yl}-1H,5H,6H,7H-pyrrolo[3,2-c]pyridin-4-one (5.7 mg, 6.58%) as a light yellow solid.

LC-MS: M+H found: 466.0.

¹H NMR (300 MHz, DMSO-d6) δ 11.48 (s, 1H), 9.80 (s, 1H), 8.26 (dd, J=5.4, 2.7 Hz, 1H), 8.12 (d, J=2.6 Hz, 1H), 7.79 (d, J=2.5 Hz, 1H), 7.21 (d, J=2.8 Hz, 1H), 6.86-6.73 (m, 2H), 6.70 (dd, J=5.5, 2.6 Hz, 1H), 6.36-6.26 (m, 1H), 4.06 (d, J=2.6 Hz, 3H), 3.90 (d, J=2.8 Hz, 3H), 3.48-3.36 (m, 2H), 2.92 (dt, J=8.0, 4.0 Hz, 2H).

### Example 249. 3-[(3-chloro-2-methoxyphenyl)amino]-2-{2-[(1-methyl-1,2,3-triazol-4-yl)amino]pyrimidin-4-yl}-1H,5H,6H,7H-pyrrolo[3,2-c]pyridin-4-one (compound 511)

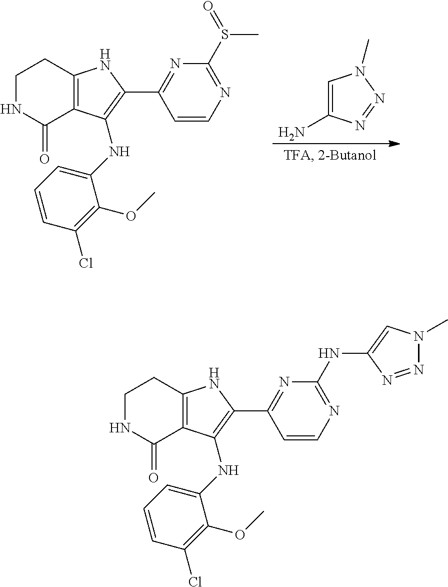

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (80 mg, 0.185 mmol, 1.00 equiv) and 1-methyl-1,2,3-triazol-4-amine (182 mg, 1.850 mmol, 10 equiv) in 2-Butanol (2 mL) was added TFA (42.24 mg, 0.370 mmol, 2 equiv) in portions at 80 degrees C. under nitrogen atmosphere. The resulting mixture was stirred for overnight at 80 degrees C. under nitrogen atmosphere. The resulting mixture was concentrated under reduced pressure. The crude product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 20% B to 44% B in 8 min, 44% B; Wave Length: 254/220 nm; RT1 (min): 7.55) to afford 3-[(3-chloro-2-methoxyphenyl)amino]-2-{2-[(1-methyl-1,2,3-triazol-4-yl)amino]pyrimidin-4-yl}-1H,5H,6H,7H-pyrrolo[3,2-c]pyridin-4-one (8.1 mg, 9.32%) as a light yellow solid.

LC-MS: M+H found: 466.0.

¹H NMR (400 MHz, DMSO-d6) δ 11.56 (s, 1H), 9.70 (s, 1H), 8.26 (d, J=5.4 Hz, 1H), 8.20 (s, 1H), 7.80 (s, 1H), 7.22 (d, J=2.6 Hz, 1H), 6.83-6.68 (m, 3H), 6.31 (dd, J=7.7, 2.0 Hz, 1H), 4.02 (s, 3H), 3.91 (s, 3H), 3.42 (td, J=6.8, 2.5 Hz, 2H), 2.91 (t, J=6.7 Hz, 2H).

### Example 250. 3-[(3-chloro-2-methoxyphenyl)amino]-2-{2-[(5-methyl-1,2-oxazol-4-yl)amino]pyrimidin-4-yl}-1H,5H,6H,7H-pyrrolo[3,2-c]pyridin-4-one (compound 510)

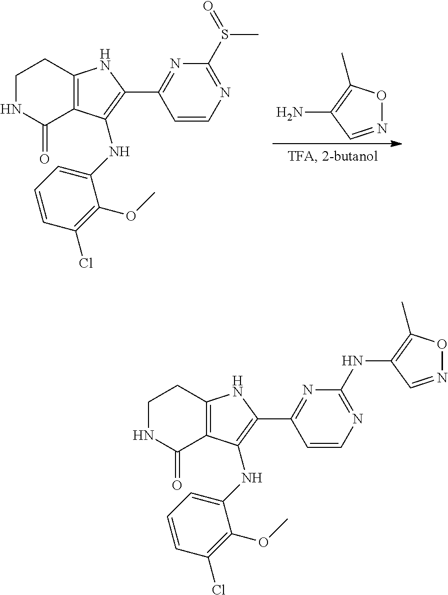

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (100 mg, 0.232 mmol, 1.00 equiv) in butan-2-ol (2.00 mL) were added 5-methyl-1,2-oxazol-4-amine (114 mg, 1.160 mmol, 5.00 equiv) and TFA (53 mg, 0.464 mmol, 2.00 equiv) in portions at room temperature under argon atmosphere. The resulting mixture was stirred for overnight at 80 degrees C. The reaction was monitored by LCMS. Desired product could be detected by LCMS. The resulting mixture was concentrated under reduced pressure. The crude product (100 mg) was purified by Prep-HPLC with the following conditions (Column. XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 28% B to 58% B in 7 min; Wave Length: 254 nm; RT1 (min): 6.5) to afford 3-[(3-chloro-2-methoxyphenyl)amino]-2-{2-[(5-methyl-1,2-oxazol-4-yl)amino]pyrimidin-4-yl}-1H,5H,6H,7H-pyrrolo[3,2-c]pyridin-4-one (72.2 mg, 64.66%) as a yellow solid.

LC-MS: (M+H)⁺ found: 465.90.

¹H NMR (300 MHz, DMSO-d6) δ 11.43 (s, 1H), 8.88 (s, 1H), 8.72 (s, 1H), 8.19 (d, J=5.3 Hz, 1H), 7.73 (s, 1H), 7.21 (d, J=2.5 Hz, 1H), 6.87-6.72 (m, 2H), 6.63 (d, J=5.3 Hz, 1H), 6.31 (dd, J=7.3, 2.3 Hz, 1H), 3.89 (s, 3H), 3.40 (dd, J=6.8, 2.5 Hz, 2H), 2.89 (t, J=6.8 Hz, 2H), 2.39 (s, 3H).

### Example 251. 3-[(3-chloro-2-methoxyphenyl)amino]-2-{2-[(1-methylpyrazol-3-yl)amino]pyrimidin-4-yl}-1H,5H,6H,7H-pyrrolo[3,2-c]pyridin-4-one (compound 509)

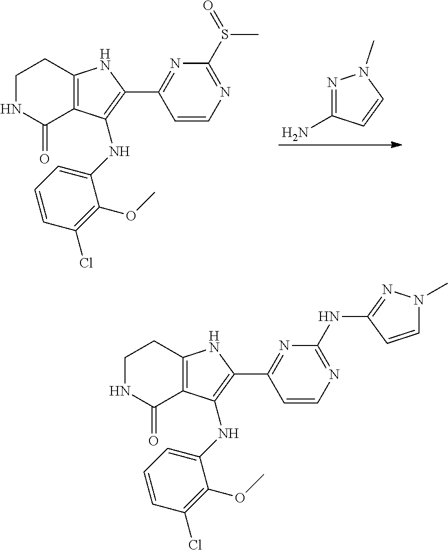

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (80 mg, 0.19 mmol, 1.00 equiv) and 1-methylpyrazol-3-amine (180 mg, 1.85 mmol, 10 equiv) in 2-Butanol (2 mL) was added TFA (42.24 mg, 0.370 mmol, 2 equiv) in portions at 80 degrees C. under nitrogen atmosphere. The resulting mixture was stirred for overnight at 80 degrees C. under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (0.1% FA), Mobile Phase B: ACN; Flow rate: 60 mL/min; Gradient: 12% B to 42% B in 7 min; Wave Length: 254 nm; RT1 (min): 6.5) to afford 3-[(3-chloro-2-methoxyphenyl)amino]-2-{2-[(1-methylpyrazol-3-yl)amino]pyrimidin-4-yl}-1H,5H,6H,7H-pyrrolo[3,2-c]pyridin-4-one (14.9 mg, 16.66%) as a yellow solid.

LC-MS: M+H found: 465.0.

¹H NMR (300 MHz, DMSO-d6) δ 11.53 (s, 1H), 9.21 (s, 1H), 8.22 (d, J=5.5 Hz, 1H), 7.94 (s, 1H), 7.55 (d, J=2.3 Hz, 1H), 7.18 (s, 1H), 6.88-6.75 (m, 2H), 6.68 (d, J=5.5 Hz, 1H), 6.59 (d, J=2.3 Hz, 1H), 6.39 (dd, J=7.0, 2.7 Hz, 1H), 3.88 (s, 3H), 3.75 (s, 3H), 3.41 (d, J=2.4 Hz, 2H), 2.89 (t, J=6.8 Hz, 2H).

### Example 252. 2-{3-amino-[1,2]oxazolo[4,5-b]pyridin-7-yl}-3-[(3-chloro-2-methoxyphenyl)amino]-1H,5H,6H,7H-pyrrolo[3,2-c]pyridin-4-one (compound 524)

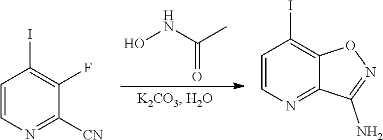

A mixture of 3-fluoro-4-iodopyridine-2-carbonitrile (2 g, 8.06 mmol, 1.00 equiv), acetohydroxamic acid (1.21 g, 16.13 mmol, 2.00 equiv) and K₂CO₃ (2.23 g, 16.13 mmol, 2 equiv) in H₂O (24 mL) was stirred overnight at room temperature under nitrogen atmosphere. Desired product could be detected by LCMS. The aqueous layer was extracted with CH₂Cl₂ (3×100 mL). The resulting mixture was concentrated under reduced pressure. The residue was purified by silica gel column chromatography, eluted with CH2Cl2/MeOH (12:1) to afford 7-iodo-[1,2]oxazolo[4,5-b]pyridin-3-amine (2 g, 95.01%) as a white solid.

LC-MS: [M−H]⁻ found: 261.85.

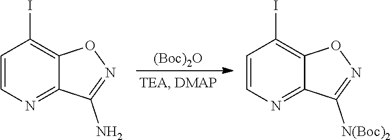

A mixture of 7-iodo-[1,2]oxazolo[4,5-b]pyridin-3-amine (1.2 g, 4.59 mmol, 1.00 equiv), di-tert-butyl dicarbonate (3.01 g, 13.79 mmol, 3.00 equiv), DMAP (0.28 g, 2.29 mmol, 0.50 equiv) and TEA (1.40 g, 13.79 mmol, 3.00 equiv) in DCM (12 mL) was stirred overnight at room temperature under nitrogen atmosphere. Desired product could be detected by LCMS. The resulting mixture was concentrated under reduced pressure. The residue was purified by silica gel column chromatography, eluted with CH₂Cl₂/MeOH (15:1) to afford tert-butyl N-(tert-butoxycarbonyl)-N-{7-iodo-[1,2]oxazolo[4,5-b]pyridin-3-yl}carbamate (1.65 g, 77.81%) as a white solid.

LC-MS: [M−H]⁻ found: 461.90.

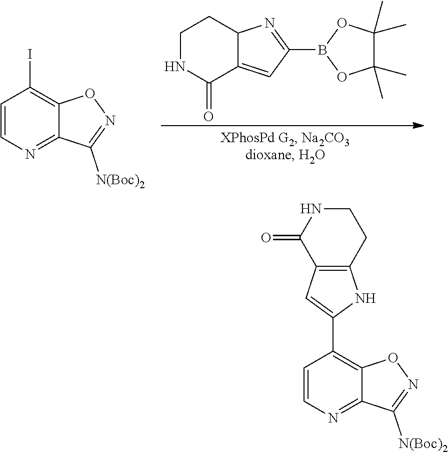

A mixture of tert-butyl N-(tert-butoxycarbonyl)-N-{7-iodo-[1,2]oxazolo[4,5-b]pyridin-3-yl}carbamate (0.8 g, 1.73 mmol, 1.00 equiv), 2-(4,4,5,5-tetramethyl-1,3,2-dioxaborolan-2-yl)-5H,6H,7H,7aH-pyrrolo[3,2-c]pyridin-4-one (0.68 g, 2.60 mmol, 1.50 equiv), 2nd Generation XPhos Precatalyst (0.14 g, 0.17 mmol, 0.10 equiv) and Na2CO3 (0.37 g, 3.47 mmol, 2.00 equiv) in 1,4-dioxane (12.00 mL) and water (3.00 mL) was stirred for 2 h at 50° C. under nitrogen atmosphere. Desired product could be detected by LCMS. The resulting mixture was concentrated under reduced pressure. The residue was purified by silica gel column chromatography, eluted with CH2Cl2/MeOH (20:1) to afford tert-butyl N-(tert-butoxycarbonyl)-N-(7-{4-oxo-1H,5H,6H,7H-pyrrolo[3,2-c]pyridin-2-yl}-[1,2]oxazolo[4,5-b]pyridin-3-yl)carbamate (0.7 g, 85.96%) as a yellow solid.

LC-MS: [M−H]⁻ found: 470.10.

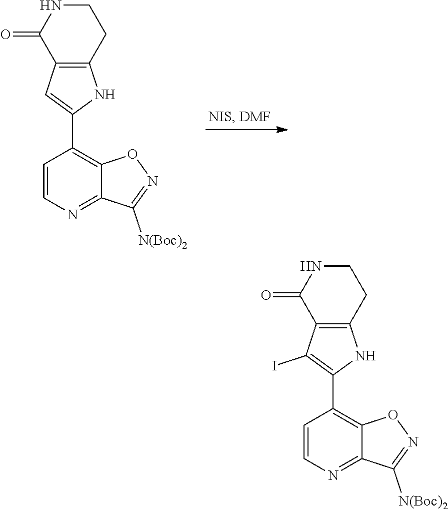

A mixture of tert-butyl N-(tert-butoxycarbonyl)-N-(7-{4-oxo-1H,5H,6H,7H-pyrrolo[3,2-c]pyridin-2-yl}-[1,2]oxazolo[4,5-b]pyridin-3-yl)carbamate (0.7 g, 1.491 mmol, 1 equiv) and NIS (1.01 g, 4.473 mmol, 3 equiv) in DMF (10 mL) was stirred for overnight at room temperature under nitrogen atmosphere. Desired product could be detected by LCMS. The resulting mixture was concentrated under reduced pressure. The residue was purified by silica gel column chromatography, eluted with CH₂Cl2/MeOH (20:1) to afford tert-butyl N-(tert-butoxycarbonyl)-N-(7-{3-iodo-4-oxo-1H,5H,6H,7H-pyrrolo[3,2-c]pyridin-2-yl}-[1,2]oxazolo[4,5-b]pyridin-3-yl)carbamate (210 mg, 23.66%) as a yellow solid.

LC-MS: [M+H]⁺ found: 596.10.

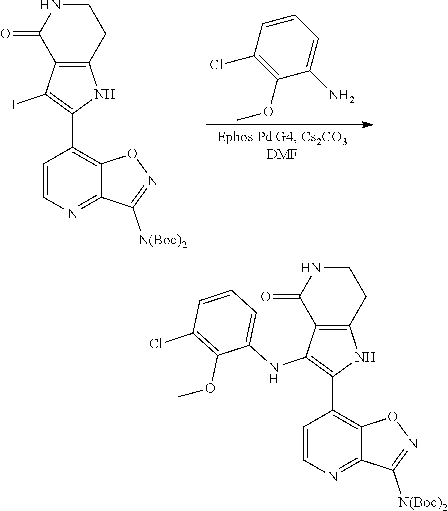

A mixture of tert-butyl N-(tert-butoxycarbonyl)-N-(7-{3-iodo-4-oxo-1H,5H,6H,7H-pyrrolo[3,2-c]pyridin-2-yl}-[1,2]oxazolo[4,5-b]pyridin-3-yl)carbamate (180 mg, 0.30 mmol, 1.00 equiv), 3-chloro-2-methoxyaniline (47 mg, 0.30 mmol, 1.00 equiv), EPhos Pd G4 (27 mg, 0.03 mmol, 0.10 equiv) and Cs₂CO₃ (197 mg, 0.60 mmol, 2.00 equiv) in DMF (2.00 mL) was stirred for 1 h at 50° C. under argon atmosphere. Desired product could be detected by LCMS. The resulting mixture was concentrated under reduced pressure. The residue was purified by silica gel column chromatography, eluted with CH2Cl2/MeOH (15:1) to afford tert-butyl N-(tert-butoxycarbonyl)-N-(7-{3-[(3-chloro-2-methoxyphenyl)amino]-4-oxo-1H,5H,6H,7H-pyrrolo[3,2-c]pyridin-2-yl}-[1,2]oxazolo[4,5-b]pyridin-3-yl)carbamate (120 mg, 63.50%) as a yellow solid.

LC-MS: [M+H]⁺ found: 625.10.

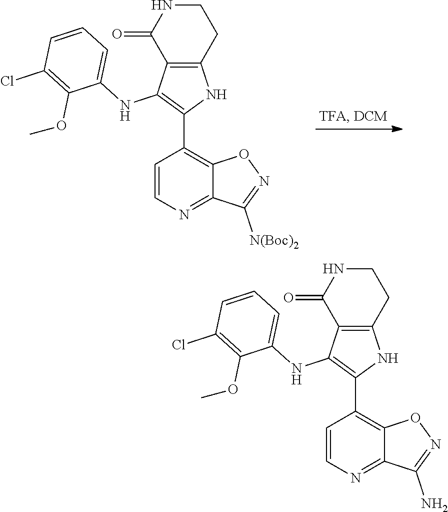

A mixture of tert-butyl N-(tert-butoxycarbonyl)-N-(7-{3-[(3-chloro-2-methoxyphenyl)amino]-4-oxo-1H,5H,6H,7H-pyrrolo[3,2-c]pyridin-2-yl}-[1,2]oxazolo[4,5-b]pyridin-3-yl)carbamate (120 mg, 0.19 mmol, 1.00 equiv) in TFA (2.00 mL) and DCM (2.00 mL) was stirred for 1 h at room temperature under air atmosphere.

Desired product could be detected by LCMS. The resulting mixture was concentrated under reduced pressure. The crude product was purified by Prep-HPLC with the following conditions (Column: XBridge Prep Phenyl OBD Column, 19*250 mm, 5 μm; Mobile Phase A: Water (10 mmol/L NH4HCO3+0.1% NH3·H2O), Mobile Phase B: ACN; Flow rate: 25 mL/min; Gradient: 33% B to 47% B in 10 min, 47% B; Wave Length: 254/220 nm; RT1 (min): 6.0) to afford 2-{3-amino-[1,2]oxazolo[4,5-b]pyridin-7-yl}-3-[(3-chloro-2-methoxyphenyl)amino]-1H,5H,6H,7H-pyrrolo[3,2-c]pyridin-4-one (16.4 mg, 19.79/0) as a yellow solid.

LC-MS: [M+H]⁺ found: 424.90.

¹H NMR (300 MHz, DMSO-d6) δ 11.70 (s, 1H), 8.37 (d, 1H), 7.61 (s, 1H), 7.32 (d, 1H), 7.23 (s, 1H), 6.77-6.63 (m, 2H), 6.56 (s, 2H), 6.19-6.13 (m, 1H), 3.92 (s, 3H), 3.50-3.39 (m, 2H), 3.08-2.89 (m, 2H).

### Example 253. 2-{3-amino-[1,2]oxazolo[4,5-b]pyridin-7-yl}-3-[(3-fluoro-2-methoxyphenyl)amino]-1H,5H,6H,7H-pyrrolo[3,2-c]pyridin-4-one; trifluoroacetic acid (compound 523)

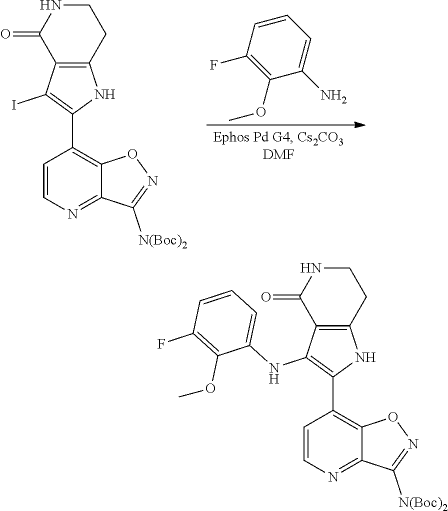

A mixture of tert-butyl N-(tert-butoxycarbonyl)-N-(7-{(3-iodo-4-oxo-1H,5H,6H,7H-pyrrolo[3,2-c]pyridin-2-yl}-[1,2]oxazolo[4,5-b]pyridin-3-yl)carbamate (150 mg, 0.25 mmol, 1.00 equiv), 3-fluoro-2-methoxyaniline (106 mg, 0.75 mmol, 3.00 equiv), EPhos Pd G4 (23 mg, 0.025 mmol, 0.10 equiv) and Cs₂CO₃ (164 mg, 0.50 mmol, 2.00 equiv) in DMF (2.00 mL) was stirred for 2 h at 50° C. under argon atmosphere. The resulting mixture was concentrated under reduced pressure. The residue was purified by silica gel column chromatography, eluted with CH₂Cl₂/MeOH (20:1) to afford tert-butyl N-(tert-butoxycarbonyl)-N-(7-{3-[(3-fluoro-2-methoxyphenyl)amino]-4-oxo-1H,5H,6H,7H-pyrrolo[3,2-c]pyridin-2-yl}-[1,2]oxazolo[4,5-b]pyridin-3-yl)carbamate (110 mg, 71.74%) as a yellow solid.

LC-MS: [M+H]⁺ found: 609.10.

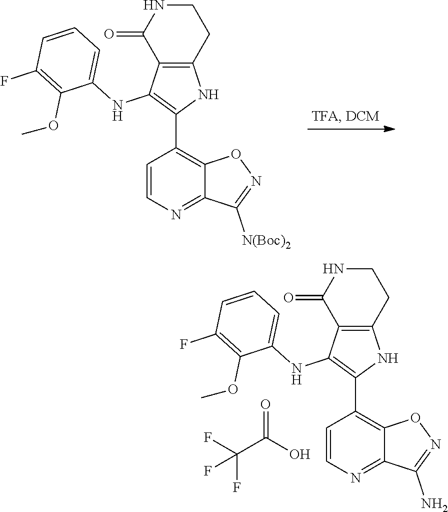

A solution of tert-butyl N-(tert-butoxycarbonyl)-N-(7-{3-[(3-fluoro-2-methoxyphenyl)amino]-4-oxo-1H,5H,6H,7H-pyrrolo[3,2-c]pyridin-2-yl}-[1,2]oxazolo[4,5-b]pyridin-3-yl)carbamate (110 mg, 0.18 mmol, 1.00 equiv) in DCM (2.00 mL) was added TFA (2.00 mL) at 0° C. The reaction was stirred for 1 h at room temperature under argon atmosphere. The resulting mixture was concentrated under reduced pressure. The crude product was purified by Prep-HPLC with the following conditions (Column: Xselect CSH C18 OBD Column 30*150 mm 5 μm, n; Mobile Phase A: Water (0.05% TFA), Mobile Phase B: ACN; Flow rate: 60 mL/min; Gradient: 17% B to 47% B in 7 min, 47% B; Wave Length: 254 nm; RT1 (min): 6.27) to afford 2-{3-amino-[1,2]oxazolo[4,5-b]pyridin-7-yl}-3-[(3-fluoro-2-methoxyphenyl)amino]-1H,5H,6H,7H-pyrrolo[3,2-c]pyridin-4-one; trifluoroacetic acid (27.8 mg, 29.38%) as an orange solid.

LC-MS: [M+H]⁺ found: 409.00.

¹H NMR (300 MHz, DMSO-d₆) δ 11.62 (s, 1H), 8.37 (d, 1H), 7.60 (s, 1H), 7.31 (d, 1H), 7.24 (s, 1H), 6.67-6.48 (m, 3H), 6.00 (d, 1H), 3.95 (s, 3H), 3.42 (t, 2H), 2.93 (t, 2H). 19F NMR (300 MHz, DMSO-d6) δ −74.76, −132.60.

### Example 254. 3-[(3-chloro-2-methoxyphenyl)amino]-2-{2-[(1-isopropylpyrazol-4-yl)amino]pyrimidin-4-yl}-1H,5H,6H,7H-pyrrolo[3,2-c]pyridin-4-one (compound 516)

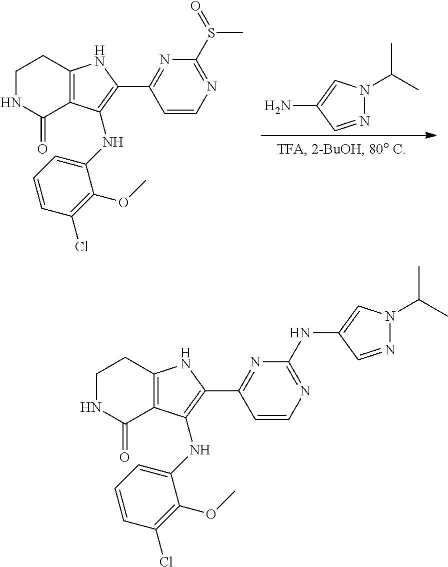

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (70 mg, 0.162 mmol, 1.00 equiv) and 1-isopropylpyrazol-4-amine (101.44 mg, 0.810 mmol, 5 equiv) in butan-2-ol (1.8 mL, 24.284 mmol) was added TFA (36.96 mg, 0.324 mmol, 2 equiv) in portions at room temperature under nitrogen atmosphere. The resulting mixture was stirred for overnight at 80 degrees C. under nitrogen atmosphere. The resulting mixture was stirred for overnight at 80 degrees C. under nitrogen atmosphere. Desired product could be detected by LCMS. The resulting mixture was concentrated under reduced pressure. The crude product (40 mg) was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 19% B to 43% B in 8 min, 43% B; Wave Length: 254/220 nm; RT1 (min): 8) to afford 3-[(3-chloro-2-methoxyphenyl)amino]-2-{2-[(1-isopropylpyrazol-4-yl)amino]pyrimidin-4-yl}-1H,5H,6H,7H-pyrrolo[3,2-c]pyridin-4-one (10.5 mg, 13.04%) as a yellow solid.

LC-MS: (M+H)⁺ found: 492.95.

¹H NMR (300 MHz, DMSO-d6) δ 11.50 (s, 1H), 9.06 (s, 1H), 8.19 (d, J=5.3 Hz, 1H), 7.97 (s, 1H), 7.72 (s, 1H), 7.59 (s, 1H), 7.20 (s, 1H), 6.89-6.69 (m, 2H), 6.57 (d, J=5.3 Hz, 1H), 6.32 (dd, J=7.5, 2.1 Hz, 1H), 4.45 (p, J=6.6 Hz, 1H), 3.90 (s, 3H), 3.40 (t, J=6.8 Hz, 2H), 2.90 (t, J=6.8 Hz, 2H), 1.41 (d, J=6.6 Hz, 6H).

### Example 255. 3-[(3-chloro-2-methoxyphenyl)amino]-2-(2-{[1-(2,2-difluoroethyl)pyrazol-3-yl]amino}pyrimidin-4-yl)-1H,5H,6H,7H-pyrrolo[3,2-c]pyridin-4-one (compound 515)

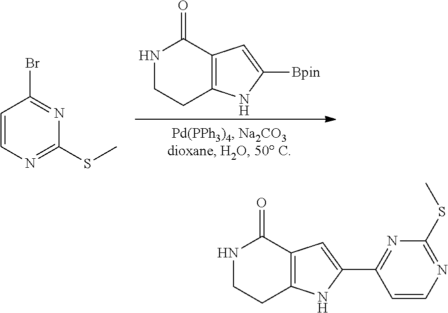

To a solution of 4-bromo-2-(methylsulfanyl)pyrimidine (4.00 g, 19.5 mmol, 1.00 equiv) and 2-(4,4,5,5-tetramethyl-1,3,2-dioxaborolan-2-yl)-1H,5H,6H,7H-pyrrolo[3,2-c]pyridin-4-one (6.14 g, 23.4 mmol, 1.20 equiv) in H₂O (40 mL) and dioxane (200 mL) were added Na2CO3 (4.13 g, 39.0 mmol, 2.00 equiv) and Pd(PPh₃)₄ (2.25 g, 1.95 mmol, 0.10 equiv). After stirring for 2 h at 50 degrees C. under a nitrogen atmosphere, the resulting mixture was concentrated under reduced pressure. The residue was purified silica gel column chromatography, eluted with CH₂Cl₂/MeOH (20:1) to afford 2-[2-(methylsulfanyl)pyrimidin-4-yl]-1H,5H,6H,7H-pyrrolo[3,2-c]pyridin-4-one (4 g, 78.78%) as a white solid.

LC-MS: (M+H)⁺ found: 261.00.

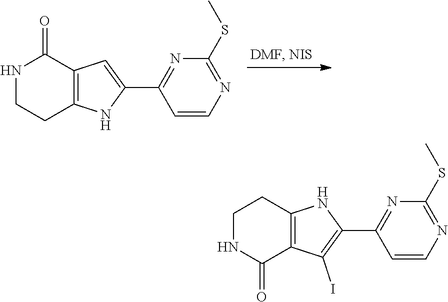 [SMILES: CSc1nccc(-c2cc3c([nH]2)CCNC3=O)n1 ; CSc1nccc(-c2[nH]c3c(c2I)C(=O)NCC3)n1]

Into a 40-mL vial, was placed 2-[2-(methylsulfanyl) pyrimidin-4-yl]-1H,5H,6H,7H-pyrrolo[3,2-c] pyridin-4-one (1.00 g, 3.84 mmol, 1.00 equiv), DMF (10 mL), NIS (1.04 g, 4.61 mmol, 1.20 equiv). The resulting solution was stirred for 5 h at room temperature. The reaction was monitored by LCMS. Desired product could be detected by LCMS. The reaction was quenched with sat. Na2SO3 (aq.) at 0 degrees C. The precipitated solids were collected by filtration and washed with water (3×50 mL). This resulted in 1.3 g (85.87%) of 3-iodo-2-[2-(methylsulfanyl) pyrimidin-4-yl]-1H,5H,6H,7H-pyrrolo[3,2-c] pyridin-4-one as a white solid. LC-MS: (M+H)⁺ found 386.80.

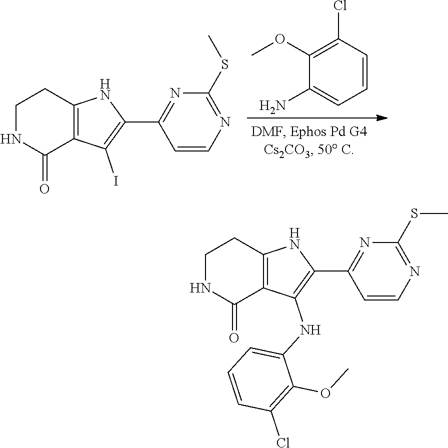

To a stirred mixture of 3-iodo-2-[2-(methylsulfanyl)pyrimidin-4-yl]-1H,5H,6H,7H-pyrrolo[3,2-c]pyridin-4-one (1.00 g, 2.59 mmol, 1.00 equiv) and 3-chloro-2-methoxyaniline (408 mg, 2.59 mmol, 1.00 equiv) in dioxane (26 mL) were added Ephos Pd G4 (2378 mg, 0.26 mmol, 0.10 equiv) and Cs₂CO₃ (1.69 g, 5.18 mmol, 2.00 equiv) in portions at room temperature under argon atmosphere. The resulting mixture was stirred for overnight at 50 degrees C. under argon atmosphere. The reaction was monitored by LCMS. Desired product could be detected by LCMS. The resulting mixture was filtered, the filter cake was washed with DCM (2×50 mL). The filtrate was concentrated under reduced pressure. The resulting mixture was diluted with DMF (5 mL). The residue was purified by reverse flash chromatography with the following conditions: column, C18 silica gel; mobile phase, NH3·H2O in water, 10% to 50% gradient in 10 min: detector, UV 254 nm. This resulted in 580 mg (43.63%) of 3-[(3-chloro-2-methoxyphenyl)amino]-2-[2-(methylsulfanyl)pyrimidin-4-yl]-1H,5H,6H,7H-pyrolo[3,2-c]pyridin-4-one as a yellow solid.

LC-MS: (M+H)⁺ found: 416.10.

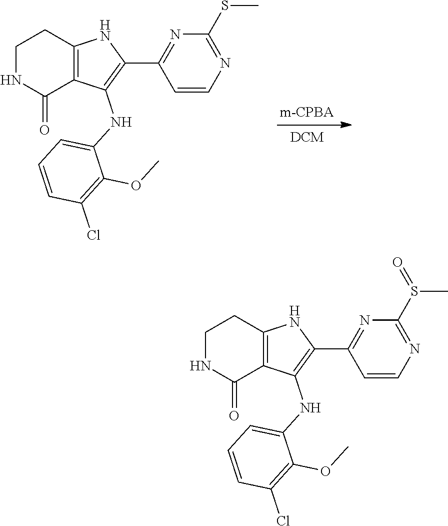 [SMILES: COc1c(Cl)cccc1Nc1c(-c2ccnc(SC)n2)[nH]c2c1C(=O)NCC2 ; COc1c(Cl)cccc1Nc1c(-c2ccnc(S(C)=O)n2)[nH]c2c1C(=O)NCC2]

To a stirred mixture of 3-[(3-chloro-2-methoxyphenyl)amino]-2-[2-(methylsulfanyl)pyrimidin-4-yl]-1H,5H,6H,7H-pyrrolo[3,2-c]pyridin-4-one (500 mg, 1.20 mmol, 1.00 equiv) in DCM (6 mL) was added m-CPBA (311 mg, 1.80 mmol, 1.50 equiv) in DCM (6 mL) dropwise at 0° C. under air atmosphere. The resulting mixture was stirred for 1 h at 0° C. under air atmosphere. The reaction was quenched by the addition of sat. Na2SO3 (aq.) (5 mL) at 0° C. The precipitated solids were collected by filtration and washed with water (1×5 mL). The resulting mixture was washed with 1×5 mL of MeOH. The resulting mixture was washed with 1×5 mL of ethyl ether to afford 3-[(3-chloro-2-methoxyphenyl)amino]-2-(2-methanesulfinylpyrimidin-4-yl)-1H,5H,6H,7H-pyrrolo[3,2-c]pyridin-4-one (400 mg, 77.04%) as a light yellow solid.

LC-MS: (M+H)⁺ found: 432.10.

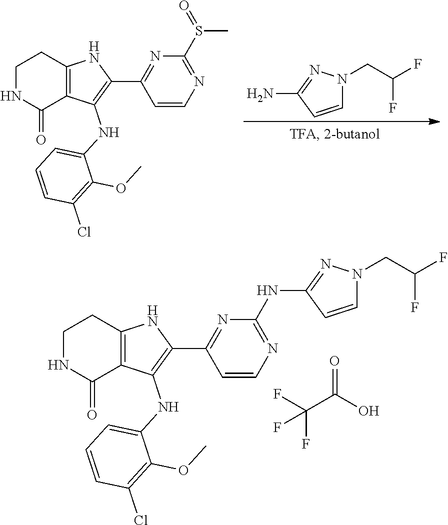

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (350 mg, 0.81 mmol, 1.00 equiv) and 1-(2,2-difluoroethyl)pyrazol-3-amine (596 mg, 4.05 mmol, 5.00 equiv) in butan-2-ol (5.00 mL) was added TFA (185 mg, 1.62 mmol, 2.00 equiv) in portions at room temperature under nitrogen atmosphere. The resulting mixture was stirred for overnight at 80 degrees C. under nitrogen atmosphere. The reaction was monitored by LCMS. Desired product could be detected by LCMS. The resulting mixture was concentrated under reduced pressure. The crude product (350 mg) was purified by Prep-HPLC with the following conditions (Column: XSelect CSH Prep C18 OBD Column, 19*250 mm, 5 m; Mobile Phase A: Water (0.05% FA), Mobile Phase B: MeOH-HPLC; Flow rate: 25 mL/min; Gradient: 34% B to 39% B in 8 min, 39% B; Wave Length: 254 nm; RT1 (min): 6.88) to afford 3-[(3-chloro-2-methoxyphenyl)amino]-2-(2-([1-(2,2-difluoroethyl)pyrazol-3-yl]amino)pyrimidin-4-yl)-1H,5H,6H,7H-pyrrolo[3,2-c]pyridin-4-one (80.7 mg, 18.45%) as a yellow solid.

LC-MS: (M+H)⁺ found: 515.30.

¹H NMR (400 MHz, DMSO-d6) δ 11.76 (s, 1H), 10.23 (s, 1H), 8.21 (d, J=6.0 Hz, 2H), 7.73 (d, J=2.4 Hz, 1H), 7.22 (s, 1H), 6.93-6.75 (m, 3H), 6.66-6.19 (m, 3H), 4.62-4.50 (m, 2H), 3.85 (s, 3H), 3.50-3.32 (m, 2H), 2.90 (t, J=6.7 Hz, 2H).

### Example 256. 3-[(3-chloro-2-methoxyphenyl)amino]-2-(2-{[1-(2,2-difluoroethyl)pyrazol-4-yl]amino}pyrimidin-4-yl)-1H,5H,6H,7H-pyrrolo[3,2-c]pyridin-4-one (compound 514)

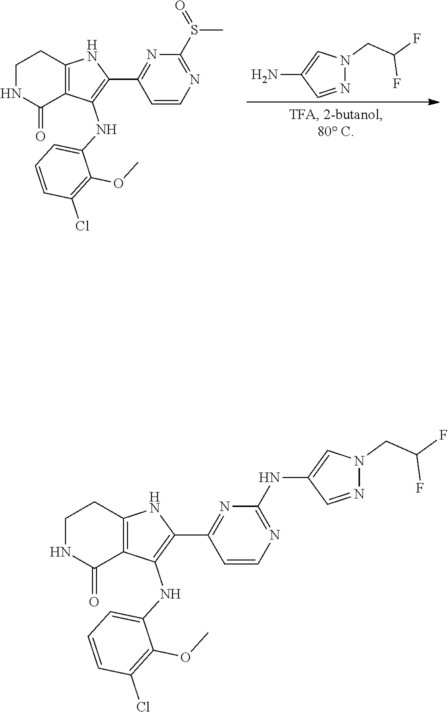 [SMILES: COc1c(Cl)cccc1Nc1c(-c2ccnc(S(C)=O)n2)[nH]c2c1C(=O)NCC2 ; Nc1cnn(CC(F)F)c1 ; COc1c(Cl)cccc1Nc1c(-c2ccnc(Nc3cnn(CC(F)F)c3)n2)[nH]c2c1C(=O)NCC2]

A solution of 3-[(3-chloro-2-methoxyphenyl)amino]-2-(2-methanesulfinylpyrimidin-4-yl)-1H,5H,6H,7H-pyrrolo[3,2-c]pyridin-4-one (70 mg, 0.16 mmol, 1.00 equiv) in butan-2-ol (3.50 mL) was added 1-(2,2-difluoroethyl) pyrazol-4-amine (71 mg, 0.48 mmol, 3.00 equiv) and TFA (37 mg, 0.32 mmol, 2.00 equiv) dropwise at room temperature under N₂ atmosphere. The resulting mixture was stirred for 2 h at 80 degrees C. under N2 atmosphere. The reaction was monitored by LCMS. Desired product could be detected by LCMS. The crude product was purified by Prep-HPLC with the following conditions (Column: XBridge Prep OBD C18 Column, 30*150 mm, 5pim; Mobile Phase A: Water (10 mmol/L NH4HCO3), Mobile Phase B: ACN; Flow rate: 60 mL/min; Gradient: 29% B to 59% B in 7 min; Wave Length: 254 nm; RT1 (min): 6.5; Number Of Runs: 2) to afford 3-[(3-chloro-2-methoxyphenyl)amino]-2-(2-([1-(2,2-difluoroethyl)pyrazol-4-yl]amino)pyrimidin-4-yl)-1H,5H,6H,7H-pyrrolo[3,2-c]pyridin-4-one (22 mg, 26.44%) as an off-white solid.

LC-MS: (M+H)⁺ found: 514.95.

¹H NMR (300 MHz, DMSO-d₆) δ 11.44 (s, 1H), 9.17 (s, 1H), 8.21 (d, J=5.1 Hz, 1H), 8.08 (s, 1H), 7.69 (d, J=7.8 Hz, 2H), 7.21 (s, 1H), 6.83-6.74 (m, 2H), 6.60-6.54 (m, 1H), 6.37-6.30 (m, 2H), 4.62-4.51 (m, 2H), 3.91 (s, 3H), 3.43-3.40 (m, 2H), 2.90 (t, J=6.6 Hz, 2H).

### Example 257. 3-[(3-chloro-2-methoxyphenyl)amino]-2-{2-[(1,5-dimethylpyrazl-3-yl)amino]pyrimidin-4-yl}-1H,5H,6H,7H-pyrrolo[3,2-c]pyridin-4-one (compound 513)

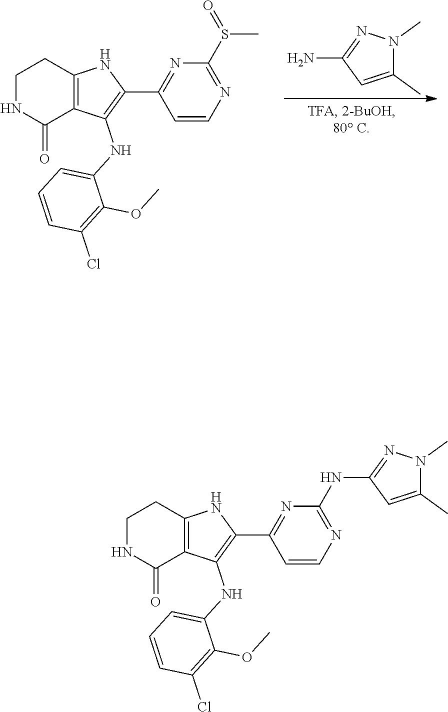

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (80 mg, 0.185 mmol, 1.00 equiv) and 1,5-dimethylpyrazol-3-amine (41.18 mg, 0.370 mmol, 2 equiv) in butan-2-ol (1.8 mL) was added TFA (42.24 mg, 0.370 mmol, 2 equiv) in portions at room temperature under nitrogen atmosphere. The resulting mixture was stirred for overnight at 80 degrees C. under nitrogen atmosphere. Desired product could be detected by LCMS. The resulting mixture was concentrated under reduced pressure. The crude product (80 mg) was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 17% B to 27% B in 9 min, 27% B; Wave Length: 254/220 nm; RT1 (min): 8.13) to afford 3-[(3-chloro-2-methoxyphenyl)amino]-2-{2-[(1,5-dimethylpyrazol-3-yl)amino]pyrimidin-4-yl}-1H,5H,6H,7H-pyrrolo[3,2-c]pyridin-4-one (30.2 mg, 33.06%) as a yellow solid.

LC-MS: (M+H)⁺ found: 478.95.

¹H NMR (300 MHz, DMSO-d6) δ 11.50 (s, 1H), 8.37 (s, 1H), 8.10 (d, J=5.5 Hz, 1H), 7.90 (s, 1H), 7.52 (s, 1H), 7.17 (s, 1H), 6.95-6.73 (m, 2H), 6.60 (d, J=5.5 Hz, 1H), 6.47-6.28 (m, 1H), 3.86 (s, 3H), 3.71 (s, 3H), 3.42 (s, 2H), 2.88 (t, J=6.7 Hz, 2H), 2.14 (s, 3H).

### Example 258. 3-[(3-chloro-2-methoxyphenyl)amino]-2-(2-{[3-(methanesulfonylmethyl)-1,2,3-triazol-4-yl]amino}pyrimidin-4-yl)-1H,5H,6H,7H-pyrrolo[3,2-c]pyridin-4-one (compound 539)

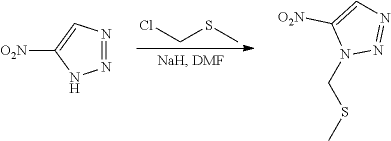

To a stirred solution of 4-nitro-3H-1,2,3-triazole (2.00 g, 17.53 mmol, 1 equiv) in DMF (20 mL) were added NaH (0.91 g, 22.79 mmol, 1.30 equiv, 60%) at 0 degrees C. under N2 atmosphere. The resulting mixture was stirred for 0.5 h at 0 degrees C. To the above mixture was added chloromethyl methyl sulfide (2.71 g, 28.05 mmol, 1.60 equiv) in portions over 0° C. at N₂. The resulting mixture was stirred for additional 4 h at room temperature. The reaction was monitored by TCL and LCMS. The resulting solution was decolorized by the addition of H2O. The resulting mixture was extracted with EA (3×60 mL). The combined organic layers were washed with NaCl (3×40 mL), dried over anhydrous Na₂SO₄. After filtration, the filtrate was concentrated under reduced pressure. The residue was purified by silica gel column chromatography, eluted with PE/EA (9/1) to afford 1-[(methylsulfanyl)methyl]-5-nitro-1,2,3-triazole (900 mg, 29.17%) as a white oil.

LC-MS: M+H found: 175.00.

¹H NMR (400 MHz, Chloroform-d) δ 8.15 (s, 1H), 5.41 (s, 2H), 2.25 (s, 3H).

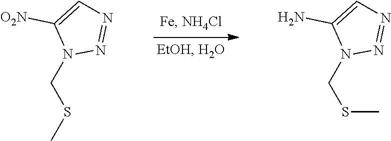

To a solution of 1-[(methylsulfanyl)methyl]-5-nitro-1,2,3-triazole (0.20 g, 1.15 mmol, 1.00 equiv) and Fe (320 mg, 5.74 mmol, 5.00 equiv) in EtOH (4 mL) and H₂O (1 mL) were added NH₄Cl (614 mg, 11.48 mmol, 10 equiv) and stirred at 70° C. for 2 h. The resulting mixture was filtered, the filtrate was concentrated under vacuum. The residue was purified by silica gel column chromatography, eluted with PE/EA (1/1) to afford 3-[(methylsulfanyl)methyl]-1,2,3-triazol-4-amine (140 mg, 83.71%) as a yellow solid.

LC-MS: M+H found: 145.05.

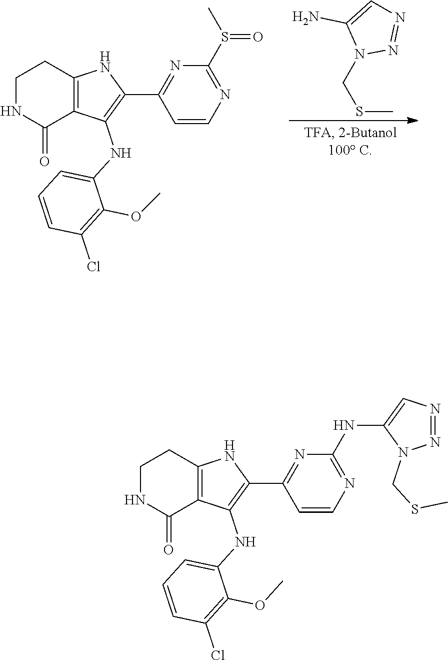

To a stirred solution of 3-[(methylsulfanyl)methyl]-1,2,3-triazol-4-amine (100 mg, 0.69 mmol, 1.00 equiv) and 3-[(3-chloro-2-methoxyphenyl)amino]-2-(2-methanesulfinylpyrimidin-4-yl)-1H,5H,6H,7H-pyrrolo[3,2-c]pyridin-4-one (75 mg, 0.17 mmol, 0.25 equiv) in 2-butyl-alcohol (3 mL) were added 2-butyl-alcohol (3 mL) and TFA (52.9 mg, 0.46 mmol, 0.67 equiv) at 100 degrees C. under N2 atmosphere. The resulting mixture was stirred for overnight at 100 degrees C. The reaction was monitored by TLC and LCMS. The resulting mixture was concentrated under vacuum. The mixture was neutralized to pH 7 with Na2CO3. The resulting mixture was extracted with DCM/MeOH=10/1 (3×50 mL). The combined organic layers were washed with brine (3×50 mL), dried over anhydrous Na₂SO₄. The resulting mixture was filtered and concentrated under reduced pressure. The residue was washed with DCM (2 mL), the precipitated solids were collected by filtration and washed with DCM (3×2 mL) to afford 3-[(3-chloro-2-methoxyphenyl)amino]-2-[2-({3-[(methylsulfanyl)methyl]-1,2,3-triazol-4-yl}amino)pyrimidin-4-yl]-1H,5H,6H,7H-pyrrolo[3,2-c]pyridin-4-one (100 mg, 9.01%) as a yellow solid.

LC-MS: M+H found: 512.05.

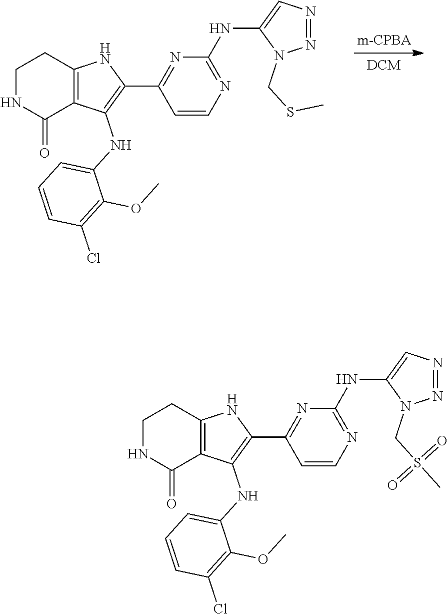 [SMILES: COc1c(Cl)cccc1Nc1c(-c2ccnc(Nc3cnnn3CSC)n2)[nH]c2c1C(=O)NCC2 ; COc1c(Cl)cccc1Nc1c(-c2ccnc(Nc3cnnn3CS(C)(=O)=O)n2)[nH]c2c1C(=O)NCC2]

To a stirred solution of 3-[(3-chloro-2-methoxyphenyl)amino]-2-[2-({3-[(methylsulfanyl)methyl]-1,2,3-triazol-4-yl}amino)pyrimidin-4-yl]-1H,5H,6H,7H-pyrrolo[3,2-c]pyridin-4-one (70 mg, 0.14 mmol, 1.00 equiv) in DCM (3 mL) were added m-CPBA (58.9 mg, 0.34 mmol, 2.50 equiv) at 0 degrees C. under N2 atmosphere. The resulting mixture was stirred for 2 h at 25 degrees C. The reaction was monitored by TCL and LCMS. The resulting solution was decolorized by the addition of Na2SO3. The resulting mixture was extracted with DCM/MeOH=10/1 (3×50 mL). The combined organic layers were washed with NaCl (3×50 mL), dried over anhydrous Na₂SO₄. The resulting mixture was concentrated under vacuum. The crude product (150 mg) was purified by Prep-HPLC with the following conditions ((2 #SHIMADZU (HPLC-01)): Column, XSelect CSH Prep C18 OBD Column, 19*250 mm, 5 μm; mobile phase, Water (0.1% FA) and ACN (35% ACN up to 65% in 7 min); Detector, UV (254 nm) to afford 3-[(3-chloro-2-methoxyphenyl)amino]-2-(2-{[3-(methanesulfonylmethyl)-1,2,3-triazol-4-yl]amino}pyrimidin-4-yl)-1H,5H,6H,7H-pyrrolo[3,2-c]pyridin-4-one (4.5 mg, 5.81%) as a yellow solid.

LC-MS: (M+H)⁺ found: 544.05.

¹H NMR (400 MHz, DMSO-d6) δ 11.49 (s, 1H), 10.19 (s, 1H), 8.43 (s, 1H), 8.29 (d, J=5.3 Hz, 1H), 7.78 (s, 1H), 7.24 (s, 1H), 6.85-6.68 (m, 3H), 6.31 (dd, J=7.5, 2.2 Hz, 1H), 5.97 (s, 2H), 3.91 (s, 3H), 3.43 (td, J=6.8, 2.4 Hz, 2H), 3.13 (s, 3H), 2.93 (t, J=6.8 Hz, 2H).

### Example 259. 3-[(3-chloro-2-methoxyphenyl)amino]-2-{2-[(3-ethyl-1,2,3-triazol-4-yl)amino]pyrimidin-4-yl}-1H,5H,6H,7H-pyrrolo[3,2-c]pyridin-4-one (compound 538)

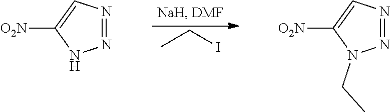

To a stirred solution of 4-nitro-3H-1,2,3-triazole (1.0 g, 8.76 mmol, 1.00 equiv) in dimethylformamide (10 mL) was added NaH (0.37 g, 9.20 mmol, 1.05 equiv, 60%) in portions at 0 degrees C. under argon atmosphere. The resulting mixture was stirred for 30 min at 0 degrees C. under argon atmosphere. To the above mixture was added ethyl iodide (1.78 g, 11.39 mmol, 1.3 equiv) in portions at 0 degrees C. The resulting mixture was stirred for additional 1 h at room temperature. The reaction was quenched by the addition of Water (10 mL) at 0 degrees C. The resulting mixture was extracted with EtOAc (3×100 mL). The combined organic layers were washed with water (3×100 mL), dried over anhydrous Na₂SO₄. After filtration, the filtrate was concentrated under reduced pressure. The residue was purified by silica gel column chromatography, eluted with PE/EtOAc (1:1) to afford 1-ethyl-5-nitro-1,2,3-triazole (700 mg, 56.18%) as a yellow oil.

¹H NMR (400 MHz, DMSO-d6) δ 8.71 (s, 1H), 4.59 (q, J=7.3 Hz, 2H), 1.51 (t, J=7.3 Hz, 3H).

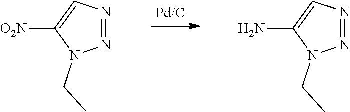

To a stirred solution of 1-ethyl-5-nitro-1,2,3-triazole (300 mg, 2.11 mmol, 1.00 equiv) in EtOH (10 mL) was added Pd/C (100 mg, 0.09 mmol, 0.04 equiv, 10%) in portions at room temperature under argon atmosphere. The resulting mixture was stirred for overnight at room temperature under hydrogen atmosphere. The resulting mixture was filtered, the filter cake was washed with EtOH (3×50 mL). The filtrate was concentrated under reduced pressure. The residue was purified by silica gel column chromatography, eluted with CH₂Cl₂/MeOH (10:1) to afford 3-ethyl-1,2,3-triazol-4-amine (170 mg, 71.82%) as a yellow oil.

LC-MS: (M+H)⁺ found: 112.95.

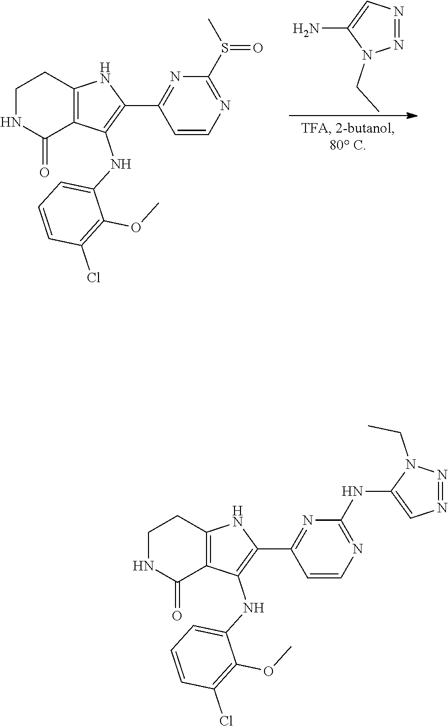

To a stirred mixture of 3-ethyl-1,2,3-triazol-4-amine (64 mg, 0.58 mmol, 5 equiv) and 3-[(3-chloro-2-methoxyphenyl)amino]-2-(2-methanesulfinylpyrimidin-4-yl)-1H,5H,6H,7H-pyrrolo[3,2-c]pyridin-4-one (50 mg, 0.116 mmol, 1.00 equiv) in butan-2-ol (2 mL) were added TFA (52 mg, 0.46 mmol, 4 equiv) in portions at room temperature under nitrogen atmosphere. The resulting mixture was stirred for 72 h at 80 degrees C. under nitrogen atmosphere. The resulting mixture was concentrated under vacuum and dissolved in DMSO. The crude product (100 mg) was purified by Prep-HPLC with the following conditions (Column: XBridge Shield RP18 OBD Column, 19*250 mm, 10 μm; Mobile Phase A: Water (10 mmol/L NH4HCO3), Mobile Phase B: MeOH-Preparative; Flow rate: 25 mL/min; Gradient: 61% B to 72% B in 11 min, 72% B; Wave Length: 254 nm; RT1 (min): 9.48) to afford 3-[(3-chloro-2-methoxyphenyl)amino]-2-{2-[(3-ethyl-1,2,3-triazol-4-yl)amino]pyrimidin-4-yl}-1H,5H,6H,7H-pyrrolo[3,2-c]pyridin-4-one (5.4 mg, 9.72%) as a white solid.

LC-MS: (M+H)⁺ found: 479.90.

¹H NMR (400 MHz, DMSO-d₆) δ 11.55 (s, 1H), 9.67 (s, 1H), 8.33-8.17 (m, 2H), 7.81 (s, 1H), 7.21 (s, 1H), 6.83-6.65 (m, 2H), 6.70 (d, J=5.3 Hz, 1H), 6.32 (m, J=7.7, 2.0 Hz, 1H), 4.36 (q, J=7.3 Hz, 2H), 3.91 (s, 3H), 3.42 (m, J=6.8, 2.6 Hz, 2H), 2.90 (t, J=6.8 Hz, 2H), 1.46 (t, J=7.3 Hz, 3H).

### Example 260. 3-[(3-chloro-2-methoxyphenyl)amino]-2-(2-{[1-(2-hydroxy-2-methylpropyl)pyrazol-3-yl]amino}pyrimidin-4-yl)-1H,5H,6H,7H-pyrrolo[3,2-c]pyridin-4-one (compound 537)

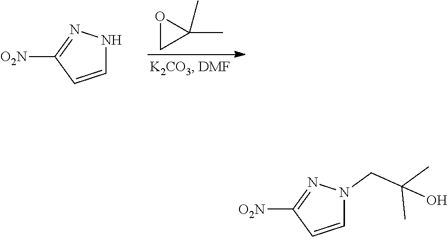

To a stirred mixture of 3-nitro-1H-pyrazole (2 g, 17.69 mmol, 1.00 equiv) and 2,2-dimethyloxirane (2.55 g, 35.37 mmol, 2 equiv) in DMF (20 mL) was added K₂CO₃ (3.67 g, 26.53 mmol, 1.5 equiv) in portions at room temperature under nitrogen atmosphere. The resulting mixture was stirred for 1 h at 100 degrees C. under nitrogen atmosphere. The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by Prep-TLC (PE/EA 3:1) to afford 2-methyl-1-(3-nitropyrazol-1-yl)propan-2-ol (3.1 g, 94.65%) as a colorless oil.

LC-MS: (M+H)⁺ found: 186.

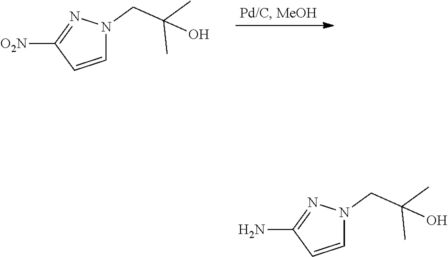

To a solution of 2-methyl-1-(3-nitropyrazol-1-yl)propan-2-ol (1 g, 5.40 mmol, 1.00 equiv) in 5 mL MeOH was added Pd/C (10%, 100 mg) in a pressure tank. The mixture was hydrogenated at room temperature under 30 psi of hydrogen pressure for 1 h, filtered through a Celite pad and concentrated under reduced pressure. The residue was purified by reverse flash chromatography with the following conditions: column, silica gel; mobile phase, MeCN in water, 10% to 50% gradient in 10 min; detector, UV 254 nm to afford 1-(3-aminopyrazol-1-yl)-2-methylpropan-2-ol (250 mg, 20.88%) as a off-white solid.

LC-MS: (M+H)⁺ found: 156.

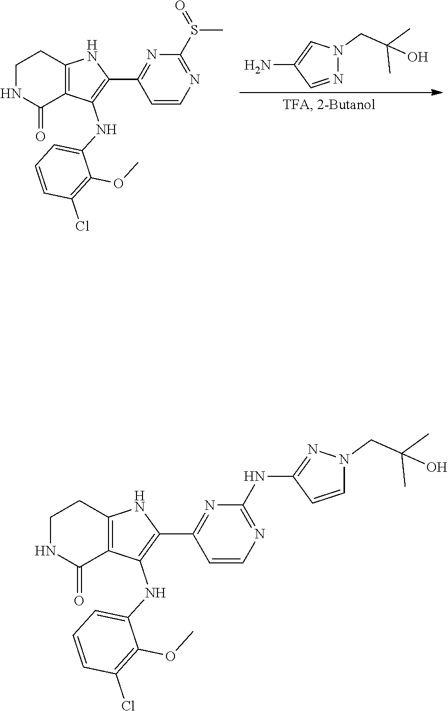 [SMILES: COc1c(Cl)cccc1Nc1c(-c2ccnc(S(C)=O)n2)[nH]c2c1C(=O)NCC2 ; CC(C)(O)Cn1cc(N)cn1 ; COc1c(Cl)cccc1Nc1c(-c2ccnc(Nc3ccn(CC(C)(C)O)n3)n2)[nH]c2c1C(=O)NCC2]

To a stirred mixture of 3-[(3-chloro-2-methoxyphenyl)amino]-2-(2-methanesulfinylpyrimidin-4-yl)-1H,5H,6H,7H-pyrrolo[3,2-c]pyridin-4-one (80 mg, 0.19 mmol, 1.00 equiv) and 1-(3-aminopyrazol-1-yl)-2-methylpropan-2-ol (144 mg, 0.93 mmol, 5 equiv) in butan-2-ol (4 mL) was added TFA (42 mg, 0.37 mmol, 2 equiv) in portions at room temperature under nitrogen atmosphere. The resulting mixture was stirred for 3 days at 80 degrees C. under nitrogen atmosphere. The resulting mixture was concentrated under reduced pressure. The crude product (80 mg) was purified by Prep-HPLC with the following conditions (Column: YMC-Actus Triart C18 ExRS, 30*150 mm, 5 μm; Mobile Phase A: Water (10 mmol/L NH4HCO3), Mobile Phase B: ACN; Flow rate: 60 mL/min; Gradient: 23% B to 50% B in 8 min, 50% B; Wave Length: 254/220 nm; RT1 (min): 5.7) to afford 3-[(3-chloro-2-methoxyphenyl)amino]-2-(2-{[1-(2-hydroxy-2-methylpropyl)pyrazo]-3-yl]amino}pyrimidin-4-yl)-1H,5H,6H,7H-pyrrolo[3,2-c]pyridin-4-one (14.4 mg, 14.79%) as a yellow solid.

LC-MS: (M+H)⁺ found: 523.30.

¹H NMR (300 MHz, DMSO-d6) δ 11.46 (s, 1H), 9.13 (s, 1H), 8.20 (d, J=5.3 Hz, 1H), 7.82 (s, 1H), 7.52 (d, J=2.3 Hz, 1H), 7.15 (d, J=2.7 Hz, 1H), 6.85-6.70 (m, 2H), 6.70-6.59 (m, 2H), 6.37-6.30 (m, 1H), 4.71 (s, 1H), 3.88 (d, J=8.3 Hz, 5H), 3.39 (d, J=2.3 Hz, 2H), 2.87 (t, J=6.8 Hz, 2H), 1.06 (s, 6H).

### Example 261. 2-{[2,4′-bipyrimidin]-4-yl}-3-[(3-chloro-2-methoxyphenyl)amino]-1H,5H,6H,7H-pyrrolo[3,2-c]pyridin-4-one (compound 546)

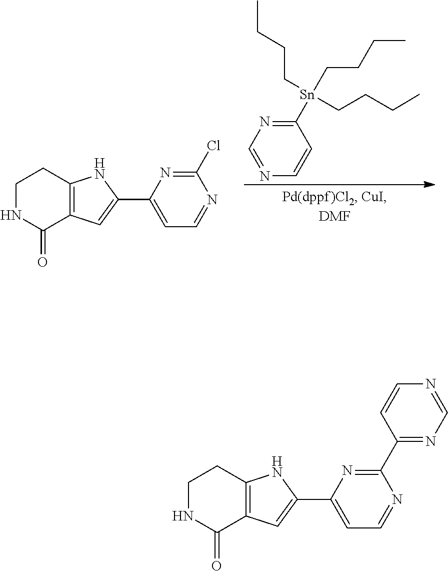

To a stirred solution of 2-(2-chloropyrimidin-4-yl)-1H,5H,6H,7H-pyrrolo[3,2-c]pyridin-4-one (200 mg, 0.804 mmol, 1.00 equiv), Pd(dppf)Cl₂CH₂Cl₂ (65.52 mg, 0.080 mmol, 0.1 equiv) and CuI (15.32 mg, 0.080 mmol, 0.1 equiv) in DMF (2 mL) was added 4-(tributylstannyl)pyrimidine (326.58 mg, 0.884 mmol, 1.1 equiv) at room temperature under nitrogen atmosphere. The resulting mixture was stirred for 1 h at 130 room temperature under nitrogen atmosphere. The resulting mixture was concentrated under vacuum. The residue was purified by silica gel column chromatography, eluted with CH₂Cl₂/MeOH (20:1) to afford 2-([2,4′-bipyrimidin]-4-yl)-1H,5H,6H,7H-pyrrolo[3,2-c]pyridin-4-one (120 mg, 51.04%) as a yellow solid.

LC-MS: (M+H)⁺ found: 292.95.

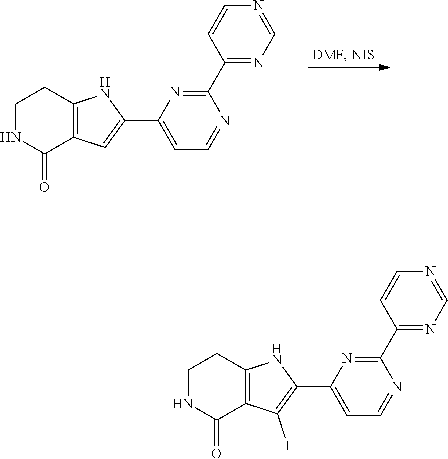 [SMILES: O=C1NCCc2[nH]c(-c3ccnc(-c4ccncn4)n3)cc21 ; O=C1NCCc2[nH]c(-c3ccnc(-c4ccncn4)n3)c(I)c21]

To a stirred solution of 2-{[2,4′-bipyrimidin]-4-yl}-1H,5H,6H,7H-pyrrolo[3,2-c]pyridin-4-one (120.00 mg, 0.411 mmol, 1 equiv) and NIS (92.36 mg, 0.411 mmol, 1 equiv) in DMF (2 mL) at room temperature under nitrogen atmosphere. The resulting mixture was stirred for 1 h at 30° C. under nitrogen atmosphere. The reaction was quenched by the addition of sat. Sodium sulfite (aq.) (0.5 mL) at 0° C. The resulting mixture was concentrated under vacuum. The residue was purified by silica gel column chromatography, eluted with CH₂Cl₂/MeOH (10:1) to afford 2-{[2,4′-bipyrimidin]-4-yl}-3-iodo-1H,5H,6H,7H-pyrrolo[3,2-c]pyridin-4-one (150.00 mg, 87.37%) as a brown solid.

LC-MS: (M+H)⁺ found: 419.00.

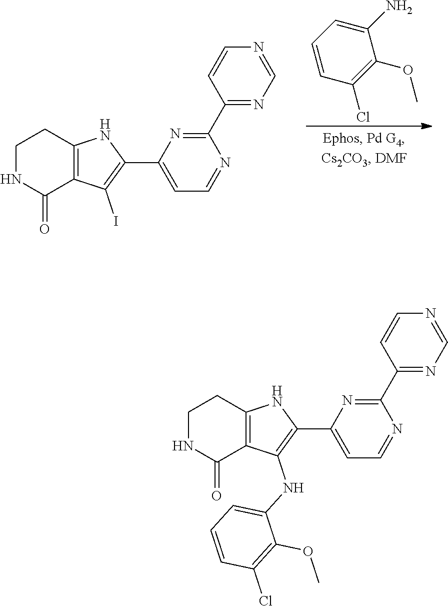

To a stirred mixture of 2-{[2,4′-bipyrimidin]-4-yl}-3-iodo-1H,5H,6H,7H-pyrrolo[3,2-c]pyridin-4-one (100 mg, 0.239 mmol, 1.00 equiv) and, 3-chloro-2-methoxyaniline (37.69 mg, 0.239 mmol, 1 equiv) in DMF (1.5 mL) were added EPhos Pd G4 (65.89 mg, 0.072 mmol, 0.3 equiv) and Cs₂CO₃ (155.82 mg, 0.478 mmol, 2 equiv) under argon atmosphere. The resulting suspension was backfilled with argon three times and stirred for 3 h at 50° C. LCMS confirmed completion of reaction and desired product was observed. The resulting mixture was filtered through a pad of silica and the filter cake was washed with DCM (2×10 mL). The filtrate was concentrated under reduced pressure. The crude product (50.00 mg) was purified by Prep-HPLC with the following conditions (Column: XBridge Prep C18 OBD Column, 30*100 mm, 5pim; Mobile Phase A: Water (10 mmol/L NH4HCO3), Mobile Phase B: ACN; Flow rate: 60 mL/min; Gradient: 22% B to 50% B in 8 min, 50% B; Wave Length: 254/220 nm; RT1 (min): 7.53) to afford 2-{[2,4′-bipyrimidin]-4-yl}-3-[(3-chloro-2-methoxyphenyl)amino]-1H,5H,6H,7H-pyrrolo[3,2-c]pyridin-4-one (13.1 mg, 12.21%) as a yellow solid.

LC-MS: (M+H)⁺ found: 448.20.

¹H NMR (400 MHz, DMSO-d6) δ 12.06 (s, 1H), 9.37 (d, J=1.4 Hz, 1H), 9.02 (d, J=5.2 Hz, 1H), 8.76 (d, J=5.5 Hz, 1H), 8.48 (d, J=5.3 Hz, 1H), 7.95 (s, 1H), 7.29-7.23 (m, 2H), 6.84-6.75 (m, 2H), 6.34 (d, J=5.8, 1H), 3.93 (s, 3H), 3.45 (t, J=7.8, 3.9 Hz, 2H), 2.95 (t, J=6.8 Hz, 2H).

### Example 262. 3-[(3-chloro-2-methoxyphenyl)amino]-2-[2-(1-methyl-1,2,3-triazol-4-yl)pyrimidin-4-yl]-1H,5H,6H,7H-pyrrolo[3,2-c]pyridin-4-one (compound 545)

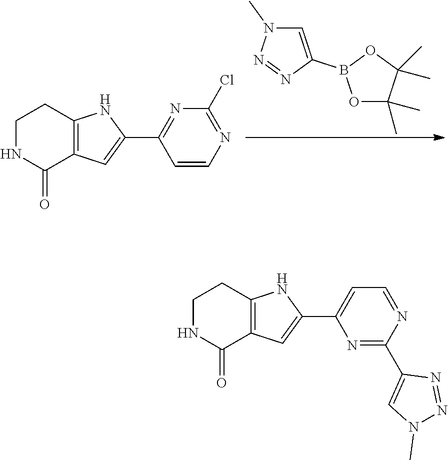

To a stirred solution of 2-(2-chloropyrimidin-4-yl)-1H,5H,6H,7H-pyrrolo[3,2-c]pyridin-4-one (100.00 mg, 0.402 mmol, 1.00 equiv) and 1-methyl-4-(4,4,5,5-tetramethyl-1,3,2-dioxaborolan-2-yl)-1,2,3-triazole (100.89 mg, 0.482 mmol, 1.2 equiv) in Toluene (1 mL) and H₂O (0.1 mL) was added Pd(dppf)Cl₂CH₂Cl₂ (32.76 mg, 0.040 mmol, 0.1 equiv) at room temperature under argon atmosphere. The resulting mixture was stirred overnight at 50° C. under argon atmosphere. The resulting mixture was concentrated under vacuum. The residue was purified by silica gel column chromatography, eluted with CH₂Cl₂/MeOH (20:1) to afford 2-[2-(1-methyl-1,2,3-triazol-4-yl)pyrimidin-4-yl]-1H,5H,6H,7H-pyrrolo[3,2-c]pyridin-4-one (60.00 mg, 50.52%) as a yellow solid.

LC-MS: (M+H)⁺ found: 296.2.

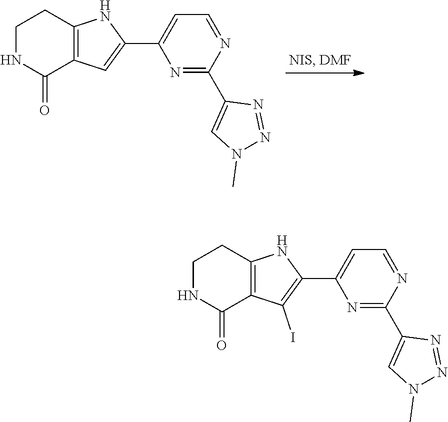 [SMILES: Cn1cc(-c2nccc(-c3cc4c([nH]3)CCNC4=O)n2)nn1 ; Cn1cc(-c2nccc(-c3[nH]c4c(c3I)C(=O)NCC4)n2)nn1]

To a stirred solution of 2-[2-(1-methyl-1,2,3-triazol-4-yl)pyrimidin-4-yl]-1H,5H,6H,7H-pyrrolo[3,2-c]pyridin-4-one (50.00 mg, 0.169 mmol, 1 equiv) in DMF (1 mL) was added NIS (38.09 mg, 0.169 mmol, 1 equiv) in portions at 0° C. under nitrogen atmosphere. The resulting mixture was stirred for 1 h at 40° C. under nitrogen atmosphere. The resulting mixture was concentrated under vacuum. The residue was purified by silica gel column chromatography, eluted with CH₂Cl₂/MeOH (30:1) to afford 3-iodo-2-[2-(1-methyl-1,2,3-triazol-4-yl)pyrimidin-4-yl]-1H,5H,6H,7H-pyrrolo[3,2-c]pyridin-4-one (70.00 mg, 98.15%) as a brown solid.

LC-MS: (M+H)⁺ found: 421.90.

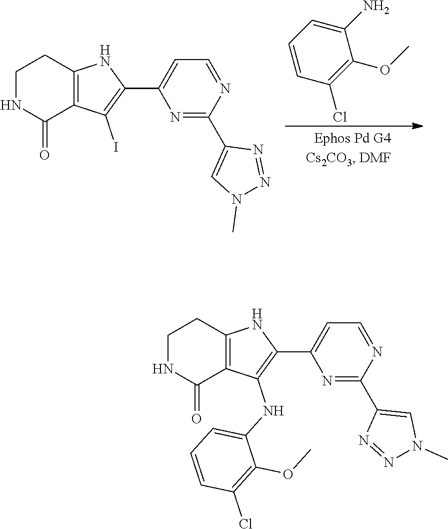

To a stirred mixture of 3-iodo-2-[2-(1-methyl-1,2,3-triazol-4-yl)pyrimidin-4-yl]-1H,5H,6H,7H-pyrrolo[3,2-c]pyridin-4-one (60 mg, 0.142 mmol, 1.00 equiv) and, 3-chloro-2-methoxyaniline (22.45 mg, 0.142 mmol, 1.00 equiv) in DMF (2 mL) were added EPhos Pd G4 (65.42 mg, 0.071 mmol, 0.50 equiv) and Cs₂CO₃ (92.83 mg, 0.284 mmol, 2.00 equiv) under argon atmosphere. The resulting suspension was backfilled with argon three times and stirred for 3 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford crude 3-[(3-chloro-2-methoxyphenyl)amino]-2-[2-(1-methyl-1,2,3-triazol-4-yl)pyrimidin-4-yl]-1H,5H,6H,7H-pyrrolo[3,2-c]pyridin-4-one (40 mg) as a yellow oil. The crude product (40.00 mg) was purified by Prep-HPLC with the following conditions (Column. XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 20% B to 49% B in 8 min, 49% B; Wave Length: 254/220 nm; RT1 (min): 6.32) to afford 3-[(3-chloro-2-methoxyphenyl)amino]-2-[2-(1-methyl-1,2,3-triazol-4-yl)pyrimidin-4-yl]-1H,5H,6H,7H-pyrrolo[3,2-c]pyridin-4-one (2.8 mg, 4.21%) as a yellow solid. LC-MS: (M+H)⁺ found: 451.05.

¹H NMR (400 MHz, Chloroform-d) δ 10.97 (s, 1H), 8.43 (d, J=5.5 Hz, 1H), 8.26 (s, 1H), 7.54 (s, 1H), 6.88 (d, J=5.5 Hz, 1H), 6.84-6.68 (m, 2H), 6.38-6.32 (m, 1H), 5.33 (s, 1H), 4.22 (s, 3H), 4.09 (s, 3H), 3.64-3.60 (m, 2H), 3.01 (t, J=6.8 Hz, 2H).

### Example 263. 3-[(3-chloro-2-methoxyphenyl)amino]-2-[2-(1-methylpyrazol-3-yl)pyrimidin-4-yl]-1H,5H,6H,7H-pyrrolo[3,2-c]pyridin-4-one (compound 544)

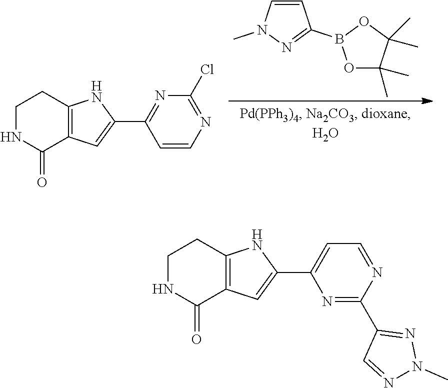

To a stirred mixture of 2-(2-chloropyrimidin-4-yl)-1H,5H,6H,7H-pyrrolo[3,2-c]pyridin-4-one (100.00 mg, 0.402 mmol, 1.00 equiv) and 1-methyl-3-(4,4,5,5-tetramethyl-1,3,2-dioxaborolan-2-yl)pyrazole (100.41 mg, 0.482 mmol, 1.20 equiv) in dioxane (4.00 mL) and H2O (0.80 mL) were added Pd(PPh₃)₄ (46.47 mg, 0.040 mmol, 0.10 equiv) and Na₂CO₃ (85.24 mg, 0.804 mmol, 2.00 equiv) in portions at room temperature under nitrogen atmosphere. The resulting mixture was stirred for 1 h at 90 degrees C. under nitrogen atmosphere. The reaction was monitored by LCMS. The resulting mixture was concentrated under reduced pressure. The residue was purified by Prep-TLC (CH₂Cl₂/MeOH 10:1) to afford 2-[2-(1-methylpyrazol-3-yl)pyrimidin-4-yl]-1H,5H,6H,7H-pyrrolo[3,2-c]pyridin-4-one (100 mg, 83.65%) as a yellow solid.

LC-MS: (M+H)⁺ found: 294.95.

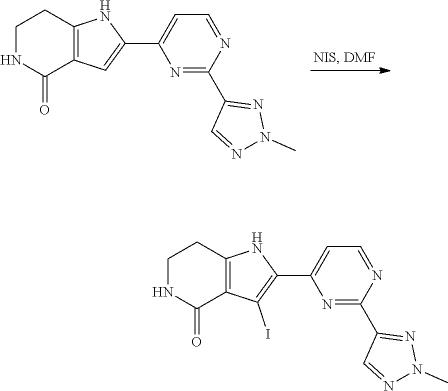 [SMILES: Cn1ncc(-c2nccc(-c3cc4c([nH]3)CCNC4=O)n2)n1 ; Cn1ncc(-c2nccc(-c3[nH]c4c(c3I)C(=O)NCC4)n2)n1]

To a stirred mixture of 2-[2-(1-methylpyrazol-3-yl)pyrimidin-4-yl]-1H,5H,6H,7H-pyrrolo[3,2-c]pyridin-4-one (100.00 mg, 0.340 mmol, 1.00 equiv) in DMF (3 mL) was added NIS (76.44 mg, 0.340 mmol, 1.00 equiv) in portions at 0 degrees C. under air atmosphere. The resulting mixture was stirred for 4 h at room temperature under air atmosphere. The reaction was monitored by LCMS. The reaction was quenched with sat. sodium hyposulfite (aq.) at 0 degrees C. The precipitated solids were collected by filtration and washed with water (3×10 mL) to afford 3-iodo-2-[2-(I-methylpyrazol-3-yl)pyrimidin-4-yl]-1H,5H,6H,7H-pyrrolo[3,2-c]pyridin-4-one (105.00 mg, 69.86%) as a yellow solid.

LC-MS: (M+H)⁺ found: 420.85.

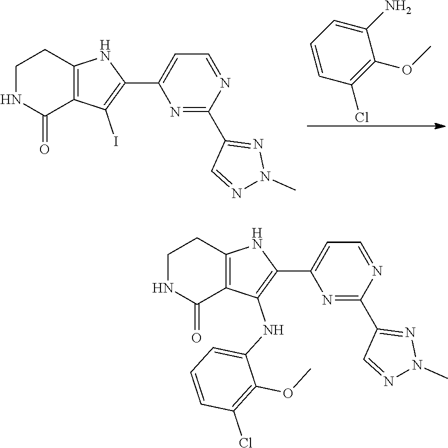

To a stirred mixture of 3-iodo-2-[2-(1-methylpyrazol-3-yl)pyrimidin-4-yl]-1H,5H,6H,7H-pyrrolo[3,2-c]pyridin-4-one (100.00 mg, 0.238 mmol, 1.00 equiv) and 3-chloro-2-methoxyaniline (37.50 mg, 0.238 mmol, 1.00 equiv) in DMF (2.00 mL) were added EPhos Pd G4 (12.73 mg, 0.024 mmol, 0.10 equiv) and Cs₂CO₃ (155.07 mg, 0.476 mmol, 2.00 equiv) in portions at room temperature under argon atmosphere. The resulting mixture was stirred for 2 h at 50 degrees C. under argon atmosphere. The reaction was monitored by LCMS. Desired product could be detected by LCMS. The resulting mixture was concentrated under reduced pressure. The residue was purified by silica gel column chromatography, eluted with CH₂Cl₂/MeOH (20:1) to afford 3-[(3-chloro-2-methoxyphenyl)amino]-2-[2-(1-methylpyrazol-3-yl)pyrimidin-4-yl]-1H,5H,6H,7H-pyrrolo[3,2-c]pyridin-4-one (50.0 mg) as a light yellow solid. The crude product (50 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 28% B to 58% B in 7 min, 58% B; Wave Length: 254/220 nm; RT1 (min): 5.92) to afford 3-[(3-chloro-2-methoxyphenyl)amino]-2-[2-(1-methylpyrazol-3-yl)pyrimidin-4-yl]-1H,5H,6H,7H-pyrrolo[3,2-c]pyridin-4-one (38.0 mg, 35.28%) as a off-white solid.

LC-MS: (M+H)⁺ found: 451.00.

¹H NMR (300 MHz, DMSO-d6) δ 11.86 (s, 1H), 8.55 (d, J=5.4 Hz, 1H), 7.92 (s, 1H), 7.80 (d, J=2.2 Hz, 1H), 7.21 (d, J=2.7 Hz, 1H), 7.12-7.02 (m, 2H), 6.87-6.75 (m, 2H), 6.37 (dd, J=6.1, 3.6 Hz, 1H), 3.93 (d, J=6.8 Hz, 6H), 3.43 (td, J=6.5, 2.4 Hz, 2H), 2.93 (t, J=6.7 Hz, 2H).

### Example 264. 3-[(3-chloro-2-methoxyphenyl)amino]-2-[2-(2-methyl-1,2,3-triazol-4-yl)pyrimidin-4-yl]-1H,5H,6H,7H-pyrrolo[3,2-c]pyridin-4-one (compound 543)

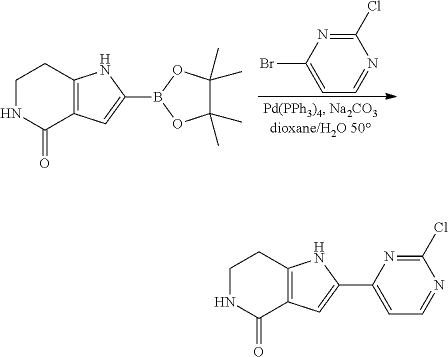

To a stirred solution of 2-(4,4,5,5-tetramethyl-1,3,2-dioxaborolan-2-yl)-1H,5H,6H,7H-pyrrolo[3,2-c]pyridin-4-one (1626.14 mg, 6.204 mmol, 1.2 equiv) and 4-bromo-2-chloropyrimidine (1000 mg, 5.170 mmol, 1 equiv) in 1,4-dioxane (5 mL) and H₂O (1 mL) was added Pd(PPh₃)₄ (1194.84 mg, 1.034 mmol, 0.2 equiv) and Na2CO3 (1095.88 mg, 10.340 mmol, 2 equiv) in portions at room temperature under nitrogen atmosphere. The resulting mixture was stirred for overnight at 50 degrees C. under nitrogen atmosphere. The resulting mixture was diluted with water (10 mL). The precipitated solids were collected by filtration and washed with EtOAc (3×5 mL). The residue was purified by silica gel column chromatography, eluted with CH₂Cl2/MeOH (10:1) to afford 2-(2-chloropyimidin-4-yl)-1H,5H,6H,7H-pyrrolo[3,2-c]pyridin-4-one (852 mg, 66.27%) as a light yellow solid.

LC-MS: (M+H)⁺ found: 249.2.

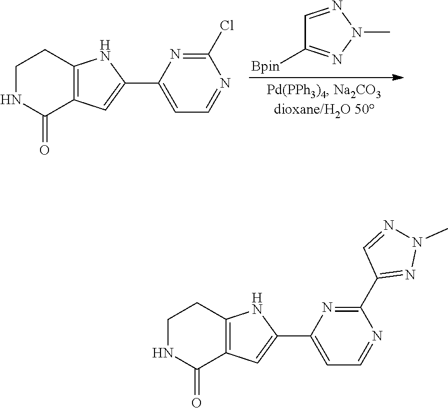

To a stirred mixture of 2-(2-chloropyrimidin-4-yl)-1H,5H,6H,7H-pyrolo[3,2-c]pyridin-4-one (200 mg, 0.80 mmol, 1 equiv) and 2-methyl-4-(4,4,5,5-tetramethyl-1,3,2-dioxaborolan-2-yl)-1,2,3-triazole (201.8 mg, 0.97 mmol, 1.2 equiv) in 1,4-dioxane (5 mL, 0.06 mmol) and H₂O (1 mL) was added Pd(PPh₃)₄ (185.9 mg, 0.16 mmol, 0.2 equiv) and Na₂CO₃ (170.5 mg, 1.61 mmol, 2 equiv) at room temperature under argon atmosphere. The resulting mixture was stirred for overnight at 50° C. under argon atmosphere. The resulting mixture was concentrated under reduced pressure. The aqueous layer was extracted with EtOAc (3×20 mL). The combined organic layers were dried over anhydrous Na₂SO₄. After filtration, the filtrate was concentrated under reduced pressure. The residue was purified by silica gel column chromatography, eluted with CH₂Cl₂/MeOH (20:1) to afford 2-[2-(2-methyl-1,2,3-triazol-4-yl)pyrimidin-4-yl]-1H,5H,6H,7H-pyrrolo[3,2-c]pyridin-4-one (150 mg, 63.16%) as a yellow solid.

LC-MS: (M+H)⁺ found: 296.

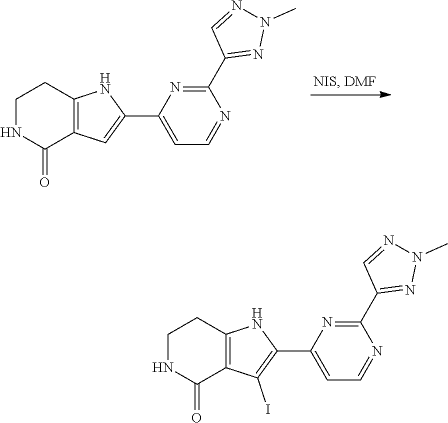 [SMILES: Cn1ncc(-c2nccc(-c3cc4c([nH]3)CCNC4=O)n2)n1 ; Cn1ncc(-c2nccc(-c3[nH]c4c(c3I)C(=O)NCC4)n2)n1]

To a stirred mixture of 2-[2-(2-methyl-1,2,3-triazol-4-yl)pyrimidin-4-yl]-1H,5H,6H,7H-pyrrolo[3,2-c]pyridin-4-one (150 mg, 0.51 mmol, 1 equiv) in DMF (5 mL) was added NIS (137.14 mg, 0.61 mmol, 1.2 equiv) in portions at room temperature under argon atmosphere. The resulting mixture was stirred for 3 h at room temperature under argon atmosphere. The reaction was quenched with sat. sodium sulfite (aq.) at room temperature. The precipitated solids were collected by filtration and washed with water (3×10 mL). The residue was purified by silica gel column chromatography, eluted with CH₂Cl2/MeOH (10:1) to afford 3-iodo-2-[2-(2-methyl-1,2,3-triazol-4-yl)pyrimidin-4-yl]-1H,5H,6H,7H-pyrrolo[3,2-c]pyridin-4-one as a light yellow solid.

LC-MS: (M+H)⁺ found: 421.9.

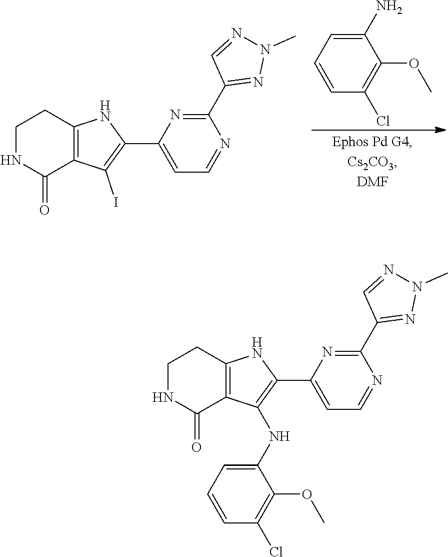

To a stirred mixture of 3-iodo-2-[2-(2-methyl-1,2,3-triazol-4-yl)pyrimidin-4-yl]-1H,5H,6H,7H-pyrrolo[3,2-c]pyridin-4-one (100 mg, 0.22 mmol, 1 equiv) and 3-chloro-2-methoxyaniline (36.14 mg, 0.23 mmol, 1.05 equiv) in DMF (2 mL) was added EPhos Pd G4 (20.06 mg, 0.02 mmol, 0.1 equiv) and Cs₂CO₃ (142.33 mg, 0.44 mmol, 2 equiv) at room temperature under argon atmosphere. The resulting mixture was stirred for 3 h at 50° C. under argon atmosphere. The resulting mixture was concentrated under vacuum. The crude product (100 mg) was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 26% B to 50% B in 9 min, 50%/B; Wave Length: 254/220 nm; RT1 (min): 8.85) to afford 3-[(3-chloro-2-methoxyphenyl)amino]-2-[2-(2-methyl-1,2,3-triazol-4-yl)pyrimidin-4-yl]-1H,5H,6H,7H-pyrrolo[3,2-c]pyridin-4-one (16.5 mg, 16.45%) as a light yellow solid.

LC-MS: (M+H)⁺ found: 451.00.

¹H NMR (400 MHz, DMSO-d6) δ 11.93 (s, 1H), 8.61 (d, J=5.5 Hz, 1H), 8.40 (s, 1H), 7.94 (s, 1H), 7.25 (t, J=2.7 Hz, 1H), 7.11 (d, J=5.5 Hz, 1H), 6.88-6.72 (m, 2H), 6.46-6.26 (m, 1H), 4.25 (s, 3H), 3.92 (s, 2H), 3.44 (td, J=6.8, 2.5 Hz, 2H), 2.94 (t, J=6.7 Hz, 2H).

### Example 265. 3-[(3-chloro-2-methoxyphenyl)amino]-2-[2-(pyridazin-4-yl)pyrimidin-4-yl]-1H,5H,6H,7H-pyrrolo[3,2-c]pyridin-4-one (compound 542)

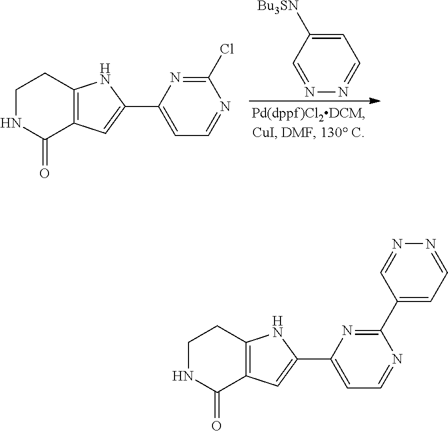

To a solution of 2-(2-chloropyrimidin-4-yl)-1H,5H,6H,7H-pyrrolo[3,2-c]pyridin-4-one (150 mg, 0.60 mmol, 1.00 equiv), CuI (11 mg, 0.06 mmol, 0.10 equiv) and Pd(dppf)Cl₂CH₂Cl₂ (49 mg, 0.06 mmol, 0.10 equiv) in DMF (2.00 mL) were added 4-(tributylstannyl)pyridazine (240 mg, 0.65 mmol, 1.08 equiv) dropwise at room temperature under nitrogen atmosphere. The resulting mixture was stirred for 1 h at 130 degrees C. under nitrogen atmosphere. The reaction was quenched by the addition of KF (sat.) (5.00 mL) at room temperature. The resulting mixture was concentrated under reduced pressure. The residue was purified by silica gel column chromatography, eluted with CH₂Cl₂/MeOH (10:1) to afford 2-[2-(pyridazin-4-yl)pyrimidin-4-yl]-1H,5H,6H,7H-pyrrolo[3,2-c]pyridin-4-one (110 mg, 62.39%) as a yellow solid.

LC-MS: (M+H)⁺ found: 293.2.

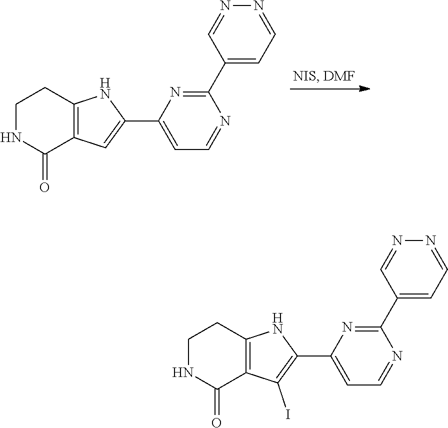 [SMILES: O=C1NCCc2[nH]c(-c3ccnc(-c4ccnnc4)n3)cc21 ; O=C1NCCc2[nH]c(-c3ccnc(-c4ccnnc4)n3)c(I)c21]

To a stirred solution of 2-[2-(pyridazin-4-yl)pyrimidin-4-yl]-1H,5H,6H,7H-pyrrolo[3,2-c]pyridin-4-one (90 mg, 0.31 mmol, 1.00 equiv) in DMF (2.00 mL) was added NIS (69 mg, 0.31 mmol, 1.00 equiv) dropwise at 0° C. under nitrogen atmosphere. The resulting mixture was stirred for 2 h at room temperature under nitrogen atmosphere. The reaction was quenched by the addition of Na2SO3 (sat.) (2.00 mL) at room temperature. The resulting mixture was concentrated under reduced pressure. The residue was purified by silica gel column chromatography, eluted with CH₂Cl₂/MeOH (10:1) to afford 3-iodo-2-[2-(pyridazin-4-yl)pyrimidin-4-yl]-1H,5H,6H,7H-pyrrolo[3,2-c]pyridin-4-one (70 mg, 54.36%) as a yellow solid.

LC-MS: (M+H)⁺ found: 418.85.

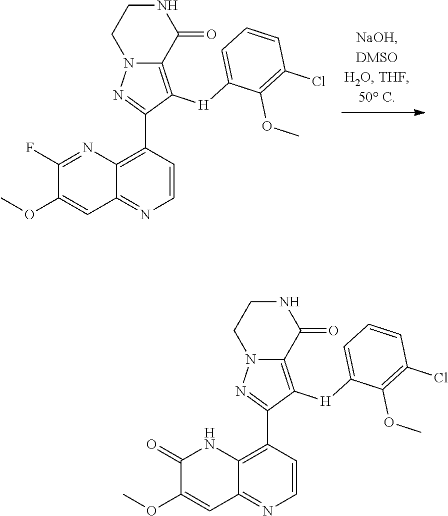

To a stirred solution of 3-iodo-2-[2-(pyridazin-4-yl)pyrimidin-4-yl]-1H,5H,6H,7H-pyrrolo[3,2-c]pyridin-4-one (60 mg, 0.143 mmol, 1.00 equiv) Cs₂CO₃ (93.49 mg, 0.286 mmol, 2 equiv) and EPhos Pd G4 (39.54 mg, 0.043 mmol, 0.3 equiv) in DMF (2 mL) was added 3-chloro-2-methoxyaniline (20.35 mg, 0.129 mmol, 0.9 equiv) dropwise at room temperature under argon atmosphere. The resulting mixture was stirred for 3 h at 50° C. under argon atmosphere. The resulting mixture was concentrated under reduced pressure. The residue was purified by silica gel column chromatography, eluted with CH₂Cl₂/MeOH (10:1) to afford crude product. The crude product (30 mg) was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 23% B to 48% B in 8 min, 48% B; Wave Length: 254/220 nm; RT1 (min): 8.85) to afford 3-[(3-chloro-2-methoxyphenyl)amino]-2-[2-(pyridazin-4-yl)pyrimidin-4-yl]-1H,5H,6H,7H-pyrrolo[3,2-c]pyridin-4-one (16.5 mg, 25.14%) as a yellow solid.

LC-MS: (M+H)⁺ found: 447.90.

¹H NMR (400 MHz, DMSO-d6) δ 12.17 (s, 1H), 10.23 (s, 1H), 9.45 (d, 1H), 8.74 (d, 1H), 8.55-8.49 (m, 1H), 7.86 (s, 1H), 7.31 (s, 1H), 7.16 (d, 1H), 6.85-6.76 (m, 2H), 6.34-6.31 (m, 1H), 3.95 (s, 3H), 3.48-3.46 (m, 2H), 2.97 (t, 2H).

### Example 266. 3-[(3-fluoro-2-methoxyphenyl)amino]-2-(7-methoxy-6-{[1-(prop-2-enoyl)piperidin-4-yl]oxy}-1,5-naphthyridin-4-yl)-1H,5H,6H,7H-pyrrolo[3,2-c]pyridin-4-one (compound 554)

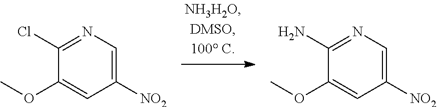

A solution of 2-chloro-3-methoxy-5-nitropyridine (6.6 g, 35.00 mmol, 1.00 equiv) in NH₃H₂O (75 mL) and DMSO (75 mL) was stirred for 8 h at 100 degrees C. The mixture was allowed to cool down to room temperature. The resulting mixture was diluted with H₂O (20 mL). The resulting mixture was extracted with EtOAc (3×100 mL). The combined organic layers were washed with brine (2×30 mL), dried over anhydrous Na₂SO₄. After filtration, the filtrate was concentrated under reduced pressure. The crude product mixture was used in the next step directly without further purification to afford 3-methoxy-5-nitropyridin-2-amine (5.6 g, 94.60%) as a yellow solid.

LC-MS: [M−H]⁻ found: 169.9.

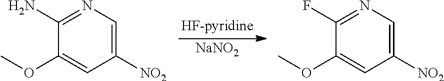

To a stirred solution of 3-methoxy-5-nitropyridin-2-amine (5.5 g, 32.5 mmol, 1 equiv) in 70% wt. HF-pyridine (110 mL) was added sodium nitrite (2.7 g, 39.1 mmol, 1.2 equiv) in portions at 0 degrees C. under nitrogen atmosphere. The resulting mixture was stirred for overnight at room temperature under nitrogen atmosphere. The reaction was quenched with ice/water at 0 degrees C. The mixture was basified to pH 9 with saturated Na2CO3 (aq.). The resulting mixture was extracted with EtOAc (3×400 mL). The combined organic layers were washed with brine (2×200 mL), dried over anhydrous Na₂SO₄. After filtration, the filtrate was concentrated under reduced pressure. The residue was purified by silica gel column chromatography, eluted with hexane/EtOAc (L: 1) to afford 2-fluoro-3-methoxy-5-nitropyridine (4.2 g, 75%) as a yellow solid.

LC-MS: [M−H]⁻ found: 173.05.

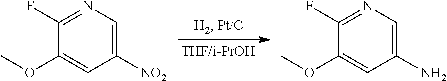

A solution of 2-fluoro-3-methoxy-5-nitropyridine (1 g, 5.81 mmol, 1.00 equiv) and Pt/C (1.13 g, 0.58 mmol, 0.1 equiv, 10%) in THF (2 mL) and i-PrOH (2 mL) was stirred for overnight at 45 degrees C. under hydrogen atmosphere. The mixture was allowed to cool down to room temperature. The resulting mixture was filtered. the filter cake was washed with DCM (2×100 mL). The filtrate was concentrated under reduced pressure. This resulted in 6-fluoro-5-methoxypyridin-3-amine (0.8 g, 96.88%) as a yellow solid.

LC-MS: [M−H]⁻ found 142.90.

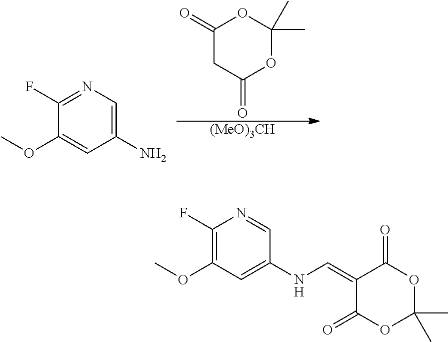

A solution of 6-fluoro-5-methoxypyridin-3-amine (3 g, 21.13 mmol, 1 equiv) and meldrum's acid (3.2 g, 21.13 mmol, 1.00 equiv) in trimethyl orthoformate (20 mL) was stirred for 2 h at 108 degrees C. under nitrogen atmosphere. The mixture was allowed to cool down to room temperature. The resulting mixture was concentrated under reduced pressure. This resulted in 5-{[(6-fluoro-5-methoxypyridin-3-yl)amino]methylidene}-2,2-dimethyl-1,3-dioxane-4,6-dione (6.2 g, 99%) as a brown solid.

LC-MS: [M+H]⁺ found: 295.1.

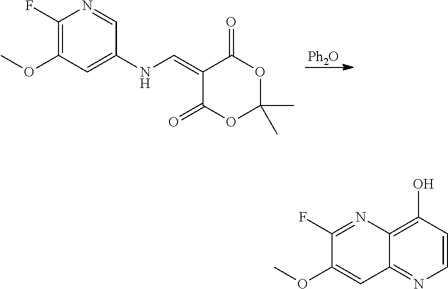

A solution of 5-{[(6-fluoro-5-methoxypyridin-3-yl)amino]methylidene}-2,2-dimethyl-1,3-dioxane-4,6-dione (6 g, 20.27 mmol, 1 equiv) in diphenyl-ether (40 mL) was stirred for 1 h at 250 degrees C. under argon atmosphere. The mixture was allowed to cool down to room temperature. The resulting mixture was diluted with Et₂O (20 mL). The precipitated solids were collected by filtration and washed with Et₂O (2×100 mL). This resulted in 6-fluoro-7-methoxy-1,5-naphthyridin-4-ol (3.8 g, 26.33%) as a brown solid.

LC-MS: [M+H] found: 195.2.

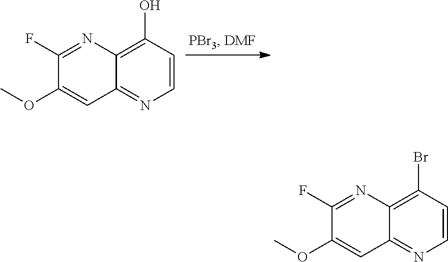

A solution of 6-fluoro-7-methoxy-1,5-naphthyridin-4-ol (1 g, 5.15 mmol, 1.00 equiv) and PBr₃ (1.39 g, 5.15 mmol, 1.00 equiv) in DMF (28.00 mL) was stirred for 2 h at 45 degrees C. under nitrogen atmosphere. The mixture was allowed to cool down to room temperature. The reaction was quenched with ice/water at 0 degrees C. The resulting mixture was extracted with EtOAc (2×20 mL). The combined organic layers were washed with brine (1×60 mL), dried over anhydrous Na₂SO₄. After filtration, the filtrate was concentrated under reduced pressure. The residue was purified by silica gel column chromatography, eluted with PE/EtOAc (1:1) to afford 8-bromo-2-fluoro-3-methoxy-1,5-naphthyridine (1.1 g, 77.6%) as a yellow solid.

LC-MS: [M+H]⁺ found: 257.1.

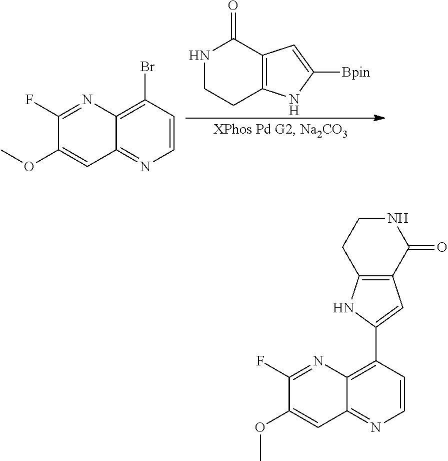

To a solution of 8-bromo-2-fluoro-3-methoxy-1,5-naphthyridine (360 mg, 1.40 mmol, 1.00 equiv) and 2-(4,4,5,5-tetramethyl-1,3,2-dioxaborolan-2-yl)-1H,5H,6H,7H-pyrrolo[3,2-c]pyridin-4-one (440 mg, 1.68 mmol, 1.20 equiv) in 1,4-dioxane (4.00 mL) and water (1.00 mL) were added sodium methaneperoxoate sodium (299 mg, 2.80 mmol, 2.00 equiv) and 2nd Generation XPhos Precatalyst (110 mg, 0.14 mmol, 0.10 equiv). After stirring for 4 h at 50 degrees C. under a nitrogen atmosphere, the resulting mixture was concentrated under reduced pressure. The residue was purified by silica gel column chromatography, eluted with CH₂Cl₂/MeOH (10:1) to afford 2-(6-fluoro-7-methoxy-1,5-naphthyridin-4-yl)-1H,5H,6H,7H-pyrrolo[3,2-c]pyridin-4-one (250 mg, 57.16%) as a yellow solid.

LC-MS: [M+H]⁺ found: 313.1.

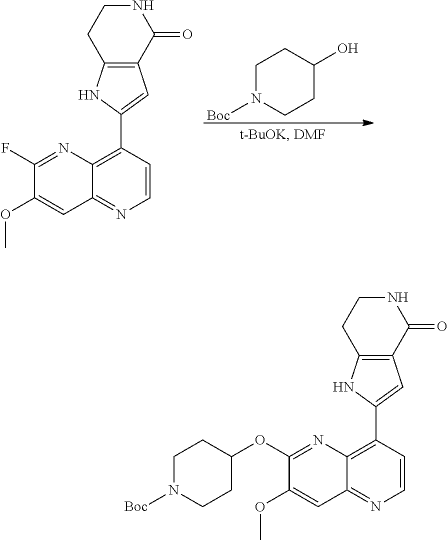 [SMILES: COc1cc2nccc(-c3cc4c([nH]3)CCNC4=O)c2nc1F ; CC(C)(C)OC(=O)N1CCC(O)CC1 ; COc1cc2nccc(-c3cc4c([nH]3)CCNC4=O)c2nc1OC1CCN(C(=O)OC(C)(C)C)CC1]

To a stirred solution of tert-butyl 4-hydroxypiperidine-1-carboxylate (193.34 mg, 0.960 mmol, 3 equiv) in t-BuOK (1M in THF, 0.35 mL, 0.352 mmol, 1.1 equiv) was added a solution of 2-(6-fluoro-7-methoxy-1,5-naphthyridin-4-yl)-1H,5H,6H,7H-pyrrolo[3,2-c]pyridin-4-one (100 mg, 0.32 mmol, 1.00 equiv) dropwiseat 0° C. under air atmosphere. The resulting mixture was stirred for overnight at room temperature under air atmosphere. The reaction was quenched with water (2 mL) at room temperature. The residue was purified by silica gel column chromatography, eluted with CH₂Cl2/MeOH (10:1) to afford tert-butyl 4-[(3-methoxy-8-(4-oxo-1H,5H,6H,7H-pyrrolo[3,2-c]pyridin-2-yl)-1,5-naphthyridin-2-yl)oxy]piperidine-1-carboxylate (130 mg, 82.26%) as a yellow solid.

LC-MS: [M+H]⁺ found: 494.15.

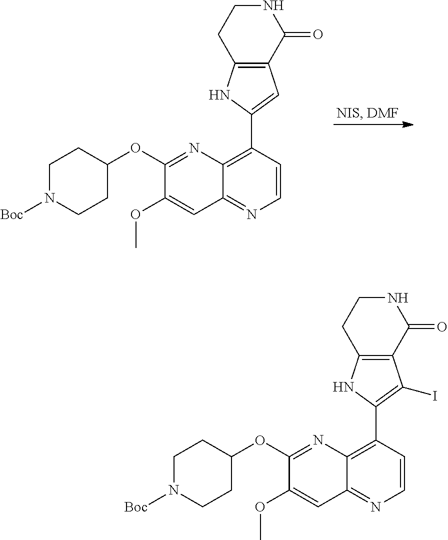 [SMILES: COc1cc2nccc(-c3cc4c([nH]3)CCNC4=O)c2nc1OC1CCN(C(=O)OC(C)(C)C)CC1 ; COc1cc2nccc(-c3[nH]c4c(c3I)C(=O)NCC4)c2nc1OC1CCN(C(=O)OC(C)(C)C)CC1]

To a stirred solution of tert-butyl 4-[(3-methoxy-8-(4-oxo-1H,5H,6H,7H-pyrrolo[3,2-c]pyridin-2-yl)-1,5-naphthyridin-2-yl)oxy]piperidine-1-carboxylate (120 mg, 0.24 mmol, 1.00 equiv) in dimethylformamide (4.00 mL) was added NIS (54 mg, 0.24 mmol, 1.00 equiv) dropwise at 0° C. under air atmosphere. The resulting mixture was stirred for overnight at room temperature under air atmosphere. The reaction was quenched by the addition of sodium hyposulfite (sat.) (2 mL) at room temperature. The resulting mixture was concentrated under reduced pressure. The residue was purified by silica gel column chromatography, eluted with CH₂Cl₂/MeOH (10:1) to afford tert-butyl 4-[(8-{3-iodo-4-oxo-1H,5H,6H,7H-pyrrolo[3,2-c]pyridin-2-yl}-3-methoxy-1,5-naphthyridin-2-yl)oxy]piperidine-1-carboxylate (130 mg, 86.32%) as a yellow solid.

LC-MS: [M+H]⁺ found: 620.05.

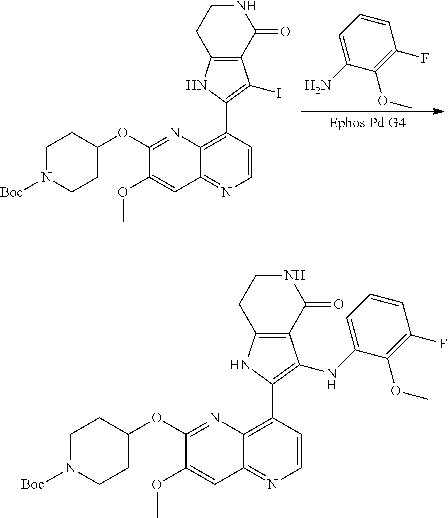

To a stirred mixture of caesio methaneperoxoate tert-butyl 4-[(8-{3-iodo-4-oxo-1H,5H,6H,7H-pyrrolo[3,2-c]pyridin-2-yl}-3-methoxy-1,5-naphthyridin-2-yl)oxy]piperidine-1-carboxylate caesium (100 mg, 0.16 mmol, 1.00 equiv), Cs₂CO₃ (115 mg, 0.35 mmol, 2.00 equiv) and 3-fluoro-2-methoxyaniline (50 mg, 0.35 mmol, 2 equiv) in dimethylformamide (2.00 mL) was added Ephos Pd G4 (16 mg, 0.018 mmol, 0.10 equiv) in portions at room temperature under argon atmosphere. The resulting mixture was stirred for 30 min at 50° C. under argon atmosphere. The resulting mixture was concentrated under reduced pressure. The residue was purified by silica gel column chromatography, eluted with CH₂Cl₂/MeOH (10:1) to afford tert-butyl 4-[(8-{3-[(3-fluoro-2-methoxyphenyl)amino]-4-oxo-1H,5H,6H,7H-pyrrolo[3,2-c]pyridin-2-yl}-3-methoxy-1,5-naphthyridin-2-yl)oxy]piperidine-1-carboxylate (110 mg, 97.91%) as a yellow solid.

LC-MS: [M+H]⁺ found: 633.15.

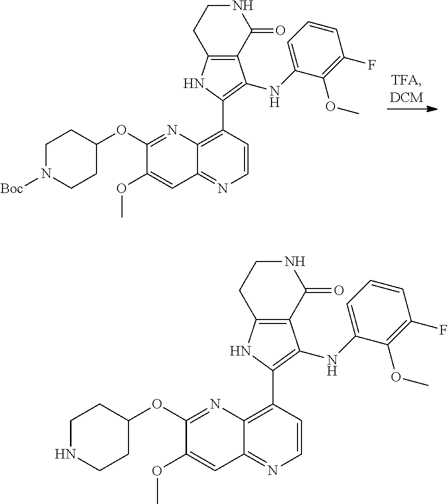 [SMILES: COc1cc2nccc(-c3[nH]c4c(c3Nc3cccc(F)c3OC)C(=O)NCC4)c2nc1OC1CCN(C(=O)OC(C)(C)C)CC1 ; COc1cc2nccc(-c3[nH]c4c(c3Nc3cccc(F)c3OC)C(=O)NCC4)c2nc1OC1CCNCC1]

To a stirred solution of tert-butyl 4-[(8-{3-[(3-fluoro-2-methoxyphenyl)amino]-4-oxo-1H,5H,6H,7H-pyrrolo[3,2-c]pyridin-2-yl}-3-methoxy-1,5-naphthyridin-2-yl)oxy]piperidine-1-carboxylate (100 mg, 0.15 mmol, 1.00 equiv) in DCM (4.00 mL) was added TFA (1.00 mL) dropwise at 0° C. under air atmosphere. The resulting mixture was stirred for 2 h at room temperature under air atmosphere. Desired product could be detected by LCMS. The resulting mixture was concentrated under reduced pressure. The crude product was used in the next step directly without further purification.

LC-MS: [M+H]⁺ found: 533.15.

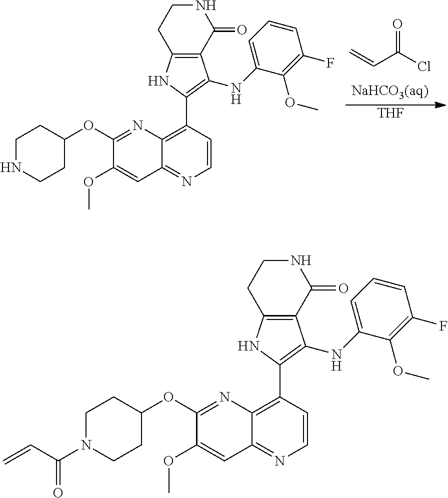

To a stirred solution of 3-[(3-fluoro-2-methoxyphenyl)amino]-2-[7-methoxy-6-(piperi din-4-yloxy)-1,5-naphthyridin-4-yl]-1H,5H,6H,7H-pyrrolo[3,2-c]pyridin-4-one (84 mg, 0.15 mmol, 1.00 equiv) in NaHCO₃ (sat.) (4.00 mL) and tetrahydrofuran (4.00 mL) was added acryloyl chloride (14.28 mg, 0.158 mmol, 1 equiv) dropwise at 0° C. under air atmosphere. The resulting mixture was stirred for 2 h at room temperature under air atmosphere. Desired product could be detected by LCMS. The resulting mixture was extracted with CH₂Cl₂ (3×10 mL), dried over anhydrous Na₂SO₄. After filtration, the filtrate was concentrated under reduced pressure. The crude product was purified by Prep-HPLC with the following conditions (Column: YMC-Actus Triart C18 ExRS, 30*150 mm, 5 μm; Mobile Phase A: Water (10 mmol/L NH4HCO3), Mobile Phase B: ACN; Flow rate: 60 mL/min; Gradient: 22% B to 46% B in 9 min, 46% B; Wave Length: 254/220 nm; RT1 (min): 9.28) to afford 3-[(3-fluoro-2-methoxyphenyl)amino]-2-(7-methoxy-6-{[1-(prop-2-enoyl)piperidin-4-yl]oxy}-1,5-naphthyridin-4-yl)-1H,5H,6H,7H-pyrrolo[3,2-c]pyridin-4-one (19.1 mg, 20.06%) as a yellow solid.

LC-MS: [M+H]⁺ found: 586.95.

¹H NMR (300 MHz, DMSO-d₆) δ 12.31 (s, 1H), 8.41 (d, 1H), 7.74 (d, 2H), 7.46 (d, 1H), 6.69-6.48 (m, 3H), 6.31-6.37 (m, 1H), 6.03-6.07 (m, 1H), 5.73-5.77 (m, 1H), 5.50-5.44 (m, 1H), 5.29 (s, 1H), 4.14 (d, 3H), 3.88-4.02 (m, 6H), 3.64-3.74 (m, 3H), 3.00 (t, 2H), 2.40-2.20 (d, 4H).

### Example 267. 3-[(3-fluoro-2-methoxyphenyl)amino]-2-{furo[3,2-b]pyridin-7-yl}-1H,5H,6H,7H-pyrrolo[3,2-c]pyridin-4-one (compound 553)

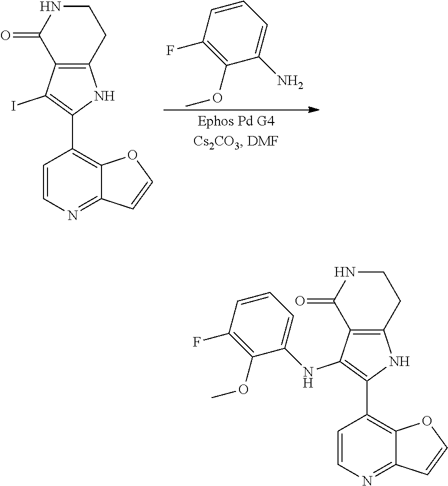

To a stirred mixture of 2-{furo[3,2-b]pyridin-7-yl}-3-iodo-1H,5H,6H,7H-pyrrolo[3,2-c]pyridin-4-one (80 mg, 0.211 mmol, 1.00 equiv) and caesio methaneperoxoate caesium (137.92 mg, 0.422 mmol, 2 equiv) in dimethylformamide (2 mL) were added Ephos Pd G4 (19.38 mg, 0.021 mmol, 0.1 equiv) and 3-fluoro-2-methoxyaniline (26.80 mg, 0.190 mmol, 0.9 equiv) in portions at room temperature under argon atmosphere. The resulting mixture was stirred for 1 h at 50 degrees C. under argon atmosphere. The resulting mixture was concentrated under reduced pressure. The residue was purified by silica gel column chromatography, eluted with DCM/MeOH (10:1) to afford crude product (62 mg). The crude product (62 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 20% B to 50% B in 7 min, 50% B: Wave Length: 254/220 nm; RT1 (min): 6.12) to afford 3-[(3-fluoro-2-methoxyphenyl)amino]-2-{furo[3,2-b]pyridin-7-yl}-1H,5H,6H,7H-pyrrolo[3,2-c]pyridin-4-one (42.8 mg, 51.02%) as a white solid.

LC-MS: [M+H]⁺ found: 393.25.

¹H NMR (400 MHz, DMSO-d6) δ 11.58 (s, 1H), 8.33-8.29 (m, 2H), 7.54 (s, 1H), 7.23-7.11 (m, 3H), 6.59-6.43 (m, 2H), 5.99 (d, 1H), 3.92 (s, 3H), 3.42-3.45 (m, 2H), 2.93-2.89 (m, 2H).

### Example 268. 3-[(3-chloro-2-methoxyphenyl)amino]-2-(7-methoxy-6-{[1-(prop-2-enoyl)piperidin-4-yl]oxy}-1,5-naphthyridin-4-yl)-1H,5H,6H,7H-pyrrolo[3,2-c]pyridin-4-one (compound 731)

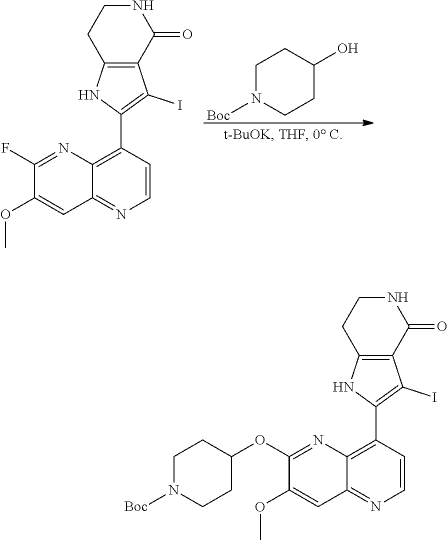 [SMILES: COc1cc2nccc(-c3[nH]c4c(c3I)C(=O)NCC4)c2nc1F ; CC(C)(C)OC(=O)N1CCC(O)CC1 ; COc1cc2nccc(-c3[nH]c4c(c3I)C(=O)NCC4)c2nc1OC1CCN(C(=O)OC(C)(C)C)CC1]

To a stirred solution of tert-butyl 4-hydroxypiperidine-1-carboxylate (193 mg, 0.96 mmol, 3.00 equiv) in THF (2.00 mL) was added 2-(6-fluoro-7-methoxy-1,5-naphthyridin-4-yl)-1H,5H,6H,7H-pyrrolo[3,2-c]pyridin-4-one (100 mg, 0.32 mmol, 1.00 equiv) and t-BuOK (0M in THF, 0.35 mL, 0.35 mmol, 1.10 equiv) dropwise at 0 C. The resulting mixture was stirred for overnight at room temperature under air atmosphere. The reaction was quenched with water at room temperature. The residue was purified by silica gel column chromatography, eluted with CH₂Cl2/MeOH (10:1) to afford tert-butyl 4-[(3-methoxy-8-{4-oxo-1H,5H,6H,7H-pyrrolo[3,2-c]pyridin-2-yl}-1,5-naphthyridin-2-yl)oxy]piperidine-1-carboxylate (130 mg, 82.26%) as a yellow solid.

LC-MS: [M−H]⁻ found: 494.15.

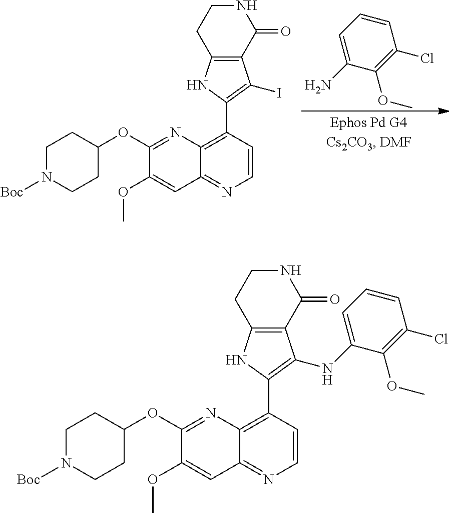

To a stirred mixture of tert-butyl 4-[(8-{3-iodo-4-oxo-1H,5H,6H,7H-pyrrolo[3,2-c]pyridin-2-yl}-3-methoxy-1,5-naphthyridin-2-yl)oxy]piperidine-1-carboxylate (120 mg, 0.19 mmol, 1.00 equiv) and 3-chloro-2-methoxyaniline (30 mg, 0.19 mmol, 1.00 equiv) in dimethylformamide (3.00 mL) were added Ephos Pd G4 (17 mg, 0.02 mmol, 0.10 equiv) and Cs₂CO₃ (126 mg, 0.38 mmol, 2.00 equiv) in portions at room temperature under argon atmosphere. The resulting mixture was stirred for 30 min at 50° C. under argon atmosphere. The resulting mixture was concentrated under reduced pressure. The residue was purified by silica gel column chromatography, eluted with CH₂Cl₂/MeOH (10:1) to afford tert-butyl 4-[(8-{3-[(3-chloro-2-methoxyphenyl)amino]-4-oxo-1H,5H,6H,7H-pyrrolo[3,2-c]pyridin-2-yl}-3-methoxy-1,5-naphthyridin-2-yl)oxy]piperidine-1-carboxylate (120 mg, 95.43%) as a yellow solid.

LC-MS: [M−H]⁻ found: 649.30.

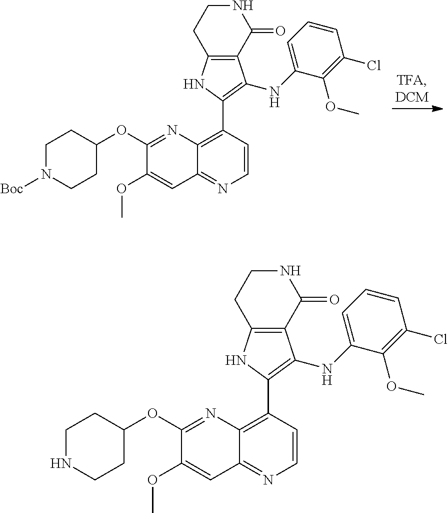 [SMILES: COc1cc2nccc(-c3[nH]c4c(c3Nc3cccc(Cl)c3OC)C(=O)NCC4)c2nc1OC1CCN(C(=O)OC(C)(C)C)CC1 ; COc1cc2nccc(-c3[nH]c4c(c3Nc3cccc(Cl)c3OC)C(=O)NCC4)c2nc1OC1CCNCC1]

To a stirred solution of tert-butyl 4-[(8-{3-[(3-chloro-2-methoxyphenyl)amino]-4-oxo-1H,5H,6H,7H-pyrrolo[3,2-c]pyridin-2-yl}-3-methoxy-1,5-naphthyridin-2-yl)oxy]piperidine-1-carboxylate (100 mg, 0.15 mmol, 1.00 equiv) in DCM (0.8 mL) was added TFA (0.20 mL) dropwise at 0° C. The resulting mixture was stirred for 1 h at room temperature under air atmosphere. Desired product could be detected by LCMS. The resulting mixture was concentrated under reduced pressure. The crude product was used in the next step directly without further purification

LC-MS: [M−H]⁻ found: 549.10.

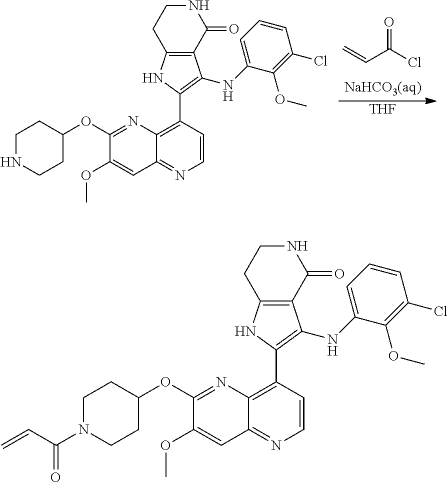

To a stirred solution of 3-[(3-chloro-2-methoxyphenyl)amino]-2-[7-methoxy-6-(piperidin-4-yloxy)-1,5-naphthyridin-4-yl]-1H,5H,6H,7H-pyrrolo[3,2-c]pyridin-4-one (84 mg, 0.15 mmol, 1.00 equiv) in NaHCO₃ (sat.) (4.00 mL) and THF (4.00 mL) was added acryloyl chloride (14 mg, 0.15 mmol, 1.00 equiv) dropwise at 0° C. The resulting mixture was stirred for 2 h at room temperature under air atmosphere. The resulting mixture was extracted with CH₂Cl₂ (3×20 mL), dried over anhydrous Na₂SO₄. After filtration, the filtrate was concentrated under reduced pressure. The crude product was purified by Prep-HPLC with the following conditions (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 27% B to 46% B in 10 min, 46% B; Wave Length: 254/220 nm; RT1 (min): 9.67) to afford 3-[(3-chloro-2-methoxyphenyl)amino]-2-(7-methoxy-6-{[1-(prop-2-enoyl)piperidin-4-yl]oxy}-1,5-naphthyridin-4-yl)-1H,5H,6H,7H-pyrrolo[3,2-c]pyridin-4-one (23.2 mg, 24.81%) as a yellow solid.

LC-MS: [M+H]⁺ found: 603.25.

¹H NMR (300 MHz, DMSO-d6) δ 12.32 (s, 1H), 8.43 (d, 1H), 7.77 (s, 1H), 7.67 (s, 1H), 7.41 (d, 1H), 6.91-6.75 (m, 1H), 6.69-6.59 (m, 2H), 6.37-6.31 (m, 1H), 6.20-6.17 (m, 1H), 5.77-5.73 (m, 1H), 5.45 (t, 1H), 5.28 (s, 1H), 4.12 (s, 3H), 4.03 (s, 3H), 3.99-3.80 (m, 3H), 3.75-3.62 (m, 3H), 2.99 (t, 2H), 2.30-2.10 (m, 4H).

### Example 269. 2-(2-{4-[(2E)-4-(dimethylamino)but-2-enoyl]piperazin-1-yl}pyrido[3,2-d]pyrimidin-8-yl)-3-[(3-fluoro-2-methoxyphenyl)amino]-1H,5H,6H,7H-pyrrolo[3,2-c]pyridin-4-one (compound 577)

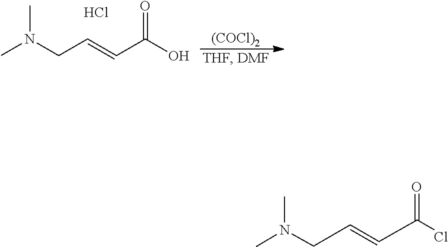

To a stirred solution of (2E)-4-(dimethylamino)but-2-enoic acid hydrochloride (100 mg, 0.60 mmol, 1.00 equiv) in THF (3 mL) and DMF 1 drop was added (COCl)2 (84 mg, 0.66 mmol, 1.10 equiv) dropwise at 0° C. The resulting mixture was stirred for 2 h at 0° C. TLC (DCM:MeOH=5:1) showed a new spot was detected. The resulting mixture was used in the next step directly without further purification.

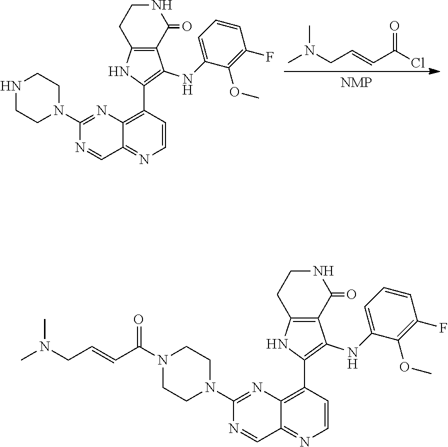

To a stirred solution of 3-[(3-fluoro-2-methoxyphenyl)amino]-2-[2-(piperazin-1-yl)pyrido[3,2-d]pyrimidin-8-yl]-1H,5H,6H,7H-pyrrolo[3,2-c]pyridin-4-one (100 mg, 0.21 mmol, 1.00 equiv) in NMP (3 mL) was added the solution of (2E)-4-(dimethylamino)but-2-enoyl chloride (30 mg, 0.21 mmol, 1.00 equiv) in 1 mL THF dropwise at 0° C. The resulting mixture was stirred for 2 h at 0° C. The crude product was purified by Prep-HPLC with the following conditions (Column: XBridge Prep C18 OBD Column, 30*100 mm, 5 μm; Mobile Phase A: Water (10 mmol/L NH4HCO3+0.1% NH3·H2O), Mobile Phase B: ACN; Flow rate: 60 mL/min; Gradient: 27% B to 40% B in 10 min, 40% B; Wave Length: 254/220 nm; RT1 (min): 9.67) to afford 2-(2-{4-[(2E)-4-(dimethylamino)but-2-enoyl]piperazin-1-yl}pyrido[3,2-d]pyrimidin-8-yl)-3-[(3-fluoro-2-methoxyphenyl)amino]-1H,5H,6H,7H-pyrrolo[3,2-c]pyridin-4-one (19.0 mg, 14.78%) as a yellow solid.

LC-MS: (M+H)⁺ found: 600.05.

¹H NMR (300 MHz, Chloroform-d) δ 12.71 (s, 1H), 9.26 (s, 1H), 8.43 (d, J=4.9 Hz, 1H), 7.75 (s, 1H), 7.45 (d, J=4.9 Hz, 1H), 6.02-6.90 (m, 1H), 6.73-6.40 (m, 3H), 6.08-6.01 (m, 1H), 5.30 (d, J=2.6 Hz, 1H), 4.29-3.73 (m, 11H), 3.73-3.63 (m, 2H), 3.19 (d, J=5.8 Hz, 2H), 3.02 (t, J=6.7 Hz, 2H), 2.35 (s, 6H).

### Example 270. 3-[(3-fluoro-2-methoxyphenyl)amino]-2-{2-[4-(prop-2-enoyl)piperazin-1-yl]pyrido[3,2-d]pyrimidin-8-yl}-1H,5H,6H,7H-pyrrolo[3,2-c]pyridin-4-one (compound 576)

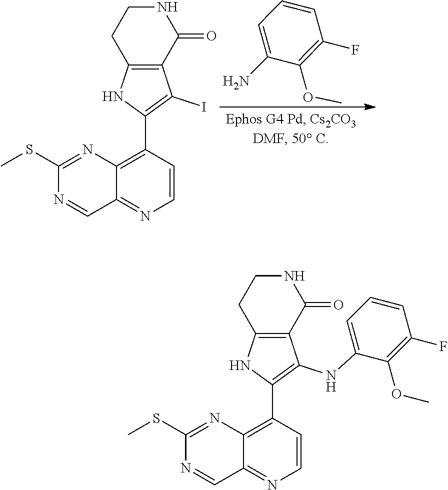

To a solution of 3-iodo-2-[2-(methylsulfanyl)pyrido[3,2-d]pyrimidin-8-yl]-1H,5H,6H,7H-pyrrolo[3,2-c]pyridin-4-one (330 mg, 0.75 mmol, 1.00 equiv) and 3-fluoro-2-methoxyaniline (117 mg, 0.83 mmol, 1.10 equiv) in DMF was added Cs₂CO₃ (738 mg, 2.26 mmol, 3.00 equiv), EPhos Pd G4 (69 mg, 0.08 mmol, 0.10 equiv). The resulting mixture was stirred for 1 h at 50° C. under argon atmosphere. The resulting mixture was concentrated under reduced pressure. The residue was purified by silica gel column chromatography, eluted with CH₂Cl₂/MeOH (10:1) to afford 2-bromo-3-[(3-chloro-2-methoxyphenyl)amino]-5H,6H,7H-pyrazolo[1,5-a]pyrazin-4-one (226 mg, 66.47%) as a yellow solid.

LC-MS: (M+H)⁺ found: 451.

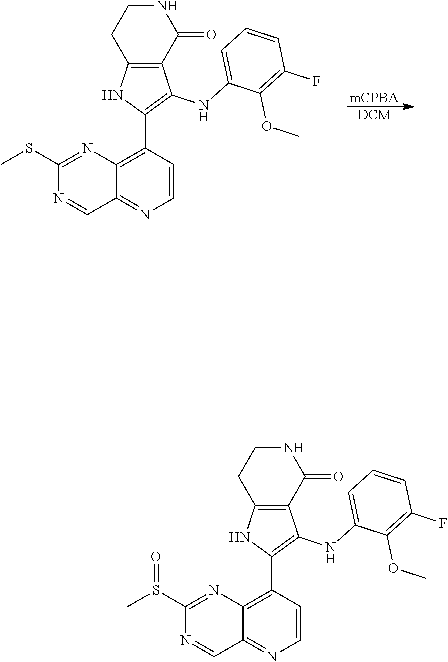 [SMILES: COc1c(F)cccc1Nc1c(-c2ccnc3cnc(SC)nc23)[nH]c2c1C(=O)NCC2 ; COc1c(F)cccc1Nc1c(-c2ccnc3cnc(S(C)=O)nc23)[nH]c2c1C(=O)NCC2]

To a sealed tube were added 3-[(3-fluoro-2-methoxyphenyl)amino]-2-[2-(methylsulfanyl)pyrido[3,2-d]pyrimidin-8-yl]-1H,5H,6H,7H-pyrrolo[3,2-c]pyridin-4-one (206 mg, 0.46 mmol, 1.00 equiv) and DCM (15 mL), at 0° C., m-CPBA (87 mg, 0.50 mmol, 1.10 equiv) in DCM was added and stirred for 1 h at room temperature. The reaction was quenched by the addition of Water (20 mL) at 25° C. The resulting mixture was extracted with CH₂Cl₂ (2×50 mL), dried over anhydrous Na₂SO₄. Afler filtration, the filtrate was concentrated under reduced pressure to give 3-[(3-fluoro-2-methoxyphenyl)amino]-2-{2-methanesulfinylpyrido[3,2-d]pyrimidin-8-yl}-1H,5H,6H,7H-pyrrolo[3,2-c]pyridin-4-one (200 mg). The crude product was used in the next step directly without further purification.

LC-MS: (M+H)⁺ found: 467.

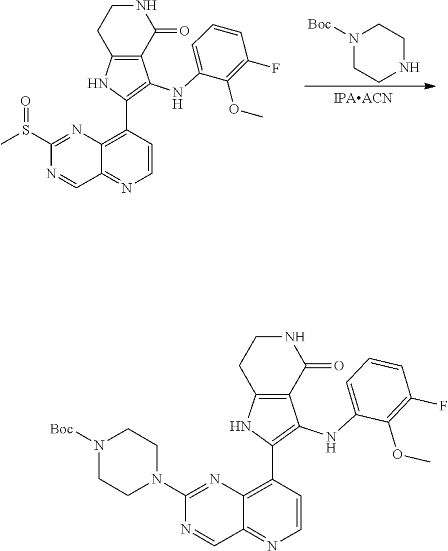

To a sealed tube were added 3-[(3-fluoro-2-methoxyphenyl)amino]-2-{2-methanesulfinylpyrido[3,2-d]pyrimidin-8-yl}-1H,5H,6H,7H-pyrrolo[3,2-c]pyridin-4-one (206 mg, 0.44 mmol, 1.00 equiv), tert-butyl piperazine-1-carboxylate (411 mg, 2.21 mmol, 5.00 equiv) in IPA (5 mL) and ACN (5 mL) stirred overnight at 70° C. under N2 atmosphere. The resulting mixture was concentrated under vacuum to give tert-butyl 4-(8-{3-[(3-fluoro-2-methoxyphenyl)amino]-4-oxo-1H,5H,6H,7H-pyrrolo[3,2-c]pyridin-2-yl}pyrido[3,2-d]pyrimidin-2-yl)piperazine-1-carboxylate (400 mg, crude) as brown yellow solid.

LC-MS: M+H)⁺ found: 589.

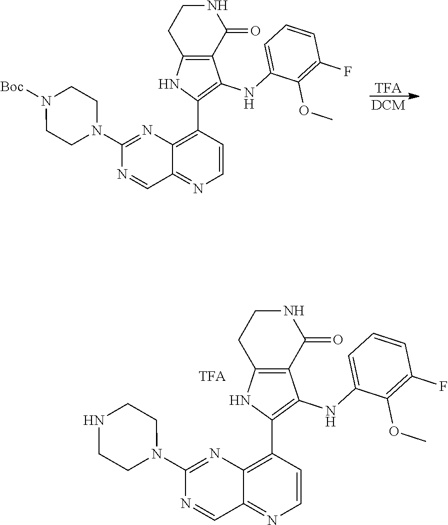 [SMILES: COc1c(F)cccc1Nc1c(-c2ccnc3cnc(N4CCN(C(=O)OC(C)(C)C)CC4)nc23)[nH]c2c1C(=O)NCC2 ; COc1c(F)cccc1Nc1c(-c2ccnc3cnc(N4CCNCC4)nc23)[nH]c2c1C(=O)NCC2]

To a stirred solution of tert-butyl 4-(8-{3-[(3-fluoro-2-methoxyphenyl)amino]-4-oxo-1H,5H,6H,7H-pyrrolo[3,2-c]pyridin-2-yl}pyrido[3,2-d]pyrimidin-2-yl)piperazine-1-carboxylate (400 mg, crude) in DCM (10 mL) was added TFA (3 mL) at room temperature under nitrogen atmosphere. The resulting mixture was stirred for 2 h at room temperature under hydrogen atmosphere. Desired product could be detected by LCMS. The resulting mixture was concentrated under reduced pressure. The residue was purified by reverse flash chromatography (mobile phase, MeCN in water, 0% to 100%) to afford 3-[(3-fluoro-2-methoxyphenyl)amino]-2-[2-(piperazin-1-yl)pyrido[3,2-d]pyrimidin-8-yl]-1H,5H,6H,7H-pyrrolo[3,2-c]pyridin-4-one; trifluoroacetic acid (300 mg, 73.27%) as brown solid.

LC-MS: (M+H)⁺ found: 489.

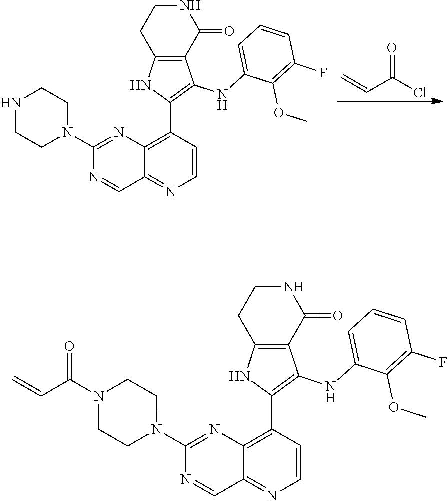

To a stirred solution of 3-[(3-fluoro-2-methoxyphenyl)amino]-2-[2-(piperazin-1-yl)pyrido[3,2-d]pyrimidin-8-yl]-1H,5H,6H,7H-pyrrolo[3,2-c]pyridin-4-one (100 mg, 0.21 mmol, 1.00 equiv) in THF (3 mL) was added NaHCO₃ (0.1 mL) at 0° C. To the above mixture was added acryloyl chloride (17 mg, 0.184 mmol, 0.90 equiv) dropwise at 0° C. The resulting mixture was stirred for 2 h at 0° C. Desired product could be detected by LCMS. The crude product was purified by Prep-HPLC with the following conditions (Column: XBridge Prep C18 OBD Column, 30*100 mm, 5 μm; Mobile Phase A: Water (10 mmol/L NH₄HCO₃+0.1% NH3·H2O), Mobile Phase B. ACN; Flow rate: 60 mL/min; Gradient: 27% B to 40% B in 10 min, 40% B; Wave Length: 254/220 nm; RT1 (min): 9.67) to afford 3-[(3-fluoro-2-methoxyphenyl)amino]-2-{2-[4-(prop-2-enoyl)piperazin-1-yl]pyrido[3,2-d]pyrimidin-8-yl}-1H,5H,6H,7H-pyrrolo[3,2-c]pyridin-4-one (13.8 mg, 11.82%) as a yellow solid.

LC-MS: (M+H)⁺ found: 543.05.

¹H NMR (300 MHz, Chloroform-d) δ 12.63 (s, 1H), 9.19 (s, 1H), 8.36 (d, J=4.9 Hz, 1H), 7.69 (s, 1H), 7.38 (d, J=4.9 Hz, 1H), 6.68-6.40 (m, 3H), 6.38-6.30 (m, 1H), 6.02-5.92 (m, 1H), 5.80-5.68 (m, 1H), 5.26 (s, 1H), 4.22-3.67 (m, 11H), 3.61 (m, 2H), 2.93 (t, J=6.7 Hz, 2H).

### Example 271. 3-[(3-chloro-2-methoxyphenyl)amino]-2-{2-[(1-ethyl-1,2,3-triazol-4-yl)amino]pyrimidin-4-yl}-1H,5H,6H,7H-pyrrolo[3,2-c]pyridin-4-one (compound 594)

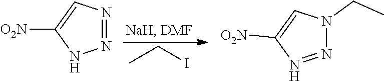

To a stirred solution of 4-nitro-3H-1,2,3-triazole (1.0 g, 8.77 mmol, 1.00 equiv) in DMF (10 mL) was added NaH (0.37 g, 9.21 mmol, 1.05 equiv, 60%) in portions at 0 degrees C. under argon atmosphere. The resulting mixture was stirred for 30 min at 0 degrees C. under argon atmosphere. To the above mixture was added ethyl iodide (1.78 g, 11.40 mmol, 1.30 equiv) in portions at 0 degrees C. The resulting mixture was stirred for additional 1 h at room temperature. The reaction was quenched by the addition of Water (10 mL) at 0 degrees C. The resulting mixture was extracted with EtOAc (3×100 mL). The combined organic layers were washed with water (3×100 mL), dried over anhydrous Na₂SO₄. After filtration, the filtrate was concentrated under reduced pressure. The residue was purified by silica gel column chromatography, eluted with PE/EtOAc (1:1) to afford 1-ethyl-4-nitro-1,2,3-triazole (700 mg, 56.18%) as a yellow oil.

¹H NMR (400 MHz, DMSO-d6) δ 8.71 (s, 1H), 4.59 (q, J=7.3 Hz, 2H), 1.51 (t, J=7.3 Hz, 3H).

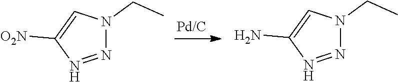

To a stirred solution of 1-ethyl-4-nitro-1,2,3-triazole (120 mg, 0.84 mmol, 1.00 equiv) in EtOH (6 mL) was added Pd/C (25 mg, 21% w/w) in portions at room temperature under argon atmosphere. The resulting mixture was stirred for 2 h at room temperature under hydrogen atmosphere. The resulting mixture was filtered, the filter cake was washed with MeOH (3×30 mL). The filtrate was concentrated under reduced pressure. The residue was purified by silica gel column chromatography, eluted with PE/EA (2:1) to afford 1-ethyl-1,2,3-triazol-4-amine (90 mg, 95.05%) as a yellow solid.

LC-MS: (M+H)⁺ found: 113.4.

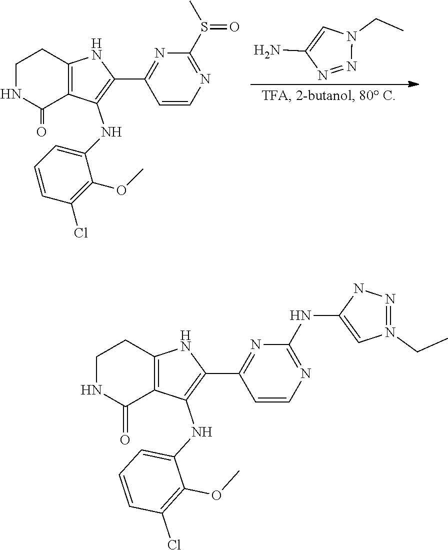

To a stirred solution of 3-[(3-chloro-2-methoxyphenyl)amino]-2-(2-methanesulfinylpyrimidin-4-yl)-1H,5H,6H,7H-pyrrolo[3,2-c]pyridin-4-one (80 mg, 0.19 mmol, 1.00 equiv) in 2-butanol (2 mL) was added 1-ethyl-1,2,3-triazol-4-amine (208 mg, 1.85 mmol, 10.00 equiv) and TFA (42 mg, 0.37 mmol, 2.00 equiv) in portions at room temperature under argon atmosphere. The resulting mixture was stirred for 36 h at 80 degrees C. under argon atmosphere. The resulting mixture was concentrated under reduced pressure. The crude product (400 mg) was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 32% B to 55% B in 7 min, 55% B; Wave Length: 254/220 nm; RT1 (min): 6.62) to afford 3-[(3-chloro-2-methoxyphenyl)amino]-2-{2-[(1-ethyl-1,2,3-triazol-4-yl)amino]pyrimidin-4-yl}-1H,5H,6H,7H-pyrrolo[3,2-c]pyridin-4-one (21.2 mg, 23.13%) as a white solid.

LC-MS: (M+H)⁺ found: 479.95.

¹H NMR (400 MHz, DMSO-d6) δ 11.45 (s, 1H), 9.80 (s, 1H), 8.25 (d, J=5.3 Hz, 1H), 8.13 (s, 1H), 7.78 (s, 1H), 7.20 (t, J=2.6 Hz, 1H), 6.83-6.72 (m, 2H), 6.70 (d, J=5.3 Hz, 1H), 6.36-6.27 (m, 1H), 4.38-4.28 (m, 2H), 3.90 (s, 3H), 3.46-3.37 (m, 2H), 2.91 (t, J=6.8 Hz, 2H), 1.43 (t, J=7.3 Hz, 3H).

### Example 272. 3-[(3-chloro-2-methoxyphenyl)amino]-2-(2-[[(1-methylpyrazol-3-yl)methyl]amino]pyrimidin-4-yl)-1H,5H,6H,7H-pyrrolo[3,2-c]pyridin-4-one (compound 709)

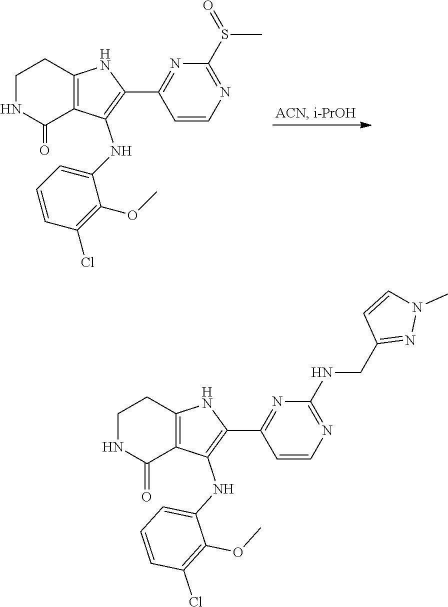 [SMILES: COc1c(Cl)cccc1Nc1c(-c2ccnc(S(C)=O)n2)[nH]c2c1C(=O)NCC2 ; COc1c(Cl)cccc1Nc1c(-c2ccnc(NCc3ccn(C)n3)n2)[nH]c2c1C(=O)NCC2]

A solution of 3-[(3-chloro-2-methoxyphenyl) amino]-2-(2-methanesulfinylpyrimidin-4-yl)-1H,5H,6H,7H-pyrrolo[3,2-c] pyridin-4-one (80 mg, 0.19 mmol, 1.00 equiv) and (1-methyl-1H-pyrazol-3-yl) methanamine (206 mg, 1.85 mmol, 10.00 equiv) in ACN (1.00 mL) and i-PrOH (1.00 mL) was stirred for overnight at 80 degrees C. under nitrogen atmosphere. The resulting mixture was concentrated under reduced pressure. The crude product (100 mg) was purified by Prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 22% B to 52% B in 7 min, 52% B; Wave Length: 254 nm; RT1 (min): 6.5) to afford 3-[(3-chloro-2-methoxyphenyl)amino]-2-(2-[[(1-methylpyrazol-3-yl)methyl]amino]pyrimidin-4-yl)-1H,5H,6H,7H-pyrrolo[3,2-c]pyridin-4-one (4.2 mg, 4.71%) as a light yellow solid.

LC-MS: M+H found: 479.30.

¹H NMR (300 MHz, DMSO-d6) δ 11.62 (s, 1H), 8.08 (d, J=5.3 Hz, 1H), 7.86 (s, 1H), 7.56 (d, J=2.2 Hz, 1H), 7.14 (s, 1H), 6.83-6.72 (m, 3H), 6.47 (d, J=5.3 Hz, 1H), 6.38-6.36 (m, 1H), 6.12 (d, J=2.2 Hz, 1H), 4.52 (d, J=5.9 Hz, 2H), 3.87 (s, 3H), 3.79 (s, 3H), 3.46-3.36 (m, 2H), 2.89-2.87 (m, 2H).

### Example 273. 3-[(3-chloro-2-methoxyphenyl) amino]-2-(2-[[(3,3-difluorocyclobutyl) methyl] amino] pyrimidin-4-yl)-1H,5H,6H,7H-pyrrolo[3,2-c] pyridin-4-one (compound

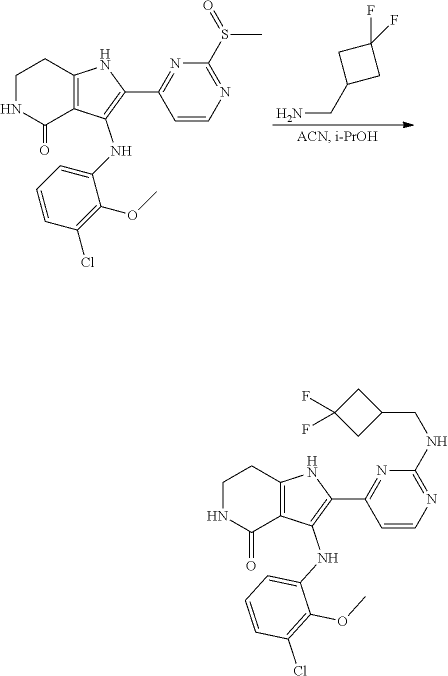 [SMILES: COc1c(Cl)cccc1Nc1c(-c2ccnc(S(C)=O)n2)[nH]c2c1C(=O)NCC2 ; NCC1CC(F)(F)C1 ; COc1c(Cl)cccc1Nc1c(-c2ccnc(NCC3CC(F)(F)C3)n2)[nH]c2c1C(=O)NCC2]

Into a 10-mL sealed tube purged and maintained with an inert atmosphere of argon, was placed 3-[(3-chloro-2-methoxyphenyl) amino]-2-(2-methanesulfinylpyrimidin-4-yl)-1H,5H,6H,7H-pyrrolo[3,2-c] pyridin-4-one (80.00 mg, 0.19 mmol, 1.00 equiv), ACN (1.00 mL, 0.024 mmol, 0.13 equiv), i-PrOH (1.00 mL), 1-(3,3-difluorocyclobutyl) methanamine (224 mg, 1.85 mmol, 10.00 equiv). The resulting solution was stirred for overnight at 110 degrees C. The resulting mixture was concentrated under reduced pressure. The crude product (50 mg) was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 19% B to 37% B in 8 min, 37% B; Wave Length: 254/220 nm; RT1 (min): 7.68) to afford 3-[(3-chloro-2-methoxyphenyl) amino]-2-(2-[[(3,3-difluorocyclobutyl) methyl] amino] pyrimidin-4-yl)-1H,5H,6H,7H-pyrrolo[3,2-c] pyridin-4-one (11.9 mg, 13.13%) as a light yellow solid.

LC-MS: M+H found: 489.30.

¹H NMR (400 MHz, DMSO-d6) δ 11.58 (s, 1H), 8.08 (d, J=5.3 Hz, 1H), 7.97 (s, 1H), 7.12 (t, J=2.7 Hz, 1H), 6.80-6.74 (m, 2H), 6.54 (d, J=5.3 Hz, 1H), 6.42-6.40 (m, 1H), 3.87 (s, 3H), 3.42-3.39 (m, 4H), 2.88 (t, J=6.8 Hz, 2H), 2.58-2.52 (m, 2H), 2.38-2.26 (m, 3H).

### Example 274. 3-[(3-chloro-2-methoxyphenyl) amino]-2-[2-[(3,3-difluorocyclobutyl) amino] pyrimidin-4-yl]-1H,5H,6H,7H-pyrrolo[3,2-c] pyridin-4-one (compound 712)

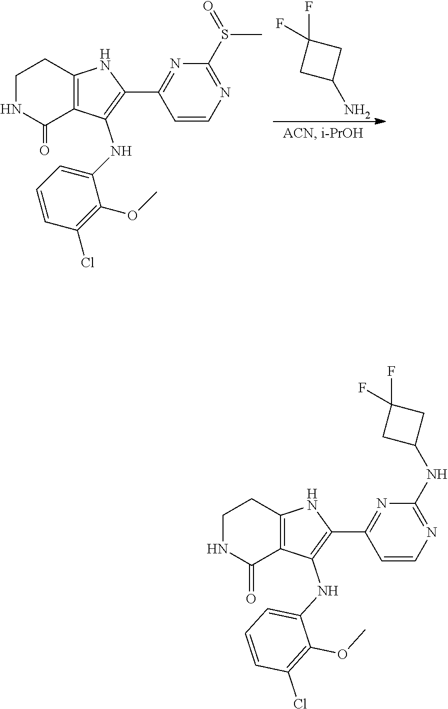

Into a 10-mL sealed tube purged and maintained with an inert atmosphere of argon, was placed 3-[(3-chloro-2-methoxyphenyl) amino]-2-(2-methanesulfinylpyrimidin-4-yl)-1H,5H,6H,7H-pyrrolo[3,2-c] pyridin-4-one (80.00 mg, 0.19 mmol, 1.00 equiv), ACN (1.00 mL), i-PrOH (1.00 mL), 3,3-difluorocyclobutan-1-amine (198 mg, 1.85 mmol, 10.00 equiv). The resulting solution was stirred for overnight at 110 degrees C. The resulting mixture was concentrated under reduced pressure. The crude product (75 mg) was purified by Prep-HPLC with the following conditions (Column: Xselect CSH OBD Column 30*150 mm 5 um, n; Mobile Phase A: Water (0.1% FA), Mobile Phase B: ACN; Flow rate: 60 mL/min; Gradient: 22% B to 38% B in 8 min, 38% B; Wave Length: 254/220 nm; RT1 (min): 7.55) to afford 3-[(3-chloro-2-methoxyphenyl) amino]-2-[2-[(3,3-difluorocyclobutyl) amino] pyrimidin-4-yl]-1H,5H,6H,7H-pyrrolo[3,2-c] pyridin-4-one (4.6 mg, 5.18%) as a light yellow solid.

LC-MS: M+H found: 475.30.

¹H NMR (400 MHz, DMSO-d6) δ 11.55 (s, 1H), 8.09 (d, J=5.3 Hz, 1H), 7.78 (s, 1H), 7.39 (s, 1H), 7.16 (t, J=2.7 Hz, 1H), 6.86-6.78 (m, 2H), 6.52 (d, J=5.3 Hz, 1H), 6.34-6.32 (m, 1H), 4.41 (brs, 1H), 3.89 (s, 3H), 3.42-3.39 (m, 2H), 3.01-2.92 (m, 4H), 2.93-2.88 (m, 2H).

### Example 275. (7S)-3-[(3-fluoro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (compound 422c)

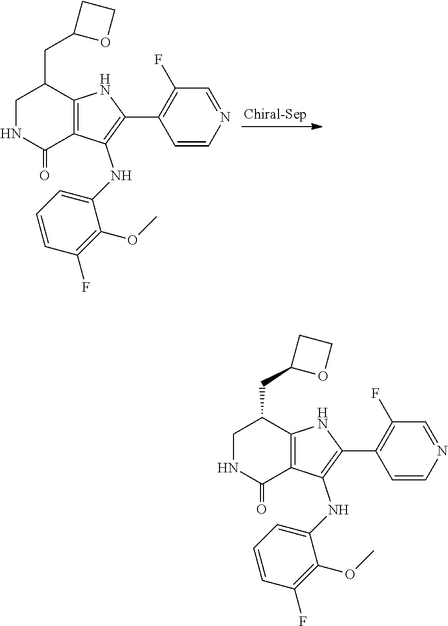 [SMILES: COc1c(F)cccc1Nc1c(-c2ccncc2F)[nH]c2c1C(=O)NCC2CC1CCO1 ; COc1c(F)cccc1Nc1c(-c2ccncc2F)[nH]c2c1C(=O)NC[C@@H]2C[C@H]1CCO1]

3-[(3-fluoro-2-methoxyphenyl)amino]-2-(3-fluoropyridin-4-yl)-7-(oxetan-2-ylmethyl)-1H,5H,6H,7H-pyrrolo[3,2-c]pyridin-4-one (300 mg) was separated by Prep-Chiral-HPLC with the following conditions (Column: CHIRALPAK IA-3, 4.6*50 mm, 3 um; Mobile Phase A: MtBE (0.1% DEA): EtOH=70: 30; Flow rate: 1 mL/min; Gradient: 0% B to 0% B; Injection Volume: 5 ul mL). This resulted in (7S)-3-[(3-fluoro-2-methoxyphenyl)amino]-2-(3-fluoropyridin-4-yl)-7-[(2S)-oxetan-2-ylmethyl]-1H,5H,6H,7H-pyrrolo[3,2-c]pyridin-4-one (4.9 mg) as a yellow solid.

LC-MS: (M+H)⁺ found: 441.

1H NMR (300 MHz, Chloroform-d) δ 11.01 (s, 1H), 8.45 (d, J=4.3 Hz, 1H), 8.15 (d, J=5.7 Hz, 1H), 7.73 (s, 1H), 7.43 (t, J=6.5 Hz, 1H), 6.72-6.50 (m, 2H), 6.10 (d, J=8.0 Hz, 1H), 5.30 (d, J=11.1 Hz, 2H), 4.89 (q, J=7.4 Hz, 1H), 4.68-4.54 (m, 1H), 4.14 (d, J=1.4 Hz, 3H), 3.76-3.37 (m, 3H), 2.73 (m, 2H), 2.23 (t, J=11.2 Hz, 1H), 1.83 (d, J=15.1 Hz, 1H).

### Example 276. 2-(7-fluoro-1,5-naphthyridin-4-yl)-3-[(3-fluoro-2-methoxyphenyl)amino]-1H,5H,6H,7H-pyrrolo[3,2-c]pyridin-4-one (Compound 826)

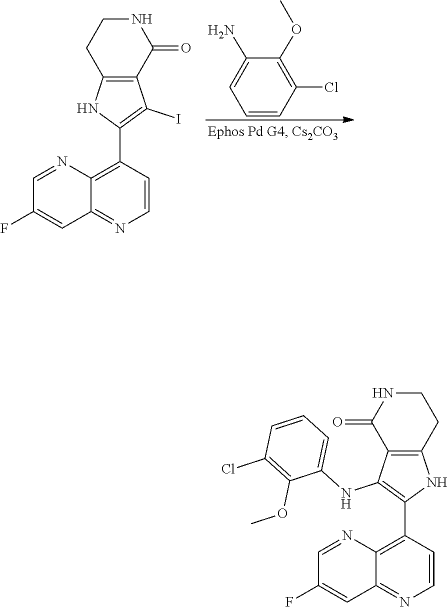

To a stirred solution of 2-(7-fluoro-1,5-naphthyridin-4-yl)-3-iodo-1H,5H,6H,7H-pyrrolo[3,2-c]pyridin-4-one (100 mg, 0.25 mmol, 1.00 equiv) and 3-chloro-2-methoxyaniline (35 mg, 0.22 mmol, 0.90 equiv) in DMF (1.00 mL) was added Ephos Pd G4 (22 mg, 0.02 mmol, 0.10 equiv) dropwise at room temperature under Ar atmosphere. The resulting mixture was stirred for 1 h at 50 degrees C. under Ar atmosphere. The reaction was monitored by LCMS. The crude product was purified by Prep-HPLC with the following conditions (Column: Xselect CSH OBD Column 30*150 mm Sum, n; Mobile Phase A: Water (0.1% FA), Mobile Phase B: ACN; Flow rate: 60 mL/min; Gradient: 30% B to 46% B in 8 min, 46% B; Wave Length: 254/220 nm; RT1 (min): 7.8) to afford 2-(7-fluoro-1,5-naphthyridin-4-yl)-3-[(3-fluoro-2-methoxyphenyl)amino]-1H,5H,6H,7H-pyrrolo[3,2-c]pyridin-4-one (23 mg, 27.49%) as a yellow solid.

LC-MS: (M+H)⁺ found: 438.20.

¹H NMR (300 MHz, DMSO-d6) δ 12.34 (s, 1H), 9.12 (s, 1H), 8.76 (s, 1H), 8.32 (d, J=8.1 Hz, 1H), 8.01 (s, 1H), 7.54 (s, 1H), 7.26 (s, 1H), 6.71-6.67 (m, 2H), 6.18 (d, J=6.9 Hz, 1H), 3.92 (s, 3H), 3.45 (s, 2H), 2.98 (s, 2H).

### Example 277. (7R)-3-[(3-chloro-2-methoxyphenyl)amino]-7-{[(2R)-4-methylmorpholin-2-yl]methyl}-2-(pyridin-4-yl)-1H,5H,6H,7H-pyrrolo[3,2-c]pyridin-4-one (Compound 646)

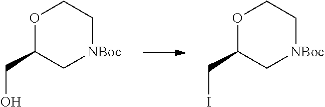 [SMILES: CC(C)(C)OC(=O)N1CCO[C@H](CO)C1 ; CC(C)(C)OC(=O)N1CCO[C@H](CI)C1]

Into a 500 mL 3-necked round-bottom flask were added tert-butyl (2S)-2-(hydroxymethyl)morpholine-4-carboxylate (20 g, 92.05 mmol, 1.00 equiv) and PPh₃ (31.39 g, 119.67 mmol, 1.30 equiv) and Imidazole (9.40 g, 138.08 mmol, 1.50 equiv) and DCM (200 mL) and followed by the addition of I2 (28.04 g, 110.46 mmol, 1.20 equiv) at room temperature. The resulting mixture was stirred for 5 h at room temperature under nitrogen atmosphere. The reaction was monitored by LCMS. The resulting mixture was concentrated under vacuum. The resulting mixture was diluted with ethyl ether (200 mL). The resulting mixture was filtered, the filter cake was washed with ethyl ether (3×50 mL). The filtrate was concentrated under reduced pressure. The residue was purified by silica gel column chromatography, eluted with PE/EA (10:1) to afford tert-butyl (2S)-2-(iodomethyl)morpholine-4-carboxylate (22.3 g, 74.05%) as a white solid.

LC-MS: (M+H)⁺ found 328.

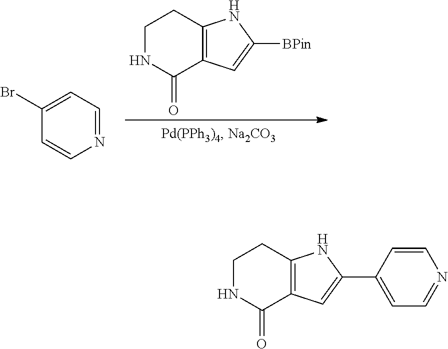

A solution of 4-bromopyridine (5 g, 31.65 mmol, 1.00 equiv) and 2-(4,4,5,5-tetramethyl-1,3,2-dioxaborolan-2-yl)-1H,5H,6H,7H-pyrrolo[3,2-c]pyridin-4-one (9.95 g, 37.98 mmol, 1.20 equiv) and Na2CO3 (10.16 g, 94.94 mmol, 3.00 equiv) and tetrakis(triphenylphosphine)palladium(0) (3.66 g, 3.17 mmol, 0.10 equiv) in dioxane (25.00 mL) and H₂O (5.00 mL). The mixture was stirred for overnight at 50 degrees C. under nitrogen atmosphere. The mixture was allowed to cool down to room temperature. The precipitated solids were collected by filtration and washed with water to afford 2-(pyridin-4-yl)-1H,5H,6H,7H-pyrrolo[3,2-c]pyridin-4-one (5 g, 74.09%) as a yellow solid.

LC-MS: (M+H)⁺ found 397

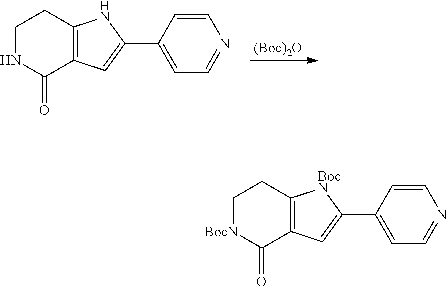 [SMILES: "(Boc)2O" ; O=C1NCCc2[nH]c(-c3ccncc3)cc21 ; CC(C)(C)OC(=O)N1CCc2c(cc(-c3ccncc3)n2C(=O)OC(C)(C)C)C1=O]

To a stirred solution of 2-(pyridin-4-yl)-1H,5H,6H,7H-pyrrolo[3,2-c]pyridin-4-one (6.7 g, 31.42 mmol, 1.00 equiv) and Boc20 (17.14 g, 78.55 mmol, 2.50 equiv) in THF (100 mL) was added TEA (9.54 g, 94.26 mmol, 3.00 equiv) and DMAP (384 mg, 3.14 mmol, 0.10 equiv). The resulting mixture was stirred for overnight at room temperature under nitrogen atmosphere. The resulting mixture was concentrated under reduced pressure. The residue was purified by silica gel column chromatography, eluted with PE/EA (10:1) to afford 1,5-di-tert-butyl 4-oxo-2-(pyridin-4-yl)-6H,7H-pyrrolo[3,2-c]pyridine-1,5-dicarboxylate (8.7 g, 66.97%) as a yellow solid.

LC-MS: (M+H)⁺ found: 414

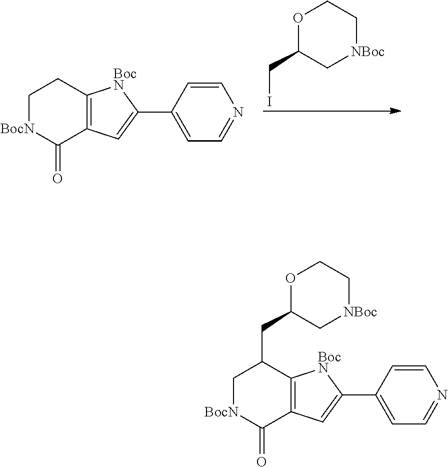

In to a 50 mL round-bottom flask, 1,5-di-tert-butyl 4-oxo-2-(pyridin-4-yl)-6H,7H-pyrrolo[3,2-c]pyridine-1,5-dicarboxylate (1.5 g, 3.63 mmol, 1.00 equiv) and tert-butyl (2S)-2-(iodomethyl)morpholine-4-carboxylate (4.75 g, 14.51 mmol, 4.00 equiv) in THF (30 mL) was added LiHMDS (5.44 mL, 5.44 mmol, 1.50 equiv) dropwise at −40 degrees C. under Ar2 atmosphere. The reaction mixture was stirred at −40 degrees C. for 5 h. The reaction was quenched with sat. NH4Cl (aq.) at −40° C. The resulting mixture was extracted with EtOAc (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with CH₂Clz/MeOH (10:1) to afford crude product. The residue was purified by reverse flash chromatography with the following conditions: column, C18 spherical column; mobile phase, ACN in water, 40% to 90°/% gradient in 30 min; detector, UV 254 nm. This resulted in 1,5-di-tert-butyl 7-{[(2R)-4-(tert-butoxycarbonyl)morpholin-2-yl]methyl}-4-oxo-2-(pyridin-4-yl)-6H,7H-pyrrolo[3,2-c]pyridine-1,5-dicarboxylate (450 mg, 20.24%) as a yellow solid.

LC-MS: (M+H)⁺ found 613.20

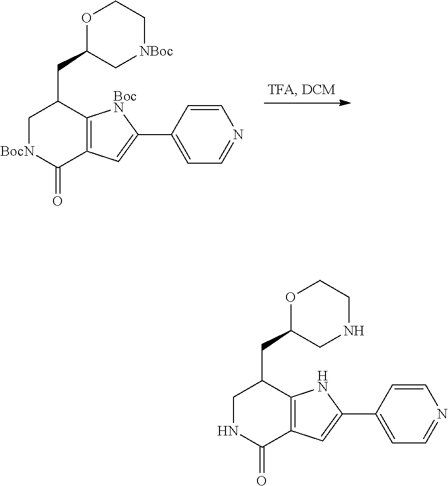 [SMILES: CC(C)(C)OC(=O)N1CCO[C@H](CC2CN(C(=O)OC(C)(C)C)C(=O)c3cc(-c4ccncc4)n(C(=O)OC(C)(C)C)c32)C1 ; O=C1NCC(C[C@@H]2CNCCO2)c2[nH]c(-c3ccncc3)cc21]

Into a 20 mL vial were added 1,5-di-tert-butyl 7-{[(2R)-4-(tert-butoxycarbonyl)morpholin-2-yl]methyl}-4-oxo-2-(pyridin-4-yl)-6H,7H-pyrrolo[3,2-c]pyridine-1,5-dicarboxylate (400 mg, 0.65 mmol, 1.00 equiv) and TFA (0.4 mL) in DCM (2 mL). The resulting mixture was stirred for overnight at room temperature under nitrogen atmosphere. The resulting mixture was concentrated under reduced pressure. The residue was dissolved in DMSO. The mixture was neutralized to pH 7 with DIEA. The residue was purified by reverse flash chromatography with the following conditions: column, C18 spherical column; mobile phase, ACN in water, 0% to 50% gradient in 30 min; detector, UV 254 nm. This resulted in 7-[(2R)-morpholin-2-ylmethyl]-2-(pyridin-4-yl)-1H,5H,6H,7H-pyrrolo[3,2-c]pyridin-4-one (192 mg, 94.15%) as a yellow solid.

LC-MS: (M+H)⁺ found: 313.00

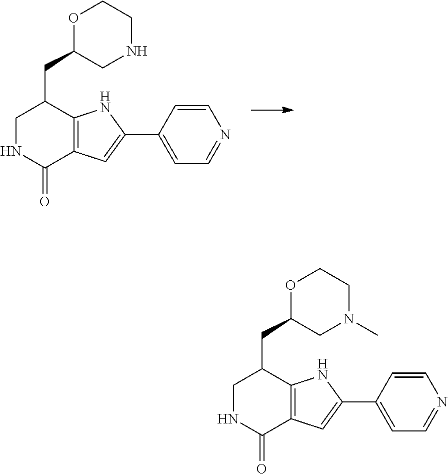 [SMILES: O=C1NCC(C[C@@H]2CNCCO2)c2[nH]c(-c3ccncc3)cc21 ; CN1CCO[C@H](CC2CNC(=O)c3cc(-c4ccncc4)[nH]c32)C1]

A solution of 7-[(2R)-morpholin-2-ylmethyl]-2-(pyridin-4-yl)-1H,5H,6H,7H-pyrrolo[3,2-c]pyridin-4-one (130 mg, 0.42 mmol, 1.00 equiv) in TFE (3 mL) was treated with formaldehyde (94 mg, 1.04 mmol, 2.50 equiv) for 30 min at room temperature under nitrogen atmosphere followed by the addition of NaBH₄ (31 mg, 0.83 mmol, 2.00 equiv) at 0° C. The resulting mixture was stirred for 2 h at room temperature under nitrogen atmosphere. The reaction was quenched with MeOH at 0° C. The residue was purified by Prep-TLC (CH₂Cl2/MeOH 10:1) to afford 7-{[(2R)-4-methylmorpholin-2-yl]methyl}-2-(pyridin-4-yl)-1H,5H,6H,7H-pyrrolo[3,2-c]pyridin-4-one (100 mg, 73.62%) as a white solid.

LC-MS: (M+H)⁺ found: 327.00.

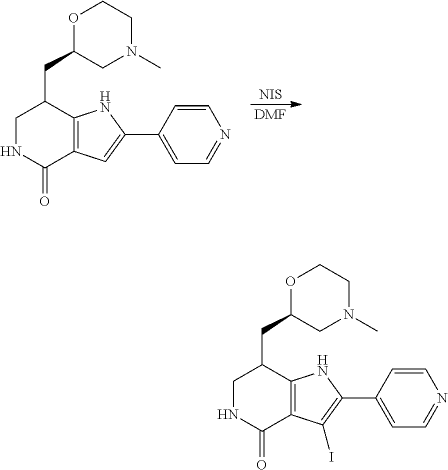

To a stirred mixture of 7-{[(2R)-4-methylmorpholin-2-yl]methyl}-2-(pyridin-4-yl)-1H,5H,6H,7H-pyrrolo[3,2-c]pyridin-4-one (100 mg, 0.31 mmol, 1.00 equiv) in ACN (0.3 mL) and DMF (0.3 mL) was added NIS (76 mg, 0.34 mmol, 1.10 equiv) at 0° C. under nitrogen atmosphere. The resulting mixture was stirred for Ih at room temperature under nitrogen atmosphere. The reaction was quenched with sat. Na2SO3 (aq.) at 0° C. The residue was purified by reverse flash chromatography with the following conditions: column, silica gel; mobile phase, MeCN in water, 10% to 50% gradient in 20 min; detector, UV 254 nm. This resulted in 3-iodo-7-{[(2R)-4-methylmorpholin-2-yl]methyl}-2-(pyridin-4-yl)-1H,5H,6H,7H-pyrrolo[3,2-c]pyridin-4-one (80 mg, 57.73%) as a yellow solid.

LC-MS: (M+H)⁺ found: 452.95.

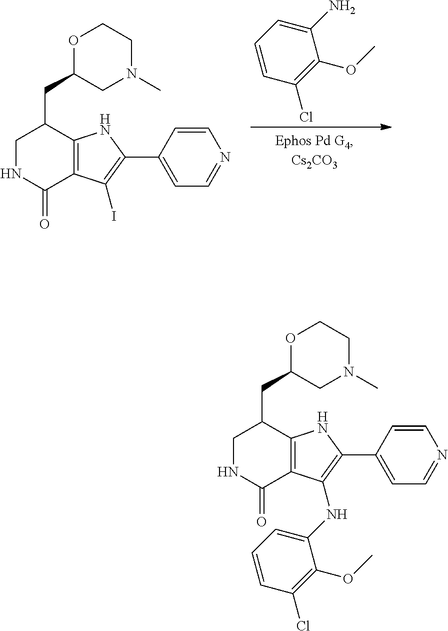

To a stirred mixture of 3-iodo-7-{[(2R)-4-methylmorpholin-2-yl]methyl}-2-(pyridin-4-yl)-1H,5H,6H,7H-pyrrolo[3,2-c]pyridin-4-one (80 mg, 0.18 mmol, 1.00 equiv) and 3-chloro-2-methoxyaniline (28 mg, 0.18 mmol, 1.00 equiv) in DMF (2 mL) were added EPhos Pd G4 (49 mg, 0.05 mmol, 0.30 equiv) and Cs₂CO₃ (115 mg, 0.35 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl2/MeOH (20:1) to afford 3-[(3-chloro-2-methoxyphenyl)amino]-7-{[(2R)-4-methylmorpholin-2-yl]methyl}-2-(pyridin-4-yl)-1H,5H,6H,7H-pyrrolo[3,2-c]pyridin-4-one (78 mg, 82.35%) as a yellow solid.

LC-MS: (M+H)⁺ found: 482.00.

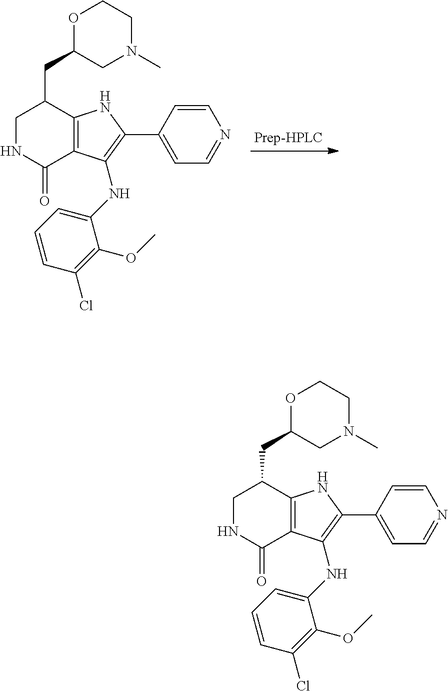 [SMILES: COc1c(Cl)cccc1Nc1c(-c2ccncc2)[nH]c2c1C(=O)NCC2C[C@@H]1CN(C)CCO1 ; COc1c(Cl)cccc1Nc1c(-c2ccncc2)[nH]c2c1C(=O)NC[C@@H]2C[C@@H]1CN(C)CCO1]

The crude product (78 mg) was purified by Prep-HPLC with the following conditions (Column: XBridge Prep Phenyl OBD Column, 19*250 mm, 5 μm; Mobile Phase A: Water (10 mmol/L NH4HCO3+0.1% NH3·H2O), Mobile Phase B: ACN; Flow rate: 25 m/min; Gradient: 28% B to 42% B in 11 min, 42% B; Wave Length: 254 nm; RT1 (min): 8.87) to afford (7R*)-3-[(3-chloro-2-methoxyphenyl)amino]-7-{[(2R)-4-methylmorpholin-2-yl]methyl}-2-(pyridin-4-yl)-1H,5H,6H,7H-pyrrolo[3,2-c]pyridin-4-one (12.6 mg, 16.11%) as a yellow solid.

LC-MS: (M+H)⁺ found: 482.05.

¹H NMR (400 MHz, Chloroform-d) δ 10.08 (s, 1H), 8.45-8.44 (m, 2H), 7.35 (s, 1H), 7.24-7.18 (m, 2H), 6.80-6.56 (m, 2H), 6.21-6.19 (m, 1H), 5.30 (s, 1H), 4.13-4.11 (m, 1H), 4.06 (s, 3H), 3.95-3.74 (m, 2H), 3.62-3.49 (m, 1H), 3.33-3.31 (m, 2H), 2.87-2.63 (m, 2H), 2.35 (s, 3H), 2.27-2.24 (m, 1H), 2.19-2.01 (m, 2H), 1.83-1.71 (m, 1H).

### Example 278. (7R)-3-[(3-chloro-2-methoxyphenyl)amino]-7-{[(2R)-4-methylmorpholin-2-yl]methyl}-2-(pyridin-4-yl)-1H,5H,6H,7H-pyrrolo[3,2-c]pyridin-4-one (Compound 645)

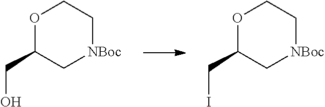 [SMILES: CC(C)(C)OC(=O)N1CCO[C@H](CO)C1 ; CC(C)(C)OC(=O)N1CCO[C@H](CI)C1]

Into a 500 mL 3-necked round-bottom flask were added tert-butyl (2S)-2-(hydroxymethyl)morpholine-4-carboxylate (20 g, 92.05 mmol, 1.00 equiv) and PPh₃ (31.39 g, 119.67 mmol, 1.30 equiv) and Imidazole (9.40 g, 138.08 mmol, 1.50 equiv) and DCM (200 mL) and followed by the addition of 12 (28.04 g, 110.46 mmol, 1.20 equiv) at room temperature. The resulting mixture was stirred for 5 h at room temperature under nitrogen atmosphere. The reaction was monitored by LCMS. The resulting mixture was concentrated under vacuum. The resulting mixture was diluted with ethyl ether (200 mL). The resulting mixture was filtered, the filter cake was washed with ethyl ether (3×50 mL). The filtrate was concentrated under reduced pressure. The residue was purified by silica gel column chromatography, eluted with PE/EA (10:1) to afford tert-butyl (2S)-2-(iodomethyl)morpholine-4-carboxylate (22.3 g, 74.05%) as a white solid.

LC-MS: (M+H)⁺ found 328.

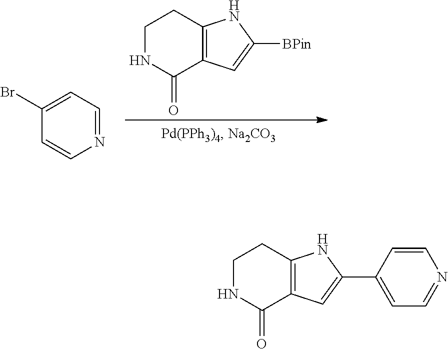

A solution of 4-bromopyridine (5 g, 31.65 mmol, 1.00 equiv) and 2-(4,4,5,5-tetramethyl-1,3,2-dioxaborolan-2-yl)-1H,5H,6H,7H-pyrrolo[3,2-c]pyridin-4-one (9.95 g, 37.98 mmol, 1.20 equiv) and Na2CO3 (10.16 g, 94.94 mmol, 3.00 equiv) and tetrakis(triphenylphosphine)palladium(0) (3.66 g, 3.17 mmol, 0.10 equiv) in dioxane (25.00 mL) and H2O (5.00 mL). The mixture was stirred for overnight at 50 degrees C. under nitrogen atmosphere. The mixture was allowed to cool down to room temperature. The precipitated solids were collected by filtration and washed with water to afford 2-(pyridin-4-yl)-1H,5H,6H,7H-pyrrolo[3,2-c]pyridin-4-one (5 g, 74.09%) as a yellow solid.

LC-MS: (M+H)⁺ found 397

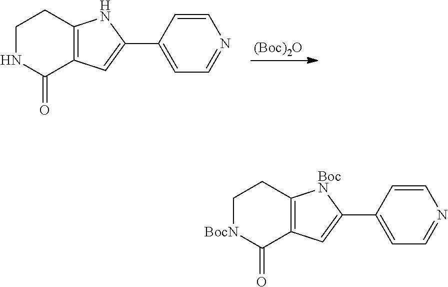 [SMILES: "(Boc)2O" ; O=C1NCCc2[nH]c(-c3ccncc3)cc21 ; CC(C)(C)OC(=O)N1CCc2c(cc(-c3ccncc3)n2C(=O)OC(C)(C)C)C1=O]

To a stirred solution of 2-(pyridin-4-yl)-1H,5H,6H,7H-pyrrolo[3,2-c]pyridin-4-one (6.7 g, 31.42 mmol, 1.00 equiv) and Boc20 (17.14 g, 78.55 mmol, 2.50 equiv) in THF (100 mL) was added TEA (9.54 g, 94.26 mmol, 3.00 equiv) and DMAP (384 mg, 3.14 mmol, 0.10 equiv). The resulting mixture was stirred for overnight at room temperature under nitrogen atmosphere. The resulting mixture was concentrated under reduced pressure. The residue was purified by silica gel column chromatography, eluted with PE/EA (10:1) to afford 1,5-di-tert-butyl 4-oxo-2-(pyridin-4-yl)-6H,7H-pyrrolo[3,2-c]pyridine-1,5-dicarboxylate (8.7 g, 66.97%) as a yellow solid.

LC-MS: (M+H)⁺ found: 414

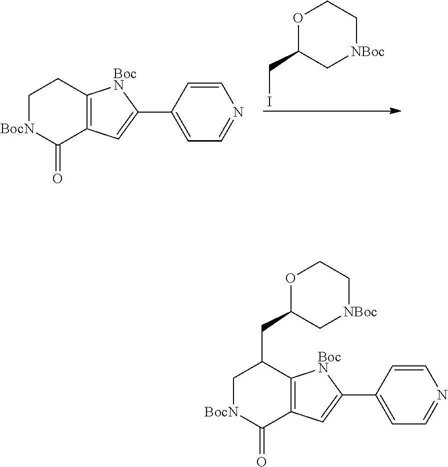

In to a 50 mL round-bottom flask, 1,5-di-tert-butyl 4-oxo-2-(pyridin-4-yl)-6H,7H-pyrrolo[3,2-c]pyridine-1,5-dicarboxylate (1.5 g, 3.63 mmol, 1.00 equiv) and tert-butyl (2S)-2-(iodomethyl)morpholine-4-carboxylate (4.75 g, 14.51 mmol, 4.00 equiv) in THF (30 mL) was added LiHMDS (5.44 mL, 5.44 mmol, 1.50 equiv) dropwise at −40 degrees C. under Ar₂ atmosphere. The reaction mixture was stirred at −40 degrees C. for 5 h. The reaction was quenched with sat. NH₄Cl (aq.) at −40° C. The resulting mixture was extracted with EtOAc (3×50 mL). The combined organic layers were washed with brine (2×30 mL), dried over anhydrous Na₂SO₄. After filtration, the filtrate was concentrated under reduced pressure. The residue was purified by silica gel column chromatography, eluted with CH₂Cl₂/MeOH (10:1) to afford crude product. The residue was purified by reverse flash chromatography with the following conditions: column, C18 spherical column; mobile phase, ACN in water, 40% to 90% gradient in 30 min; detector, UV 254 nm. This resulted in 1,5-di-tert-butyl 7-{[(2R)-4-(tert-butoxycarbonyl)morpholin-2-yl]methyl}-4-oxo-2-(pyridin-4-yl)-6H,7H-pyrrolo[3,2-c]pyridine-1,5-dicarboxylate (450 mg, 20.24%) as a yellow solid.

LC-MS: (M+H)⁺ found 613.20

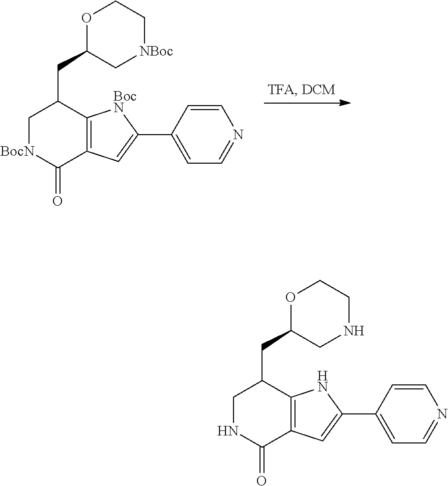 [SMILES: CC(C)(C)OC(=O)N1CCO[C@H](CC2CN(C(=O)OC(C)(C)C)C(=O)c3cc(-c4ccncc4)n(C(=O)OC(C)(C)C)c32)C1 ; O=C1NCC(C[C@@H]2CNCCO2)c2[nH]c(-c3ccncc3)cc21]

Into a 20 mL vial were added 1,5-di-tert-butyl 7-{[(2R)-4-(tert-butoxycarbonyl)morpholin-2-yl]methyl}-4-oxo-2-(pyridin-4-yl)-6H,7H-pyrrolo[3,2-c]pyridine-1,5-dicarboxylate (400 mg, 0.65 mmol, 1.00 equiv) and TFA (0.4 mL) in DCM (2 mL). The resulting mixture was stirred for overnight at room temperature under nitrogen atmosphere. The resulting mixture was concentrated under reduced pressure. The residue was dissolved in DMSO. The mixture was neutralized to pH 7 with DIEA. The residue was purified by reverse flash chromatography with the following conditions: column, C18 spherical column; mobile phase, ACN in water, 0% to 50% gradient in 30 min; detector, UV 254 nm. This resulted in 7-[(2R)-morpholin-2-ylmethyl]-2-(pyridin-4-yl)-1H,5H,6H,7H-pyrrolo[3,2-c]pyridin-4-one (192 mg, 94.15%) as a yellow solid.

LC-MS: (M+H)⁺ found: 313.00

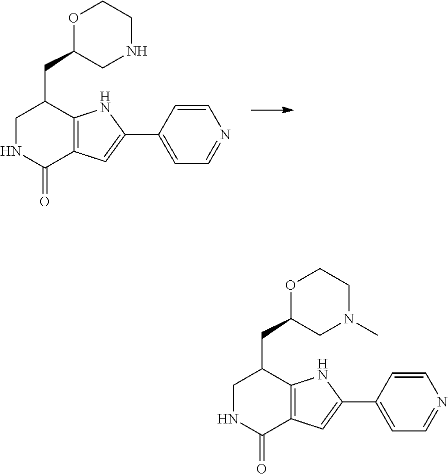 [SMILES: O=C1NCC(C[C@@H]2CNCCO2)c2[nH]c(-c3ccncc3)cc21 ; CN1CCO[C@H](CC2CNC(=O)c3cc(-c4ccncc4)[nH]c32)C1]

A solution of 7-[(2R)-morpholin-2-ylmethyl]-2-(pyridin-4-yl)-1H,5H,6H,7H-pyrrolo[3,2-c]pyridin-4-one (130 mg, 0.42 mmol, 1.00 equiv) in TFE (3 mL) was treated with formaldehyde (94 mg, 1.04 mmol, 2.50 equiv) for 30 min at room temperature under nitrogen atmosphere followed by the addition of NaBH4 (31 mg, 0.83 mmol, 2.00 equiv) at 0° C. The resulting mixture was stirred for 2 h at room temperature under nitrogen atmosphere. The reaction was quenched with MeOH at 0° C. The residue was purified by Prep-TLC (CH₂Cl₂/MeOH 10:1) to afford 7-{[(2R)-4-methylmorpholin-2-yl]methyl}-2-(pyridin-4-yl)-1H,5H,6H,7H-pyrrolo[3,2-c]pyridin-4-one (100 mg, 73.62%) as a white solid.

LC-MS: (M+H)⁺ found: 327.00.

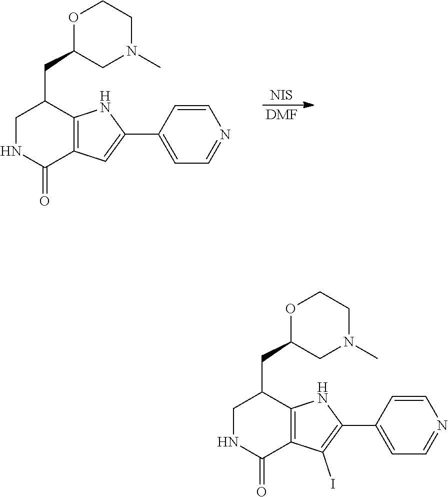

To a stirred mixture of 7-{[(2R)-4-methylmorpholin-2-yl]methyl}-2-(pyridin-4-yl)-1H,5H,6H,7H-pyrrolo[3,2-c]pyridin-4-one (100 mg, 0.31 mmol, 1.00 equiv) in ACN (0.3 mL) and DMF (0.3 mL) was added NIS (76 mg, 0.34 mmol, 1.10 equiv) at 0° C. under nitrogen atmosphere. The resulting mixture was stirred for Ih at room temperature under nitrogen atmosphere. The reaction was quenched with sat. Na2SO3 (aq.) at 0° C. The residue was purified by reverse flash chromatography with the following conditions: column, silica gel; mobile phase, MeCN in water, 10% to 50% gradient in 20 min; detector, UV 254 nm. This resulted in 3-iodo-7-{[(2R)-4-methylmorpholin-2-yl]methyl}-2-(pyridin-4-yl)-1H,5H,6H,7H-pyrrolo[3,2-c]pyridin-4-one (80 mg, 57.73%) as a yellow solid.

LC-MS: (M+H)⁺ found: 452.95.

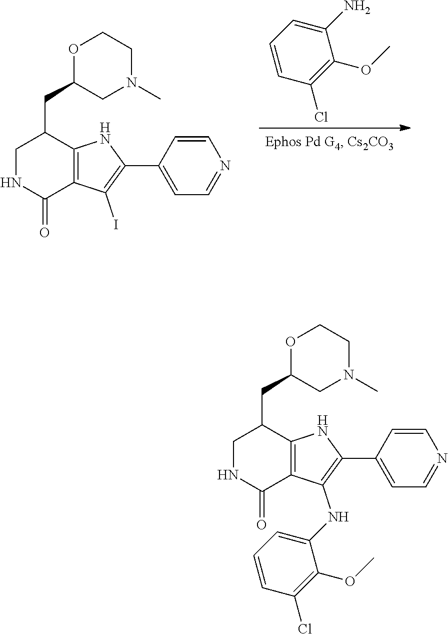

To a stirred mixture of 3-iodo-7-{[(2R)-4-methylmorpholin-2-yl]methyl}-2-(pyridin-4-yl)-1H,5H,6H,7H-pyrrolo[3,2-c]pyridin-4-one (80 mg, 0.18 mmol, 1.00 equiv) and 3-chloro-2-methoxyaniline (28 mg, 0.18 mmol, 1.00 equiv) in DMF (2 mL) were added EPhos Pd G4 (49 mg, 0.05 mmol, 0.30 equiv) and Cs₂CO₃ (115 mg, 0.35 mmol, 2.00 equiv) under argon atmosphere. The resulting suspension was backfilled with argon three times and stirred for 2 h at 50° C. LCMS confirmed completion of reaction and desired product was observed. The resulting mixture was filtered through a pad of silica and the filter cake was washed with DCM (2×10 mL). The filtrate was concentrated under reduced pressure that was purified by silica gel column chromatography, eluted with CH₂Cl₂/MeOH (20:1) to afford 3-[(3-chloro-2-methoxyphenyl)amino]-7-{[(2R)-4-methylmorpholin-2-yl]methyl}-2-(pyridin-4-yl)-1H,5H,6H,7H-pyrrolo[3,2-c]pyridin-4-one (78 mg, 82.35%) as a yellow solid.

LC-MS: (M+H)⁺ found: 482.00.

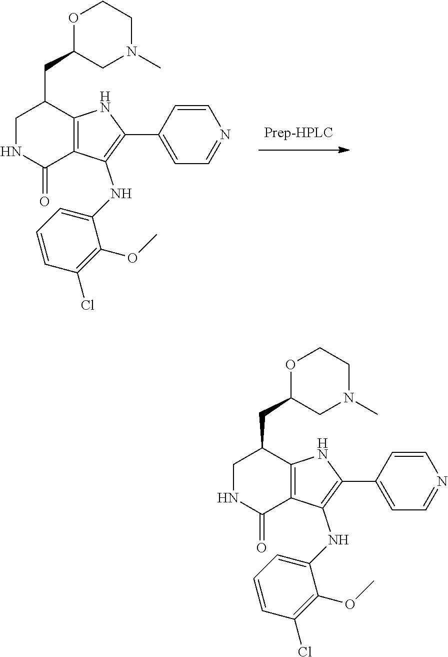 [SMILES: COc1c(Cl)cccc1Nc1c(-c2ccncc2)[nH]c2c1C(=O)NCC2C[C@@H]1CN(C)CCO1 ; COc1c(Cl)cccc1Nc1c(-c2ccncc2)[nH]c2c1C(=O)NC[C@H]2C[C@@H]1CN(C)CCO1]

The crude product (78 mg) was purified by Prep-HPLC with the following conditions (Column: XBridge Prep Phenyl OBD Column, 19*250 mm, 5 μm; Mobile Phase A: Water (10 mmol/L NH4HCO3+0.1% NH3·H2O), Mobile Phase B: ACN; Flow rate: 25 m/min; Gradient: 28% B to 42% B in 11 min, 42% B; Wave Length: 254 nm; RT1 (min): 8.87) to afford (7R)-3-[(3-chloro-2-methoxyphenyl)amino]-7-{[(2R)-4-methylmorpholin-2-yl]methyl}-2-(pyridin-4-yl)-1H,5H,6H,7H-pyrrolo[3,2-c]pyridin-4-one (8.4 mg, 10.74%) as a yellow solid.

LC-MS: (M+H)⁺ found: 482.05.

¹H NMR (400 MHz, Chloroform-d) δ 10.75 (s, 1H), 8.45-8.44 (m, 2H), 7.32 (s, 1H), 7.26-7.23 (m, 2H), 6.80-6.62 (m, 2H), 6.21-6.19 (m, 1H), 5.40 (s, 1H), 4.14-4.11 (m, 1H), 4.05 (s, 3H), 3.96-3.82 (m, 2H), 3.55-3.20 (m, 3H), 3.10-2.82 (m, 2H), 2.43 (s, 3H), 2.38-2.36 (m, 1H), 2.10-2.05 (m, 1H), 1.98-1.88 (m, 1H), 1.76-1.64 (m, 1H).

Bioactivity

### Example A. Inhibitor Activity on EGFR-Dependent Cell Growth

Cell lines are generated by transducing Ba/F3 cells with retroviruses containing vectors with EGFR WT, EGFR L858R, EGFR exon 19del, EGFR L858R/C797S, EGFR exon 20 NPG Ins D770_N771, EGFR exon 20 ASV Ins V769_D770, EGFR exon 20 SVD Ins D770_N771, or EGFR exon 20 FQEA Ins A763_V764 genes and a puromycin selection marker. Transduced cells are selected with puromycin for 7 days and are then be transferred into culture media without Interleukin 3 (IL3). EGFR WT cells are maintained with supplemental EGF. Surviving cells are confirmed to express EGFR by Western blot and maintained as a pool. The IC50 date were included in Table 6 and Table 7.

Study Design

1 Cell Seeding

1.1 Cells are harvested from flask into cell culture medium and the cell number counted.

1.2 Cells are diluted with culture medium to the desired density and 40 μL of cell suspension is added into each well of 384-well cell culture plate and the seeding density is 800 (FQEA, exon 19del), 600 (WT, NPG, L858R/C797S), or 400 (ASV, SVD, L858R) cells/well.

2 Compound Preparation and Treatment

2.1 Test compounds are dissolved to 10 mM in a DMSO stock solution. 45 μL of stock solution is transferred to a 384 polypropylene plate (pp-plate). Perform 3-fold, 10-point dilution via transferring 15 μL compound into 30 μL DMSO using a TECAN (EVO200) liquid handler.

2.2 Spin plates at room temperature at 1,000 RPM for 1 minute.

2.3 Transfer 120 nL of diluted compound from compound source plate into the cell plate.

2.4 After compound treatment for 72 hours, perform CTG detection for compound treatment plates as described in “Detection” section.

3 Detection

3.1 Plates are removed from incubators and equilibrated at room temperature for 15 minutes.

3.2 Thaw the CellTiter Glo reagents and allow to equilibrate to room temperature before the experiment.

3.3 Add 40 μL of CellTiter-Glo reagent into each well (at 1:1 to culture medium). Then place the plates at room temperature for 30 min followed by reading on EnVision.

4 Data Analysis

4.1 Inhibition activity is calculated following the formula below:

% Inhibition=100×(LumHC−LumSample)/(LumHC−LumLC)
where HC is obtained from cells treated with 0.1% DMSO only; and LC is obtained from culture medium only.

4.2 2. Calculate the IC₅₀ by fitting the Curve using Xlfit (v5.3.1.3), equation 201.

Y=Bottom+(Top−Bottom)/(1+10\hat{ }((Log IC₅₀−X)*HillSlope))

### Example B. Inhibitor Activity on EGFR phosphorylation (pEGFR)

EGFR mutant Ba/F3 cells were generated by transduction with retrovirus containing vectors expressing EGFR L858R, EGFR exon 19del, EGFR L858R/C797S, EGFR exon 20 NPG Ins D770_N771, EGFR exon 20 ASV Ins V769_D770, or EGFR exon 20 SVD Ins D770_N771 genes along with a puromycin selection marker. Transduced cells are selected with puromycin for 7 days and are then be transferred into culture media without Interleukin 3 (IL3). Surviving cells are confirmed to express EGFR by Western blot and maintained as a pool. CUTO14 cells were obtained from Dr. Robert C. Doebele at the University of Colorado. The IC50 date are included in Table 6 and Table 7.

Study Design

1 Cell Seeding

1.1 Cells are harvested from flask into cell culture medium and the cell number counted.

1.2 Cells are diluted with culture medium to the desired density and 40 μL of cell suspension is added into each well of 384-well cell culture plate and the seeding density is 50K cells/well (Ba/F3) or 12.5K cells/well (CUTO14).

2 Compound Preparation and Treatment

2.1 Test compounds are dissolved to 10 mM in a DMSO stock solution. 45 μL of stock solution is transferred to a 384 polypropylene plate (pp-plate). Perform 3-fold, 10-point dilution via transferring 15 μL compound into 30 μL DMSO using a TECAN (EVO200) liquid handler.

2.2 Spin plates at room temperature at 1,000 RPM for 1 minute.

2.3 Transfer 5 nL of diluted compound from compound source plate into the cell plate.

2.4 After compound treatment for 2 hours, perform pEGFR detection by AlphaLISA for compound treatment plates as described in “Detection” section.

3 Detection by pEGFR AlphaLISA (Perkin-Elmer)

3.1 Plates are removed from incubators and equilibrated at room temperature for 10 minutes, and media was removed

3.2 10 μL of lysis buffer is added and plates shaken at 600 rpm for 1 hr.

3.3 Prepare acceptor mix just before use and dispense 5 μL of acceptor mix to all the wells. Shake 350 rpm for Ihr in the dark

3.4 Prepare donor mix under low light conditions prior to use. Dispense 5 μL of donor mix to all the wells. Mix well on the shaker, seal and wrap in aluminum foil and incubate 1.5 hrs at room temperature in the dark

3.5 Transfer 18.5 μL mixture to OptiPlate 384, and read using an Envision. The IC50 date were included in Table 6 and Table 7.

TABLE 6
IC50 Data for EGFR Activity and Inhibitor Activity on EGFR phosphorylation (pEGFR)¹

| | BaF3 | BaF3 | BaF3 | BaF3 | BaF3 | ASV | SVD | NPG | CUTO14 |
|---|---|---|---|---|---|---|---|---|---|
| Com- | ASV | SVD | NPG | FQEA | WT | IC50 | IC50 | IC50 | IC50 |
| pound # | IC50 | IC50 | IC50 | IC50 | IC50 | (pEGFR) | (pEGFR) | (pEGFR) | (pEGFR) |
| 102 | ++ | ++ | ++ | ++ | + | +++ | NA | NA | NA |
| 108 | +++ | +++ | +++ | ++ | ++ | +++ | NA | +++ | NA |
| 109 | +++ | +++ | +++ | +++ | +++ | +++ | NA | +++ | NA |
| 110 | +++ | +++ | +++ | +++ | ++ | +++ | NA | +++ | NA |
| 112 | ++ | ++ | + | ++ | + | +++ | NA | NA | NA |
| 121 | +++ | +++ | +++ | NA | ++ | NA | NA | NA | NA |
| 138 | NA | NA | + | NA | + | NA | NA | + | NA |
| 148 | +++ | +++ | +++ | ++ | ++ | +++ | +++ | +++ | +++ |
| 149 | +++ | +++ | +++ | +++ | ++− | NA | NA | +++ | NA |
| 150 | NA | NA | + | NA | + | NA | NA | + | NA |
| 152 | NA | NA | +++ | NA | +++ | +++ | NA | +++ | NA |
| 154 | NA | NA | + | NA | +++ | + | NA | ++ | NA |
| 156 | + | + | + | + | + | + | NA | NA | NA |
| 157 | NA | NA | + | NA | ++ | NA | NA | + | NA |
| 158 | NA | NA | + | NA | + | NA | NA | ++ | NA |
| 159 | NA | NA | + | NA | ++ | NA | NA | + | NA |
| 160 | +++ | +++ | +++ | +++ | ++− | +++ | +++ | +++ | +++ |
| 162 | +++ | ++ | ++ | NA | ++ | NA | NA | NA | NA |
| 192 | + | + | NA | + | + | + | NA | NA | NA |
| 193 | ++ | +++ | NA | ++ | ++ | +++ | NA | NA | NA |
| 194 | ++ | ++ | NA | ++ | ++ | +++ | NA | NA | NA |
| 195 | ++ | ++ | ++ | ++ | ++ | +++ | NA | +++ | NA |
| 196 | + | + | NA | ++ | + | ++ | NA | NA | NA |
| 197 | + | + | NA | + | + | + | NA | NA | NA |
| 198 | + | + | NA | + | + | + | NA | NA | NA |
| 199 | + | + | ++ | ++ | + | ++ | NA | NA | NA |
| 200 | + | + | NA | + | + | ++ | NA | NA | NA |
| 201 | + | + | NA | + | + | + | NA | NA | NA |
| 202 | ++ | ++ | ++ | ++ | ++ | +++ | NA | +++ | NA |
| 203 | + | + | NA | ++ | + | + | NA | NA | NA |
| 204 | ++ | ++ | NA | ++ | ++ | ++ | NA | NA | NA |
| 206 | NA | NA | ++ | NA | ++ | ++ | NA | +++ | NA |
| 209 | ++ | ++ | NA | + | + | ++ | NA | NA | NA |
| 218 | ++ | ++ | + | ++ | + | +++ | NA | NA | NA |
| 219 | +++ | +++ | +++ | +++ | +++ | +++ | NA | NA | NA |
| 220 | ++ | ++ | ++ | ++ | + | ++ | NA | +++ | NA |
| 221 | ++ | ++ | ++ | ++ | + | ++ | NA | +++ | NA |
| 225 | ++ | ++ | ++ | + | + | ++ | NA | NA | NA |
| 239 | + | + | NA | + | + | NA | NA | NA | NA |
| 242 | + | + | NA | + | + | ++ | NA | NA | NA |
| 243 | ++ | ++ | NA | ++ | + | NA | NA | NA | NA |
| 244 | + | + | NA | + | + | ++ | NA | NA | NA |
| 246 | + | + | NA | + | + | + | NA | NA | NA |
| 252 | ++ | ++ | NA | ++ | + | NA | NA | NA | NA |
| 255 | ++ | ++ | NA | ++ | ++ | NA | NA | NA | NA |
| 259 | + | + | NA | + | + | NA | NA | NA | NA |
| 261 | + | + | NA | + | + | ++ | NA | NA | NA |
| 281 | ++ | ++ | +++ | ++ | + | NA | NA | +++ | NA |
| 282 | NA | NA | ++ | NA | ++ | +++ | NA | +++ | NA |
| 283 | ++ | ++ | ++ | +++ | ++ | ++ | NA | +++ | NA |
| 284 | + | + | NA | + | + | + | NA | NA | NA |
| 285 | ++ | ++ | ++ | ++ | + | +++ | NA | +++ | NA |
| 286 | + | + | + | + | + | + | NA | ++ | NA |
| 288 | + | + | NA | + | + | + | NA | NA | NA |
| 289 | + | + | + | + | + | + | NA | NA | NA |
| 290 | +++ | +++ | ++ | +++ | ++ | +++ | NA | +++ | NA |
| 292 | ++ | ++ | ++ | ++ | + | +++ | NA | NA | NA |
| 293 | + | + | NA | + | + | + | NA | NA | NA |
| 295 | ++ | + | + | ++ | + | ++ | NA | NA | NA |
| 297 | +++ | +++ | ++ | ++ | + | +++ | NA | NA | NA |
| 299 | ++ | ++ | ++ | ++ | + | NA | NA | +++ | NA |
| 301 | + | + | NA | + | + | + | NA | NA | NA |
| 302 | + | + | NA | + | + | + | NA | NA | NA |
| 303 | + | ++ | NA | + | + | ++ | NA | NA | NA |
| 304 | ++ | ++ | NA | ++ | + | +++ | NA | NA | NA |
| 305 | ++ | ++ | NA | ++ | + | +++ | NA | NA | NA |
| 306 | + | + | NA | ++ | + | ++ | NA | NA | NA |
| 307 | + | + | NA | + | + | ++ | NA | NA | NA |
| 308 | ++ | ++ | ++ | ++ | + | +++ | NA | +++ | NA |
| 316 | +++ | +++ | NA | +++ | ++ | NA | NA | NA | NA |
| 323 | +++ | +++ | NA | +++ | ++ | NA | NA | NA | NA |
| 379 | +++ | +++ | NA | +++ | ++ | NA | NA | NA | NA |
| 408 | +++ | +++ | +++ | +++ | +++ | +++ | +++ | +++ | +++ |
| 409 | +++ | +++ | NA | +++ | ++ | NA | NA | NA | NA |
| 410 | ++ | ++ | NA | ++ | + | NA | NA | NA | NA |
| 413 | +++ | +++ | N.A | +++ | +++ | NA | NA | NA | NA |
| 417 | ++ | ++ | NA | ++ | + | NA | NA | NA | NA |
| 418 | ++ | ++ | NA | ++ | + | NA | NA | NA | NA |
| 440 | +++ | +++ | NA | +++ | ++ | NA | NA | NA | NA |
| 441 | +++ | +++ | +++ | +++ | ++ | NA | NA | +++ | NA |
| 442 | +++ | +++ | +++ | +++ | ++ | NA | NA | +++ | NA |
| 470 | +++ | +++ | NA | ++ | ++ | NA | NA | NA | NA |
| 471 | ++ | ++ | NA | ++ | + | NA | NA | NA | NA |
| 472 | ++ | ++ | NA | + | + | NA | NA | NA | NA |
| 473 | ++ | ++ | NA | ++ | + | NA | NA | NA | NA |
| 474 | + | + | NA | + | + | NA | NA | NA | NA |
| 475 | + | + | NA | + | + | NA | NA | NA | NA |
| 476 | + | + | NA | + | + | NA | NA | NA | NA |
| 477 | +++ | +++ | NA | +++ | ++ | NA | NA | NA | NA |
| 478 | +++ | +++ | +++ | NA | +++ | NA | NA | NA | NA |
| 479 | ++ | ++ | NA | ++ | + | NA | NA | NA | NA |
| 480 | +++ | +++ | +++ | +++ | ++ | NA | NA | NA | NA |
| 481 | +++ | +++ | +++ | +++ | ++ | +++ | +++ | +++ | +++ |
| 482 | +++ | +++ | +++ | +++ | +++ | +++ | +++ | +++ | +++ |
| 483 | +++ | +++ | +++ | ++ | + | NA | NA | NA | +++ |
| 484 | + | + | NA | + | + | NA | NA | NA | NA |
| 485 | + | + | NA | + | + | NA | NA | NA | NA |
| 486 | + | + | NA | + | + | NA | NA | NA | NA |
| 487 | ++ | ++ | NA | ++ | + | NA | NA | NA | NA |
| 488 | +++ | +++ | +++ | ++ | + | NA | NA | NA | NA |
| 489 | ++ | ++ | NA | ++ | + | NA | NA | NA | NA |
| 490 | + | + | + | NA | + | NA | NA | NA | NA |
| 491 | + | + | NA | + | + | NA | NA | NA | NA |
| 492 | + | + | NA | + | + | NA | NA | NA | NA |
| 493 | ++ | ++ | NA | ++ | + | NA | NA | NA | NA |
| 494 | + | + | NA | + | + | NA | NA | NA | NA |
| 495 | +++ | +++ | NA | +++ | + | NA | NA | NA | NA |
| 496 | ++ | ++ | NA | ++ | + | NA | NA | NA | NA |
| 497 | ++ | ++ | +++ | NA | ++ | NA | NA | NA | NA |
| 498 | + | + | ++ | NA | + | NA | NA | NA | NA |
| 499 | + | + | + | NA | + | NA | NA | NA | NA |
| 500 | + | + | + | NA | + | NA | NA | NA | NA |
| 501 | +++ | +++ | NA | +++ | ++ | NA | NA | NA | NA |
| 502 | ++ | ++ | ++ | ++ | + | NA | NA | +++ | NA |
| 503 | +++ | +++ | NA | +++ | ++ | NA | NA | NA | NA |
| 504 | +++ | +++ | NA | ++ | ++ | NA | NA | NA | NA |
| 505 | +++ | +++ | NA | ++ | + | NA | NA | NA | NA |
| 506 | ++ | ++ | NA | ++ | + | NA | NA | NA | NA |
| 507 | +++ | +++ | NA | +++ | ++ | NA | NA | NA | NA |
| 508 | + | ++ | NA | ++ | + | NA | NA | NA | NA |
| 509 | +++ | +++ | +++ | +++ | +++ | NA | NA | NA | NA |
| 510 | +++ | +++ | NA | ++ | + | NA | NA | NA | NA |
| 511 | +++ | +++ | +++ | +++ | ++ | NA | NA | +++ | NA |
| 512 | +++ | +++ | +++ | +++ | ++ | +++ | +++ | +++ | +++ |
| 513 | +++ | +++ | NA | +++ | +++ | NA | NA | NA | NA |
| 514 | +++ | +++ | ++ | +++ | +++ | +++ | +++ | +++ | ++ |
| 515 | +++ | +++ | +++ | +++ | +++ | +++ | +++ | +++ | +++ |
| 516 | +++ | +++ | NA | +++ | ++ | NA | NA | NA | NA |
| 517 | +++ | +++ | NA | ++ | ++ | NA | NA | NA | NA |
| 518 | +++ | +++ | NA | +++ | ++ | NA | NA | NA | NA |
| 519 | ++ | ++ | ++ | NA | + | NA | NA | NA | NA |
| 520 | ++ | ++ | NA | +++ | ++ | NA | NA | NA | NA |
| 521 | ++ | ++ | NA | ++ | + | NA | NA | NA | NA |
| 522 | ++ | ++ | ++ | NA | + | NA | NA | NA | NA |
| 523 | ++ | +++ | +++ | NA | ++ | NA | NA | NA | NA |
| 524 | +++ | +++ | +++ | NA | ++ | +++ | +++ | +++ | +++ |
| 525 | +++ | +++ | +++ | +++ | ++ | +++ | +++ | +++ | +++ |
| 526 | ++ | ++ | ++ | ++ | + | NA | NA | NA | NA |
| 527 | +++ | +++ | +++ | NA | ++ | NA | NA | NA | +++ |
| 528 | ++ | ++ | +++ | NA | + | NA | NA | NA | NA |
| 529 | ++ | ++ | ++ | NA | + | NA | NA | NA | NA |
| 530 | +++ | +++ | +++ | NA | ++ | NA | NA | NA | NA |
| 531 | +++ | +++ | +++ | +++ | ++ | NA | NA | NA | +++ |
| 532 | +++ | +++ | +++ | NA | + | NA | NA | +++ | NA |
| 537 | +++ | +++ | +++ | NA | ++ | NA | NA | NA | NA |
| 538 | +++ | +++ | +++ | NA | ++ | NA | NA | NA | NA |
| 539 | ++ | ++ | ++ | NA | + | NA | NA | NA | NA |
| 542 | + | + | ++ | NA | + | NA | NA | NA | NA |
| 543 | ++ | +++ | +++ | NA | + | NA | NA | NA | NA |
| 544 | ++ | ++ | NA | ++ | + | NA | NA | NA | NA |
| 545 | ++ | ++ | +++ | NA | + | NA | NA | NA | NA |
| 546 | + | ++ | ++ | NA | + | NA | NA | NA | NA |
| 551 | +++ | +++ | +++ | ++ | ++ | +++ | +++ | +++ | +++ |
| 552 | +++ | +++ | +++ | NA | + | NA | NA | NA | +++ |
| 553 | +++ | +++ | NA | +++ | ++ | NA | NA | NA | NA |
| 554 | +++ | +++ | +++ | NA | +++ | NA | NA | NA | NA |
| 555 | + | + | + | NA | + | NA | NA | NA | NA |
| 556 | +++ | +++ | +++ | NA | ++ | NA | NA | NA | NA |
| 557 | +++ | +++ | +++ | NA | +++ | NA | NA | +++ | NA |
| 558 | +++ | +++ | +++ | NA | +++ | NA | NA | NA | NA |
| 559 | +++ | +++ | +++ | +++ | +++ | NA | NA | NA | +++ |
| 560 | +++ | +++ | +++ | NA | ++ | NA | NA | NA | NA |
| 561 | +++ | +++ | +++ | NA | ++ | NA | NA | NA | NA |
| 562 | +++ | +++ | +++ | NA | ++ | NA | NA | NA | +++ |
| 563 | ++ | ++ | ++ | NA | + | NA | NA | NA | NA |
| 564 | ++ | ++ | ++ | NA | + | NA | NA | NA | NA |
| 565 | ++ | ++ | ++ | NA | + | NA | NA | NA | NA |
| 566 | +++ | +++ | +++ | +++ | +++ | +++ | +++ | +++ | +++ |
| 567 | +++ | +++ | +++ | +++ | ++ | +++ | +++ | +++ | +++ |
| 568 | +++ | +++ | +++ | +++ | ++ | NA | NA | NA | +++ |
| 569 | +++ | +++ | +++ | +++ | ++ | NA | NA | NA | +++ |
| 570 | +++ | +++ | +++ | +++ | ++ | +++ | +++ | +++ | +++ |
| 571 | +++ | +++ | +++ | +++ | ++ | NA | NA | NA | +++ |
| 572 | ++ | +++ | ++ | NA | + | NA | NA | NA | NA |
| 573 | +++ | +++ | +++ | NA | ++ | NA | NA | +++ | NA |
| 574 | +++ | +++ | +++ | NA | + | NA | NA | NA | NA |
| 575 | +++ | +++ | +++ | NA | ++ | NA | NA | NA | NA |
| 576 | +++ | +++ | +++ | NA | +++ | NA | NA | +++ | NA |
| 577 | +++ | +++ | +++ | NA | ++ | NA | NA | NA | NA |
| 585 | +++ | +++ | +++ | NA | ++ | NA | NA | NA | NA |
| 586 | +++ | +++ | +++ | NA | ++ | NA | NA | NA | NA |
| 587 | +++ | +++ | +++ | NA | ++ | NA | NA | NA | NA |
| 588 | +++ | +++ | +++ | NA | +++ | NA | NA | NA | NA |
| 589 | +++ | +++ | +++ | NA | ++ | NA | NA | NA | NA |
| 590 | + | + | ++ | NA | + | NA | NA | NA | NA |
| 591 | +++ | +++ | +++ | NA | ++ | NA | NA | NA | NA |
| 592 | +++ | +++ | +++ | NA | ++ | NA | NA | NA | +++ |
| 593 | +++ | +++ | +++ | NA | ++ | NA | NA | NA | +++ |
| 594 | +++ | +++ | +++ | NA | ++ | NA | NA | NA | NA |
| 595 | ++ | ++ | ++ | NA | ++ | NA | NA | NA | NA |
| 603 | +++ | +++ | +++ | NA | + | NA | NA | NA | NA |
| 604 | +++ | +++ | +++ | +++ | ++ | NA | NA | NA | +++ |
| 605 | ++ | ++ | ++ | NA | + | NA | NA | NA | NA |
| 606 | +++ | +++ | +++ | +++ | ++ | NA | NA | NA | +++ |
| 607 | + | + | + | NA | + | NA | NA | NA | NA |
| 608 | + | + | + | NA | + | NA | NA | NA | NA |
| 609 | ++ | ++ | + | NA | + | NA | NA | NA | NA |
| 610 | +++ | +++ | +++ | NA | ++ | NA | NA | NA | +++ |
| 611 | +++ | +++ | +++ | NA | +++ | NA | NA | NA | +++ |
| 612 | +++ | +++ | +++ | NA | ++ | NA | NA | NA | +++ |
| 641 | +++ | +++ | +++ | NA | ++ | NA | NA | NA | +++ |
| 642 | +++ | +++ | +++ | NA | ++ | NA | NA | NA | NA |
| 643 | +++ | +++ | +++ | NA | +++ | NA | NA | NA | +++ |
| 644 | +++ | +++ | +++ | NA | +++ | NA | NA | NA | +++ |
| 645 | +++ | +++ | +++ | NA | + | NA | NA | NA | +++ |
| 646 | + | + | + | NA | + | NA | NA | NA | +++ |
| 649 | ++ | ++ | ++ | NA | + | NA | NA | NA | ++ |
| 650 | ++ | ++ | ++ | NA | + | NA | NA | NA | +++ |
| 651 | +++ | +++ | +++ | NA | ++ | NA | NA | NA | +++ |
| 652 | ++ | ++ | ++ | NA | + | NA | NA | NA | +++ |
| 653 | ++ | ++ | ++ | NA | + | NA | NA | NA | NA |
| 654 | +++ | +++ | +++ | NA | ++ | NA | NA | NA | NA |
| 655 | +++ | +++ | +++ | NA | ++ | NA | NA | NA | +++ |
| 656 | +++ | +++ | +++ | NA | + | NA | NA | NA | +++ |
| 657 | +++ | +++ | +++ | NA | ++ | NA | NA | NA | +++ |
| 658 | +++ | +++ | +++ | NA | ++ | NA | NA | NA | +++ |
| 659 | +++ | +++ | +++ | NA | + | NA | NA | NA | NA |
| 675 | ++ | ++ | NA | ++ | + | NA | NA | NA | NA |
| 676 | NA | NA | + | NA | + | NA | NA | NA | NA |
| 677 | +++ | +++ | NA | +++ | +++ | NA | NA | NA | NA |
| 678 | +++ | +++ | +++ | +++ | +++ | +++ | NA | +++ | NA |
| 679 | +++ | +++ | +++ | +++ | +++ | NA | NA | NA | NA |
| 680 | +++ | +++ | +++ | +++ | +++ | +++ | NA | +++ | NA |
| 681 | +++ | +++ | +++ | +++ | +++ | NA | NA | +++ | NA |
| 682 | NA | NA | +++ | NA | +++ | NA | NA | +++ | NA |
| 683 | +++ | +++ | +++ | +++ | ++ | NA | NA | NA | NA |
| 684 | +++ | +++ | +++ | +++ | ++ | NA | NA | +++ | NA |
| 685 | NA | NA | ++ | NA | ++ | +++ | NA | +++ | NA |
| 686 | +++ | +++ | NA | ++ | ++ | +++ | NA | NA | NA |
| 687 | ++ | ++ | ++ | ++ | + | ++ | NA | +++ | NA |
| 688 | + | + | + | + | + | + | NA | NA | NA |
| 689 | + | + | --- | ++ | NA | ++ | NA | NA | NA |
| 690 | NA | NA | ++ | NA | +++ | +++ | NA | +++ | NA |
| 691 | ++ | ++ | ++ | ++ | ++ | +++ | NA | +++ | NA |
| 692 | ++ | ++ | ++ | ++ | ++ | ++ | NA | +++ | NA |
| 693 | + | + | NA | + | + | + | NA | NA | NA |
| 694 | +++ | +++ | +++ | +++ | +++ | +++ | NA | NA | NA |
| 695 | +++ | +++ | NA | +++ | +++ | +++ | NA | NA | NA |
| 696 | +++ | +++ | +++ | +++ | +++ | +++ | NA | NA | NA |
| 697 | +++ | +++ | +++ | +++ | +++ | +++ | NA | NA | NA |
| 698 | ++ | + | ++ | ++ | + | ++ | NA | NA | NA |
| 699 | + | + | NA | + | + | ++ | NA | NA | NA |
| 700 | + | + | NA | + | + | ++ | NA | NA | NA |
| 701 | + | + | + | + | + | ++ | NA | NA | NA |
| 702 | ++ | ++ | ++ | ++ | + | ++ | NA | NA | NA |
| 703 | + | + | NA | + | + | ++ | NA | NA | NA |
| 704 | + | + | NA | + | + | + | NA | NA | NA |
| 705 | + | + | NA | + | + | + | NA | NA | NA |
| 706 | +++ | +++ | +++ | ++ | ++ | +++ | NA | NA | NA |
| 707 | + | + | + | + | + | + | NA | NA | NA |
| 708 | + | + | NA | + | + | NA | NA | NA | NA |
| 709 | +++ | +++ | NA | ++ | ++ | NA | NA | NA | NA |
| 710 | ++ | ++ | ++ | ++ | + | +++ | NA | NA | NA |
| 711 | + | + | NA | + | + | ++ | NA | NA | NA |
| 712 | + | + | NA | + | + | ++ | NA | NA | NA |
| 713 | ++ | ++ | ++ | + | + | NA | NA | NA | NA |
| 714 | ++ | ++ | +++ | ++ | + | NA | NA | NA | NA |
| 715 | ++ | ++ | NA | ++ | + | +++ | NA | NA | NA |
| 716 | ++ | ++ | ++ | ++ | + | NA | NA | NA | NA |
| 717 | ++ | ++ | NA | ++ | ++ | +++ | NA | NA | NA |
| 718 | + | + | NA | + | + | ++ | NA | NA | NA |
| 719 | ++ | + | ++ | + | + | NA | NA | NA | NA |
| 720 | + | + | NA | + | + | NA | NA | NA | NA |
| 721 | + | + | + | + | + | ++ | NA | NA | NA |
| 722 | + | + | + | + | + | ++ | NA | NA | NA |
| 723 | ++ | + | NA | + | + | NA | NA | NA | NA |
| 724 | ++ | ++ | NA | + | + | NA | NA | NA | NA |
| 725 | ++ | ++ | +++ | ++ | ++ | +++ | NA | NA | NA |
| 726 | +++ | +++ | +++ | +++ | +++ | +++ | +++ | +++ | +++ |
| 727 | +++ | +++ | +++ | +++ | ++ | NA | NA | NA | NA |
| 728 | + | + | NA | + | + | + | NA | NA | NA |
| 729 | ++ | +++ | NA | ++ | ++ | NA | NA | NA | NA |
| 730 | +++ | +++ | +++ | +++ | +++ | NA | NA | +++ | NA |
| 731 | +++ | +++ | +++ | NA | +++ | NA | NA | +++ | NA |
| 732 | +++ | +++ | +++ | +++ | +++ | NA | NA | NA | NA |
| 733 | +++ | +++ | NA | +++ | ++ | NA | NA | NA | NA |
| 734 | +++ | +++ | +++ | +++ | ++ | NA | NA | NA | NA |
| 735 | +++ | +++ | +++ | +++ | ++ | NA | NA | NA | NA |
| 736 | +++ | +++ | ++ | ++ | ++ | NA | NA | NA | NA |
| 737 | +++ | +++ | +++ | +++ | ++ | NA | NA | NA | NA |
| 738 | +++ | +++ | +++ | ++ | ++ | NA | NA | NA | NA |
| 739 | +++ | +++ | +++ | ++ | ++ | NA | NA | NA | NA |
| 740 | +++ | +++ | NA | +++ | +++ | NA | NA | NA | NA |
| 741 | +++ | +++ | +++ | +++ | +++ | NA | NA | NA | NA |
| 742 | +++ | +++ | +++ | +++ | ++ | NA | NA | NA | NA |
| 743 | +++ | +++ | +++ | +++ | ++ | NA | NA | NA | NA |
| 744 | +++ | +++ | NA | +++ | +++ | NA | NA | NA | NA |
| 745 | +++ | +++ | NA | +++ | +++ | NA | NA | NA | NA |
| 746 | +++ | +++ | +++ | +++ | ++ | NA | NA | NA | NA |
| 747 | +++ | +++ | +++ | +++ | +++ | +++ | +++ | +++ | +++ |
| 748 | ++ | ++ | NA | ++ | + | NA | NA | NA | NA |
| 749 | +++ | +++ | +++ | +++ | ++ | NA | NA | NA | NA |
| 750 | +++ | +++ | +++ | +++ | ++ | +++ | +++ | +++ | +++ |
| 751 | ++ | ++ | NA | ++ | + | NA | NA | NA | NA |
| 752 | +++ | +++ | NA | +++ | +++ | NA | NA | NA | NA |
| 753 | +++ | +++ | +++ | +++ | +++ | NA | NA | NA | NA |
| 754 | +++ | +++ | +++ | +++ | +++ | NA | NA | NA | NA |
| 755 | +++ | +++ | NA | +++ | ++ | NA | NA | NA | NA |
| 756 | +++ | ++ | NA | ++ | ++ | NA | NA | NA | NA |
| 757 | +++ | +++ | ++ | +++ | ++ | NA | NA | +++ | NA |
| 758 | ++ | ++ | ++ | ++ | ++ | NA | NA | NA | NA |
| 759 | +++ | +++ | NA | +++ | ++ | NA | NA | NA | NA |
| 760 | +++ | +++ | NA | +++ | +++ | NA | NA | NA | NA |
| 761 | +++ | +++ | +++ | +++ | +++ | NA | NA | NA | NA |
| 762 | +++ | +++ | NA | +++ | +++ | NA | NA | NA | NA |
| 763 | +++ | +++ | ++ | ++ | ++ | NA | NA | NA | NA |
| 764 | +++ | +++ | NA | +++ | +++ | NA | NA | NA | NA |
| 765 | +++ | +++ | NA | +++ | +++ | NA | NA | NA | NA |
| 766 | +++ | +++ | +++ | +++ | ++ | NA | NA | NA | NA |
| 767 | +++ | +++ | +++ | +++ | +++ | NA | NA | NA | NA |
| 768 | +++ | +++ | NA | +++ | ++ | NA | NA | NA | NA |
| 769 | +++ | +++ | NA | +++ | +++ | NA | NA | NA | NA |
| 770 | +++ | +++ | NA | +++ | +++ | NA | NA | NA | NA |
| 771 | +++ | +++ | NA | +++ | +++ | NA | NA | NA | NA |
| 772 | + | + | NA | + | + | NA | NA | NA | NA |
| 773 | + | + | NA | + | + | NA | NA | NA | NA |
| 774 | +++ | +++ | NA | +++ | +++ | NA | NA | NA | NA |
| 775 | +++ | +++ | NA | +++ | +++ | NA | NA | NA | NA |
| 776 | +++ | +++ | NA | +++ | ++ | NA | NA | NA | NA |
| 777 | +++ | +++ | NA | +++ | ++ | NA | NA | NA | NA |
| 778 | ++ | ++ | NA | ++ | + | NA | NA | NA | NA |
| 779 | +++ | +++ | +++ | +++ | ++ | NA | NA | NA | NA |
| 780 | +++ | +++ | +++ | +++ | ++ | NA | NA | NA | NA |
| 781 | ++ | ++ | NA | ++ | + | NA | NA | NA | NA |
| 782 | ++ | ++ | NA | ++ | + | NA | NA | NA | NA |
| 783 | +++ | +++ | NA | ++ | + | NA | NA | NA | NA |
| 784 | ++ | ++ | NA | ++ | + | NA | NA | NA | NA |
| 785 | +++ | +++ | NA | +++ | ++ | NA | NA | NA | NA |
| 786 | +++ | +++ | NA | ++ | ++ | NA | NA | NA | NA |
| 787 | +++ | +++ | +++ | +++ | +++ | NA | NA | NA | NA |
| 788 | +++ | +++ | +++ | +++ | +++ | NA | NA | NA | NA |
| 789 | + | + | NA | + | + | NA | NA | NA | NA |
| 790 | +++ | +++ | +++ | NA | ++ | NA | NA | NA | +++ |
| 791 | +++ | +++ | NA | ++ | ++ | NA | NA | NA | NA |
| 792 | +++ | +++ | NA | +++ | ++ | NA | NA | NA | NA |
| 793 | +++ | +++ | NA | +++ | ++ | NA | NA | NA | NA |
| 794 | +++ | +++ | NA | +++ | ++ | NA | NA | NA | NA |
| 795 | ++ | ++ | NA | ++ | + | NA | NA | NA | NA |
| 796 | + | + | NA | + | + | NA | NA | NA | NA |
| 797 | + | + | NA | + | + | NA | NA | NA | NA |
| 798 | +++ | +++ | NA | +++ | ++ | NA | NA | NA | NA |
| 799 | +++ | +++ | NA | +++ | ++ | NA | NA | NA | NA |
| 800 | +++ | +++ | NA | ++ | ++ | NA | NA | NA | NA |
| 801 | ++ | + | NA | + | + | NA | NA | NA | NA |
| 802 | + | + | NA | + | + | NA | NA | NA | NA |
| 803 | +++ | +++ | NA | +++ | ++ | NA | NA | NA | NA |
| 804 | ++ | +++ | NA | +++ | ++ | NA | NA | NA | NA |
| 805 | ++ | +++ | NA | +++ | ++ | NA | NA | NA | NA |
| 806 | + | + | NA | ++ | + | NA | NA | NA | NA |
| 807 | +++ | +++ | NA | +++ | +++ | NA | NA | NA | NA |
| 808 | +++ | +++ | NA | +++ | +++ | NA | NA | +++ | NA |
| 809 | +++ | +++ | NA | +++ | ++ | NA | NA | NA | NA |
| 810 | +++ | +++ | NA | +++ | ++ | NA | NA | NA | NA |
| 811 | + | + | NA | + | + | NA | NA | NA | NA |
| 812 | ++ | ++ | NA | ++ | + | NA | NA | NA | NA |
| 813 | ++ | ++ | NA | ++ | + | NA | NA | NA | NA |
| 814 | +++ | +++ | NA | +++ | +++ | NA | NA | NA | NA |
| 815 | ++ | ++ | NA | ++ | + | NA | NA | NA | NA |
| 816 | ++ | ++ | NA | ++ | + | NA | NA | NA | NA |
| 817 | +++ | +++ | +++ | +++ | ++ | +++ | +++ | +++ | +++ |
| 818 | ++ | ++ | NA | ++ | + | NA | NA | NA | NA |
| 819 | ++ | ++ | NA | ++ | + | NA | NA | NA | NA |
| 820 | ++ | ++ | NA | ++ | + | NA | NA | NA | NA |
| 821 | +++ | +++ | +++ | +++ | ++ | NA | NA | NA | NA |
| 822 | ++ | +++ | NA | ++ | + | NA | NA | NA | NA |
| 823 | +++ | +++ | NA | +++ | +++ | NA | NA | NA | NA |
| 824 | ++ | ++ | NA | ++ | + | NA | NA | NA | NA |
| 825 | ++ | +++ | NA | ++ | + | NA | NA | NA | NA |
| 826 | +++ | +++ | NA | +++ | ++ | NA | NA | NA | NA |
| 827 | +++ | +++ | +++ | +++ | +++ | NA | NA | NA | NA |
| 828 | + | + | NA | + | + | NA | NA | NA | NA |
| 829 | + | + | NA | + | + | NA | NA | NA | NA |
| 830 | +++ | +++ | NA | +++ | ++ | NA | NA | NA | NA |
| 831 | +++ | +++ | +++ | +++ | ++ | +++ | +++ | +++ | +++ |
| 186a | +++ | +++ | NA | +++ | ++ | +++ | NA | NA | NA |
| 186b | +++ | +++ | NA | ++ | ++ | +++ | NA | NA | NA |
| 187a | ++ | ++ | NA | ++ | + | +++ | NA | NA | NA |
| 187b | +++ | +++ | NA | ++ | ++ | +++ | NA | NA | NA |
| 189a | +++ | +++ | +++ | +++ | ++ | NA | NA | NA | NA |
| 189b | +++ | +++ | NA | +++ | ++ | NA | NA | NA | NA |
| 310a | +++ | +++ | +++ | +++ | +++ | NA | NA | NA | NA |
| 310b | +++ | +++ | NA | +++ | +++ | NA | NA | NA | NA |
| 421a | ++ | ++ | ++ | ++ | + | NA | NA | NA | NA |
| 421b | +++ | +++ | +++ | +++ | ++ | +++ | +++ | +++ | +++ |
| 422a | +++ | +++ | +++ | ++ | ++ | NA | NA | +++ | NA |
| 422b | ++ | ++ | NA | ++ | + | NA | NA | NA | NA |
| 422c | ++ | ++ | ++ | ++ | + | NA | NA | NA | NA |
| 422d | +++ | +++ | +++ | ++ | ++ | NA | NA | NA | NA |
| 445a | +++ | +++ | +++ | +++ | ++ | +++ | +++ | +++ | +++ |
| 445b | ++ | ++ | NA | ++ | + | NA | NA | NA | NA |
| 447a | +++ | +++ | NA | +++ | ++ | NA | NA | NA | NA |
| 447b | ++ | ++ | NA | ++ | + | NA | NA | NA | NA |
| 453a | ++ | ++ | ++ | NA | + | NA | NA | NA | |
| 453b | +++ | +++ | +++ | NA | ++ | NA | NA | NA | +++ |
| 463a | ++ | +++ | NA | ++ | + | NA | NA | NA | NA |
| 463b | +++ | +++ | +++ | +++ | ++ | +++ | +++ | +++ | +++ |
| 468a | + | + | ++ | + | + | NA | NA | NA | NA |
| 468b | ++ | ++ | NA | ++ | + | NA | NA | NA | NA |
| 469a | + | + | + | + | + | NA | NA | NA | NA |
| 469b | + | + | + | + | + | NA | NA | NA | NA |

¹“+++” indicates that IC50 < 100 nM;
“++” indicates that 100 nM <= IC50 < 1000 nM;
“+” indicates that IC50 >= 1000 nM;
“NA” indicates that the IC50 data is not available for this compound.

TABLE 7
IC50 Data for EGFR Activity and Inhibitor Activity on EGFR
phosphorylation (pEGFR)¹-continuted

| | | | BaF3 | | | L858R/ |
|---|---|---|---|---|---|---|
| | BaF3 | BaF3 | L858R/ | L858R | Del19 | C797S |
| Com- | L858R | Del19 | C797S | IC50 | IC50 | IC50 |
| pound # | IC50 | IC50 | IC50 | (pEGFR) | (pEGFR) | (pEGFR) |
| 290 | +++ | +++ | NA | NA | NA | NA |
| 409 | +++ | +++ | +++ | +++ | +++ | +++ |
| 480 | +++ | +++ | +++ | +++ | +++ | +++ |
| 481 | +++ | +++ | +++ | +++ | +++ | +++ |
| 482 | +++ | +++ | +++ | +++ | +++ | +++ |
| 483 | +++ | +++ | NA | NA | NA | NA |
| 515 | +++ | +++ | NA | NA | NA | NA |
| 519 | +++ | +++ | +++ | +++ | +++ | +++ |
| 525 | +++ | +++ | +++ | +++ | +++ | +++ |
| 527 | +++ | +++ | +++ | +++ | +++ | +++ |
| 530 | +++ | +++ | +++ | +++ | +++ | +++ |
| 531 | +++ | +++ | NA | NA | NA | NA |
| 551 | +++ | +++ | +++ | +++ | +++ | +++ |
| 553 | +++ | +++ | NA | NA | NA | NA |
| 559 | +++ | +++ | NA | NA | NA | NA |
| 566 | +++ | +++ | +++ | +++ | +++ | +++ |
| 567 | +++ | +++ | NA | NA | NA | NA |
| 568 | +++ | +++ | +++ | +++ | +++ | +++ |
| 569 | +++ | +++ | +++ | +++ | +++ | +++ |
| 570 | +++ | +++ | +++ | +++ | +++ | +++ |
| 571 | +++ | +++ | NA | NA | NA | NA |
| 572 | +++ | +++ | +++ | +++ | +++ | +++ |
| 573 | +++ | +++ | NA | NA | NA | NA |
| 575 | +++ | +++ | +++ | +++ | +++ | +++ |
| 604 | +++ | +++ | +++ | +++ | +++ | +++ |
| 606 | +++ | +++ | +++ | +++ | +++ | +++ |
| 611 | +++ | +++ | NA | NA | NA | NA |
| 651 | NA | NA | +++ | NA | NA | +++ |
| 655 | +++ | +++ | NA | NA | NA | NA |
| 656 | +++ | +++ | NA | NA | NA | NA |
| 657 | +++ | +++ | +++ | NA | NA | +++ |
| 658 | +++ | +++ | +++ | NA | NA | +++ |
| 679 | +++ | +++ | NA | NA | NA | NA |
| 733 | +++ | +++ | +++ | +++ | +++ | +++ |
| 747 | +++ | +++ | +++ | +++ | +++ | +++ |
| 749 | +++ | +++ | +++ | +++ | +++ | +++ |
| 750 | +++ | +++ | +++ | +++ | +++ | +++ |
| 754 | +++ | +++ | NA | NA | NA | NA |
| 766 | +++ | +++ | NA | NA | NA | NA |
| 778 | +++ | +++ | +++ | +++ | +++ | ++ |
| 779 | +++ | +++ | NA | NA | NA | NA |
| 780 | +++ | +++ | NA | NA | NA | NA |
| 787 | +++ | +++ | +++ | +++ | +++ | +++ |
| 788 | +++ | +++ | +++ | +++ | +++ | +++ |
| 794 | +++ | +++ | +++ | +++ | +++ | +++ |
| 809 | +++ | +++ | +++ | +++ | +++ | +++ |
| 810 | +++ | +++ | NA | NA | NA | NA |
| 817 | +++ | +++ | +++ | +++ | +++ | +++ |
| 819 | +++ | +++ | +++ | +++ | +++ | +++ |
| 820 | +++ | +++ | +++ | +++ | +++ | +++ |
| 821 | +++ | +++ | +++ | +++ | +++ | +++ |
| 822 | +++ | +++ | +++ | +++ | +++ | +++ |
| 823 | +++ | +++ | NA | NA | NA | NA |
| 827 | +++ | +++ | +++ | +++ | +++ | +++ |
| 189a | +++ | +++ | NA | NA | NA | NA |
| 310a | +++ | +++ | NA | NA | NA | NA |
| 421b | +++ | +++ | +++ | +++ | +++ | +++ |
| 422a | +++ | +++ | NA | NA | NA | NA |
| 445a | +++ | +++ | +++ | +++ | +++ | +++ |
| 445b | +++ | +++ | +++ | +++ | +++ | +++ |
| 447a | +++ | +++ | +++ | +++ | +++ | +++ |
| 453a | +++ | +++ | NA | NA | NA | NA |
| 453b | +++ | +++ | +++ | NA | NA | +++ |
| 463b | +++ | +++ | +++ | +++ | +++ | +++ |

¹“+++” indicates that IC50 < 100 nM;
“++” indicates that 100 nM <= IC50 < 1000 nM;
“+” indicates that IC50 >= 1000 nM;
“NA” indicates that the IC50 data is not available for this compound.

What is claimed is:

1. A compound of Formula (I): 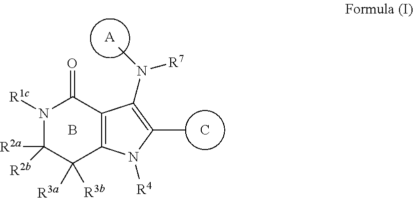
or a pharmaceutically acceptable salt thereof, wherein:
Ring C is selected from the group consisting of: 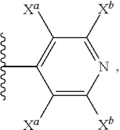
wherein:
each X^{b} is independently X¹, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl), —C(═O)OH; —C(═O)NR′R″; and —SF₅;
2-pyridyl or 3-pyridyl, each optionally substituted with X¹ and further optionally substituted with from 1-4 R^{c};
2-pyridonyl or 4-pyridonyl, each optionally substituted with X¹ and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 substituents independently selected from the group consisting of oxo and R^{c};
C₃₋₁₀ cycloalkyl or C₃₋₁₀ cycloalkenyl, each of which is optionally substituted with X¹ and further optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{c}; and
C₆₋₁₀ aryl optionally substituted with X¹ and further optionally substituted with from 1-4 R^{c};
X¹ is —(X²)ₘ-L¹-R⁵, wherein: m is 0 or 1;
X² is selected from the group consisting of:
—O—, —N(R^{N})—, or —S(O)₀₋₂; 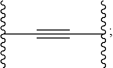 [SMILES: *C#C*]
—C₂₋₆ alkenylene optionally substituted with from 1-3 R^{a};
—C(═O)O—*, —C(═O)N(R^{N})—*, or —S(O)₁₋₂N(R^{N})—*;
—OC(═O)—*, —N(R^{N})C(═O)—*, or —N(R^{N})S(O)₁₋₂—*; and
—OC(═O)N(R^{N})—*, —N(R^{N})C(═O)O—*, —N(R^{N})C(═O)N(R^{N})—*, or —N(R^{N})S(O)₁₋₂N(R^{N})—*,
wherein the asterisk represents point of attachment to L¹;
L¹ is selected from the group consisting of: a bond and C₁₋₁₀ alkylene optionally substituted with from 1-6 R^{a};
R⁵ is selected from the group consisting of:
H;
halo;
—OH;
—NR^{e}R^{f};
—C₁₋₆ alkoxy or —S(O)₀₋₂(C₁₋₆ alkyl), each optionally substituted with from 1-6 R^{a};
—R^{g};
-L⁵-R^{g};
—R^{g2}-R^{W} or —R^{g2}-R^{Y}; and
-L⁵-R^{g2}-R^{W} or -L⁵-R^{g2}-R^{Y};
provided that:
when L¹ is a bond, then R⁵ is selected from the group consisting of: H, —R^{g}, —R^{g2}-R^{W}, and —R^{g2}-R^{Y}; and
X¹ is other than H, —OH, or NH₂;
L⁵ is selected from the group consisting of: —O—, —S(O)₀₋₂, —NH—, and —N(R^{d})—;
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is selected from the group consisting of:
C₂₋₆ alkenyl; C₂₋₆ alkynyl; or C₃₋₁₀ alkenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
bicyclo[x.y.0]cycloalkyl optionally substituted with from 1-2 R^{c}, wherein x is 1 or 2; and y is an integer from 1 to 6;
R^{Y} is selected from the group consisting of: —R^{g} and -(L^{g})_{g}-R^{g};
each of R^{1c}, R^{2a}, R^{2b}, R^{3a}, and R^{3b} is independently selected from the group consisting of; H; halo; —OH, —C(O)OH or —C(O)NH₂; —CN; —R^{b}; -L^{b}-R^{b}; —NR^{e}R^{f}; —R^{g}; -(L^{g})_{g}-R^{g}; -(L^{g})_{g}-R^{W}; -(L^{g})_{g}-R^{g2}-R^{W}; and —C₁₋₆ alkoxy or —C₁₋₆ thioalkoxy, each optionally substituted with from 1-6 R^{a}; provided that R^{1c} is other than halo, —CN, or —C(O)OH; or
two of variables R^{1c}, R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W}; or
one of R^{2a} and R^{2b} and one of R^{3a} and R^{3b} combine to form a double bond between the Ring B atoms to which each is attached;
Ring A is R^{g};
R⁴ and R⁷ are independently H or R^{d};
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of Rh is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl), —C(═O)OH; —C(═O)NR′R″; and —SF₅;
each occurrence of R^{d} is independently selected from the group consisting of: C₁₋₆ alkyl optionally substituted with from 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group;
each occurrence of R′ and R″ is independently selected from the group consisting of: H; —OH; and C₁₋₄ alkyl; and
each occurrence of R^{N} is independently H, C₁₋₃ alkyl, or C₃₋₆ cycloalkyl,
provided that one or more of the following applies:
when R^{2a} and R^{2b} are H or methyl; R^{3a} and R^{3b} are H; Ring C is 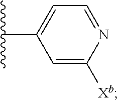
and X^{b} is H, methyl, NH₂, NHC(═O)Me, NHC(═O)iPr, NHC(═O)NHEt, 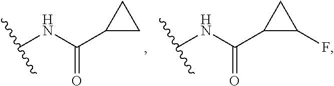
then Ring A is other than unsubstituted phenyl;
when R^{2a}, R^{2b}, R^{3a}, and R^{3b} are each H; Ring C is 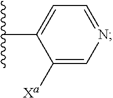
and X^{a} is methyl or F, then Ring A is other than unsubstituted phenyl;
when R^{1C}, R^{2a}, R^{2b}, R^{3a}, and R^{3b} are each H; Ring C is 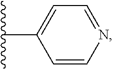 [SMILES: *c1ccncc1]
then Ring A is other than 4-fluorophenyl; and
the compound is other than:

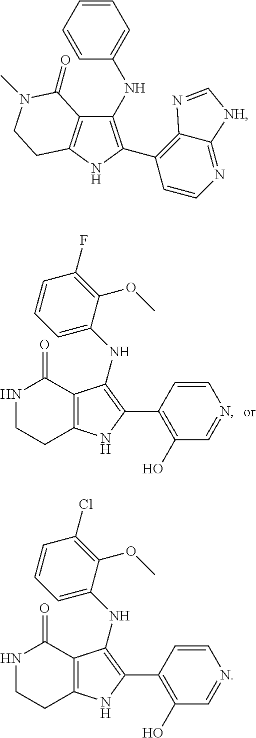 [SMILES: CN1CCc2[nH]c(-c3ccnc4[nH]cnc34)c(Nc3ccccc3)c2C1=O ; COc1c(F)cccc1Nc1c(-c2ccncc2O)[nH]c2c1C(=O)NCC2 ; COc1c(Cl)cccc1Nc1c(-c2ccncc2O)[nH]c2c1C(=O)NCC2]

2. The compound of claim 1, wherein Ring C is heteroaryl including 6 ring atoms, wherein from 2-4 ring atoms are heteroatoms, each independently selected from the group consisting of N, N(H), and N(R^{d}), and wherein the heteroaryl is optionally substituted with X¹ and further optionally substituted with from 1-4 R^{cA}, wherein each R^{cA} is an independently selected R^{c}.

3. The compound of claim 1 or 2, wherein Ring C is heteroaryl including 6 ring atoms, wherein from 2-3 ring atoms are heteroatoms, each independently selected from the group consisting of N, N(H), and N(R^{d}), and wherein the heteroaryl is optionally substituted with from 1-3 R^{cA}, wherein each R^{cA} is an independently selected R^{c}.

4. The compound of any one of claims 1-3, wherein Ring C is pyrimidyl optionally substituted with from 1-3 R^{cA}, such as pyrimidyl substituted with from 1-2 R^{cA}, wherein each R^{cA} is an independently selected R^{c}.

5. The compound of any one of claims 1-4, wherein Ring C is 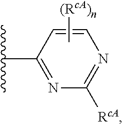
wherein each R^{cA} is an independently selected R^{c}; and n is 0, 1, or 2.

6. The compound of any one of claims 1-5, wherein Ring C is 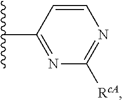
such as

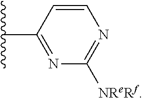

7. The compound of any one of claims 1-5, wherein Ring C is 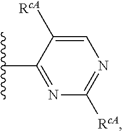
such as

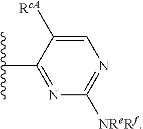

8. The compound of any one of claims 1-6, wherein Ring C is 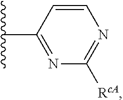
wherein R^{cA} is C₁₋₃ alkyl optionally substituted with from 1-3 independently selected halo.
For example, Ring C is

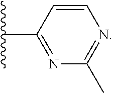 [SMILES: *c1ccnc(C)n1]

9. The compound of any one of claims 1-4, wherein Ring C is

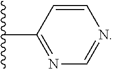 [SMILES: *c1ccncn1]

10. The compound of any one of claims 1-3, wherein Ring C is triazinyl optionally substituted with from 1-2 R^{cA}, wherein each R^{cA} is an independently selected R^{c}, such as wherein Ring C is 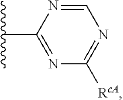
such as

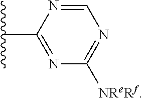

11. The compound of claim 1 or 2, wherein Ring C is heteroaryl including 6 ring atoms, wherein from 2-3 ring atoms are heteroatoms, each independently selected from the group consisting of N, N(H), and N(R^{d}), and wherein the heteroaryl is substituted with X¹ and further optionally substituted with from 1-2 R^{cA}, wherein each R^{cA} is an independently selected R^{c}.

12. The compound of any one of claim 1-2 or 11, wherein Ring C is pyrimidyl substituted with X¹ and further optionally substituted with from 1-2 R^{cA}, wherein each R^{cA} is an independently selected R^{c}.

13. The compound of any one of claim 1-2 or 11-12, wherein Ring C is 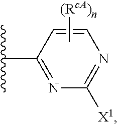
wherein each R^{cA} is an independently selected R^{c}; and n is 0, 1, or 2.

14. The compound of any one of claim 1-2 or 11-13, wherein Ring C is

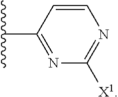

15. The compound of any one of claim 1-2 or 11-12, wherein Ring C is 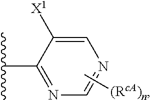
wherein n is 0, 1, or 2; and each R^{1A} is an independently selected R^{c}, such as wherein Ring C is

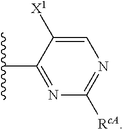

16. The compound of claim 1, wherein Ring C is bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{cA}, wherein each R^{cA} is an independently selected R^{c}.

17. The compound of claim 1 or 16, wherein Ring C is bicyclic heteroaryl including 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{cA}, wherein each R^{cA} is an independently selected R^{c}.

18. The compound of any one of claim 1 or 16-17, wherein Ring C is bicyclic heteroaryl including 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

19. The compound of claim 17 or 18, wherein Ring C is connected to 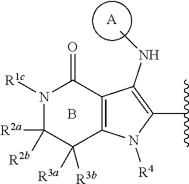
via a 6-membered ring.

20. The compound of any one of claim 1 or 16-19, wherein Ring C is 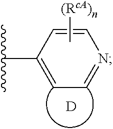
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n is 0, 1, or 2; and each R^{cA} is an independently selected R^{c}.

21. The compound of claim 20, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

22. The compound of any one of claim 1 or 16-21, wherein Ring C is selected from the group consisting of: 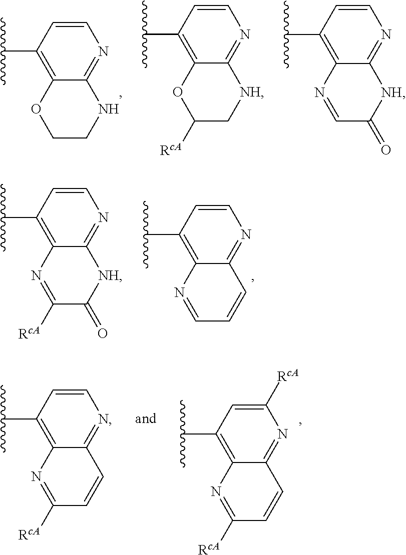
each further optionally substituted with R^{cA}, wherein each R^{cA} is an independently selected R^{c}.

23. The compound of any one of claim 1 or 16-21, wherein Ring C is selected from the group consisting of: 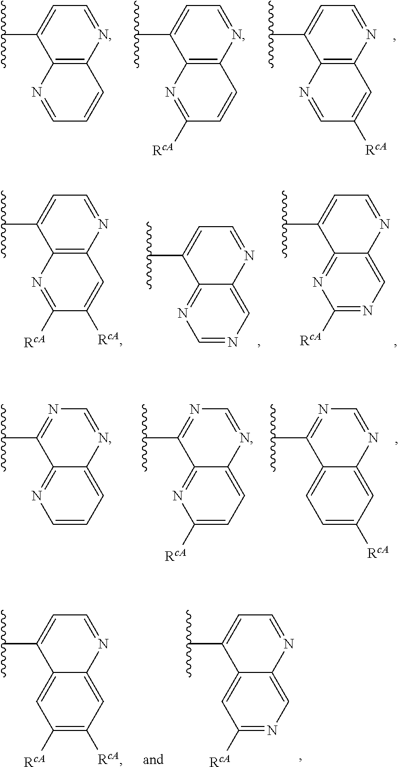
each further optionally substituted with R^{cA}, wherein each R^{cA} is an independently selected R^{c}.

24. The compound of any one of claim 1, 16-21, or claim 23, wherein Ring C is 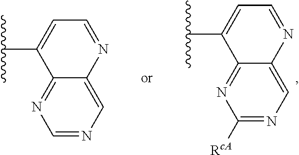
wherein R^{cA} is an independently selected R^{c}.

25. The compound of any one of claim 1, 16-21, or claim 23, wherein Ring C is 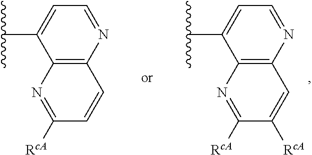
wherein each R^{cA} is an independently selected R^{c}.

26. The compound of any one of claim 1, 16-21, or claim 23, wherein Ring C is selected from the group consisting of: 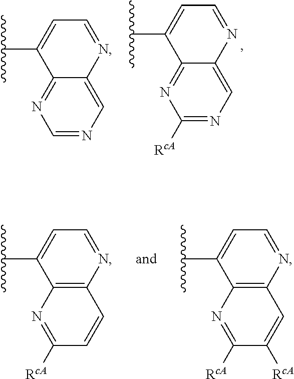
wherein:
each occurrence of R^{cA} is independently selected from the group consisting of: halo; NR^{e}R^{f}; C₁₋₄ alkoxy; C₁₋₄ haloalkoxy: C₁₋₃ alkyl; C₁₋₃ alkyl substituted with from 1-3 independently selected halo; C₁₋₃ alkyl substituted with C₁₋₄ alkoxy; and C₁₋₄ alkoxy substituted with C₁₋₄ alkoxy;
such as wherein each occurrence of R^{cA} is independently selected from the group consisting of: C₁₋₄ alkoxy; C₁₋₄ haloalkoxy; C₁₋₃ alkyl; and C₁₋₃ alkyl substituted with from 1-3 independently selected halo.

27. The compound of claim 20, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

28. The compound of any one of claim 1, 16-20, or 27, wherein Ring C is selected from the group consisting of: 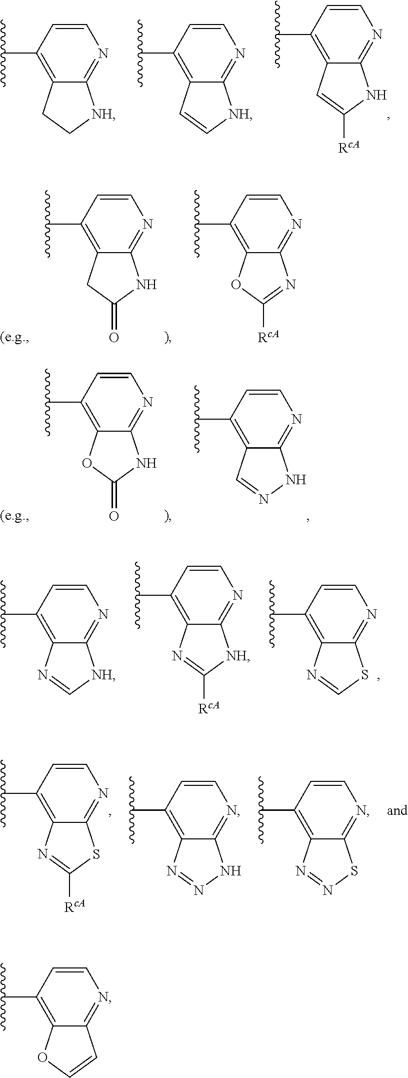
each further optionally substituted with R^{cA}, wherein each R^{cA} is an independently selected R^{c}.

29. The compound of any one of claim 1 or 16-19, wherein Ring C is 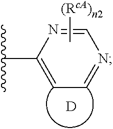
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n2 is 0 or 1; and each R^{cA} is an independently selected R^{c}.

30. The compound of claim 29, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

31. The compound of any one of claim 1, 16-19, or 29-30, wherein Ring C is selected from the group consisting of: 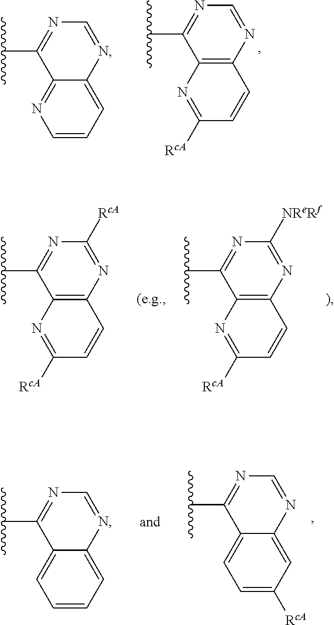
each further optionally substituted with R^{cA}, wherein each R^{cA} is an independently selected R^{c}.

32. The compound of claim 29, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

33. The compound of any one of claim 1, 16-19, 29, or 32, wherein Ring C is selected from the group consisting of: 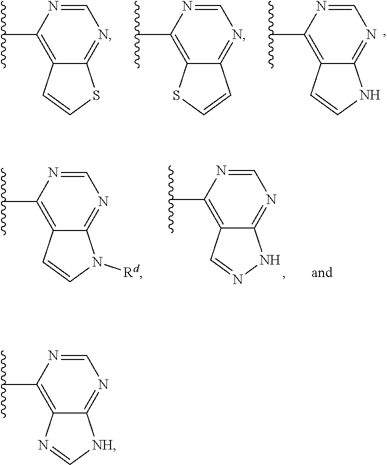
each further optionally substituted with R^{cA}, wherein each R^{cA} is an independently selected R^{c}.

34. The compound of any one of claim 1 or 16-19, wherein Ring C is selected from the group consisting of: 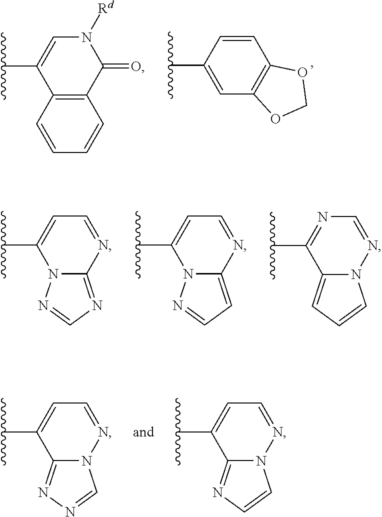
each further optionally substituted with R^{cA}, wherein each R^{cA} is an independently selected R^{c}.

35. The compound of claim 17 or 18, wherein Ring C is connected to 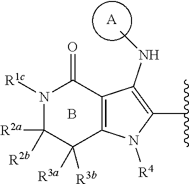
via a 5-membered ring.

36. The compound of any one of claim 1, 16-18, or 35, wherein Ring C is selected from the group consisting of: 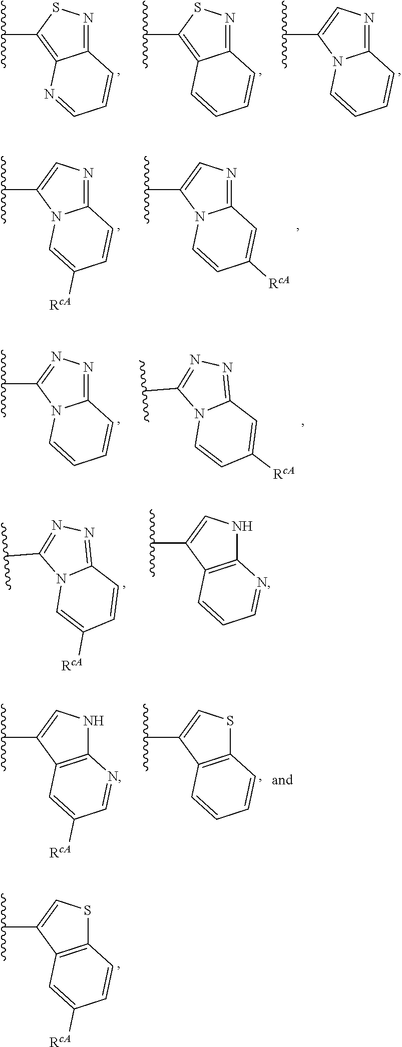
each further optionally substituted with R^{cA}, wherein each R^{cA} is an independently selected R^{c}.

37. The compound of any one of claim 1 or 16-17, wherein Ring C is bicyclic heteroaryl including 9-10 ring atoms, wherein from 1-4 ring atoms are heteroatoms, each independently selected from the group consisting of N, N(H), N(R^{d}), O, and S(O)₀₋₂, and wherein the heteroaryl is substituted with X¹ and further optionally substituted with from 1-4 R^{cA}, wherein each R^{cA} is an independently selected R^{c}.

38. The compound of any one of claim 1, 16-17, 19, or 37, wherein Ring C is 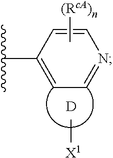
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n is 0, 1, or 2; and each R^{cA} is an independently selected R^{c}.

39. The compound of claim 38, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

40. The compound of any one of claim 1, 16-17, 19, or 37-39, wherein Ring C is selected from the group consisting of: 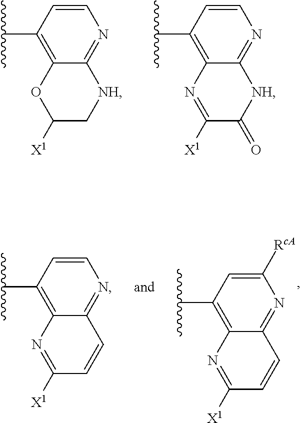
each further optionally substituted with R^{cA}, wherein each R^{cA} is an independently selected R^{c}.

41. The compound of any one of claim 1, 17, or 37, wherein Ring C is selected from the group consisting of: 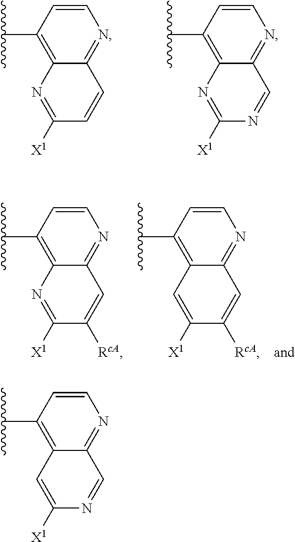
each of which is further optionally substituted with from 1-2 R^{cA}, wherein each R^{cA} is an independently selected R^{c}.

42. The compound of any one of claim 1, 17, 37, or 41, wherein Ring C is

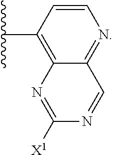

43. The compound of any one of claim 1, 17, 37, or 41, wherein Ring C is

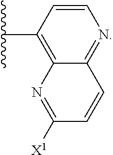

44. The compound of any one of claim 1, 17, 37, or 41, wherein Ring C is 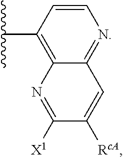
wherein R^{cA} is an independently selected R^{c}.

45. The compound of claim 38, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

46. The compound of any one of claim 1, 16-17, 19, 37-38, or 45, wherein Ring C is selected from the group consisting of: 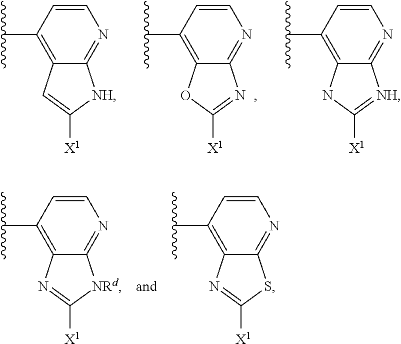
each further optionally substituted with R^{cA}, wherein each R^{cA} is an independently selected R^{c}.

47. The compound of any one of claim 1, 16-17, 19, or 37, wherein Ring C is 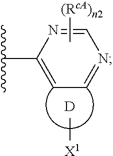
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}; n2 is 0 or 1; and each R^{cA} is an independently selected R^{c}.

48. The compound of claim 47, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

49. The compound of any one of claim 1, 16-17, 19, or 47-48, wherein Ring C is selected from the group consisting of: 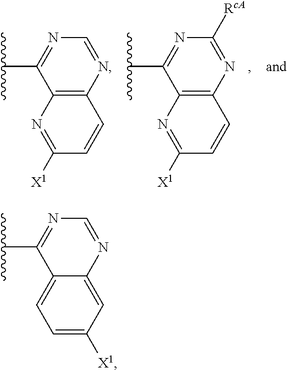
each further optionally substituted with R^{cA}, wherein each R^{cA} is an independently selected R^{c}.

50. The compound of claim 47, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

51. The compound of claim 1, wherein Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X¹ and further optionally substituted with from 1-4 R^{cA}, wherein each R^{cA} is an independently selected R^{c}.

52. The compound of claim 1 or 51, wherein Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{cA}, wherein each R^{cA} is an independently selected R^{c}.

53. The compound of any one of claim 1 or 51-52, wherein Ring C is selected from the group consisting of: pyrazolyl, imidazolyl, thiazolyl, oxazolyl, triazolyl, furanyl, thiophenyl, oxadiazolyl, and thiadiazolyl, each optionally substituted with from 1-2 R^{cA}, wherein a ring nitrogen atom is optionally substituted with R^{d}, and each R^{cA} is an independently selected R^{c}.

54. The compound of any one of claim 1 or 51-53, wherein Ring C is selected from the group consisting of:

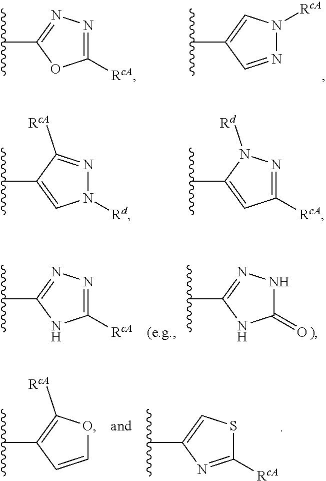

55. The compound of claim 1 or 51, wherein Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is substituted with X¹ and further optionally substituted with from 1-2 R^{cA}, wherein each R^{cA} is an independently selected R^{c}.

56. The compound of any one of claim 1, 51, or 55, wherein Ring C is selected from the group consisting of: pyrazolyl, imidazolyl, thiazolyl, oxazolyl, triazolyl, furanyl, thiophenyl, oxadiazolyl, and thiadiazolyl, each substituted with X¹ and further optionally substituted with from 1-2 R^{cA}, wherein a ring nitrogen atom is optionally substituted with R^{d}, and each R^{cA} is an independently selected R^{c}, such as wherein Ring C is

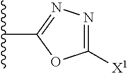

57. The compound of claim 1, wherein Ring C is 2-pyridonyl or 4-pyridonyl, each optionally substituted with X¹ and further optionally substituted with from 1-4 R^{cA}, wherein the ring nitrogen atom is optionally substituted with R^{d}, wherein each R^{cA} is an independently selected R^{c}.

58. The compound of claim 1 or 57, wherein Ring C is 2-pyridonyl which is optionally substituted with X¹ and further optionally substituted with from 1-4 R^{C}A, wherein the ring nitrogen atom is optionally substituted with R^{d}, wherein each R^{cA} is an independently selected R^{c}.

59. The compound of any one of claim 1 or 57-58, wherein Ring C is 2-pyridonyl which is optionally substituted with from 1-4 R^{cA}, wherein the ring nitrogen atom is optionally substituted with R^{d}, wherein each R^{cA} is an independently selected R^{c}, such as wherein Ring C is

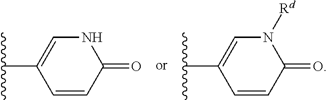

60. The compound of claim 1, wherein Ring C is

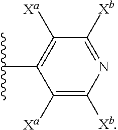

61. The compound of claim 1 or 60, wherein Ring C is

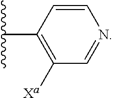

62. The compound of claim 1 or 60, wherein Ring C is 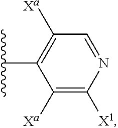
such as

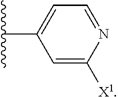

63. The compound of claim 1 or 60, wherein Ring C is 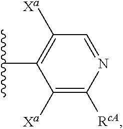
wherein R^{cA} is an independently selected R^{c}, such as wherein Ring C is

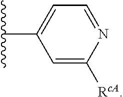

64. The compound of any one of claim 1 or 63, wherein each X¹ is selected from the group consisting of: H; halo; and C₁₋₆ alkyl optionally substituted with from 1-6 R^{a}.

65. The compound of any one of claim 1 or 63-64, wherein from 1-2, such as 1, occurrence of X^{a} is independently a substituent other than H.

66. The compound of any one of claim 1 or 63-64, wherein one occurrence of X^{a} is halo, such as —F or —Cl.

67. The compound of any one of claim 1 or 63-66, wherein one occurrence of X^{a} is —F.

68. The compound of any one of claim 1 or 63-65, wherein one occurrence of X^{a} is C₁₋₃ alkyl optionally substituted with from 1-6 R^{a}.

69. The compound of any one of claim 1 or 63-65, or 68, wherein one occurrence of X^{a} is C₁₋₃ alkyl substituted with from 1-3 independently selected halo, such as —CF₃ or —CHF₂.

70. The compound of any one of claim 1 or 63-64, wherein each X^{a} is —H.

71. The compound of any one of claim 1 or 60-61, wherein Ring C is 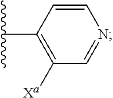
wherein X^{a} is selected from the group consisting of: —F; —Cl; —H; and C₁₋₃ alkyl optionally substituted with from 1-3 independently selected halo.

72. The compound of claim 71, wherein X^{a} is —F.

73. The compound of claim 71, wherein X^{a} is —Cl.

74. The compound of claim 71, wherein X^{a} is —H.

75. The compound of claim 71, wherein X^{a} is C₁₋₃ alkyl substituted with from 1-3 independently selected halo, such as —CF₃ or —CHF₂.

76. The compound of claim 1, wherein Ring C is C₆₋₁₀ aryl optionally substituted with X¹ and further optionally substituted with from 1-4 R^{cA}, wherein each R^{cA} is an independently selected R^{C}.

77. The compound of claim 1 or 76, wherein Ring C is phenyl optionally substituted with from 1-4 R^{cA}, wherein each R^{cA} is an independently selected R^{c}, such as wherein Ring C is 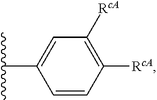
such as

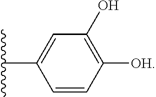 [SMILES: *c1ccc(O)c(O)c1]

78. The compound of claim 1, wherein Ring C is heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X¹ and further optionally substituted with from 1-4 substituents independently selected from the group consisting of oxo and R^{cA}, wherein each R^{cA} is an independently selected R^{c}.

79. The compound of claim 1 or 78, wherein Ring C is heterocyclyl including from 4-8, such as 5-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with X¹ and further optionally substituted with from 1-4 substituents independently selected from the group consisting of oxo and R^{cA}, wherein each R^{cA} is an independently selected R^{c}, such as wherein Ring C is

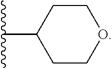 [SMILES: *C1CCOCC1]

80. The compound of any one of claims 1-79, wherein m is 1.

81. The compound of any one of claims 1-80, wherein X² is selected from the group consisting of: —O—, —N(R^{N})—, and —S(O)₀₋₂.

82. The compound of any one of claims 1-81, wherein X² is —N(R^{N})—.

83. The compound of any one of claims 1-82, wherein X² is —N(H)—.

84. The compound of any one of claims 1-81, wherein X² is —O—.

85. The compound of any one of claims 1-80, wherein X² is selected from the group consisting of: —OC(═O)—*, —N(R^{N})C(═O)—*, and —N(R^{N})S(O)₁₋₂—*.

86. The compound of any one of claim 1-80 or 85, wherein X² is —N(R^{N})C(═O)—*.

87. The compound of any one of claim 1-80 or 85-86, wherein X² is —N(H)C(═O)—*.

88. The compound of any one of claim 1-80 or 85, wherein X² is —N(R^{N})S(O)₂—* such as —NHS(O)₂—.

89. The compound of any one of claims 1-57, wherein X² is selected from the group consisting of: —OC(═O)N(R^{N})—*, —N(R^{N})C(═O)O—*, —N(R^{N})C(═O)N(R^{N})—*, and —N(R^{N})S(O)₁₋₂N(R^{N})—*.

90. The compound of any one of claim 1-80 or 89, wherein X² is —N(R^{N})C(═O)O—*, such as —N(H)C(═O)O—*.

91. The compound of any one of claim 1-80 or 89, wherein X² is —N(R^{N})C(═O)N(R^{N})—*, such as —N(H)C(═O)N(H)—*.

92. The compound of any one of claim 1-80 or 89, wherein X² is —C(═O)O—*, —C(═O)N(R^{N})*, or —S(O)₁₋₂N(R^{N})—*.

93. The compound of any one of claim 1-80 or 92, wherein X² is —C(═O)N(R^{N})—*, such as —C(═O)N(H)—*.

94. The compound of any one of claims 1-80, wherein X² is

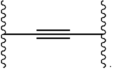 [SMILES: *C#C*]

95. The compound of any one of claims 1-80, wherein X² is C₂₋₆ alkenylene optionally substituted with from 1-3 R^{a}.

96. The compound of any one of claim 1-80 or 95, wherein X² is

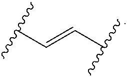 [SMILES: */C=C/*]

97. The compound of any one of claims 1-79, wherein m is 0.

98. The compound of any one of claims 1-97, wherein L¹ is a bond.

99. The compound of any one of claims 1-97, wherein L¹ is C₁₋₁₀ alkylene optionally substituted with from 1-6 R^{a}.

100. The compound of any one of claim 1-97 or 99, wherein L¹ is C₁₋₃ alkylene optionally substituted with from 1-6 R^{a}, such as wherein L¹ is unsubstituted C₁₋₃ alkylene.

101. The compound of any one of claim 1-97 or 99-100, wherein L¹ is —CH₂—, —CH₂CH₂—, —CH₂CF₂—, or —CH(Me)-, such as wherein L¹ is —CH₂—, —CH₂CH₂—, or —CH(Me)-.

102. The compound of any one of claim 1-97 or 99, wherein L¹ is branched C₃₋₆ alkylene optionally substituted with from 1-6 R^{a}, such as wherein L¹ is 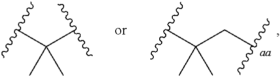
wherein aa is the point of attachment to R⁵.

103. The compound of any one of claim 1-97 or 99-102, wherein R⁵ is —C₁₋₆ alkoxy or —S(O)₀₋₂(C₁₋₆ alkyl), each optionally substituted with from 1-6 R^{a}.

104. The compound of any one of claim 1-97 or 99-103, wherein R⁵ is —C₁₋₆ alkoxy optionally substituted with from 1-6 R^{a}.

105. The compound of any one of claim 1-97 or 99-104, wherein R⁵ is —C₁₋₃ alkoxy, such as methoxy.

106. The compound of any one of claims 1-102, wherein R⁵ is H or halo, such as wherein R⁵ is H or —F.

107. The compound of any one of claim 1-102 or 106, wherein R⁵ is H.

108. The compound of any one of claim 1-97 or 99-102, wherein R⁵ is —OH or —NR^{e}R^{f}.

109. The compound of any one of claim 1-97, 99-102, or 108, wherein R⁵ is —OH.

110. The compound of any one of claims 1-97, wherein R⁵ is —R^{g}.

111. The compound of any one of claim 1-97 or 110, wherein R⁵ is selected from the group consisting of:
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c}.

112. The compound of any one of claim 1-97 or 110-111, wherein R⁵ is C₆₋₁₀ aryl optionally substituted with from 1-4 R^{c}.

113. The compound of any one of claim 1-97 or 110-112, wherein R⁵ is phenyl optionally substituted with from 1-4 R^{c}.

114. The compound of any one of claim 1-102 or 110-113, wherein R⁵ is phenyl optionally substituted with from 1-2 independently selected halo, such as —F.

115. The compound of any one of claim 1-97 or 110-111, wherein R⁵ is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

116. The compound of any one of claim 1-97, 110-111, or 115, wherein R⁵ is heteroaryl including from 5-6 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

117. The compound of any one of claim 1-97, 110-111, or 115-116, wherein R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

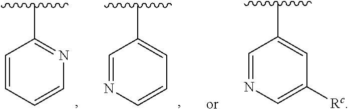

118. The compound of any one of claim 1-97, 110-111, or 115-116, wherein R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

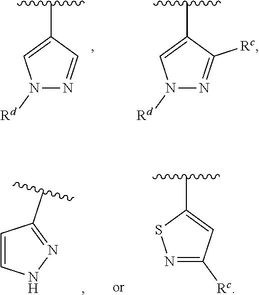

119. The compound of any one of claim 1-97 or 110, wherein R⁵ is selected from the group consisting of:
C₃₋₁₀ cycloalkyl or C₃₋₁₀ cycloalkenyl, each of which is optionally substituted with from 1-4 substituents independently selected from the group consisting of oxo and R^{c}; and
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

120. The compound of any one of claim 1-97, 110, or 119, wherein R⁵ is C₃₋₁₀ cycloalkyl or C₃₋₁₀ cycloalkenyl, each of which is optionally substituted with from 1-4 substituents independently selected from the group consisting of oxo and R^{c}.

121. The compound of any one of claim 1-97, 110, or 119-120, wherein R⁵ is C₃₋₁₀ cycloalkyl, such as C₃₋₆ cycloalkyl, optionally substituted with from 1-4 R^{c}, such as wherein R⁵ is cyclopropyl.

122. The compound of any one of claim 1-97, 110, or 119, wherein R⁵ is heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

123. The compound of any one of claim 1-97, 110, 119, or 122, wherein R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as wherein R⁵ is 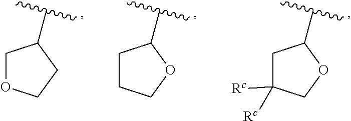
such as

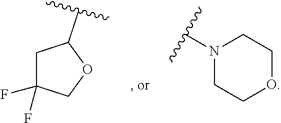 [SMILES: *C1CC(F)(F)CO1 ; *N1CCOCC1]

124. The compound of any one of claims 1-97, wherein R⁵ is selected from the group consisting of: —R^{g2}-R^{W} and —R^{g2}-R^{Y}.

125. The compound of any one of claim 1-97, or 124, wherein R⁵ is —R^{g2}-R^{Y}.

126. The compound of claim 124 or 125, wherein the —R^{g2} group present in R⁵ is C₆₋₁₀ arylene optionally substituted with from 1-4 R^{c}.

127. The compound of any one of claims 124-126, wherein the —R^{g2} group present in R⁵ is phenylene optionally substituted with from 1-4 R^{c}.

128. The compound of any one of claims 124-127, wherein the —R^{g2} group present in R⁵ is 1,3-phenylene or 1,4-phenylene, each optionally substituted with from 1-4 R^{c}, such as wherein —R^{g2} is 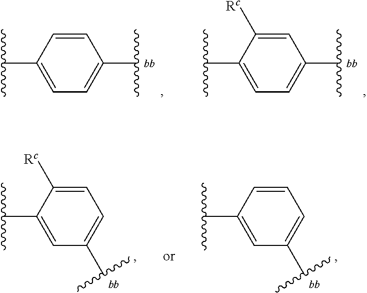
wherein bb is the point of attachment to R^{Y}.

129. The compound of any one of claims 101-105, wherein the R^{Y} group present in R⁵ is —R^{g}.

130. The compound of any one of claims 124-129, wherein the R^{Y} group present in R⁵ is heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c}.

131. The compound of any one of claims 124-130, wherein the R^{Y} group present in R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as wherein R^{Y} is

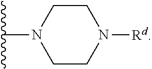

132. The compound of any one of claim 1-97 or 99-102, wherein R⁵ is -L⁵-R^{g}.

133. The compound of any one of claim 1-97, 99-102, or 132, wherein R⁵ is —O—R^{g}.

134. The compound of any one of claim 1-97, 99-102, or 132-133, wherein R⁵ is —O—(C₆₋₁₀ aryl) wherein the C₆₋₁₀ aryl is optionally substituted with from 1-4 R^{c}.

135. The compound of any one of claim 1-97, 99-102, or 132-134, wherein R⁵ is —O-phenyl wherein the phenyl is optionally substituted with from 1-2 R^{c}, such as wherein R⁵ is

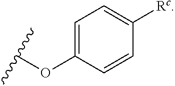

136. The compound of any one of claims 1-79, wherein X¹ is —(X²)ₘ-L¹-R⁵, wherein:
m is 0 or 1;
X² is —N(R^{N})— or —O—;
L¹ is a bond or C₁₋₆ alkylene optionally substituted with from 1-3 R^{a}; and
R⁵ is —R^{g}.

137. The compound of any one of claims 1-79, wherein X¹ is —X²-L¹-R⁵, wherein:
X² is —N(R^{N})C(═O)—*, —N(R^{N})S(O)₂—*, —N(R^{N})C(═O)O—*, or N(R^{N})C(═O)N(R^{N})*;
L¹ is a bond or C₁₋₆ alkylene optionally substituted with from 1-3 R^{a}; and
R⁵ is —R^{g}.

138. The compound of any one of claims 1-79, wherein X¹ is —X²-L¹-R⁵, wherein:
X² is 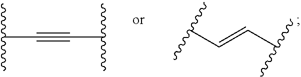
L¹ is a bond or C₁₋₆ alkylene optionally substituted with from 1-3 R^{a}; and
R⁵ is —R^{g}.

139. The compound of any one of claims 136-138, wherein R⁵ is phenyl optionally substituted with from 1-4 R^{c}, such as wherein R⁵ is phenyl optionally substituted with from 1-2 independently selected halo, such as —F.

140. The compound of any one of claims 136-138, wherein R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

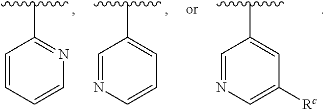

141. The compound of any one of claims 136-138, wherein R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

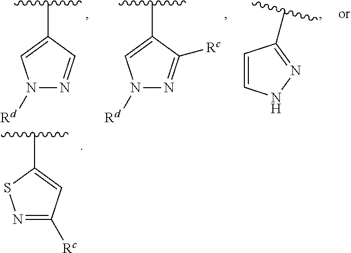

142. The compound of any one of claims 136-138, wherein R⁵ is C₃₋₁₀ cycloalkyl, such as C₃₋₆ cycloalkyl, optionally substituted with from 1-4 R^{c}, such as wherein R⁵ is cyclopropyl.

143. The compound of any one of claims 136-138, wherein R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as wherein R⁵ is 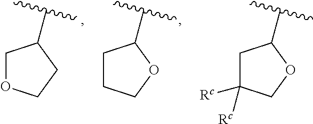
such as

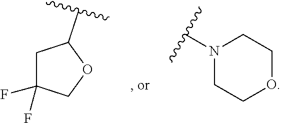 [SMILES: *C1CC(F)(F)CO1 ; *N1CCOCC1]

144. The compound of any one of claims 1-79, wherein X¹ is —(X²)ₘ-L¹-R⁵, wherein:
m is 0 or 1;
X² is —N(R^{N})— or —O—;
L¹ is a bond or C₁₋₆ alkylene optionally substituted with from 1-3 R^{a}; and
R⁵ is —R^{g2}-R^{Y}.

145. The compound of claim 144, wherein the —R^{g2} group present in R⁵ is 1,3-phenylene or 1,4-phenylene, each optionally substituted with from 1-4 R^{c}, such as wherein —R^{g2} is 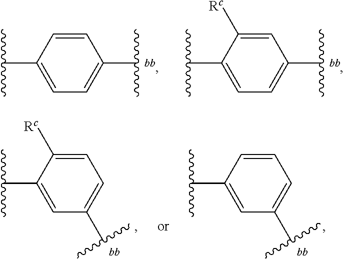
wherein bb is the point of attachment to R^{Y}.

146. The compound of claim 144 or 145, wherein the R^{Y} group present in R⁵ is —R^{g}.

147. The compound of any one of claims 144-146, wherein the R^{Y} group present in R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as wherein R^{Y} is

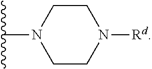

148. The compound of any one of claims 1-79, wherein X¹ is —X²-L¹-R⁵, wherein:
X² is —N(R^{N})—, —O—, —N(R^{N})C(═O)—*, —N(R^{N})S(O)₂—, —N(R^{N})C(═O)O—*, or —N(R^{N})C(═O)N(R^{N})—*;
L¹ is C₁₋₆ alkylene optionally substituted with from 1-3 R^{a}; and
R⁵ is H, halo, C₁₋₆ alkoxy optionally substituted with from 1-3 R^{a}, or —OH.

149. The compound of claim 148, wherein R⁵ is H.

150. The compound of claim 148, wherein R⁵ is halo, such as —F.

151. The compound of claim 148, wherein R⁵ is C₁₋₆ alkoxy optionally substituted with from 1-3 R^{a}, such as wherein R⁵ is C₁₋₃ alkoxy such as methoxy.

152. The compound of claim 148, wherein R⁵ is —OH.

153. The compound of any one of claim 136 or 139-147, wherein m is 0.

154. The compound of any one of claim 136 or 139-147, wherein m is 1.

155. The compound of any one of claim 136, 139-152, or 154, wherein X² is —N(R^{N})—, such as N(H).

156. The compound of any one of claim 136, 139-152, or 154, wherein X² is —O—.

157. The compound of any one of claim 137, 139-143, or 148-152, wherein X² is —N(R^{N})C(═O)—*, such as —N(H)C(═O)—*.

158. The compound of any one of claim 137, 139-143, or 148-152, wherein X² is —N(R^{N})S(O)₂—, such as —N(H)S(O)₂—*.

159. The compound of any one of claim 137, 139-143, or 148-152, wherein X² is —N(R^{N})C(═O)O—*, or —N(R^{N})C(═O)N(R^{N})—*, such as —N(H)C(═O)O—* or —N(H)C(═O)N(H)—*.

160. The compound of any one of claims 138-143, wherein X² is

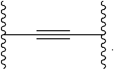 [SMILES: *C#C*]

161. The compound of any one of claims 138-143, wherein X² is

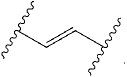 [SMILES: */C=C/*]

162. The compound of any one of claim 136-147 or 153-161, wherein L¹ is a bond.

163. The compound of any one of claims 136-161, wherein L¹ is C₁₋₃ alkylene, such as —CH₂—, —CH₂CH₂—, or —CH(Me)-.

164. The compound of any one of claims 136-161, wherein L¹ is branched C₃₋₆ alkylene, such as 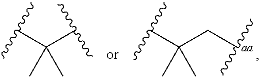
wherein aa is the point of attachment to R⁵.

165. The compound of any one of claims 1-79, wherein X¹ is -L¹-R⁵, wherein L¹ is C₁₋₆ alkylene optionally substituted with from 1-3 R^{a}; and R⁵ is -L⁵-R^{g}.

166. The compound of claim 165, wherein R⁵ is —O—R^{g}.

167. The compound of claim 165 or 166, wherein R⁵ is —O-(phenyl), wherein the phenyl is optionally substituted with from 1-2 R^{c}.

168. The compound of any one of claims 165-167, wherein L¹ is C₁₋₃ alkylene, such as —CH₂—, —CH₂CH₂—, or —CH(Me)-.

169. The compound of any one of claims 2-168, wherein each occurrence of R^{cA} is independently selected from the group consisting of: halo; cyano; C₁₋₁₀ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —NR^{e}R^{f}; —OH; —S(O)₁₋₂NR′R″; —C₁₋₄ thioalkoxy; —C(═O)(C₁₋₁₀ alkyl); —C(═O)O(C₁₋₄ alkyl); —C(═O)OH; and —C(═O)NR′R″.

170. The compound of any one of claims 2-169, wherein one occurrence of R^{cA} is —NR^{e}R^{f}.

171. The compound of any one of claims 2-170, wherein one occurrence of R^{cA} is —NH₂.

172. The compound of any one of claims 2-170, wherein one occurrence of R^{cA} is —NH(C₁₋₆ alkyl), wherein the C₁₋₆ alkyl is optionally substituted with from 1-3 substituents each independently selected from the group consisting of NR′R″, —OH, C₁₋₆ alkoxy, C₁₋₆ haloalkoxy, and halo.

173. The compound of any one of claim 2-170 or 172, wherein one occurrence of R^{cA} is —NHMe, —NHCH₂CF₃, —NHCH₂CH₂OH, or -NHiPr.

174. The compound of any one of claims 2-170, wherein one occurrence of R^{cA} is —NHC(═O)C₁₋₄ alkyl, such as NHC(═O)CH₃; or wherein one occurrence of R^{cA} is N(C₁₋₃ alkyl)₂ such as NMe₂.

175. The compound of any one of claims 2-169, wherein one occurrence of R^{cA} is C₁₋₄ alkoxy optionally substituted with C₁₋₄ alkoxy or C₁₋₄ haloalkoxy, such as wherein one occurrence of R^{cA} is OMe or OCH₂CH₂OMe.

176. The compound of any one of claims 2-169, wherein one occurrence of R^{cA} is C₁₋₄ haloalkoxy, such as —OCH₂CF₃.

177. The compound of any one of claims 2-169, wherein one occurrence of R^{cA} is C₁₋₄ thioalkoxy, such as —SCH₃.

178. The compound of any one of claims 2-169, wherein one occurrence of R^{cA} is C₁₋₆ alkyl, such as methyl; or wherein one occurrence of R^{cA} is C₁₋₆ alkyl substituted with from 1-6 independently selected halo, such as —CF₃.

179. The compound of any one of claims 2-169, wherein one occurrence of R^{cA} is C₁₋₆ alkyl substituted with R^{a}, such as C₁₋₆ alkyl substituted with C₁₋₃ alkoxy or C(═O)NR′R″, such as wherein one occurrence of R^{cA} is

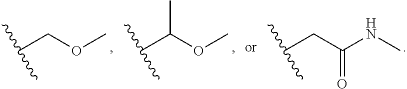

180. The compound of any one of claims 2-169, wherein one occurrence of R^{cA} is halo, such as —F.

181. The compound of any one of claims 2-169, wherein one occurrence of R^{cA} is —OH.

182. The compound of any one of claims 2-169, wherein one occurrence of R^{cA} is C(═O)NR′R″, such as C(═O)NHMe.

183. The compound of any one of claims 1-182, wherein R^{1c} is H.

184. The compound of any one of claims 1-183, wherein R^{2a} and R^{2b} are both H.

185. The compound of any one of claims 1-183, wherein from 1-2 of R^{2a} and R^{2b} is an independently selected substituent that is other than H.

186. The compound of any one of claim 1-183 or 185, wherein one of R^{2a} and R^{2b}, such as R^{2a}, is a substituent that is other than H.

187. The compound of any one of claim 1-183 or 185-186, wherein one of R^{2a} and R^{2b}, such as R^{2a}, is R^{b}.

188. The compound of any one of claim 1-183 or 185-187, wherein one of R^{2a} and R^{2b}, such as R^{2a}, is C₁₋₆ alkyl, which is optionally substituted with from 1-6 R^{a}.

189. The compound of any one of claim 1-183 or 185-188, wherein one of R^{2a} and R^{2b}, such as R^{2a}, is C₁₋₃ alkyl, such as methyl or ethyl.

190. The compound of any one of claims 186-189, wherein the other of R^{2a} and R^{2b}, such as R^{2b}, is H.

191. The compound of any one of claims 1-190, wherein R^{3a} and R^{3b} are both H.

192. The compound of any one of claims 1-190, wherein from 1-2 of R^{3a} and R^{3b} is an independently selected substituent that is other than H.

193. The compound of any one of claim 1-190 or 192, wherein one of R^{3a} and R^{3b}, such as R^{3a}, is a substituent that is other than H.

194. The compound of any one of claim 1-190 or 192-193, wherein one of R^{3a} and R^{3b}, such as R^{3a}, is R^{b}.

195. The compound of any one of claim 1-190 or 192-194, wherein one of R^{3a} and R^{3b}, such as R^{3a}, is C₁₋₆ alkyl which is optionally substituted with from 1-6 R^{a}.

196. The compound of any one of claim 1-190 or 192-194, wherein one of R^{3a} and R^{3b}, such as R^{3a}, is C₁₋₃ alkyl, such as methyl or ethyl.

197. The compound of any one of claim 1-190 or 192-195, wherein one of R^{3a} and R^{3b}, such as R^{3a}, is C₁₋₃ alkyl substituted with from 1-3 independently selected halo.

198. The compound of any one of claim 1-190, 192-195, or 197, wherein one of R^{3a} and R^{3b}, such as R^{3a}, is —CH₂F, —CHF₂, —CF₃, —CH₂CHF₂, or —CH₂CH₂F.

199. The compound of any one of claim 1-190 or 192-195, wherein one of R^{3a} and R^{3b}, such as R^{3a}, is C₁₋₃ alkyl substituted with C₁₋₄ alkoxy, C₁₋₄ haloalkoxy, or NR^{e}R^{f}.

200. The compound of any one of claim 1-190, 192-195, or 199, wherein one of R^{3a} and R^{3b}, such as R^{3a}, is —CH₂OMe, —CH₂CH₂OMe, —CH(Me)CH₂OMe, —CH₂CH(Me)OMe, -CH₂OEt, —CH₂CH₂OCHF₂—CH₂NR^{e}R^{f} (e.g., —CH₂N(CF₃)Me), or —CH₂CH₂NR′Rr (e.g., —CH₂CH₂NMe₂).

201. The compound of any one of claim 1-190, 192-195, or 199-200, wherein one of R^{3a} and R^{3b}, such as R^{3a}, is C₁₋₃ alkyl substituted with C₁₋₄ alkoxy.

202. The compound of any one of claim 1-190, 192-195, or 199-201 wherein one of R^{3a} and R^{3b}, such as R^{3a}, is —CH₂OMe, —CH₂CH₂OMe, —CH(Me)CH₂OMe, —CH₂CH(Me)OMe, or -CH₂OEt, such as —CH₂OMe, such as —CH₂CH₂OMe; optionally the other one of R^{3a} and R^{3b}, such as R^{3b} is H.

203. The compound of any one of claim 1-190 or 192-193, wherein one of R^{3a} and R^{3b}, such as R^{3a}, is R^{g} or -(L^{g})_{g}-R^{g}.

204. The compound of any one of claim 1-190, 192-193, or 203, wherein one of R^{3a} and R^{3b}, such as R^{3a}, is selected from the group consisting of:
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
C₃₋₆ cycloalkyl optionally substituted with from 1-4 R^{c}.

205. The compound of any one of claim 1-190, 192-193, or 203-204, wherein one of R^{3a} and R^{3b}, such as R^{3a}, is selected from the group consisting of: cyclopropyl, cyclobutyl, oxetanyl, and azetidinyl, each of which is optionally substituted with from 1-2 substituents independently selected from the group consisting of: C₁₋₃ alkyl and halo, wherein the ring nitrogen of the azetidinyl is optionally substituted with R^{d}.

206. The compound of any one of claim 1-190, 192-193, or 203, wherein one of R^{3a} and R^{3b}, such as R^{3a}, is —(C₁₋₃ alkylene)-R^{g} or —(C₁₋₃ alkylene)-O—R^{g}, and optionally the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

207. The compound of any one of claim 1-190, 192-193, 203, or 206, wherein one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

208. The compound of any one of claim 1-190, 192-193, 203, or 206-207, wherein one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is selected from the group consisting of:
cyclopropyl, cyclobutyl, oxetanyl, 1,4-dioxanyl, and azetidinyl, each of which is optionally substituted with from 1-2 substituents independently selected from the group consisting of: C₁₋₃ alkyl and halo, wherein the ring nitrogen of the azetidinyl is optionally substituted with R^{d}.

209. The compound of any one of claim 1-190, 192-193, 203 or 206-208, wherein one of R^{3a} and R^{3b}, such as R^{3a}, is selected from the group consisting of: 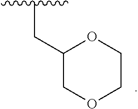 [SMILES: *CC1COCCO1]
such as 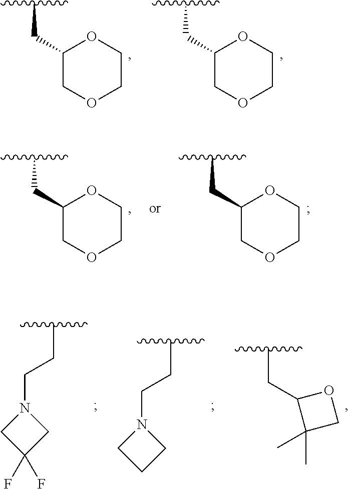
such as 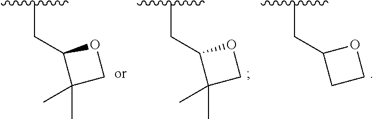
such as 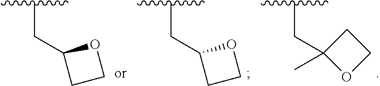
such as

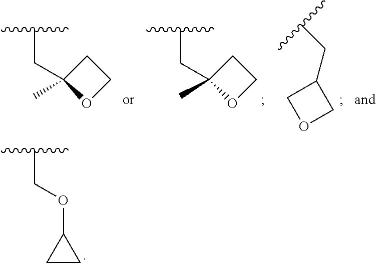

210. The compound of any one of claims 1-190, 192-193 wherein one of R^{3a} and R^{3b}, such as R^{3a}, is -(L^{g})_{g}-R^{W}.

211. The compound any one of claim 1-190, 192-193 or 210, wherein one of R^{3a} and R^{3b}, such as R^{3a}, is —(C₁₋₃ alkylene)-R^{W}; optionally one of R^{3a} and R^{3b}, such as R^{3a}, is —CH₂—R^{W}, or —CH₂CH₂—R^{W}.

212. The compound of claim 1-190, 192-193 or 210-211, wherein the R^{W} group of R^{3a} or R^{3b} is: C(═O)—CH═CH₂, or —NHC(═O)—CH═CH₂.

213. The compound of any claim of 1-190, 192-193 or 210-212, wherein one of R^{3a} and R^{3b}, such as R^{3a}, is 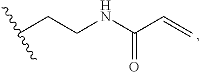 [SMILES: *CCNC(=O)C=C]
such as

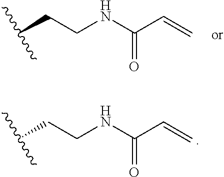

214. The compound of any one of claim 1-190 or 192-193, wherein one of R^{3a} and R^{3b}, such as R^{3a}, is -(L^{g})_{g}-R^{g}—R^{W}.

215. The compound of any one of claim 1-190, 192-193 or 214, wherein one of R^{3a} and R^{3b}, such as R^{3a}, is —(C₁₋₃ alkylene)-R^{g2}-R^{W}, and optionally one of R^{3a} and R^{3b}, such as R^{3a}, is —CH₂—R^{g2}-R^{W}, or —CH₂CH₂—R^{g2}-R^{W}.

216. The compound of any one of claim 1-190, 192-193 or 214-215, wherein the R^{g2} group of R^{3a} or R^{3b} is 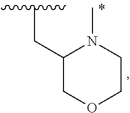 [SMILES: *CC1COCCN1C]
such as 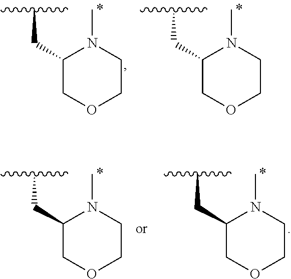
wherein the waveline represents the point of attachment to L^{g} (e.g., —CH₂— or —CH₂CH₂—) and the asterisk represents the point of attachment to R^{W}; and wherein the R^{W} group of R^{3a} or R^{3b} is —C(═O)—CH═CH₂, or —NHC(═O)—CH═CH₂.

217. The compound of any one of claim 1-190, 192-193 or 214-216, wherein one of R^{3a} and R^{3b}, such as R^{3a}, is —CH₂—R^{g2}-R^{W}, and wherein the R^{g2} group of R^{3a} or R^{3b} is 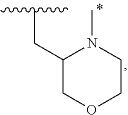 [SMILES: *CC1COCCN1C]
such as 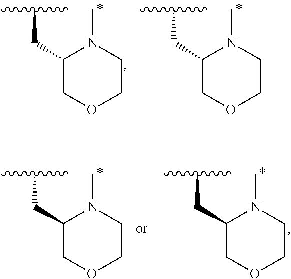
wherein the waveline represents the point of attachment to L^{g} (e.g., —CH₂— or —CH₂CH₂—) and the asterisk represents the point of attachment to R^{W}; and wherein the R^{W} group of R^{3a} or R^{3b} is —C(═O)—CH═CH₂, or —NHC(═O)—CH═CH₂.

218. The compound of any one of claim 1-190, 192-193 or 214-217, wherein one of R^{3a} and R^{3b}, such as R^{3a}, is 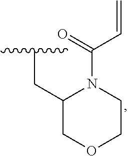 [SMILES: *CC1COCCN1C(=O)C=C]
such as

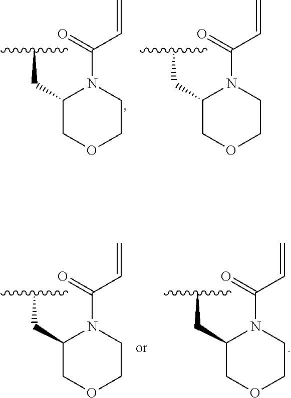

219. The compound of any one of claim 1-190 or 193-218 wherein the other of R^{3a} and R^{3b} is —H.

220. The compound of any one of claim 1-190 or 193-218, wherein the other of R^{3a} and R^{3b} is C₁₋₃ alkyl, such as methyl; or wherein the other of R^{3a} and R^{3b} is halo, such as —F.

221. The compound of any one of claims 1-190, wherein R^{3a} and R^{3b}, together with the Ring B ring atom to which each is attached, form a fused saturated or unsaturated ring of 3-12 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W}.

222. The compound of any one of claim 1-190 or 221, wherein R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

223. The compound of any one of claim 1-190 or 221-222, wherein R^{3a} and R^{3b}, together with the Ring B ring atom to which each is attached, form: 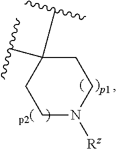
which is optionally substituted with from 1-2 substituents independently selected from the group consisting of oxo and R^{c}, wherein:
p1 and p2 are independently 0, 1, or 2;
R^{Z} is H, R^{d}, C(═O)—W, or S(O)₂W; and
cc represents the point of attachment to C(R^{2a}R^{2b}).

224. The compound of any one of claim 1-190 or 221-223, wherein R^{3a} and R^{3b}, together with the Ring B ring atom to which each is attached, form 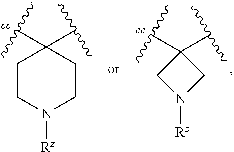
wherein R^{Z} is H, R^{d}, C(═O)—W, or S(O)₂W; and cc represents the point of attachment to C(R^{2a}R^{2b}).

225. The compound of any one of claim 1-190 or 221-223, wherein R^{3a} and R^{3b}, together with the Ring B ring atom to which each is attached, form a fused ring selected from the group consisting of: 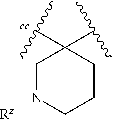
such as 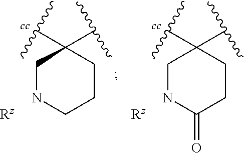
such as 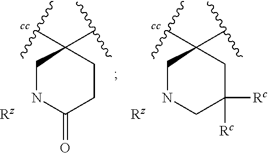
such as 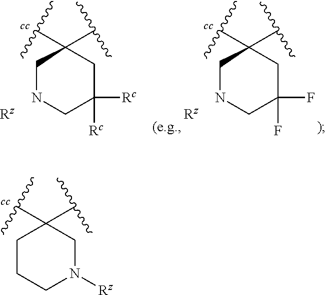
such as 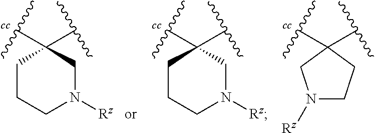
such as 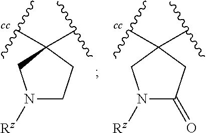
such as 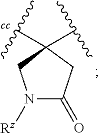
and 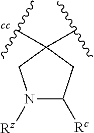
such as 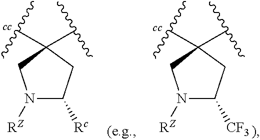
wherein R^{Z} is H, R^{d}, C(═O)—W, or S(O)₂W; and cc represents the point of attachment to C(R^{2a}R^{2b}).

226. The compound of any one of claims 223-225, wherein R^{Z} is H.

227. The compound of any one of claims 223-225, wherein R^{Z} is R^{d}.

228. The compound of any one of claim 223-225 or 227, wherein R^{Z} is C₁₋₆ alkyl optionally substituted with from 1-3 independently selected R^{a}.

229. The compound of any one of claims 223-225, wherein R^{Z} is C(═O)—W or S(O)₂W.

230. The compound of any one of claim 223-225 or 229, wherein W is C₂₋₄ alkenyl.

231. The compound of any one of claim 223-225 or 229-230, wherein R^{Z} is C(═O)—CH₂═CH₂.

232. The compound of any one of claims 1-190, wherein R^{3a} and R^{3b} together with the Ring B ring atom to which each is attached form a fused C₃₋₆ cycloalkyl, wherein the fused C₃₋₆ cycloalkyl is optionally substituted with from 1-2 R^{c}.

233. The compound of any one of claim 1-190 or 232, wherein R^{3a} and R^{3b} together with the Ring B ring atom to which each is attached form a fused cyclopropyl or cyclobutyl.

234. The compound of any one of claims 1-190, wherein R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

235. The compound of any one of claim 1-190 or 234, wherein R^{3a} and R^{3b}, together with the Ring B ring atom to which each is attached, form

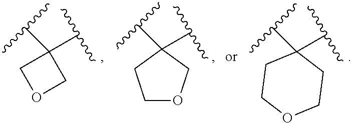 [SMILES: *C1(*)COC1 ; *C1(*)CCOC1 ; *C1(*)CCOCC1]

236. The compound of any one of claims 1-183, wherein one of R^{2a} and R^{2b} (such as R^{2a}) and one of R^{3a} and R^{3b} (such as R^{3a}) taken together with the Ring B ring atoms to which each is attached, form a fused saturated or unsaturated ring of 3-12 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo and R^{c}.

237. The compound of any one of claim 1-183 or 236, wherein one of R^{2a} and R^{2b} (such as R^{2a}) and one of R^{3a} and R^{3b} (such as R^{3a}) taken together with the Ring B ring atoms to which each is attached, form a fused saturated ring of 3-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 3-8 ring atoms is optionally substituted with from 1-4 substituents independently selected from the group consisting of oxo and R^{c}.

238. The compound of any one of claim 1-183 or 236-237, wherein one of R^{2a} and R^{2b} (such as R^{2a}) and one of R^{3a} and R^{3b} (such as R^{3a}) taken together with the Ring B ring atoms to which each is attached, form a fused C34, cycloalkyl which is optionally substituted with from 1-2 R^{c}.

239. The compound of any one of claim 1-183 or 236-238, wherein one of R^{2a} and R^{2b} (such as R^{2a}) and one of R^{3a} and R^{3b} (such as R^{3a}) taken together with the Ring B ring atoms to which each is attached, form a fused cyclopropyl or cyclobutyl.

240. The compound of any one of claims 1-183, wherein one of R^{2a} and R^{2b} (such as R^{2a}) and one of R^{3a} and R^{3b} (such as R^{3a}) combine to form a double bond between the Ring B atoms to which each is attached.

241. The compound of any one of claims 236-240, wherein the other of R^{2a} and R^{2b} and the other of R^{3a} and R^{3b} are each H.

242. The compound of any one of claim 1-183 or 240, wherein the other one of R^{3a} and R^{3b} is R^{g} or -(L^{g})_{g}-R^{g}.

243. The compound of any one of claim 1-183, 240 or 242, wherein the other one of R^{3a} and R^{3b} is -(L^{g})_{g}-R^{g}.

244. The compound of any one of claim 1-183, 240 or 242-243, wherein the other one of R^{3a} and R^{3b} is —(C₁₋₃ alkylene)-R^{g} or —(C₁₋₃ alkylene)-O—R^{g}, and optionally the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

245. The compound of any one of claim 1-183, 240 or 242-244, wherein the other one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

246. The compound of any one of claim 1-183, 240 or 242-245, wherein the other one of R^{3a} and R^{3b}, such as R^{3a}, is —CH₂—R^{g}, —CH₂CH₂R^{g}, or —CH₂—O—R^{g}, wherein the R^{g} group of R^{3a} or R^{3b} is selected from the group consisting of:
cyclopropyl, cyclobutyl, oxetanyl, 1,4-dioxanyl, and azetidinyl, each of which is optionally substituted with from 1-2 substituents independently selected from the group consisting of: C₁₋₃ alkyl and halo, wherein the ring nitrogen of the azetidinyl is optionally substituted with R^{d}.

247. The compound of any one of claim 1-183, 240 or 242-245, wherein the other one of R^{3a} and R^{3b}, such as R^{3a}, is selected from the group consisting of: 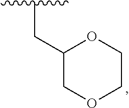 [SMILES: *CC1COCCO1]
such as 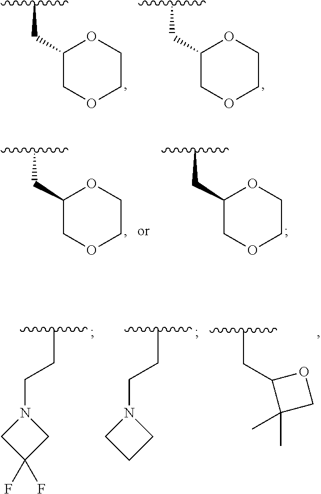
such as 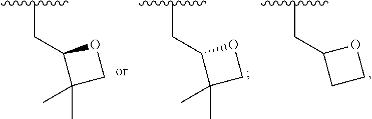
such as 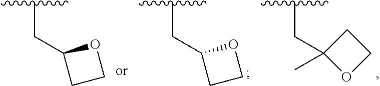
such as

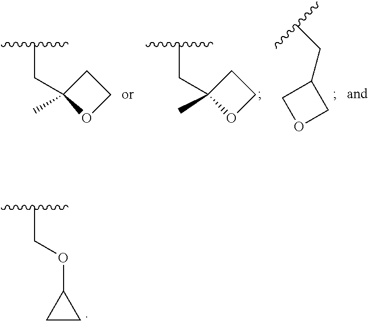

248. The compound of any one of claims 1-183, wherein R^{1c}, R^{2a}, and R^{2b} are each H; one of R^{3a} and R^{3b}, such as R^{3a}, is C₁₋₃ alkyl optionally substituted with from 1-3 R^{a}; and the other of R^{3a} and R^{3b} is H, optionally each R^{a} substituent present in R^{3a} or R^{3b} is independently selected from the group consisting of: halo, C₁₋₄ alkoxy, and C₁₋₄ haloalkoxy.

249. The compound of any one of claims 1-183, wherein R^{1c}, R^{2a}, and R^{2b} are each H; one of R^{3a} and R^{3b}, such as R^{3a}, is C₁₋₃ alkyl optionally substituted with from C₁₋₄ alkoxy; optionally one of R^{3a} and R^{3b}, such as R^{3a}, is —CH₂CH₂—OMe; and the other of R^{3a} and R^{3b} is H.

250. The compound of any one of claims 1-183, wherein R^{1c}, R^{2a}, and R^{2b} are each H; and R^{3a} and R^{3b} are independently selected C₁₋₃ alkyl.

251. The compound of any one of claims 1-183, wherein R^{1c}, R^{2a}, and R^{2b} are each H; one of R^{3a} and R^{3b}, such as R^{3a}, is —R^{g}, —(C₁₋₃ alkylene)-R^{g}, or —(C₁₋₃ alkylene)-O—R^{g},
optionally wherein the R^{g} group of R^{3a} or R^{3b} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, and
the other of R^{3a} and R^{3b} is H.

252. The compound of any one of claims 1-183, wherein R^{1c}, R^{2a}, and R^{2b} are each H; and R^{3a} and R^{3b} taken together with the Ring B ring carbon atom to which each is attached form a fused C₃₋₆ (such as C₃ or C₄) cycloalkyl, wherein the fused cycloalkyl ring is optionally substituted with from 1-2 R^{c}.

253. The compound of any one of claims 1-183, wherein R^{1c}, R^{2a}, and R^{2b} are each H; and R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

254. The compound of any one of claims 1-183, wherein R^{1c} is H; one of R^{2a} and R^{2b} (such as R^{2a}) and one of R^{3a} and R^{3b} (such as R^{3a}) taken together with the Ring B ring atoms to which each is attached, form a fused C₃₋₆ (such as C₃ or C₄) cycloalkyl which is optionally substituted with from 1-2 R^{c}; and the other of R^{2a} and R^{2b} and the other of R^{3a} and R^{3b} are each H.

255. The compound of any one of claims 1-183, wherein R^{1c}, R^{2a}, R^{2b}, R^{3a}, and R^{3b} are each H.

256. The compound of any one of claims 1-238, wherein R⁴ is H; and R⁷ is H.

257. The compound of any one of claims 1-256, wherein Ring A is 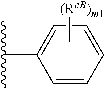
wherein each R^{cB} is an independently selected R^{c}; and m1 is 0, 1, 2, 3, or 4.

258. The compound of claim 257, wherein m1 is 1, 2, or 3.

259. The compound of claim 257 or 258, wherein m1 is 1 or 2, such as 2.

260. The compound of any one of claims 1-259, wherein Ring A is 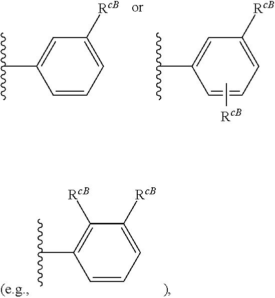
wherein each R^{cB} is an independently selected R^{c}.

261. The compound of any one of claims 1-259, wherein Ring A is selected from the group consisting of: 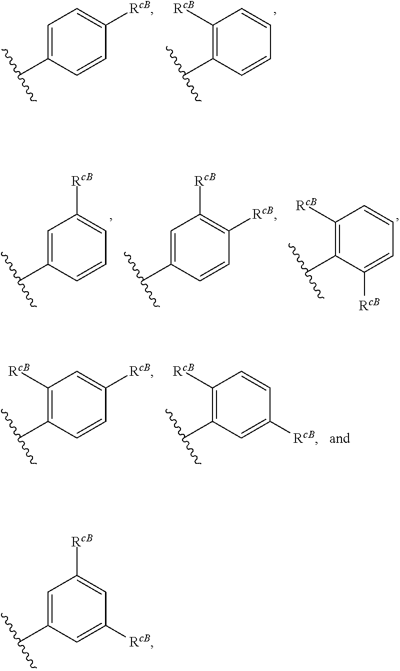
wherein each R^{cB} is an independently selected R^{c}.

262. The compound of any one of claims 257-261, wherein each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

263. The compound of any one of claims 1-256, wherein Ring A is 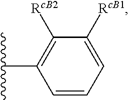
wherein R^{cB1} is R^{c}; and R^{cB2} is H or R^{c}, optionally wherein R^{cB1} and R^{cB2} are each independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

264. The compound of claim 263, wherein R^{cB1} is halo, such as —F or —Cl, such as —F.

265. The compound of claim 263, wherein R^{cB1} is C₁₋₃ alkyl or C₁₋₃ alkyl substituted with from 1-6 independently selected halo, such as wherein R^{cB1} is methyl, —CHF₂, or —CF₃.

266. The compound of any one of claims 263-265, wherein R^{cB2} is selected from the group consisting of: halo; —CN; C₁₋₄ alkoxy; C₁₋₄ haloalkoxy; C₁₋₃ alkyl; and C₁₋₃ alkyl substituted with from 1-6 independently selected halo.

267. The compound of any one of claims 263-266, wherein R^{cB2} is C₁₋₄ alkoxy or C₁₋₄ haloalkoxy.

268. The compound of any one of claims 263-267, wherein R^{cB2} is selected from the group consisting of cyano; C₁₋₃ alkyl; and C₁₋₃ alkyl substituted with from 1-6 independently selected halo, such as wherein R^{cB2} is cyano, methyl, ethyl, —CHF₂, —CF₃, or —CH₂CHF₂.

269. The compound of any one of claim 1-256, or 263-268, wherein Ring A is

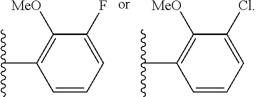

270. The compound of any one of claims 1-256, wherein Ring A is heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

271. The compound of any one of claim 1-256 or 270, wherein Ring A is bicyclic heteroaryl including from 9-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}.

272. The compound of any one of claim 1-256 or 270-271, wherein Ring A is selected from the group consisting of: 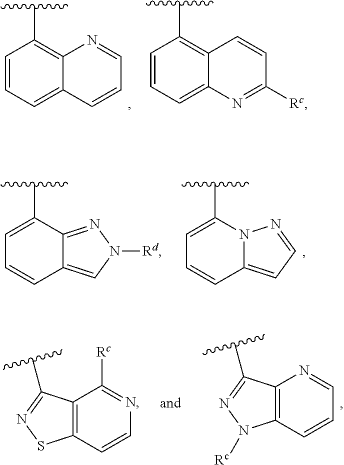
each of which is further optionally substituted with R^{c}.

273. The compound of claim 1, wherein the compound is a compound of Formula (I-a): 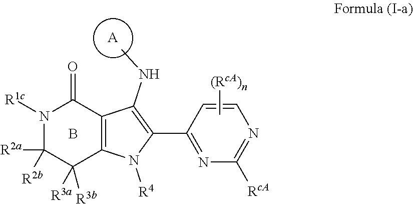
or a pharmaceutically acceptable salt thereof,
wherein: each R^{cA} is an independently selected R^{c}; and n is 0, 1, or 2.

274. The compound of claim 273, wherein 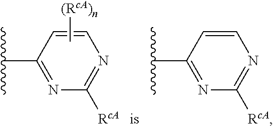
such as

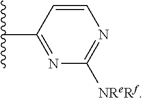

275. The compound of claim 273 or 274, wherein 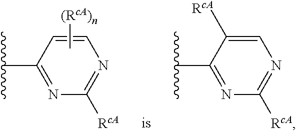
such as

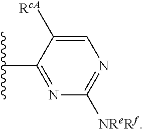

276. The compound of any one of claim 273 or 274, wherein 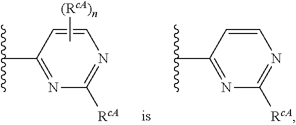
herein R^{cA} is C₁₋₃ alkyl optionally substituted with from 1-3 independently selected halo; optionally wherein

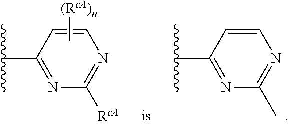

277. The compound of any one of claims 273-276, wherein one of R^{3a} and R^{3b}, such as R^{3a}, is C₁₋₃ alkyl substituted with C₁₋₄ alkoxy; optionally wherein the other one of of R^{3a} and R^{3b}, such as R^{3b} is H.

278. The compound of any one of claim 276 wherein one of R^{3a} and R^{3b}, such as R^{3a}, is —CH₂OMe, —CH₂CH₂OMe, —CH(Me)CH₂OMe, —CH₂CH(Me)OMe, or -CH₂OEt; optionally wherein one of R^{3a} and R^{3b}, such as R^{3a} is —CH₂CH₂OMe.

279. The compound of claim 1, wherein the compound is a compound of Formula (I-b): 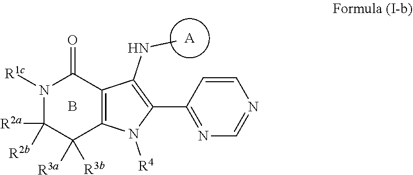
or a pharmaceutically acceptable salt thereof.

280. The compound of claim 1, wherein the compound is a compound of Formula (I-c): 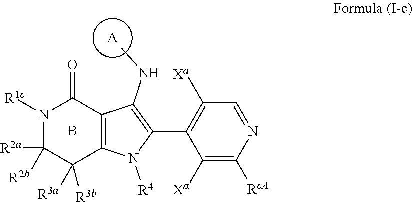
or a pharmaceutically acceptable salt thereof,
wherein: R^{cA} is an independently selected R^{c}.

281. The compound of claim 280, wherein

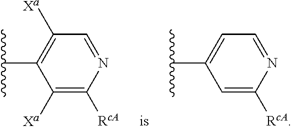

282. The compound of claim 1, wherein the compound is a compound of Formula (I-d): 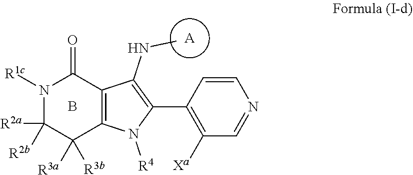
or a pharmaceutically acceptable salt thereof, wherein:
X^{a} is selected from the group consisting of: H; —F; —Cl; C₁₋₆ alkyl; and C₁₋₃ alkyl substituted with from 1-3 independently selected halo.

283. The compound of claim 282, wherein X^{a} is —F.

284. The compound of claim 82, wherein X^{a} is C₁₋₃ substituted with from 1-3 independently selected halo, such as —CF₂H or —CF₃.

285. The compound of claim 1, wherein the compound is a compound of Formula (I-e): 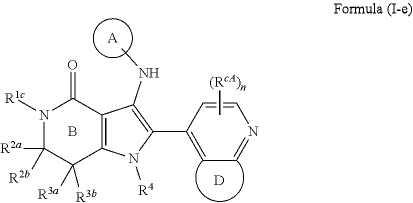
or a pharmaceutically acceptable salt thereof,
wherein:
each R^{cA} is an independently selected R^{c};
n is 0, 1, or 2; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

286. The compound of claim 285, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

287. The compound of claim 285 or 286, wherein 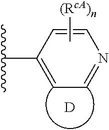
is selected from the group consisting of: 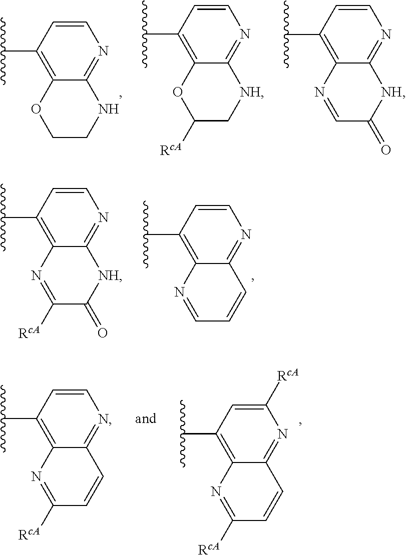
each further optionally substituted with R^{cA}, wherein each R^{cA} is an independently selected R^{c}.

288. The compound of claim 285 or 286, wherein 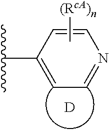
is selected from the group consisting of: 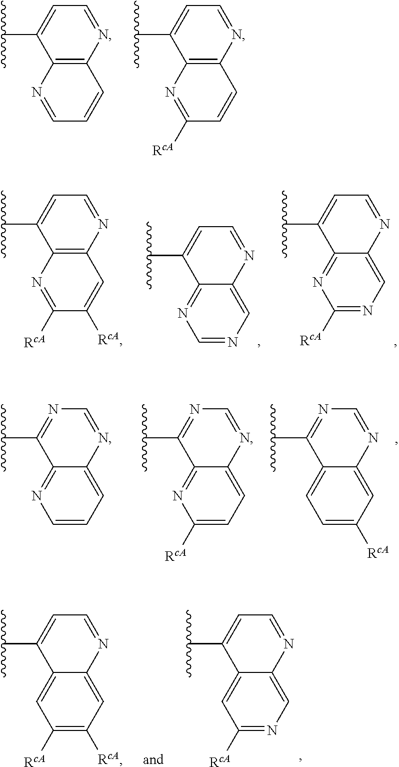
each further optionally substituted with R^{cA}, wherein each R^{cA} is an independently selected R^{c}.

289. The compound of any one of claims 285-288, wherein 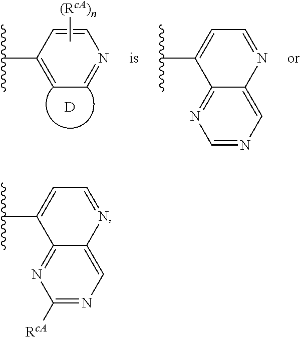
wherein R^{cA} is an independently selected R^{c}.

290. The compound of any one of claims 285-288, wherein 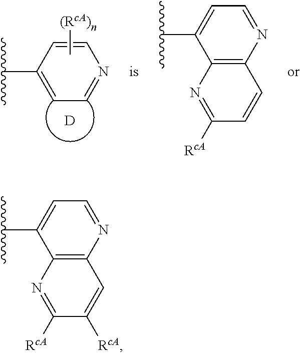
wherein each R^{cA} is an independently selected R^{c}.

291. The compound of any one of claims 285-288, wherein 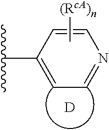
is selected from the group consisting of: 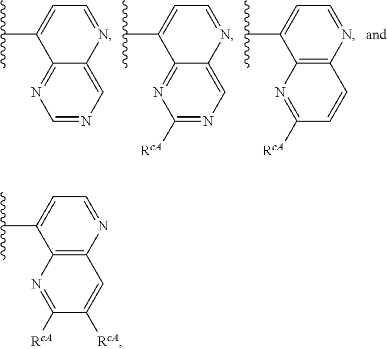
wherein:
each occurrence of R^{cA} is independently selected from the group consisting of: halo; NR^{e}R^{f}; C₁₋₄ alkoxy; C₁₋₄ haloalkoxy; C₁₋₃ alkyl; C₁₋₃ alkyl substituted with from 1-3 independently selected halo; C₁₋₃ alkyl substituted with C₁₋₄ alkoxy; and C₁₋₄ alkoxy substituted with C₁₋₄ alkoxy;
such as wherein each occurrence of R^{cA} is independently selected from the group consisting of: C₁₋₄ alkoxy; C₁₋₄ haloalkoxy; C₁₋₃ alkyl; and C₁₋₃ alkyl substituted with from 1-3 independently selected halo.

292. The compound of claim 285, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

293. The compound of claim 285 or 292, wherein 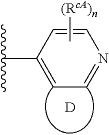
is selected from the group consisting of: 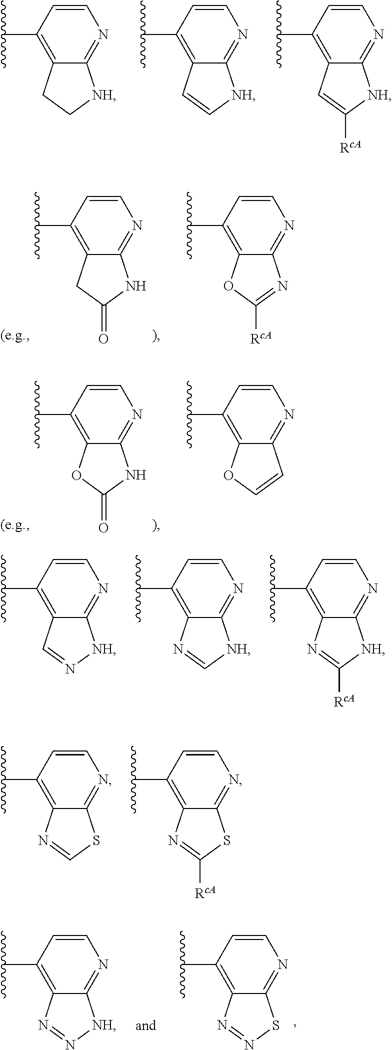
each further optionally substituted with R^{cA}, wherein each R^{cA} is an independently selected R^{c}.

294. The compound of claim 1, wherein the compound is a compound of Formula (I-f): 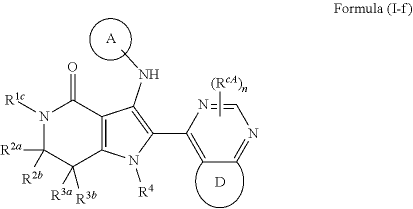
or a pharmaceutically acceptable salt thereof,
wherein:
each R^{cA} is an independently selected R^{c};
n is 0 or 1; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

295. The compound of claim 294, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

296. The compound of claim 294 or 295, wherein 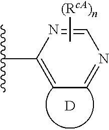
is selected from the group consisting of: 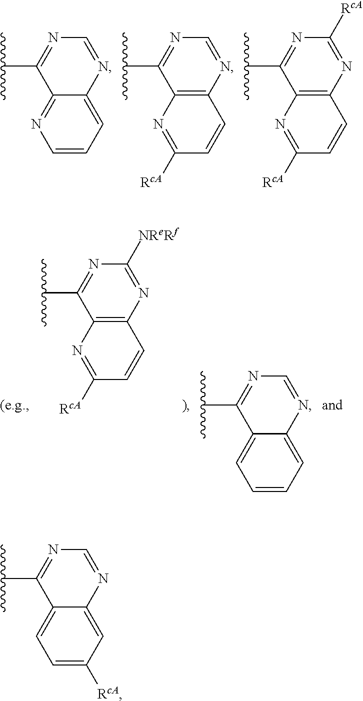
each further optionally substituted with R^{cA}, wherein each R^{cA} is an independently selected R^{c}.

297. The compound of claim 294, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

298. The compound of claim 294 or 297, wherein 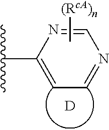
is selected from the group consisting of: 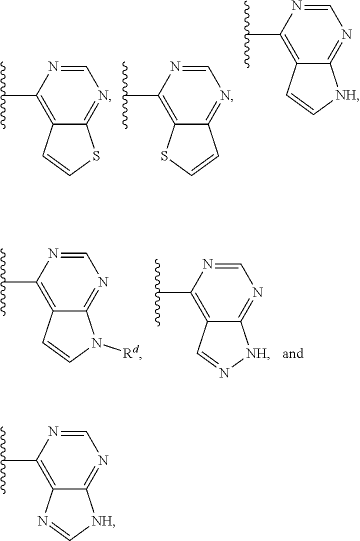
each further optionally substituted with R^{cA}, wherein each R^{a} is an independently selected R^{c}.

299. The compound of claim 1, wherein the compound is a compound of Formula (I-g): 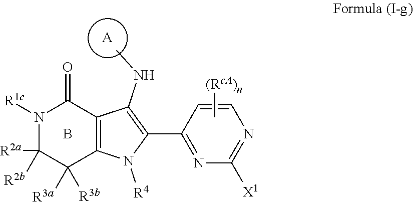
or a pharmaceutically acceptable salt thereof,
wherein: each R^{cA} is an independently selected R^{c}; and n is 0, 1, or 2.

300. The compound of claim 299, wherein

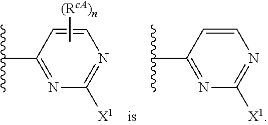

301. The compound of claim 1, wherein the compound is a compound of Formula (I-h): 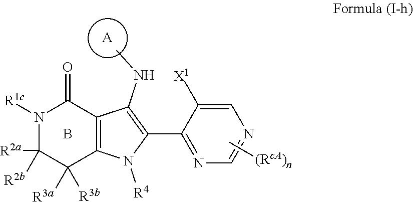
or a pharmaceutically acceptable salt thereof,
wherein: each R^{cA} is an independently selected R^{c}; and n is 0, 1, or 2.

302. The compound of claim 301, wherein 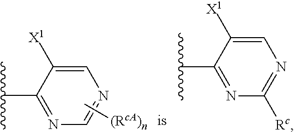
such as

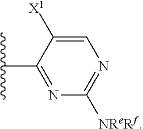

303. The compound of claim 1, wherein the compound is a compound of Formula (I-i): 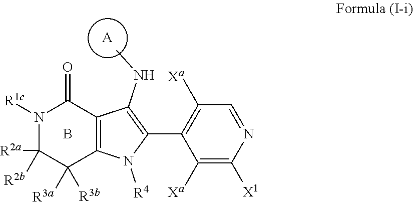
or a pharmaceutically acceptable salt thereof.

304. The compound of claim 303, wherein each X^{a} is H.

305. The compound of claim 1, wherein the compound is a compound of Formula (I-j): 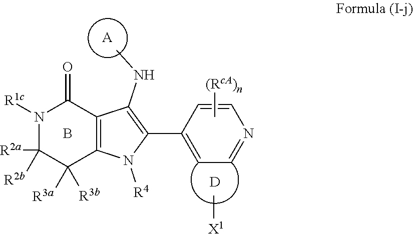
or a pharmaceutically acceptable salt thereof;
wherein n is 0, 1, or 2;
each R^{cA} is an independently selected R^{c}; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

306. The compound of claim 305, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

307. The compound of claim 305 or 306, wherein 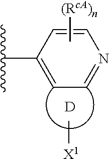
is selected from the group consisting of: 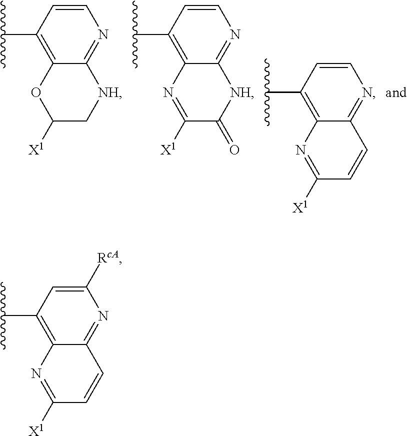
each further optionally substituted with R^{cA}, wherein each R^{cA} is an independently selected R^{c}.

308. The compound of any one of claim 305 or 306, wherein 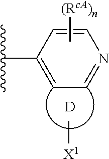
is selected from the group consisting of: consisting of: 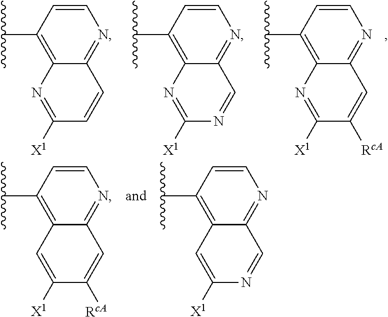
each of which is further optionally substituted with from 1-2 R^{cA}, wherein each R^{cA} is an independently selected R^{c}.

309. The compound of any one of claim 305, 306 or 308, wherein 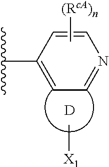
is selected from the group consisting of: consisting of:

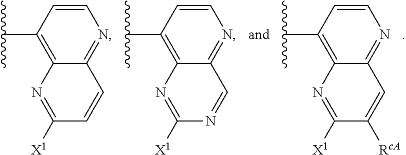

310. The compound of claim 305, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

311. The compound of claim 305 or 310, wherein 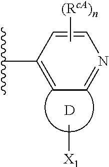
is selected from the group consisting of: 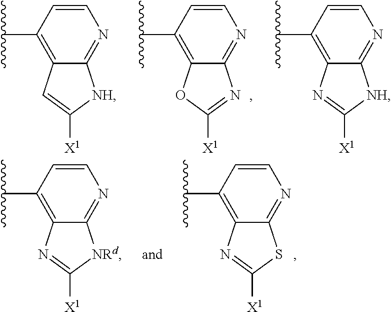
each further optionally substituted with R^{cA}, wherein each R^{cA} is an independently selected R^{c}.

312. The compound of claim 1, wherein the compound is a compound of Formula (I-k): 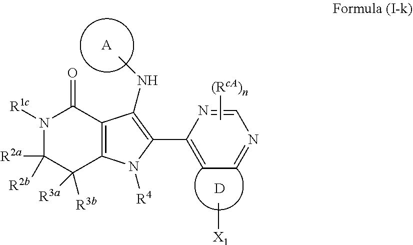
or a pharmaceutically acceptable salt thereof;
wherein n is 0 or 1;
each R^{cA} is an independently selected R^{c}; and
Ring D is a partially unsaturated or aromatic ring including from 5-6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

313. The compound of claim 312, wherein Ring D is a partially unsaturated or aromatic ring including 6 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

314. The compound of claim 312 or 313, wherein 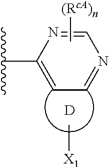
is selected from the group consisting of: 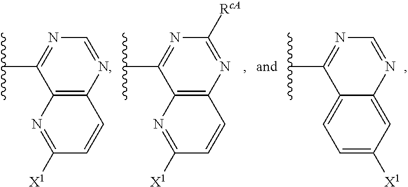
each further optionally substituted with R^{cA}, wherein each R^{cA} is an independently selected R^{c}.

315. The compound of claim 312, wherein Ring D is a partially unsaturated or aromatic ring including 5 ring atoms, wherein from 0-2 of the ring atoms are heteroatoms each independently selected from the group consisting of N, N(H), N(R^{d}), O, and S(O)₀₋₂, wherein Ring D is optionally substituted with from 1-2 R^{cA}.

316. The compound of any one of claims 273-315, wherein each occurrence of R^{cA} is independently selected from the group consisting of: halo; cyano; C₁₋₁₀ alkyl which is optionally substituted with from 1-6 independently selected R^{a}; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —NR^{e}R^{f}; —OH; —S(O)₁₋₂NR′R″; —C₁₋₄ thioalkoxy; —C(═O)(C₁₋₁₀ alkyl); —C(═O)O(C₁₋₄ alkyl); —C(═O)OH; and —C(═O)NR′R″.

317. The compound of any one of claims 273-316, wherein one occurrence of R^{cA} is —NR^{e}R^{f}.

318. The compound of any one of claims 273-317, wherein one occurrence of R^{cA} is —NH₂.

319. The compound of any one of claims 273-317, wherein one occurrence of R^{cA} is —NH(C₁₋₆ alkyl), wherein the C₁₋₆ alkyl is optionally substituted with from 1-3 substituents each independently selected from the group consisting of NR′R″, —OH, C₁₋₆ alkoxy, C₁₋₆ haloalkoxy, and halo, such as wherein one occurrence of R^{cA} is —NHMe, —NHCH₂CF₃, —NHCH₂CH₂OH, or NHiPr.

320. The compound of any one of claims 273-317, wherein one occurrence of R^{cA} is —NHC(═O)C₁₋₄ alkyl, such as NHC(═O)CH₃; or wherein one occurrence of R^{cA} is N(C₁₋₃ alkyl)₂ such as NMe₂.

321. The compound of any one of claims 273-316, wherein one occurrence of R^{cA} is C₁₋₄ alkoxy optionally substituted with C₁₋₄ alkoxy or C₁₋₄ haloalkoxy, such as wherein one occurrence of R^{cA} is OMe or OCH₂CH₂OMe; or wherein one occurrence of R^{cA} is C₁₋₄ haloalkoxy, such as —OCH₂CF₃.

322. The compound of any one of claims 273-316, wherein one occurrence of R^{cA} is C₁₋₄ thioalkoxy, such as —SCH₃.

323. The compound of any one of claims 273-316, wherein one occurrence of R^{cA} is C₁₋₆ alkyl, such as methyl; or wherein one occurrence of R^{cA} is C₁₋₆ alkyl substituted with from 1-6 independently selected halo, such as —CF₃.

324. The compound of any one of claims 273-316, wherein one occurrence of R^{cA} is C₁₋₆ alkyl substituted with R^{a}, such as C₁₋₆ alkyl substituted with C₁₋₃ alkoxy or C(═O)NR′R″, such as wherein one occurrence of R^{cA} is

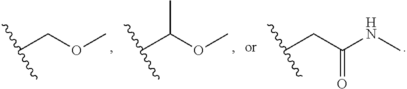

325. The compound of any one of claims 273-316, wherein one occurrence of R^{cA} is halo, such as —F.

326. The compound of any one of claims 273-316, wherein one occurrence of R^{cA} is —OH.

327. The compound of any one of claims 273-316, wherein one occurrence of R^{cA} is C(═O)NR′R″, such as C(═O)NHMe.

328. The compound of any one of claims 299-327, wherein X¹ is —(X²)ₘ-L¹-R⁵, wherein:
m is 0 or 1;
X² is —N(R^{N})— or —O—;
L¹ is a bond or C₁₋₆ alkylene optionally substituted with from 1-3 R^{a}, and
R⁵ is —R^{g}.

329. The compound of any one of claims 299-327, wherein X¹ is —X²-L¹-R⁵, wherein:
X² is —N(R^{N})C(═O)*, —N(R^{N})S(O)₂*, —N(R^{N})C(═O)O—*, or —N(R^{N})C(═O)N(R^{N})—*;
L¹ is a bond or C₁₋₆ alkylene optionally substituted with from 1-3 R^{a}; and
R⁵ is —R^{g}.

330. The compound of any one of 299-327, wherein X¹ is —X²-L¹-R⁵, wherein:
X² is 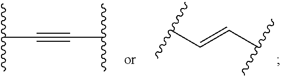
L¹ is a bond or C₁₋₆ alkylene optionally substituted with from 1-3 R^{a}; and
R⁵ is —R^{g}.

331. The compound of any one of claims 328-330, wherein R⁵ is phenyl optionally substituted with from 1-4 R^{c}, such as wherein R⁵ is phenyl optionally substituted with from 1-2 independently selected halo, such as —F.

332. The compound of any one of claims 328-330, wherein R⁵ is heteroaryl including 6 ring atoms, wherein from 1-4 ring atoms are heteroatoms, each independently selected from the group consisting of N, N(H), and N(R^{d}), and wherein the heteroaryl is optionally substituted with from 1-4 R^{c}, such as wherein R⁵ is

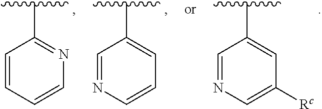

333. The compound of any one of claims 328-330, wherein R⁵ is heteroaryl including 5 ring atoms, wherein from 1-4, such as 2-4, ring atoms are heteroatoms, each independently selected from the group consisting of N, N(H), N(R^{d}), O, and S(O)₀₋₂, and wherein the heteroaryl is optionally substituted with from 1-4 R^{c}, such as wherein R⁵ is

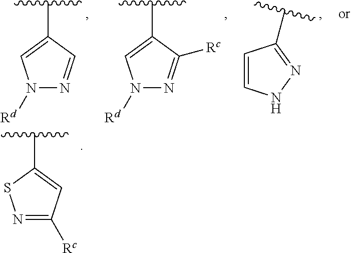

334. The compound of any one of claims 328-330, wherein R⁵ is C₃₋₁₀ cycloalkyl, such as C₃₋₆ cycloalkyl, optionally substituted with from 1-4 R^{c}, such as wherein R⁵ is cyclopropyl.

335. The compound of any one of claims 328-330, wherein R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as wherein R⁵ is 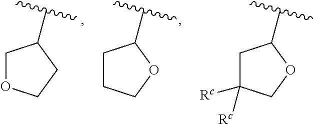
such as

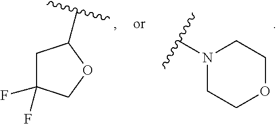 [SMILES: *C1CC(F)(F)CO1 ; *N1CCOCC1]

336. The compound of any one of claims 299-327, wherein X¹ is —(X²)ₘ-L¹-R⁵, wherein:
m is 0 or 1;
X² is —N(R^{N})— or —O—;
U is a bond or C₁₋₆ alkylene optionally substituted with from 1-3 R^{a}; and
R⁵ is —R^{g2}-R^{Y}.

337. The compound of claim 336, wherein the —R^{g2} group present in R⁵ is 1,3-phenylene or 1,4-phenylene, each optionally substituted with from 1-4 R^{c}, such as wherein —R^{g2} is 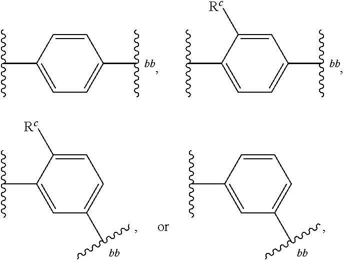
wherein bb is the point of attachment to R^{Y}.

338. The compound of claim 336 or 337, wherein the R^{Y} group present in R⁵ is —R^{g}.

339. The compound of any one of claims 336-338, wherein the R^{Y} group present in R⁵ is heterocyclyl including from 4-8, such as 4-6, ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}, such as wherein R^{Y} is

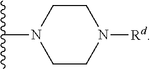

340. The compound of any one of claims 329-337, wherein X¹ is —X²-L¹-R⁵, wherein:
X² is —N(R^{N})—, —O—, —N(R^{N})C(═O)—*, —N(R^{N})S(O)₂—, —N(R^{N})C(═O)O—*, or —N(R^{N})C(═O)N(R^{N})—*;
L¹ is C₁₋₆ alkylene optionally substituted with from 1-3 R^{a}; and
R⁵ is H, halo, C₁₋₆ alkoxy optionally substituted with from 1-3 R^{a}, or —OH.

341. The compound of claim 340, wherein R⁵ is H.

342. The compound of claim 340, wherein R⁵ is halo, such as —F.

343. The compound of claim 340, wherein R⁵ is C₁₋₆ alkoxy optionally substituted with from 1-3 R^{a}, such as wherein R⁵ is C₁₋₃ alkoxy such as methoxy.

344. The compound of claim 340, wherein R⁵ is —OH.

345. The compound of any one of claim 329 or 331-339, wherein m is 0.

346. The compound of any one of claim 329 or 331-339, wherein m is 1.

347. The compound of any one of claim 329, 331-334 or 346 wherein X² is —N(R^{N})—, such as N(H).

348. The compound of any one of claim 329, 331-334 or 346, wherein X² is —O—.

349. The compound of any one of claim 329, 331-335 or 340-344, wherein X² is —N(R^{N})C(═O)—*, such as —N(H)C(═O)—*.

350. The compound of any one of claim 329, 331-335 or 340-344, wherein X² is —N(R^{N})S(O)₂—, such as —N(H)S(O)₂—*.

351. The compound of any one of claim 329, 331-335 or 340-344, wherein X² is —N(R^{N})C(═O)O—*, or —N(R^{N})C(═O)N(R^{N})*, such as —N(H)C(═O)O—* or —N(H)C(═O)N(H)—*.

352. The compound of any one of claims 330-335, wherein X² is

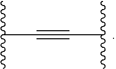 [SMILES: *C#C*]

353. The compound of any one of claims 330-335, wherein X² is

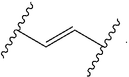 [SMILES: */C=C/*]

354. The compound of any one of claim 263-274 or 280-288, wherein L¹ is a bond.

355. The compound of any one of claims 328-353, wherein L¹ is C₁₋₃ alkylene, such as —CH₂—, —CH₂CH₂—, or —CH(Me)-.

356. The compound of any one of claims 328-353, wherein L¹ is branched C₃₋₆ alkylene, such as 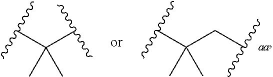
wherein aa is the point of attachment to R⁵.

357. The compound of any one of claims 299-327, wherein X¹ is -L¹-R⁵, wherein L¹ is C₁₋₆ alkylene optionally substituted with from 1-3 R^{a}; and R⁵ is -L⁵-R^{g}.

358. The compound of claim 357, wherein R⁵ is —O—R^{g}.

359. The compound of claim 357 or 358, wherein R⁵ is —O-(phenyl), wherein the phenyl is optionally substituted with from 1-2 R^{c}.

360. The compound of any one of claims 357-359, wherein L¹ is C₁₋₃ alkylene, such as —CH₂—, —CH₂CH₂—, or —CH(Me)-.

361. The compound of any one of claims 273-360, wherein R^{1c} is H.

362. The compound of any one of claims 273-361, wherein R^{2a} and R^{2b} are both H.

363. The compound of any one of claims 273-361, wherein R^{2a} is a substituent that is other than H.

364. The compound of any one of claim 273-361 or 363, wherein R^{2a} is C₁₋₆ alkyl which is optionally substituted with from 1-6 R^{a}, such as wherein R^{2a} is C₁₋₃ alkyl, such as methyl or ethyl.

365. The compound of claim 363 or 364, wherein R^{2b} is H.

366. The compound of any one of claims 273-365, wherein R^{3a} and R^{3b} are both H.

367. The compound of any one of claims 273-365, wherein R^{3a} is a substituent that is other than H.

368. The compound of any one of claim 273-365 or 367, wherein R^{3a} is C₁₋₆ alkyl which is optionally substituted with from 1-6 R^{a}, such as wherein R^{3a} is C₁₋₃ alkyl, such as methyl or ethyl.

369. The compound of any one of claim 273-365 or 367, wherein R^{3a} is C₁₋₃ alkyl substituted with from 1-3 independently selected halo, such as wherein R^{3a} is —CH₂F, —CHF₂, —CF₃, —CH₂CHF₂, or —CH₂CH₂F.

370. The compound of any one of claim 273-365 or 367, wherein R^{3a} is C₁₋₃ alkyl substituted with C₁₋₄ alkoxy, C₁₋₄ haloalkoxy, or NR^{e}R^{f}, such as wherein R^{3a} is —CH₂OMe, —CH₂CH₂OMe, —CH(Me)CH₂OMe, —CH₂CH(Me)OMe, -CH₂OEt, —CH₂NR^{e}R^{f} (e.g., —CH₂N(CF₃)Me), or —CH₂CH₂NR^{e}R^{f} (e.g., —CH₂CH₂NMe₂).

371. The compound of any one of claim 273-365 or 367, wherein R^{a} is selected from the group consisting of:
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
C₃₋₆ cycloalkyl optionally substituted with from 1-4 R^{c}.

372. The compound of any one of claim 273-365 or 367, wherein R^{3a} is —(C₁₋₃ alkylene)-R^{g} or —(C₁₋₃ alkylene)-O—R^{g}, and optionally the R^{g} group of R^{3a} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}.

373. The compound of claim 273-365, 367 or 372, wherein R^{3a} is —CH₂—R^{g}, or —CH₂CH₂R^{g}, wherein R^{g} is 1,4-dioxanyl.

374. The compound of any one of claim 273-365 or 367, wherein R^{3a} is-(L^{g})_{g}-R^{W}.

375. The compound of any one of claim 273-365, 367 or 374, wherein R^{3a} is-CH₂CH₂—R^{W}, wherein the R^{W} group is C(═O)—CH═CH₂, or —NHC(═O)—CH═CH₂.

376. The compound of any one of claim 273-365, 367 or 374-375, wherein R^{3a} is 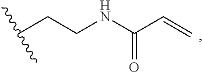 [SMILES: *CCNC(=O)C=C]
such as

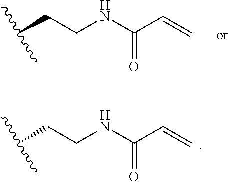

377. The compound of any one of claim 273-365, or 367, wherein R^{3a} is -(L^{g})_{g}-R^{g2}-R^{W}.

378. The compound of any one of claim 273-365, 367 or 377, wherein R^{3a} is —CH₂—R^{g2}-R^{W}, wherein the R^{g2} group is 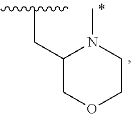 [SMILES: *CC1COCCN1C]
such as 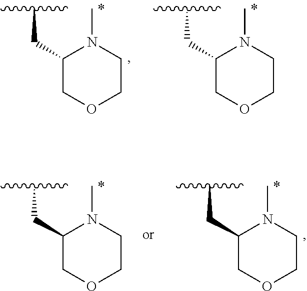
wherein the waveline represents the point of attachment to —CH₂— and the asterisk represents the point of attachment to R^{W}; and optionally the R^{W} group is C(═O)—CH═CH₂.

379. The compound of any one of claim 273-365, 367 or 377-378, wherein R^{3a} is 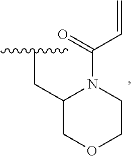 [SMILES: *CC1COCCN1C(=O)C=C]
such as

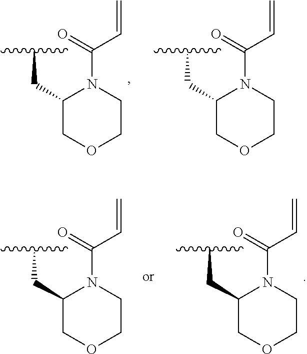

380. The compound of claim 273-365, or 367-379, wherein R^{3b} is H.

381. The compound of any one of claim 273-365, or 367-379, wherein R^{3b} is C₁₋₃ alkyl, such as methyl.

382. The compound of any one of claims 273-365, wherein R^{3a} and R^{3b}, together with the Ring B ring atom to which each is attached, form a fused saturated ring of 4-8 ring atoms;
wherein from 0-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-8 ring atoms is optionally substituted with from 1-4 substituents independently selected from the group consisting of oxo, R^{c}, and R^{W}.

383. The compound of any one of claim 273-365 or 382, wherein R^{3a} and R^{3b}, together with the Ring B ring atom to which each is attached, form: 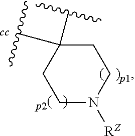
which is optionally substituted with from 1-2 substituents independently selected from the group consisting of oxo and R^{C}, wherein:
p1 and p2 are independently 0, 1, or 2;
R^{Z} is H, R^{d}, C(═O)—W, or S(O)₂W; and
cc represents the point of attachment to C(R^{2a}R^{2b}).

384. The compound of any one of claim 273-365 or 382-383, wherein R^{3a} and R^{3b}, together with the Ring B ring atom to which each is attached, form 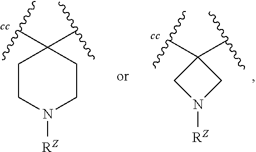
wherein R^{Z} is H, R^{d}, C(═O)—W, or S(O)₂W; and cc represents the point of attachment to C(R^{2a}R^{2b}).

385. The compound of any one of claim 273-365 or 382-383, wherein R^{3a} and R^{3b}, together with the Ring B ring atom to which each is attached, form a fused ring selected from the group consisting of: 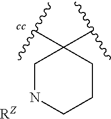
such as 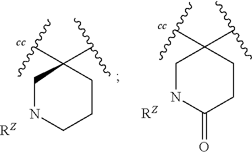
such as 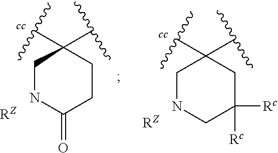
such as 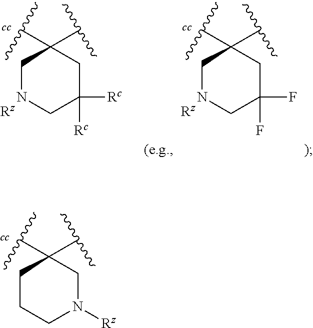
such as 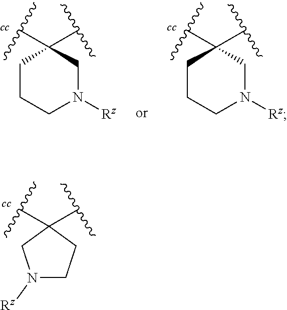
such as 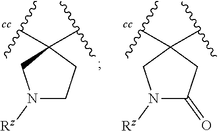
such as 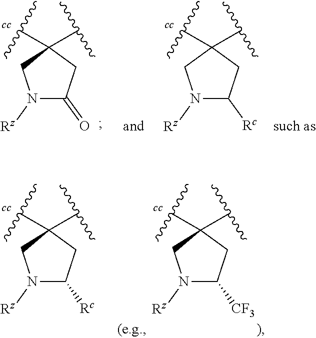
wherein R^{Z} is H, R^{d}, C(═O)—W, or S(O)₂W; and cc represents the point of attachment to C(R^{2a}R^{2b}).

386. The compound any one of claims 383-385, wherein R^{Z} is H.

387. The compound of any one of claims 383-385, wherein R^{Z} is C₁₋₆ alkyl optionally substituted with from 1-3 independently selected R^{a}.

388. The compound of any one of claims 383-385, wherein R^{Z} is C(═O)—W or S(O)₂W, optionally wherein W is C2-4 alkenyl.

389. The compound of any one of claims 273-365, wherein R^{3a} and R^{3b} together with the Ring B ring atom to which each is attached form a fused C3, cycloalkyl, wherein the fused C₃₋₆ cycloalkyl is optionally substituted with from 1-2 R^{c}.

390. The compound of any one of claims 273-365, wherein R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

391. The compound of any one of claims 273-361, wherein R^{2a} and R^{3a} taken together with the Ring B ring atoms to which each is attached, form a fused C₃₋₆ (e.g., C₃ or C₄) cycloalkyl which is optionally substituted with from 1-2 R^{c}.

392. The compound of any one of claims 273-362, wherein R^{2b} and R^{3b} are each H.

393. The compound of any one of claims 273-362, wherein R^{1c}, R^{2a}, and R^{2b} are each H; R^{3a} is C₁₋₃ alkyl optionally substituted with from 1-3 R^{a}; and R^{3b} is H, optionally each R^{a} substituent present in R^{3a} is independently selected from the group consisting of: halo, C₁₋₄ alkoxy, and C₁₋₄ haloalkoxy.

394. The compound of any one of claim 273-362 or 393, wherein R^{1c}, R^{2a}, and R^{2b} are each H; and R^{3a} and R^{3b} are independently selected C₁₋₃ alkyl.

395. The compound of any one of claims 273-362, wherein R^{1c}, R^{2a}, and R^{2b} are each H: R^{3a}, is —R^{g}, —(C₁₋₃ alkylene)-R^{g}, or —(C₁₋₃ alkylene)-O—R^{g},
optionally wherein the R^{g} group of R^{3a} is:
C₃₋₆ cycloalkyl optionally substituted with from 1-4 R^{c}, or
heterocyclyl including from 4-6 ring atoms, wherein from 1-3 ring atoms are heteroatoms, each independently selected from the group consisting of N, N(H), N(R^{d}), O, and S(O)₀₋₂, and wherein the heterocyclyl is optionally substituted with from 1-4 substituents independently selected from the group consisting of oxo and R^{c}; and
R^{3b} is H.

396. The compound of any one of claims 273-362, wherein R^{1c}, R^{2a}, and R^{2b} are each H; and R^{3a} and R^{3b} taken together with the Ring B ring carbon atom to which each is attached form a fused C₃₋₆ (such as C₃ or C₄) cycloalkyl, wherein the fused cycloalkyl ring is optionally substituted with from 1-2 R^{c}.

397. The compound of any one of claims 273-362, wherein R^{1c}, R^{2a}, and R^{2b} are each H; and R^{3a} and R^{3b} together with the Ring B ring atom to which each is attached, form a fused saturated ring of 4-6 ring atoms;
wherein from 1-2 of the ring atoms are each an independently selected heteroatom, wherein each of the independently selected heteroatoms is selected from the group consisting of N, NH, N(R^{d}), O, and S(O)₀₋₂; and
wherein the fused saturated ring of 4-6 ring atoms is optionally substituted with from 1-2 substituents independently selected from the group consisting of oxo and R^{c}.

398. The compound of any one of claims 273-361, wherein R^{1c} is H, R^{2a} and R^{3a} taken together with the Ring B ring atoms to which each is attached, form a fused C₃₋₆ (e.g., C₃ or C₄) cycloalkyl which is optionally substituted with from 1-2 R^{c}; and R^{2b} and R^{3b} are each H.

399. The compound of any one of claims 273-361, wherein R^{1c} is H; R^{2a} and R^{3b} combine to form a double bond between the Ring B atoms to which each is attached; and R^{2b} is H; and R^{3b} is -(L^{g})_{g}-R^{g}.

400. The compound of any one of claim 273-361 or 399, wherein R^{1c} is H; R^{2a} and R^{3a} combine to form a double bond between the Ring B atoms to which each is attached; and R^{2b} is H; and R^{3b} is 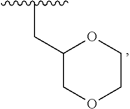 [SMILES: *CC1COCCO1]
such as

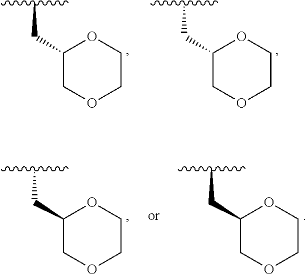

401. The compound of any one of claims 273-362, wherein R^{1c}, R^{2a}, R^{2b}, R^{3a}, and R^{3b} are each H.

402. The compound of any one of claims 273-401, wherein R⁴ is H.

403. The compound of any one of claims 273-402, wherein Ring A is 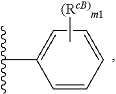
wherein each R^{cB} is an independently selected R^{c}; and m1 is 0, 1, 2, 3, or 4.

404. The compound of claim 403, wherein m1 is 1, 2, or 3, such as 1 or 2.

405. The compound of any one of claims 273-404, wherein Ring A is 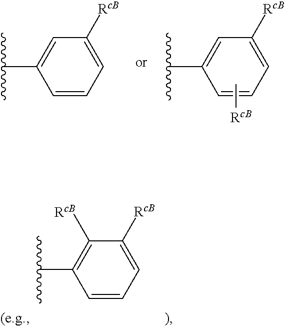
wherein each R^{cB} is an independently selected R^{c}.

406. The compound of any one of claims 273-405, wherein Ring A is

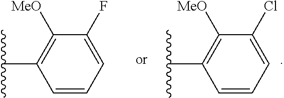 [SMILES: *c1cccc(F)c1OC ; *c1cccc(Cl)c1OC]

407. The compound of an one of claims 273-404, wherein Ring A is selected from the group consisting of: 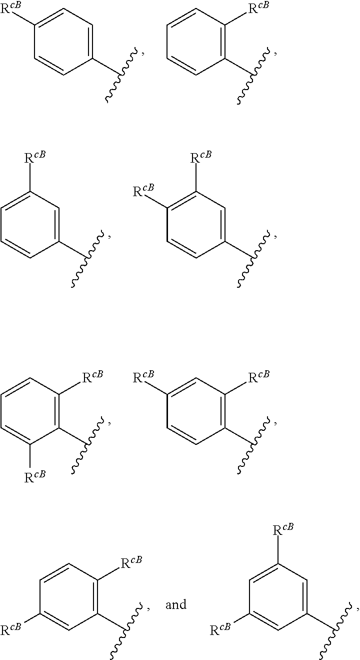
wherein each R^{cB} is an independently selected R^{c}.

408. The compound of any one of claims 403-407, wherein each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

409. The compound of any one of claim 273402, wherein Ring A is bicyclic heteroaryl including from 9-10 ring atoms, wherein from 14 ring atoms are heteroatoms, each independently selected from the group consisting of N, N(H), N(R^{d}), O, and S(O)₀₋₂, and wherein the heteroaryl is optionally substituted with from 1-4 R^{c}, such as wherein: Ring A is selected from the group consisting of: 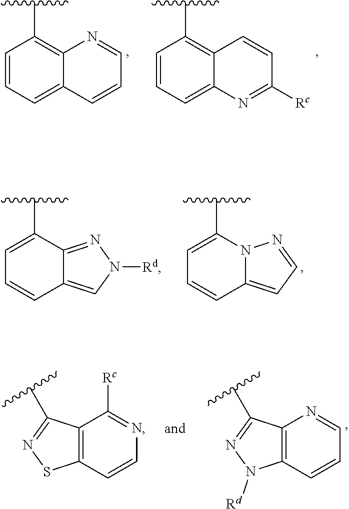
each of which is further optionally substituted with R^{c}.

410. The compound of any one of claim 1409, wherein the 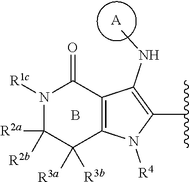
moiety is

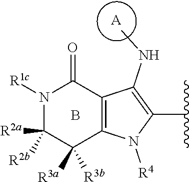

411. The compound of any one of claims 1-409, wherein the 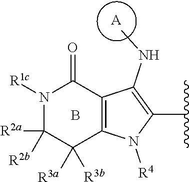
moiety is

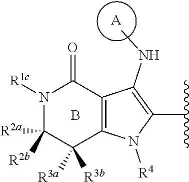

412. The compound of claim 1, wherein the compound is selected from the group consisting of the compounds delineated in Table C1, or a pharmaceutically acceptable salt thereof.

413. A pharmaceutical composition comprising a compound of any one of claims 1-412, or a pharmaceutically acceptable salt thereof, and pharmaceutically acceptable diluent or carrier.

414. A method for treating cancer in a subject in need thereof, the method comprising administering to the subject a therapeutically effective amount of a compound of any one of claims 1-412, or a pharmaceutically acceptable salt thereof, or a pharmaceutical composition according to claim 413.

415. A method for treating cancer in a subject in need thereof, the method comprising (a) determining that the cancer is associated with a dysregulation of an EGFR gene, an EGFR kinase, or expression or activity or level of any of the same; and (b) administering to the subject a therapeutically effective amount of a compound of any one of claims 1-412, or a pharmaceutically acceptable salt thereof, or a pharmaceutical composition according to claim 413.

416. A method of treating an EGFR-associated cancer in a subject, the method comprising administering to a subject identified or diagnosed as having an EGFR-associated cancer a therapeutically effective amount of a compound of any one of claims 1-412 or a pharmaceutically acceptable salt thereof, or a pharmaceutical composition according to claim 383.

417. A method of treating an EGFR-associated cancer in a subject, the method comprising:
(a) determining that the cancer in the subject is an EGFR-associated cancer; and
(b) administering to the subject a therapeutically effective amount of a compound of any one of claims 1-412 or a pharmaceutically acceptable salt thereof, or a pharmaceutical composition according to claim 413.

418. A method of treating a subject, the method comprising administering a therapeutically effective amount of a compound of any one of claims 1-412 or a pharmaceutically acceptable salt thereof, or a pharmaceutical composition according to claim 413, to a subject having a clinical record that indicates that the subject has a dysregulation of an EGFR gene, an EGFR kinase, or expression or activity or level of any of the same.

419. The method of any one of claims 415 and 417, wherein the step of determining that the cancer in the subject is an EGFR-associated cancer includes performing an assay to detect dysregulation in an EGFR gene, an EGFR kinase protein, or expression or activity or level of any of the same in a sample from the subject.

420. The method of claim 419, further comprising obtaining a sample from the subject.

421. The method of claim 420, wherein the sample is a biopsy sample.

422. The method of any one of claims 419-421, wherein the assay is selected from the group consisting of sequencing, immunohistochemistry, enzyme-linked immunosorbent assay, and fluorescence in situ hybridization (FISH).

423. The method of claim 422, wherein the FISH is break apart FISH analysis.

424. The method of claim 422, wherein the sequencing is pyrosequencing or next generation sequencing.

425. The method of any one of claims 415, 418, and 419, wherein the dysregulation in an EGFR gene, an EGFR kinase protein, or expression or activity or level of any of the same is one or more point mutations in the EGFR gene.

426. The method of claim 425, wherein the one or more point mutations in an EGFR gene results in the translation of an EGFR protein having one or more amino acid substitutions at one or more of the following amino acid positions exemplified in Table 1a and 1b.

427. The method of claim 426, wherein the one or more point mutations is selected from the mutations in Table 1a and 1b (e.g., L858R, G719S, G719C, G719A, L861Q, a deletion in exon 19 and/or an insertion in exon 20).

428. The method of claim 426, wherein the one or more point mutations is an EGFR inhibitor resistance mutation (e.g., L718Q, L747S, D761Y, T790M, C797S, T854A).

429. The method of claim 426, wherein the one or more point mutations in an EGFR gene include a deletion in exon 19 of a human EGFR gene.

430. The method of claim 426, wherein the one or more mutations is an EGFR insertion in exon 20 of a human EGFR gene.

431. The method of claim 428, wherein the insertion in exon 20 of a human EGFR gene is selected from: V769_D770insX, D770_N771insX, N771_P772insX, P772_H773insX, and H773_V774insX.

432. The method of claim 428 or 429, wherein the insertion in exon 20 of a human EGFR gene is selected from: Y772_A775dup, A775_G776insYVMA, G776delinsVC, G776delinsVV, V777_G778insGSP, and P780_Y781insGSP.

433. The method of any one of claims 426, 427 and 329-432, wherein the EGFR-associated cancer is selected from the group consisting of: oral cancer, oropharyngeal cancer, nasopharyngeal cancer, respiratory cancer, urogenital cancer, gastrointestinal cancer, central or peripheral nervous system tissue cancer, an endocrine or neuroendocrine cancer, a hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma Li-Fraumeni tumor, thyroid cancer, parathyroid cancer, pituitary tumors, adrenal gland tumors, osteogenic sarcoma tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer and skin cancer.

434. The method of any one of claims 417 and 419-433, wherein the EGFR-associated cancer is selected from the group consisting of: lung cancer, pancreatic cancer, head and neck cancer, melanoma, colon cancer, renal cancer, leukemia, glioblastoma, or breast cancer.

435. The method of claim 433 or 434, wherein the lung cancer is non-small cell lung cancer.

436. The method of any one of claims 414-435, wherein the cancer is a HER2-associated cancer.

437. The method of claim 436, wherein the HER2-associated cancer is associated with a dysregulation of a HER2 gene, a HER2 kinase, or expression or activity or level of any of the same.

438. The method of any one of claims 436 and 437, wherein determining that the cancer in the subject is a HER2-associated cancer includes performing an assay to detect dysregulation in a HER2 gene, a HER2 kinase protein, or expression or activity or level of any of the same in a sample from the subject.

439. The method of claim 438, further comprising obtaining a sample from the subject.

440. The method of claim 439, wherein the sample is a biopsy sample.

441. The method of any one of claims 438-440, wherein the assay is selected from the group consisting of sequencing, immunohistochemistry, enzyme-linked immunosorbent assay, and fluorescence in situ hybridization (FISH).

442. The method of claim 441, wherein the sequencing is pyrosequencing or next generation sequencing.

443. The method of any one of claims 437-442, wherein the dysregulation in a HER12 gene, a HER2 kinase protein, or expression or activity or level of any of the same is one or more point mutations in the HER2 gene.

444. The method of claim 443, wherein the one or more point mutations in a HER2 gene results in the translation of a HER2 protein having one or more amino acid substitutions at one or more of the following amino acid positions exemplified in Table 3.

445. The method of claim 444, wherein the one or more point mutations is selected from the mutations in Table 3 (e.g., S310F, S310Y, R678Q, R678W, R678P, I767M, V773M, V777L, and V842I).

446. The method of any one of claims 414-445, wherein the cancer is selected from the group consisting of: non-small cell lung cancer, pancreatic cancer, and colorectal cancer.

447. The method of any one of claims 414-446, further comprising administering an additional therapy or therapeutic agent to the subject.

448. The method of claim 447, wherein the additional therapy or therapeutic agent is selected from radiotherapy, cytotoxic chemotherapeutics, kinase targeted-therapeutics, apoptosis modulators, signal transduction inhibitors, immune-targeted therapies, and angiogenesis-targeted therapies.

449. The method of claim 448, wherein said additional therapeutic agent is selected from one or more kinase targeted therapeutics.

450. The method of claim 449, wherein said additional therapeutic agent is a tyrosine kinase inhibitor.

451. The method of claim 450, wherein said additional therapeutic agent is a second EGFR inhibitor.

452. The method of claim 447, wherein said additional therapeutic agent is selected from osimertinib, gefitinib, erlotinib, afatinib, lapatinib, neratinib, AZD-9291, CL-387785, CO-1686, WZ4002, and combinations thereof.

453. The method of claim 447, wherein said additional therapeutic agent is a second compound of any one of claims 1-412 or a pharmaceutically acceptable salt thereof, or a pharmaceutical composition according to claim 413.

454. The method of claim 447, wherein said additional therapeutic agent is a HER2 inhibitor.

455. The method of claim 454, wherein the HER2 inhibitor is selected from trastuzumab, pertuzumab, trastuzumab emtansine, lapatinib, KU004, neratinib, dacomitinib, afatinib, tucatinib, erlotinib, pyrotinib, poziotinib, CP-724714, CUDC-101, sapitinib (AZD8931), tanespimycin (17-AAG), IPI-504, PF299, pelitinib, S-22261 1, and AEE-788.

456. The method of any one of claims 447-455, wherein the compound of any one of claims 1-412 or a pharmaceutically acceptable salt thereof, or a pharmaceutical composition according to claim 413, and the additional therapeutic agent are administered simultaneously as separate dosages.

457. The method of any one of claims 447-455, wherein the compound of any one of claims 1-412 or a pharmaceutically acceptable salt thereof, or a pharmaceutical composition according to claim 413, and the additional therapeutic agent are administered as separate dosages sequentially in any order.

458. A method of treating a subject having a cancer, wherein the method comprises:
(a) administering one or more doses of a first EGFR inhibitor to the subject for a period of time;
(b) after (a), determining whether a cancer cell in a sample obtained from the subject has at least one EGFR inhibitor resistance mutation that confers increased resistance to a cancer cell or tumor to treatment with the first EGFR inhibitor of step (a); and
(c) administering a compound of any one of claims 1-412 or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first EGFR inhibitor of step (a); or
(d) administering additional doses of the first EGFR inhibitor of step (a) to the subject if the subject has not been determined to have a cancer cell that has at least one EGFR inhibitor resistance mutation that confers increased resistance to a cancer cell or tumor to treatment with the first EGFR inhibitor of step (a).

459. The method of claim 458, wherein the anticancer agent in step (c) is a second EGFR inhibitor, an immunotherapy, a HER2 inhibitor, or a combination thereof.

460. The method of claim 458, wherein the anticancer agent in step (c) is the first EGFR inhibitor administered in step (a).

461. The method of claim 458, wherein the subject is administered additional doses of the first inhibitor of EGFR of step (a), and the method further comprises (e) administering another anticancer agent to the subject.

462. The method of claim 461, wherein the anticancer agent of step (e) is a second EGFR inhibitor, an immunotherapy, or a combination thereof.

463. The method of claim 461, wherein the anticancer agent of step (e) is a compound of any one of claims 1-412 or a pharmaceutically acceptable salt thereof.

464. The method of any one of claims 458-463, wherein the EGFR inhibitor resistance mutation is a substitution at amino acid position 718, 747, 761, 790, 797, or 854 (e.g., L718Q, L747S, D761Y, T790M, C797S, T854A).

465. A method of treating an EGFR-associated cancer in a subject, the method comprising administering to a subject identified or diagnosed as having an EGFR-associated cancer that has one or more EGFR inhibitor resistance mutations a therapeutically effective amount of a compound of any one of claims 1-412 or a pharmaceutically acceptable salt thereof, or a pharmaceutical composition according to claim 413.

466. A method of treating an EGFR-associated cancer in a subject, the method comprising:
(a) determining that the cancer in the subject has one or more EGFR inhibitor resistance mutations; and
(b) administering to the subject a therapeutically effective amount of a compound of any one of claims 1-412 or a pharmaceutically acceptable salt thereof, or a pharmaceutical composition according to claim 413.

467. A method of treating a subject having a cancer, wherein the method comprises:
(a) determining whether a cancer cell in a sample obtained from a subject having a cancer and previously administered one or more doses of a first EGFR inhibitor has one or more EGFR inhibitor resistance mutations that confer increased resistance to a cancer cell or tumor to treatment with the first EGFR inhibitor that was previously administered to the subject; and
(b) administering a compound of any one of claims 1-412 or a pharmaceutically acceptable salt thereof, as a monotherapy or in conjunction with another anticancer agent to the subject if the subject has been determined to have a cancer cell that has at least one EGFR inhibitor resistance mutation that confers increased resistance to a cancer cell or tumor to treatment with the first modulator of EGFR that was previously administered to the subject; or
(c) administering additional doses of the first modulator of EGFR to the subject if the subject has not been determined to have a cancer cell that has at least one EGFR modulator resistance mutation that confers increased resistance to a cancer cell or tumor to treatment with the first modulator of EGFR previously administered to the subject.

468. The method of claim 467, wherein the anticancer agent of step (b) is a second EGFR inhibitor, an immunotherapy, a HER2 inhibitor, or a combination thereof.

469. The method of claim 467, wherein the anticancer agent of step (b) is the first EGFR inhibitor previously administered to the subject.

470. The method of claim 467, wherein the subject is administered additional doses of the first EGFR inhibitor previously administered to the subject, and the method further comprises (d) administering another anticancer agent to the subject.

471. The method of claim 470, wherein the anticancer agent of step (d) is a second EGFR inhibitor, an immunotherapy, or a combination thereof.

472. The method of claim 470, wherein the anticancer agent of step (d) is a compound of any one of claims 1-412 or a pharmaceutically acceptable salt thereof.

473. The method of claim 472, wherein the second EGFR inhibitor is selected from osimertinib, gefitinib, erlotinib, afatinib, lapatinib, neratinib, AZD-9291, CL-387785, CO-1686, WZ4002, and combinations thereof.

474. The method of any one of claims 465-473, wherein the cancer is selected from the group consisting of: non-small cell lung cancer, pancreatic cancer, and colorectal cancer.

475. The method of any one of claims 465-474, wherein the cancer is associated with a dysregulation of a HER2 gene, a HER2 kinase, or expression or activity or level of any of the same.

476. The method of claim 475, wherein the dysregulation in a HER2 gene, a HER2 kinase protein, or expression or activity or level of any of the same is one or more point mutations in the HER2 gene.

477. The method of claim 476, wherein the one or more point mutations in a HER2 gene results in the translation of a HER2 protein having one or more amino acid substitutions at one or more of the following amino acid positions exemplified in Table 3.

478. The method of claim 477, wherein the one or more point mutations is selected from the mutations in Table 3 (e.g., S310F, S310Y, R678Q, R678W, R678P, I767M, V773M, V777L, and V842I).

479. A method for modulating EGFR in a mammalian cell, the method comprising contacting the mammalian cell with an effective amount of a compound of any one of claims 1-412, or a pharmaceutically acceptable salt thereof.

480. The method of claim 479, wherein the contacting occurs in vivo.

481. The method of claim 479, wherein the contacting occurs in vitro.

482. The method of any one of claims 479-481, wherein the mammalian cell is a mammalian cancer cell.

483. The method of claim 482, wherein the mammalian cancer cell is a mammalian EGFR-associated cancer cell.

484. The method of any one of claims 479-483, wherein the cell has a dysregulation of an EGFR gene, an EGFR kinase protein, or expression or activity or level of any of the same.

485. The method of claim 484, wherein the dysregulation in an EGFR gene, an EGFR kinase protein, or expression or activity or level of any of the same is one or more point mutations in the EGFR gene.

486. The method of claim 485, wherein the one or more point mutations in an EGFR gene results in the translation of an EGFR protein having one or more amino acid substitutions at one or more of the following amino acid positions exemplified in Table 1a and 1b.

487. The method of claim 486, wherein the one or more point mutations is selected from the mutations in Table 1a and 1b (e.g., L858R, G719S, G719C, G719A, L861Q, a deletion in exon 19 and/or an insertion in exon 20).

488. The method of claim 485, wherein the one or more point mutations is an EGFR inhibitor resistance mutation (e.g., L718Q, L747S, D761Y, T790M, C797S, T854A).

489. The method of claim 485, wherein the one or more point mutations in an EGFR gene include a deletion in exon 19 of a human EGFR gene.

490. The method of claim 485, wherein the one or more point mutations is an EGFR insertion in exon 20 of a human EGFR gene.

491. The method of claim 490, wherein the insertion in exon 20 of a human EGFR gene is selected from: A767_V769insX, V769_D770insX, D770_N771insX, N771_P772insX, P772_H773insX, and H773_V774insX.

492. The method of claim 491, wherein the insertion in exon 20 of a human EGFR gene is selected from: A767_V769dupASV, V769_D770insASV, D770_N771insNPG, D770_N771insNPY, D770_N771insSVD, D770_N771insGL, N771_H773dupNPH, N771_P772insN, N771_P772insH, N771_P772insV, P772_H773insDNP, P772_H773insPNP, H773_V774insNPH, H773_V774insH, H773_V774insPH, H773_V774insAH, and P772_H773insPNP.

493. A method for treating cancer in a subject in need thereof, the method comprising (a) determining that the cancer is associated with a dysregulation of a HER2 gene, a HER2 kinase, or expression or activity or level of any of the same; and (b) administering to the subject a therapeutically effective amount of a compound of any one of claims 1-412, or a pharmaceutically acceptable salt thereof, or a pharmaceutical composition according to claim 413.

494. A method of treating a HER2-associated cancer in a subject, the method comprising administering to a subject identified or diagnosed as having a HER2-associated cancer a therapeutically effective amount of a compound of any one of claims 1-412 or a pharmaceutically acceptable salt thereof, or a pharmaceutical composition according to claim 413.

495. A method of treating a HER2-associated cancer in a subject, the method comprising:
(a) determining that the cancer in the subject is a HER2-associated cancer; and
(b) administering to the subject a therapeutically effective amount of a compound of any one of claims 1-412 or a pharmaceutically acceptable salt thereof, or a pharmaceutical composition according to claim 413.

496. A method of treating a subject, the method comprising administering a therapeutically effective amount of a compound of any one of claims 1-412 or a pharmaceutically acceptable salt thereof, or a pharmaceutical composition according to claim 413, to a subject having a clinical record that indicates that the subject has a dysregulation of a HER2 gene, a HER2 kinase, or expression or activity or level of any of the same.

497. The method of any one of claims 493 and 495, wherein the step of determining that the cancer in the subject is a HER2-associated cancer includes performing an assay to detect dysregulation in a HER2 gene, a HER2 kinase protein, or expression or activity or level of any of the same in a sample from the subject.

498. The method of claim 497, further comprising obtaining a sample from the subject.

499. The method of claim 498, wherein the sample is a biopsy sample.

500. The method of any one of claims 493-499, wherein the assay is selected from the group consisting of sequencing, immunohistochemistry, enzyme-linked immunosorbent assay, and fluorescence in situ hybridization (FISH).

501. The method of claim 500, wherein the FISH is break apart FISH analysis.

502. The method of claim 500, wherein the sequencing is pyrosequencing or next generation sequencing.

503. The method of any one of claims 493, 496, and 497, wherein the dysregulation in a HER2 gene, a HER2 kinase protein, or expression or activity or level of any of the same is one or more point mutations in the HER2 gene.

504. The method of claim 503, wherein the one or more point mutations in a HER2 gene results in the translation of a HER2 protein having one or more amino acid substitutions at one or more of the following amino acid positions exemplified in Table 3.

505. The method of claim 503, wherein the one or more point mutations is selected from the mutations in Table 3 (e.g., S310F, S310Y, R678Q, R678W, R678P, 1767M, V773M, V777L, and V842I).

506. The method of any one of claims 492, 495, and 496, wherein the dysregulation in a HER2 gene, a HER2 kinase protein, or expression or activity or level of any of the same is an insertion in exon 20 of the human HER2 gene.

507. The method of claim 506, wherein the insertion in exon 20 of the human HER2 gene is deletions at an amino acid position selected from: 774, 775, 776, 777, 778, and 780.

508. The method of claim 507, wherein the insertion in exon 20 of a human HER2 gene is selected from: M774AYVM, M774del insWLV, A775_G776insYVMA, A775_G776insAVMA, A775_G776insSVMA, A775_G776insVAG, A775insV G776C, A775_G776insI, G776del insVC2, G776del insVV, G776del insLC, G776C V777insC, G776C V777insV, V777 G778insCG, G778 S779insCPG, and P780 Y781insGSP.

509. The method of any one of claims 494, 495, and 497, wherein the HER2-associated cancer is selected from the group consisting of: colon cancer, lung cancer, or breast cancer.

510. The method of claim 509, wherein the lung cancer is non-small cell lung cancer.

511. The method of any one of claims 496-510, further comprising administering an additional therapy or therapeutic agent to the subject.

512. The method of claim 511, wherein the additional therapy or therapeutic agent is selected from radiotherapy, cytotoxic chemotherapeutics, kinase targeted-therapeutics, apoptosis modulators, signal transduction inhibitors, immune-targeted therapies and angiogenesis-targeted therapies.

513. The method of claim 511, wherein said additional therapeutic agent is a second compound of any one of claims 1-412 or a pharmaceutically acceptable salt thereof, or a pharmaceutical composition according to claim 413.

514. The method of claim 511, wherein said additional therapeutic agent is selected from one or more kinase targeted therapeutics.

515. The method of claim 511, wherein said additional therapeutic agent is a tyrosine kinase inhibitor.

516. The method of claim 511, wherein said additional therapeutic agent is an EGFR inhibitor.

517. The method of claim 511, wherein said additional therapeutic agent is selected from osimertinib, gefitinib, erlotinib, afatinib, lapatinib, neratinib, AZD-9291, CL-387785, CO-1686, WZ4002, and combinations thereof.

518. The method of claim 511, wherein said additional therapeutic agent is a HER2 inhibitor.

519. The method of claim 518, wherein the HER2 inhibitor is selected from trastuzumab, pertuzumab, trastuzumab emtansine, lapatinib, KU004, neratinib, dacomitinib, afatinib, tucatinib, erlotinib, pyrotinib, poziotinib, CP-724714, CUDC-101, sapitinib (AZD8931), tanespimycin (17-AAG), IPI-504, PF299, pelitinib, S-22261 1, and AEE-788.

520. The method of any one of claims 514-519, wherein the compound of any one of claims 1-412 or a pharmaceutically acceptable salt thereof, or a pharmaceutical composition according to claim 413, and the additional therapeutic agent are administered simultaneously as separate dosages.

521. The method of any one of claims 514-519, wherein the compound of any one of claims 1-412 or a pharmaceutically acceptable salt thereof, or a pharmaceutical composition according to claim 413, and the additional therapeutic agent are administered as separate dosages sequentially in any order.
